(19) United States
(12) Patent Application Publication
(10) Pub. No.: US 2025/0179114 A1
(43) Pub. Date: Jun. 5, 2025

(54) Anti-Inflammatory Compound and Use Thereof

(71) Applicant: DUALITY BIOLOGICS (SUZHOU) CO., LTD., Jiangsu (CN)

(72) Inventors: Yu ZHANG, Shanghai (CN); Bing LI, Shanghai (CN); Xi LI, Shanghai (CN); Haiqing HUA, Shanghai (CN); Jian LI, Shanghai (CN); Zhongyuan ZHU, Shanghai (CN)

(73) Assignee: DUALITY BIOLOGICS (SUZHOU) CO., LTD., Jiangsu (CN)

(21) Appl. No.: 18/691,346

(22) PCT Filed: Sep. 13, 2022
(86) PCT No.: PCT/CN2022/118306

§ 371 Date: Mar. 12, 2024

(30) Foreign Application Priority Data
Sep. 14, 2021 (CN) 202111084100.3
Dec. 23, 2021 (CN) 202111592481.6
Aug. 31, 2022 (CN) 202211061074.7

(51) Int. Cl. C07J 71/00; A61K 47/68; A61P 19/02; C07K 5/083

(52) U.S. Cl. CPC C07J 71/0031; A61K 47/6803; A61P 19/02; C07K 5/0806

(57) ABSTRACT

Provided are an anti-inflammatory compound and use thereof. Specifically, provided are the compound or a tautomer, a mesomer, a racemate, an enantiomer and a diastereoisomer thereof, or a mixture form thereof, or a pharmaceutically acceptable salt thereof. Further provided are a method for preparing the compound and use thereof.

### TECHNICAL FIELD

The present application relates to the field of biomedicine, and in particular to an anti-inflammatory compound and use thereof:

### BACKGROUND

Inflammation is an adaptive response triggered by a variety of noxious stimuli and conditions, and it underlies many diseases related to the human immune system. Steroid compounds are a class of anti-inflammatory drugs that have the potential to influence immune system functions and treat or prevent diseases and/or conditions associated with glucocorticoid receptor signaling. However, some existing steroid compounds are not that potent in anti-inflammation, and others may have many undesirable side effects. Therefore, there is an urgent need to further develop antibody-drug conjugates and steroid compounds formed from various steroids as drugs that can exert better therapeutic effect or have better safety.

### SUMMARY

The present application provides a compound or a tautomer, a mesomer, a racemate, an enantiomer or a diastereoisomer thereof, or a mixture thereof, or a pharmaceutically acceptable salt thereof, which may have one or more effects selected from the group consisting of: (1) having the ability to influence the activity of immune cells; (2) the conjugate thereof having targeting effect; (3) having plasma stability; (4) having biological safety; (5) having the ability to influence cytokine release from immune cells; (6) having the ability to affect transcription of responsive genes of IFN signaling pathway; (7) having the ability to influence the degree of skin fibrosis; (8) having the ability to influence the number and/or proportion of dendritic cells; (9) having the ability to influence the collagen content of the skin; (10) having the ability to influence the GRE expression level; (11) having the ability to influence cytokine release from monocytes; (12) having the ability to influence contact hypersensitivity; (13) having the ability to influence skin swelling and (14) having the ability to influence arthritic symptoms.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II):

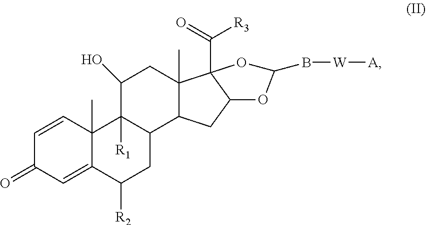

wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and each Y is independently selected from the group consisting of: hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂. In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1) or formula (II-a2):

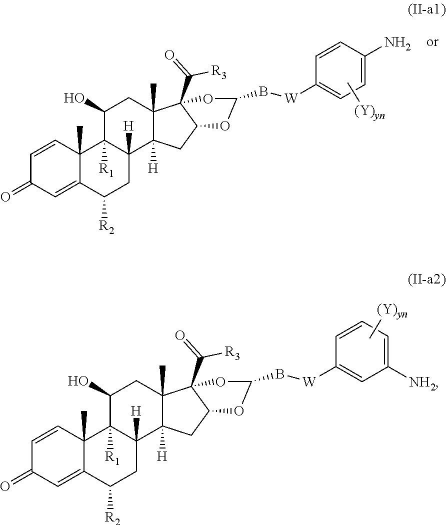

wherein
R₁, R₂, R₃ and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure selected from the present application.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

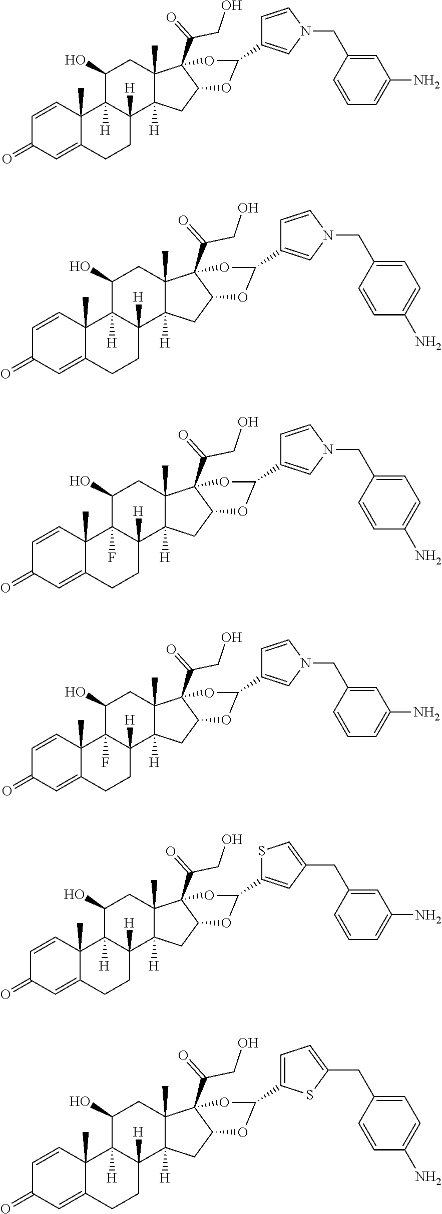

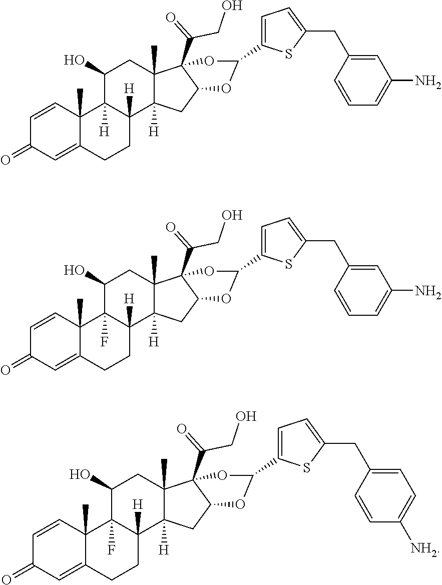

In one aspect, the present application provides a conjugate comprising the compound or the tautomer, the mesomer, the racemate, the enantiomer or the diastereoisomer thereof, or the mixture thereof, or the pharmaceutically acceptable salt thereof described herein.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-A):

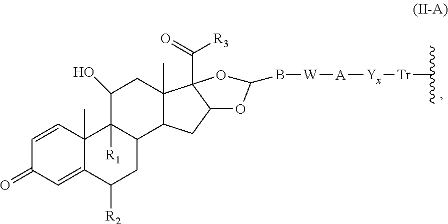

wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; Tr is absent or is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-A), formula (II-a2-A), formula (II-a3-A), or formula (II-a4-A):

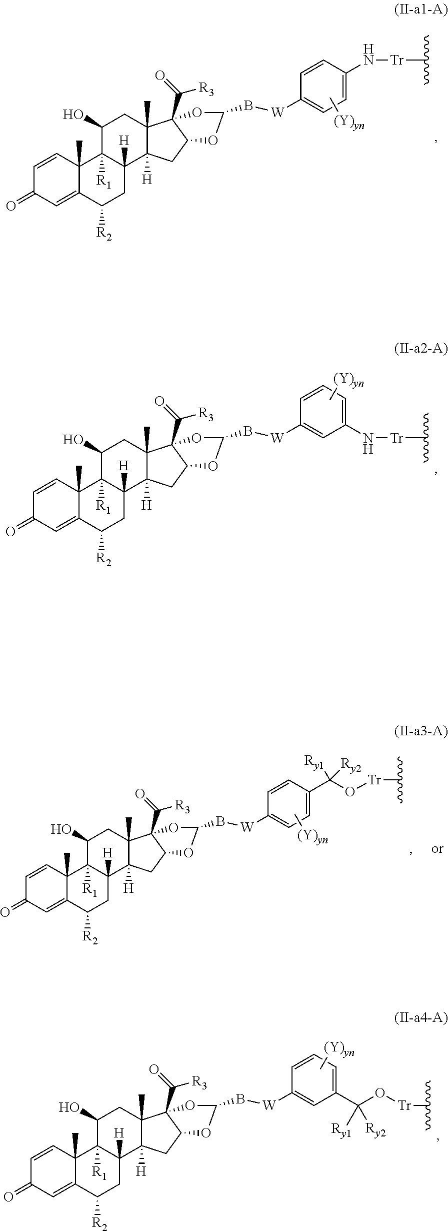

wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; Tr is absent or is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-B):

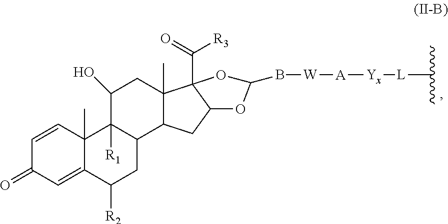

wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; L is absent or is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-B), formula (II-a2-B), formula (II-a3-B), or formula (II-a4-B):

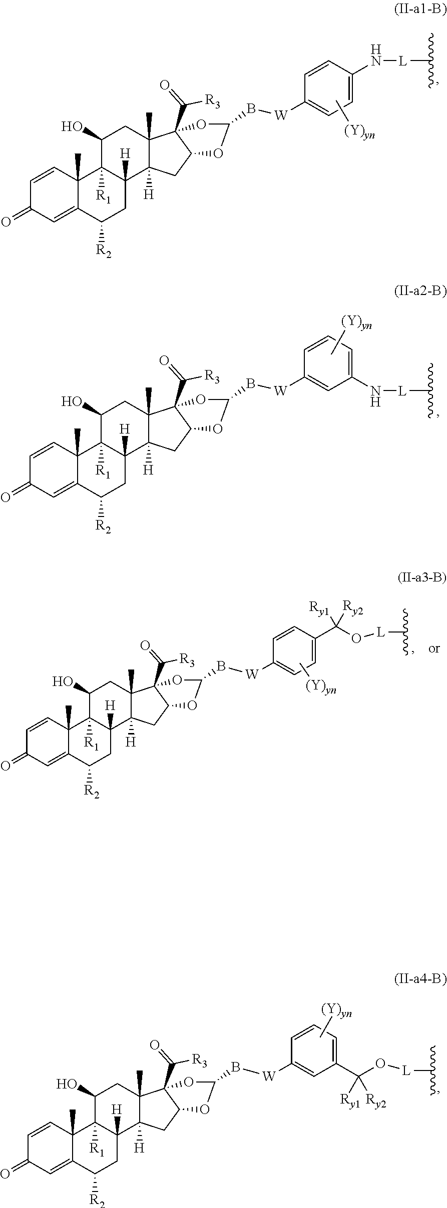

wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; L is absent or is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-C):

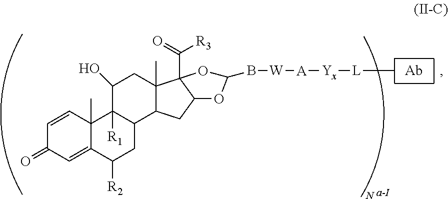

wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; L is absent or is any group; Ab is a ligand; N^{a-1} is a number equal to or greater than 0.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-C), formula (II-a2-C), formula (II-a3-C), or formula (II-a4-C):

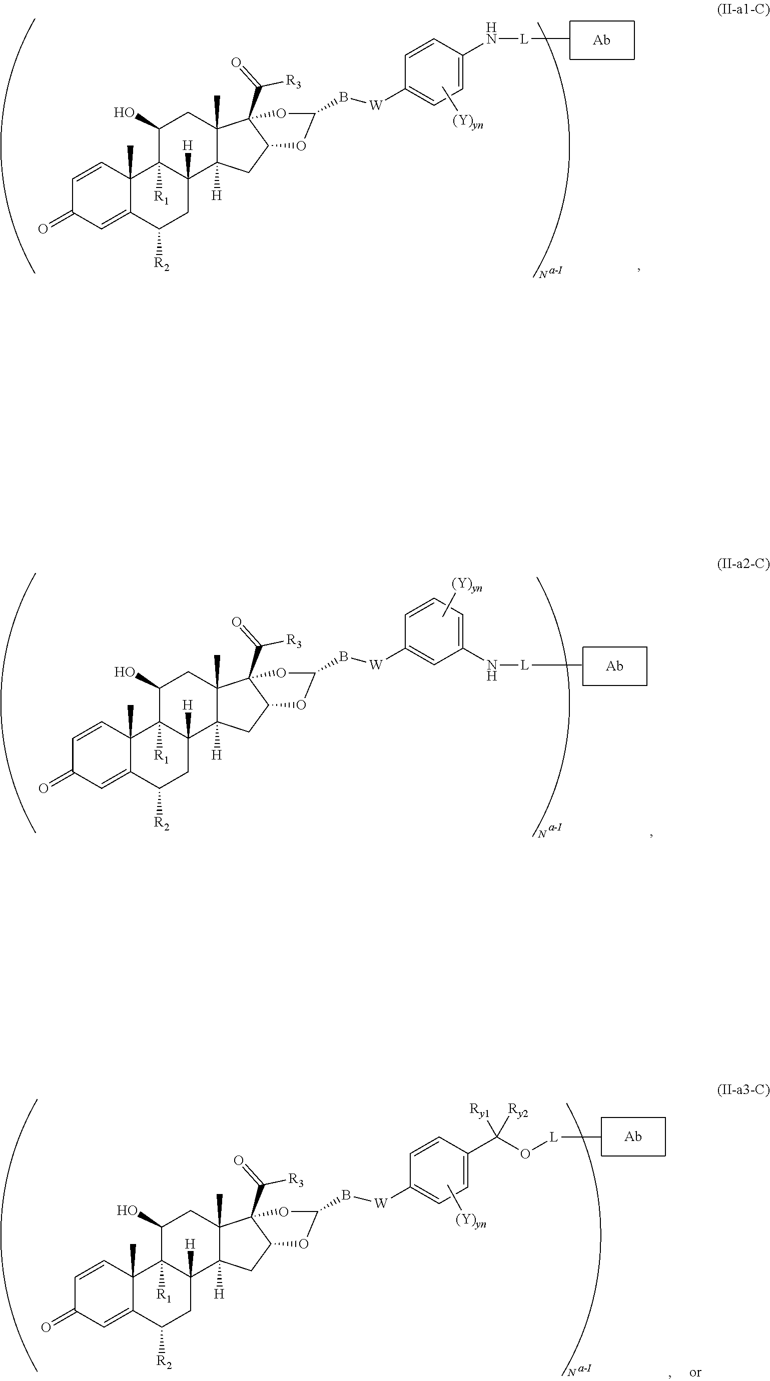

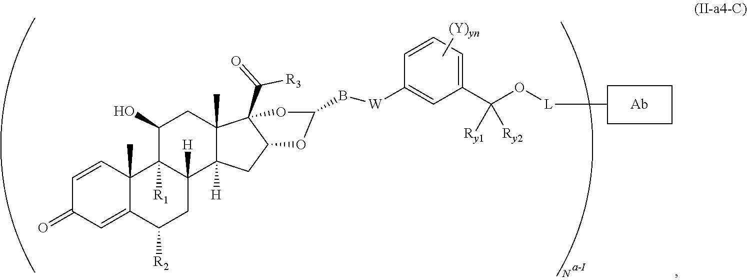

wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; L is absent or is any group; Ab is a ligand; N^{a-I} is a number equal to or greater than 0.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-D):

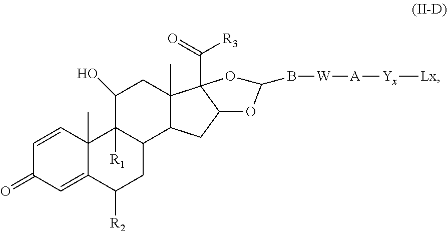

wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of —O—, optionally substituted —CH₂—O—, —S—, optionally substituted —PH—, optionally substituted —P(═O)H—, and optionally substituted —NH—; Lx is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-D), formula (II-a2-D), formula (II-a3-D), or formula (II-a4-D):

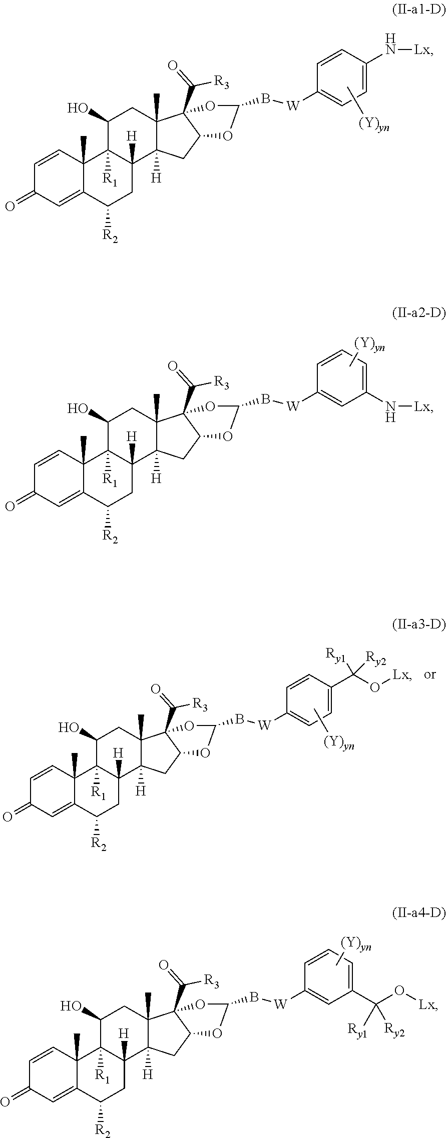

wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; Lx is any group.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure described herein.

In one aspect, the present application provides a compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

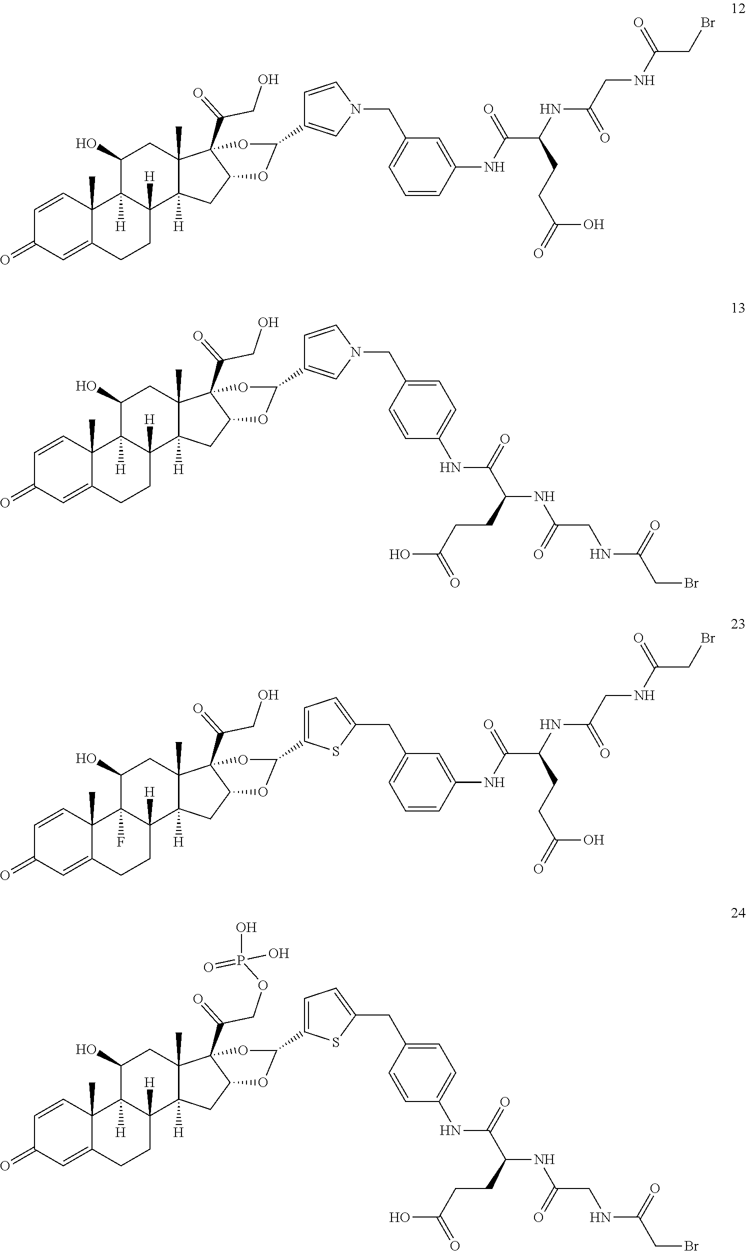

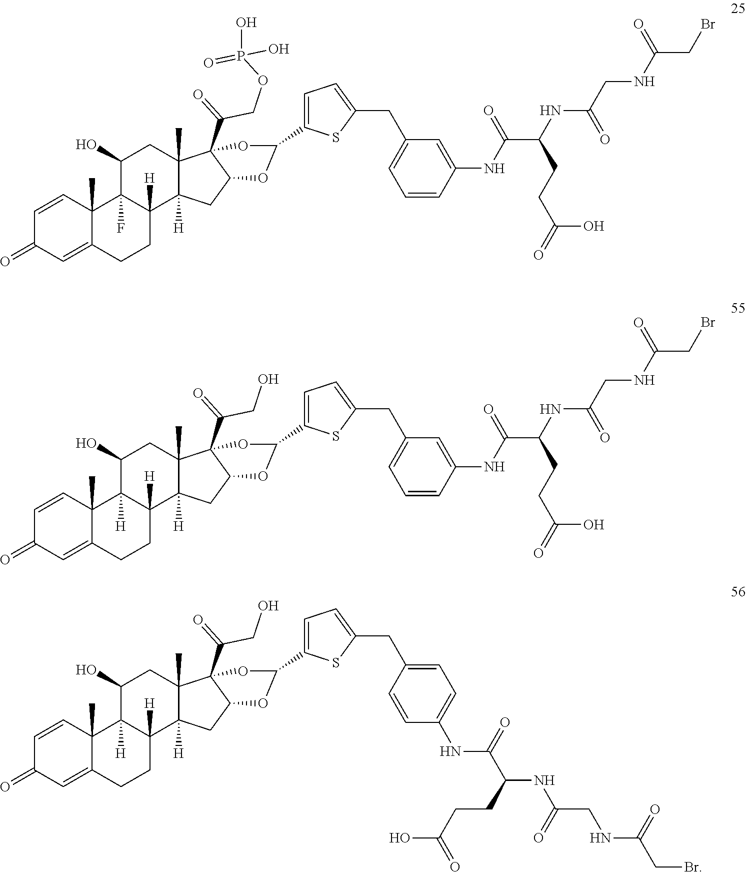

In one aspect, the present application provides a compound or a tautomer, a mesomer, a racemate, an enantiomer or a diastereoisomer thereof, or a mixture thereof, or a pharmaceutically acceptable salt thereof, wherein the compound comprises formula (II-a1-C-1), (II-a1-C-2), (II-a1-C-3), (II-a2-C-1), (II-a2-C-2), and (II-a2-C-3),

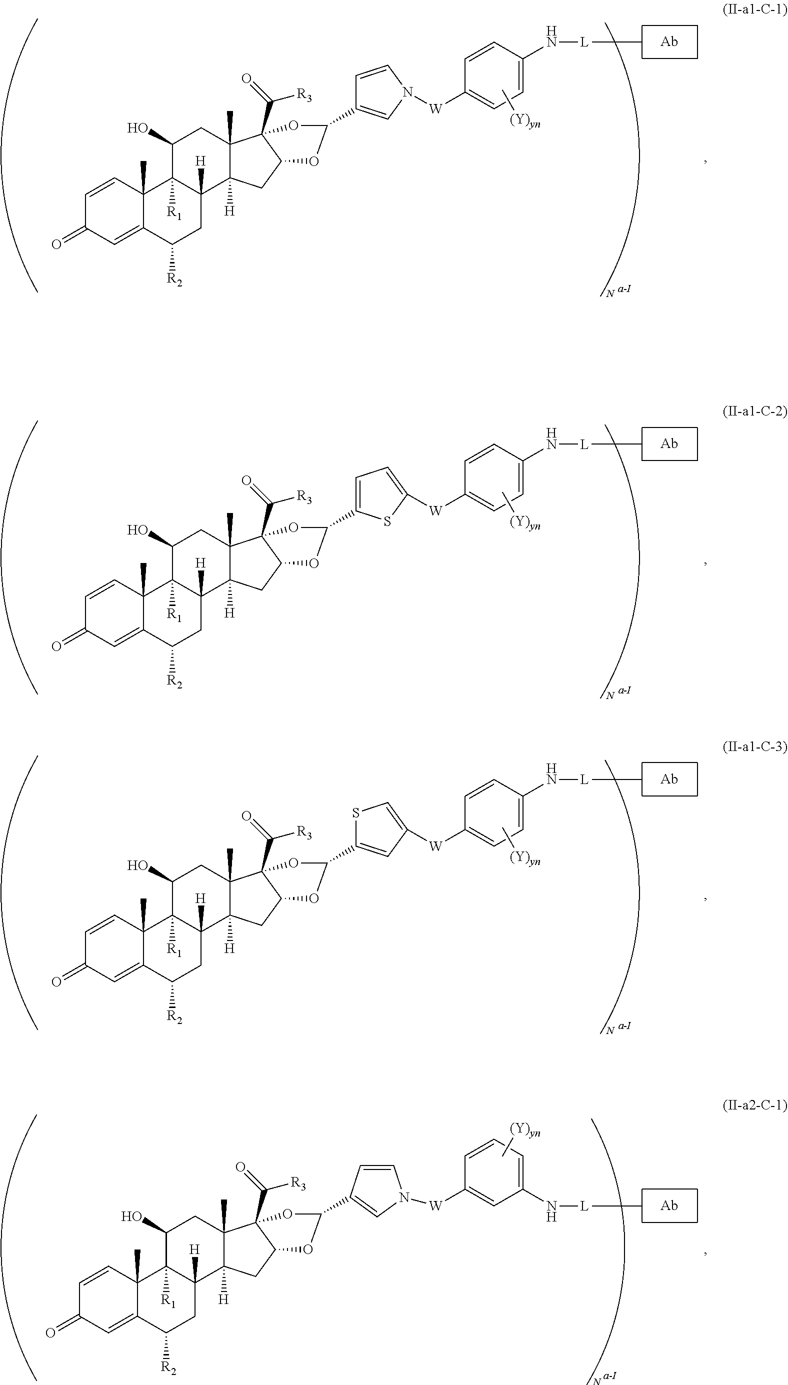

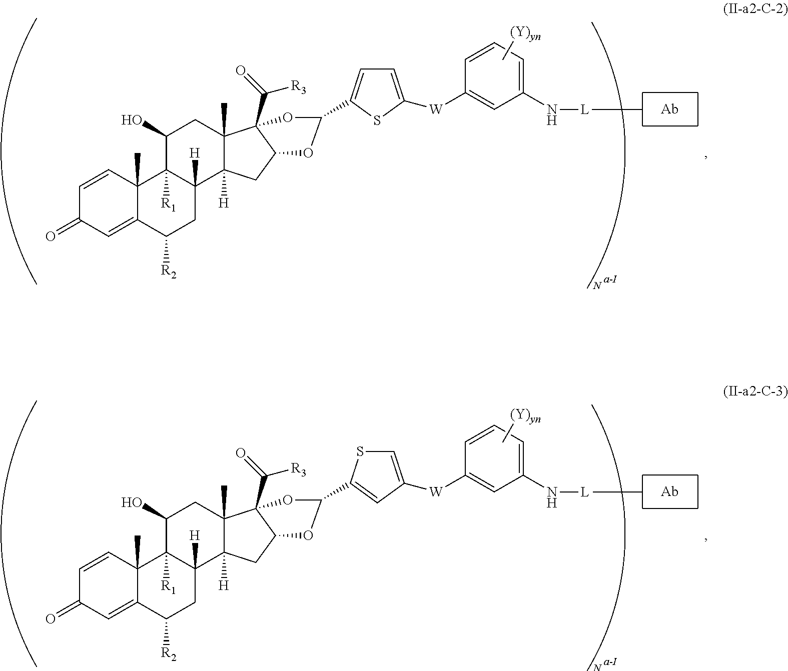

wherein,
R₁ and R₂ are each independently selected from: hydrogen, protium, deuterium, tritium, halogen, and C₁-C₆ alkyl;
R₃ is selected from: —CH₂Cl, —CH₂SH, —CH₂OH,

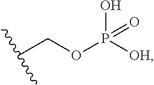 [SMILES: *COP(=O)(O)O]

—OCH₃, —OCH₂F, —OCH₂Cl, —OCH₂CN, —OCH₂CH₃, —SH, —SCH₂F, —SCH₂Cl, —SCH₂CF₃ and —SCH₂CN;
Y is each independently selected from: hydrogen, halogen, hydroxy, sulfhydryl, amino, C₁-C₆ alkyl, —OC₁-C₆ alkyl and —C₁-C₆ alkylhydroxy;
W is selected from

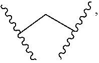 [SMILES: *C*]

### CH(CH₃)—, and

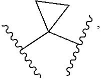 [SMILES: *C1(*)CC1]

N^{a-1} is a number from about 0 to about 20;
Ab is selected from an antibody or an antigen-binding fragment thereof;
yn is selected from 0, 1 or 2.

In another aspect, the present application provides a pharmaceutical composition comprising the compound or the tautomer, the mesomer, the racemate, the enantiomer or the diastereoisomer thereof, or the mixture thereof, or the pharmaceutically acceptable salt thereof described herein and/or the conjugate described herein, and optionally a pharmaceutically acceptable carrier.

In another aspect, the present application provides a method for influencing immune system functions, which comprises administering to a subject the compound or the tautomer, the mesomer, the racemate, the enantiomer or the diastereoisomer thereof, or the mixture thereof, or the pharmaceutically acceptable salt thereof described herein, the conjugate described herein and/or the pharmaceutical composition described herein.

In another aspect, the present application provides use of the compound or the tautomer, the mesomer, the racemate, the enantiomer or the diastereoisomer thereof, or the mixture thereof, or the pharmaceutically acceptable salt thereof described herein, the conjugate described herein and/or the pharmaceutical composition described herein in preparing a medicament for preventing and/or treating a disease and/or condition.

In another aspect, the present application provides the compound or the tautomer, the mesomer, the racemate, the enantiomer or the diastereoisomer thereof, or the mixture thereof, or the pharmaceutically acceptable salt thereof described herein, the conjugate described herein and/or the pharmaceutical composition described herein for use in the prevention and/or treatment of a disease and/or condition.

In another aspect, the present application provides a method for preventing and/or treating a disease and/or condition, which comprises administering to a subject in need the compound or the tautomer, the mesomer, the racemate, the enantiomer or the diastereoisomer thereof, or the mixture thereof, or the pharmaceutically acceptable salt thereof described herein, the conjugate described herein and/or the pharmaceutical composition described herein.

Other aspects and advantages of the present application will be readily apparent to those skilled in the art from the following detailed description. Only exemplary embodiments of the present application have been shown and described in the following detailed description. As those skilled in the art will recognize, the content of the present application enables those skilled in the art to make changes to the specific embodiments disclosed without departing from the spirit and scope of the invention to which the present application pertains. Accordingly, descriptions in the specification are only illustrative rather than restrictive.

### BRIEF DESCRIPTION OF THE DRAWINGS

Specific features of the invention to which the present application pertains are set forth in appended claims. Features and advantages of the invention to which the present application pertains will be better understood by reference to the exemplary embodiments and drawings described in detail below. The drawings are briefly described as follows:

FIGS. 1a-1b show the inhibitory effects of the compound of the present application on R848-induced release of IFNα and TNFα from human peripheral blood monocytes.

FIG. 2 shows the results of the effect of the compound of the present application on the biological activity in fluorescein isothiocyanate (FITC)-induced delayed-type IV hypersensitivity mouse models.

FIGS. 3a-3c show the results of the effect of the compound of the present application on the biological activity in bovine type II collagen mixed adjuvant-induced DBA/1 mouse arthritis models.

### DETAILED DESCRIPTION

The embodiments of the present invention are described below with reference to specific examples, and other advantages and effects of the present invention will be readily apparent to those skilled in the art from the disclosure of the present specification.

### Definitions of Terms

In the present application, the term “halogen” generally refers to fluorine, chlorine, bromine or iodine, and it may be, for example, fluorine or chlorine.

In the present application, the term “alkyl” generally refers to a residue derived from an alkane by removal of a hydrogen atom. Alkyl may be substituted or unsubstituted, or replaced or unreplaced. The term “alkyl” generally refers to a saturated linear or branched aliphatic hydrocarbon group having a residue derived from the parent alkane by removal of hydrogen atoms from the same carbon atom or two different carbon atoms, and it may be a linear or branched group containing 1 to 20 carbon atoms, e.g., 1 to 12 carbon atoms, such as alkyl containing 1 to 6 carbon atoms. Non-limiting examples of alkyl include but are not limited to methyl, ethyl, propyl, propyl, butyl, etc. Alkyl may be substituted or unsubstituted, or replaced or unreplaced. For example, when alkyl is substituted, substitution with a substituent may be performed at any available linking site, and the substituent may be independently optionally selected from one or more of alkyl, alkenyl, alkynyl, alkoxy, alkylthio, alkylamino, halogen, sulfhydryl, hydroxy, nitro, cyano, cycloalkyl, heterocyclyl, aryl, heteroaryl, cycloalkoxy, heterocycloalkoxy, cycloalkylthio, heterocycloalkylthio, and oxo, and the substituent may, e.g., be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group.

In the present application, the term “alkylene” generally refers to a saturated linear or branched aliphatic hydrocarbon group having 2 residues derived from the parent alkane by removal of two hydrogen atoms from the same carbon atom or two different carbon atoms, and it may be a linear or branched group containing 1 to 20 carbon atoms; for example, the term “methylene” may refer to a residue derived from a one-carbon atom group by removal of two hydrogen atoms. Methylene may be substituted or unsubstituted, or replaced or unreplaced; for example, alkylene contains 1 to 12 carbon atoms, e.g., alkylene containing 1 to 6 carbon atoms. Non-limiting examples of alkylene include but are not limited to methylene(—CH₂—), 1,1-ethylidene(—CH(CH₃)—), 1,2-ethylidene(—CH₂CH₂)—, 1,1-propylidene(—CH(CH₂CH₃)—), 1,2-propylidene(—CH₂CH(CH₃)—), 1,3-propylidene(—CH₂CH₂CH₂—), 1,4-butylidene(—CH₂CH₂CH₂CH₂—), 1,5-butylidene(—CH₂CH₂CH₂CH₂CH₂—), etc. Alkylene may be substituted or unsubstituted, or replaced or unreplaced. For example, when alkylene is substituted, substitution with a substituent may be performed at any available linking site, and the substituent is preferably independently optionally selected from one or more of alkyl, alkenyl, alkynyl, alkoxy, alkylthio, alkylamino, halogen, sulfhydryl, hydroxy, nitro, cyano, cycloalkyl, heterocyclyl, aryl, heteroaryl, cycloalkoxy, heterocycloalkoxy, cycloalkylthio, heterocycloalkylthio, and oxo, and the substituent may, e.g., be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group. Methylene or alkylene may be substituted or unsubstituted.

In the present application, the term “alkenyl” generally refers to a linear or branched hydrocarbon group containing one or more double bonds. Exemplary examples of alkenyl include allyl, homoallyl, vinyl, crotyl, butenyl, pentenyl, hexenyl, etc. Exemplary examples of C2-6 alkenyl containing no less than one double bond include butadienyl, pentadienyl, hexadienyl, and hexatrienyl, as well as branched forms thereof. The positions of the unsaturated bonds (double bonds) may be any positions in the carbon chain. Alkenyl may be substituted or unsubstituted.

In the present application, the term “alkenylene” generally refers to a group having a residue derived from an alkene by removal of two hydrogen atoms from a carbon atom. For example, alkenylene may be acrol, vinylene, butenylene, pentenylene, hexenylene, etc. Alkenylene may be substituted or unsubstituted.

In the present application, the term “alkynyl” generally refers to unsaturated linear or branched alkynyl, e.g., ethynyl, 1-propynyl, propargyl, or butynyl. Alkynyl may be substituted or unsubstituted.

In the present application, the term “alkynylene” generally refers to a group having a residue derived from an alkyne by removal of two hydrogen atoms from a carbon atom. For example, alkynylene may be ethynylene, propynylene, propargylene, butynylene, etc. Alkynylene may be substituted or unsubstituted.

In the present application, the term “aryl” generally refers to a group having a residue derived from an aromatic ring by removal of a hydrogen atom. The term “aromatic ring” may refer to a 6-14 membered all-carbon monocyclic ring or fused polycyclic ring (i.e., rings that share adjacent pairs of carbon atoms) having a conjugated 7-electron system, and it may be 6-10 membered, such as benzene and naphthalene. The aromatic ring can be fused to a heteroaryl, heterocyclyl or cycloalkyl ring, wherein the ring connected to the parent structure is the aryl ring. Aryl may be substituted or unsubstituted, and when it is substituted, the substituent may be one or more of the following groups independently selected from the group consisting of: alkyl, alkenyl, alkynyl, alkoxy, alkylthio, alkylamino, halogen, sulfhydryl, hydroxy, nitro, cyano, cycloalkyl, heterocycloalkyl, aryl, heteroaryl, cycloalkoxy, heterocycloalkoxy, cycloalkylthio and heterocycloalkylthio. Aryl may be substituted or unsubstituted.

In the present application, the term “arylene” generally refers to a group having a residue derived from an aromatic ring by removal of two hydrogen atoms from a carbon atom. For example, arylene may be phenylene and naphthylene. Arylene may be substituted or unsubstituted.

In the present application, the term “heteroaryl” generally refers to a group having a residue derived from a heteroaromatic ring by removal of a hydrogen atom from a carbon atom. The term “heteroaromatic ring” refers to a heteroaromatic system comprising 1 to 4 heteroatoms and 5 to 14 ring atoms, wherein the heteroatoms may be selected from the group consisting of oxygen, sulfur and nitrogen. Heteroaryl may be 5-10 membered and may be 5- or 6-membered, such as furanyl, thienyl, pyridinyl, pyrrolyl, N-alkylpyrrolyl, pyrimidinyl, pyrazinyl, imidazolyl and tetrazolyl. The heteroaryl ring can be fused to an aryl, heterocyclyl or cycloalkyl ring, wherein the ring connected to the parent structure is the heteroaryl ring. Heteroaryl may be optionally substituted or unsubstituted, and when it is substituted, the substituent may be one or more of the following groups independently selected from the group consisting of alkyl, alkenyl, alkynyl, alkoxy, alkylthio, alkylamino, halogen, sulfhydryl, hydroxy, nitro, cyano, cycloalkyl, heterocycloalkyl, aryl, heteroaryl, cycloalkoxy, heterocycloalkoxy, cycloalkylthio and heterocycloalkylthio. Heteroaryl may be substituted or unsubstituted.

In the present application, the term “heteroarylene” generally refers to a group having a residue derived from a heteroaromatic ring by removal of two hydrogen atoms from a carbon atom. For example, heteroarylene may be furylene, thienylene, pyridylidene, pyrrolylene, pyrimidinylene, pyrazinylene, imidazolylene, tetrazolylene, etc. Heteroarylene may be substituted or unsubstituted.

In the present application, the term “alcyl” generally refers to a group having a residue derived from an aliphatic ring by removal of a hydrogen atom from the same carbon atom or from a plurality of different carbon atoms. The term “cycloalkane” generally refers to a saturated or partially unsaturated monocyclic or polycyclic cyclic hydrocarbon, and the carbon ring contains 3 to 20 carbon atoms, may contain 3 to 12 carbon atoms, may contain 3 to 10 carbon atoms, and may contain 3 to 8 carbon atoms. Non-limiting examples of alcyl include cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic carbon rings may include spiro, fused and bridged carbon rings. Alcyl may be substituted or unsubstituted. In the present application, the term “carbocyclyl” generally refers to a group having a residue derived from a carbon ring by removal of a hydrogen atom from a carbon atom. The term “carbon ring” generally refers to a saturated or partially unsaturated monocyclic or polycyclic cyclic hydrocarbon, and it contains 3 to 20 carbon atoms, may contain 3 to 12 carbon atoms, may contain 3 to 10 carbon atoms, and may contain 3 to 8 carbon atoms. Non-limiting examples of a monocyclic carbon ring include cyclopropane, cyclobutane, cyclopentane, cyclopentene, cyclohexane, cyclohexene, cyclohexadiene, cycloheptane, cycloheptatriene, cyclooctane, etc.; polycyclic carbon rings may include spiro, fused and bridged carbon rings. Carbocyclyl may be substituted or unsubstituted. In some instances, alicyclic and carbocyclic rings may be used in place of one another.

In the present application, the term “partially unsaturated” generally means that the cyclic structure contains at least one double or triple bond between the ring molecules. The term “partially unsaturated” encompasses cyclic structures having multiple sites of unsaturation, but is not intended to include aromatic or heteroaromatic rings defined herein. The term “unsaturated” means that the moiety has one or more degrees of unsaturation.

In the present application, the term “alcylene” generally refers to a group having a residue derived from an alicyclic ring by removal of two hydrogen atoms from a carbon atom. For example, alcylene may be cyclopropylene, cyclobutylene, cyclopentylene, cyclopentenylene, cyclohexylene, cyclohexenylene, cyclohexadienylene, cycloheptylene, cycloheptatrienylene, cyclooctylene, etc.; polycyclic carbon rings may include spiro, fused and bridged carbon rings. Alcylene may be substituted or unsubstituted.

In the present application, the term “aliphatic heterocyclyl” generally refers to a 3-7 membered monocyclic carbon ring structure, a fused 7-10 membered bicyclic heterocyclic structure or a bridged 6-10 membered bicyclic heterocyclic structure that is stable and non-aromatic. These cyclic structures may be saturated or partially saturated and also contain one or more heteroatoms in addition to carbon atoms, wherein the heteroatoms may be selected from the group consisting of: oxygen, sulfur and nitrogen. For example, they contain 1 to 4 heteroatoms as defined above. When used to refer to atoms on an aliphatic heterocyclic cyclic structure, the term “nitrogen” may include nitrogen that undergoes a substitution reaction. For example, the aliphatic heterocyclyl may comprise “heterocycloalkyl” that may refer to a 3-7 membered monocyclic alkyl structure, a fused 7-10 membered bicyclic heterocyclic structure or a bridged 6-10 membered bicyclic heterocyclic structure that is stable and non-aromatic. These cyclic structures also contain one or more heteroatoms in addition to carbon atoms, wherein the heteroatoms may be selected from the group consisting of oxygen, sulfur and nitrogen. For example, they contain 1 to 4 heteroatoms as defined above. Heterocycloalkyl may be substituted or unsubstituted. Aliphatic heterocyclyl may be substituted or unsubstituted.

In the present application, the term “aliphatic heterocyclylene” generally refers to a group having a residue derived from an alicyclic ring by removal of two hydrogen atoms from a carbon atom. Aliphatic heterocyclylene may be substituted or unsubstituted.

In the present application, the term “ring-forming atom” generally refers to an atom contained in a cyclic structure. For example, a ring-forming atom may be a carbon atom in a benzene ring or may be a nitrogen atom in a pyridine ring. When a hydrogen atom is linked to a ring-forming atom, the ring-forming atom may be substituted or unsubstituted.

In the present application, the term “each independently” generally means that a variable applies in any case irrespective of the presence or absence of variables having the same or different definitions in the same compound. For example, the variable may refer to the type or number of substituents in the compound, the type of atoms in the compound, and so on. For example, where R occurs twice in a compound and R is defined as “independently carbon or nitrogen”, both R can be carbon, both R can be nitrogen, or one R can be carbon and the other R is nitrogen.

In the present application, the term “optional” or “optionally” generally means that the event or circumstance subsequently described may, but not necessarily occur, and that the description includes instances where the event or circumstance occurs or does not occur. For example, “heterocyclyl group optionally substituted with alkyl” means that alkyl may be, but not necessarily present, and that the description may include instances where the heterocyclyl group is or is not substituted with alkyl.

In the present application, the term “substituted” generally means that one or more hydrogen atoms in the group, for example, up to 5 (e.g., 1 to 3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the term “0 or more (e.g., 0 or 1 or more, 0 or 1, or 0) methylene units are replaced” generally means that when the structure comprises one or more methylene units, the one or more methylene units may be unreplaced, or may be replaced by one or more groups that are not methylene (e.g., —NHC(O)—, —C(O)NH—, —C(O)—, —OC(O)—, —C(O)O—, —NH—, —O—, —S—, —SO—, —SO₂—, —PH—, —P(═O)H—, —NHSO₂—, —SO₂NH—, —C(═S)—, —C(═NH)—, —N═N—, —C═N—, —N═C— or —C(═N₂)—).

In the present application, the term “group capable of coupling with amino” generally means that the compound A has amino, the compound B has a group capable of coupling with amino, and the compound B can be linked to the compound A by reacting with amino of the compound A via the group capable of coupling with amino.

In the present application, the term “group capable of coupling with sulfhydryl” generally means that the compound A has sulfhydryl, the compound B has a group capable of coupling with sulfhydryl, and the compound B can be linked to the compound A by reacting with sulfhydryl of the compound A via the group capable of coupling with sulfhydryl.

In the present application, the term “click chemistry group” generally refers to a reactive group that is capable of rapid and efficient coupling. For example, click chemistry reactions may comprise the following group of reactions: cycloaddition reactions, nucleophilic ring-opening reactions, non-aldol carbonyl chemistry, and additions to carbon-carbon multiple bonds. For example, click chemistry groups may be selected from the group consisting of:

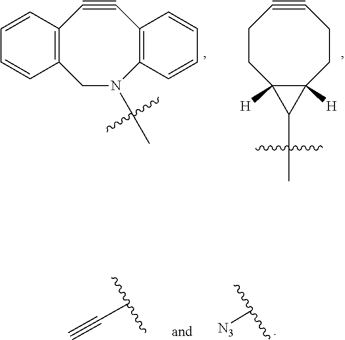

In the present application, the “linking” of group X to group Y may generally be in any orientation, which generally means that when group X is used for linker Y and group Z, two or more linking sites of the group X may be linked arbitrarily to either group Y or group Z. For example, when the —C(O)O— of SP² is linked to the —NH—CH₂— of (SP¹)ₘ₁, it is possible that the C atom of SP² is linked to the N atom of (SP¹)ₘ₁, the O atom of SP² is linked to the N atom of (SP¹)ₘ₁, the C atom of SP² is linked to the C atom of (SP¹)ₘ₁, or the O atom of SP² is linked to the C atom of (SP¹)_{m1.}

In the present application, the term “substituted” generally means that one or more hydrogen atoms in the group, for example, 1 to 3 hydrogen atoms, may be each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the term “compound” generally refers to a substance having two or more different elements. For example, the compound of the present application may be an organic compound. For example, the compound described herein may be a compound having a molecular weight of no more than 500, a compound having a molecular weight of no more than 1000, a compound having a molecular weight of no less than 1000, or a compound having a molecular weight of no less than 10,000, or no less than 100,000. In the present application, the compound may further refer to a compound that involves linking by a chemical bond, for example, a compound where one or more molecules having a molecular weight of no more than 1000 are linked, via a chemical bond, to a biological macromolecule, wherein the biological macromolecule may be polysaccharide, protein, nucleic acid, polypeptide, and the like. For example, the compound of the present application may include a compound where a protein is linked to one or more molecules having a molecular weight of no more than 1000, a compound where a protein is linked to one or more molecules having a molecular weight of no more than 10,000, or a compound where a protein is linked to one or more molecules having a molecular weight of no more than 100,000.

In the present application, the terms such as “alkyl”, “alkenyl” and “cycloalkyl” may be preceded by a notation to indicate the number of atoms present in the group under particular circumstances as in C₁-C₄ alkyl, C3-C7 cycloalkoxy, and C₁-C₄ alkylcarbonylamino and the like, as known to those skilled in the art, and the subscript numeral following “C” indicates the number of carbon atoms present in the group. For example, C₃ alkyl refers to an alkyl group containing three carbon atoms (e.g., n-propyl or isopropyl); in C₁₋₁₀, members of the group may contain any number of carbon atoms within the range of 1-10.

One or more hydrogen atoms in the group, for example, up to 5 (e.g., 1 to 3) hydrogen atoms, are each independently substituted with a corresponding number of substituents. A substituent is only in its possible chemical position, and those skilled in the art will be able to determine (by experiments or theories) possible or impossible substitution without undue efforts. For example, it may be unstable when amino or hydroxy having a free hydrogen is bound to a carbon atom having an unsaturated (such as olefin) bond.

In the present application, the term “hydrophilic amino acid” is generally determined based on the hydrophilicity of glycine and an amino acid having a higher hydrophilicity than that of glycine can be regarded as a hydrophilic amino acid. For example, a hydrophilic amino acid may comprise an amino acid selected from the group consisting of: serine (S), glutamine (Q), arginine (R), lysine (K), asparagine (N), glutamic acid (E), proline (P), and aspartic acid (D).

In the present application, the term “hydrophobic amino acid” is generally determined based on the hydrophilicity of glycine and an amino acid having a lower hydrophilicity than that of glycine can be regarded as a hydrophobic amino acid. For example, a hydrophobic amino acid may comprise an amino acid selected from the group consisting of phenylalanine (F), isoleucine (I), leucine (L), tryptophan (W), valine (V), methionine (M), tyrosine (Y), citrulline (C), alanine (a), threonine (T), and histidine (H). For example, glycine, as a special amino acid, is neither a hydrophilic amino acid nor a hydrophobic amino acid.

The pharmaceutical composition may be in the form of a sterile injectable aqueous or oily suspension for intramuscular and subcutaneous administration. The suspension may be prepared according to a known technique using those suitable dispersing agents or wetting agents and suspending agents described above. The sterile injectable formulation may also be a sterile injection or suspension prepared in a parenterally acceptable non-toxic diluent or solvent, e.g., a solution prepared in 1,3-butanediol. In addition, a sterile fixed oil may be conventionally used as a solvent or a suspending medium. For example, any blend fixed oil including synthetic mono- or di-glycerides can be used. In addition, fatty acids such as oleic acid may also be used in the preparation of injections.

The term “drug loading” generally refers to the average number of drugs loaded per ligand and may also be expressed as the ratio of drug to antibody, and the drug loading may range from 0 to 12 (e.g., 1 to 10) drugs per ligand (Ab). In the embodiments of the present application, the drug loading is expressed as N^{a-1}, and exemplary values may be an average of 1, 2, 3, 4, 5, 6, 7, 8, 9 and 10. The drug loading per ADC molecule after the coupling reaction can be characterized by conventional methods such as UV/visible spectroscopy, mass spectrometry, ELISA assays and HPLC. For example, the number of chemical bonds linked to a ligand of the present application may be not limited to 1, and when the present application uses one horizontal line to indicate that a ligand is linked to a drug or a ligand is linked to a linker, the horizontal line may indicate that the ligand is linked to the drug or the linker via one chemical bond, such as a covalent bond, and may also indicate that the ligand is linked to the drug or the linker via two or more chemical bonds, such as covalent bonds.

In the present application, the term “comprise” “comprising”, “contain” or “containing” is generally intended to include the explicitly specified features without excluding other elements. The terms “no less than” and “no more than” generally refer to the situations where the number itself is included.

In the present application, the term “about” generally means varying by 0.5%-10% above or below the stated value, for example, varying by 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5% or 10% above or below the stated value.

In the present application, the compound of the present application includes tautomers, mesomers, racemates, enantiomers and/or diastereoisomers thereof: In the present application, the term “diastereoisomer” generally refers to a stereoisomer that has two or more chiral centers and whose molecules are not mirror images of each other. Diastereoisomers may have different physical properties, e.g., melting points, boiling points, spectral properties, and reactivities. In the present application, the terms “tautomer” and “tautomeric form” are used interchangeably and generally refer to structural isomers of different energies that can be converted into each other by crossing a low energy barrier. For example, proton tautomers (also known as prototropic tautomers) include interconversions by proton migration, such as keto-enol isomerization and imine-enamine isomerization. Valence tautomers include interconversions by recombination of some bonding electrons. In the present application, the term “mesomer” generally means that the molecule contains asymmetric atoms but the total optical rotation is zero due to the presence of symmetric factors. The term “racemate” or “racemic mixture” refers to a composition of two enantiomeric substances in equimolar amounts.

In the present application, certain atoms of the compound of the present application may be present in no less than one isotopic form. For example, hydrogen may occur as protium (¹H), deuterium (²H), and tritium (H), and carbon may naturally occur as three different isotopes (¹²C, ¹³C, and “C). Examples of isotopes that can be incorporated into compound of the present application also include but are not limited to ¹⁵N, ¹⁸O, ¹⁷O, ¹⁸F, ³²P, ³³P, ¹²⁹I, ¹³¹I, ¹²³I, ¹²⁴I, ¹²⁵I, or similar isotopes. Thus, the compounds of the present application may be enriched with one or more of these isotopes relative to the natural abundance of these isotopes. Such isotopically enriched compounds can be used for a variety of purposes, as known to those skilled in the art. For example, replacement with heavy isotopes such as deuterium (²H) may offer certain therapeutic advantages, possibly due to higher metabolic stability. For example, the natural abundance of deuterium (²H) is about 0.015%. Accordingly, one out of about 6500 hydrogen atoms is a deuterium atom. Accordingly, the deuterium abundance of one or more sites (as the case may be) in the deuterium-containing compound of the present application is greater than 0.015%. Unless otherwise indicated, the structures described herein may also include compounds that differ only in the presence or absence of one or more isotopically enriched atoms. For example, compounds having a structure identical to the structure disclosed herein except for the substitution of the hydrogen atom with deuterium or tritium or the substitution of the carbon atom with carbon 13 or carbon 14 are within the scope of the present application.

In the present application, the term “pharmaceutical composition” generally refers to a mixture containing one or more of the compounds described herein or a physiologically/pharmaceutically acceptable salt or pro-drug thereof, and other chemical components, and other components, for example, physiologically/pharmaceutically acceptable carriers and excipients. The pharmaceutical composition may promote the administration to an organism, which facilitates the absorption of the active ingredient, thereby exerting biological activities. For preparation of conventional pharmaceutical compositions, reference can be made to Chinese Pharmacopoeia.

In the present application, the term “pharmaceutically acceptable salt” generally refers to a salt of a compound or ligand-drug conjugate of the present application, or a salt of a compound described herein. Such salts may be safe and/or effective when used in mammals and may possess the required biological activity, and the antibody-antibody drug conjugate compound of the present application may form a salt with an acid, and non-limiting examples of pharmaceutically acceptable salts include: hydrochloride, hydrobromide, hydriodate, sulphate, bisulfate, citrate, acetate, succinate, ascorbate, oxalate, nitrate, sorbate, hydrophosphate, dihydrophosphate, salicylate, hydrocitrate, tartrate, maleate, fumarate, formate, benzoate, mesylate, ethanesulfonate, benzenesulphonate andp-toluenesulfonate.

In the present application, the term “conjugate” generally refers to a compound prepared by subjecting the compounds of the present application to one or more chemical reactions or by linking the compounds to one another via one or more linking structures such as bridges, spacers, or linkers.

In the present application, the term “pharmaceutically acceptable carrier” generally refers to a carrier for providing therapeutic agents, such as antibodies or polypeptides, genes, and other therapeutic agents. The term refers to any pharmaceutical carrier that does not itself induce the production of antibodies harmful to the individual that receives the composition and can be given without producing undue toxicity. Suitable carriers may be macromolecules that are large and metabolize slowly, such as proteins, polysaccharides, polylactic acids, polyglycolic acids, poly(amino acid)s, amino acid copolymers, lipid aggregates, and inactivated virus particles. Such carriers are well known to those skilled in the art. Pharmaceutically acceptable carriers in therapeutic compositions may include liquids such as water, saline, glycerol, and ethanol. Auxiliary substances, such as wetting or emulsifying agents, or pH buffering substances, may also be present in these carriers.

In the present application, the term “antibody” generally refers to an immunoglobulin that is reactive to a specified protein or peptide or a fragment thereof: The antibody can be an antibody of any class, including but not limited to IgG, IgA, IgM, IgD and IgE, and of any subclass (e.g., IgG1, IgG2, IgG3 and IgG4). The antibody may have a heavy chain constant region selected from, for example, IgG1, IgG2, IgG3 or IgG4. The antibody may also have a light chain selected from, for example, kappa (κ) or lambda (λ). The antibodies of the present application may be derived from any species. The term “antibody” may comprise intact polyclonal antibodies, intact monoclonal antibodies, chimeric antibodies, humanized antibodies, human antibodies, fusion proteins comprising an antibody and any other modified immunoglobulin molecules so long as the antibodies exhibit the desired biological activity.

In the present application, the term “antigen-binding fragment” generally refers to a portion of an antibody molecule that comprises amino acids responsible for specific binding of an antibody to an antigen. The portion of the antigen specifically recognized and bound by the antibody is referred to as the “epitope” described above. As described above, the antigen-binding domain may typically comprise an antibody light chain variable region (VL) and an antibody heavy chain variable region (VH); however, the antigen-binding domain does not necessarily comprise both. A Fd fragment, for example, has two VH regions and typically retains some of the antigen-binding functions of the intact antigen-binding domain. Examples of antigen-binding fragments of antibodies include (1) a Fab fragment, a monovalent fragment having a VL, a VH, a constant light chain (CL) and a CH1 domain; (2) an F(ab′)₂ fragment, a bivalent fragment having two Fab fragments linked by a disulfide bridge at the hinge region; (3) an Fd fragment having two VH and CH1 domains; (4) an Fv fragment having VL and VH domains of a single arm of an antibody, (5) a dAb fragment having a VH domain; (6) an isolated complementarity determining region (CDR); (7) a single chain Fv (scFv), e.g., derived from a scFV-library; although the two domains of the Fv fragment, VL and VH, are encoded by separate genes, they may be joined by a recombinant method using a synthetic linker that allows them to be prepared as a single protein chain in which the VL and VH regions pair to form monovalent molecules (referred to as single chain Fv (scFv)); and (8) VHH, which relates to variable antigen-binding domains of heavy chain antibodies from Camelidae (camel, dromedary, llama, alpaca, etc.). VHH may also be referred to as nanobody (Nb).

In the present application, the term “variable region” or “variable domain” generally refers to a domain of a heavy chain or light chain of an antibody involved in the binding of the antibody to an antigen. In the present application, the term “variable” generally means that certain portions of the sequences of the variable domains of antibodies vary considerably, resulting in the binding and specificity of various particular antibodies to their particular antigens. Variability is not evenly distributed throughout the variable regions of the antibody. It is concentrated in three segments in each of the light chain and heavy chain variable regions called complementarity determining regions (CDRs) or hypervariable regions (HVRs), which are LCDR1, LCDR2, LCDR3, HCDR1, HCDR2 and HCDR3. The more highly conserved portions of the variable domains are called frameworks (FRs). The variable domains of native heavy and light chains each comprise four FRs (H—FR1, H—FR2, H—FR3, H—FR4, L-FR1, L-FR2, L-FR3 and L-FR4) largely in a β-sheet configuration. The FRs are connected by three CDR structural loop regions. The CDRs in each chain are held in close proximity by the FR regions and form, together with the CDRs from the other chain, the antigen-binding sites of the antibody. In the art, the variable regions of an antibody can be encoded or the CDRs of an antibody can be divided by various methods, such as Kabat numbering scheme and definition rule based on sequence variability, Chothia numbering scheme and definition rule based on the regional position of a structural loop, IMGT numbering scheme and definition rule based on alignment of amino acid sequences of germline V genes proposed by efranc et al., as well as Honneger's numbering scheme (AHo's), Martin numbering scheme, Gelfand numbering scheme, and the like.

In the present application, the term “percent (%) sequence identity” generally refers to the number of matches (“hits”) of identical amino acids of two or more aligned amino acid sequences as compared to the number of amino acid residues making up the overall length of the amino acid sequences. In other words, using an alignment, for two or more sequences, the percent of amino acid residues that are the same (e.g., 70%, 75%, 80%, 85%, 90%, 95%, 96%, 97%, 98% or 99% sequence identity) can be determined when the sequences are compared and aligned for maximum correspondence (as measured using a sequence comparison algorithm known in the art) or when manually aligned and visually inspected. Thus, the sequences that are compared to determine sequence identity may differ by one or more substitutions, additions or deletions of amino acids. Suitable programs for aligning protein sequences are known to those skilled in the art. The percent sequence identity of protein sequences can, for example, be determined with programs such as CLUSTALW, Clustal Omega, FASTA, or BLAST, e.g., using the NCBI BLAST algorithm.

In the present application, the term “antibody analog” is generally used in the broadest sense and specifically covers a molecule that specifically binds to a target molecule with monospecificity and that is structurally different from a native antibody. For example, the term “antibody analog” refers to an antibody that comprises a segment having substantial identity to a portion of an amino acid sequence and has at least one of the following properties: (1) specific binding to the same antigen under appropriate binding conditions, and (2) ability to inhibit at least one biological activity of the antigen. Typically, antibody analogs comprise a conservative amino acid substitution (or insertion or deletion) with respect to the native sequence. Analogs typically have a length of at least 20 or 25 amino acids, at least 50, 60, 70, 80, 90, 100, 150 or 200 amino acids or more amino acids, and can generally be as long as a full-length heavy chain or light chain of the antibody. Some examples include antibody analogs with 1, 2, 3, 4, 5, 6, 7, 8, 9, 10, 11, 12, 13, 14, 15, 16 or 17 substitutions as compared to the germline amino acid sequence.

In the present application, the term “BDCA2” generally refers to a type II C-type lectin. The BDCA2 of the present application may generally comprise blood dendritic cell antigen 2, variants thereof, and functionally active fragments thereof. Using BDCA-2 as a target molecule may enable targeting of the inflammatory environment of dendritic cells. For example, the accession number for BDCA2 in UniProt may be Q8WTT0.

In the present application, the term “TNFα” generally refers to a cytokine such as tumor necrosis factor-a. The TNFα of the present application may generally include tumor necrosis factor-a, variants thereof, and functionally active fragments thereof: Using TNFα as a target molecule may enable targeting of the inflammatory environment. For example, the accession number for TNFα in UniProt may be P01375.

In the present application, the term “CD40” generally refers to the 50-55 kDa transmembrane glycoprotein of the tumor necrosis factor (TNF) receptor family. The CD40 of the present application may generally comprise CD40, variants thereof, and functionally active fragments thereof. Using CD40 as a target molecule may enable targeting of the inflammatory environment of B cells. For example, the accession number for CD40 in UniProt may be A0A0S2Z3C7.

In the present application, the term “IFNAR” generally refers to an interferon receptor. The IFNAR of the present application may generally comprise an interferon receptor, variants thereof, and functionally active fragments thereof. Using IFNAR as a target molecule may enable targeting of the inflammatory environment of immune cells. For example, the accession number for IFNAR in UniProt may be P17181.

In the present application, the term “effective amount” or “therapeutically effective amount” generally refers to an amount of the compound or pharmaceutical composition described herein sufficient to achieve the intended use described below, including but not limited to disease treatment. The therapeutically effective amount may vary according to: intended use (in vivo or in vitro); the subject and disease condition being treated, e.g., the weight and age of the subject, the severity of the disease condition; the mode of administration and the like, which can readily be determined by one of ordinary skill in the art. The term also applies to a dose that will induce a particular response in the target cells. The specific dose will vary depending on, for example, the particular compound chosen, the dosing regimen to be followed, whether being administered in combination with other agents, time of administration, the tissue to which it is administered, and the physical delivery system in which it is carried.

In the present application, the term “in vivo” generally refers to an event that occurs in a subject's body. In the present application, the term “in vitro” generally refers to an event that occurs outside a subject's body. For example, in vitro assays include any assay conducted outside of a subject. In vitro assays include cell-based assays in which cells, alive or dead, are employed. In vitro assays also include a cell-free assay in which no intact cells are employed.

In the present application, the terms “treatment” and “treating” generally refer to a method for achieving beneficial or desired results, including but not limited to therapeutic benefits. Therapeutic benefits include but are not limited to eradication, inhibition, reduction, or amelioration of the underlying disorder being treated. In addition, therapeutic benefits are achieved by eradicating, inhibiting, reducing, or ameliorating one or more physiological symptoms associated with the underlying disorder, and thus improvements are observed in the patient, but the patient may still be afflicted with the underlying disorder.

In the present application, the terms “prevention” and “preventing” generally refer to a method for achieving beneficial or desired results, including but not limited to prophylactic benefits. For the purpose of prophylactic benefits, a pharmaceutical composition can be administered to a patient at risk of developing a particular disease, or to a patient who reports he/she has one or more physiological symptoms of the disease, even if the patient has not yet been diagnosed with the disease.

In the present application, the term “subject” or “patient” generally refers to a human (i.e., a male or female in any age group, e.g., a pediatric subject (e.g., an infant, a child, or an adolescent) or an adult subject (e.g., a young adult, a middle-aged adult or a senior adult)) and/or other primate (e.g., a cynomolgus monkey or a rhesus monkey); a mammal, including commercially relevant mammals, such as cows, pigs, horses, sheep, goats, cats and/or dogs; and/or birds, including commercially relevant birds such as chickens, ducks, geese, quail and/or turkeys.

In the present application, the term “about” or “approximately” generally refers to an acceptable error of a particular value as determined by one of ordinary skill in the art, which depends in part on how the value is measured or determined. In certain embodiments, the term “about” or “approximately” generally means within 1, 2, 3 or 4 standard deviations. In certain embodiments, the term “about” or “approximately” generally means within 50%, 20%, 15%, 10%, 9%, 8%, 7%, 6%, 5%, 4%, 3%, 2%, 1%, 0.5%, or 0.05% of a given value or range.

In the present application, the term “comprise” and its variants, including, “comprises”, “comprising” and other forms, generally mean being inclusive of other components, elements, integers, steps and the like.

### DETAILED DESCRIPTION OF THE INVENTION

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I):

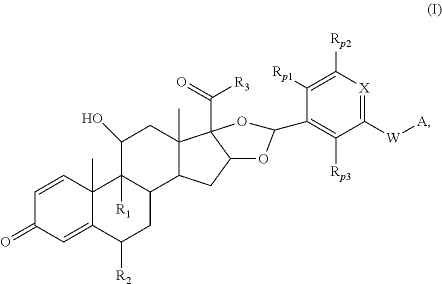

wherein
R₁, R₂, R₃, Rₚ₁, Rₚ₂, and Rₚ₃ may each independently be any group;
X may be selected from the group consisting of N and optionally substituted CH;
W may be absent or W may be any group;
A may be any group, wherein A may further be substituted with one or more Y, and each Y may be independently selected from the group consisting of hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂; when X may be CH and W may be —S—, A may not be phenyl.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1) or formula (I-a2):

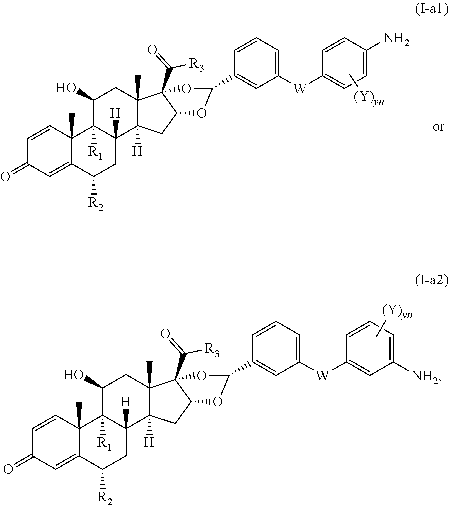

wherein
R₁, R₂, R₃, and Y may each independently be any group, yn may be a number equal to or greater than 0, and
W may be absent or W may be any group, wherein W may not be —S—.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₁, R₂ and R₃ may each independently be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substitute alkynyl, optionally substituted alcyl, optionally substituted aliphatic heterocyclyl, optionally substituted aryl, and optionally substituted heteroaryl; wherein, when R₁, R₂, and R₃ comprise methylene units, the methylene units of the R₁, R₂, and R₃ may each independently be unreplaced, or the methylene units of the R₁, R₂, and R₃ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene, and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein R₁ may be selected from the group consisting of: hydrogen, protium, deuterium, tritium, halogen, and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein R₁ may be selected from the group consisting of: hydrogen, F, Cl, Br, and optionally substituted methyl. For example, R₁ may be H or fluorine.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₂ may be selected from the group consisting of: hydrogen, protium, deuterium, tritium, halogen, and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₂ may be selected from the group consisting of: hydrogen, F, Cl, Br, and optionally substituted methyl. For example, R₂ may be H or fluorine.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃ may be selected from the group consisting of hydrogen, optionally substituted —OH, optionally substituted —SH, and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when R₃ may be optionally substituted methyl, R₃ may be substituted with R₃₁, and R₃₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₁ comprises a methylene unit, the methylene unit of R₃₁ may each independently be unreplaced, or the methylene unit of R₃₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₁ may be selected from the group consisting of: hydrogen, halogen, optionally substituted —OH, and optionally substituted —SH.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when R₃₁ may be optionally substituted —OH, R₃₁ may be substituted with R₃₁₁, and R₃₁₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₁₁ comprises a methylene unit, the methylene unit of R₃₁₁ may each independently be unreplaced, or the methylene unit of R₃₁₁ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₁₁ may be selected from the group consisting of: hydrogen, optionally substituted —P(═O)H₂, optionally substituted C₁-C₆ alkyl, and optionally substituted C₂-C₉ aliphatic heterocyclylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein R₃₁₁ may be selected from the group consisting of hydrogen, optionally substituted

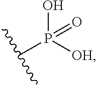 [SMILES: *P(=O)(O)O]

optionally substituted

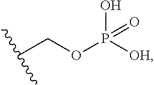 [SMILES: *COP(=O)(O)O]

and optionally substituted

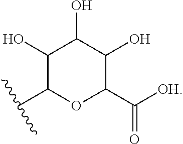 [SMILES: *C1OC(C(=O)O)C(O)C(O)C1O]

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when R₃ may be optionally substituted —OH, R₃ may be substituted with R₃₂, and R₃₂ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₂ comprises a methylene unit, the methylene unit of R₃₂ may each independently be unreplaced, or the methylene unit of R₃₂ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₂ may be selected from the group consisting of hydrogen, and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when R₃₂ may be optionally substituted methyl or optionally substituted ethyl, R₃₂ may be substituted with R₃₂₁, and R₃₂₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₂₁ comprises a methylene unit, the methylene unit of R₃₂₁ may each independently be unreplaced, or the methylene unit of R₃₂₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₂₁ may be selected from the group consisting of: hydrogen, halogen, —CN, and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₂₁ may be selected from the group consisting of: hydrogen, F, Cl, —CN, and optionally substituted methyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when R₃ may be optionally substituted —SH, R₃ may be substituted with R₃₃, and R₃₃ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₃ comprises a methylene unit, the methylene unit of R₃₃ may each independently be unreplaced, or the methylene unit of R₃₃ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₃ may be selected from the group consisting of: hydrogen and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when R₃₃ may be optionally substituted methyl or optionally substituted ethyl, R₃₃ may be substituted with R₃₃₁, and R₃₃₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₃₁ comprises a methylene unit, the methylene unit of R₃₃₁ may each independently be unreplaced, or the methylene unit of R₃₃₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃₃₁ may be selected from the group consisting of: hydrogen, halogen and —CN.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃ may be selected from the group consisting of: optionally substituted —CH₂Cl, optionally substituted —CH₂SH, optionally substituted —CH₂OH, optionally substituted

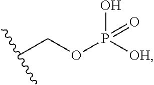 [SMILES: *COP(=O)(O)O]

optionally substituted

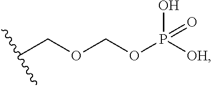 [SMILES: *COCOP(=O)(O)O]

optionally substituted

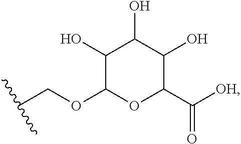 [SMILES: *COC1OC(C(=O)O)C(O)C(O)C1O]

optionally substituted hydroxy, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF3, and optionally substituted —SCH₂CN. For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃ may be selected from the group consisting of: optionally substituted —CH₂Cl, optionally substituted —CH₂SH, optionally substituted —CH₂OH, optionally substituted

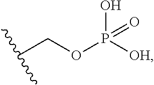 [SMILES: *COP(=O)(O)O]

optionally substituted

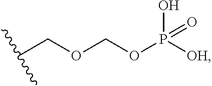 [SMILES: *COCOP(=O)(O)O]

optionally substituted

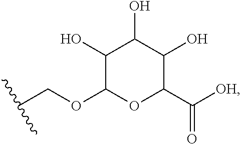 [SMILES: *COC1OC(C(=O)O)C(O)C(O)C1O]

optionally substituted

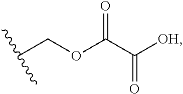 [SMILES: *COC(=O)C(=O)O]

optionally substituted

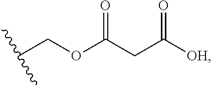 [SMILES: *COC(=O)CC(=O)O]

optionally substituted

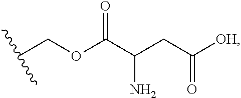 [SMILES: *COC(=O)C(N)CC(=O)O]

optionally substituted

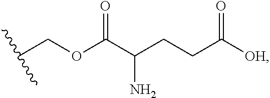 [SMILES: *COC(=O)C(N)CCC(=O)O]

optionally substituted

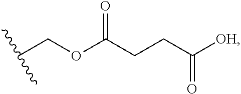 [SMILES: *COC(=O)CCC(=O)O]

optionally substituted

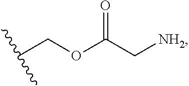 [SMILES: *COC(=O)CN]

optionally substituted

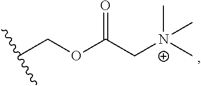 [SMILES: *COC(=O)C[N+](C)(C)C]

optionally substituted

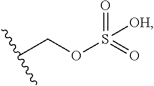 [SMILES: *COS(=O)(=O)O]

optionally substituted

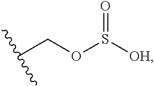 [SMILES: *COS(=O)O]

optionally substituted

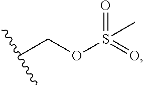 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF3 and optionally substituted —SCH₂CN.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₃ may be selected from —CH₂OH, —SCH₂F,

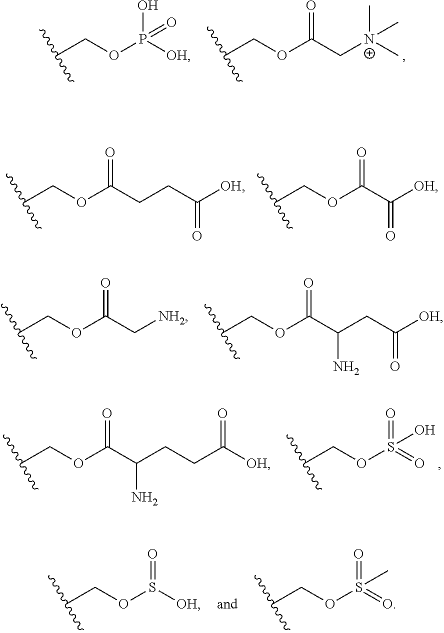

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein Rₚ₁, Rₚ₂ and Rₚ₃ may each independently be selected from the group consisting of hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl, and optionally substituted heteroaryl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when X may be optionally substituted CH, X may be substituted with R₄, and R₄ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₄ comprises a methylene unit, the methylene unit of R₄ may each independently be unreplaced, or the methylene unit of R₄ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₄ may be selected from the group consisting of hydrogen and optionally substituted —OH.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when R₄ may be optionally substituted —OH, R₄ may be substituted with R₄₁, and R₄₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₄₁ comprises a methylene unit, the methylene unit of R₄₁ may each independently be unreplaced, or the methylene unit of R₄₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₄₁ may be selected from the group consisting of: H and optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₄₁ may be optionally substituted methyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein X may be N.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein X may be selected from the group consisting of: CH, C(—O—CH₃), and N.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein W may be absent or W may be selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; wherein, when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein W may be absent.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be selected from the group consisting of —S(═O)—, —S(═O)₂—, —O—, optionally substituted —NH—, optionally substituted alkylene, optionally substituted alkenylene, and optionally substituted alkynylene, wherein, when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when W may be optionally substituted C₁-C₆ alkylene, W may be substituted with R₅, and R₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₅ comprises a methylene unit, the methylene unit of R₅ may each independently be unreplaced, or the methylene unit of R₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₅ may be selected from the group consisting of hydrogen, ═O, optionally substituted C₁-C₆ alkyl, and optionally substituted alcyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₅ may be optionally substituted methyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when R₅ may be optionally substituted methyl, R₅ may be substituted with R₆, and R₆ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₆ may be selected from the group consisting of halogen and optionally substituted alcyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₆ may be F.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₆ may be optionally substituted cyclopropyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₅ may be optionally substituted alcyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₅ may be optionally substituted cyclopropyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when W may be an optionally substituted C₁-C₆ alkylene, and when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein the methylene unit of W may be replaced by a group selected from the group consisting of: —O—, —S—, optionally substituted —NH— and optionally substituted alcylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein W may be methylene, and the methylene unit of W may be replaced by optionally substituted alcylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein W may be methylene, and the methylene unit of W may be replaced by optionally substituted cyclopropyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein W may be methylene, and the methylene unit of W may be replaced by optionally substituted cyclobutyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when W may be optionally substituted —NH—, W may be substituted with R₅, and R₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₅ comprises a methylene unit, the methylene unit of R₅ may each independently be unreplaced, or the methylene unit of R₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
R₅ may be optionally substituted methyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be optionally substituted C₂-C₆ alkenylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be optionally substituted C₂-C₆ alkynylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be absent or W may be optionally substituted —NCH₃—, optionally substituted —CH₂CH₂—, optionally substituted —CH═CH—, —C—C—, optionally substituted —CO—NH—, optionally substituted —OCH₂—, optionally substituted —CH₂O—, optionally substituted —SCH₂—, optionally substituted —CH₂S—, or optionally substituted —NH—CO—.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be absent or W may be selected from the group consisting of the following structures that are optionally substituted:

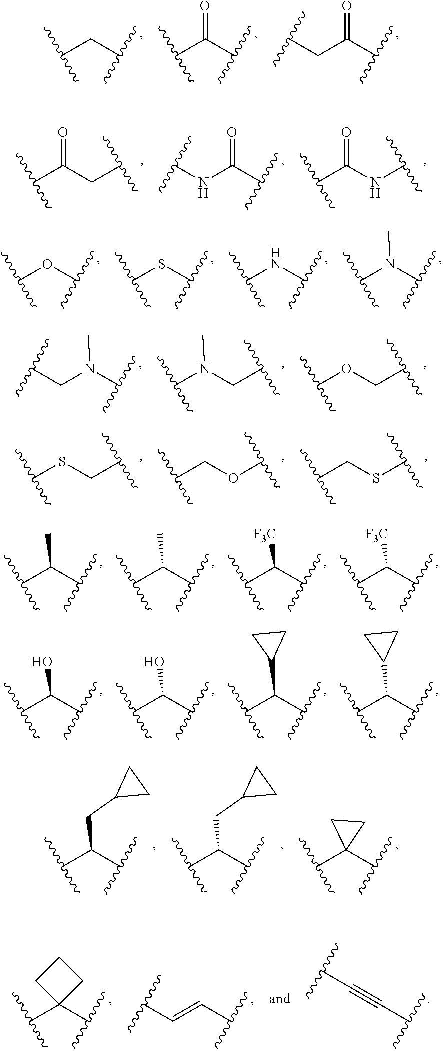

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be absent, or W may be selected from the group consisting of: —S(═O)—, —S(═O)₂—, —O—, optionally substituted —NH—, optionally substituted C₁-C₆ alkylene, optionally substituted C₂-C₆ alkenylene, and optionally substituted C₂-C₆ alkynylene, wherein, when W comprises a methylene unit, the methylene unit of W may each independently be unreplaced, or the methylene unit of W may each independently be replaced by a group selected from the group consisting of: —O—, —S—, and optionally substituted —NH—.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
W may be absent, or W may be selected from the group consisting of: —S(═O)—, —S(═O)₂—, —O—, optionally substituted —NH—, optionally substituted —CH═CH—, —C≡C—, optionally substituted C₁-C₆ alkylene, optionally substituted —OCH₂—, optionally substituted —CH₂O—, optionally substituted —SCH₂—, optionally substituted —CH₂S—, optionally substituted —NHC(═O)—, optionally substituted —COCH₂—, optionally substituted —CH₂NH—, optionally substituted —NHCH(CH₃)—, optionally substituted —NHCH₂—, optionally substituted —CH(CH₃)—, optionally substituted

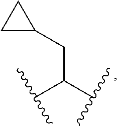 [SMILES: *C(*)CC1CC1]

optionally substituted

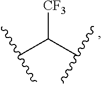 [SMILES: *C(*)C(F)(F)F]

optionally substituted

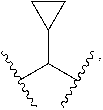 [SMILES: *C(*)C1CC1]

optionally substituted

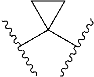 [SMILES: *C1(*)CC1]

optionally substituted

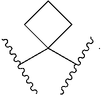 [SMILES: *C1(*)CCC1]

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: hydrogen, protium, deuterium, tritium, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when A comprises a methylene unit, the methylene unit of A may each independently be unreplaced, or the methylene unit of A may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; A may further be substituted with one or more Y, and each Y may be independently selected from the group consisting of hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂. For example, each Y may be independently optionally substituted —CH₂—OH, and Y may be substituted with one or more H or halogen or optionally substituted C₁-C₃ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted C₁-C₆ alkyl, optionally substituted C₃-C₁₀ alcyl, optionally substituted C₂-C₉ aliphatic heterocyclyl, optionally substituted C₆-C₁₀ aryl and optionally substituted C₁-C₉ heteroaryl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when A may be optionally substituted methyl or optionally substituted ethyl, A may be substituted with Rₐ₁, and Rₐ₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₁ comprises a methylene unit, the methylene unit of Rₐ₁ may each independently be unreplaced, or the methylene unit of Rₐ₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
Rₐ₁ may be optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when Rₐ₁ may be optionally substituted methyl, Rₐ₁ may be substituted with Rₐ₁₁, and Rₐ₁₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₁ comprises a methylene unit, the methylene unit of Ra may each independently be unreplaced, or the methylene unit of Rₐ₁₁ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein Rₐ₁₁ may be optionally substituted C₃-C₁₀ alcyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein Rₐ₁ may be optionally substituted cyclopropyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when A may be optionally substituted C₃-C₁₀ alcyl, A may be substituted with Rₐ₂, and Rₐ₂ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₂ comprises a methylene unit, the methylene unit of Rₐ₂ may each independently be unreplaced, or the methylene unit of Rₐ₂ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted cyclopropane, optionally substituted cyclobutane and optionally substituted cyclohexane.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when A may be optionally substituted C₂-C₉ aliphatic heterocyclyl, A may be substituted with Rₐ₃, and Rₐ₃ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₃ comprises a methylene unit, the methylene unit of Rₐ₃ may each independently be unreplaced, or the methylene unit of Rₐ₃ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may comprise one nitrogen heteroatom.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted azetidine and optionally substituted piperidine.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when A may be optionally substituted phenyl, A may be substituted with Rₐ₄, and Rₐ₄ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₄ comprises a methylene unit, the methylene unit of Rₐ₄ may each independently be unreplaced, or the methylene unit of Rₐ₄ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein Rₐ₄ may be selected from the group consisting of: hydrogen, halogen and optionally substituted —OH.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein Rₐ₄ may be selected from the group consisting of hydrogen, C1 and optionally substituted —OH.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when Rₐ₄ may be optionally substituted —OH, Rₐ₄ may be substituted with Rₐ₄₁, and Rₐ₄₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₄₁ comprises a methylene unit, the methylene unit of Rₐ₄₁ may each independently be unreplaced, or the methylene unit of Rₐ₄₁ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
Rₐ₄₁ may be optionally substituted C₁-C₆ alkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
Rₐ₄₁ may be optionally substituted methyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
when A may be optionally substituted C₁-C₉ heteroaryl, A may be substituted with Rₐ₅, and Rₐ₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₅ comprises a methylene unit, the methylene unit of Rₐ₅ may each independently be unreplaced, or the methylene unit of Rₐ₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may comprise one nitrogen heteroatom.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be optionally substituted pyridinyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted C₁-C₆ alkyl, optionally substituted phenyl, and optionally substituted C₃-C₆ alcyl; wherein, when A comprises a methylene unit, the methylene unit of A may each independently be unreplaced, or the methylene unit of A may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; wherein A may be substituted with one or more Y For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted —CH₂—Y, optionally substituted —(CH₂)₂—Y, optionally substituted —(CH₂)₃—Y, optionally substituted —CH₂—CH(—CH₃)—Y, optionally substituted —CH₂(—CH₂-cyclopropyl)-Y, optionally substituted cyclopropyl-Y, optionally substituted cyclobutyl-Y, optionally substituted cyclohexyl-Y, optionally substituted azetidinyl-Y, optionally substituted piperidinyl-Y, optionally substituted-phenyl-Y, optionally substituted-phenyl(—O—CH₃)—Y, optionally substituted-phenyl(—Cl)—Y, and optionally substituted-pyridinyl-Y.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of: optionally substituted —CH₂—Y, optionally substituted —CO—CH₂—Y, optionally substituted —(CH₂)₂—Y, optionally substituted —(CH₂)₃—Y, optionally substituted —CH₂—CH(—CH₃)—Y, optionally substituted —CH₂(—CH₂-cyclopropyl)-Y, optionally substituted

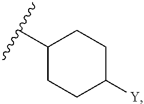

optionally substituted

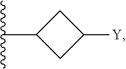

optionally substituted

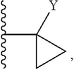

optionally substituted

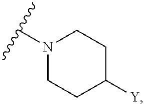

optionally substituted

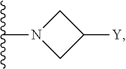

optionally substituted

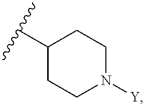

optionally substituted

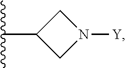

optionally substituted

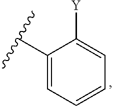

optionally substituted

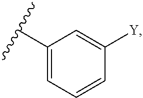

optionally substituted

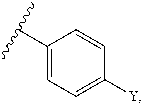

optionally substituted

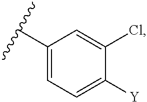

optionally substituted

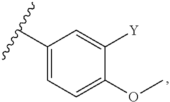

optionally substituted

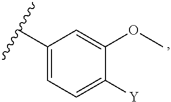

optionally substituted

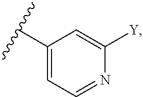

optionally substituted

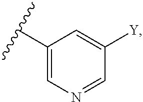

and optionally substituted

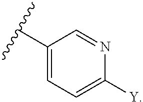

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
A may be selected from the group consisting of optionally substituted

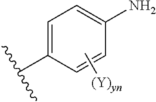

and optionally substituted

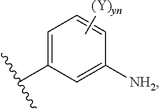

and yn may be a number equal to or greater than 0.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
each Y may be independently selected from the group consisting of: hydrogen, optionally substituted —OH, optionally substituted —CH₂—OH, optionally substituted —SH, and optionally substituted —NH₂.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
each Y may be independently selected from the group consisting of hydrogen, protium, deuterium, tritium, halogen, optionally substituted hydroxy, optionally substituted alkylhydroxy, optionally substituted sulfhydryl, optionally substituted amino, optionally substituted alkyl, optionally substituted alkenyl, optionally substituted alkynyl, optionally substituted cycloalkyl, optionally substituted heterocycloalkyl, optionally substituted aryl, and optionally substituted heteroaryl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein
each Y may be independently selected from the group consisting of: optionally substituted hydroxy, optionally substituted alkylhydroxy, optionally substituted sulfhydryl, optionally substituted amino and optionally substituted C₁-C₆ alkyl. For example, Y may be optionally substituted —(CH₂)ym-OH, ym may be a number equal to or greater than 0, and the methylene units of Y may each independently be replaced by any group. For example, Y may be optionally substituted C₁-C₆ alkylhydroxy, and Y may be substituted with H, halogen, optionally substituted C₁-C₆ alkyl, or optionally substituted C₁-C₆ haloalkyl. For example, Y may be optionally substituted C₁-C₃ alkylhydroxy, and Y may be substituted with H, halogen, optionally substituted C₁-C₃ alkyl, or optionally substituted C₁-C₃ haloalkyl.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when Y may be hydrogen, A comprises optionally substituted aliphatic heterocyclylene, and the aliphatic heterocyclylene of A comprises the ring-forming atom Xa, wherein Xa may be optionally substituted NH.

For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when Y may be hydrogen, A may be selected from the group consisting of: optionally substituted azetidinyl-Y and optionally substituted piperidinyl-Y For example, provided is a compound comprising a structure shown as formula (I), formula (I-a1) or formula (I-a2), or a tautomer, a mesomer, a racemate, an enantiomer or a diastereoisomer thereof, or a mixture thereof, or a pharmaceutically acceptable salt thereof, wherein when Y may be hydrogen, A may be selected from the group consisting of: optionally substituted

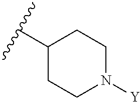

and optionally substituted

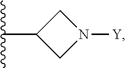

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1) or formula (I-a2):

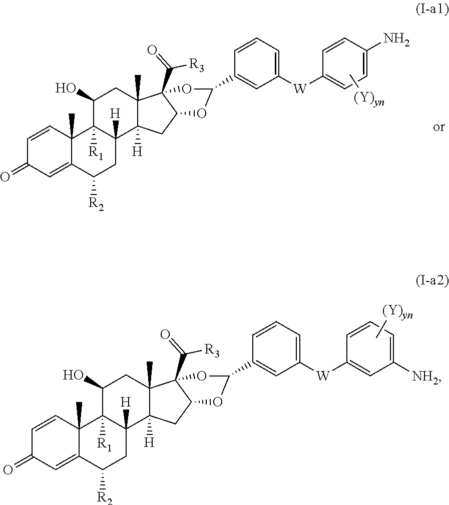

wherein R₁ may be selected from the group consisting of hydrogen, halogen, and optionally substituted methyl, R₂ may be selected from the group consisting of: hydrogen, halogen, and optionally substituted methyl, R₃ may be selected from the group consisting of optionally substituted —CH₂Cl, optionally substituted —CH₂SH, optionally substituted —CH₂OH, optionally substituted

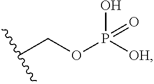 [SMILES: *COP(=O)(O)O]

optionally substituted

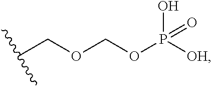 [SMILES: *COCOP(=O)(O)O]

optionally substituted

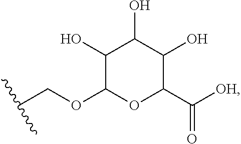 [SMILES: *COC1OC(C(=O)O)C(O)C(O)C1O]

optionally substituted

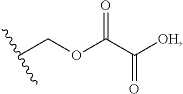 [SMILES: *COC(=O)C(=O)O]

optionally substituted

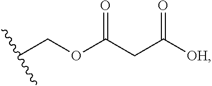 [SMILES: *COC(=O)CC(=O)O]

optionally substituted

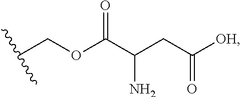 [SMILES: *COC(=O)C(N)CC(=O)O]

optionally substituted

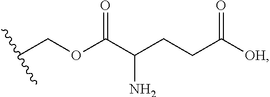 [SMILES: *COC(=O)C(N)CCC(=O)O]

optionally substituted

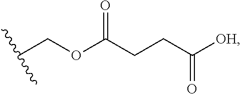 [SMILES: *COC(=O)CCC(=O)O]

optionally substituted

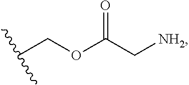 [SMILES: *COC(=O)CN]

optionally substituted

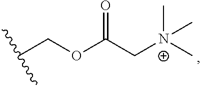 [SMILES: *COC(=O)C[N+](C)(C)C]

optionally substituted

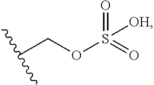 [SMILES: *COS(=O)(=O)O]

optionally substituted

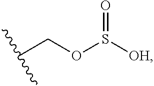 [SMILES: *COS(=O)O]

optionally substituted

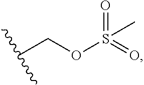 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1) or formula (I-a2):

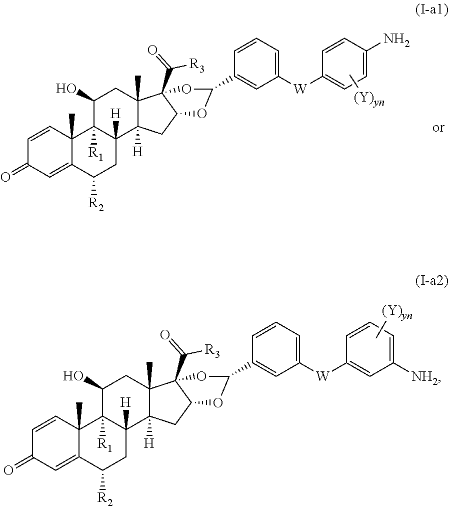

wherein
R₁ may be selected from the group consisting of: hydrogen and halogen, R₂ may be selected from the group consisting of: hydrogen and halogen, R₃ may be selected from the group consisting of: —CH₂OH, —SCH₂F,

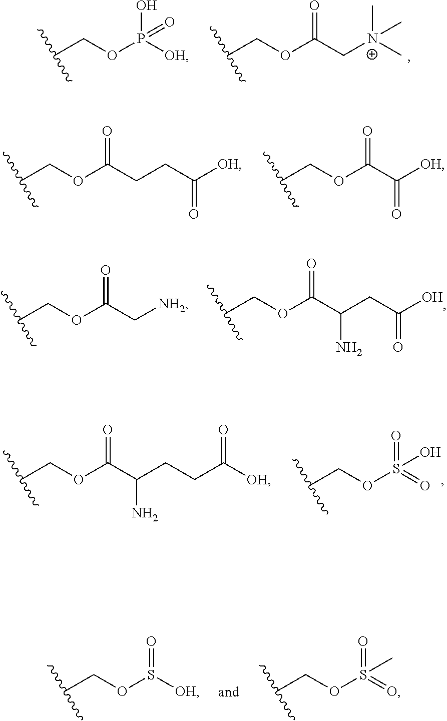

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof,
wherein the compound comprises a structure that may be selected from the group consisting of:

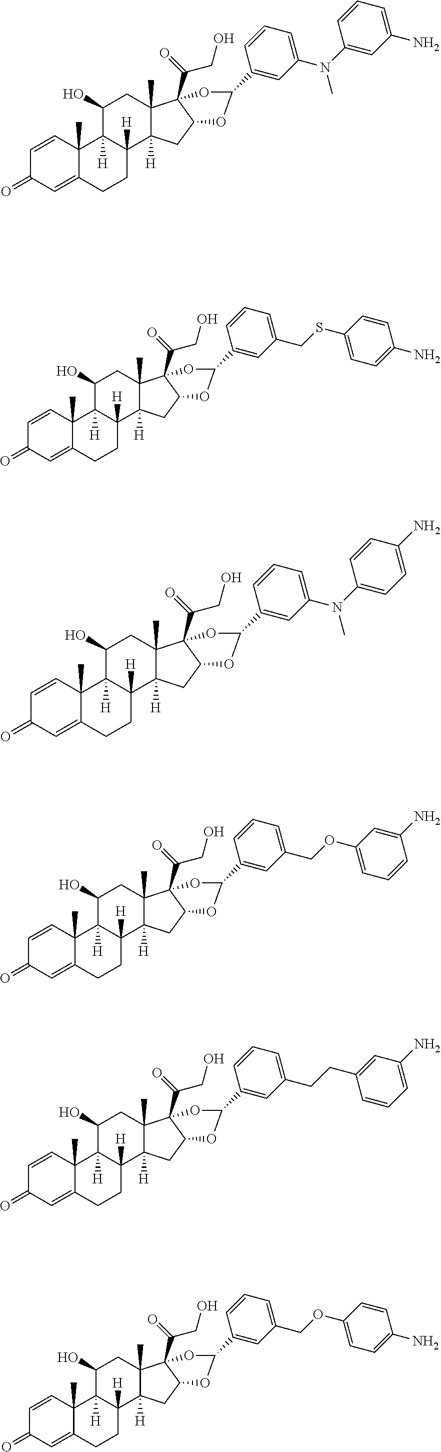

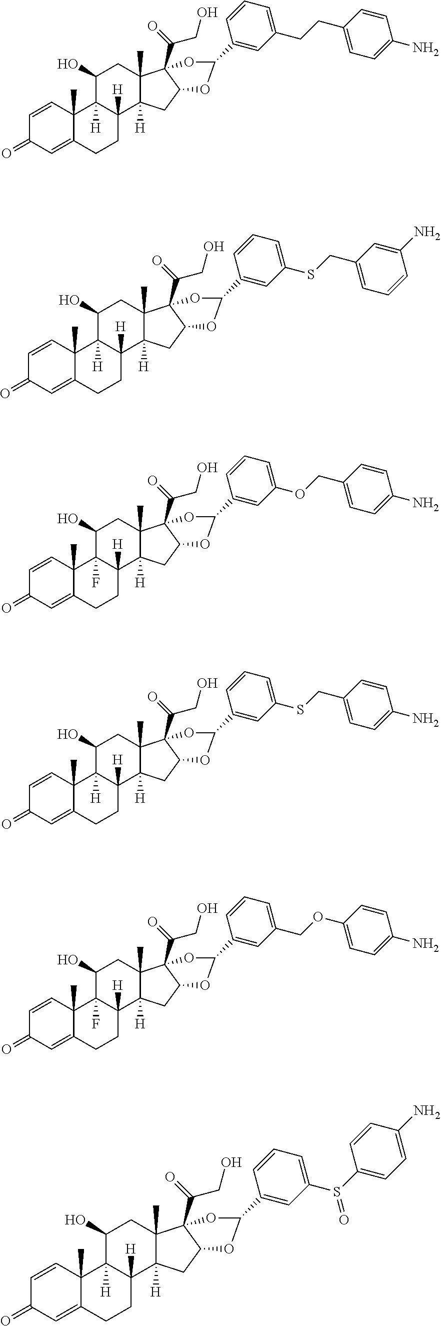

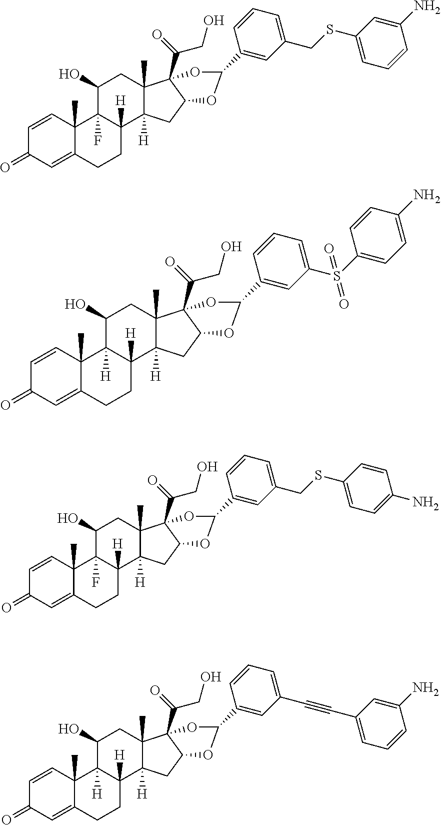

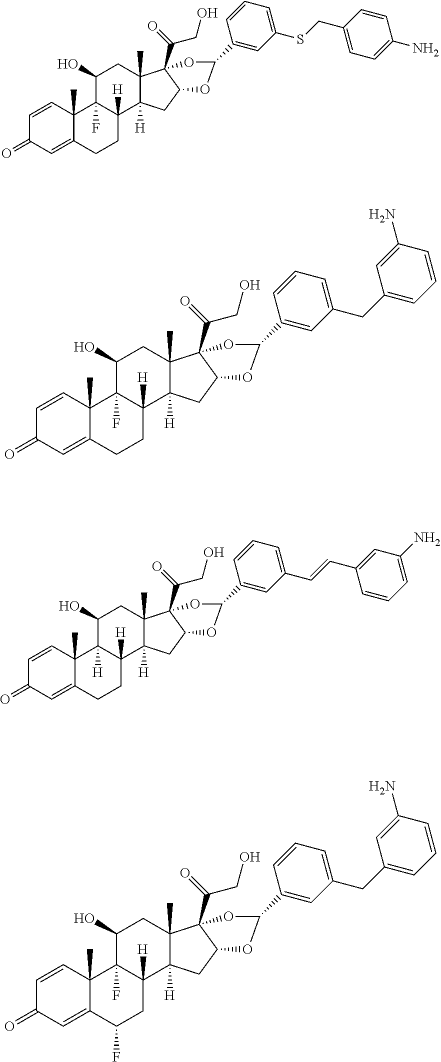

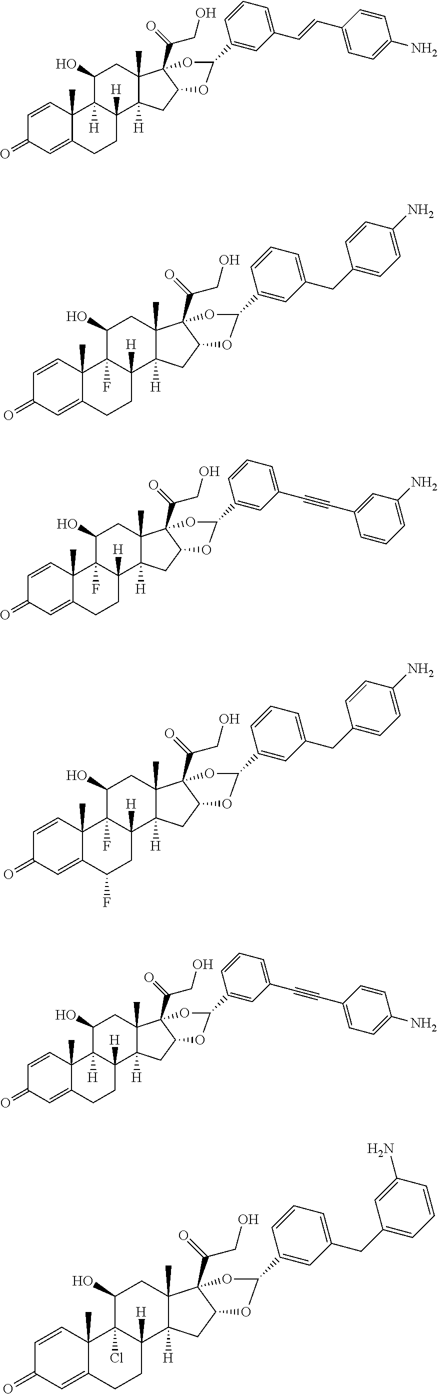

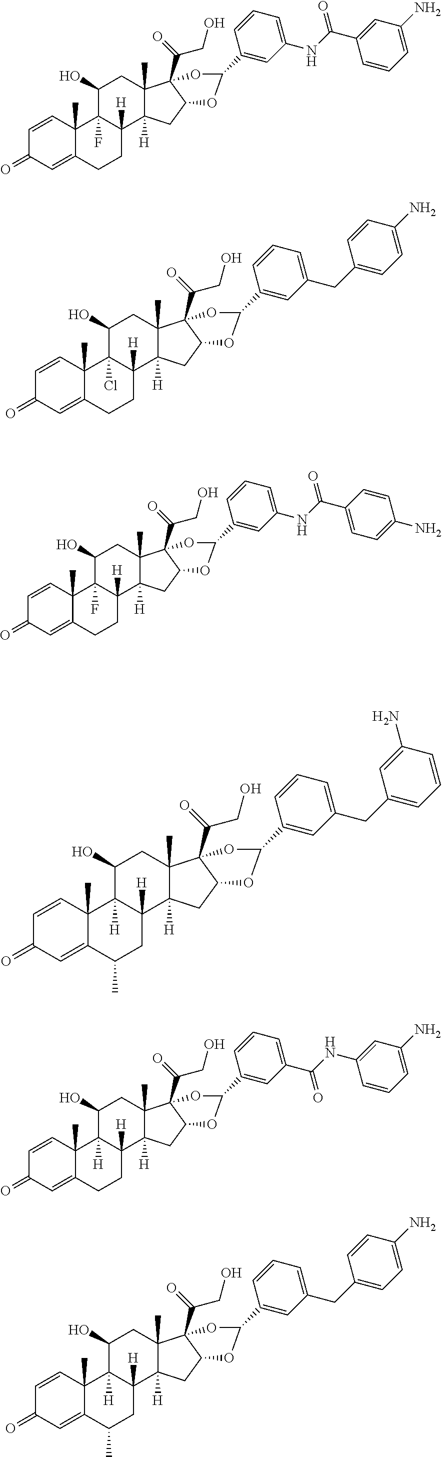

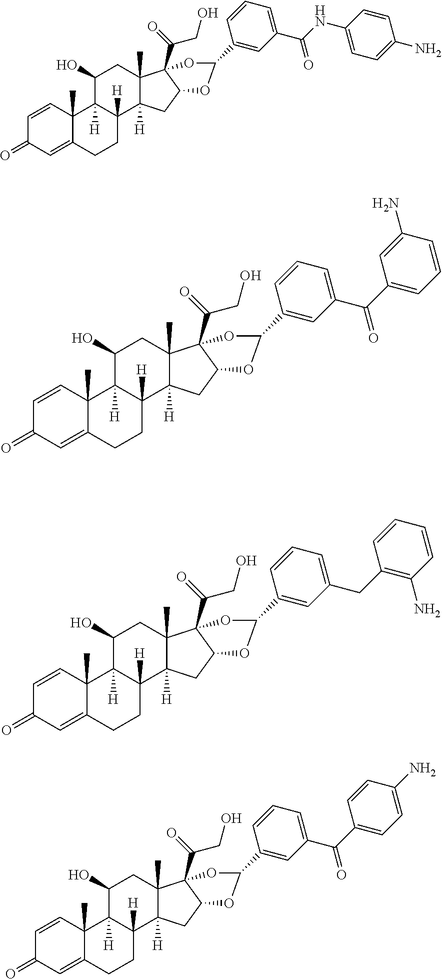

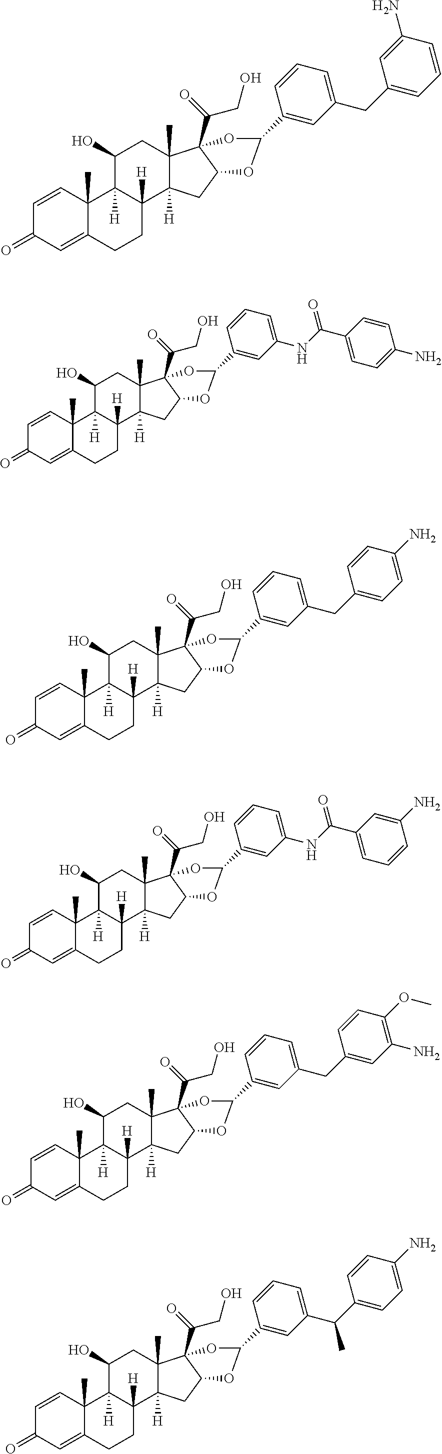

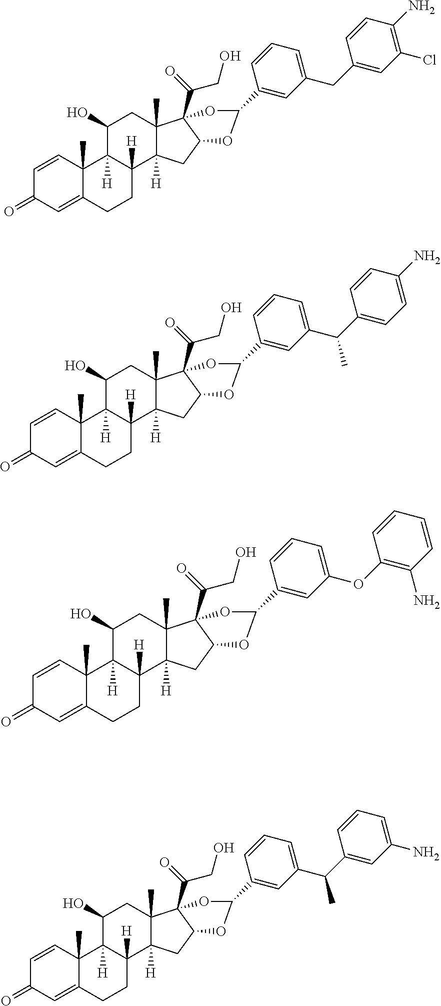

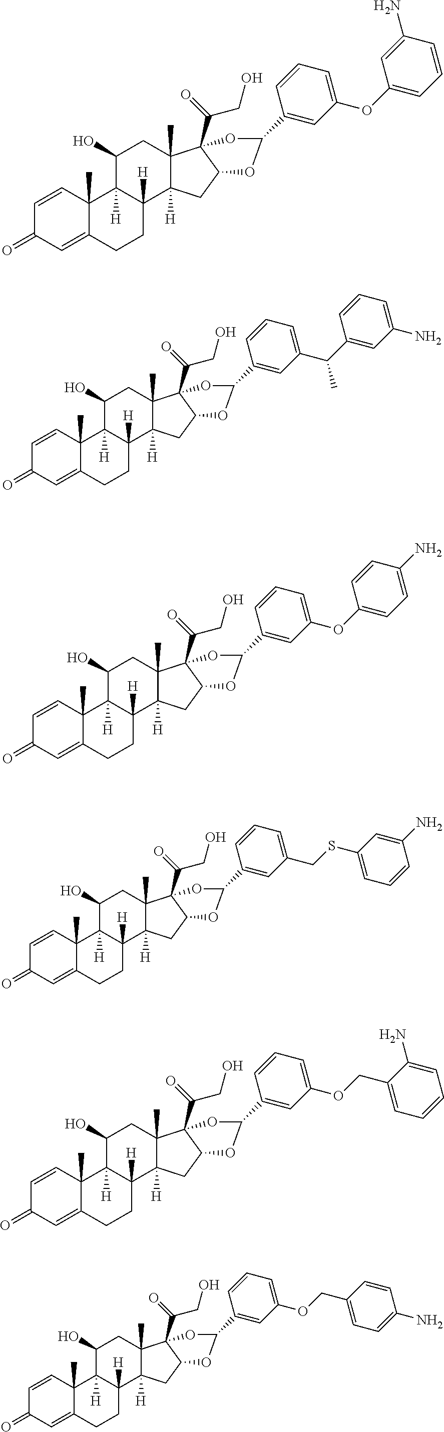

The present application provides a compound or a tautomer, a mesomer, a racemate, an enantiomer or a diastereoisomer thereof: or a mixture thereof: or a pharmaceutically acceptable salt thereof, wherein the compound comprises a structure shown as formula (II):

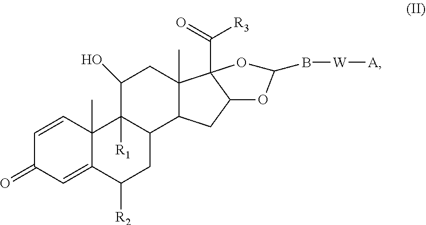

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and each Y may be independently selected from the group consisting of: hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂. The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1) or formula (II-a2):

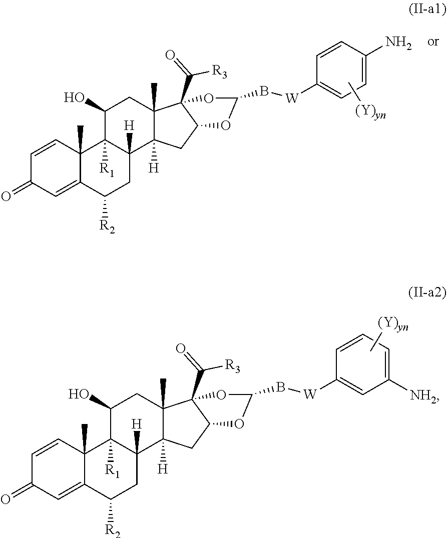

wherein
R₁, R₂, R₃ and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1) or formula (II-b2):

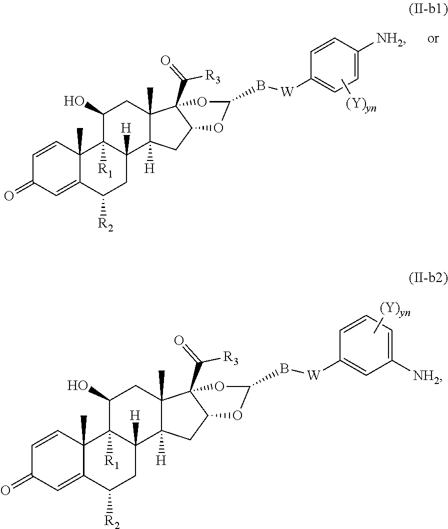

wherein
R₁, R₂, R₃ and Y may each independently be any group;
B may be selected from the group consisting of: optionally substituted

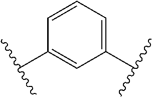 [SMILES: *c1cccc(*)c1]

and optionally substituted heteroarylene; for example, B may be selected from the group consisting of: optionally substituted

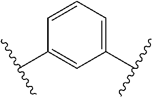 [SMILES: *c1cccc(*)c1]

and optionally substituted C₂-C₄ heteroarylene; W may be absent or W may be any group, wherein, when B may be optionally substituted

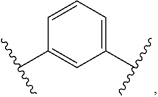 [SMILES: *c1cccc(*)c1]

W may not be —S—; yn may be a number equal to or greater than 0.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₁, R₂ and R₃ may each independently be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₁, R₂, and R₃ comprise methylene units, the methylene units of R₁, R₂, and R₃ may each independently be unreplaced, or the methylene units of R₁, R₂, and R₃ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₁ may be selected from the group consisting of: hydrogen, protium, deuterium, tritium, halogen, and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₁ may be selected from the group consisting of: hydrogen, F, Cl, Br, and optionally substituted methyl. For example, R₁ may be H or fluorine.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₂ may be selected from the group consisting of: hydrogen, protium, deuterium, tritium, halogen, and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₂ may be selected from the group consisting of: hydrogen, F, Cl, Br, and optionally substituted methyl. For example, R₂ may be H or fluorine.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃ may be selected from the group consisting of hydrogen, optionally substituted —OH, optionally substituted —SH, and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃ may be optionally substituted methyl, R₃ may be substituted with R₃₁, and R₃₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₁ comprises a methylene unit, the methylene unit of R₃₁ may each independently be unreplaced, or the methylene unit of R₃₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₁ may be selected from the group consisting of: hydrogen, halogen, optionally substituted —OH, and optionally substituted —SH.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃₁ may be optionally substituted —OH, R₃₁ may be substituted with R₃₁₁, and R₃₁₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₁₁ comprises a methylene unit, the methylene unit of R₃₁₁ may each independently be unreplaced, or the methylene unit of R₃₁₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₁₁ may be selected from the group consisting of: hydrogen, optionally substituted —P(═O)H₂, optionally substituted C₁-C₆ alkyl, and optionally substituted C₂-C₉ aliphatic heterocyclylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₁₁ may be selected from the group consisting of: hydrogen, optionally substituted

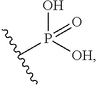 [SMILES: *P(=O)(O)O]

optionally substituted

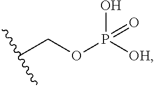 [SMILES: *COP(=O)(O)O]

and optionally substituted

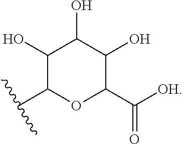 [SMILES: *C1OC(C(=O)O)C(O)C(O)C1O]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃ may be optionally substituted —OH, R₃ may be substituted with R₃₂, and R₃₂ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₂ comprises a methylene unit, the methylene unit of R₃₂ may each independently be unreplaced, or the methylene unit of R₃₂ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₂ may be selected from the group consisting of hydrogen, and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃₂ may be optionally substituted methyl or optionally substituted ethyl, R₃₂ may be substituted with R₃₂₁, and R₃₂₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₂₁ comprises a methylene unit, the methylene unit of R₃₂₁ may each independently be unreplaced, or the methylene unit of R₃₂₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₂₁ may be selected from the group consisting of: hydrogen, halogen, —CN, and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₂₁ may be selected from the group consisting of: hydrogen, F, Cl, —CN, and optionally substituted methyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃ may be optionally substituted —SH, R₃ may be substituted with R₃₃, and R₃₃ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₃ comprises a methylene unit, the methylene unit of R₃₃ may each independently be unreplaced, or the methylene unit of R₃₃ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₃ may be selected from the group consisting of: hydrogen and optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₃₃ may be optionally substituted methyl or optionally substituted ethyl, R₃₃ may be substituted with R₃₃₁, and R₃₃₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₃₃₁ comprises a methylene unit, the methylene unit of R₃₃₁ may each independently be unreplaced, or the methylene unit of R₃₃₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃₃₁ may be selected from the group consisting of: hydrogen, halogen and —CN.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃ may be selected from the group consisting of: optionally substituted —CH₂Cl, optionally substituted —CH₂SH, optionally substituted —CH₂OH, optionally substituted

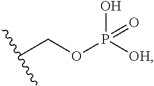 [SMILES: *COP(=O)(O)O]

optionally substituted

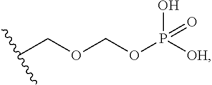 [SMILES: *COCOP(=O)(O)O]

optionally substituted

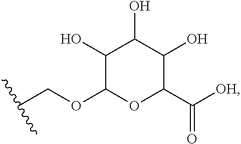 [SMILES: *COC1OC(C(=O)O)C(O)C(O)C1O]

optionally substituted hydroxy, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃ may be selected from the group consisting of: optionally substituted —CH₂Cl, optionally substituted —CH₂SH, optionally substituted —CH₂OH, optionally substituted

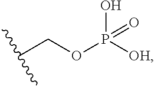 [SMILES: *COP(=O)(O)O]

optionally substituted

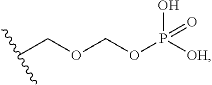 [SMILES: *COCOP(=O)(O)O]

optionally substituted

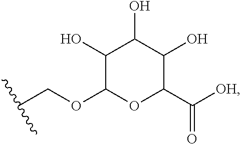 [SMILES: *COC1OC(C(=O)O)C(O)C(O)C1O]

optionally substituted

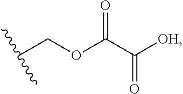 [SMILES: *COC(=O)C(=O)O]

optionally substituted

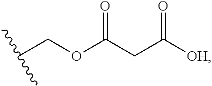 [SMILES: *COC(=O)CC(=O)O]

optionally substituted

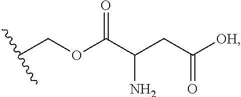 [SMILES: *COC(=O)C(N)CC(=O)O]

optionally substituted

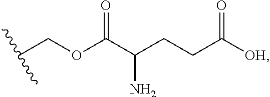 [SMILES: *COC(=O)C(N)CCC(=O)O]

optionally substituted

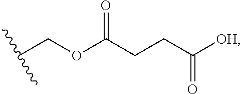 [SMILES: *COC(=O)CCC(=O)O]

optionally substituted

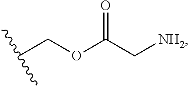 [SMILES: *COC(=O)CN]

optionally substituted

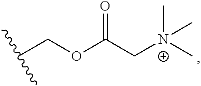 [SMILES: *COC(=O)C[N+](C)(C)C]

optionally substituted

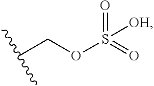 [SMILES: *COS(=O)(=O)O]

optionally substituted

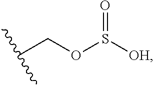 [SMILES: *COS(=O)O]

optionally substituted

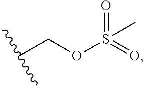 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃ and optionally substituted —SCH₂CN.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₃ may be selected from —CH₂OH, —SCH₂F,

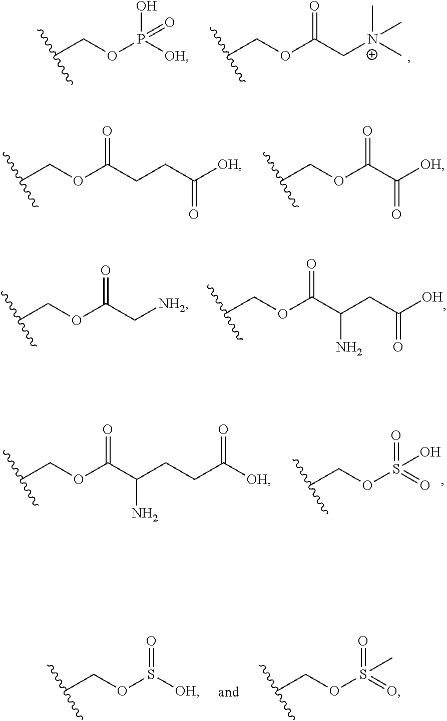

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted C₃-C₁₀ alcylene, optionally substituted C₂-C₉ aliphatic heterocyclylene and optionally substituted heteroarylene. For example, B may be selected from the group consisting of: optionally substituted C₃-C₁₀ alcylene, optionally substituted C₂-C₉ aliphatic heterocyclylene and optionally substituted C₂-C₄ heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may comprise one nitrogen heteroatom, one sulfur heteroatom and/or one oxygen heteroatom. For example, B may comprise one nitrogen heteroatom and/or one sulfur heteroatom.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted pyridinyl, optionally substituted pyrrolyl and optionally substituted furanyl. For example, B may be selected from the group consisting of: optionally substituted pyrrolyl and optionally substituted thienyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be optionally substituted methylpyrrolyl. For example, B may be furanyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted

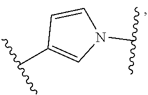 [SMILES: *c1ccn(*)c1]

optionally substituted

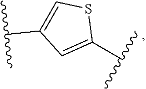 [SMILES: *c1csc(*)c1]

optionally substituted

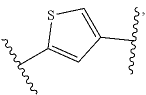 [SMILES: *c1csc(*)c1]

and optionally substituted

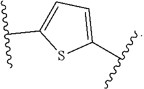 [SMILES: *c1ccc(*)s1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of optionally substituted

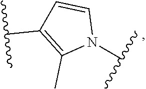 [SMILES: *c1ccn(*)c1C]

optionally substituted

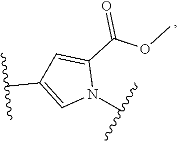 [SMILES: *c1cc(C(=O)OC)n(*)c1]

optionally substituted

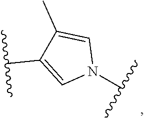 [SMILES: *c1cn(*)cc1C]

optionally substituted

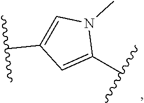 [SMILES: *c1cc(*)n(C)c1]

optionally substituted

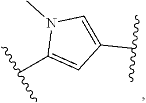 [SMILES: *c1cc(*)n(C)c1]

optionally substituted

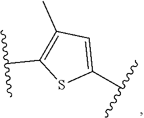 [SMILES: *c1cc(C)c(*)s1]

optionally substituted

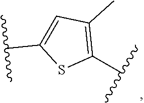 [SMILES: *c1cc(C)c(*)s1]

optionally substituted

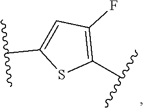 [SMILES: *c1cc(F)c(*)s1]

optionally substituted

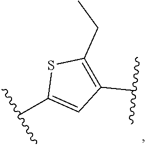 [SMILES: *c1cc(*)c(CC)s1]

optionally substituted

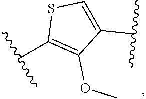 [SMILES: *c1csc(*)c1OC]

optionally substituted

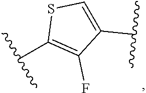 [SMILES: *c1csc(*)c1F]

optionally substituted

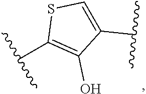 [SMILES: *c1csc(*)c1O]

optionally substituted

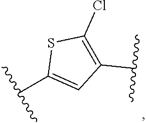 [SMILES: *c1cc(*)c(Cl)s1]

optionally substituted

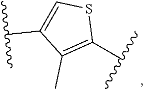 [SMILES: *c1csc(*)c1C]

and optionally substituted

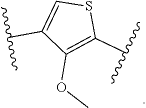 [SMILES: *c1csc(*)c1OC]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted

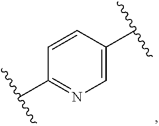 [SMILES: *c1ccc(*)nc1]

optionally substituted

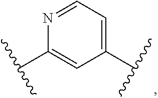 [SMILES: *c1ccnc(*)c1]

optionally substituted

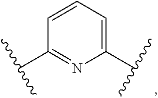 [SMILES: *c1cccc(*)n1]

optionally substituted

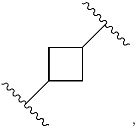 [SMILES: *C1CC(*)C1]

optionally substituted

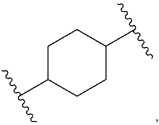 [SMILES: *C1CCC(*)CC1]

optionally substituted

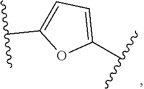 [SMILES: *c1ccc(*)o1]

optionally substituted

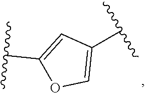 [SMILES: *c1coc(*)c1]

optionally substituted

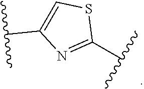 [SMILES: *c1csc(*)n1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted

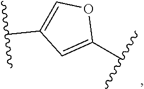 [SMILES: *c1coc(*)c1]

and optionally substituted

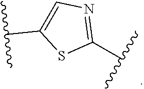 [SMILES: *c1cnc(*)s1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted cyclobutylene and optionally substituted cyclohexylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of optionally substituted

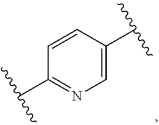 [SMILES: *c1ccc(*)nc1]

optionally substituted

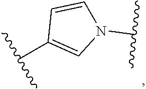 [SMILES: *c1ccn(*)c1]

optionally substituted

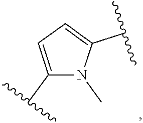 [SMILES: *c1ccc(*)n1C]

optionally substituted

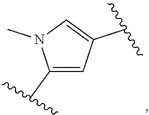 [SMILES: *c1cc(*)n(C)c1]

optionally substituted

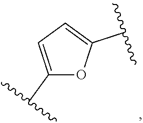 [SMILES: *c1ccc(*)o1]

optionally substituted

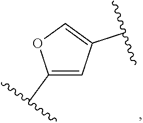 [SMILES: *c1coc(*)c1]

optionally substituted

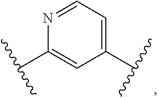 [SMILES: *c1ccnc(*)c1]

optionally substituted

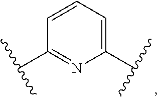 [SMILES: *c1cccc(*)n1]

optionally substituted

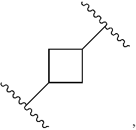 [SMILES: *C1CC(*)C1]

and optionally substituted

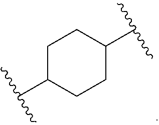 [SMILES: *C1CCC(*)CC1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted C₂-C₄ heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted

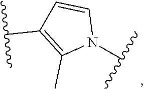 [SMILES: *c1ccn(*)c1C]

optionally substituted

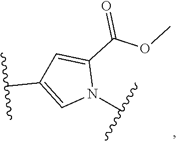 [SMILES: *c1cc(C(=O)OC)n(*)c1]

optionally substituted

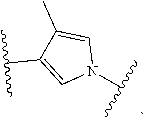 [SMILES: *c1cn(*)cc1C]

optionally substituted

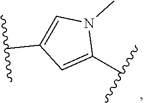 [SMILES: *c1cc(*)n(C)c1]

optionally substituted

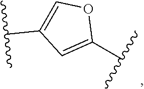 [SMILES: *c1coc(*)c1]

optionally substituted

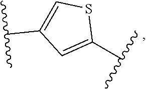 [SMILES: *c1csc(*)c1]

optionally substituted

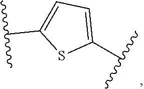 [SMILES: *c1ccc(*)s1]

optionally substituted

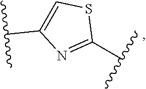 [SMILES: *c1csc(*)n1]

and optionally substituted

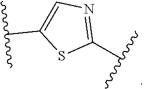 [SMILES: *c1cnc(*)s1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be selected from the group consisting of: optionally substituted

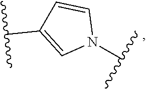 [SMILES: *c1ccn(*)c1]

optionally substituted

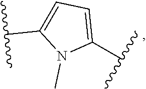 [SMILES: *c1ccc(*)n1C]

optionally substituted

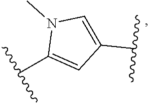 [SMILES: *c1cc(*)n(C)c1]

optionally substituted

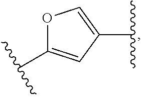 [SMILES: *c1coc(*)c1]

and optionally substituted

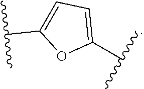 [SMILES: *c1ccc(*)o1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
B may be substituted with R_{B}, and R_{B} may be any group. For example, R_{B} may be selected from the group consisting of: hydrogen, halogen, optionally substituted hydroxy, optionally substituted C₁-C₆ alkyl, and optionally substituted C₁-C₆ ester groups. For example, R_{B} may be selected from the group consisting of hydrogen, halogen, optionally substituted hydroxy, optionally substituted C₁-C₃ alkyl, and optionally substituted C₁-C₃ ester groups. For example, R_{B} may be hydrogen, fluorine, chlorine, optionally substituted C₁-C₃ alkyl, optionally substituted hydroxy, optionally substituted hydroxy-C₁-C₃ alkyl, or optionally substituted

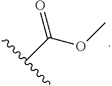 [SMILES: *C(=O)OC]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent or W may be selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; wherein, when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be selected from the group consisting of —S(═O)—, —S(═O)₂—, —O—, —S—, optionally substituted —NH—, optionally substituted alkylene, optionally substituted alkenylene, and optionally substituted alkynylene, wherein, when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when W may be optionally substituted C₁-C₆ alkylene, W may be substituted with R₅, and R₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₅ comprises a methylene unit, the methylene unit of R₅ may each independently be unreplaced, or the methylene unit of R₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₅ may be selected from the group consisting of hydrogen, ═O, optionally substituted C₁-C₆ alkyl, and optionally substituted alcyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₅ may be optionally substituted methyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when R₅ may be optionally substituted methyl, R₅ may be substituted with R₆, and R₆ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₆ may be selected from the group consisting of: halogen and optionally substituted alcyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₆ may be F.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₆ may be optionally substituted cyclopropyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₅ may be optionally substituted alcyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₅ may be optionally substituted cyclopropyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when W may be an optionally substituted C₁-C₆ alkylene, and when W comprises a methylene unit, the methylene unit of W may each independently be unreplaced, or the methylene unit of W may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein the methylene unit of W may be replaced by a group selected from the group consisting of: —O—, —S—, and optionally substituted —NH—.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent, or W may be selected from the group consisting of: —S(═O)—, —S(═O)₂—, —O—, optionally substituted —NH—, optionally substituted C₁-C₆ alkylene, optionally substituted C₂-C6 alkenylene, and optionally substituted C₂-C₆ alkynylene, wherein, when W comprises a methylene unit, the methylene unit of W may each independently be unreplaced, or the methylene unit of W may each independently be replaced by a group selected from the group consisting of: —O—, —S—, optionally substituted —NH—, and optionally substituted alcylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be methylene, and the methylene unit of W may be replaced by optionally substituted alcylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be methylene, and the methylene unit of W may be replaced by optionally substituted cyclopropyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be methylene, and the methylene unit of W may be replaced by optionally substituted cyclobutyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent, or W may be selected from the group consisting of: —S(═O)—, —S(═O)₂—, —O—, —S—, optionally substituted —NH—, optionally substituted —CH═CH—, —C≡C—, optionally substituted C₁-C₆ alkylene, optionally substituted —OCH₂—, optionally substituted —CH₂O—, optionally substituted —SCH₂—, optionally substituted —CH₂S—, —CO—, optionally substituted —NHC(═O)—, optionally substituted —COCH₂—, optionally substituted —CH₂NH—, optionally substituted —NHCH(CH₃)—, optionally substituted —NHCH₂—, optionally substituted —CH(CH₃)—, optionally substituted

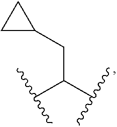 [SMILES: *C(*)CC1CC1]

optionally substituted

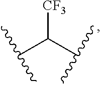 [SMILES: *C(*)C(F)(F)F]

optionally substituted

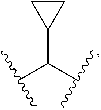 [SMILES: *C(*)C1CC1]

optionally substituted

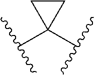 [SMILES: *C1(*)CC1]

and optionally substituted

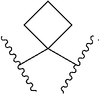 [SMILES: *C1(*)CCC1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when W may be optionally substituted —NH—, W may be substituted with R₅, and R₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when R₅ comprises a methylene unit, the methylene unit of R₅ may each independently be unreplaced, or the methylene unit of R₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
R₅ may be optionally substituted methyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be optionally substituted C₂-C₆ alkenylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be optionally substituted C₂-C₆ alkynylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be absent or W may be selected from the group consisting of the following structures that are optionally substituted:

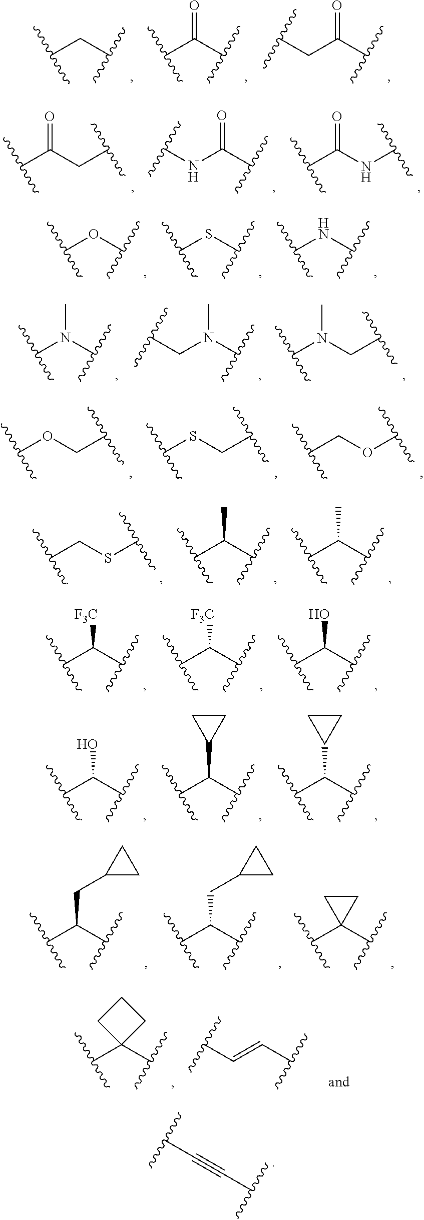

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
W may be selected from the group consisting of the following structures that are optionally substituted: optionally substituted —CH₂—, optionally substituted —CH(CH₃)—, and optionally substituted

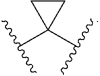 [SMILES: *C1(*)CC1]

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: hydrogen, protium, deuterium, tritium, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when A comprises a methylene unit, the methylene unit of A may each independently be unreplaced, or the methylene unit of A may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; A may further be substituted with one or more Y, and each Y may be independently selected from the group consisting of hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂. For example, each Y may be independently optionally substituted —CH₂—OH, and Y may be substituted with one or more H or halogen or optionally substituted C₁-C₃ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted C₁-C₆ alkyl, optionally substituted C₃-C₁₀ alcyl, optionally substituted C₂-C₉ aliphatic heterocyclyl, optionally substituted C₆-C₁₀ aryl and optionally substituted C₁-C₉ heteroaryl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when A may be optionally substituted methyl or optionally substituted ethyl, A may be substituted with Rai, and Rₐ₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₁ comprises a methylene unit, the methylene unit of Rₐ₁ may each independently be unreplaced, or the methylene unit of Rₐ₁ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₁ may be optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when Rₐ₁ may be optionally substituted methyl, Rₐ₁ may be substituted with Rₐ₁₁, and Rₐ₁₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₁₁ comprises a methylene unit, the methylene unit of Rₐ₁₁ may each independently be unreplaced, or the methylene unit of Rₐ₁₁ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₁₁ may be optionally substituted C₃-C₁₀ alcyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₁₁ may be optionally substituted cyclopropyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when A may be optionally substituted C₃-C₆ alcyl, A may be substituted with Rₐ₂, and Rₐ₂ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₂ comprises a methylene unit, the methylene unit of Rₐ₂ may each independently be unreplaced, or the methylene unit of Rₐ₂ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted cyclopropane, optionally substituted cyclobutane and optionally substituted cyclohexane.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when A may be optionally substituted C₃-C₅ aliphatic heterocyclyl, A may be substituted with Rₐ₃, and Rₐ₃ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₃ comprises a methylene unit, the methylene unit of Rₐ₃ may each independently be unreplaced, or the methylene unit of Rₐ₃ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may comprise one nitrogen heteroatom.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted azetidine and optionally substituted piperidine.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when A may be optionally substituted phenyl, A may be substituted with Rₐ₄, and Rₐ₄ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₄ comprises a methylene unit, the methylene unit of Rₐ₄ may each independently be unreplaced, or the methylene unit of Rₐ₄ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₄ may be selected from the group consisting of: hydrogen, halogen and optionally substituted —OH.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₄ may be selected from the group consisting of hydrogen, C1 and optionally substituted —OH.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when Rₐ₄ may be optionally substituted —OH, Rₐ₄ may be substituted with Rₐ₄₁, and Rₐ₄₁ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₄₁ comprises a methylene unit, the methylene unit of Rₐ₄₁ may each independently be unreplaced, or the methylene unit of Rₐ₄₁ may each independently be replaced by a group selected from the group consisting of —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₄₁ may be optionally substituted C₁-C₆ alkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
Rₐ₄₁ may be optionally substituted methyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when A may be optionally substituted C5 heteroaryl, A may be substituted with Rₐ₅, and Rₐ₅ may be selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₅ comprises a methylene unit, the methylene unit of Rₐ₅ may each independently be unreplaced, or the methylene unit of Rₐ₅ may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may comprise one nitrogen heteroatom.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be optionally substituted pyridinyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted C₁-C₆ alkyl, optionally substituted phenyl, and optionally substituted C₃-C₆ alcyl; wherein, when A comprises a methylene unit, the methylene unit of A may each independently be unreplaced, or the methylene unit of A may each independently be replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene; wherein A may be substituted with one or more Y.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted —CH₂—Y, optionally substituted —(CH₂)₂—Y, optionally substituted —(CH₂)₃—Y, optionally substituted —CH₂—CH(—CH₃)—Y, optionally substituted —CH₂(—CH₂-cyclopropyl)-Y, optionally substituted cyclopropyl-Y, optionally substituted cyclobutyl-Y, optionally substituted cyclohexyl-Y, optionally substituted azetidinyl-Y, optionally substituted piperidinyl-Y, optionally substituted-phenyl-Y, optionally substituted-phenyl(—O—CH₃)—Y, optionally substituted-phenyl(—Cl)—Y, and optionally substituted-pyridinyl-Y.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of: optionally substituted —CH₂—Y, optionally substituted —(CH₂)₂—Y, optionally substituted —(CH₂)₃—Y, optionally substituted —CH₂—CH(—CH₃)—Y, optionally substituted —CH₂(—CH₂-cyclopropyl)-Y, optionally substituted

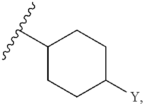

optionally substituted

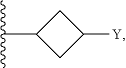

optionally substituted

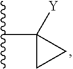

optionally substituted

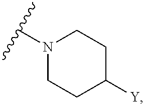

optionally substituted

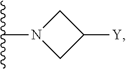

optionally substituted

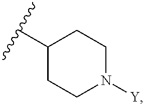

optionally substituted

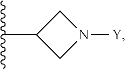

optionally substituted

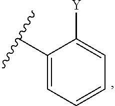

optionally substituted

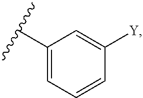

optionally substituted

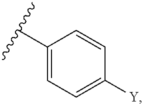

optionally substituted

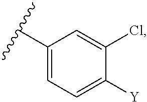

optionally substituted

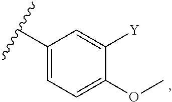

optionally substituted

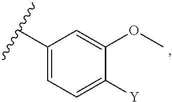

optionally substituted

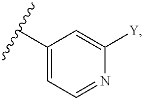

optionally substituted

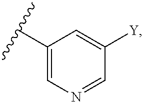

and optionally substituted

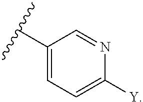

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
A may be selected from the group consisting of optionally substituted

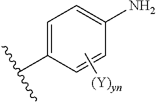

and optionally substituted

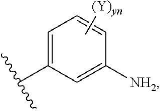

and yn may be a number equal to or greater than 0.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
each Y may be independently selected from the group consisting of: hydrogen, optionally substituted —OH, optionally substituted —CH₂—OH, optionally substituted —SH, and optionally substituted —NH₂.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
each Y may be independently selected from the group consisting of hydrogen, protium, deuterium, tritium, halogen, optionally substituted hydroxy, optionally substituted alkylhydroxy, optionally substituted sulfhydryl, optionally substituted amino, optionally substituted alkyl, optionally substituted alkenyl, optionally substituted alkynyl, optionally substituted cycloalkyl, optionally substituted heterocycloalkyl, optionally substituted aryl, and optionally substituted heteroaryl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
each Y may be independently selected from the group consisting of: optionally substituted hydroxy, optionally substituted alkylhydroxy, optionally substituted sulfhydryl, optionally substituted amino and optionally substituted C₁-C₆ alkyl. For example, Y may be optionally substituted —(CH₂)ym-OH, ym may be a number equal to or greater than 0, and the methylene units ofY may each independently be replaced by any group. For example, Y may be optionally substituted C₁-C₆ alkylhydroxy, and Y may be substituted with H, halogen, optionally substituted C₁-C₆ alkyl, or optionally substituted C₁-C₆ haloalkyl. For example, Y may be optionally substituted C₁-C₃ alkylhydroxy, and Y may be substituted with H, halogen, optionally substituted C₁-C₃ alkyl, or optionally substituted C₁-C₃ haloalkyl.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when Y may be hydrogen, A comprises optionally substituted aliphatic heterocyclylene, and the aliphatic heterocyclylene of A comprises the ring-forming atom Xa, wherein Xa may be optionally substituted NH.

For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when Y may be hydrogen, A may be selected from the group consisting of: optionally substituted azetidinyl-Y and optionally substituted piperidinyl-Y For example, provided is a compound comprising a structure shown as formula (II), formula (II-a1), formula (II-a2), formula (II-b1), or formula (II-b2) or a tautomer, a mesomer, a racemate, an enantiomer or a diastereoisomer thereof, or a mixture thereof, or a pharmaceutically acceptable salt thereof, wherein
when Y may be hydrogen, A may be selected from the group consisting of: optionally substituted

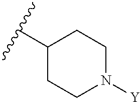

and optionally substituted

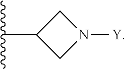

For example, provided is a compound comprising a structure shown as formula (II), formula (II-a1), formula (II-a2), formula (II-b1), or formula (II-b2), or a tautomer, a mesomer, a racemate, an enantiomer or a diastereoisomer thereof, or a mixture thereof, or a pharmaceutically acceptable salt thereof, wherein the compound comprises a structure shown as formula (II-a1-1), (II-a1-2), (II-a1-3), (II-a2-1), (II-a2-2) or (II-a2-3),

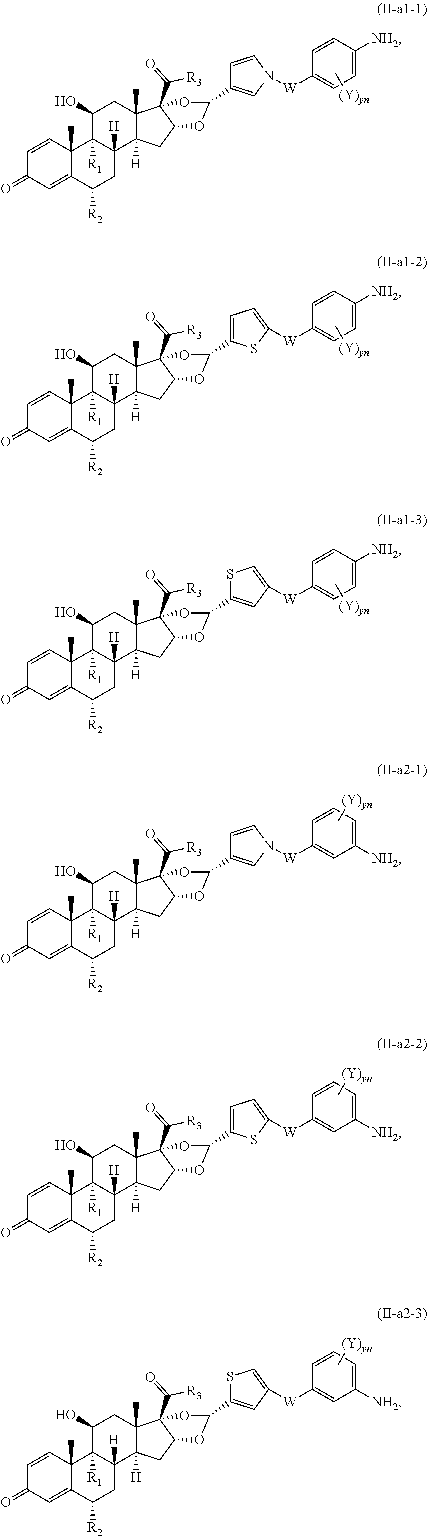

wherein,
R₁ and R₂ are each independently selected from: hydrogen, protium, deuterium, tritium, halogen, and C₁-C₆ alkyl;
R₃ is selected from: —CH₂Cl, —CH₂SH, —CH₂OH,

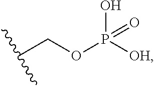 [SMILES: *COP(=O)(O)O]

OCH₃, —OCH₂F, —OCH₂Cl, —OCH₂CN, —OCH₂CH₃, —SH, —SCH₂F, —SCH₂Cl, —SCH₂CF₃ and —SCH₂CN;
in the general formulas (II-a1-1) and (II-a2-1), Y is each independently selected from: hydrogen, halogen, hydroxy, sulfhydryl, amino, C₁-C₆ alkyl, and —OC₁-C₆ alkyl; in the general formulas (II-a1-2), (II-a1-3), (II-a2-2) and (II-a2-3), Y is each independently selected from: hydrogen, halogen, hydroxy, sulfhydryl, amino, C₁-C₆ alkyl, —OC₁-C₆ alkyl and —C₁-C₆ alkylhydroxy;
yn is selected from 0, 1 or 2;
W is selected from

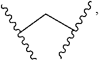 [SMILES: *C*]

### CH(CH₃)—, and

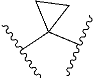 [SMILES: *C1(*)CC1]

For example, provided is a compound comprising a structure shown as formula (II), formula (II-a1), formula (II-a2), formula (II-b1), or formula (II-b2), or a tautomer, a mesomer, a racemate, an enantiomer or a diastereoisomer thereof, or a mixture thereof, or a pharmaceutically acceptable salt thereof, wherein the compound comprises a structure shown as formula (II-a1-1x), (II-a1-2x), (II-a1-3x), (II-a2-1x), (II-a2-2x) or (II-a2-3x),

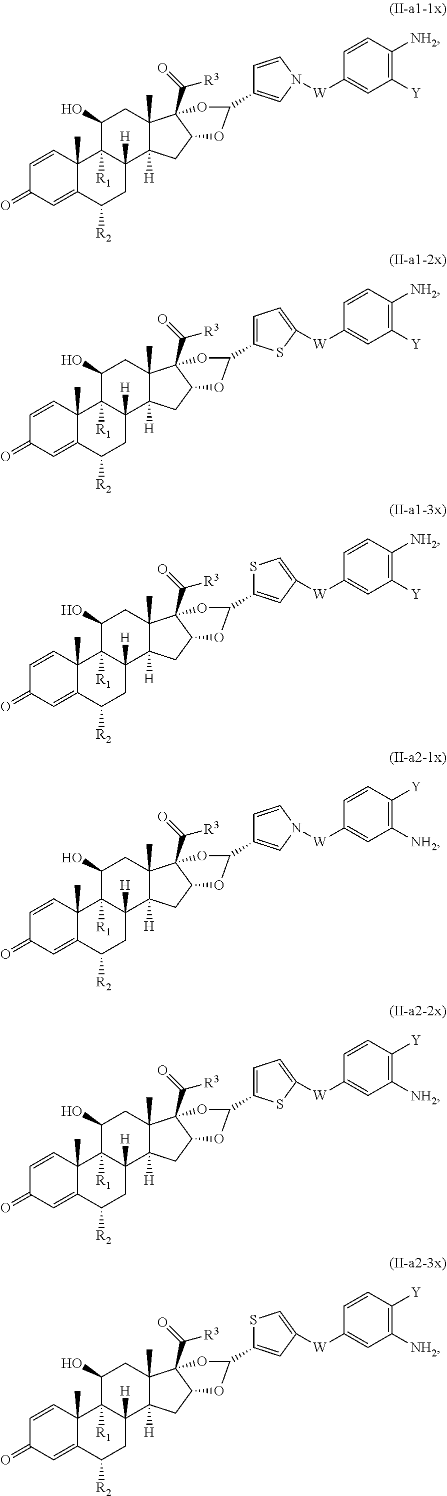

wherein,
R₁ and R₂ are each independently selected from: hydrogen, protium, deuterium, tritium, halogen, and C₁-C₆ alkyl;
R₃ is selected from: —CH₂Cl, —CH₂SH, —CH₂OH,

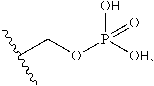 [SMILES: *COP(=O)(O)O]

—OCH₃, —OCH₂F, —OCH₂Cl, —OCH₂CN, —OCH₂CH₃, —SH, —SCH₂F, —SCH₂Cl, —SCH₂CF₃ and —SCH₂CN;
in the general formulas (II-a1-1x) and (II-a2-1x), Y is each independently selected from: hydrogen, halogen, hydroxy, sulfhydryl, amino, C₁-C₆ alkyl, and —OC₁-C₆ alkyl; in the general formulas (II-a1-2x), (II-a1-3x), (II-a2-2x) and (II-a2-3x), Y is each independently selected from: hydrogen, halogen, hydroxy, sulfhydryl, amino, C₁-C₆ alkyl, —OC₁-C₆ alkyl and —C₁-C₆ alkylhydroxy;
W is selected from

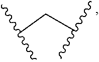 [SMILES: *C*]

### CH(CH₃)—, and

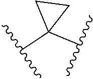 [SMILES: *C1(*)CC1]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1) or formula (II-a2):

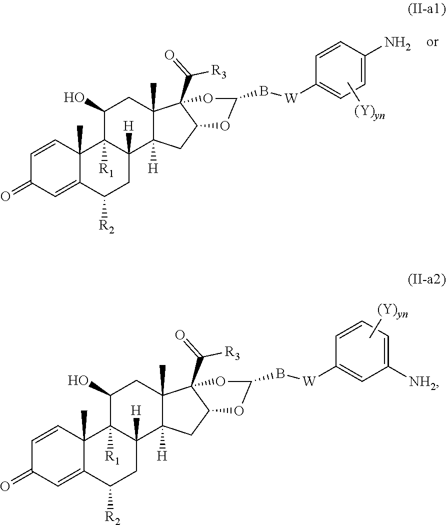

wherein

R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

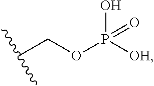 [SMILES: *COP(=O)(O)O]

optionally substituted

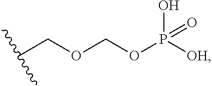 [SMILES: *COCOP(=O)(O)O]

optionally substituted

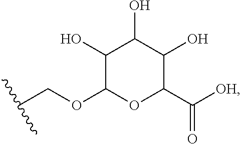 [SMILES: *COC1OC(C(=O)O)C(O)C(O)C1O]

optionally substituted

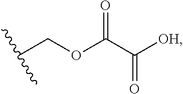 [SMILES: *COC(=O)C(=O)O]

optionally substituted

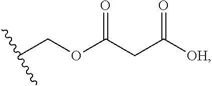 [SMILES: *COC(=O)CC(=O)O]

optionally substituted

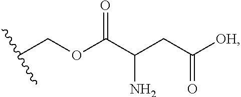 [SMILES: *COC(=O)C(N)CC(=O)O]

optionally substituted

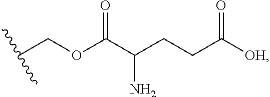 [SMILES: *COC(=O)C(N)CCC(=O)O]

optionally substituted

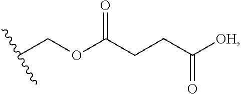 [SMILES: *COC(=O)CCC(=O)O]

optionally substituted

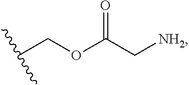 [SMILES: *COC(=O)CN]

optionally substituted

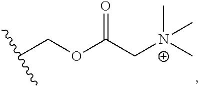 [SMILES: *COC(=O)C[N+](C)(C)C]

optionally substituted

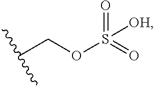 [SMILES: *COS(=O)(=O)O]

optionally substituted

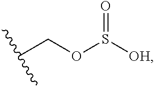 [SMILES: *COS(=O)O]

optionally substituted

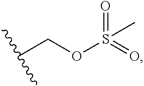 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

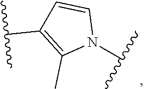 [SMILES: *c1ccn(*)c1C]

optionally substituted

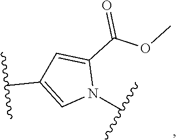 [SMILES: *c1cc(C(=O)OC)n(*)c1]

optionally substituted

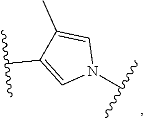 [SMILES: *c1cn(*)cc1C]

optionally substituted

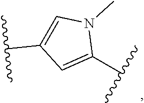 [SMILES: *c1cc(*)n(C)c1]

optionally substituted

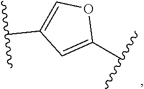 [SMILES: *c1coc(*)c1]

optionally substituted

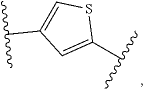 [SMILES: *c1csc(*)c1]

optionally substituted

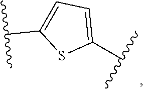 [SMILES: *c1ccc(*)s1]

optionally substituted

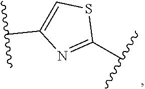 [SMILES: *c1csc(*)n1]

and optionally substituted

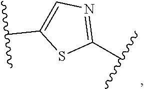 [SMILES: *c1cnc(*)s1]

W may be absent or W may be selected from the group consisting of the following structures that are optionally substituted:

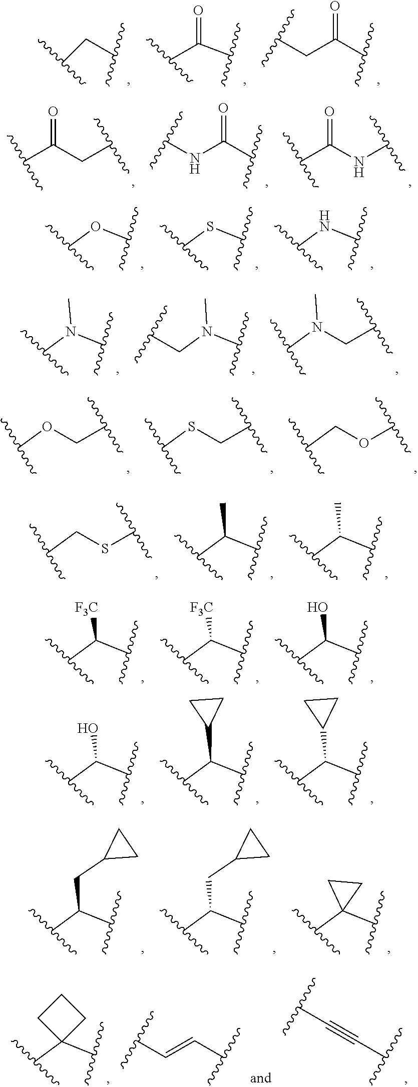

Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1) or formula (II-a2):

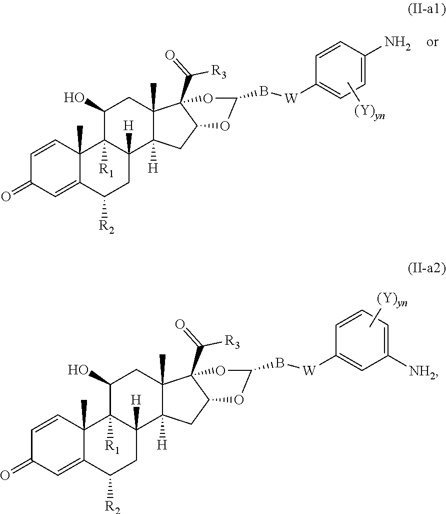

wherein
R₁ may be selected from the group consisting of: hydrogen and halogen, R₂ may be selected from the group consisting of: hydrogen and halogen, R₃ may be selected from the group consisting of: —CH₂OH, —SCH₂-halogen,

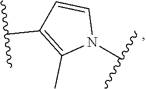 [SMILES: *c1ccn(*)c1C]

B may be selected from e t group consisting of: optionally substituted

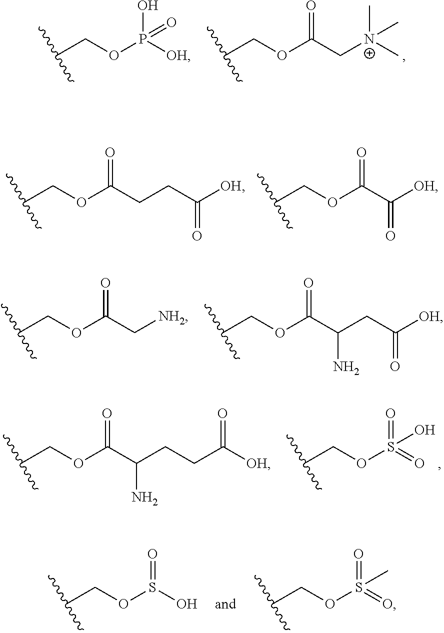

optionally substituted

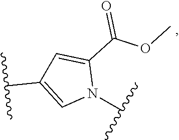 [SMILES: *c1cc(C(=O)OC)n(*)c1]

optionally substituted

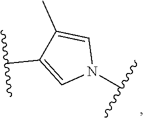 [SMILES: *c1cn(*)cc1C]

optionally substituted

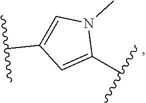 [SMILES: *c1cc(*)n(C)c1]

optionally substituted

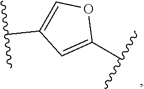 [SMILES: *c1coc(*)c1]

optionally substituted

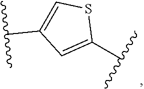 [SMILES: *c1csc(*)c1]

optionally substituted

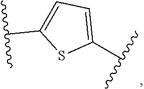 [SMILES: *c1ccc(*)s1]

optionally substituted

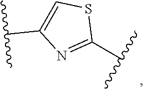 [SMILES: *c1csc(*)n1]

and optionally substituted

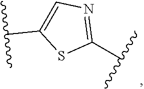 [SMILES: *c1cnc(*)s1]

W may be absent or W may be selected from the group consisting of the following structures that are optionally substituted: optionally substituted —CH₂—, optionally substituted —CH(CH₃)—, and optionally substituted

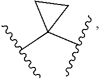 [SMILES: *C1(*)CC1]

Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1) or formula (II-b2):

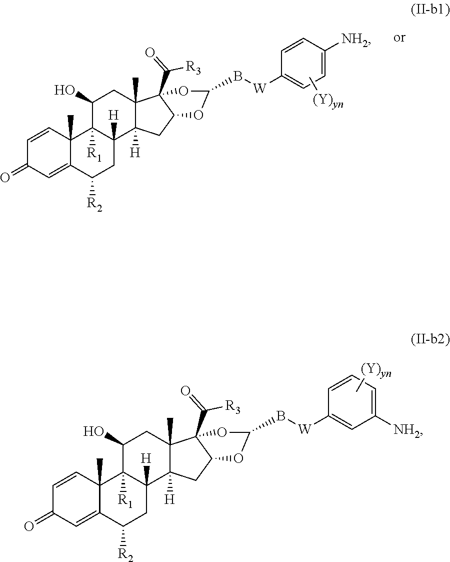

wherein
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

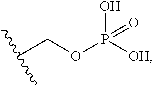 [SMILES: *COP(=O)(O)O]

optionally substituted

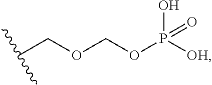 [SMILES: *COCOP(=O)(O)O]

optionally substituted

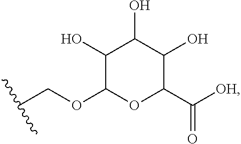 [SMILES: *COC1OC(C(=O)O)C(O)C(O)C1O]

optionally substituted

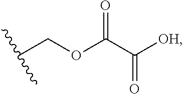 [SMILES: *COC(=O)C(=O)O]

optionally substituted

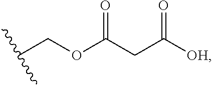 [SMILES: *COC(=O)CC(=O)O]

optionally substituted

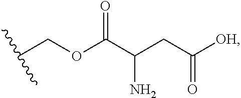 [SMILES: *COC(=O)C(N)CC(=O)O]

optionally substituted

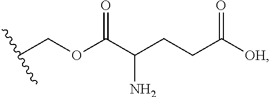 [SMILES: *COC(=O)C(N)CCC(=O)O]

optionally substituted

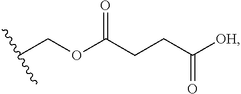 [SMILES: *COC(=O)CCC(=O)O]

optionally substituted

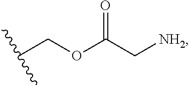 [SMILES: *COC(=O)CN]

optionally substituted

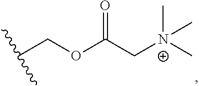 [SMILES: *COC(=O)C[N+](C)(C)C]

optionally substituted

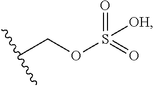 [SMILES: *COS(=O)(=O)O]

optionally substituted

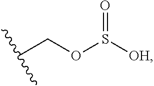 [SMILES: *COS(=O)O]

optionally substituted

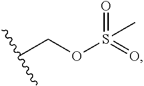 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

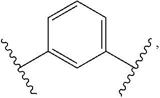 [SMILES: *c1cccc(*)c1]

optionally substituted

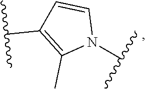 [SMILES: *c1ccn(*)c1C]

optionally substituted

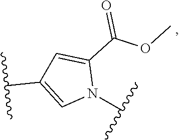 [SMILES: *c1cc(C(=O)OC)n(*)c1]

optionally substituted

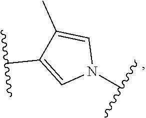 [SMILES: *c1cn(*)cc1C]

optionally substituted

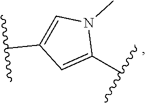 [SMILES: *c1cc(*)n(C)c1]

optionally substituted

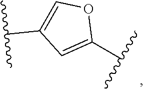 [SMILES: *c1coc(*)c1]

optionally substituted

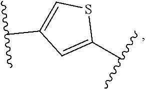 [SMILES: *c1csc(*)c1]

optionally substituted

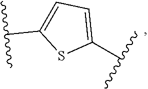 [SMILES: *c1ccc(*)s1]

optionally substituted

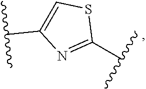 [SMILES: *c1csc(*)n1]

and optionally substituted

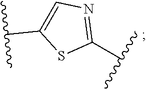 [SMILES: *c1cnc(*)s1]

W may be absent or W may be selected from the group consisting of the following structures that are optionally substituted:

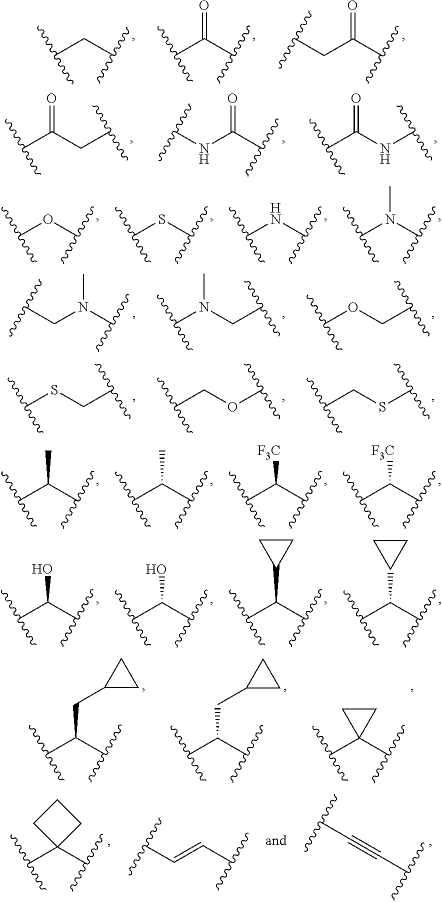

wherein when B may be optionally substituted

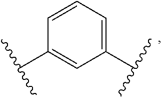 [SMILES: *c1cccc(*)c1]

### W may not be —S—;

Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1) or formula (II-b2):

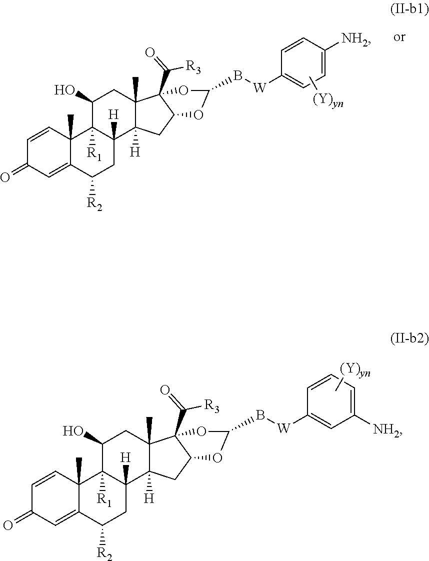

wherein
R₁ may be selected from the group consisting of: hydrogen and halogen, R₂ may be selected from the group consisting of: hydrogen and halogen, R₃ may be selected from the group consisting of: —CH₂OH, —SCH₂-halogen,

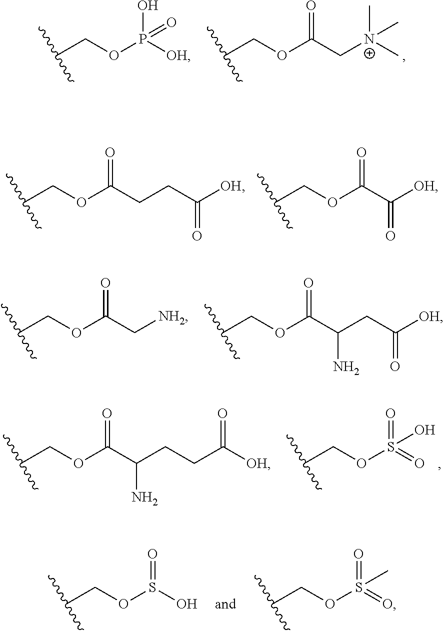

B may be selected from the group consisting of: optionally substituted

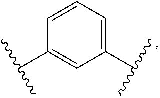 [SMILES: *c1cccc(*)c1]

optionally substituted

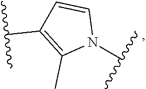 [SMILES: *c1ccn(*)c1C]

optionally substituted

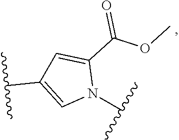 [SMILES: *c1cc(C(=O)OC)n(*)c1]

optionally substituted

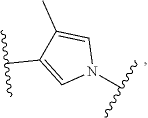 [SMILES: *c1cn(*)cc1C]

optionally substituted

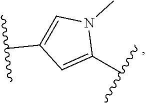 [SMILES: *c1cc(*)n(C)c1]

optionally substituted

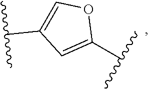 [SMILES: *c1coc(*)c1]

optionally substituted

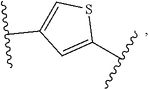 [SMILES: *c1csc(*)c1]

optionally substituted

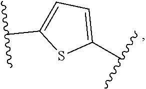 [SMILES: *c1ccc(*)s1]

optionally substituted

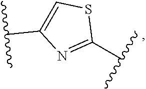 [SMILES: *c1csc(*)n1]

and optionally substituted

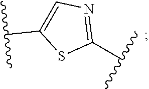 [SMILES: *c1cnc(*)s1]

W may be absent or W may be selected from the group consisting of the following structures that are optionally substituted: optionally substituted —CH₂—, optionally substituted —CH(CH₃)—, and optionally substituted

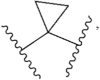 [SMILES: *C1(*)CC1]

Y may be H, and yn may be a number greater than 0.

The present application provides a compound or a tautomer, a mesomer, a racemate, an enantiomer or a diastereoisomer thereof, or a mixture thereof, or a pharmaceutically acceptable salt thereof,
wherein the compound comprises a structure that may be selected from the group consisting of:

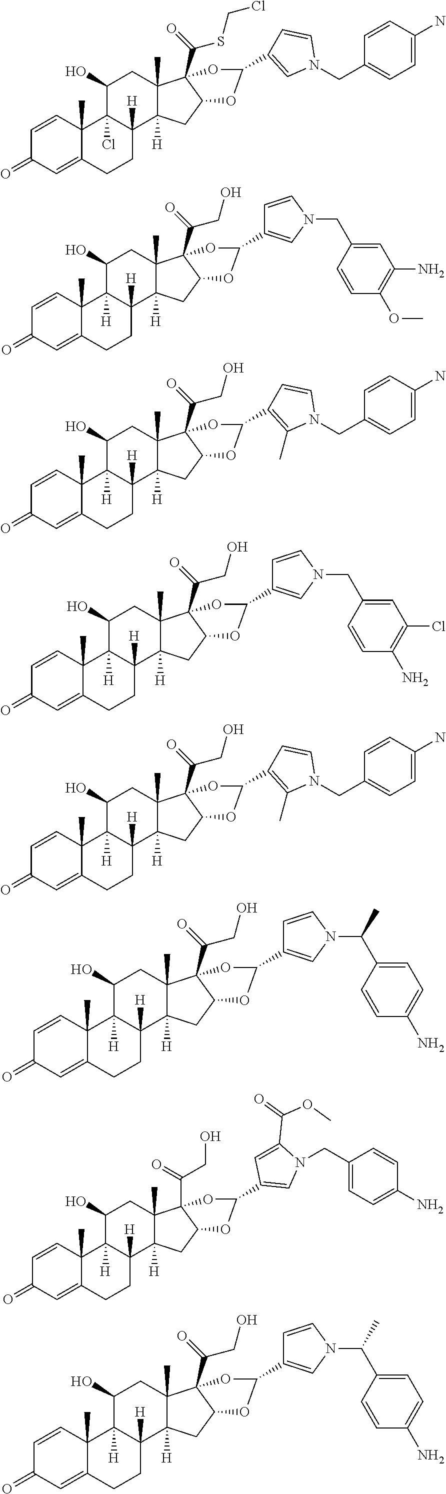

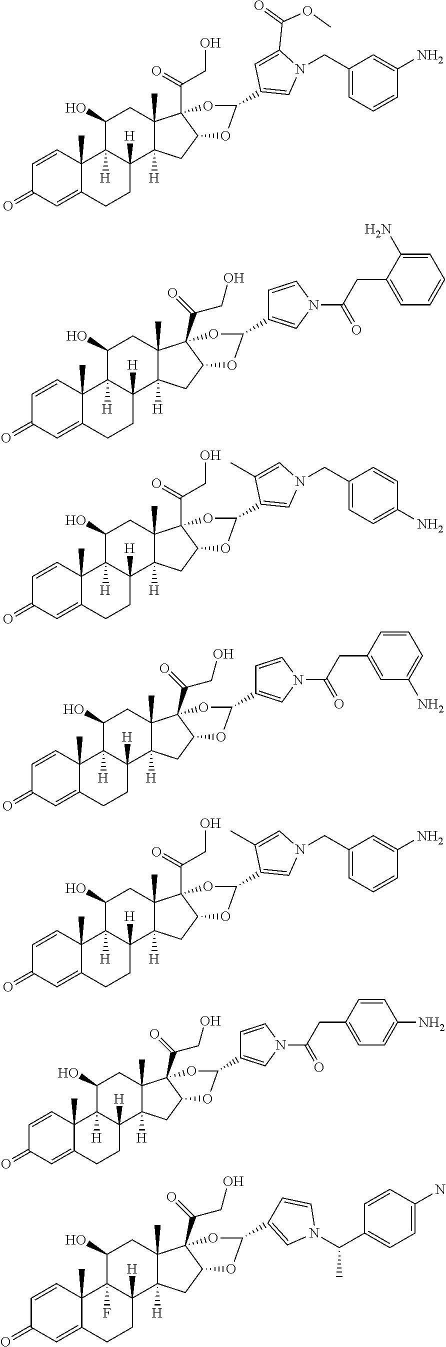

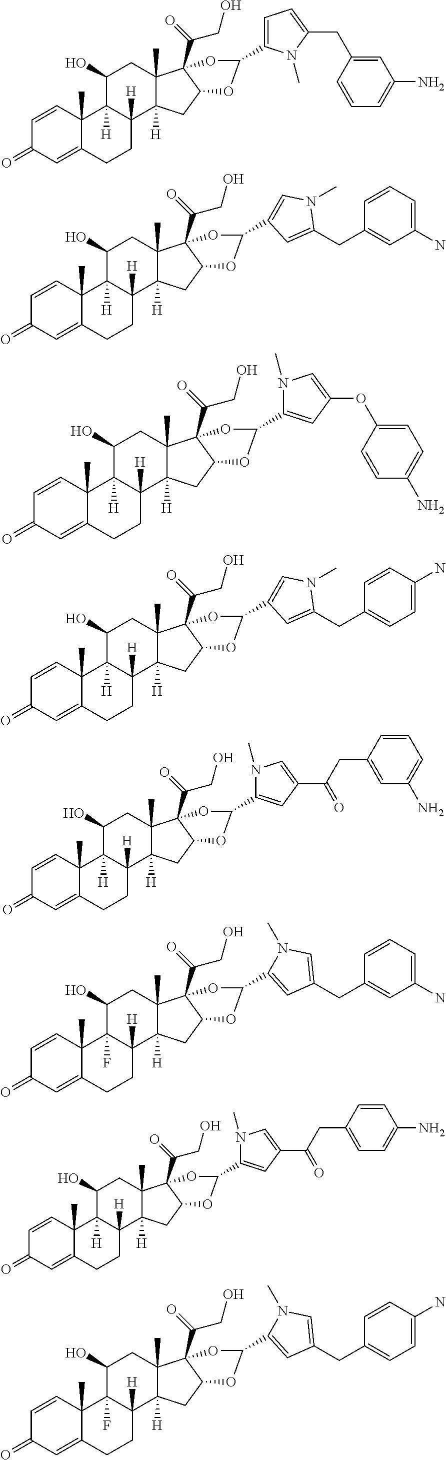

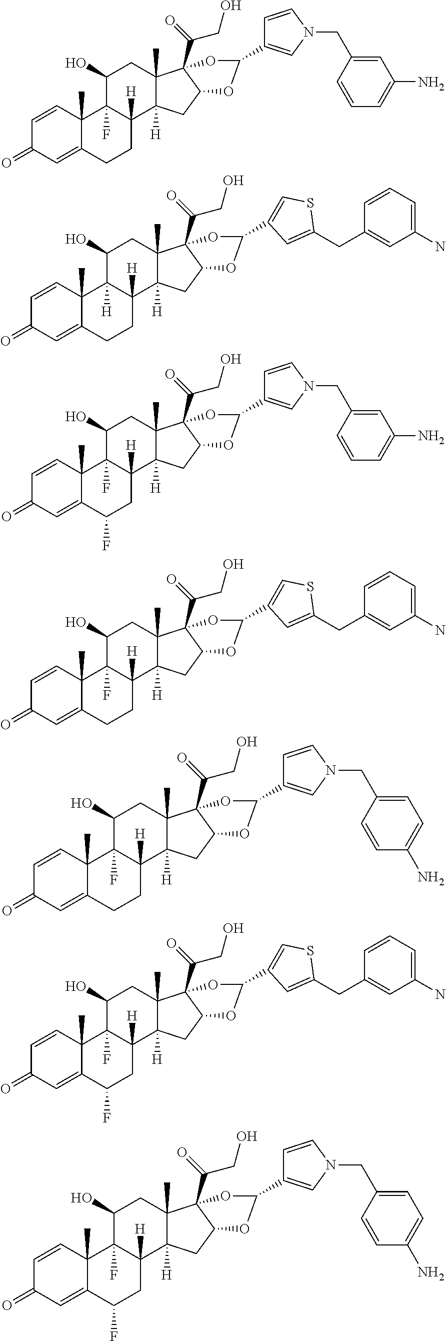

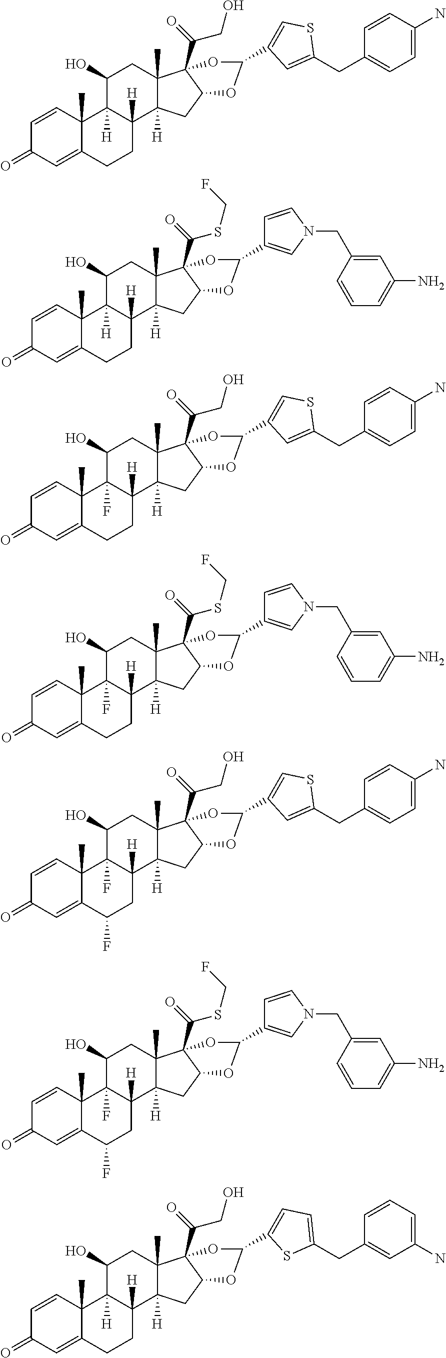

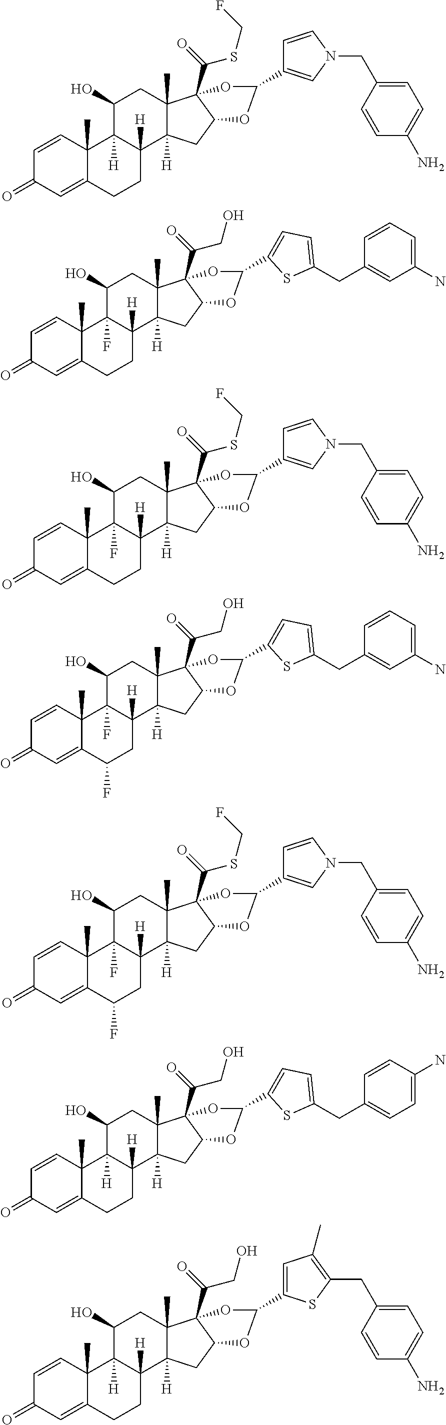

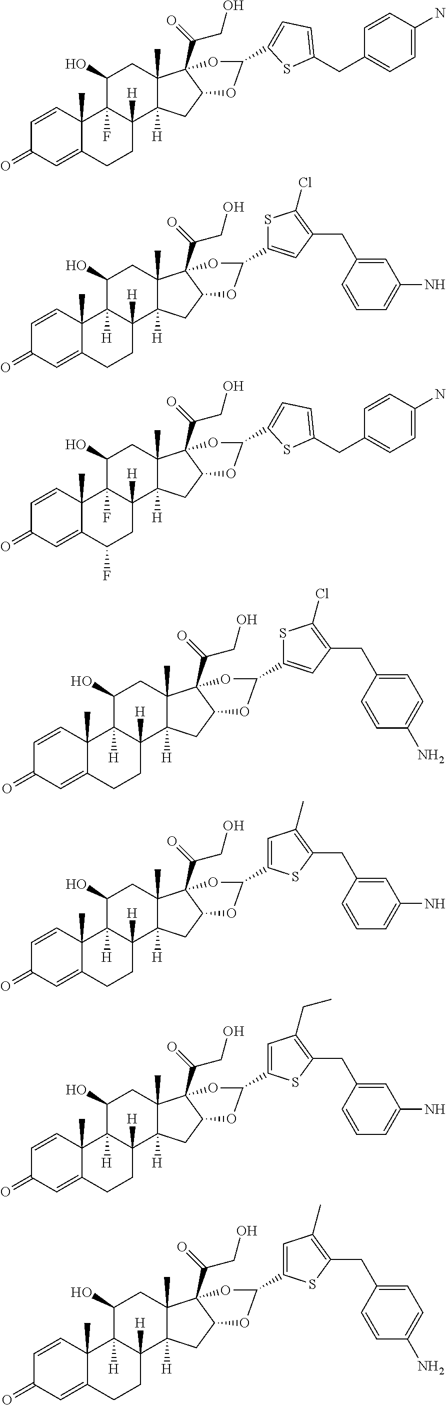

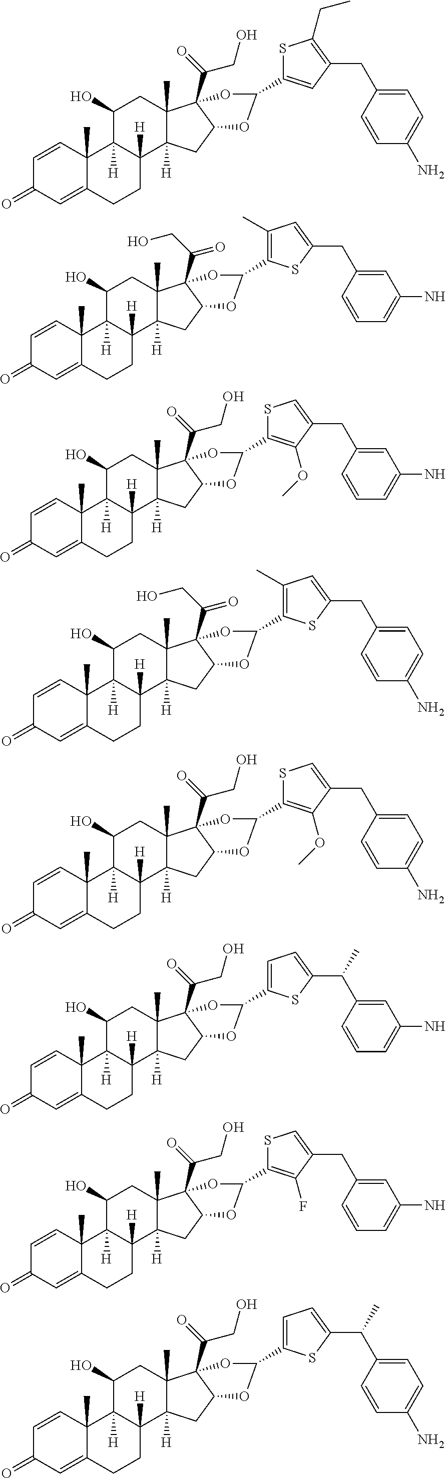

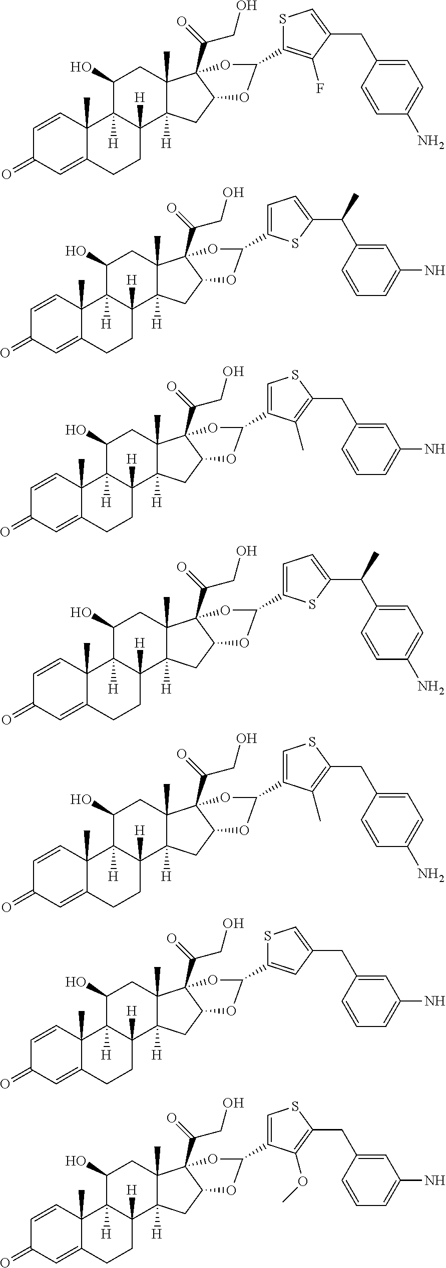

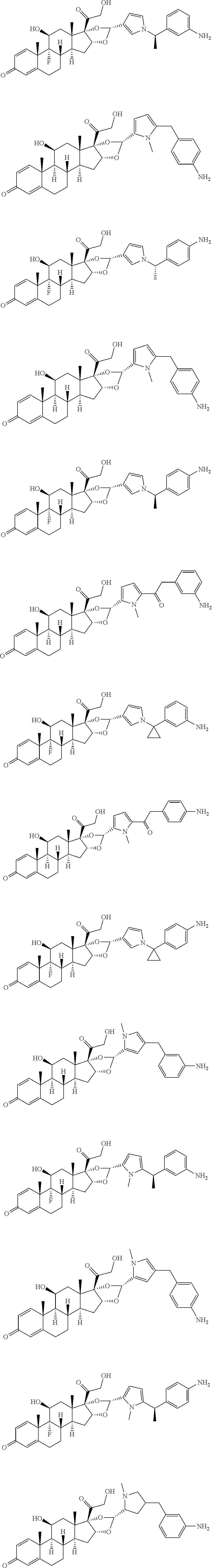

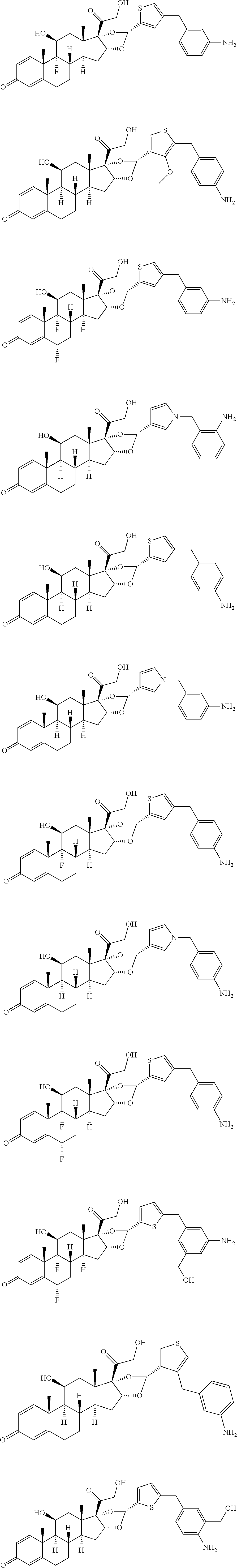

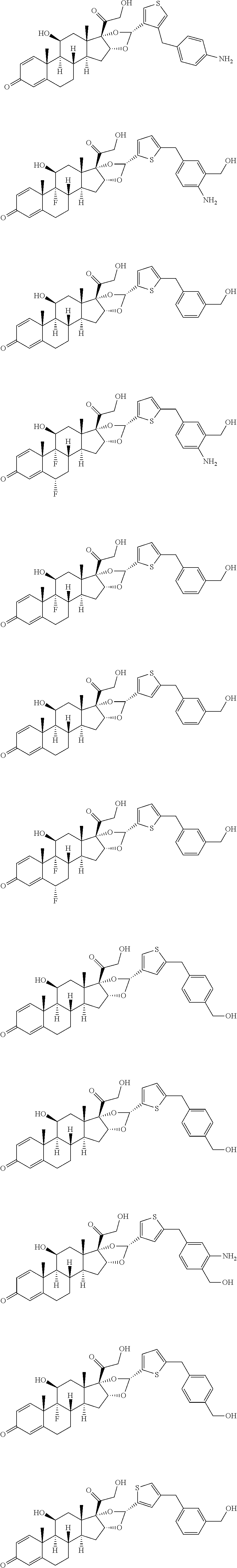

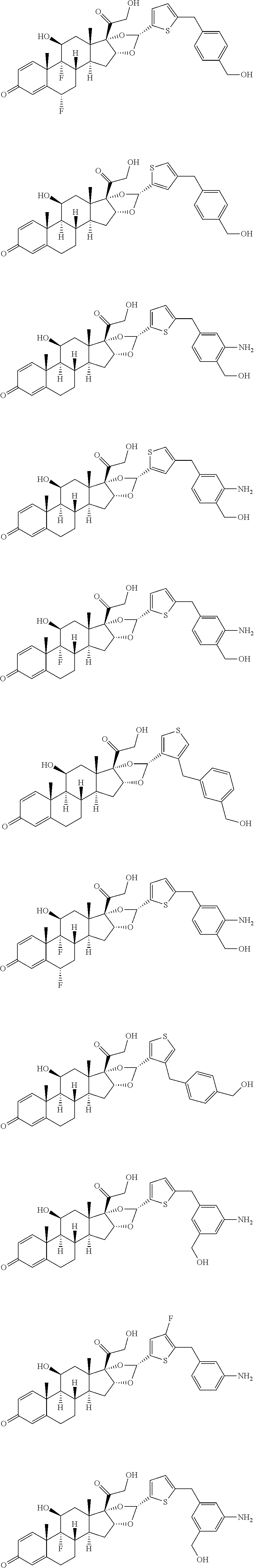

The present application provides a compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

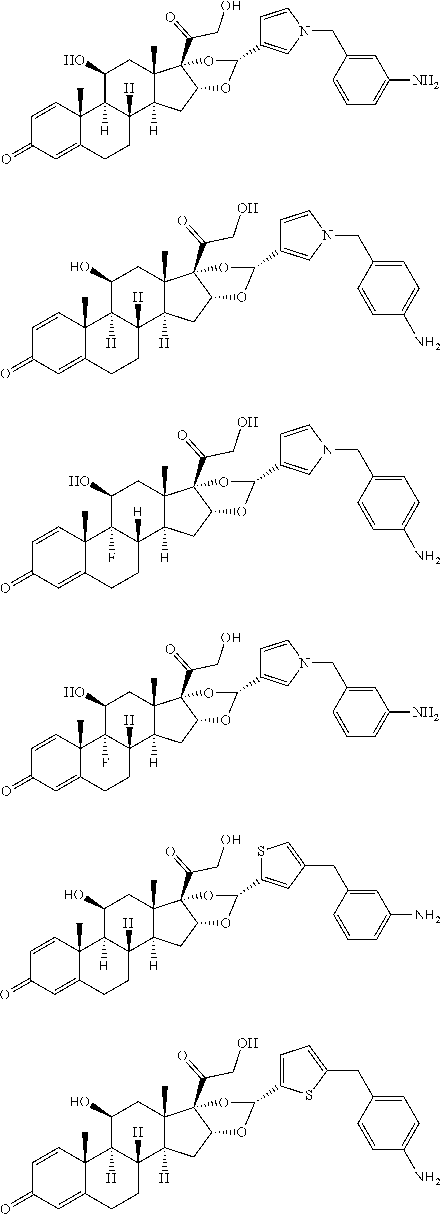

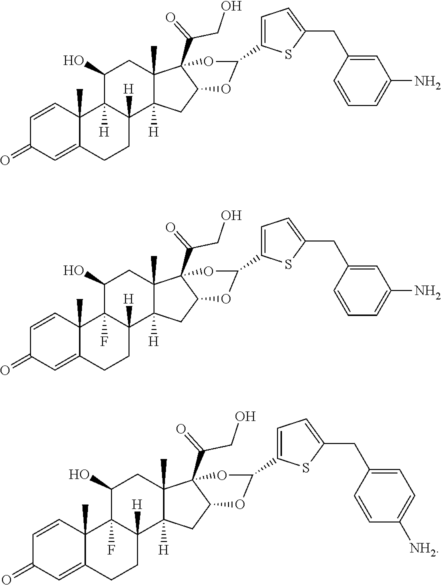

For example, the present application provides a compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

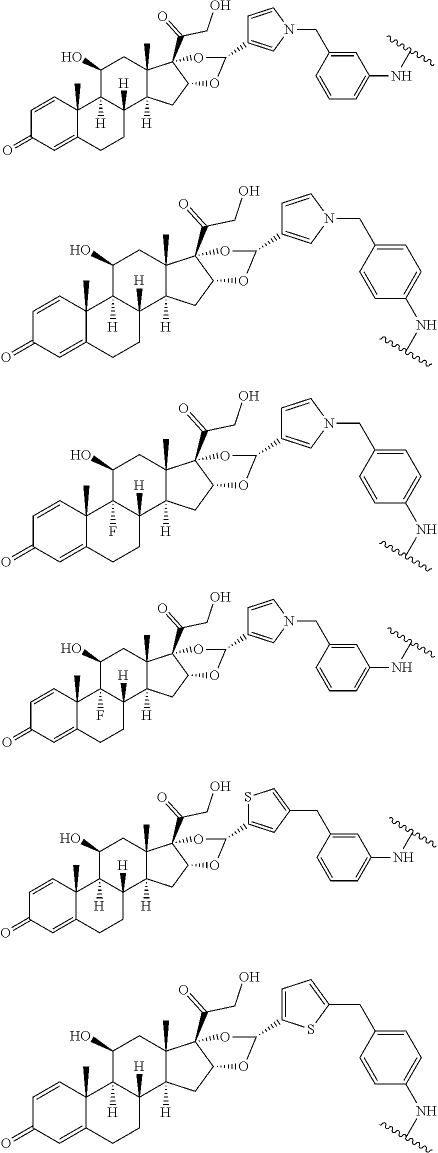

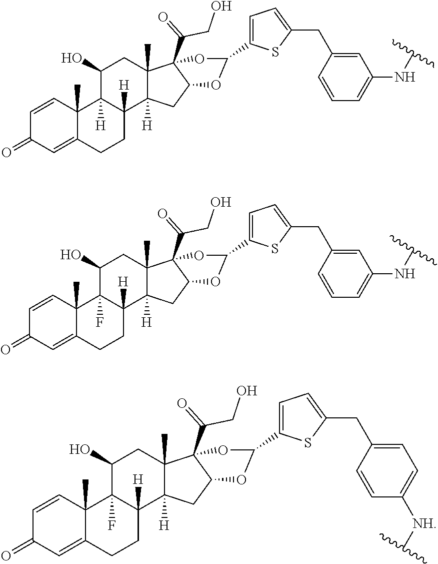

For example, the present application provides a compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

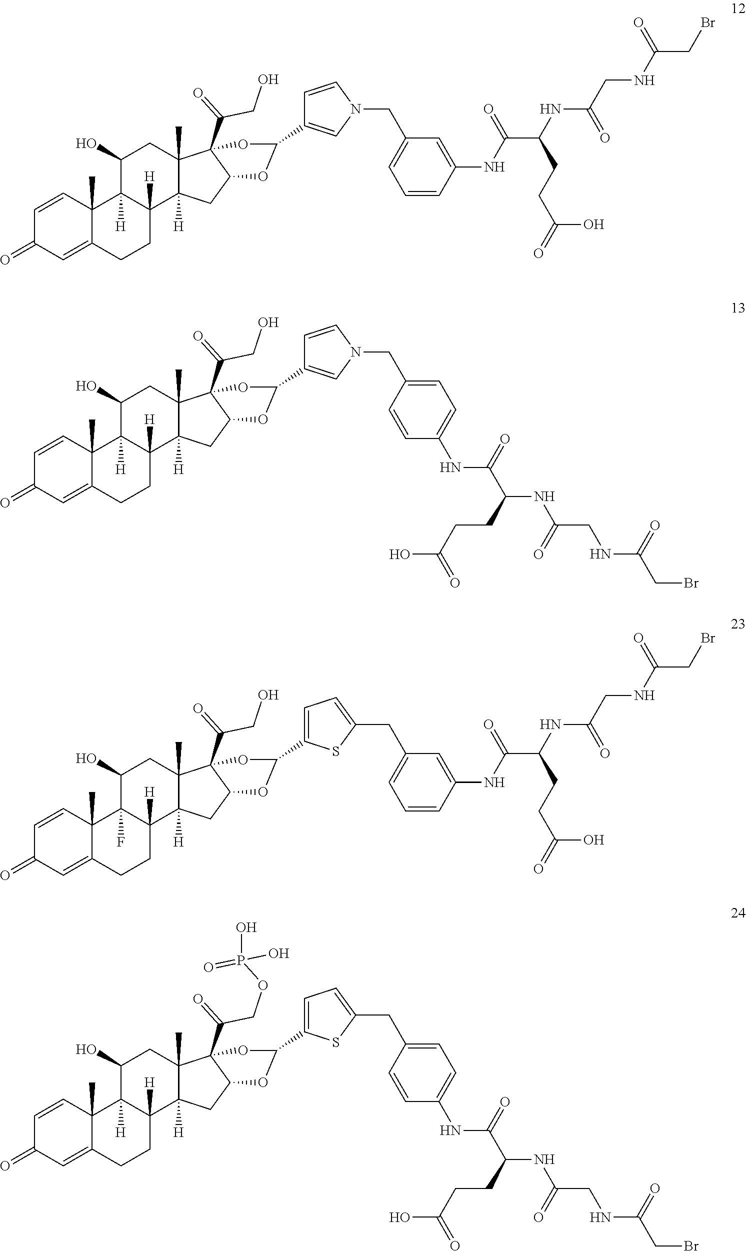

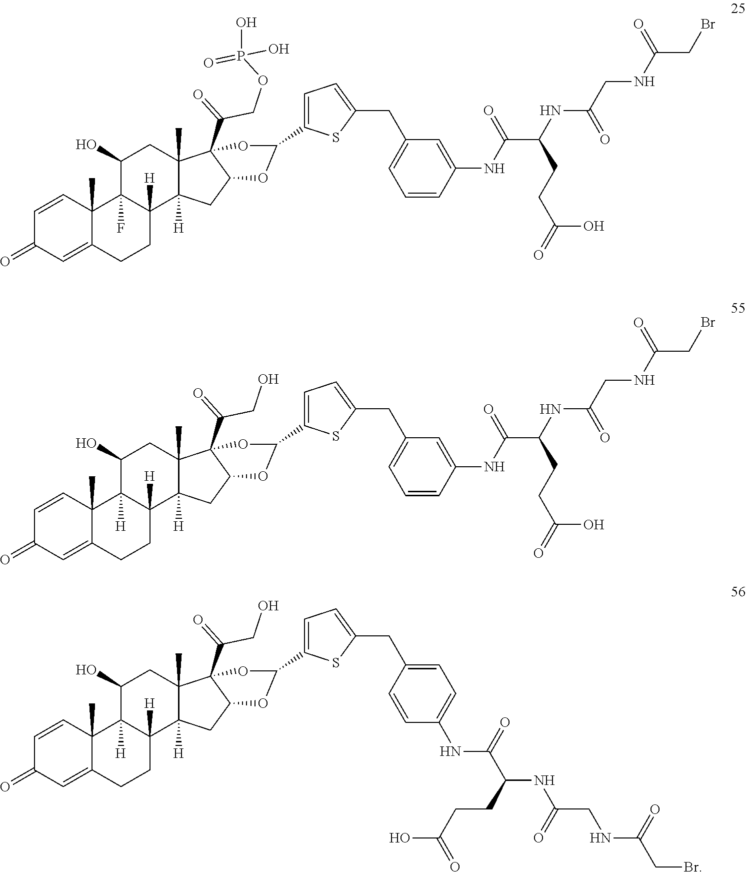

The present application provides a compound or a tautomer, a mesomer, a racemate, an enantiomer or a diastereoisomer thereof, or a mixture thereof, or a pharmaceutically acceptable salt thereof, wherein the compound comprises formulas (II-a1-C-1), (II-a1-C-2), (II-a1-C-3), (II-a2-C-1), (II-a2-C-2), and (II-a2-C-3),

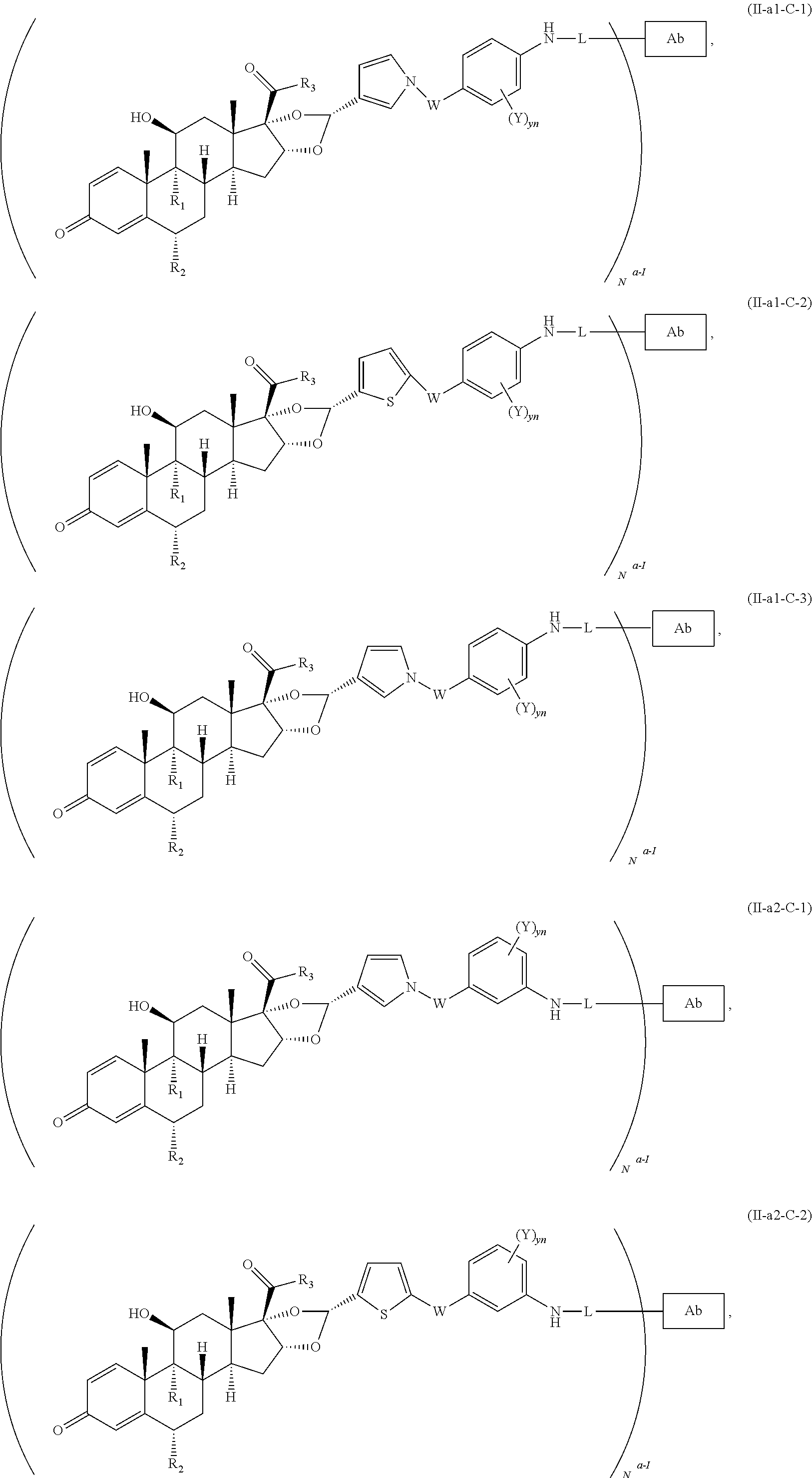

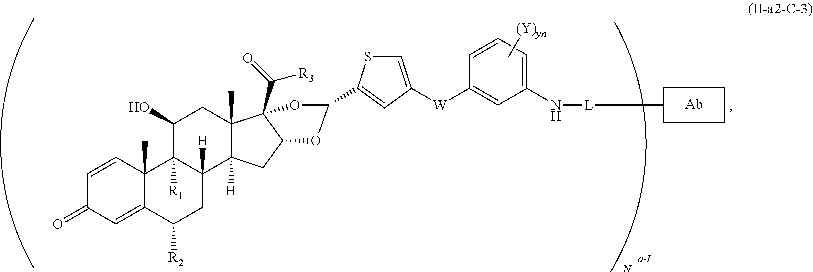

wherein,
R₁ and R₂ are each independently selected from: hydrogen, protium, deuterium, tritium, halogen, and C₁-C₆ alkyl;
R₃ is selected from: —CH₂Cl, —CH₂SH, —CH₂OH,

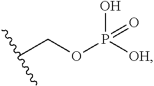 [SMILES: *COP(=O)(O)O]

—OCH₃, —OCH₂F, —OCH₂Cl, —OCH₂CN, —OCH₂CH₃, —SH, —SCH₂F, —SCH₂Cl, —SCH₂CF₃ and —SCH₂CN;
W is selected from

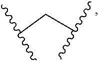 [SMILES: *C*]

### CH(CH₃)—, and

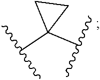 [SMILES: *C1(*)CC1]

in the formulas (II-a1-C-1) and (II-a2-C-1), Y is each independently selected from: hydrogen, halogen, hydroxy, sulfhydryl, amino, C₁-C₆ alkyl, and —OC₁-C₆ alkyl; in the formulas (II-a1-C-2), (II-a1-C-3), (II-a2-C-2) and (II-a2-C-2), Y is each independently selected from: hydrogen, halogen, hydroxy, sulfhydryl, amino, C₁-C₆ alkyl, —OC₁-C₆ alkyl and —C₁-C₆ alkylhydroxy;
yn is selected from 0, 1 or 2;
Ab is selected from an antibody or an antigen-binding fragment thereof;
L comprises -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

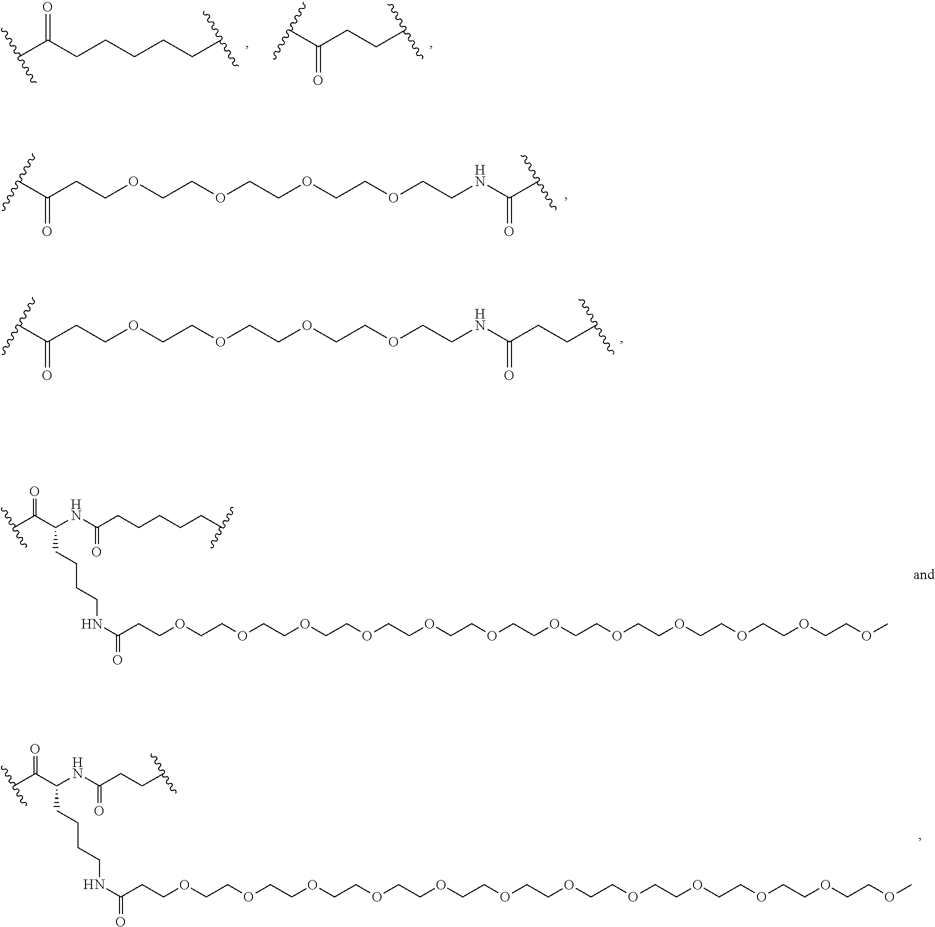 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

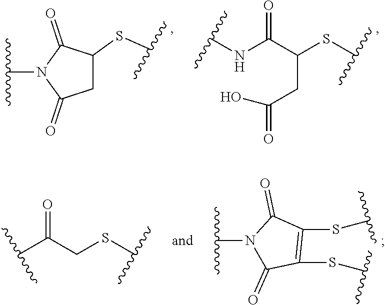

preferably L is

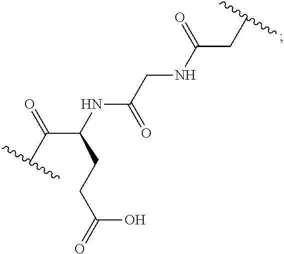 [SMILES: *CC(=O)NCC(=O)N[C@@H](CCC(=O)O)C(*)=O]

wherein N^{a-1} is a number from about 0 to about 20.

For example, the compound is selected from:

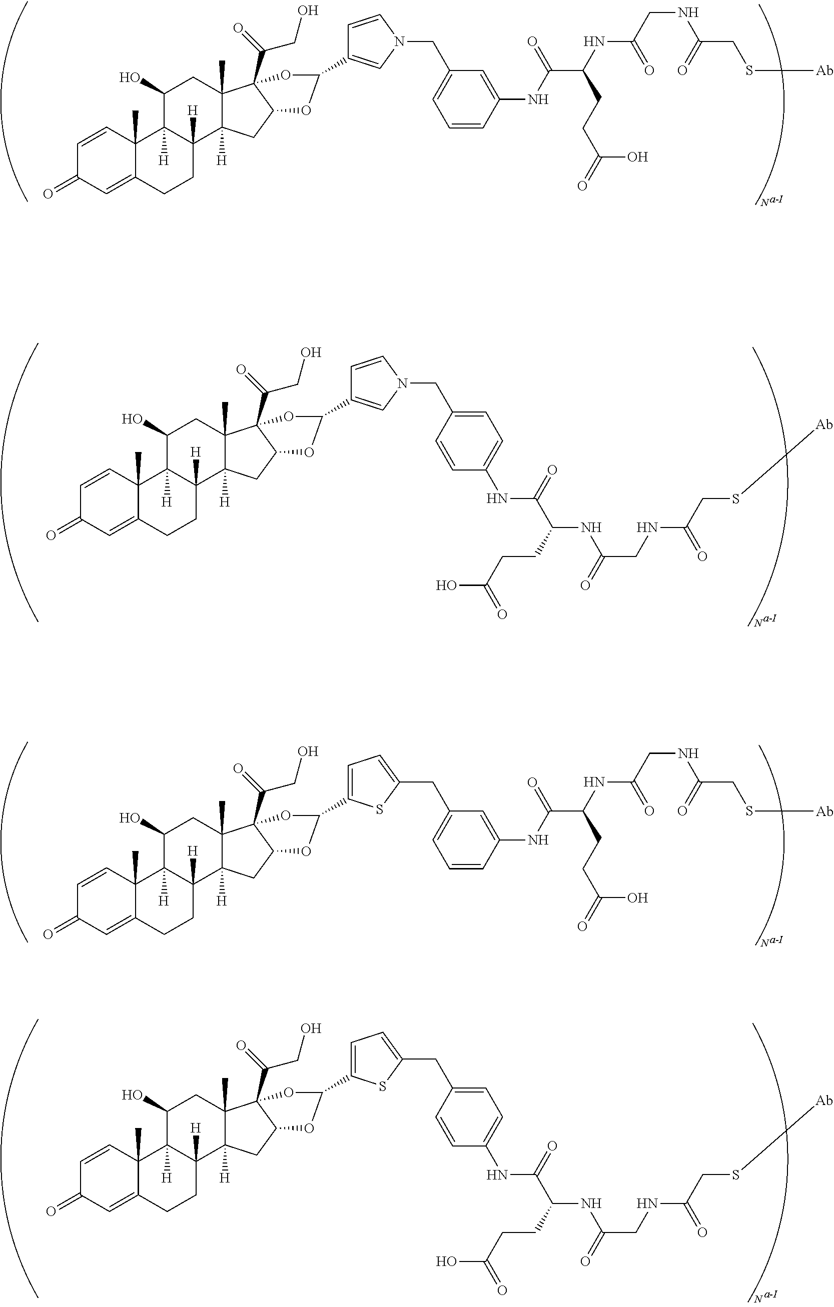

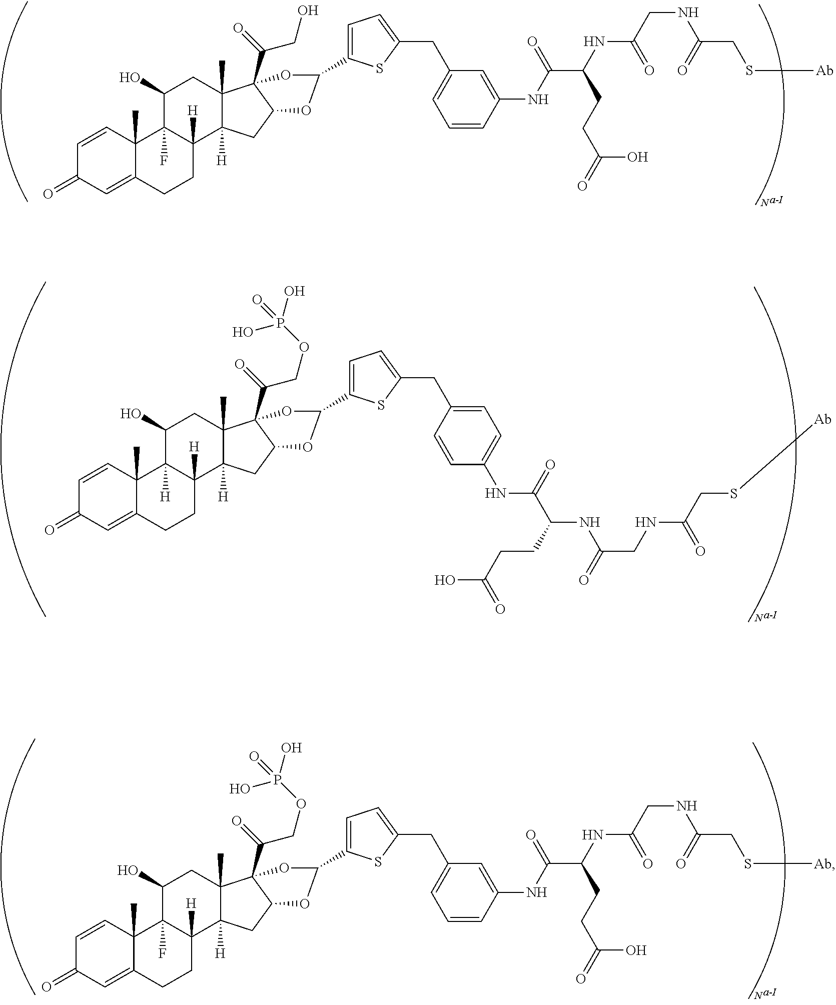

wherein N^{a-1} is a number from about 0 to about 20, and Ab is selected from an antibody or antigen-binding fragment thereof:

For example, the compound is selected from:

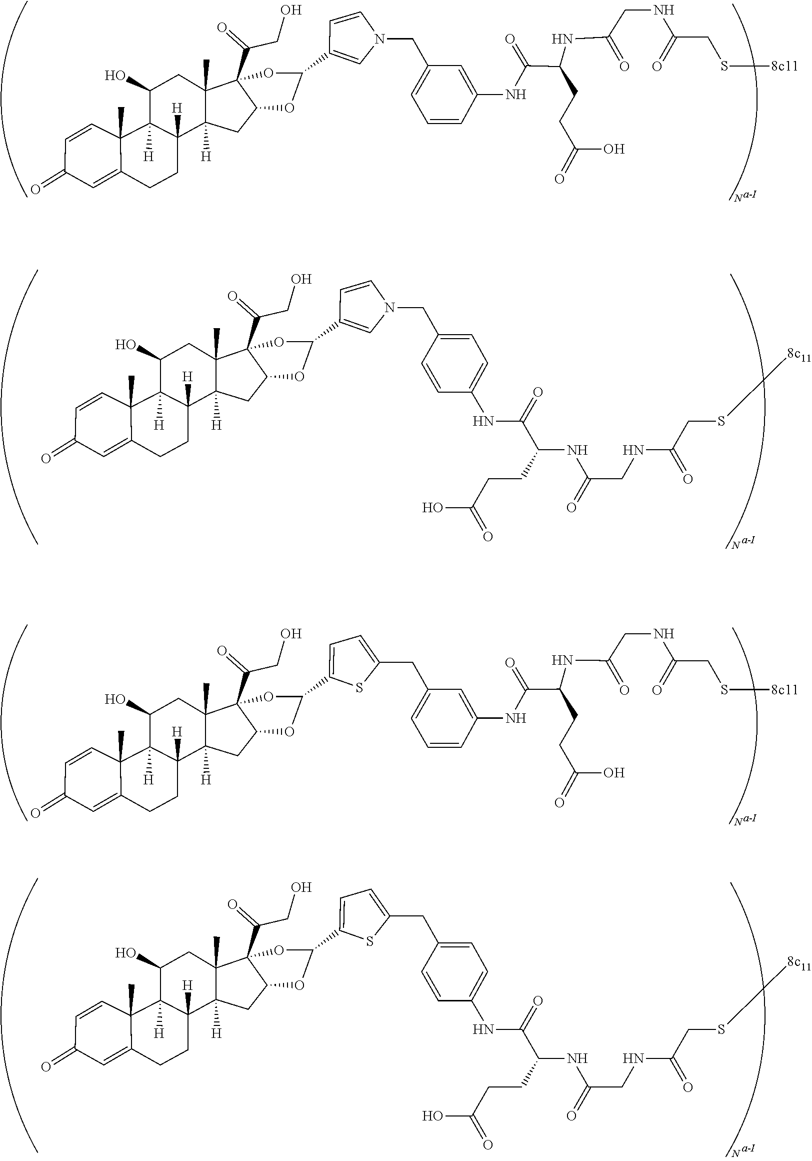

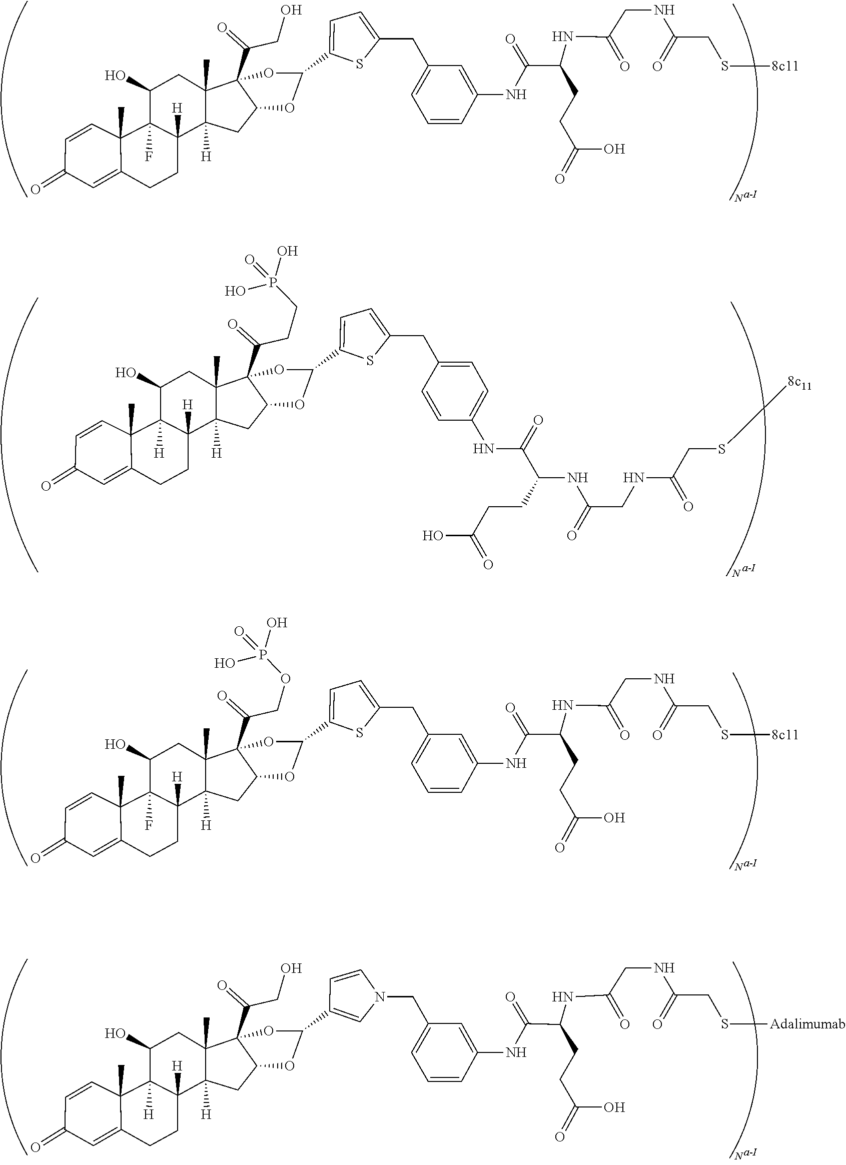

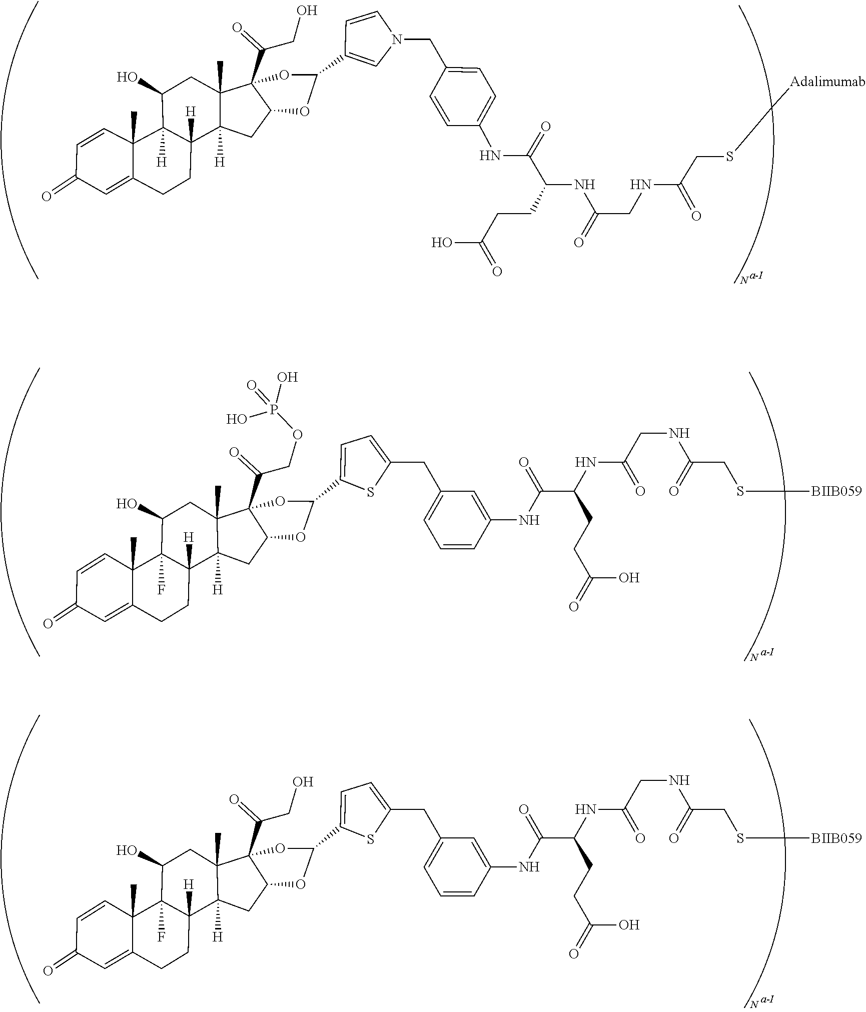

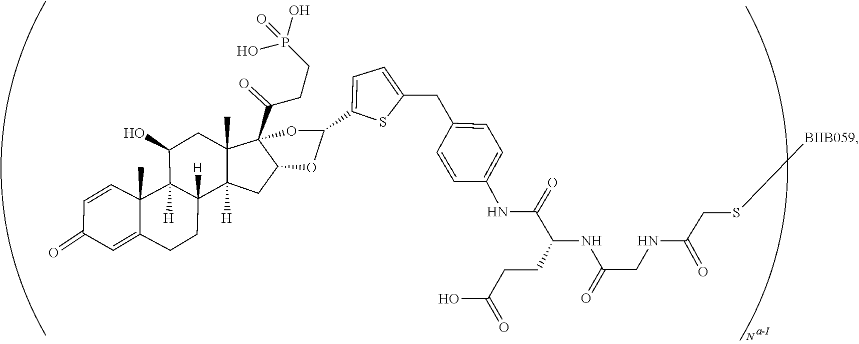

wherein N^{a-1} is a number from about 0 to about 20.

The present application provides a conjugate comprising the compound or the tautomer, the mesomer, the racemate, the enantiomer or the diastereoisomer thereof, or the mixture thereof, or the pharmaceutically acceptable salt thereof described herein.

For example, the conjugate of the present application comprises antibody-drug conjugates.

For example, the present application provides a conjugate, wherein an active metabolite of the conjugate comprises the compound described herein.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-A):

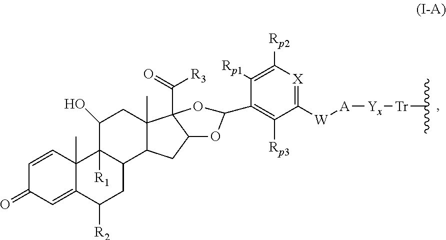

wherein

R₁, R₂, R₃, Rₚ₁, Rₚ₂, and Rₚ₃ may each independently be any group;
X may be selected from the group consisting of: N and optionally substituted CH;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; when X may be CH and W may be —S—, A may not be phenyl; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-A), formula (I-a2-A), formula (I-a3-A) or formula (I-a4-A):

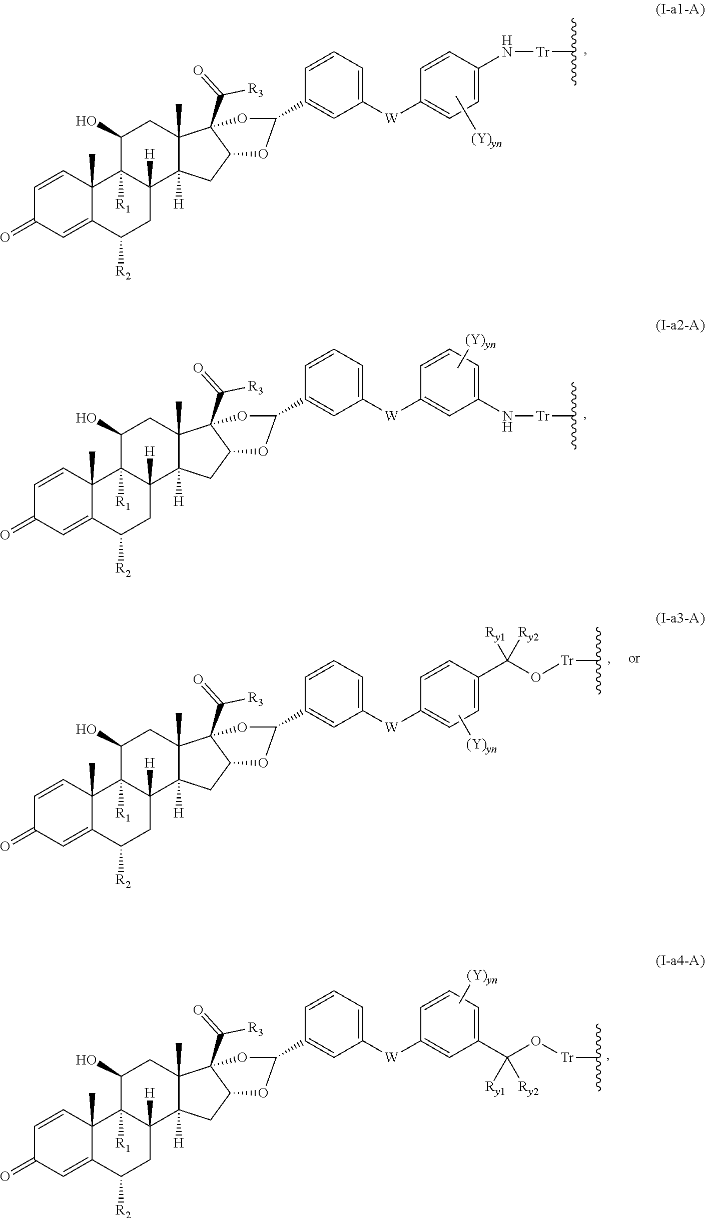

wherein
R₁, R₂, R₃, Ry₁, Ry₂ and Y may each independently be any group, yn may be a number equal to or greater than 0,
W may be absent or W may be any group, and Tr may be absent or may be any group, wherein
W may not be —S—.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-A):

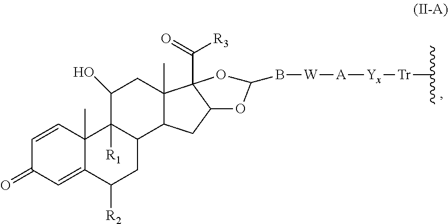

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-A), formula (II-a2-A), formula (II-a3-A) or formula (II-a4-A):

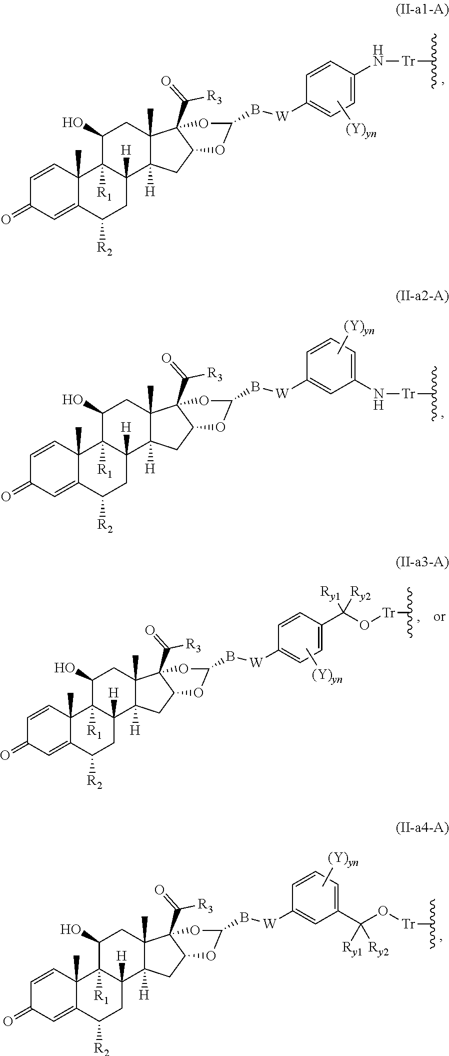

wherein
R₁, R₂, R₃, Ry₁, Ry₂, and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-A), formula (II-b2-A), formula (II-b3-A) or formula (II-b4-A):

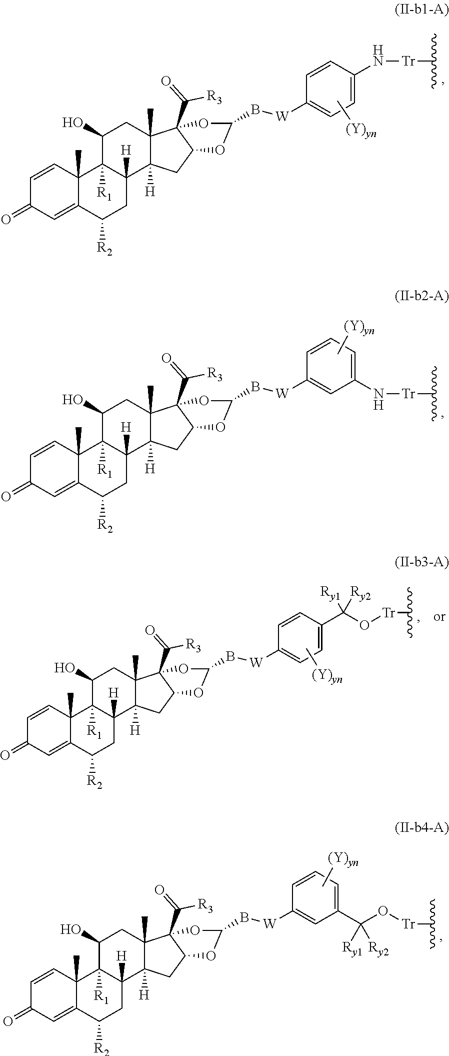

wherein
R₁, R₂, R₃, Ry₁, Ry₂ and Y may each independently be any group;
B may be selected from the group consisting of: optionally substituted

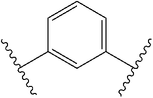 [SMILES: *c1cccc(*)c1]

and optionally substituted heteroarylene; for example, B may be selected from the group consisting of: optionally substituted

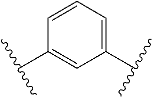 [SMILES: *c1cccc(*)c1]

and optionally substituted C₂-C₄ heteroarylene; W may be absent or W may be any group, wherein, when B may be optionally substituted

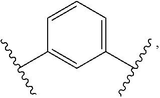 [SMILES: *c1cccc(*)c1]

W may not be —S—; yn may be a number equal to or greater than 0; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-B):

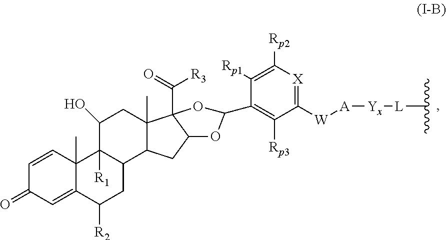

wherein
R₁, R₂, R₃, Rₚ₁, Rₚ₂, and Rₚ₃ may each independently be any group;
X may be selected from the group consisting of: N and optionally substituted CH;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; when X may be CH and W may be —S—, A may not be phenyl;
L may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-B), formula (I-a2-B), formula (I-a3-B) or formula (I-a4-B):

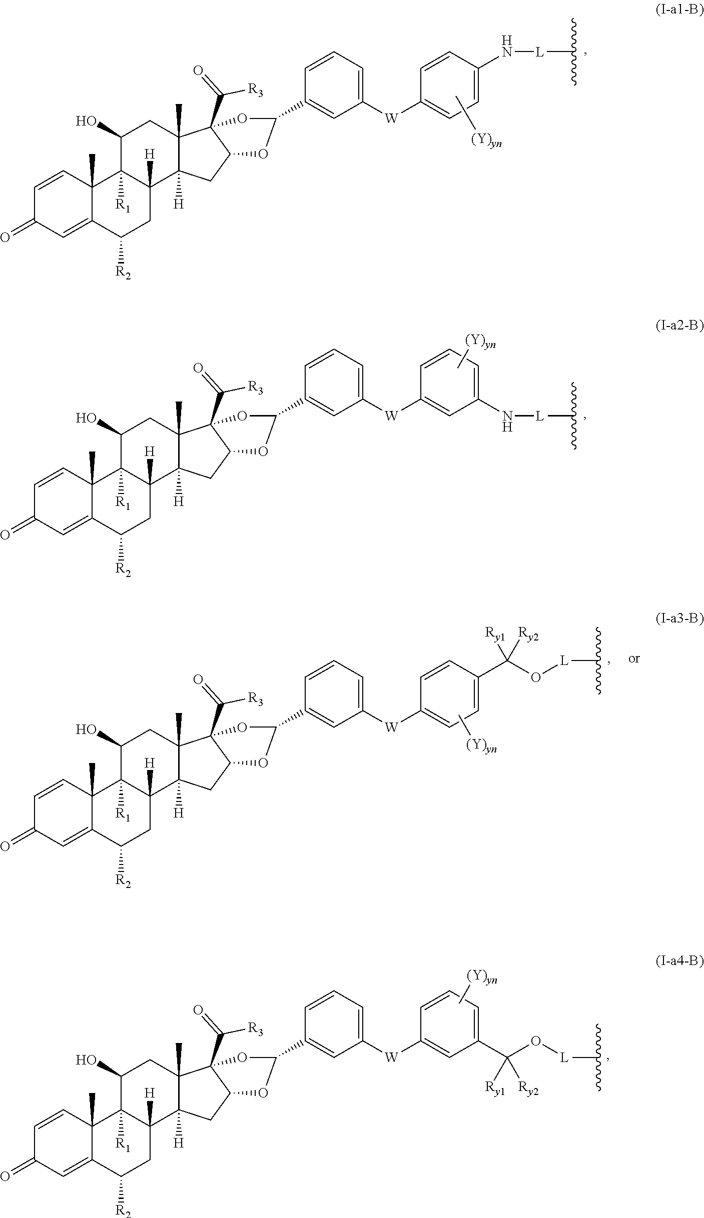

wherein
R₁, R₂, R₃, Ry₁, Ry₂ and Y may each independently be any group, yn may be a number equal to or greater than 0,
W may be absent or W may be any group, wherein W may not be —S—; L may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-B):

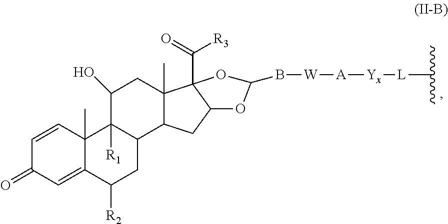

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; L may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-B), formula (II-a2-B), formula (II-a3-B) or formula (II-a4-B):

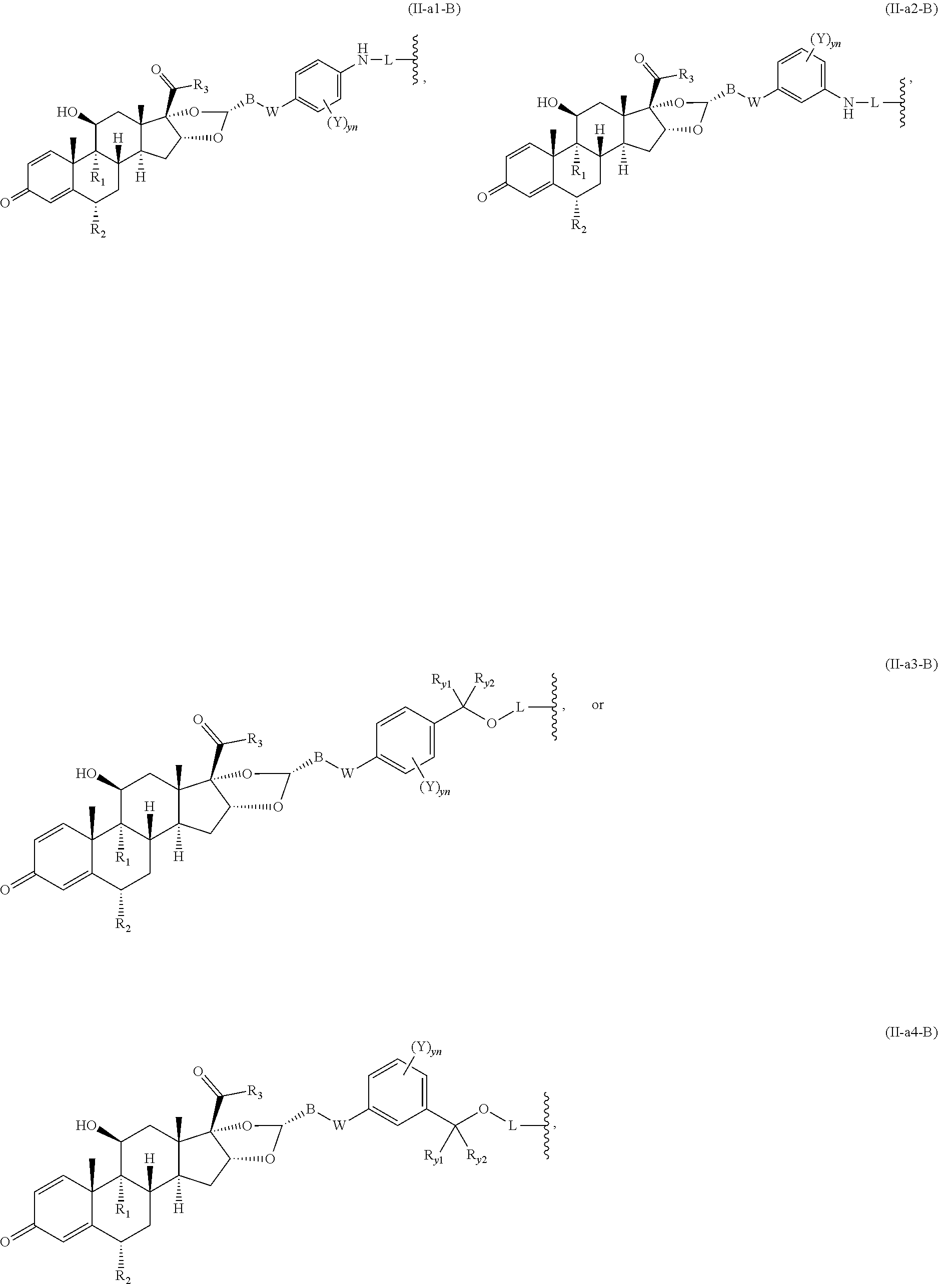

wherein
R₁, R₂, R₃, Ry₁, Ry₂, and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0; L may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-B), formula (II-b2-B), formula (II-b3-B) or formula (II-b4-B):

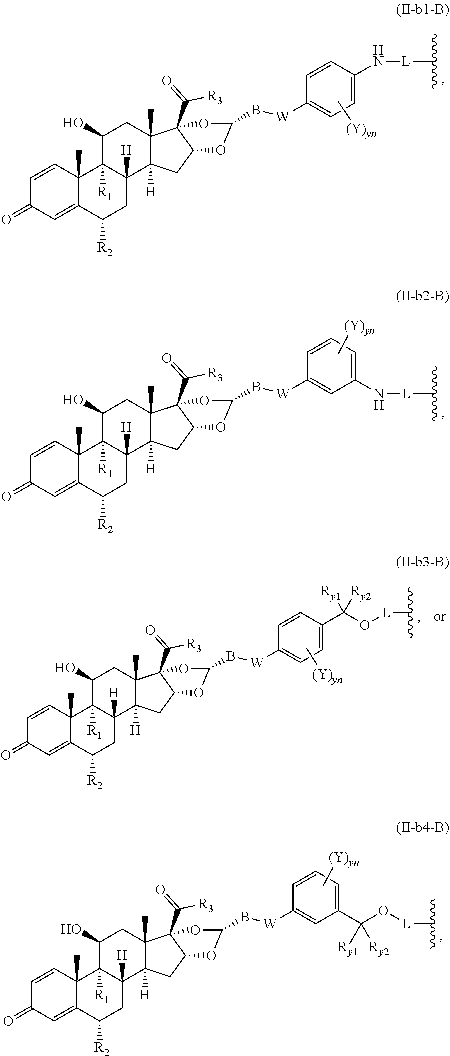

wherein
R₁, R₂, R₃, Ry₁, Ry₂ and Y may each independently be any group;
B may be selected from the group consisting of: optionally substituted

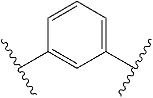 [SMILES: *c1cccc(*)c1]

and optionally substituted heteroarylene; for example, B may be selected from the group consisting of: optionally substituted

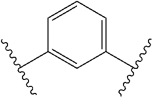 [SMILES: *c1cccc(*)c1]

and optionally substituted C₂-C₄ heteroarylene; W may be absent or W may be any group, wherein, when B may be optionally substituted

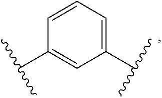 [SMILES: *c1cccc(*)c1]

W may not be —S—; yn may be a number equal to or greater than 0; L may be absent or may be any group.

For example, provided is a compound comprising a structure shown as formula (II-B), formula (II-a1-B), formula (II-a2-B), formula (II-b1-B), formula (II-b2-B), formula (II-b3-B) or formula (II-b4-B) or a tautomer, a mesomer, a racemate, an enantiomer or a diastereoisomer thereof, or a mixture thereof, or a pharmaceutically acceptable salt thereof, wherein L may comprise Tr.

The present application provides a compound of formula (II-C) or a tautomer, a mesomer, a racemate, an enantiomer or a diastereoisomer thereof, or a mixture thereof, or a pharmaceutically acceptable salt thereof,

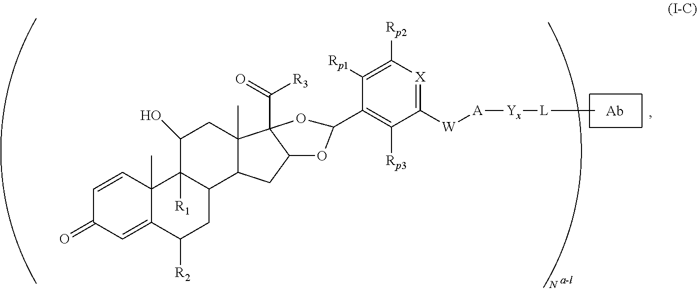

wherein
R₁, R₂, R₃, Rₚ₁, Rₚ₂, and Rₚ₃ may each independently be any group;
X may be selected from the group consisting of: N and optionally substituted CH;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; when X may be CH and W may be —S—, A may not be phenyl;
L may be absent or may be any group, Ab may be a ligand, and N^{a-1} may be a number equal to or greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-C), formula (I-a2-C), formula (I-a3-C) or formula (I-a4-C):

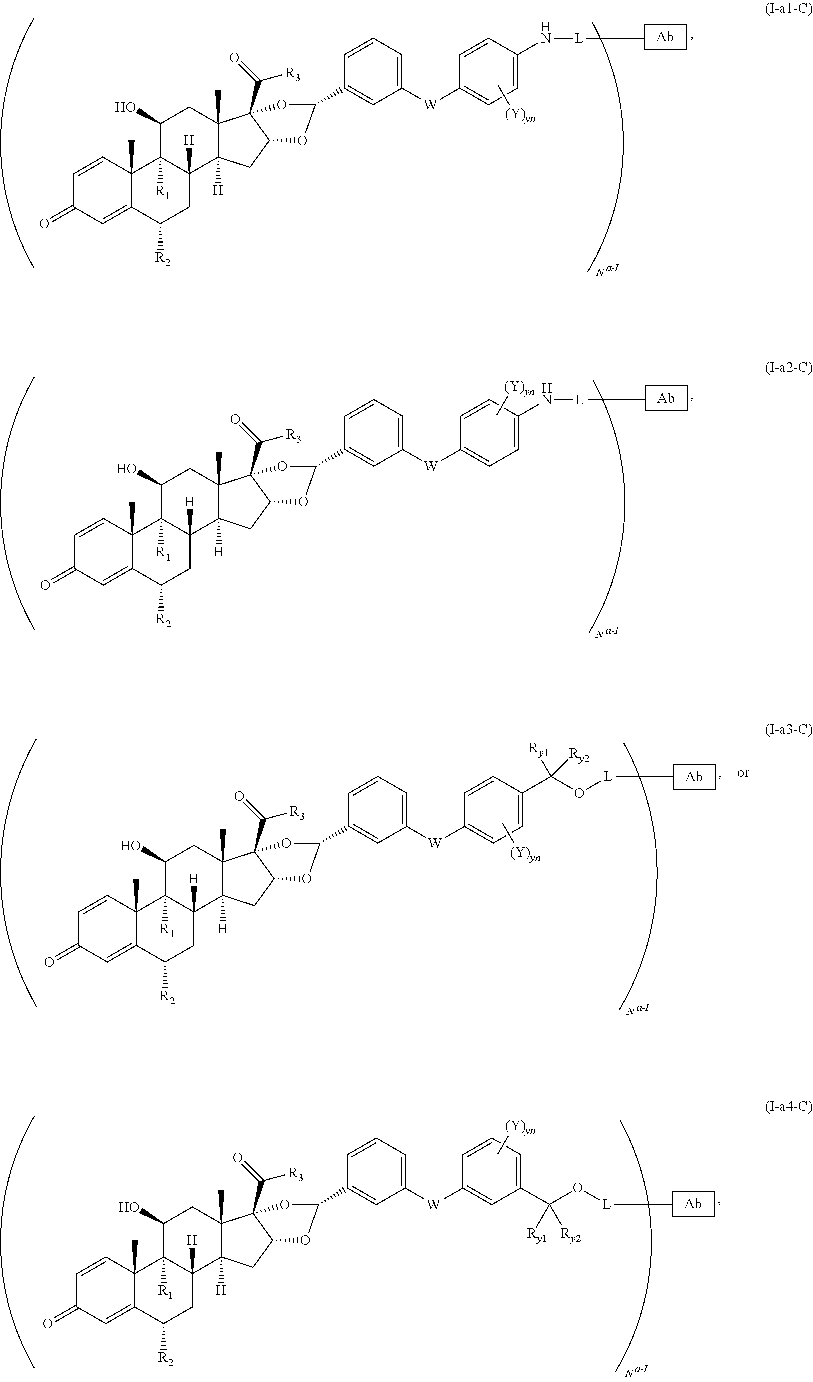

wherein
R₁, R₂, R₃, Ry₁, Ry₂ and Y may each independently be any group, yn may be a number equal to or greater than 0,
W may be absent or W may be any group, wherein W may not be —S—; L may be absent or may be any group; Ab may be a ligand; N^{a-1} may be a number equal to or greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-C):

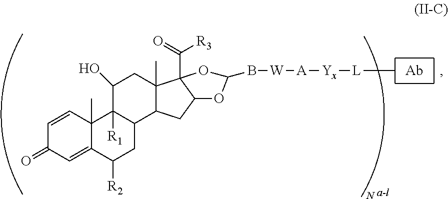

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; L may be absent or may be any group; Ab may be a ligand; N^{a-1} may be a number equal to or greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-C), formula (II-a2-C), formula (II-a3-C) or formula (II-a4-C):

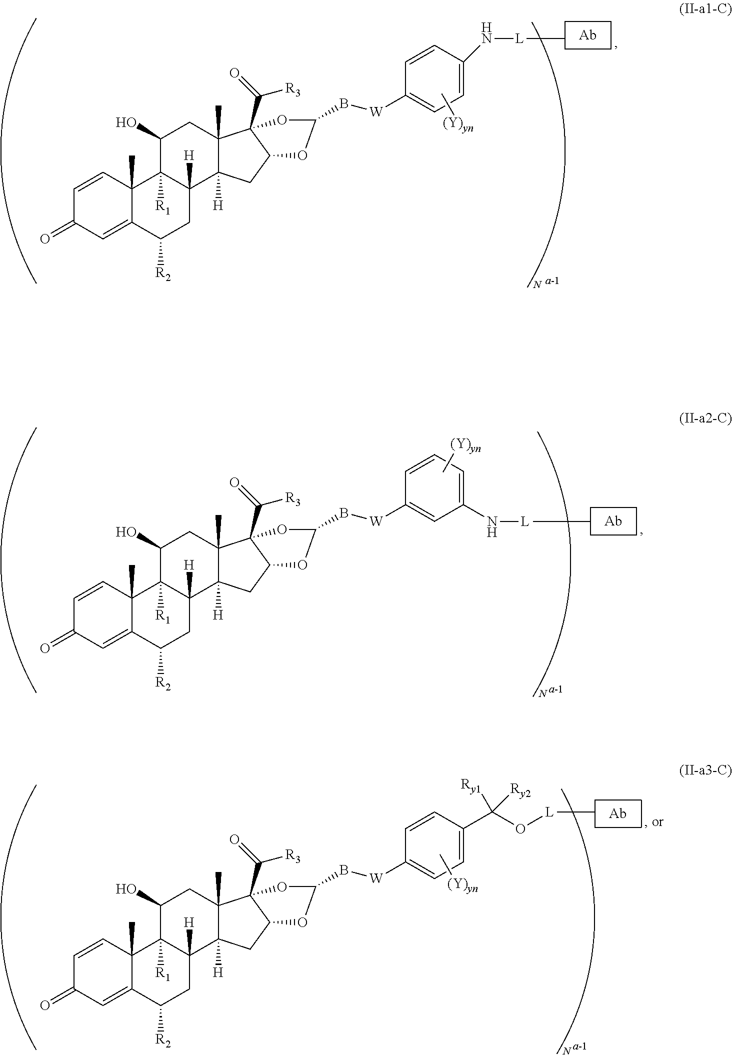

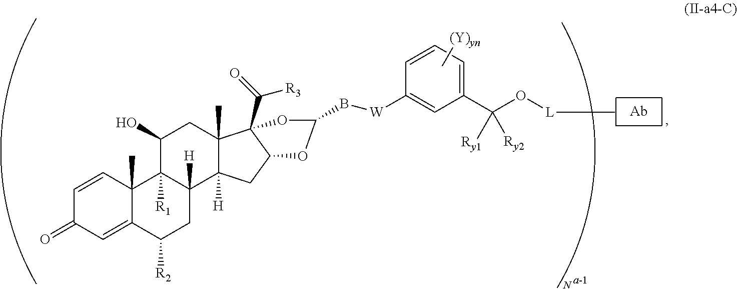

wherein
R₁, R₂, R₃, Ry₁, Ry₂, and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0; L may be absent or may be any group; Ab may be a ligand; N^{a-1} may be a number equal to or greater than 0.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-C), formula (II-b2-C), formula (II-b3-C) or formula (II-b4-C):

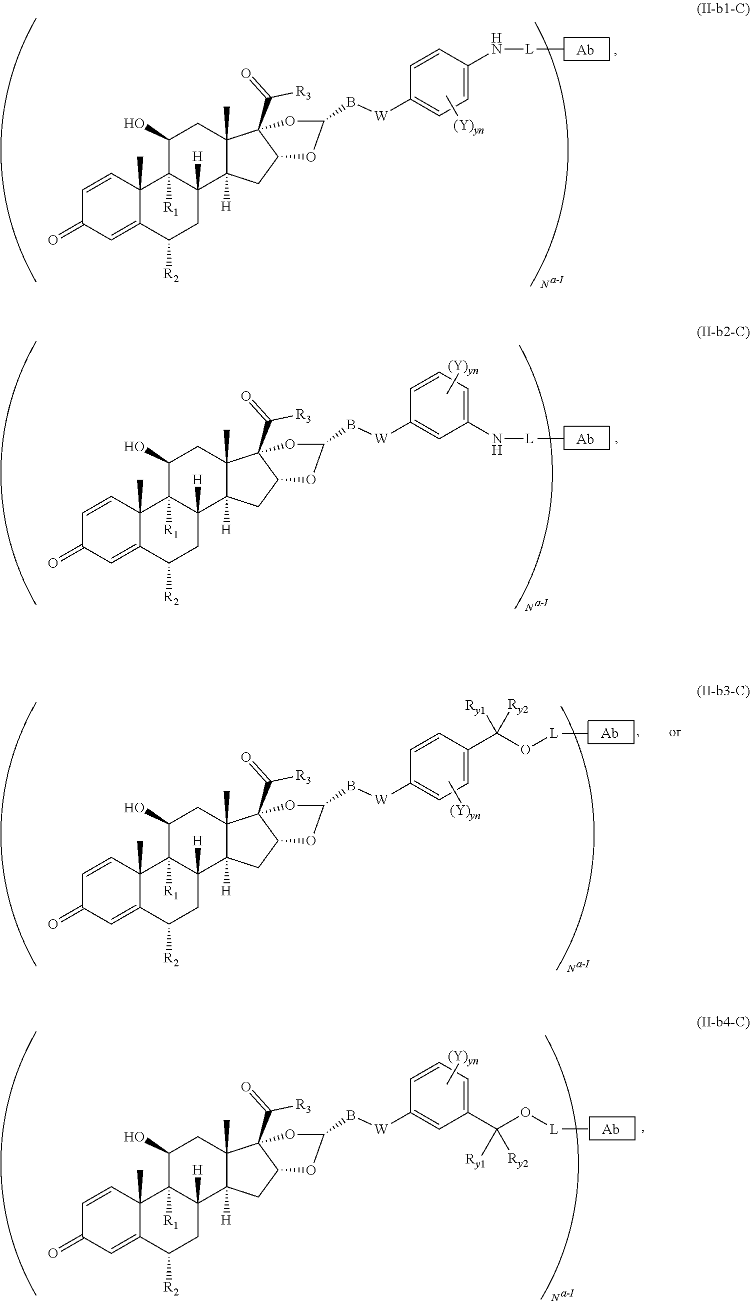

wherein R₁, R₂, R₃, Ry₁, Ry₂ and Y may each independently be any group;
B may be selected from the group consisting of: optionally substituted

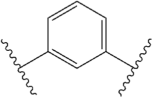 [SMILES: *c1cccc(*)c1]

and optionally substituted heteroarylene; for example, B may be selected from the group consisting of: optionally substituted

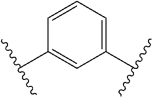 [SMILES: *c1cccc(*)c1]

and optionally substituted C₂-C₄ heteroarylene; W may be absent or W may be any group, wherein, when B may be optionally substituted

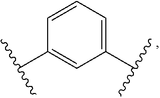 [SMILES: *c1cccc(*)c1]

W may not be —S—; yn may be a number equal to or greater than 0; L may be absent or may be any group; Ab may be a ligand; N^{a-1} may be a number equal to or greater than 0.

For example, provided is a compound comprising a structure shown as formula (II-C), formula (II-a1-C), formula (II-a2-C), formula (II-b1-C), formula (II-b2-C), formula (II-b3-C) or formula (II-b4-C) or a tautomer, a mesomer, a racemate, an enantiomer or a diastereoisomer thereof, or a mixture thereof, or a pharmaceutically acceptable salt thereof, wherein
L may comprise Tr.

The present application provides a compound of formula (II-D) or a tautomer, a mesomer, a racemate, an enantiomer or a diastereoisomer thereof, or a mixture thereof, or a pharmaceutically acceptable salt thereof,

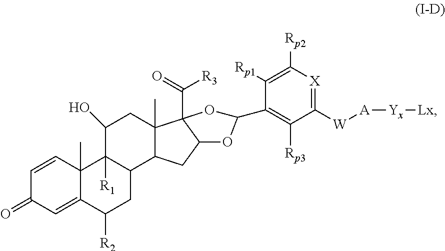

wherein
R₁, R₂, R₃, Rₚ₁, Rₚ₂, and Rₚ₃ may each independently be any group;
X may be selected from the group consisting of: N and optionally substituted CH;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; when X may be CH and W may be —S—, A may not be phenyl; Lx may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-D), formula (I-a2-D), formula (I-a3-D) or formula (I-a4-D):

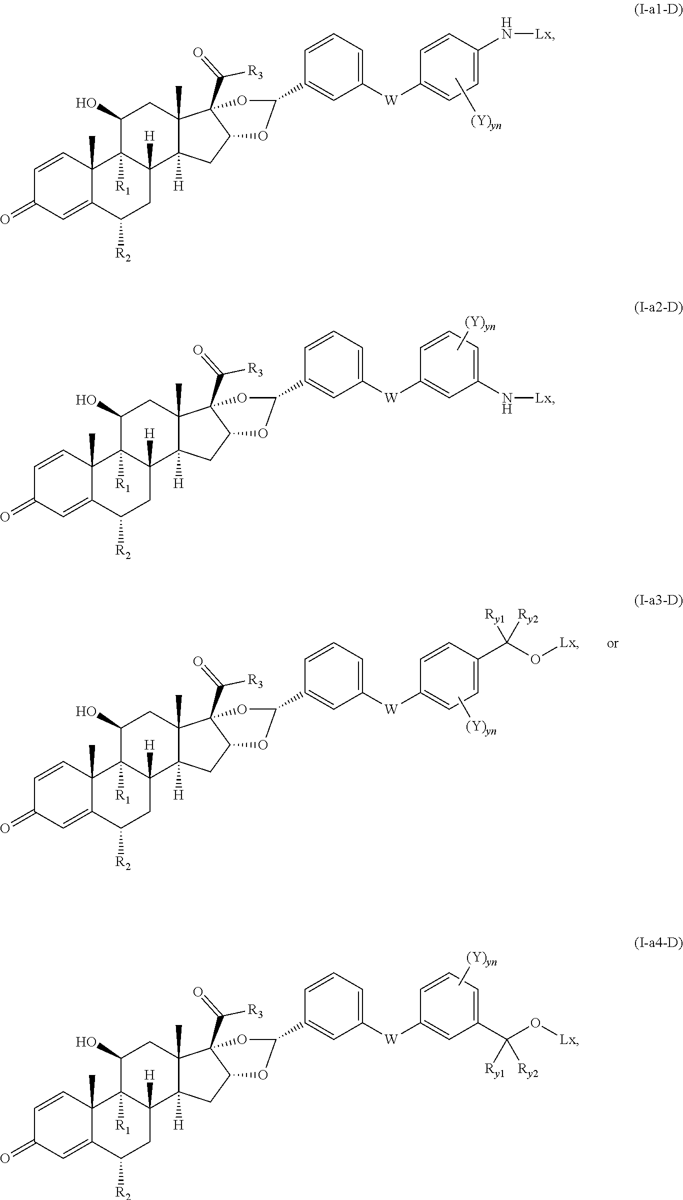

wherein
R₁, R₂, R₃, Ry₁, Ry₂ and Y may each independently be any group, yn may be a number equal to or greater than 0,
W may be absent or W may be any group, wherein W may not be —S—; Lx may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-D):

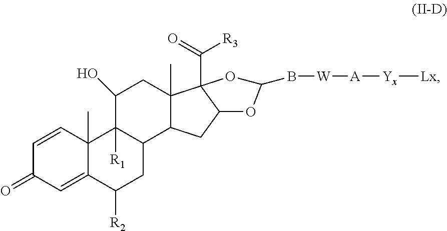

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; Lx may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-D), formula (II-a2-D), formula (II-a3-D) or formula (II-a4-D):

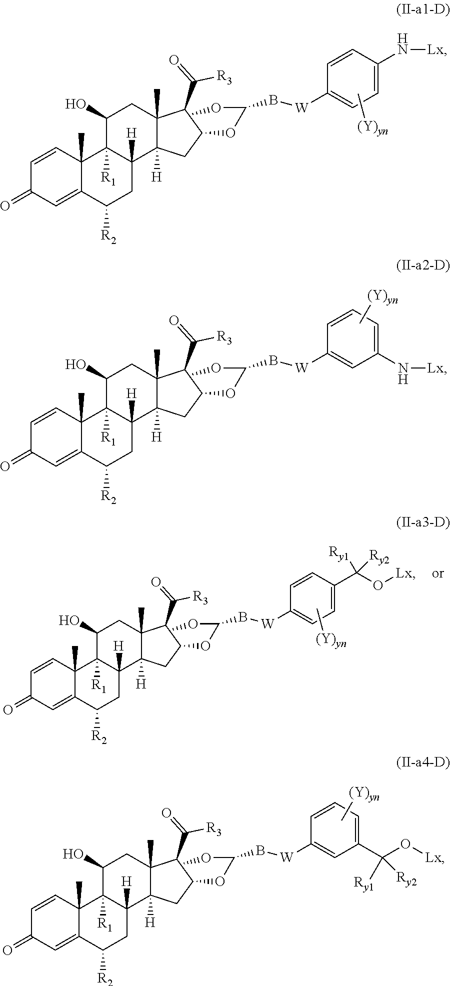

wherein
R₁, R₂, R₃, Ry₁, Ry₂, and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0; Lx may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-D), formula (II-b2-D), formula (II-b3-D) or formula (II-b4-D):

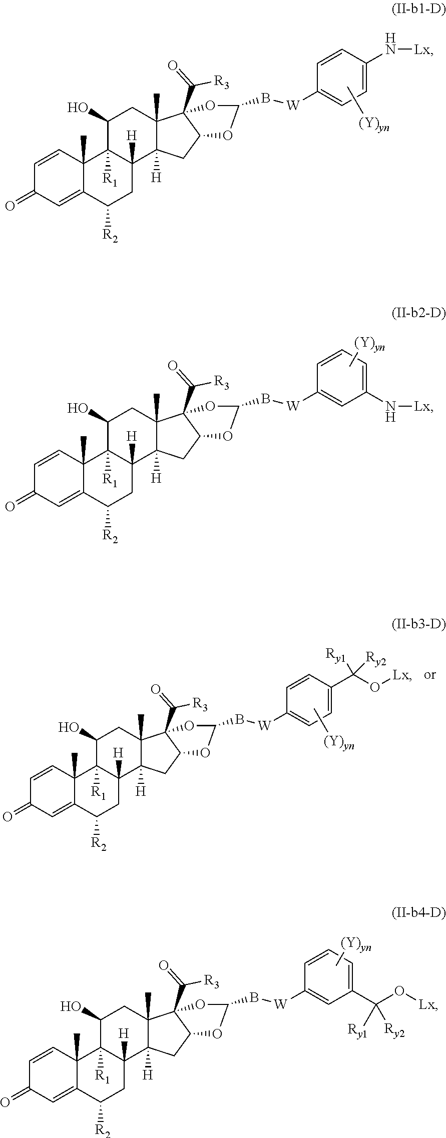

wherein
R₁, R₂, R₃, Ry₁, Ry₂ and Y may each independently be any group;
B may be selected from the group consisting of: optionally substituted

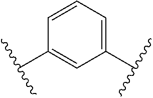 [SMILES: *c1cccc(*)c1]

and optionally substituted heteroarylene; for example, B may be selected from the group consisting of: optionally substituted

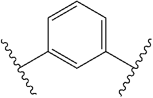 [SMILES: *c1cccc(*)c1]

and optionally substituted C₂-C₄ heteroarylene; W may be absent or W may be any group, wherein, when B may be optionally substituted

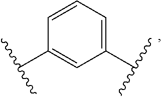 [SMILES: *c1cccc(*)c1]

W may not be —S—; yn may be a number equal to or greater than 0; Lx may be any group.

For example, provided is a compound comprising a structure shown as formula (II-D), formula (II-a1-D), formula (II-a2-D), formula (II-b1-D), formula (II-b2-D), formula (II-b3-D) or formula (II-b4-D) or a tautomer, a mesomer, a racemate, an enantiomer or a diastereoisomer thereof, or a mixture thereof, or a pharmaceutically acceptable salt thereof, wherein Lx comprises Tr.

The present application provides a compound of formula (II-E) or a tautomer, a mesomer, a racemate, an enantiomer or a diastereoisomer thereof, or a mixture thereof, or a pharmaceutically acceptable salt thereof,

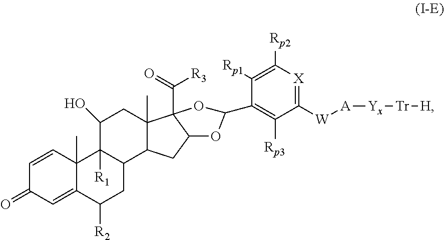

wherein
R₁, R₂, R₃, Rₚ₁, Rₚ₂, and Rₚ₃ may each independently be any group;
X may be selected from the group consisting of: N and optionally substituted CH;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; when X may be CH and W may be —S—, A may not be phenyl; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-E), formula (I-a2-E), formula (I-a3-E) or formula (I-a4-E):

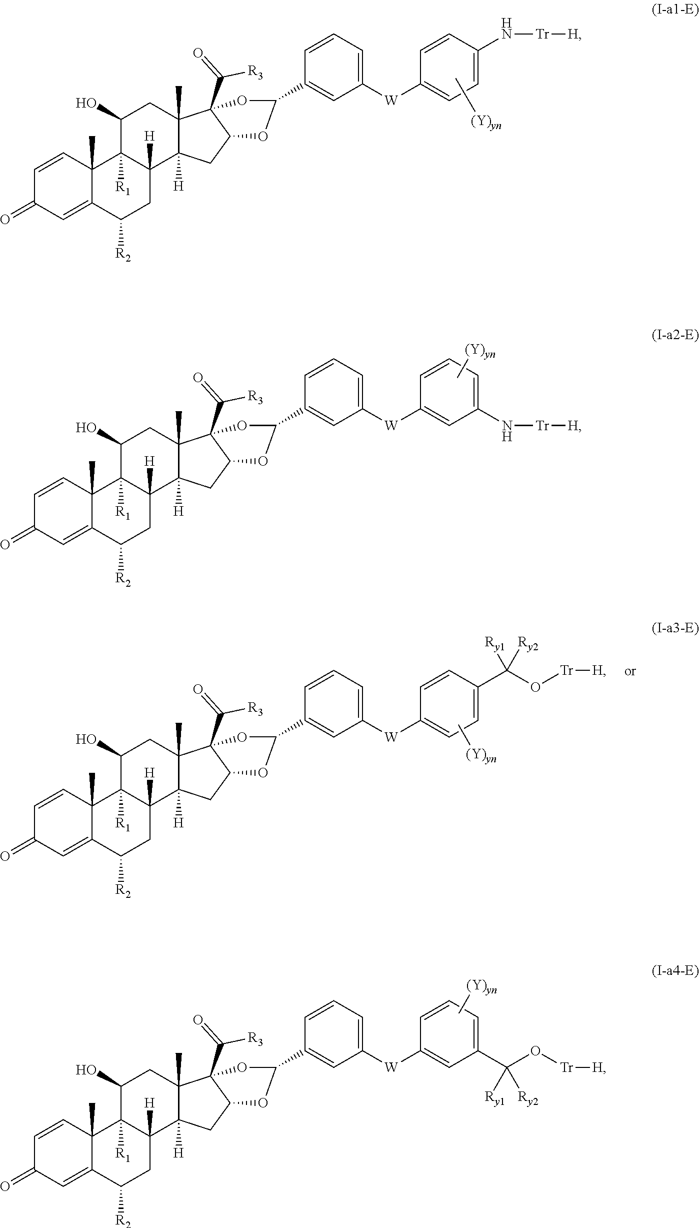

wherein
R₁, R₂, R₃, Ry₁, Ry₂ and Y may each independently be any group, yn may be a number equal to or greater than 0,
W may be absent or W may be any group, wherein W may not be —S—; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-E):

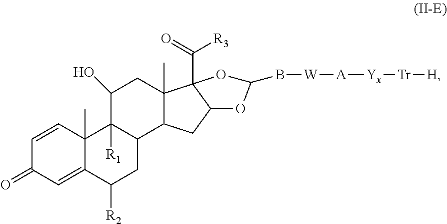

wherein
R₁, R₂ and R₃ may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene;
W may be absent or W may be any group;
A may be any group, wherein A may further be substituted with one or more Y, and Y is any group; Yₓ may be selected from the group consisting of: —O—, optionally substituted —CH₂—O—, —S—, optionally substituted —PH—, optionally substituted —P(═O)H—, and optionally substituted —NH—; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-E), formula (II-a2-E), formula (II-a3-E) or formula (II-a4-E):

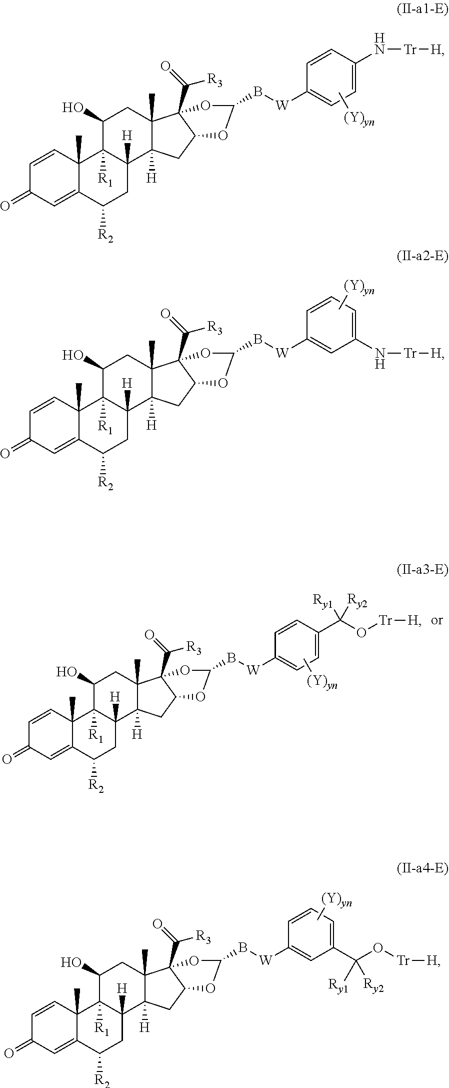

wherein
R₁, R₂, R₃, Ry₁, Ry₂, and Y may each independently be any group;
B may be selected from the group consisting of: optionally substituted alcylene, optionally substituted aliphatic heterocyclylene and optionally substituted heteroarylene; for example, B may be selected from the group consisting of: optionally substituted alcylene, optionally substituted aliphatic heterocyclylene and optionally substituted C₂-C₄ heteroarylene; W may be absent or W may be any group; yn may be a number equal to or greater than 0; Tr may be absent or may be any group.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-E), formula (II-b2-E), formula (II-b3-E) or formula (II-b4-E):

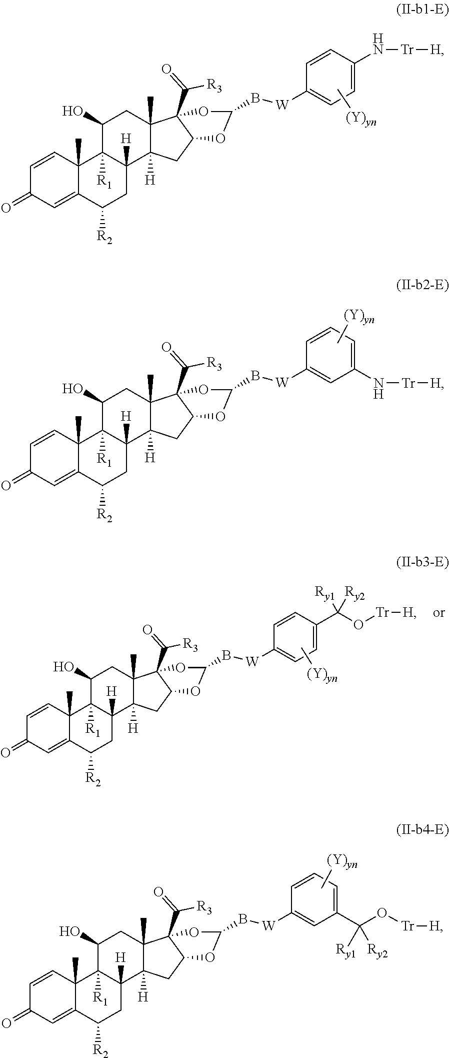

wherein
R₁, R₂, R₃, Ry₁, Ry₂, and Y may each independently be any group;
B may be selected from the group consisting of: optionally substituted

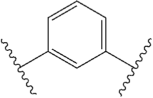 [SMILES: *c1cccc(*)c1]

and optionally substituted heteroarylene; for example, B may be selected from the group consisting of: optionally substituted

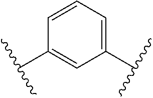 [SMILES: *c1cccc(*)c1]

and optionally substituted C₂-C₄ heteroarylene; W may be absent or W may be any group, wherein, when B may be optionally substituted

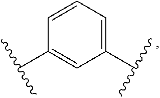 [SMILES: *c1cccc(*)c1]

W may not be —S—; yn may be a number equal to or greater than 0; Tr may be absent or may be any group.

For example, Ry₁ may be hydrogen, halogen, or optionally substituted alkyl. For example, Ry₁ may be hydrogen, halogen, or optionally substituted C₁-C₆ alkyl. For example, Ry₁ may be hydrogen, halogen, or optionally substituted C₁-C₃ alkyl. For example, Ry₁ may be hydrogen or optionally substituted methyl. For example, Ry₂ may be hydrogen, halogen, or optionally substituted alkyl. For example, Ry₂ may be hydrogen, halogen, or optionally substituted C₁-C₆ alkyl. For example, Ry₂ may be hydrogen, halogen, or optionally substituted C₁-C₃ alkyl. For example, Ry₂ may be hydrogen or optionally substituted methyl.

For example, Tr comprises —(SP¹)ₙ₁—, wherein
each SP¹ may independently be optionally substituted —CH₂—NH—, and
n1 may be a number equal to or greater than 0.

For example, in the compounds of the present application, n1 may be 1.

For example, in the compounds of the present application, SP¹ may be substituted with R¹, and each R¹ may be independently selected from the group consisting of: hydrogen and optionally substituted alkyl.

For example, in the compounds of the present application, each R¹ may be independently selected from the group consisting of: hydrogen and alkyl.

For example, in the compounds of the present application, each R¹ may be independently selected from the group consisting of: hydrogen and C₁-C₆ alkyl.

For example, in the compounds of the present application, each R¹ may be independently substituted with R₂, and each R₂ may be selected from the group consisting of: hydrogen and alkyl.

For example, in the compounds of the present application, R¹ may be substituted with R₂, and
each R² may be selected from the group consisting of hydrogen and C₁-C₆ alkyl.

For example, in the compounds of the present application, each SP¹ may be independently selected from the group consisting of: —CH₂—NH—, —CH(CH₃)—NH—, —C(CH₃)₂—NH—, —CH₂—N(CH₃)—, —CH(CH₃)—N(CH₃)— and —C(CH₃)₂—N(CH₃)—.

For example, in the compounds of the present application, each SP¹ may be independently selected from the group consisting of: —CH₂—NH—, —CH(CH₃)—NH—, —CH₂—N(CH₃)—, and —C(CH₃)—N(CH₃)—.

For example, in the compounds of the present application, Tr comprises —SP²—, wherein SP² may be optionally substituted —(CH₂)ₙ₂—,
wherein each methylene unit of SP² may be unreplaced; or at least 1 methylene unit of SP² may each independently be replaced by any group; wherein n2 may be at least 0.

For example, in the compounds of the present application, n2 may be selected from the group consisting of: 0, 1, 2 and 3.

For example, in the compounds of the present application, SP² may be substituted with R³, and each R₃ may be independently selected from the group consisting of hydrogen, ═O, optionally substituted —OH, optionally substituted —C(═O)H, and optionally substituted alkyl.

For example, in the compounds of the present application, each R₃ may be independently selected from the group consisting of hydrogen and alkyl.

For example, in the compounds of the present application, each R₃ may be independently selected from the group consisting of hydrogen and C₁-C₆ alkyl.

For example, in the compounds of the present application, R₃ may be substituted with R₄, and each R₄ may be independently selected from the group consisting of: hydrogen, ═O, optionally substituted —OH, optionally substituted —C(═O)H, and optionally substituted alkyl.

For example, in the compounds of the present application, each R₄ may be independently selected from the group consisting of hydrogen and alkyl.

For example, in the compounds of the present application, each R₄ may be independently selected from the group consisting of hydrogen and C₁-C₆ alkyl.

For example, in the compounds of the present application, at least 1 methylene unit of SP² may each independently be replaced by a group selected from the group consisting of optionally substituted —NHC(═O)—, optionally substituted —C(═O)NH—, —C(═O)—, —OC(═O)—, —C(═O)O—, optionally substituted —NH—, and —O—.

For example, in the compounds of the present application, at least 1, at least 2 or at least 3 methylene units of SP² may each independently be replaced by a group selected from the group consisting of: —C(═O)—, and optionally substituted —NH—.

For example, in the compounds of the present application, SP² may be selected from the group consisting of: optionally substituted —(CH₂)₂—, optionally substituted —(CH₂)₃—, optionally substituted —C(═O)—CH₂—NH—, optionally substituted —C(═O)—CH₂—N(CH₃)—, optionally substituted —(CH₂)₂—NH—, optionally substituted —(CH₂)₃—NH—, optionally substituted —(CH₂)₂—N(CH₃)— and optionally substituted —(CH₂)₃—N(CH₃)—.

For example, in the compounds of the present application, SP² may be a residue of an amino acid.

For example, in the compounds of the present application, SP² may be a residue of an amino acid that may be selected from the group consisting of: phenylalanine, isoleucine, leucine, tryptophan, valine, methionine, tyrosine, alanine, threonine, histidine, serine, glutamine, arginine, lysine, asparagine, glutamic acid, proline, citrulline, cysteine, aspartic acid, glycine, valine, alanine and phenylalanine.

For example, in the compounds of the present application, SP² may be a residue of an amino acid that may be selected from the group consisting of glutamic acid, lysine, citrulline, glycine, and alanine.

For example, in the compounds of the present application, SP² may be

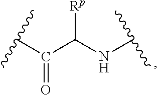

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

For example, in the compounds of the present application, SP² may be

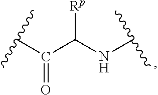

wherein R^{p} may be selected from the group consisting of: H, —CH₃, —(CH₂)₂—COOH, —(CH₂)₄—NH₂ and —(CH₂)₃—NHCONH₂.

For example, in the compounds of the present application, Tr comprises —(SP³)ₙ₃—, wherein each SP³ may be independently selected from the group consisting of: —O—, —S—, —C(═O)—, optionally substituted —NH—, —O—C(═O)—, —C(═O)—O—, optionally substituted —NH—C(═O)—, optionally substituted —NH—CH₂—, and optionally substituted —NH—CH₂—S—, and n3 may be a number equal to or greater than 0.

For example, in the compounds of the present application, n3 may be 1.

For example, in the compounds of the present application, SP³ may be substituted with R⁵, and each R⁵ may be independently selected from the group consisting of: hydrogen and alkyl.

For example, in the compounds of the present application, each R⁵ may be independently selected from the group consisting of: hydrogen and C₁-C₆ alkyl.

For example, in the compounds of the present application, SP³ may be selected from the group consisting of: —O—, —S—, —C(═O)—, optionally substituted —NH—, optionally substituted —N(CH₃)—, —C(═O)—O—, optionally substituted —C(═O)—NH—, optionally substituted —CH₂—NH—, optionally substituted —CH(CH₃)—NH—, optionally substituted —C(CH₃)₂—NH—, optionally substituted —CH₂—N(CH₃)—, optionally substituted —CH(CH₃)—N(CH₃)—, optionally substituted —C(CH₃)₂—N(CH₃)—, optionally substituted —S—CH₂—NH—, optionally substituted —S—CH₂—N(CH₃)—, optionally substituted —S—CH(CH₃)—NH— and optionally substituted —S—CH(CH₃)—N(CH₃)—.

For example, in the compounds of the present application, SP³ may be selected from the group consisting of: —O—, —S—, —C(═O)—, optionally substituted —NH—, —C(═O)—O—, —O—C(═O)—, optionally substituted —C(═O)—NH—, optionally substituted —CH₂—NH—, and optionally substituted —S—CH₂—NH—.

For example, in the compounds of the present application, SP³ may be —C(═O)—O—.

For example, in the compounds of the present application, Tr further comprises —SP⁴—, and SP⁴ may be

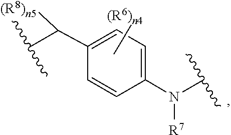

wherein each R⁶, each R⁷ and each R⁸ may each independently be absent, or each R⁶, each R⁷ and each R⁸ may each independently be selected from any group, and
n4 and n5 may each independently be a number that may be equal to or greater than 0.

For example, in the compounds of the present application, each R⁶, each R⁷ and each R⁸ may each independently be selected from the group consisting of hydrogen and alkyl.

For example, in the compounds of the present application, each R⁶, each R⁷ and each R⁸ may each independently be selected from the group consisting of hydrogen and C₁-C₆ alkyl.

For example, in the compounds of the present application, SP⁴ may be optionally substituted

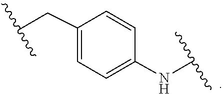 [SMILES: *Cc1ccc(N*)cc1]

For example, in the compounds of the present application, Tr may be absent.

For example, in the compounds of the present application, Tr comprises —(SP¹)ₙ₁—SP²—.

For example, in the compounds of the present application, Tr comprises —(SP¹)ₙ₁—SP²—(SP³)ₙ₃—.

For example, in the compounds of the present application, Tr comprises —(SP¹)ₙ₁—SP²—(SP³)ₙ₃—SP⁴—.

For example, in the compounds of the present application, Tr may be selected from the group consisting of:

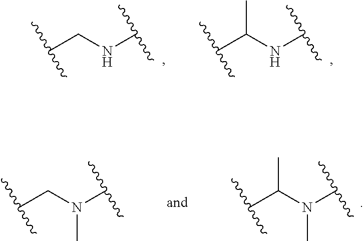

For example, in the compounds of the present application, Tr may be selected from the group consisting of:

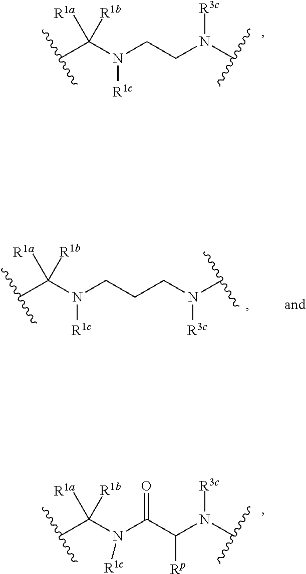

wherein, each R^{1a}, each R^{1b}, each R^{1c} and each R^{3c} may each independently be selected from the group consisting of hydrogen and C₁-C₆ alkyl, and
R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

For example, in the compounds of the present application, Tr may be selected from the group consisting of:

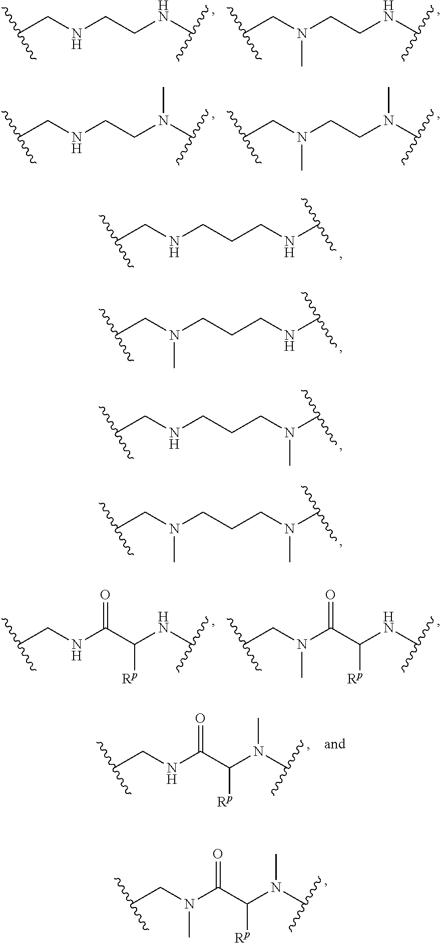

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —CsNH₆, —CH₂—C₆H₄—OH, —CH₂—COOH, —CH₂—CONH₂, —(CH₂)₂—COOH, —(CH₂)₄—NH₂, —(CH₂)₂—CONH2, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

For example, in the compounds of the present application, Tr may be selected from the group consisting of:

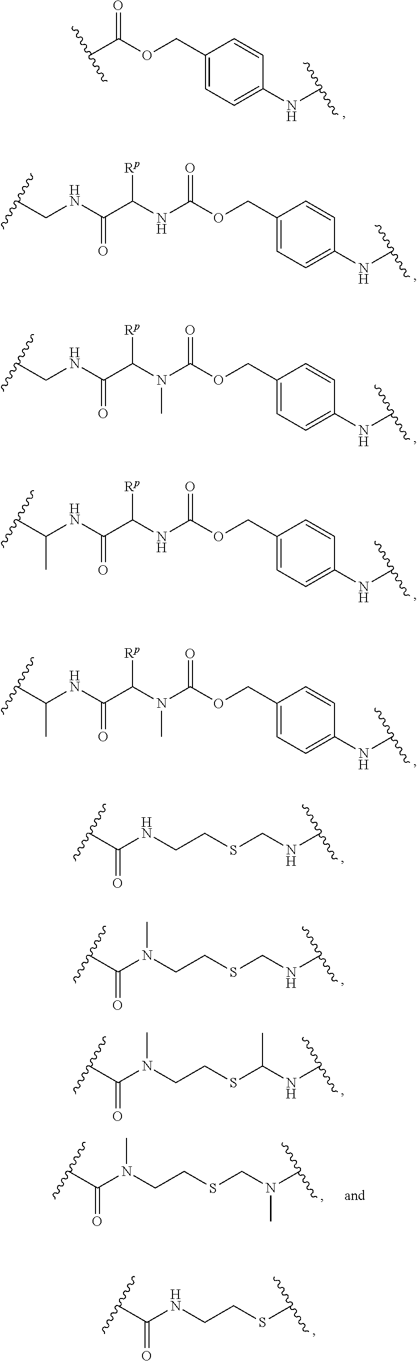

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —CsNH₆, —CH₂—C₆H₄—OH, —CH₂—COOH, —CH₂—CONH₂, —(CH₂)₂—COOH, —(CH₂)₄—NH₂, —(CH₂)₂—CONH2, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

For example, Lx comprises L^{1x}, and L^{1x} may be selected from the group consisting of: a group capable of coupling with amino, a group capable of coupling with sulfhydryl, and a click chemistry group.

For example, in the compounds of the present application, L^{1x} may be selected from the group consisting of:

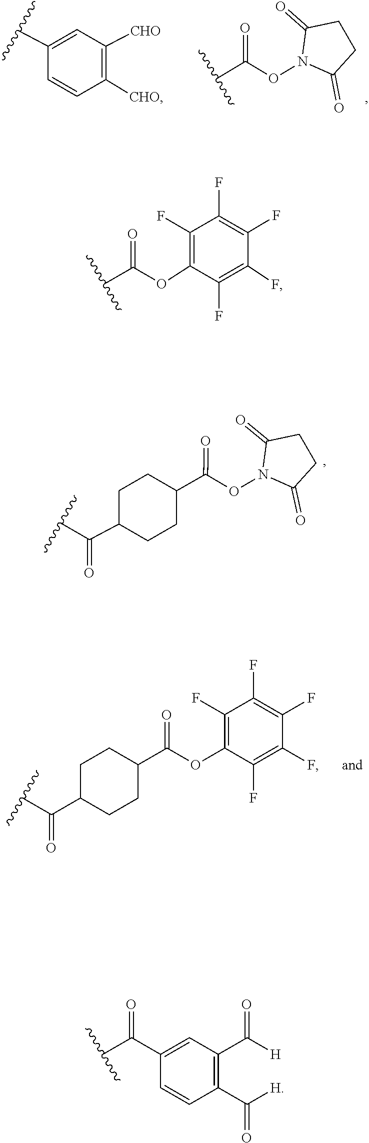

For example, in the compounds of the present application, L^{1x} may be selected from the group consisting of:

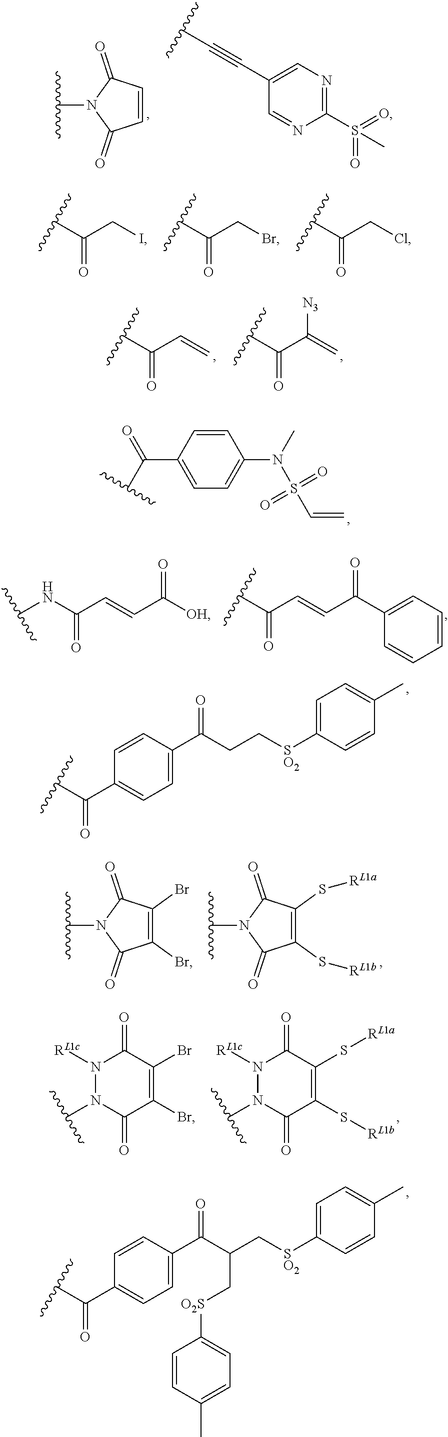

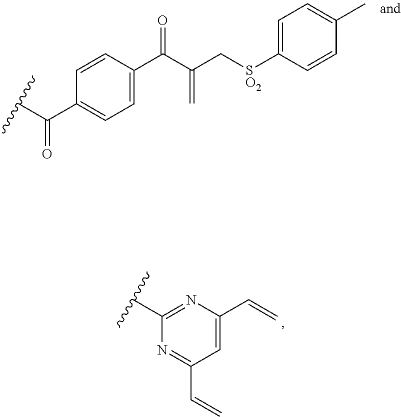

wherein each R^{L1a}, each R^{L1b} and each R^{L1c} may each independently be selected from any group.

For example, in the compounds of the present application, L^{1x} may be selected from the group consisting of:

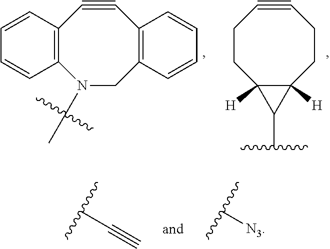

For example, in the compounds of the present application, L may comprise L¹, and L¹ may be any group.

For example, in the compounds of the present application, L¹ may be selected from the group consisting of: a divalent or trivalent residue formed by participation of amino in coupling, a divalent or trivalent residue formed by participation of sulfhydryl in coupling, and a divalent or trivalent residue formed by click chemistry coupling.

For example, L¹ may be selected from the group consisting of:

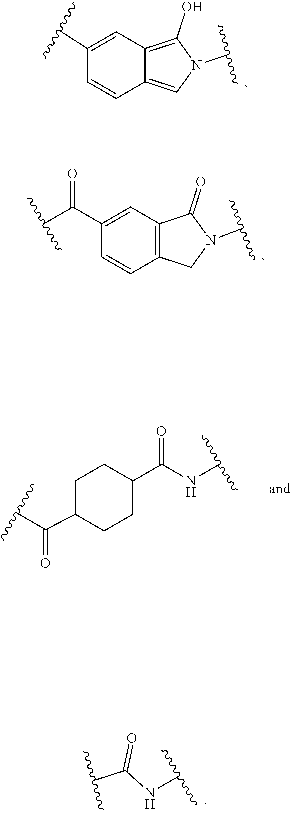 [SMILES: *c1ccc2cn(*)c(O)c2c1 ; *C(=O)c1ccc2c(c1)C(=O)N(*)C2 ; *NC(=O)C1CCC(C(*)=O)CC1 ; *NC(*)=O]

For example, L¹ may be selected from the group consisting of:

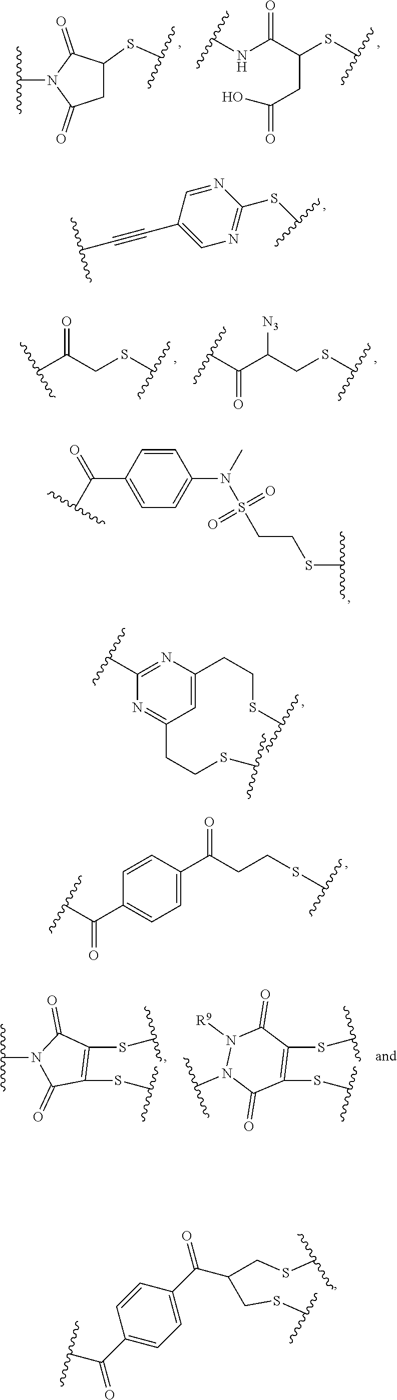

wherein R⁹ may be selected from any group.

For example, in the compounds of the present application, R⁹ may be selected from the group consisting of: hydrogen and alkyl.

For example, in the compounds of the present application, R⁹ may be selected from the group consisting of: hydrogen and C₁-C₆ alkyl.

For example, L¹ may be selected from the group consisting of:

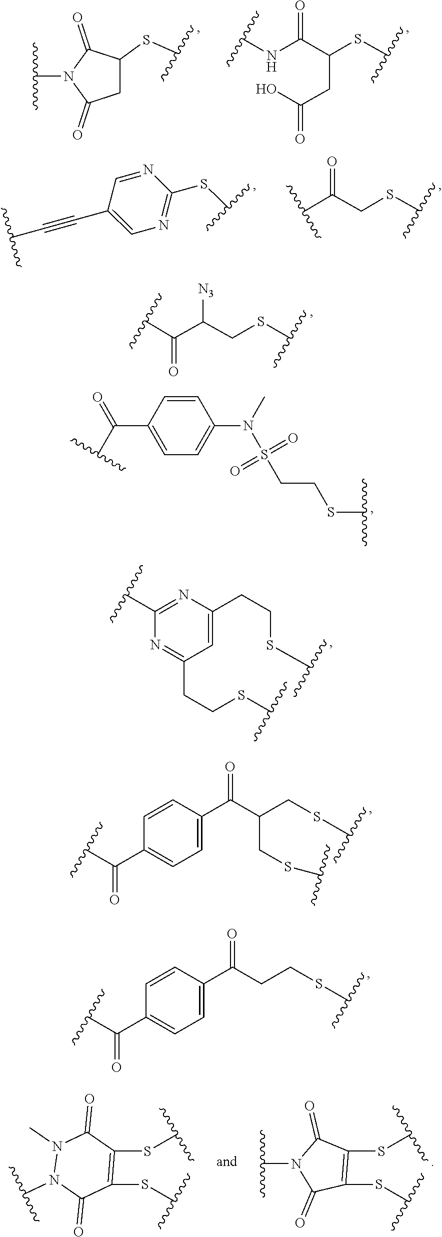

For example, L¹ may be selected from the group consisting of:

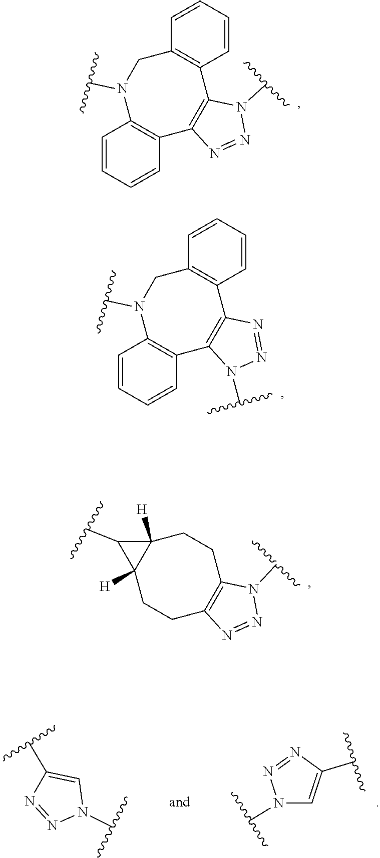 [SMILES: *N1Cc2ccccc2-c2c(nnn2*)-c2ccccc21 ; *N1Cc2ccccc2-c2nnn(*)c2-c2ccccc21 ; *C1[C@H]2CCc3nnn(*)c3CC[C@@H]12 ; *c1cn(*)nn1 ; *c1cn(*)nn1]

For example, in the compounds of the present application, L or Lx comprises L², and L² may be any group.

For example, in the compounds of the present application, L² comprises —X—, and X may be optionally substituted —(CH₂)ₚ₁—,
wherein each methylene unit of X may be unreplaced, or at least 1 methylene unit of X may each independently be replaced by a group selected from any group, and p1 may be a number equal to or greater than 0.

For example, in the compounds of the present application, p1 may be selected from the group consisting of: 0, 1, 2, 3, 4 and 5.

For example, in the compounds of the present application, X may be substituted with R¹⁰, and each R¹⁰ may be independently selected from the group consisting of: hydrogen, ═O, optionally substituted —OH, optionally substituted —C(O)H, and optionally substituted alkyl.

For example, in the compounds of the present application, each R¹⁰ may be independently selected from the group consisting of: hydrogen and alkyl.

For example, in the compounds of the present application, each R¹⁰ may be independently selected from the group consisting of: hydrogen and C₁-C₆ alkyl.

For example, in the compounds of the present application, R¹⁰ may be substituted with R¹¹, and
each R¹¹ may be independently selected from the group consisting of hydrogen, ═O, optionally substituted —OH, optionally substituted —C(O)H, and optionally substituted alkyl.

For example, in the compounds of the present application, each R¹¹ may be independently selected from the group consisting of hydrogen and alkyl.

For example, in the compounds of the present application, each R¹¹ may be independently selected from the group consisting of: hydrogen and C₁-C₆ alkyl.

For example, in the compounds of the present application, at least 1 methylene unit of X may each independently be replaced by a group selected from the group consisting of optionally substituted —NHC(O)—, optionally substituted —C(O)NH—, —C(O)—, —OC(O)—, —C(O)O—, optionally substituted —NH—, —S—, and —O—.

For example, in the compounds of the present application, 1 or 2 methylene units of X may each independently be replaced by a group selected from the group consisting of optionally substituted —C(O)NH—, —S—, —C(O)—, —OC(O)—, —C(O)O—, and optionally substituted —NH—.

For example, in the compounds of the present application, X may be selected from the group consisting of: —C(O)—, —OC(O)—, —C(O)O—, —NR^{x}— and —S—C(R^{xa})(R^{xb})—CH₂—NH—C(O)—, wherein
each R^{x}, each R^{xa} and each R^{xb} may each independently be selected from the group consisting of: hydrogen and alkyl.

For example, in the compounds of the present application, X may be selected from the group consisting of —C(O)— and —S—C(R^{xa})(R^{xb})CH₂—NH—C(O)—.

For example, in the compounds of the present application, L² further comprises -LB-, and
LB may be

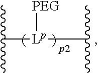

wherein L^{p} may be a trivalent residue, PEG may comprise a polyethylene glycol unit, and p2 may be a number equal to or greater than 0.

For example, in the compounds of the present application, p2 may be selected from the group consisting of: 0, 1, 2, 3, 4 and 5.

For example, L^{p} may be selected from the group consisting of amino acid, amino alcohol, amino aldehyde and polyamine.

For example, L^{p} may be selected from the group consisting of aspartic acid, glutamic acid, histidine, lysine, arginine, serine, cysteine, threonine, and tyrosine.

For example, L^{p} may be selected from the group consisting of: aspartic acid, glutamic acid, and lysine.

For example, L^{p} may be:

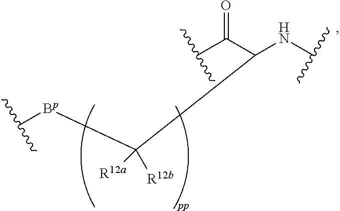

wherein each R^{12a} and each R^{12b} may each independently be absent, or each R^{12a} and R^{12b} may each independently be selected from any group;
B^{p} may be selected from the group consisting of —NH—, —N(CH₃)—, —C(O)— and —O—;
pp may be a number equal to or greater than 0.

For example, in the compounds of the present application, pp may be selected from the group consisting of: 0, 1, 2, 3 and 4.

For example, in the compounds of the present application, each R^{12a} and each R^{12b} may each independently be selected from the group consisting of hydrogen and alkyl.

For example, in the compounds of the present application, each R^{12a} and each R^{12b} may each independently be selected from the group consisting of hydrogen and C₁-C₆ alkyl.

For example, L^{p} may be selected from the group consisting of:

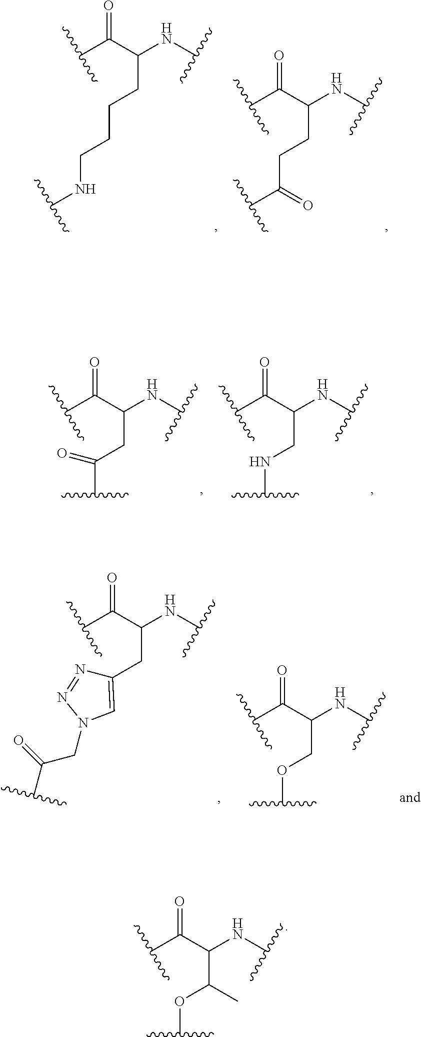

For example, in the compounds of the present application, PEG may comprise —(PX—(CH₂CH₂O)ₚ₋₃)ₚ₋₄—, wherein p-3 and p-4 may each independently be at least 1,
PX may comprise optionally substituted —(CH₂)ₚ₋₅—, and
each methylene unit of PX may be unsubstituted, or at least 1 methylene unit of PX may each independently be replaced by any group,
wherein p-5 may be at least 0.

For example, in the compounds of the present application, p-5 may be selected from the group consisting of: 0, 1, 2, 3, 4 and 5.

For example, PX may be substituted with R¹³, and each R¹³ may be independently selected from the group consisting of: hydrogen, ═O, optionally substituted —OH, optionally substituted —C(O)H, and optionally substituted alkyl.

For example, in the compounds of the present application, each R¹³ may be independently selected from the group consisting of hydrogen and alkyl.

For example, in the compounds of the present application, each R¹³ may be independently selected from the group consisting of: hydrogen and C₁-C₆ alkyl.

For example, R¹³ may be substituted with R¹⁴, and each R¹⁴ may be independently selected from the group consisting of: hydrogen, ═O, optionally substituted —OH, optionally substituted —C(O)H, and optionally substituted alkyl.

For example, in the compounds of the present application, each R¹⁴ may be independently selected from the group consisting of hydrogen and alkyl.

For example, in the compounds of the present application, each R¹⁴ may be independently selected from the group consisting of: hydrogen and C₁-C₆ alkyl.

For example, in the compounds of the present application, at least 1 methylene unit of PX may each independently be replaced by a group selected from the group consisting of optionally substituted —NHC(O)—, optionally substituted —C(O)NH—, —C(O)—, —OC(O)—, —C(O)O—, optionally substituted —NH—, and —O—.

For example, in the compounds of the present application, 1 or 2 methylene units of PX may each independently be replaced by a group selected from the group consisting of: —C(O)—, —OC(O)—, —C(O)O—, and optionally substituted —NH—.

For example, in the compounds of the present application, PX may be selected from the group consisting of —C(O)— and optionally substituted —NH—.

For example, in the compounds of the present application, PX may be selected from the group consisting of —C(O)— and —NH—.

For example, in the compounds of the present application, p-3 may be selected from the group consisting of 1, 2, 3, 4, 5, 6, 7, 8, 9, 10, 11, 12, 13, 14, 15, 16, 17, 18, 19, 20, 21, 22, 23 and 24.

For example, in the compounds of the present application, p-3 may be selected from the group consisting of: 4. 6, 8, 10, 12 and 24.

For example, in the compounds of the present application, p-3 may be selected from the group consisting of: 8. 9, 10, 12 and 24.

For example, in the compounds of the present application, p-4 may be selected from the group consisting of: 1, 2, 3, 4 and 5.

For example, in the compounds of the present application, PEG further comprises —PZ, and PZ may be absent or PZ may be selected from any group.

For example, in the compounds of the present application, PZ may be selected from the group consisting of hydrogen and optionally substituted alkyl.

For example, in the compounds of the present application, PZ may be selected from the group consisting of hydrogen and optionally substituted C₁-C₆ alkyl.

For example, in the compounds of the present application, PZ may be substituted with R¹⁵, and R¹⁵ may be selected from the group consisting of: —OH, —C(O)H, —NH₂, and —C(═O)OH.

For example, in the compounds of the present application, PZ may be selected from the group consisting of hydrogen, —CH₂—CH₂—C(O)OH and methyl.

For example, in the compounds of the present application, PEG may be selected from the group consisting of:

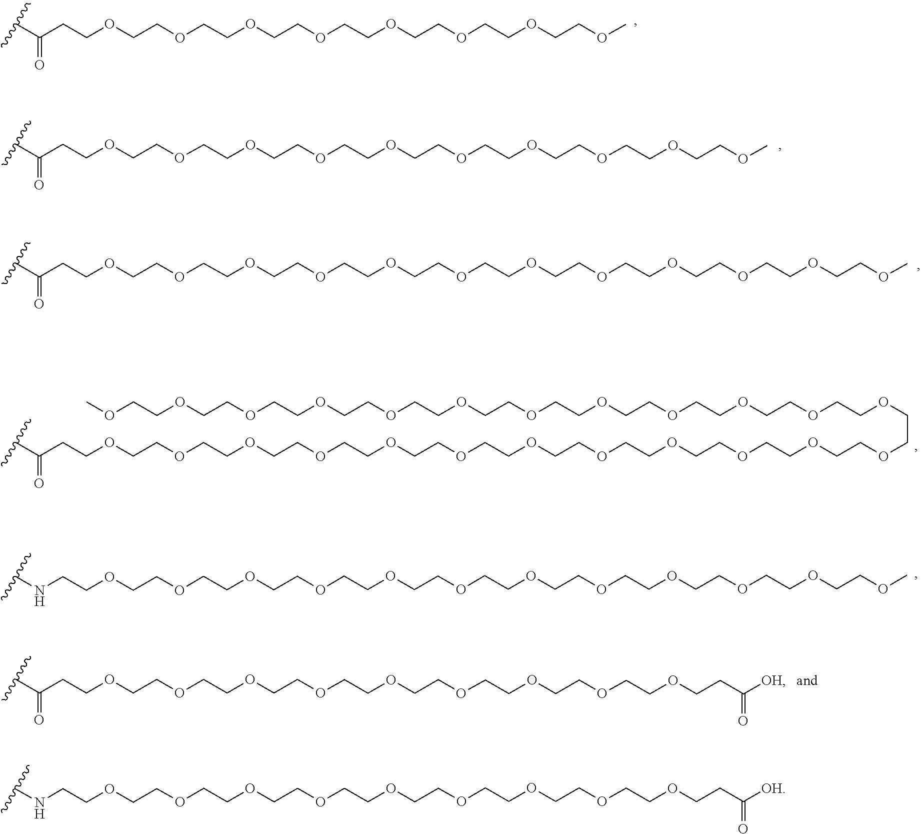

For example, in the compounds of the present application, LB may be selected from the group

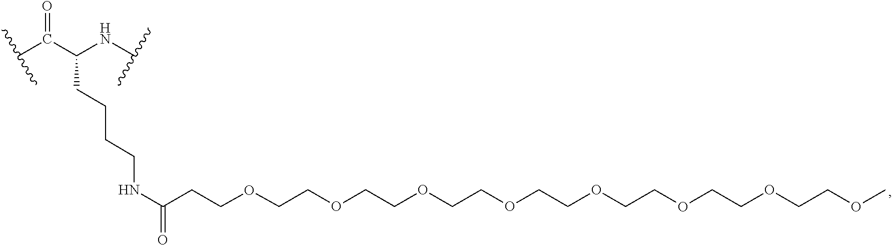 [SMILES: *N[C@H](CCCCNC(=O)CCOCCOCCOCCOCCOCCOCCOCCOC)C(*)=O]

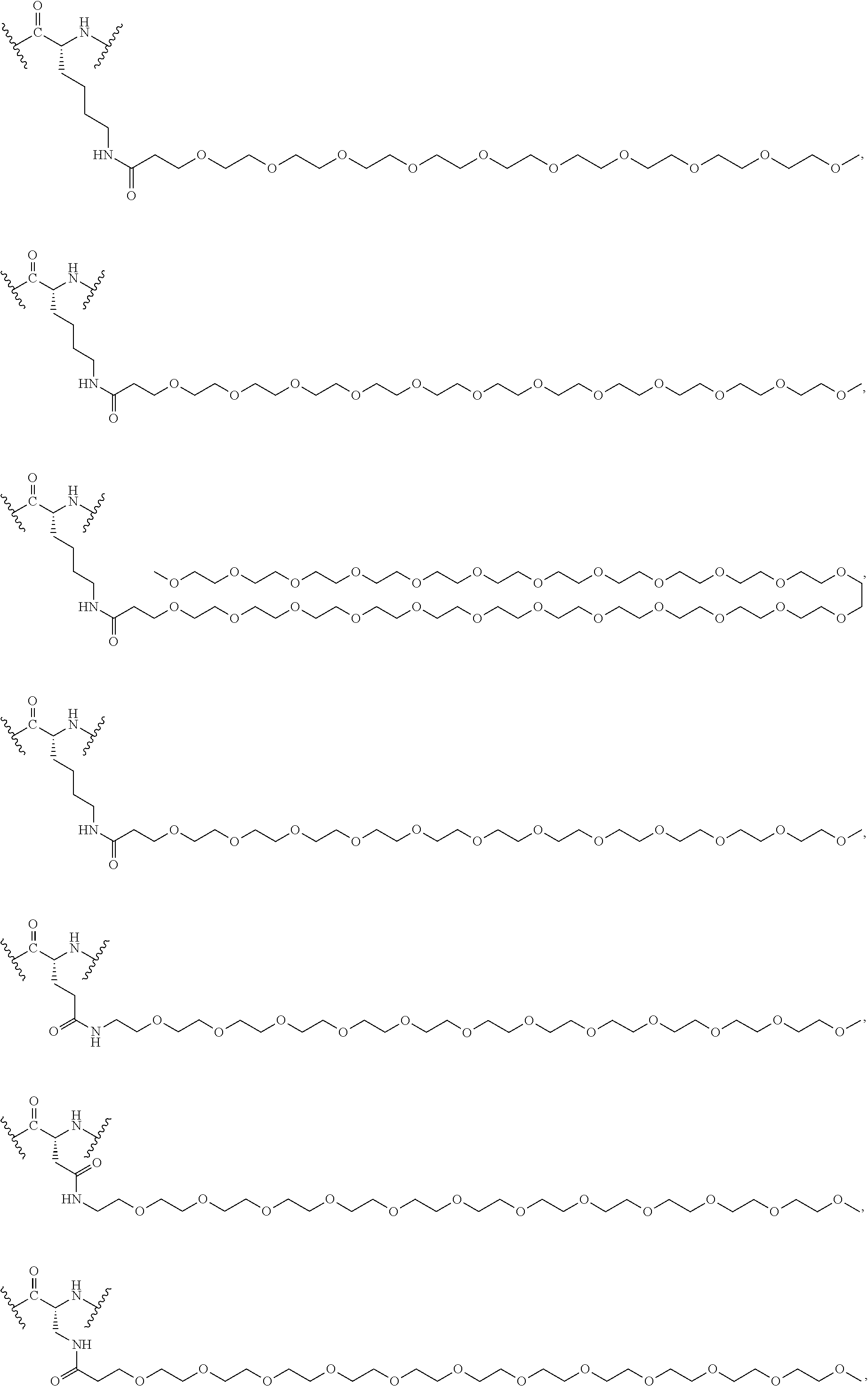

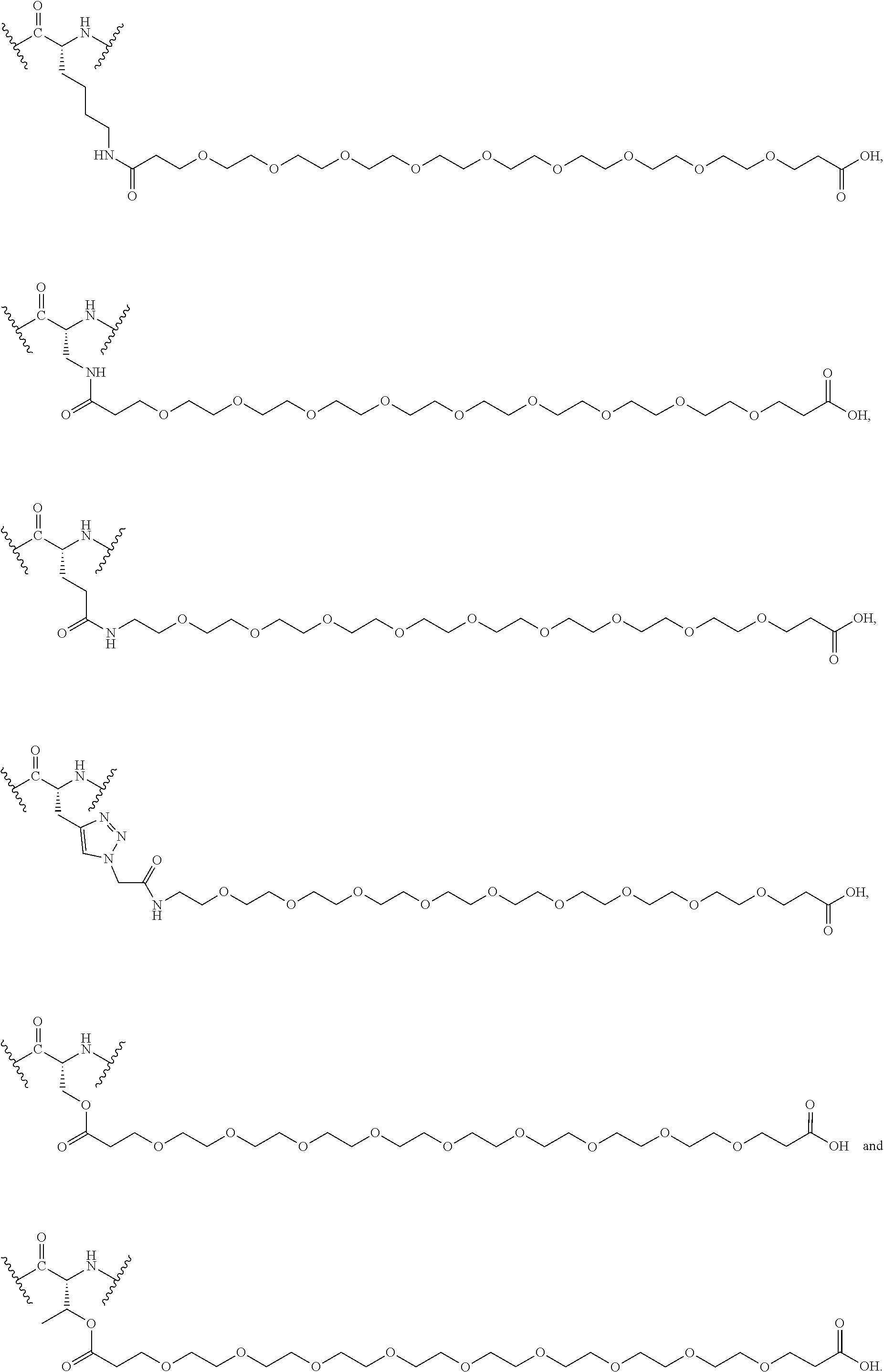 [SMILES: *N[C@H](CCCCNC(=O)CCOCCOCCOCCOCCOCCOCCOCCOCCOCCC(=O)O)C(*)=O ; *N[C@H](CNC(=O)CCOCCOCCOCCOCCOCCOCCOCCOCCOCCC(=O)O)C(*)=O ; *N[C@H](CCC(=O)NCCOCCOCCOCCOCCOCCOCCOCCOCCOCCC(=O)O)C(*)=O ; *N[C@H](Cc1cn(CC(=O)NCCOCCOCCOCCOCCOCCOCCOCCOCCOCCC(=O)O)nn1)C(*)=O ; *N[C@H](COC(=O)CCOCCOCCOCCOCCOCCOCCOCCOCCOCCC(=O)O)C(*)=O ; *N[C@@H](C(*)=O)C(C)OC(=O)CCOCCOCCOCCOCCOCCOCCOCCOCCOCCC(=O)O]

For example, in the compounds of the present application, L² further comprises -LY-, and LY may be —Oₚ₋₇—(CH₂CH₂O)ₚ₋₆—, wherein p-6 and p-7 may each independently be at least 0.

For example, in the compounds of the present application, p-7 may be selected from the group consisting of: 0 and 1.

For example, in the compounds of the present application, p-6 may be selected from the group consisting of: 0, 1, 2, 3, 4, 5, 6, 7, 8, 9, 10, 11 and 12.

For example, in the compounds of the present application, p-6 may be selected from the group consisting of: 3, 4, 5, 6, 8, 10 and 12.

For example, in the compounds of the present application, p-6 may be selected from the group consisting of: 3, 4, 5, 6, 7 and 8.

For example, in the compounds of the present application, p-6 may be selected from the group consisting of: 3, 5 and 7.

For example, in the compounds of the present application, LY may be selected from the group consisting of: —(CH₂CH₂O)₃—, —(CH₂CH₂O)₄—, —(CH₂CH₂O)₅—, —(CH₂CH₂O)₆—, —(CH₂CH₂O)₇— and —(CH₂CH₂O)₈—.

For example, in the compounds of the present application, LY may be selected from the group consisting of: —(CH₂CH₂O)₃—, —(CH₂CH₂O)₅— and —(CH₂CH₂O)₇—.

For example, in the compounds of the present application, L² further comprises —Z—, and Z may be optionally substituted —(CH₂)ₚ₋₈—, wherein each methylene unit of Z may be unreplaced, or at least 1 methylene unit of Z may each independently be replaced by a group selected from any group, and p-8 may be at least 0.

For example, in the compounds of the present application, p-8 may be selected from the group consisting of: 0, 1, 2, 3, 4, 5, 6 and 7.

For example, in the compounds of the present application, Z may be substituted with R¹⁶, and each R¹⁶ may be independently selected from the group consisting of: hydrogen, ═O, optionally substituted —OH, optionally substituted —C(O)H, and optionally substituted alkyl.

For example, in the compounds of the present application, each R¹⁶ may be independently selected from the group consisting of: hydrogen and alkyl.

For example, in the compounds of the present application, each R¹⁶ may be independently selected from the group consisting of: hydrogen and C₁-C₆ alkyl.

For example, in the compounds of the present application, R¹⁶ may be substituted with R¹⁷, and each R¹⁷ may be independently selected from the group consisting of: hydrogen and alkyl.

For example, in the compounds of the present application, each R¹⁷ may be independently selected from the group consisting of: hydrogen and C₁-C₆ alkyl.

For example, in the compounds of the present application, at least 1 methylene unit of Z may each independently be replaced by a group selected from the group consisting of: optionally substituted —NHC(O)—, optionally substituted —C(O)NH—, —C(O)—, —OC(O)—, —C(O)O—, optionally substituted —NH—, and —O—.

For example, in the compounds of the present application, 1 or 2 methylene units of Z may each independently be replaced by a group selected from the group consisting of: optionally substituted —C(O)NH—, optionally substituted —NHC(O)—, —C(O)—, —OC(O)—, —C(O)O—, and optionally substituted —NH—.

For example, in the compounds of the present application, Z may be selected from the group consisting of: optionally substituted —NH—, optionally substituted —(CH₂)₂—, optionally substituted —(CH₂)₅—, optionally substituted —C(O)—(CH₂)₂—, optionally substituted —C(O)—(CH₂)₄—, optionally substituted —C(O)—(CH₂)₅—, optionally substituted —(CH₂)₂—NH—C(O)—(CH₂)₂—, optionally substituted —(CH₂)₂—NH—C(O)—(CH₂)₂—C(O)—, optionally substituted —(CH₂)₂—NH— and optionally substituted —(CH₂)₂—NH—C(O)—O—CH₂—.

For example, in the compounds of the present application, L² may be absent.

For example, in the compounds of the present application, L² comprises —X—Z—, —Z— or —X—.

For example, in the compounds of the present application, L² comprises —X—Z—.

For example, in the compounds of the present application, L² may be selected from the group consisting of:

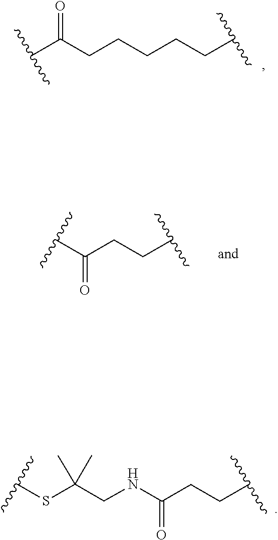 [SMILES: *CCCCCC(*)=O ; *CCC(*)=O ; *CCC(=O)NCC(C)(C)S*]

For example, in the compounds of the present application, L² comprises —X-LY-Z—, -LY-Z—, —X-LY- or -LY-.

For example, in the compounds of the present application, L² comprises —X-LY-Z—.

For example, in the compounds of the present application, L² may be selected from the group consisting of:

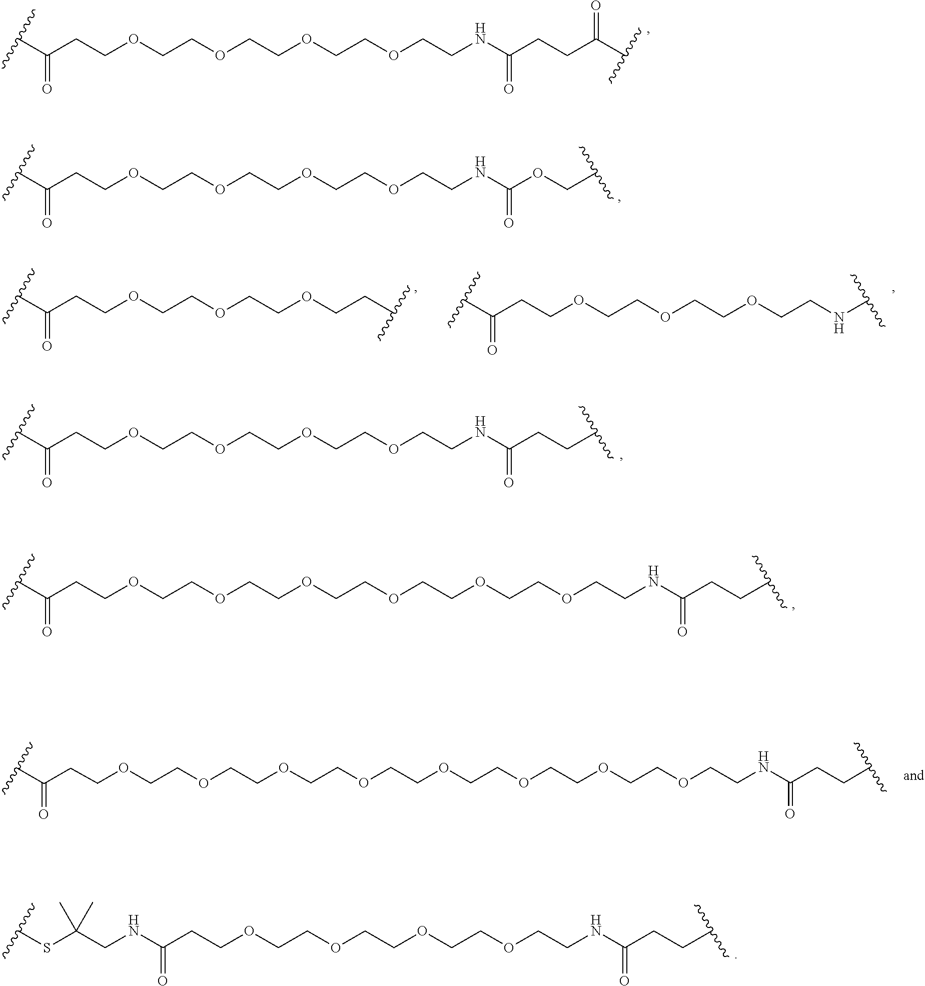

For example, in the compounds of the present application, L² comprises —X-LB-Z—, -LB-Z—, —X-LB- or -LB-.

For example, in the compounds of the present application, L² comprises -LB-Z— or -LB-.

For example, in the compounds of the present application, L² may be selected from the group

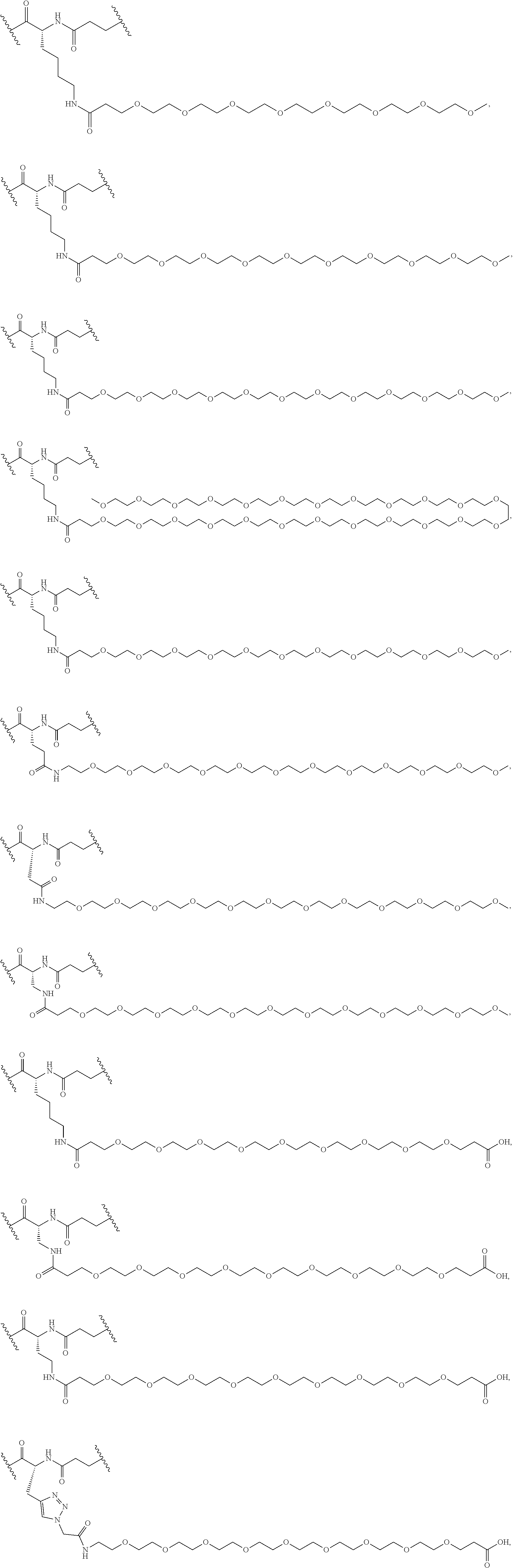

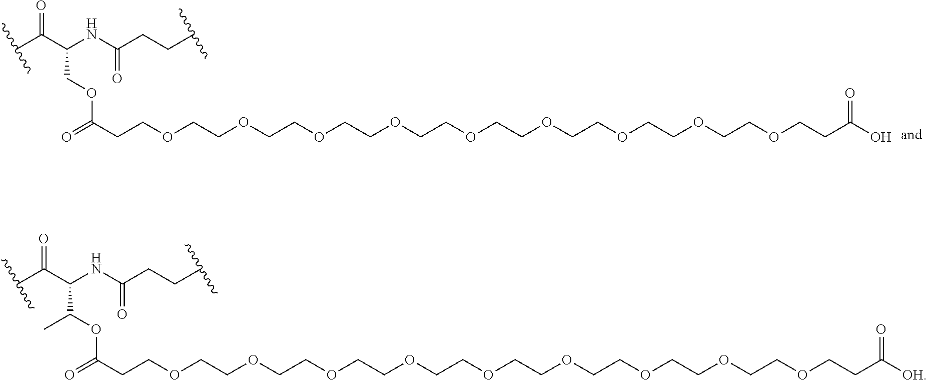 [SMILES: *CCC(=O)N[C@H](COC(=O)CCOCCOCCOCCOCCOCCOCCOCCOCCOCCC(=O)O)C(*)=O ; *CCC(=O)N[C@@H](C(*)=O)C(C)OC(=O)CCOCCOCCOCCOCCOCCOCCOCCOCCOCCC(=O)O]

For example, in the compounds of the present application, L or Lx comprises L³, and L³ may be a polypeptide residue.

For example, L³ comprises at least 1 amino acid residue.

For example, L³ comprises a residue of a hydrophobic amino acid that may be selected from the group consisting of phenylalanine (F), isoleucine (I), leucine (L), tryptophan (W), valine (V), methionine (M), tyrosine (Y), alanine (A), threonine (T), and histidine (H).

For example, L³ comprises a residue of ahydrophilic amino acid that may be selected from the group consisting of serine (S), glutamine (Q), arginine (R), lysine (K), asparagine (N), glutamic acid (E), proline (P), citrulline (C) and aspartic acid (D).

For example, L³ comprises glycine (G).

For example, L³ does not comprise a residue of a hydrophilic amino acid.

For example, L³ comprises a residue of an amino acid that may be selected from the group consisting of glycine (G), valine (V), alanine (A) and phenylalanine (F).

For example, L³ may be selected from the group consisting of: glycine-glycine-phenylalanine-glycine (GGFG), glycine-phenylalanine-glycine-glycine (GFGG), glycine-glycine-alanine-glycine (GGAG), glycine-alanine-glycine-glycine (GAGG), alanine-alanine-alanine-glycine (AAAG), glycine-alanine-alanine-alanine (GAAA), glycine-glycine-glycine-glycine (GGGG), glycine-glycine-alanine (GGA), alanine-glycine-glycine (AGG), glycine-alanine-glycine (GAG), glycine-phenylalanine-glycine (GFG), valine-alanine-glycine (VAG), glycine-alanine-valine (GAV), alanine-alanine-glycine (AAG), glycine-alanine-alanine (GAA), alanine-alanine-alanine (AAA), valine-alanine (VA), alanine-valine (AV), alanine-alanine (AA), glycine-alanine (GA), and alanine-glycine (AG).

For example, L³ may be selected from the group consisting of: glycine-phenylalanine-glycine-glycine (GFGG) and alanine-alanine (AA).

For example, L³ comprises residues of at least 1 hydrophilic amino acid.

For example, L³ comprises a residue of an amino acid that may be selected from the group consisting of: glycine (G), valine (V), alanine (A), citrulline (C), lysine (K), glutamic acid (E) and aspartic acid (D).

For example, wherein L³ may be selected from the group consisting of: glutamic acid-alanine-glycine-glycine (EAGG), glycine-glycine-alanine-glutamic acid (GGAE), glycine-glutamic acid-alanine-glycine (GEAG), glycine-alanine-glutamic acid-glycine (GAEG), glycine-aspartic acid-alanine-glycine (GDAG), glycine-alanine-aspartic acid-glycine (GDAG), glycine-aspartic acid-glycine-glycine (GDGG), glycine-glycine-aspartic acid-glycine (GGDG), glycine-glutamic acid-glycine-glycine (GEGG), glycine-glycine-glutamic acid-glycine (GGEG), glutamic acid-glycine-glycine (EGG), glycine-glycine-glutamic acid (GGE), glutamic acid-alanine-glycine (EAG), glycine-alanine-glutamic acid (GAE), aspartic acid-alanine-glycine (DAG), glycine-alanine-aspartic acid (GAD), aspartic acid-glycine-glycine (DGG), glycine-glycine-aspartic acid (GGD), valine-lysine-glycine (VKG), glycine-lysine-valine (GKV), glycine-aspartic acid-glycine (GDG), glycine-aspartic acid-alanine (GDA), alanine-aspartic acid-glycine (ADG), glycine-glutamic acid-glycine (GEG), valine-citrulline-glycine (VCG), glycine-citrulline-valine (GCV), glycine-glutamic acid-alanine (GEA), alanine-glutamic acid-glycine (AEG), glutamic acid-alanine (EA), alanine-glutamic acid (AE), glutamic acid-glycine (EG), glycine-glutamic acid (GE), valine-citrulline (VC), citrulline-valine (CV), aspartic acid-alanine (DA), alanine-aspartic acid (AD), aspartic acid-glycine (DG), glycine-aspartic acid (GD), lysine-valine (KV) and valine-lysine (VK).

For example, L³ may be selected from the group consisting of: glycine-glutamic acid-glycine (GEG), glycine-alanine-glutamic acid (GAE), alanine-glutamic acid (AE), glutamic acid-glycine (EG), and glycine-glutamic acid (GE).

For example, L³ does not comprise a residue of a hydrophobic amino acid.

For example, L³ comprises a residue of an amino acid that may be selected from the group consisting of: glycine (G), glutamic acid (E), and aspartic acid (D).

For example, L³ may be selected from the group consisting of: glycine-glutamic acid (GE), glutamic acid-glycine (EG), glycine-glycine (GG), aspartic acid-glycine (DG), glycine-aspartic acid (GD), glycine-aspartic acid-glycine (GDG), aspartic acid-glycine-glycine (DGG), glycine-glycine-aspartic acid (GGD), glycine-glutamic acid-glycine (GEG), glycine-glycine-aspartic acid-glycine (GGDG), and glycine-aspartic acid-glycine-glycine (GDGG).

For example, L³ may be selected from the group consisting of: glutamic acid-glycine (EG), glycine-glutamic acid (GE), glutamic acid-alanine (EA), alanine-glutamic acid (AE), valine-citrulline (VC), citrulline-valine (CV), valine-alanine (VA), alanine-valine (AV), alanine-alanine (AA), glutamic acid-alanine-glycine (EAG), glycine-alanine-glutamic acid (GAE), glutamic acid-glycine-glycine (EGG), glycine-glycine-glutamic acid (GGE), glycine-glutamic acid-glycine (GEG), alanine-alanine-glycine (AAG), glycine-alanine-alanine (GAA), alanine-alanine-alanine (AAA), valine-alanine-glycine (VAG), glycine-alanine-valine (GAV), valine-citrulline-glycine (VCG), glycine-citrulline-valine (GCV), valine-lysine-glycine (VKG), glycine-lysine-valine (GKV), glycine-glycine-phenylalanine-glycine (GGFG), glycine-phenylalanine-glycine-glycine (GFGG), glycine-glycine-glycine-glycine (GGGG), glycine-glutamic acid-glycine-glycine (GEGG), glycine-glycine-glutamic acid-glycine (GGEG), glycine-alanine-glutamic acid-glycine (GAEG) and glycine-glutamic acid-alanine-glycine (GEAG).

For example, L³ may be selected from the group consisting of: alanine-alanine (AA), glycine-alanine-glutamic acid (GAE), glycine-glutamic acid-glycine (GEG), alanine-glutamic acid (AE), glutamic acid-glycine (EG), and glycine-glutamic acid (GE).

For example, L may comprise -L³-L²-L¹- or -L³-L¹-.
preferably, L is selected from

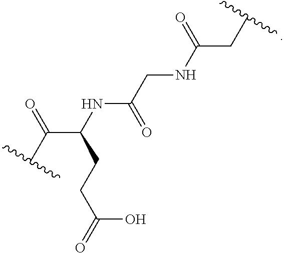 [SMILES: *CC(=O)NCC(=O)N[C@@H](CCC(=O)O)C(*)=O]

For example, Lx may comprise -L³-L²-L^{1x} or -L³-L^{1X};
preferably, Lx is selected from

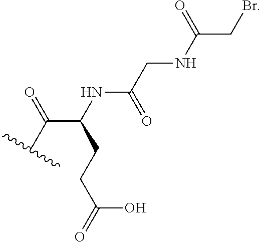 [SMILES: *C(=O)[C@H](CCC(=O)O)NC(=O)CNC(=O)CBr]

For example, in the compounds of the present application, Ab comprises an antibody or an antigen-binding fragment thereof:

For example, in the compounds of the present application, the antibody may be selected from the group consisting of: a murine antibody, a chimeric antibody, a humanized antibody and a fully humanized antibody.

For example, in the compounds of the present application, the antigen-binding fragment may be selected from the group consisting of: a Fab, a Fab′, an Fv fragment, a F(ab′)₂, a F(ab)₂, a scFv, a di-scFv, a VHH and a dAb.

For example, in the compounds of the present application, N^{a-1} may be a number from about 0 to about 20.

For example, in the compounds of the present application, Ab targets a target that may be selected from the group consisting of:
AXL, BAFFR, BCMA, BCR-list components, BDCA2, BDCA4, BTLA, BTNL2 BTNL3, BTNL8, BTNL9, C10orf54, CCR1, CCR3, CCR4, CCR5, CCR6, CCR7, CCR9, CCR10, CD11c, CD137, CD138, CD14, CD163, CD168, CD177, CD19, CD20, CD209, CD209L, CD22, CD226, CD248, CD25, CD27, CD274, CD276, CD28, CD30, CD300A, CD33, CD37, CD38, CD4, CD40, CD44, CD45, CD46, CD47, CD48, CD5, CD52, CD55, CD56, CD59, CD62E, CD68, CD69, CD70, CD74, CD79a, CD79b, CD8, CD80, CD86, CD90.2, CD96, CLEC12A, CLEC12B, CLEC7A, CLEC9A, CR1, CR3, CRTAM, CSF1R, CTLA4, CXCR1/2, CXCR4, CXCR5, DDR1, DDR2, DEC-205, DLL4, DR6, FAP, FCamR, FCMR, FcR's, Fire, GITR, HHLA2, HLA class II, HVEM, ICOSLG, IFNAR, IFNLRT, IL10R1, IL10R2, IL12R, IL13RA1, IL13RA2, IL15R, IL17RA, IL17RB, IL17RC, IL17RE, IL20R1, IL20R2, IL21R, IL22R1, IL22RA, IL23R, IL27R, IL29R, IL2Rg, IL31R, IL36R, IL3RA, IL4R, IL6R, IL5R, IL7R, IL9R, integrins, LAG3, LIFR, MAG/Siglec-4 (sialic acid-binding immunoglobulin-like lectin-4), MMR, MSR1, NCR3LG1, NKG2D, NKp30, NKp46, OX40 (CD134), PDCD1, PROKR1, PVR, PVRIG, PVRL2, PVRL3, RELT, SIGIRR, Siglec-1 (sialic acid-binding immunoglobulin-like lectin-1), Siglec-10, Siglec-5, Siglec-6, Siglec-7, Siglec-8, Siglec-9, SIRPA, SLAMF7, TACI, TCR-list components/assoc, PTCRA, TCRb, CD3z, CD3, TEK, TGFBR1, TGFBR2, TGFBR3, TIGIT, TLR2, TLR4, TNFα, TROY, TSLPR, TYRO, VLDLR, VSIG4, IL2R-y and VTCN1.

For example, Ab may be selected from the group consisting of: an anti-TNFα antibody or an antigen-binding fragment thereof, an anti-CD40 antibody or an antigen-binding fragment thereof, an anti-BDCA2 antibody or an antigen-binding fragment thereof, and an anti-IFNAR1 antibody or an antigen-binding fragment thereof:

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-A), formula (I-a2-A), formula (I-a3-A) or formula (I-a4-A):

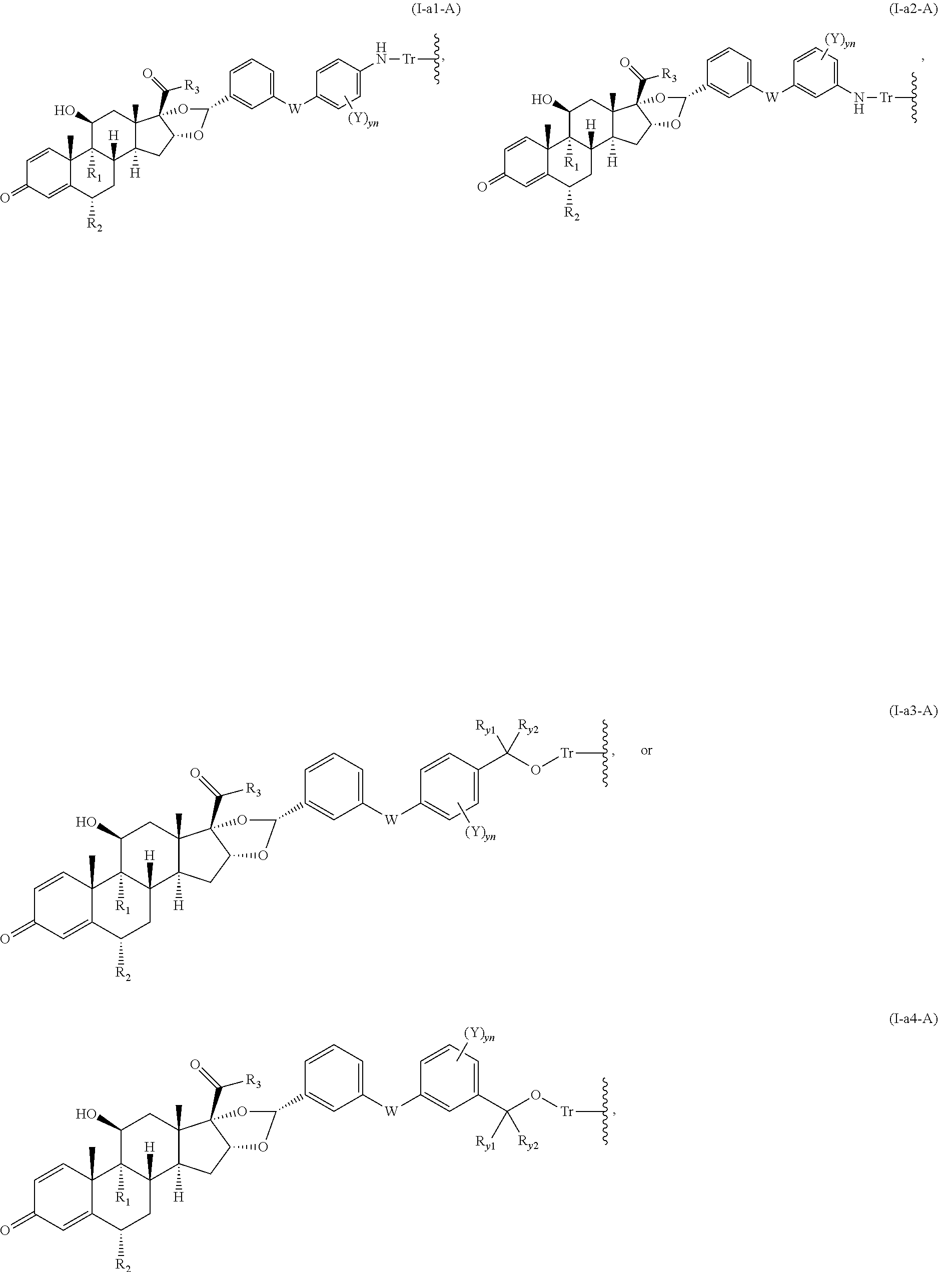

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen, halogen, and optionally substituted methyl, R₂ may be selected from the group consisting of hydrogen, halogen, and optionally substituted methyl, R₃ may be selected from the group consisting of optionally substituted —CH₂Cl, optionally substituted —CH₂SH, optionally substituted —CH₂OH, optionally substituted

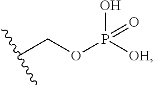 [SMILES: *COP(=O)(O)O]

optionally substituted

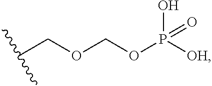 [SMILES: *COCOP(=O)(O)O]

optionally substituted

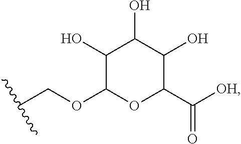 [SMILES: *COC1OC(C(=O)O)C(O)C(O)C1O]

optionally substituted

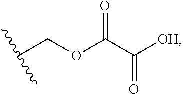 [SMILES: *COC(=O)C(=O)O]

optionally substituted

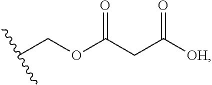 [SMILES: *COC(=O)CC(=O)O]

optionally substituted

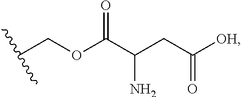 [SMILES: *COC(=O)C(N)CC(=O)O]

optionally substituted

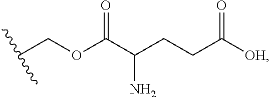 [SMILES: *COC(=O)C(N)CCC(=O)O]

optionally substituted

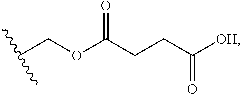 [SMILES: *COC(=O)CCC(=O)O]

optionally substituted

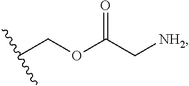 [SMILES: *COC(=O)CN]

optionally substituted

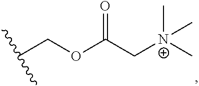 [SMILES: *COC(=O)C[N+](C)(C)C]

optionally substituted

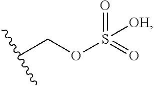 [SMILES: *COS(=O)(=O)O]

optionally substituted

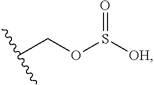 [SMILES: *COS(=O)O]

optionally substituted

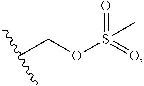 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Tr may be absent or may be selected from the group consisting of:

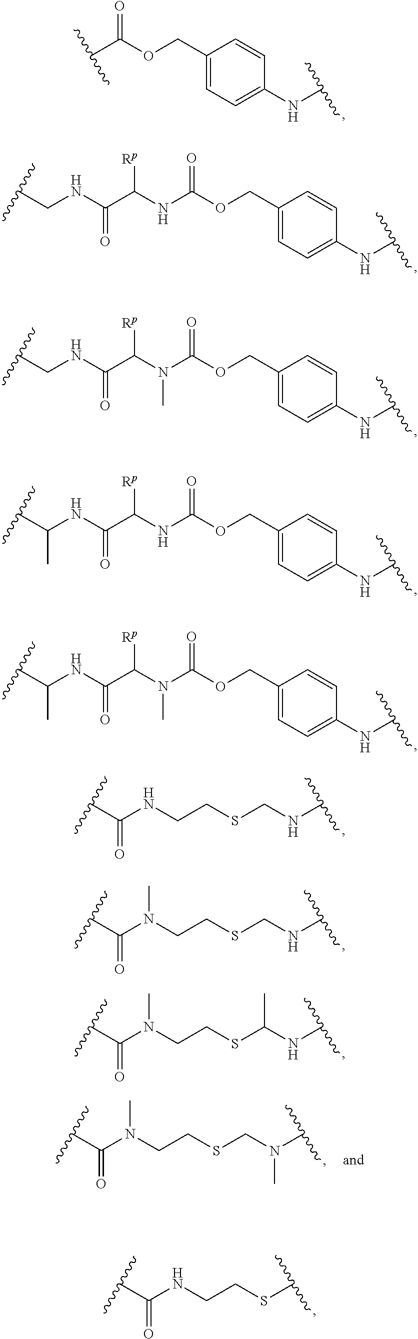

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-A), formula (I-a2-A), formula (I-a3-A) or formula (I-a4-A):

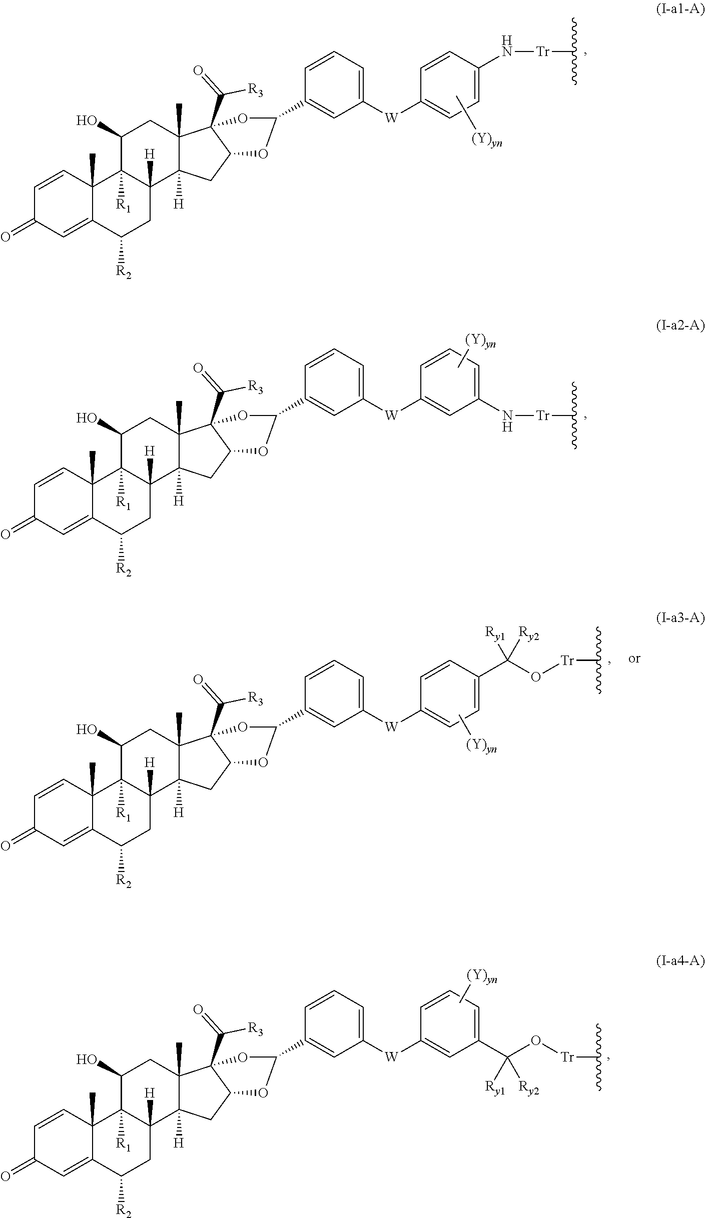

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂F

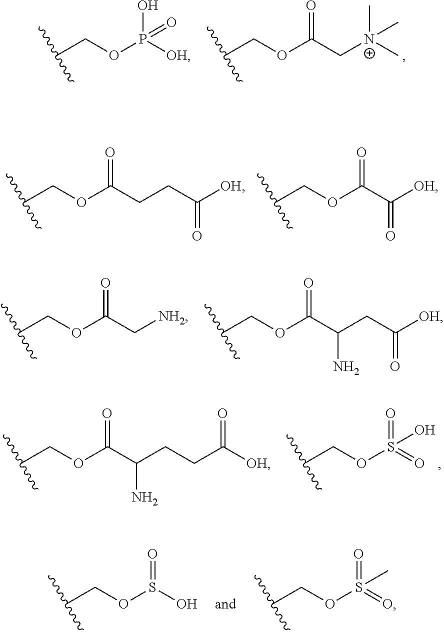

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-A), formula (II-a2-A), formula (II-a3-A) or formula (II-a4-A):

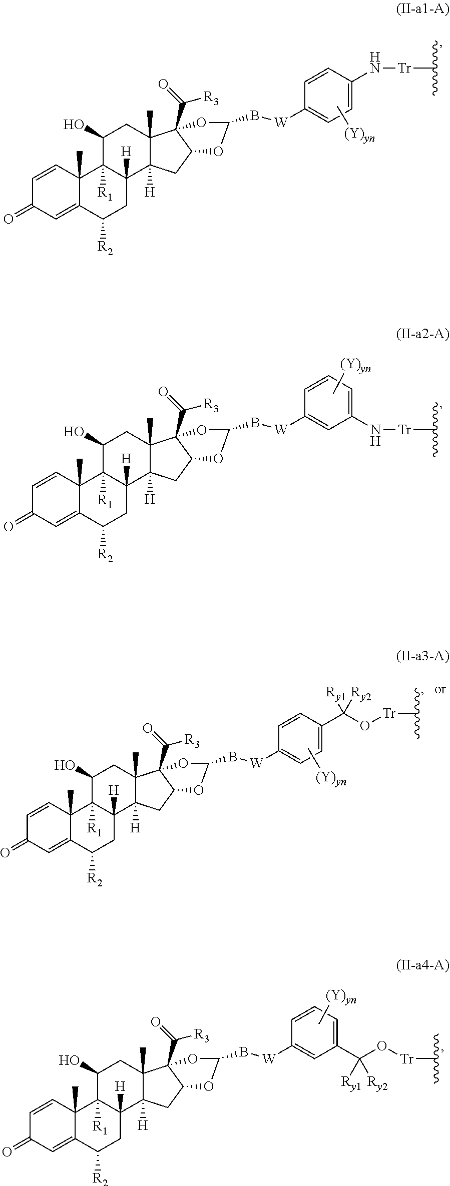

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

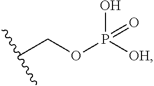 [SMILES: *COP(=O)(O)O]

optionally substituted

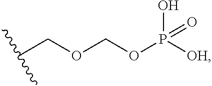 [SMILES: *COCOP(=O)(O)O]

optionally substituted

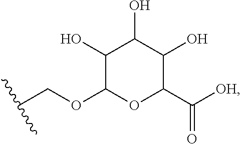 [SMILES: *COC1OC(C(=O)O)C(O)C(O)C1O]

optionally substituted

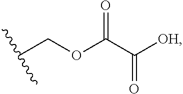 [SMILES: *COC(=O)C(=O)O]

optionally substituted

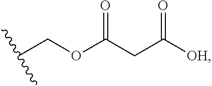 [SMILES: *COC(=O)CC(=O)O]

optionally substituted

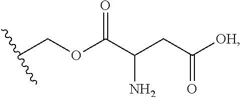 [SMILES: *COC(=O)C(N)CC(=O)O]

optionally substituted

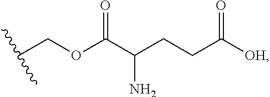 [SMILES: *COC(=O)C(N)CCC(=O)O]

optionally substituted

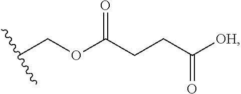 [SMILES: *COC(=O)CCC(=O)O]

optionally substituted

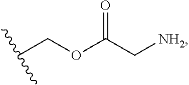 [SMILES: *COC(=O)CN]

optionally substituted

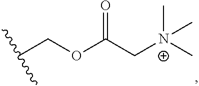 [SMILES: *COC(=O)C[N+](C)(C)C]

optionally substituted

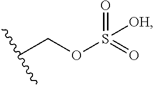 [SMILES: *COS(=O)(=O)O]

optionally substituted

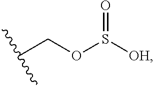 [SMILES: *COS(=O)O]

optionally substituted

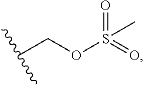 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

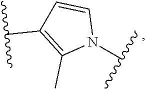 [SMILES: *c1ccn(*)c1C]

optionally substituted

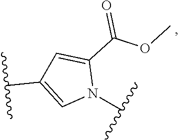 [SMILES: *c1cc(C(=O)OC)n(*)c1]

optionally substituted

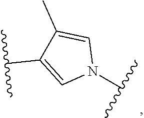 [SMILES: *c1cn(*)cc1C]

optionally substituted

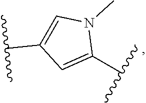 [SMILES: *c1cc(*)n(C)c1]

optionally substituted

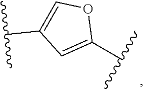 [SMILES: *c1coc(*)c1]

optionally substituted

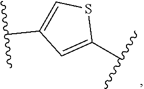 [SMILES: *c1csc(*)c1]

optionally substituted

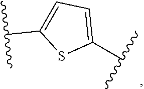 [SMILES: *c1ccc(*)s1]

optionally substituted

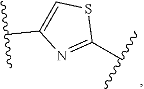 [SMILES: *c1csc(*)n1]

and optionally substituted

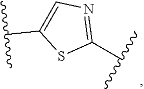 [SMILES: *c1cnc(*)s1]

W may be absent or W may be selected from the group consisting of the following structures that are optionally substituted:

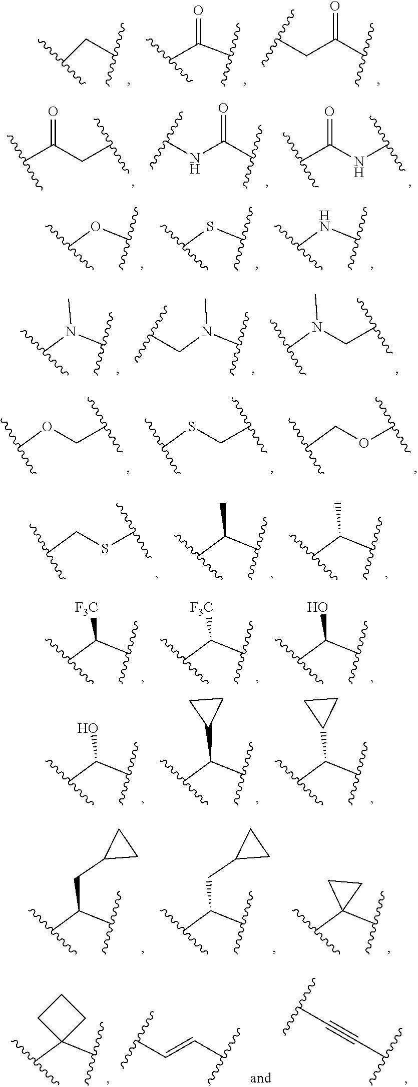

Y may be H, and yn may be a number greater than 0;
Tr may be absent or may be selected from the group consisting of:

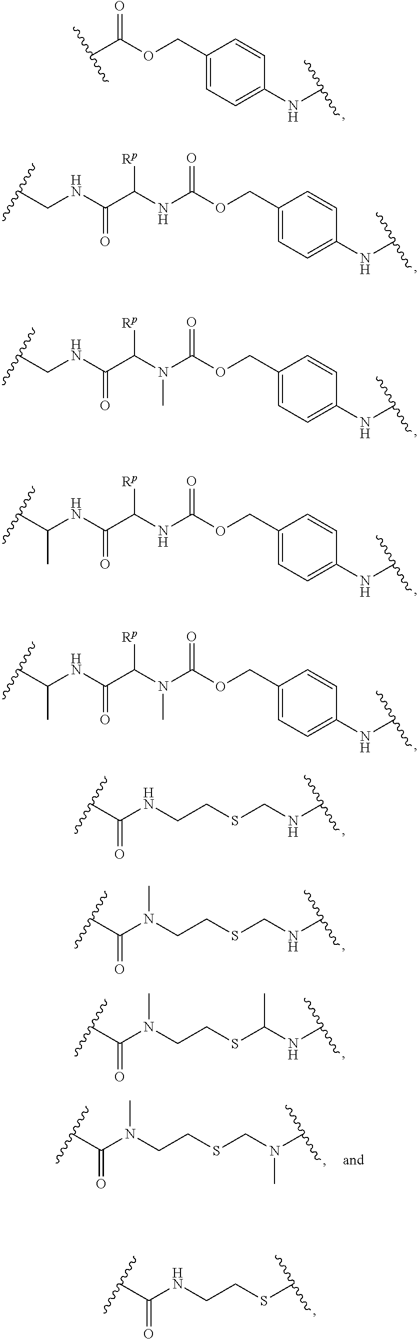

wherein R^{P} may be selected from the group consisting of: H, —CH₃, —CH—(CH₃)₂, —CH₂—CH(CH₃)₂, —CH(CH₃)—CH₂—CH₃, —CH₂—C₆H₅, —CsNH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-A), formula (II-a2-A), formula (II-a3-A) or formula (II-a4-A):

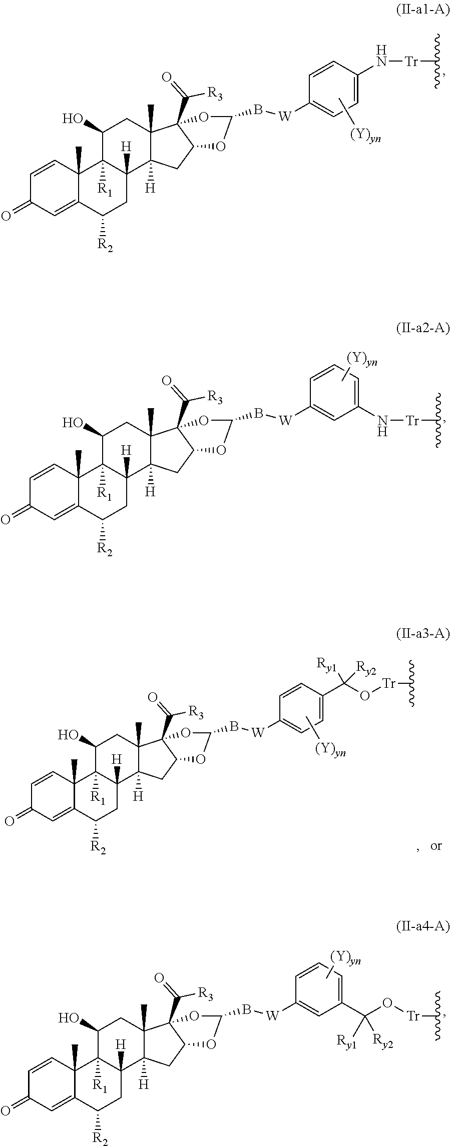

wherein

Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂-halogen,

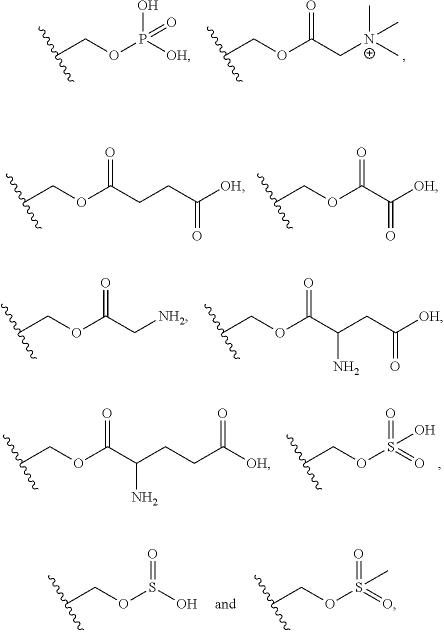

B may be selected from the group consisting of: optionally substituted

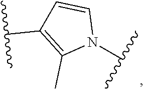 [SMILES: *c1ccn(*)c1C]

optionally substituted

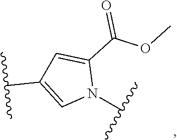 [SMILES: *c1cc(C(=O)OC)n(*)c1]

optionally substituted

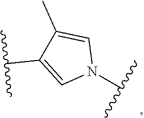 [SMILES: *c1cn(*)cc1C]

optionally substituted

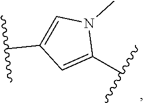 [SMILES: *c1cc(*)n(C)c1]

optionally substituted

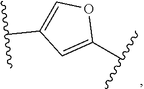 [SMILES: *c1coc(*)c1]

optionally substituted

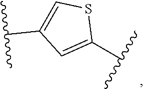 [SMILES: *c1csc(*)c1]

optionally substituted

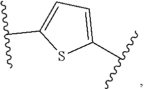 [SMILES: *c1ccc(*)s1]

optionally substituted

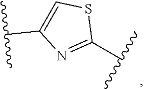 [SMILES: *c1csc(*)n1]

and optionally substituted

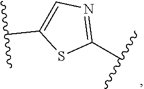 [SMILES: *c1cnc(*)s1]

W may be absent or W may be selected from the group consisting of the following structures that are optionally substituted: optionally substituted —CH₂—, optionally substituted —CH(CH₃)—, and optionally substituted

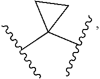 [SMILES: *C1(*)CC1]

Y may be H, and yn may be a number greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-A), formula (II-b2-A), formula (II-b3-A) or formula (II-b4-A):

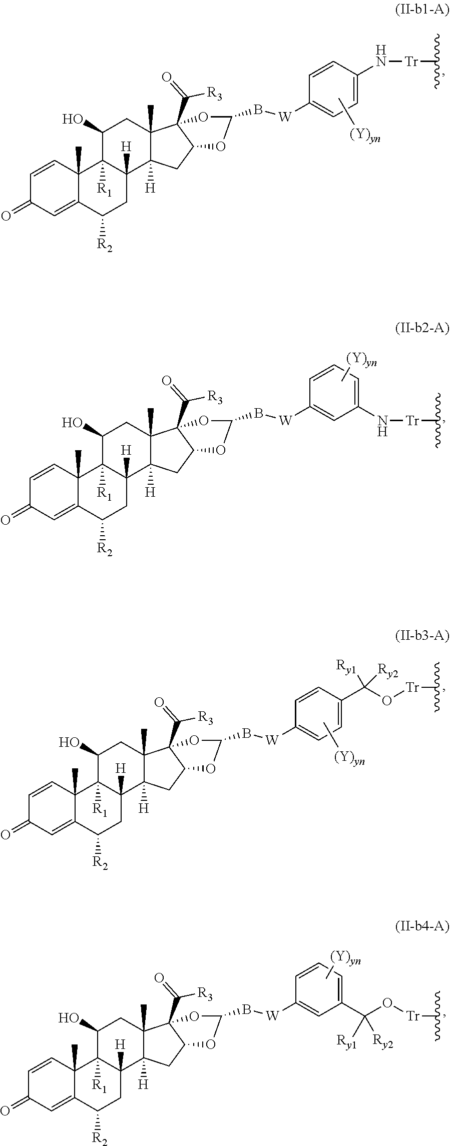

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

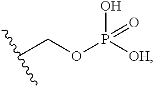 [SMILES: *COP(=O)(O)O]

optionally substituted

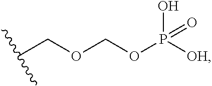 [SMILES: *COCOP(=O)(O)O]

optionally substituted

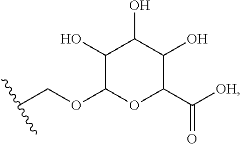 [SMILES: *COC1OC(C(=O)O)C(O)C(O)C1O]

optionally substituted

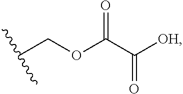 [SMILES: *COC(=O)C(=O)O]

optionally substituted

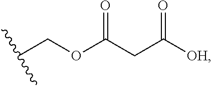 [SMILES: *COC(=O)CC(=O)O]

optionally substituted

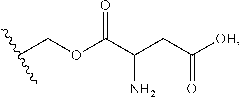 [SMILES: *COC(=O)C(N)CC(=O)O]

optionally substituted

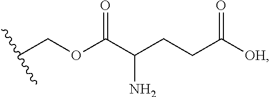 [SMILES: *COC(=O)C(N)CCC(=O)O]

optionally substituted

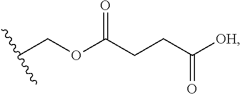 [SMILES: *COC(=O)CCC(=O)O]

optionally substituted

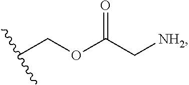 [SMILES: *COC(=O)CN]

optionally substituted

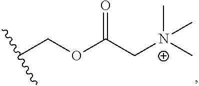 [SMILES: *COC(=O)C[N+](C)(C)C]

optionally substituted

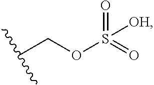 [SMILES: *COS(=O)(=O)O]

optionally substituted

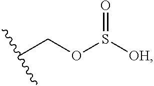 [SMILES: *COS(=O)O]

optionally substituted

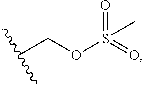 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

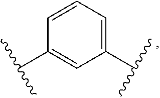 [SMILES: *c1cccc(*)c1]

optionally substituted

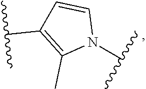 [SMILES: *c1ccn(*)c1C]

optionally substituted

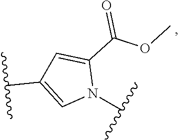 [SMILES: *c1cc(C(=O)OC)n(*)c1]

optionally substituted

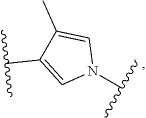 [SMILES: *c1cn(*)cc1C]

optionally substituted

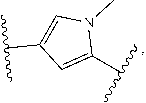 [SMILES: *c1cc(*)n(C)c1]

optionally substituted

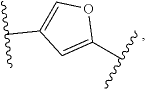 [SMILES: *c1coc(*)c1]

optionally substituted

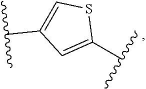 [SMILES: *c1csc(*)c1]

optionally substituted

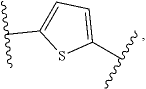 [SMILES: *c1ccc(*)s1]

optionally substituted

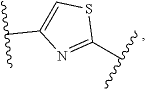 [SMILES: *c1csc(*)n1]

and optionally substituted

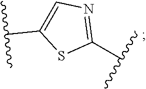 [SMILES: *c1cnc(*)s1]

W may be absent or W may be selected from the group consisting of the following structures that are optionally substituted:

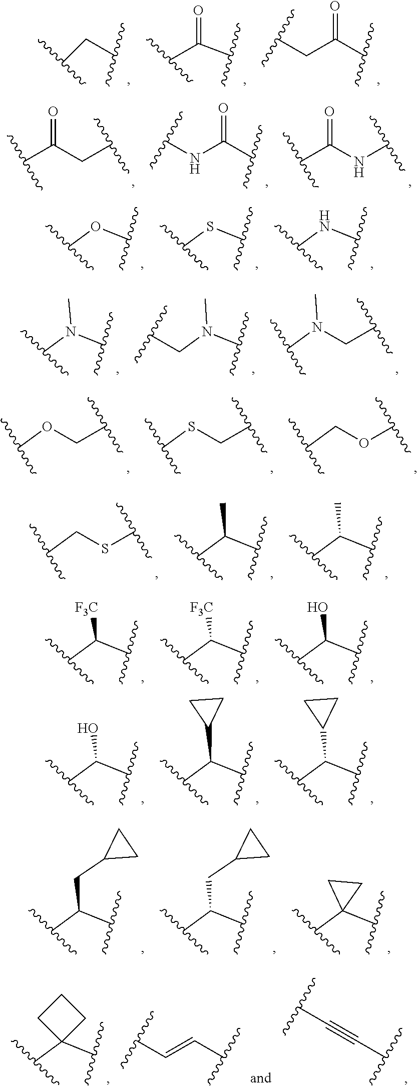

wherein when B may be optionally substituted

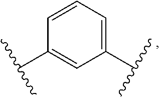 [SMILES: *c1cccc(*)c1]

### W may not be —S—;

Y may be H, and yn may be a number greater than 0;
Tr may be absent or may be selected from the group consisting of:

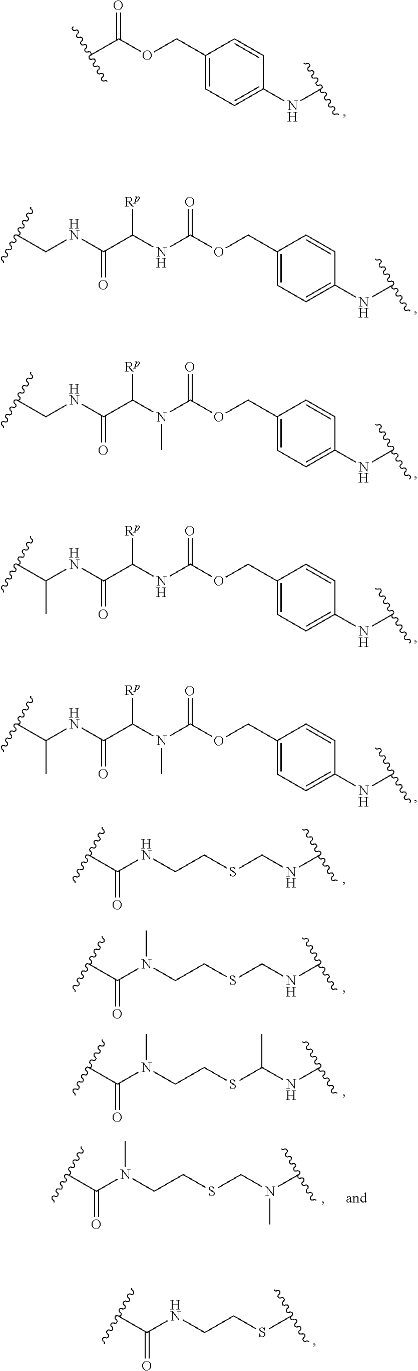

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH2, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-A), formula (II-b2-A), formula (II-b3-A) or formula (II-b4-A):

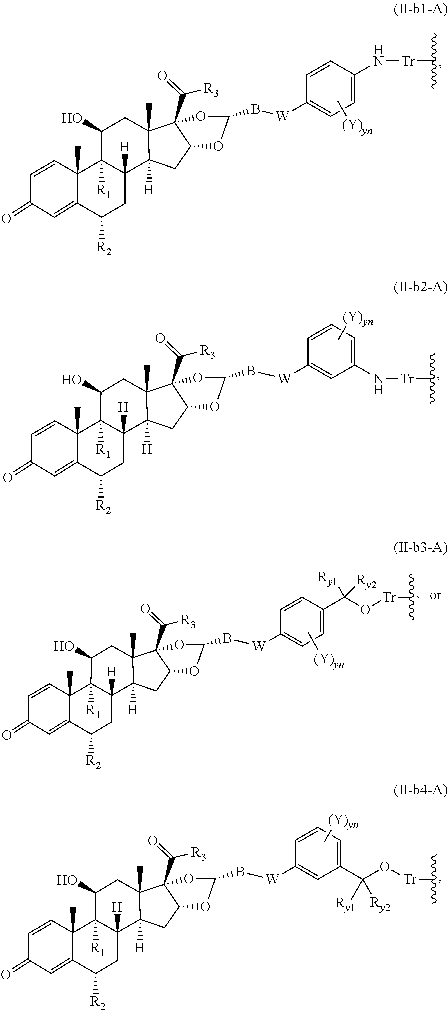

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen and halogen, R₂ may be selected from the group consisting of: hydrogen and halogen, R₃ may be selected from the group consisting of —CH₂OH, —SCH₂-halogen,

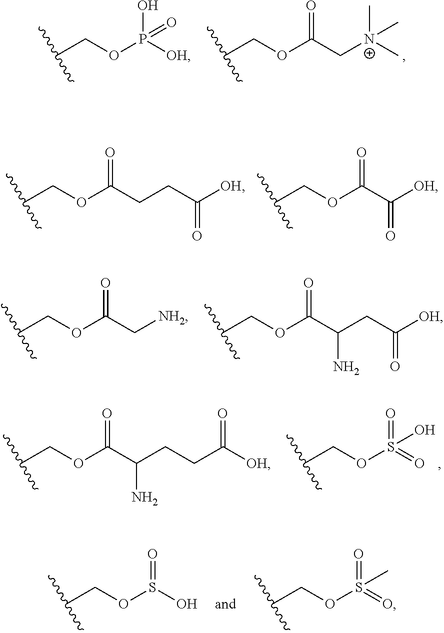

B may be selected from the group consisting of optionally substituted

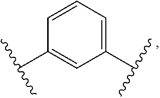 [SMILES: *c1cccc(*)c1]

optionally substituted

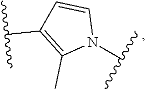 [SMILES: *c1ccn(*)c1C]

optionally substituted

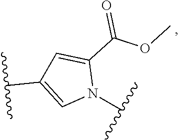 [SMILES: *c1cc(C(=O)OC)n(*)c1]

optionally substituted

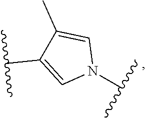 [SMILES: *c1cn(*)cc1C]

optionally substituted

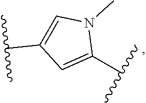 [SMILES: *c1cc(*)n(C)c1]

optionally substituted

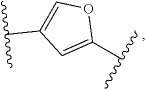 [SMILES: *c1coc(*)c1]

optionally substituted

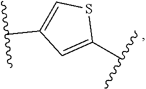 [SMILES: *c1csc(*)c1]

optionally substituted

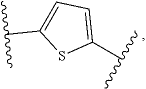 [SMILES: *c1ccc(*)s1]

optionally substituted

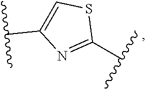 [SMILES: *c1csc(*)n1]

and optionally substituted;

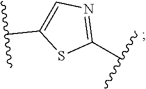 [SMILES: *c1cnc(*)s1]

W may be absent or W may be selected from the group consisting of the following structures that are optionally substituted: optionally substituted —CH₂—, optionally substituted —CH(CH₃)—, and optionally substituted

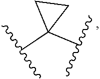 [SMILES: *C1(*)CC1]

Y may be H, and yn may be a number greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-B), formula (I-a2-B), formula (I-a3-B) or formula (I-a4-B):

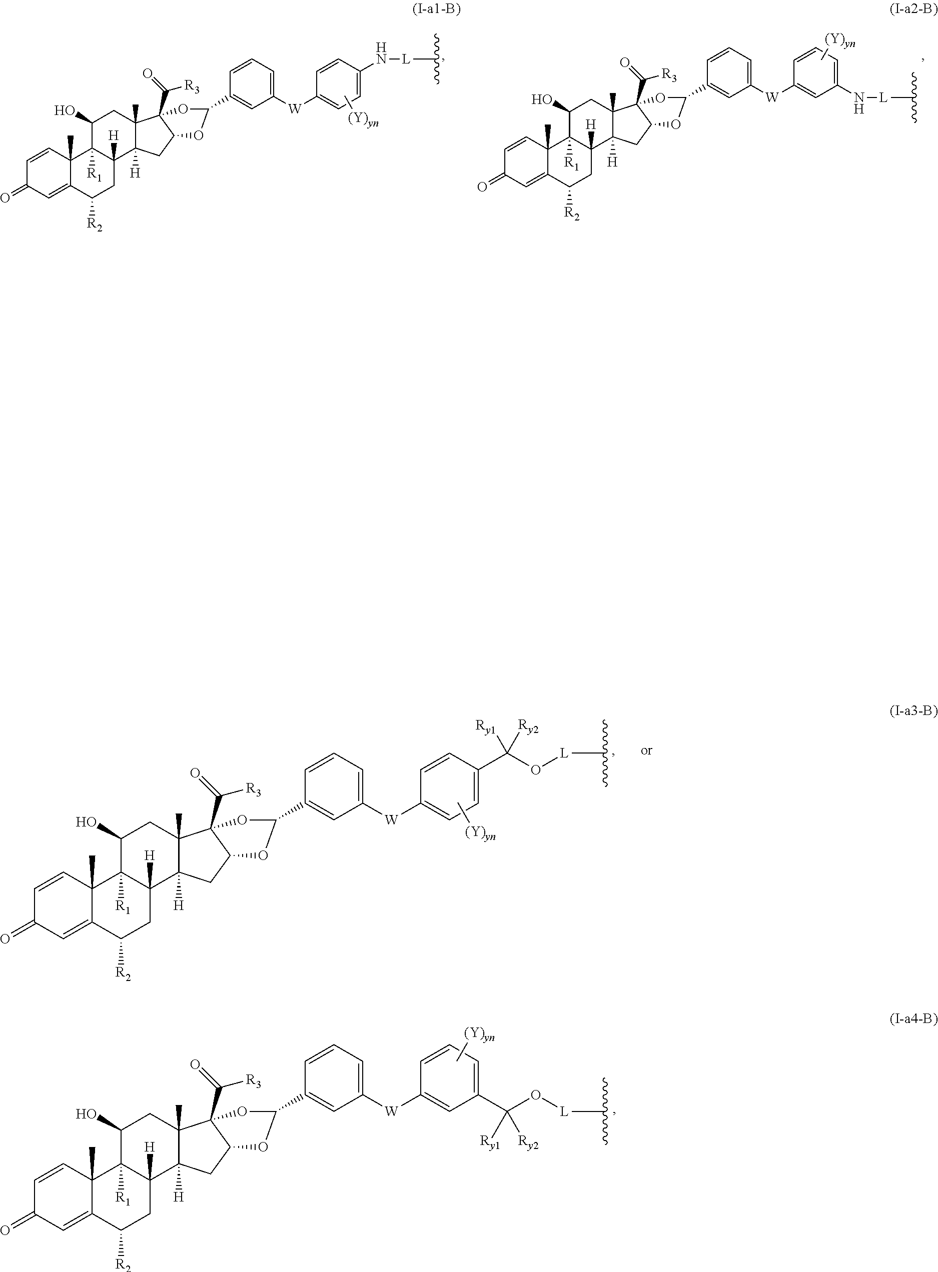

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

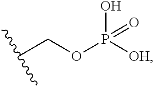 [SMILES: *COP(=O)(O)O]

optionally substituted

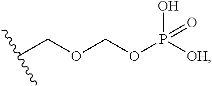 [SMILES: *COCOP(=O)(O)O]

optionally substituted

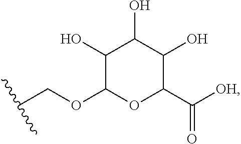 [SMILES: *COC1OC(C(=O)O)C(O)C(O)C1O]

optionally substituted

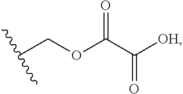 [SMILES: *COC(=O)C(=O)O]

optionally substituted

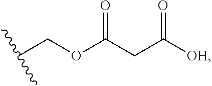 [SMILES: *COC(=O)CC(=O)O]

optionally substituted

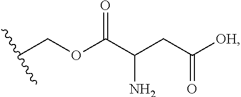 [SMILES: *COC(=O)C(N)CC(=O)O]

optionally substituted

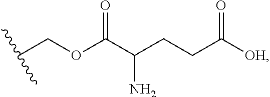 [SMILES: *COC(=O)C(N)CCC(=O)O]

optionally substituted

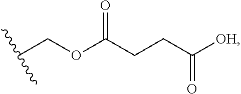 [SMILES: *COC(=O)CCC(=O)O]

optionally substituted

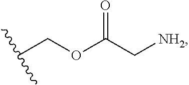 [SMILES: *COC(=O)CN]

optionally substituted

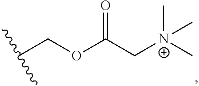 [SMILES: *COC(=O)C[N+](C)(C)C]

optionally substituted

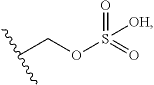 [SMILES: *COS(=O)(=O)O]

optionally substituted

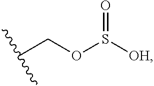 [SMILES: *COS(=O)O]

optionally substituted

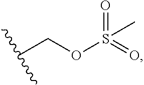 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of

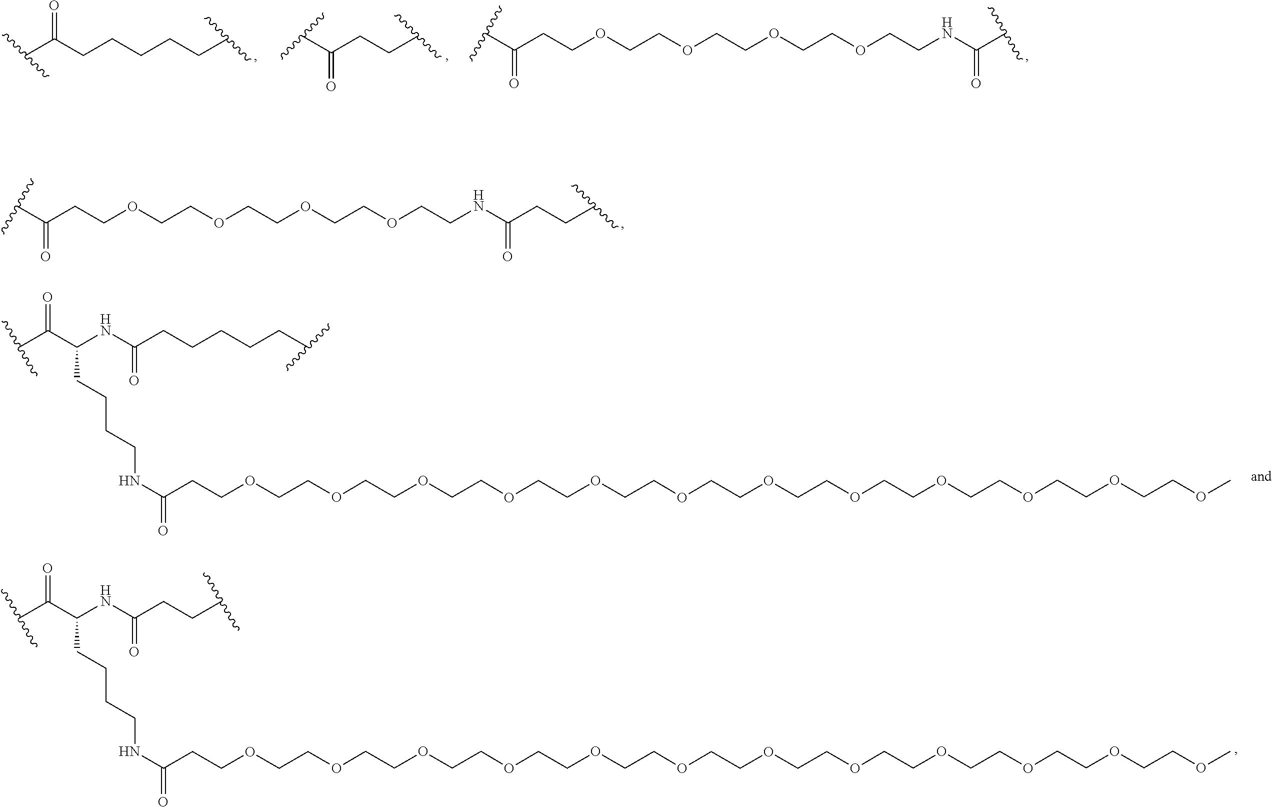

L may be selected from the group consisting of:

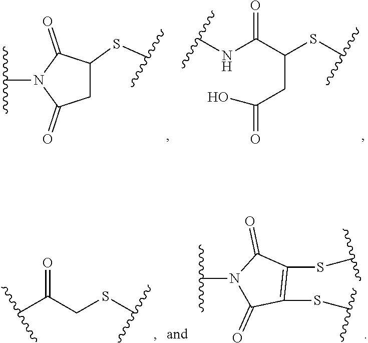 [SMILES: *SC1CC(=O)N(*)C1=O ; *NC(=O)C(CC(=O)O)S* ; *SCC(*)=O ; *SC1=C(S*)C(=O)N(*)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-B), formula (I-a2-B), formula (I-a3-B) or formula (I-a4-B):

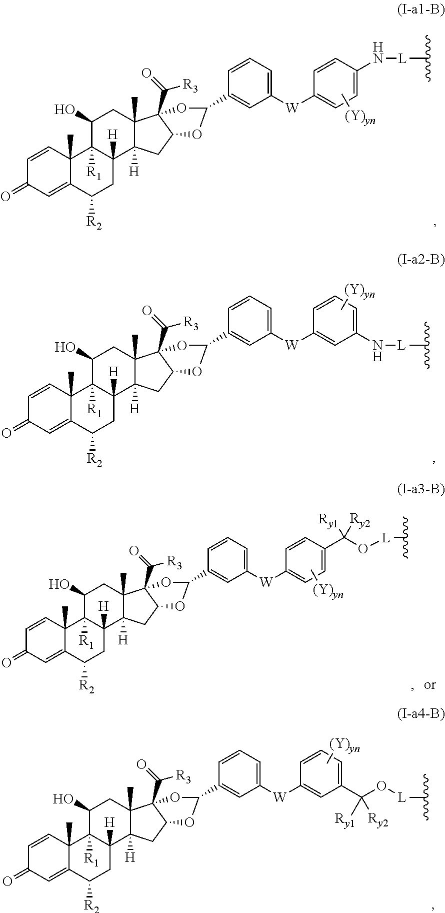

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂F,

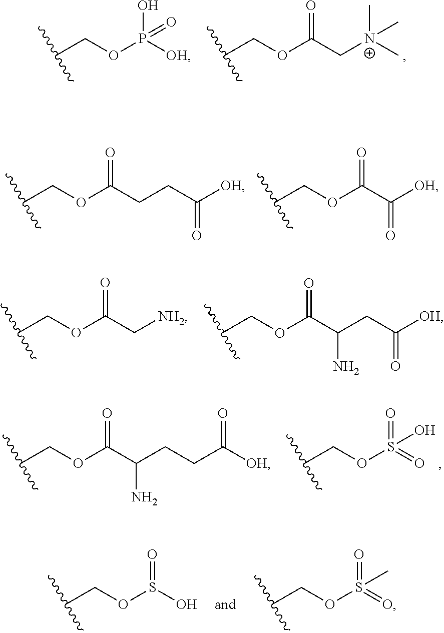

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

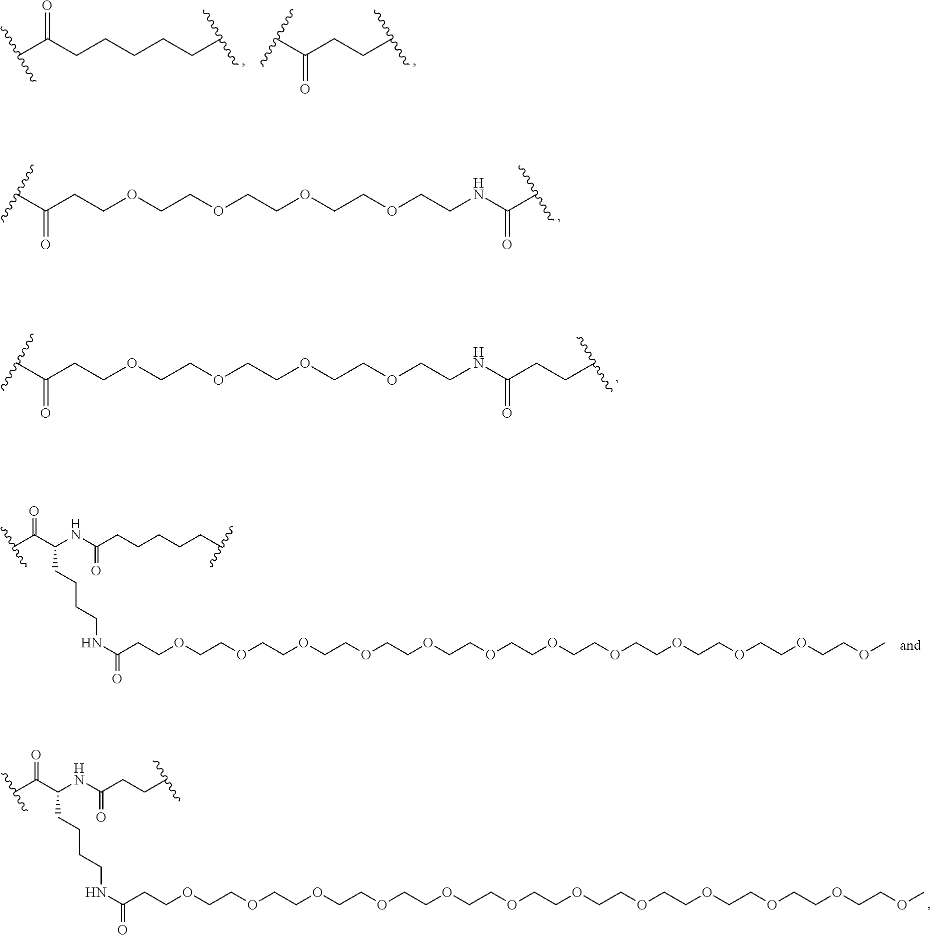 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

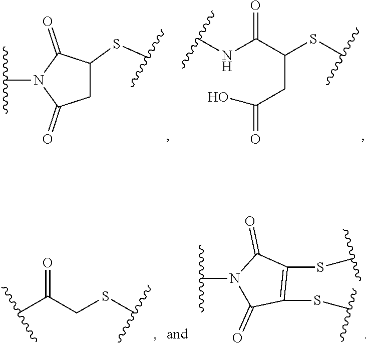 [SMILES: *SC1CC(=O)N(*)C1=O ; *NC(=O)C(CC(=O)O)S* ; *SCC(*)=O ; *SC1=C(S*)C(=O)N(*)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-B), formula (II-a2-B), formula (II-a3-B) or formula (II-a4-B):

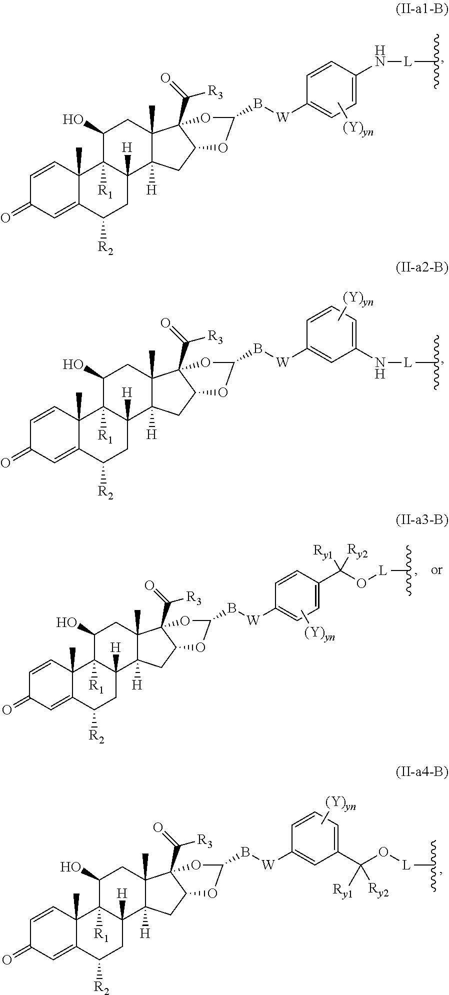

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

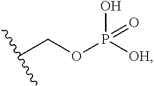 [SMILES: *COP(=O)(O)O]

optionally substituted

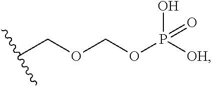 [SMILES: *COCOP(=O)(O)O]

optionally substituted

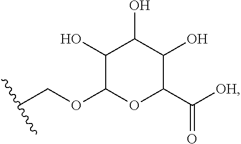 [SMILES: *COC1OC(C(=O)O)C(O)C(O)C1O]

optionally substituted

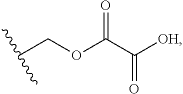 [SMILES: *COC(=O)C(=O)O]

optionally substituted

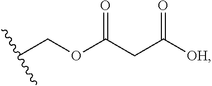 [SMILES: *COC(=O)CC(=O)O]

optionally substituted

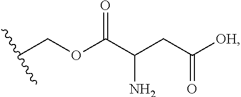 [SMILES: *COC(=O)C(N)CC(=O)O]

optionally substituted

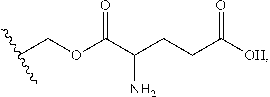 [SMILES: *COC(=O)C(N)CCC(=O)O]

optionally substituted

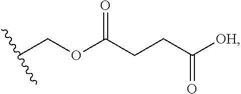 [SMILES: *COC(=O)CCC(=O)O]

optionally substituted

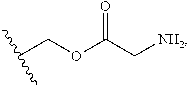 [SMILES: *COC(=O)CN]

optionally substituted

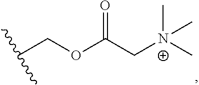 [SMILES: *COC(=O)C[N+](C)(C)C]

optionally substituted

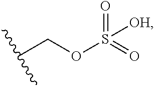 [SMILES: *COS(=O)(=O)O]

optionally substituted

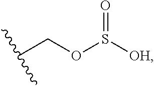 [SMILES: *COS(=O)O]

optionally substituted

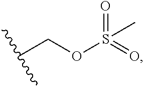 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

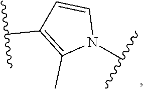 [SMILES: *c1ccn(*)c1C]

optionally substituted

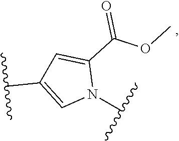 [SMILES: *c1cc(C(=O)OC)n(*)c1]

optionally substituted

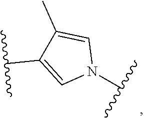 [SMILES: *c1cn(*)cc1C]

optionally substituted

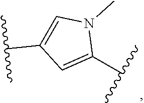 [SMILES: *c1cc(*)n(C)c1]

optionally substituted

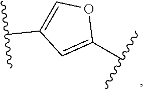 [SMILES: *c1coc(*)c1]

optionally substituted

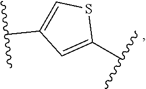 [SMILES: *c1csc(*)c1]

optionally substituted

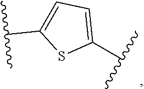 [SMILES: *c1ccc(*)s1]

optionally substituted

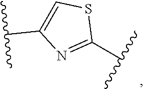 [SMILES: *c1csc(*)n1]

and optionally substituted

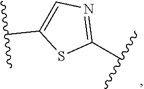 [SMILES: *c1cnc(*)s1]

W may be absent or W may be selected from the group consisting of the following structures that are optionally substituted:

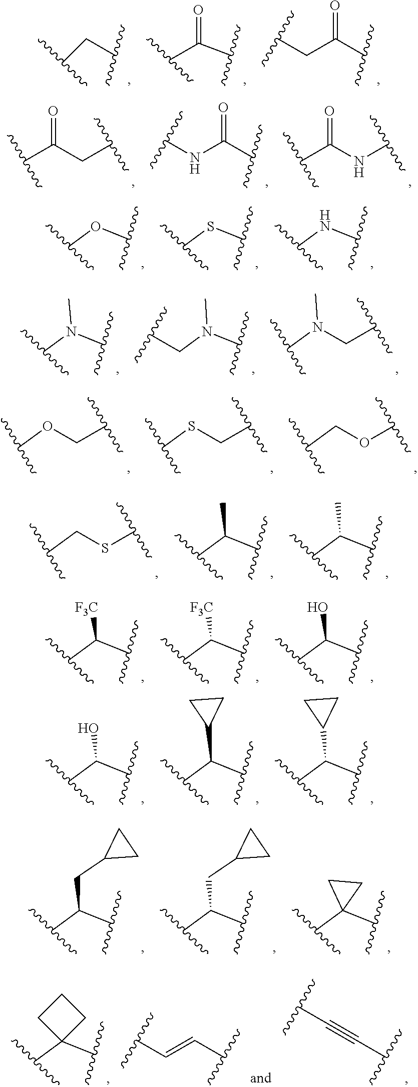

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

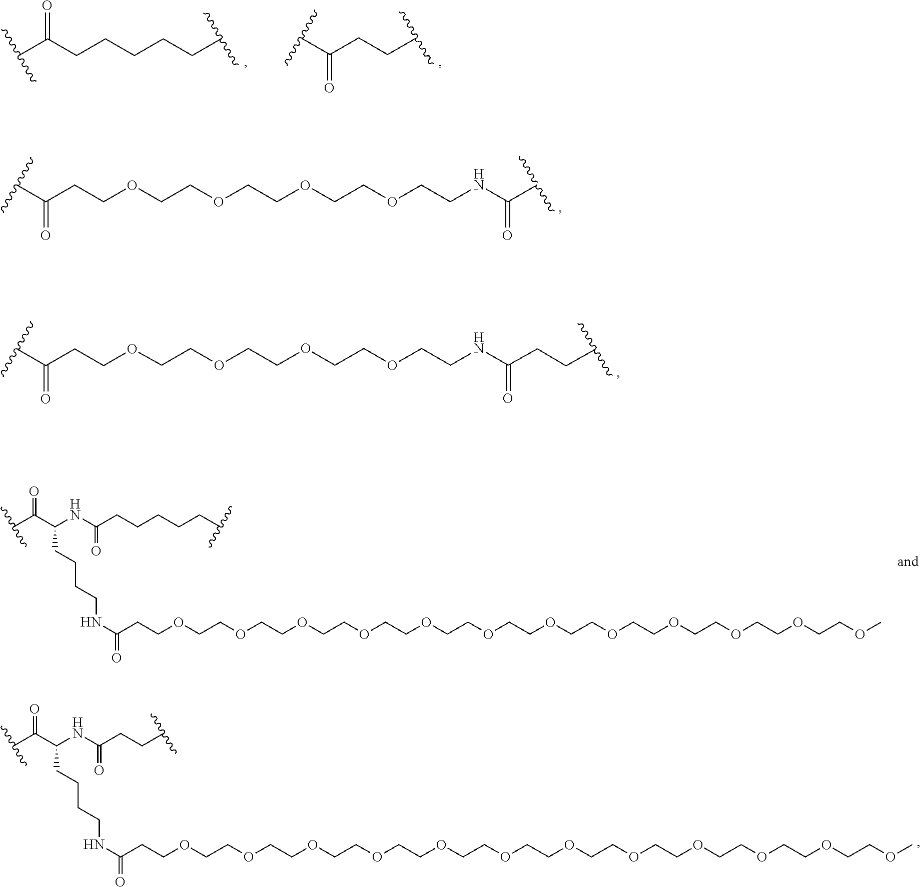 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L may be selected from the group consisting of:

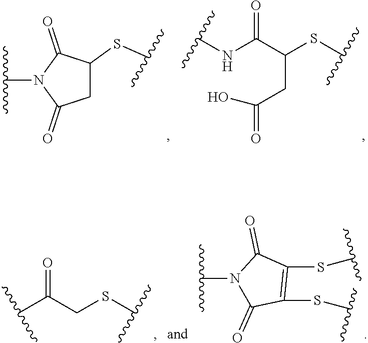 [SMILES: *SC1CC(=O)N(*)C1=O ; *NC(=O)C(CC(=O)O)S* ; *SCC(*)=O ; *SC1=C(S*)C(=O)N(*)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-B), formula (II-a2-B), formula (II-a3-B) or formula (II-a4-B):

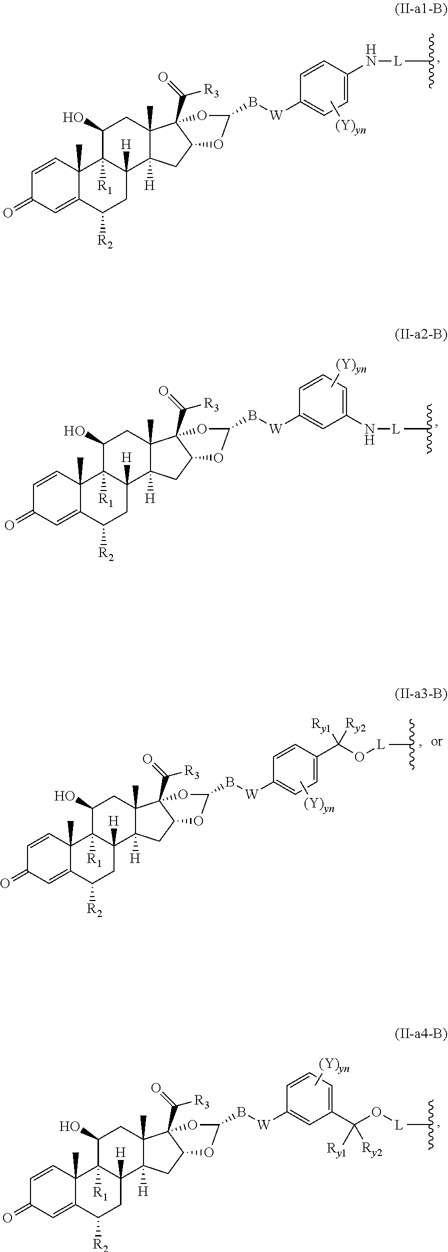

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂-halogen,

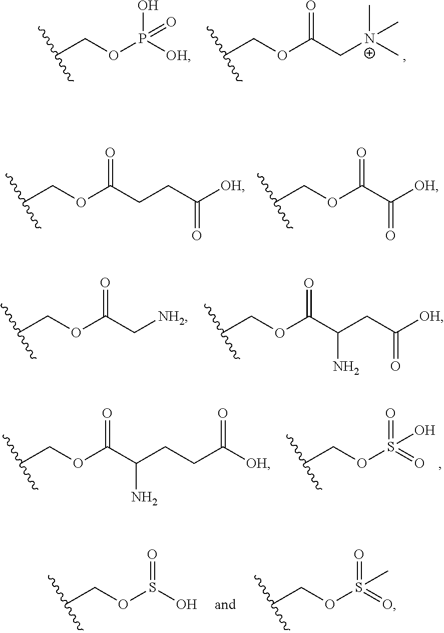

B may be selected from the group consisting of: optionally substituted

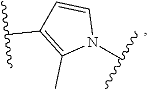 [SMILES: *c1ccn(*)c1C]

optionally substituted

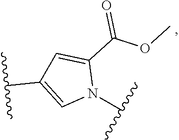 [SMILES: *c1cc(C(=O)OC)n(*)c1]

optionally substituted

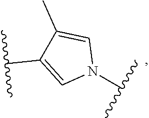 [SMILES: *c1cn(*)cc1C]

optionally substituted

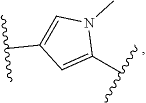 [SMILES: *c1cc(*)n(C)c1]

optionally substituted

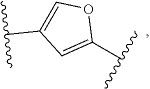 [SMILES: *c1coc(*)c1]

optionally substituted

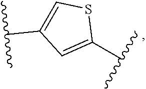 [SMILES: *c1csc(*)c1]

optionally substituted

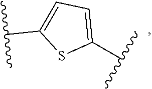 [SMILES: *c1ccc(*)s1]

optionally substituted

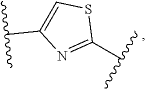 [SMILES: *c1csc(*)n1]

and optionally substituted S

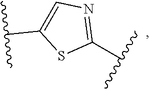 [SMILES: *c1cnc(*)s1]

W may be absent or W may be selected from the group consisting of the following structures that are optionally substituted: optionally substituted —CH₂—, optionally substituted —CH(CH₃)—, and optionally substituted

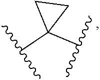 [SMILES: *C1(*)CC1]

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

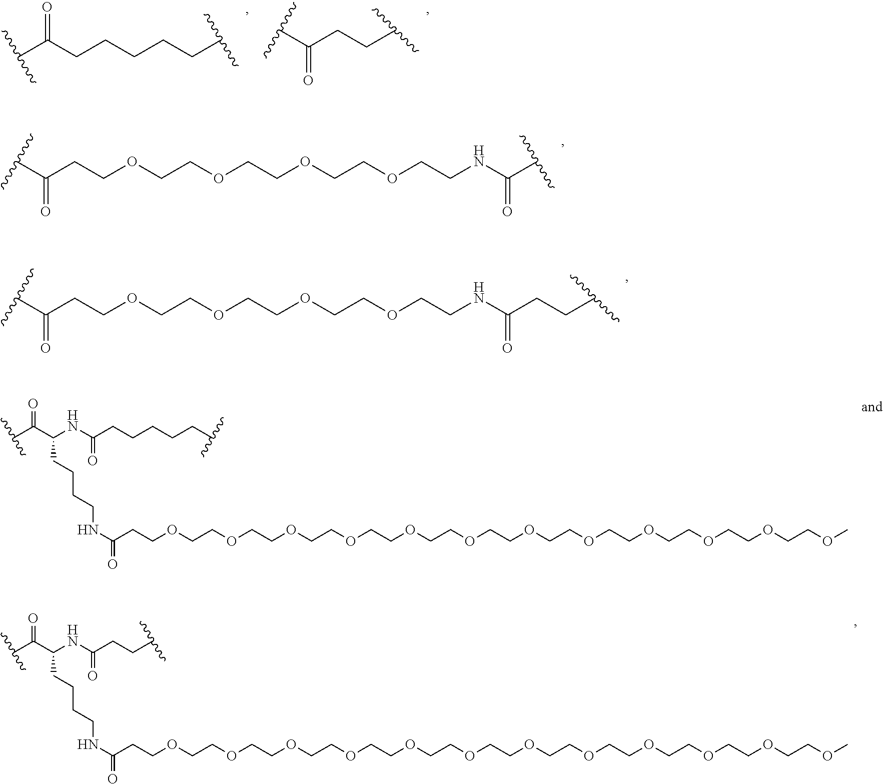 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L may be selected from the group consisting of:

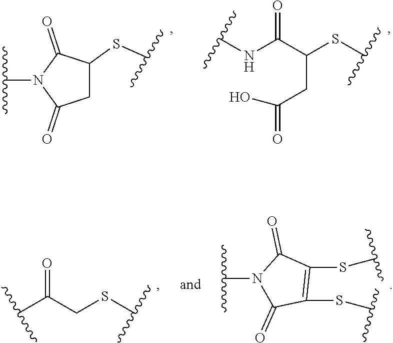 [SMILES: *SC1CC(=O)N(*)C1=O ; *NC(=O)C(CC(=O)O)S* ; *SCC(*)=O ; *SC1=C(S*)C(=O)N(*)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-B), formula (II-b2-B), formula (II-b3-B) or formula (II-b4-B):

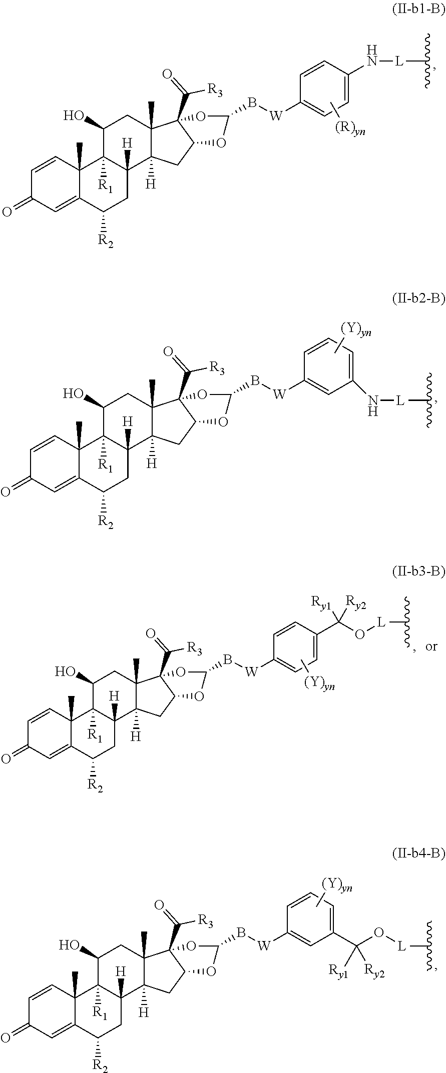

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen, halogen, and optionally substituted methyl, R₂ may be selected from the group consisting of: hydrogen, halogen, and optionally substituted methyl, R₃ may be selected from the group consisting of optionally substituted —CH₂Cl, optionally substituted —CH₂SH, optionally substituted —CH₂OH, optionally substituted

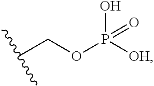 [SMILES: *COP(=O)(O)O]

optionally substituted

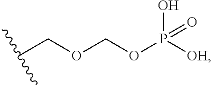 [SMILES: *COCOP(=O)(O)O]

optionally substituted

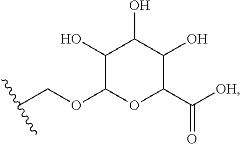 [SMILES: *COC1OC(C(=O)O)C(O)C(O)C1O]

optionally substituted

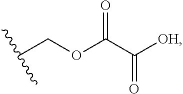 [SMILES: *COC(=O)C(=O)O]

optionally substituted

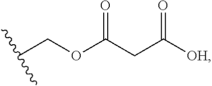 [SMILES: *COC(=O)CC(=O)O]

optionally substituted

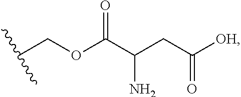 [SMILES: *COC(=O)C(N)CC(=O)O]

optionally substituted

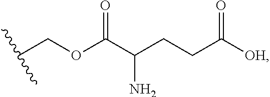 [SMILES: *COC(=O)C(N)CCC(=O)O]

optionally substituted

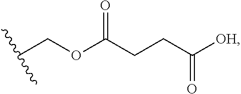 [SMILES: *COC(=O)CCC(=O)O]

optionally substituted

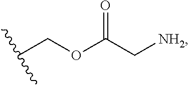 [SMILES: *COC(=O)CN]

optionally substituted

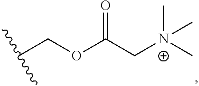 [SMILES: *COC(=O)C[N+](C)(C)C]

optionally substituted

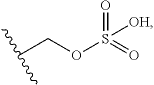 [SMILES: *COS(=O)(=O)O]

optionally substituted

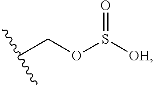 [SMILES: *COS(=O)O]

optionally substituted

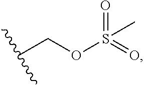 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

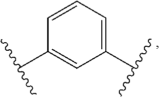 [SMILES: *c1cccc(*)c1]

optionally substituted

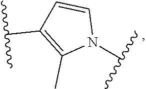 [SMILES: *c1ccn(*)c1C]

optionally substituted

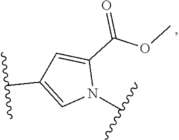 [SMILES: *c1cc(C(=O)OC)n(*)c1]

optionally substituted

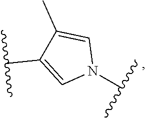 [SMILES: *c1cn(*)cc1C]

optionally substituted

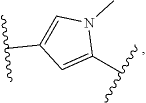 [SMILES: *c1cc(*)n(C)c1]

optionally substituted

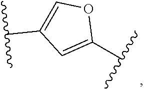 [SMILES: *c1coc(*)c1]

optionally substituted

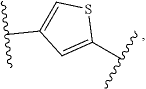 [SMILES: *c1csc(*)c1]

optionally substituted

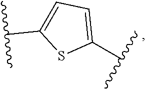 [SMILES: *c1ccc(*)s1]

optionally substituted

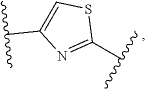 [SMILES: *c1csc(*)n1]

optionally substituted

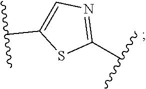 [SMILES: *c1cnc(*)s1]

W may be absent or W may be selected from the group consisting of the following structures that are optionally substituted:

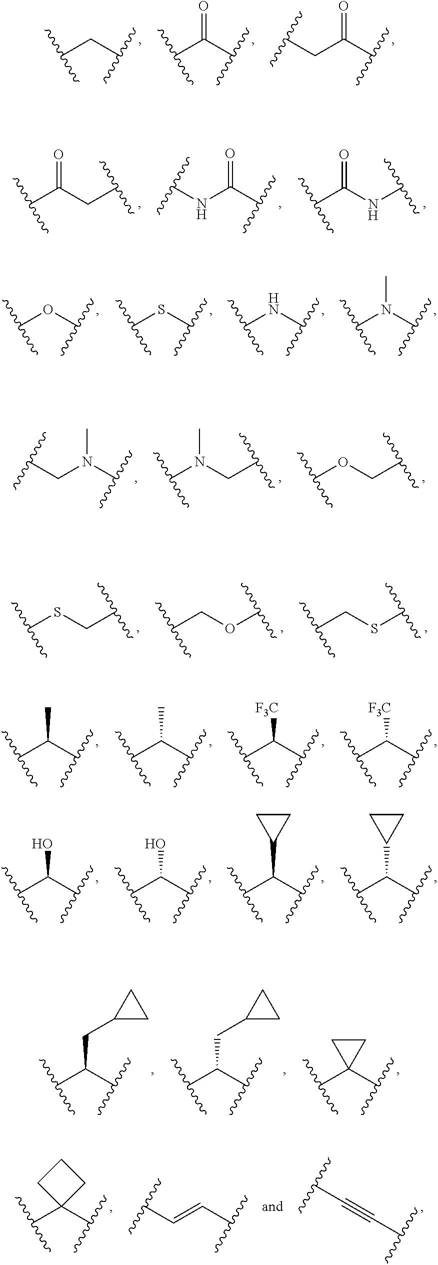

wherein when B may be optionally substituted

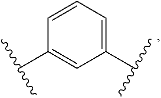 [SMILES: *c1cccc(*)c1]

### W may not be —S—;

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

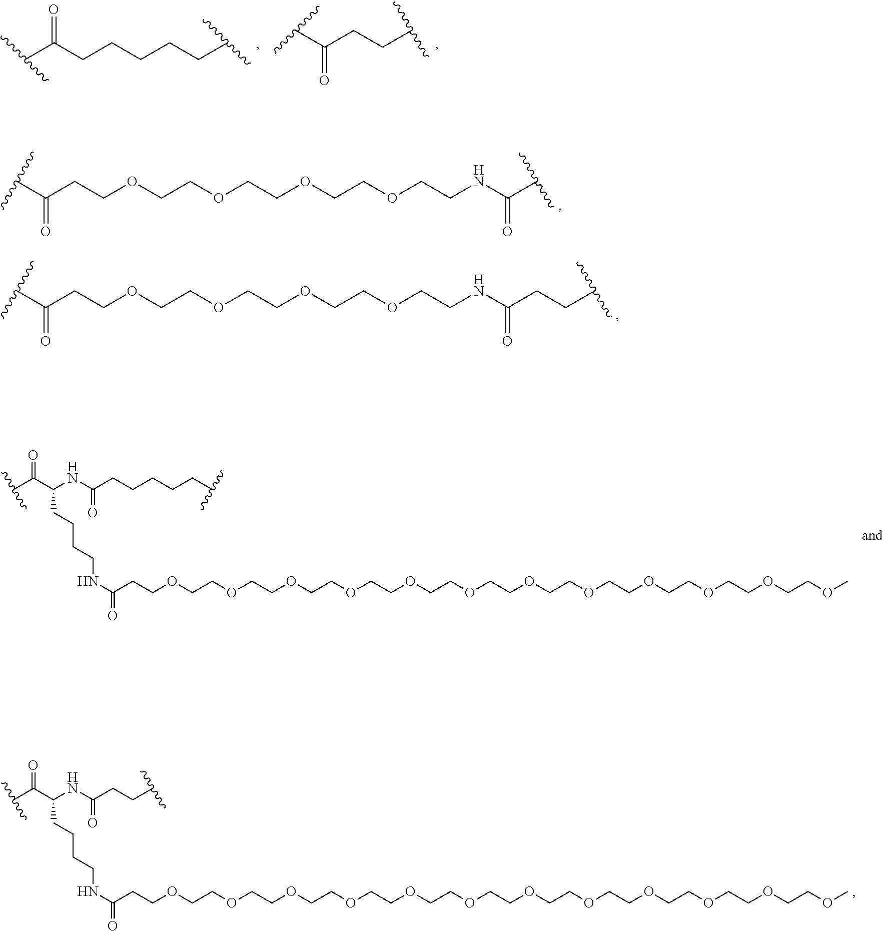 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

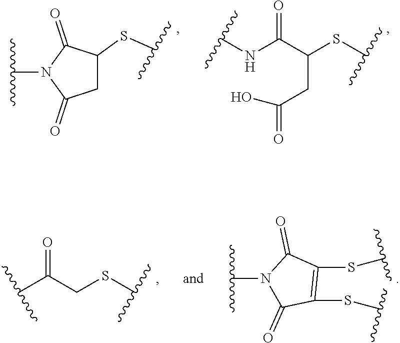 [SMILES: *SC1CC(=O)N(*)C1=O ; *NC(=O)C(CC(=O)O)S* ; *SCC(*)=O ; *SC1=C(S*)C(=O)N(*)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-B), formula (II-b2-B), formula (II-b3-B) or formula (II-b4-B):

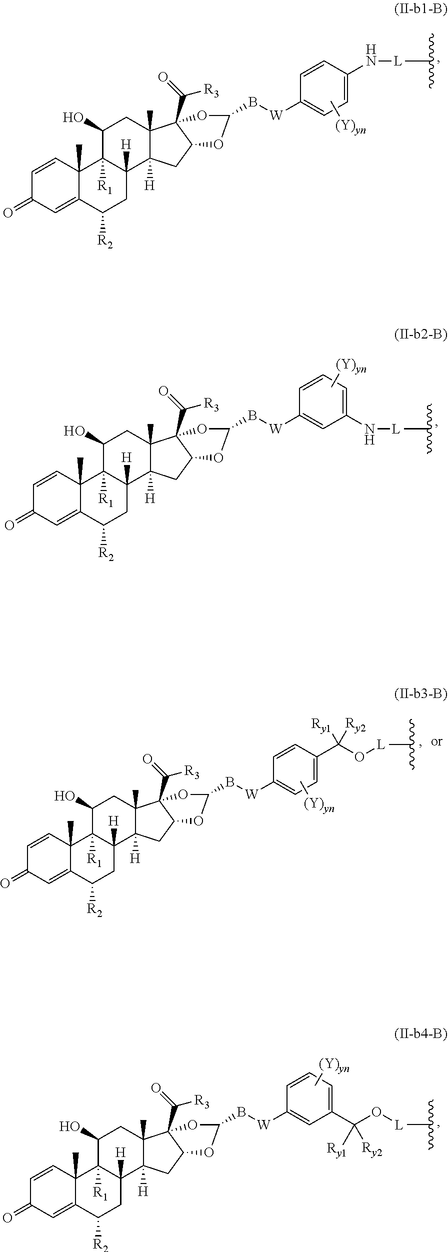

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen and halogen, R₂ may be selected from the group consisting of: hydrogen and halogen, R₃ may be selected from the group consisting of —CH₂OH, —SCH₂-halogen,

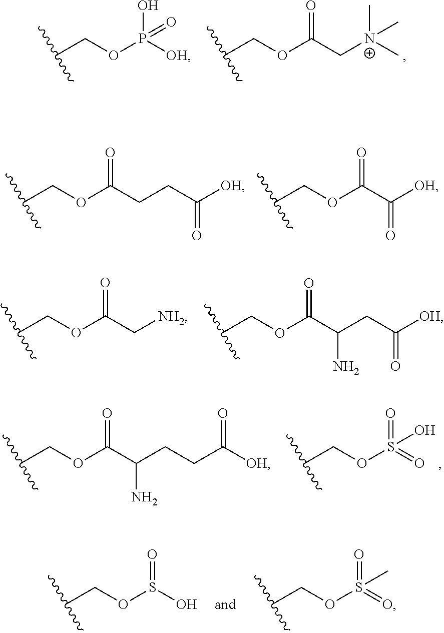

B may be selected from the group consisting of optionally substituted

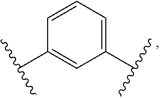 [SMILES: *c1cccc(*)c1]

optionally substituted

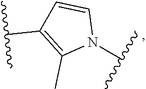 [SMILES: *c1ccn(*)c1C]

optionally substituted

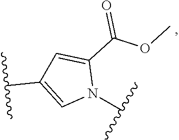 [SMILES: *c1cc(C(=O)OC)n(*)c1]

optionally substituted

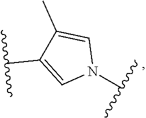 [SMILES: *c1cn(*)cc1C]

optionally substituted

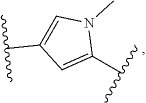 [SMILES: *c1cc(*)n(C)c1]

optionally substituted

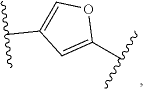 [SMILES: *c1coc(*)c1]

optionally substituted

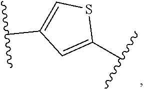 [SMILES: *c1csc(*)c1]

optionally substituted

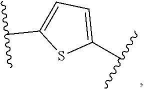 [SMILES: *c1ccc(*)s1]

optionally substituted

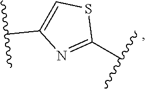 [SMILES: *c1csc(*)n1]

optionally substituted;

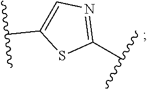 [SMILES: *c1cnc(*)s1]

W may be absent or W may be selected from the group consisting of the following structures that are optionally substituted: optionally substituted —CH₂—, optionally substituted —CH(CH₃)—, and optionally substituted

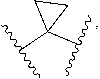 [SMILES: *C1(*)CC1]

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

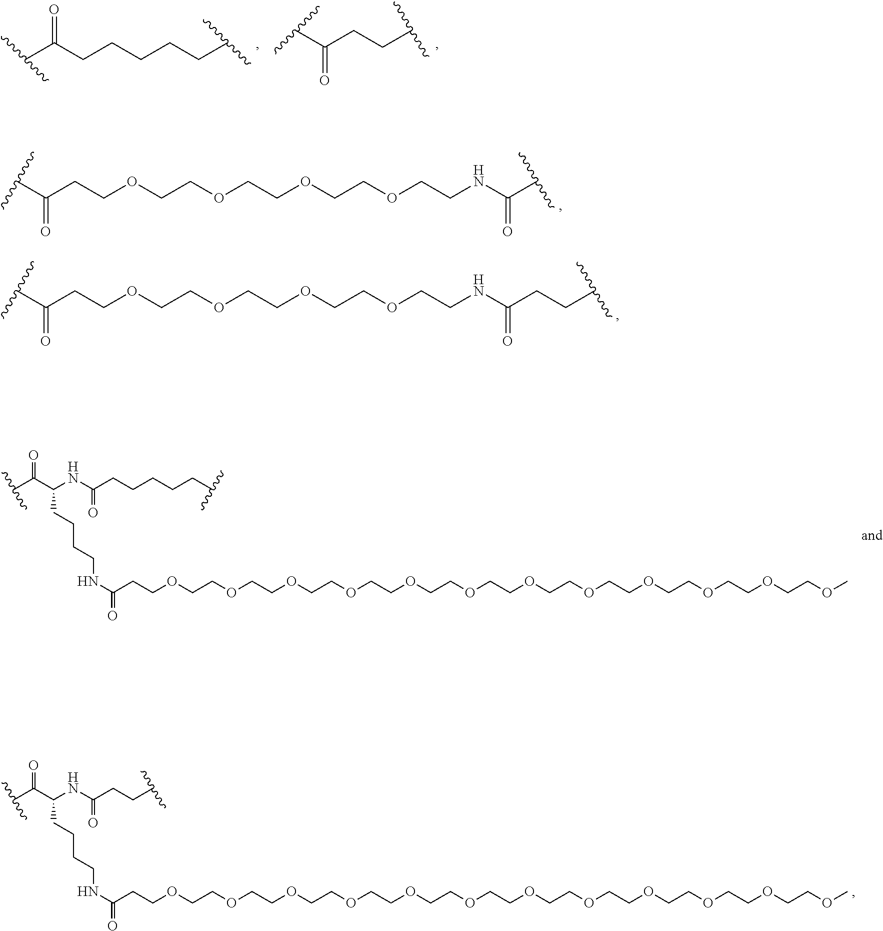 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L may be selected from the group consisting of:

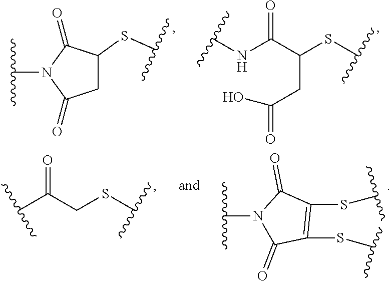

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-C), formula (I-a2-C), formula (I-a3-C) or formula (I-a4-C):

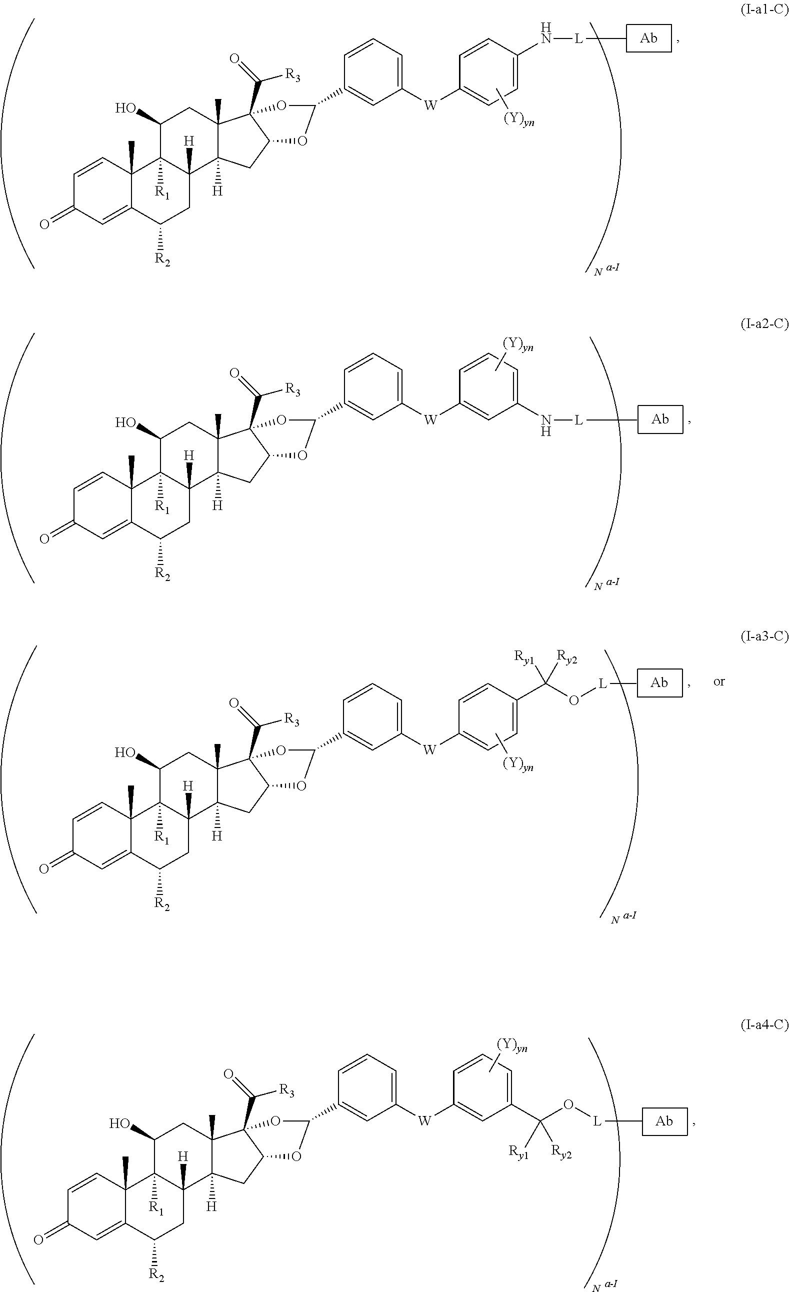

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen, halogen, and optionally substituted methyl, R₂ may be selected from the group consisting of hydrogen, halogen, and optionally substituted methyl, R₃ may be selected from the group consisting of optionally substituted —CH₂Cl, optionally substituted —CH₂SH, optionally substituted —CH₂OH, optionally substituted

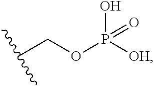 [SMILES: *COP(=O)(O)O]

optionally substituted

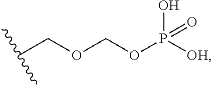 [SMILES: *COCOP(=O)(O)O]

optionally substituted

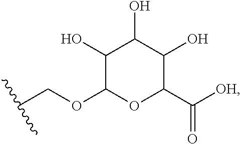 [SMILES: *COC1OC(C(=O)O)C(O)C(O)C1O]

optionally substituted

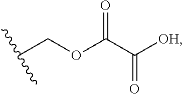 [SMILES: *COC(=O)C(=O)O]

optionally substituted

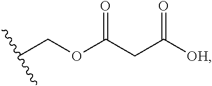 [SMILES: *COC(=O)CC(=O)O]

optionally substituted

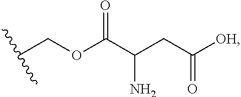 [SMILES: *COC(=O)C(N)CC(=O)O]

optionally substituted

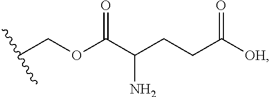 [SMILES: *COC(=O)C(N)CCC(=O)O]

optionally substituted

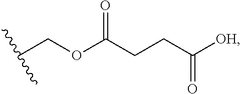 [SMILES: *COC(=O)CCC(=O)O]

optionally substituted

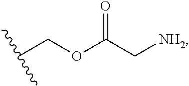 [SMILES: *COC(=O)CN]

optionally substituted

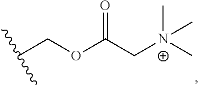 [SMILES: *COC(=O)C[N+](C)(C)C]

optionally substituted

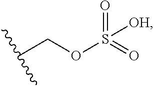 [SMILES: *COS(=O)(=O)O]

optionally substituted

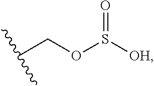 [SMILES: *COS(=O)O]

optionally substituted

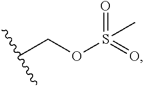 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
L may comprise -L³-L²-L¹-,
wherein L³ may be absent or selected from the group consisting of glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

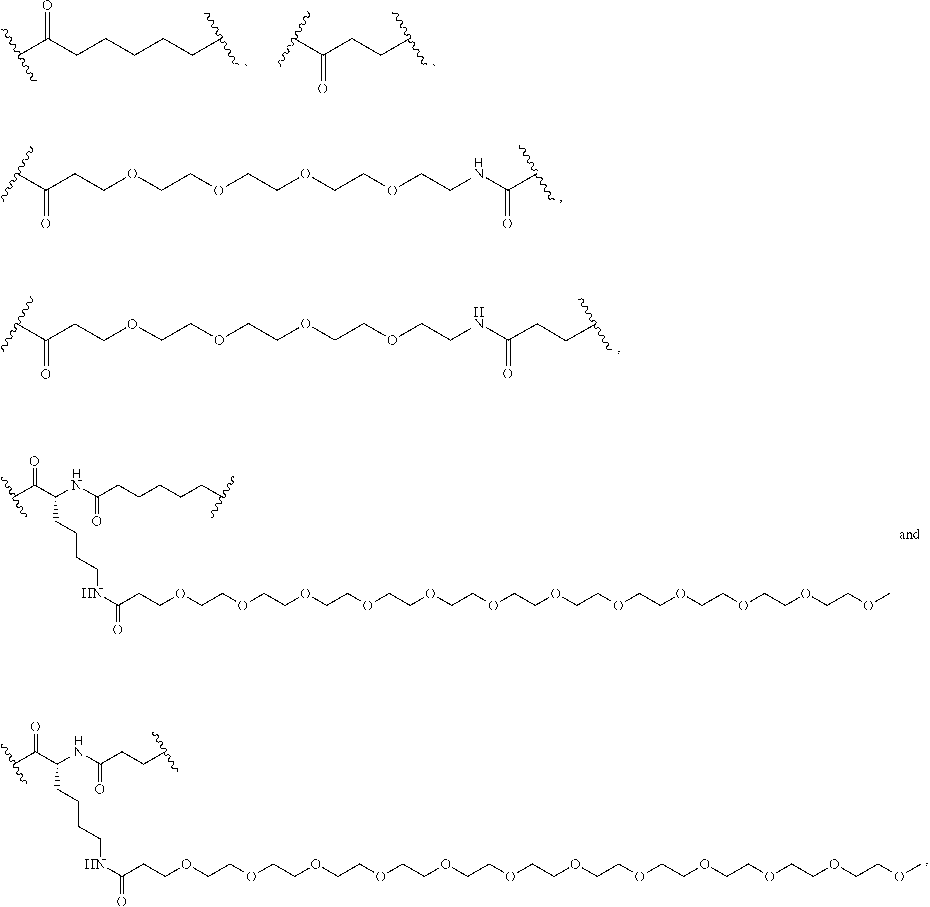 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

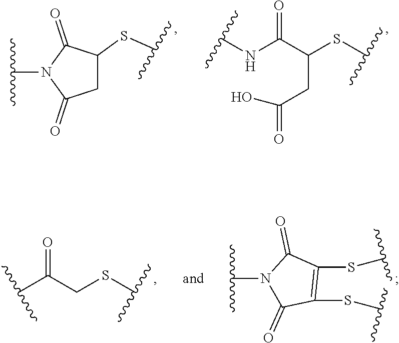 [SMILES: *SC1CC(=O)N(*)C1=O ; *NC(=O)C(CC(=O)O)S* ; *SCC(*)=O ; *SC1=C(S*)C(=O)N(*)C1=O]

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-C), formula (I-a2-C), formula (I-a3-C) or formula (I-a4-C):

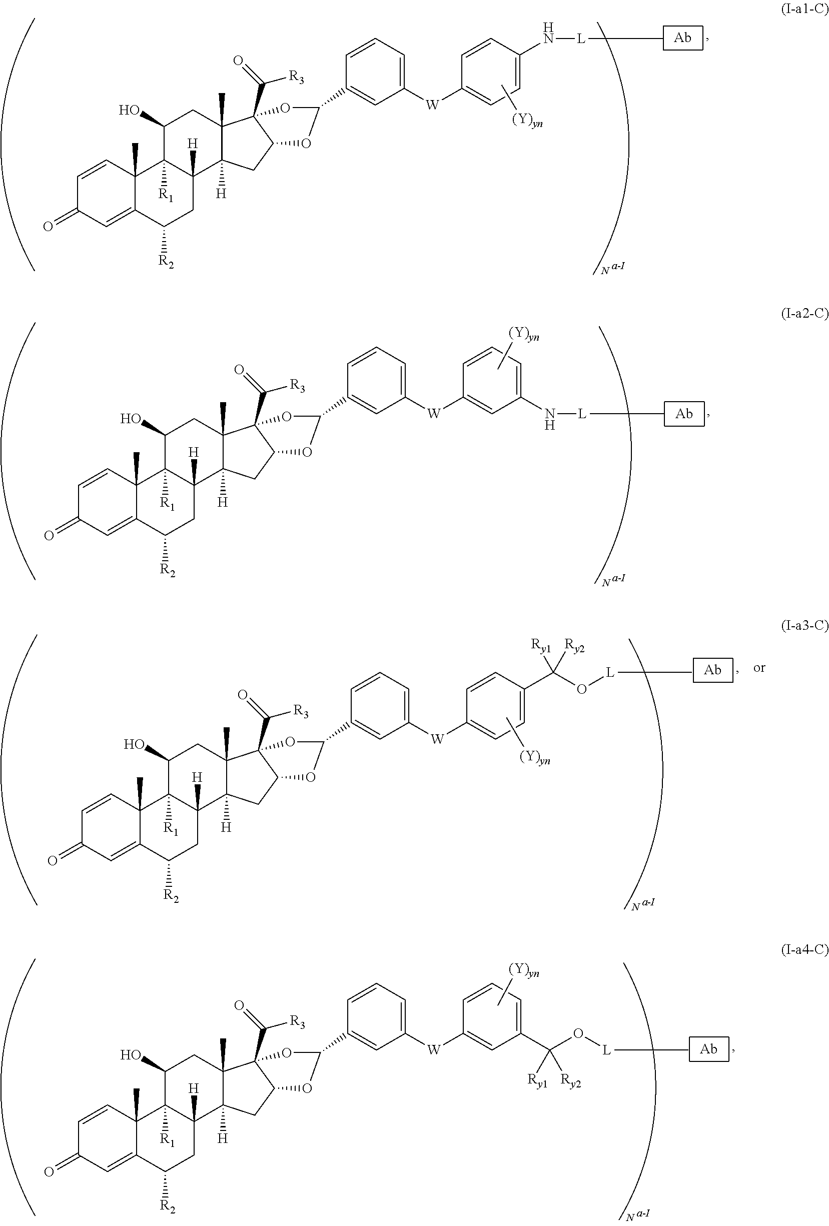

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂F,

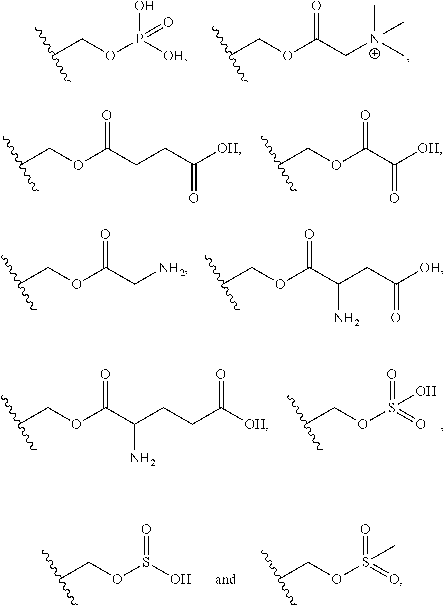

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),

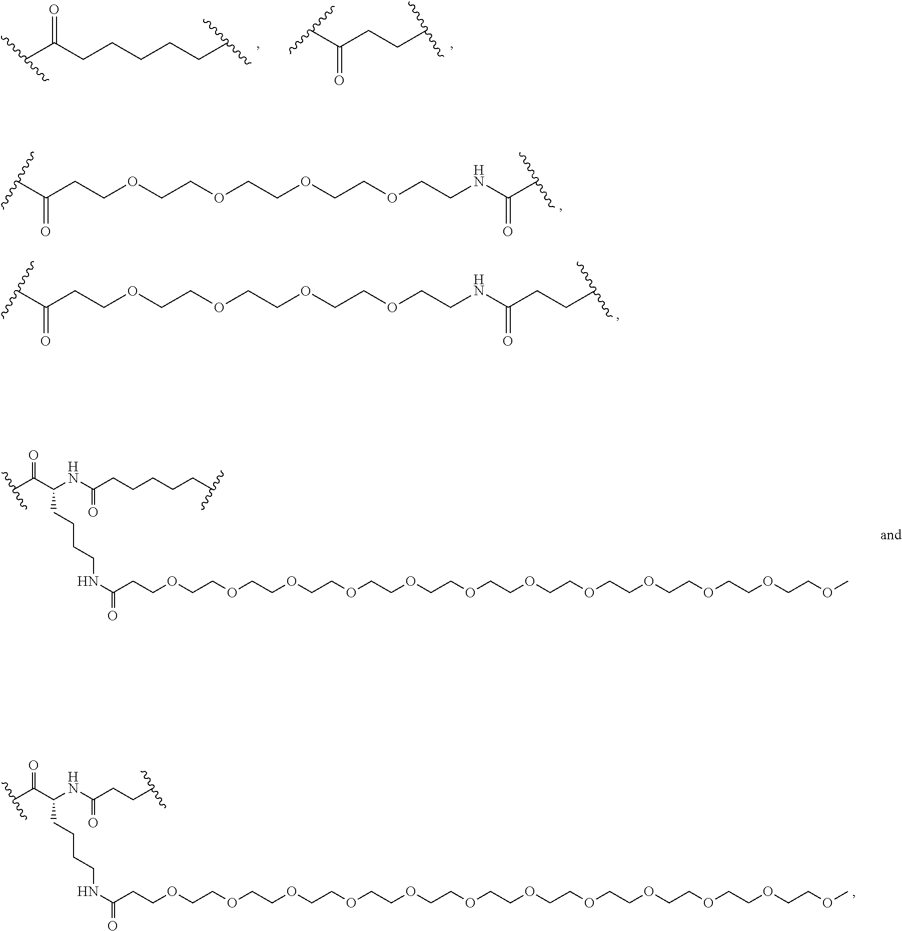 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

L² may be absent or selected from the group consisting of:
   and
L¹ may be selected from the group consisting of:

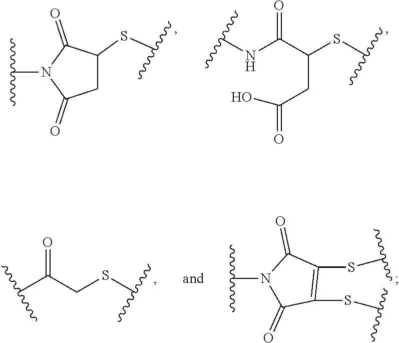 [SMILES: *SC1CC(=O)N(*)C1=O ; *NC(=O)C(CC(=O)O)S* ; *SCC(*)=O ; *SC1=C(S*)C(=O)N(*)C1=O]

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-C), formula (II-a2-C), formula (II-a3-C) or formula (II-a4-C):

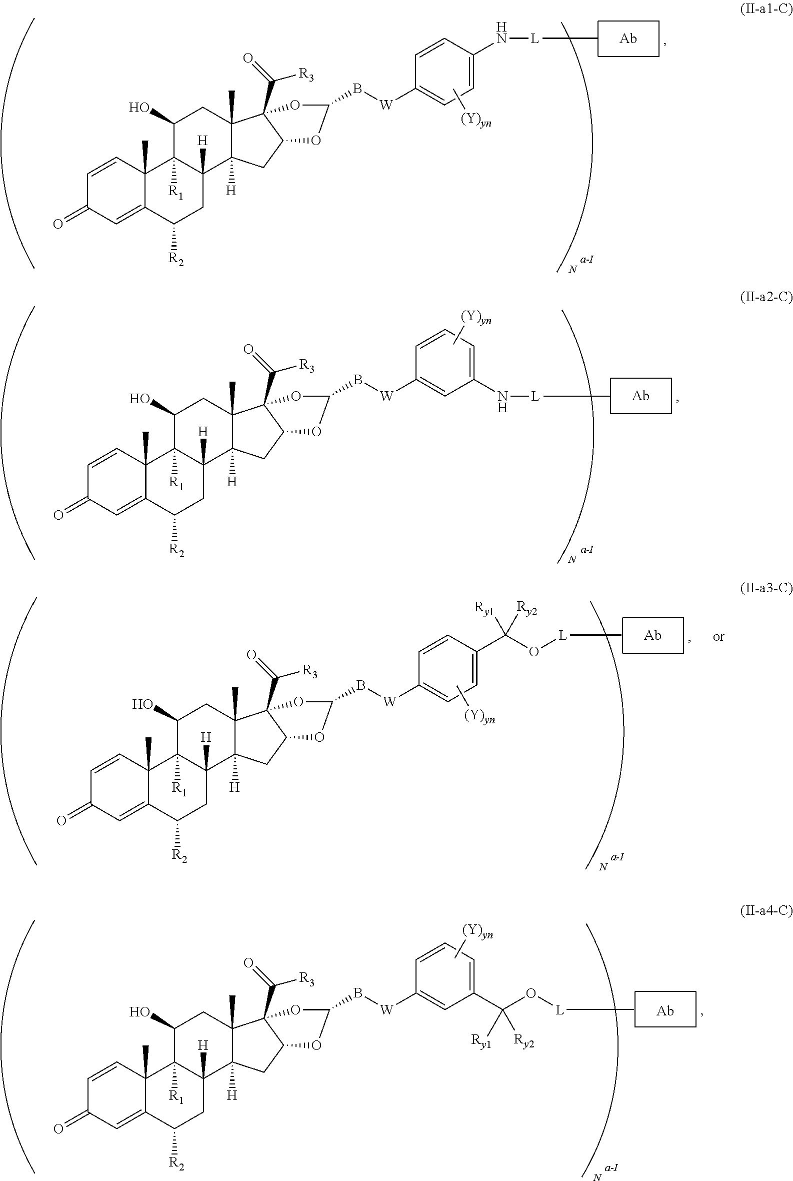

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

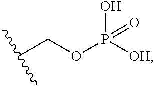 [SMILES: *COP(=O)(O)O]

optionally substituted

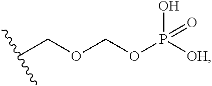 [SMILES: *COCOP(=O)(O)O]

optionally substituted

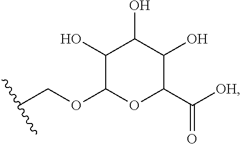 [SMILES: *COC1OC(C(=O)O)C(O)C(O)C1O]

optionally substituted

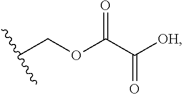 [SMILES: *COC(=O)C(=O)O]

optionally substituted

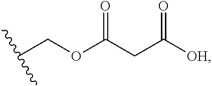 [SMILES: *COC(=O)CC(=O)O]

optionally substituted

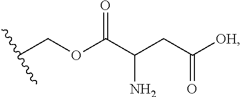 [SMILES: *COC(=O)C(N)CC(=O)O]

optionally substituted

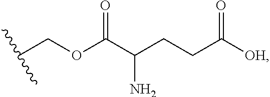 [SMILES: *COC(=O)C(N)CCC(=O)O]

optionally substituted

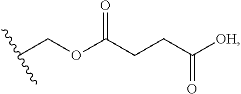 [SMILES: *COC(=O)CCC(=O)O]

optionally substituted

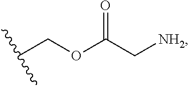 [SMILES: *COC(=O)CN]

optionally substituted

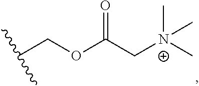 [SMILES: *COC(=O)C[N+](C)(C)C]

optionally substituted

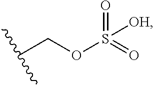 [SMILES: *COS(=O)(=O)O]

optionally substituted

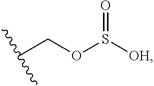 [SMILES: *COS(=O)O]

optionally substituted

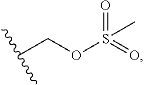 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

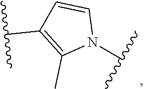 [SMILES: *c1ccn(*)c1C]

optionally substituted

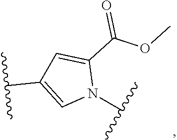 [SMILES: *c1cc(C(=O)OC)n(*)c1]

optionally substituted

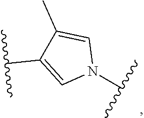 [SMILES: *c1cn(*)cc1C]

optionally substituted

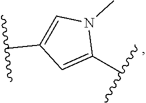 [SMILES: *c1cc(*)n(C)c1]

optionally substituted

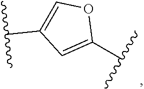 [SMILES: *c1coc(*)c1]

optionally substituted

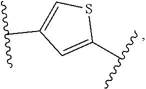 [SMILES: *c1csc(*)c1]

optionally substituted

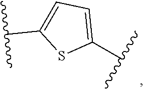 [SMILES: *c1ccc(*)s1]

optionally substituted

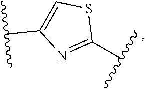 [SMILES: *c1csc(*)n1]

and optionally substituted

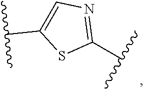 [SMILES: *c1cnc(*)s1]

W may be absent or W may be selected from the group consisting of the following structures that are optionally substituted:

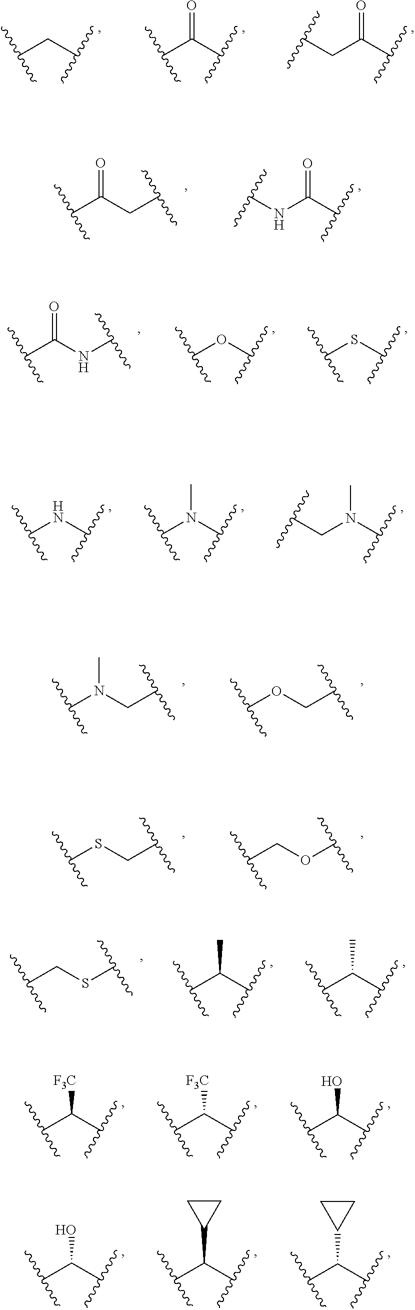

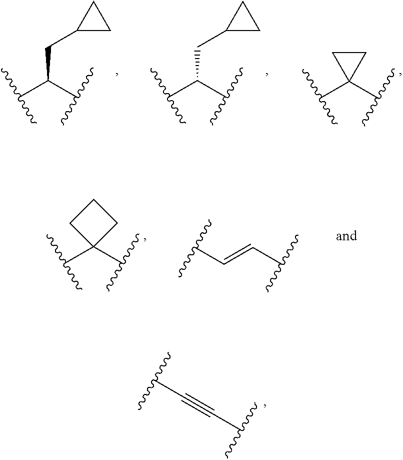

Y may be H and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

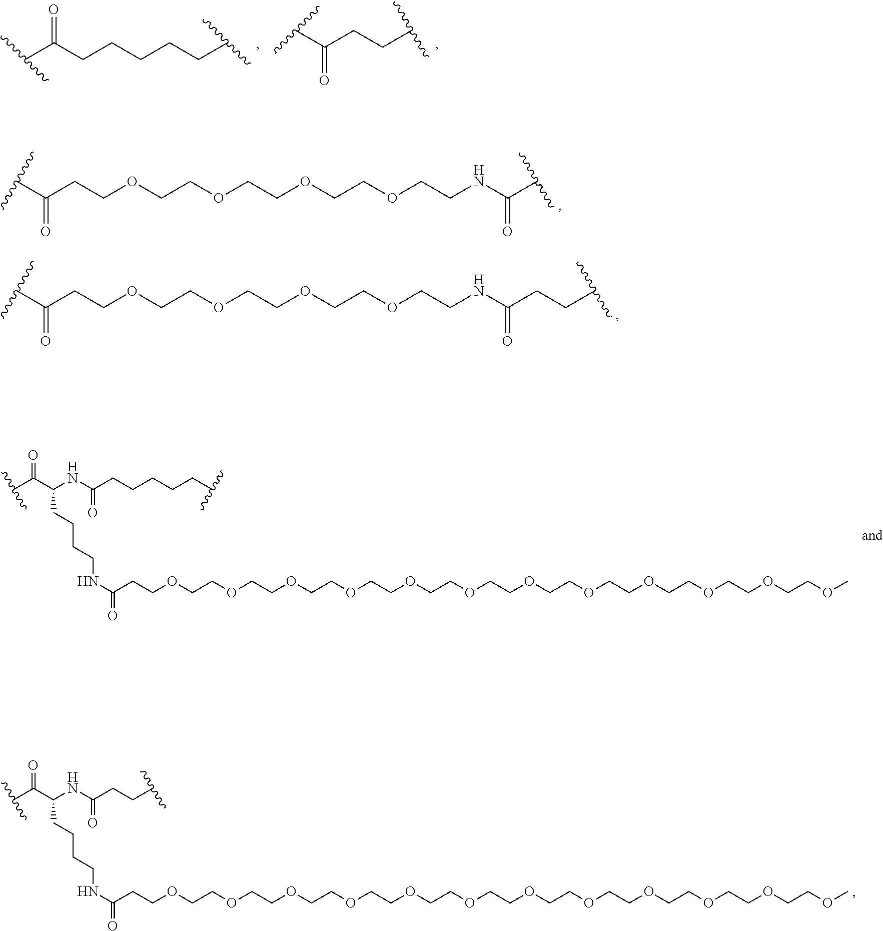 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

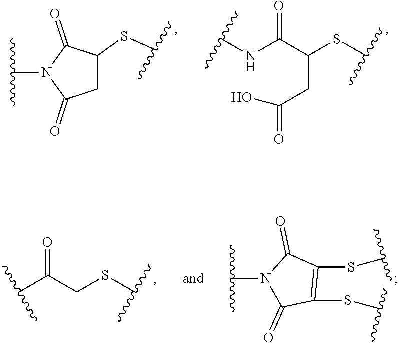 [SMILES: *SC1CC(=O)N(*)C1=O ; *NC(=O)C(CC(=O)O)S* ; *SCC(*)=O ; *SC1=C(S*)C(=O)N(*)C1=O]

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-C), formula (II-a2-C), formula (II-a3-C) or formula (II-a4-C):

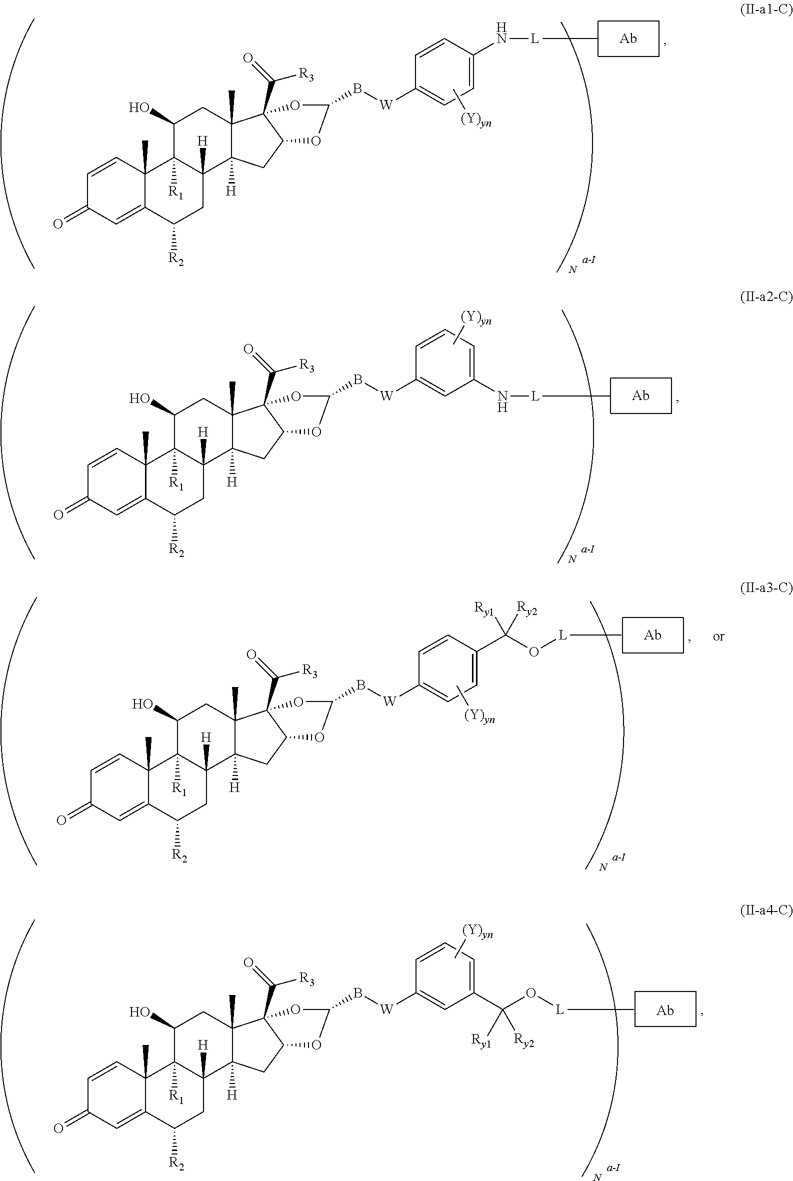

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂-halogen,

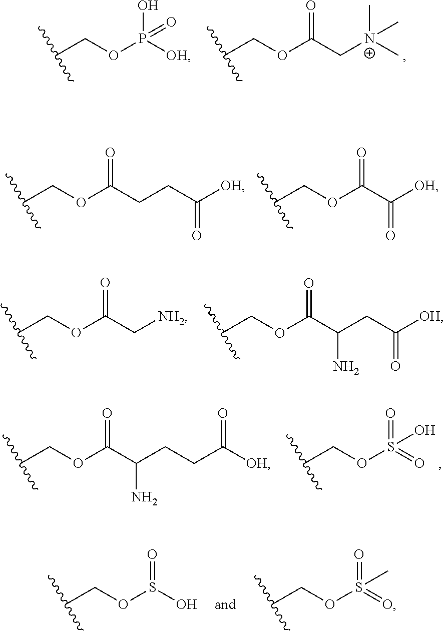

B may be selected from the group consisting of: optionally substituted

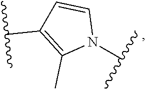 [SMILES: *c1ccn(*)c1C]

optionally substituted

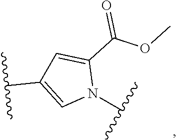 [SMILES: *c1cc(C(=O)OC)n(*)c1]

optionally substituted

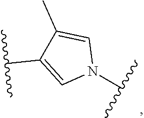 [SMILES: *c1cn(*)cc1C]

optionally substituted

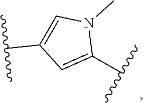 [SMILES: *c1cc(*)n(C)c1]

optionally substituted

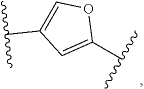 [SMILES: *c1coc(*)c1]

optionally substituted

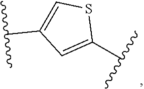 [SMILES: *c1csc(*)c1]

optionally substituted

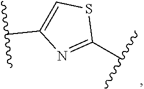 [SMILES: *c1csc(*)n1]

optionally substituted

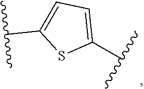 [SMILES: *c1ccc(*)s1]

and optionally substituted

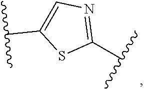 [SMILES: *c1cnc(*)s1]

W may be absent or W may be selected from the group consisting of the following structures that are optionally substituted: optionally substituted —CH₂—, optionally substituted —CH(CH₃)—, and optionally substituted

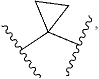 [SMILES: *C1(*)CC1]

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

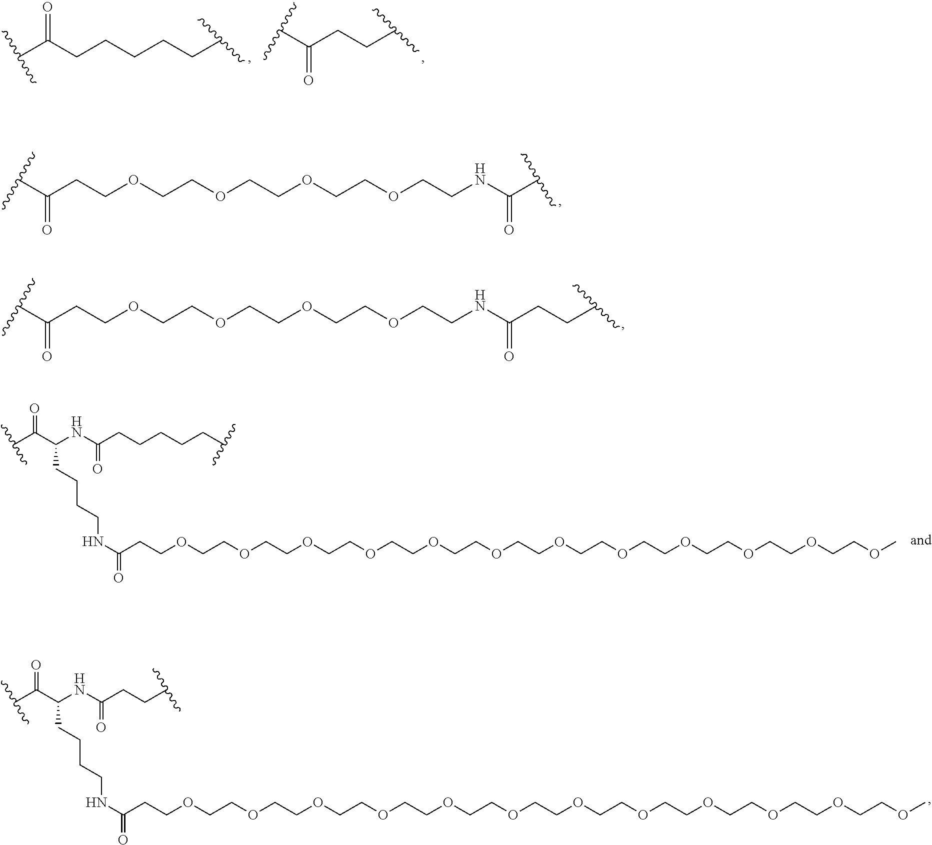 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

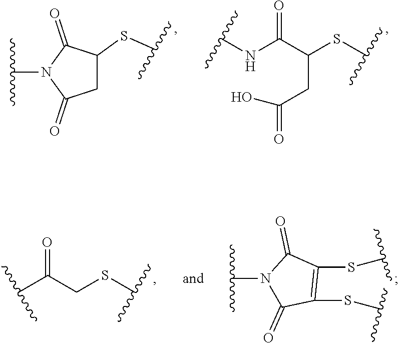 [SMILES: *SC1CC(=O)N(*)C1=O ; *NC(=O)C(CC(=O)O)S* ; *SCC(*)=O ; *SC1=C(S*)C(=O)N(*)C1=O]

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-C), formula (II-b2-C), formula (II-b3-C) or formula (II-b4-C):

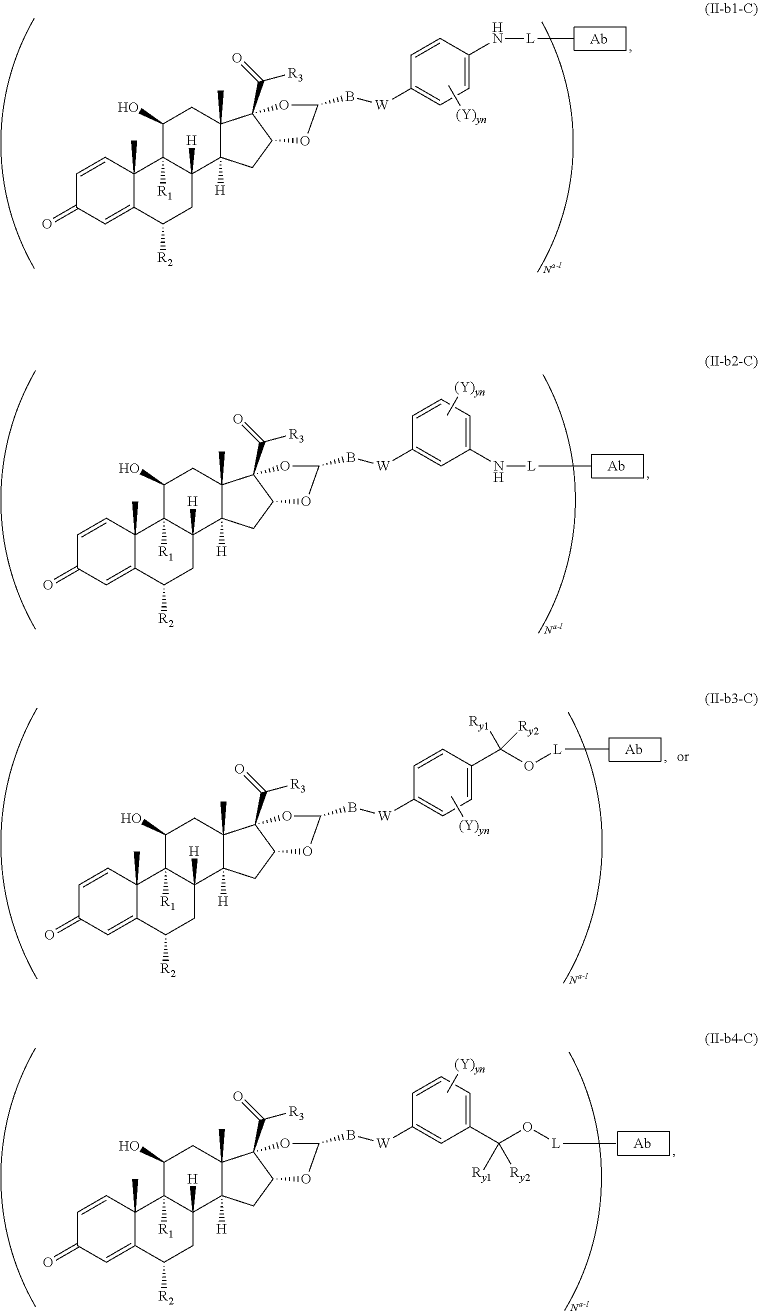

wherein Ry₁ may be selected from the group consisting of: hydrogen and optionally substituted methyl, Ry₂ may be selected from the group consisting of: hydrogen and optionally substituted methyl, R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

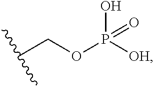 [SMILES: *COP(=O)(O)O]

optionally substituted

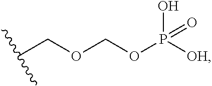 [SMILES: *COCOP(=O)(O)O]

optionally substituted

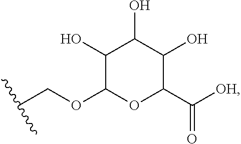 [SMILES: *COC1OC(C(=O)O)C(O)C(O)C1O]

optionally substituted

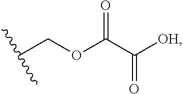 [SMILES: *COC(=O)C(=O)O]

optionally substituted

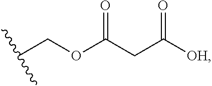 [SMILES: *COC(=O)CC(=O)O]

optionally substituted

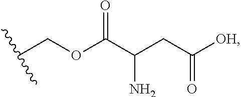 [SMILES: *COC(=O)C(N)CC(=O)O]

optionally substituted

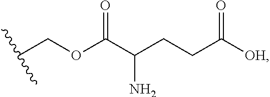 [SMILES: *COC(=O)C(N)CCC(=O)O]

optionally substituted

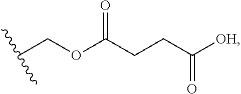 [SMILES: *COC(=O)CCC(=O)O]

optionally substituted

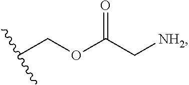 [SMILES: *COC(=O)CN]

optionally substituted

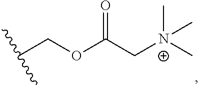 [SMILES: *COC(=O)C[N+](C)(C)C]

optionally substituted

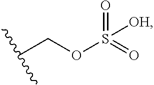 [SMILES: *COS(=O)(=O)O]

optionally substituted

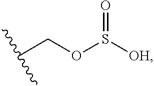 [SMILES: *COS(=O)O]

optionally substituted

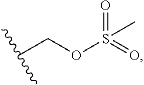 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of optionally substituted

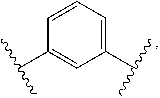 [SMILES: *c1cccc(*)c1]

optionally substituted

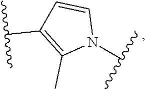 [SMILES: *c1ccn(*)c1C]

optionally substituted

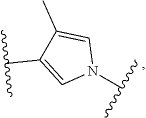 [SMILES: *c1cn(*)cc1C]

optionally substituted

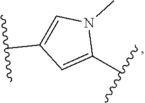 [SMILES: *c1cc(*)n(C)c1]

optionally substituted

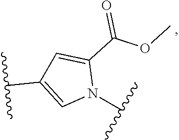 [SMILES: *c1cc(C(=O)OC)n(*)c1]

optionally substituted

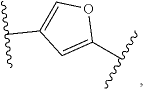 [SMILES: *c1coc(*)c1]

optionally substituted

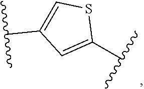 [SMILES: *c1csc(*)c1]

optionally substituted

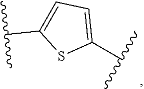 [SMILES: *c1ccc(*)s1]

optionally substituted

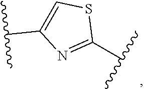 [SMILES: *c1csc(*)n1]

and optionally substituted;

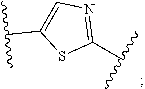 [SMILES: *c1cnc(*)s1]

W may be absent or W may be selected from the group consisting of the following structures that are optionally substituted:

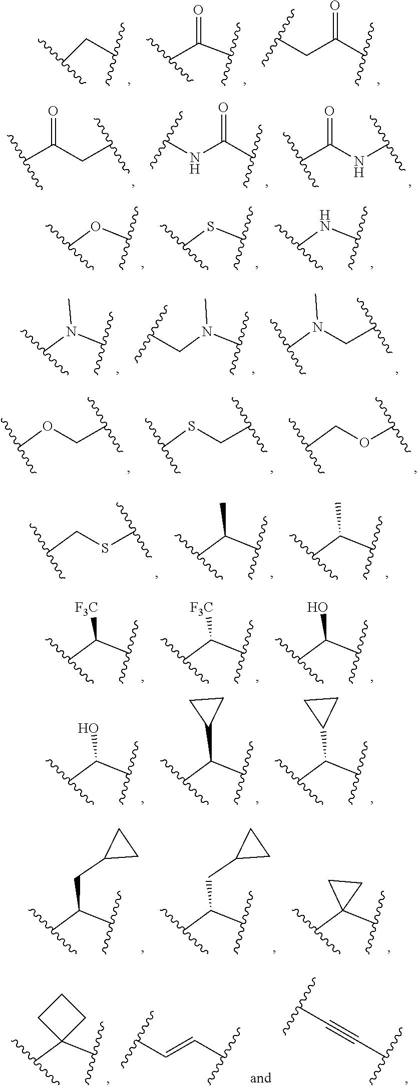

wherein when B may be optionally substituted

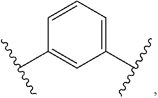 [SMILES: *c1cccc(*)c1]

### W may not be —S—;

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

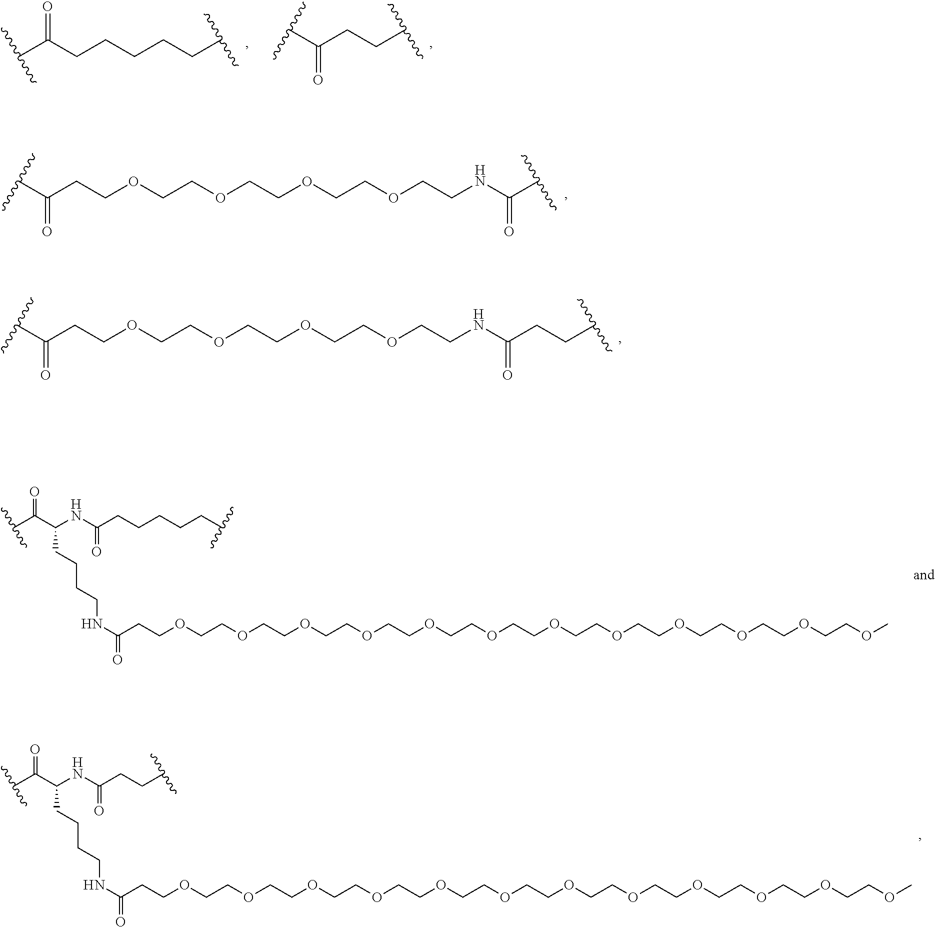 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

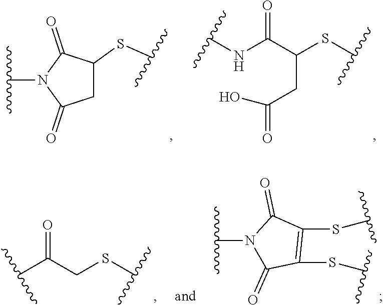

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-C), formula (II-b2-C), formula (II-b3-C) or formula (II-b4-C):

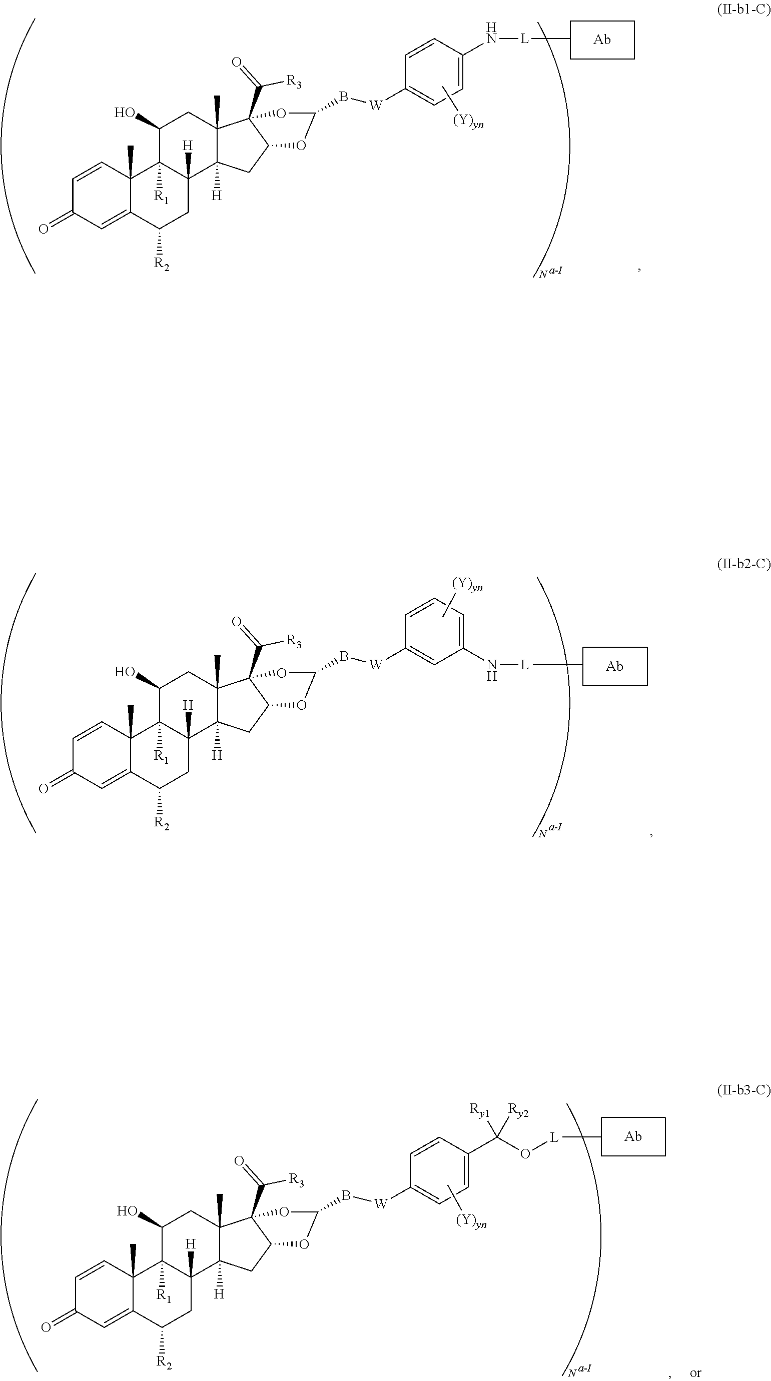

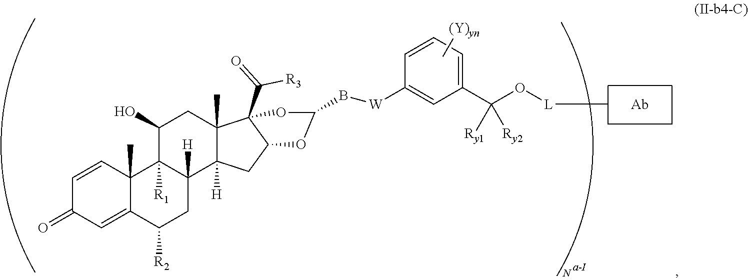

wherein Ry₁ may be selected from the group consisting of: hydrogen and optionally substituted methyl, Ry₂ may be selected from the group consisting of: hydrogen and optionally substituted methyl, R₁ may be selected from the group consisting of: hydrogen and halogen, R₂ may be selected from the group consisting of: hydrogen and halogen, R₃ may be selected from the group consisting of: —CH₂OH, —SCH₂-halogen,

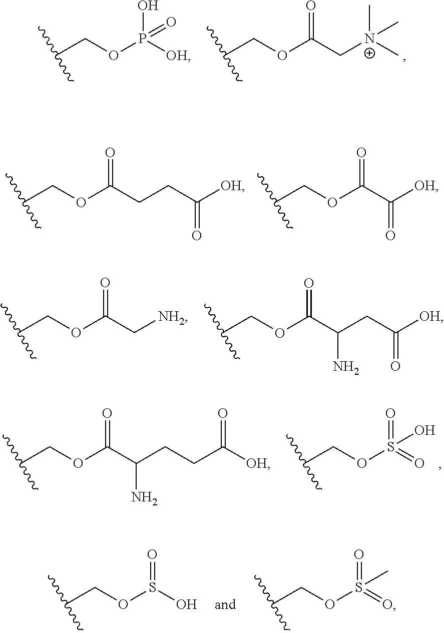

B may be selected from the group consisting of: optionally substituted

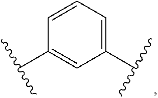 [SMILES: *c1cccc(*)c1]

optionally substituted

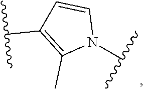 [SMILES: *c1ccn(*)c1C]

optionally substituted

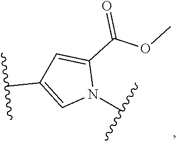 [SMILES: *c1cc(C(=O)OC)n(*)c1]

optionally substituted

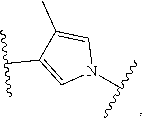 [SMILES: *c1cn(*)cc1C]

optionally substituted

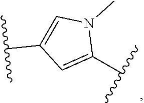 [SMILES: *c1cc(*)n(C)c1]

optionally substituted

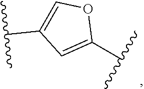 [SMILES: *c1coc(*)c1]

optionally substituted

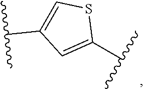 [SMILES: *c1csc(*)c1]

optionally substituted

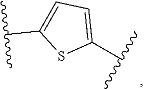 [SMILES: *c1ccc(*)s1]

optionally substituted

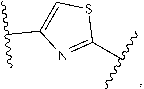 [SMILES: *c1csc(*)n1]

and optionally substituted S

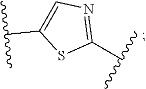 [SMILES: *c1cnc(*)s1]

W may be absent or W may be selected from the group consisting of the following structures that are optionally substituted: optionally substituted —CH₂—, optionally substituted —CH(CH₃)—, and optionally substituted

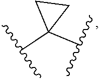 [SMILES: *C1(*)CC1]

Y may be H, and yn may be a number greater than 0;
L may comprise -L³-L²-L¹-,
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

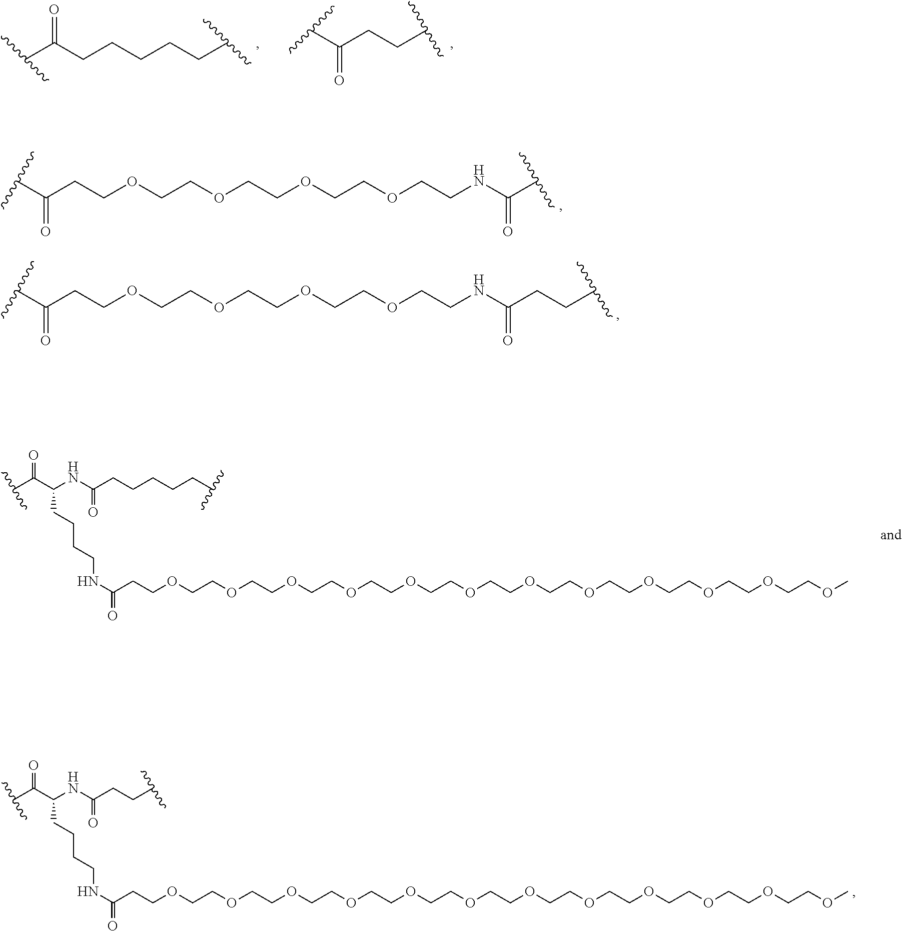 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L¹ may be selected from the group consisting of:

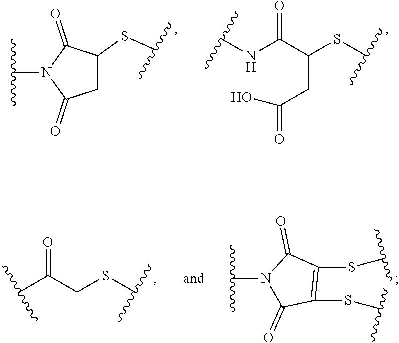 [SMILES: *SC1CC(=O)N(*)C1=O ; *NC(=O)C(CC(=O)O)S* ; *SCC(*)=O ; *SC1=C(S*)C(=O)N(*)C1=O]

Ab may be selected from the group consisting of: an anti-TNFα antibody or an antigen-binding fragment thereof, an anti-CD40 antibody or an antigen-binding fragment thereof, and an anti-IFNAR1 antibody or an antigen-binding fragment thereof; N^{a-1} may be a number from about 0 to about 20.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-D), formula (I-a2-D), formula (I-a3-D) or formula (I-a4-D):

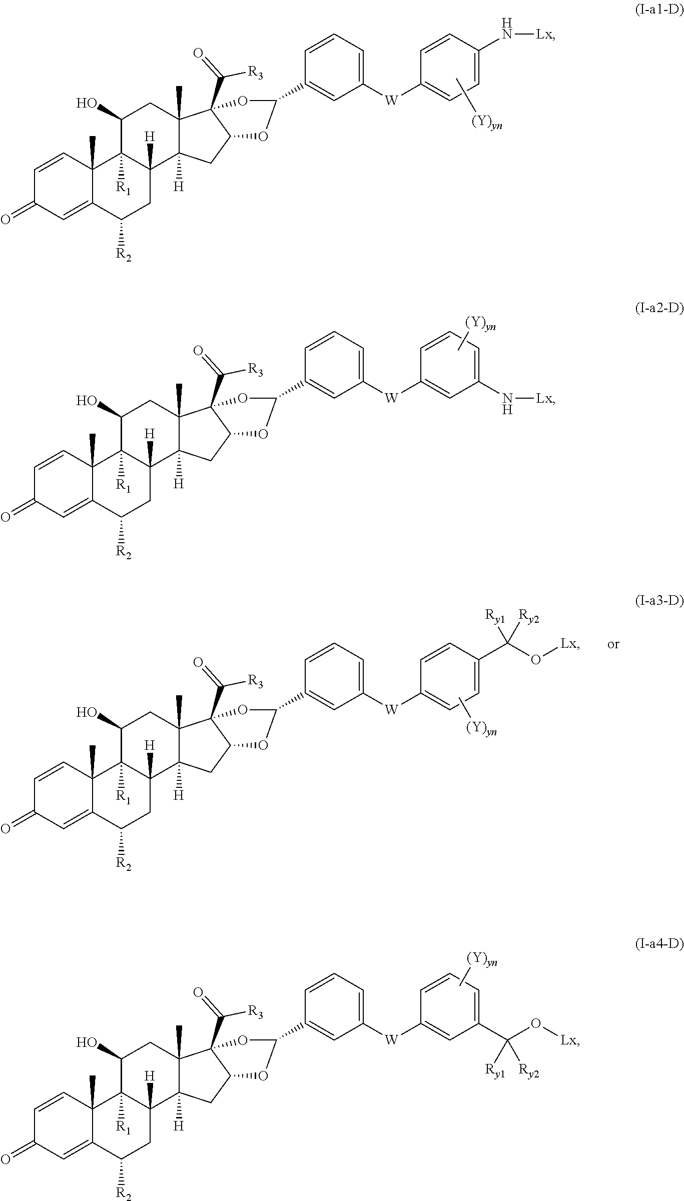

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

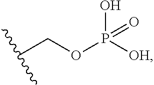 [SMILES: *COP(=O)(O)O]

optionally substituted

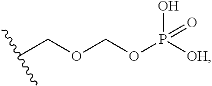 [SMILES: *COCOP(=O)(O)O]

optionally substituted

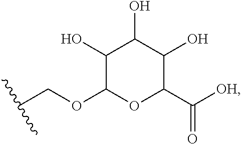 [SMILES: *COC1OC(C(=O)O)C(O)C(O)C1O]

optionally substituted

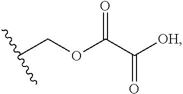 [SMILES: *COC(=O)C(=O)O]

optionally substituted

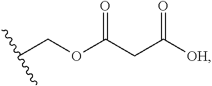 [SMILES: *COC(=O)CC(=O)O]

optionally substituted

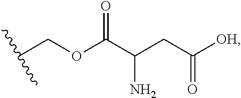 [SMILES: *COC(=O)C(N)CC(=O)O]

optionally substituted

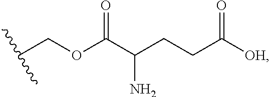 [SMILES: *COC(=O)C(N)CCC(=O)O]

optionally substituted

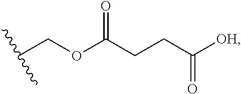 [SMILES: *COC(=O)CCC(=O)O]

optionally substituted

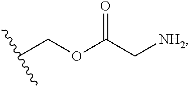 [SMILES: *COC(=O)CN]

optionally substituted

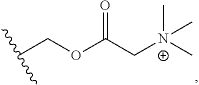 [SMILES: *COC(=O)C[N+](C)(C)C]

optionally substituted

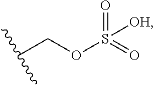 [SMILES: *COS(=O)(=O)O]

optionally substituted

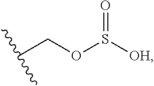 [SMILES: *COS(=O)O]

optionally substituted

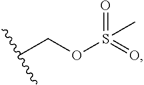 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Lx may comprise -L³-L²-L^{1x},
wherein L³ may be absent or selected from the group consisting of glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

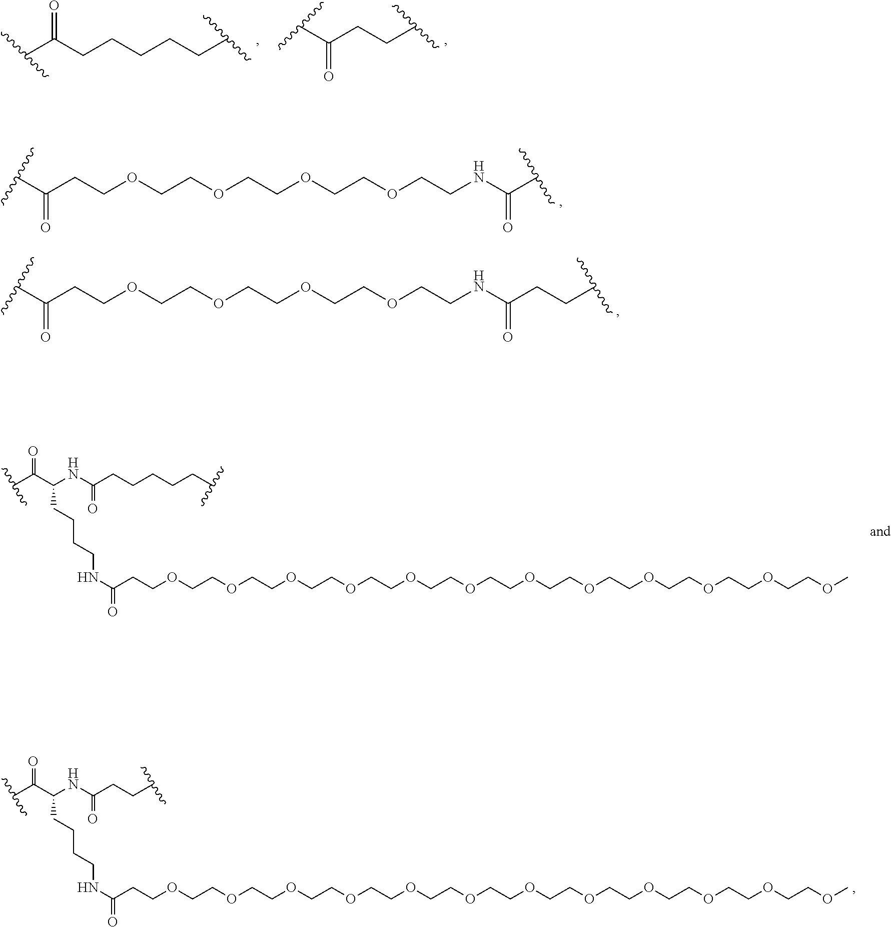 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L^{1x} may be selected from the group consisting of:

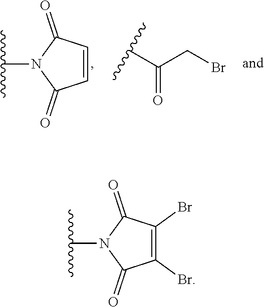

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-D), formula (I-a2-D), formula (I-a3-D) or formula (I-a4-D):

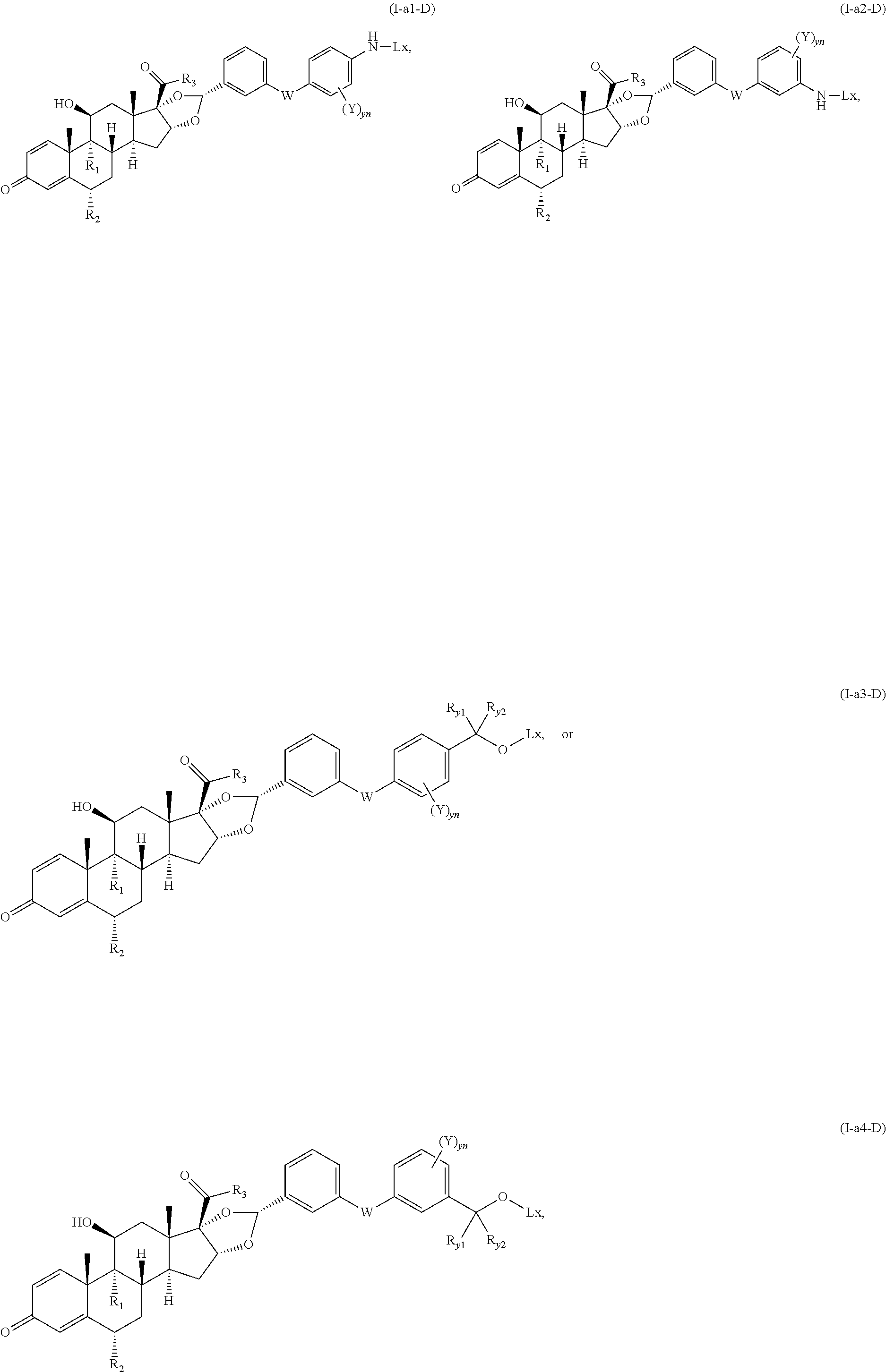

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group

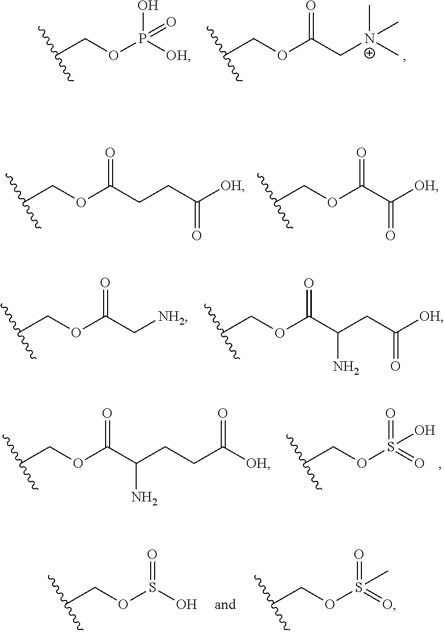

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Lx may comprise -L³-L²-L^{1x},
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

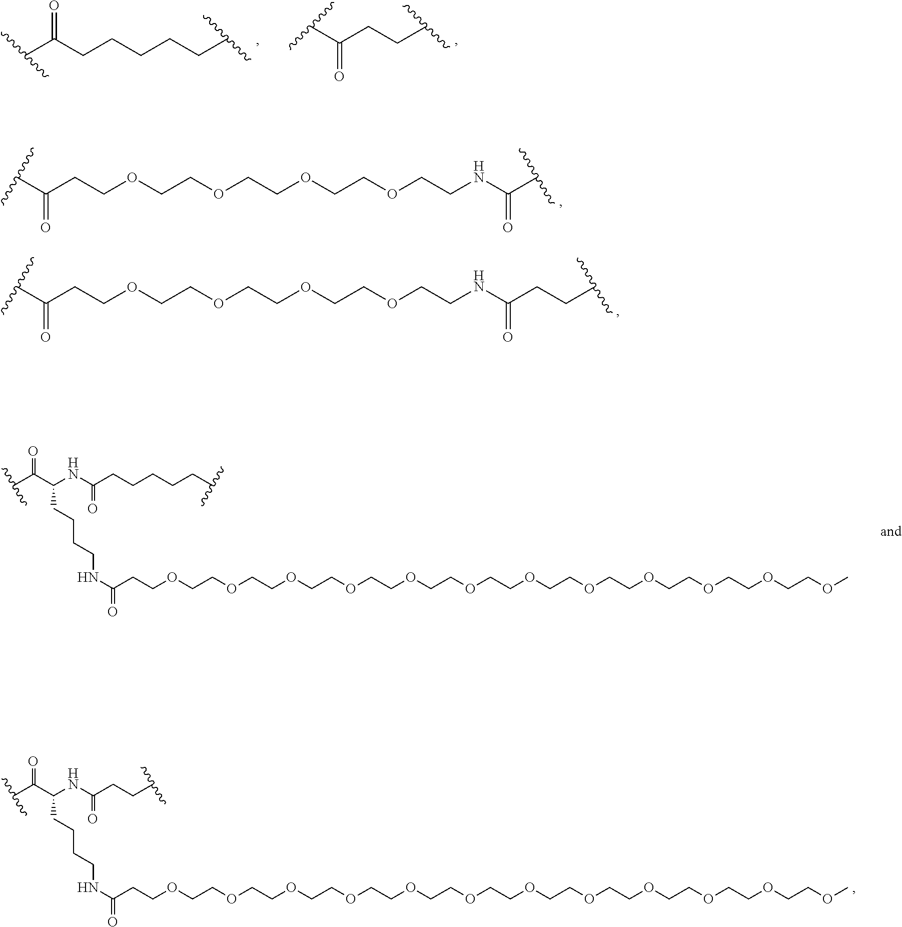 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L^{1x} may be selected from the group consisting of:

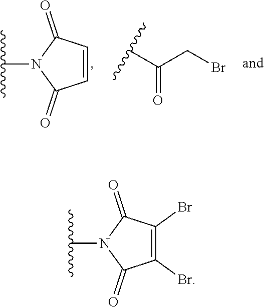

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-D), formula (II-a2-D), formula (II-a3-D) or formula (II-a4-D):

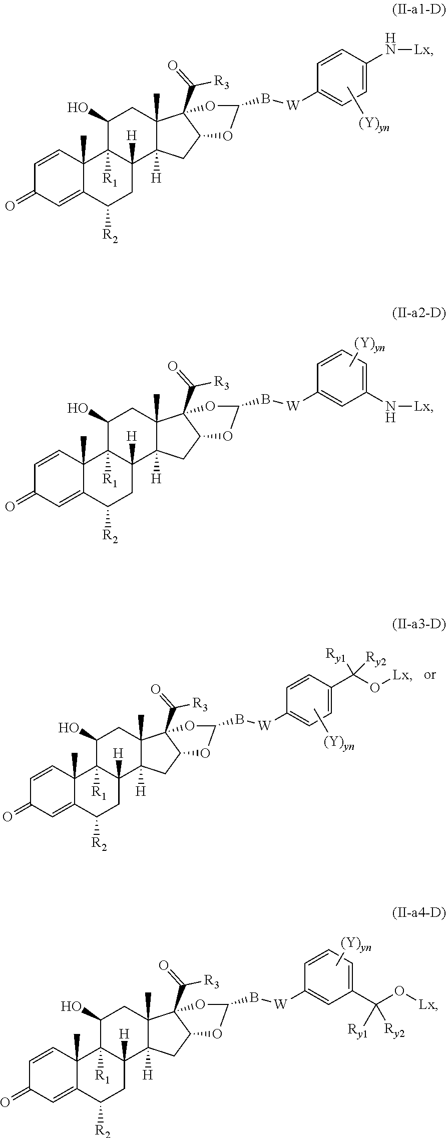

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

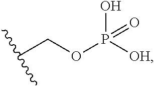 [SMILES: *COP(=O)(O)O]

optionally substituted

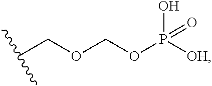 [SMILES: *COCOP(=O)(O)O]

optionally substituted

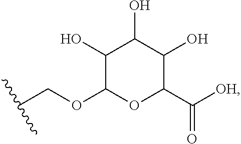 [SMILES: *COC1OC(C(=O)O)C(O)C(O)C1O]

optionally substituted

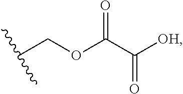 [SMILES: *COC(=O)C(=O)O]

optionally substituted

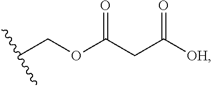 [SMILES: *COC(=O)CC(=O)O]

optionally substituted

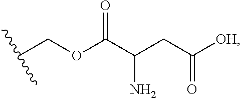 [SMILES: *COC(=O)C(N)CC(=O)O]

optionally substituted

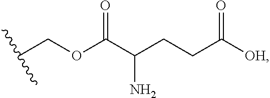 [SMILES: *COC(=O)C(N)CCC(=O)O]

optionally substituted

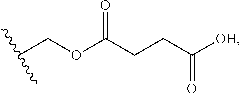 [SMILES: *COC(=O)CCC(=O)O]

optionally substituted

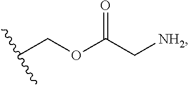 [SMILES: *COC(=O)CN]

optionally substituted

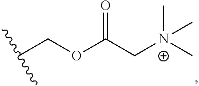 [SMILES: *COC(=O)C[N+](C)(C)C]

optionally substituted

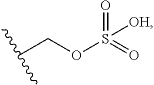 [SMILES: *COS(=O)(=O)O]

optionally substituted

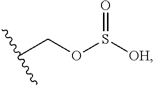 [SMILES: *COS(=O)O]

optionally substituted

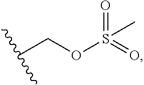 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

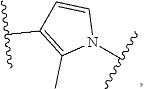 [SMILES: *c1ccn(*)c1C]

optionally substituted

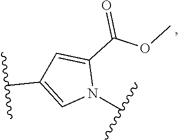 [SMILES: *c1cc(C(=O)OC)n(*)c1]

optionally substituted

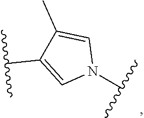 [SMILES: *c1cn(*)cc1C]

optionally substituted

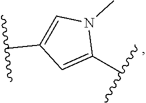 [SMILES: *c1cc(*)n(C)c1]

optionally substituted

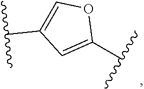 [SMILES: *c1coc(*)c1]

optionally substituted

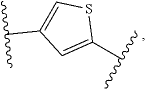 [SMILES: *c1csc(*)c1]

optionally substituted

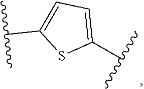 [SMILES: *c1ccc(*)s1]

optionally substituted

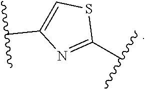 [SMILES: *c1csc(*)n1]

and optionally substituted

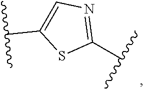 [SMILES: *c1cnc(*)s1]

W may be absent or W may be selected from the group consisting of the following structures that are optionally substituted:

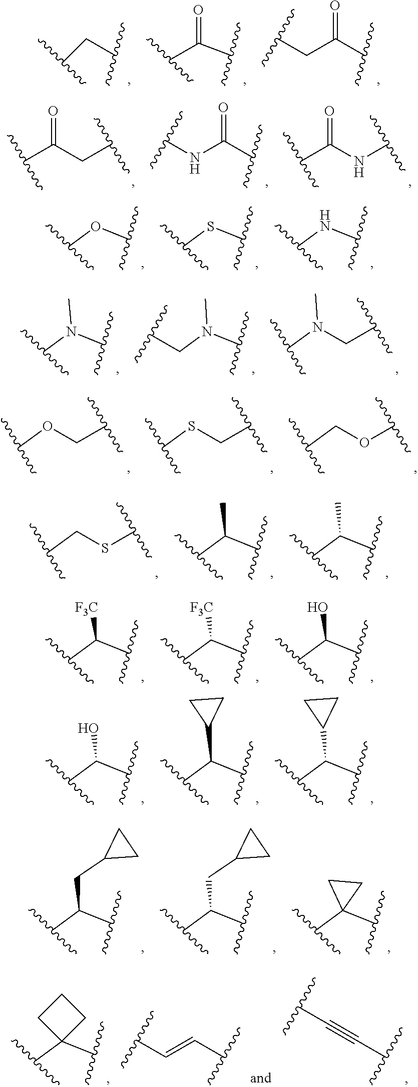

Y may be H, and yn may be a number greater than 0;
Lx may comprise -L³-L²-L^{1x},
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

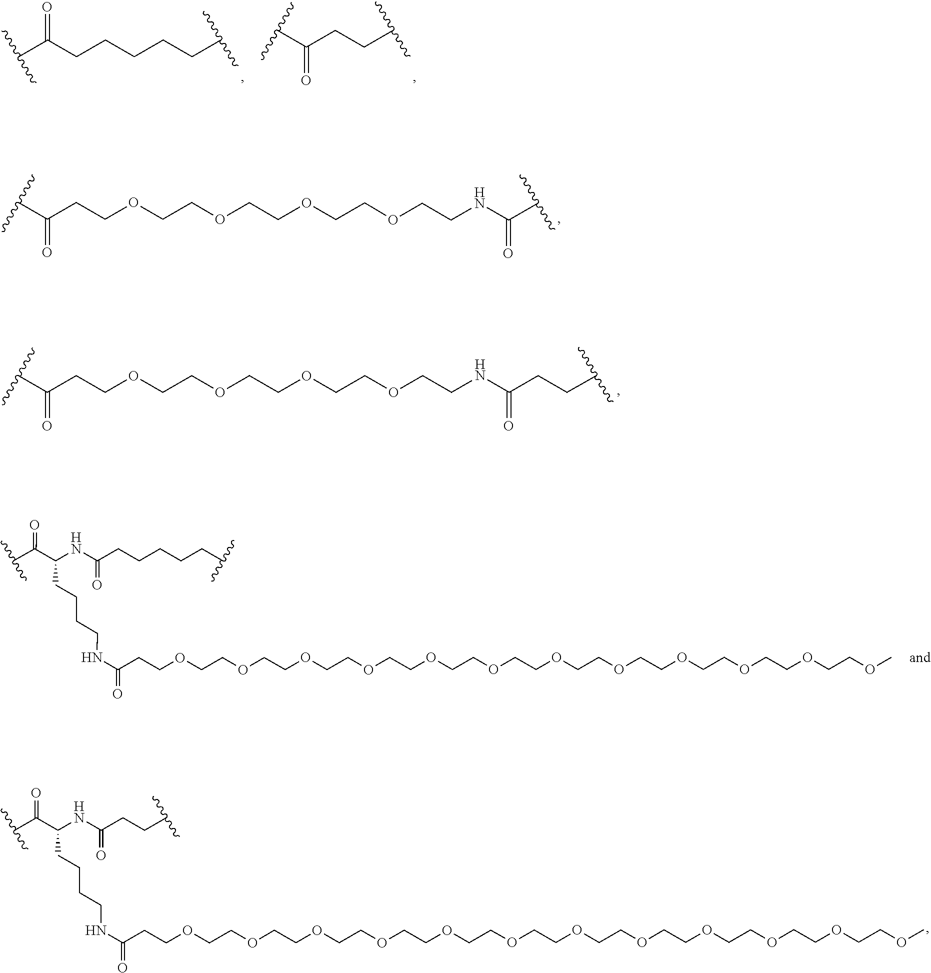 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and

L^{1x} may be selected from the group consisting of:

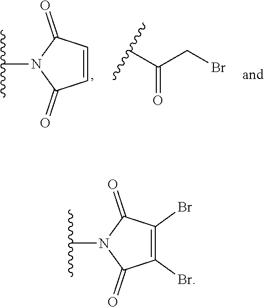

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-D), formula (II-a2-D), formula (II-a3-D) or formula (II-a4-D):

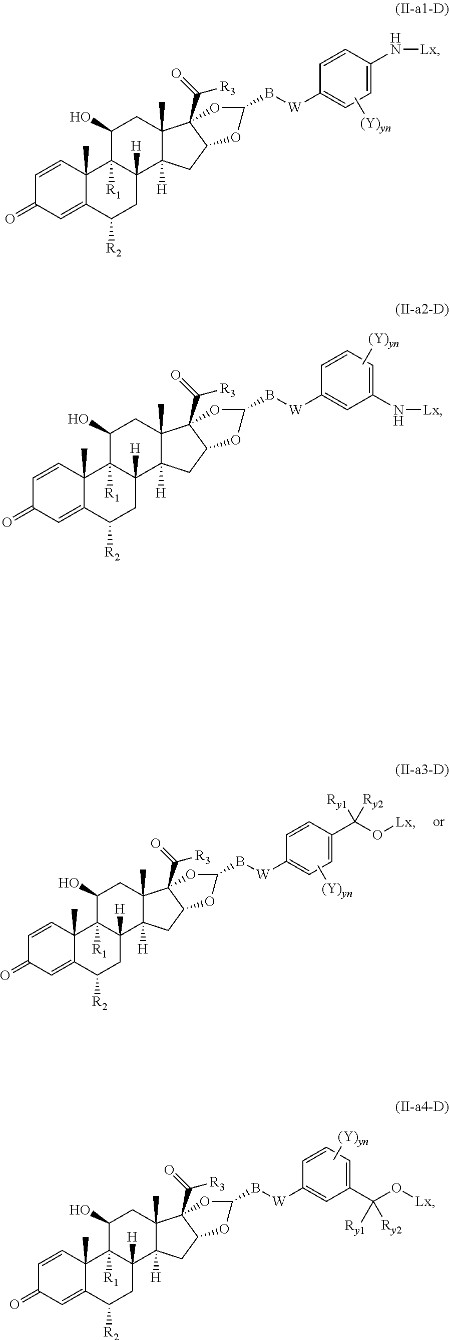

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂-halogen,

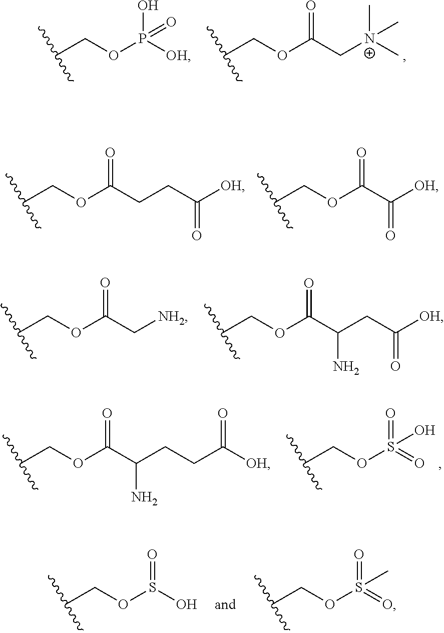

B may be selected from the group consisting of: optionally substituted

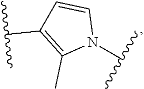 [SMILES: *c1ccn(*)c1C]

optionally substituted

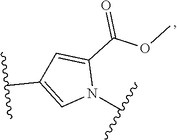 [SMILES: *c1cc(C(=O)OC)n(*)c1]

optionally substituted

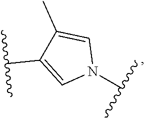 [SMILES: *c1cn(*)cc1C]

optionally substituted

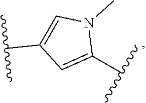 [SMILES: *c1cc(*)n(C)c1]

optionally substituted

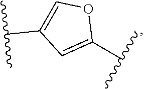 [SMILES: *c1coc(*)c1]

optionally substituted

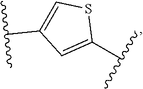 [SMILES: *c1csc(*)c1]

optionally substituted

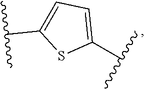 [SMILES: *c1ccc(*)s1]

optionally substituted

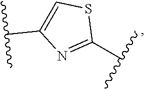 [SMILES: *c1csc(*)n1]

and optionally substituted

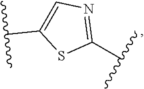 [SMILES: *c1cnc(*)s1]

W may be absent or W may be selected from the group consisting of the following structures that are optionally substituted: optionally substituted —CH₂—, optionally substituted —CH(CH₃)—, and optionally substituted

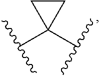 [SMILES: *C1(*)CC1]

Y may be H, and yn may be a number greater than 0;
Lx may comprise -L³-L²-L^{1x},
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

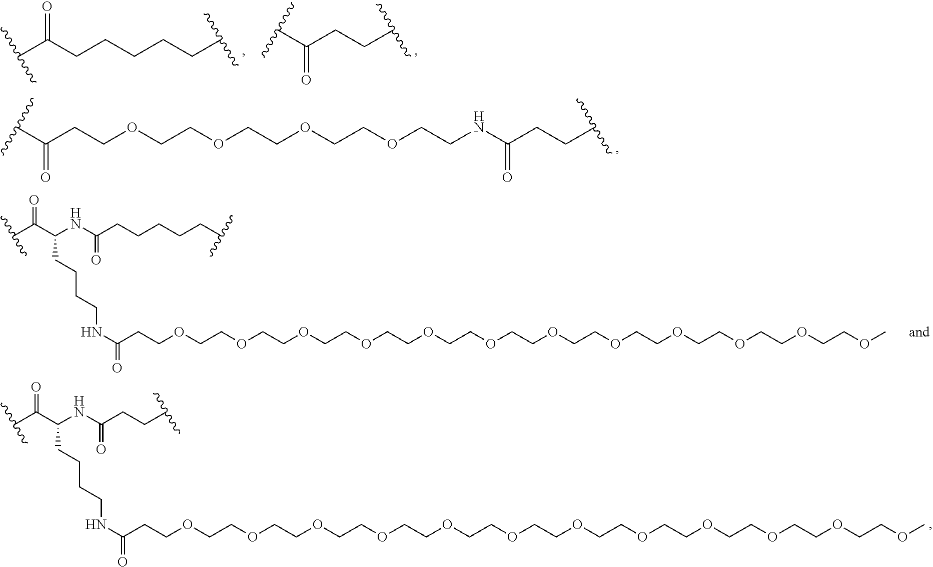

and
L^{1x} may be selected from the group consisting of:

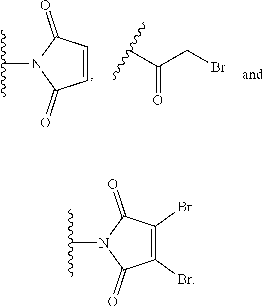

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-D), formula (II-b2-D), formula (II-b3-D) or formula (II-b4-D):

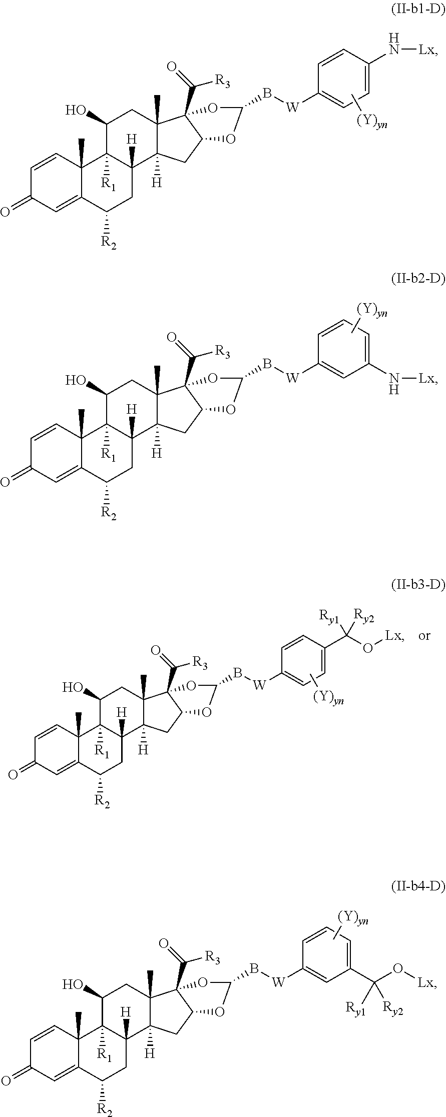

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

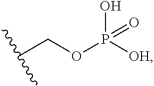 [SMILES: *COP(=O)(O)O]

optionally substituted

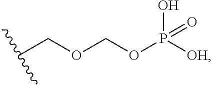 [SMILES: *COCOP(=O)(O)O]

optionally substituted

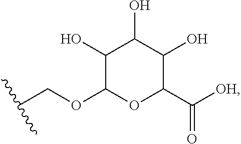 [SMILES: *COC1OC(C(=O)O)C(O)C(O)C1O]

optionally substituted

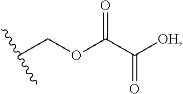 [SMILES: *COC(=O)C(=O)O]

optionally substituted

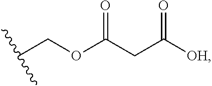 [SMILES: *COC(=O)CC(=O)O]

optionally substituted

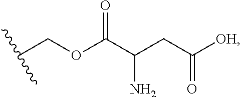 [SMILES: *COC(=O)C(N)CC(=O)O]

optionally substituted

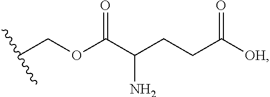 [SMILES: *COC(=O)C(N)CCC(=O)O]

optionally substituted

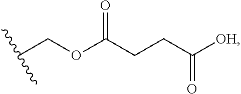 [SMILES: *COC(=O)CCC(=O)O]

optionally substituted

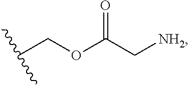 [SMILES: *COC(=O)CN]

optionally substituted

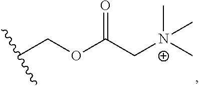 [SMILES: *COC(=O)C[N+](C)(C)C]

optionally substituted

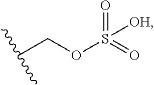 [SMILES: *COS(=O)(=O)O]

optionally substituted

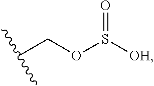 [SMILES: *COS(=O)O]

optionally substituted

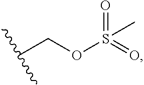 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

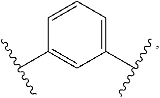 [SMILES: *c1cccc(*)c1]

optionally substituted

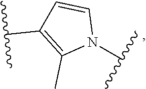 [SMILES: *c1ccn(*)c1C]

optionally substituted

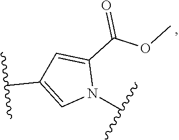 [SMILES: *c1cc(C(=O)OC)n(*)c1]

optionally substituted

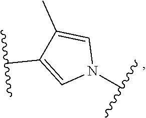 [SMILES: *c1cn(*)cc1C]

optionally substituted

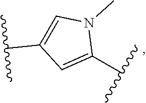 [SMILES: *c1cc(*)n(C)c1]

optionally substituted

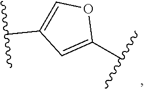 [SMILES: *c1coc(*)c1]

optionally substituted

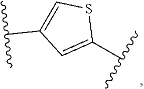 [SMILES: *c1csc(*)c1]

optionally substituted

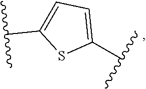 [SMILES: *c1ccc(*)s1]

optionally substituted

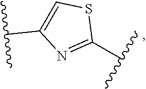 [SMILES: *c1csc(*)n1]

and optionally substituted

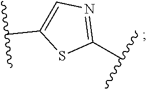 [SMILES: *c1cnc(*)s1]

W may be absent or W may be selected from the group consisting of the following structures that are optionally substituted:

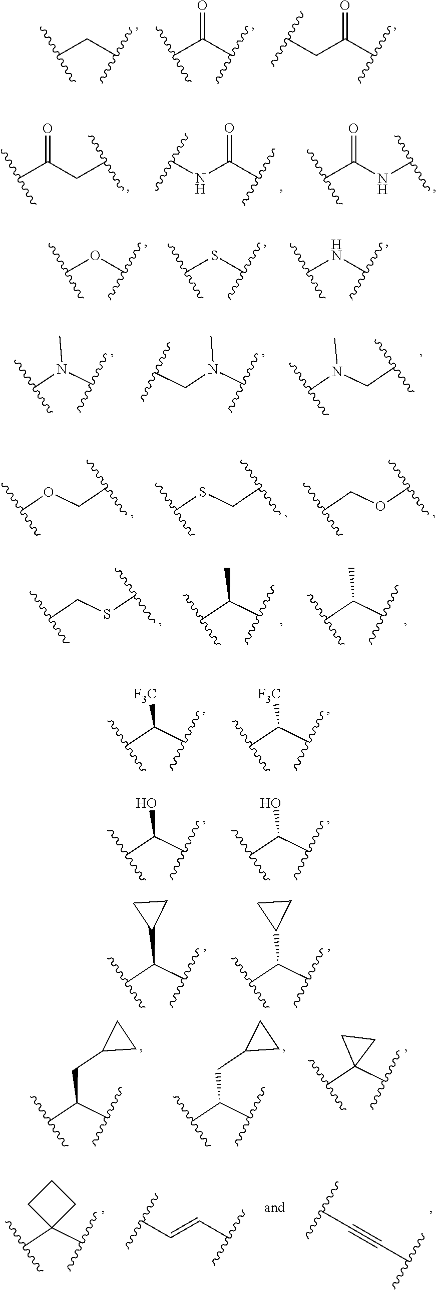

wherein when B may be optionally substituted

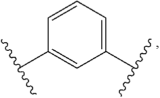 [SMILES: *c1cccc(*)c1]

### W may not be —S—;

Y may be H, and yn may be a number greater than 0;
Lx may comprise -L³-L²-L^{1x},
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

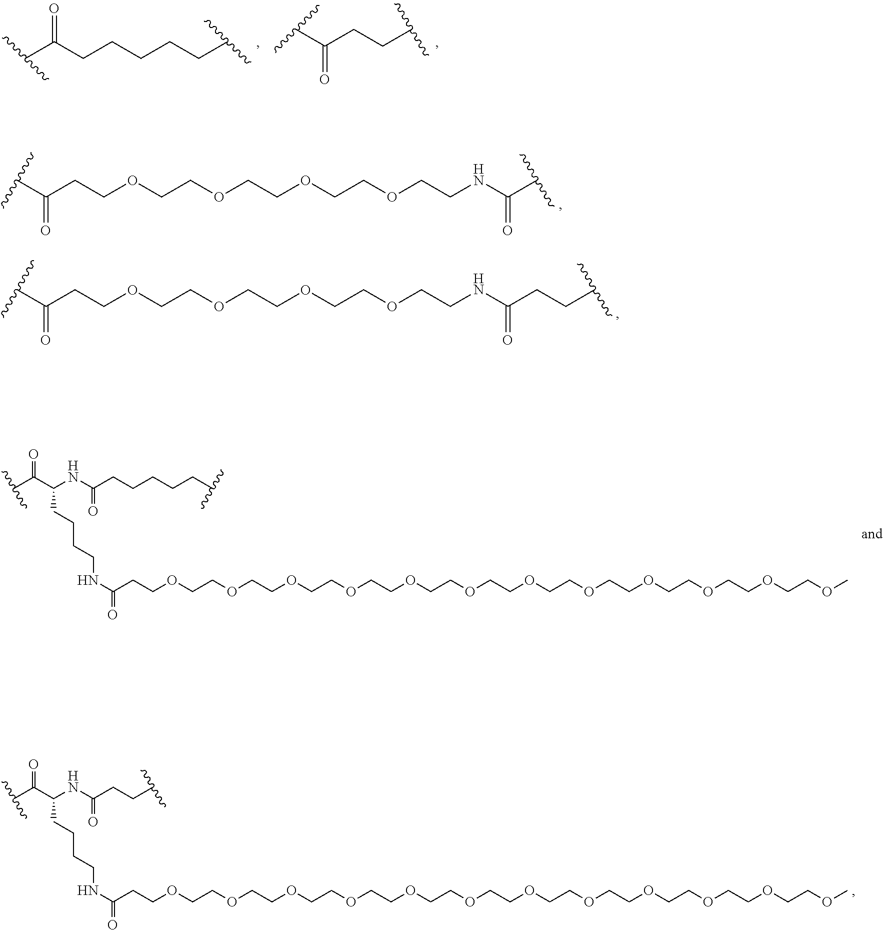 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L^{1x} may be selected from the group consisting of:

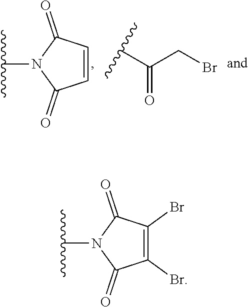

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-D), formula (II-b2-D), formula (II-b3-D) or formula (II-b4-D):

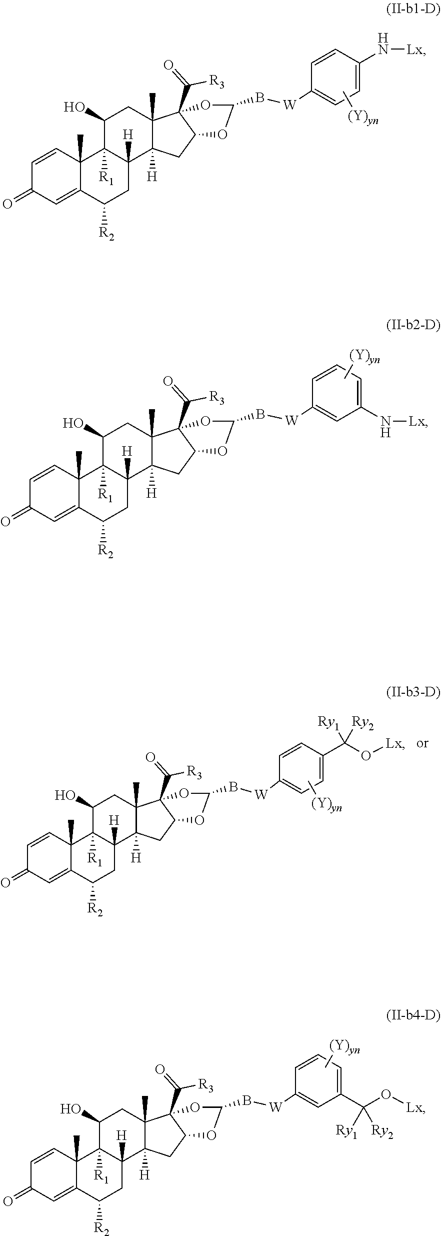

wherein
Ry₁ may be selected from the group consisting of hydrogen and optionally substituted methyl,
Ry₂ may be selected from the group consisting of hydrogen and optionally substituted methyl,
R₁ may be selected from the group consisting of hydrogen and halogen, R₂ may be selected from the group consisting of: hydrogen and halogen, R₃ may be selected from the group consisting of —CH₂OH, —SCH₂-halogen,

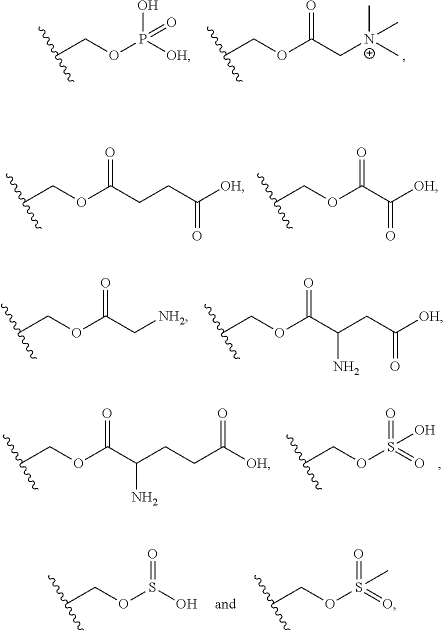

B may be selected from the group consisting of optionally substituted

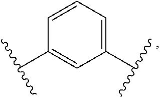 [SMILES: *c1cccc(*)c1]

optionally substituted

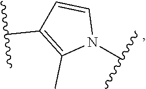 [SMILES: *c1ccn(*)c1C]

optionally substituted

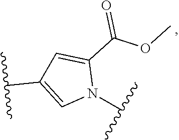 [SMILES: *c1cc(C(=O)OC)n(*)c1]

optionally substituted

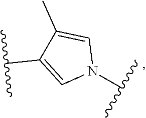 [SMILES: *c1cn(*)cc1C]

optionally substituted

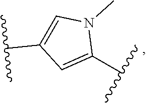 [SMILES: *c1cc(*)n(C)c1]

optionally substituted

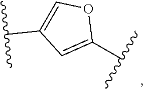 [SMILES: *c1coc(*)c1]

optionally substituted

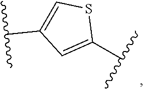 [SMILES: *c1csc(*)c1]

optionally substituted

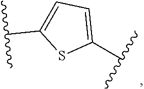 [SMILES: *c1ccc(*)s1]

optionally substituted

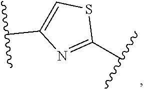 [SMILES: *c1csc(*)n1]

and optionally substituted

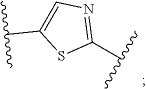 [SMILES: *c1cnc(*)s1]

W may be absent or W may be selected from the group consisting of the following structures that are optionally substituted: optionally substituted —CH₂—, optionally substituted —CH(CH₃)—, and optionally substituted

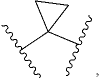 [SMILES: *C1(*)CC1]

Y may be H, and yn may be a number greater than 0;
Lx may comprise -L³-L²-L^{1x},
wherein L³ may be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² may be absent or selected from the group consisting of:

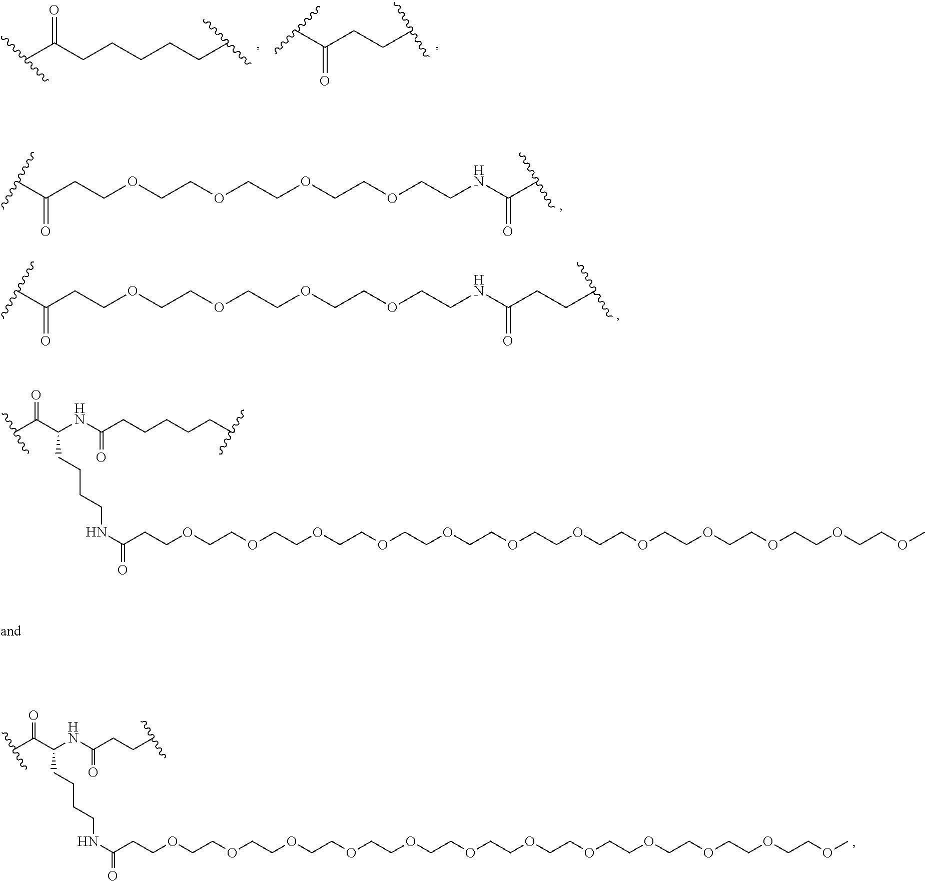 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

and
L^{1x} may be selected from the group consisting of:

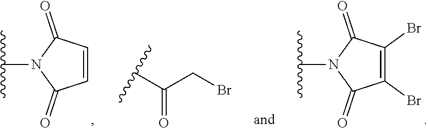 [SMILES: *N1C(=O)C=CC1=O ; *C(=O)CBr ; *N1C(=O)C(Br)=C(Br)C1=O]

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-E), formula (I-a2-E), formula (I-a3-E) or formula (I-a4-E):

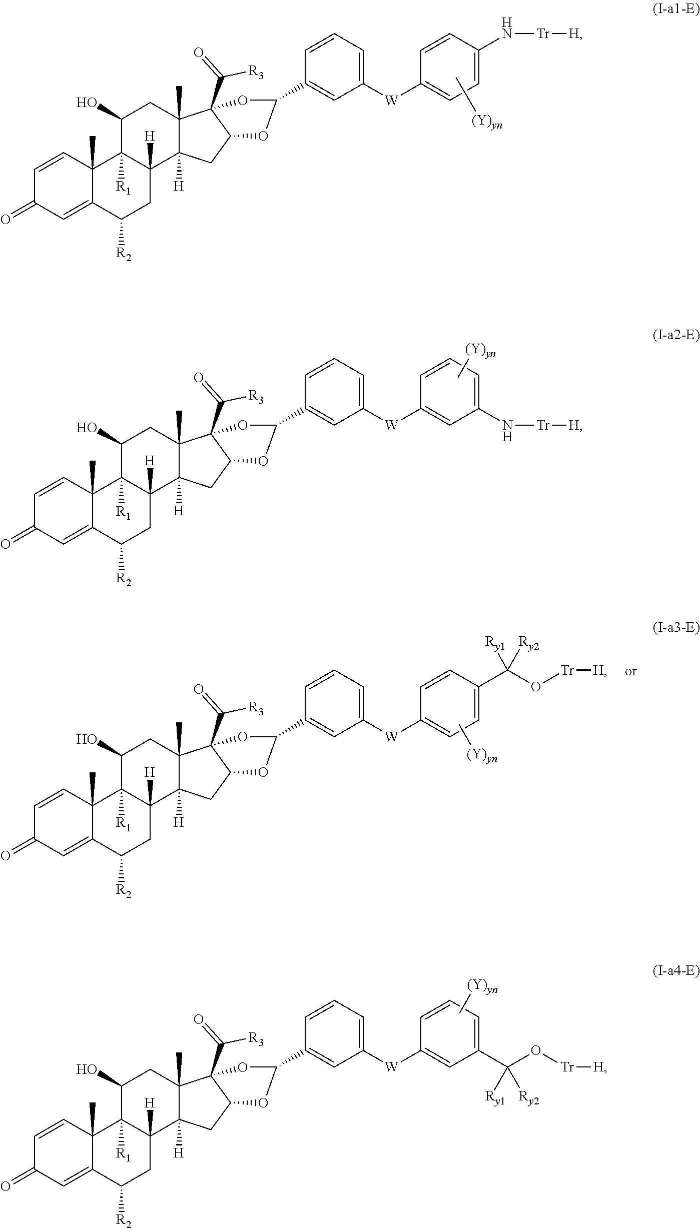

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

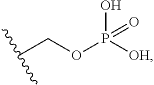 [SMILES: *COP(=O)(O)O]

optionally substituted

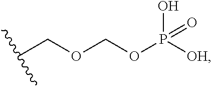 [SMILES: *COCOP(=O)(O)O]

optionally substituted

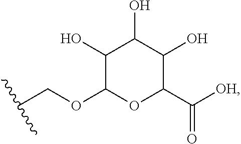 [SMILES: *COC1OC(C(=O)O)C(O)C(O)C1O]

optionally substituted

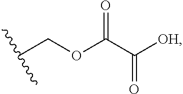 [SMILES: *COC(=O)C(=O)O]

optionally substituted

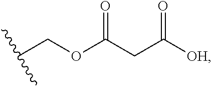 [SMILES: *COC(=O)CC(=O)O]

optionally substituted

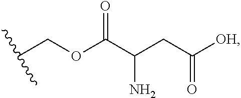 [SMILES: *COC(=O)C(N)CC(=O)O]

optionally substituted

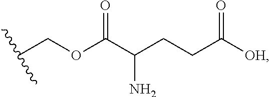 [SMILES: *COC(=O)C(N)CCC(=O)O]

optionally substituted

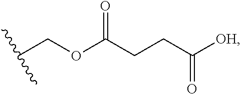 [SMILES: *COC(=O)CCC(=O)O]

optionally substituted

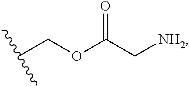 [SMILES: *COC(=O)CN]

optionally substituted

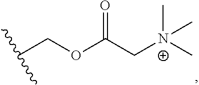 [SMILES: *COC(=O)C[N+](C)(C)C]

optionally substituted

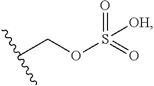 [SMILES: *COS(=O)(=O)O]

optionally substituted

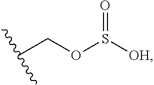 [SMILES: *COS(=O)O]

optionally substituted

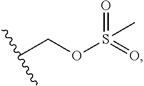 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Tr may be absent or may be selected from the group consisting of:

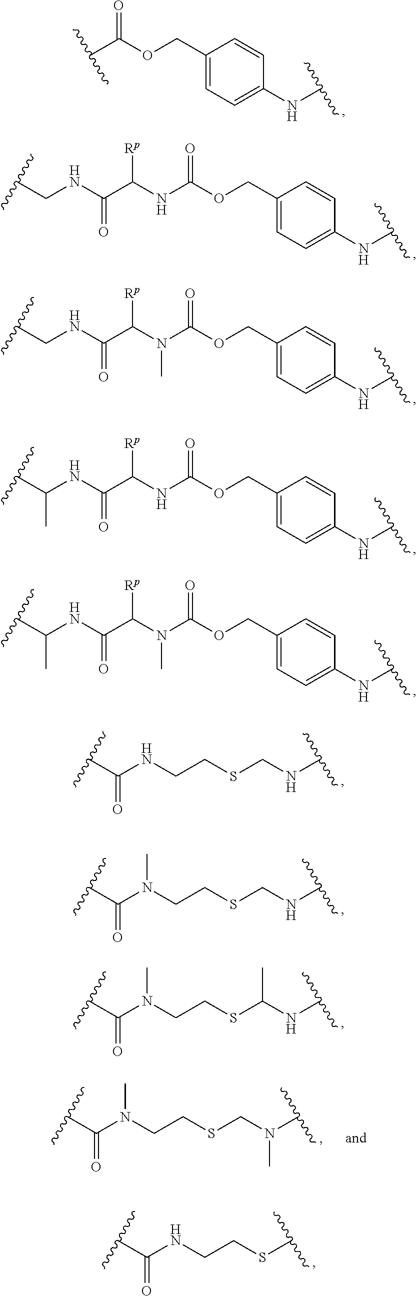

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (I-a1-E), formula (I-a2-E), formula (I-a3-E) or formula (I-a4-E):

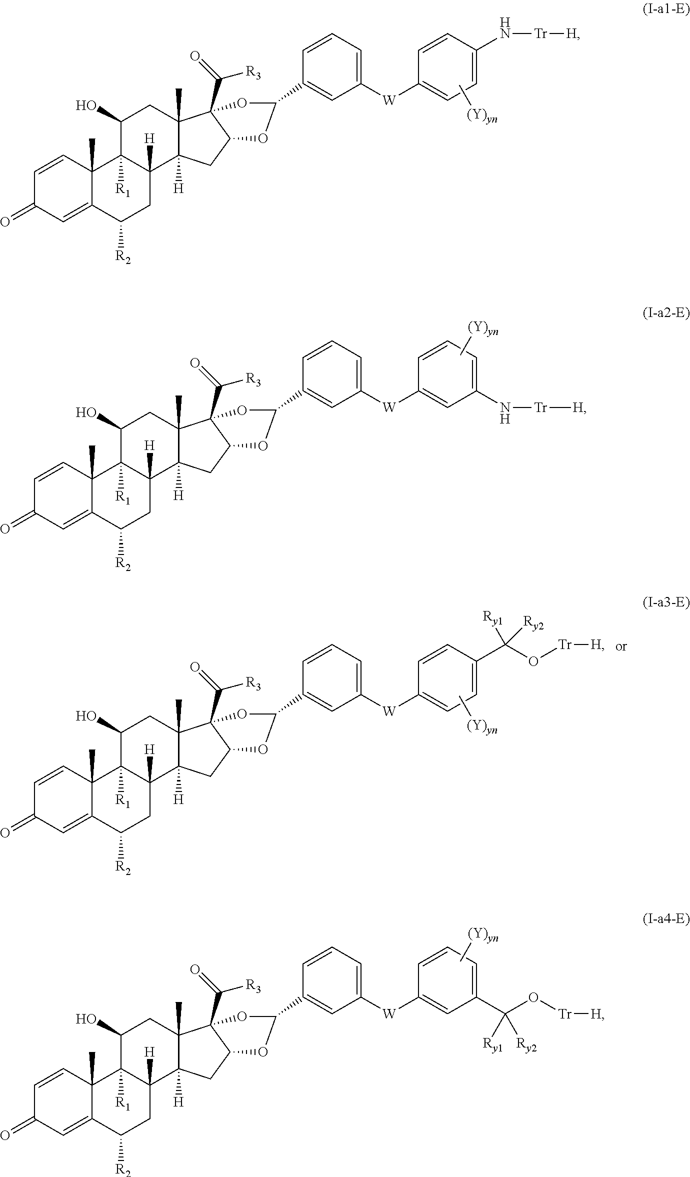

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂F,

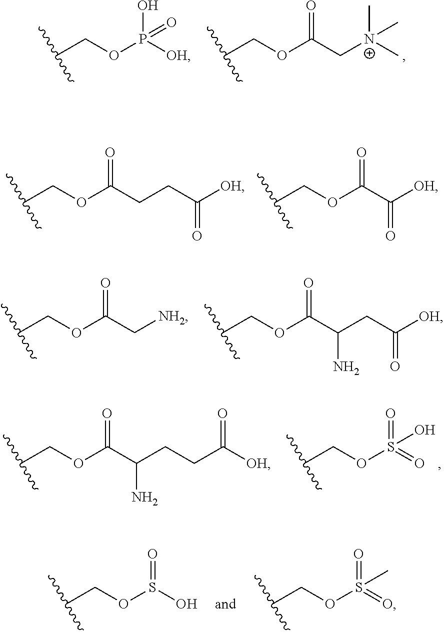

W may be absent or W may be optionally substituted —NCH₃—, optionally substituted —CH₂CH₂—, optionally substituted —CH═CH—, —C≡C—, optionally substituted —CO—NH—, optionally substituted —OCH₂—, optionally substituted —CH₂O—, optionally substituted —SCH₂—, optionally substituted —CH₂S—, or optionally substituted —NH—CO—,
Y may be H, and yn may be a number greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-E), formula (II-a2-E), formula (II-a3-E) or formula (II-a4-E):

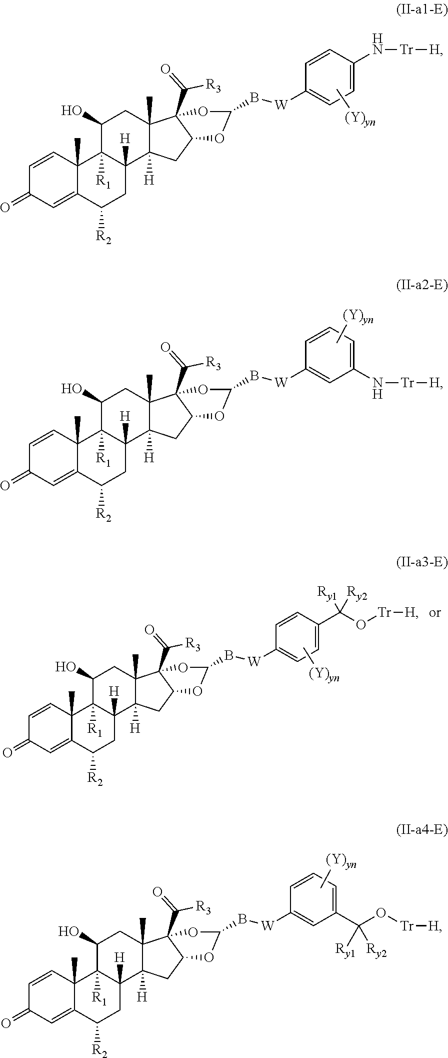

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

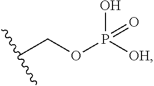 [SMILES: *COP(=O)(O)O]

optionally substituted

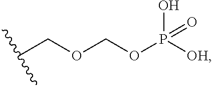 [SMILES: *COCOP(=O)(O)O]

optionally substituted

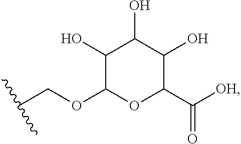 [SMILES: *COC1OC(C(=O)O)C(O)C(O)C1O]

optionally substituted

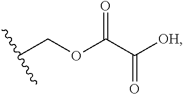 [SMILES: *COC(=O)C(=O)O]

optionally substituted

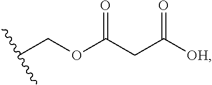 [SMILES: *COC(=O)CC(=O)O]

optionally substituted

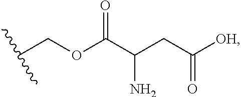 [SMILES: *COC(=O)C(N)CC(=O)O]

optionally substituted

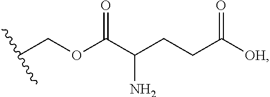 [SMILES: *COC(=O)C(N)CCC(=O)O]

optionally substituted

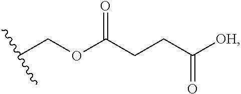 [SMILES: *COC(=O)CCC(=O)O]

optionally substituted

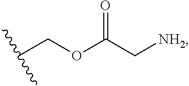 [SMILES: *COC(=O)CN]

optionally substituted

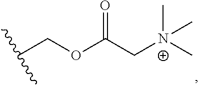 [SMILES: *COC(=O)C[N+](C)(C)C]

optionally substituted

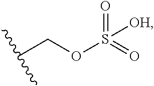 [SMILES: *COS(=O)(=O)O]

optionally substituted

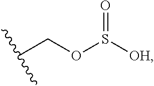 [SMILES: *COS(=O)O]

optionally substituted

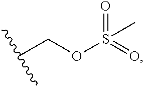 [SMILES: *COS(C)(=O)=O]

optionally substituted —OH, optionally substituted —OCH₃, optionally substituted —OCH₂F, optionally substituted —OCH₂C, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

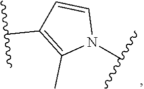 [SMILES: *c1ccn(*)c1C]

optionally substituted

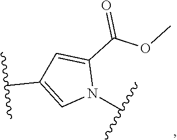 [SMILES: *c1cc(C(=O)OC)n(*)c1]

optionally substituted

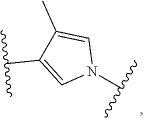 [SMILES: *c1cn(*)cc1C]

optionally substituted

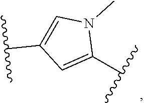 [SMILES: *c1cc(*)n(C)c1]

optionally substituted

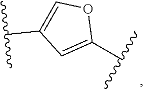 [SMILES: *c1coc(*)c1]

optionally substituted

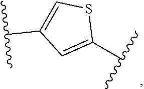 [SMILES: *c1csc(*)c1]

optionally substituted

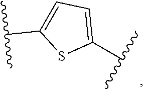 [SMILES: *c1ccc(*)s1]

optionally substituted

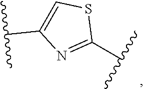 [SMILES: *c1csc(*)n1]

and optionally substituted

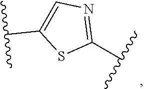 [SMILES: *c1cnc(*)s1]

W may be absent or W may be selected from the group consisting of the following structures that are optionally substituted:

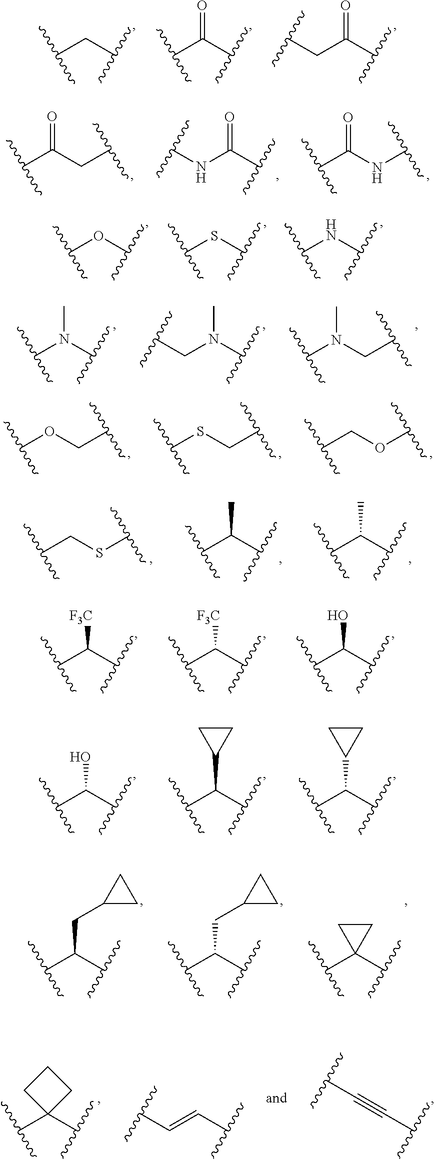

Y may be H and yn may be a number greater than 0;
Tr may be absent or may be selected from the group consisting of:

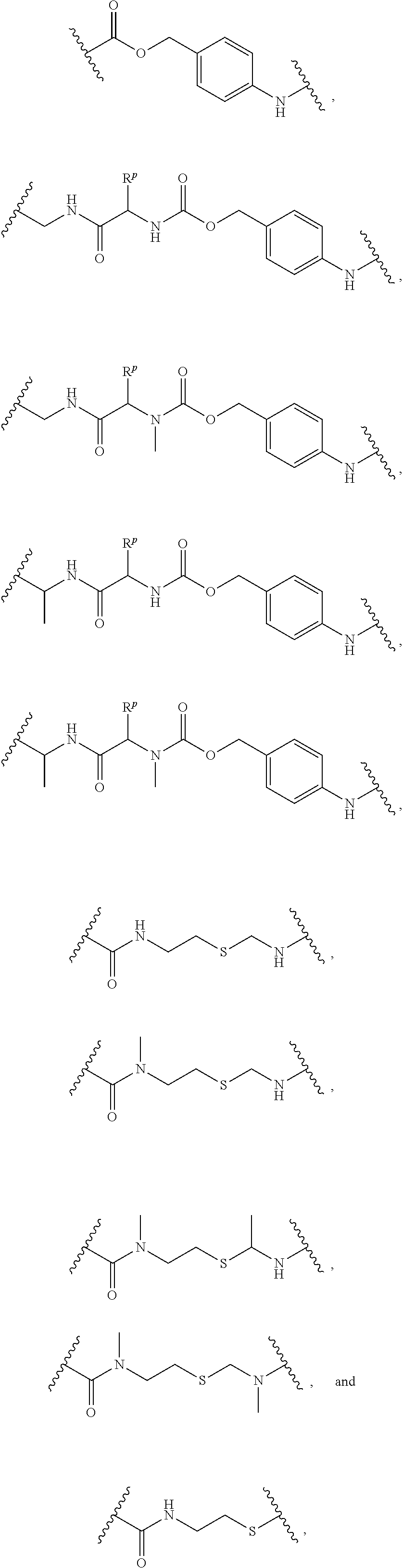

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH2, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-E), formula (II-a2-E), formula (II-a3-E) or formula (II-a4-E):

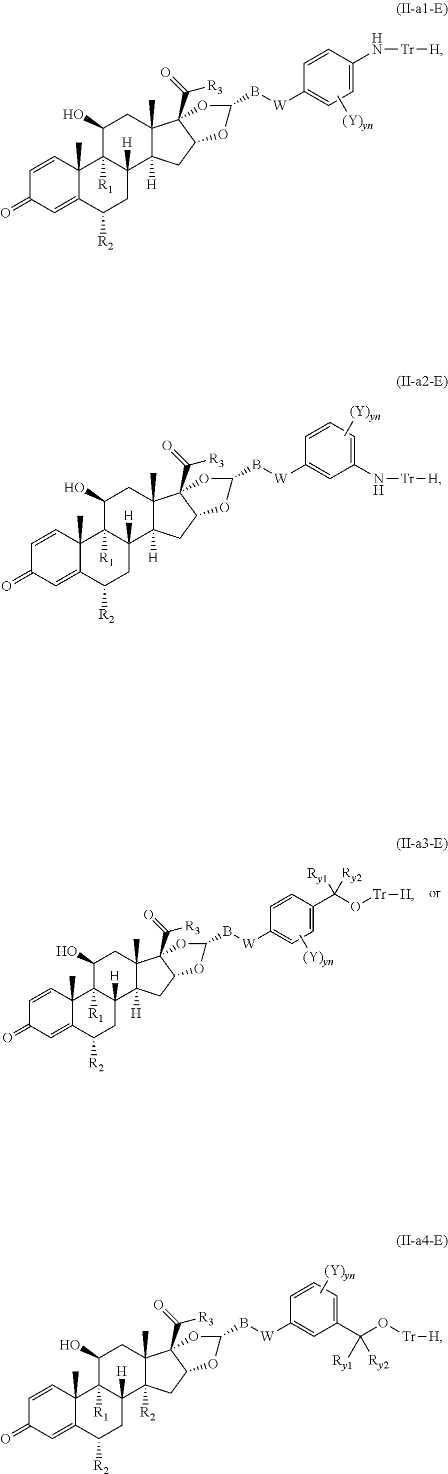

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of —CH₂OH, —SCH₂-halogen,

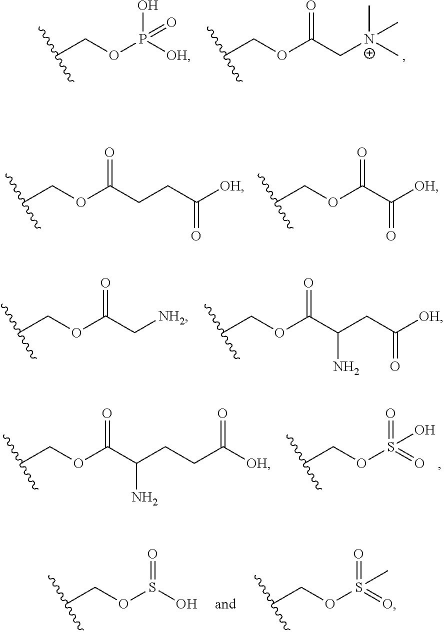

B may be selected from the group consisting of: optionally substituted

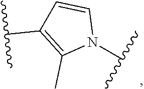 [SMILES: *c1ccn(*)c1C]

optionally substituted

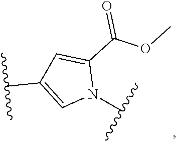 [SMILES: *c1cc(C(=O)OC)n(*)c1]

optionally substituted

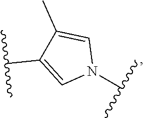 [SMILES: *c1cn(*)cc1C]

optionally substituted

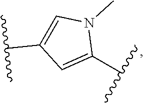 [SMILES: *c1cc(*)n(C)c1]

optionally substituted

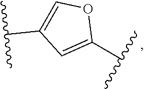 [SMILES: *c1coc(*)c1]

optionally substituted

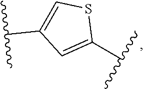 [SMILES: *c1csc(*)c1]

optionally substituted

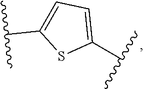 [SMILES: *c1ccc(*)s1]

optionally substituted

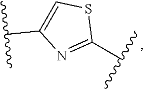 [SMILES: *c1csc(*)n1]

and optionally substituted

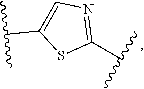 [SMILES: *c1cnc(*)s1]

W may be absent or W may be selected from the group consisting of the following structures that are optionally substituted: optionally substituted —CH₂—, optionally substituted —CH(CH₃)—, and optionally substituted

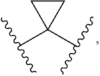 [SMILES: *C1(*)CC1]

Y may be H, and yn may be a n-umber greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-E), formula (II-b2-E), formula (II-b3-E) or formula (II-b4-E):

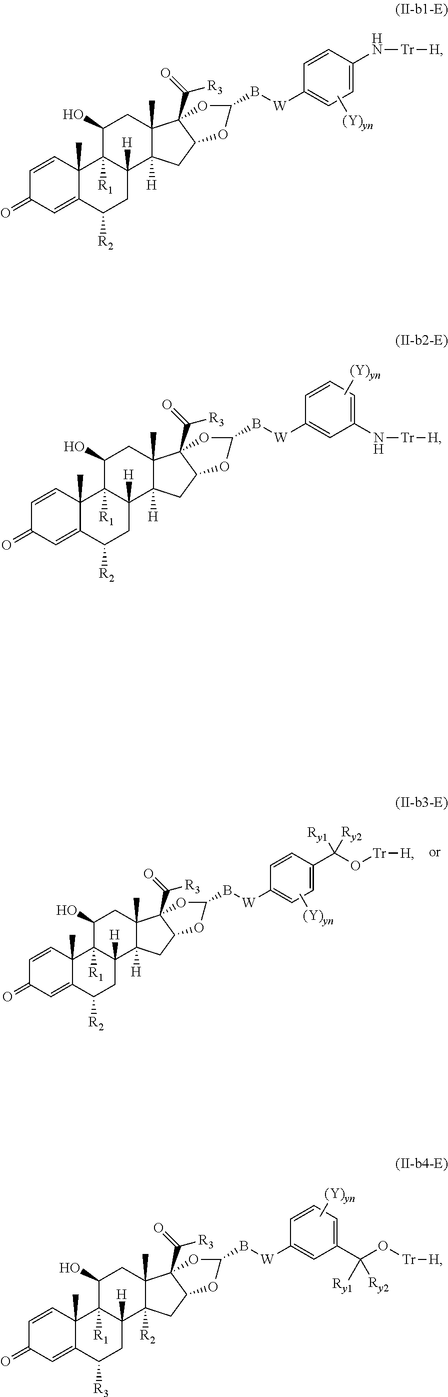

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen, halogen, and optionally substituted methyl, R₂ may be selected from the group consisting of: hydrogen, halogen, and optionally substituted methyl, R₃ may be selected from the group consisting of: optionally substituted —CH₂Cl, optionally substituted —CH₂SH, optionally substituted —CH₂OH, optionally substituted

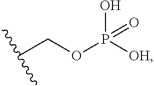 [SMILES: *COP(=O)(O)O]

optionally substituted

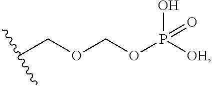 [SMILES: *COCOP(=O)(O)O]

optionally substituted

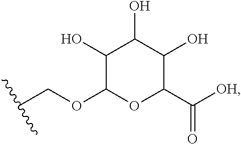 [SMILES: *COC1OC(C(=O)O)C(O)C(O)C1O]

optionally substituted

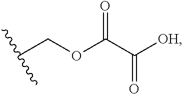 [SMILES: *COC(=O)C(=O)O]

optionally substituted

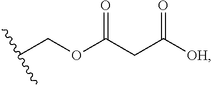 [SMILES: *COC(=O)CC(=O)O]

optionally substituted

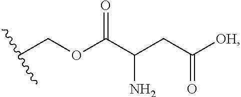 [SMILES: *COC(=O)C(N)CC(=O)O]

optionally substituted

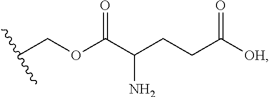 [SMILES: *COC(=O)C(N)CCC(=O)O]

optionally substituted

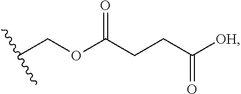 [SMILES: *COC(=O)CCC(=O)O]

optionally substituted

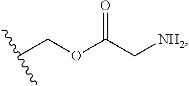 [SMILES: *COC(=O)CN]

optionally substituted

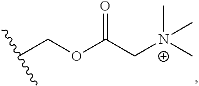 [SMILES: *COC(=O)C[N+](C)(C)C]

optionally substituted

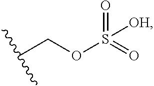 [SMILES: *COS(=O)(=O)O]

optionally substituted

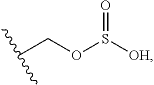 [SMILES: *COS(=O)O]

optionally substituted

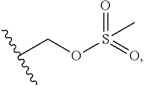 [SMILES: *COS(C)(=O)=O]

optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃, and optionally substituted —SCH₂CN,
B may be selected from the group consisting of: optionally substituted

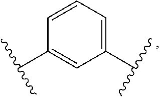 [SMILES: *c1cccc(*)c1]

optionally substituted

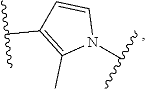 [SMILES: *c1ccn(*)c1C]

optionally substituted

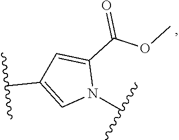 [SMILES: *c1cc(C(=O)OC)n(*)c1]

optionally substituted

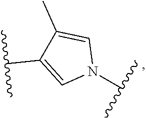 [SMILES: *c1cn(*)cc1C]

optionally substituted

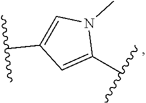 [SMILES: *c1cc(*)n(C)c1]

optionally substituted

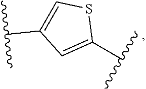 [SMILES: *c1csc(*)c1]

optionally substituted

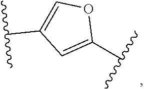 [SMILES: *c1coc(*)c1]

optionally substituted

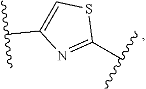 [SMILES: *c1csc(*)n1]

optionally substituted

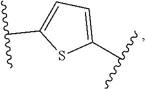 [SMILES: *c1ccc(*)s1]

and optionally substituted

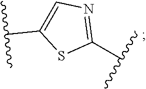 [SMILES: *c1cnc(*)s1]

W may be absent or W may be selected from the group consisting of the following structures that are optionally substituted:

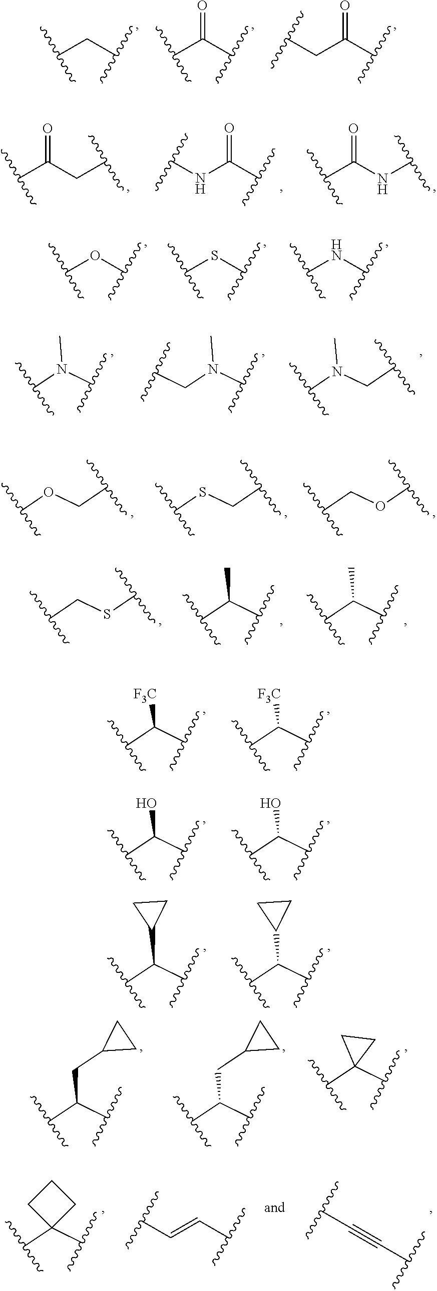

wherein when B may be optionally substituted

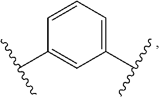 [SMILES: *c1cccc(*)c1]

### W may not be —S—;

Y may be H, and yn may be a number greater than 0;
Tr may be absent or may be selected from the group consisting of:

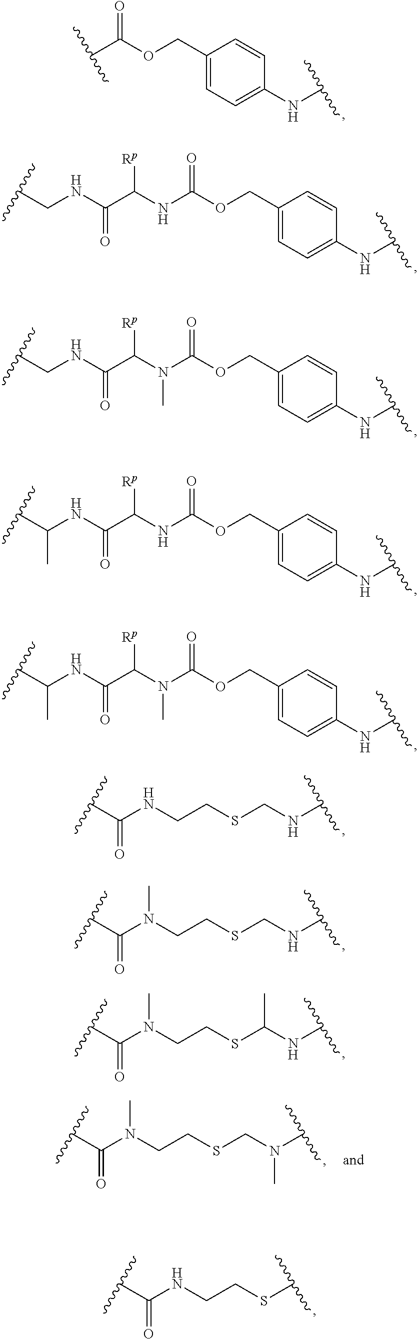

wherein R^{p} may be selected from the group consisting of: H, —CH₃, —CH—(CH₃)₂, —CH₂—CH(CH₃)₂, —CH(CH₃)—CH₂—CH₃, —CH₂—C₆H₅, —C₈NH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH)NH₂ and —(CH₂)₃—NHCONH₂.

The present application provides a compound or a tautomer, a mesomer, a racemate, an enantiomer or a diastereoisomer thereof, or a mixture thereof, or a pharmaceutically acceptable salt thereof, wherein the compound comprises a structure shown as formula (II-b1-E), formula (II-b2-E), formula (II-b3-E) or formula (II-b4-E):

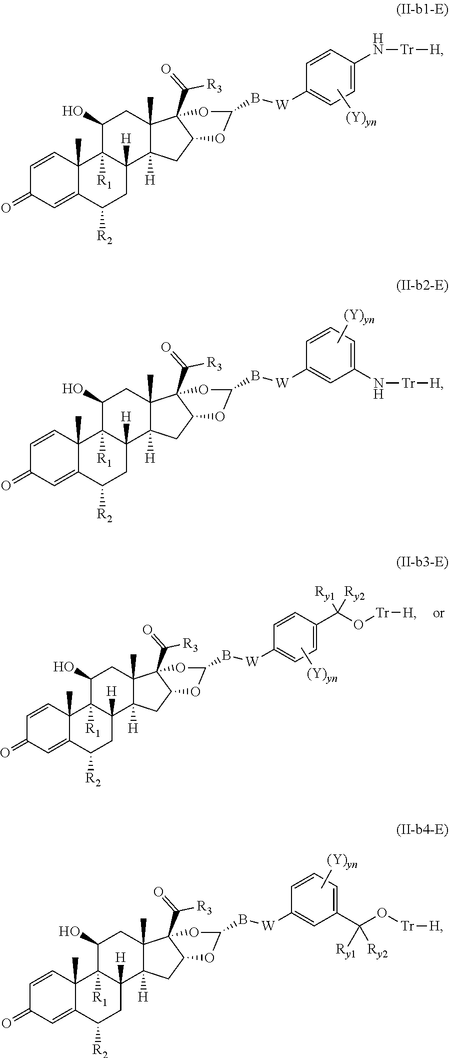

wherein
Ry₁ may be selected from the group consisting of: hydrogen and optionally substituted methyl,
Ry₂ may be selected from the group consisting of: hydrogen and optionally substituted methyl,
R₁ may be selected from the group consisting of: hydrogen and halogen, R₂ may be selected from the group consisting of: hydrogen and halogen, R₃ may be selected from the group consisting of: —CH₂OH, —SCH₂-halogen,

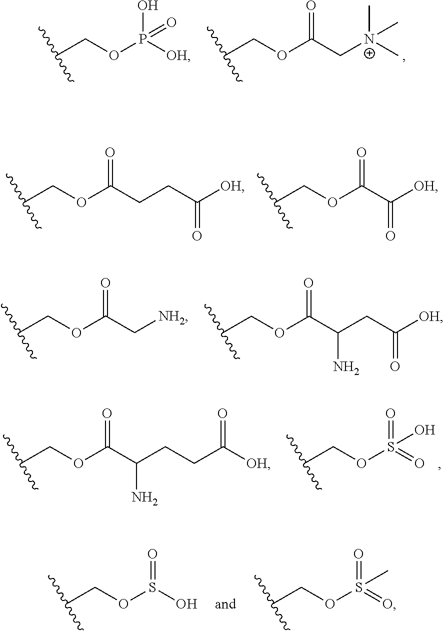

B may be selected from the group consisting of: optionally substituted

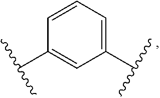 [SMILES: *c1cccc(*)c1]

optionally substituted

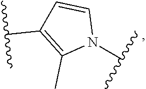 [SMILES: *c1ccn(*)c1C]

optionally substituted

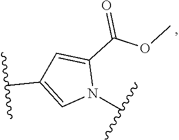 [SMILES: *c1cc(C(=O)OC)n(*)c1]

optionally substituted

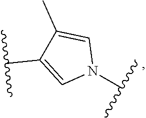 [SMILES: *c1cn(*)cc1C]

optionally substituted

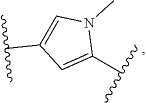 [SMILES: *c1cc(*)n(C)c1]

optionally substituted

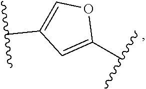 [SMILES: *c1coc(*)c1]

optionally substituted

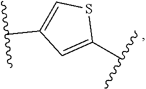 [SMILES: *c1csc(*)c1]

optionally substituted

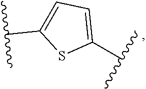 [SMILES: *c1ccc(*)s1]

optionally substituted

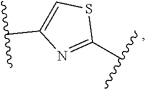 [SMILES: *c1csc(*)n1]

and optionally substituted

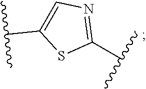 [SMILES: *c1cnc(*)s1]

W may be absent or W may be selected from the group consisting of the following structures that are optionally substituted: optionally substituted —CH₂—, optionally substituted —CH(CH₃)—, and optionally substituted

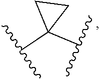 [SMILES: *C1(*)CC1]

Y may be H, and yn may be a number greater than 0;
Tr may be absent.

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group consisting of:

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group consisting of:

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group consisting of:

| No. | Structure |
|---|---|
| I-D-1 | 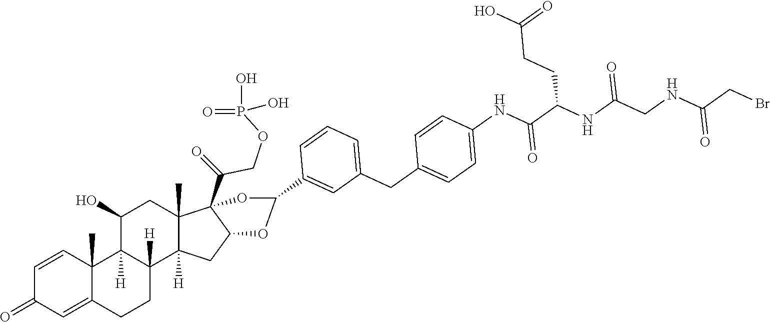 |
| I-D-2 | 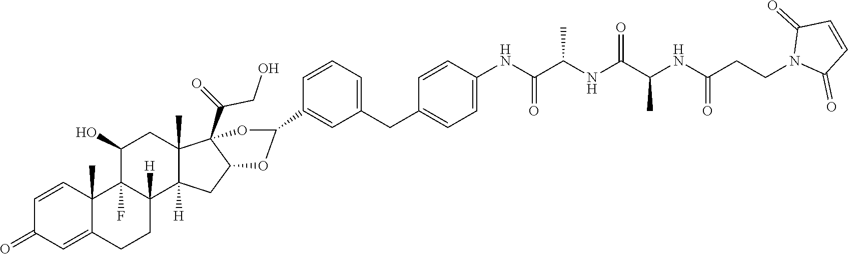 |
| I-D-3 | 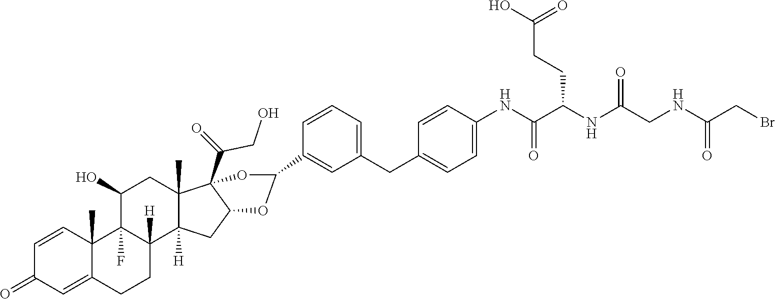 |
| I-D-4 | 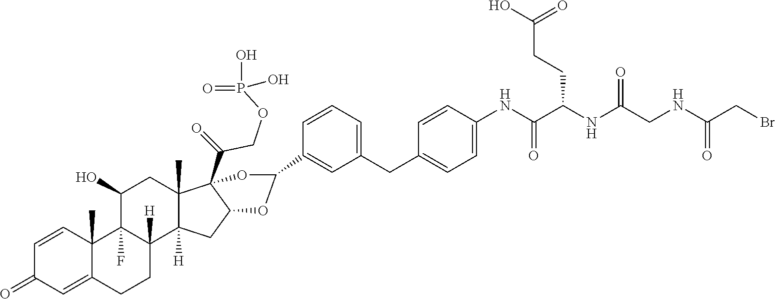 |
| I-D-5 | 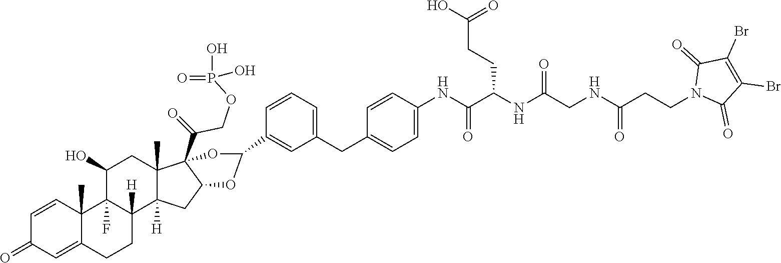 |
| I-D-10 | 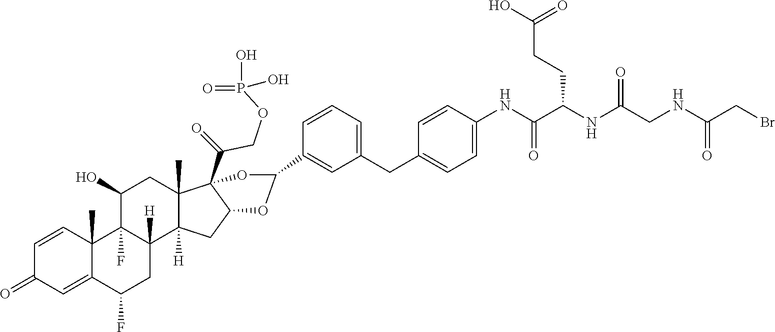 |
| I-D-15 | 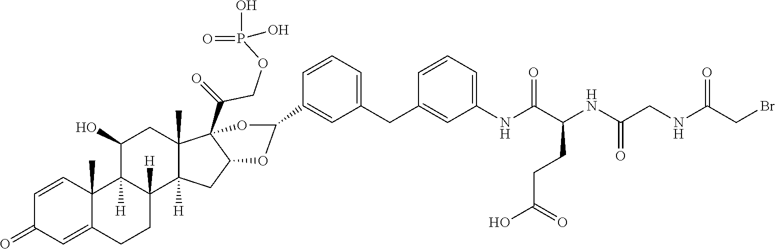 |
| I-D-16 | 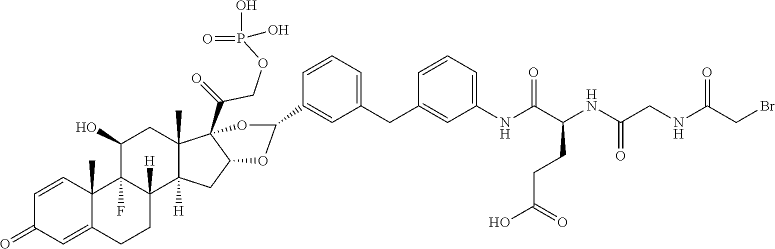 |
| I-D-17 | 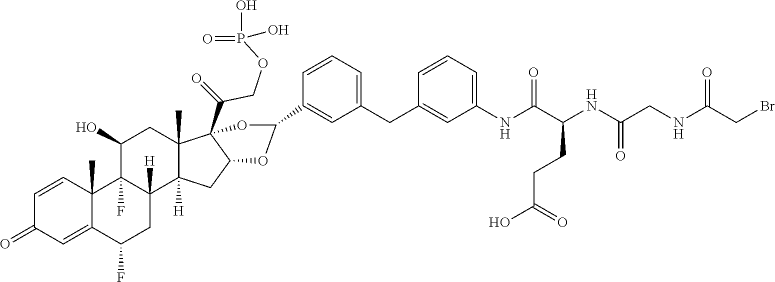 |
| I-D-18 | 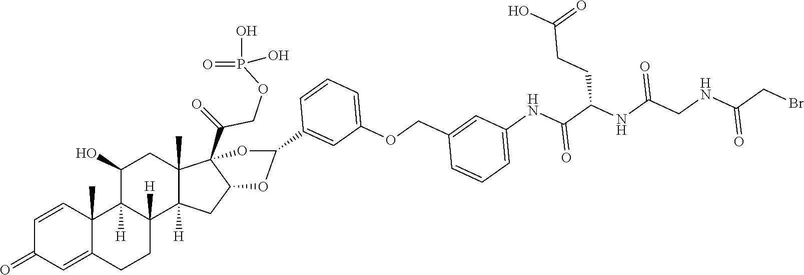 |
| I-D- 20 | 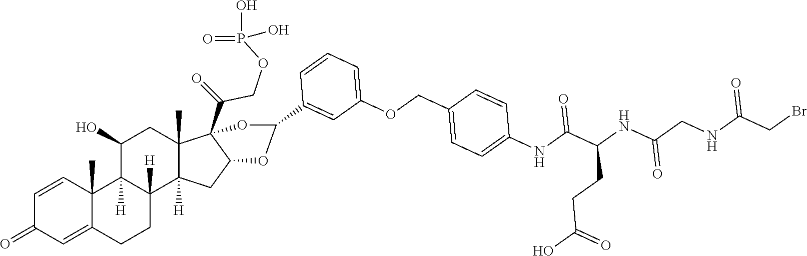 |
| I-D- 32 | 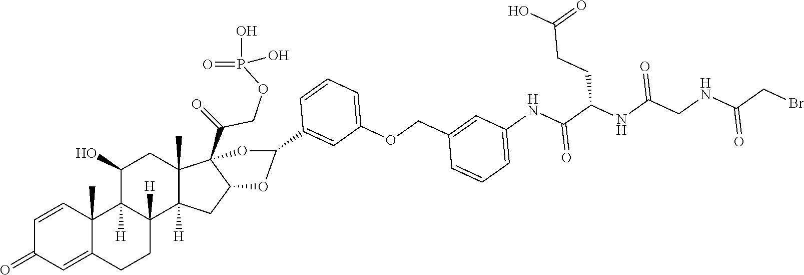 |
| I-D- 34 | 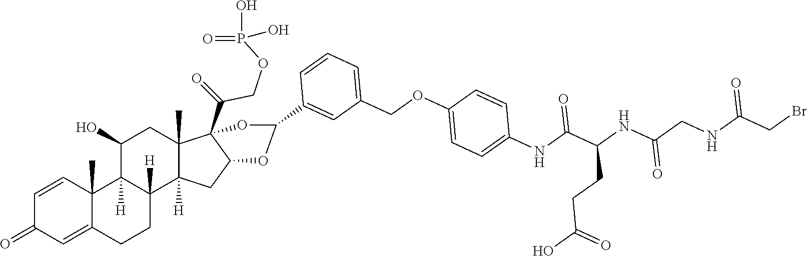 |
| I-D- 46 | 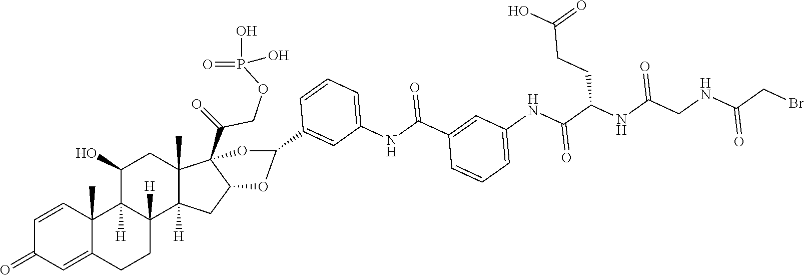 |
| I-D- 48 | 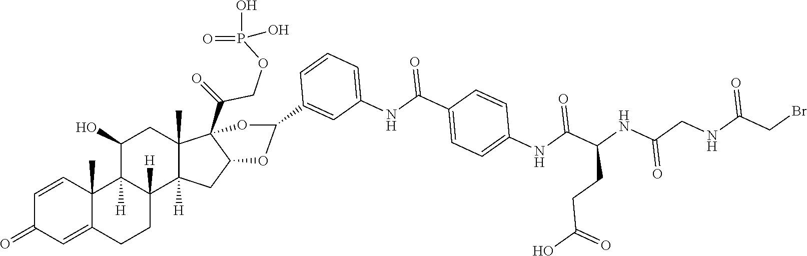 |
| I-D- 50 | 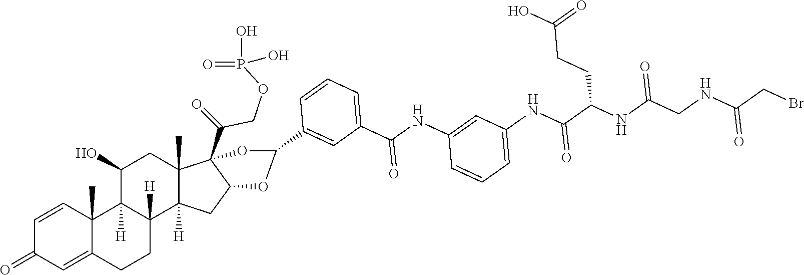 |
| I-D- 52 | 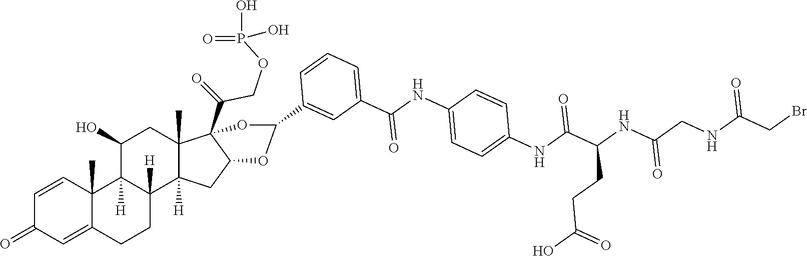 |
| I-D- 54 | 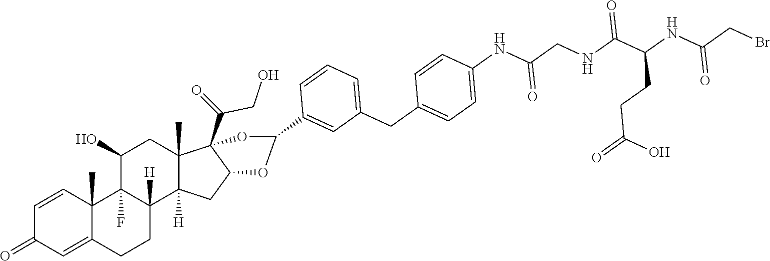 |
| I-D- 55 | 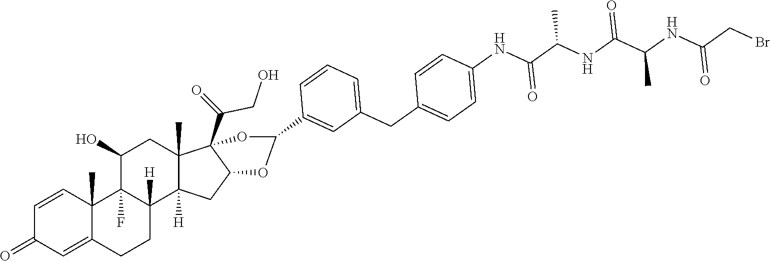 |
| I-D- 56 | 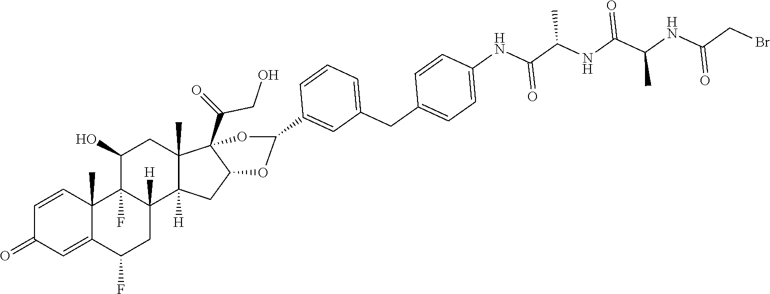 |
| I-D- 57 | 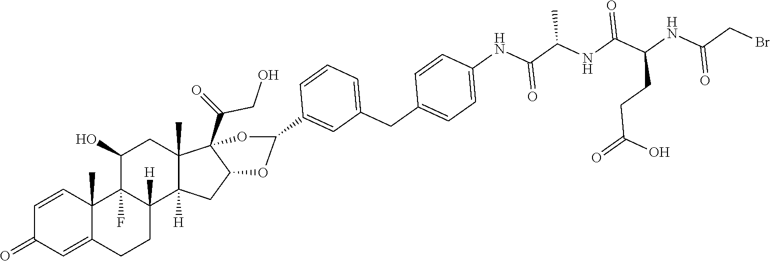 |
| I-D- 58 | 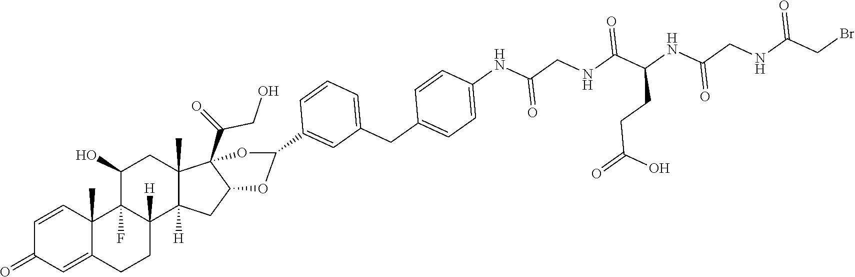 |
| I-D- 59 | 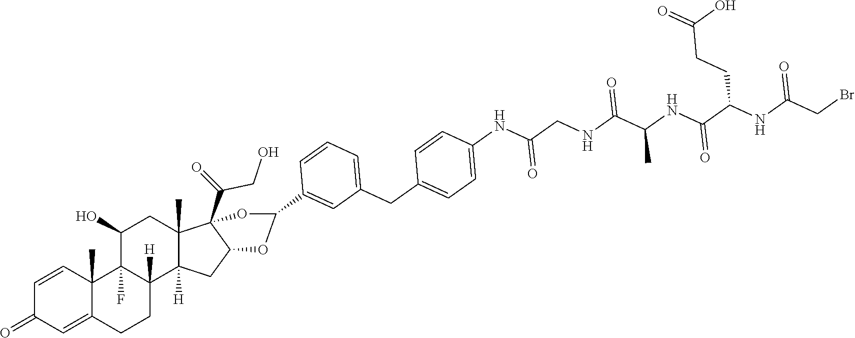 |
| I-D- 60 | 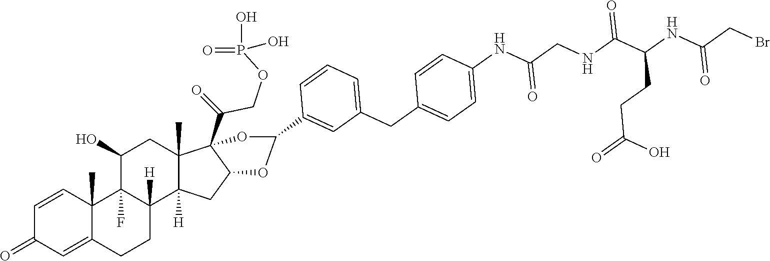 |
| I-D- 61 | 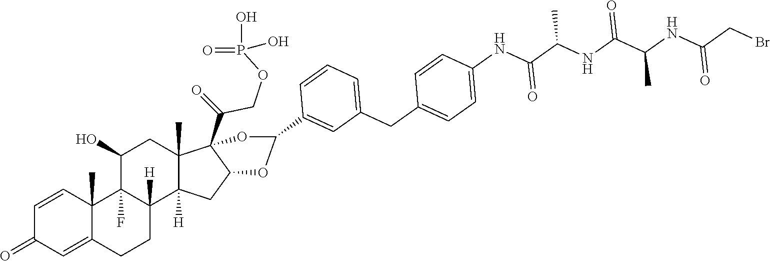 |
| I-D-62 | 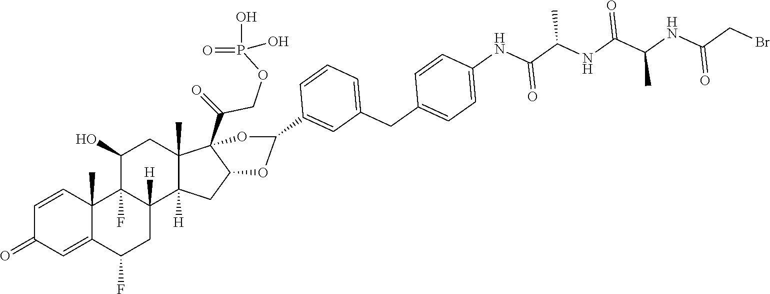 |
| I-D- 63 | 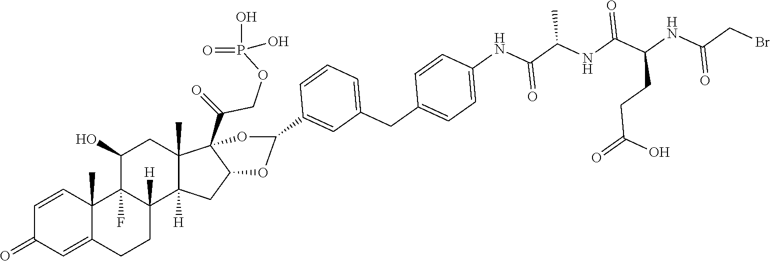 |
| I-D- 64 | 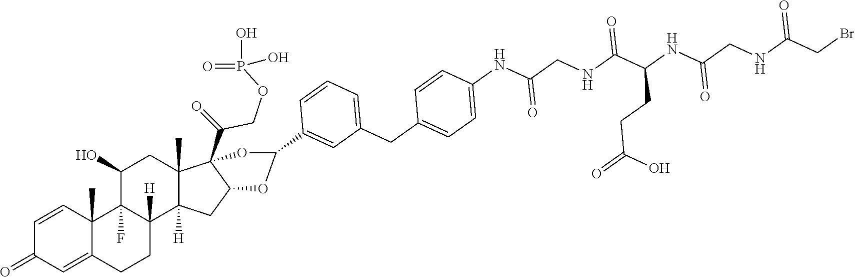 |
| I-D- 65 | 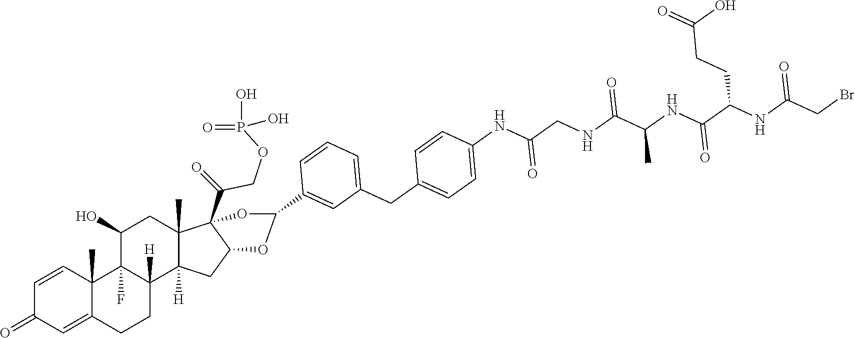 |
| I-D- 66 | 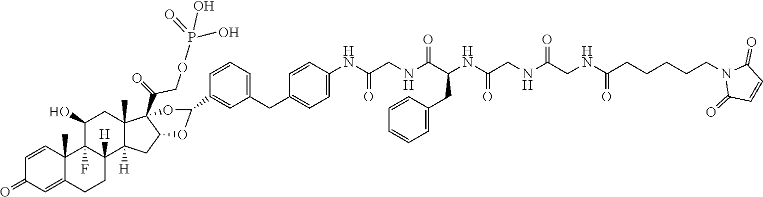 |
| I-D- 67 | 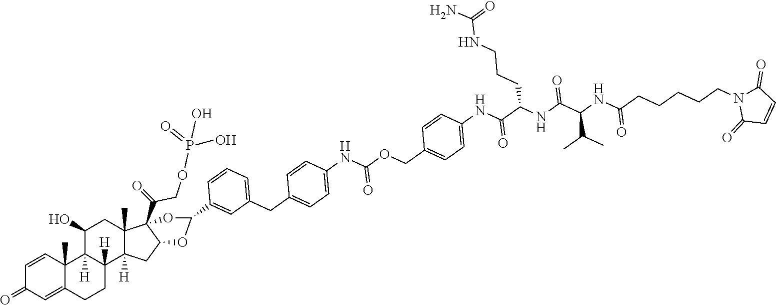 |
| I-D- 68 | 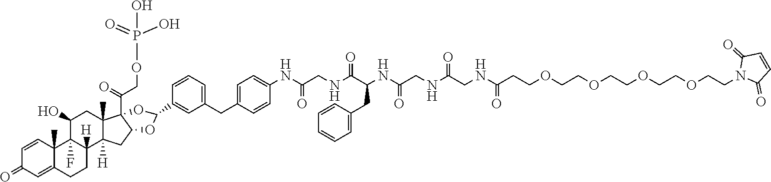 |
| I-D- 69 | 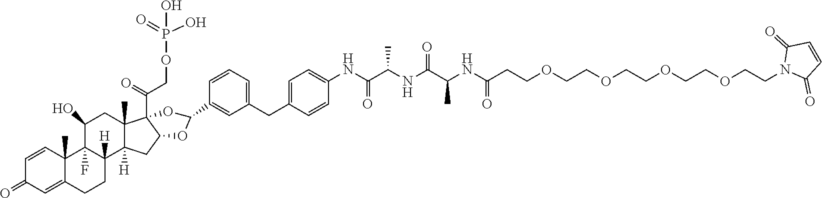 |
| I-D- 70 | 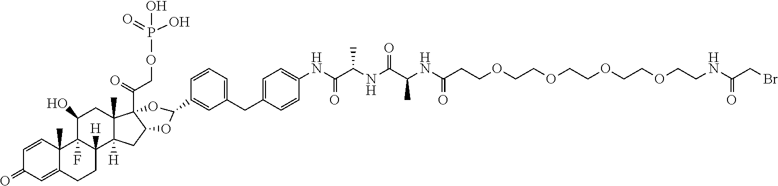 |
| I-D- 71 | 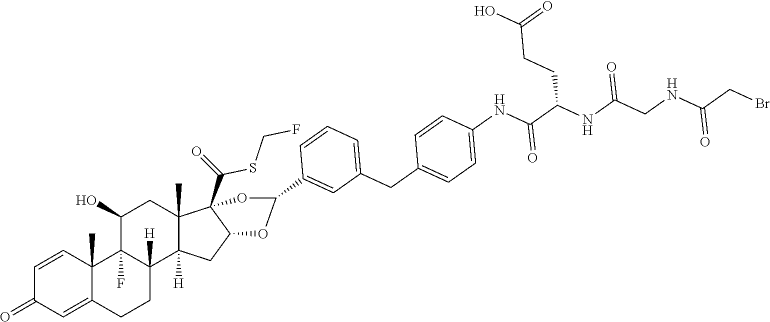 |
| I-D- 72 | 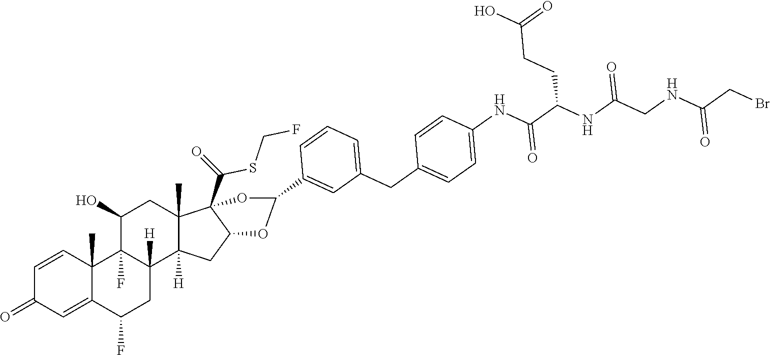 |
| I-D- 73 | 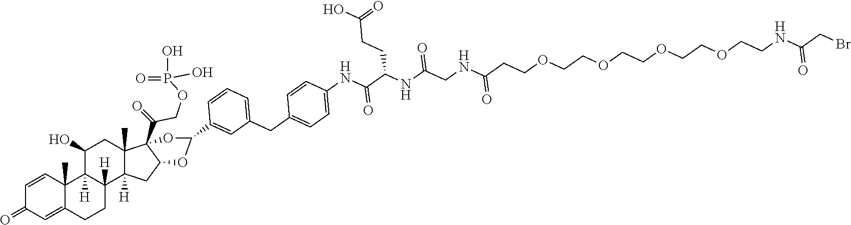 |
| I-D- 74 | 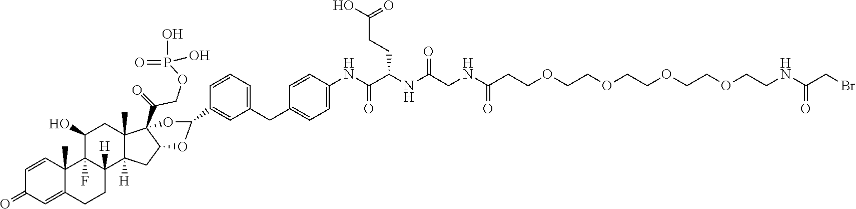 |
| I-D- 75 | 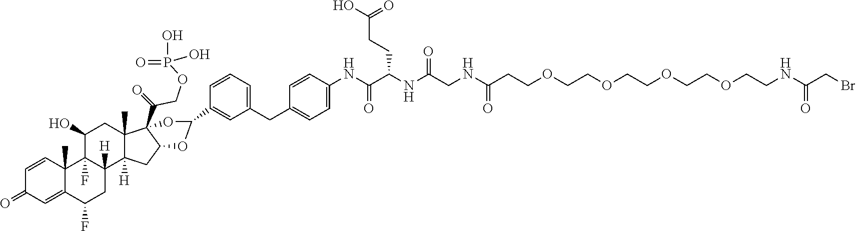 |
| I-D- 76 | 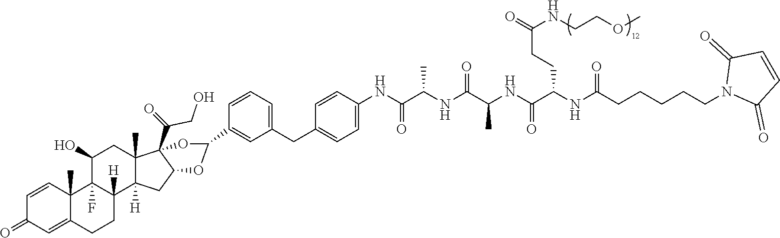 |
| I-D- 77 | 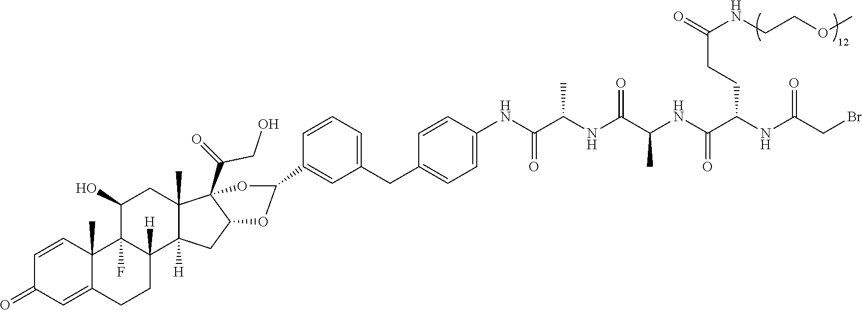 |

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group consisting of:

| No. | Structure |
|---|---|
| II-D-1 | 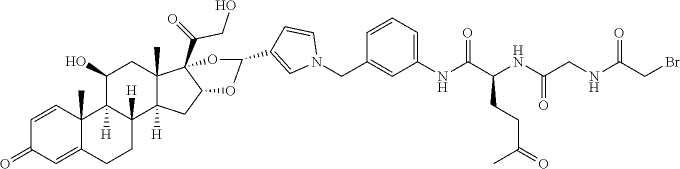 |
| II-D-2 | 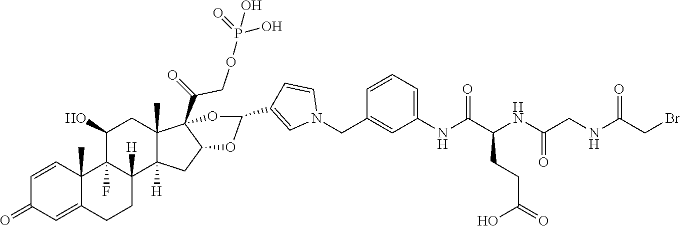 |
| II-D-7 | 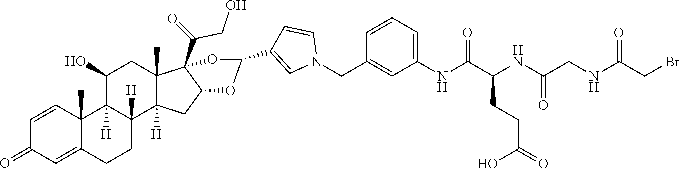 |
| II-D-8 | 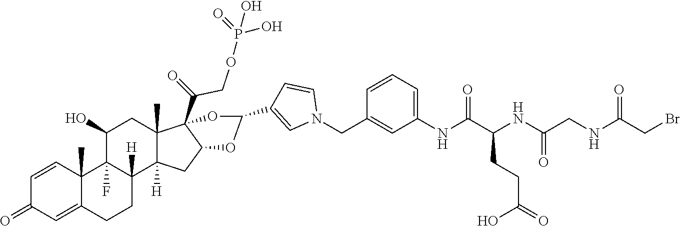 |
| II-D-12 | 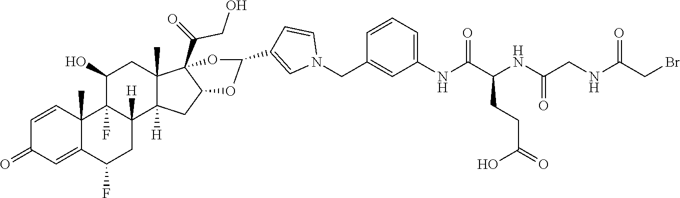 |
| II-D-13 | 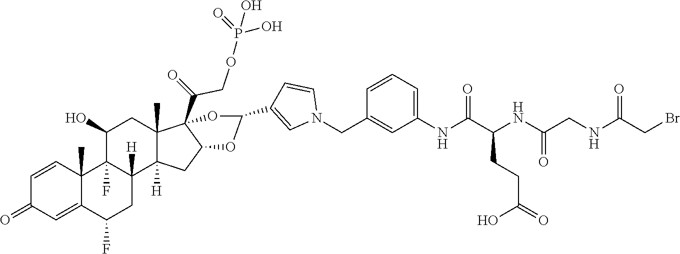 |
| II-D-17 | 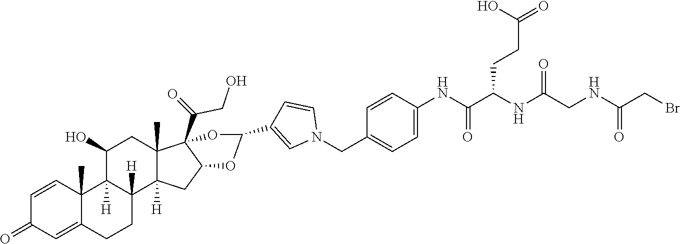 |
| II-D-18 | 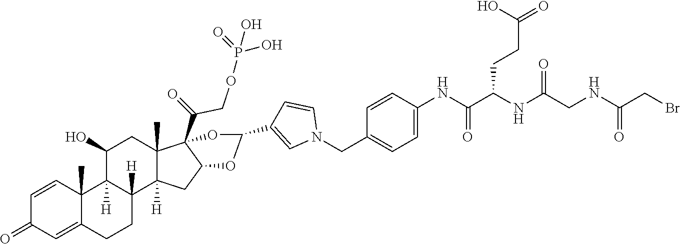 |
| II-D-23 | 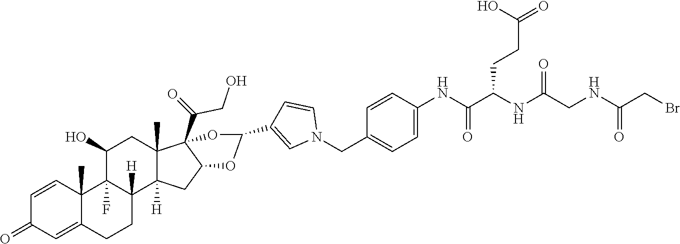 |
| II-D-24 | 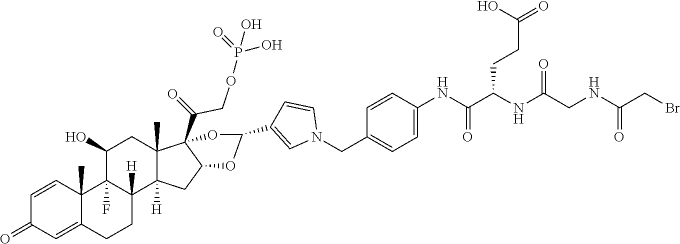 |
| II-D-29 | 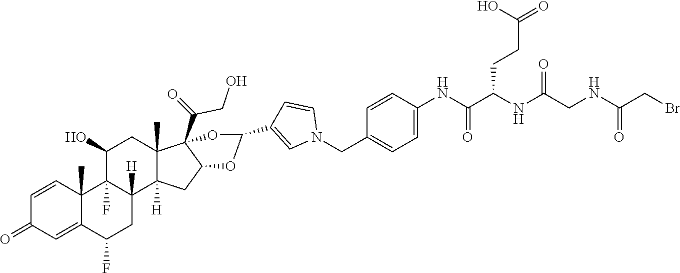 |
| II-D-30 | 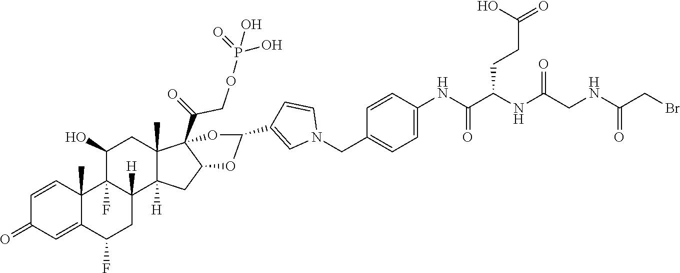 |
| II-D-36 | 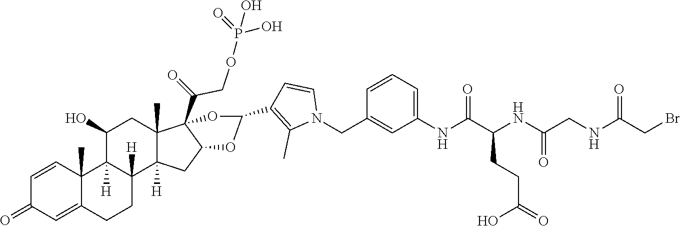 |
| II-D-39 | 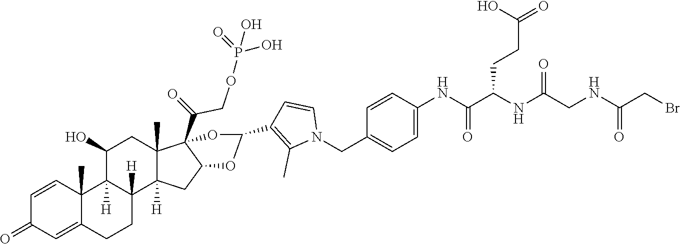 |
| II-D-42 | 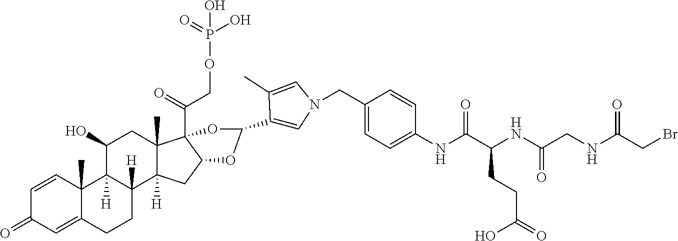 |
| II-D-45 | 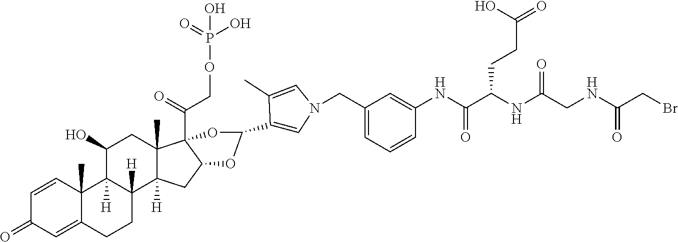 |
| II-D-48 | 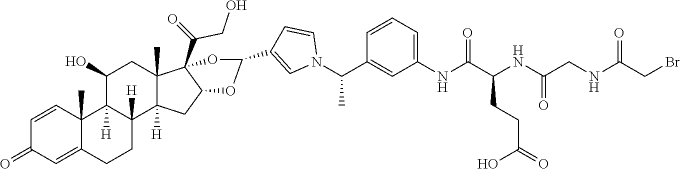 |
| II-D-49 | 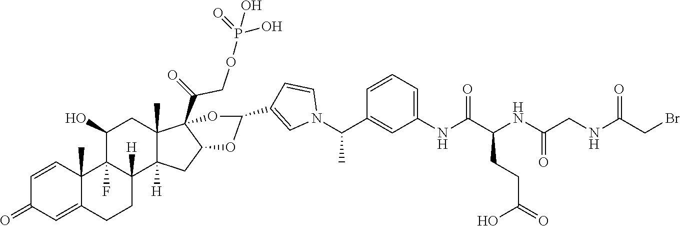 |
| II-D-53 | 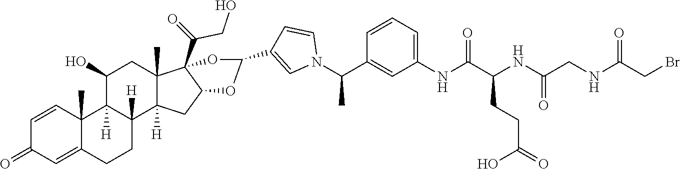 |
| II-D-54 | 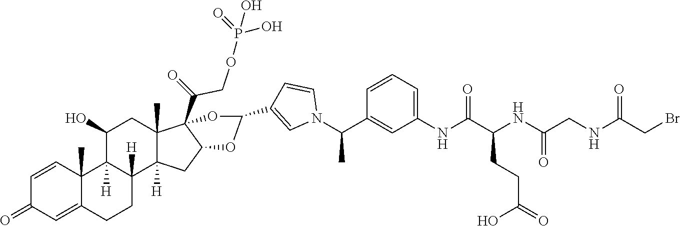 |
| II-D-78 | 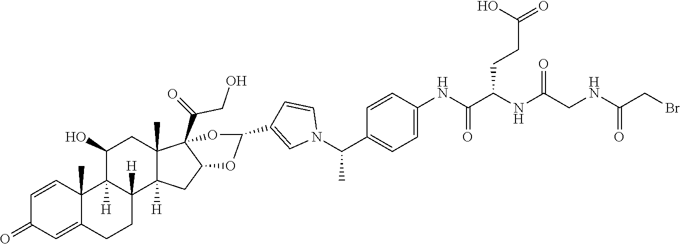 |
| II-D-79 | 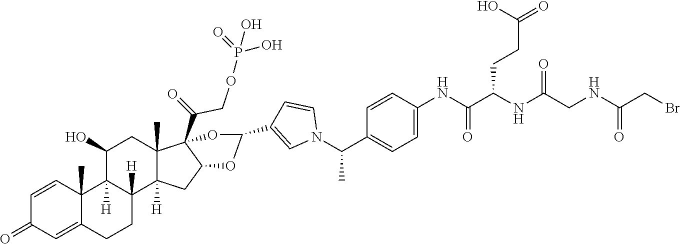 |
| II-D-92 | 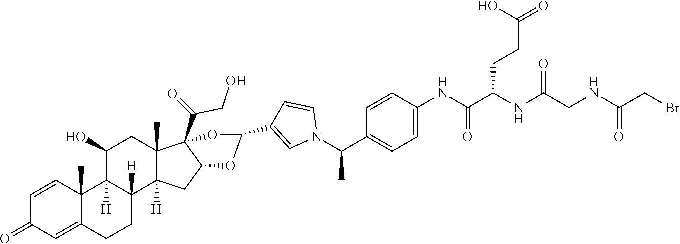 |
| II-D-93 | 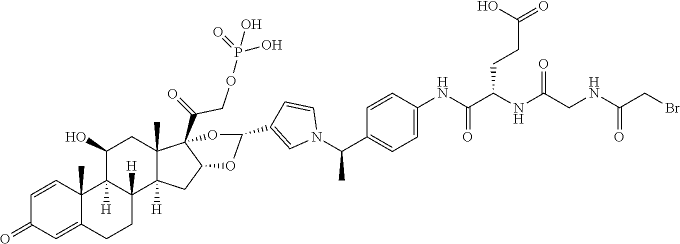 |
| II-D-111 | 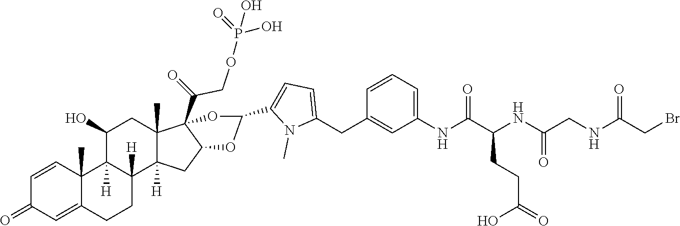 |
| II-D-114 | 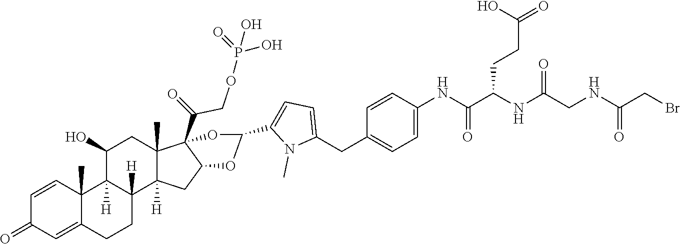 |
| II-D-117 | 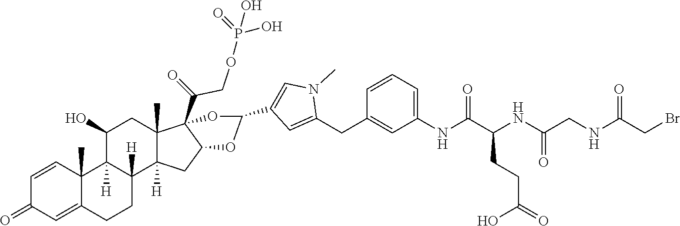 |
| II-D-120 | 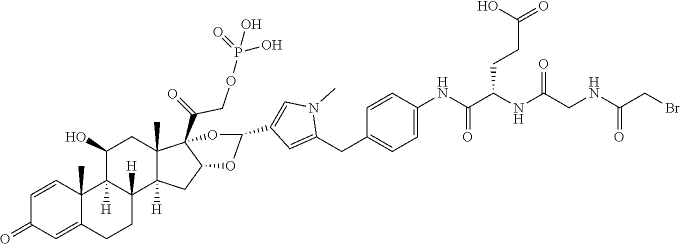 |
| II-D-123 | 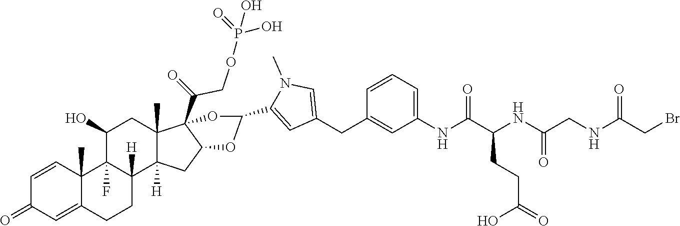 |
| II-D-126 | 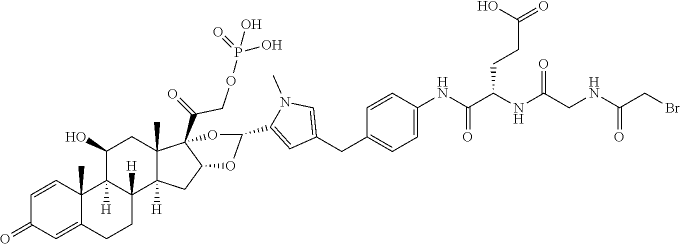 |
| II-D-170 | 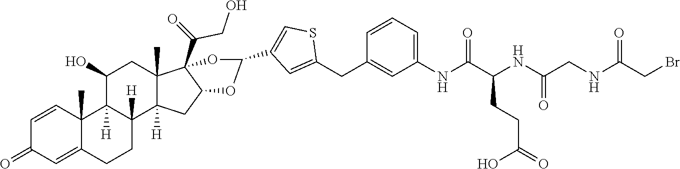 |
| II-D-171 | 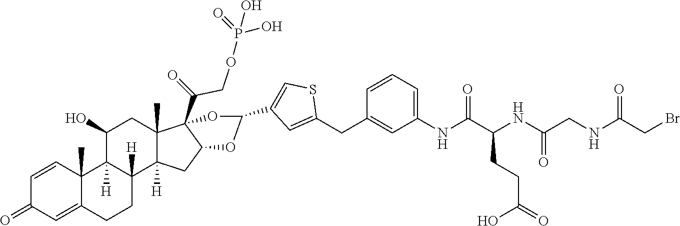 |
| II-D-175 | 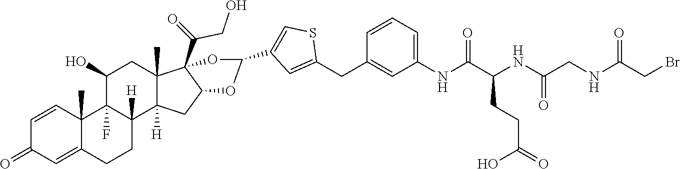 |
| II-D-176 | 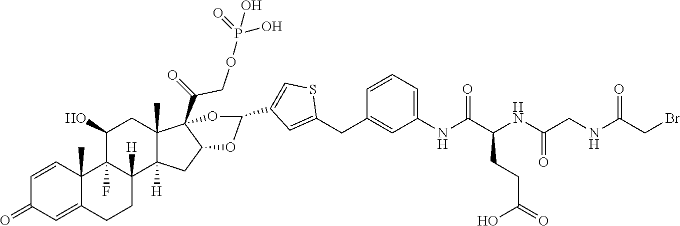 |
| II-D-180 | 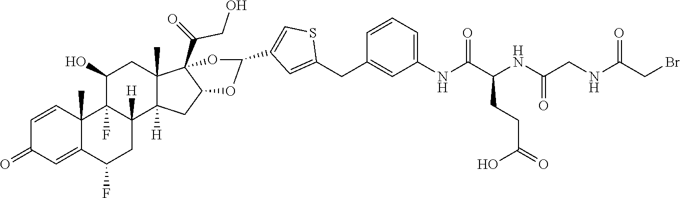 |
| II-D-181 | 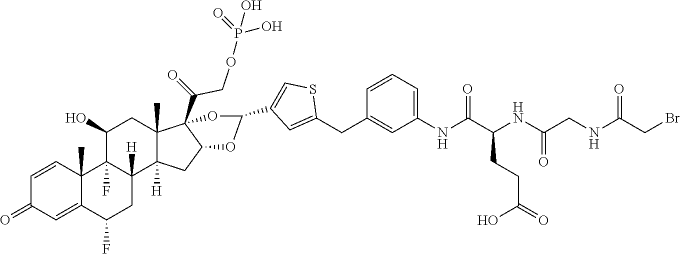 |
| II-D-185 | 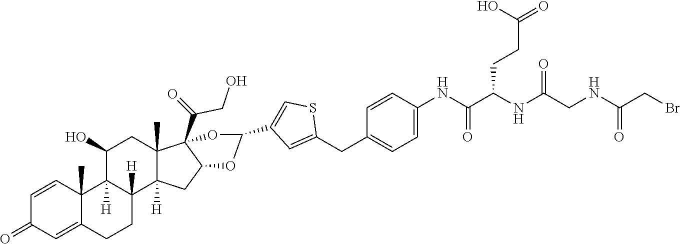 |
| II-D-186 | 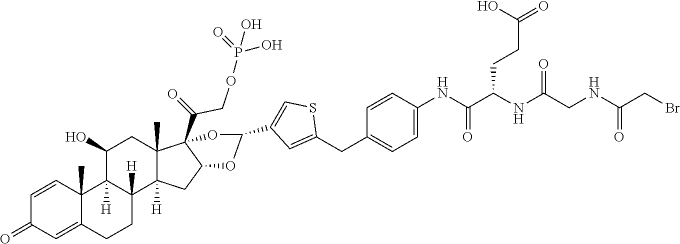 |
| II-D-190 | 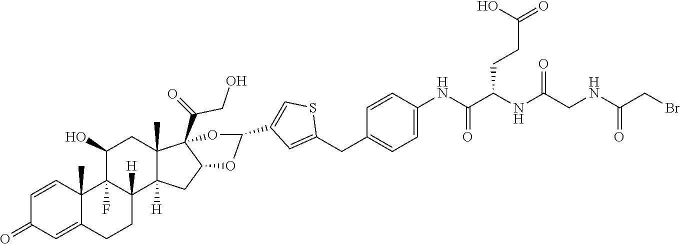 |
| II-D-191 | 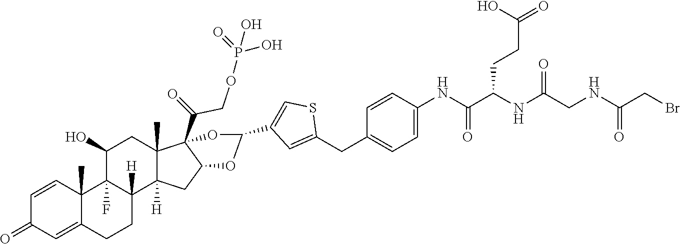 |
| II-D-195 | 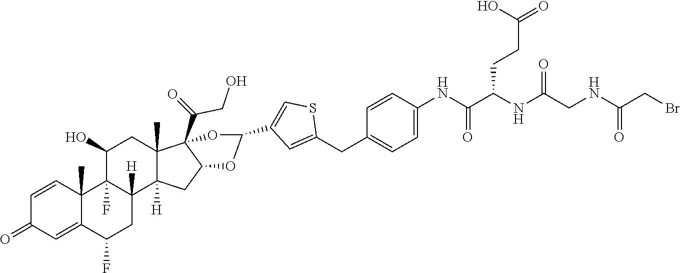 |
| II-D-196 | 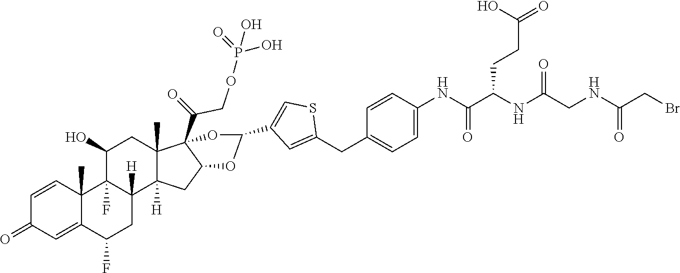 |
| II-D-200 | 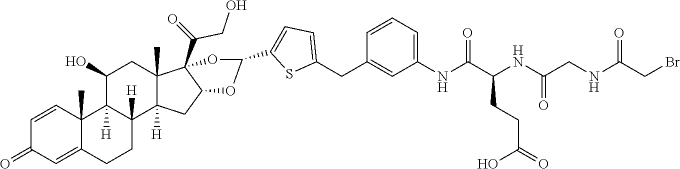 |
| II-D-201 | 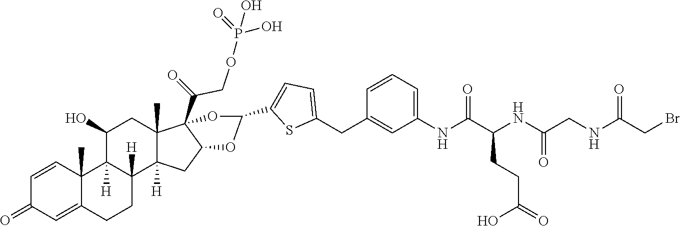 |
| II-D-205 | 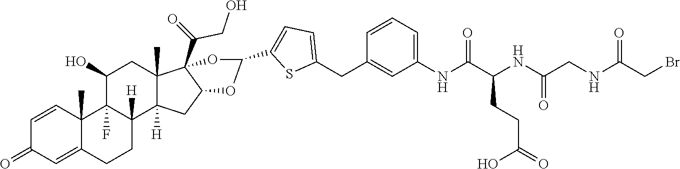 |
| II-D-206 | 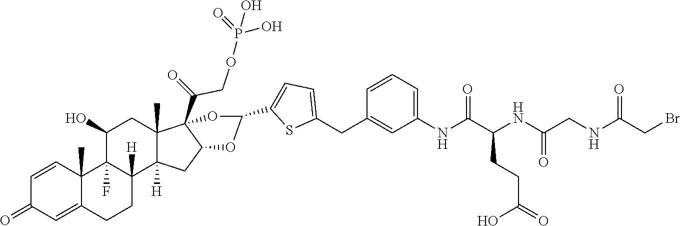 |
| II-D-210 | 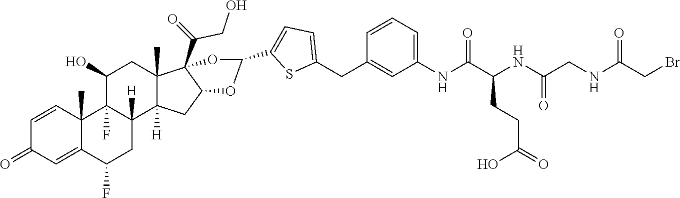 |
| II-D-211 | 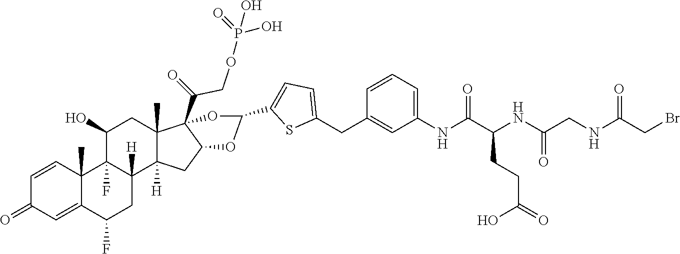 |
| II-D-215 | 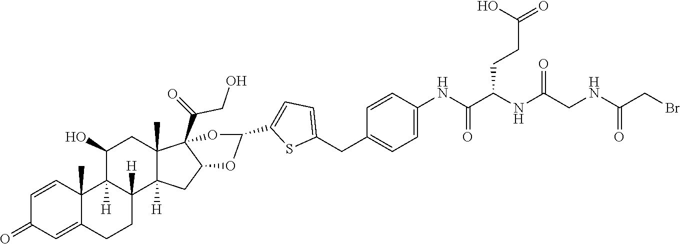 |
| II-D-216 | 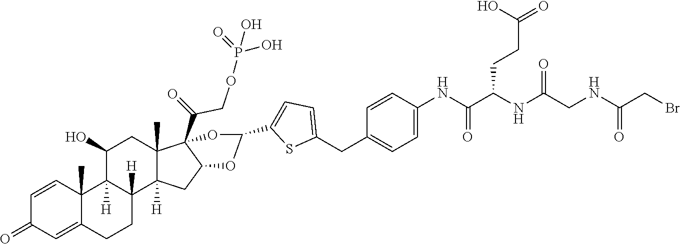 |
| II-D-220 | 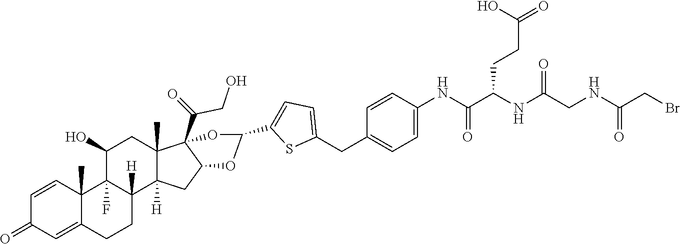 |
| II-D-221 | 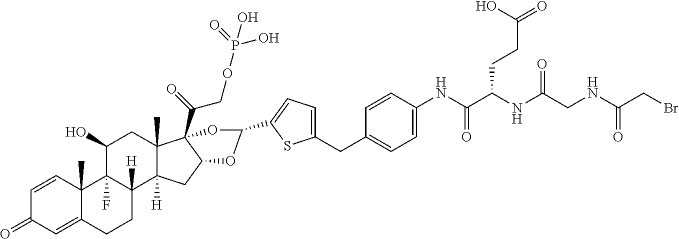 |
| II-D-225 | 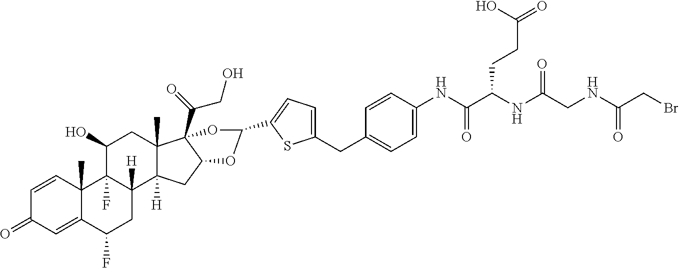 |
| II-D-226 | 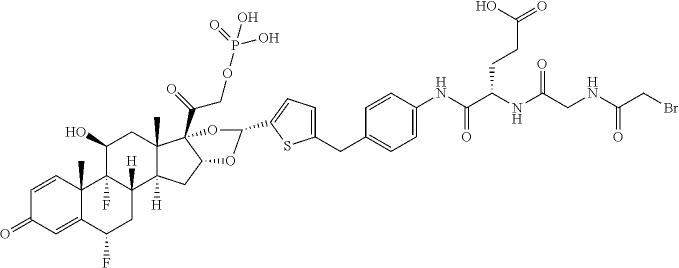 |
| II-D-238 | 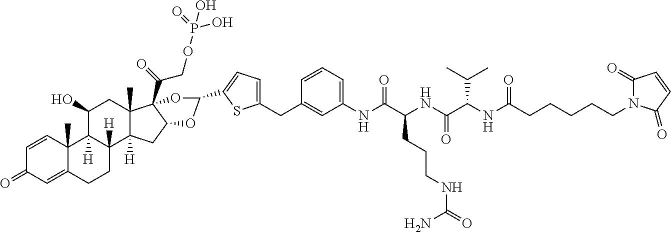 |
| II-D-239 | 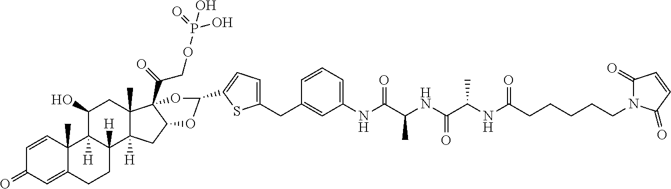 |
| II-D-240 | 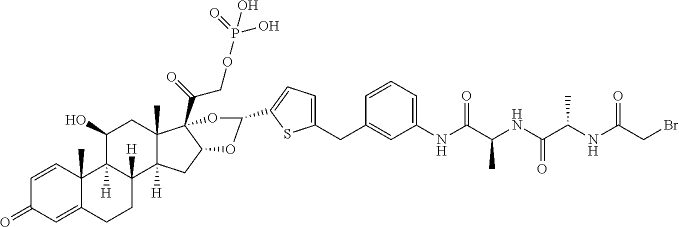 |
| II-D-241 | 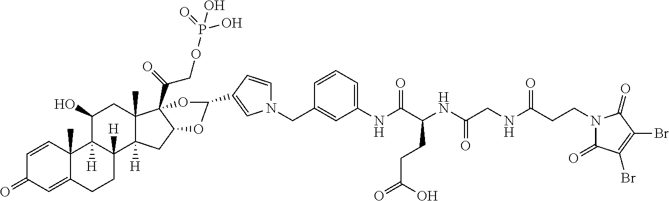 |
| II-D-244 | 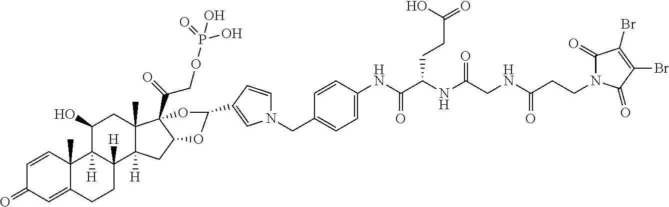 |
| II-D-247 | 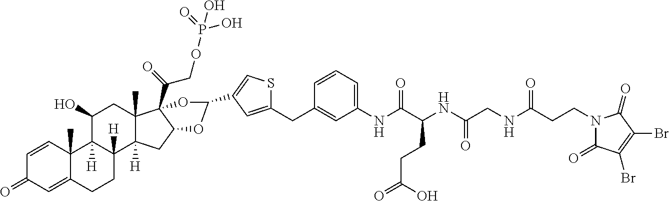 |
| II-D-248 | 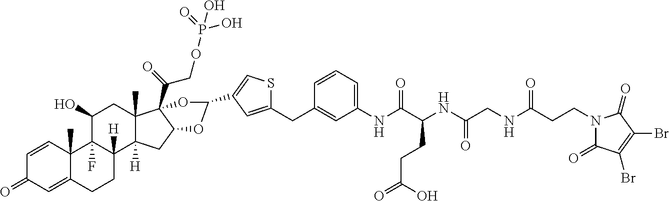 |
| II-D-249 | 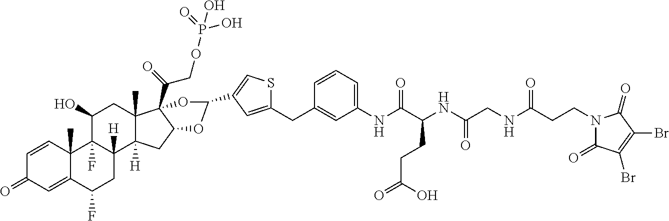 |
| II-D-250 | 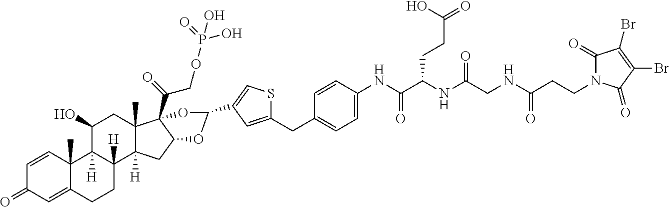 |
| II-D-251 | 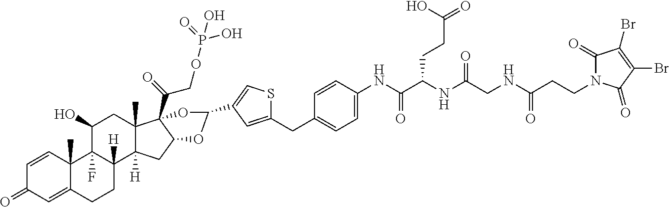 |
| II-D-252 | 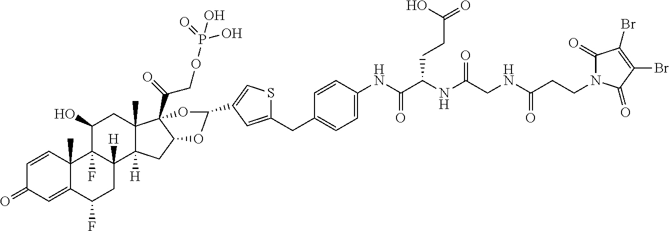 |
| II-D-253 | 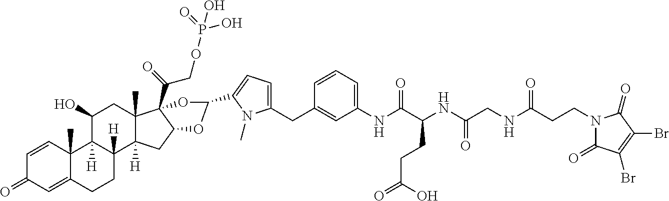 |
| II-D-256 | 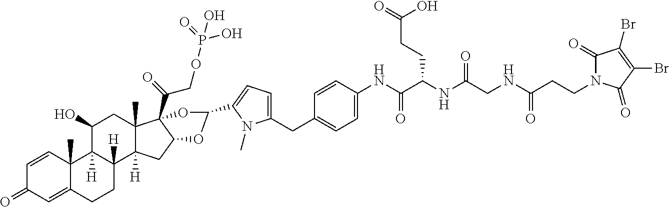 |
| II-D-259 | 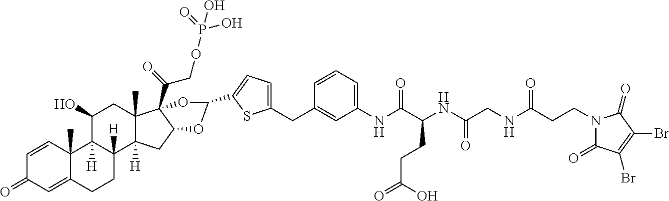 |
| II-D-260 | 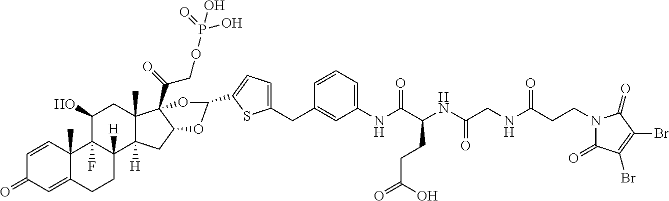 |
| II-D-261 | 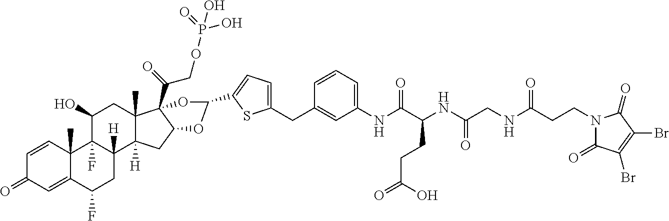 |
| II-D-262 | 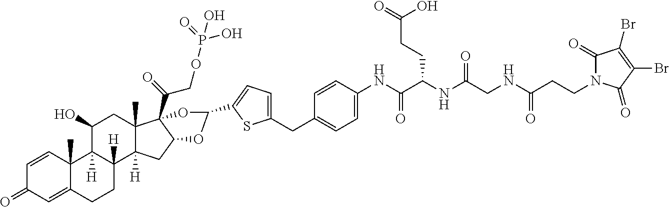 |
| II-D-263 | 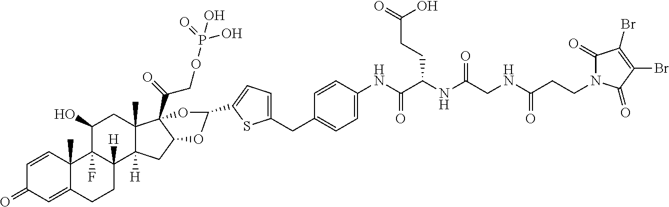 |
| II-D-264 | 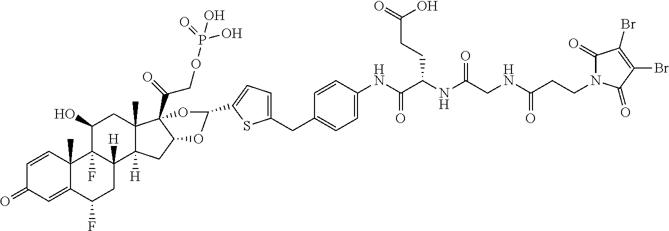 |
| II-D-265 | 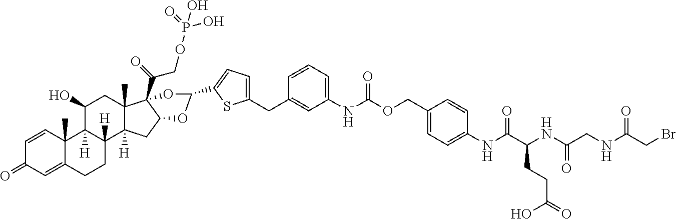 |
| II-D-266 | 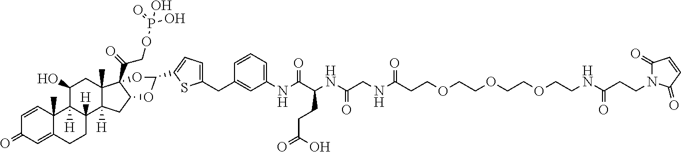 |
| II-D-267 | 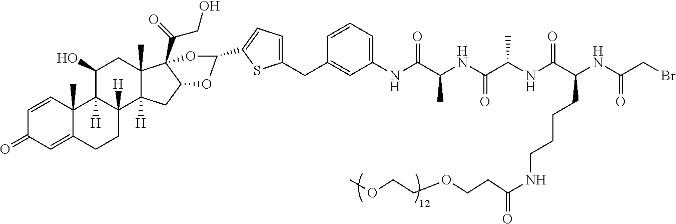 |
| II-D-268 | 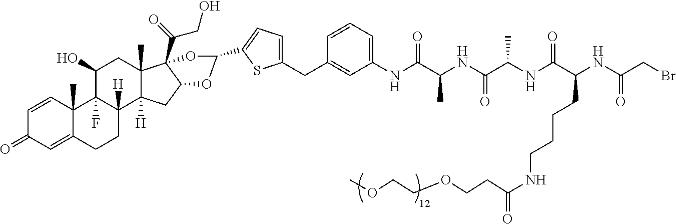 |
| II-D-269 | 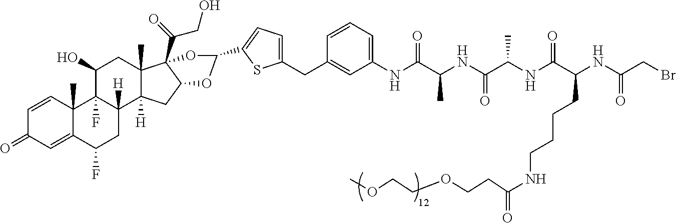 |
| II-D-270 | 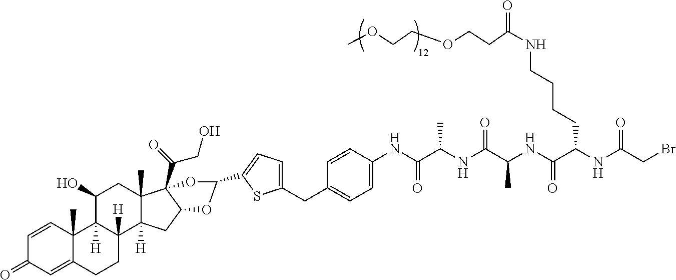 |
| II-D-271 | 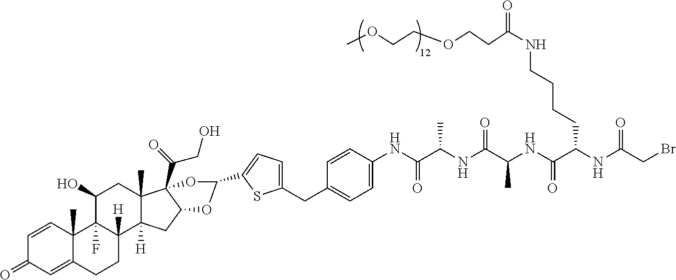 |
| II-D-272 | 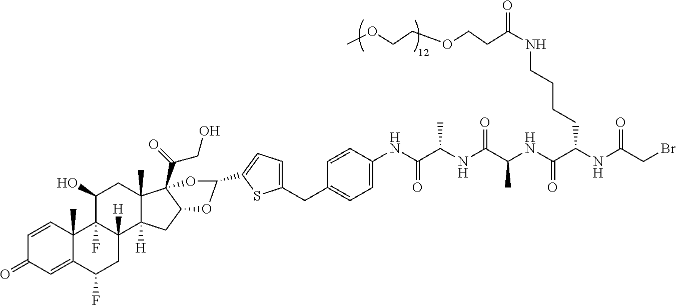 |
| II-D-273 | 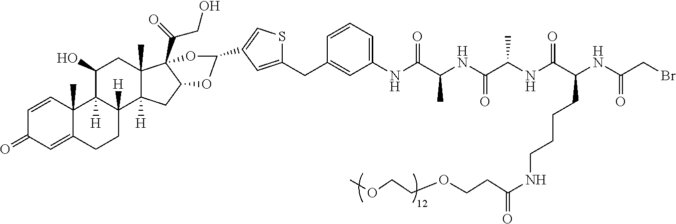 |
| II-D-274 | 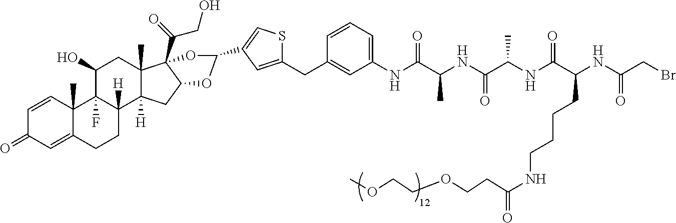 |
| II-D-275 | 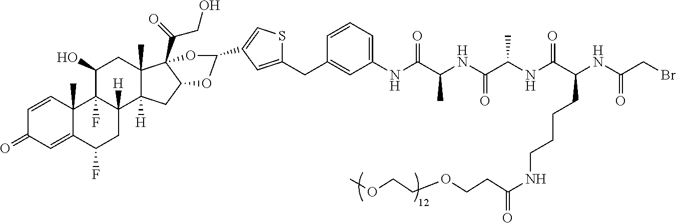 |
| II-D-276 | 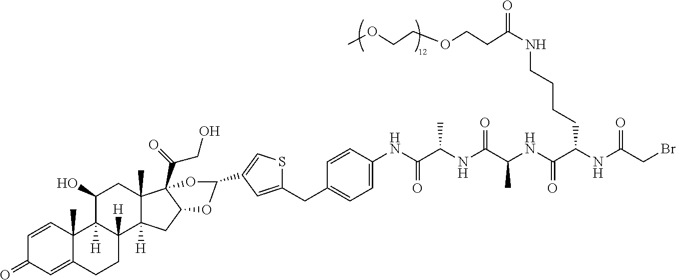 |
| II-D-277 | 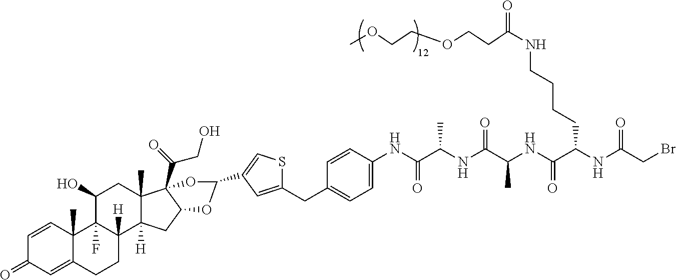 |
| II-D-278 | 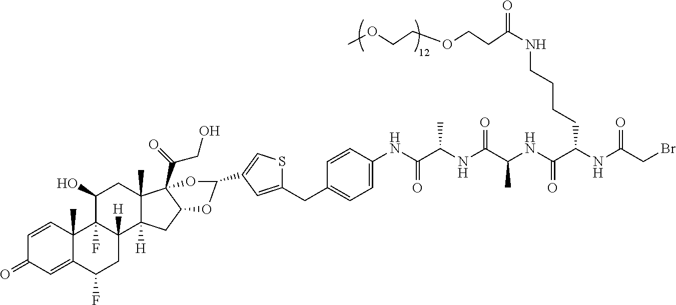 |
| II-D-279 | 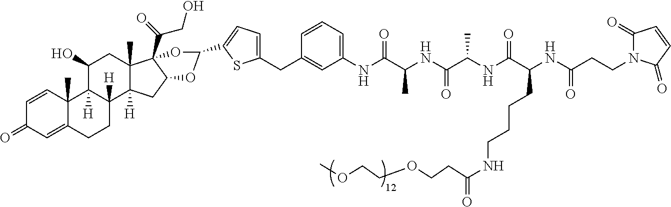 |
| II-D-280 | 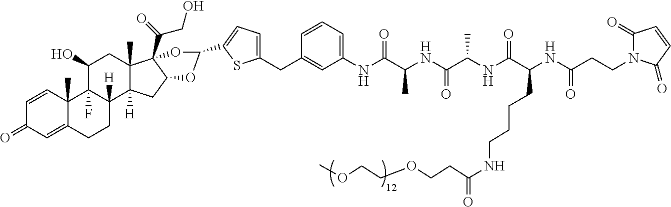 |
| II-D-281 | 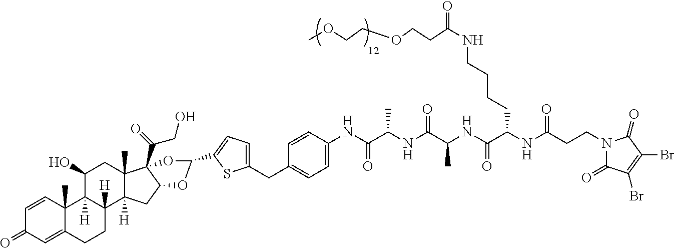 |
| II-D-282 | 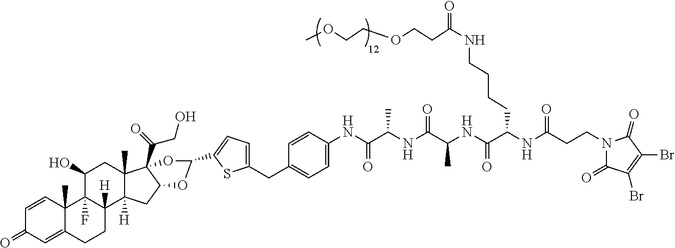 |
| II-D-283 | 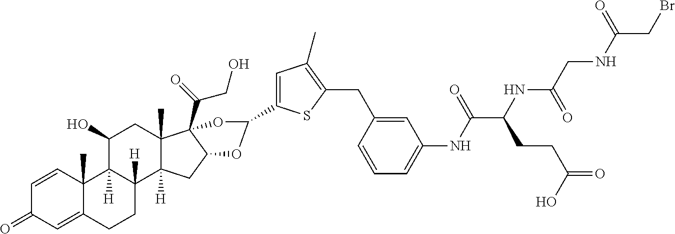 |
| II-D-286 | 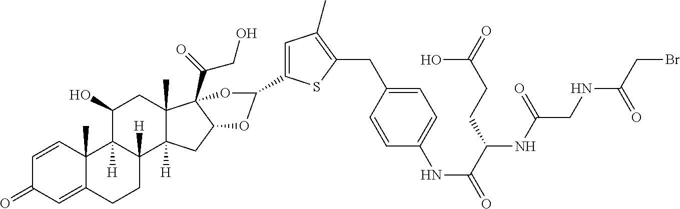 |
| II-D-289 | 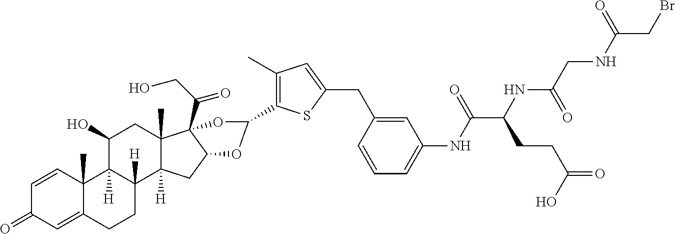 |
| II-D-292 | 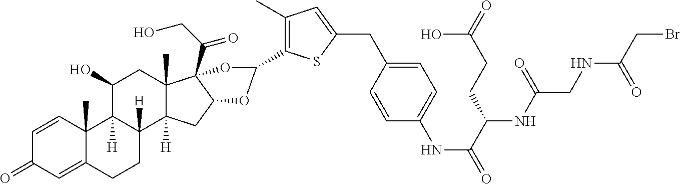 |
| II-D-295 | 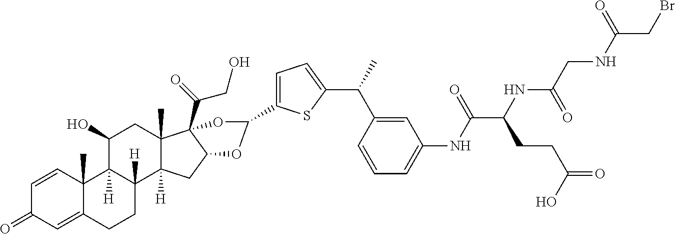 |
| II-D-298 | 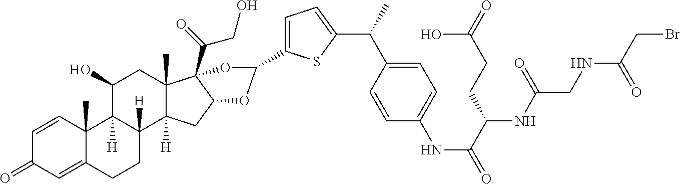 |
| II-D-301 | 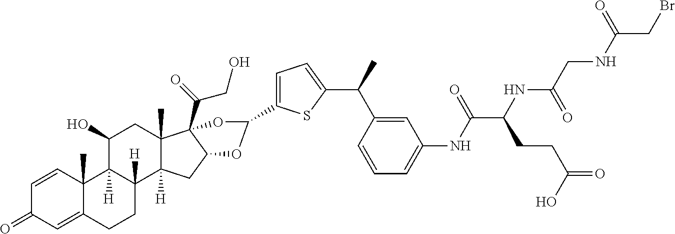 |
| II-D-304 | 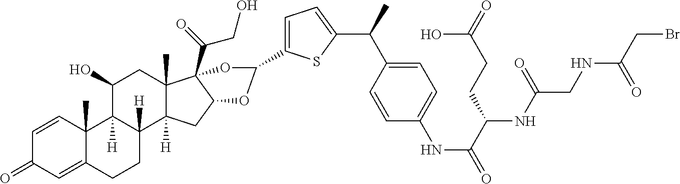 |
| II-D-307 | 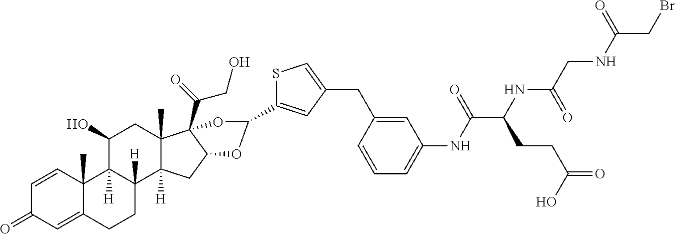 |
| II-D-310 | 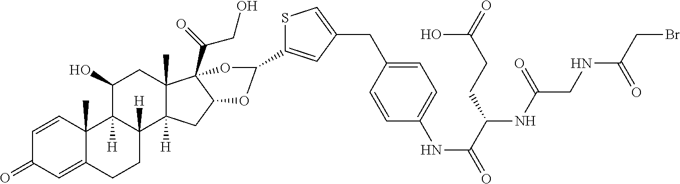 |
| II-D-313 | 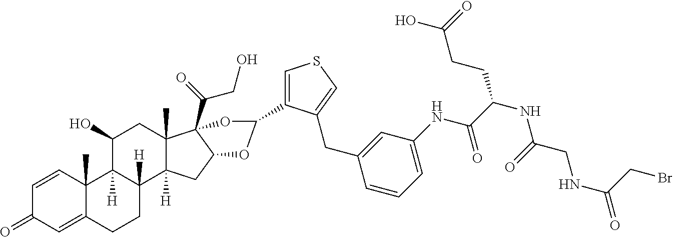 |
| II-D-314 | 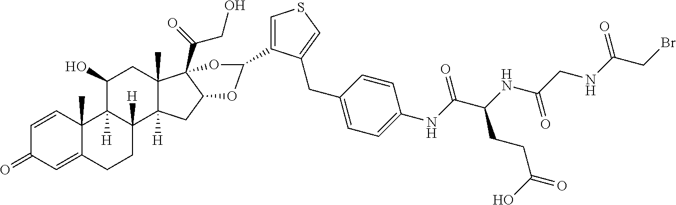 |
| II-D-315 | 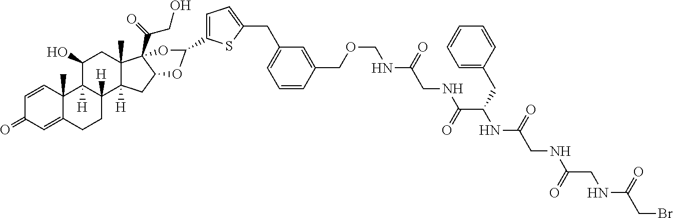 |
| II-D-316 | 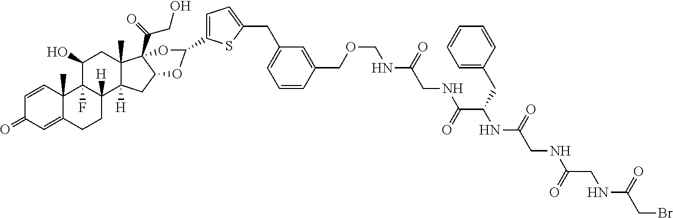 |
| II-D-317 | 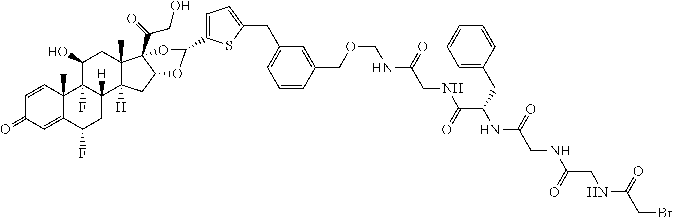 |
| II-D-318 | 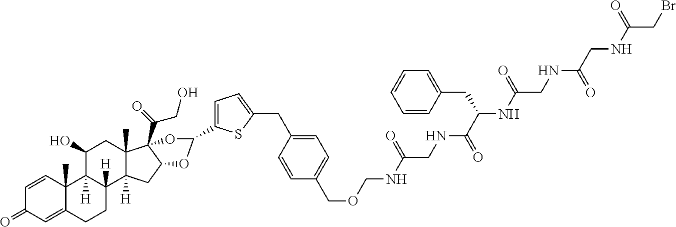 |
| II-D-319 | 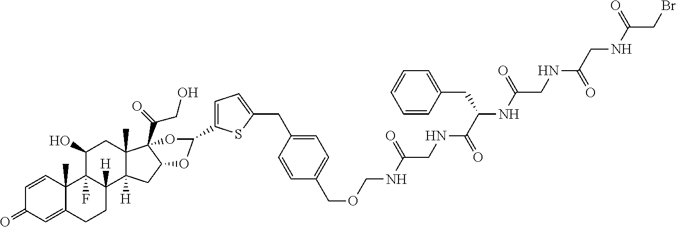 |
| II-D-320 | 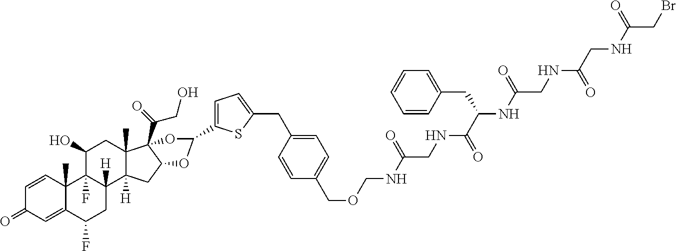 |
| II-D-321 | 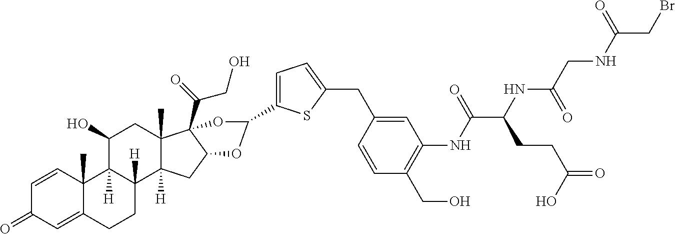 |
| II-D-322 | 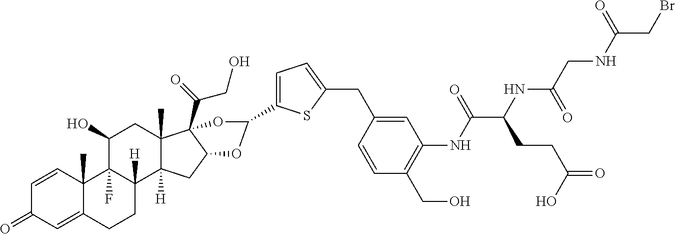 |
| II-D-323 | 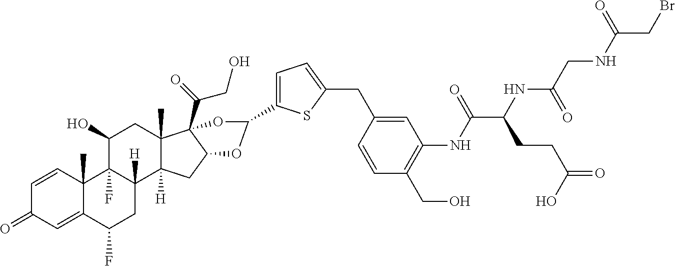 |
| II-D-324 | 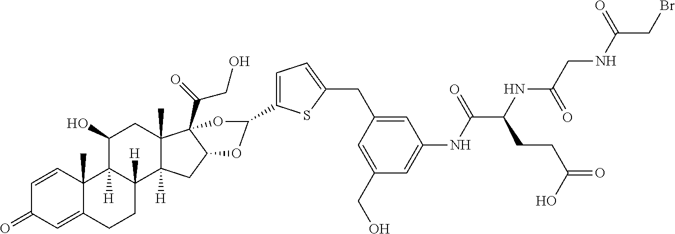 |
| II-D-325 | 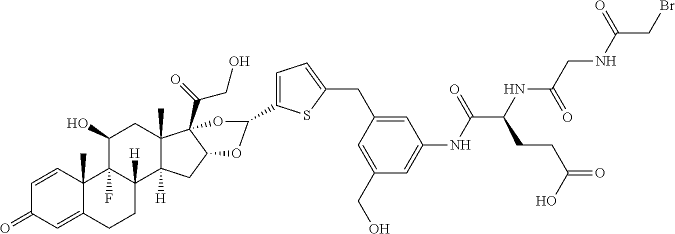 |
| II-D-326 | 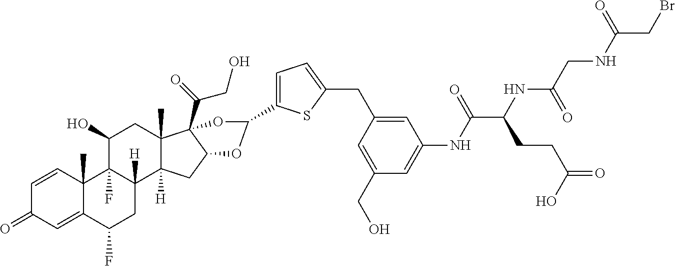 |
| II-D-327 | 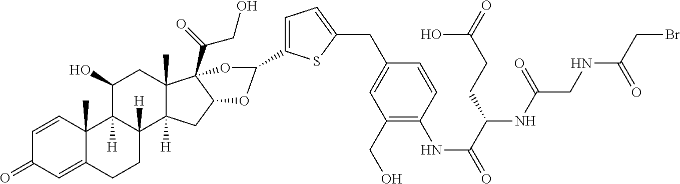 |
| II-D-328 | 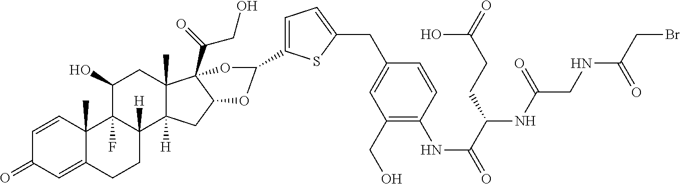 |
| II-D-329 | 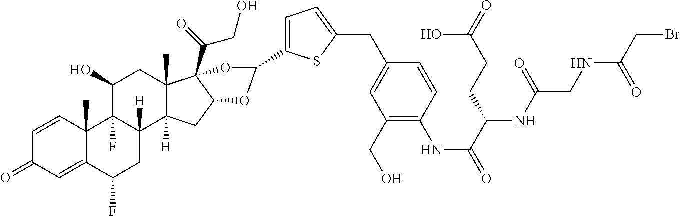 |
| II-D-330 | 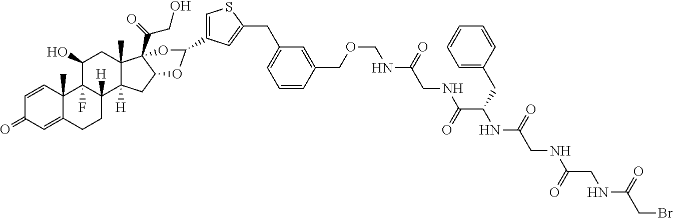 |
| II-D-331 | 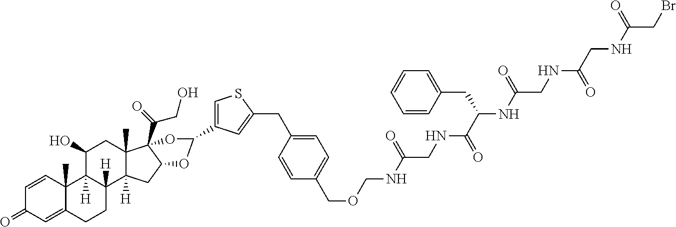 |
| II-D-332 | 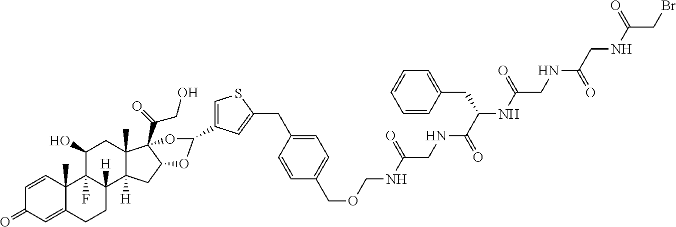 |
| II-D-333 | 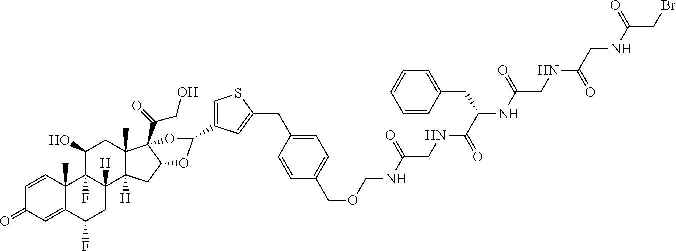 |
| II-D-334 | 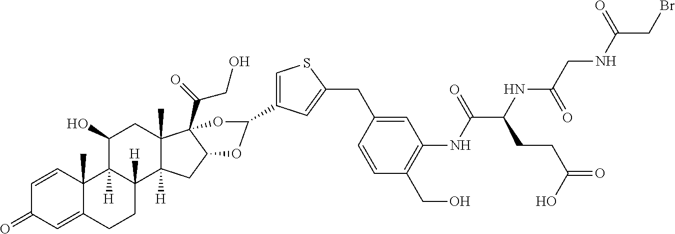 |
| II-D-335 | 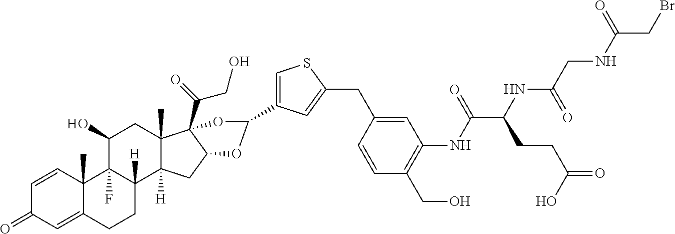 |
| II-D-336 | 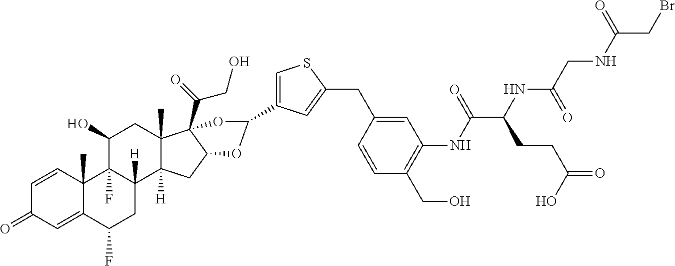 |
| II-D-337 | 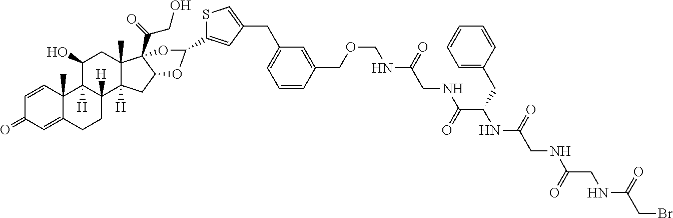 |
| II-D-338 | 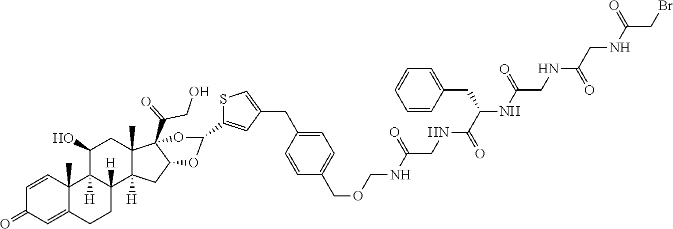 |
| II-D-339 | 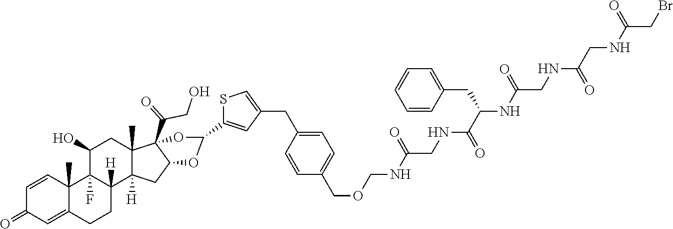 |
| II-D-340 | 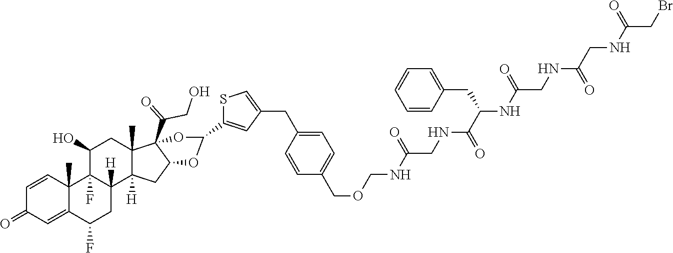 |
| II-D-341 | 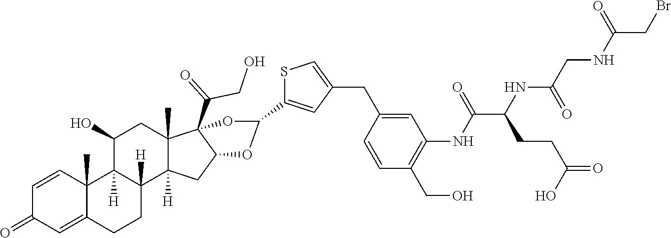 |
| II-D-342 | 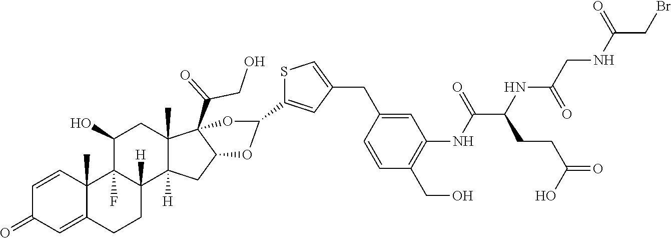 |
| II-D-343 | 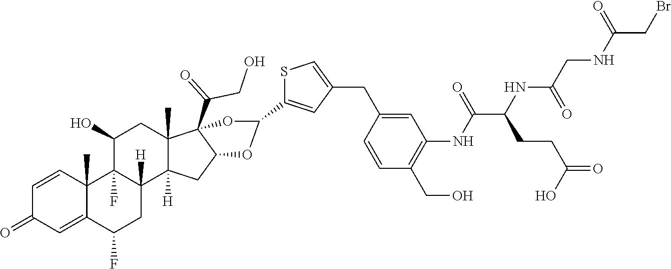 |
| II-D-344 | 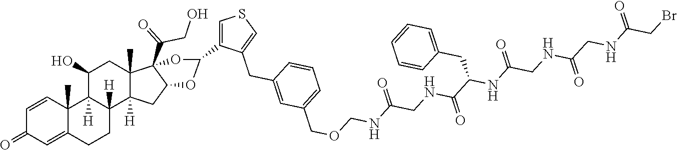 |
| II-D-345 | 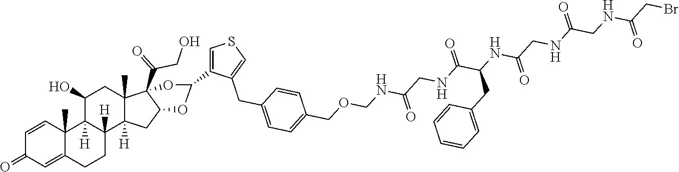 |
| II-D-361 | 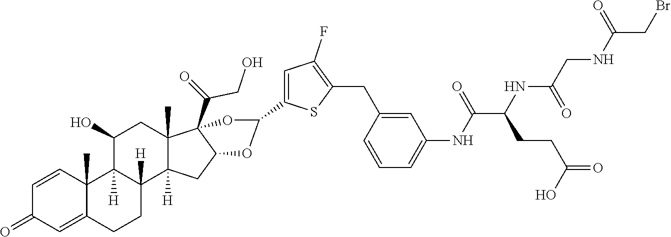 |
| II-D-364 | 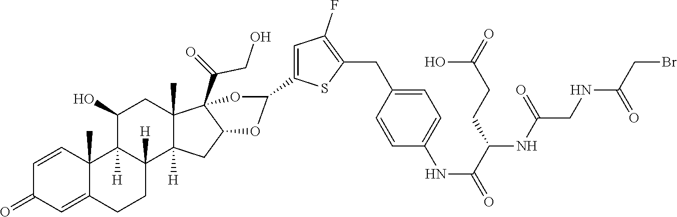 |
| II-D-367 | 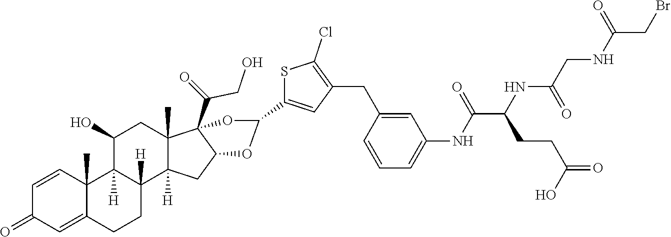 |
| II-D-370 | 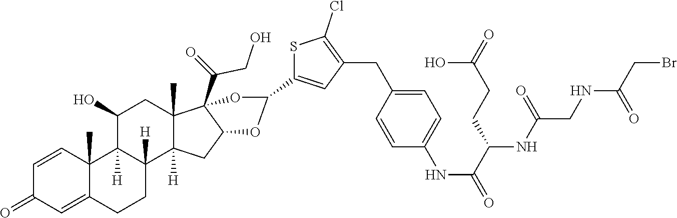 |
| II-D-373 | 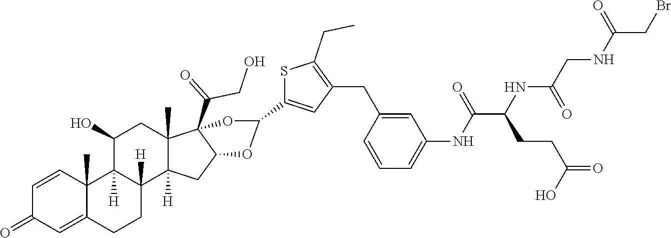 |
| II-D-376 | 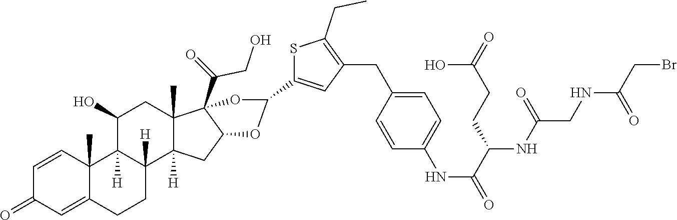 |
| II-D-379 | 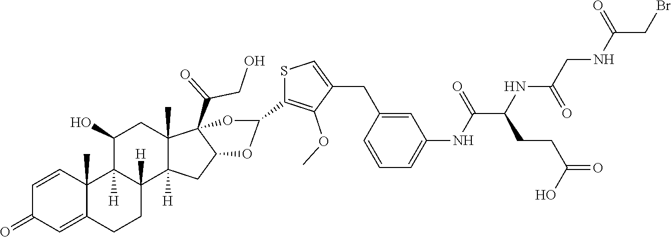 |
| II-D-382 | 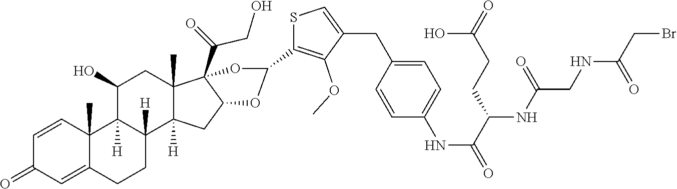 |
| II-D-385 | 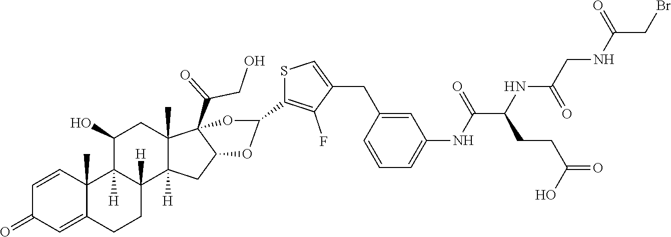 |
| II-D-388 | 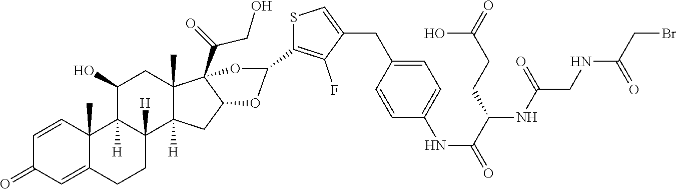 |
| II-D-391 | 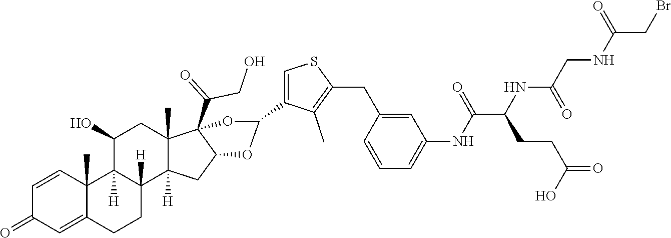 |
| II-D-394 | 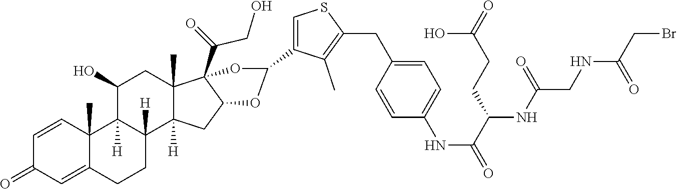 |
| II-D-397 | 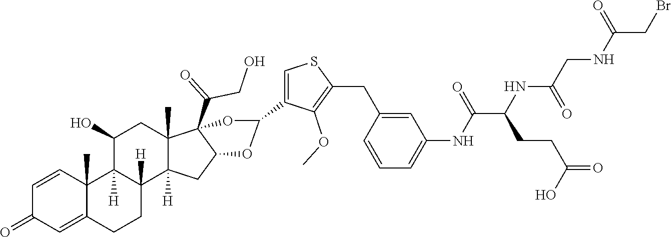 |
| II-D-400 | 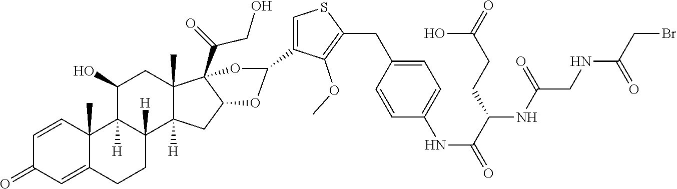 |
| II-D-403 | 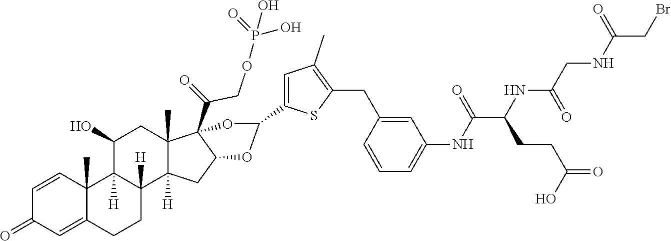 |
| II-D-406 | 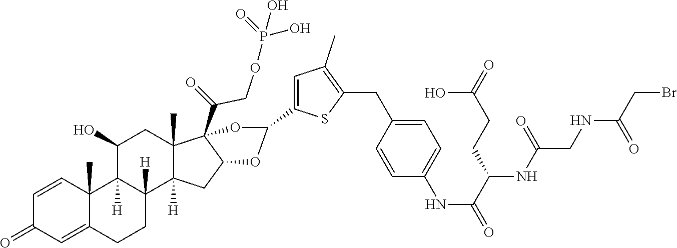 |
| II-D-409 | 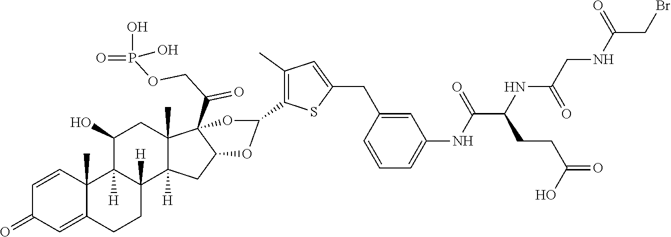 |
| II-D-412 | 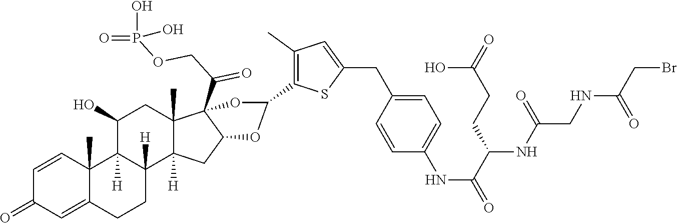 |
| II-D-415 | 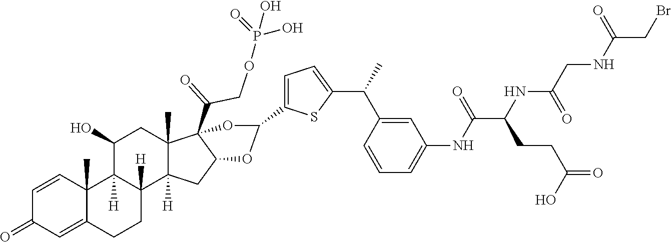 |
| II-D-418 | 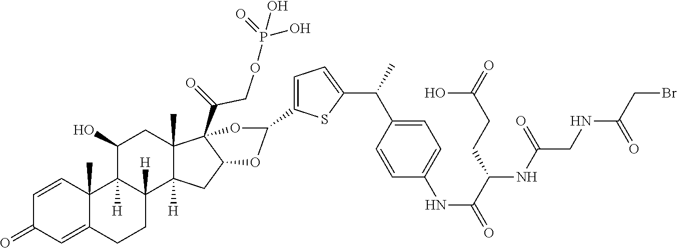 |
| II-D-421 | 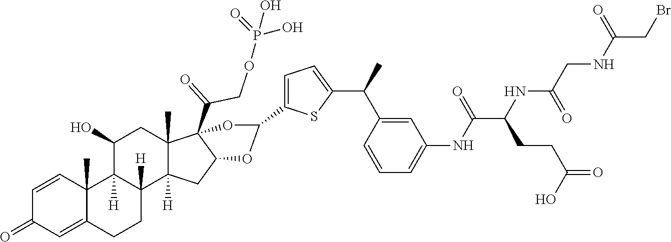 |
| II-D-424 | 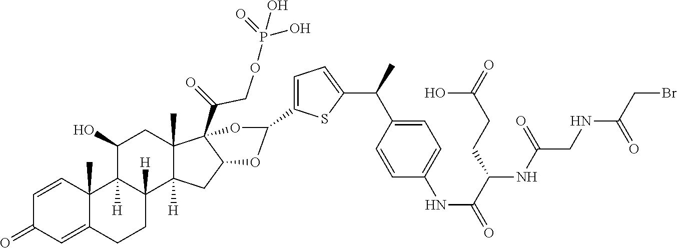 |
| II-D-427 | 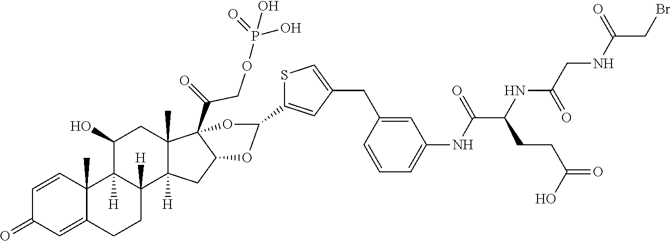 |
| II-D-439 | 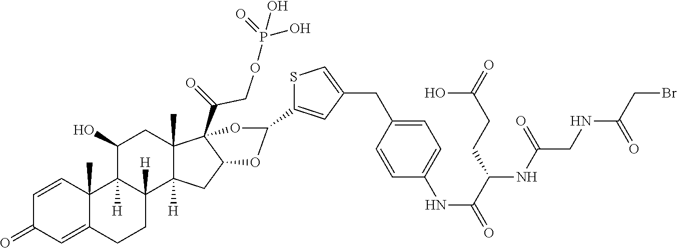 |
| II-D-451 | 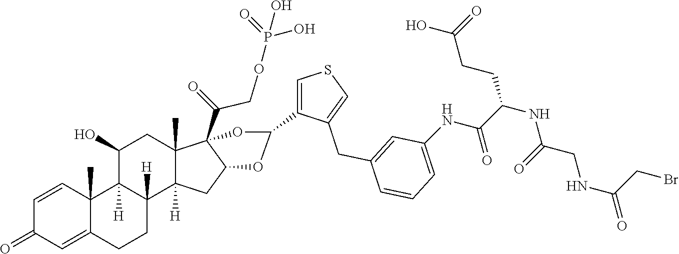 |
| II-D-452 | 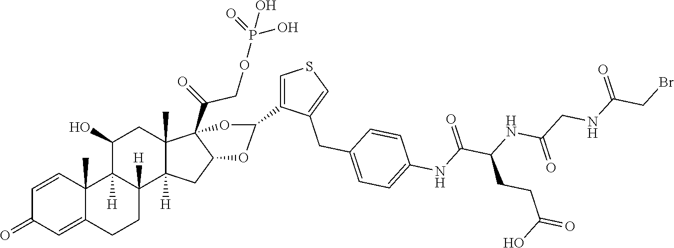 |
| II-D-453 | 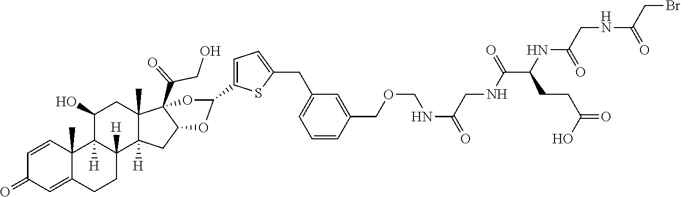 |
| II-D-454 | 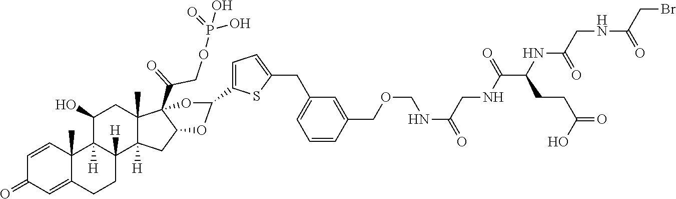 |
| II-D-455 | 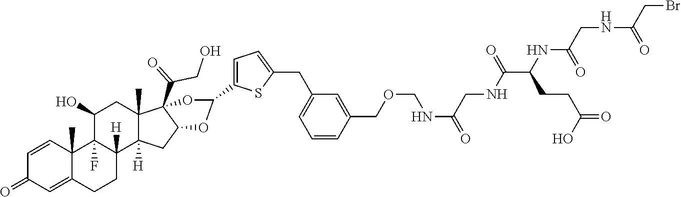 |
| II-D-456 | 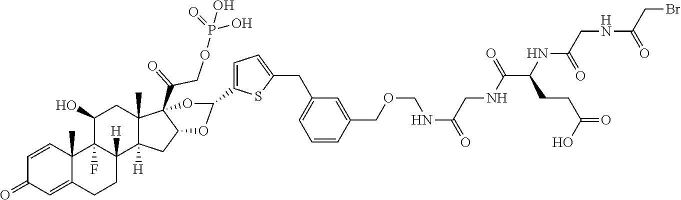 |
| II-D-457 | 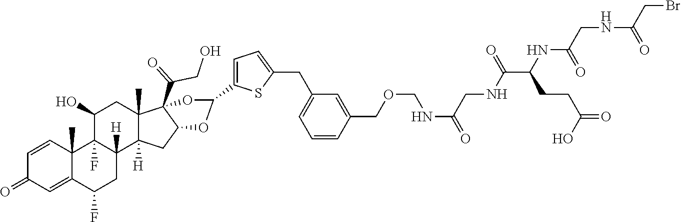 |
| II-D-458 | 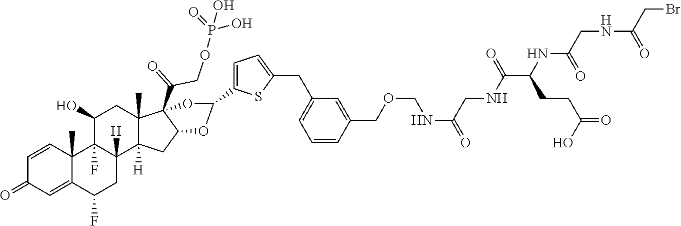 |
| II-D-459 | 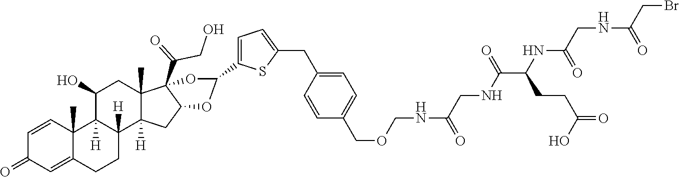 |
| II-D-460 | 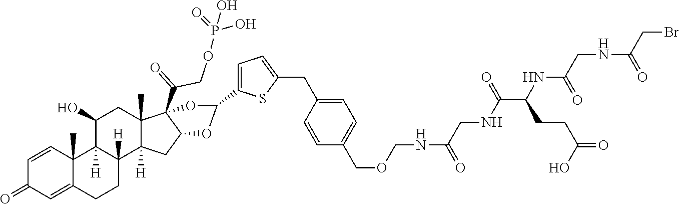 |
| II-D-461 | 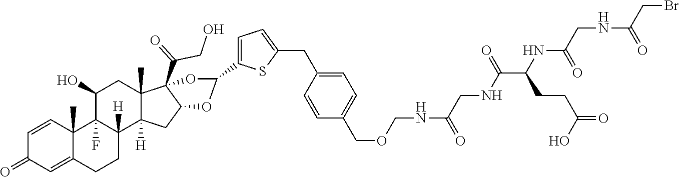 |
| II-D-462 | 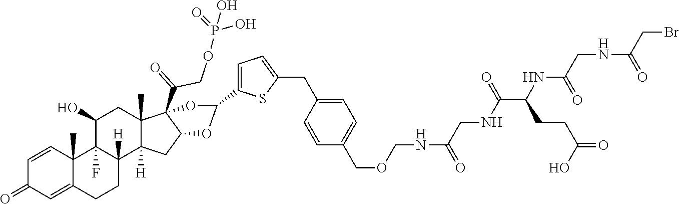 |
| II-D-463 | 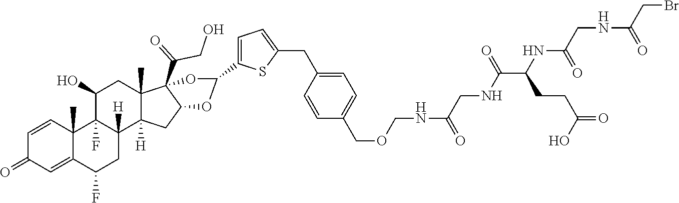 |
| II-D-464 | 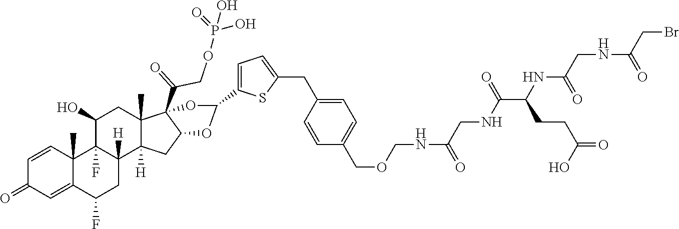 |
| II-D-465 | 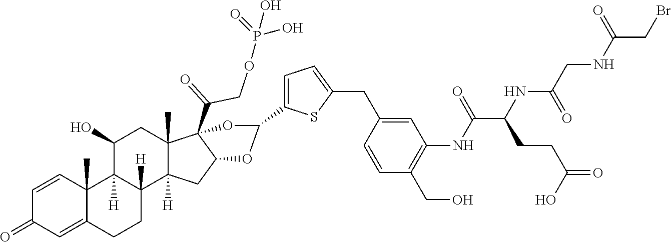 |
| II-D-468 | 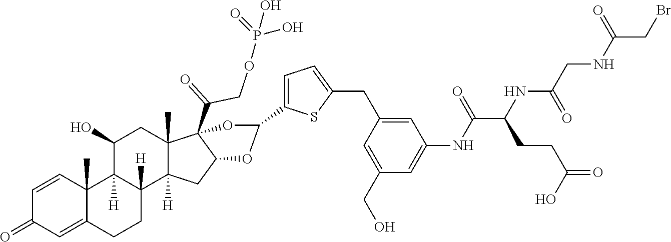 |
| II-D-471 | 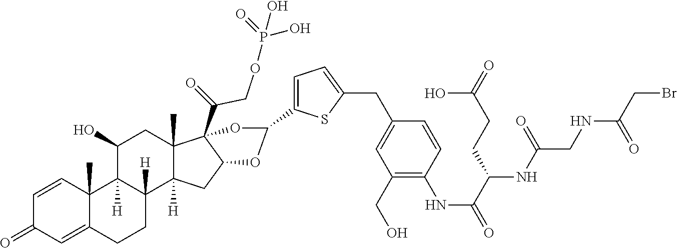 |
| II-D-474 | 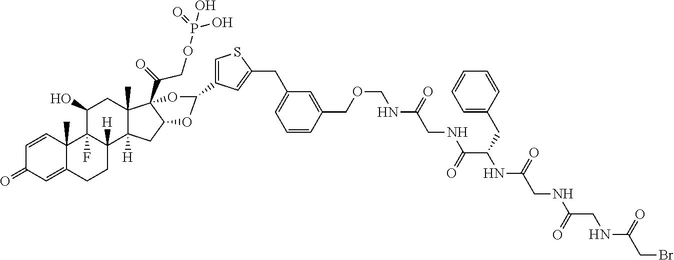 |
| II-D-475 | 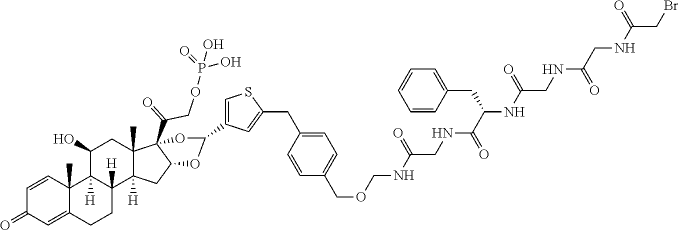 |
| II-D-476 | 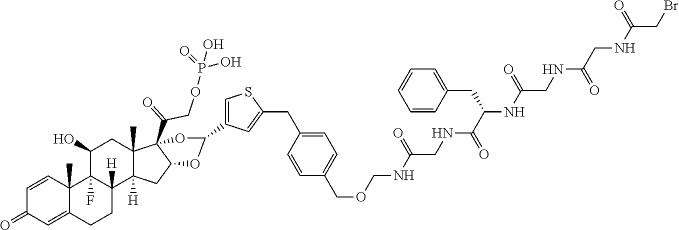 |
| II-D-477 | 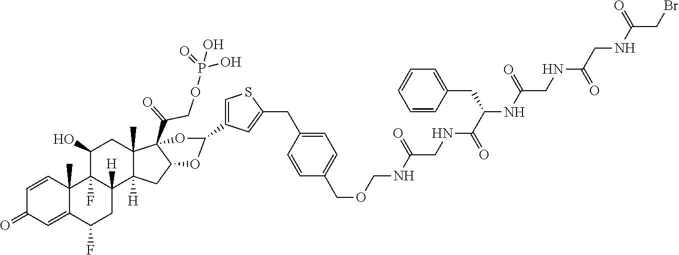 |
| II-D-478 | 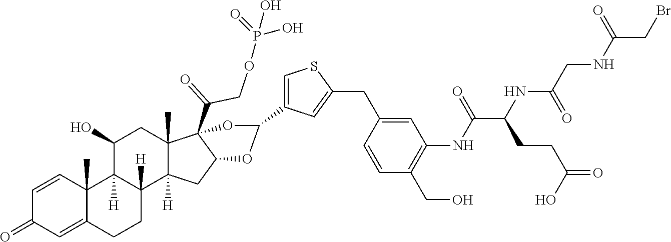 |
| II-D-481 | 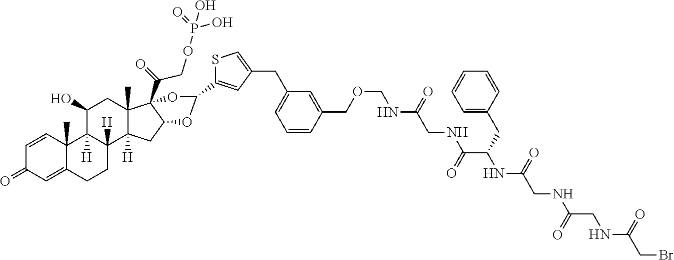 |
| II-D-482 | 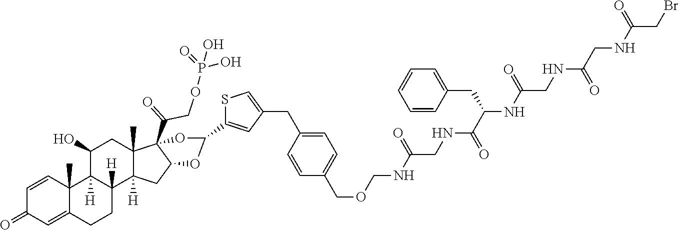 |
| II-D-483 | 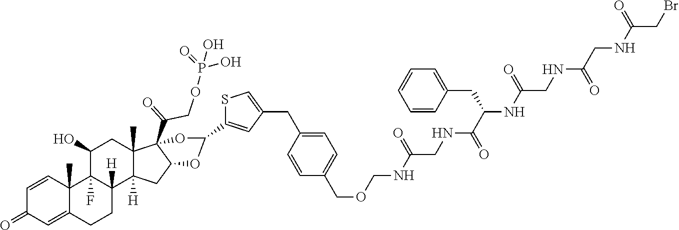 |
| II-D-484 | 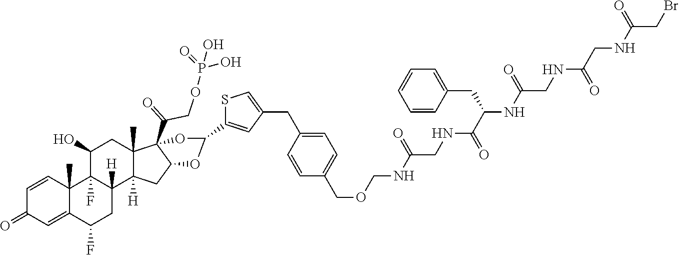 |
| II-D-485 | 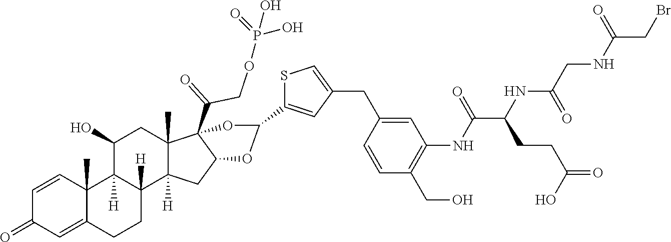 |
| II-D-488 | 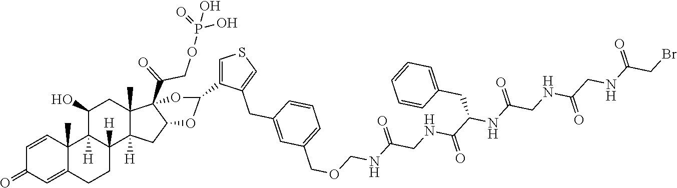 |
| II-D-489 | 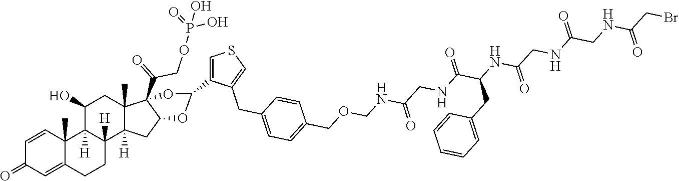 |
| II-D-505 | 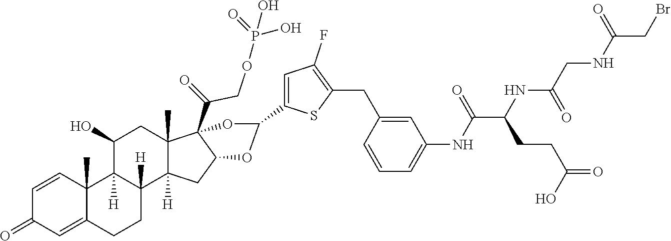 |
| II-D-508 | 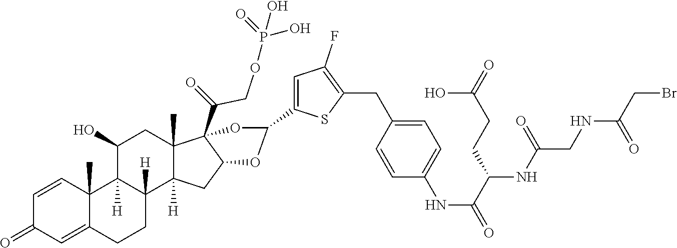 |
| II-D-511 | 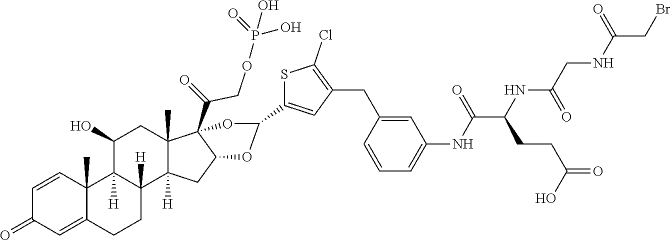 |
| II-D-514 | 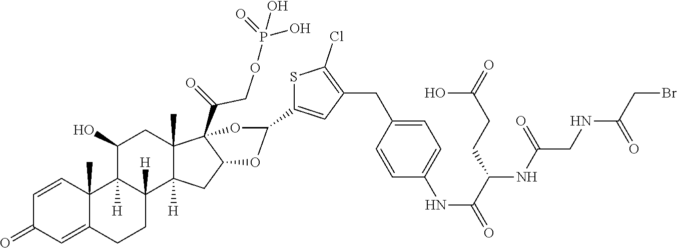 |
| II-D-517 | 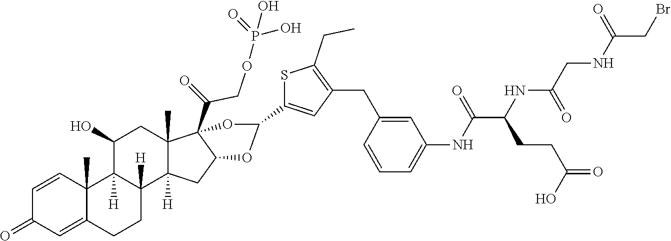 |
| II-D-520 | 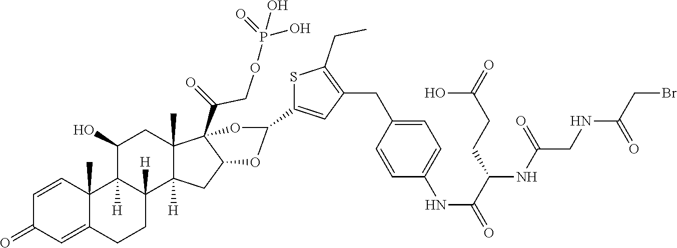 |
| II-D-523 | 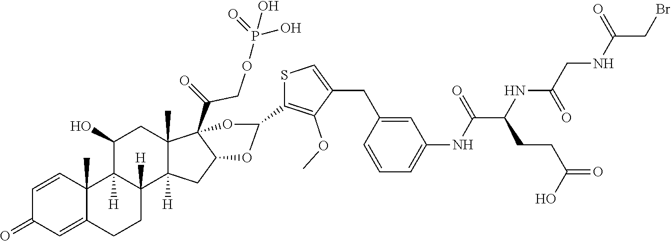 |
| II-D-526 | 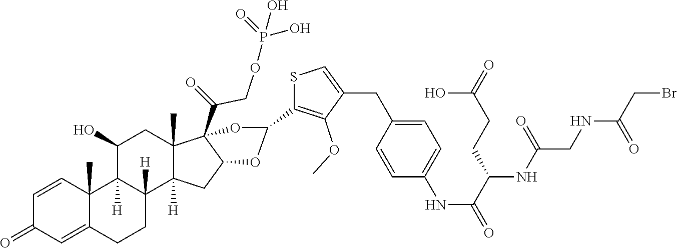 |
| II-D-529 | 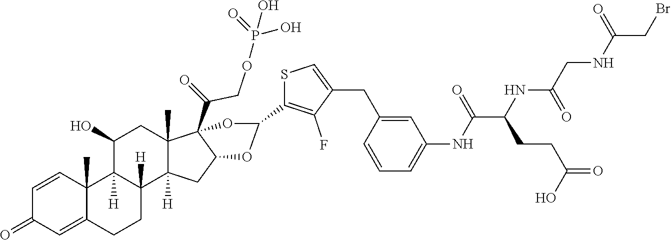 |
| II-D-532 | 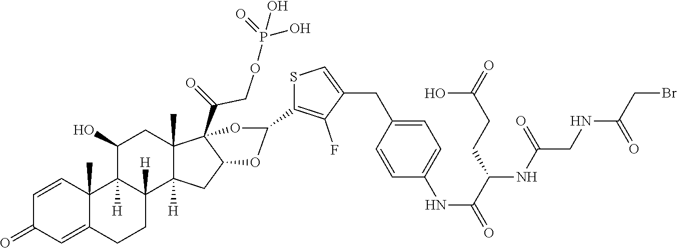 |
| II-D-535 | 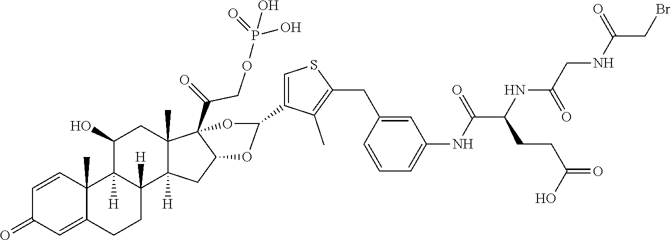 |
| II-D-538 | 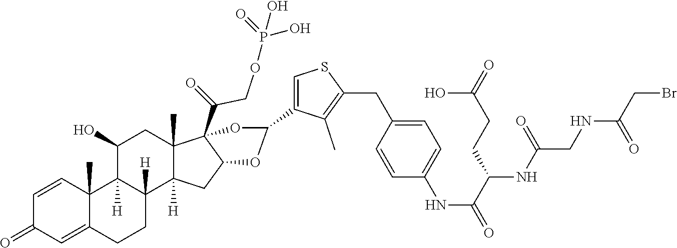 |
| II-D-541 | 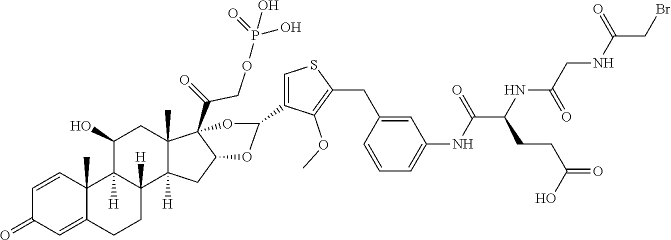 |
| II-D-544 | 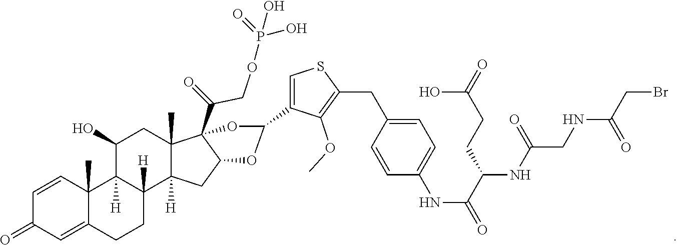 |

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group

| No. | Structure |
|---|---|
| I-C-1 | 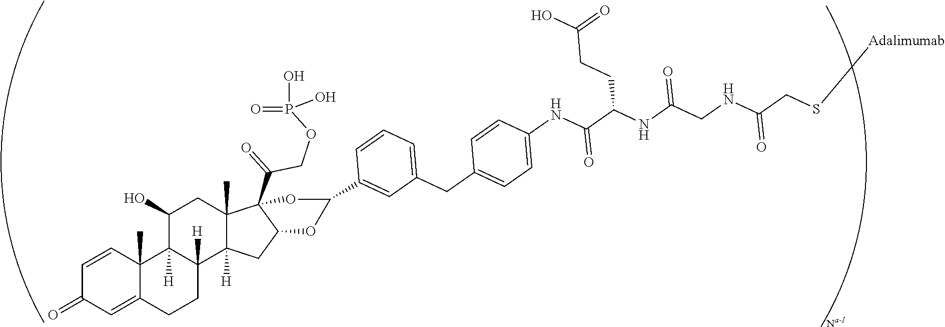 |
| I-C-2 | 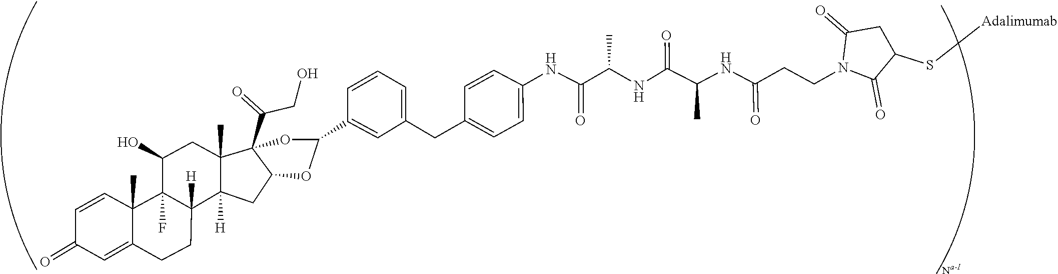 |
| I-C-3 | 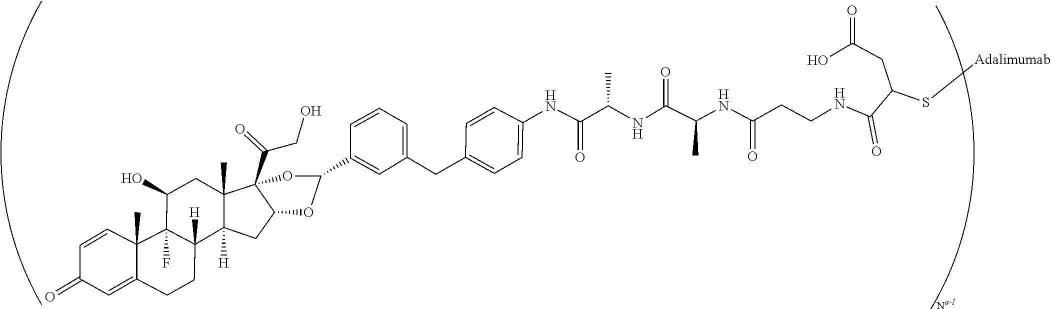 |
| I-C-4 | 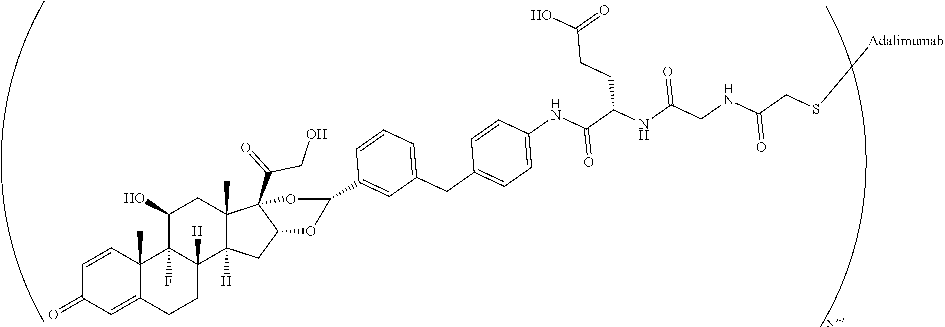 |
| I-C-5 | 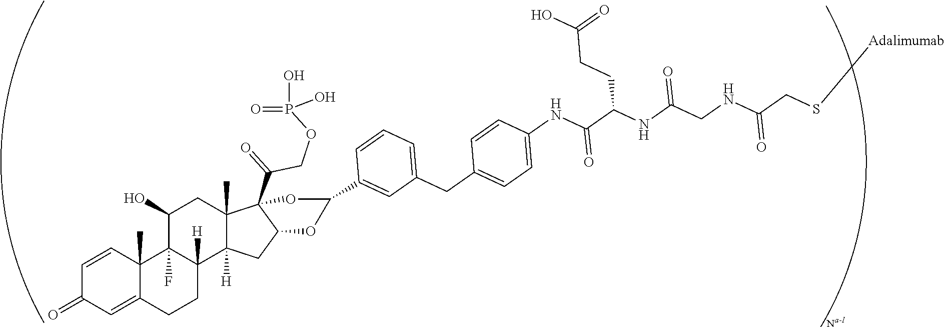 |
| I-C-6 | 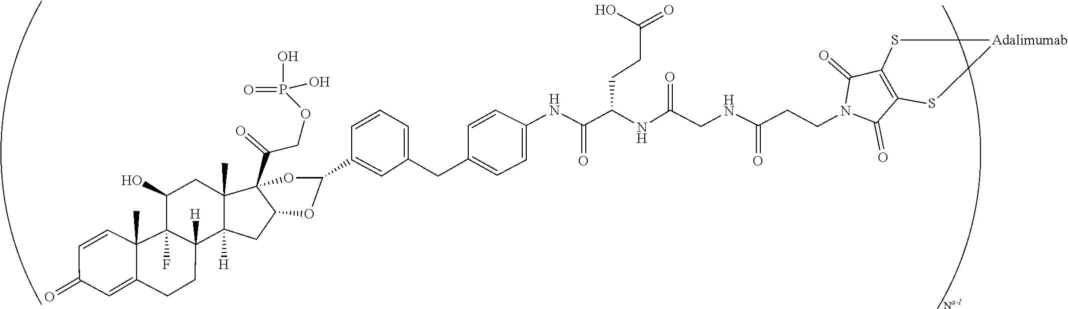 |
| I-C-11 | 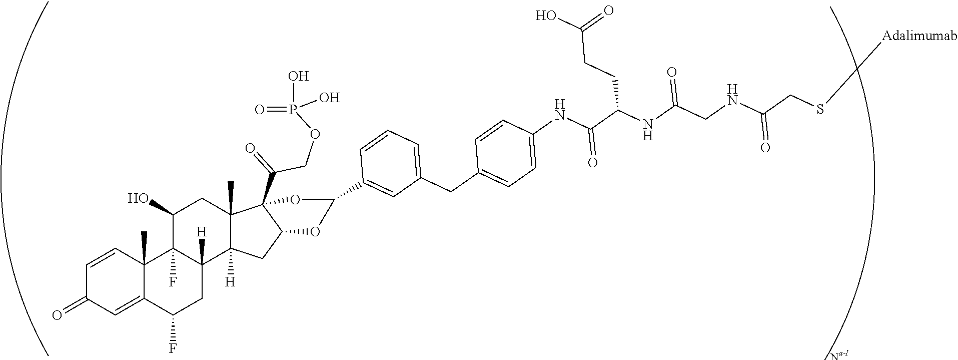 |
| I-C-16 | 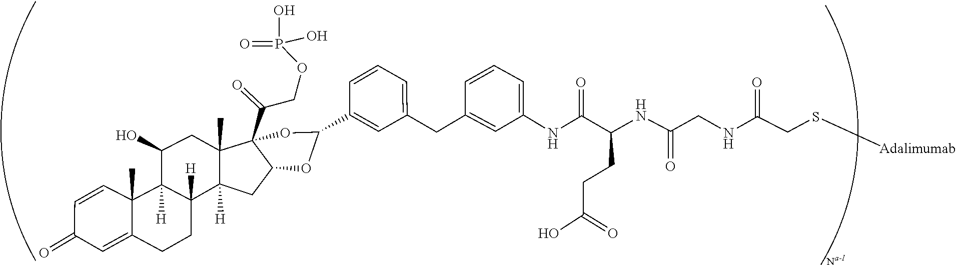 |
| I-C-17 | 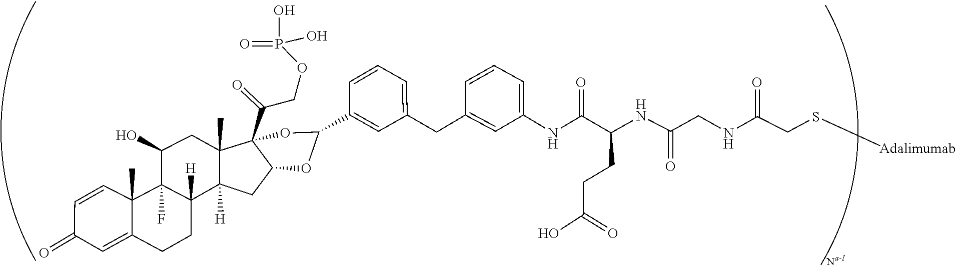 |
| I-C-18 | 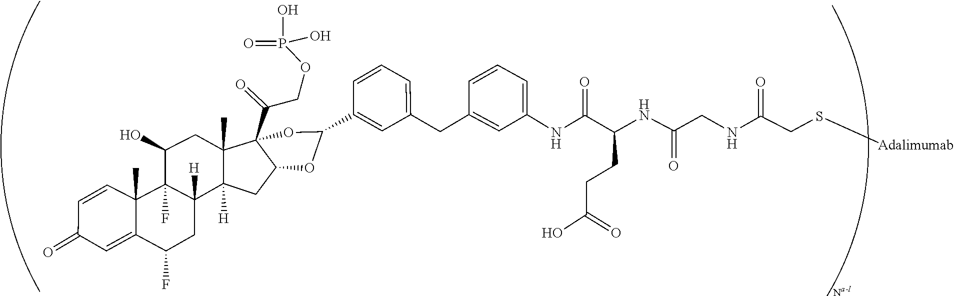 |
| I-C-19 | 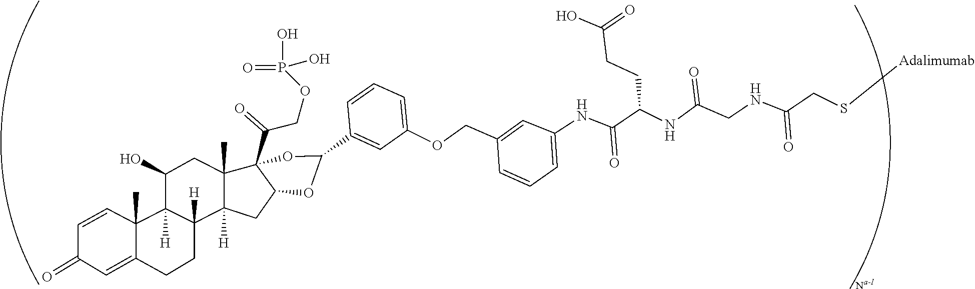 |
| I-C-21 | 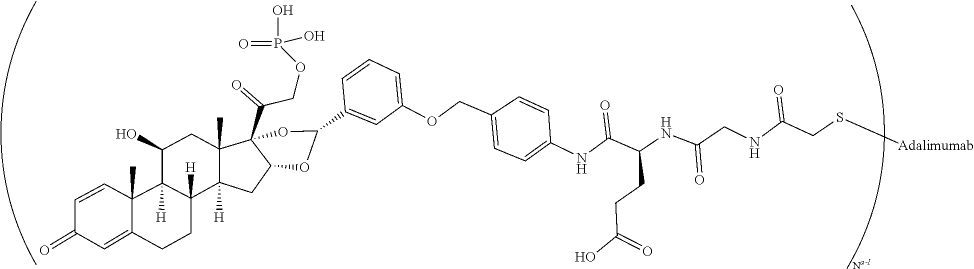 |
| I-C-33 | 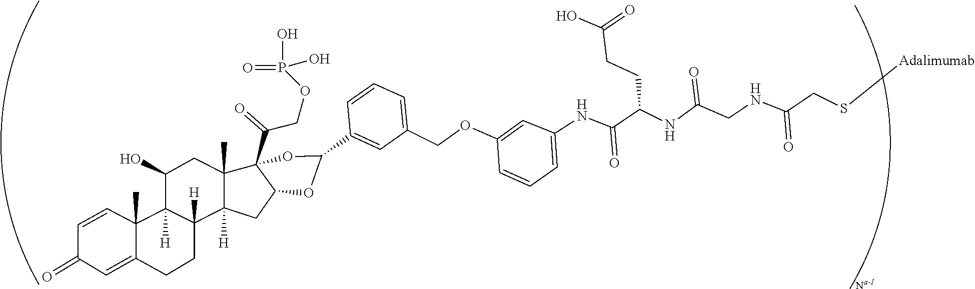 |
| I-C-35 | 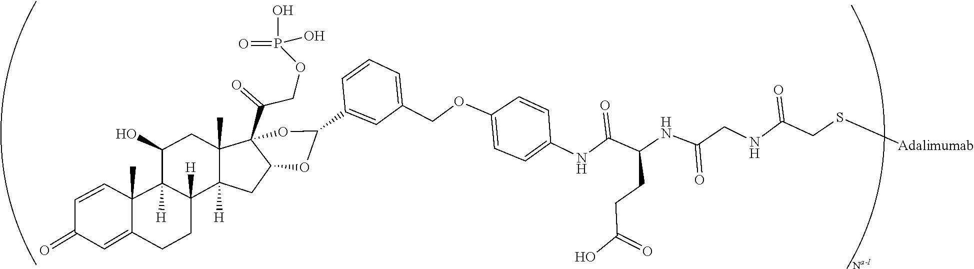 |
| I-C-47 | 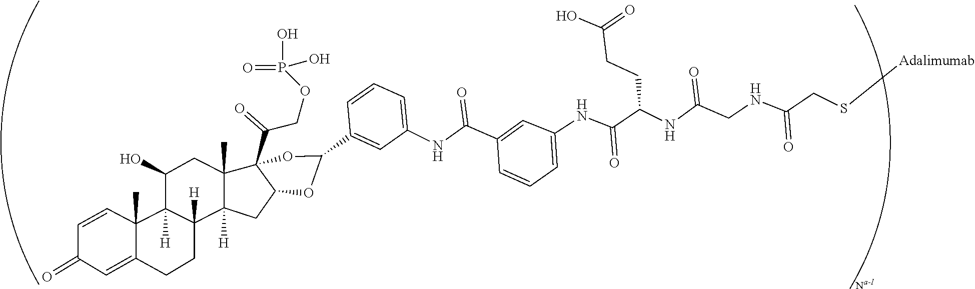 |
| I-C-49 | 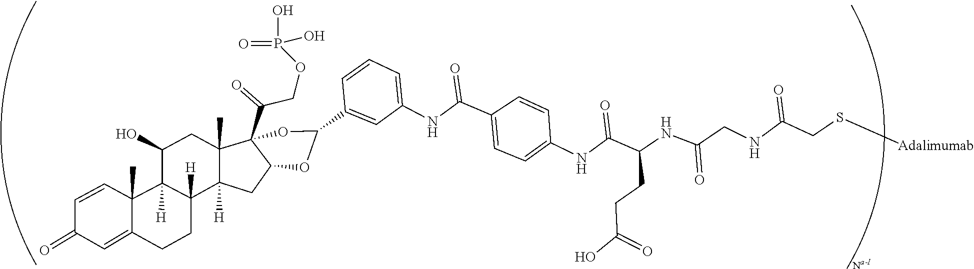 |
| I-C-51 | 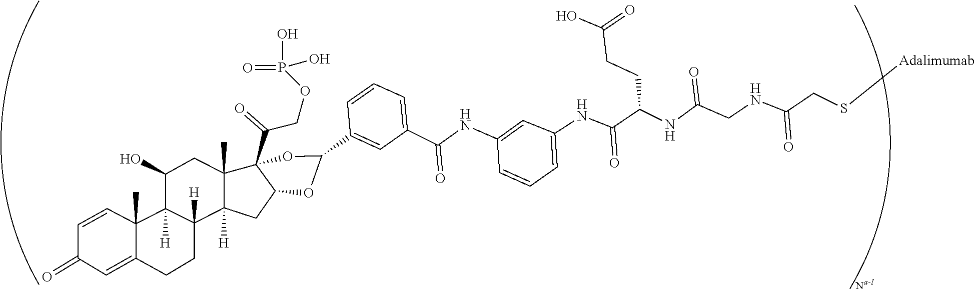 |
| I-C-53 | 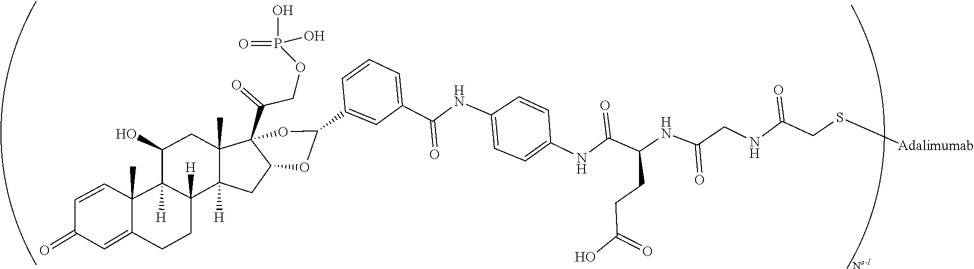 |
| I-C-55 | 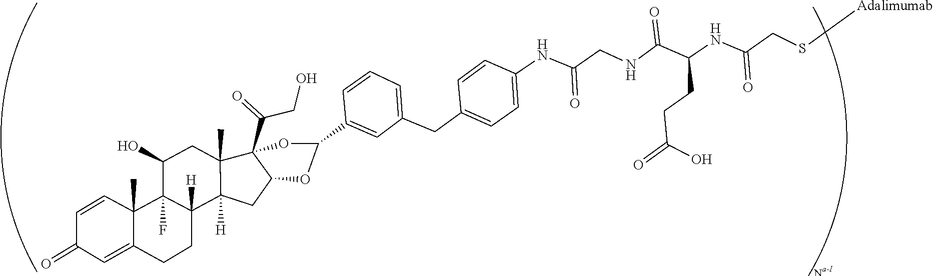 |
| I-C-56 | 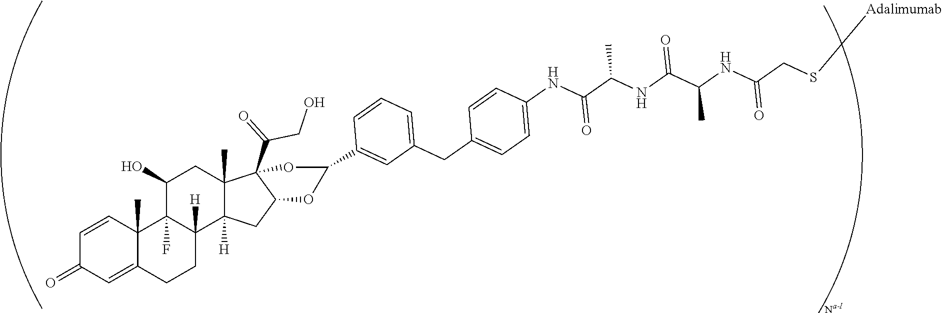 |
| I-C-57 | 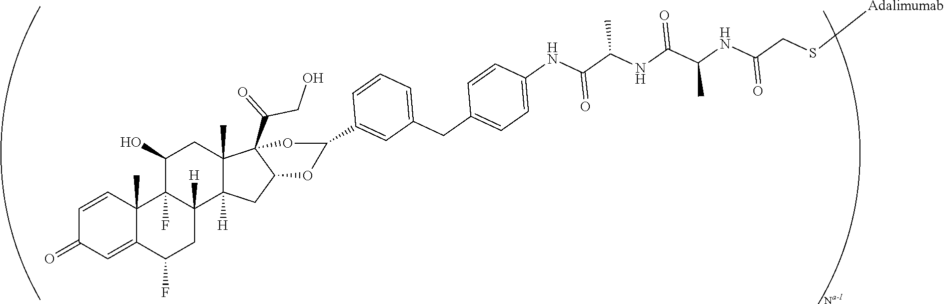 |
| I-C-58 | 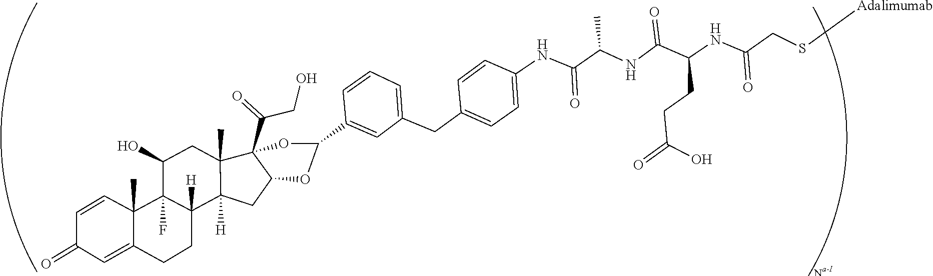 |
| I-C-59 | 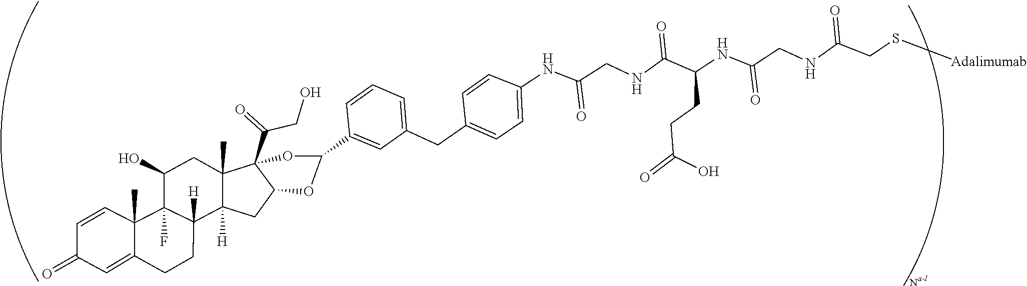 |
| I-C-60 | 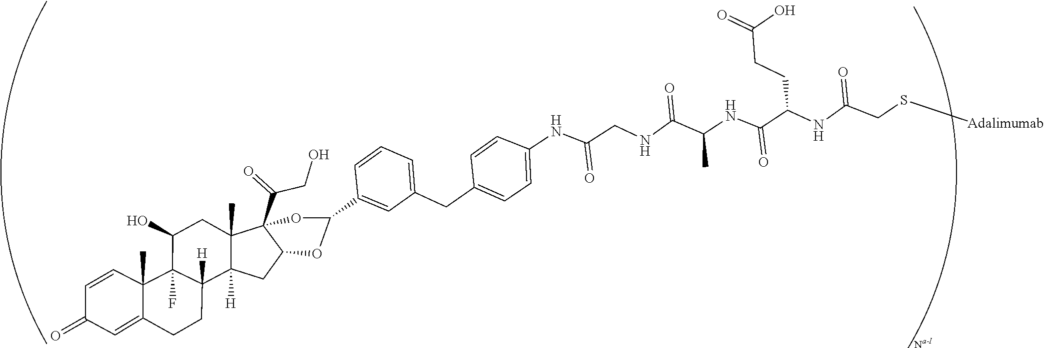 |
| I-C-61 | 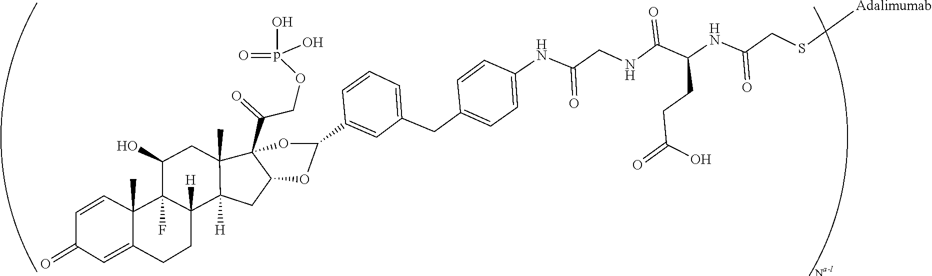 |
| I-C-62 | 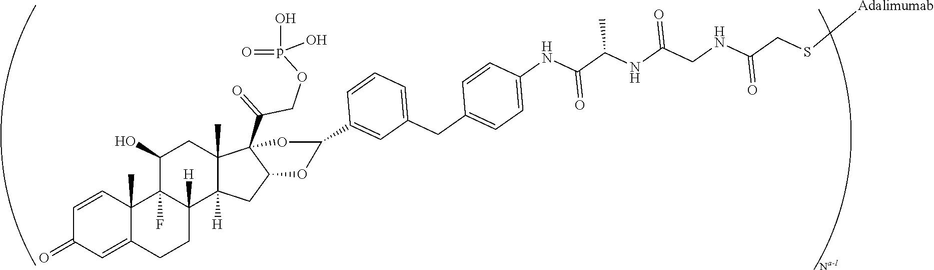 |
| I-C-63 | 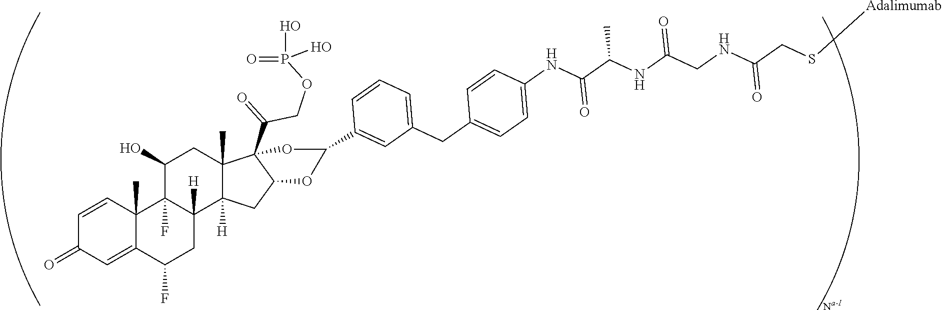 |
| I-C-64 | 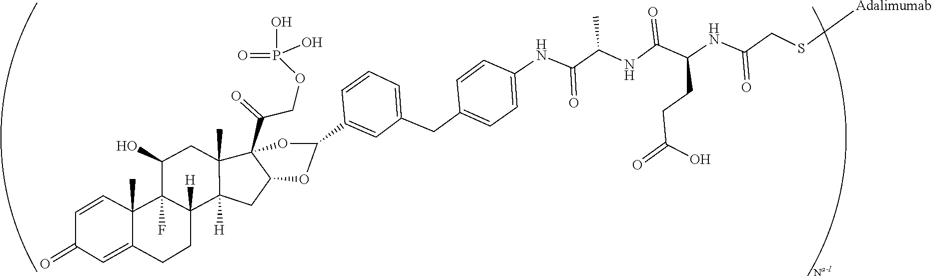 |
| I-C-65 | 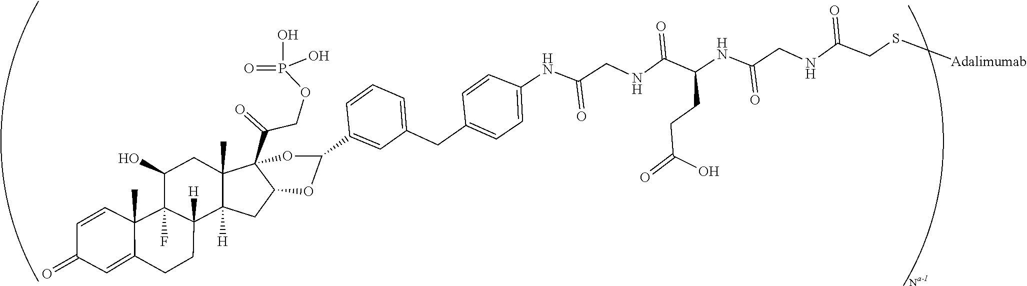 |
| I-C-66 | 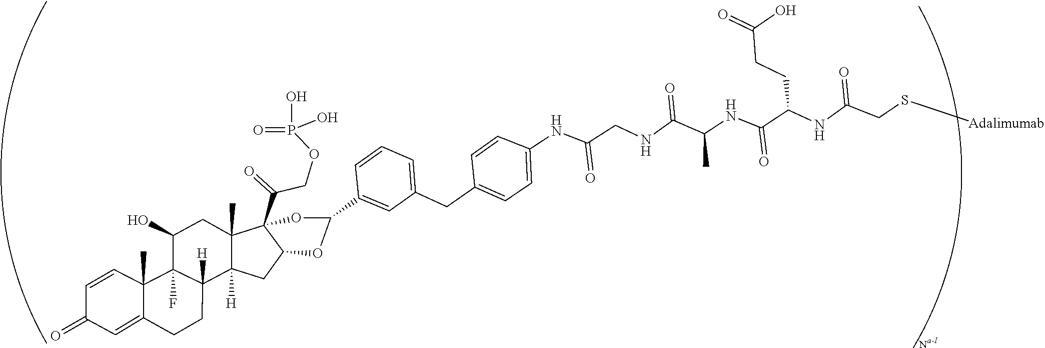 |
| I-C-67 | 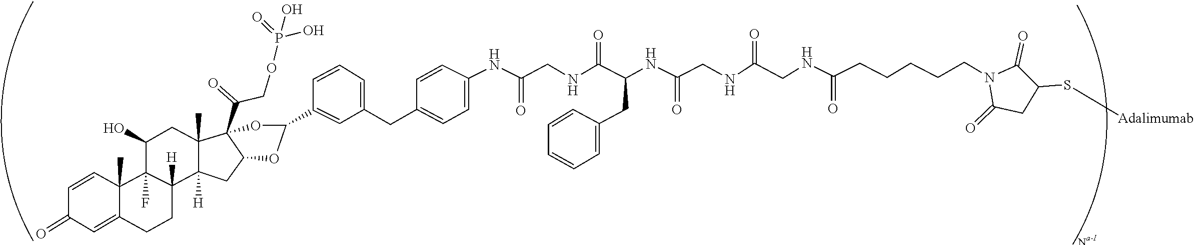 |
| I-C-68 | 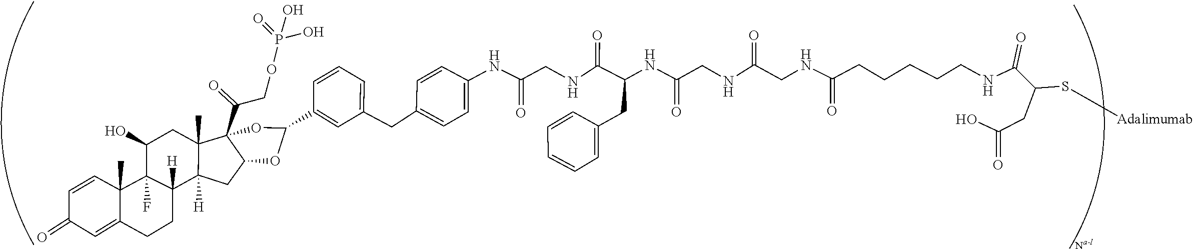 |
| I-C-69 | 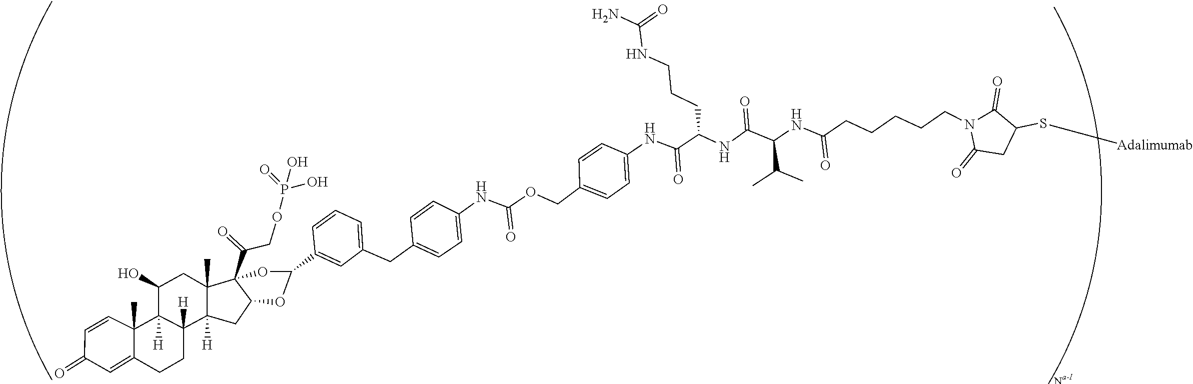 |
| I-C-70 | 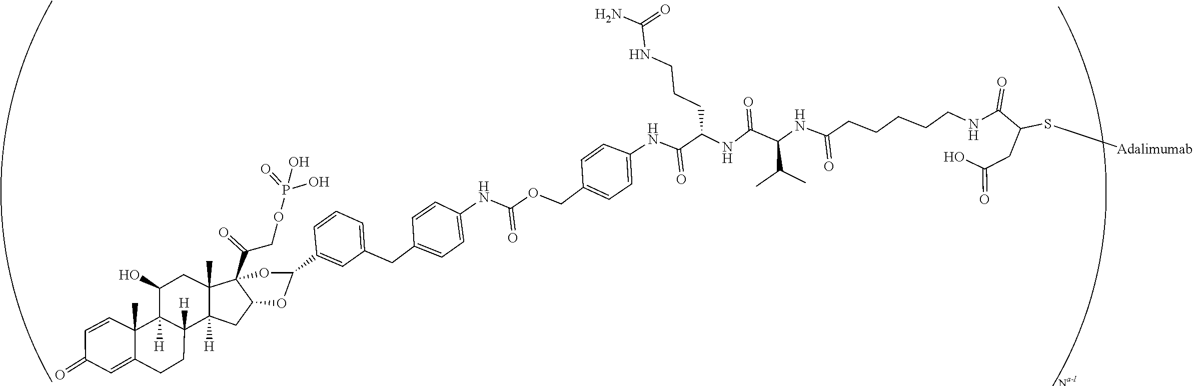 |
| I-C-71 | 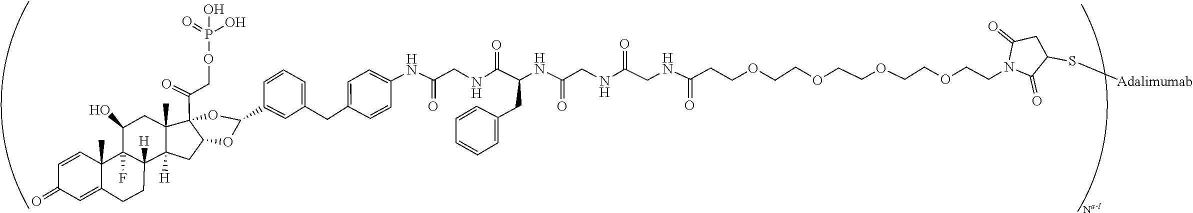 |
| I-C-72 | 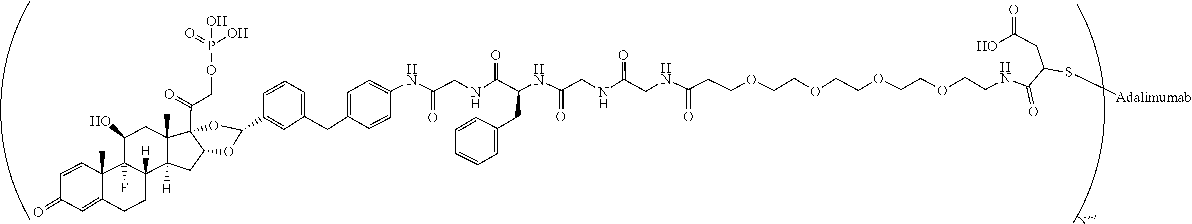 |
| I-C-73 | 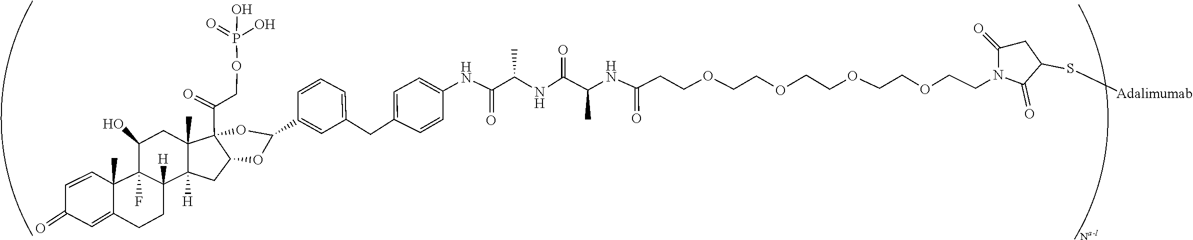 |
| I-C-74 | 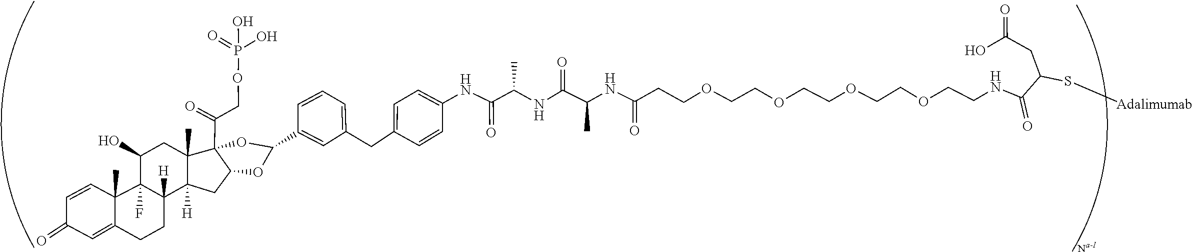 |
| I-C-75 | 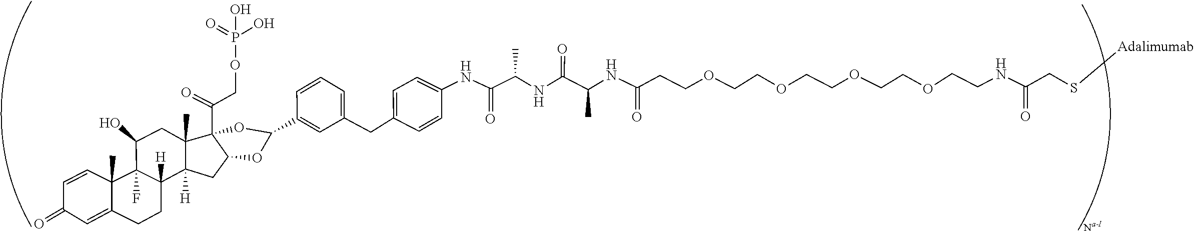 |
| I-C-76 | 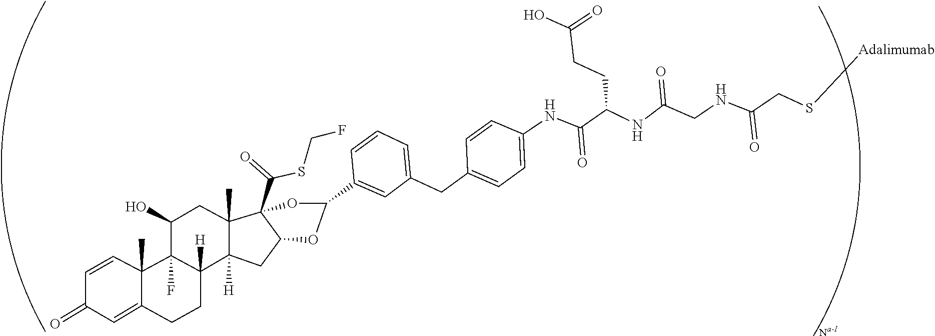 |
| I-C-77 | 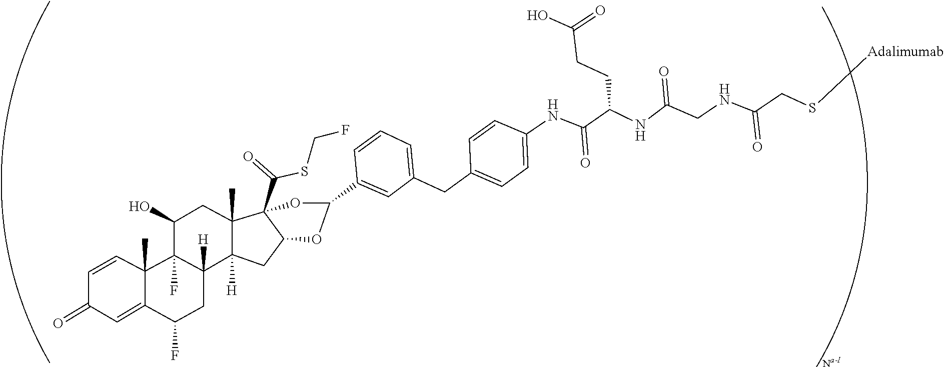 |
| I-C-78 | 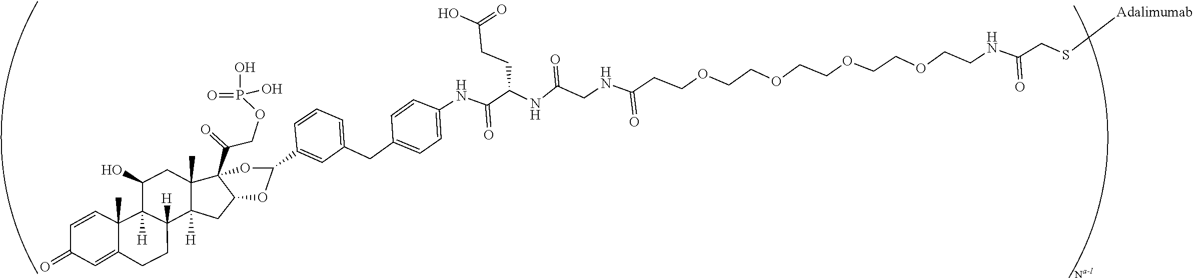 |
| I-C-79 | 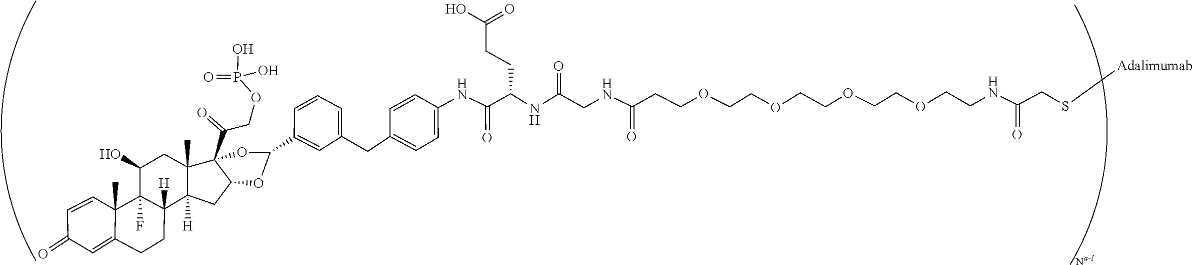 |
| I-C-80 | 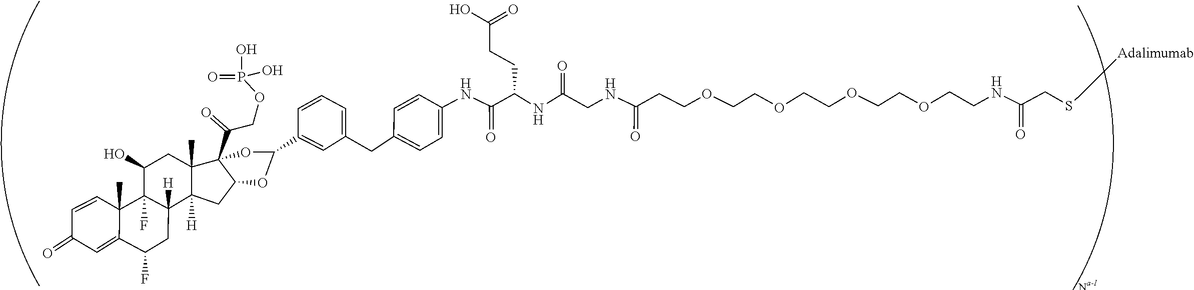 |
| I-C-81 | 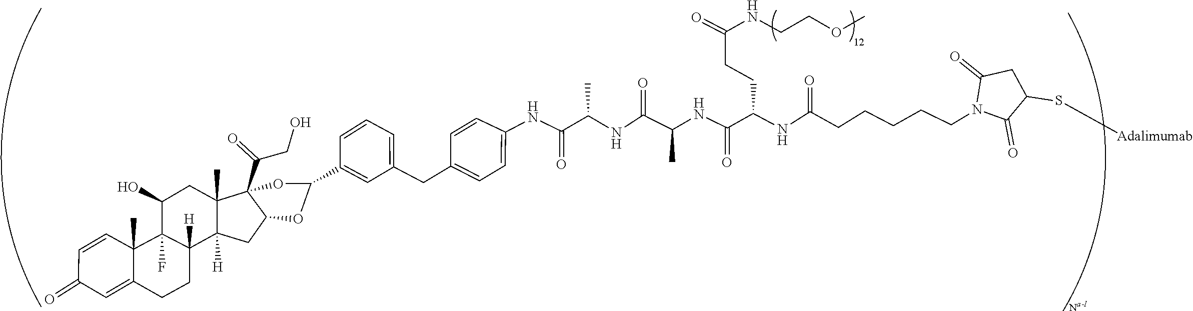 |
| I-C-82 | 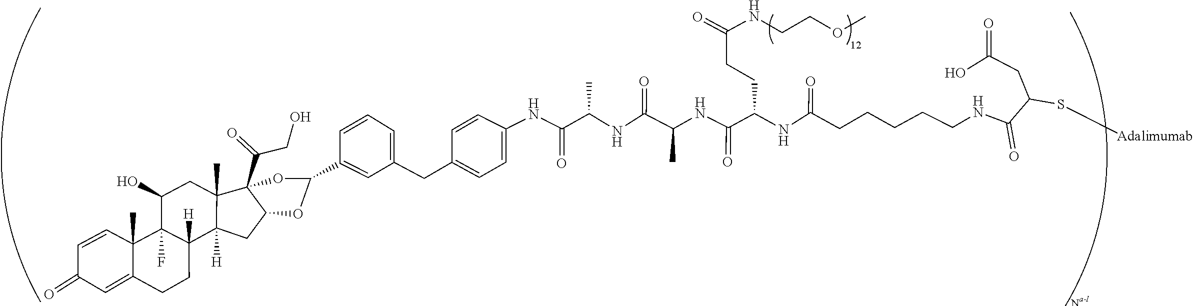 |
| I-C-83 | 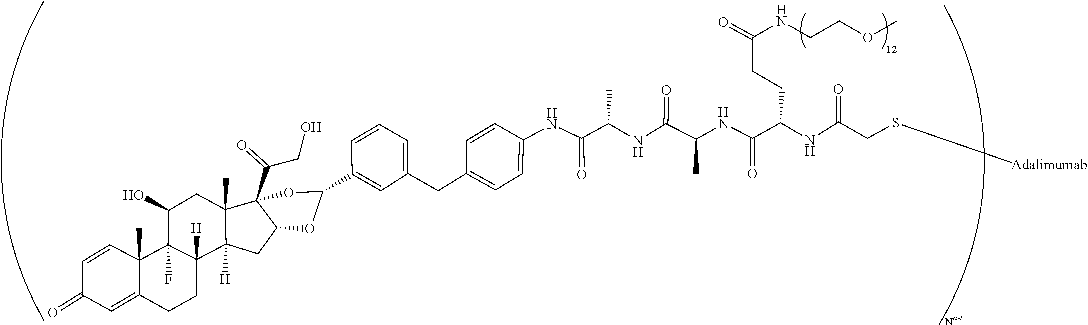 |
| I-C-84 | 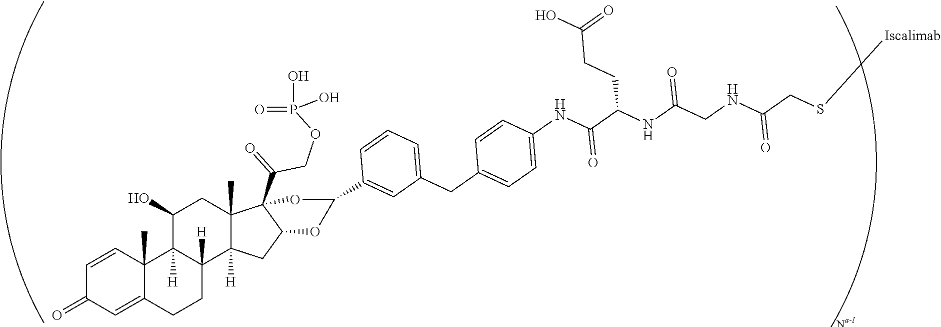 |
| I-C-85 | 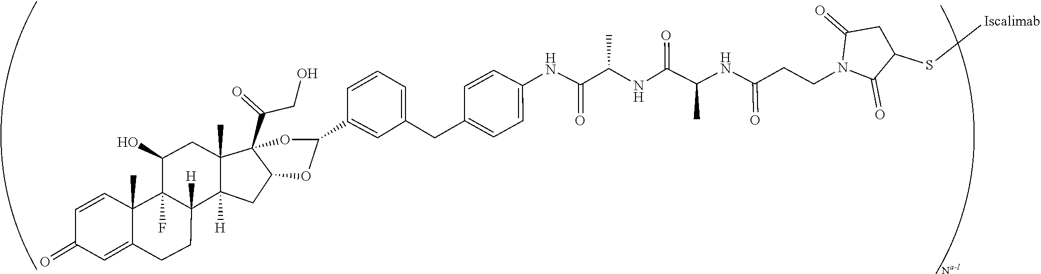 |
| I-C-88 | 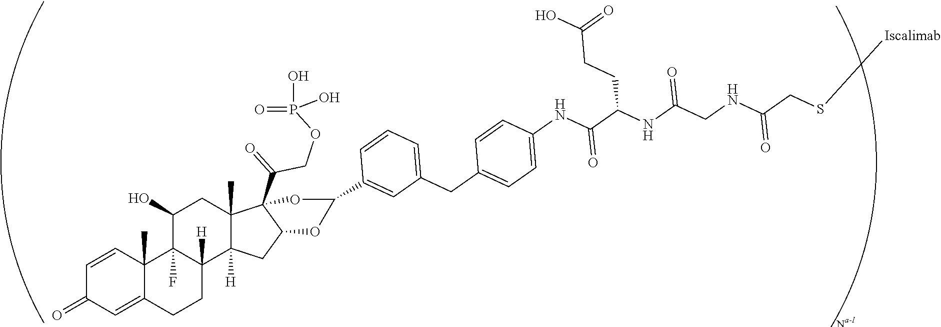 |
| I-C-94 | 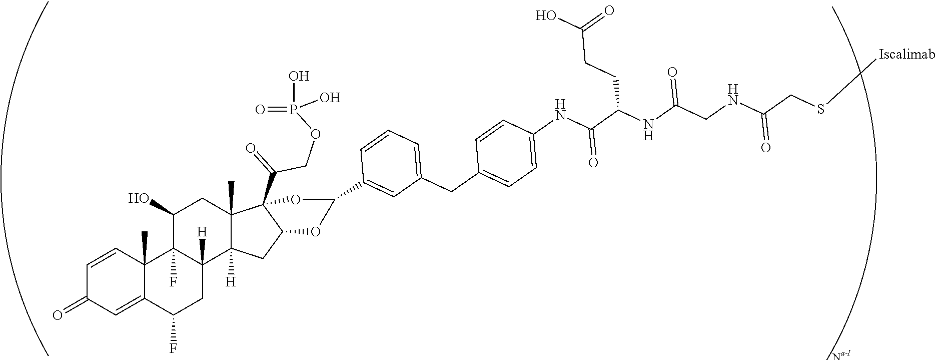 |
| I-C- 138 | 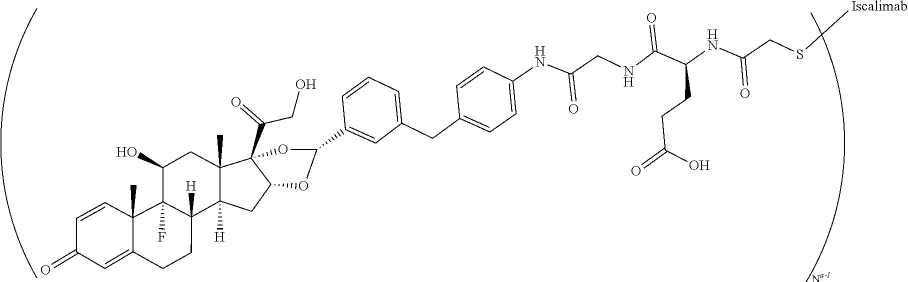 |
| I-C- 139 | 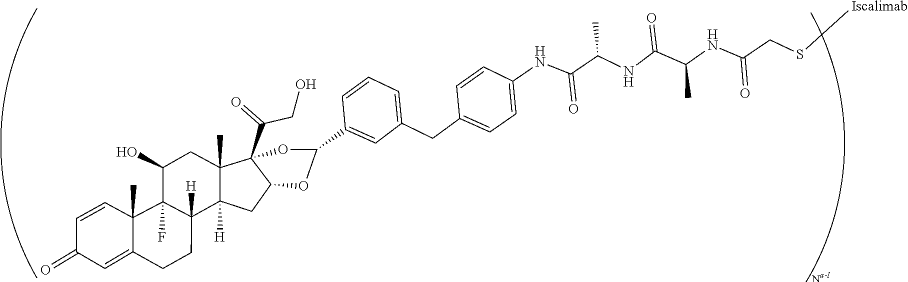 |
| I-C- 140 | 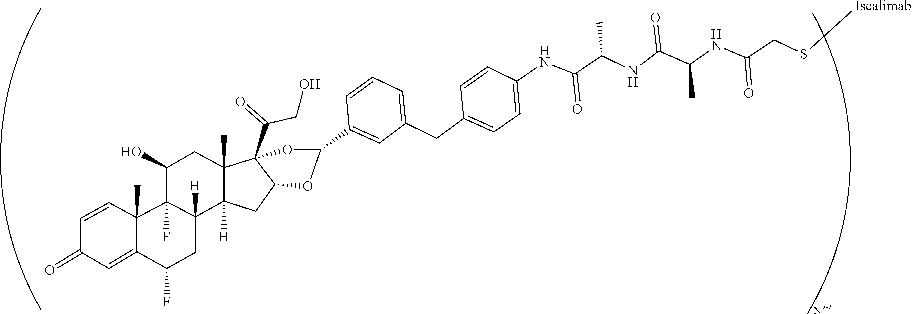 |
| I-C- 145 | 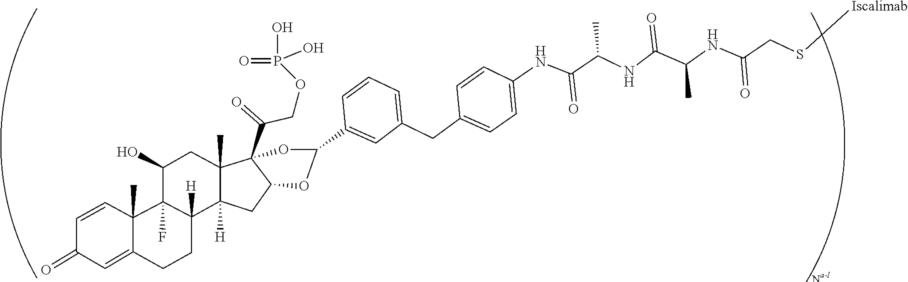 |
| I-C- 146 | 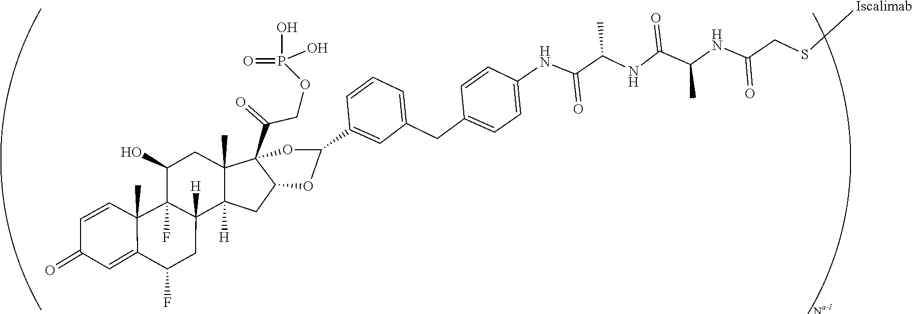 |
| I-C- 147 | 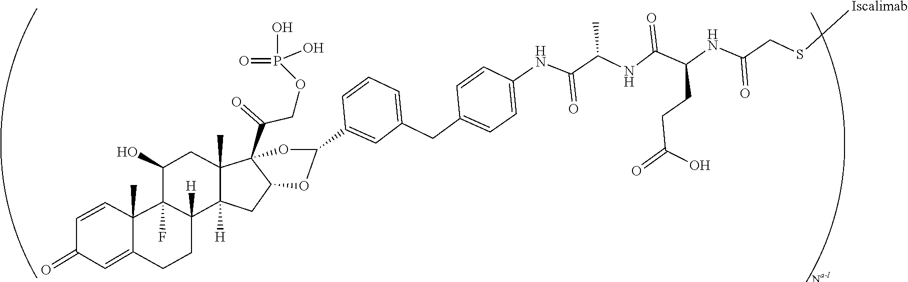 |
| I-C- 148 | 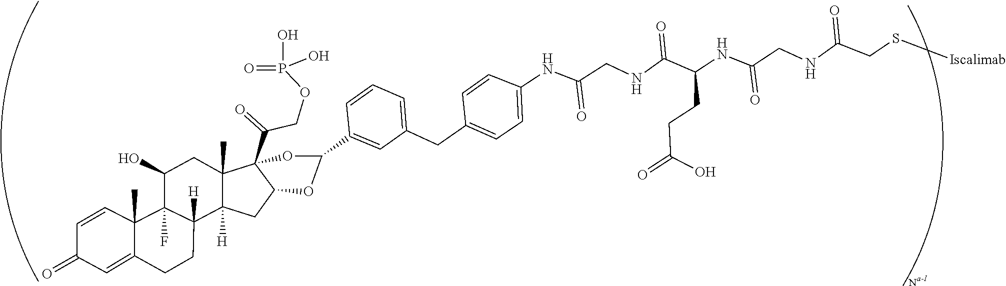 |
| I-C- 149 | 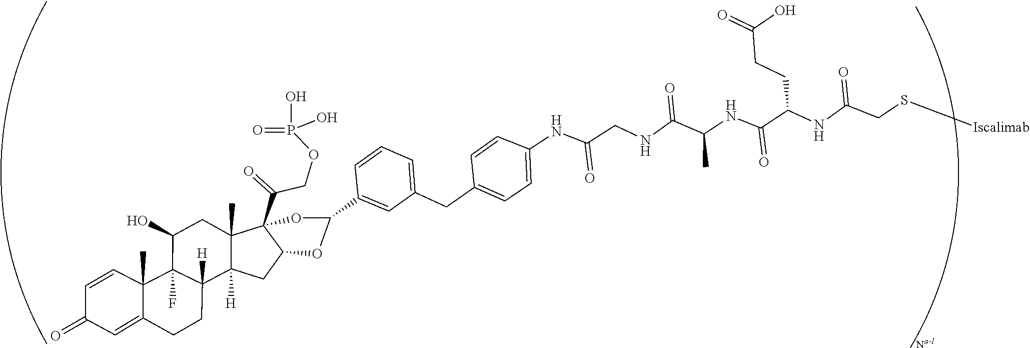 |
| I-C- 159 | 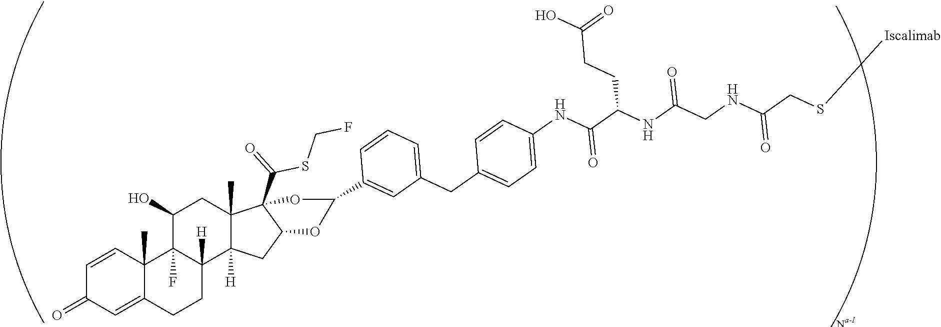 |
| I-C- 160 | 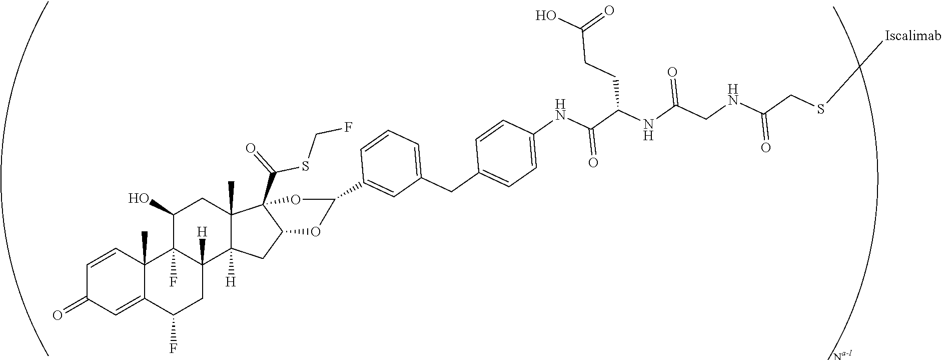 |
| I-C- 167 | 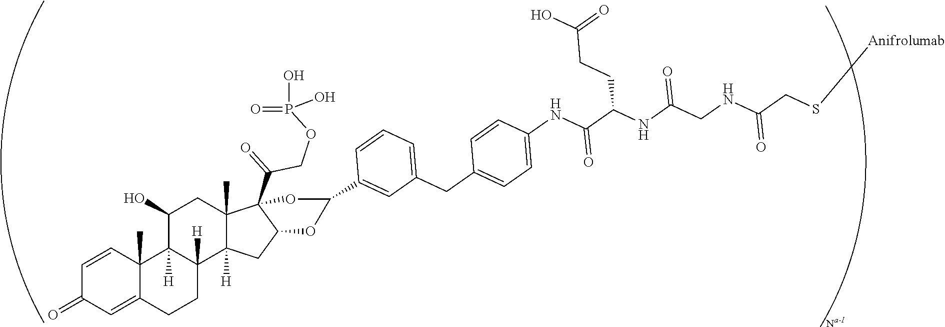 |
| I-C- 168 | 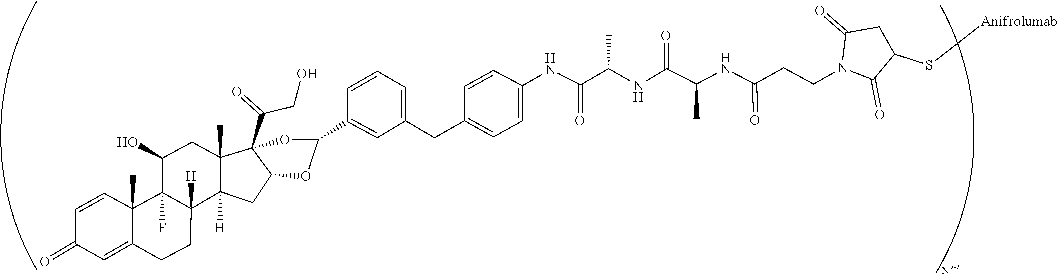 |
| I-C- 171 | 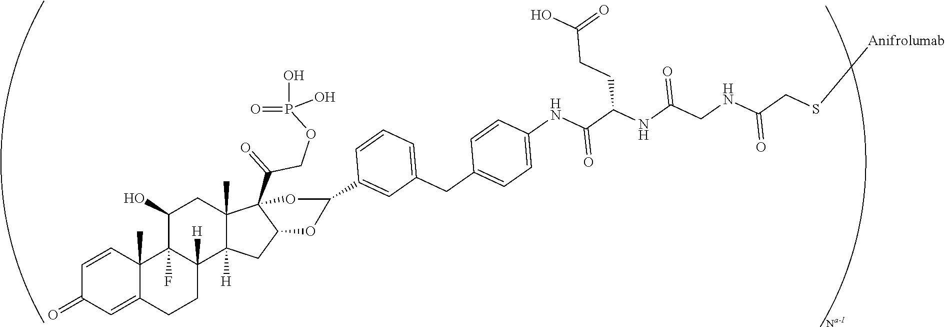 |
| I-C- 177 | 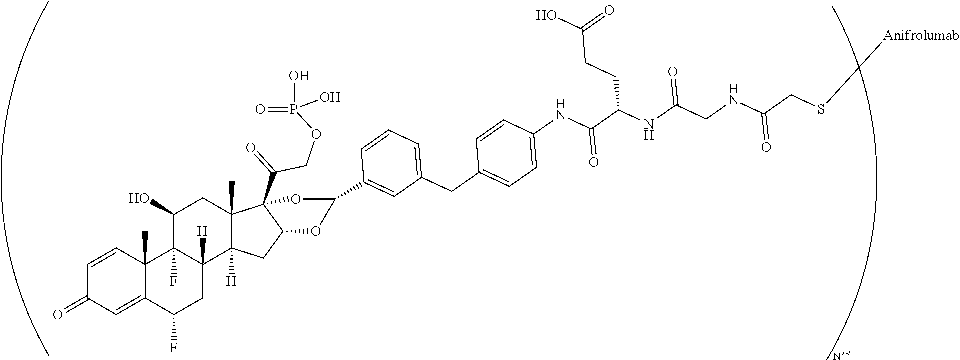 |

The present application provides a compound or a tautomer, a mesomer, a racemate, an enantiomer or a diastereoisomer thereof, or a mixture thereof, or a pharmaceutically acceptable salt thereof, wherein the compound comprises a structure that may be selected from the group

| No. | Structure |
|---|---|
| II-C-1 | 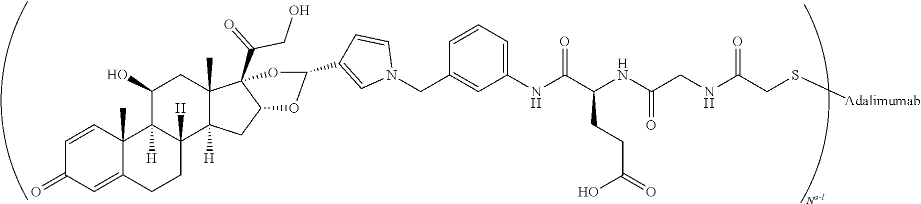 |
| II-C-2 | 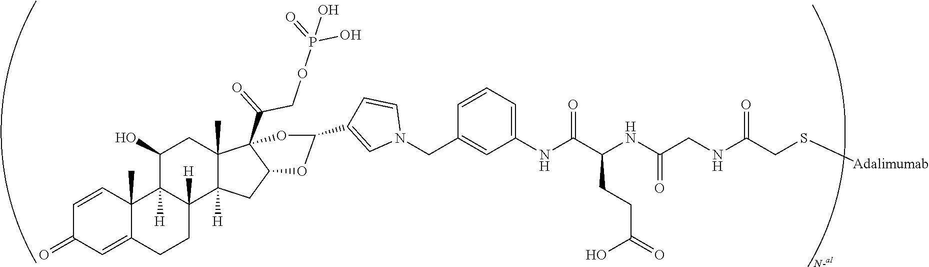 |
| II-C-7 | 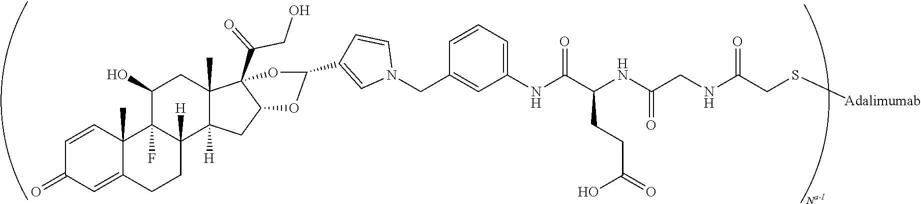 |
| II-C-8 | 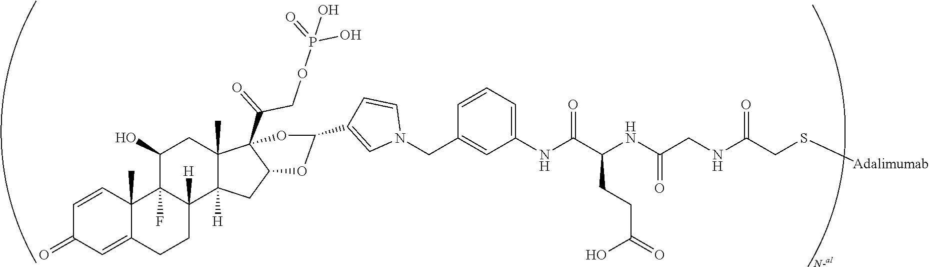 |
| II-C-12 | 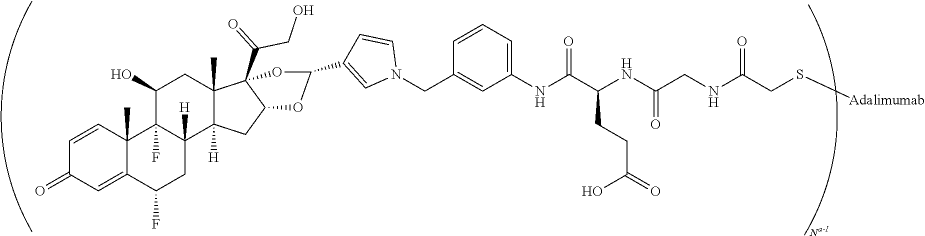 |
| II-C-13 | 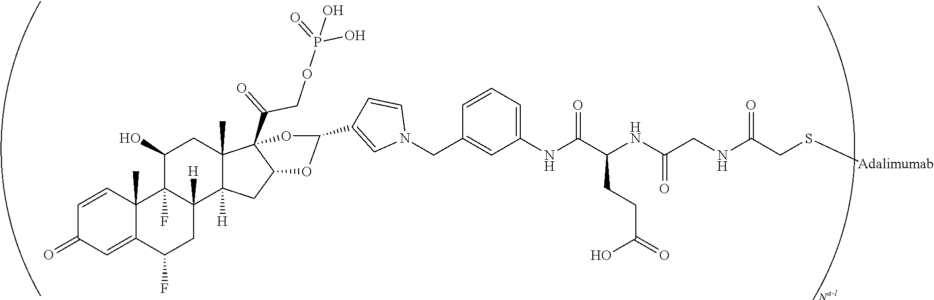 |
| II-C-17 | 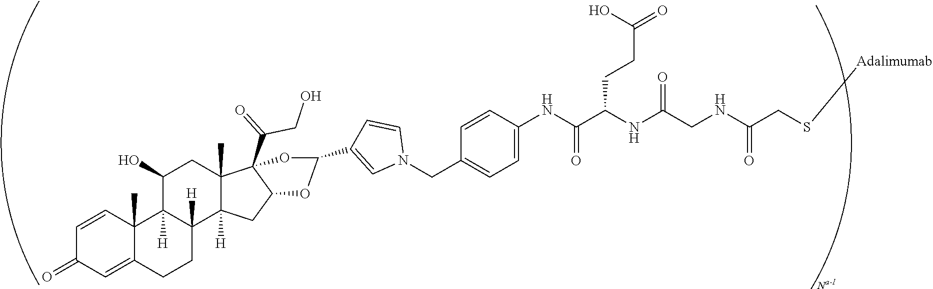 |
| II-C-18 | 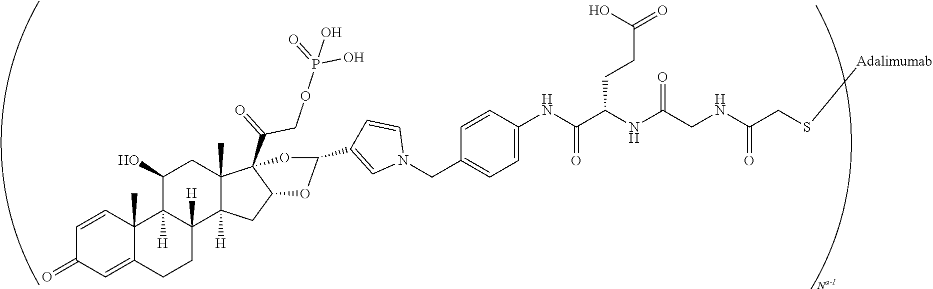 |
| II-C-23 | 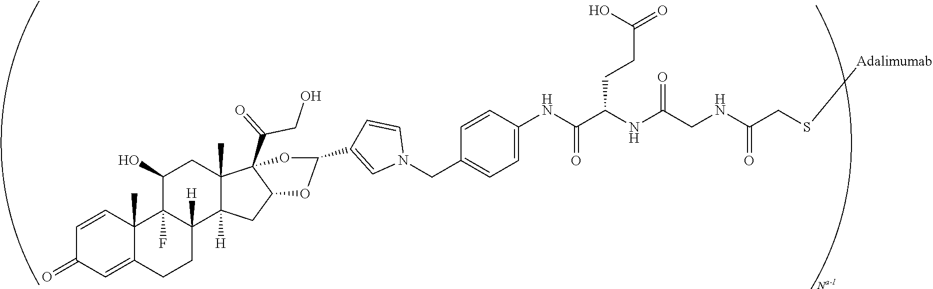 |
| II-C-24 | 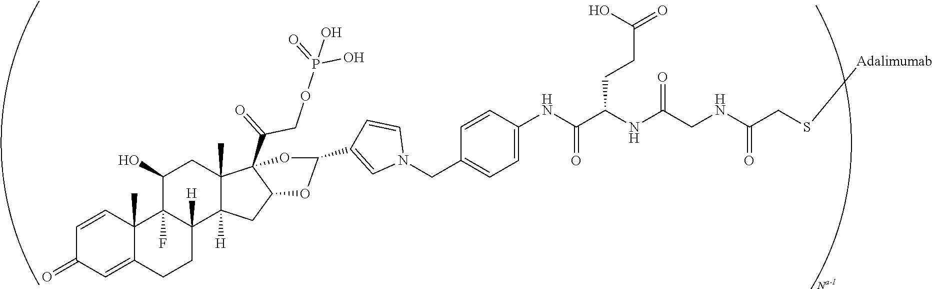 |
| II-C-29 | 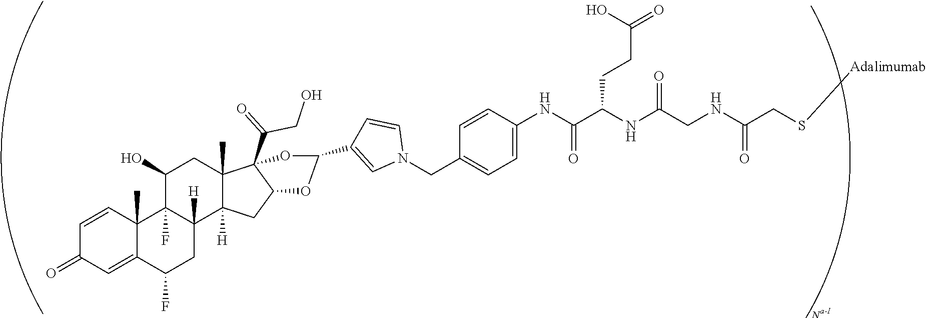 |
| II-C-30 | 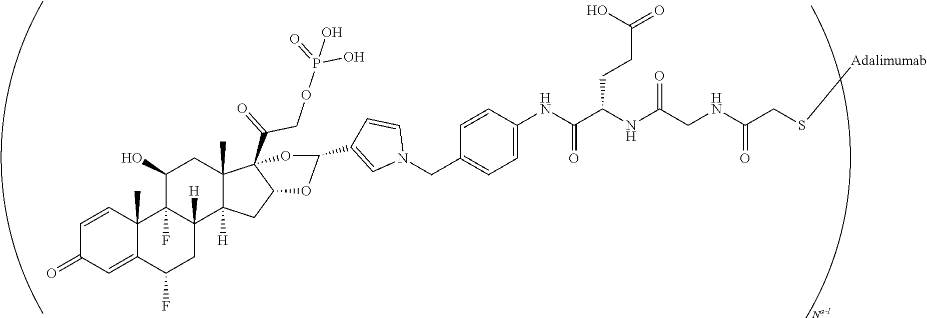 |
| II-C-34 | 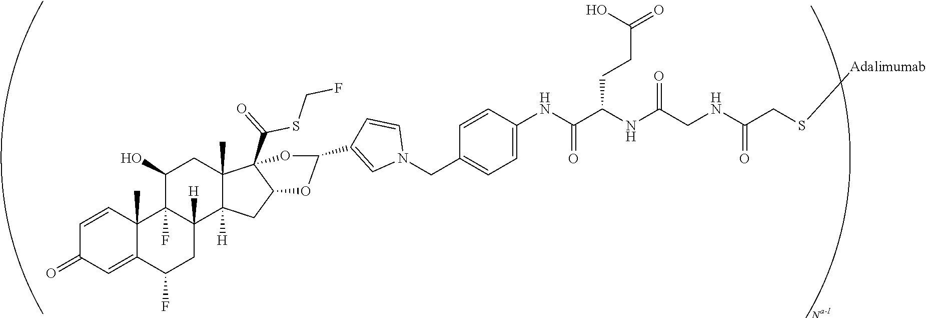 |
| II-C-35 | 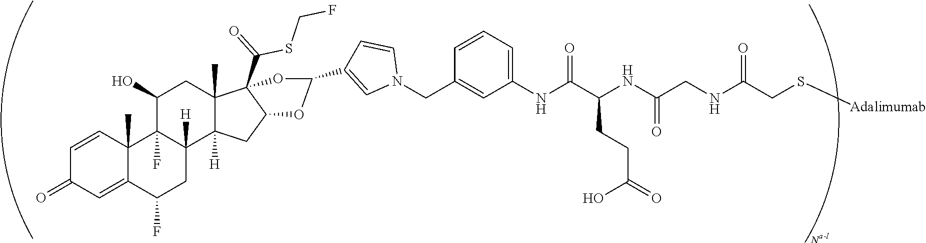 |
| II-C-36 | 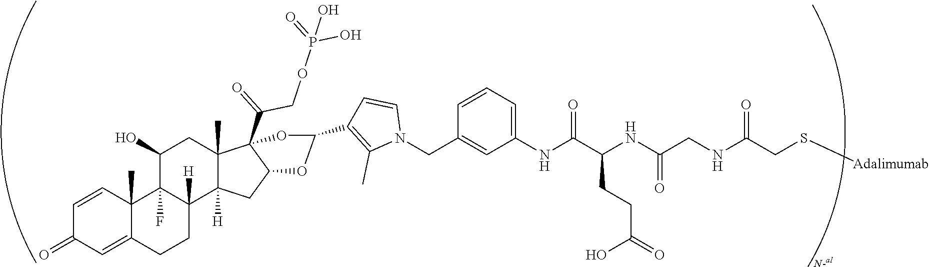 |
| II-C-39 | 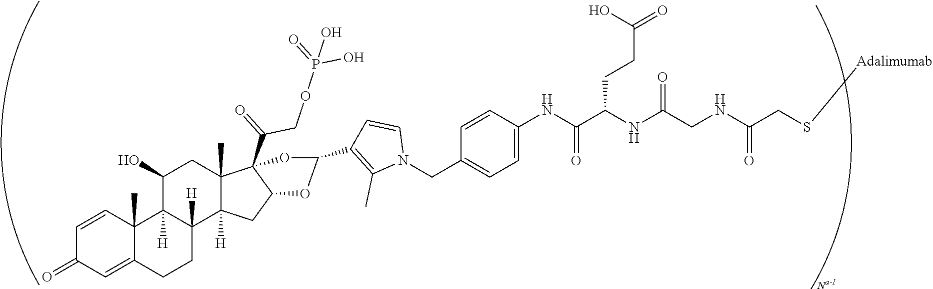 |
| II-C-42 | 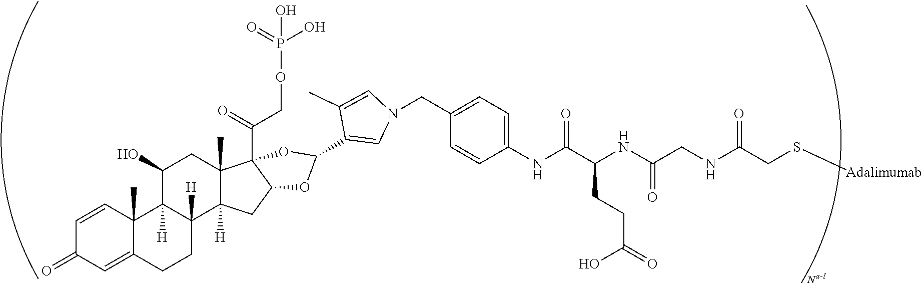 |
| II-C-45 | 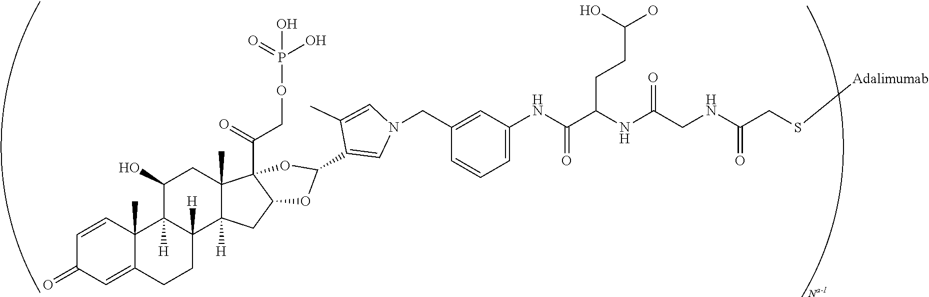 |
| II-C-48 | 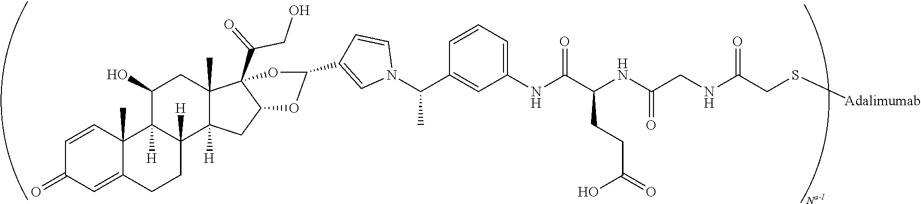 |
| II-C-49 | 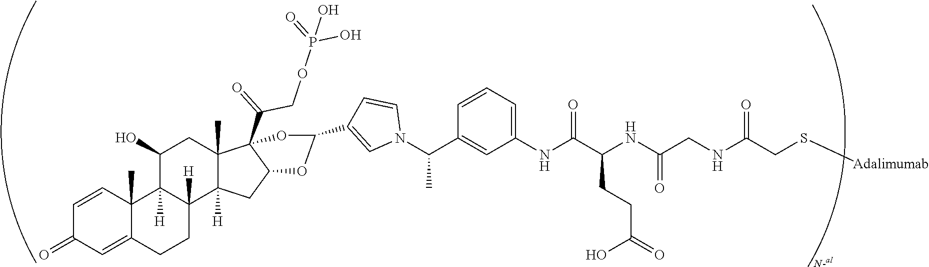 |
| II-C-53 | 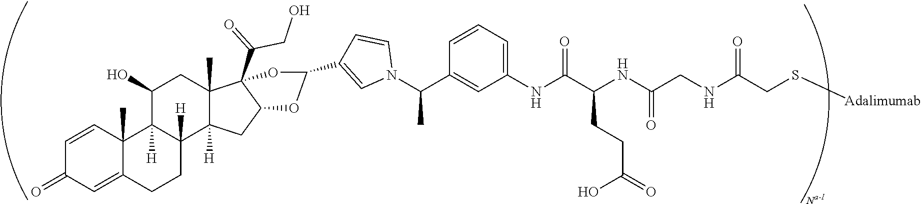 |
| II-C-54 | 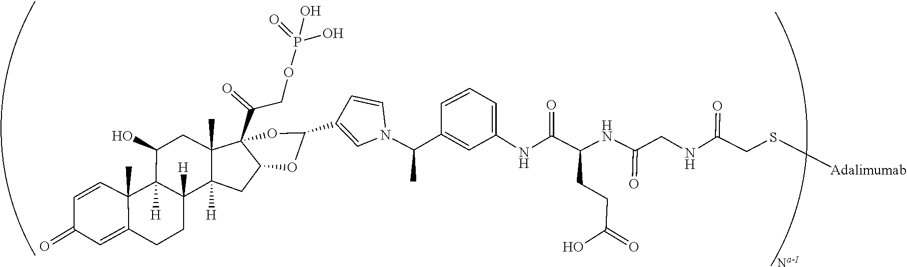 |
| II-C-78 | 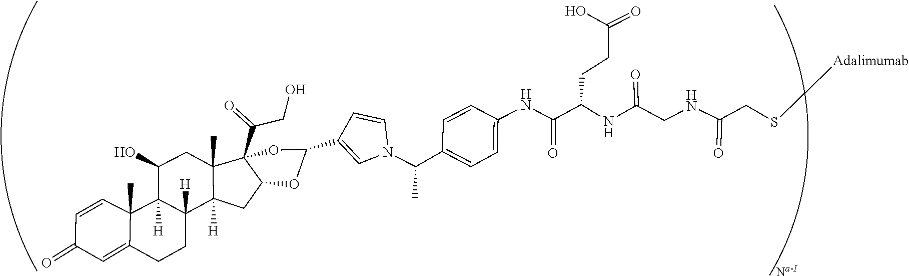 |
| II-C-79 | 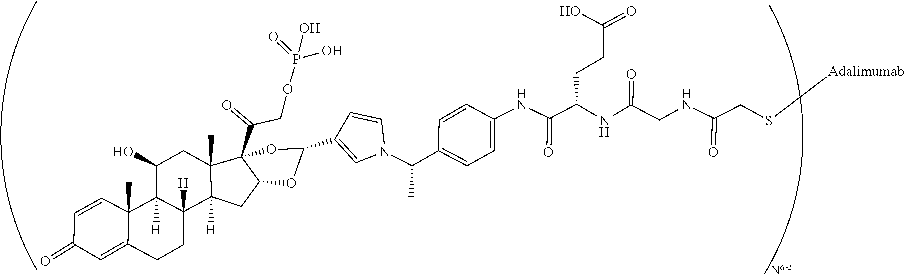 |
| II-C-92 | 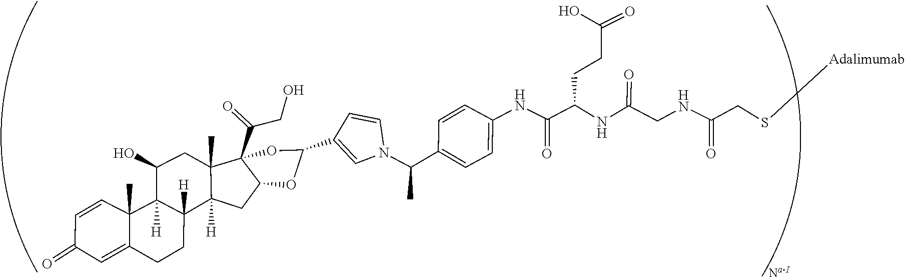 |
| II-C-93 | 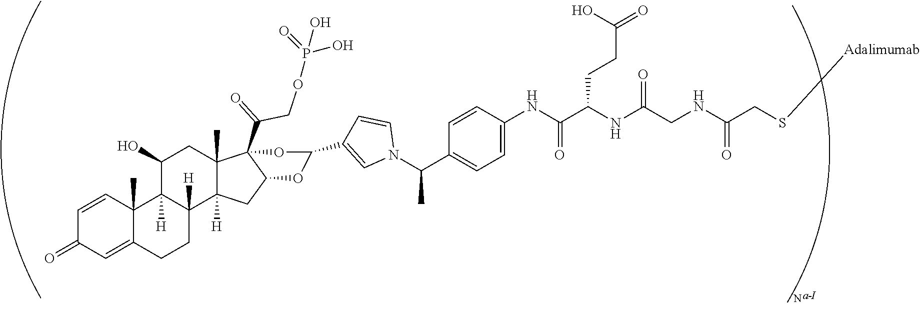 |
| II-C-107 | 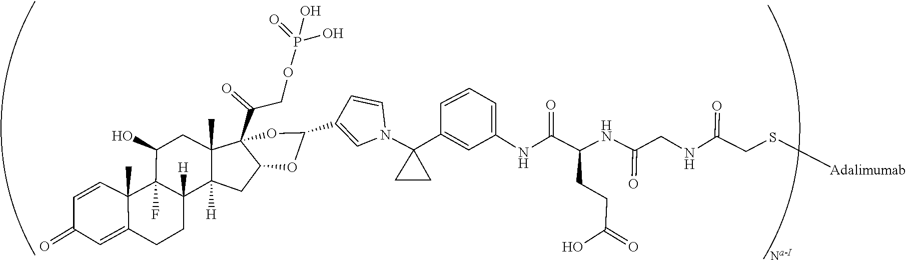 |
| II-C-108 | 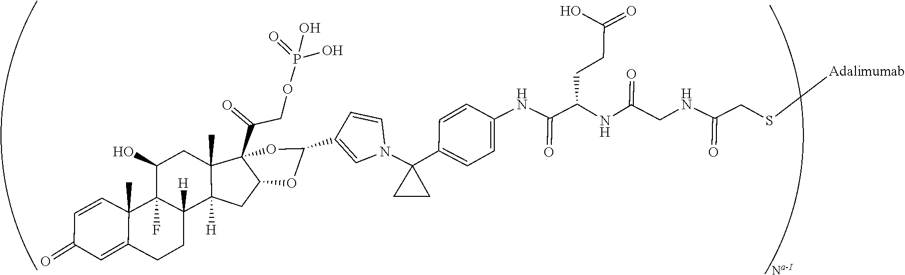 |
| II-C-111 | 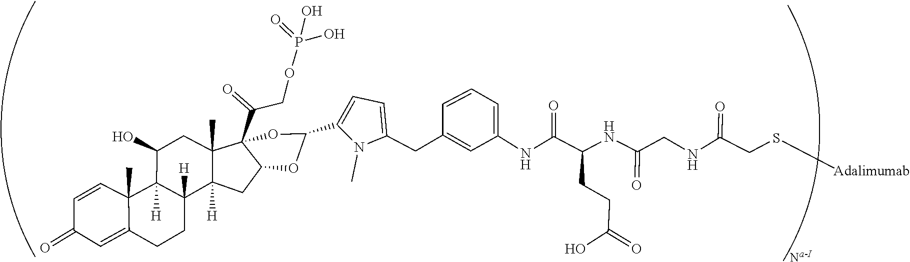 |
| II-C-114 | 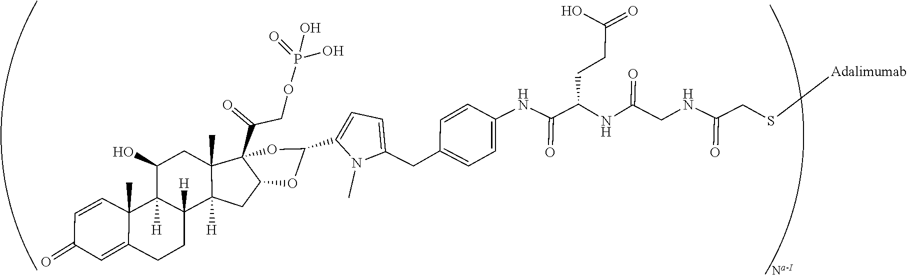 |
| II-C-117 | 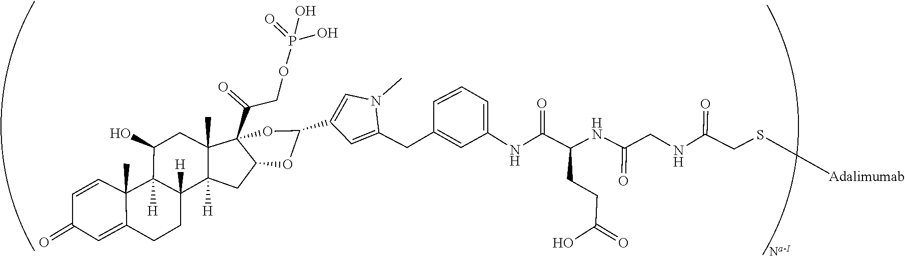 |
| II-C-120 | 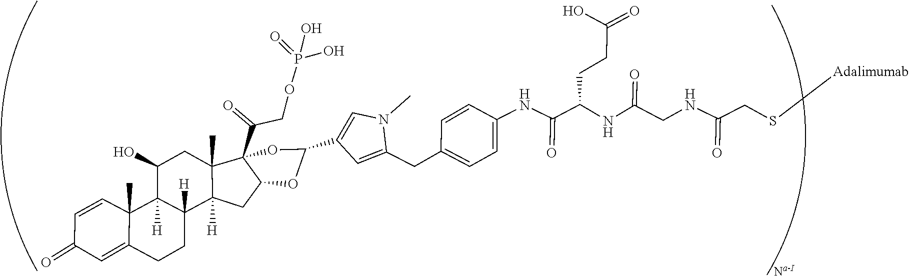 |
| II-C-123 | 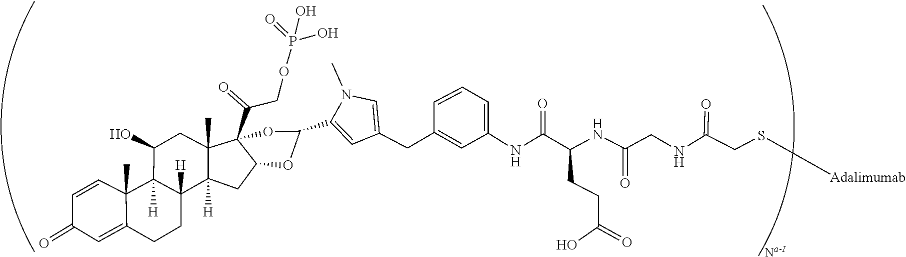 |
| II-C-126 | 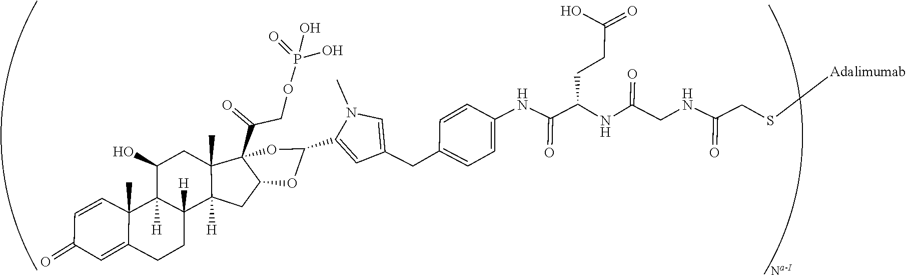 |
| II-C-165 | 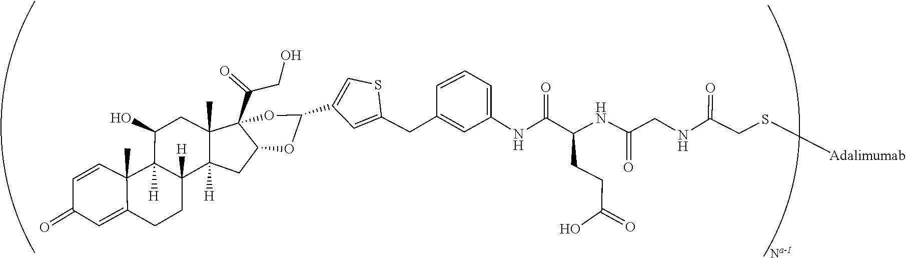 |
| II-C-166 | 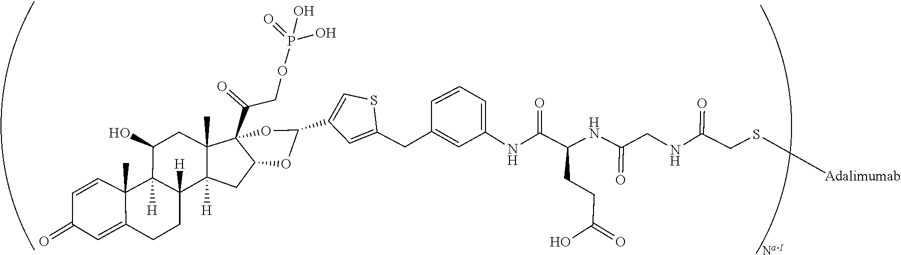 |
| II-C-170 | 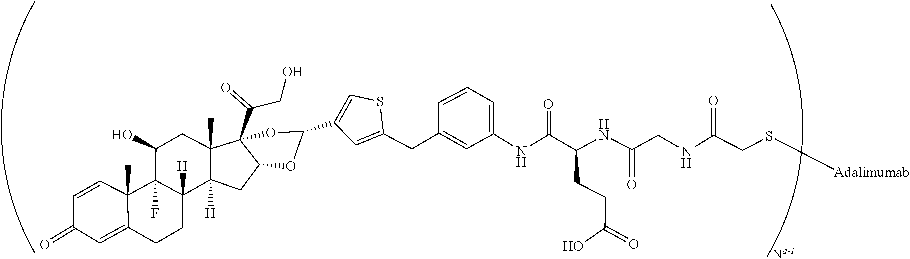 |
| II-C-171 | 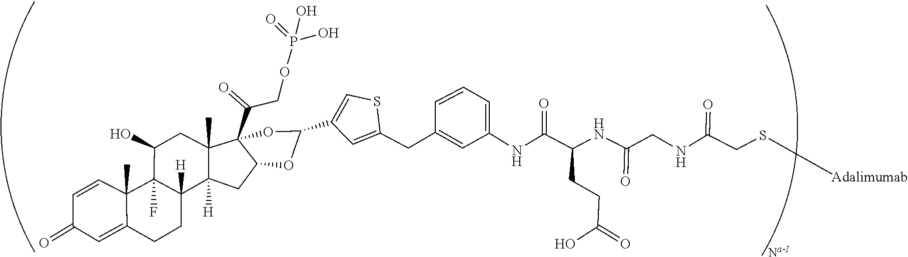 |
| II-C-175 | 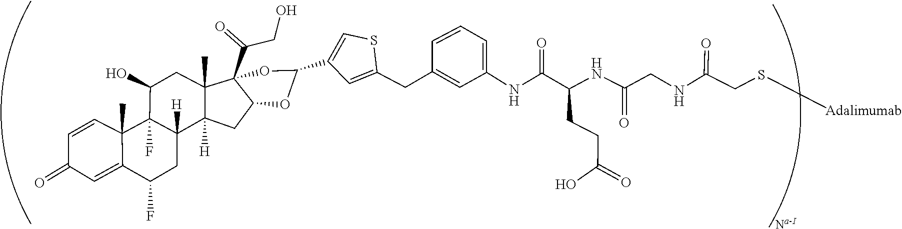 |
| II-C-176 | 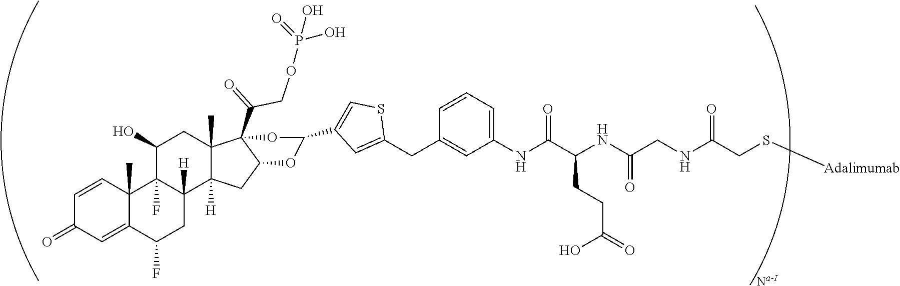 |
| II-C-180 | 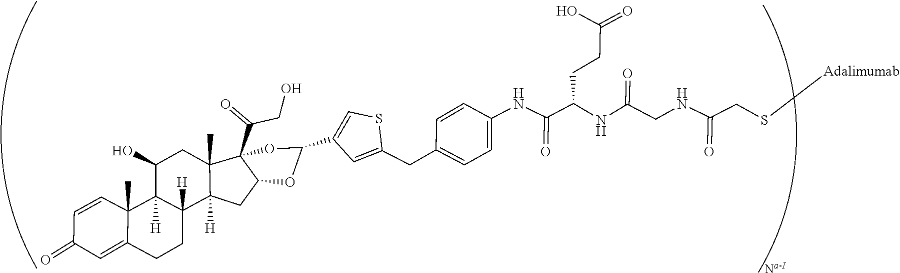 |
| II-C-181 | 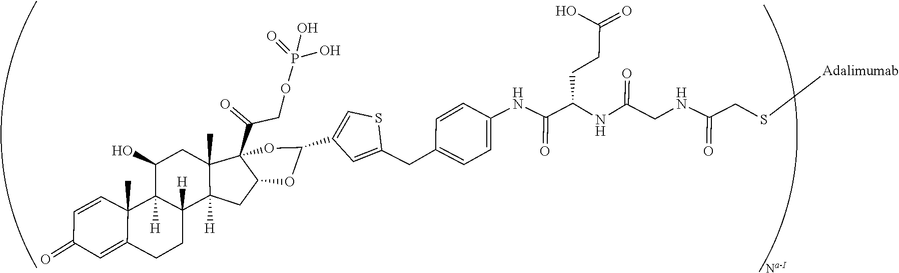 |
| II-C-185 | 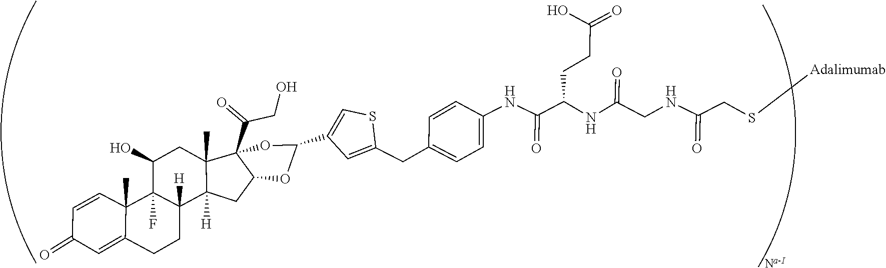 |
| II-C-186 | 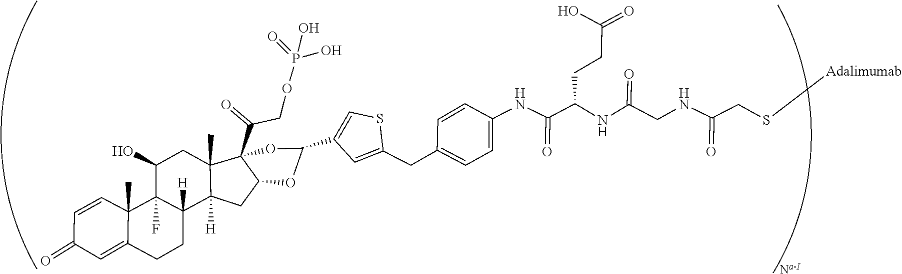 |
| II-C-190 | 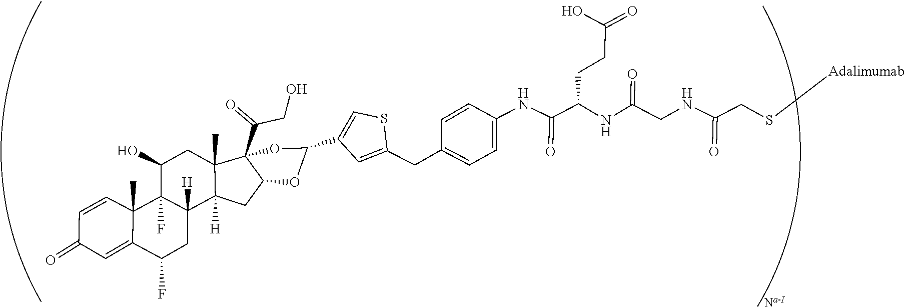 |
| II-C-191 | 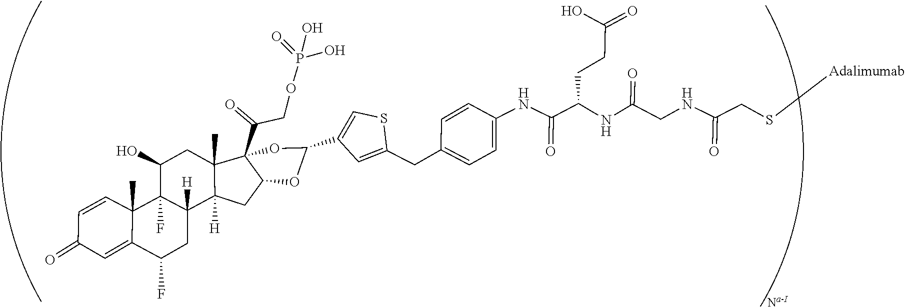 |
| II-C-195 | 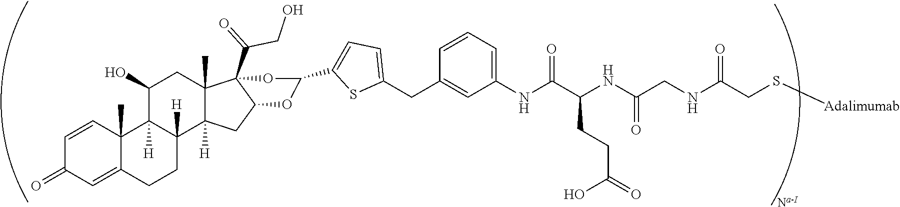 |
| II-C-196 | 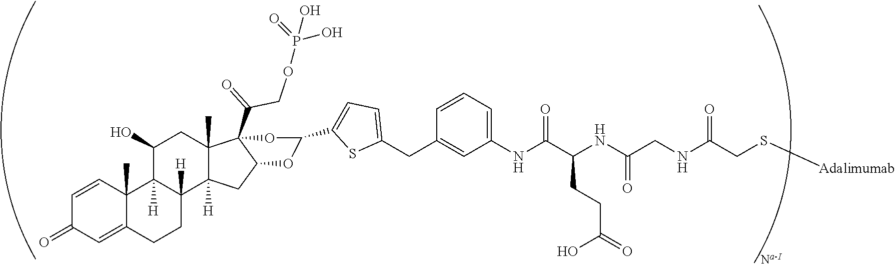 |
| II-C-200 | 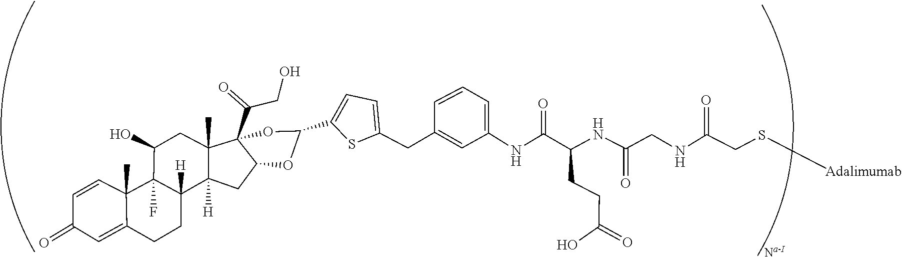 |
| II-C-201 | 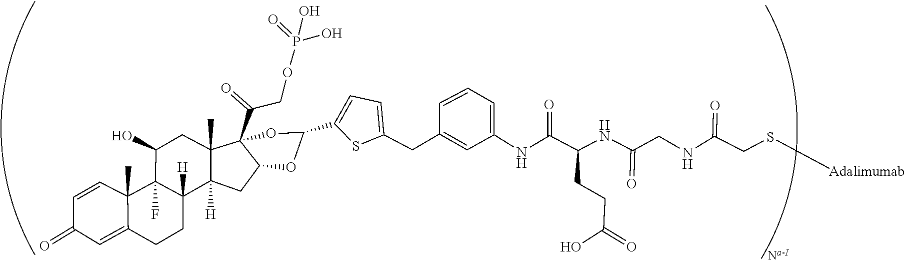 |
| II-C-205 | 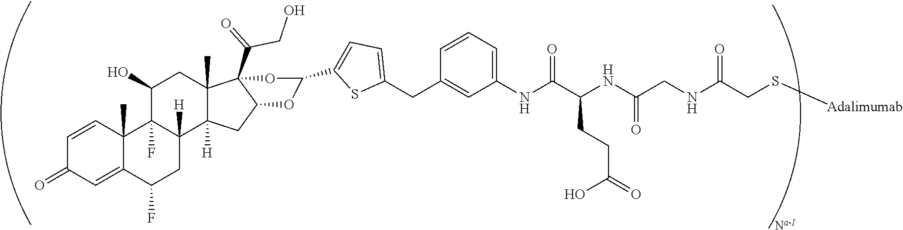 |
| II-C-206 | 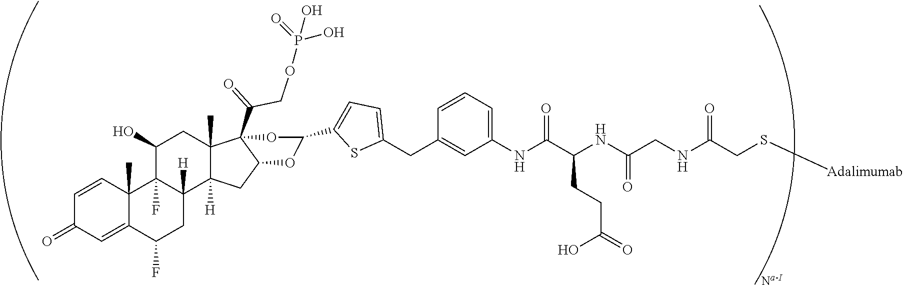 |
| II-C-210 | 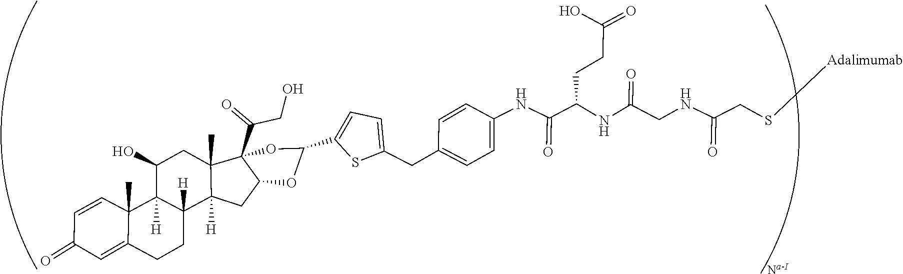 |
| II-C-211 | 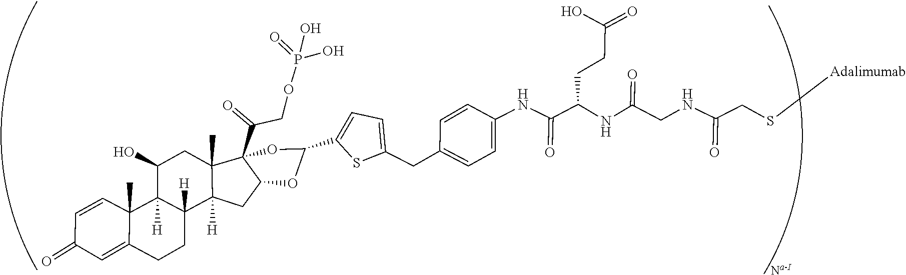 |
| II-C-215 | 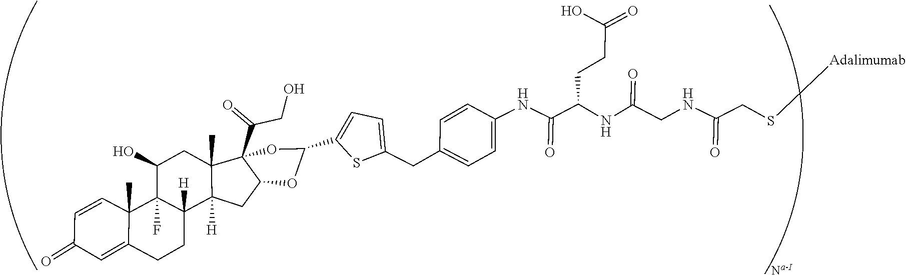 |
| II-C-216 | 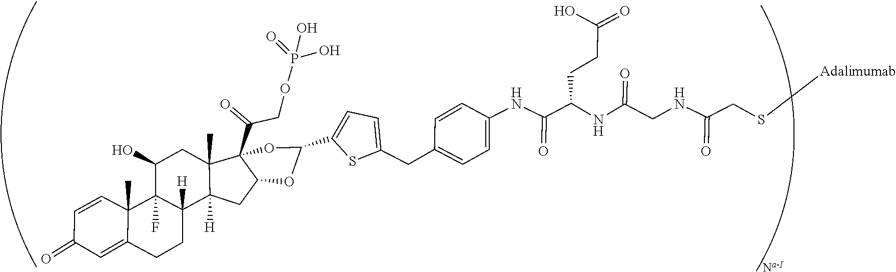 |
| II-C-220 | 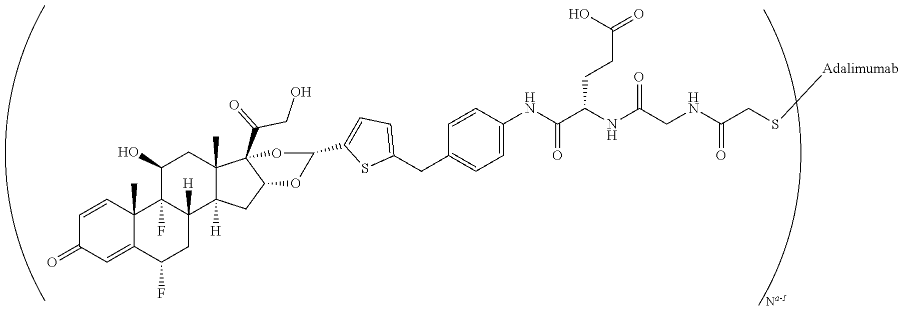 |
| II-C-221 | 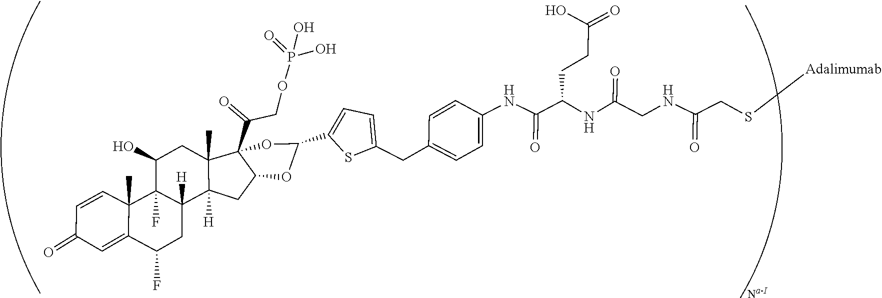 |
| II-C-233 | 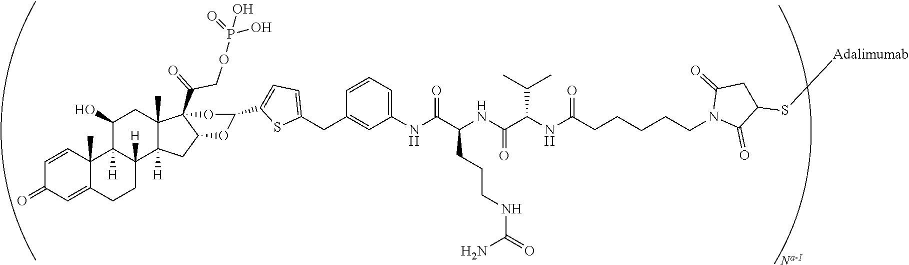 |
| II-C-234 | 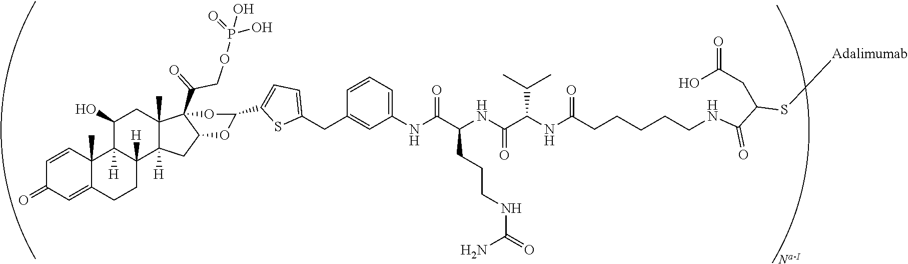 |
| II-C-235 | 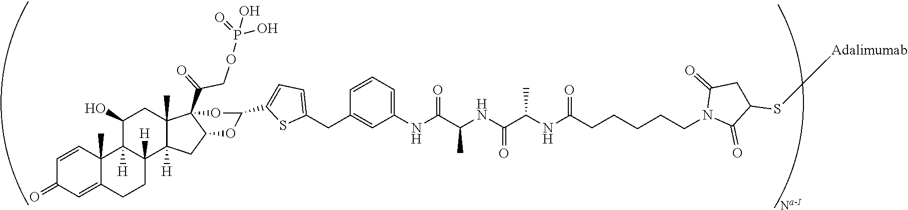 |
| II-C-236 | 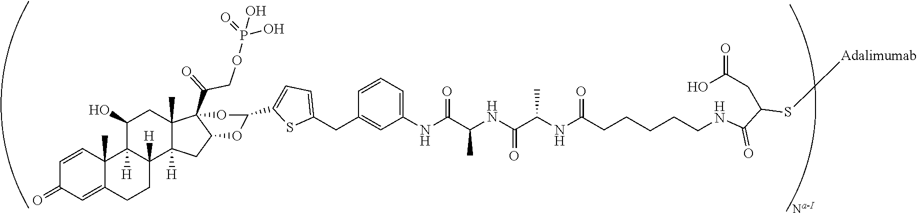 |
| II-C-237 | 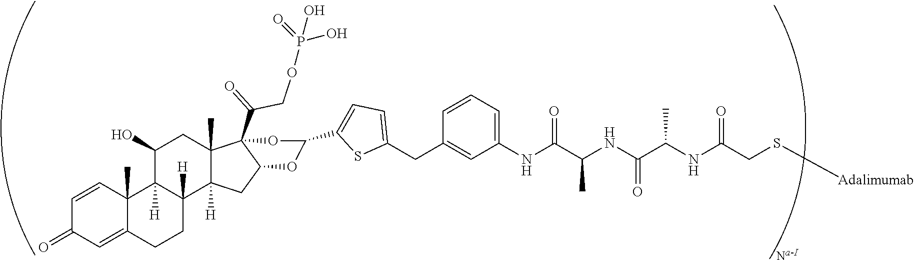 |
| II-C-238 | 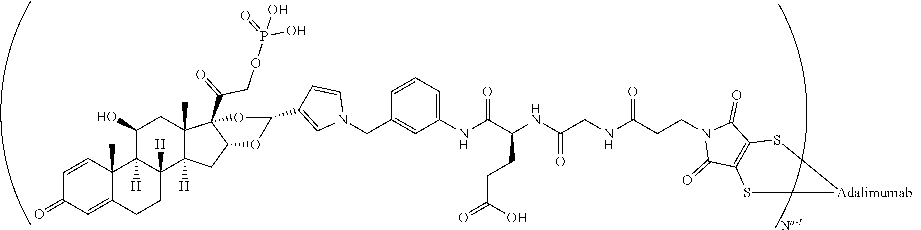 |
| II-C-239 | 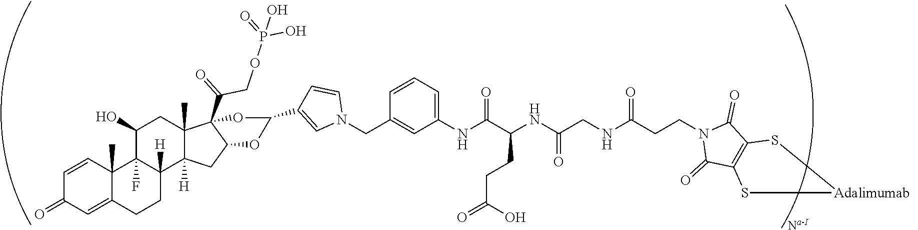 |
| II-C-240 | 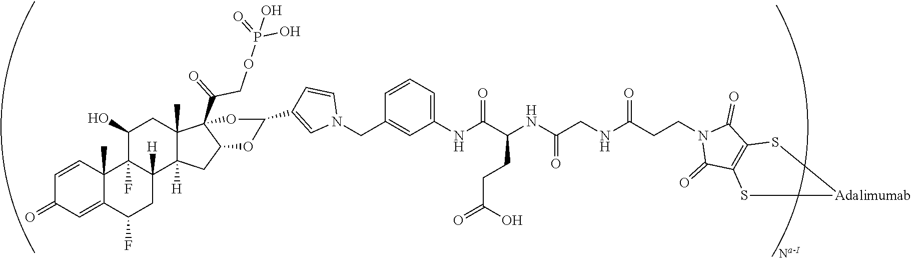 |
| II-C-241 | 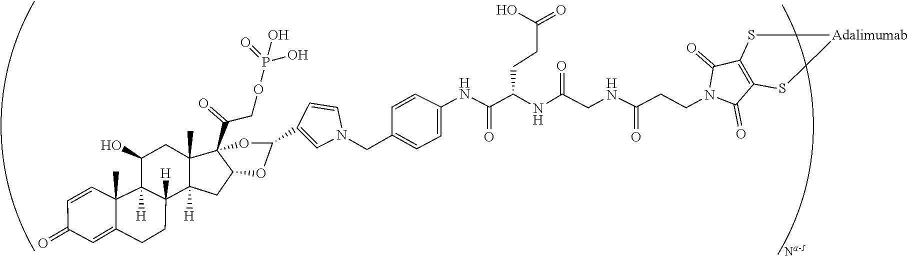 |
| II-C-242 | 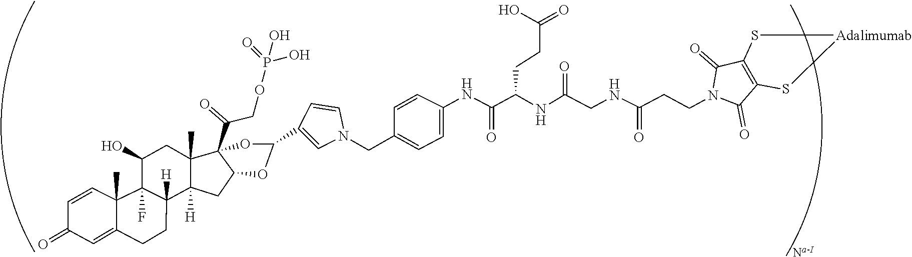 |
| II-C-243 | 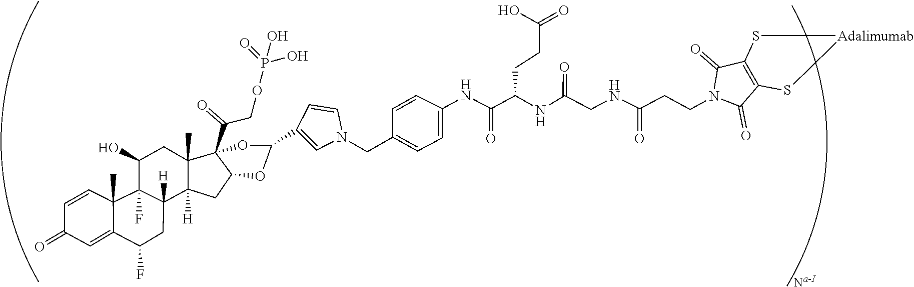 |
| II-C-244 | 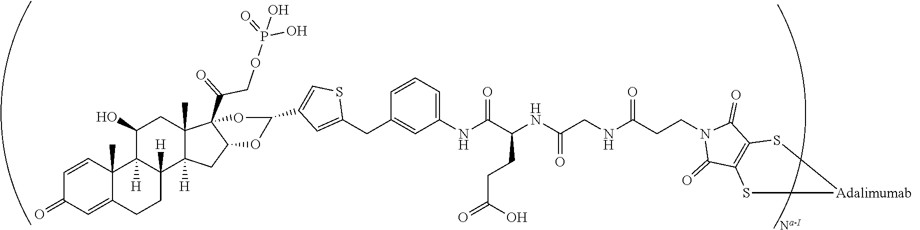 |
| II-C-245 | 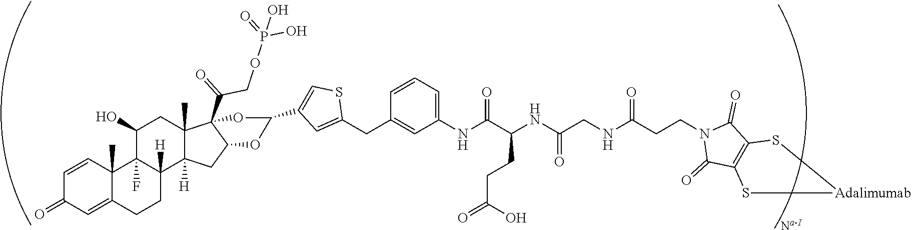 |
| II-C-246 | 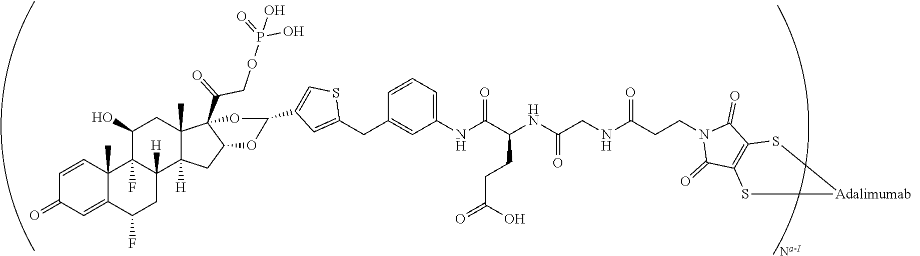 |
| II-C-247 | 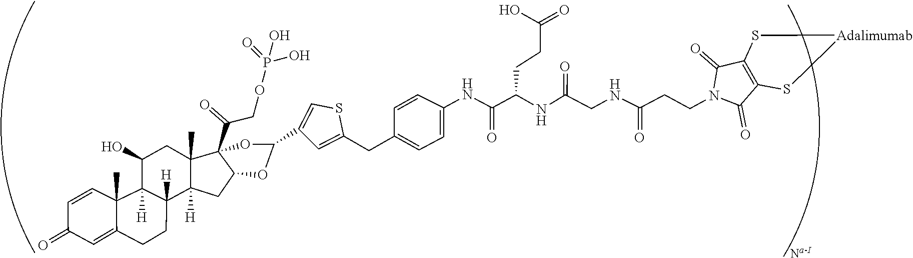 |
| II-C-248 | 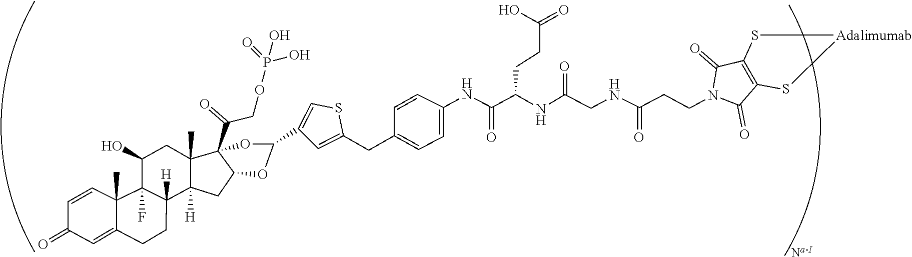 |
| II-C-249 | 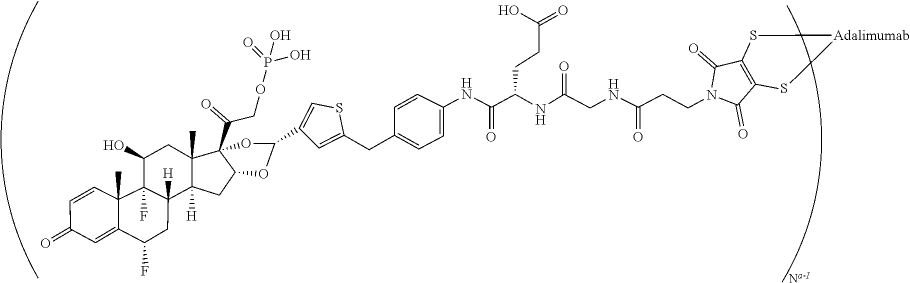 |
| II-C-250 | 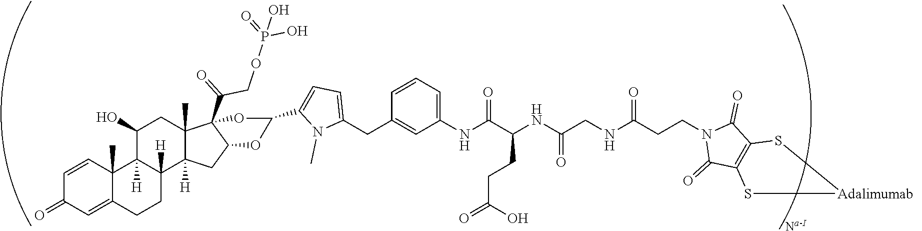 |
| II-C-253 | 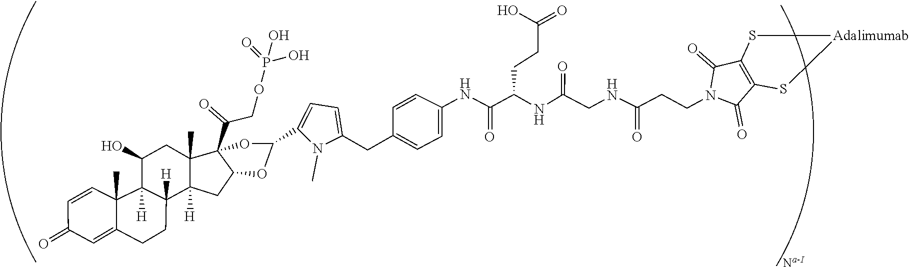 |
| II-C-256 | 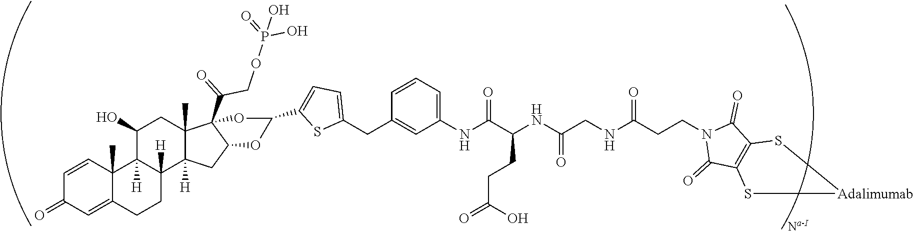 |
| II-C-257 | 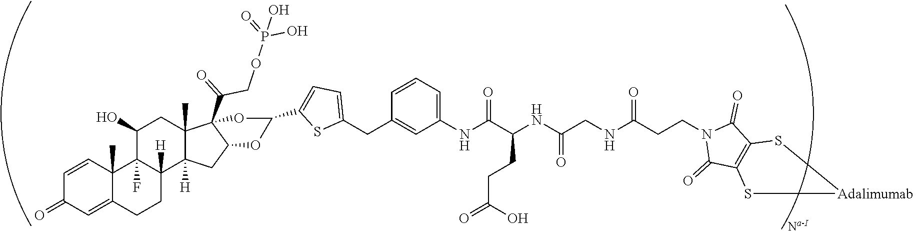 |
| II-C-258 | 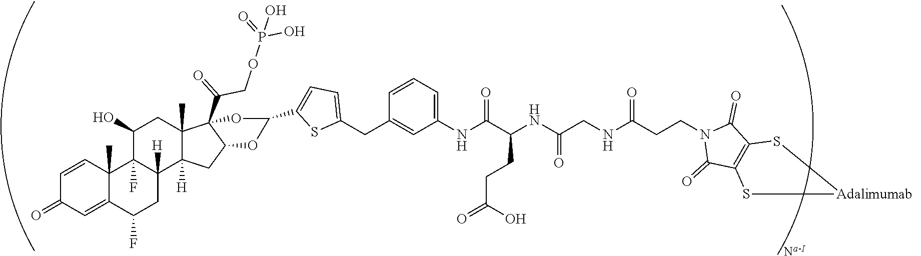 |
| II-C-259 | 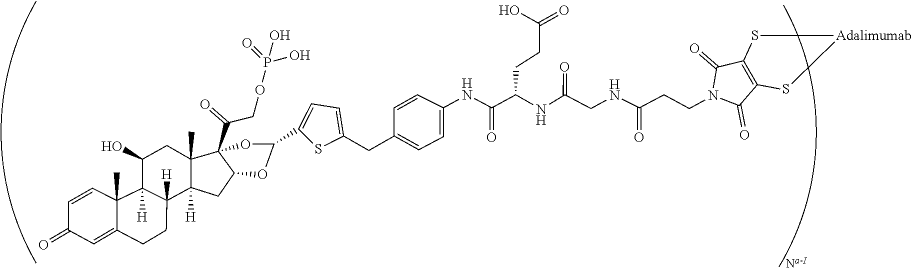 |
| II-C-260 | 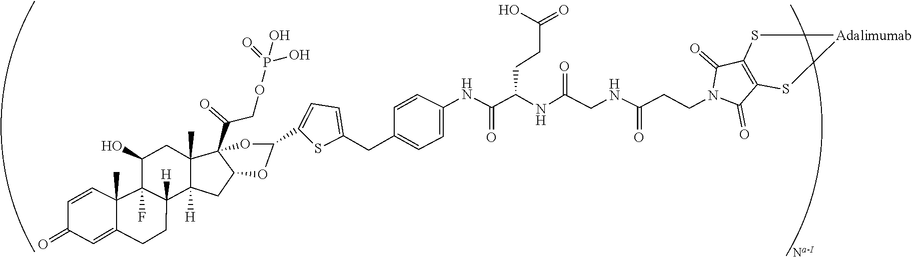 |
| II-C-261 | 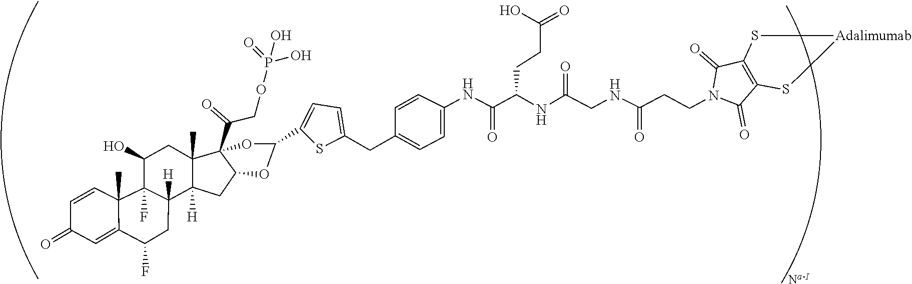 |
| II-C-262 | 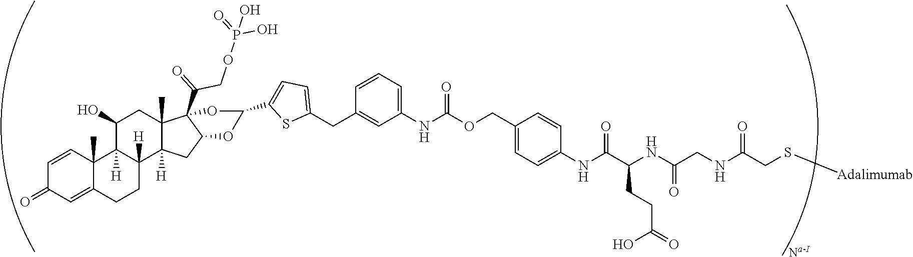 |
| II-C-263 | 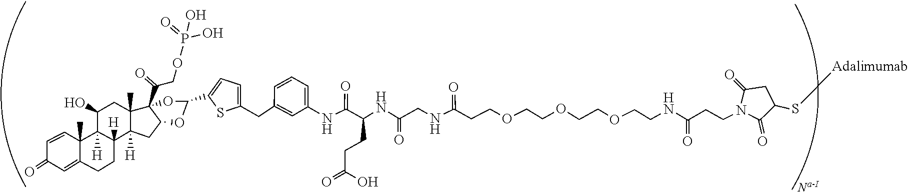 |
| II-C-264 | 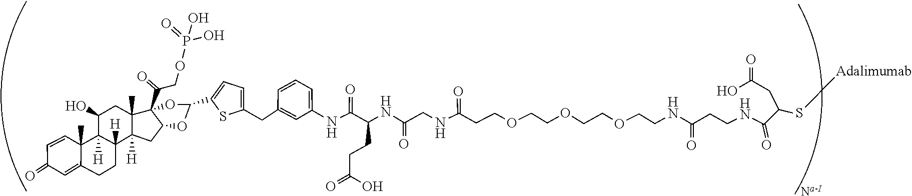 |
| II-C-265 | 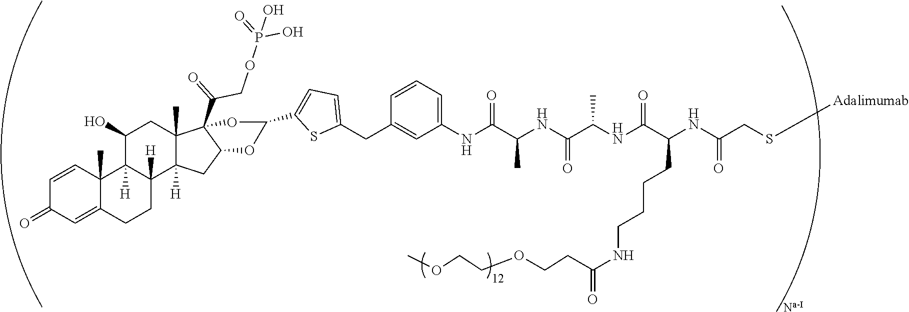 |
| II-C-266 | 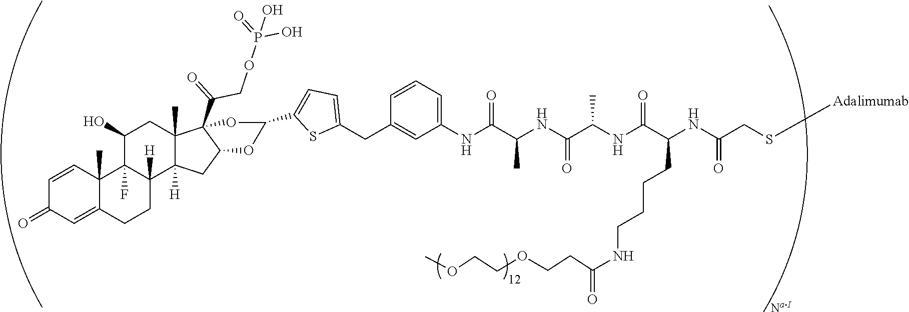 |
| II-C-267 | 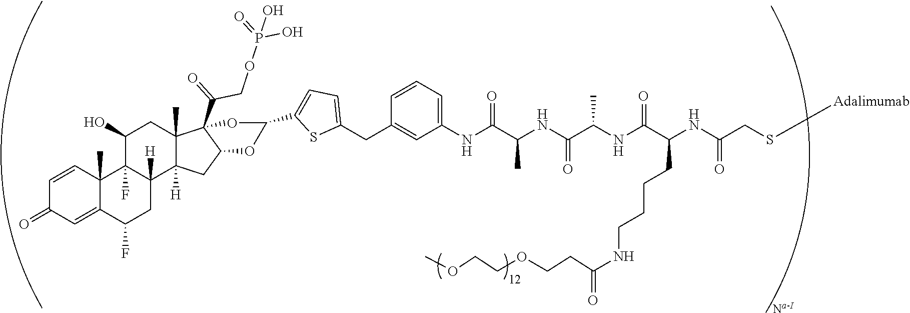 |
| II-C-268 | 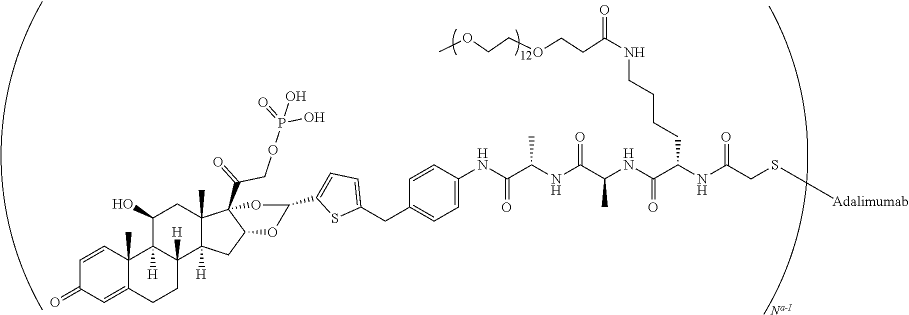 |
| II-C-269 | 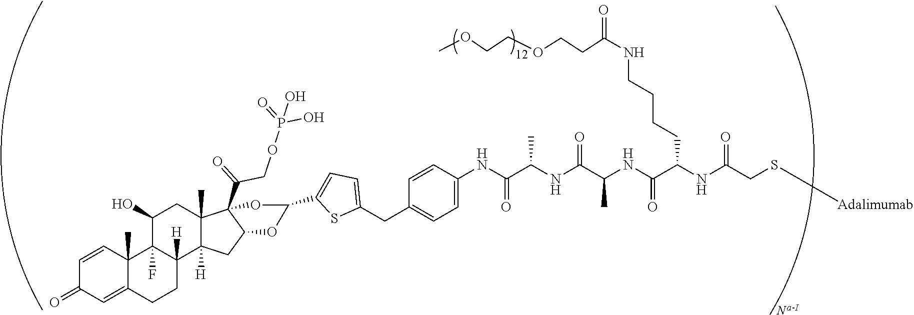 |
| II-C-270 | 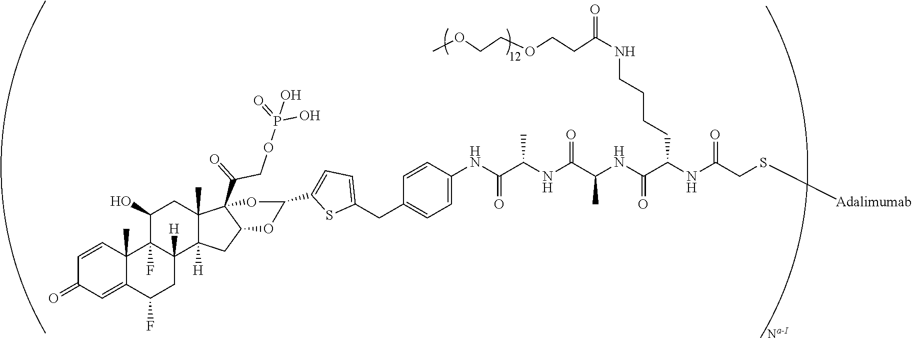 |
| II-C-271 | 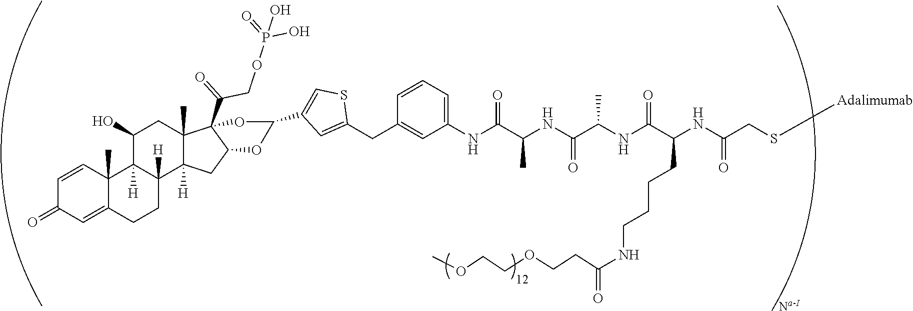 |
| II-C-272 | 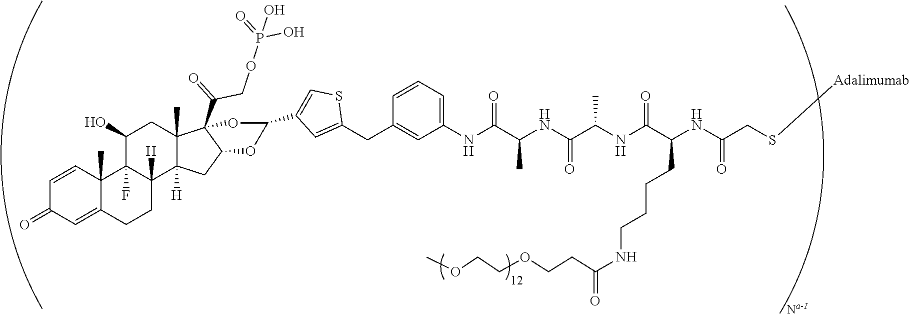 |
| II-C-273 | 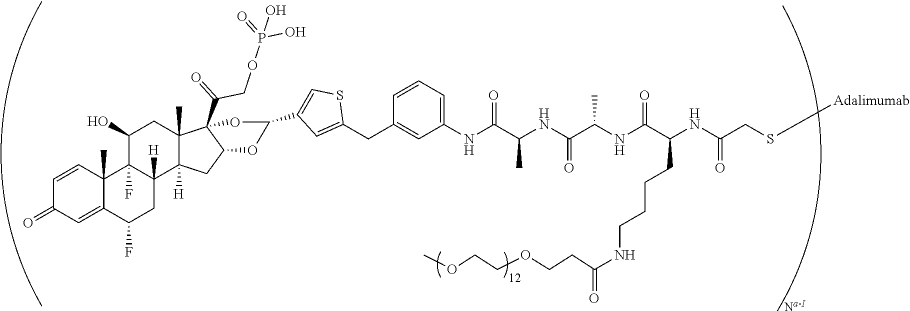 |
| II-C-274 | 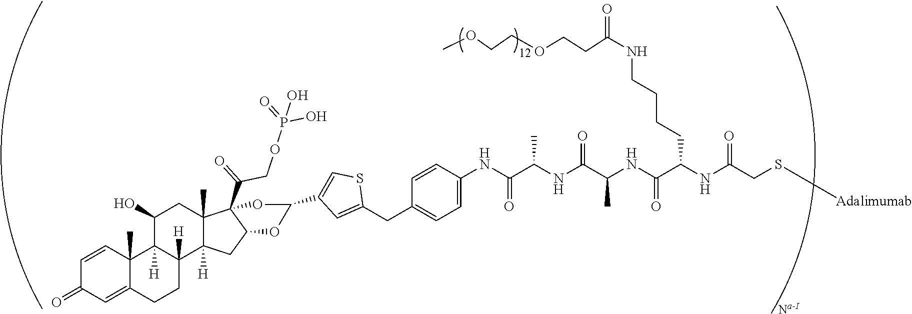 |
| II-C-275 | 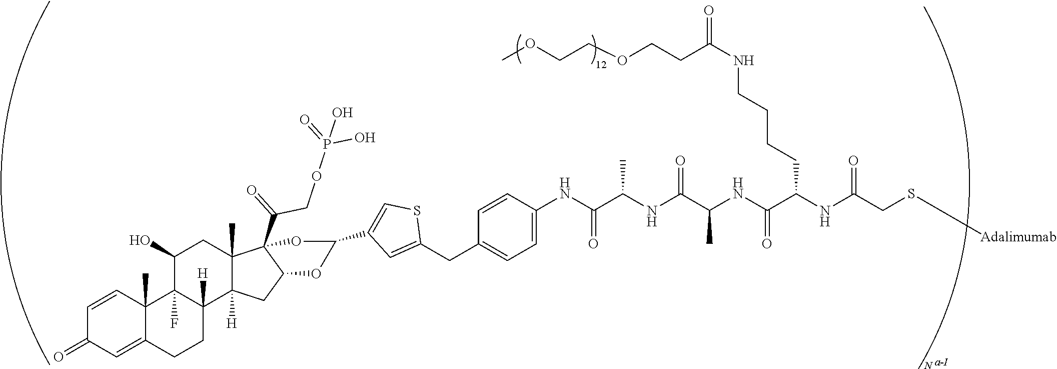 |
| II-C-276 | 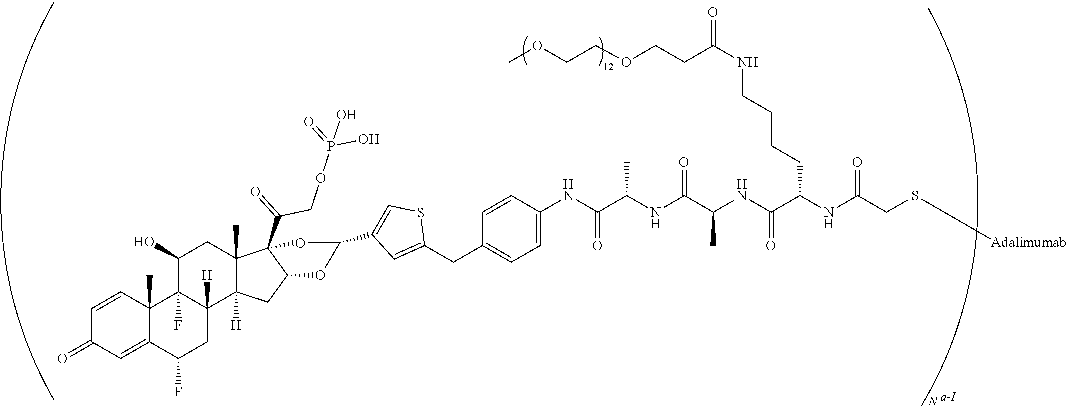 |
| II-C-277 | 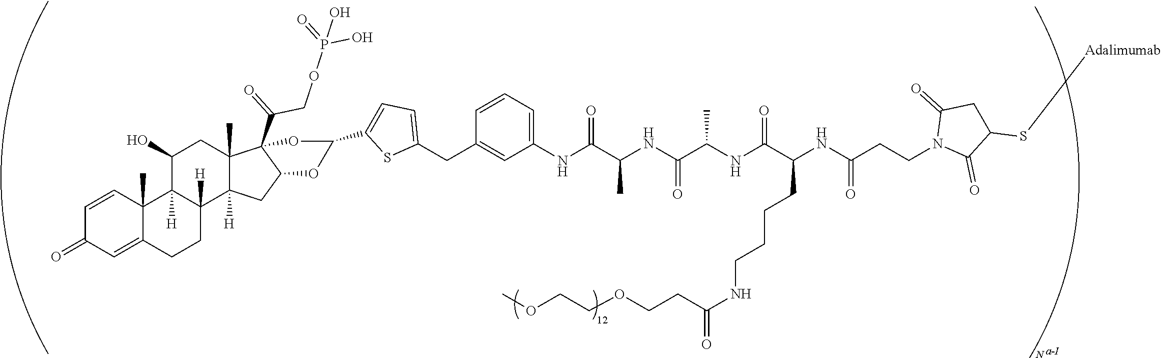 |
| II-C-278 | 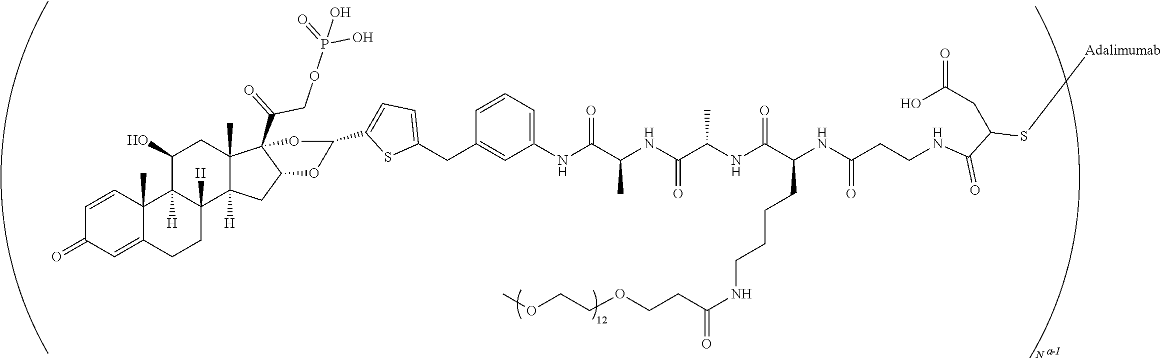 |
| II-C-279 | 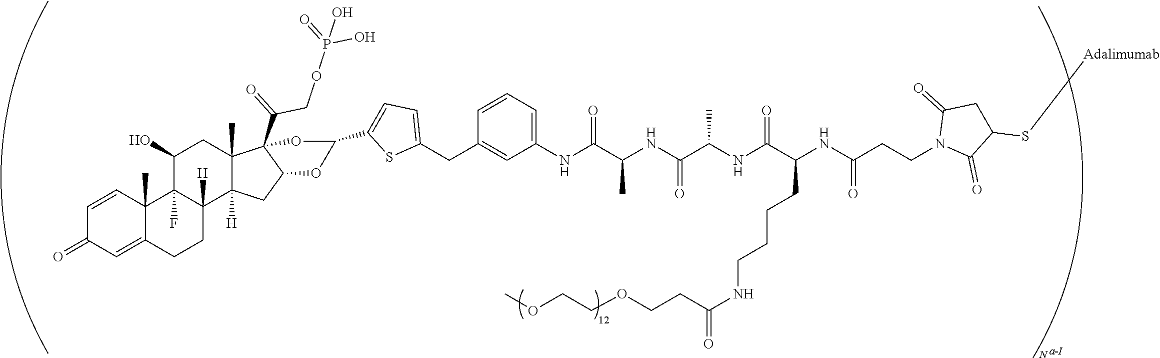 |
| II-C-280 | 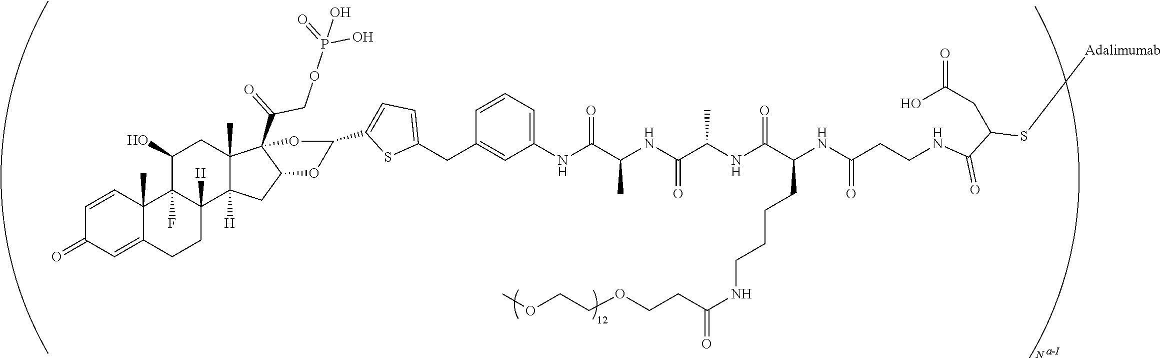 |
| II-C-281 | 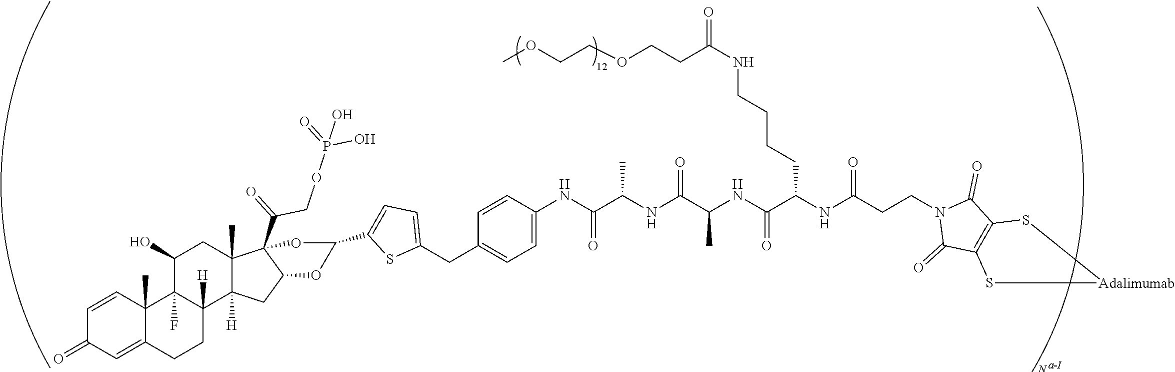 |
| II-C-282 | 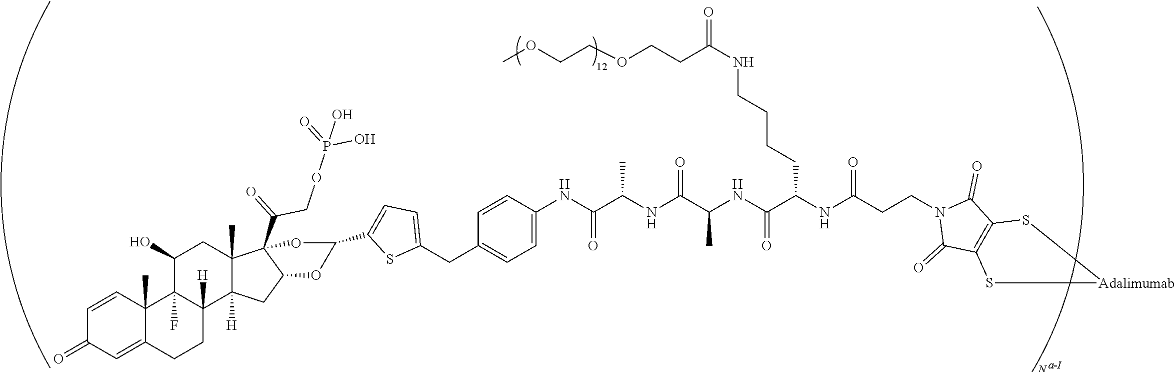 |
| II-C-283 | 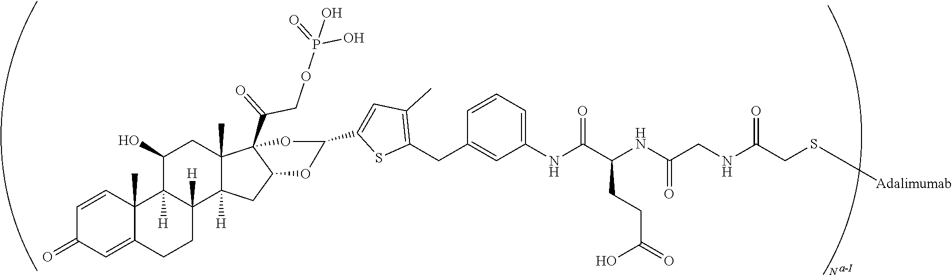 |
| II-C-286 | 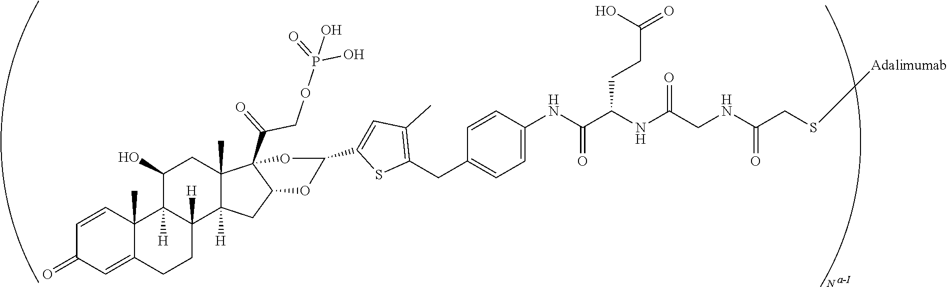 |
| II-C-289 | 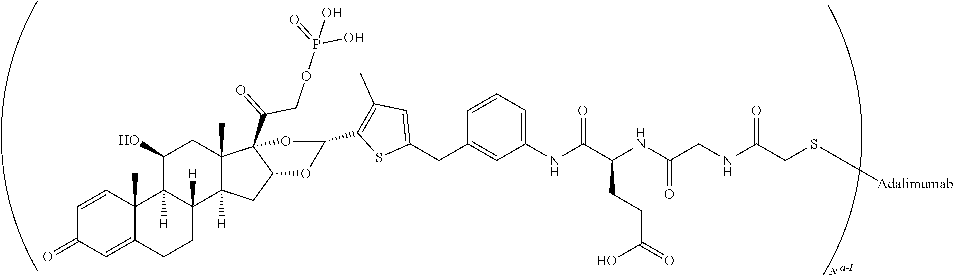 |
| II-C-292 | 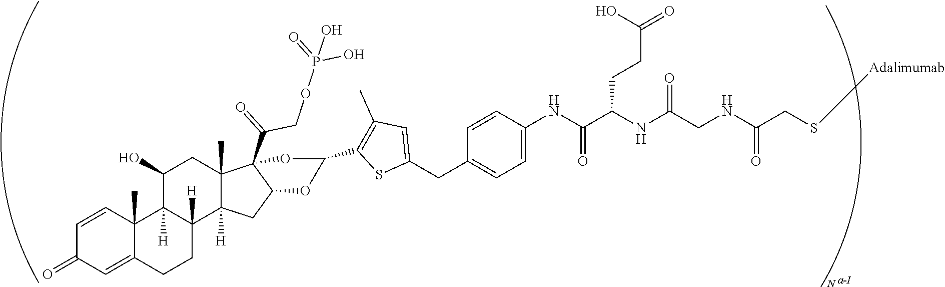 |
| II-C-295 | 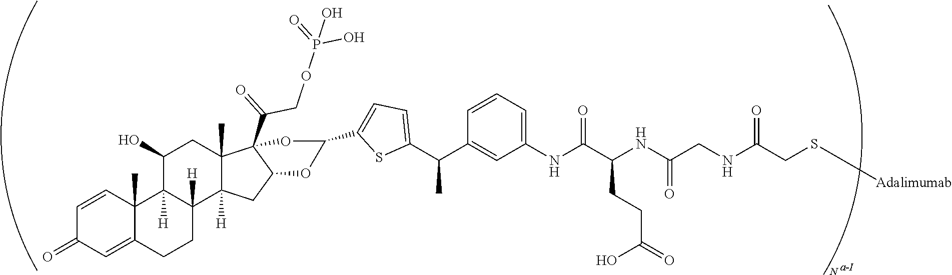 |
| II-C-298 | 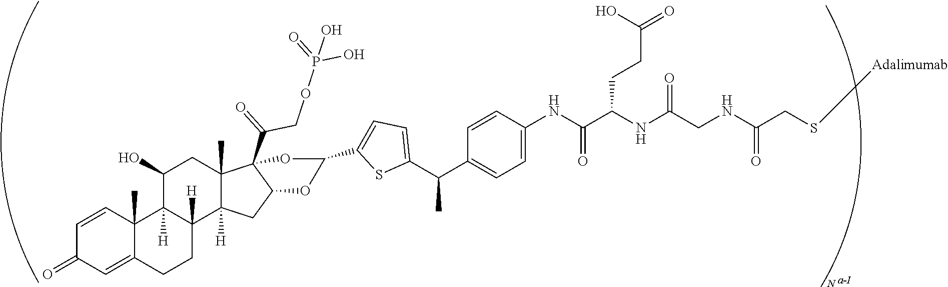 |
| II-C-301 | 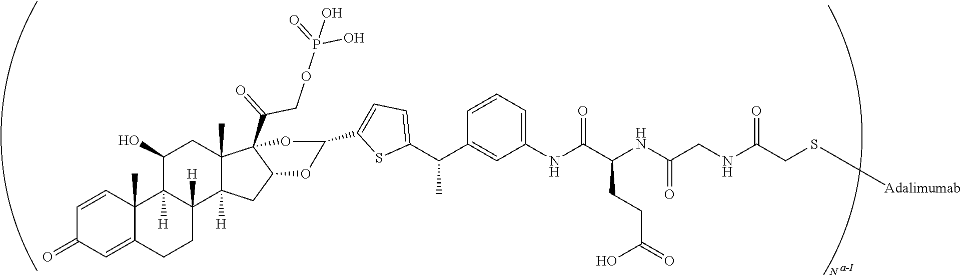 |
| II-C-304 | 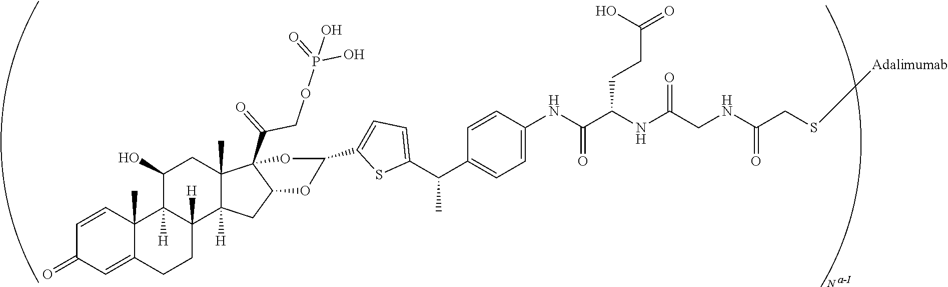 |
| II-C-307 | 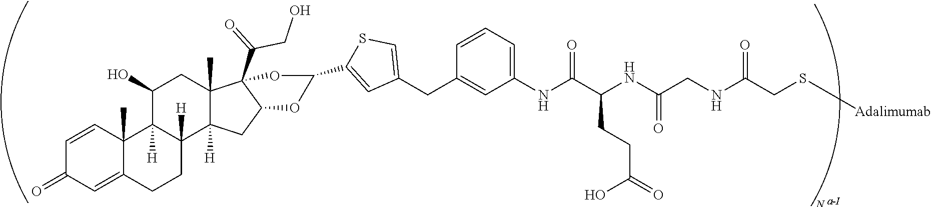 |
| II-C-308 | 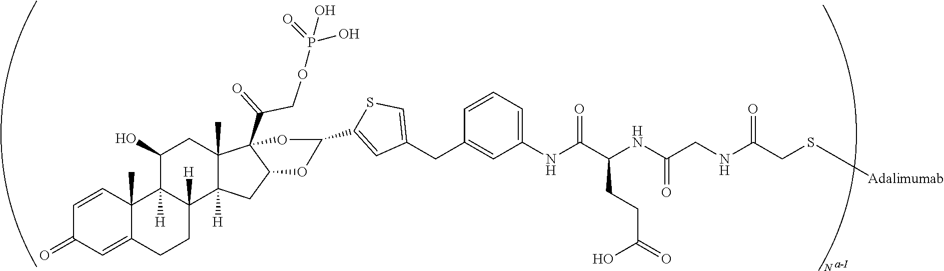 |
| II-C-312 | 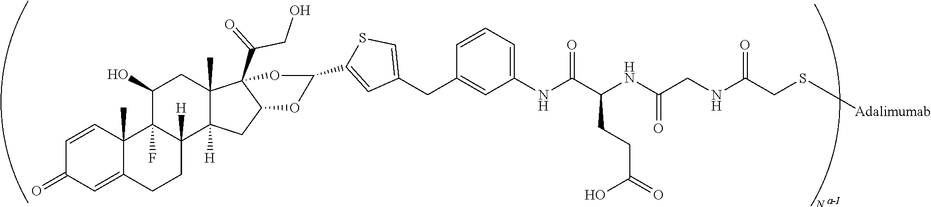 |
| II-C-313 | 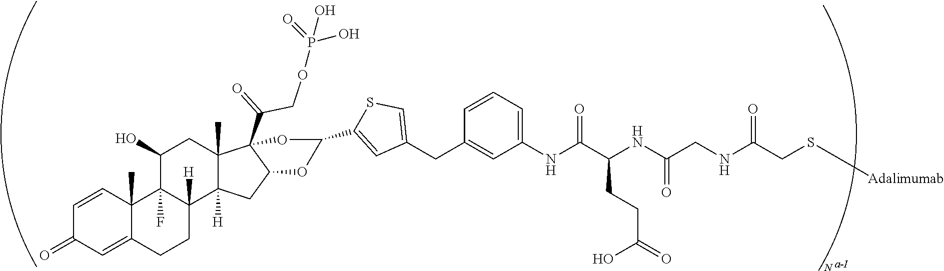 |
| II-C-317 | 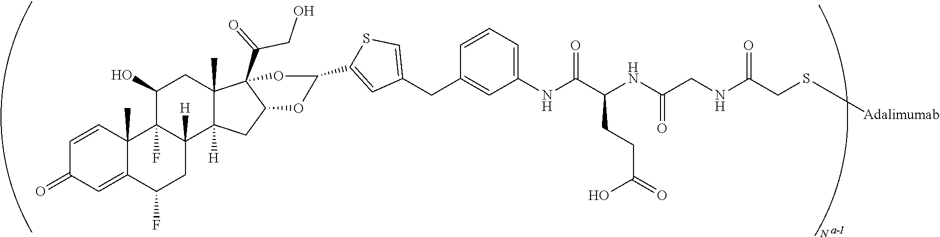 |
| II-C-318 | 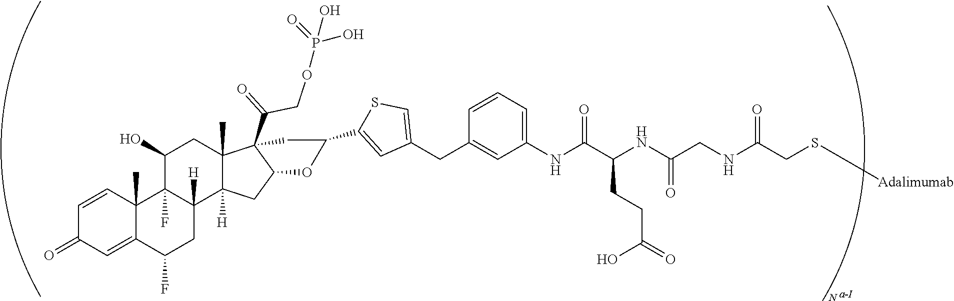 |
| II-C-322 | 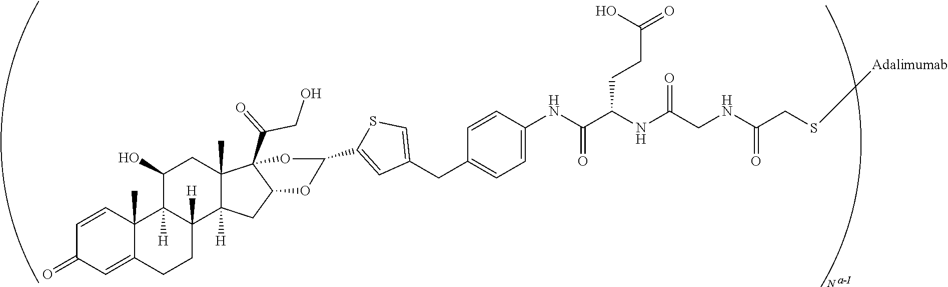 |
| II-C-323 | 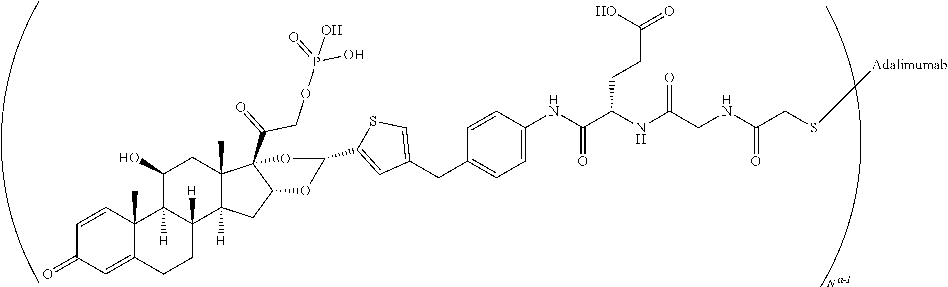 |
| II-C-327 | 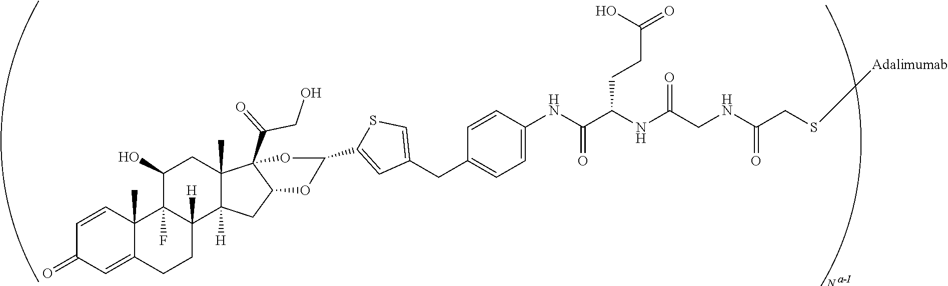 |
| II-C-328 | 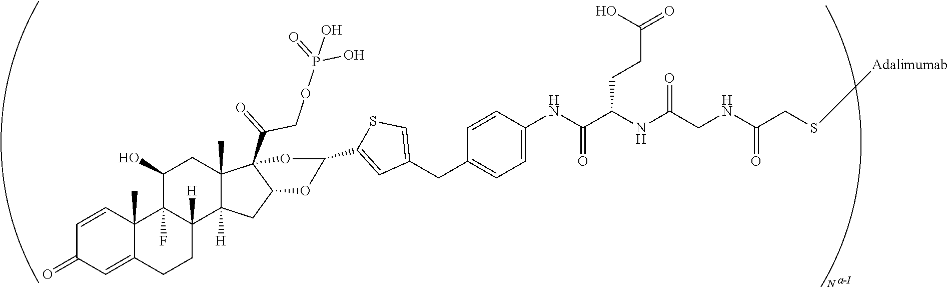 |
| II-C-332 | 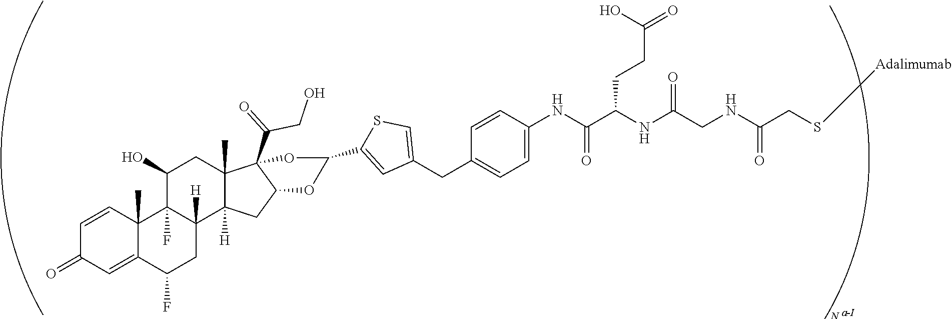 |
| II-C-333 | 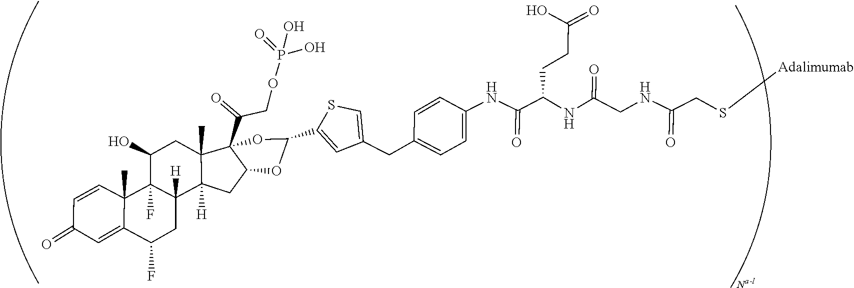 |
| II-C-337 | 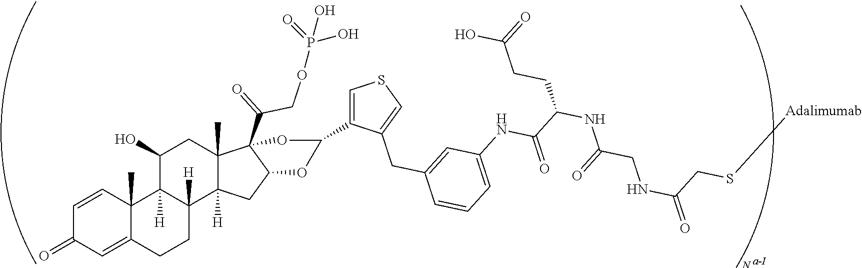 |
| II-C-338 | 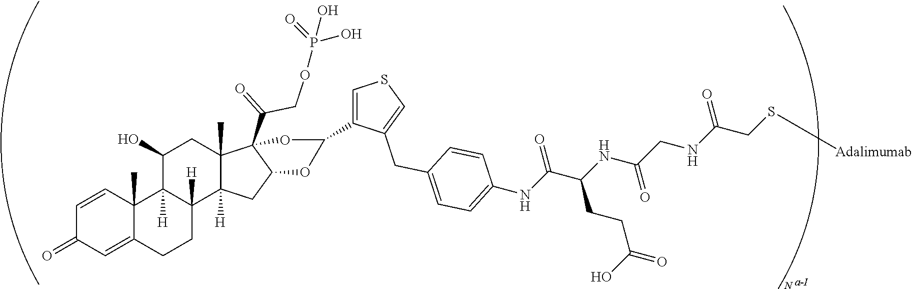 |
| II-C-339 | 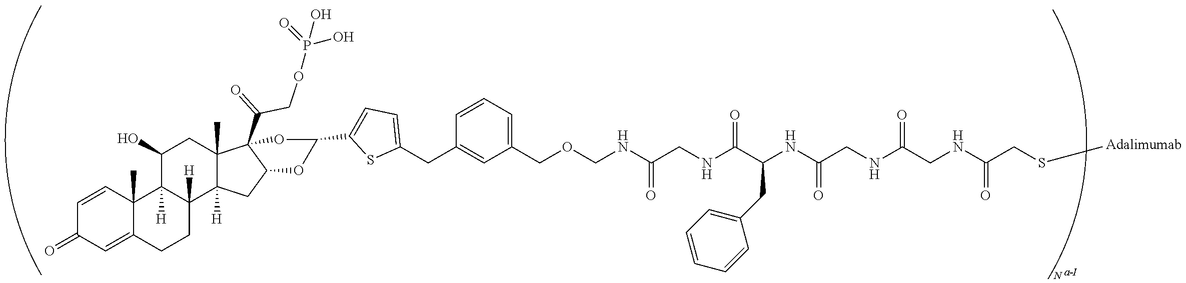 |
| II-C-340 | 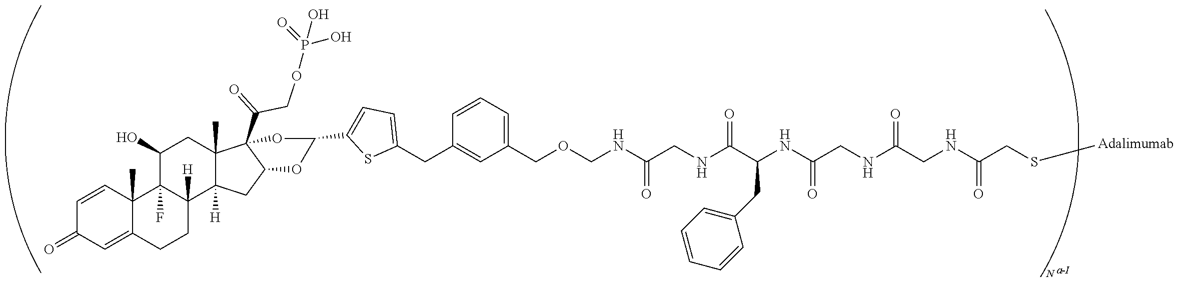 |
| II-C-341 | 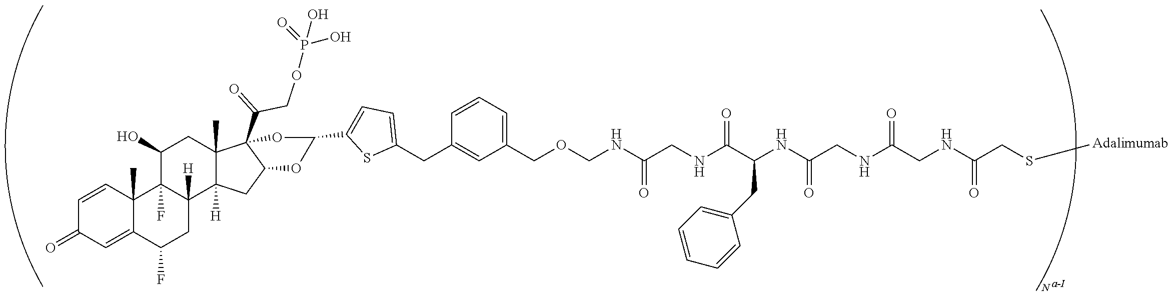 |
| II-C-342 | 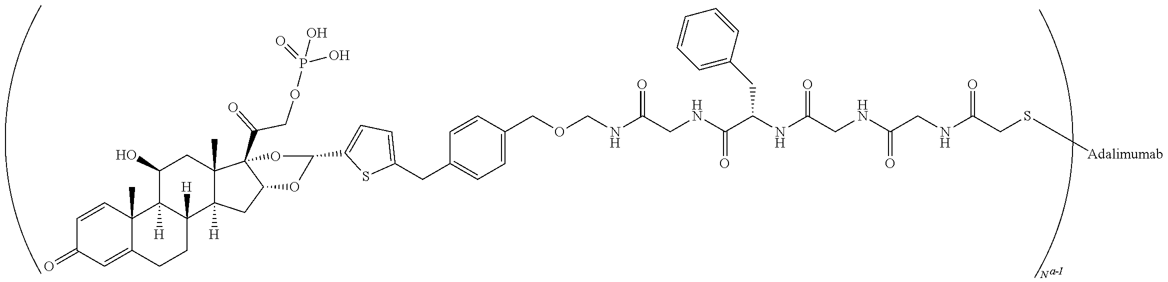 |
| II-C-343 | 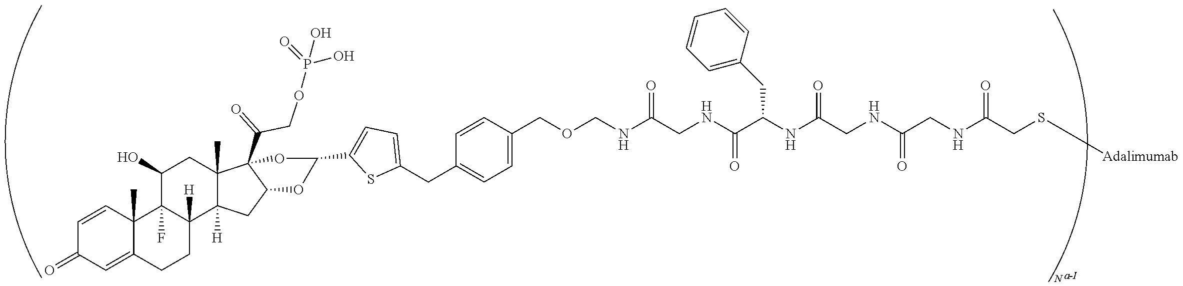 |
| II-C-344 | 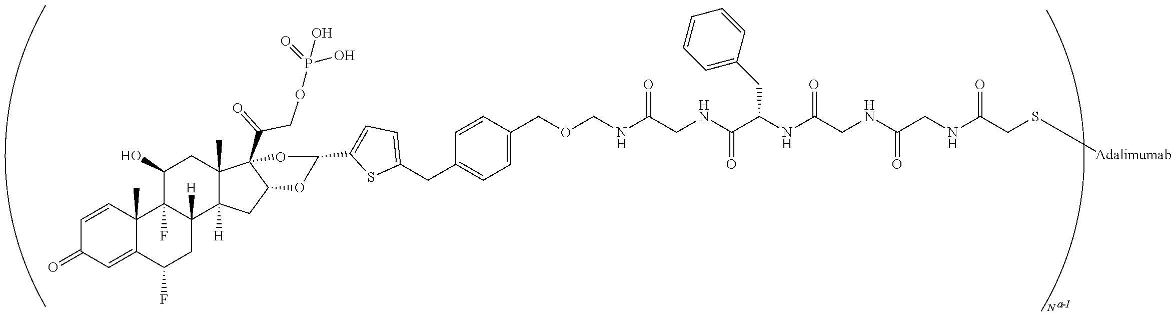 |
| II-C-345 | 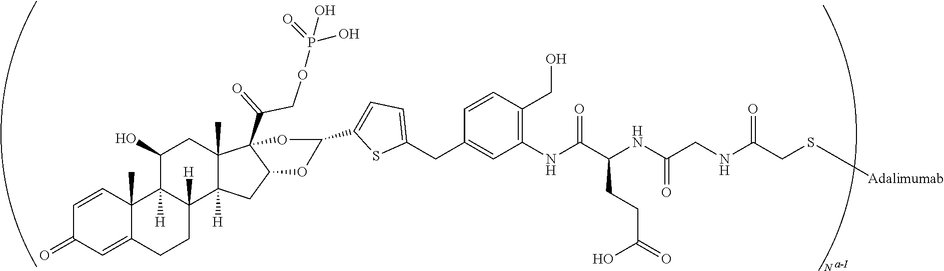 |
| II-C-348 | 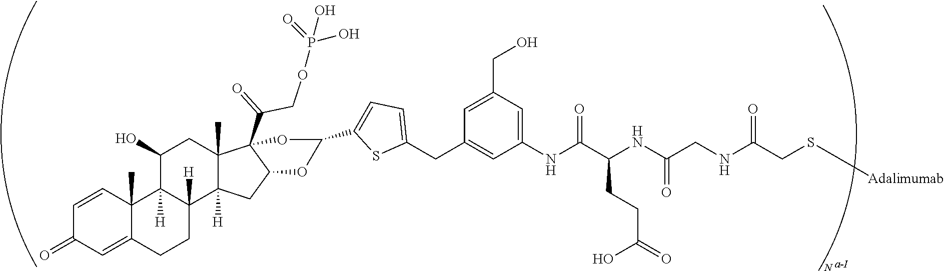 |
| II-C-351 | 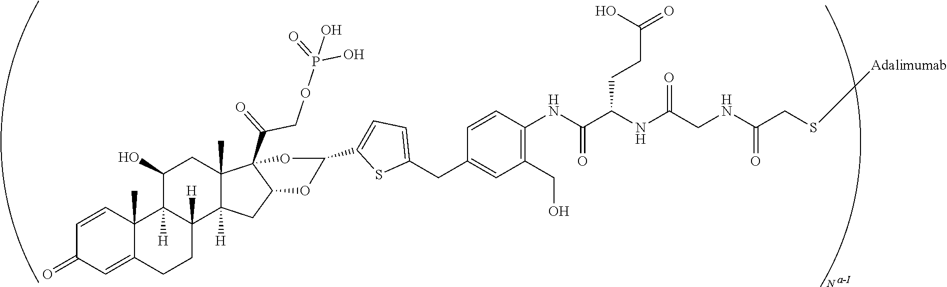 |
| II-C-354 | 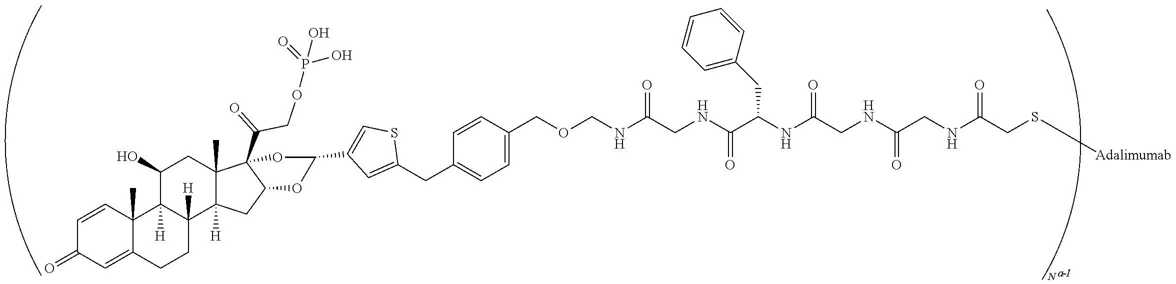 |
| II-C-355 | 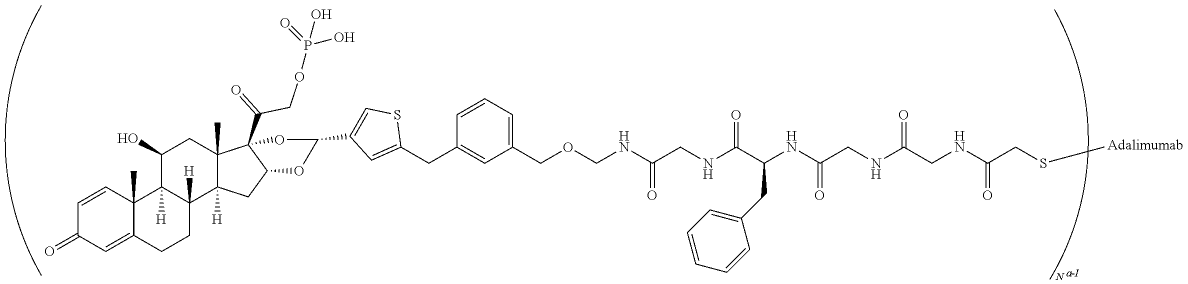 |
| II-C-356 | 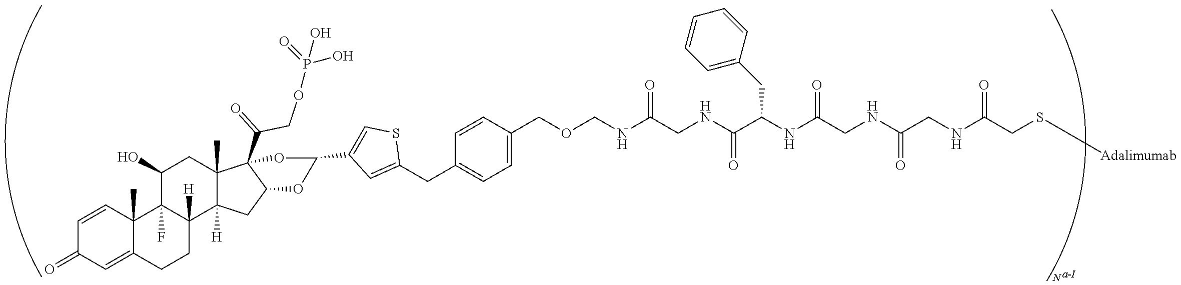 |
| II-C-357 | 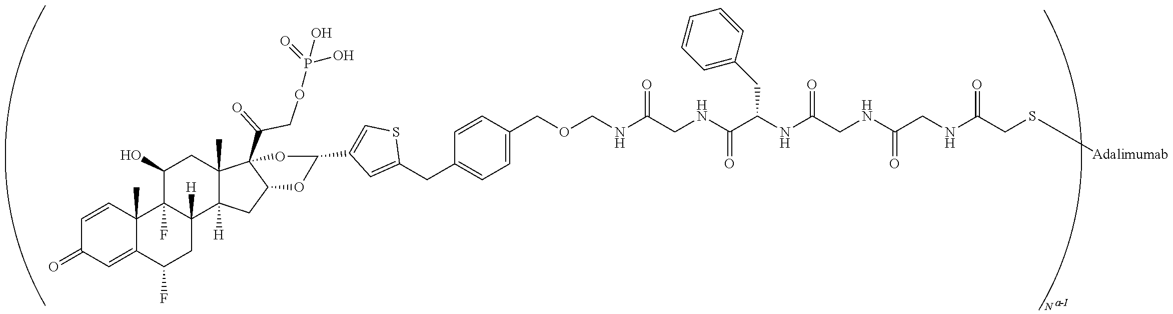 |
| II-C-358 | 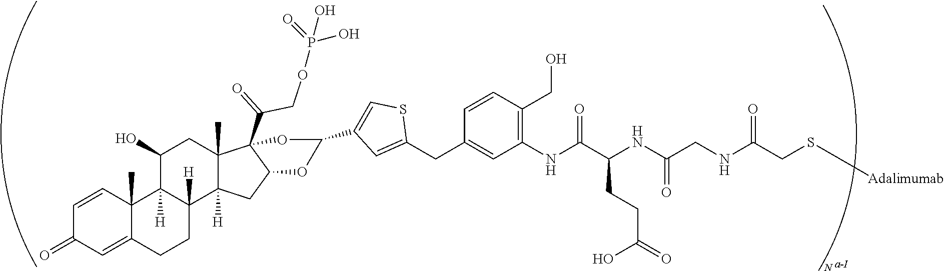 |
| II-C-361 | 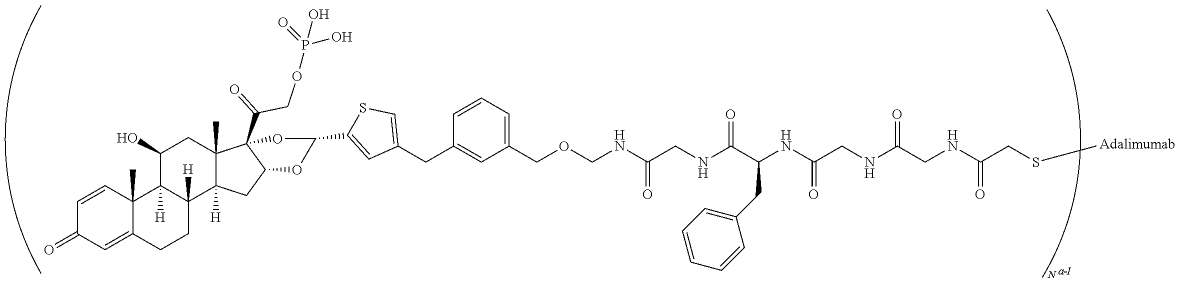 |
| II-C-362 | 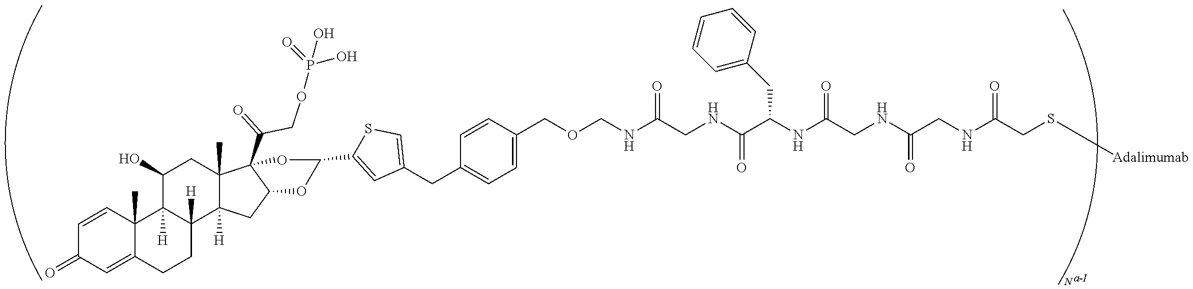 |
| II-C-363 | 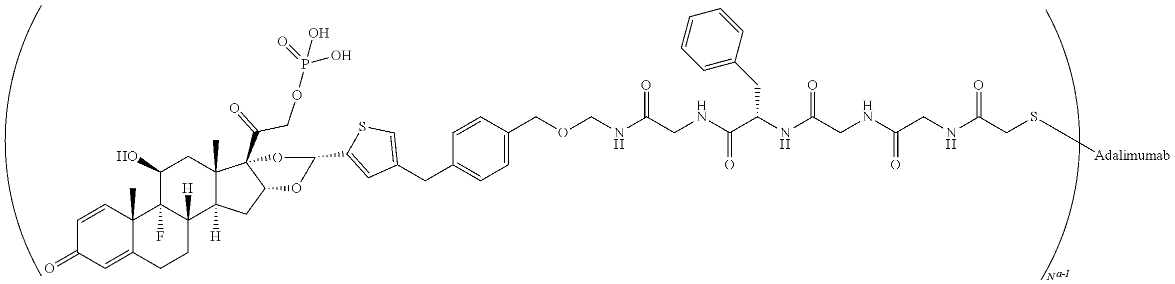 |
| II-C-364 | 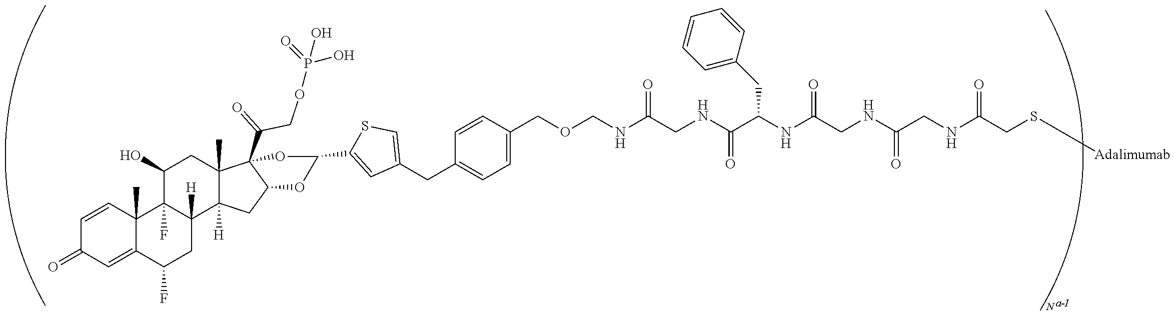 |
| II-C-365 | 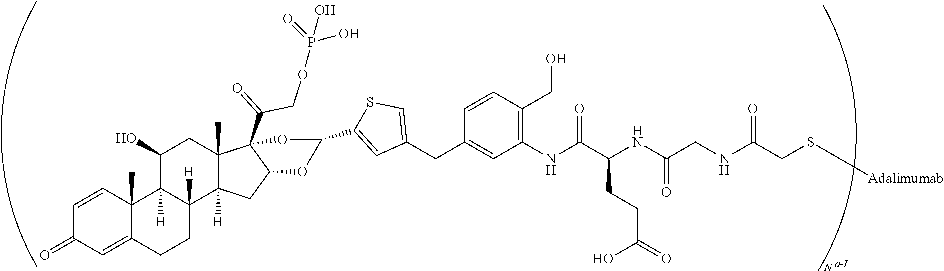 |
| II-C-368 | 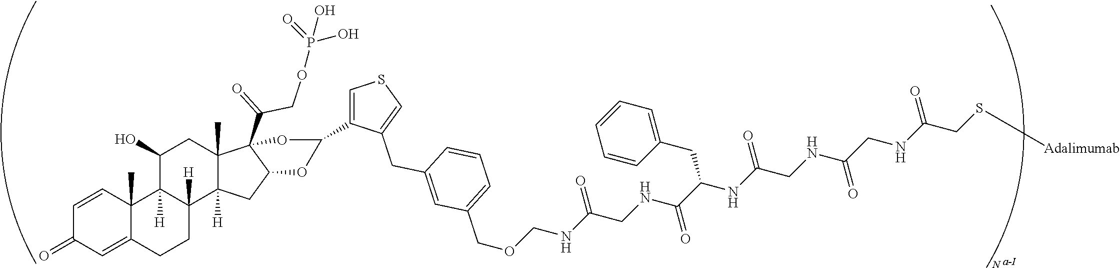 |
| II-C-369 | 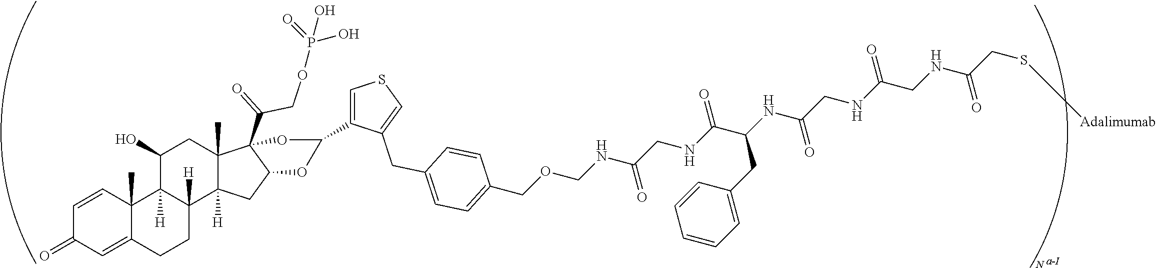 |
| II-C-385 | 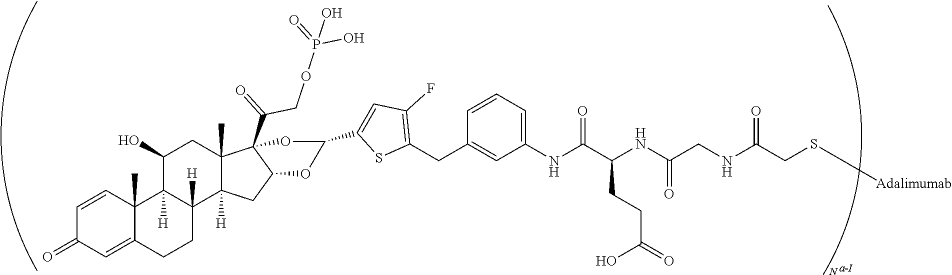 |
| II-C-388 | 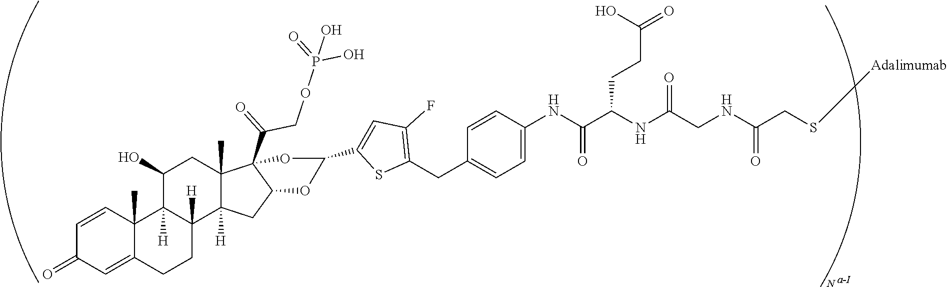 |
| II-C-391 | 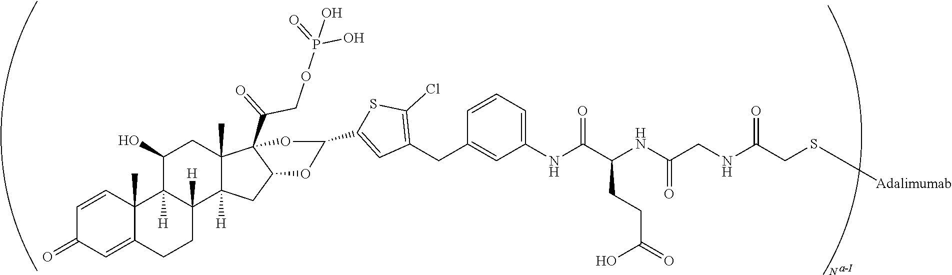 |
| II-C-394 | 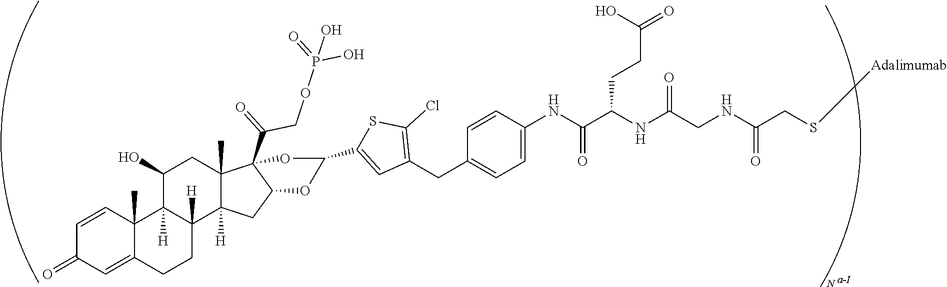 |
| II-C-397 | 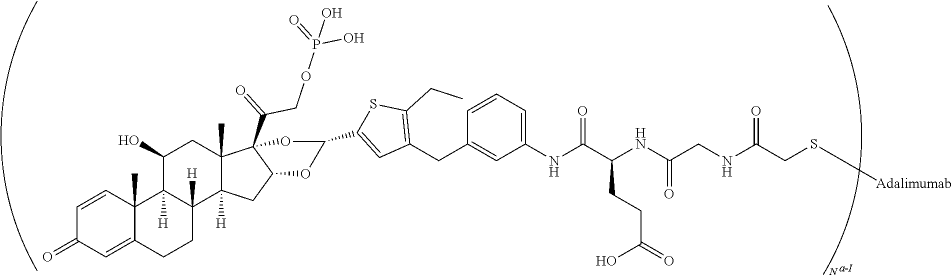 |
| II-C-400 | 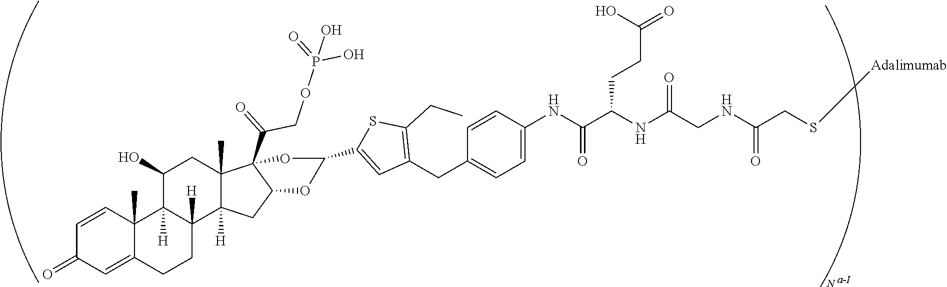 |
| II-C-403 | 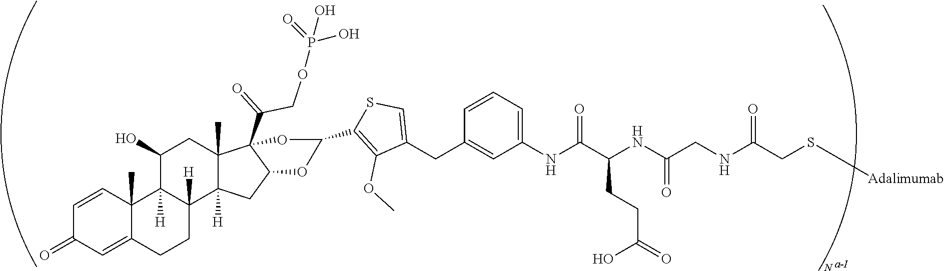 |
| II-C-406 | 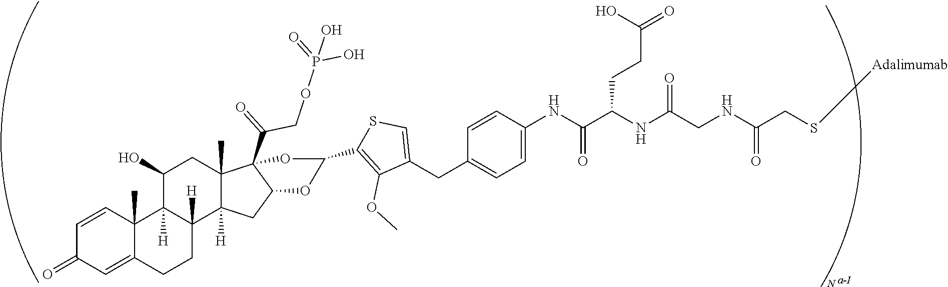 |
| II-C-409 | 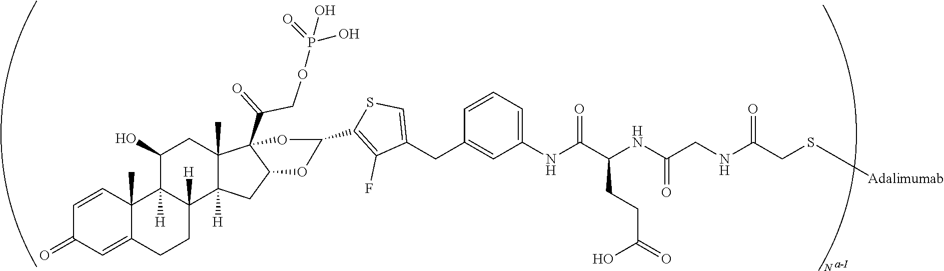 |
| II-C-412 | 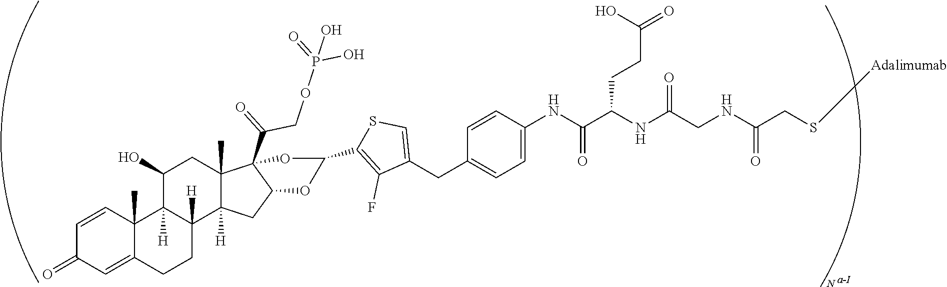 |
| II-C-415 | 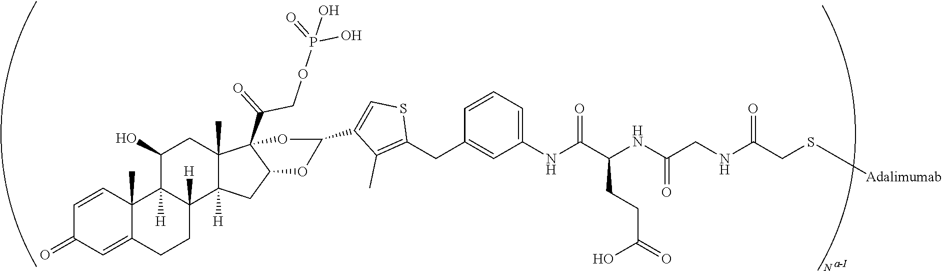 |
| II-C-418 | 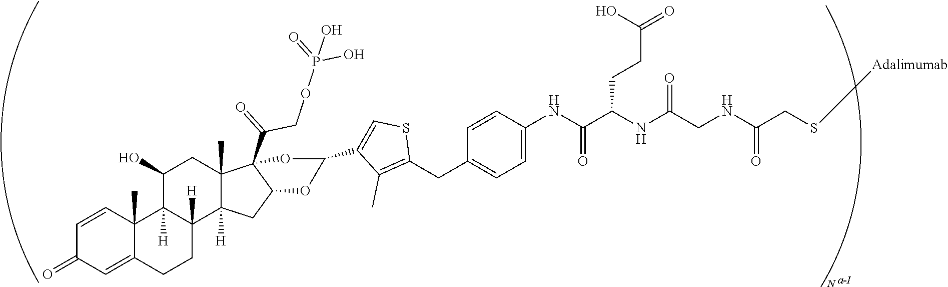 |
| II-C-421 | 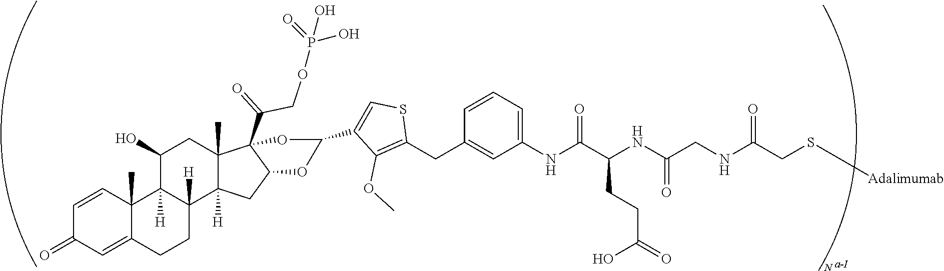 |
| II-C-424 | 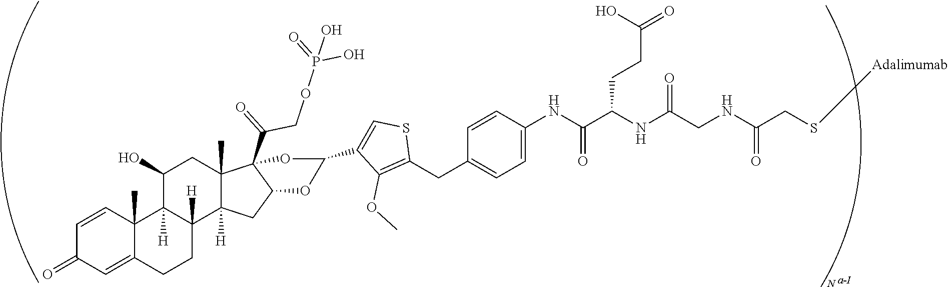 |
| II-C-427 | 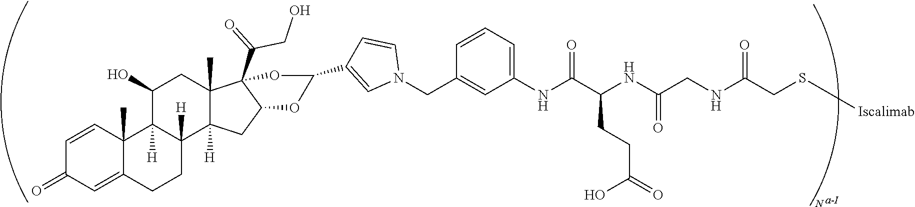 |
| II-C-428 | 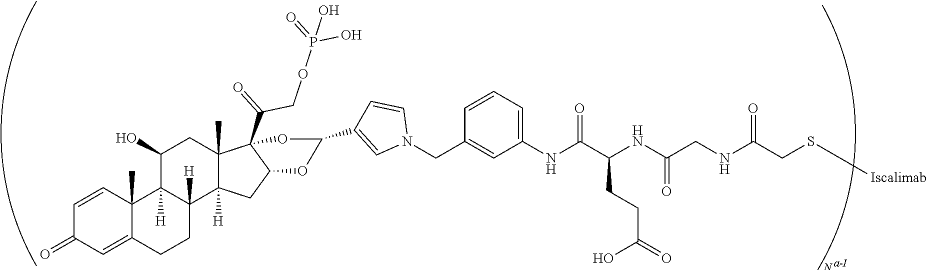 |
| II-C-433 | 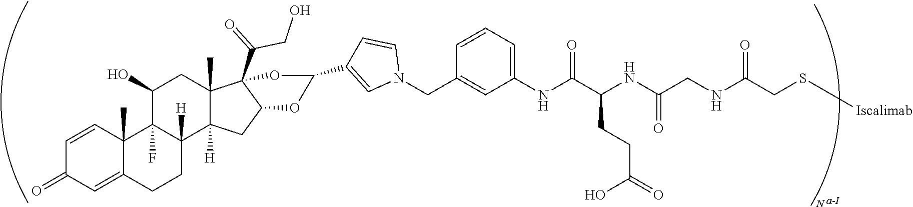 |
| II-C-434 | 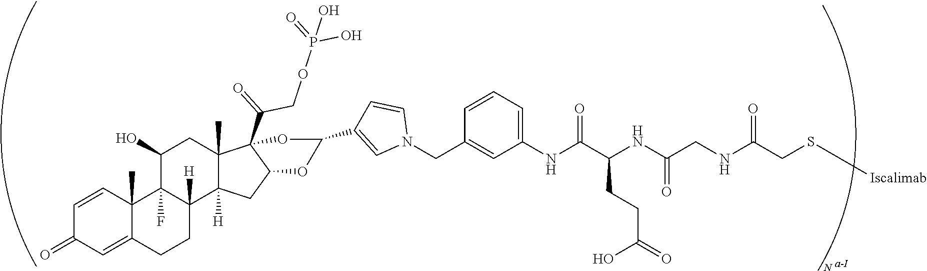 |
| II-C-438 | 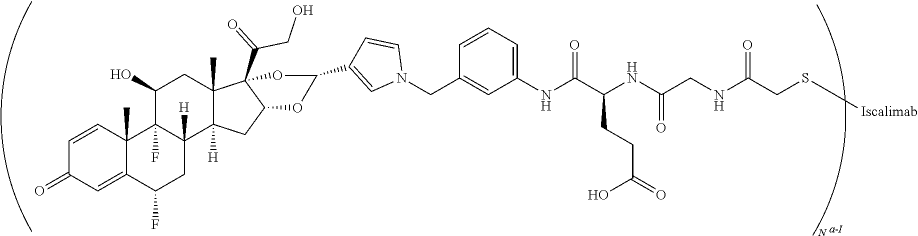 |
| II-C-439 | 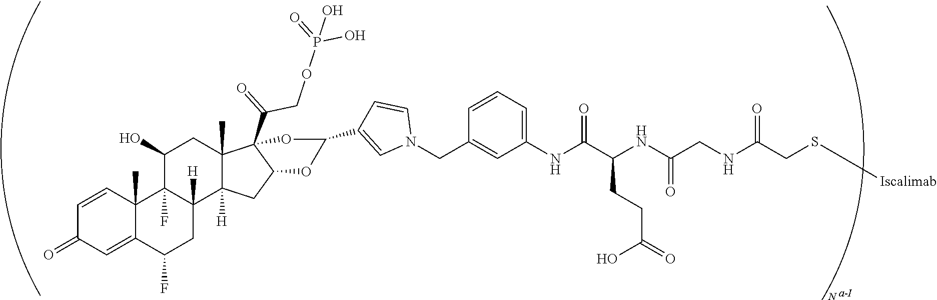 |
| II-C-443 | 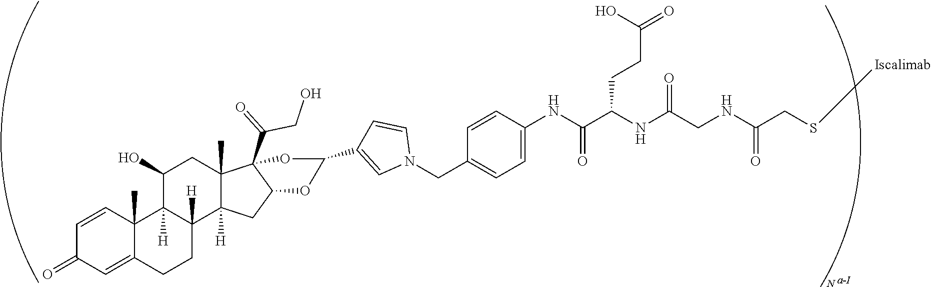 |
| II-C-444 | 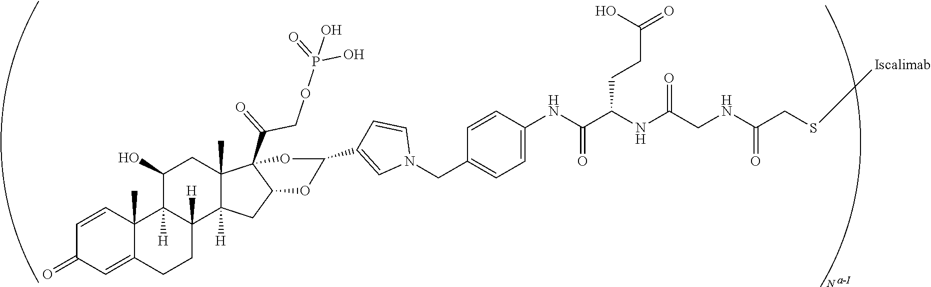 |
| II-C-449 | 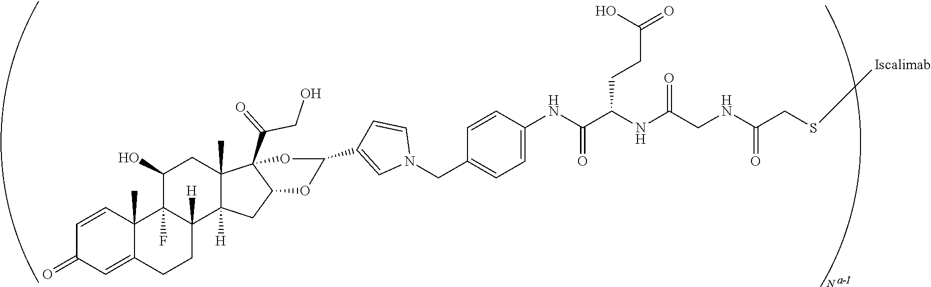 |
| II-C-450 | 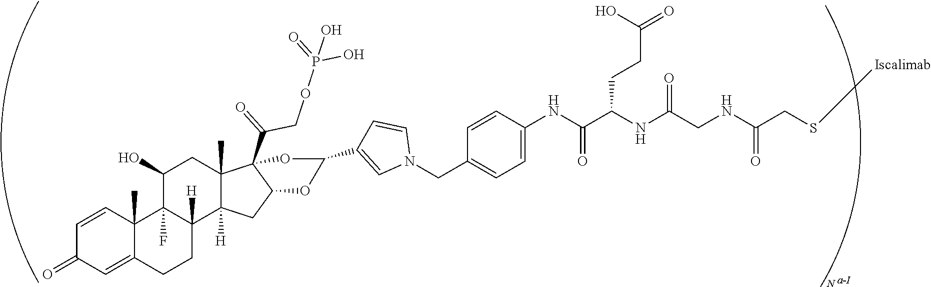 |
| II-C-455 | 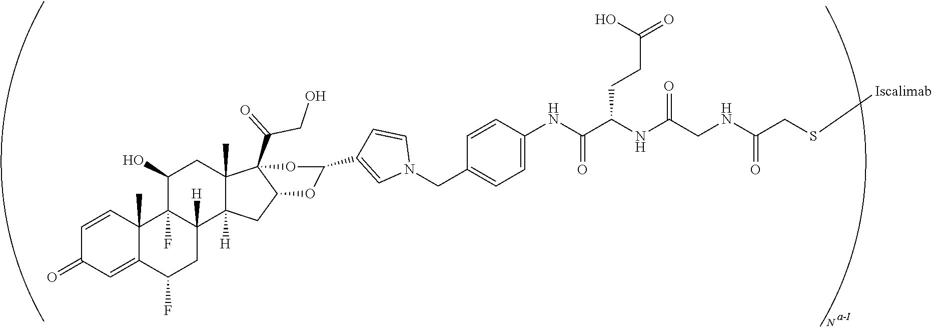 |
| II-C-456 | 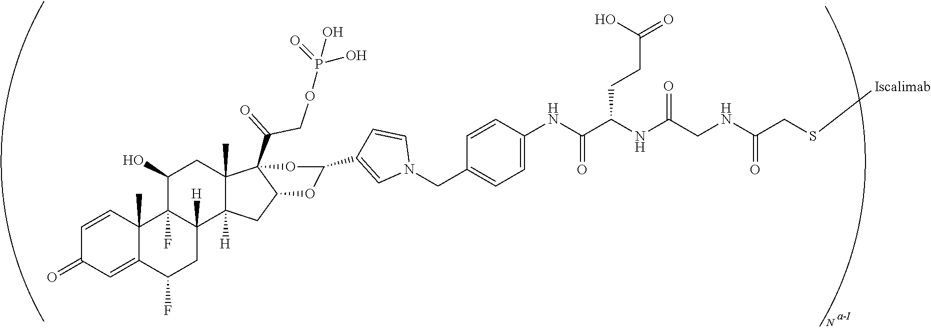 |
| II-C-460 | 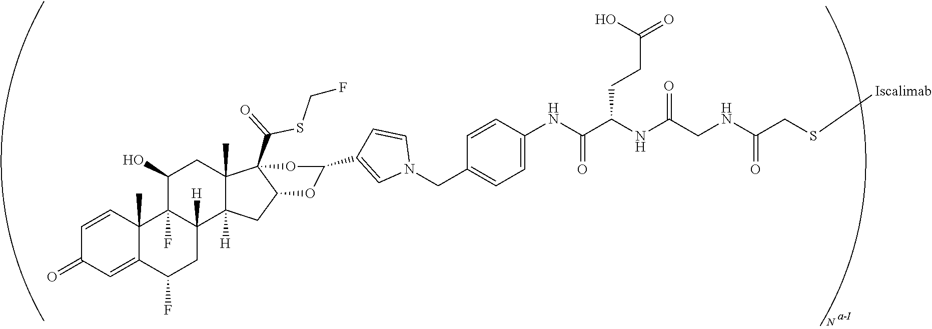 |
| II-C-461 | 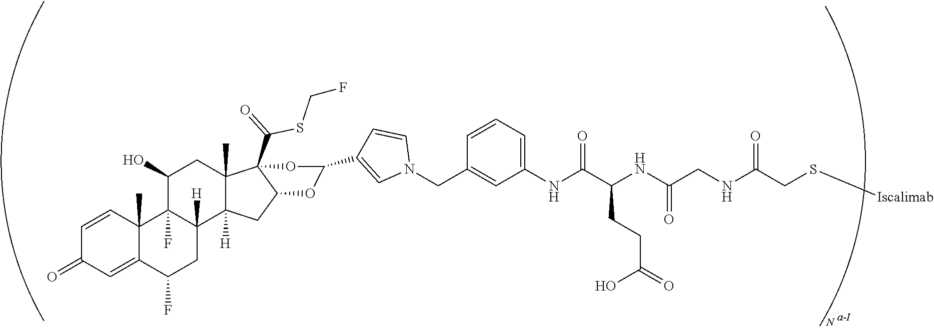 |
| II-C-537 | 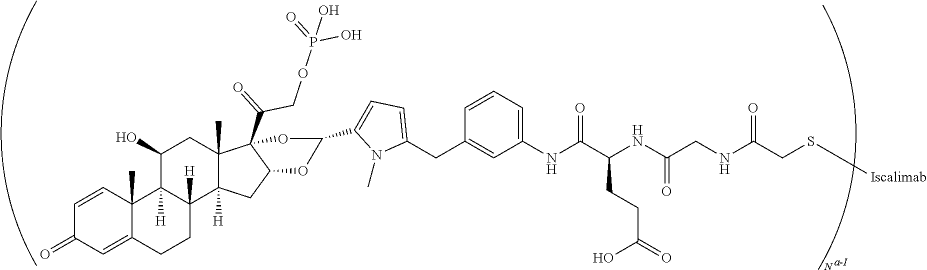 |
| II-C-540 | 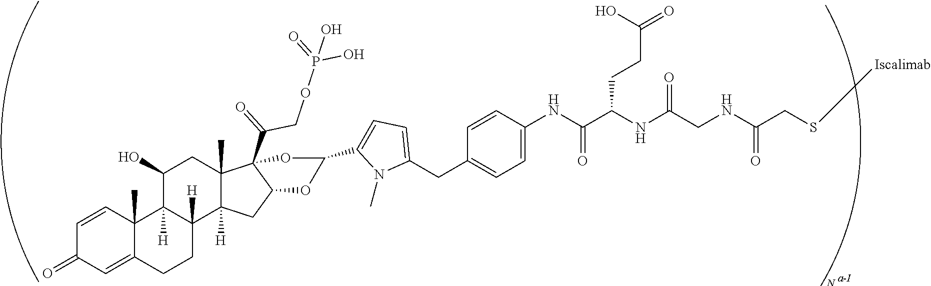 |
| II-C-543 | 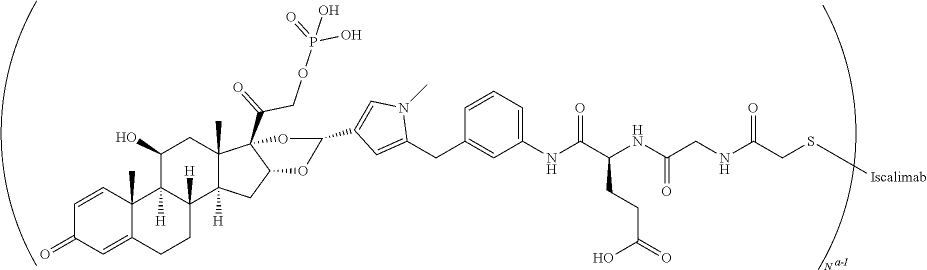 |
| II-C-546 | 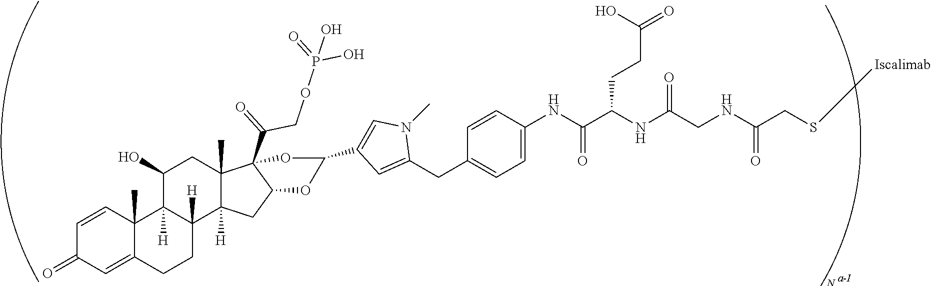 |
| II-C-549 | 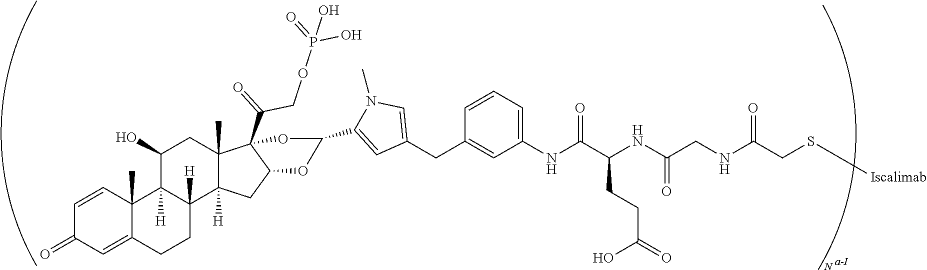 |
| II-C-552 | 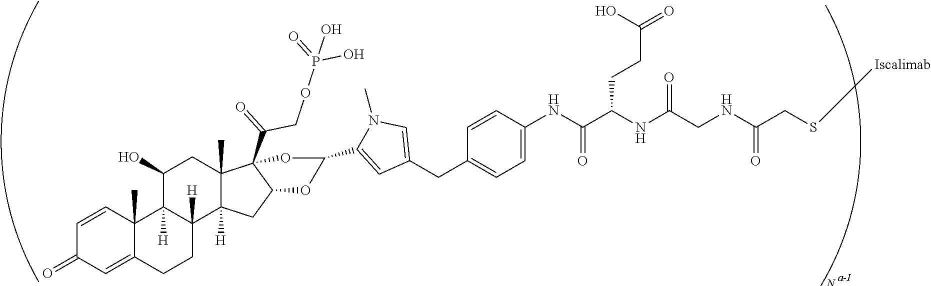 |
| II-C-592 | 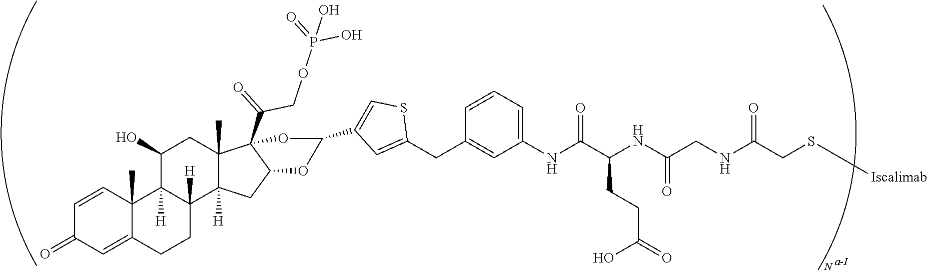 |
| II-C-597 | 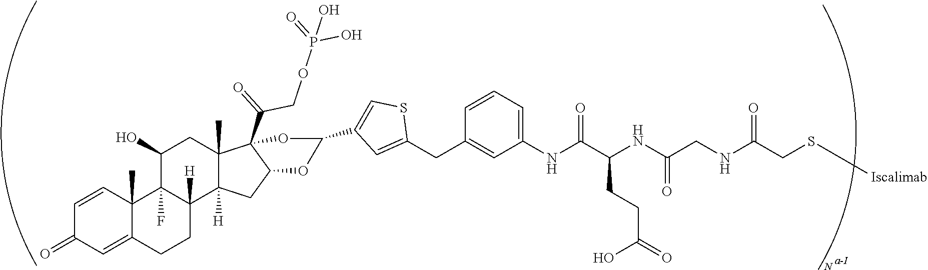 |
| II-C-602 | 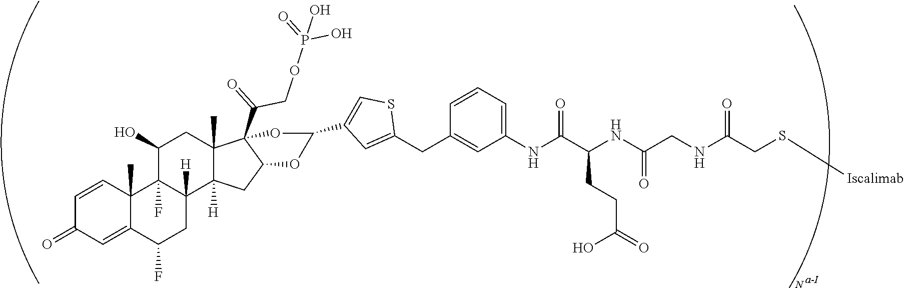 |
| II-C-607 | 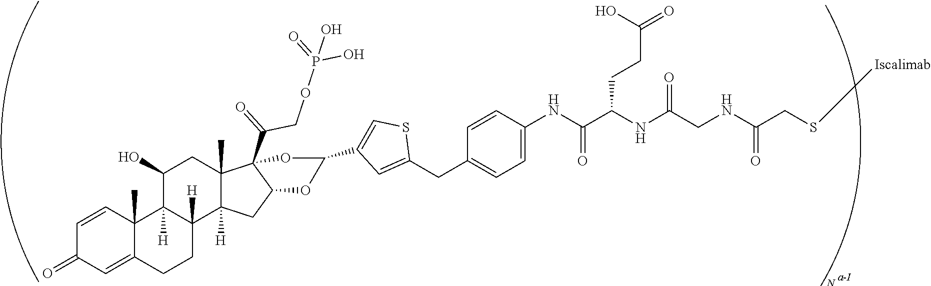 |
| II-C-612 | 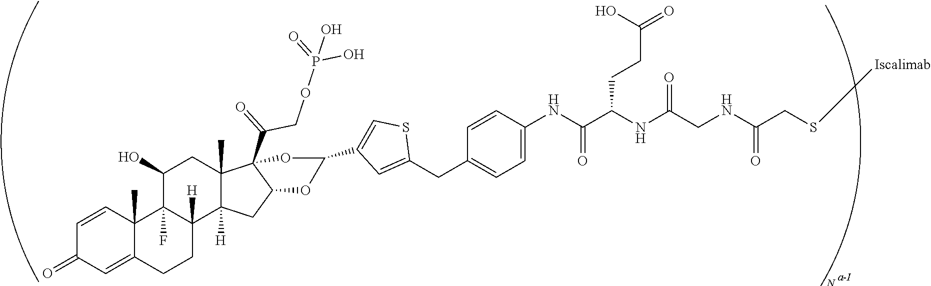 |
| II-C-617 | 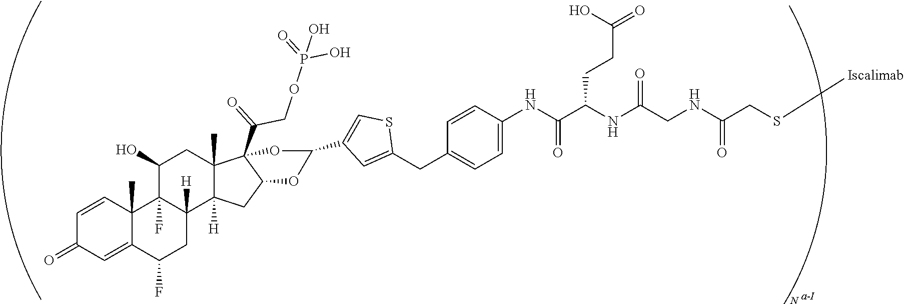 |
| II-C-622 | 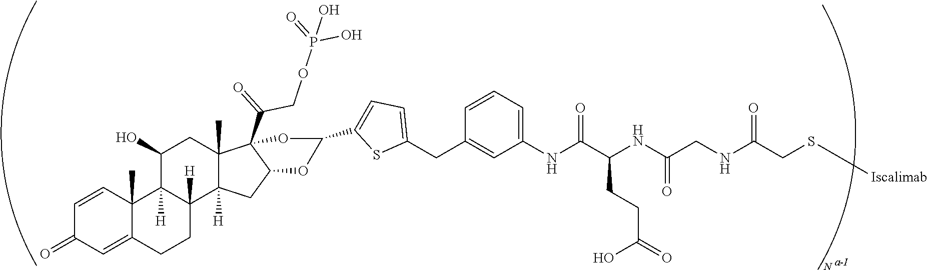 |
| II-C-627 | 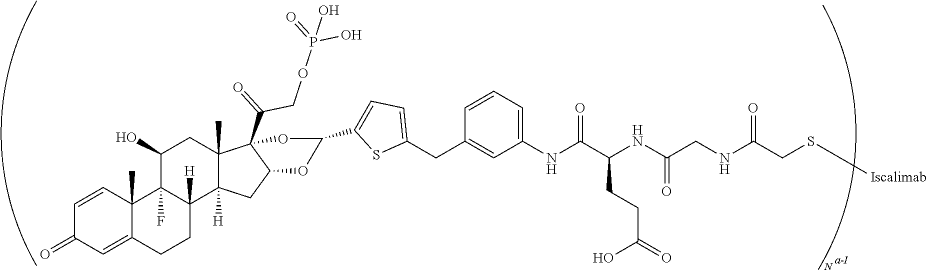 |
| II-C-632 | 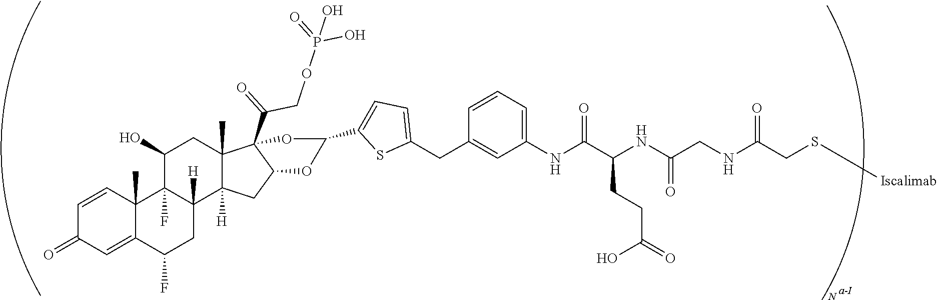 |
| II-C-637 | 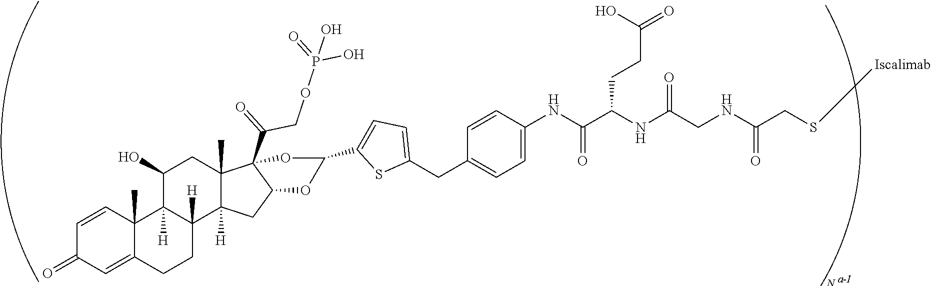 |
| II-C-642 | 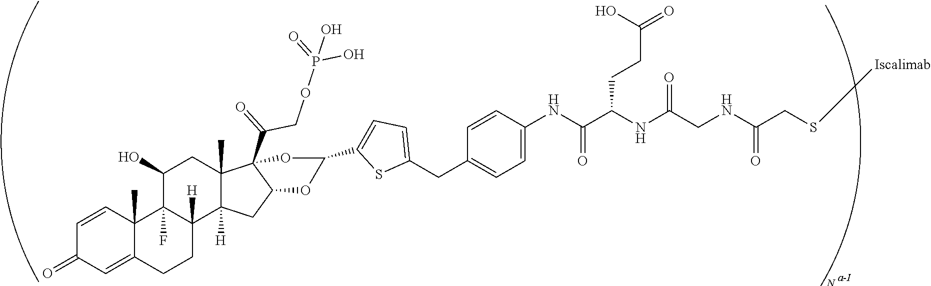 |
| II-C-647 | 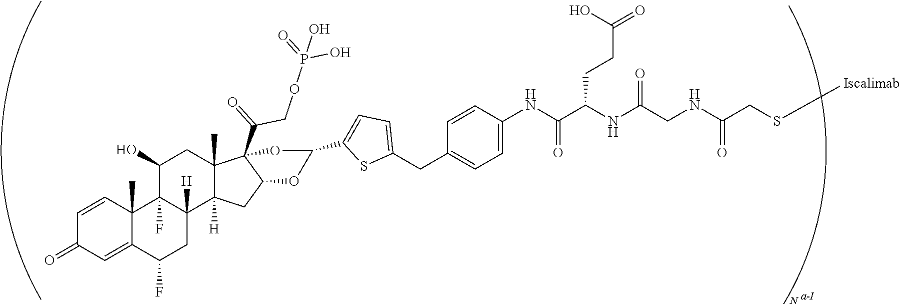 |
| II-C-659 | 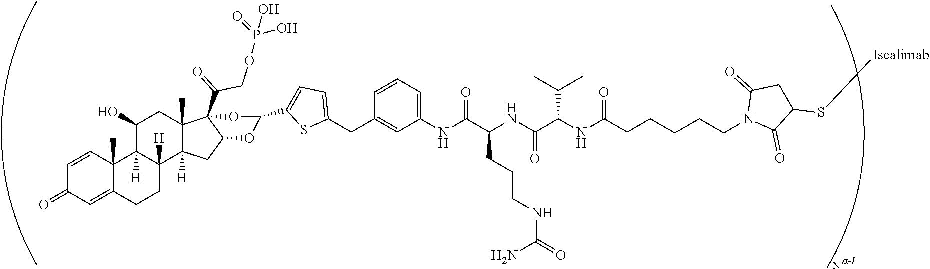 |
| II-C-661 | 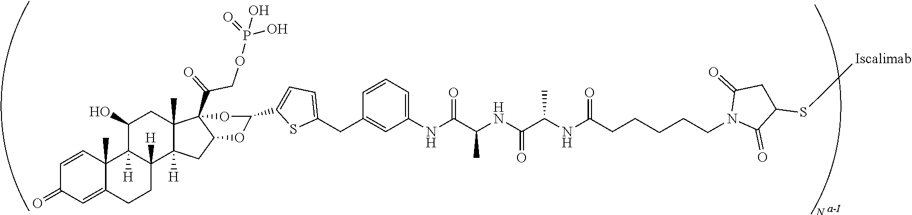 |
| II-C-663 | 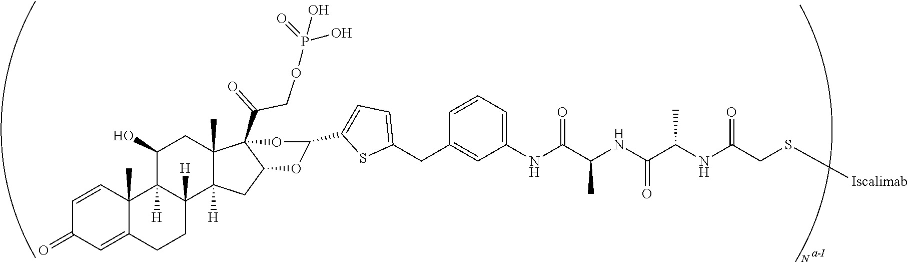 |
| II-C-712 | 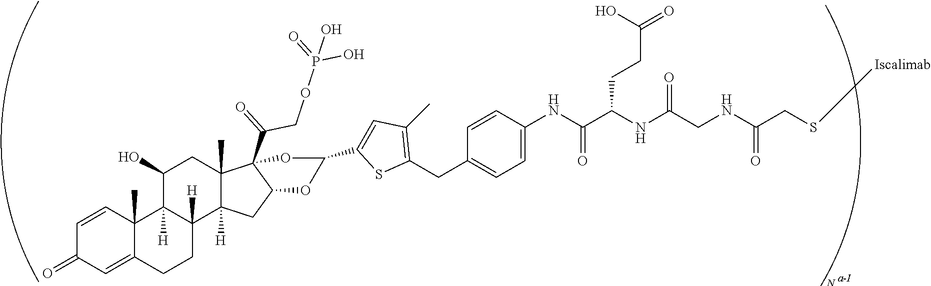 |
| II-C-715 | 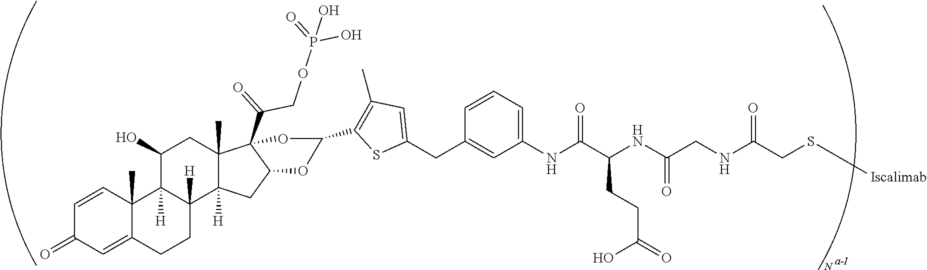 |
| II-C-724 | 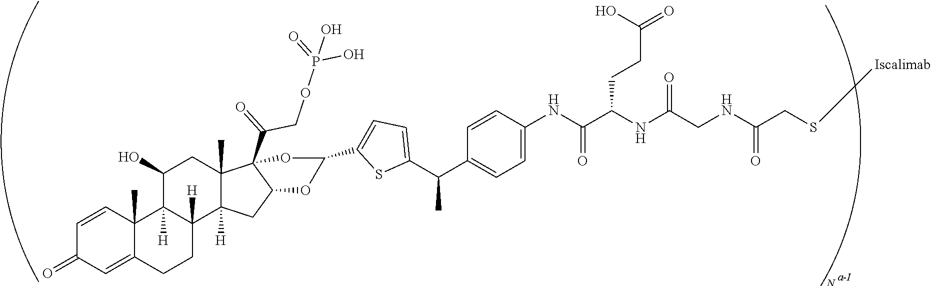 |
| II-C-727 | 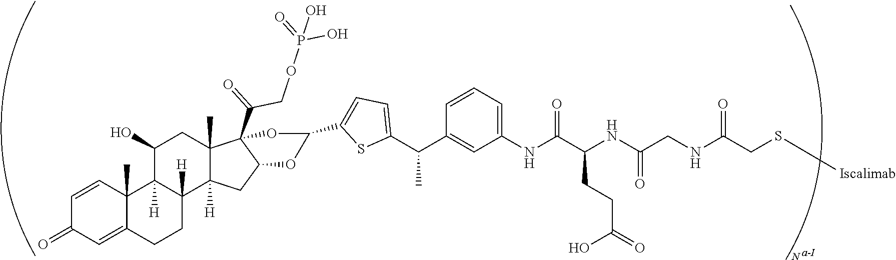 |
| II-C-734 | 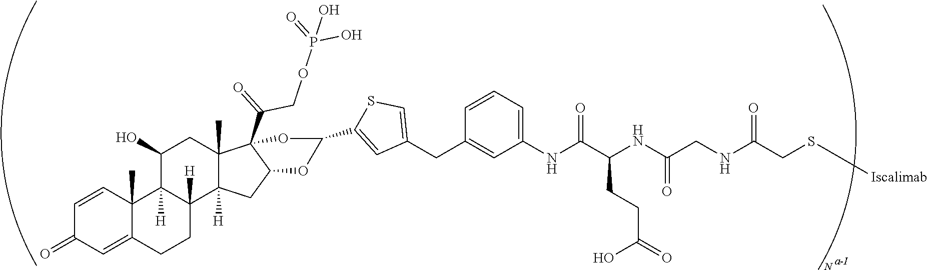 |
| II-C-739 | 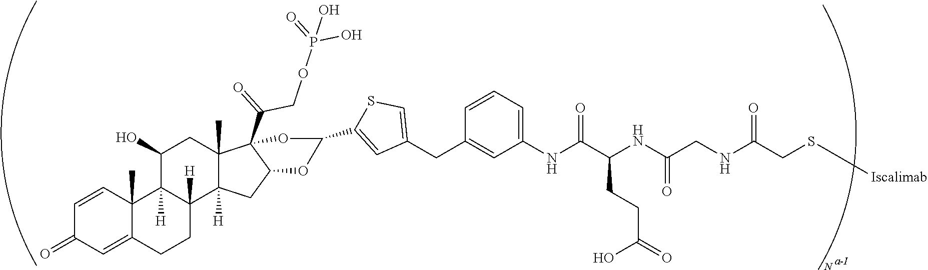 |
| II-C-744 | 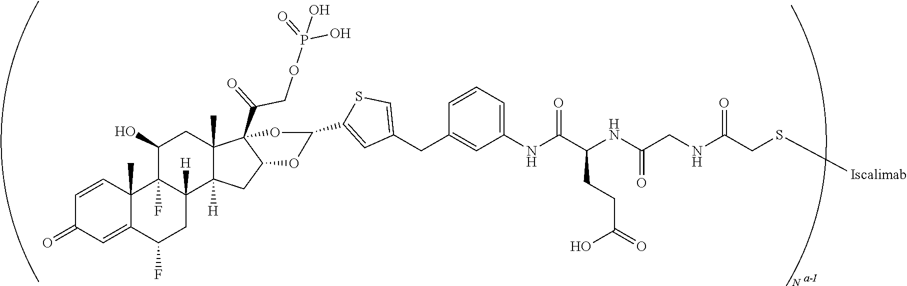 |
| II-C-749 | 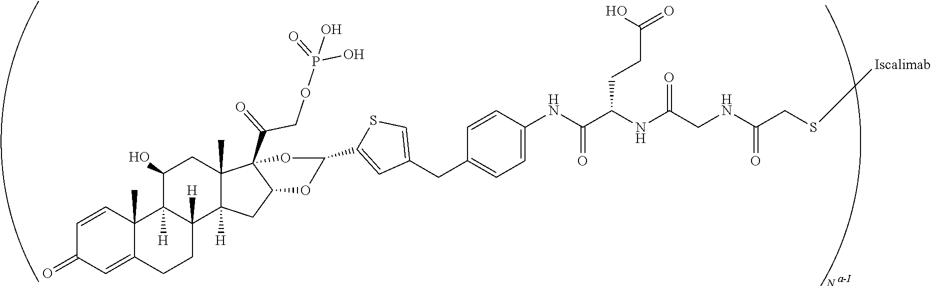 |
| II-C-754 | 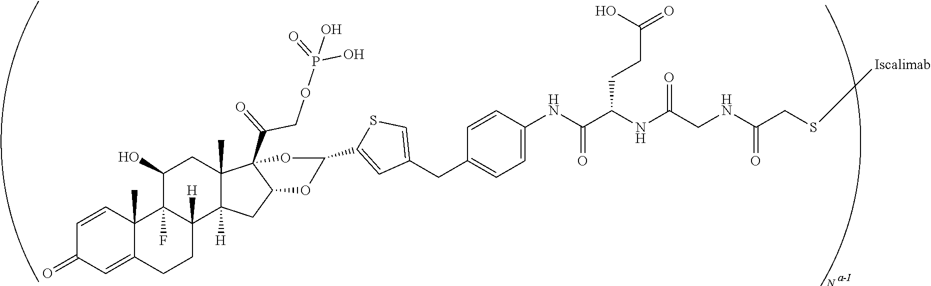 |
| II-C-758 | 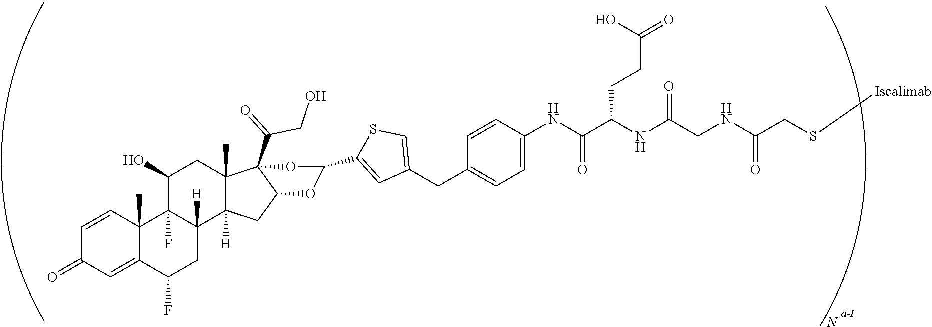 |
| II-C-759 | 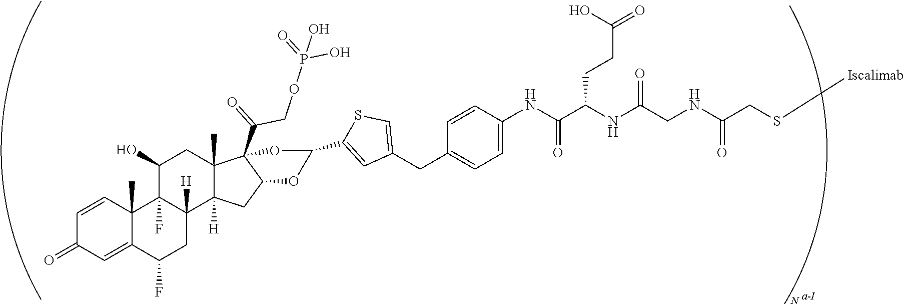 |
| II-C-763 | 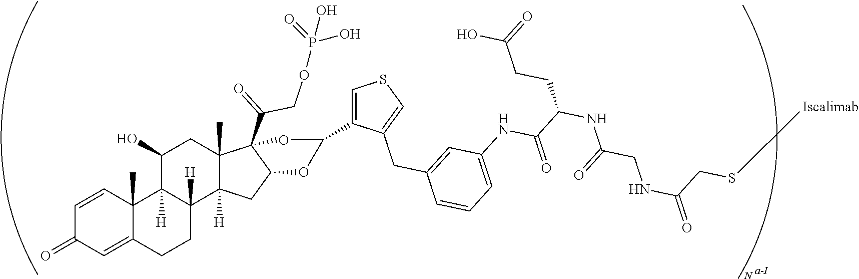 |
| II-C-764 | 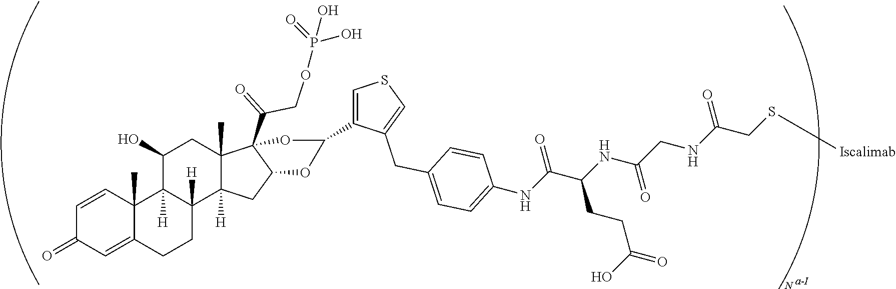 |
| II-C-765 | 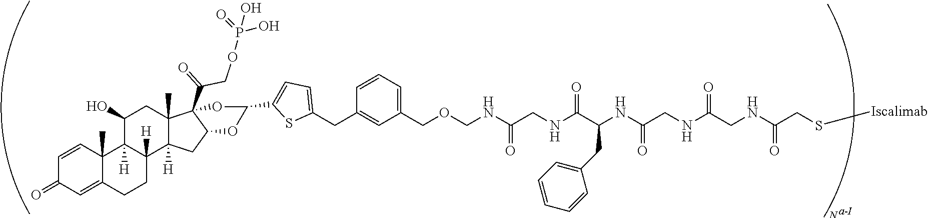 |
| II-C-766 | 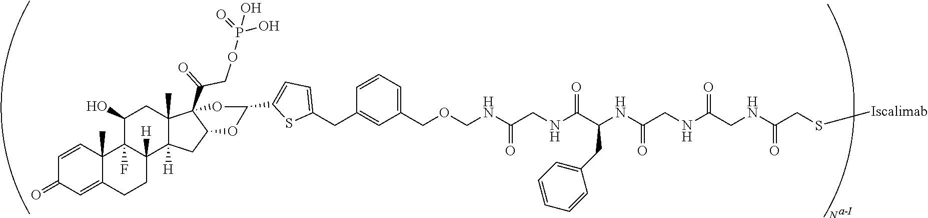 |
| II-C-767 | 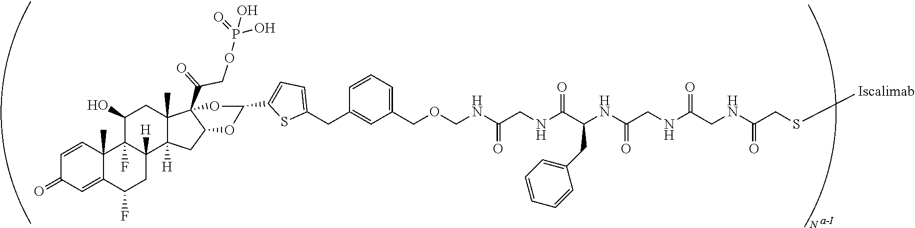 |
| II-C-768 | 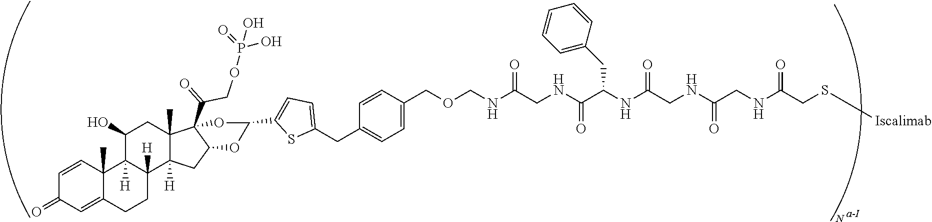 |
| II-C-769 | 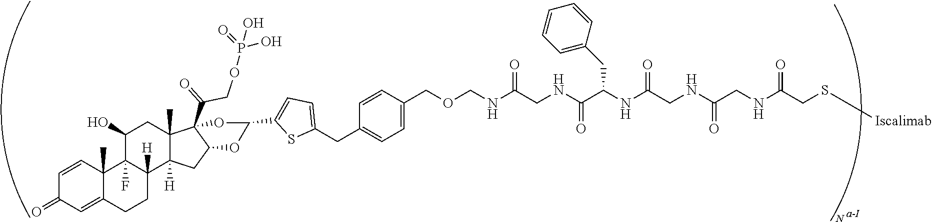 |
| II-C-770 | 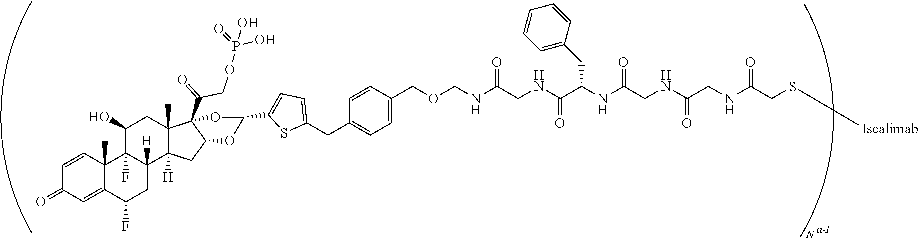 |
| II-C-771 | 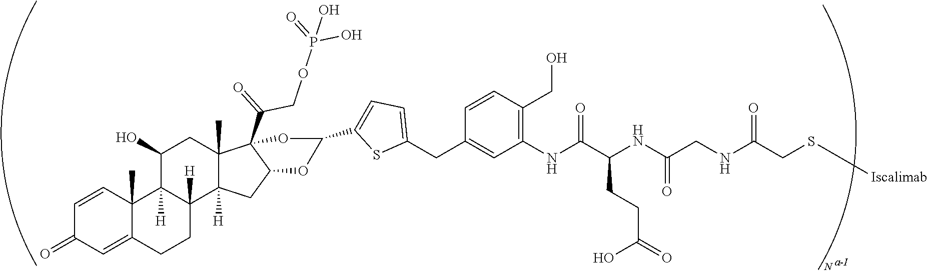 |
| II-C-774 | 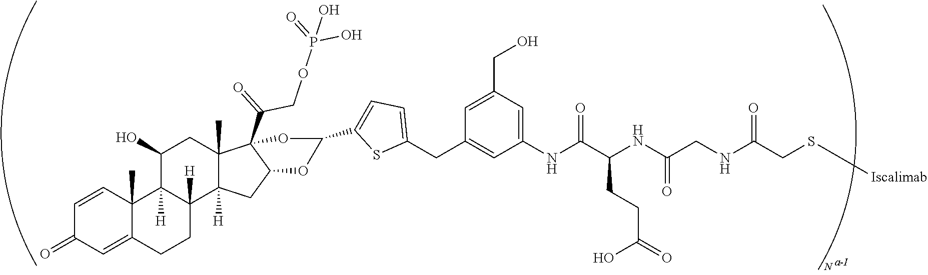 |
| II-C-777 | 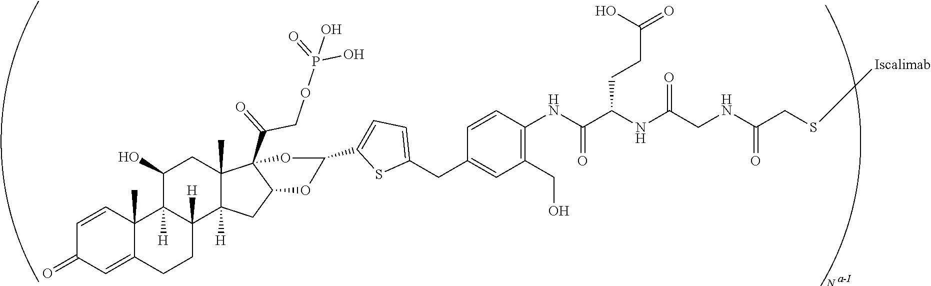 |
| II-C-780 | 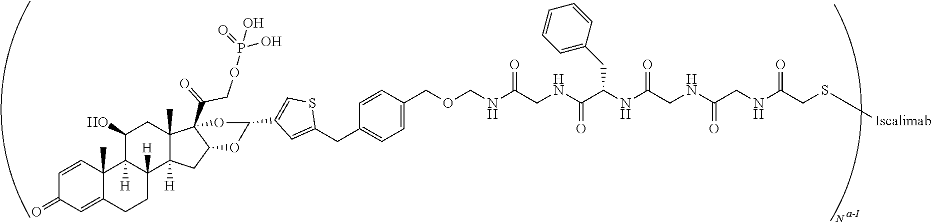 |
| II-C-781 | 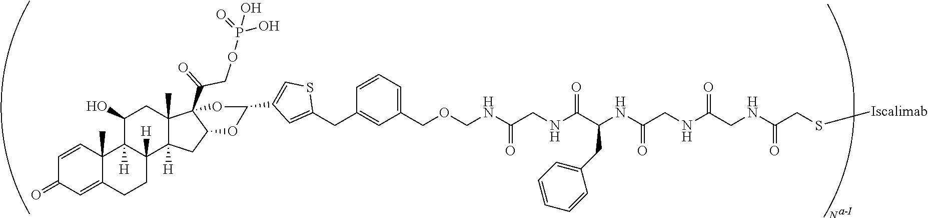 |
| II-C-782 | 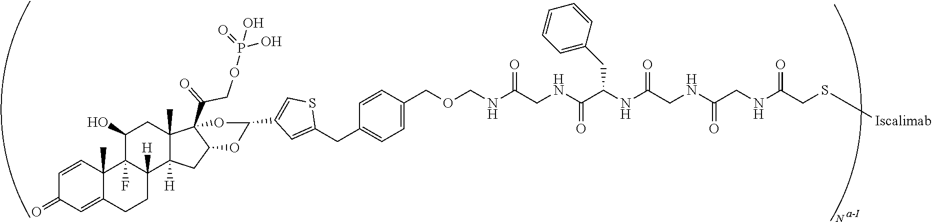 |
| II-C-783 | 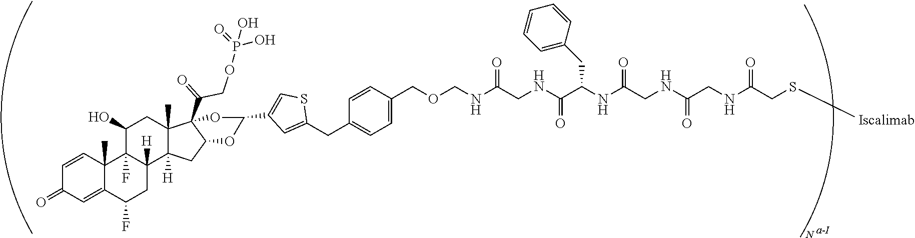 |
| II-C-784 | 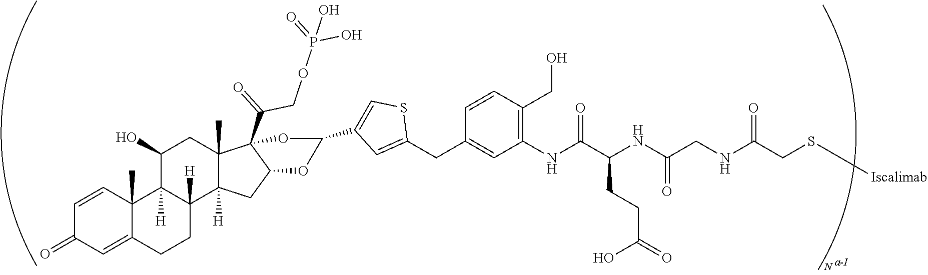 |
| II-C-787 | 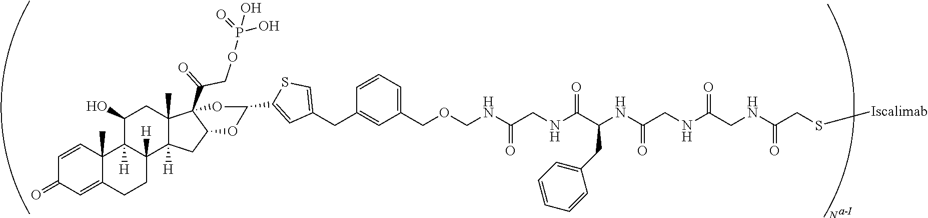 |
| II-C-788 | 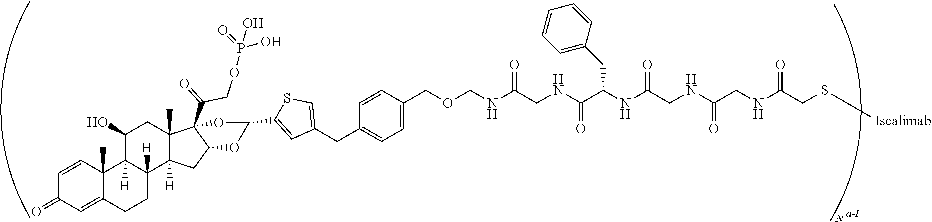 |
| II-C-789 | 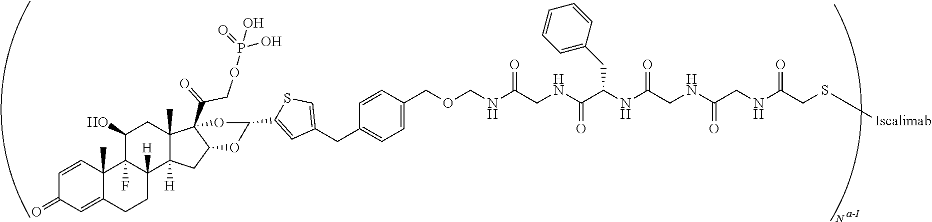 |
| II-C-790 | 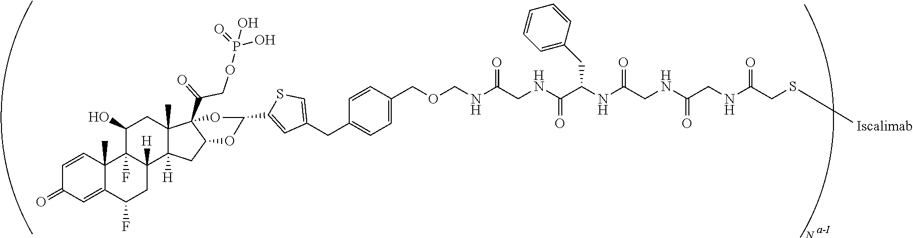 |
| II-C-791 | 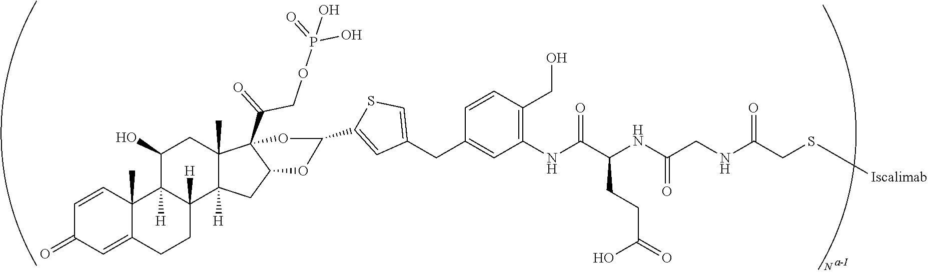 |
| II-C-794 | 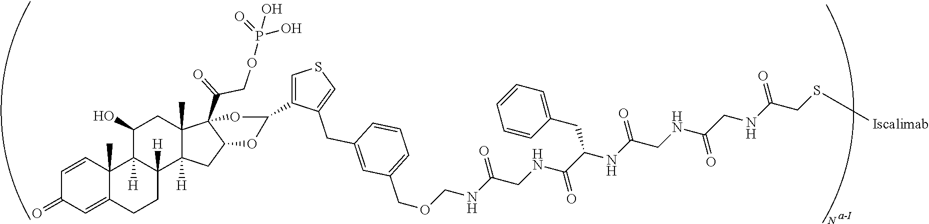 |
| II-C-795 | 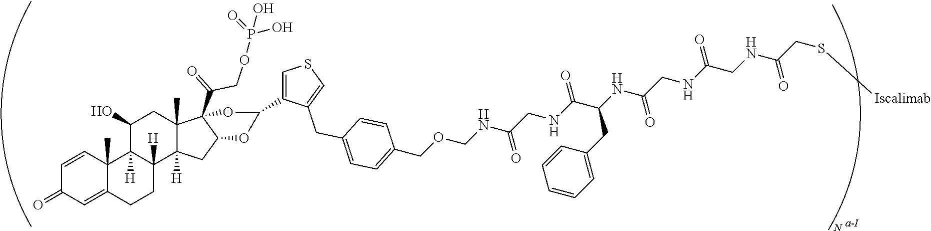 |
| II-C-814 | 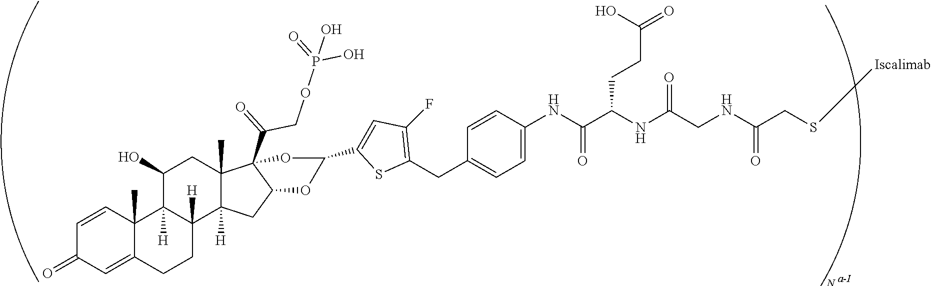 |
| II-C-817 | 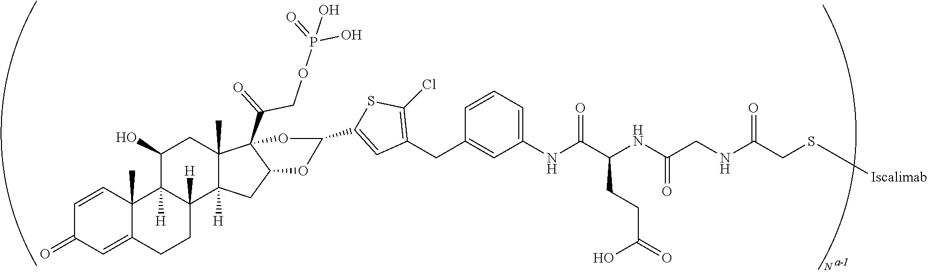 |
| II-C-826 | 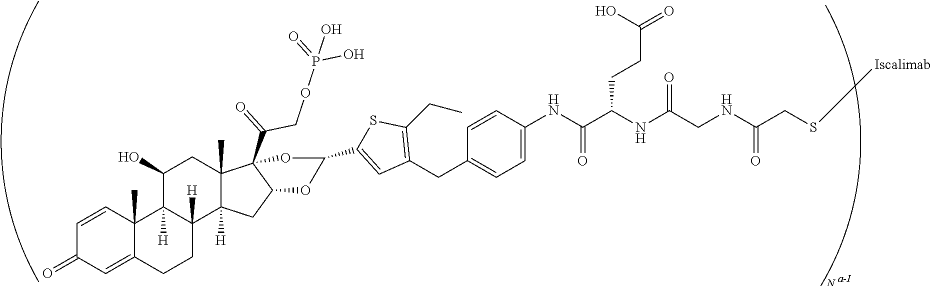 |
| II-C-829 | 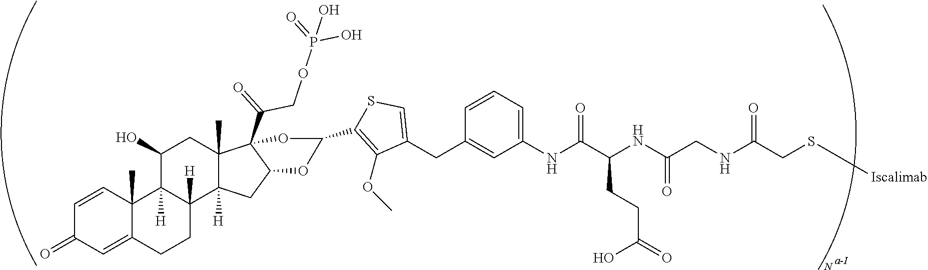 |
| II-C-838 | 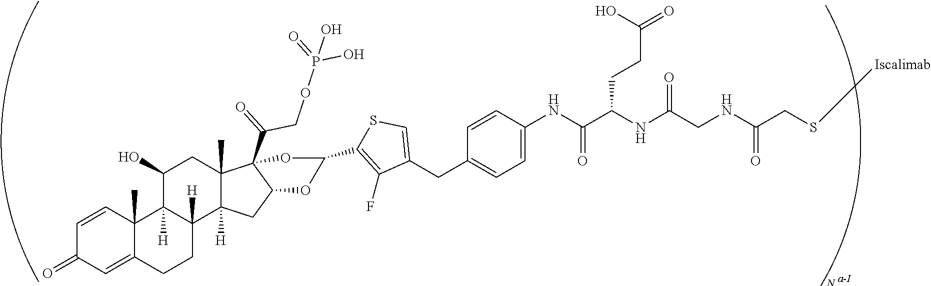 |
| II-C-841 | 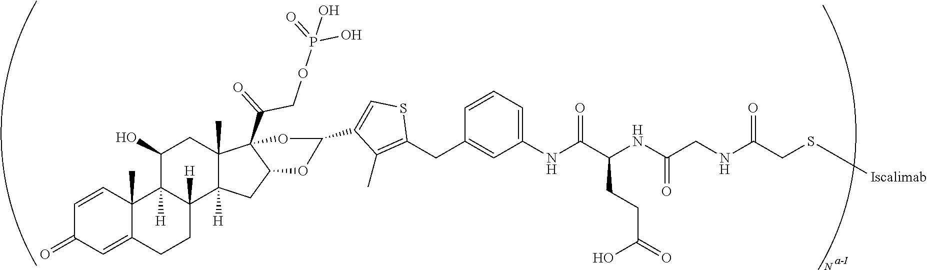 |
| II-C-850 | 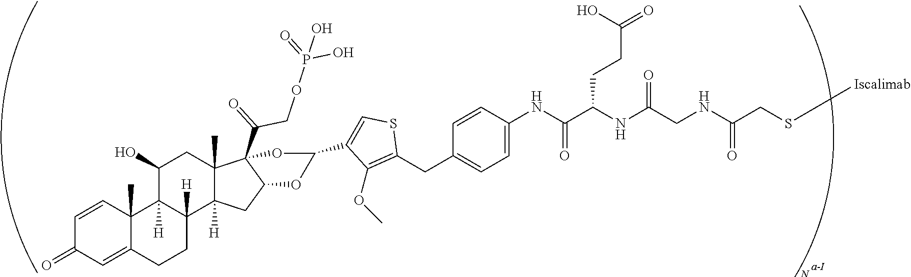 |
| II-C-853 | 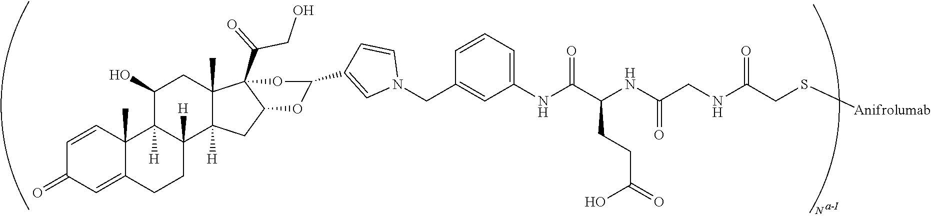 |
| II-C-854 | 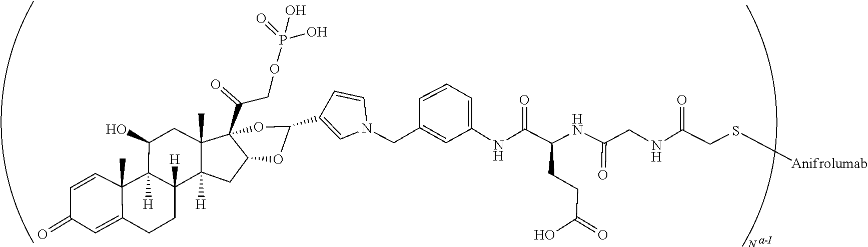 |
| II-C-859 | 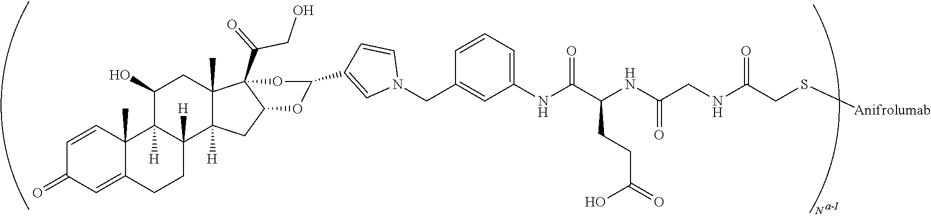 |
| II-C-860 | 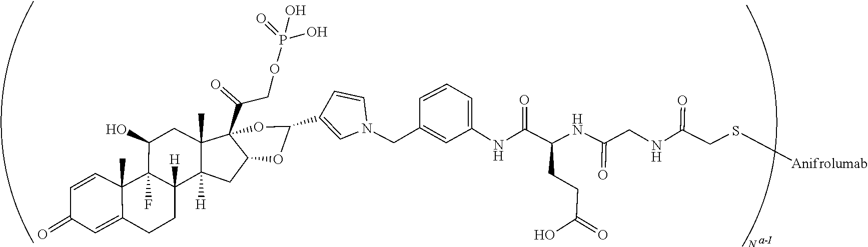 |
| II-C-864 | 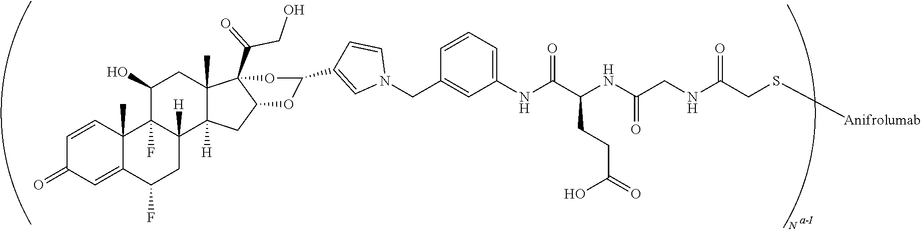 |
| II-C-865 | 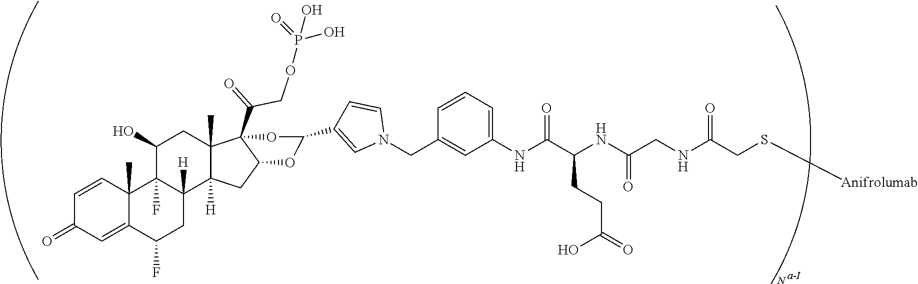 |
| II-C-869 | 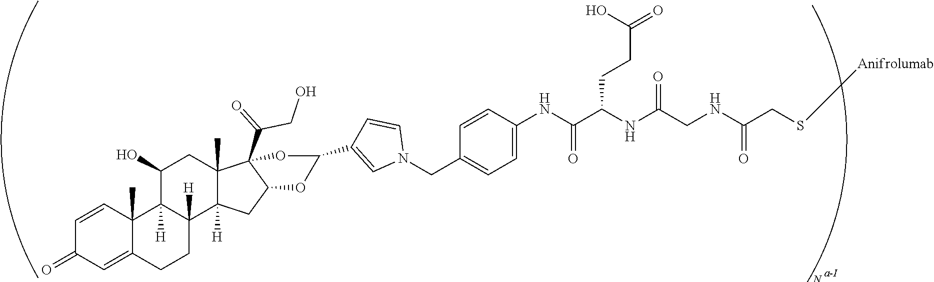 |
| II-C-870 | 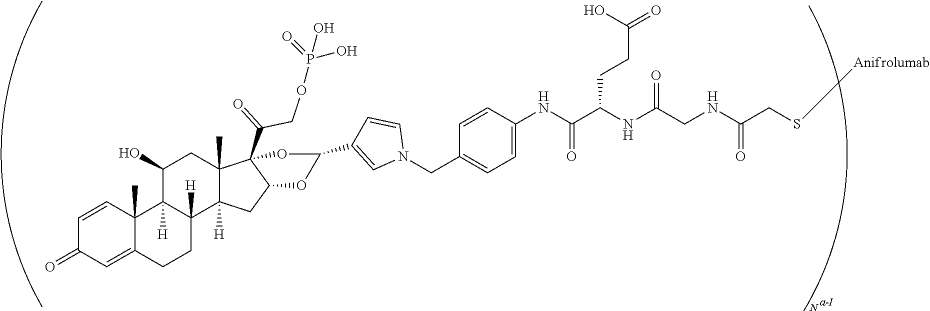 |
| II-C-875 | 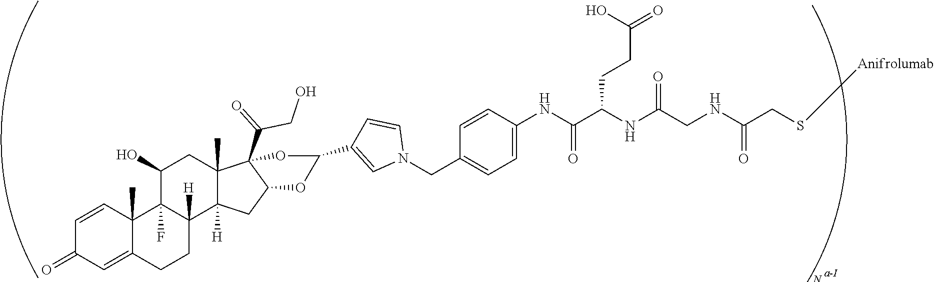 |
| II-C-876 | 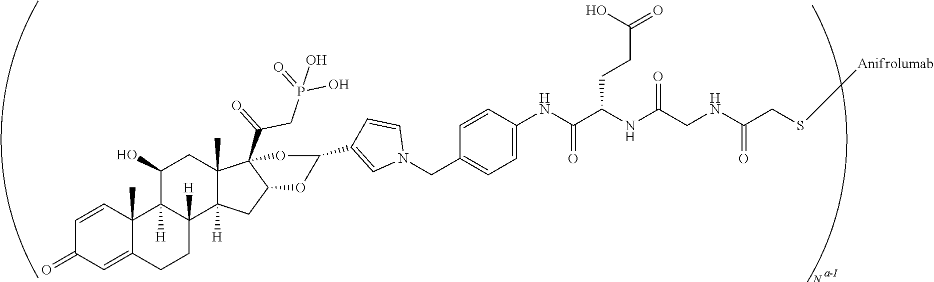 |
| II-C-881 | 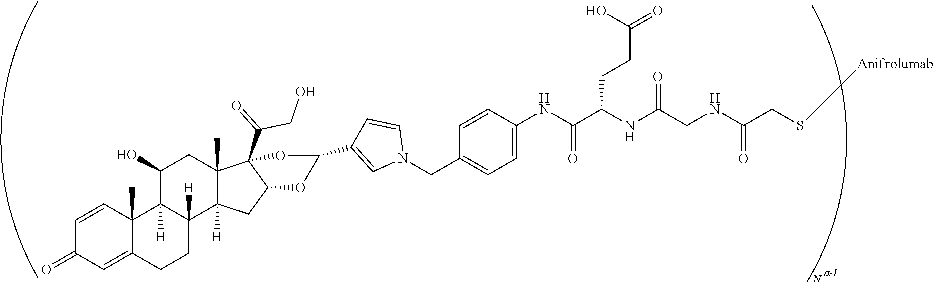 |
| II-C-882 | 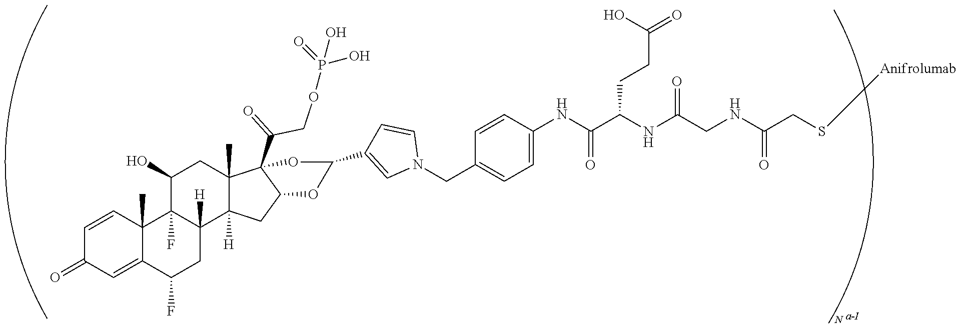 |
| II-C-886 | 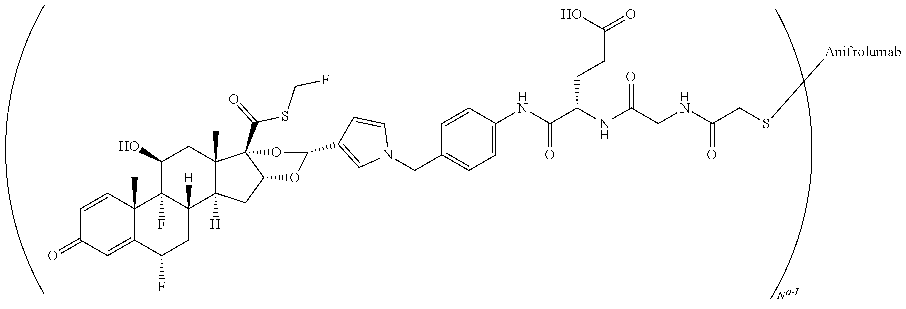 |
| II-C-887 | 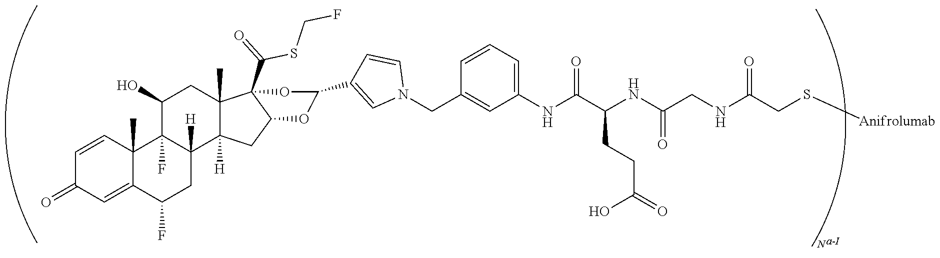 |
| II-C-963 | 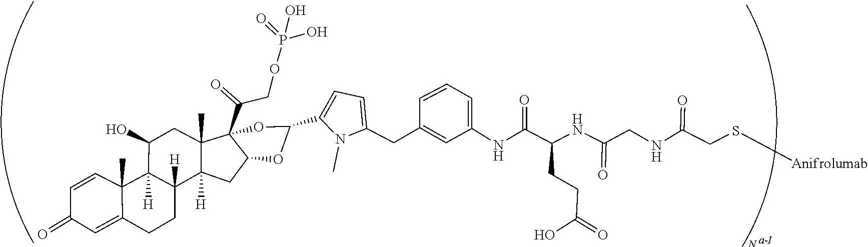 |
| II-C-966 | 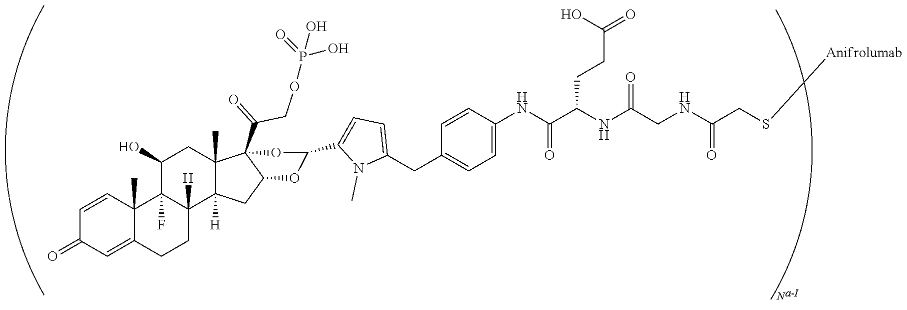 |
| II-C-969 | 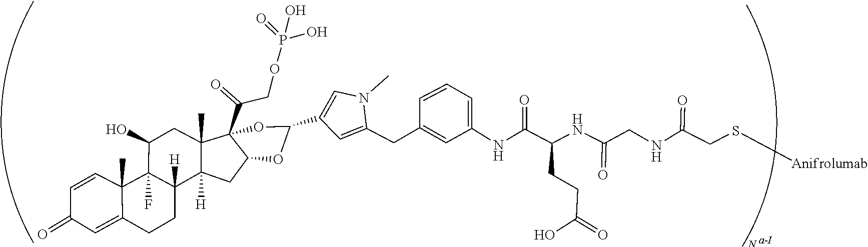 |
| II-C-972 | 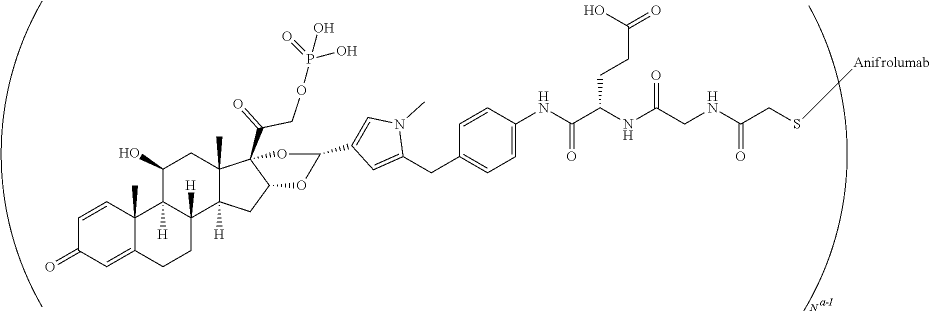 |
| II-C-975 | 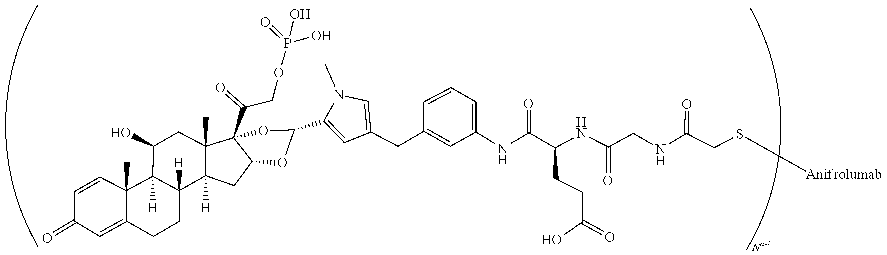 |
| II-C-978 | 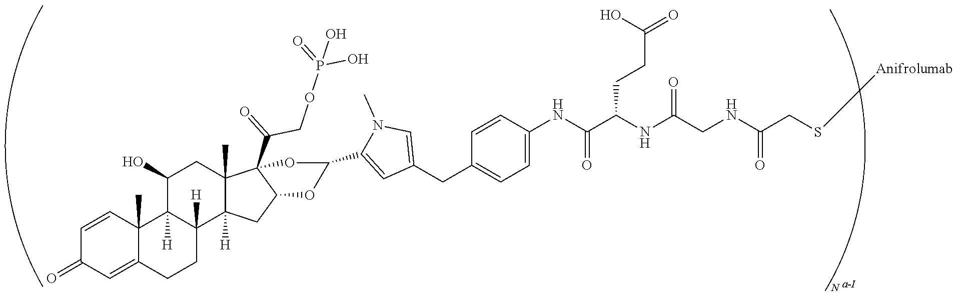 |
| II-C- 1017 | 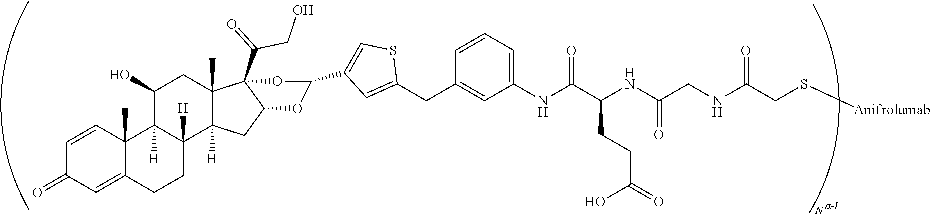 |
| II-C- 1018 | 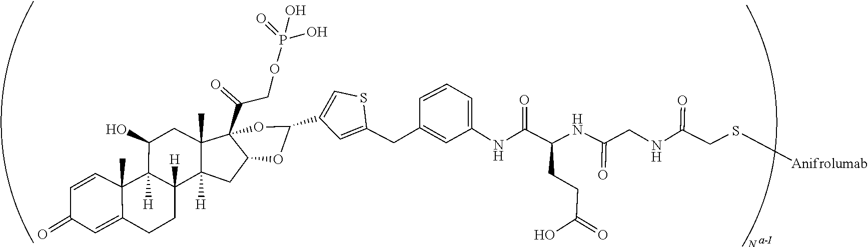 |
| II-C- 1022 | 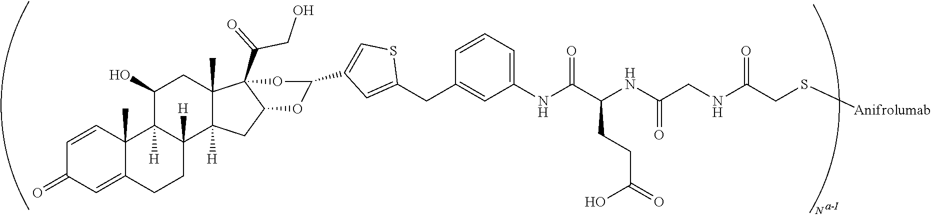 |
| II-C- 1023 | 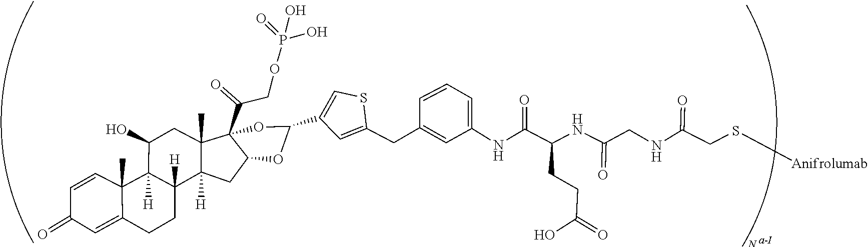 |
| II-C- 1027 | 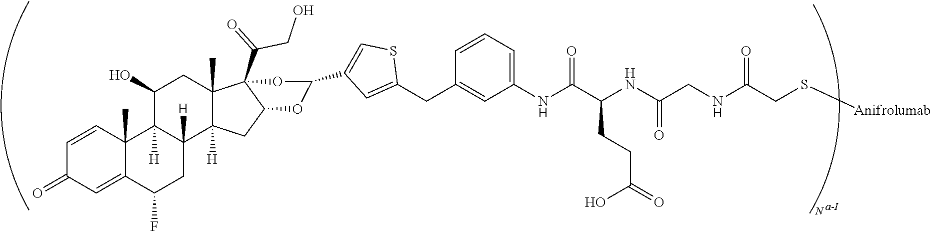 |
| II-C- 1028 | 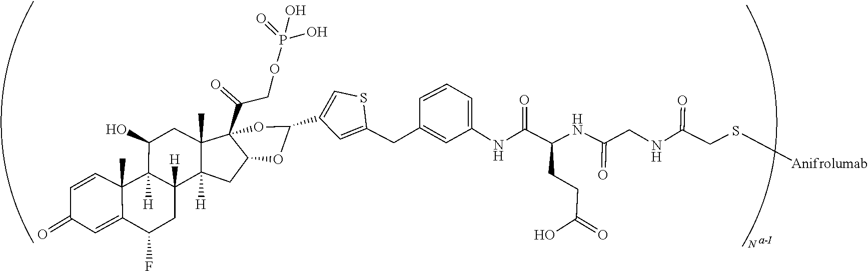 |
| II-C- 1032 | 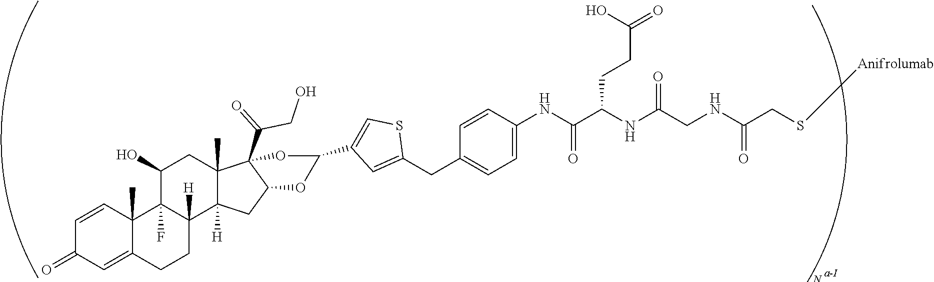 |
| II-C- 1033 | 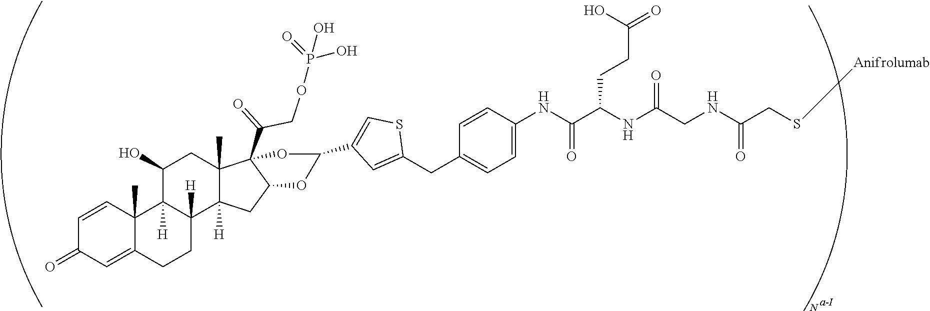 |
| II-C- 1037 | 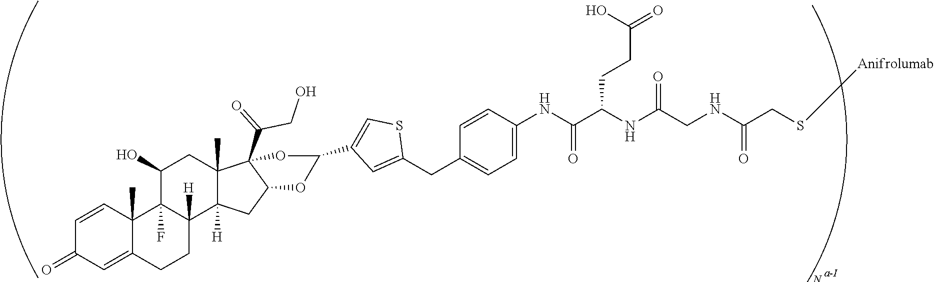 |
| II-C- 1038 | 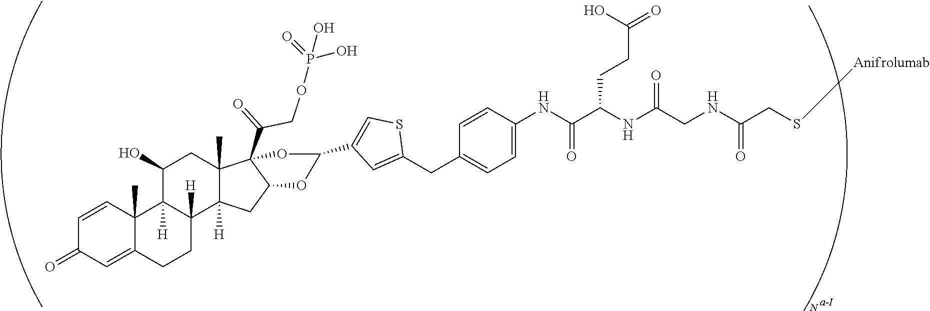 |
| II-C- 1042 | 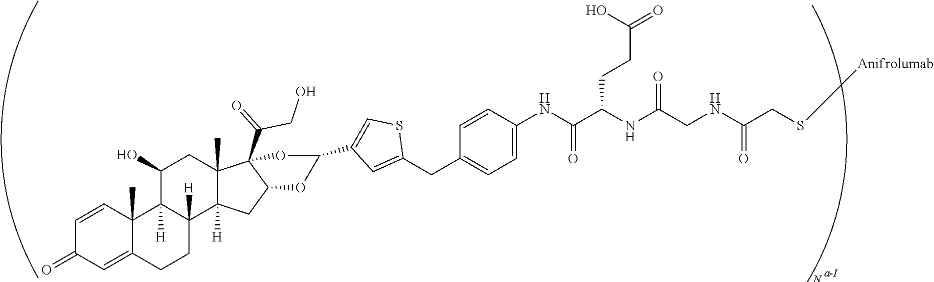 |
| II-C- 1043 | 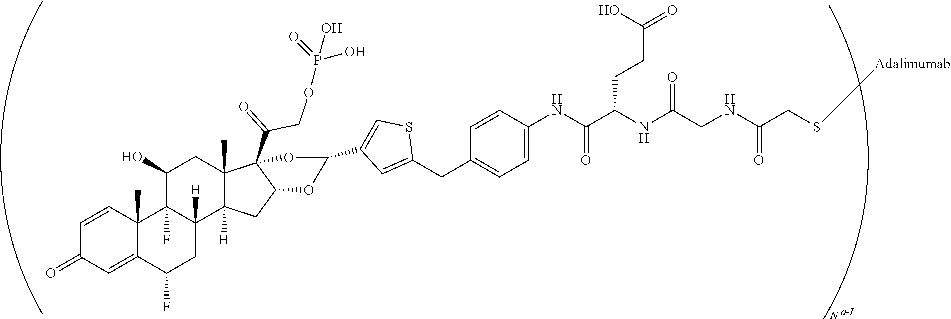 |
| II-C- 1047 | 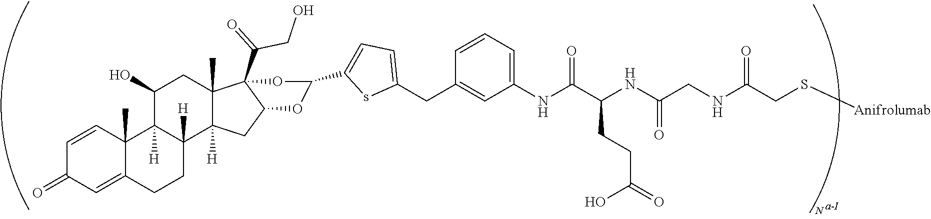 |
| II-C- 1048 | 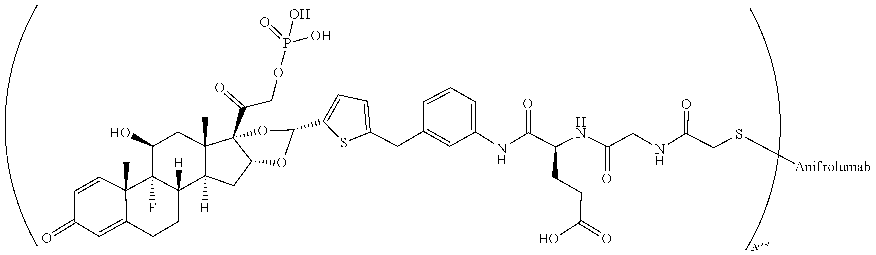 |
| II-C- 1052 | 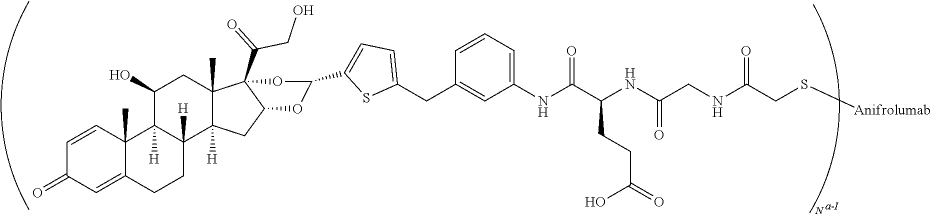 |
| II-C- 1053 | 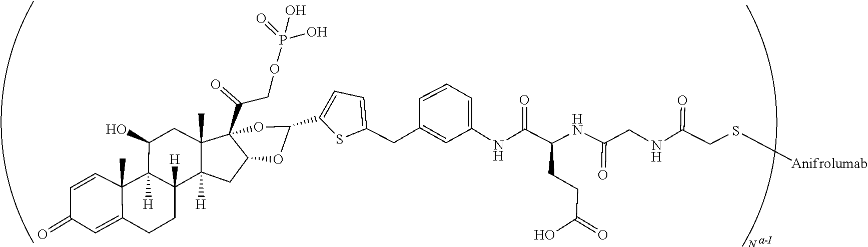 |
| II-C- 1057 | 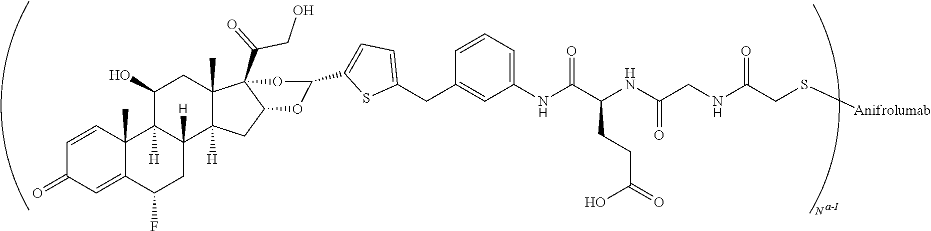 |
| II-C- 1058 | 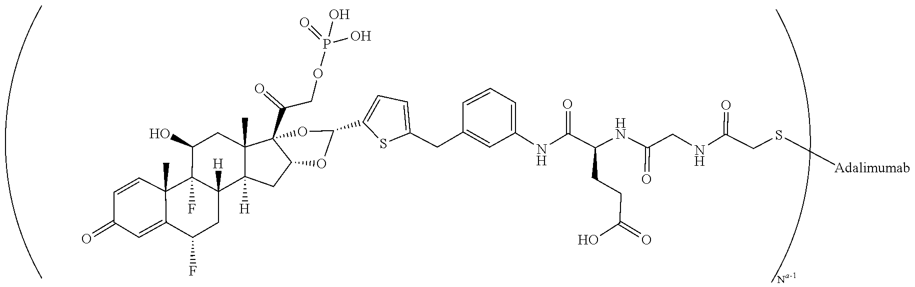 |
| II-C- 1062 | 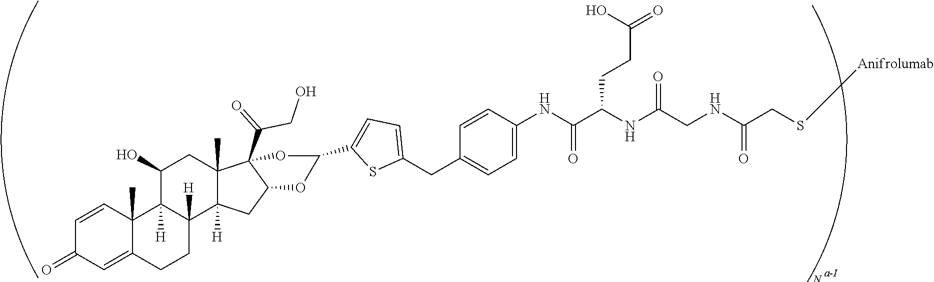 |
| II-C- 1063 | 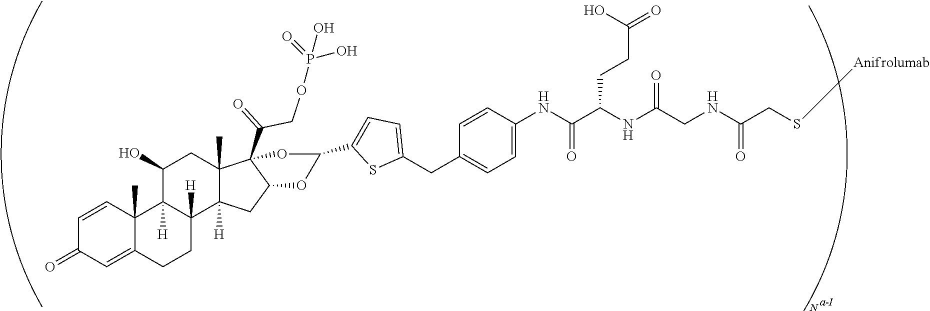 |
| II-C- 1067 | 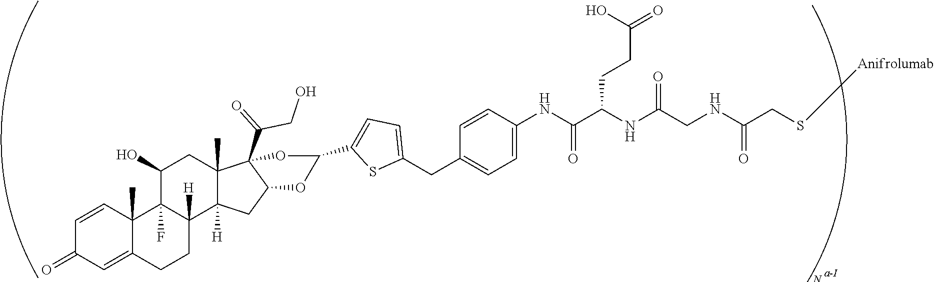 |
| II-C- 1068 | 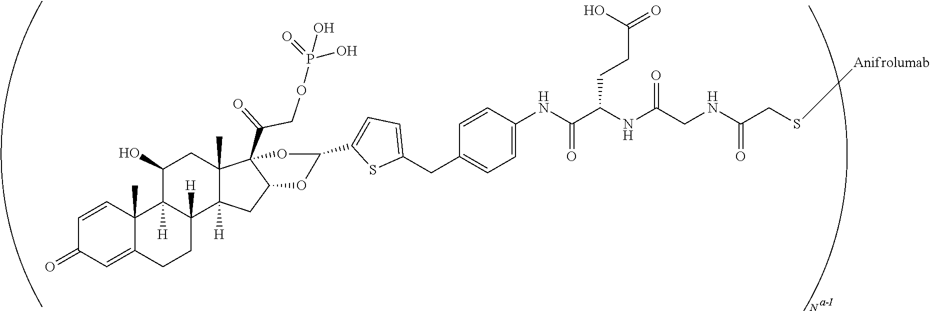 |
| II-C- 1072 | 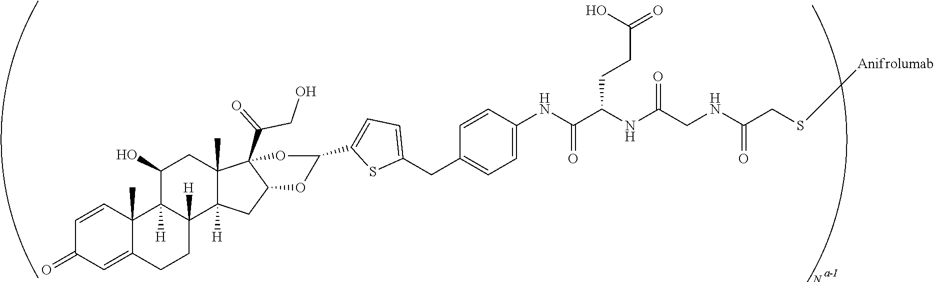 |
| II-C- 1073 | 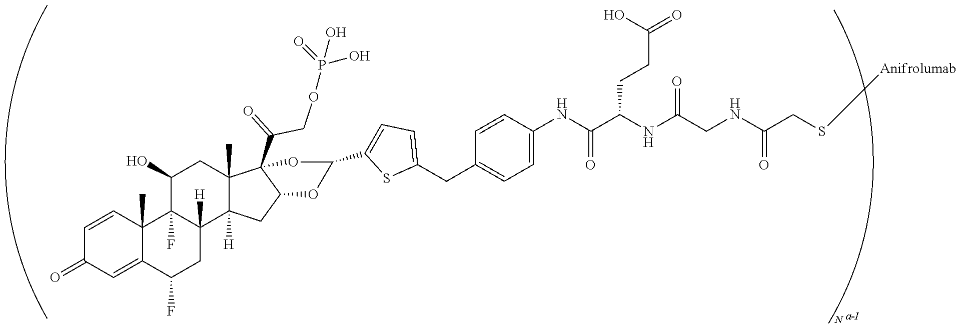 |
| II-C- 1085 | 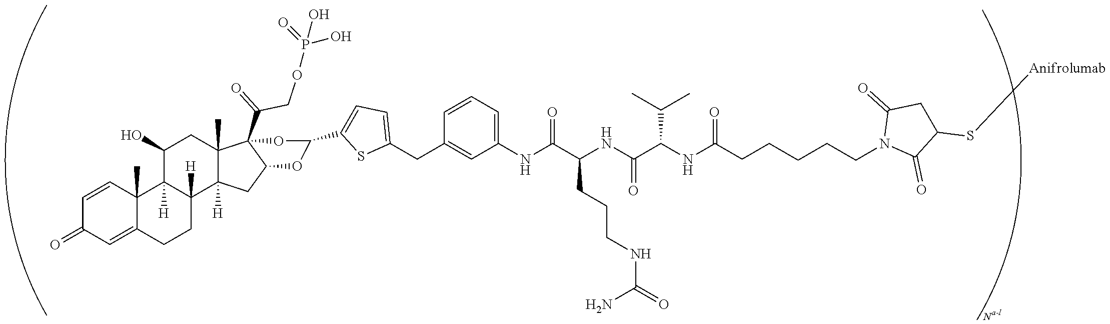 |
| II-C- 1086 | 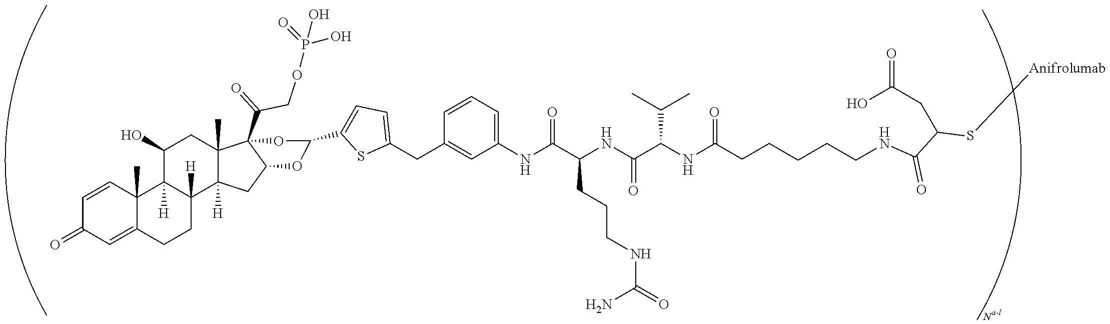 |
| II-C- 1087 | 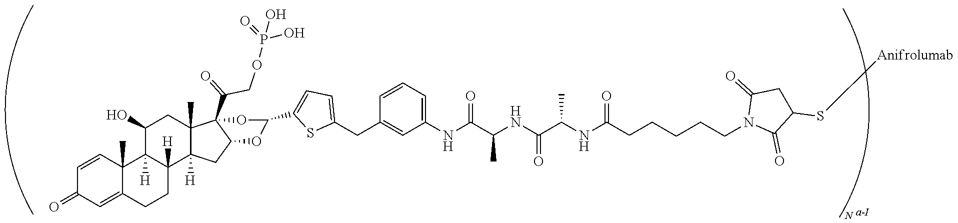 |
| II-C- 1088 | 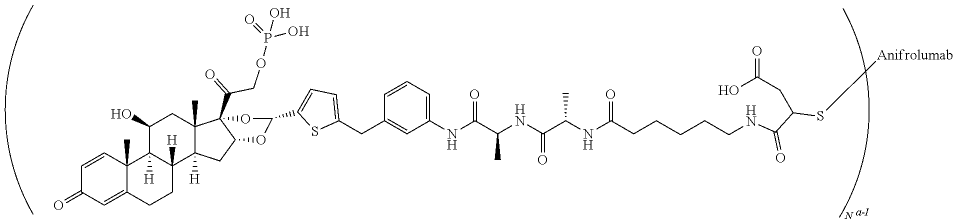 |
| II-C- 1089 | 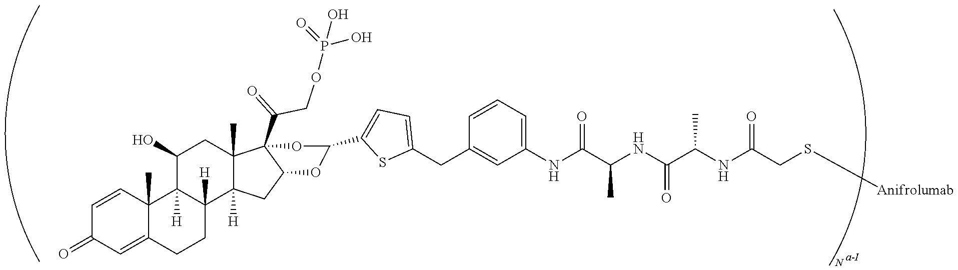 |
| II-C- 1135 | 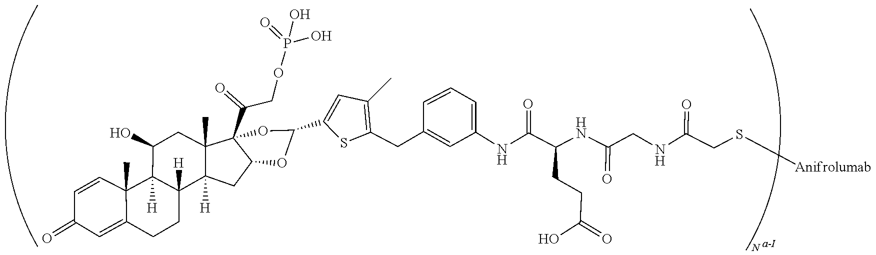 |
| II-C- 1138 | 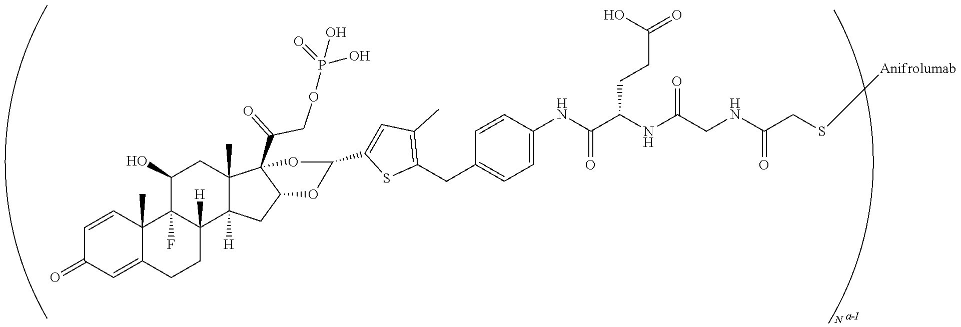 |
| II-C- 1141 | 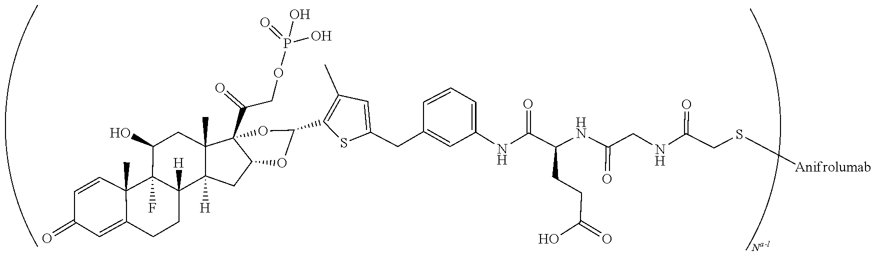 |
| II-C- 1144 | 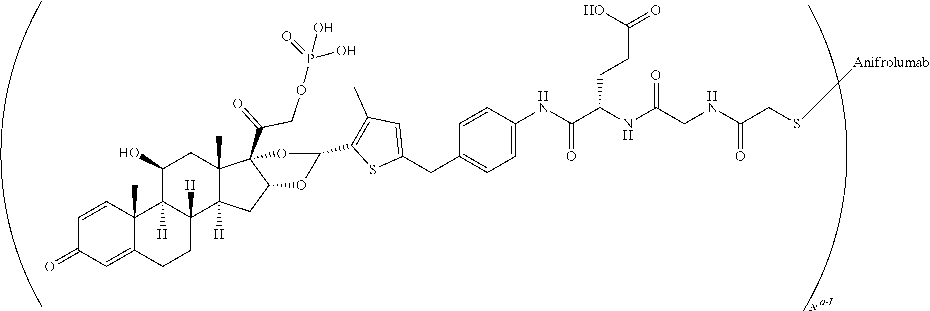 |
| II-C- 1147 | 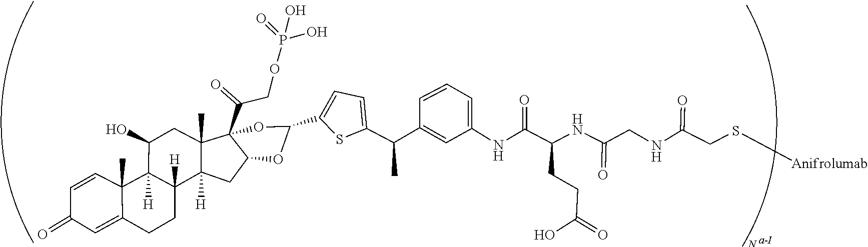 |
| II-C- 1150 | 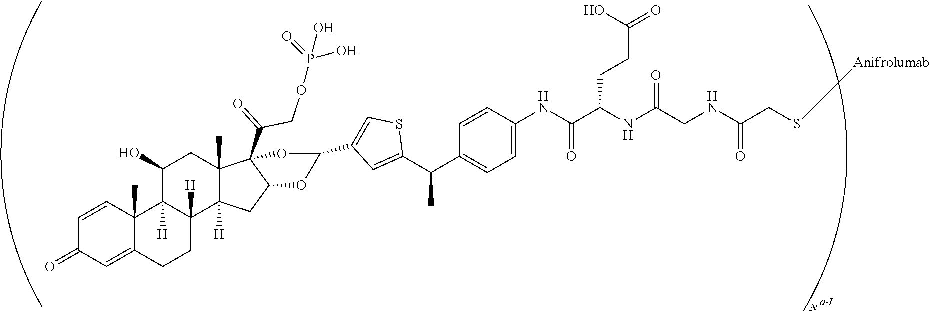 |
| II-C- 1153 | 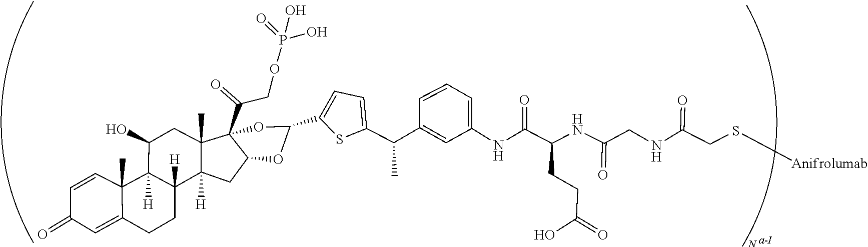 |
| II-C- 1156 | 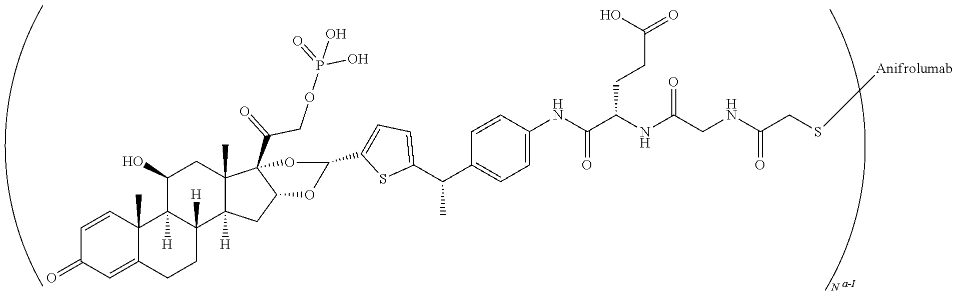 |
| II-C- 1159 | 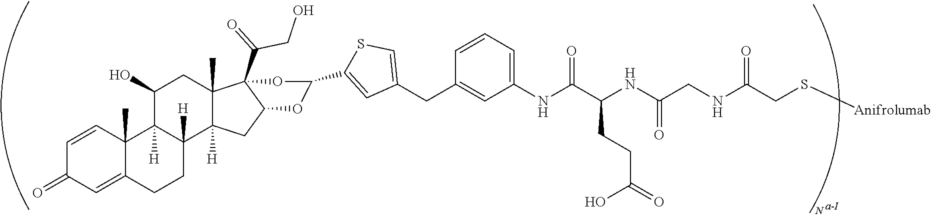 |
| II-C- 1160 | 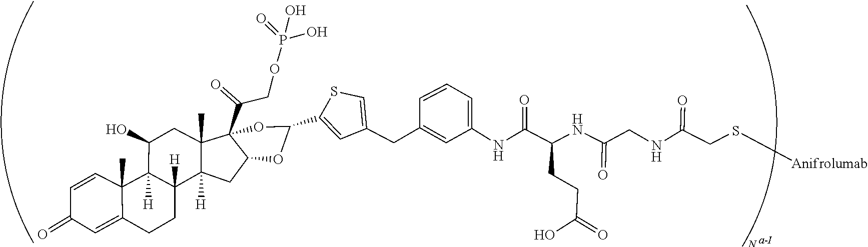 |
| II-C- 1174 | 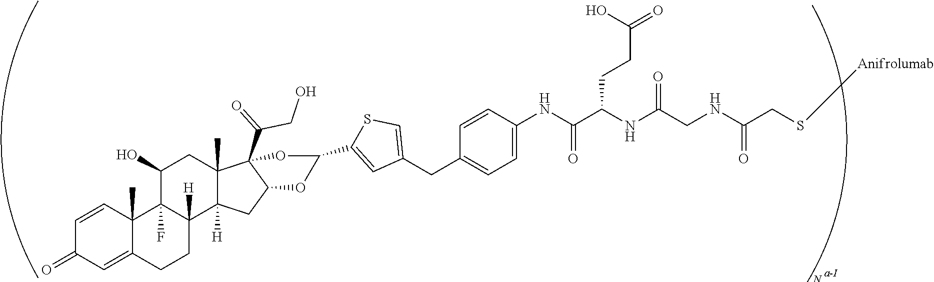 |
| II-C- 1175 | 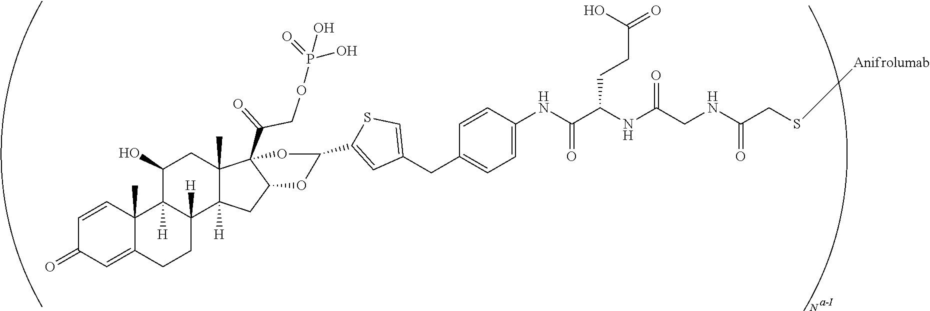 |
| II-C- 1189 | 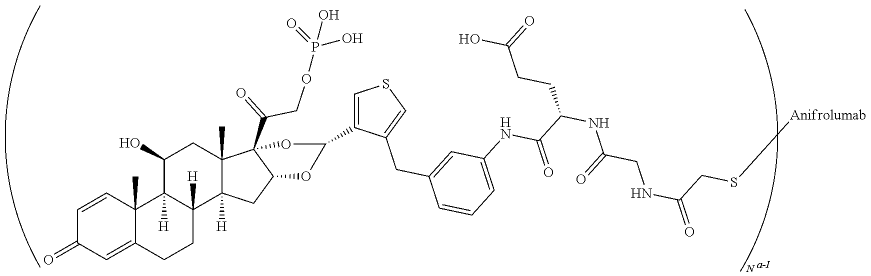 |
| II-C- 1190 | 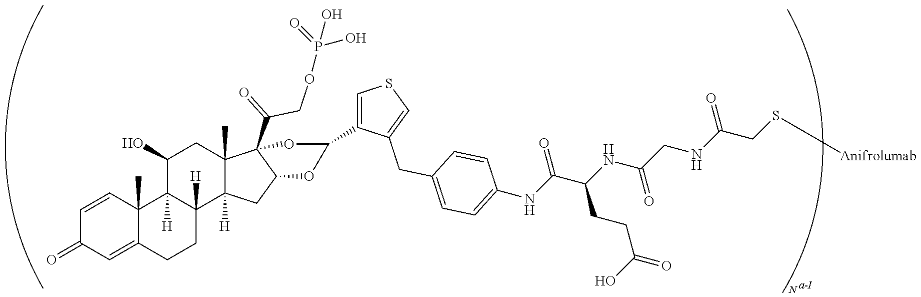 |
| II-C- 1191 | 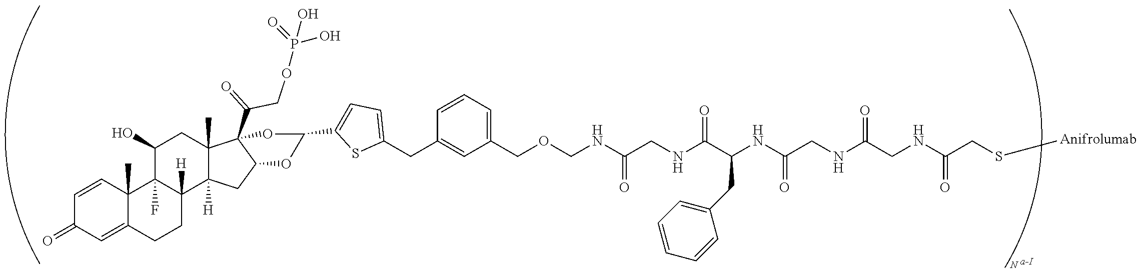 |
| II-C- 1192 | 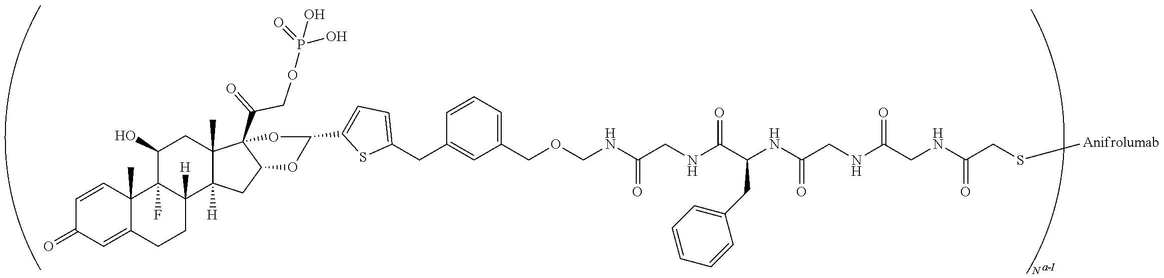 |
| II-C- 1193 | 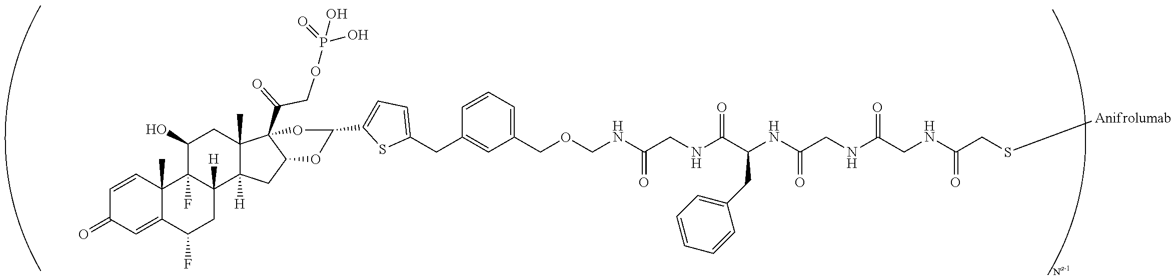 |
| II-C- 1194 | 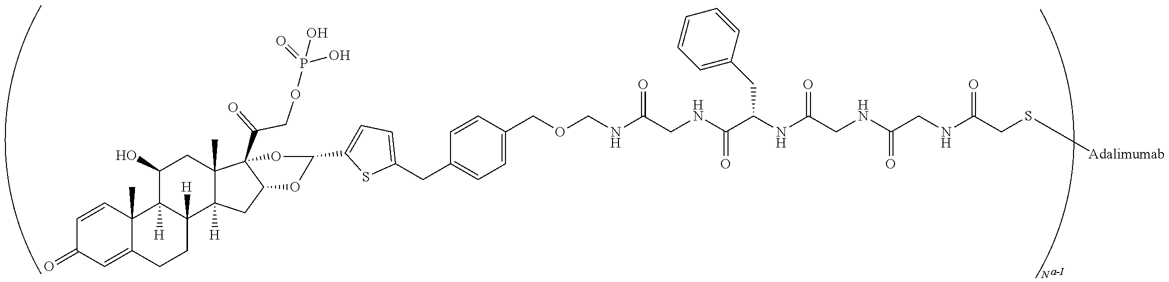 |
| II-C- 1195 | 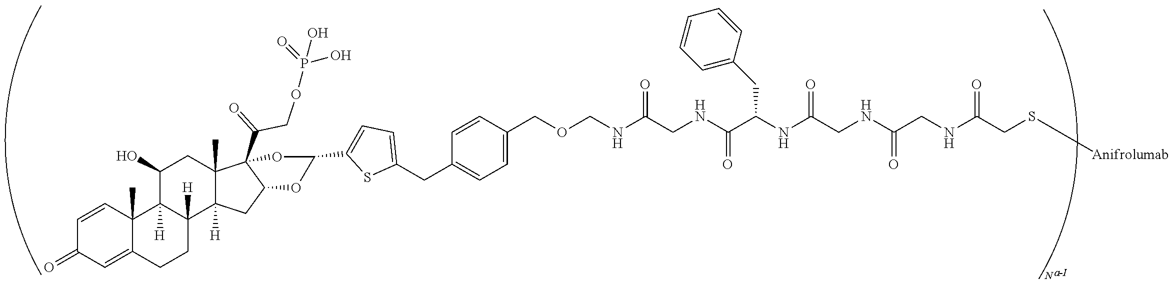 |
| II-C- 1196 | 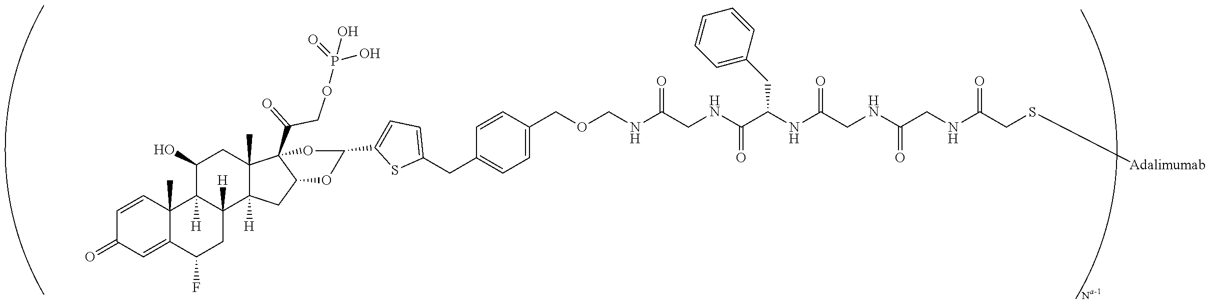 |
| II-C- 1197 | 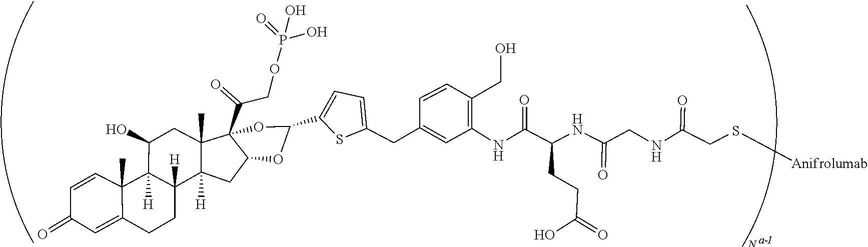 |
| II-C- 1200 | 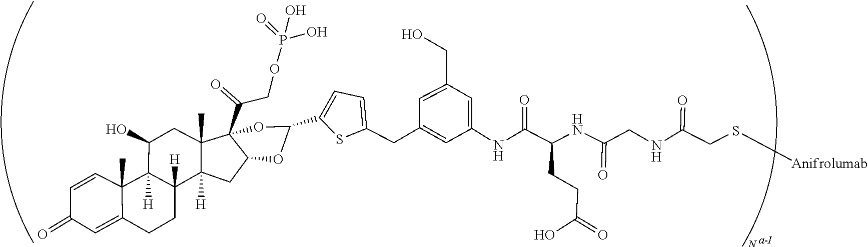 |
| II-C- 1203 | 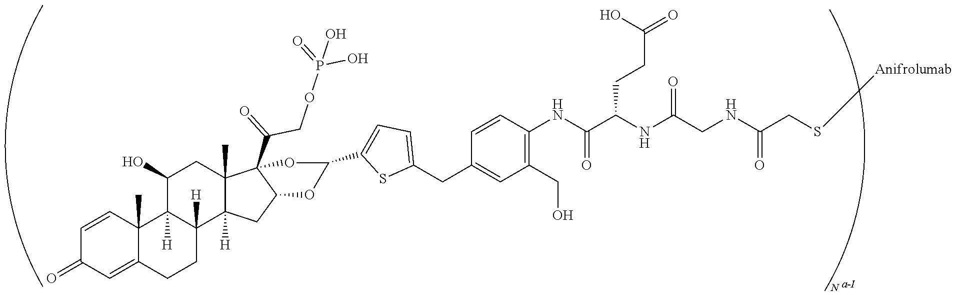 |
| II-C- 1206 | 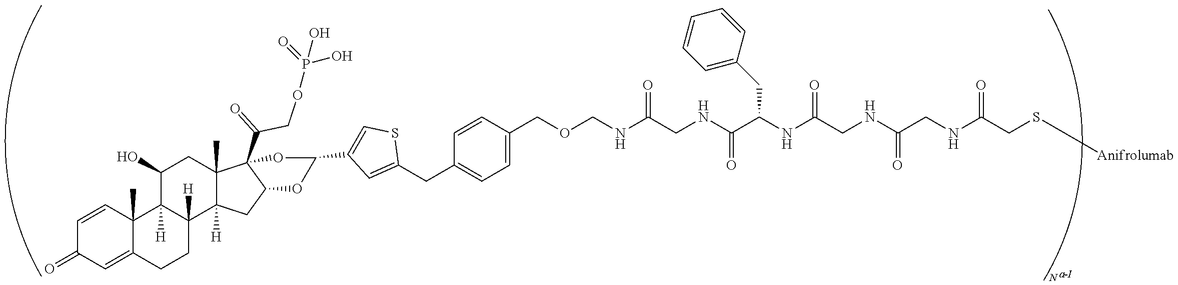 |
| II-C- 1207 | 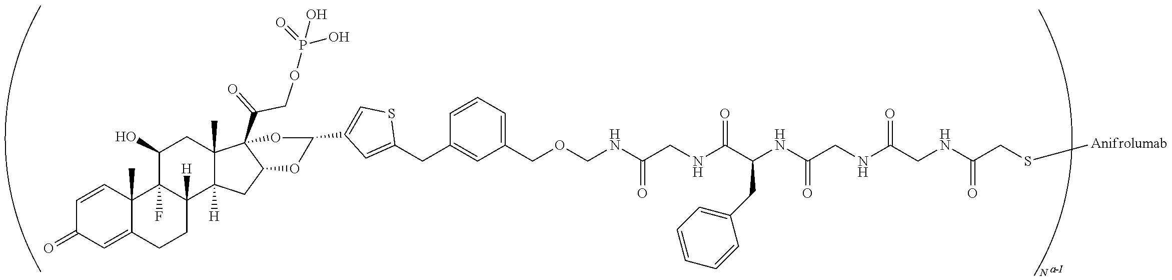 |
| II-C- 1208 | 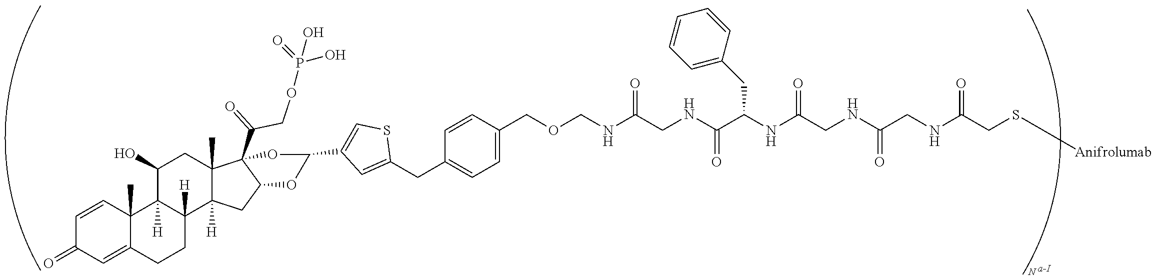 |
| II-C- 1209 | 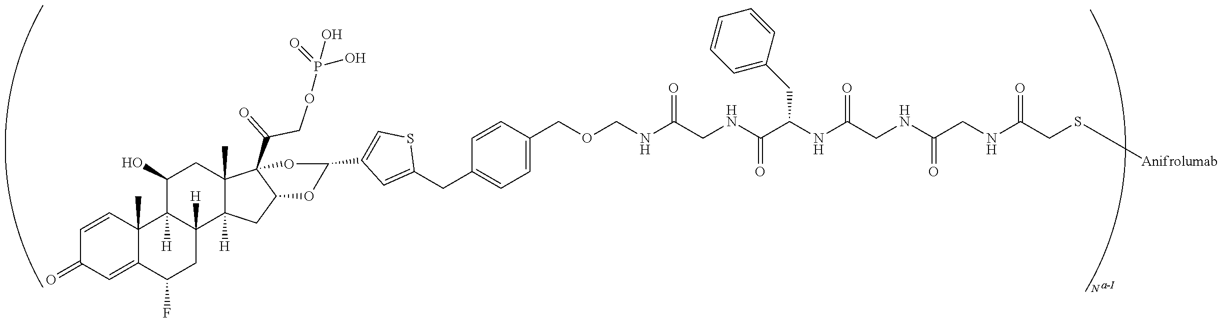 |
| II-C- 1210 | 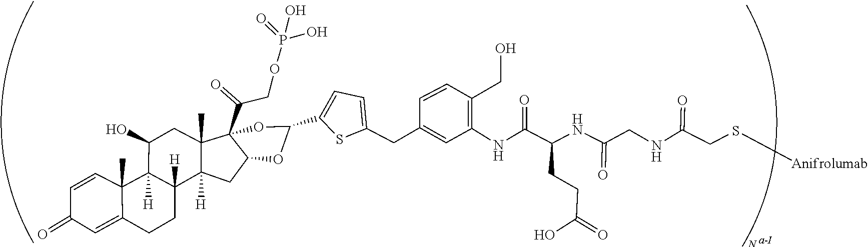 |
| II-C- 1213 | 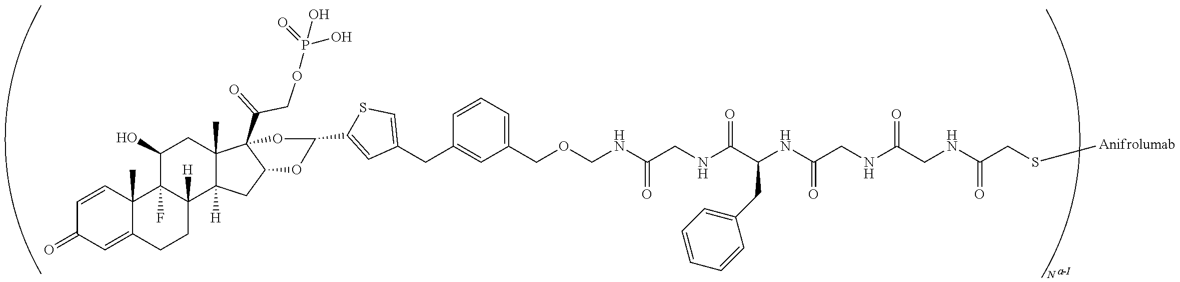 |
| II-C- 1214 | 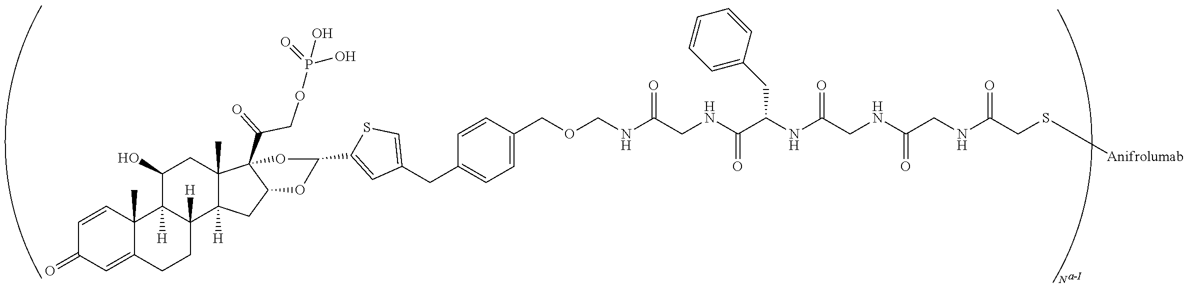 |
| II-C- 1215 | 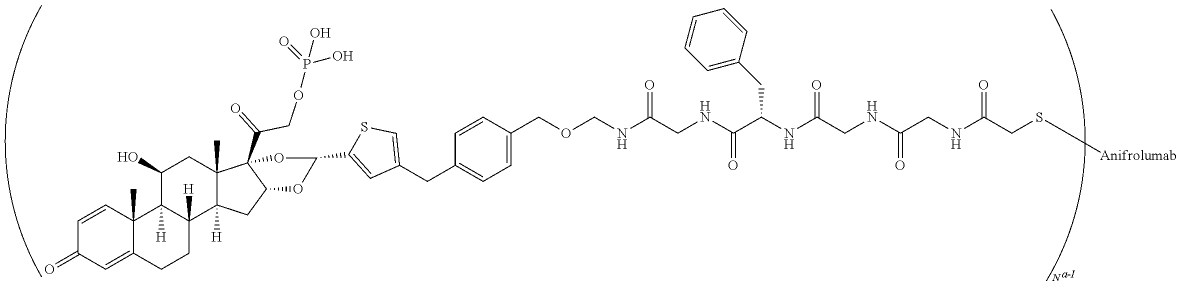 |
| II-C- 1216 | 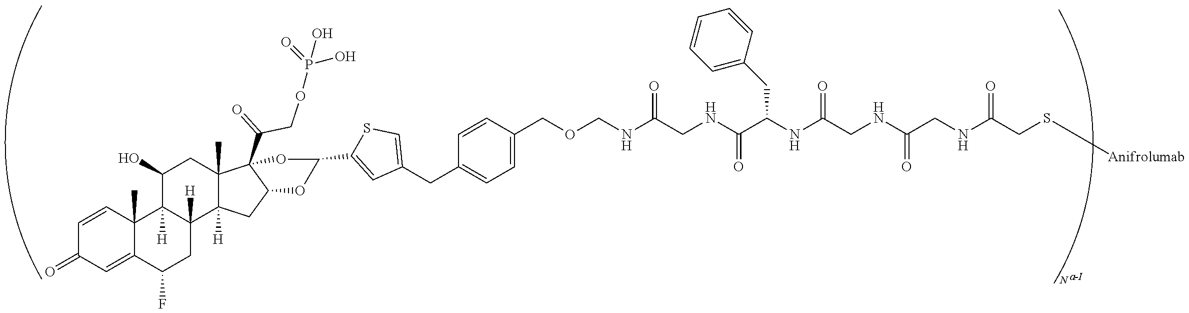 |
| II-C- 1217 | 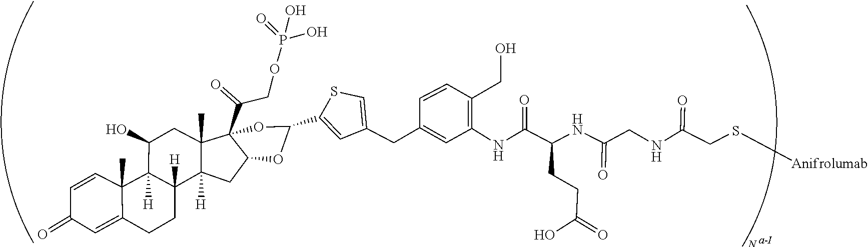 |
| II-C- 1220 | 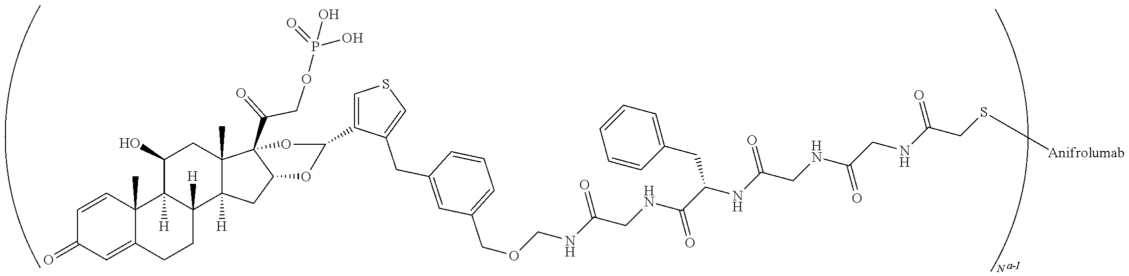 |
| II-C- 1221 | 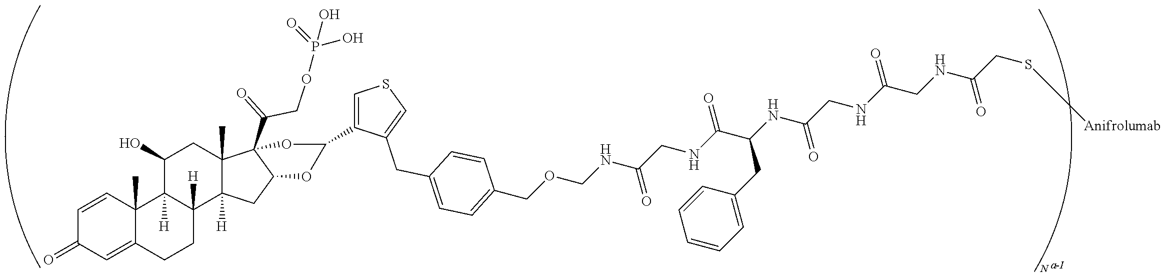 |

In another aspect, the present application provides a conjugate that may comprise the compound or the tautomer, the mesomer, the racemate, the enantiomer or the diastereoisomer thereof, or the mixture thereof, or the pharmaceutically acceptable salt thereof described above.

For example, the conjugate may comprise a ligand-drug conjugate.

For example, the ligand may comprise an antibody or an antigen-binding fragment thereof:

For example, the antibody may be selected from the group consisting of: a human antibody, a humanized antibody, a chimeric antibody, a multispecific antibody, a monoclonal antibody and a polyclonal antibody.

For example, the antigen-binding fragment may be selected from: a Fab, a Fab′, a F(ab′)₂, an Fv, a scFv, a diabody, an Fd, a dAb, a VHH, a maxibody and a complementarity determining region (CDR) fragment.

For example, the ligand specifically binds to an antigen that may be selected from the group consisting of: AXL, BAFFR, BCMA, BCR-list components, BDCA2, BDCA4, BTLA, BTNL2 BTNL3, BTNL8, BTNL9, C10orf54, CCR1, CCR3, CCR4, CCR5, CCR6, CCR7, CCR9, CCR10, CD11c, CD137, CD138, CD14, CD163, CD168, CD177, CD19, CD20, CD209, CD209L, CD22, CD226, CD248, CD25, CD27, CD274, CD276, CD28, CD30, CD300A, CD33, CD37, CD38, CD4, cluster of differentiation 40 (CD40), CD44, CD45, CD46, CD47, CD48, CD5, CD52, CD55, CD56, CD59, CD62E, CD68, CD69, CD70, CD74, CD79a, CD79b, CD8, CD80, CD86, CD90.2, CD96, CLEC12A, CLEC12B, CLEC7A, CLEC9A, CR1, CR3, CRTAM, CSF1R, CTLA4, CXCR1/2, CXCR4, CXCR5, DDR1, DDR2, DEC-205, DLL4, DR6, FAP, FCamR, FCMR, FcR's, Fire, GITR, HHLA2, HLA class II, HVEM, ICOSLG, IFNAR, type I interferon receptor subunit (IFNAR1), IFNLR1, IL10R1, IL10R2, IL12R, IL13RA1, IL13RA2, IL15R, IL17RA, IL17R_{B}, IL17RC, IL17RE, IL20R1, IL20R2, IL21R, IL22R1, IL22RA, IL23R, IL27R, IL29R, IL2Rg, IL31R, IL36R, IL3RA, IL4R, IL6R, IL5R, IL7R, IL9R, integrins, LAG3, LIFR, MAG/Siglec-4 (sialic acid-binding immunoglobulin-like lectin-4), MMR, MSR1, NCR3LG1, NKG2D, NKp30, NKp46, OX40 (CD134), PDCD1, PROKR1, PVR, PVRIG, PVRL2, PVRL3, RELT, SIGIRR, Siglec-1 (sialic acid-binding immunoglobulin-like lectin-1), Siglec-10, Siglec-5, Siglec-6, Siglec-7, Siglec-8, Siglec-9, SIRPA, SLAMF7, TACI, TCR-list components/assoc, PTCRA, TCRb, CD3z, CD3, TEK, TGFBR1, TGFBR2, TGFBR3, TIGIT, TLR2, TLR4, tumor necrosis factor α (TNFα), TROY, TSLPR, TYRO, VLDLR, VSIG4, IL2R-y and VTCN1.

For example, the ligand may be selected from: an anti-TNFα antibody or an antigen-binding fragment thereof, an anti-CD40 antibody or an antigen-binding fragment thereof, and an anti-IFNAR1 antibody or an antigen-binding fragment thereof:

For example, the anti-TNF-α antibody or the antigen-binding fragment thereof may competitively inhibit the binding of adalimumab to TNF-α. In certain embodiments, the anti-TNF-α antibody or the antigen-binding fragment thereof may bind to the same TNF-α epitope as adalimumab. In certain embodiments, the anti-TNF-α antibody or the antigen-binding fragment thereof may be adalimumab or an antigen-binding fragment thereof: In certain embodiments, the anti-TNF-α antibody or the antigen-binding fragment thereof may be adalimumab.

In the present application, the ligand comprises at least one CDR in the light chain variable region VL of an antibody. The CDRs of the present application may be as defined according to Kabat or Chothia; CDR sequences defined in various ways all fall within the protection scope of the present application.

For example, the anti-TNFα antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the following molecules: adalimumab, infliximab, afelimomab or golimumab. For example, the anti-TNFα antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the heavy chain variable region of the following molecules: adalimumab, infliximab, afelimomab or golimumab.

For example, the HCDR1 of adalimumab (Humira) may comprise an amino acid sequence set forth in SEQ ID NO: 4. The HCDR2 of adalimumab (Humira) may comprise an amino acid sequence set forth in SEQ ID NO: 5. The HCDR3 of adalimumab (Humira) may comprise an amino acid sequence set forth in SEQ ID NO: 6.

For example, the anti-TNFα antibody or the antigen-binding fragment thereof may comprise a heavy chain variable region of the antibody, wherein the heavy chain variable region may have at least about 80%, about 85%, about 90%, about 95%, about 96%, about 97%, about 98% or about 99% sequence identity to the heavy chain variable region of the following molecules: adalimumab, infliximab, afelimomab or golimumab. For example, the heavy chain variable region of adalimumab (Humira) may comprise an amino acid sequence set forth in SEQ ID NO: 8.

For example, the anti-TNFα antibody or the antigen-binding fragment thereof may comprise a heavy chain of the antibody, wherein the heavy chain may have at least about 80%, about 85%, about 90%, about 95%, about 96%, about 97%, about 98% or about 99% sequence identity to the heavy chain of the following molecules: adalimumab, infliximab, afelimomab or golimumab. For example, the heavy chain of adalimumab (Humira) may comprise an amino acid sequence set forth in SEQ ID NO: 10.

For example, the anti-TNFα antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the following molecules: adalimumab, infliximab, afelimomab or golimumab. For example, the anti-TNFα antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the light chain variable region of the following molecules: adalimumab, infliximab, afelimomab or golimumab.

For example, the LCDR1 of adalimumab (Humira) may comprise an amino acid sequence set forth in SEQ ID NO: 1. The LCDR2 of adalimumab (Humira) may comprise an amino acid sequence set forth in SEQ ID NO: 2. The LCDR3 of adalimumab (Humira) may comprise an amino acid sequence set forth in SEQ ID NO: 3.

For example, the anti-TNFα antibody or the antigen-binding fragment thereof may comprise a light chain variable region of the antibody, wherein the light chain variable region may have at least about 80%, about 85%, about 90%, about 95%, about 96%, about 97%, about 98% or about 99% sequence identity to the light chain variable region of the following molecules: adalimumab, infliximab, afelimomab or golimumab. For example, the light chain variable region of adalimumab (Humira) may comprise an amino acid sequence set forth in SEQ ID NO: 7.

For example, the anti-TNFα antibody or the antigen-binding fragment thereof may comprise a light chain of the antibody, wherein the light chain may have at least about 80%, about 85%, about 90%, about 95%, about 96%, about 97%, about 98% or about 99% sequence identity to the light chain of the following molecules: adalimumab, infliximab, afelimomab or golimumab.

For example, the light chain of adalimumab (Humira) may comprise an amino acid sequence set forth in SEQ ID NO: 9.

For example, the anti-TNFα antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the anti-mouse TNFα mIgG2a 8C11.

For example, the anti-TNFα antibody or the antigen-binding fragment thereof may comprise a heavy chain variable region of the antibody, wherein the heavy chain variable region may have at least about 80%, about 85%, about 90%, about 95%, about 96%, about 97%, about 98% or about 99% sequence identity to the heavy chain variable region of the anti-mouse TNFα mIgG2a 8C11. For example, the heavy chain of the anti-mouse TNFα mIgG2a 8C11 may comprise an amino acid sequence set forth in SEQ ID NO: 32.

For example, the anti-TNFα antibody or the antigen-binding fragment thereof may comprise a heavy chain of the antibody, wherein the heavy chain may have at least about 80%, about 85%, about 90%, about 95%, about 96%, about 97%, about 98% or about 99% sequence identity to the heavy chain of the anti-mouse TNFα mIgG2a 8C11.

For example, the anti-TNFα antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the anti-mouse TNFα mIgG2a 8C11.

For example, the light chain of the anti-mouse TNFα mIgG2a 8C11 may comprise an amino acid sequence set forth in SEQ ID NO: 31.

For example, the anti-TNFα antibody or the antigen-binding fragment thereof may comprise a light chain variable region of the antibody, wherein the light chain variable region may have at least about 80%, about 85%, about 90%, about 95%, about 96%, about 97%, about 98% or about 99% sequence identity to the light chain variable region of the anti-mouse TNFα mIgG2a 8C11.

For example, the anti-TNFα antibody or the antigen-binding fragment thereof may comprise a light chain of the antibody, wherein the light chain may have at least about 80%, about 85%, about 90%, about 95%, about 96%, about 97%, about 98% or about 99% sequence identity to the light chain of the anti-mouse TNFα mIgG2a 8C11.

In an embodiment, the antibody or the antigen-binding moiety thereof is an antagonist antibody or an antigen-binding moiety thereof that causes a reduction in CD40 activity or function as compared to CD40 activity or function in the absence of the antibody or the antigen-binding moiety thereof. In particular embodiments, the antibody or the antigen-binding moiety thereof is substantially free of agonist activity, i.e., the antibody or the antigen-binding moiety thereof does not cause an increase in the amount of CD40 activity or function as compared to CD40 activity or function in the absence of the antibody or the antigen-binding moiety thereof: In certain embodiments, the anti-CD40 antibody is a polyclonal antibody, a monoclonal antibody, a chimeric antibody, a humanized antibody, a human antibody, or an antigen-binding moiety thereof:

For example, the anti-CD40 antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the following molecule: iscalimab (CFZ533). For example, the HCDR1 of iscalimab may comprise an amino acid sequence set forth in SEQ ID NO: 14. The HCDR2 of iscalimab may comprise an amino acid sequence set forth in SEQ ID NO: 15. The HCDR3 of iscalimab may comprise an amino acid sequence set forth in SEQ ID NO: 16.

For example, the anti-CD40 antibody or the antigen-binding fragment thereof may comprise a heavy chain variable region of the antibody, wherein the heavy chain variable region may have at least about 80%, about 85%, about 90%, about 95%, about 96%, about 97%, about 98% or about 99% sequence identity to the heavy chain variable region of the following molecule: iscalimab (CFZ533). For example, the heavy chain variable region of iscalimab may comprise an amino acid sequence set forth in SEQ ID NO: 18.

For example, the anti-CD40 antibody or the antigen-binding fragment thereof may comprise a heavy chain of the antibody, wherein the heavy chain may have at least about 80%, about 85%, about 90%, about 95%, about 96%, about 97%, about 98% or about 99% sequence identity to the heavy chain of the following molecule: iscalimab (CFZ533). For example, the heavy chain of iscalimab may comprise an amino acid sequence set forth in SEQ ID NO: 20.

For example, the anti-CD40 antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the following molecule: iscalimab (CFZ533). For example, the LCDR1 of iscalimab may comprise an amino acid sequence set forth in SEQ ID NO: 11. The LCDR2 of iscalimab may comprise an amino acid sequence set forth in SEQ ID NO: 12. The LCDR3 of iscalimab may comprise an amino acid sequence set forth in SEQ ID NO: 13.

For example, the anti-CD40 antibody or the antigen-binding fragment thereof may comprise a light chain variable region of the antibody, wherein the light chain variable region may have at least about 80%, about 85%, about 90%, about 95%, about 96%, about 97%, about 98% or about 99% sequence identity to the light chain variable region of the following molecule: iscalimab (CFZ533). For example, the light chain variable region of iscalimab may comprise an amino acid sequence set forth in SEQ ID NO: 17.

For example, the anti-CD40 antibody or the antigen-binding fragment thereof may comprise a light chain of the antibody, wherein the light chain may have at least about 80%, about 85%, about 90%, about 95%, about 96%, about 97%, about 98% or about 99% sequence identity to the light chain of the following molecule: iscalimab (CFZ533). For example, the light chain of iscalimab may comprise an amino acid sequence set forth in SEQ ID NO: 19.

For example, the antibodies of the present application are specific for (i.e., specifically binds to) IFNART (interferon-a/D receptor 1). Such antibodies may also be referred to herein as “anti-IFNART antibodies of the present application”. In another embodiment, the antibodies of the present application may be specific for human IFNART. In another embodiment, the anti-IFNART antibodies of the present application may cross-react with IFNART of a species other than human or of other proteins structurally related to human IFNART (e.g., human IFNART homologs). In other embodiments, the anti-IFNART antibodies of the present application may be specific for human IFNART only and do not exhibit cross-reactivity with species or other types of cross-reactivity.

For example, the anti-IFNART antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the following molecule: anifrolumab (MEDI-546). For example, the HCDR1 of anifrolumab may comprise an amino acid sequence set forth in SEQ ID NO: 24. The HCDR2 of anifrolumab may comprise an amino acid sequence set forth in SEQ ID NO: 25. The HCDR3 of anifrolumab may comprise an amino acid sequence set forth in SEQ ID NO: 26.

For example, the anti-IFNART antibody or the antigen-binding fragment thereof may comprise a heavy chain variable region of the antibody, wherein the heavy chain variable region may have at least about 80%, about 85%, about 90%, about 95%, about 96%, about 97%, about 98% or about 99% sequence identity to the heavy chain variable region of the following molecule: anifrolumab (MEDI-546). For example, the heavy chain variable region of anifrolumab may comprise an amino acid sequence set forth in SEQ ID NO: 28.

For example, the anti-IFNAR1 antibody or the antigen-binding fragment thereof may comprise a heavy chain of the antibody, wherein the heavy chain may have at least about 80%, about 85%, about 90%, about 95%, about 96%, about 97%, about 98% or about 99% sequence identity to the heavy chain of the following molecule: anifrolumab (MEDI-546). For example, the heavy chain of anifrolumab may comprise an amino acid sequence set forth in SEQ ID NO: 30.

For example, the anti-IFNAR1 antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the following molecule: anifrolumab (MEDI-546). For example, the LCDR1 of anifrolumab may comprise an amino acid sequence set forth in SEQ ID NO: 21. The LCDR2 of anifrolumab may comprise an amino acid sequence set forth in SEQ ID NO: 22. The LCDR3 of anifrolumab may comprise an amino acid sequence set forth in SEQ ID NO: 23.

For example, the anti-IFNAR1 antibody or the antigen-binding fragment thereof may comprise a light chain variable region of the antibody, wherein the light chain variable region may have at least about 80%, about 85%, about 90%, about 95%, about 96%, about 97%, about 98% or about 99% sequence identity to the light chain variable region of the following molecule: anifrolumab (MEDI-546). For example, the light chain variable region of anifrolumab may comprise an amino acid sequence set forth in SEQ ID NO: 27.

For example, the anti-IFNAR1 antibody or the antigen-binding fragment thereof may comprise a light chain of the antibody, wherein the light chain may have at least about 80%, about 85%, about 90%, about 95%, about 96%, about 97%, about 98% or about 99% sequence identity to the light chain of the following molecule: anifrolumab (MEDI-546). For example, the light chain of anifrolumab may comprise an amino acid sequence set forth in SEQ ID NO: 29.

For example, the anti-BDCA2 antibody or the antigen-binding fragment thereof may comprise a heavy chain variable region (VH) of the antibody, wherein the heavy chain variable region may comprise an HCDR1, an HCDR2 and an HCDR3 that may have at least about 80%, about 85%, about 90%, about 95%, about 96%, about 97%, about 98% or about 99% sequence identity to an HCDR1, an HCDR2 and an HCDR3, respectively, of the following molecule: litifilimab (BIIB059). For example, the HCDR1 of litifilimab (BIIB059) may comprise an amino acid sequence set forth in SEQ ID NO: 35. The HCDR2 of litifilimab (BIIB059) may comprise an amino acid sequence set forth in SEQ ID NO: 34. The HCDR3 of litifilimab (BIIB059) may comprise an amino acid sequence set forth in SEQ ID NO: 33.

For example, the anti-BDCA2 antibody or the antigen-binding fragment thereof may comprise a heavy chain variable region of the antibody, wherein the heavy chain variable region may have at least about 80%, about 85%, about 90%, about 95%, about 96%, about 97%, about 98% or about 99% sequence identity to the heavy chain variable region of the following molecule: litifilimab (BIIB059). For example, the heavy chain variable region of litifilimab (BIIB059) may comprise an amino acid sequence set forth in SEQ ID NO: 36.

For example, the anti-BDCA2 antibody or the antigen-binding fragment thereof may comprise a heavy chain of the antibody, wherein the heavy chain may have at least about 80%, about 85%, about 90%, about 95%, about 96%, about 97%, about 98% or about 99% sequence identity to the heavy chain of the following molecule: litifilimab (BIIB059). For example, the heavy chain of litifilimab (BIIB059) may comprise an amino acid sequence set forth in SEQ ID NO: 37.

For example, the anti-BDCA2 antibody or the antigen-binding fragment thereof may comprise a light chain variable region (VL) of the antibody, wherein the light chain variable region may comprise an LCDR1, an LCDR2 and an LCDR3 that may have at least about 80%, about 85%, about 90%, about 95%, about 96%, about 97%, about 98% or about 99% sequence identity to an LCDR1, an LCDR2 and an LCDR3, respectively, of the following molecule: litifilimab (BIIB059). For example, the LCDR1 of litifilimab (BIIB059) may comprise an amino acid sequence set forth in SEQ ID NO: 40. The LCDR2 of litifilimab (BIIB059) may comprise an amino acid sequence set forth in SEQ ID NO: 39. The LCDR3 of litifilimab (BIIB059) may comprise an amino acid sequence set forth in SEQ ID NO: 38.

For example, the anti-BDCA2 antibody or the antigen-binding fragment thereof may comprise a light chain variable region of the antibody, wherein the light chain variable region may have at least about 80%, about 85%, about 90%, about 95%, about 96%, about 97%, about 98% or about 99% sequence identity to the light chain variable region of the following molecule: litifilimab (BIIB059). For example, the light chain variable region of litifilimab (B11B059) may comprise an amino acid sequence set forth in SEQ ID NO: 41.

For example, the anti-BDCA2 antibody or the antigen-binding fragment thereof may comprise a light chain of the antibody, wherein the light chain may have at least about 80%, about 85%, about 90%, about 95%, about 96%, about 97%, about 98% or about 99% sequence identity to the light chain of the following molecule: litifilimab (BIIB059). For example, the light chain of litifilimab (B1II1B059) may comprise an amino acid sequence set forth in SEQ ID NO: 42.

For example, the ligand may be selected from the group consisting of: adalimumab, iscalimab (CFZ533), anifrolumab (MEDI-546), infliximab, afelimomab, golimumab, anti-mouse TNFα mIgG2a 8C11, litifilimab (B1II1B059), and derivatives and biosimilars thereof: The present application also encompasses variants and equivalents that are substantially homologous to the anti-TNF-α antibody, the anti-CD40 antibody or the anti-IFNAR1 antibody set forth herein. Such variants and equivalents may contain, for example, conservative substitution mutations, i.e., substitution of one or more amino acids with a similar amino acid. For example, the conservative substitution may refer to substitution of an amino acid with another amino acid of the same class, such as substitution of an acidic amino acid with another acidic amino acid, substitution of a basic amino acid with another basic amino acid, or substitution of a neutral amino acid with another neutral amino acid. The purpose of conservative amino acid substitutions is well known in the art. The antibody may be a recombinant polypeptide, a natural polypeptide or a synthetic polypeptide of the antibody. It will be appreciated in the art that some amino acid sequences of the present application may be altered without significantly affecting the structure or function of the protein. Thus, the present application may further comprise variants of the polypeptides, which exhibit significant activity or which comprise regions of the antibodies. Such mutants include deletions, insertions, inversions, repeats and type substitutions.

Prevention and/or Treatment of Diseases

The compounds described herein may influence the activity of immune cells. The influence on the activity may be that: compared with in a culture medium of immune cells to which a negative control or a control drug is added, the activity of the immune cells is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more in a culture medium to which the compound of the present application is added. For example, the influence on the activity may be an IC₅₀ value (nM) for immune cells of 10000 or less, 5000 or less, 4000 or less, 3000 or less, 2000 or less, 1000 or less, 500 or less, 400 or less, 300 or less, 200 or less, 150 or less, 120 or less, 110 or less, 100 or less, 99 or less, 98 or less, 97 or less, 95 or less, 90 or less, 80 or less, 75 or less, 70 or less, 65 or less, 62 or less, 60 or less, 50 or less, 40 or less, 30 or less, 25 or less, 23 or less, 22 or less, 20 or less, 19 or less, 18 or less, 18.5 or less, 17 or less, 15 or less, 12 or less, 10 or less, 9 or less, 8.5 or less, 7 or less, 6.7 or less, 6 or less, 5.9 or less, 5.5 or less, 5.0 or less, 4.8 or less, 4.5 or less, 4.4 or less, 4 or less, 3.5 or less, 3 or less, 2.5 or less, 2 or less, 1.5 or less, 1.0 or less, 0.5 or less, 0.3 or less, 0.29 or less, 0.25 or less, 0.21 or less, 0.20 or less, 0.18 or less, 0.17 or less, 0.15 or less, 0.12 or less, 0.10 or less, 0.09 or less, 0.08 or less, 0.07 or less, 0.06 or less, 0.05 or less, 0.04 or less, 0.03 or less, 0.02 or less, or 0.01 or less. For example, the immune cells may include, but are not limited to, granulocytes and/or agranulocytes. For example, the immune cells include, but are not limited to, B cells, T cells, natural killer cells, monocytes, macrophages, mast cells, and/or dendritic cells. The compounds described herein may have a targeting effect. The targeting effect may be that: compared with in a culture medium of immune cells to which a negative control or a control drug is added, the activity of the targeted immune cells is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more in a culture medium to which the compound of the present application is added. For example, the influence on the activity may be an IC₅₀ value (nM) for immune cells of 10000 or less, 5000 or less, 4000 or less, 3000 or less, 2000 or less, 1000 or less, 500 or less, 400 or less, 300 or less, 200 or less, 150 or less, 120 or less, 110 or less, 100 or less, 99 or less, 98 or less, 97 or less, 95 or less, 90 or less, 80 or less, 75 or less, 70 or less, 65 or less, 62 or less, 60 or less, 50 or less, 40 or less, 30 or less, 25 or less, 23 or less, 22 or less, 20 or less, 19 or less, 18 or less, 18.5 or less, 17 or less, 15 or less, 12 or less, 10 or less, 9 or less, 8.5 or less, 7 or less, 6.7 or less, 6 or less, 5.9 or less, 5.5 or less, 5.0 or less, 4.8 or less, 4.5 or less, 4.4 or less, 4 or less, 3.5 or less, 3 or less, 2.5 or less, 2 or less, 1.5 or less, 1.0 or less, 0.5 or less, 0.3 or less, 0.29 or less, 0.25 or less, 0.21 or less, 0.20 or less, 0.18 or less, 0.17 or less, 0.15 or less, 0.12 or less, 0.10 or less, 0.09 or less, 0.08 or less, 0.07 or less, 0.06 or less, 0.05 or less, 0.04 or less, 0.03 or less, 0.02 or less, or 0.01 or less. For example, the immune cells may include, but are not limited to, granulocytes and/or agranulocytes. For example, the immune cells include, but are not limited to, B cells, T cells, natural killer cells, monocytes, macrophages, mast cells, and/or dendritic cells.

The compound described herein may have plasma stability. The plasma stability may be that: the compound of the present application releases no more than 50%, no more than 40%, no more than 30%, no more than 20%, no more than 10%, no more than 7%, no more than 5%, no more than 4%, no more than 3%, no more than 2%, no more than 1.9%, no more than 1.8%, no more than 1.7%, no more than 1.6%, no more than 1.5%, no more than 1.4%, no more than 1.3%, no more than 1.2%, no more than 1.1%, no more than 1.0%, no more than 0.9%, no more than 0.8%, no more than 0.7%, no more than 0.6%, no more than 0.5%, no more than 0.4%, no more than 0.3%, no more than 0.2% or no more than 0.1% of the drug 1 day, 3 days, 5 days, 7 days, 14 days, 20 days or 30 days after the compound is added to plasma.

The compound described herein may have good in vivo safety. The in vivo safety may be that: after the compound of the present application is administered to an animal, the release rate of in vivo free drug in the animal is no more than 50%, no more than 40%, no more than 30%, no more than 20%, no more than 10%, no more than 7%, no more than 5%, no more than 4%, no more than 3%, no more than 2%, no more than 1.9%, no more than 1.8%, no more than 1.7%, no more than 1.6%, no more than 1.5%, no more than 1.4%, no more than 1.3%, no more than 1.2%, no more than 1.10%, no more than 1.0%, no more than 0.9%, no more than 0.8%, no more than 0.7%, no more than 0.6%, no more than 0.5%, no more than 0.4%, no more than 0.3%, no more than 0.2%, or no more than 0.1%. For example, the in vivo safety may be that: the compound described herein may be administered at a concentration of 0.5 mg/kg or more, 1 mg/kg or more, 2 mg/kg or more, 3 mg/kg or more, 4 mg/kg or more, 5 mg/kg or more, 10 mg/kg or more, 20 mg/kg or more, 30 mg/kg or more, 50 mg/kg or more, 70 mg/kg or more, 100 mg/kg or more, 200 mg/kg or more, 500 mg/kg or more, or 1000 mg/kg or more without causing toxic manifestation in the animal. For example, the animal may include, but is not limited to, a cat, a dog, a horse, a pig, a cow, a sheep, a rabbit, a mouse, a rat, a monkey or a human. The administration may include, but is not limited to, oral administration, intravenous injection, intravenous drip, intraperitoneal injection or topical administration.

The compounds described herein may influence the ability of immune cells to release cytokines. The influence on the activity may be that: compared with the situation that a negative control or a control drug is added, the ability of immune cells to release cytokines is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more in a culture medium to which the compound of the present application is added or when the compound of the present application is administered to a subject. For example, the immune cells may include, but are not limited to, granulocytes and/or agranulocytes. For example, the immune cells include, but are not limited to, B cells, T cells, natural killer cells, monocytes, macrophages, mast cells, and/or dendritic cells.

The compounds described herein may have the ability to influence transcription of IFN signaling pathway responsive genes. The ability to influence transcription of IFN signaling pathway responsive genes may be that: compared with the situation that a negative control or a control drug is added to a culture medium of immune cells, the transcription of IFN signaling pathway responsive genes is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is added or when the compound of the present application is administered to a subject.

The compounds described herein may have the ability to influence the degree of skin fibrosis. The ability to influence the degree of skin fibrosis may be that: compared with the situation that a negative control or a control drug is added, the decrease in the degree of skin fibrosis is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is administered to a subject.

The compounds described herein may influence the proliferation capacity of immune cells. The influence on the proliferation capacity of immune cells may be that: compared with the situation that a negative control or a control drug is added to a culture medium of immune cells, the proportion of immune cells is increased by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is added or when the compound of the present application is administered to a subject. For example, the immune cells may include, but are not limited to, granulocytes and/or agranulocytes. For example, the immune cells include, but are not limited to, B cells, T cells, natural killer cells, monocytes, macrophages, mast cells, and/or dendritic cells.

The compounds described herein may have the ability to influence the collagen content of the skin. The ability to influence the collagen content of the skin may be that: compared with the situation that a negative control or a control drug is added, the decrease in the collagen content of the skin is reduced by 1b % or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is administered to a subject.

The compounds described herein may have the ability to influence the GRE expression level. The ability to influence the GRE expression level may be that: compared with the situation that a negative control or a control drug is added to a culture medium of immune cells, the GRE expression level is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is added or when the compound of the present application is administered to a subject.

The compounds described herein may have the ability to influence contact hypersensitivity. The ability to influence contact hypersensitivity may be that: compared with the situation that a negative control or a control drug is added, the contact hypersensitivity is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is administered to a subject.

The compounds described herein may have the ability to influence skin swelling. The ability to influence skin swelling may be that: compared with the situation that a negative control or a control drug is added, the skin swelling is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is administered to a subject.

The compounds described herein may have the ability to influence arthritic symptoms. The ability to influence arthritic symptoms may be that: compared with the situation that a negative control or a control drug is added, the swelling in the arthritic symptoms is reduced by 1% or more, 2% or more, 4% or more, 5% or more, 8% or more, 10% or more, 15% or more, 18% or more, 20% or more, 25% or more, 40% or more, 50% or more, 60% or more, 70% or more, 80% or more, 90% or more, or 95% or more when the compound of the present application is administered to a subject.

### Pharmaceutical Composition

The pharmaceutical composition described herein may contain, in addition to the active compound, one or more adjuvants, which may be selected from the group consisting of the following ingredients: fillers (diluents), binders, wetting agents, disintegrants, excipients, and the like. Depending on the method of administration, the composition may contain 0.1 wt. % to 99% wt. % of the active compound.

The pharmaceutical composition containing the active ingredient may be in a form suitable for oral administration, such as tablet, troche, lozenge, aqueous or oil suspension, dispersible powder or granule, emulsion, hard or soft capsule, or syrup. Oral compositions may be prepared according to any method for preparing pharmaceutical compositions known in the art, and the compositions may contain binders, fillers, lubricants, disintegrants, pharmaceutically acceptable wetting agents, and the like, and may also contain one or more ingredients that are selected from the group consisting of sweetening agents, flavouring agents, coloring agents and preservatives.

Aqueous suspensions may contain the active substance in admixture with excipients suitable for the formulation of aqueous suspensions. Aqueous suspensions may also contain one or more preservatives, for example, one or more coloring agents, one or more flavoring agents, and one or more sweetening agents. Oily suspensions may be formulated by suspending the active ingredient in a vegetable oil. These oil suspensions may contain thickening agents. The sweetening agents and the flavoring agents described above may also be added.

The pharmaceutical compositions may also be prepared as follows: dispersible powders or granules for preparing aqueous suspensions provide the active ingredient, and water is added to mix the active ingredient with one or more of dispersing agents, wetting agents, suspending agents or preservatives. Other excipients, such as sweetening agents, flavoring agents and coloring agents, may also be added. These compositions are well preserved by the addition of antioxidants such as ascorbic acid. The pharmaceutical composition of the present application may also be in the form of an oil-in-water emulsion.

The pharmaceutical composition may be in the form of a sterile injectable aqueous solution. Available and acceptable vehicles or solvents include water, Ringer's solution and isotonic sodium chloride solution. The sterile injectable formulation may be a sterile injectable oil-in-water microemulsion in which the active ingredient is dissolved in the oil phase. For example, the active ingredient is dissolved in a mixture of soybean oil and lecithin. The oil solution may then be added to a mixture of water and glycerol and treated to form a microemulsion. The injection or microemulsion can be locally injected into the bloodstream of a patient in large quantities. Alternatively, it may be desirable to administer solutions and microemulsions in such a way as to maintain a constant circulating concentration of the compound of the present application. To maintain such a constant concentration, a continuous intravenous delivery device may be used. For example, the device may be a Deltec CADD-PLUS™ 5400 intravenous injection pump.

The pharmaceutical composition may be in the form of a sterile injectable aqueous or oily suspension for intramuscular and subcutaneous administration. The suspension may be prepared according to the known art using the suitable dispersing agents or wetting agents and suspending agents described above. The sterile injectable formulation may also be a sterile injection or suspension prepared in a parenterally acceptable non-toxic diluent or solvent. Alternatively, a sterile fixed oil may be conveniently used as a solvent or a suspending medium. The compound of the present application may be administered in the form of a suppository for rectal administration. Such a pharmaceutical composition can be prepared by mixing a drug with a suitable non-irritating excipient which is a solid at ambient temperature but a liquid in the rectum and therefore will melt in the rectum to release the drug. Such materials include cocoa butter, glycerinated gelatin, hydrogenated vegetable oils, and mixtures of polyethylene glycols of various molecular weights and fatty acid esters of polyethylene glycol.

As is well known to those skilled in the art, the dose of the drug administered depends on a variety of factors, including but not limited to, the activity of the particular compound employed, the age of the patient, the weight of the patient, the health condition of the patient, the behavior of the patient, the diet of the patient, the time of administration, the mode of administration, the rate of excretion, the combination of drugs, and the like. In addition, the optimal treatment regimen, such as the mode of treatment, the compound or the tautomer, the mesomer, the racemate, the enantiomer or the diastereoisomer thereof, or the mixture thereof, or the pharmaceutically acceptable salt thereof described herein, and/or the daily amount of the compound or the tautomer, the mesomer, the racemate, the enantiomer or the diastereoisomer thereof, or the mixture thereof, or the pharmaceutically acceptable salt thereof, or the type of the pharmaceutically acceptable salt, can be verified according to conventional treatment schemes.

Prophylactic and/or Therapeutic Methods

The present application provides use of the compound or the tautomer, the mesomer, the racemate, the enantiomer or the diastereoisomer thereof, or the mixture thereof, or the pharmaceutically acceptable salt thereof described herein and/or the pharmaceutical composition described herein in preparing a medicament for the prevention and/or treatment of a disease and/or condition. For example, the disease and/or condition described herein comprises a disease and/or condition associated with glucocorticoid receptor signaling. For example, the disease and/or condition described herein is selected from the group consisting of: proliferative diseases and/or conditions, metabolic diseases and/or conditions, inflammatory diseases and/or conditions, and neurodegenerative diseases and/or conditions.

For example, the disease and/or condition described herein may be selected from the group consisting of: systemic autoimmune diseases and/or conditions, blood system-related diseases and/or conditions, neuromuscular system-related diseases and/or conditions, digestive system-related diseases and/or conditions, urological system-related diseases and/or conditions, endocrine gland system-related diseases and/or conditions, cutaneous muscular system-related diseases and/or conditions, and respiratory system-related diseases and/or conditions. For example, the disease and/or condition may be selected from the group consisting of the following systemic autoimmune diseases and/or conditions: rheumatoid arthritis, systemic lupus erythematosus, scleroderma, Sjogren's syndrome, ankylosing spondylitis, Wegener granulomatosis, and systemic sclerosis. For example, the disease and/or condition may be selected from the group consisting of the following blood system-related diseases and/or conditions: autoimmune hemolytic anemia, pernicious anemia, idiopathic thrombocytopenic purpura, idiopathic thrombocytopenia, and vasculitis. For example, the disease and/or condition may be selected from the group consisting of the following neuromuscular system-related diseases and/or conditions: multiple sclerosis, myasthenia gravis, and Guillain-Barre syndrome. For example, the disease and/or condition may be selected from the group consisting of the following digestive system-related diseases and/or conditions: ulcerative colitis, Crohn's disease, autoimmune liver disease, and atrophic gastritis. For example, the disease and/or condition may be selected from the group consisting of the following urological system-related diseases and/or conditions: IgA nephropathy, primary nephrotic syndrome, autoimmune glomerulonephritis, Goodpasture's syndrome, and lupus nephritis. For example, the disease and/or condition may be selected from the group consisting of the following endocrine gland system-related diseases and/or conditions: type I diabetes, Grave's disease, Hashimoto thyroiditis, primary adrenocortical atrophy, and chronic thyroiditis. For example, the disease and/or condition may be selected from the group consisting of the following cutaneous muscular system-related diseases and/or conditions: psoriasis, pemphigus vulgaris, cutaneous lupus erythematosis, dermatomyositis, and polymyalgia rheumatica. For example, the disease and/or condition may be a respiratory system-related disease and/or condition; for example, the respiratory system-related disease and/or condition may be asthma. For example, the disease and/or condition of the present application may be selected from the group consisting of: asthma, rheumatoid arthritis, ankylosing spondylitis, axial spondyloarthritis, systemic lupus erythematosus, lupus nephritis, cutaneous lupus erythematosus, psoriasis, psoriatic arthritis (PsA), juvenile idiopathic arthritis (JIA), dermatomyositis, scleroderma, multiple sclerosis, atopic dermatitis (AD), myasthenia gravis, primary adrenocortical atrophy, chronic thyroiditis, juvenile-onset diabetes, inflammatory bowel disease, ulcerative colitis, Crohn's disease, autoimmune glomerulonephritis, Goodpasture's syndrome, autoimmune hemolytic anemia, idiopathic thrombocytopenic purpura, idiopathic thrombocytopenia, Sjogren's syndrome, systemic sclerosis (SSc), polymyalgia rheumatica (PMR), vasculitis, hidradenitis suppurativa, Hashimoto thyroiditis, primary myxoedema, Grave's disease, autoimmune atrophic gastritis, insulin dependent diabetes mellitus, Wegener's granulomatosis, discoid lupus erythematosus, IgA nephritis, Behcet's disease, and nonalcoholic steatohepatitis (NASH).

The present application provides the compound or the tautomer, the mesomer, the racemate, the enantiomer or the diastereoisomer thereof, or the mixture thereof, or the pharmaceutically acceptable salt thereof described herein and/or the pharmaceutical composition described herein for use in the prevention and/or treatment of a disease and/or condition. For example, the disease and/or condition described herein comprises a disease and/or condition associated with glucocorticoid receptor signaling. For example, the disease and/or condition described herein is selected from the group consisting of: proliferative diseases and/or conditions, metabolic diseases and/or conditions, inflammatory diseases and/or conditions, and neurodegenerative diseases and/or conditions. For example, the disease and/or condition described herein may be selected from the group consisting of: systemic autoimmune diseases and/or conditions, blood system-related diseases and/or conditions, neuromuscular system-related diseases and/or conditions, digestive system-related diseases and/or conditions, urological system-related diseases and/or conditions, endocrine gland system-related diseases and/or conditions, cutaneous muscular system-related diseases and/or conditions, and respiratory system-related diseases and/or conditions. For example, the disease and/or condition may be selected from the group consisting of the following systemic autoimmune diseases and/or conditions: rheumatoid arthritis, systemic lupus erythematosus, scleroderma, Sjogren's syndrome, ankylosing spondylitis, Wegener granulomatosis, and systemic sclerosis.

For example, the disease and/or condition may be selected from the group consisting of the following blood system-related diseases and/or conditions: autoimmune hemolytic anemia, pernicious anemia, idiopathic thrombocytopenic purpura, idiopathic thrombocytopenia, and vasculitis. For example, the disease and/or condition may be selected from the group consisting of the following neuromuscular system-related diseases and/or conditions: multiple sclerosis, myasthenia gravis, and Guillain-Barre syndrome. For example, the disease and/or condition may be selected from the group consisting of the following digestive system-related diseases and/or conditions: ulcerative colitis, Crohn's disease, autoimmune liver disease, and atrophic gastritis. For example, the disease and/or condition may be selected from the group consisting of the following urological system-related diseases and/or conditions: IgA nephropathy, primary nephrotic syndrome, autoimmune glomerulonephritis, Goodpasture's syndrome, and lupus nephritis. For example, the disease and/or condition may be selected from the group consisting of the following endocrine gland system-related diseases and/or conditions: type I diabetes, Grave's disease, Hashimoto thyroiditis, primary adrenocortical atrophy, and chronic thyroiditis. For example, the disease and/or condition may be selected from the group consisting of the following cutaneous muscular system-related diseases and/or conditions: psoriasis, pemphigus vulgaris, cutaneous lupus erythematosis, dermatomyositis, and polymyalgia rheumatica. For example, the disease and/or condition may be a respiratory system-related disease and/or condition; for example, the respiratory system-related disease and/or condition may be asthma. For example, the disease and/or condition of the present application may be selected from the group consisting of: asthma, rheumatoid arthritis, ankylosing spondylitis, axial spondyloarthritis, systemic lupus erythematosus, lupus nephritis, cutaneous lupus erythematosus, psoriasis, psoriatic arthritis (PsA), juvenile idiopathic arthritis (JIA), dermatomyositis, scleroderma, multiple sclerosis, atopic dermatitis (AD), myasthenia gravis, primary adrenocortical atrophy, chronic thyroiditis, juvenile-onset diabetes, inflammatory bowel disease, ulcerative colitis, Crohn's disease, autoimmune glomerulonephritis, Goodpasture's syndrome, autoimmune hemolytic anemia, idiopathic thrombocytopenic purpura, idiopathic thrombocytopenia, Sjogren's syndrome, systemic sclerosis (SSc), polymyalgia rheumatica (PMR), vasculitis, hidradenitis suppurativa, Hashimoto thyroiditis, primary myxoedema, Grave's disease, autoimmune atrophic gastritis, insulin dependent diabetes mellitus, Wegener's granulomatosis, discoid lupus erythematosus, IgA nephritis, Behcet's disease, and nonalcoholic steatohepatitis (NASH).

The present application provides a method for preventing and/or treating a disease and/or condition, which may comprise administering to a subject the compound or the tautomer, the mesomer, the racemate, the enantiomer or the diastereoisomer thereof, or the mixture thereof, or the pharmaceutically acceptable salt thereof described herein and/or the pharmaceutical composition described herein. For example, the disease and/or condition described herein comprises a disease and/or condition associated with glucocorticoid receptor signaling. For example, the disease and/or condition described herein is selected from the group consisting of: proliferative diseases and/or conditions, metabolic diseases and/or conditions, inflammatory diseases and/or conditions, and neurodegenerative diseases and/or conditions.

For example, the disease and/or condition described herein may be selected from the group consisting of: systemic autoimmune diseases and/or conditions, blood system-related diseases and/or conditions, neuromuscular system-related diseases and/or conditions, digestive system-related diseases and/or conditions, urological system-related diseases and/or conditions, endocrine gland system-related diseases and/or conditions, cutaneous muscular system-related diseases and/or conditions, and respiratory system-related diseases and/or conditions. For example, the disease and/or condition may be selected from the group consisting of the following systemic autoimmune diseases and/or conditions: rheumatoid arthritis, systemic lupus erythematosus, scleroderma, Sjogren's syndrome, ankylosing spondylitis, Wegener granulomatosis, and systemic sclerosis. For example, the disease and/or condition may be selected from the group consisting of the following blood system-related diseases and/or conditions: autoimmune hemolytic anemia, pernicious anemia, idiopathic thrombocytopenic purpura, idiopathic thrombocytopenia, and vasculitis. For example, the disease and/or condition may be selected from the group consisting of the following neuromuscular system-related diseases and/or conditions: multiple sclerosis, myasthenia gravis, and Guillain-Barre syndrome. For example, the disease and/or condition may be selected from the group consisting of the following digestive system-related diseases and/or conditions: ulcerative colitis, Crohn's disease, autoimmune liver disease, and atrophic gastritis. For example, the disease and/or condition may be selected from the group consisting of the following urological system-related diseases and/or conditions: IgA nephropathy, primary nephrotic syndrome, autoimmune glomerulonephritis, Goodpasture's syndrome, and lupus nephritis. For example, the disease and/or condition may be selected from the group consisting of the following endocrine gland system-related diseases and/or conditions: type I diabetes, Grave's disease, Hashimoto thyroiditis, primary adrenocortical atrophy, and chronic thyroiditis. For example, the disease and/or condition may be selected from the group consisting of the following cutaneous muscular system-related diseases and/or conditions: psoriasis, pemphigus vulgaris, cutaneous lupus erythematosis, dermatomyositis, and polymyalgia rheumatica. For example, the disease and/or condition may be a respiratory system-related disease and/or condition; for example, the respiratory system-related disease and/or condition may be asthma. For example, the disease and/or condition of the present application may be selected from the group consisting of: asthma, rheumatoid arthritis, ankylosing spondylitis, axial spondyloarthritis, systemic lupus erythematosus, lupus nephritis, cutaneous lupus erythematosus, psoriasis, psoriatic arthritis (PsA), juvenile idiopathic arthritis (JIA), dermatomyositis, scleroderma, multiple sclerosis, atopic dermatitis (AD), myasthenia gravis, primary adrenocortical atrophy, chronic thyroiditis, juvenile-onset diabetes, inflammatory bowel disease, ulcerative colitis, Crohn's disease, autoimmune glomerulonephritis, Goodpasture's syndrome, autoimmune hemolytic anemia, idiopathic thrombocytopenic purpura, idiopathic thrombocytopenia, Sjogren's syndrome, systemic sclerosis (SSc), polymyalgia rheumatica (PMR), vasculitis, hidradenitis suppurativa, Hashimoto thyroiditis, primary myxoedema, Grave's disease, autoimmune atrophic gastritis, insulin dependent diabetes mellitus, Wegener's granulomatosis, discoid lupus erythematosus, IgA nephritis, Behcet's disease, and nonalcoholic steatohepatitis (NASH).

### Technical Schemes for Synthesis

For the synthesis purpose of the present application, the following technical schemes for synthesis are adopted in the present application:

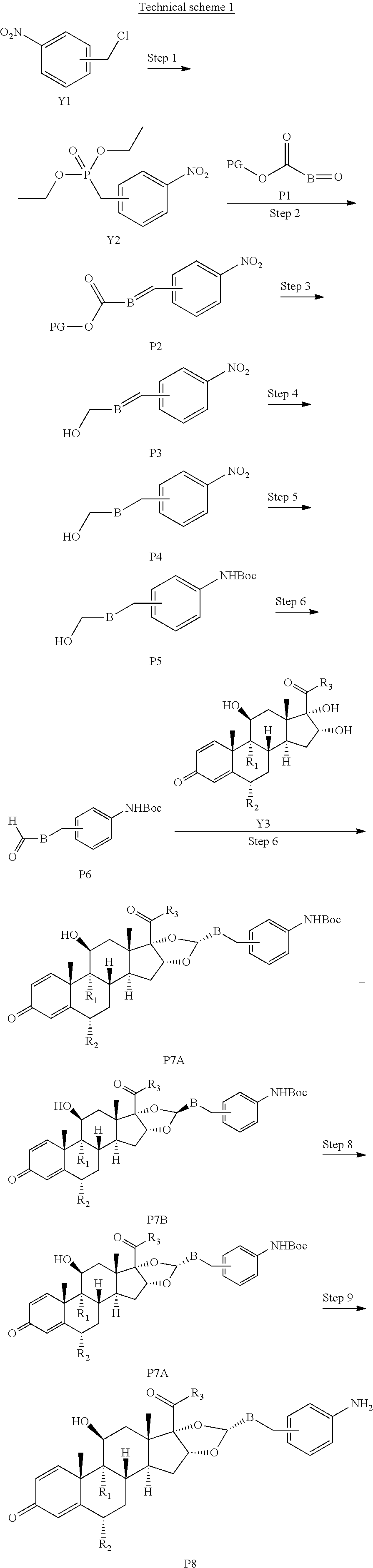

Step 1: reacting a compound of general formula (Y1) under optional basic conditions to obtain a compound of general formula (Y2);
Step 2: reacting a compound of general formula (P1) with the compound of general formula (Y2) under optional basic conditions to obtain a compound of general formula (P2);
Step 3: reacting the compound of general formula (P2) under reducing conditions to obtain a compound of general formula (P3);
Step 4: reacting the compound of general formula (P3) under optional hydrogenation conditions to obtain a compound of general formula (P4);
Step 5: applying the protecting group tert-butyl carbamate to the compound of general formula (P4) under optional basic conditions to obtain a compound of general formula (P5);
Step 6: reacting the compound of general formula (P5) under optional oxidation conditions to obtain a compound of general formula (P6);
Step 7: reacting the compound of general formula (P6) with the compound of general formula (Y3) under optional acidic or basic conditions to obtain a compound of general formula (P7A) and a compound of general formula (P7B);
Step 8: subjecting the diastereoisomer to separation by preparative HPLC to obtain R-configuration isomer (P7A); and
Step 9: removing the protecting group of the compound of general formula (P7A) under optional acidic conditions to obtain a compound of general formula (P8).

In the above steps,
PG may be a common hydroxy, amino, sulfhydryl or carboxy protecting group;
R₁, R₂ and R₃ may be defined as R₁, R₂ and R₃ in any one of formula (I), formula (I-a1), formula (I-a2), formula (II), formula (II-a1), formula (II-a2), formula (II-b1) or formula (II-b2) of the present application;
B may be defined as B of any one of formula (I), formula (I-a1), formula (I-a2), formula (II-a1), formula (II-a2), formula (II-b1) or formula (II-b2) of the present application, or B may be an aliphatic ring or aliphatic heterocyclic ring;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium borohydride, sodium cyanoborohydride, lithium aluminum hydride and sodium hydride;
hydrogenation conditions include, but are not limited to: Pb/C/hydrogen, Pt/C/hydrogen, palladium chloride/hydrogen, Raney nickel/hydrogen, palladium hydroxide carbon/hydrogen, and palladium hydroxide/hydrogen;
reagents that provide oxidation conditions include, but are not limited to, Dess-Martin periodinane, hydrogen peroxide, sodium chlorite, sodium hypochlorite, and potassium perchlorate.

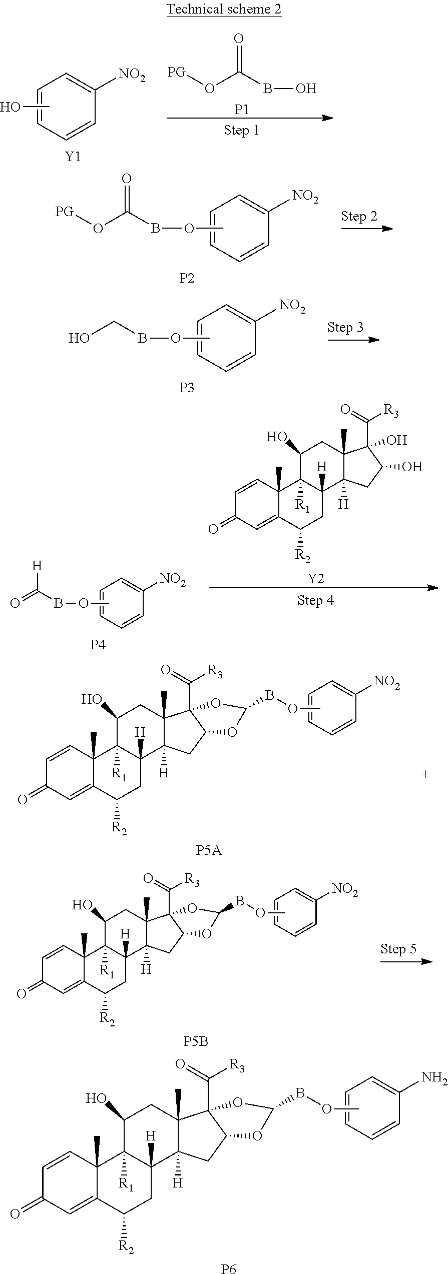

### Ring B is an Aliphatic Ring or Aliphatic Heterocyclic Ring

Step 1: subjecting a compound of general formula (YT) and a compound of general formula (P1) to the Mitsunobu reaction to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain compounds of general formulas (P5A and P5B); and
Step 5: reacting the compounds of general formulas (P5A and P5B) under optional reducing conditions, followed by separation by preparative HPLC, to obtain a compound of general formula (P6).

In the above steps,
R₁, R₂ and R₃ may be defined as R₁, R₂ and R₃ in any one of formula (I), formula (I-a1), formula (I-a2), formula (II), formula (II-a1), formula (II-a2), formula (II-b1) or formula (II-b2) of the present application;
B may be defined as B of any one of formula (I), formula (I-a1), formula (I-a2), formula (II-a1), formula (II-a2), formula (II-b1) or formula (II-b2) of the present application, or B may be an aliphatic ring or aliphatic heterocyclic ring;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium borohydride, sodium cyanoborohydride, lithium aluminum hydride and sodium hydride;
hydrogenation conditions include, but are not limited to: Pb/C/hydrogen, Pt/C/hydrogen, palladium chloride/hydrogen, Raney nickel/hydrogen, palladium hydroxide carbon/hydrogen, and palladium hydroxide/hydrogen;
reagents that provide oxidation conditions include, but are not limited to, Dess-Martin periodinane, hydrogen peroxide, sodium chlorite, sodium hypochlorite, and potassium perchlorate.

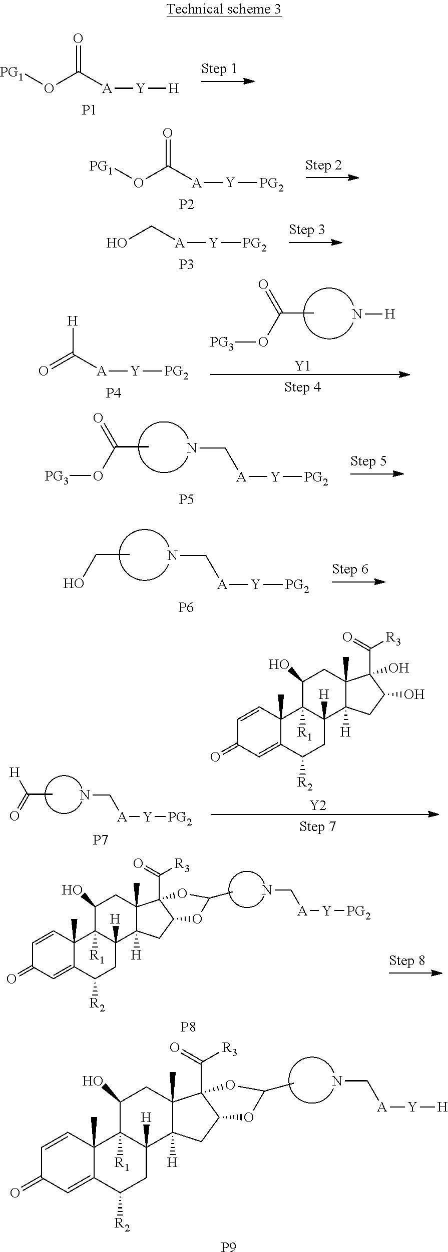

Step 1: subjecting the active group Y(—H) on a compound of general formula (P1) to substitution with a protecting group under optional basic conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: subjecting the compound of general formula (P4) and a compound of general formula (Y1) to the reductive amination reaction to obtain a compound of general formula (P5);
Step 5: reacting the compound of general formula (P5) under optional reducing conditions to obtain a compound of general formula (P6);
Step 6: reacting the compound of general formula (P6) under optional oxidation conditions to obtain a compound of general formula (P7);
Step 7: reacting the compound of general formula (P7) with a compound of general formula (Y2) under optional acidic or basic conditions to obtain a compound of general formula (P8); and
Step 8: removing the protecting group PG2 from the compound of general formula (P8) under optional acidic or basic conditions, followed by separation by preparative HPLC, to obtain a compound of general formula (P9).

In the above steps,
PG₁ and PG₂ may be common hydroxy, amino, sulfhydryl or carboxy protecting groups;
R₁, R₂ and R₃ may be defined as R₁, R₂ and R₃ in any formula (II) of the present application;
A and Y may be defined as A and Y in any formula (II) of the present application, or A may be optionally substituted alkyl, optionally substituted heteroalkyl, optionally substituted aliphatic ring or optionally substituted aliphatic heterocyclic ring;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium borohydride, sodium cyanoborohydride, lithium aluminum hydride and sodium hydride;
hydrogenation conditions include, but are not limited to: Pb/C/hydrogen, Pt/C/hydrogen, palladium chloride/hydrogen, Raney nickel/hydrogen, palladium hydroxide carbon/hydrogen, and palladium hydroxide/hydrogen;
reagents that provide oxidation conditions include, but are not limited to, Dess-Martin periodinane, hydrogen peroxide, sodium chlorite, sodium hypochlorite, and potassium perchlorate.

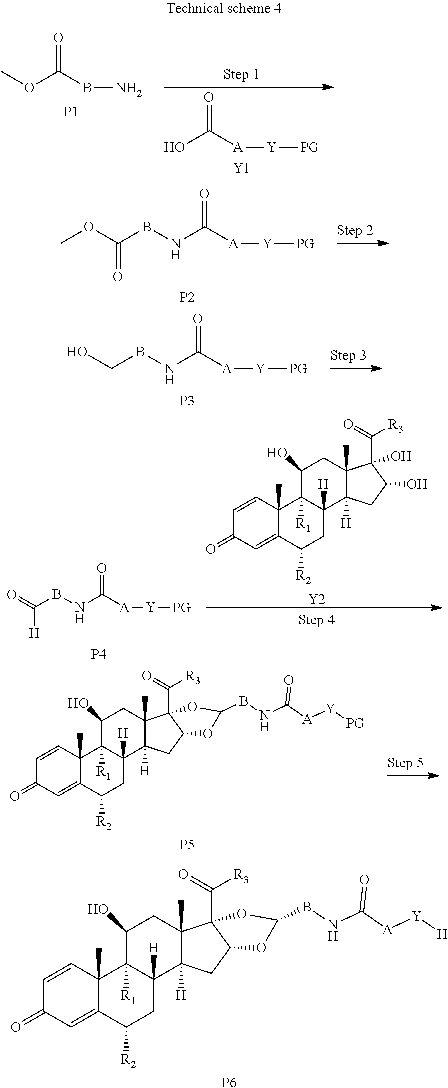

Step 1: reacting a compound of general formula (P1) with a compound of general formula (Y1) under optional condensation conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5); and
Step 5: reacting the compound of general formula (P5) under optional reducing conditions to obtain a compound of general formula (P6).

In the above steps,
PG may be a common hydroxy, amino, sulfhydryl or carboxy protecting group;
R₁, R₂ and R₃ may be defined as R₁, R₂ and R₃ in any formula (II) of the present application;
B, A and Y may be defined as B, A and Y in any formula (II) of the present application, or A may be optionally substituted alkyl, optionally substituted heteroalkyl, optionally substituted aliphatic ring or optionally substituted aliphatic heterocyclic ring;
the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, 0-benzotriazol-N,N,N′,N′-tetramethyluronium tetrafluoroborate, 1-hydroxybenzotriazole, 1-hydroxy-7-azobenzotriazol, 0-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium hydride, calcium hydride, lithium hydride, lithium aluminum hydride, sodium borohydride, lithium borohydride, sodium triethylborohydride, sodium triacetoxyborohydride and sodium cyanoborohydride;
hydrogenation conditions include, but are not limited to: Pb/C/hydrogen, Pt/C/hydrogen, palladium chloride/hydrogen, Raney nickel/hydrogen, palladium hydroxide carbon/hydrogen, and palladium hydroxide/hydrogen;
reagents that provide oxidation conditions include, but are not limited to, Dess-Martin periodinane, hydrogen peroxide, sodium chlorite, sodium hypochlorite, and potassium perchlorate.

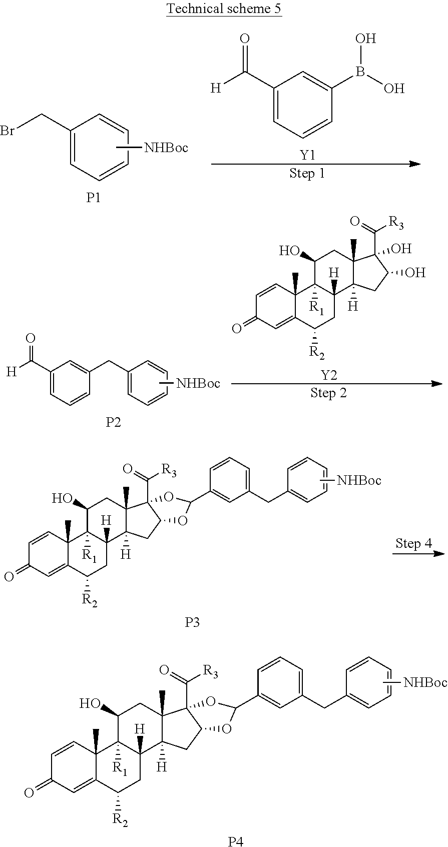

Step 1: reacting a compound of general formula (P1) with a compound of general formula (Y1) under optional palladium reagent catalysis conditions to obtain a compound of general formula (P2);
Step 2: reacting the compound of general formula (P2) with a compound of general formula (Y2) under optional acidic or basic conditions to obtain a compound of general formula (P3); and
Step 3: removing the protecting group tert-butyloxycarbonyl from the compound of general formula (P3) under optional acidic conditions, followed by separation by preparative HPLC, to obtain a compound of general formula (P4).

In the above steps,
R₁, R₂ and R₃ may be defined as R₁, R₂ and R₃ in any formula (I) of the present application; palladium catalysts include, but are not limited to, tetrakis(triphenylphosphine)palladium, palladium acetate, palladium chloride, bis(triphenylphosphine)palladium(II) dichloride, tris(dibenzylideneacetone)dipalladium, bis(dibenzylideneacetone)dipalladium, bis(acetonitrile)palladium(II) dichloride, [1,1′-bis(diphenylphosphino)ferrocene]palladium(II) dichloride, [1,1′-bis(diphenylphosphino)ferrocene]palladium(II) dichloride dichloromethane complex, bis(benzonitrile)palladium chloride, 1,4-butylenebis(diphenylphosphine)-palladium dichloride, allylpalladium chloride dimer, and allyl(cyclopentadienyl)palladium(II);
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

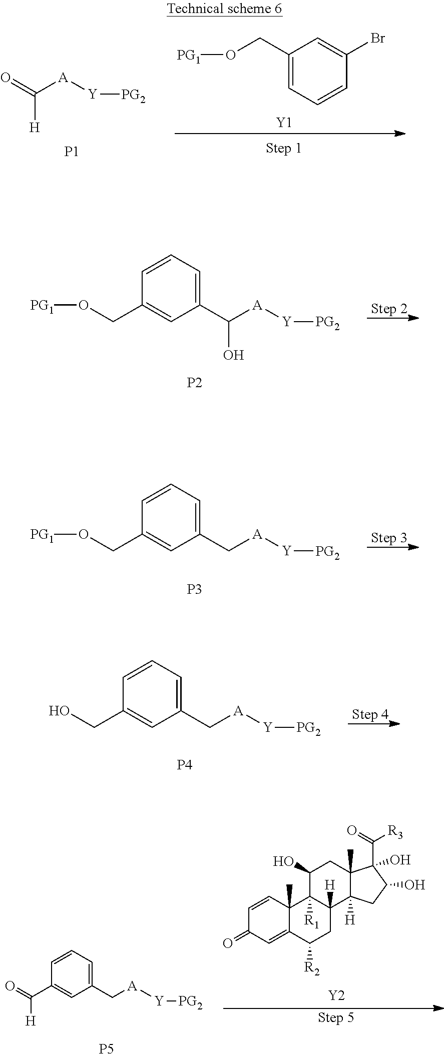

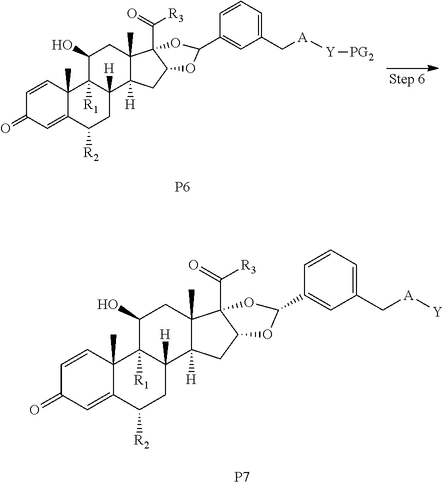

Step 1: subjecting a compound of general formula (P1) and a compound of general formula (Y1) to the Grignard reaction under catalysis of the magnesium metal to obtain a compound of general formula (P2);
Step 2: subjecting the compound of general formula (P2) to the Barton deoxygenation reaction to obtain a compound of general formula (P3);
Step 3: removing the protecting group PG₁ from the compound of general formula (P3) under optional acidic or basic conditions to obtain a compound of general formula (P4);
Step 4: reacting the compound of general formula (P4) under optional oxidation conditions to obtain a compound of general formula (P5);
Step 5: reacting the compound of general formula (P5) with a compound of general formula (Y2) under optional acidic or basic conditions to obtain a compound of general formula (P6); and
Step 6: removing the protecting group PG₂ from the compound of general formula (P6) under optional acidic or basic conditions, followed by separation by preparative HPLC, to obtain a compound of general formula (P7).

In the above steps,
PG₁ and PG₂ may be common hydroxy, amino, sulfhydryl or carboxy protecting groups;
R₁, R₂ and R₃ may be defined as R₁, R₂ and R₃ in any formula (I) of the present application;
A and Y may be defined as A and Y in any formula (I) of the present application, or A may be optionally substituted alkyl, optionally substituted heteroalkyl, optionally substituted aliphatic ring or optionally substituted aliphatic heterocyclic ring;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide oxidation conditions include, but are not limited to, Dess-Martin periodinane, hydrogen peroxide, sodium chlorite, sodium hypochlorite, and potassium perchlorate.

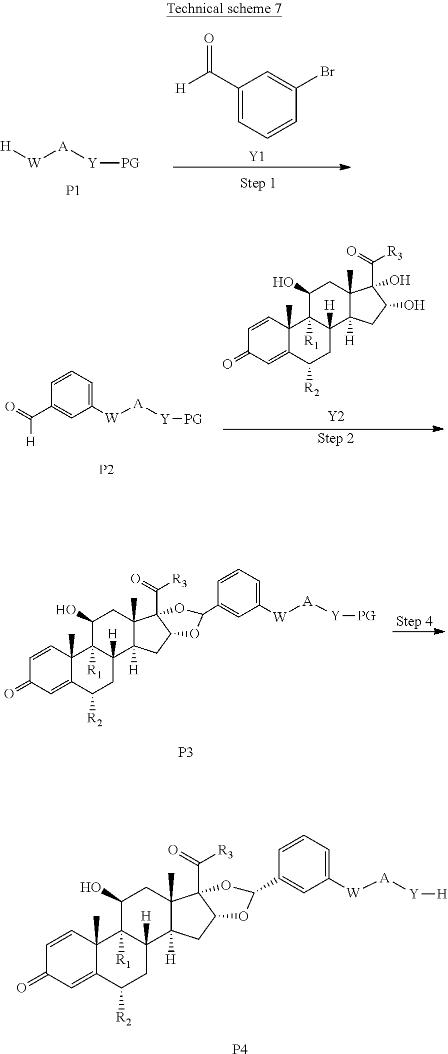

Step 1: reacting a compound of general formula (P1) with a compound of general formula (Y1) under catalysis of optional metallic copper salt and optional ligand and under optional basic conditions to obtain a compound of general formula (P2);
Step 2: reacting the compound of general formula (P2) with a compound of general formula (Y2) under optional acidic or basic conditions to obtain a compound of general formula (P3); and
Step 3: removing the protecting group PG from the compound of general formula (P3) under optional acidic or basic conditions to obtain a compound of general formula (P4).

In the above steps,
PG₁ and PG₂ may be common hydroxy, amino, sulfhydryl or carboxy protecting groups;
R₁, R₂ and R₃ may be defined as R₁, R₂ and R₃ in any formula (I) of the present application;
W, A and Y may be defined as W, A and Y in any formula (I) of the present application;
or A may be optionally substituted alkyl, optionally substituted aliphatic ring, optionally substituted aliphatic heterocyclic ring, optionally substituted aromatic ring or optionally substituted aromatic heterocyclic ring;
or W may be —NR₁—, —O—, —S—, —O—(CR₁R₂)ₙ₁— or —S—(CR₁R₂)ₙ₂—, wherein R, R₁ and R₂ may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or C₁₋₆ aliphatic group, and n1 and n2 may each independently be selected from numbers equal to or greater than 0;
or Y may be —O—, —NR— or —S—, wherein R may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
metallic copper salts include, but are not limited to, copper sulfate, copper sulfate pentahydrate, cuprous sulfate, cupric chloride, cuprous chloride, cupric carbonate, cupric phosphate, cupric acetate, and hydrates thereof, cupric oxalate, cupric fluoroborate, and hydrates thereof, cupric methoxide, cupric tartrate, copper formate, cuprous iodide, copper(II) trifluoroacetate, copper(II) trifluoromethanesulfonate, copper carbonate, cupric bromide, cuprous bromide, and cuprous oxide;
the ligand may be selected from any ligand commonly used in the Ullmann reaction, including but not limited to, L-proline, tyrosine, phenylalanine, 1,10-phenanthroline, N,N′-dimethylethylenediamine, ethylene glycol, 1,1′-binaphthyl-2,2′-diol, ethyl 2-cyclohexanonecarboxylate, salicylaldehyde hydrazone, and the like;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, sodium phosphate, potassium phosphate, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

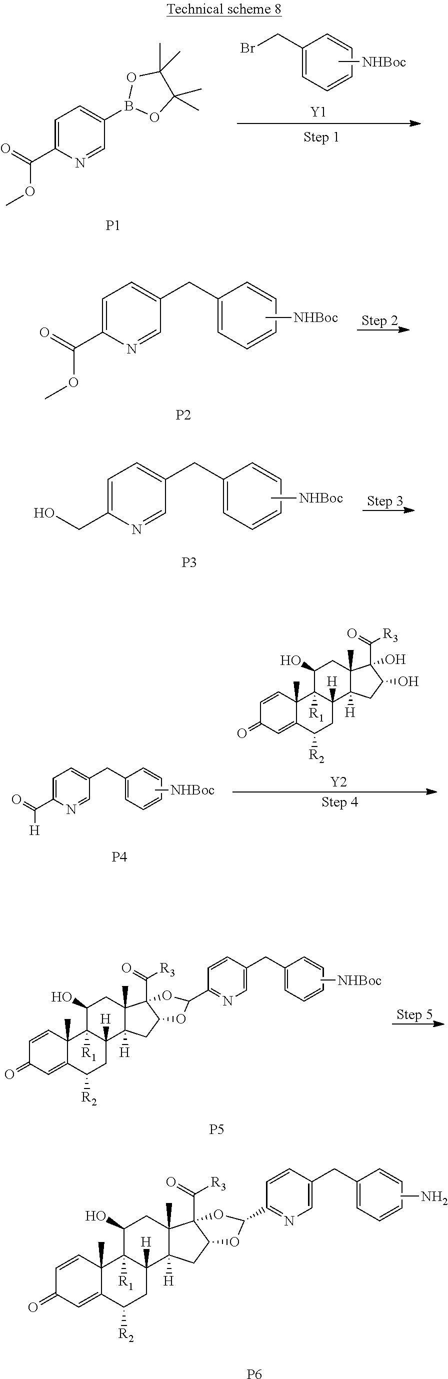

Step 1: reacting a compound of general formula (P1) with a compound of general formula (Y1) under optional palladium reagent catalysis conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5); and
Step 5: removing the protecting group tert-butyloxycarbonyl from the compound of general formula (P5) under optional acidic conditions, followed by separation by preparative HPLC, to obtain a compound of general formula (P6).

In the above steps, R₁, R₂ and R₃ may be defined as R₁, R₂ and R₃ in any one of formula (II), formula (II-a1), formula (II-a2), formula (II-b1) or formula (II-b2) of the present application; palladium catalysts include, but are not limited to, tetrakis(triphenylphosphine)palladium, palladium acetate, palladium chloride, bis(triphenylphosphine)palladium(II) dichloride, tris(dibenzylideneacetone)dipalladium, bis(dibenzylideneacetone)dipalladium, bis(acetonitrile)palladium(II) dichloride, [1,1′-bis(diphenylphosphino)ferrocene]palladium(II) dichloride, [1,1′-bis(diphenylphosphino)ferrocene]palladium(II) dichloride dichloromethane complex, bis(benzonitrile)palladium chloride, 1,4-butylenebis(diphenylphosphine)-palladium dichloride, allylpalladium chloride dimer, and allyl(cyclopentadienyl)palladium(II);
reagents that provide reducing conditions include, but are not limited to, sodium hydride, calcium hydride, lithium hydride, lithium aluminum hydride, sodium borohydride, lithium borohydride, sodium triethylborohydride, sodium triacetoxyborohydride and sodium cyanoborohydride;
reagents that provide oxidation conditions include, but are not limited to, Dess-Martin periodinane, hydrogen peroxide, sodium chlorite, sodium hypochlorite, and potassium perchlorate;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like.

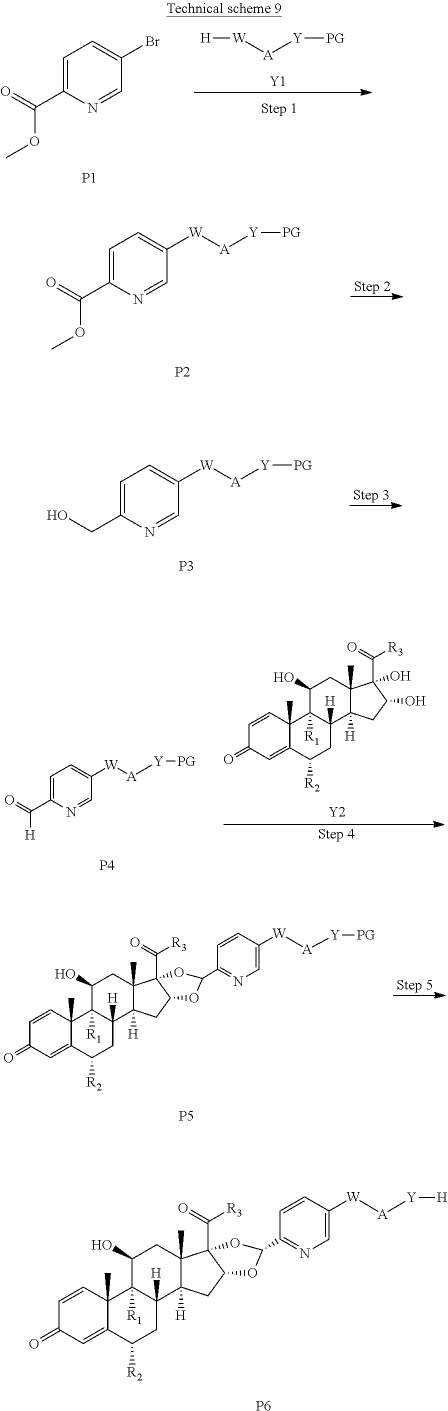

Step 1: reacting a compound of general formula (P1) with a compound of general formula (Y1) under catalysis of optional metallic copper salt and optional ligand and under optional basic conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5); and
Step 5: removing the protecting group PG from the compound of general formula (P5) under optional acidic or basic conditions, followed by separation by preparative HPLC, to obtain a compound of general formula (P6).
   In the above steps,
PG may be a common hydroxy, amino, sulthydryl or carboxy protecting group;
R₁, R₂ and R₃ may be defined as R₁, R₂ and R₃ in any formula (II) of the present application;
W, A and Y may be defined as W, A and Y in any formula (II) of the present application; or A may be optionally substituted alkyl, optionally substituted heteroalkyl, optionally substituted aliphatic ring, optionally substituted aliphatic heterocyclic ring, optionally substituted aromatic ring, optionally substituted aromatic heterocyclic ring, or optionally substituted linear or branched chain structure;
or W may be —NR₁—, —O—, —S—, —O—(CR₁R₂)ₙ₁— or —S—(CR₁R₂)ₙ₂—, wherein R, R₁ and R₂ may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or C₁₋₆ aliphatic group, and n1 and n2 may each independently be selected from numbers equal to or greater than 0;
or Y may be —O—, —NR— or —S—, wherein R may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C₁₋₆ aliphatic group;
metallic copper salts include, but are not limited to, copper sulfate, copper sulfate pentahydrate, cuprous sulfate, cupric chloride, cuprous chloride, cupric carbonate, cupric phosphate, cupric acetate, and hydrates thereof, cupric oxalate, cupric fluoroborate, and hydrates thereof, cupric methoxide, cupric tartrate, copper formate, cuprous iodide, copper(II) trifluoroacetate, copper(II) trifluoromethanesulfonate, copper carbonate, cupric bromide, cuprous bromide, and cuprous oxide;
the ligand may be selected from any ligand commonly used in the Ullmann reaction, including but not limited to, L-proline, tyrosine, phenylalanine, 1,10-phenanthroline, N,N′-dimethylethylenediamine, ethylene glycol, 1,1′-binaphthyl-2,2′-diol, ethyl 2-cyclohexanonecarboxylate, salicylaldehyde hydrazone, and the like;
reagents that provide reducing conditions include, but are not limited to, sodium hydride, calcium hydride, lithium hydride, lithium aluminum hydride, sodium borohydride, lithium borohydride, sodium triethylborohydride, sodium triacetoxyborohydride and sodium cyanoborohydride;
reagents that provide oxidation conditions include, but are not limited to, Dess-Martin periodinane, hydrogen peroxide, sodium chlorite, sodium hypochlorite, and potassium perchlorate;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like.

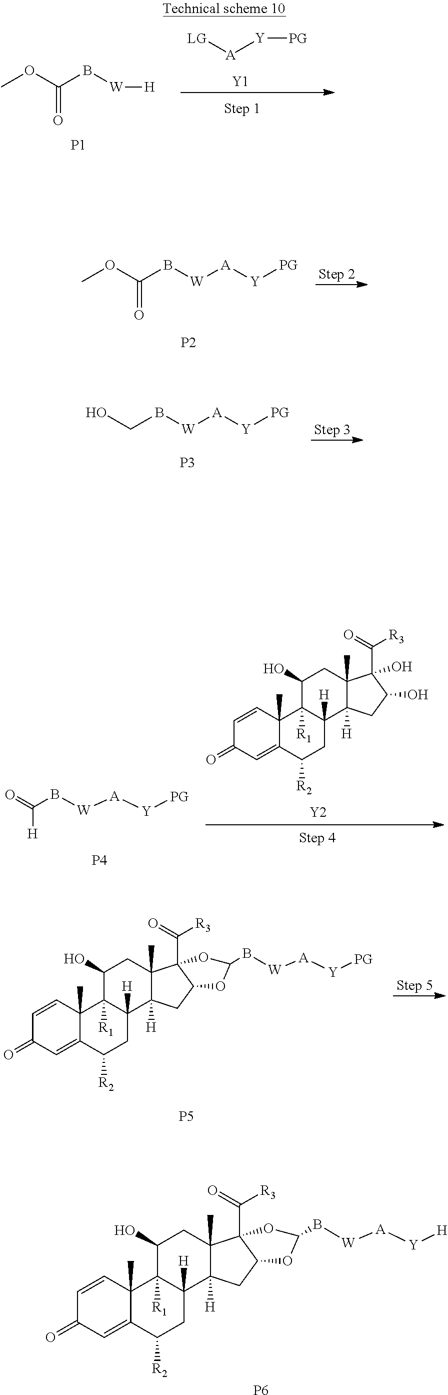

Step 1: reacting a compound of general formula (P1) with a compound of general formula (Y1) under catalysis of optional metallic copper salt, basic conditions and optional ligand or under optional basic conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5); and
Step 5: removing the protecting group PG from the compound of general formula (P5) under optional acidic or basic conditions, followed by separation by preparative HPLC, to obtain a compound of general formula (P6).

In the above steps,
PG may be a common hydroxy, amino, sulfhydryl or carboxy protecting group;
R₁, R₂ and R₃ may be defined as R₁, R₂ and R₃ in any formula (II) of the present application;
B, W, A and Y may be defined as B, W, A and Y in any formula (II) of the present application;
or B may be optionally substituted meta-phenyl ring, optionally substituted pyridine ring, optionally substituted aliphatic ring, or optionally substituted aliphatic heterocyclic ring;
or A may be optionally substituted alkyl, optionally substituted heteroalkyl, optionally substituted aliphatic ring, optionally substituted aliphatic heterocyclic ring, optionally substituted aromatic ring, optionally substituted aromatic heterocyclic ring, or optionally substituted linear or branched chain structure;
or W may be —NR—, —O—, —S—, —O—(CR₁R₂)ₙ₁—, —NR(CR₁R₂)ₙ₂—, —S—(CR₁R₂)ₙ₃—, —(CR₁R₂)ₙ₁—O—, —(CR₁R₂)ₙ₂NR— or —(CR₁R₂)ₙ₃—S—, wherein R, R₁ and R₂ may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or an C₁₋₆ aliphatic group, and n1, n2 and n3 may each independently be selected from numbers equal to or greater than 0;
or Y may be —O—, —NR— or —S—, wherein R may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
LG may be a common leaving group, including but not limited to, fluorine, chlorine, bromine, iodine, trifluoromethanesulfonyloxy, methanesulfonyloxy and p-toluenesulfonyloxy;
metallic copper salts include, but are not limited to, copper sulfate, copper sulfate pentahydrate, cuprous sulfate, cupric chloride, cuprous chloride, cupric carbonate, cupric phosphate, cupric acetate, and hydrates thereof, cupric oxalate, cupric fluoroborate, and hydrates thereof, cupric methoxide, cupric tartrate, copper formate, cuprous iodide, copper(II) trifluoroacetate, copper(II) trifluoromethanesulfonate, copper carbonate, cupric bromide, cuprous bromide, and cuprous oxide;
the ligand may be selected from any ligand commonly used in the Ullmann reaction, including but not limited to, L-proline, tyrosine, phenylalanine, 1,10-phenanthroline, N,N′-dimethylethylenediamine, ethylene glycol, 1,1′-binaphthyl-2,2′-diol, ethyl 2-cyclohexanonecarboxylate, salicylaldehyde hydrazone, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide reducing conditions include, but are not limited to, sodium hydride, calcium hydride, lithium hydride, lithium aluminum hydride, sodium borohydride, lithium borohydride, sodium triethylborohydride, sodium triacetoxyborohydride and sodium cyanoborohydride;
reagents that provide oxidation conditions include, but are not limited to, Dess-Martin periodinane, hydrogen peroxide, sodium chlorite, sodium hypochlorite, and potassium perchlorate.

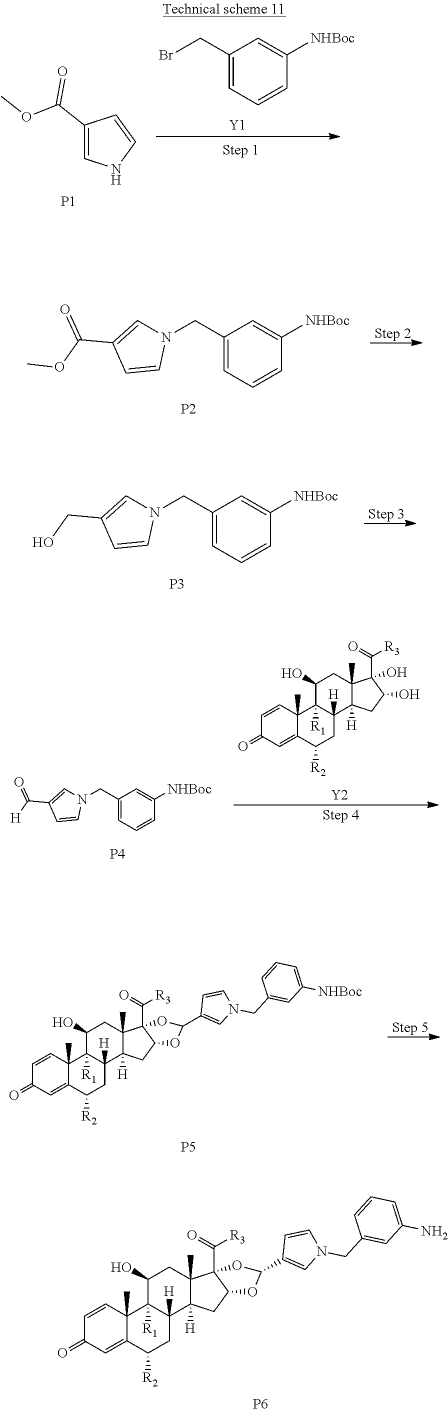

Step 1: reacting a compound of general formula (P1) with a compound of general formula (Y1) under optional basic conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5); and
Step 5: removing the protecting group tert-butyloxycarbonyl from the compound of general formula (P5) under optional acidic conditions, followed by separation by preparative HPLC, to obtain a compound of general formula (P6).

In the above steps,
R₁, R₂ and R₃ may be defined as R₁, R₂ and R₃ in any one of formula (II), formula (II-a1), formula (II-a2), formula (II-b1) or formula (II-b2) of the present application;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium borohydride, sodium cyanoborohydride, lithium aluminum hydride and sodium hydride;
reagents that provide oxidation conditions include, but are not limited to, Dess-Martin periodinane, hydrogen peroxide, sodium chlorite, sodium hypochlorite, and potassium perchlorate.

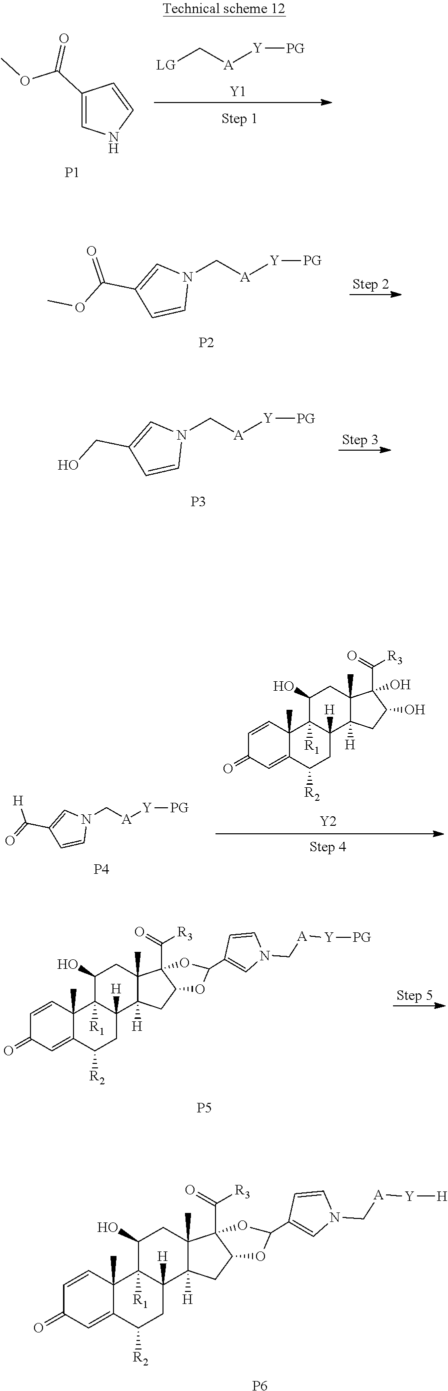

Step 1: reacting a compound of general formula (P1) with a compound of general formula (Y1) under optional acidic or basic conditions to obtain a compound of general formula (P2);
Step 2: reacting the compound of general formula (P2) under optional reducing conditions to obtain a compound of general formula (P3);
Step 3: reacting the compound of general formula (P3) under optional oxidation conditions to obtain a compound of general formula (P4);
Step 4: reacting the compound of general formula (P4) with a compound of general formula (Y2) under optional acidic or basic conditions to obtain a compound of general formula (P5);
and
Step 5: removing the protecting group PG from the compound of general formula (P5) under optional acidic or basic conditions, followed by separation by preparative HPLC, to obtain a compound of general formula (P6).

In the above steps,
PG may be a common hydroxy, amino, sulfhydryl or carboxy protecting group;
R₁, R₂ and R₃ may be defined as R₁, R₂ and R₃ in any formula (II) of the present application;
A and Y may be defined as A and Y in any of the formulae (II) of the present application; or A may be optionally substituted alkyl, optionally substituted heteroalkyl, optionally substituted aliphatic ring, optionally substituted aliphatic heterocyclic ring, optionally substituted aromatic ring, optionally substituted aromatic heterocyclic ring, or optionally substituted linear or branched chain structure;
or Y may be —O—, —NR— or —S—, wherein R may each independently be hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H or a C1-6 aliphatic group;
LG may be a common leaving group, including but not limited to, fluorine, chlorine, bromine, iodine, trifluoromethanesulfonyloxy, methanesulfonyloxy and p-toluenesulfonyloxy;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride;
reagents that provide reducing conditions include, but are not limited to, sodium borohydride, sodium cyanoborohydride, lithium aluminum hydride and sodium hydride;
reagents that provide oxidation conditions include, but are not limited to, Dess-Martin periodinane, hydrogen peroxide, sodium chlorite, sodium hypochlorite, and potassium perchlorate.

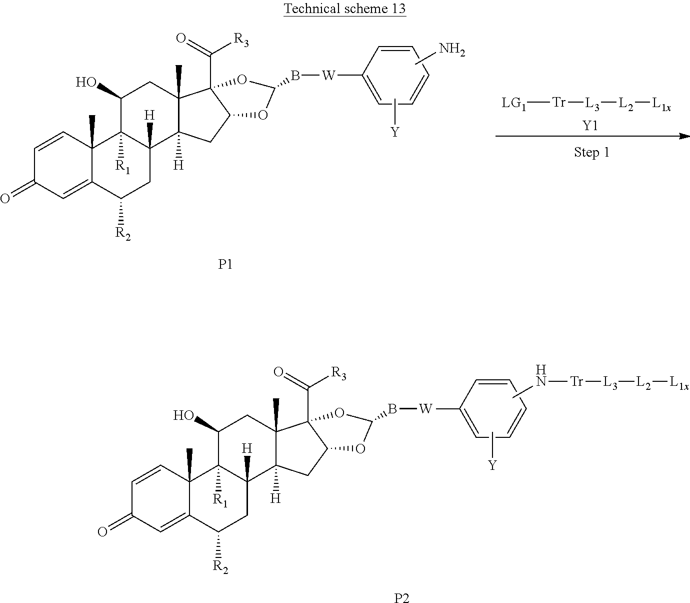

Step 1: reacting a compound of general formula (P1) with a compound of general formula (YT) under optional condensation conditions, followed by separation by preparative HPLC, to obtain a compound of general formula (P2).

In the above steps,
ring B is

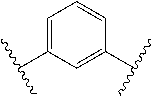 [SMILES: *c1cccc(*)c1]

or heteroaryl;
W is absent or W is any group;
Y is hydrogen, halogen, C₁-C₆ alkyl, C₁-C₆ haloalkyl, cyano, hydroxy, thiol, amino, alkylthio and alkoxy;
R₁, R₂ and R₃ may be defined as R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
LG₁ may be common hydroxy of activated ester or carboxy;
Tr is absent or Tr is any group;
L3 is selected from the polypeptide fragment L³ of the present application;
L2 is absent or selected from the linker fragment L² of the present application;
L1x is selected from the coupling unit L^{1x} of the present application; the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride.

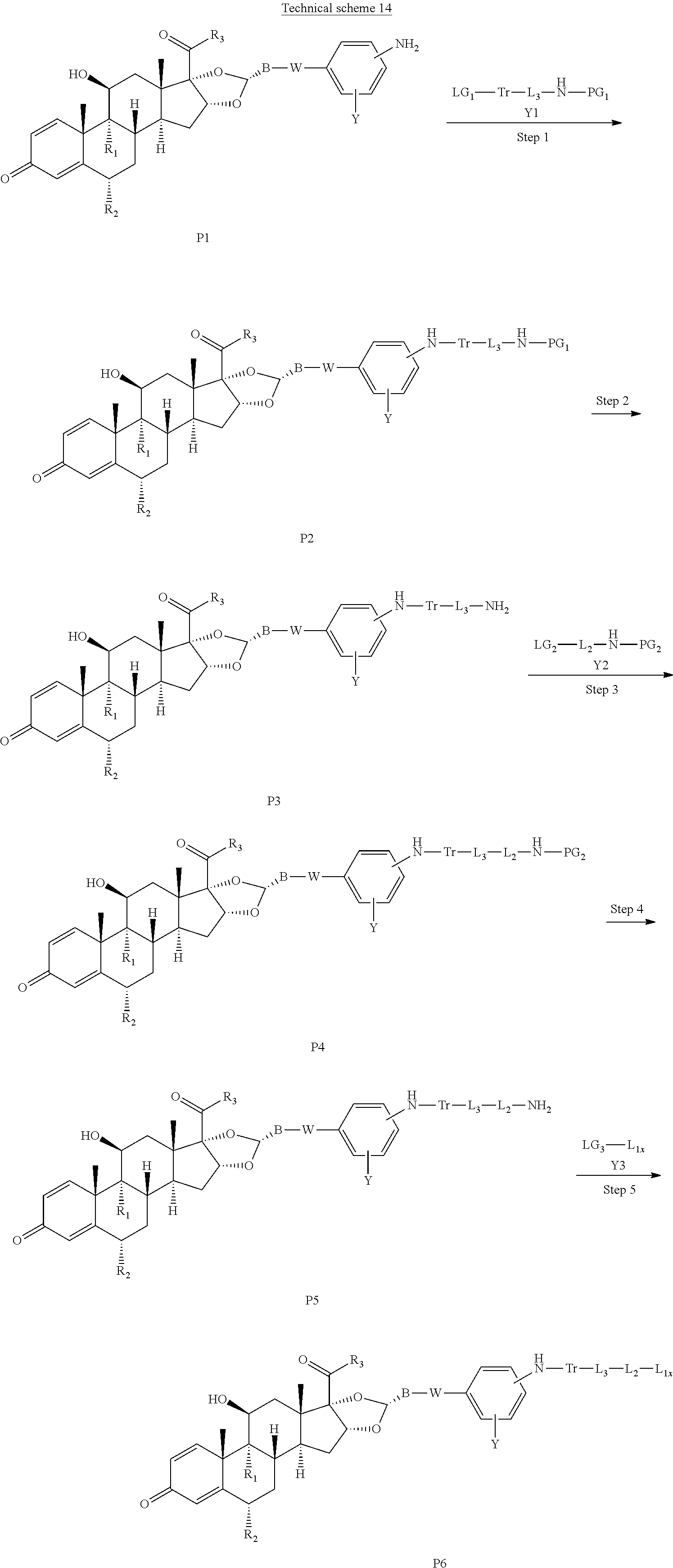

Step 1: reacting a compound of general formula (P1) with a compound of general formula (Y1) under optional condensation conditions to obtain a compound of general formula (P2);
Step 2: removing the protecting group PG₁ from the compound of general formula (P2) under optional acidic or basic conditions to obtain a compound of general formula (P3);
Step 3: reacting the compound of general formula (P3) with a compound of general formula (Y2) under optional condensation conditions to obtain a compound of general formula (P4);
Step 4: removing the protecting group PG₁ from the compound of general formula (P4) under optional acidic or basic conditions to obtain a compound of general formula (P5);
Step 5: reacting the compound of general formula (P5) with a compound of general formula (Y3) under optional condensation conditions, followed by separation by preparative HPLC, to obtain a compound of general formula (P6).

In the above steps,
ring B is

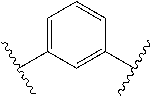 [SMILES: *c1cccc(*)c1]

or heteroaryl;
W is absent or W is any group;
Y is hydrogen, halogen, C₁-C₆ alkyl, C₁-C₆ haloalkyl, cyano, hydroxy, thiol, amino, alkylthio and alkoxy;
LG₁, LG₂ and LG₃ may be common hydroxy of activated ester or carboxy;
PG₁, PG₂ and PG₃ may be common amino protecting groups;
R₁, R₂ and R₃ may be defined as R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
Tr is absent or Tr is any group;
L3 is selected from the polypeptide fragment L³ of the present application;
L2 is absent or selected from the linker fragment L² of the present application;
L1x is selected from the coupling unit L^{1x} of the present application; the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

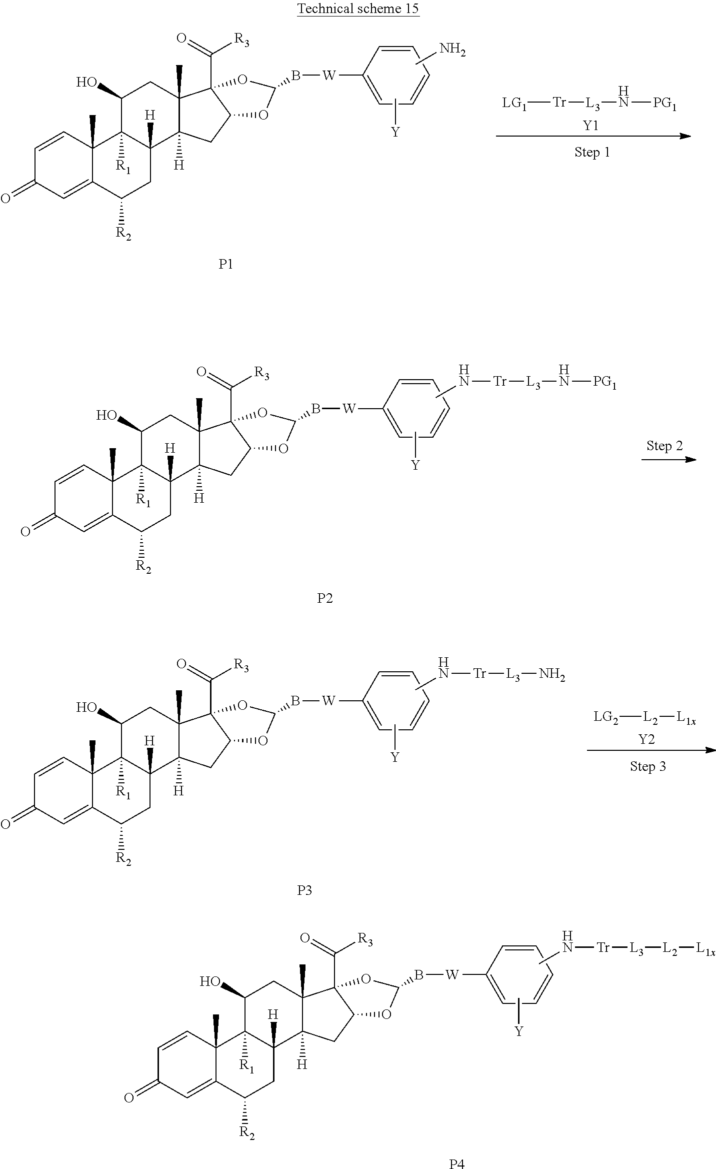

Step 1: reacting a compound of general formula (P1) with a compound of general formula (YT) under optional condensation conditions to obtain a compound of general formula (P2);
Step 2: removing the protecting group PG₁ from the compound of general formula (P2) under optional acidic or basic conditions to obtain a compound of general formula (P3);
Step 3: reacting the compound of general formula (P3) with a compound of general formula (Y3) under optional condensation conditions, followed by separation by preparative HPLC, to obtain a compound of general formula (P4).

In the above steps,
ring B is

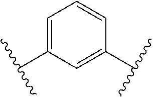 [SMILES: *c1cccc(*)c1]

or heteroaryl;
W is absent or W is any group;
Y is hydrogen, halogen, C₁-C₆ alkyl, C₁-C₆ haloalkyl, cyano, hydroxy, thiol, amino, alkylthio and alkoxy;
LG₁ and LG₂ may be common hydroxy of activated ester or carboxy;
PG₁ may be a common amino protecting group;
R₁, R₂ and R₃ may be defined as R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
Tr is absent or Tr is any group;
L3 is selected from the polypeptide fragment L³ of the present application;
L2 is absent or selected from the linker fragment L² of the present application;
L1x is selected from the coupling unit L^{1x} of the present application; the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

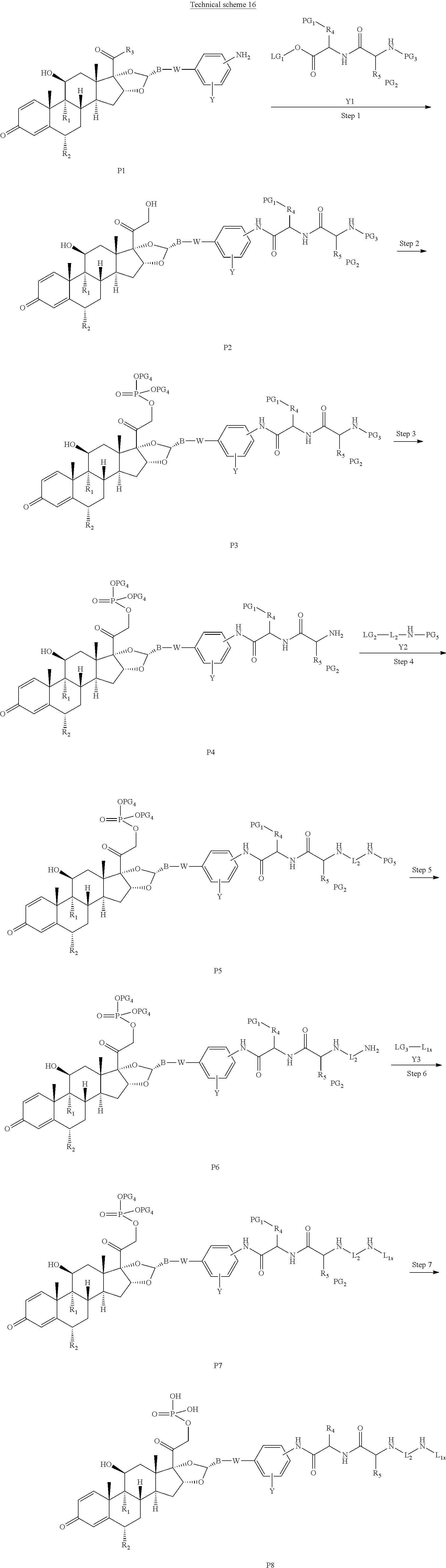

Step 1: reacting a compound of general formula (P1) with a compound of general formula (YT) under optional condensation conditions to obtain a compound of general formula (P2);
Step 2: adding a phosphate ester with a protecting group onto the compound of general formula (P2) under optional acidic or basic conditions to obtain a compound of general formula (P3);
Step 3: removing the protecting group PG₃ from the compound of general formula (P3) under optional acidic or basic conditions to obtain a compound of general formula (P4);
Step 4: reacting the compound of general formula (P4) with a compound of general formula (Y2) under optional condensation conditions to obtain a compound of general formula (P5);
Step 5: removing the protecting group PG₅ from the compound of general formula (P5) under optional acidic or basic conditions to obtain a compound of general formula (P6);
Step 6: reacting the compound of general formula (P6) with a compound of general formula (Y3) under optional condensation conditions to obtain a compound of general formula (P7); and
Step 7: removing the protecting group PG₁ from the compound of general formula (P7) under optional acidic or basic conditions, followed by separation by preparative HPLC, to obtain a compound of general formula (P8).

In the above steps,
ring B is

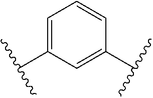 [SMILES: *c1cccc(*)c1]

or heteroaryl;
W is absent or W is any group;
Y is hydrogen, halogen, C₁-C₆ alkyl, C₁-C₆ haloalkyl, cyano, hydroxy, thiol, amino, alkylthio and alkoxy;
R₄ and R₅ are common amino acid residues;
LG₁, LG₂ and LG₃ may be common activated esters or hydrogen;
PG₁ and PG₂ may be absent or common amino protecting groups or hydroxy protecting groups or carboxyl protecting groups;
PG₃ and PG₅ may be common amino protecting groups;
PG₄ may be a common hydroxy protecting group;
R₁, R₂ and R₃ may be defined as R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
Tr is absent or Tr is any group;
L3 is selected from the polypeptide fragment L³ of the present application;
L2 is absent or selected from the linker fragment L² of the present application;
L1x is selected from the coupling unit L^{1x} of the present application;
conditions that provide the phosphate ester described above include, but are not limited to, diphenyl N,N′-diisopropylphosphoramidite/hydrogen peroxide, di-tert-butyl N,N-diisopropylphosphoramidite/hydrogen peroxide, bis[2-(trimethylsilyl)ethyl]diisopropylphosphoramidate/hydrogen peroxide, dibenzyl diethylphosphoramidite/hydrogen peroxide, dibenzyl N,N-dimethylphosphoramidite, phosphorus trichloride/benzyl alcohol/hydrogen peroxide, Pyrophosphoryl Chloride/benzyl alcohol and Pyrophosphoryl Chloride/tert-butanol;
the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

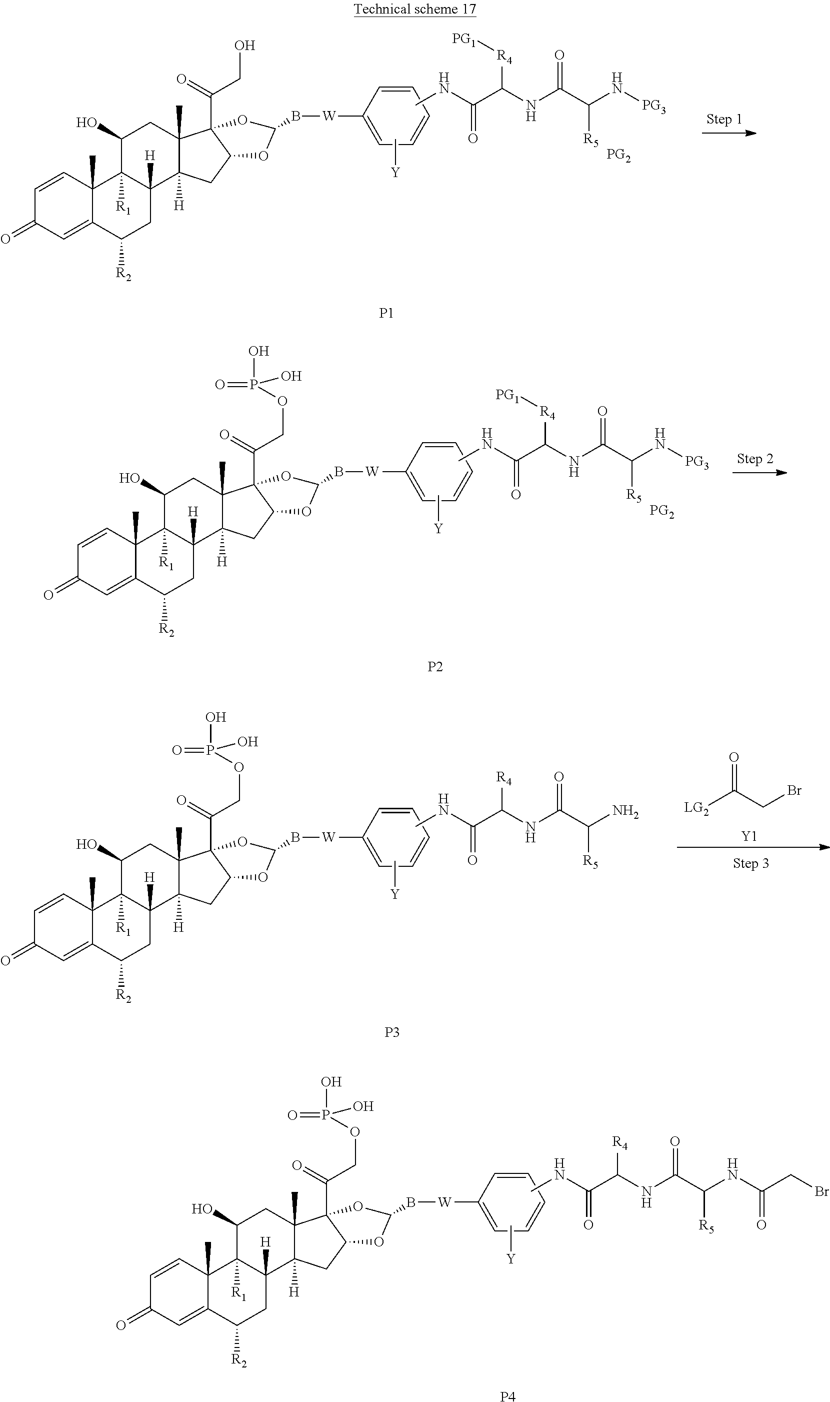

Step 1: introducing phosphoric acid into a compound of general formula (P1) under optional acidic or basic conditions to obtain a compound of general formula (P2);
Step 2: simultaneously or sequentially removing protecting groups PG₁, PG₂ and PG₃ from the compound of general formula (P2) under optional acidic or basic conditions to obtain a compound of general formula (P3); and
Step 3: reacting the compound of general formula (P3) with a compound of general formula (Y1) under optional condensation conditions, followed by separation by preparative HPLC, to obtain a compound of general formula (P4).

In the above steps,
ring B

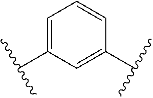 [SMILES: *c1cccc(*)c1]

or heteroaryl;
W is absent or W is any group;
Y is hydrogen, halogen, C₁-C₆ alkyl, C₁-C₆ haloalkyl, cyano, hydroxy, thiol, amino, alkylthio and alkoxy;
R₄ and R₅ are common amino acid residues;
PG₁ and PG₂ may be absent or common amino protecting groups or hydroxy protecting groups or carboxyl protecting groups;
PG₃ may be a common amino protecting group;
LG₂ may be a common activated ester or bromine;
R₁, R₂ and R₃ may be defined as R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
conditions that provide the phosphoric acid described above include, but are not limited to, Pyrophosphoryl Chloride, phosphorus oxychloride and phosphorus trichloride/hydrogen peroxide;
the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

Without being bound by any theory, the following examples are intended only to illustrate the compounds, preparation methods, use, etc., of the present application, and are not intended to limit the scope of the present application.

Without being bound by any theory, the following examples are intended only to illustrate the compounds, preparation methods, use, etc., of the present application, and are not intended to limit the scope of the present application.

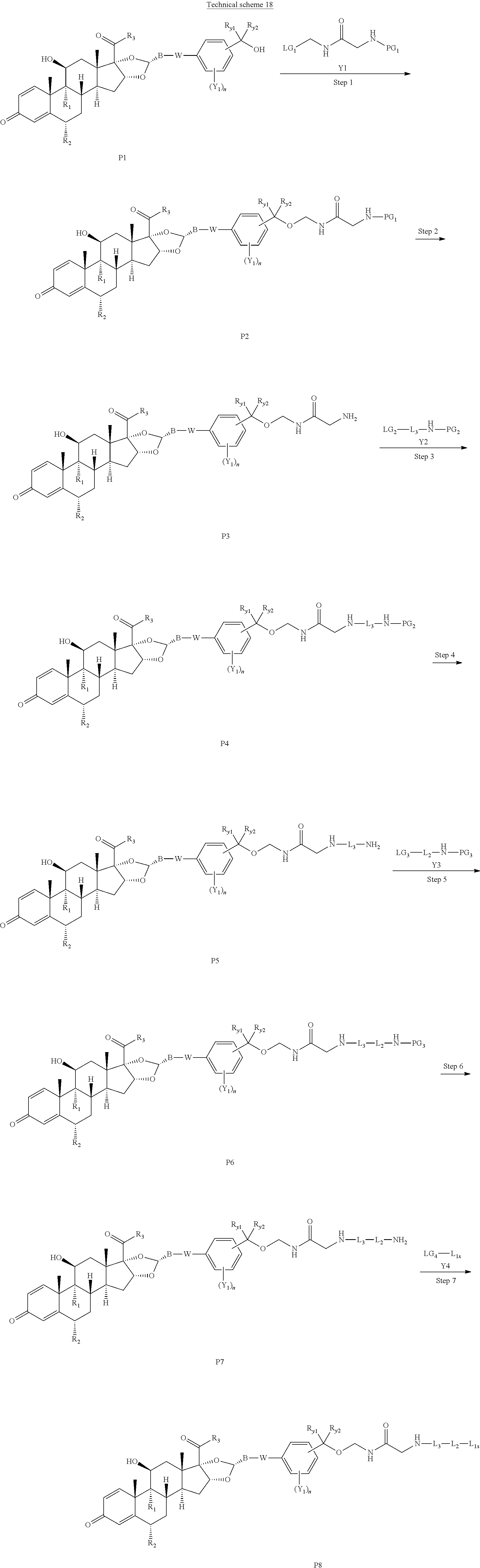

Step 1: reacting a compound of general formula (P1) with a compound of general formula (Y1) under optional condensation conditions to obtain a compound of general formula (P2);
Step 2: removing the protecting group PG1 from the compound of general formula (P2) under optional acidic or basic conditions to obtain a compound of general formula (P3);
Step 3: reacting the compound of general formula (P3) with a compound of general formula (Y2) under optional condensation conditions to obtain a compound of general formula (P4);
Step 4: removing the protecting group PG₂ from the compound of general formula (P4) under optional acidic or basic conditions to obtain a compound of general formula (P5);
Step 5: reacting the compound of general formula (P5) with a compound of general formula (Y3) under optional condensation conditions to obtain a compound of general formula (P6);
Step 6: removing the protecting group PG₃ from the compound of general formula (P6) under optional acidic or basic conditions to obtain a compound of general formula (P7); and
Step 7: reacting the compound of general formula (P7) with a compound of general formula (Y3) under optional condensation conditions, followed by separation by preparative HPLC, to obtain a compound of general formula (P8).

In the above steps,
ring B is optionally substituted aryl or heteroaryl;
W is absent or W is any group;
Y₁ is hydrogen, hydroxy, fluoro, chloro, methyl, hydroxy or methoxy;
R_{y1}, R_{y2}, n, R₁, R₂ and R₃ may be defined as R_{y1}, R_{y2}, n, R₁, R₂ and R₃ in any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application;
LG₁, LG₂, LG₃ and LG₄ may be common hydroxy of activated esters or carboxy;
PG₁, PG₂ and PG₃ may be common amino protecting groups;
Tr is absent or Tr is any group;
L3 is selected from the polypeptide fragment L³ of the present application;
L2 is absent or selected from the linker fragment L² of the present application;
L1x is selected from the coupling unit L^{1x} of the present application; the condensing agent may be selected from 4-(4,6-dimethoxy-1,3,5-triazin-2-yl)-4-methylmorpholinium chloride, 1-hydroxybenzotriazole and 1-(3-dimethylaminopropyl)-3-ethylcarbodiimide hydrochloride, N,N′-dicyclohexylcarbodiimide, N,N′-diisopropylcarbodiimide, O-benzotriazol-N,N,N′,N′-tetramethyluronium tetrafluoroborate, 1-hydroxybenzotriazole, 1-hydroxy-7-azobenzotriazol, O-benzotriazol-N,N,N′,N′-tetramethyluronium hexafluorophosphate, 2-(7-azobenzotriazol)-N,N,N′,N′-tetramethyluronium hexafluorophosphate, benzotriazol-1-yloxytris(dimethylamino)phosphonium hexafluorophosphate or benzotriazol-1-yl-oxytripyrrolidinophosphonium hexafluorophosphate, preferably 4-(4,6-dimethoxy-1,3,5-triazin-2-yl)-4-methylmorpholinium chloride, or 1-hydroxybenzotriazole and 1-(3-dimethylaminopropyl)-3-ethylcarbodiimide hydrochloride;
reagents that provide basic conditions include organic bases and inorganic bases, wherein the organic bases include, but are not limited to, triethylamine, diethylamine, N-methylmorpholine, pyridine, piperidine, N,N′-diisopropylethylamine, n-butyllithium, lithium diisopropylamide, potassium acetate, sodium tert-butoxide, potassium tert-butoxide, and the like, and the inorganic bases include, but are not limited to, sodium hydride, potassium carbonate, sodium carbonate, cesium carbonate, sodium hydroxide, lithium hydroxide, and the like;
reagents that provide acidic conditions include protic acids and Lewis acids, wherein the protic acids include, but are not limited to, hydrochloric acid, sulfuric acid, nitric acid, nitrous acid, sulfurous acid, phosphoric acid, phosphorous acid, formic acid, acetic acid, propionic acid, butyric acid, citric acid, benzoic acid, p-toluenesulfonic acid, p-nitrobenzoic acid, methanesulfonic acid, trifluoromethanesulfonic acid and trifluoroacetic acid, and the Lewis acids include, but are not limited to, boron trifluoride, zinc chloride, magnesium chloride, aluminum chloride, stannic chloride and ferric chloride.

Without being bound by any theory, the following examples are intended only to illustrate the compounds, preparation methods, use, etc., of the present application, and are not intended to limit the scope of the present application.

Without being bound by any theory, the following examples are intended only to illustrate the compounds, preparation methods, use, etc., of the present application, and are not intended to limit the scope of the present application.

### EXAMPLES

the structure of the compounds is determined by nuclear magnetic resonance (NMR) or mass spectrometry (MS). NMR is performed using a Quan tum-I NMR spectrometer with deuterated dimethyl sulfoxide (DMSO-D), deuterated chloroform (CDCl3), and deuterated methanol (CD30D) as solvents, and tetramethylsilane (TMS) as an internal standard, and chemical shifts are given in units of 10_6(ppm).

MS is performed using an Angilent 6230 ESI-TOF mass spectrometer (manufacturer: Agilent, type c: 6230).

UPLC is performed using a Waters AcquityUPLCSQD liquid chromatograph-mass spectrometer (Poroshell 120 EC-C18, 2.1 mm×50 mm, 1.9 μm column).

HPLC is performed using an Agilent 1260 high-performance liquid chromatograph (TOSOH G3000 SW SEC column).

UV is performed using a Thermo Nanodrop 2000 spectrophotometer.

Enzyme-linked immunoassays are performed using an EnVision microplate reader (PerkinElmer).

Yantai Yellow Sea HSGF254 or Qingdao GF254 silica gel plate is adopted as a thin layer chromatography (TLC) silica gel plate. The specification adopted by the TLC is 0.15-0.2 mm, and the specification adopted by the thin layer chromatography for the separation and purification of products is 0.4-0.5 mm.

Yantai Yellow Sea silica gel of 200-300 mesh is generally utilized as a carrier in column chromatography.

Known starting materials of the present application can be synthesized using or according to methods known in the art, or can be purchased from companies such as ABCR GmbH & Co. KG, Acros Organnics, Aldrich Chemical Company, Accela ChemBio Inc., and Darui Chemicals.

In the examples, all reactions are carried out under an argon atmosphere or a nitrogen atmosphere unless otherwise stated.

The argon atmosphere or nitrogen atmosphere means that the reaction flask is connected to a balloon containing about 1 L of argon or nitrogen.

The hydrogen atmosphere means that the reaction flask is connected to a balloon containing about 1 L of hydrogen.

In the examples, the solution in the reaction is an aqueous solution unless otherwise stated.

In the examples, the reaction temperature is room temperature unless otherwise stated. The room temperature is the optimum reaction temperature, which ranges from 20° C. to 30° C. The system of eluents for column chromatography and the system of developing agents for thin layer chromatography used for purifying compounds include: A: dichloromethane and isopropanol system, B: dichloromethane and methanol system, and C: petroleum ether and ethyl acetate system. The volume ratio of solvents is regulated according to different polarities of the compound, and can also be regulated by adding a small amount of triethylamine and acidic or alkaline reagent.

Some of the compounds of the present disclosure are characterized by TOF-LC/MS. TOF-LC/MS analysis is performed using an Agilent 6230 time-of-flight mass spectrometer and an Agilent 1290-Infinity ultra-high performance liquid chromatograph.

### Example 1

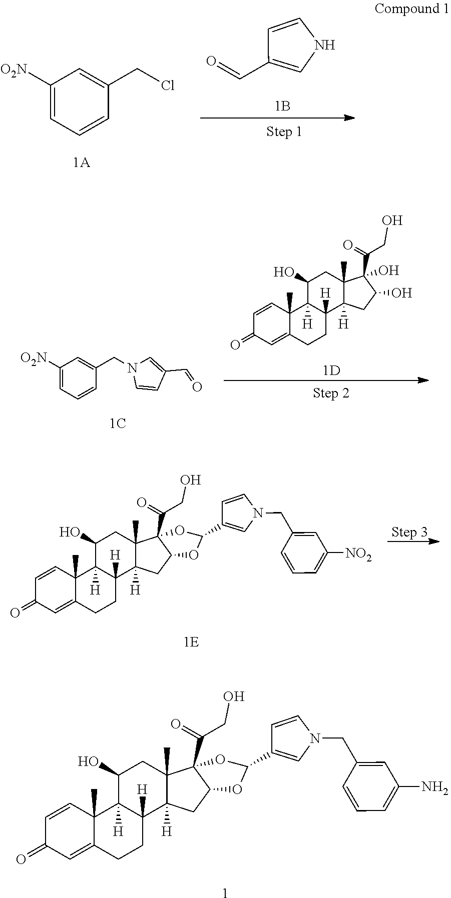

Step 1 Compound 1A (200 mg, 1.16 mmol, 1.0 eq), 1B (112 mg, 1.18 mmol, 1.0 eq) and K₂CO₃ (330 mg, 2.39 mmol, 2.0 eq) were added to a 50 mL single-neck flask, DMF (10 mL) was added, and the reaction solution was reacted at 100° C. for 24 h under nitrogen atmosphere. Ethyl acetate (100 mL) was added to the reaction solution, and the mixed solution was washed with saturated brine (100 mL×5), dried over anhydrous sodium sulfate, filtered, concentrated by rotary evaporation and purified by column chromatography (PE/EA=100/1 to 100/50) to give a light yellow solid 1C (160 mg, yield: 60%). MS-ESI: m/z 231.1 [M+H]^{+.}

Step 2 Compound 1C (220 mg, 0.96 mmol, 1.0 eq), 1D (362 mg, 0.96 mmol, 1.0 eq) and MgSO₄ (360 mg, 2.99 mmol, 3.0 eq) were added to a 50 mL three-necked flask, and anhydrous acetonitrile (10 mL) was added. Under nitrogen atmosphere, methanesulfonic acid (280 mg, 2.91 mmol, 3.0 eq) was added at 0° C. and reacted for 2 h. The reaction solution was filtered, concentrated by rotary evaporation and purified by column chromatography (DCM/MeOH=100/1 to 100/5) to give a light yellow solid 1E (100 mg, yield: 18%). MS-ESI: m/z 589.3 [M+H]^{+.}

Step 3 Under nitrogen atmosphere, compound 1E (100 mg, 0.17 mmol, 1.0 eq) and iron powder (93 mg, 1.67 mmol, 9.8 eq) were added to ethanol (3 mL). Ammonium chloride (89 mg, 1.66 mmol, 9.8 eq) was dissolved in water (1 mL), and the resulting solution was added dropwise to the system. The system was heated to 60° C. and stirred for 3 h. After the starting materials were completely consumed and a product was generated as detected by LCMS, the reaction solution was filtered through celite, concentrated by rotary evaporation under reduced pressure and purified by preparative chromatography to give a white powdery solid product 1 (35 mg, yield: 37%).

MS-ESI: m/z 559.3 [M+H]^{+.}

¹H NMR (400 MHz, Chloroform-d) δ 7.23 (d, J=10.1 Hz, 1H), 7.08 (t, J=7.8 Hz, 1H), 6.76-6.72 (m, 1H), 6.65 (t, J=2.5 Hz, 1H), 6.58 (dd, J=8.0, 2.2 Hz, 1H), 6.52 (d, J=7.6 Hz, 1H), 6.39 (brs, 1H), 6.29 (dd, J=10.1, 1.9 Hz, 1H), 6.24-6.19 (m, 1H), 6.07-6.02 (m, 1H), 5.46 (s, 1H), 5.00 (d, J=5.4 Hz, 1H), 4.91 (s, 2H), 4.62 (d, J=20.2 Hz, 1H), 4.49 (brs, 1H), 4.28 (d, J=20.2 Hz, 1H), 3.66 (brs, 1H), 3.03 (brs, 1H), 2.58 (td, J=13.6, 5.5 Hz, 1H), 2.40-2.31 (m, 1H), 2.26-2.06 (m, 3H), 2.01 (s, 1H), 1.91 (dd, J=12.6, 5.5 Hz, 1H), 1.87-1.77 (m, 1H), 1.69 (dd, J=12.8, 5.5 Hz, 2H), 1.65-1.58 (m, 3H), 1.45 (s, 3H), 1.30 (s, 1H), 1.25 (s, 1H), 1.19-1.13 (m, 1H), 1.11 (dd, J=11.2, 3.5 Hz, 1H), 0.95 (s, 3H).

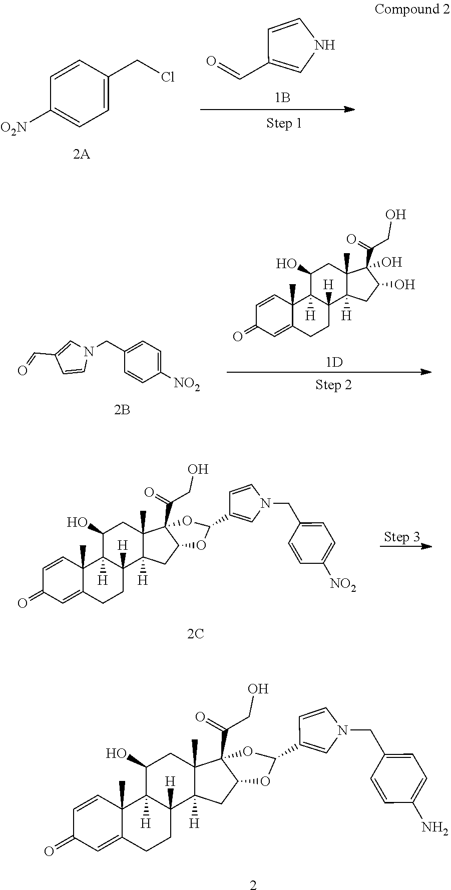

Step 1 Compound 2A (900 mg, 5.2 mmol, 1.0 eq), compound 1B (500 mg, 5.2 mmol, 1.0 eq) and K₂CO₃ (1.4 g, 10.4 mmol, 2 eq) were added to a 50 mL single-neck flask, DMF (10 mL) was added, and the reaction solution was reacted at 100° C. for 2 h under N₂ atmosphere. EA (100 mL) was added to the reaction solution, and the mixed solution was washed with saturated brine (100 mL×5), dried over anhydrous sodium sulfate, filtered, concentrated by rotary evaporation and purified by column chromatography (PE/EA=100/1 to 100/50) to give alight yellow solid 2B (900 mg, yield: 74%). MS-ESI: m/z 231.2 [M+H]^{+.}

Step 2 Compound 2B (900 mg, 3.9 mmol, 1.0 eq), compound 1D (1.47 g, 3.9 mmol, 1.0 eq) and MgSO₄ (1.41 g, 11.7 mmol, 3.0 eq) were added to a 50 mL three-necked flask, and anhydrous acetonitrile (10 mL) was added. Under N₂ atmosphere, methanesulfonic acid (1.12 g, 11.7 mmol, 3.0 eq) was added at 0° C. and reacted for 2 h. The reaction solution was filtered, concentrated by rotary evaporation and purified by column chromatography (DCM/MeOH=100/1 to 100/5) to give a light yellow solid 2C (430 mg, yield: 19%). MS-ESI: m/z 589.3 [M+H]^{+.}

Step 3 Compound 2C (430 mg, 0.73 mmol, 1.0 eq) and iron powder (409 mg, 7.3 mmol, 10 eq) were added to ethanol (10 mL). Ammonium chloride (391 mg, 7.3 mmol, 10 eq) was dissolved in water (2 mL), and the resulting solution was added dropwise to the system. Under nitrogen atmosphere, the system was heated to 60° C. and stirred for 1 h. After the starting materials were completely consumed and a product was generated as detected by LCMS, the reaction solution was filtered through celite, concentrated by rotary evaporation under reduced pressure and purified by preparative chromatography to give a white powdery solid product 2 (65 mg, yield: 16%).

MS-ESI: m/z 559.4 [M+H]^{+.}

¹H NMR (400 MHz, DMSO-d₆) δ 7.32 (d, J=10.1 Hz, 1H), 6.94-6.88 (m, 2H), 6.87 (t, J=1.9 Hz, 1H), 6.68 (t, J=2.5 Hz, 1H), 6.51-6.43 (m, 2H), 6.17 (dd, J=10.1, 1.9 Hz, 1H), 6.02 (dd, J=2.7, 1.7 Hz, 1H), 5.98-5.91 (m, 1H), 5.34 (s, 1H), 5.09-4.99 (m, 3H), 4.83-4.74 (m, 4H), 4.43 (dd, J=19.5, 6.3 Hz, 1H), 4.32-4.25 (m, 1H), 4.13 (dd, J=19.4, 5.8 Hz, 1H), 2.59-2.53 (m, 1H), 2.36-2.28 (m, 1H), 2.13-1.98 (m, 2H), 1.81-1.61 (m, 4H), 1.57 (dd, J=14.3, 5.4 Hz, 1H), 1.39 (s, 3H), 1.06-0.92 (m, 2H), 0.83 (s, 3H).

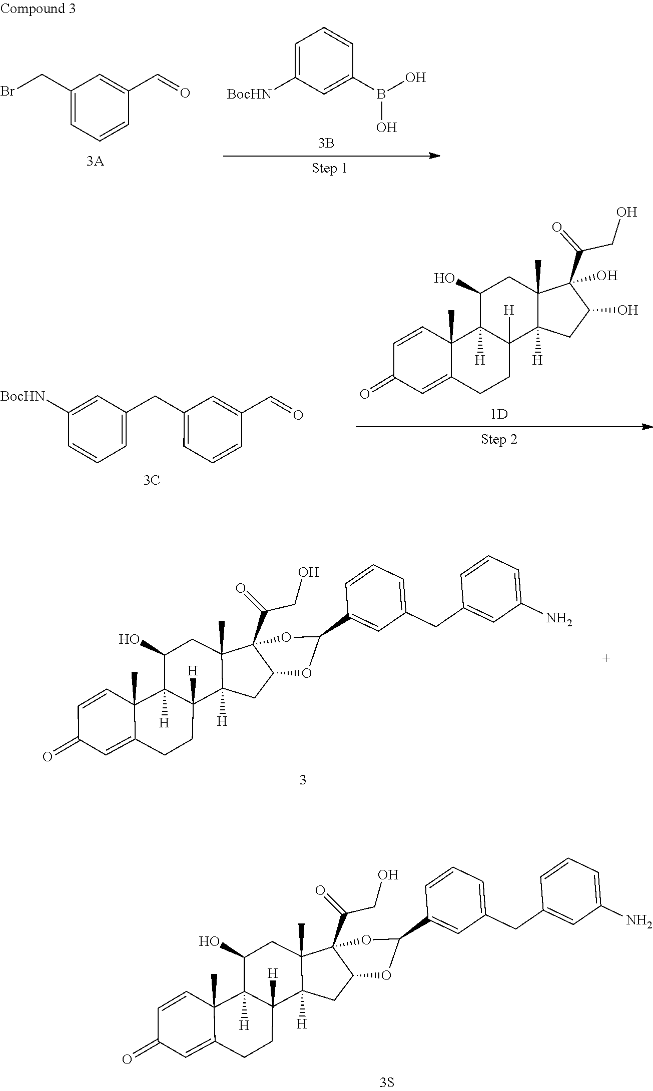

Step 1 Compound 3A (800 mg, 4.02 mmol, 1.0 eq), compound 3B (950 mg, 4.01 mmol, 1.0 eq) and K₂CO₃ (1.66 g, 12.03 mmol, 3.0 eq) were added to a three-necked flask (100 mL), and tetrahydrofuran (40 mL) was added for dissolution. Pd(dppf)Cl₂ (588 mg, 0.80 mmol, 0.2 eq) was added, and the reaction solution was purged three times with nitrogen, heated to 80° C. and stirred for 20 h. After the reaction was completed as detected by LCMS, the reaction solution was filtered. The mother liquor was extracted with dichloromethane (100 mL) and water (100 mL). The organic phase was dried, concentrated by rotary evaporation, mixed with silica gel and purified by column chromatography (PE/EA=9/1) to give a white solid 3C (800 mg, yield: 64%). MS-ESI: m/z 334.2 [M+Na]^{+.}

Step 2 Compound 3C (227 mg, 0.73 mmol, 1.0 eq), compound 1D (274 mg, 0.73 mmol, 1.0 eq) and MgSO₄ (175 mg, 1.45 mmol, 2.0 eq) were added to a 50 mL three-necked flask, and acetonitrile (10 mL) was added. Under nitrogen atmosphere, trifluoromethanesulfonic acid (328 mg, 2.19 mmol, 3.0 eq) was added at 0° C. and reacted for 2 h. After the reaction was completed as detected by LCMS, the reaction solution was filtered. Water (50 mL) and dichloromethane (50 mL) were added to the mother liquor for extraction and separation. The mixed solution was washed with saturated brine, and the organic phase was dried over anhydrous sodium sulfate, concentrated by rotary evaporation, purified by preparative chromatography and lyophilized to give a white solid powder 3 (72 mg, yield: 17%) and a white solid powder 3S (100 mg, yield: 24%).

### Compound 3

MS-ESI: m/z 570.6 [M+H]^{+.}

¹H NMR (400 MHz, DMSO-d₆) δ 7.36-7.25 (m, 5H), 7.12 (t, J=8.1 Hz, 1H), 6.92 (d, J=7.6 Hz, 1H), 6.85-6.79 (m, 2H), 6.20 (dd, J=10.2, 1.8 Hz, 1H), 5.99 (s, 1H), 5.39 (s, 1H), 4.91 (d, J=3.9 Hz, 1H), 4.81 (brs, 1H), 4.49 (d, J=19.5 Hz, 1H), 4.32-4.25 (m, 1H), 4.17 (d, J=19.5 Hz, 1H), 3.90 (s, 2H), 3.69 (brs, 1H), 2.61-2.53 (m, 1H), 2.38-2.30 (m, 1H), 2.18-1.94 (m, 2H), 1.80-1.55 (m, 5H), 1.40 (s, 3H), 1.05-0.90 (m, 2H), 0.85 (s, 3H).

### Compound 3S

MS-ESI: m/z 570.6 [M+H]^{+.}

¹H NMR (400 MHz, DMSO-d₆) δ 7.35-7.27 (m, 2H), 7.27-7.20 (m, 2H), 7.14 (s, 1H), 7.10 (d, J=7.4 Hz, 1H), 6.96 (d, J=7.8 Hz, 1H), 6.92-6.85 (m, 1H), 6.83 (s, 1H), 6.17 (dd, J=10.1, 1.9 Hz, 1H), 6.06 (s, 1H), 5.94 (s, 1H), 5.28 (d, J=6.9 Hz, 1H), 4.80 (brs, 1H), 4.35-4.28 (m, 1H), 4.25 (d, J=19.2 Hz, 1H), 4.02 (d, J=19.2 Hz, 1H), 3.90 (s, 2H), 2.60-2.54 (m, 1H), 2.36-2.27 (m, 1H), 2.11-1.96 (m, 2H), 1.89-1.67 (m, 5H), 1.39 (s, 3H), 1.26-1.11 (m, 1H), 1.04 (dd, J=11.2, 3.4 Hz, 1H), 0.88 (s, 3H).

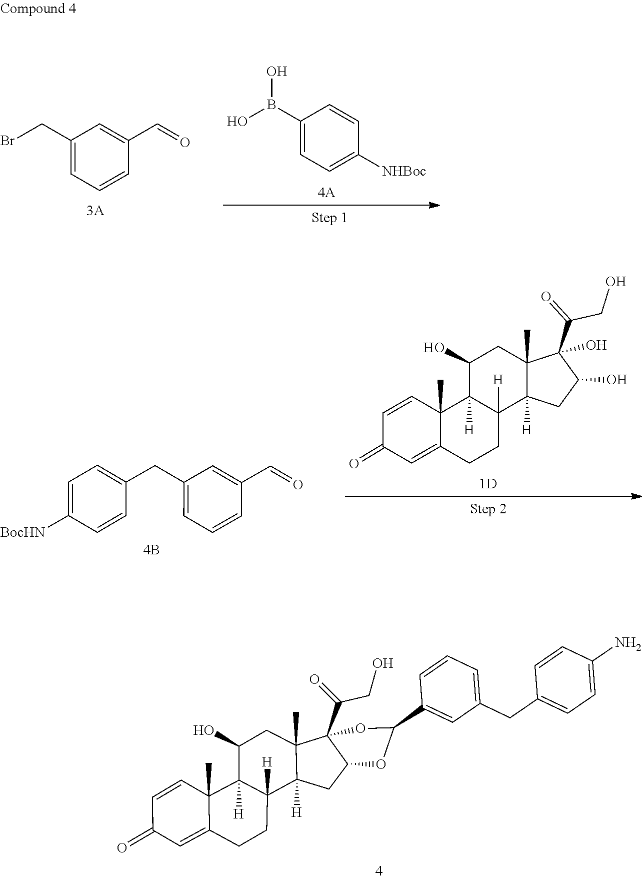

Step 1 Compound 3A (2.5 g, 12.56 mmol, 1.0 eq), compound 4A (2.98 g, 12.57 mmol, 1.0 eq) and K₂CO₃ (5.2 g, 37.63 mmol, 3.0 eq) were added to a three-necked flask (100 mL), and tetrahydrofuran (25 mL) was added for dissolution. Pd(dppf)Cl₂ (1.8 g, 2.46 mmol, 0.2 eq) was added, and the reaction solution was purged three times with nitrogen, heated to 80° C. and stirred for 20 h. After the reaction was completed as detected by TLC (PE/EA=5/1), the reaction solution was filtered. The mother liquor was extracted with dichloromethane (100 mL) and water (100 mL). The organic phase was dried, concentrated by rotary evaporation, mixed with silica gel and purified by column chromatography (PE/EA=9/1) to give a white solid 4B (2.5 g, yield: 64%). MS-ESI: m/z 334.1 [M+Na]^{+.}

Step 2 Compound 4B (227 mg, 0.73 mmol, 1.0 eq), compound 1D (274 mg, 0.73 mmol, 1.0 eq) and MgSO₄ (175 mg, 1.45 mmol, 2.0 eq) were added to a 50 mL three-necked flask, and acetonitrile (10 mL) was added. Under nitrogen atmosphere, trifluoromethanesulfonic acid (328 mg, 2.19 mmol, 3.0 eq) was added at 0° C. and reacted for 2 h. After the reaction was completed as detected by TLC (PE/EA=1/1), the reaction solution was filtered. Water (50 mL) and dichloromethane (50 mL) were added to the mother liquor for extraction and separation. The mixed solution was washed with saturated brine, and the organic phase was dried over anhydrous sodium sulfate, concentrated by rotary evaporation, purified by preparative chromatography and lyophilized to give a white solid powder 4 (320 mg, yield: 77%).

MS-ESI: m/z 570.3 [M+Na]^{+.}

¹H NMR (400 MHz, DMSO-d₆) δ 7.34 (d, J=10.0 Hz, 1H), 7.31-7.22 (m, 4H), 7.13 (d, J=8.1 Hz, 2H), 6.93 (d, J=7.9 Hz, 2H), 6.20 (dd, J=10.1, 1.9 Hz, 1H), 5.98 (s, 1H), 5.40 (s, 1H), 4.91 (d, J=4.5 Hz, 1H), 4.82 (brs, 1H), 4.49 (d, J=19.5 Hz, 1H), 4.31 (brs, 1H), 4.17 (d, J=19.5 Hz, 1H), 3.86 (s, 2H), 2.61-2.53 (m, 1H), 2.39-2.31 (m, 1H), 2.19-2.08 (m, 1H), 2.08-2.00 (m, 1H), 1.81-1.57 (m, 5H), 1.40 (s, 3H), 1.09-0.93 (m, 2H), 0.86 (s, 3H).

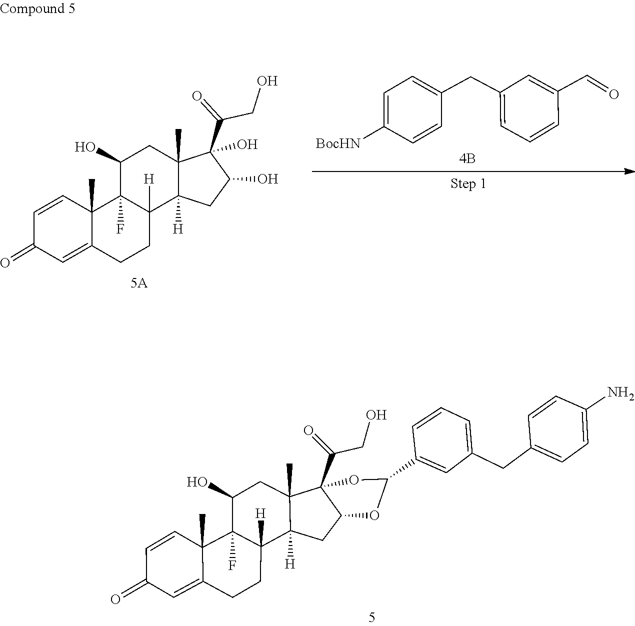

Step 1 Compound 4B (2 g, 6.42 mmol), compound 5A (2.5 g, 6.34 mmol) and MgSO₄ (2.4 g, 19.9 mmol) were dissolved in MeCN (30 mL). Under nitrogen atmosphere, the mixed solution was cooled to 0° C., and a solution of trifluoromethanesulfonic acid (1.93 g, 12.86 mmol) in MeCN (2 mL) was added dropwise. After addition, the mixed solution was reacted at room temperature for 17 h. After the starting materials were completely consumed as detected by LCMS, the reaction solution was filtered, concentrated by rotary evaporation, mixed with silica gel and purified by column chromatography (dichloromethane/MeOH=10/1) to give a white solid 5 (1.2 g, yield: 32%).

MS-ESI: m/z 588.3 [M+H]^{+.}

¹H NMR (400 MHz, DMSO-d₆) δ 7.36-7.19 (m, 5H), 7.15 (d, J=8.3 Hz, 2H), 6.97 (d, J=8.1 Hz, 2H), 6.26 (dd, J=10.3, 1.9 Hz, 1H), 6.10 (s, 1H), 5.52-5.41 (m, 2H), 4.91 (s, 1H), 4.50 (d, J=19.5 Hz, 1H), 4.27-4.12 (m, 2H), 3.89-3.79 (m, 2H), 2.73-2.56 (m, 2H), 2.43-2.35 (m, 1H), 2.25-2.03 (m, 2H), 1.90-1.81 (m, 1H), 1.74-1.55 (m, 3H), 1.51 (s, 3H), 1.45-1.31 (m, 1H), 0.88 (s, 3H).

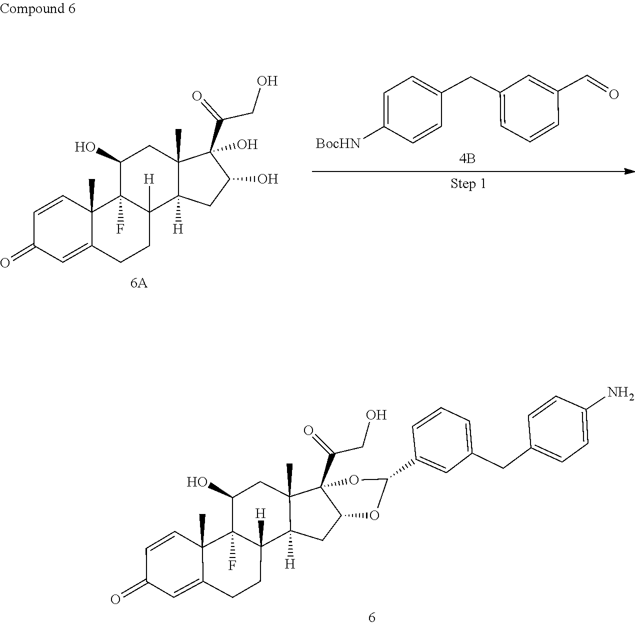

Step 1 Compound 4B (100 mg, 0.24 mmol, 1.0 eq), compound 6A (75 mg, 0.24 mmol, 1.0 eq) and MgSO₄ (58 mg, 0.48 mmol, 2.0 eq) were added to a 25 mL three-necked flask, and acetonitrile (5 mL) was added for dissolution. Under nitrogen atmosphere, the mixed solution was stirred at 80° C. for 10 min and then trifluoromethanesulfonic acid (109 mg, 0.73 mmol, 3.0 eq) was added. After 2 h, the reaction was completed as detected by TLC (EA), and the reaction solution was filtered. The mother liquor was concentrated by rotary evaporation and then purified by preparative chromatography to give an off-white solid 6 (62 mg, yield: 42%). MS-ESI: m/z 606.1 [M+H]^{+.}

¹H NMR (400 MHz, DMSO-d₆) δ 7.36-7.21 (m, 5H), 7.14 (d, J=8.0 Hz, 2H), 6.95 (d, J=7.9 Hz, 2H), 6.32 (dd, J=10.1, 1.8 Hz, 1H), 6.18 (s, 1H), 5.77-5.53 (m, 2H), 5.46 (s, 1H), 4.96-4.91 (m, 1H), 4.50 (d, J=19.5 Hz, 1H), 4.26-4.15 (m, 2H), 3.90-3.76 (m, 3H), 2.76-2.57 (m, 1H), 2.37-2.20 (m, 2H), 2.10-2.02 (m, 1H), 1.74-1.63 (m, 3H), 1.57-1.43 (m, 4H), 0.87 (s, 3H).

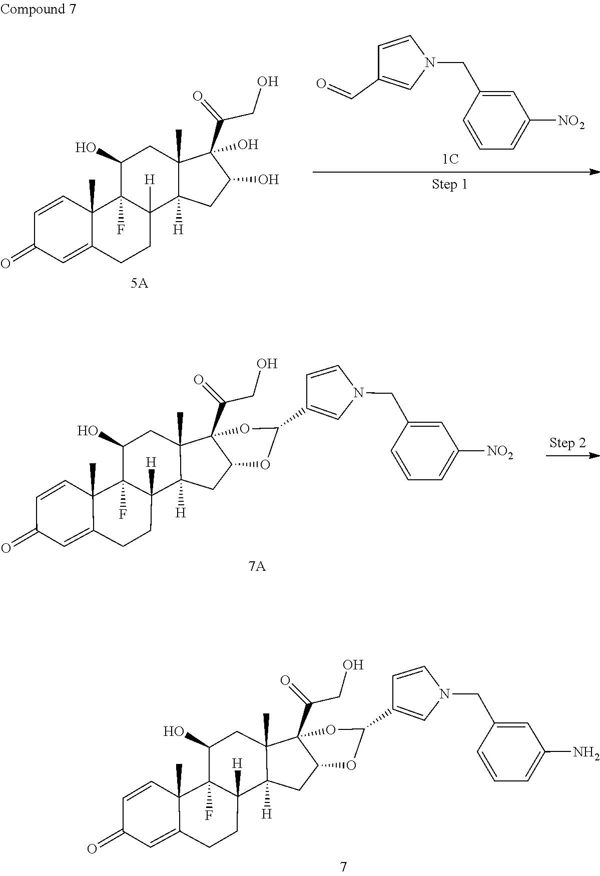

Step 1 compound 1C (2 g, 8.69 mmol, 1.0 eq) and compound 5A(3.4 g, 8.69 mmol, 1.0 eq) were added to ACN (50 mL), and then MgSO₄ (5.2 g, 43.4 mmol, 5.0 eq) was added. Methanesulfonic acid (2.5 g, 26.1 mmol, 3.0 eq, dissolved in 2 mL ACN) was added at 0° C., and the mixed solution was reacted for 20 min. After a product was produced as detected by LCMS, NaHCO₃ solid (3 g) was added to the reaction solution, then water (30 mL) was added, and the mixed solution was stirred for 10 min and filtered. The filter cake was washed with DCM/MeOH (10/l, 100 mL), and the aqueous phase was removed by liquid separation. The organic phase was dried, mixed with silica gel and purified by column chromatography (PE/EA=1/3) to give a yellow solid 7A (650 mg, yield: 12%). MS-ESI: m/z 607.3 [M+H]^{+.}

Step 2 Compound 7A (640 mg, 1.06 mmol, 1.0 eq) was added to EtOH/H₂O (8 mL/2 mL), and then NH₄Cl (562 mg, 10.6 mmol, 10.0 eq) and iron powder (594 mg, 10.6 mmol, 10.0 eq) were added. Under N₂ atmosphere, the mixed solution was heated to 60° C. and reacted for 1 h. After the reaction was completed as detected by TLC (PE/EA=1/2), the reaction solution was cooled to room temperature, and then Na₂SO₄ (5 g) was added. The mixed solution was filtered, the filter cake was washed with DCM/MeOH (10/l, 100 mL), and the filtrate was concentrated by rotary evaporation, mixed with silica gel and purified by column chromatography (DCM/MeOH=10/1) to give a yellow solid 7 (385 mg, yield: 63%).

MS-ESI: m/z 577.3 [M+H]^{+.}

¹H NMR (400 MHz, DMSO-d₆) δ 7.29 (d, J=10.1 Hz, 1H), 6.98-6.89 (m, 1H), 6.89-6.82 (m, 1H), 6.75-6.67 (m, 1H), 6.47-6.40 (m, 1H), 6.38-6.19 (m, 3H), 6.07-5.97 (m, 2H), 5.45-5.36 (m, 2H), 5.11-4.98 (m, 3H), 4.91-4.77 (m, 3H), 4.45 (dd, J=19.4, 6.2 Hz, 1H), 4.24-4.09 (m, 2H), 2.71-2.58 (m, 1H), 2.58-2.52 (m, 1H), 2.49-2.41 (m, 1H), 2.40-2.29 (m, 1H), 2.23-2.11 (m, 1H), 2.11-2.01 (m, 1H), 1.89-1.79 (m, 1H), 1.69-1.55 (m, 3H), 1.50 (s, 3H), 1.44-1.31 (m, 1H), 0.84 (s, 3H).

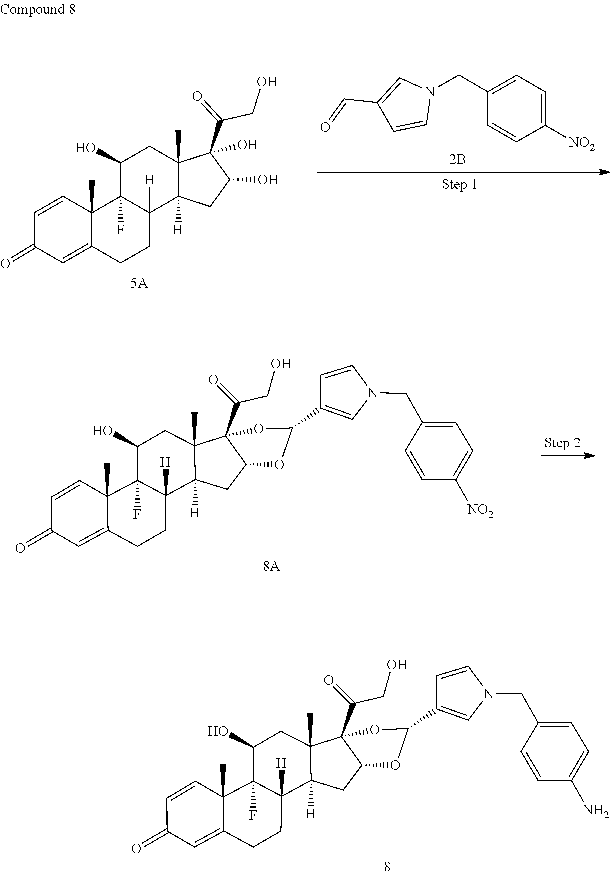

Step 1 Compound 2B (4 g, 17.37 mmol) and compound 5A (6.85 g, 17.37 mmol) were dissolved in MeCN (40 mL), and MgSO₄ (6.26 g, 52.11 mmol) was added. Under nitrogen atmosphere, methanesulfonic acid (5 g, 52.17 mmol) was added at 0° C. and reacted for 0.5 h. After a product was generated as detected by LCMS, NaHCO₃ solid and water (10 mL) were added to adjust pH to 7-8. The mixed solution was extracted with DCM (50 mL) and water (30 mL), subjected to liquid separation, concentrated by rotary evaporation, mixed with silica gel and purified by column chromatography (DCM:MeOH=10:1) to give a green solid 8A (1.37 g, yield: 13%). MS-ESI: m/z 607.3 [M+H]^{+.}

Step 2 Compound 8A (1.3 g, 2.14 mmol) and iron powder (1.2 g, 21.4 mmol) were dissolved in EtOH (13 mL), a solution of NH₄Cl (1.14 g, 21.4 mmol) in H₂O (4.5 mL) was added, and the mixed solution was reacted at 60° C. for 2 h under nitrogen atmosphere. After the starting materials were substantially consumed as detected by LCMS, the reaction solution was filtered, extracted with water (10 mL) and EA (15 mL) and subjected to liquid separation. The organic phase was concentrated by rotary evaporation to give a yellow solid 8 (800 mg, yield: 65%).

MS-ESI: m/z 577.7 [M+H]^{+.}

¹H NMR (400 MHz, DMSO-d₆) δ 7.29 (d, J=10.1 Hz, 1H), 6.93-6.86 (m, 2H), 6.83 (t, J=2.0 Hz, 1H), 6.69 (t, J=2.5 Hz, 1H), 6.51-6.43 (m, 2H), 6.24 (dd, J=10.1, 1.9 Hz, 1H), 6.05 (s, 1H), 5.97 (dd, J=2.7, 1.7 Hz, 1H), 5.43-5.36 (m, 2H), 5.09-4.98 (m, 3H), 4.84-4.76 (m, 3H), 4.44 (dd, J=19.4, 6.0 Hz, 1H), 4.23-4.09 (m, 2H), 2.70-2.58 (m, 1H), 2.57-2.51 (m, 1H), 2.48-2.30 (m, 2H), 2.22-2.10 (m, 1H), 2.10-2.00 (m, 1H), 1.91-1.77 (m, 1H), 1.67-1.54 (m, 3H), 1.49 (s, 3H), 1.45-1.29 (m, 1H), 0.83 (s, 3H).

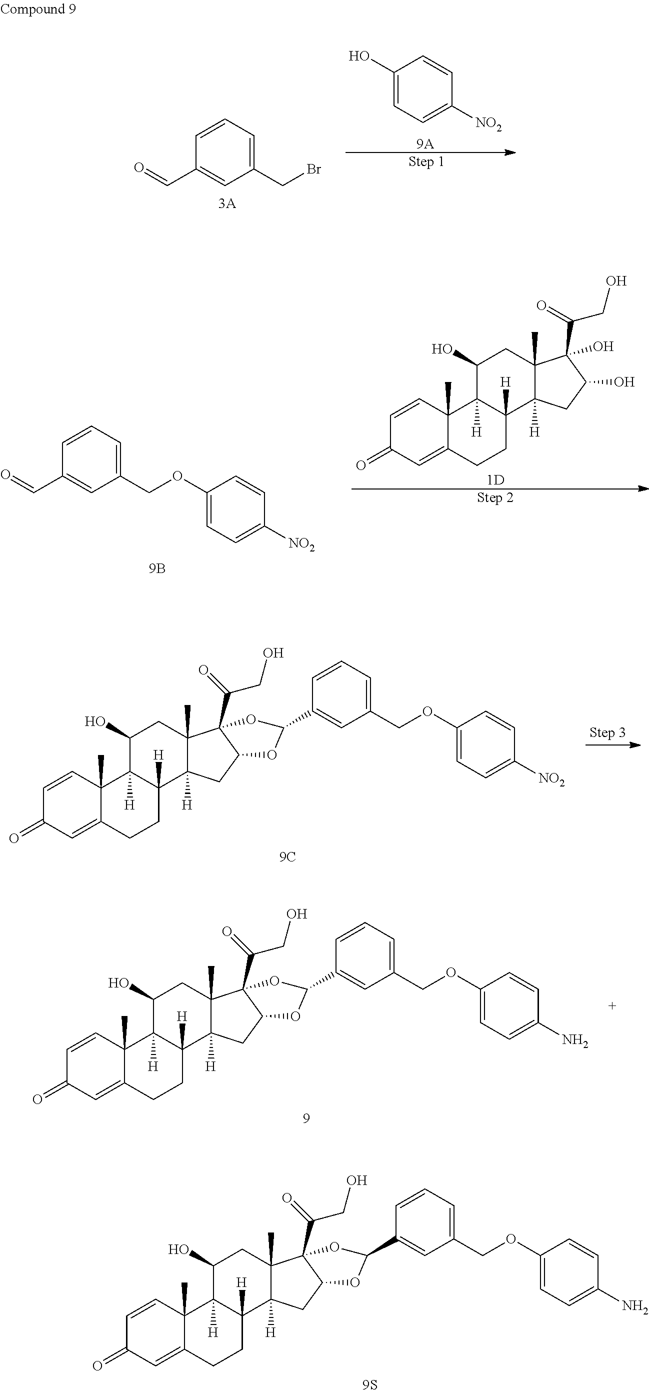

Step 1 Compound 9A (384 mg, 2.76 mmol) was added to a 250 mL three-necked flask, and DMF (5 mL) and K₂CO₃ (762 mg, 5.52 mmol) were added. Under nitrogen atmosphere, compound 3A (500 mg, 2.5 mmol) and a DMF solution (1 mL) were added dropwise in an ice-water bath. After addition, the mixed solution was heated to room temperature and reacted for 2 h. After the reaction was completed as detected by TLC (PE:EA=5:1), the reaction solution was poured into water (50 mL) and extracted with EA (100 mL). The organic phase was washed with water (50 mL×3), washed once with saturated NaCl, dried over anhydrous Na₂SO₄ and concentrated by rotary evaporation to give a white solid 9B (630 mg, yield: 97%). MS-ESI: m/z 280.2 [M+Na]^{+.}

Step 2 Compound 9B (100 mg, 0.39 mmol), compound 1D (146 mg, 0.39 mmol), acetonitrile (3 mL) and MgSO₄ (186 mg, 1.55 mmol) were added to a reaction flask. Under nitrogen atmosphere, the mixed solution was cooled to −20° C. Acetonitrile (0.5 mL) was dissolved in trifluoromethanesulfonic acid (175 mg, 1.16 mmol), cooled to room temperature and added dropwise to the reaction system. After addition, the mixed solution was stirred at −20° C. for 1.5 h. After the reaction was completed as detected by TLC (PE:EA=1:1), the reaction solution was poured into a saturated aqueous sodium bicarbonate solution (20 mL), and the product was extracted with EA (20 mL×2). The organic phase was washed with water (20 mL×3), dried over anhydrous sodium sulfate and concentrated by rotary evaporation to give a yellow foamy solid (290 mg, LCMS content: 80%, with negligible excess yield of crude product). MS-ESI: m/z 616.3 [M+H]^{+.}

Step 3 Compound 9C (140 mg, 0.227 mmol), iron powder (127 mg, 2.27 mmol) and NH₄Cl (121 mg, 2.27 mmol) were added to a reaction flask, and ethanol (1.5 mL) and water (0.4 mL) were added. Under nitrogen atmosphere, the mixed solution was heated to 60° C. and reacted for 1 h. After the reaction was completed as detected by LCMS, the reaction solution was cooled to room temperature and filtered, and water (20 mL) and EA (50 mL) were added to extract the product. The organic phase was washed with water (20 mL×2), dried over anhydrous sodium sulfate and concentrated to give an oil product (100 mg). Half of the oil product was purified by reversed-phase preparative chromatography and lyophilized to give compound 9 (11.7 mg, yield: 18%) and compound 9S (7.8 mg, yield: 12%).

### Compound 9

MS-ESI: m/z 586.3 [M+H]^{+.}

¹H NMR (400 MHz, DMSO-d₆) δ 8.87 (brs, 1H), 7.52 (s, 1H), 7.49-7.37 (m, 3H), 7.33 (d, J=10.0 Hz, 1H), 7.08 (d, J=8.0 Hz, 2H), 6.95 (d, J=8.6 Hz, 2H), 6.18 (dd, J=10.1, 1.9 Hz, 1H), 5.94 (s, 1H), 5.48 (s, 1H), 5.09 (s, 2H), 4.95 (d, J=4.5 Hz, 1H), 4.81 (d, J=3.4 Hz, 1H), 4.53 (d, J=19.5 Hz, 1H), 4.30 (s, 1H), 4.19 (d, J=19.4 Hz, 1H), 2.62-2.55 (m, 1H), 2.30 (s, 1H), 2.20-2.08 (m, 1H), 2.08-1.98 (m, 1H), 1.82-1.59 (m, 5H), 1.40 (s, 3H), 1.08-0.92 (m, 2H), 0.87 (s, 3H).

### Compound 9S

MS-ESI: m/z 586.3[M+H]^{+.}

¹H NMR (400 MHz, DMSO-d₆) δ 9.05 (brs, 1H), 7.47-7.35 (m, 3H), 7.32 (d, J=10.1 Hz, 1H), 7.27-7.20 (m, 1H), 7.16-7.07 (m, 2H), 7.07-6.94 (m, 2H), 6.17 (dd, J=10.1, 1.9 Hz, 1H), 6.13 (s, 1H), 5.95 (s, 1H), 5.31 (d, J=6.8 Hz, 1H), 5.08 (s, 2H), 4.97 (brs, 1H), 4.79 (d, J=3.4 Hz, 1H), 4.34-4.22 (m, 2H), 4.03 (d, J=19.1 Hz, 1H), 2.60-2.54 (m, 2H), 2.37-2.28 (m, 1H), 2.13-1.95 (m, 2H), 1.91-1.69 (m, 5H), 1.40 (s, 3H), 1.22-1.13 (m, 1H), 1.06 (dd, J=11.1, 3.3 Hz, 1H), 0.90 (s, 3H).

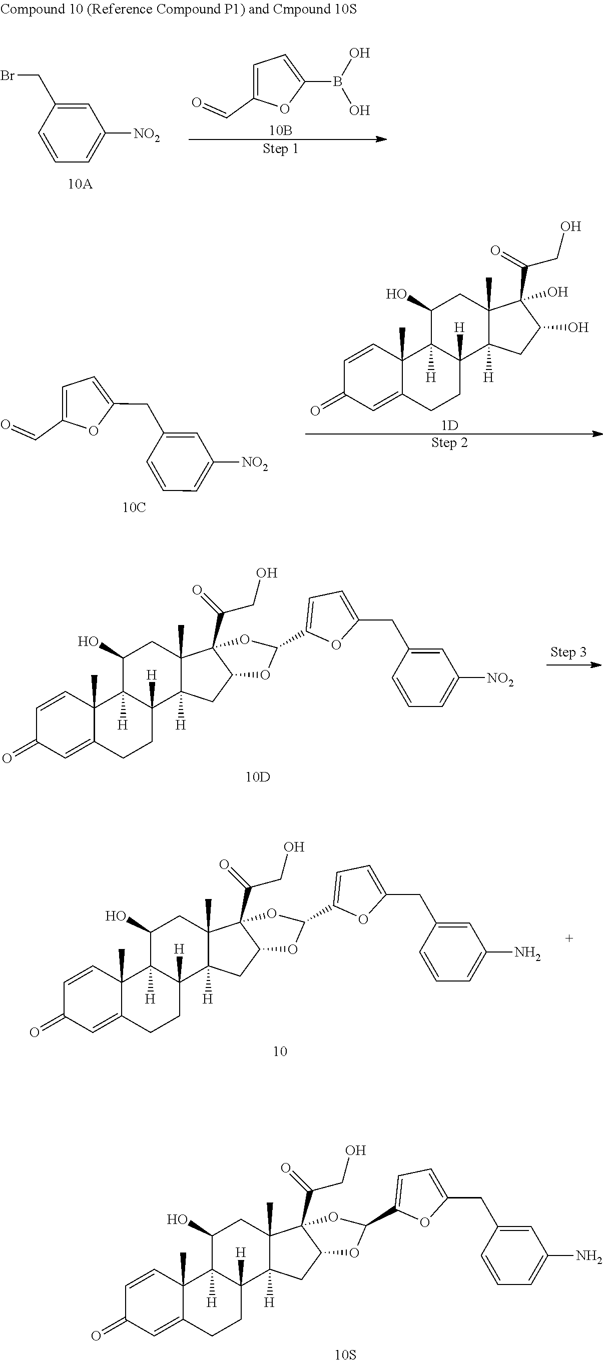

Step 1 Compound 10A (10 g, 46.3 mmol), compound 10B (6.5 g, 46.3 mmol) and K2CO₃ (19.2 g, 138.8 mmol) were added to THF (100 mL) and water (5 mL), and Pd(dppf)Cl₂ (3.4 g, 4.63 mmol) was added. Under nitrogen atmosphere, the external temperature was raised to 80° C., and the mixed solution was refluxed for 16 h. After the reaction was completed as detected by TLC (PE:EA=5:1), the reaction solution was cooled to room temperature and filtered, and water (300 mL) and EA (300 mL) were added to extract the product. The organic phase was washed with water (100 mL×3), dried over anhydrous sodium sulfate, concentrated by rotary evaporation, mixed with silica gel and purified by column chromatography (PE:EA=10:1) to give a white foamy solid 10C (3.1 g, yield: 29%). MS-ESI: m/z 232.1 [M+H]^{+.}

Step 2 Compound 10C (100 mg, 0.43 mmol), compound 1D (163 mg, 0.43 mmol), acetonitrile (3 mL) and MgSO₄ (208 mg, 1.73 mmol) were added to a reaction flask. Under nitrogen atmosphere, the mixed solution was cooled to 0° C. Acetonitrile (0.5 mL) was dissolved in methanesulfonic acid (125 mg, 1.3 mmol) and added dropwise to the reaction system. After addition, the mixed solution was stirred at 0° C. to 10° C. for 1 h. After half of the product was detected by LCMS, the reaction solution was poured into a saturated aqueous sodium bicarbonate solution (20 mL), and the product was extracted with EA (20 mL×2). The organic phase was washed with water (20×3), dried over anhydrous sodium sulfate, concentrated by rotary evaporation and separated by preparative plate chromatography (PE:EA=1:1) to give a white solid 10D (100 mg, yield: 39%). MS-ESI: m/z 590.3 [M+H]^{+.}

Step 3 Compound 10D (100 mg, 0.17 mmol), iron powder (95 mg, 1.7 mmol) and NH₄Cl (91 mg, 1.7 mmol) were added to a reaction flask, and ethanol (1 mL) and water (0.25 mL) were added. Under nitrogen atmosphere, the mixed solution was heated to 60° C. and reacted for 1 h. After the reaction was completed as detected by LCMS, the reaction solution was cooled to room temperature and filtered, and water (20 mL) and EA (50 mL) were added to extract the product. The organic phase was washed with water (20 mL×2), dried over anhydrous sodium sulfate, concentrated by rotary evaporation and purified by preparative thin layer chromatography (PE:EA=1:2) to give a white solid (40 mg). The solid was purified by reversed-phase preparative chromatography, and the pure product was lyophilized to give compound 10 (5 mg, yield: 5%) and compound 10S (8.8 mg, yield: 9%).

### Compound 10

MS-ESI: m/z 560.2 [M+H]^{+.}

¹H NMR (400 MHz, DMSO-d₆) δ 7.34 (d, J=10.1 Hz, 1H), 7.02-6.94 (m, 1H), 6.66 (brs, 3H), 6.50 (d, J=3.2 Hz, 1H), 6.21 (dd, J=10.1, 1.9 Hz, 1H), 6.12 (d, J=3.2 Hz, 1H), 5.97 (s, 1H), 5.54 (s, 1H), 4.89 (d, J=5.3 Hz, 1H), 4.78 (brs, 1H), 4.44 (d, J=19.4 Hz, 1H), 4.28 (s, 1H), 4.14 (d, J=19.5 Hz, 1H), 3.83 (s, 2H), 2.61-2.53 (m, 1H), 2.36-2.29 (m, 1H), 2.16-1.95 (m, 3H), 1.81-1.54 (m, 5H), 1.40 (s, 3H), 1.03-0.91 (m, 2H), 0.84 (s, 3H).

### Compound 10S

MS-ESI: m/z 560.2 [M+H]^{+.}

¹H NMR (400 MHz, DMSO-d₆) δ 7.30 (d, J=10.1 Hz, 1H), 7.18-7.10 (m, 1H), 6.80-6.62 (m, 3H), 6.33 (d, J=3.3 Hz, 1H), 6.16 (dd, J=10.1, 1.9 Hz, 1H), 6.09 (s, 1H), 6.06 (d, J=3.2 Hz, 1H), 5.93 (s, 1H), 5.16 (d, J=6.1 Hz, 1H), 4.78 (s, 1H), 4.28 (s, 1H), 4.15 (d, J=19.3 Hz, 1H), 4.00 (d, J=19.3 Hz, 1H), 3.83 (s, 2H), 2.58-2.53 (m, 1H), 2.32-2.26 (m, 1H), 2.11-1.94 (m, 3H), 1.85-1.78 (m, 1H), 1.78-1.55 (m, 4H), 1.38 (s, 3H), 1.16-1.08 (m, 1H), 1.00 (dd, J=11.0, 3.5 Hz, 1H), 0.87 (s, 3H).

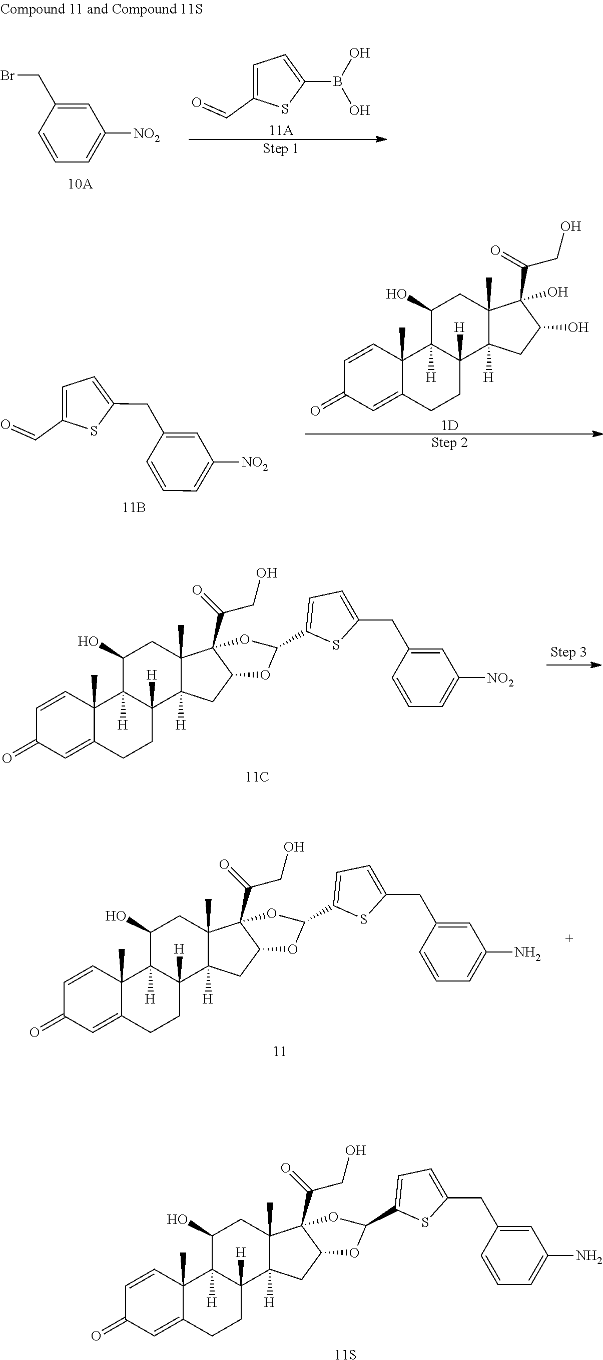

Step 1 Compound 10A (8 g, 37 mmol), compound 11A (5.8 g, 37 mmol) and K₂CO₃ (15.3 g, 111 mmol) were added to THF (80 mL), and water (4 mL) and Pd(dppf)Cl₂ (2.7 g, 3.7 mmol) were added. Under nitrogen atmosphere, the external temperature was raised to 80° C., and the mixed solution was refluxed for 16 h. After the reaction was completed as detected by TLC (PE:EA=5:1), the reaction solution was cooled to room temperature and filtered, and water (300 mL) and EA (300 mL) were added to extract the product. The organic phase was washed with water (100 mL×3), dried over anhydrous sodium sulfate, concentrated by rotary evaporation, mixed with silica gel and purified by column chromatography (PE:EA=10:1) to give a white foamy solid 11B (2.86 g, yield: 31%). MS-ESI: m/z 248.1 [M+H]^{+.}

Step 2 Compound 11B (200 mg, 0.81 mmol), compound 1D (304 mg, 0.81 mmol), acetonitrile (6 mL) and MgSO₄ (388 mg, 3.24 mmol) were added to a reaction flask. Under nitrogen atmosphere, the mixed solution was cooled to 0° C. Acetonitrile (1 mL) was dissolved in methanesulfonic acid (233 mg, 2.43 mmol) and added dropwise to the reaction system. After addition, the mixed solution was stirred at 0° C. to 10° C. for 6 h. After the content of the product was 70% as detected by LCMS, the reaction solution was poured into a saturated aqueous sodium bicarbonate solution (40 mL), and the product was extracted with EA (40 mL×2). The organic phase was washed with water (40 mL×3), dried over anhydrous sodium sulfate and concentrated by rotary evaporation to give a yellow solid 11C (480 mg, with negligible yield of crude product). MS-ESI: m/z 606.2 [M+H]^{+.}

Step 3 Compound 11C (240 mg, 0.40 mmol), iron powder (222 mg, 4.00 mmol) and NH₄Cl (212 mg, 4.00 mmol) were added to a reaction flask, and ethanol (3 mL) and water (0.75 mL) were added. Under nitrogen atmosphere, the mixed solution was heated to 60° C. and reacted for 1 h. After the reaction was completed as detected by LCMS, the reaction solution was cooled to room temperature and filtered, and water (40 mL) and EA (100 mL) were added to extract the product. The organic phase was washed with water (40 mL×2), dried over anhydrous sodium sulfate, concentrated by rotary evaporation and purified by preparative thin layer chromatography (PE:EA=1:2) to give a white solid (40 mg). The solid was purified by reversed-phase preparative chromatography and lyophilized to give compound 11 (15 mg, yield: 6%) and compound 11S (4.4 mg, yield: 2%).

### Compound 11

MS-ESI: m/z 576.2 [M+H]^{+.}

¹H NMR (400 MHz, DMSO-d₆) δ 7.31 (d, J=10.1 Hz, 1H), 7.12-7.06 (m, 1H), 7.02 (t, J=8.0 Hz, 1H), 6.73 (d, J=3.5 Hz, 1H), 6.69-6.53 (m, 3H), 6.18 (dd, J=10.1, 1.9 Hz, 1H), 5.97 (s, 1H), 5.73 (s, 1H), 4.88 (d, J=4.3 Hz, 1H), 4.80 (s, 1H), 4.48 (d, J=19.4 Hz, 1H), 4.29 (d, J=4.2 Hz, 1H), 4.15 (d, J=19.5 Hz, 1H), 3.98 (s, 2H), 2.60-2.54 (m, 1H), 2.37-2.28 (m, 1H), 2.18-1.94 (m, 2H), 1.79-1.54 (m, 5H), 1.39 (s, 3H), 1.02-0.91 (m, 1H), 0.88 (dd, J=11.2, 3.5 Hz, 1H), 0.84 (s, 3H).

### Compound 11S

MS-ESI: m/z 576.3 [M+H]^{+.}

¹H NMR (400 MHz, DMSO-d₆) δ 7.31 (d, J=10.1 Hz, 1H), 7.15-7.08 (m, 1H), 7.00 (d, J=3.5 Hz, 1H), 6.75 (d, J=3.5 Hz, 1H), 6.74-6.59 (m, 3H), 6.29 (s, 1H), 6.17 (dd, J=10.1, 1.9 Hz, 1H), 5.94 (s, 1H), 5.20 (d, J=7.0 Hz, 1H), 4.78 (s, 1H), 4.36 (d, J=19.2 Hz, 1H), 4.29 (s, 1H), 4.10-3.94 (m, 3H), 2.59-2.53 (m, 1H), 2.32-2.26 (m, 1H), 2.11-1.94 (m, 3H), 1.86-1.61 (m, 5H), 1.38 (s, 3H), 1.21-1.12 (m, 1H), 1.03 (dd, J=11.2, 3.4 Hz, 1H), 0.86 (s, 3H).

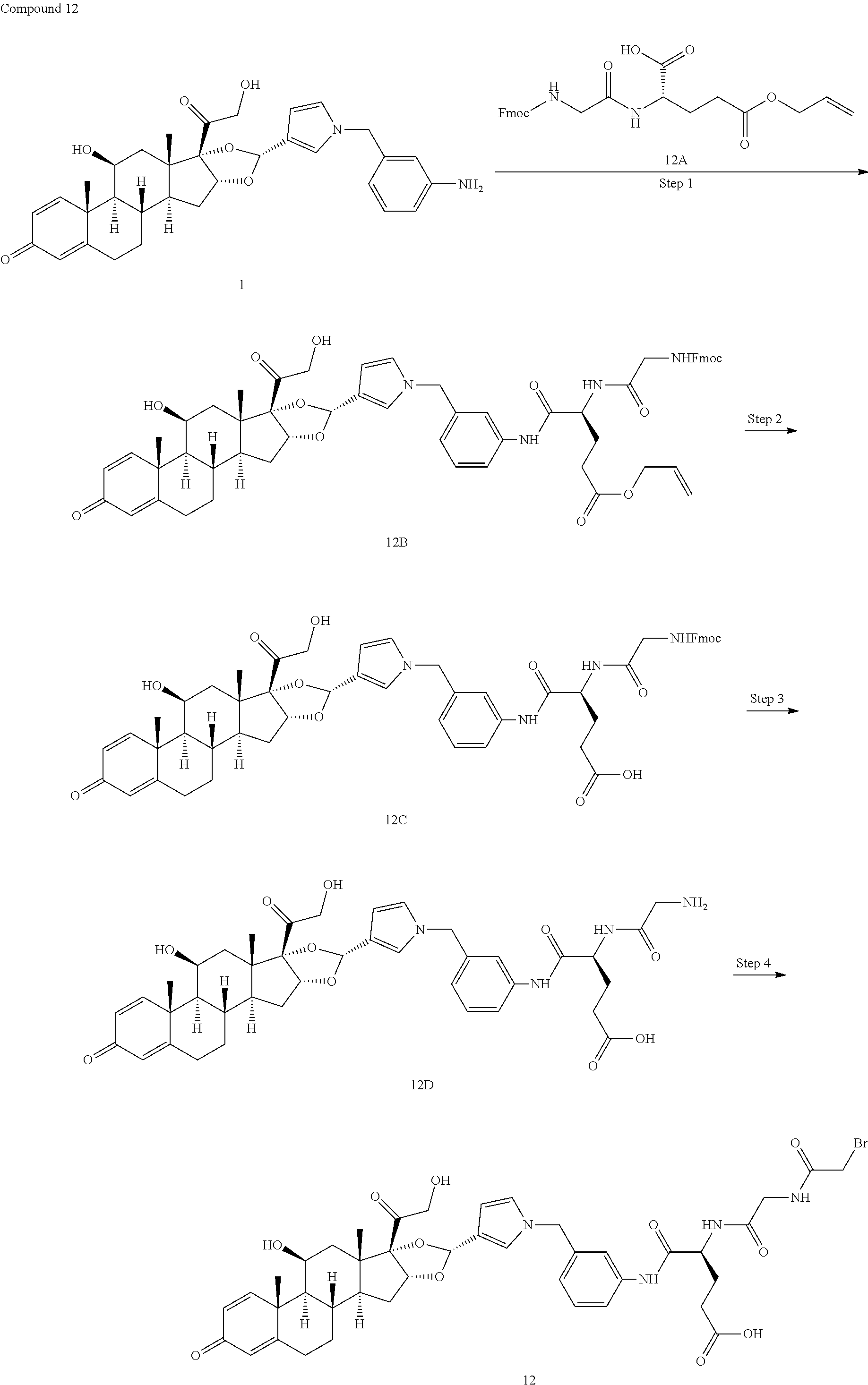

Step 1 Compound 1 (500 mg, 0.896 mmol, 1.0 eq) was added to DMF (5 mL), and then compound 12A (417 mg, 0.896 mmol, 1.0 eq), 2,6-lutidine (288 mg, 2.69 mmol, 3.0 eq) and HATU (410 mg, 1.08 mmol, 1.2 eq) were added. The mixed solution was reacted at room temperature for 2 h. After the reaction was completed as detected by LCMS, the reaction solution was added dropwise to water (50 mL), and a solid was precipitated. The resulting mixture was filtered, and the filter cake was washed with water (20 mL) and lyophilized to remove water to give a light yellow solid 12B (750 mg, yield: 83%). MS-ESI: m/z 1007.3 [M+H]^{+.}

Step 2 Compound 12B (750 mg, 0.745 mmol, 1.0 eq) was added to THF (8 mL), and then Pd(PPh₃)₄ (172 mg, 0.149 mmol, 0.2 eq) and N-methylmorpholine (376 mg, 3.73 mmol, 5 eq) were added. The mixed solution was reacted at room temperature for 2 h. After the reaction was completed as detected by LCMS, the reaction solution was directly purified by reversed-phase column chromatography (MeCN/H₂O=5% to 60%) to give a yellow powdery solid 12C (650 mg, yield: 90%). MS-ESI: m/z 967.6 [M+H]^{+.}

Step 3 Compound 12C (350 mg, 0.362 mmol, 1.0 eq) was added to DCM (4.5 mL) and cooled to 0° C., and then diethylamine (1.5 mL) was added. The mixed solution was heated to room temperature and reacted for 2 h. After the reaction was completed as detected by LCMS, the reaction solution was added to methyl tert-butyl ether (150 mL), and a solid was precipitated. After the resulting mixture was left to stand, the supernatant was poured out. The remaining solid was dissolved in DMF and purified by reversed-phase column chromatography (MeCN/H₂O=5% to 40%) to give a yellow powdery solid 12D (60 mg, yield: 22%).

Step 4 Compound 12D (60 mg, 0.08 mmol, 1.0 eq) was added to THF/H₂O (4 mL/1 mL) and cooled to 0° C., and then triethylamine (162 mg, 1.6 mmol, 20.0 eq) and bromoacetyl bromide (129 mg, 0.64 mmol, 8.0 eq, diluted in THF 0.1 mL) were added. The mixed solution was reacted at 0° C. for 5 min. After the reaction was completed as detected by LCMS, the reaction solution was directly purified by preparative chromatography to give a white powdery solid 12 (13 mg, yield: 19%).

MS-ESI: m/z 865.3 [M+H]^{+.}

¹H NMR (400 MHz, DMSO-d₆) δ 9.98 (s, 1H), 8.56-8.49 (m, 1H), 8.25 (d, J=7.7 Hz, 1H), 7.55-7.50 (m, 1H), 7.50-7.44 (m, 1H), 7.34-7.28 (m, 1H), 7.23 (t, J=7.8 Hz, 1H), 6.98-6.84 (m, 2H), 6.76-6.70 (m, 1H), 6.17 (dd, J=10.1, 1.9 Hz, 1H), 6.08 (dd, J=2.8, 1.7 Hz, 1H), 5.94 (s, 1H), 5.36 (s, 1H), 5.01 (s, 2H), 4.84-4.72 (m, 2H), 4.48-4.35 (m, 3H), 4.32-4.26 (m, 1H), 4.14 (d, J=19.4 Hz, 1H), 3.93 (s, 2H), 3.79 (dd, J=5.6, 2.3 Hz, 2H), 2.60-2.53 (m, 1H), 2.32-2.21 (m, 2H), 1.89-1.53 (m, 6H), 1.38 (s, 3H), 1.08-0.93 (m, 2H), 0.83 (s, 3H).

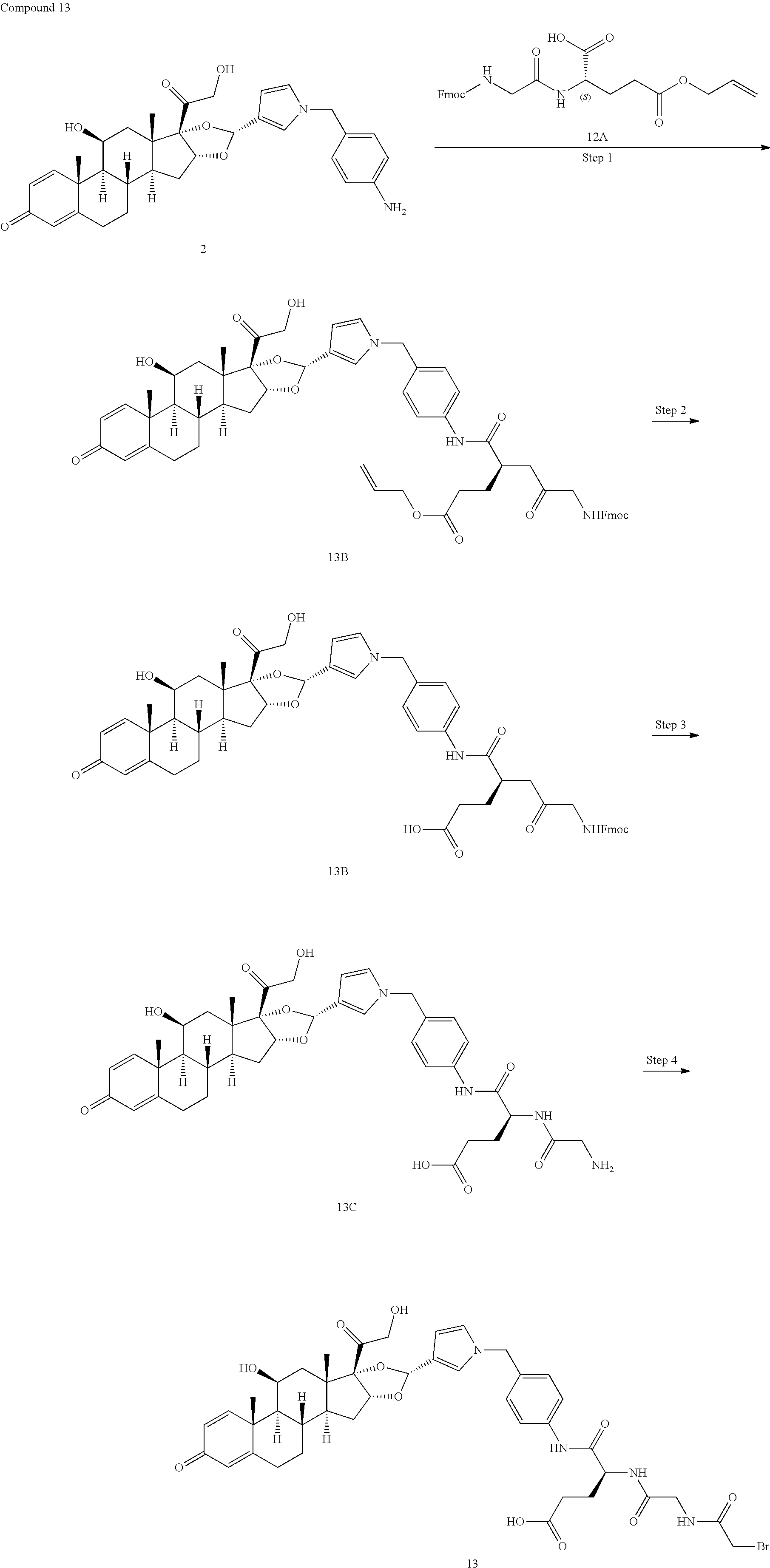

Step 1 Compound 2 (1.5 g, 2.68 mmol), compound 12A (963 mg, 2.06 mmol) and 2,6-lutidine (663 mg, 6.19 mmol) were added to a three-necked flask (100 mL), DMF (20 mL) was added for dissolution, and HATU (942 mg, 2.48 mmol) was added. The mixed solution was stirred for 17 h. After the reaction was completed as detected by LCMS, the reaction solution was slowly added to water (200 mL), and a large amount of yellow solid was precipitated. The resulting mixture was filtered, and the filter cake was washed with water (100 mL) and lyophilized to give a yellow solid 13A (1.1 g, yield: 53%). MS-ESI: m/z 1007.4 [M+H]^{+.}

Step 2 Compound 13A (1 g, 0.99 mmol), N-methylmorpholine (999 mg, 9.9 mmol) and Pd(PPh₃)₄ (229 mg, 0.20 mmol) were added to a 25 mL three-necked flask, and THF (10 mL) was added for dissolution. The mixed solution was purged with nitrogen, cooled to 0° C. and reacted for 2 h. After the reaction was completed as detected by LCMS, the reaction solution was directly purified by reversed-phase column chromatography (H₂O/CH₃CN=57/43) to give a product, which was lyophilized to give a white powder 13B (350 mg, yield: 36%). MS-ESI: m/z 967.4 [M+H]^{+.}

Step 3 Compound 13B (350 mg, 0.36 mmol) was dissolved in DCM (6 mL), diethylamine (2 mL) was added, and the mixed solution was stirred for 2 h. After the reaction was completed as detected by LCMS, the reaction solution was added dropwise to PE (60 mL), and a large amount of yellow solid was precipitated. After the resulting mixture was left to stand for 5 min, the supernatant was poured out. The solid was completely dissolved in DMF and then purified by reversed-phase column chromatography (water/acetonitrile=75/25) to give a product, which was lyophilized to give a white powder 13C (80 mg, yield: 30%). MS-ESI: m/z 745.4 [M+H]^{+.}

Step 4 Compound 13C (80 mg, 0.107 mmol, 1.0 eq) was dissolved in a system of THF (3 mL) and H₂O (0.6 mL). The resulting solution was purged with nitrogen and cooled to −5° C. in an ice-ethanol bath. Then triethylamine (217 mg, 2.15 mmol, 2.0 eq) and bromoacetyl bromide (172 mg, 0.86 mmol, 8.0 eq) were added. The mixed solution was stirred for 5 min. After the reaction was completed as detected by LCMS, the reaction solution was purified by preparative chromatography at a low temperature and lyophilized to give a white powder 13 (10 mg, yield: 11%).

MS-ESI: m/z 865.2 [M+H]^{+.}

¹H NMR (400 MHz, DMSO-d₆) δ 9.97 (s, 1H), 8.52 (t, J=5.6 Hz, 1H), 8.26 (d, J=7.9 Hz, 1H), 7.55 (d, J=8.6 Hz, 2H), 7.31 (d, J=10.1 Hz, 1H), 7.15 (d, J=8.6 Hz, 2H), 6.92 (t, J=2.0 Hz, 1H), 6.72 (t, J=2.5 Hz, 1H), 6.17 (dd, J=10.1, 1.9 Hz, 1H), 6.07 (dd, J=2.8, 1.7 Hz, 1H), 5.94 (s, 1H), 5.35 (s, 1H), 4.98 (s, 2H), 4.84-4.70 (m, 2H), 4.48-4.37 (m, 2H), 4.29 (s, 1H), 4.14 (d, J=19.4 Hz, 1H), 3.94 (s, 2H), 3.80 (dd, J=5.6, 1.7 Hz, 2H), 2.59-2.52 (m, 2H), 2.36-2.21 (m, 3H), 2.16-1.91 (m, 3H), 1.89-1.62 (m, 5H), 1.58 (dd, J=14.6, 5.2 Hz, 1H), 1.39 (s, 3H), 1.10-0.93 (m, 2H), 0.83 (s, 3H).

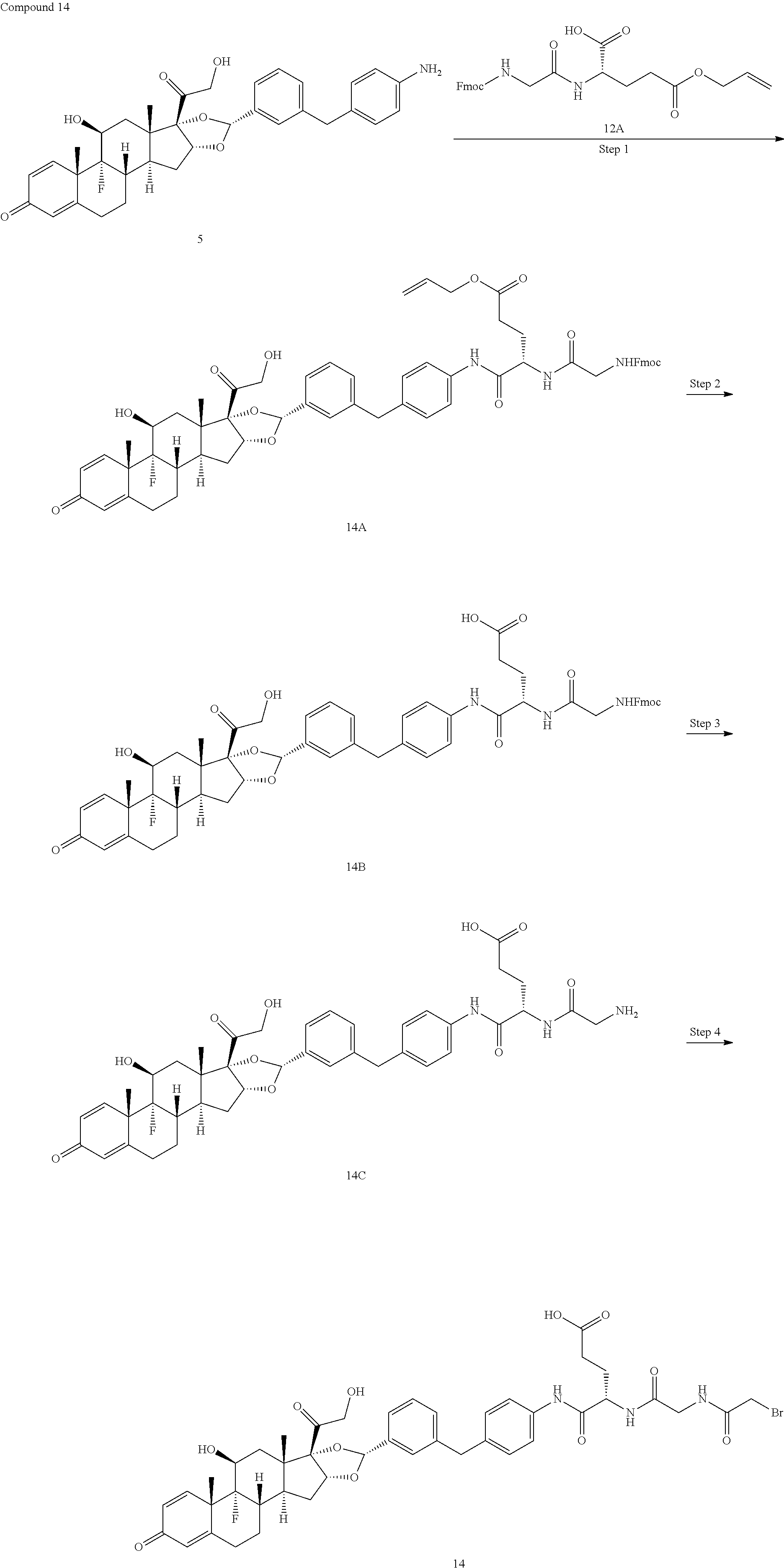

Step 1 Under nitrogen atmosphere, to compound 5 (200 mg, 0.34 mmol), compound 12A (159 mg, 0.34 mmol), DMF (6 mL) and DIEA (110 mg, 0.85 mmol) was added dropwise a solution of HATU (155 mg, 0.41 mmol) in DMF (0.5 mL) at 0° C. After addition, the mixed solution was reacted at 0° C. for 0.5 h. After the reaction was completed as detected by TLC (PE/EA=1/5), the reaction solution was added to water (60 mL), a solid was precipitated, and the resulting mixture was extracted with ethyl acetate (70 mL) and subjected to liquid separation. The residue was purified by silica gel column chromatography (PE:EA=3:7) to give a white solid 14A (270 mg, yield: 76%).

Step 2 Under nitrogen atmosphere, to a solution of compound 14A (200 mg, 0.19 mmol) in THF (4 mL) were added N-methylmorpholine (195 mg, 1.93 mmol) and Pd(PPh₃)₄ (44.6 mg, 0.038 mmol) at 0° C. The mixed solution was reacted at room temperature for 1 h. After the reaction was completed as detected by LCMS, the reaction solution was directly purified by reversed-phase column chromatography (CH₃CN:H₂O=39:61) to give a white powder 14B (145 mg, yield: 75%). MS-ESI: m/z 996.4 [M+H]^{+.}

Step 3 Under nitrogen atmosphere, to a solution of compound 14B (160 mg, 0.16 mmol) in dichloromethane (4 mL) was added diethylamine (1 mL). The mixed solution was reacted at 0° C. for 1 h. After the starting materials were completely consumed as detected by LCMS, the reaction solution was extracted with PE (40 mL) and water (30 mL). The aqueous phase was lyophilized, and the solid was directly purified by preparative chromatography to give a white solid 14C (55 mg, yield: 44%). MS-ESI: m/z 774.4 [M+H]^{+.}

Step 4 Under nitrogen atmosphere, to a solution of compound 14C (40 mg, 0.052 mmol) and triethylamine (52.3 mg, 0.52 mmol) in THF (4 mL) and H₂O (0.5 mL) was added bromoacetyl bromide (41.7 mg, 0.207 mmol). The mixed solution was reacted at 0° C. for 10 min. After the starting materials were completely consumed as detected by LCMS, the reaction solution was directly purified by preparative chromatography to give a white solid 14 (21 mg, yield: 45%).

MS-ESI: m/z 894.2 [M+H]^{+.}

¹H NMR (400 MHz, DMSO-d₆) δ 9.88 (s, 1H), 8.53 (t, J=5.6 Hz, 1H), 8.26 (d, J=7.8 Hz, 1H), 7.48 (d, J=8.2 Hz, 2H), 7.34-7.21 (m, 5H), 7.12 (d, J=8.2 Hz, 2H), 6.25 (dd, J=10.0, 1.9 Hz, 1H), 6.10 (s, 1H), 5.45 (d, J=6.7 Hz, 2H), 4.93-4.85 (m, 1H), 4.50 (d, J=19.5 Hz, 1H), 4.45-4.37 (m, 1H), 4.28-4.14 (m, 2H), 3.93 (s, 2H), 3.85-3.76 (m, 5H), 2.71-2.60 (m, 1H), 2.59-2.53 (m, 1H), 2.44-2.36 (m, 1H), 2.35-2.13 (m, 3H), 2.08 (d, J=13.5 Hz, 1H), 2.04-1.92 (m, 1H), 1.89-1.76 (m, 2H), 1.74-1.57 (m, 3H), 1.51 (s, 3H), 1.44-1.31 (m, 1H), 0.87 (s, 3H).

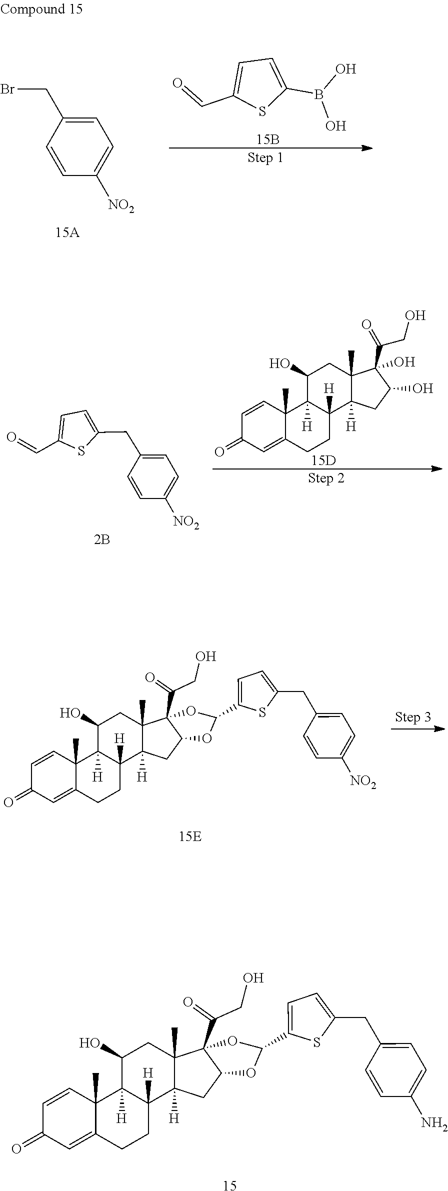

Step 1 Compound 15A (5 g, 23.1 mmol), 15B (3.6 g, 23.1 mmol) and K₂CO₃ (9.6 g, 69.3 mmol) were dissolved in THF (50 mL), and water (5 mL) was added. Under nitrogen atmosphere, Pd(dppf)Cl₂ (3.38 g, 4.6 mmol) was added, the external temperature was raised to 80° C., and the mixed solution was refluxed for 16 h. After the reaction was completed as detected by TLC (PE:EA=5:1), the reaction solution was cooled to room temperature and filtered, and water (300 mL) and EA (300 mL) were added to extract the product. The organic phase was washed with water (100 mL×3), dried over anhydrous sodium sulfate, mixed with silica gel, concentrated and purified by column chromatography (PE:EA=15:1) to give a yellow solid 15C (830 mg, yield: 14%). MS-ESI: m/z 248.1 [M+H]^{+.}

Step 2 Under nitrogen atmosphere, compound 15C (800 mg, 3.2 mmol), 15D (1.2 g, 3.2 mmol), acetonitrile (20 mL) and MgSO₄ (15.5 g, 12.9 mmol) were added to a reaction flask. The mixed solution was cooled to −20° C., and a solution of trifluoromethanesulfonic acid (1.46 g, 9.7 mmol) in acetonitrile (5 mL) was cooled and added dropwise to the reaction system. After addition, the resulting solution was stirred at −20° C. for 1 h. After the reaction was completed as detected by LCMS, the reaction solution was poured into a saturated aqueous sodium bicarbonate solution (100 mL), and the product was extracted with EA (100 mL×2). The organic phase was washed with water (50 mL×3), dried over anhydrous sodium sulfate, concentrated, mixed with silica gel and purified by column chromatography to give a yellow solid 15E (1 g, yield: 51%). MS-ESI: m/z 606.3 [M+H]^{+.}

Step 3 Compound 15E (100 mg, 0.165 mmol), iron powder (93 mg, 1.65 mmol) and NH₄Cl (88 mg, 1.65 mmol) were added to a reaction flask, and ethanol (0.8 mL) and water (0.2 mL) were added. Under nitrogen atmosphere, the mixed solution was heated to 60° C. and reacted for 1 h. After the reaction was completed as detected by LCMS, the reaction solution was cooled to room temperature and filtered, and water (40 mL) and EA (100 mL) were added to extract the product. The organic phase was washed with water (40 mL×2), purified by preparative thin layer chromatography (PE:EA=1:3) and lyophilized to give a yellow solid 15 (46 mg, yield: 48%).

MS-ESI: m/z 576.3 [M+H]^{+.}

¹H NMR (400 MHz, DMSO-d₆) δ 7.36-7.28 (m, 1H), 7.04 (d, J=3.5 Hz, 1H), 6.84 (d, J=8.3 Hz, 2H), 6.66 (d, J=3.5 Hz, 1H), 6.45 (d, J=8.4 Hz, 2H), 6.19 (dd, J=10.1, 1.9 Hz, 1H), 6.00 (s, 1H), 5.73 (s, 1H), 5.07 (t, J=5.9 Hz, 1H), 4.93-4.85 (m, 3H), 4.82-4.76 (m, 1H), 4.48 (dd, J=19.5, 6.3 Hz, 1H), 4.34-4.26 (m, 1H), 4.15 (dd, J=19.5, 5.6 Hz, 1H), 3.88 (s, 2H), 2.60-2.52 (m, 1H), 2.38-2.30 (m, 1H), 2.17-1.96 (m, 2H), 1.78-1.57 (m, 5H), 1.39 (s, 3H), 1.06-0.93 (m, 1H), 0.91-0.86 (m, 1H), 0.84 (s, 3H).

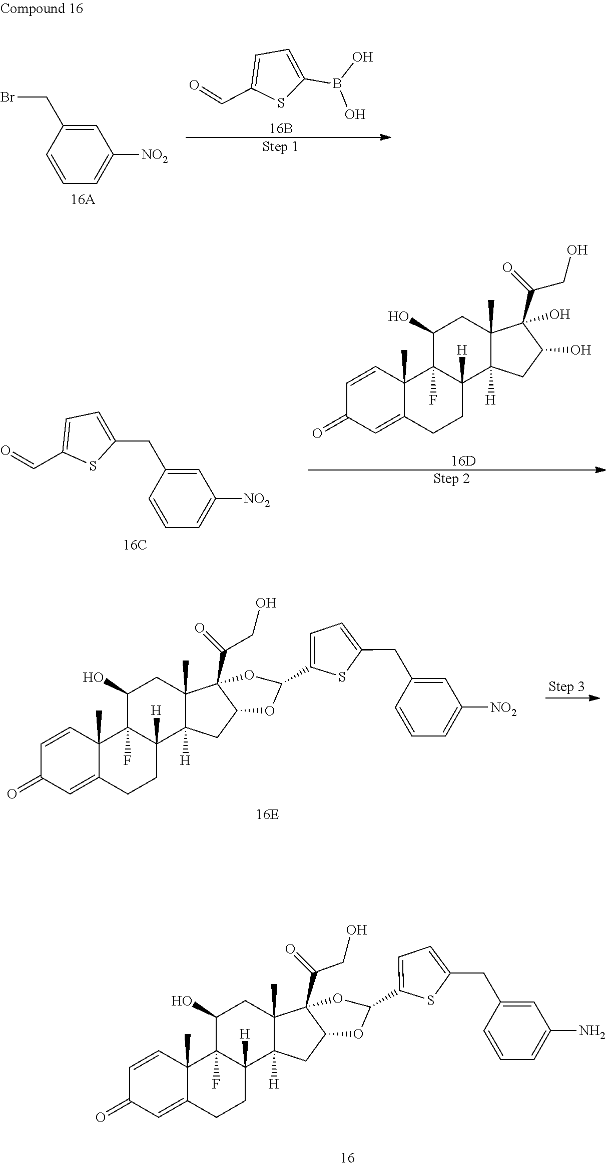

Step 1 Compound 16A (7.6 g, 35.2 mmol) and 16B (5.5 g, 35.2 mmol) were added to a 500 mL single-neck flask and dissolved in THF (60 mL), and K₂CO₃ (14.6 g, 105.6 mmol) and H₂O (10 mL) were added. Under nitrogen atmosphere, Pd(dppf)Cl₂ (5.2 g, 7.04 mmol) was added. The resulting solution was refluxed at 80° C. for 2 h. After the starting materials were substantially consumed as detected by LCMS, the reaction solution was filtered, and the mother liquor was extracted with EA (100 mL) and H₂O (70 mL) and subjected to liquid separation. The organic phase was dried, concentrated by rotary evaporation and purified by column chromatography (PE:EA=3:1) to give a yellow solid product 16C (1.2 g, yield: 14%). MS-ESI: m/z 248.1 [M+H]^{+.}

Step 2 Compound 16C (1.12 g, 4.53 mmol) and 16D (1.79 g, 4.53 mmol) were dissolved in MeCN (10 mL), and MgSO₄ (1.64 g, 13.6 mmol) was added. Under nitrogen atmosphere, TfOH (1.36 g, 9.07 mmol) was added at 0° C., and the mixed solution was reacted for 3 h. After the product was generated as detected by LCMS, the reaction solution was filtered, directly concentrated by rotary evaporation, mixed with silica gel and purified (DCM:MeOH=13:1) to give an orange-red solid 16E (750 mg, yield: 26%). MS-ESI: m/z 624.3 [M+H]^{+.}

Step 3 Compound 16E (700 mg, 1.124 mmol) and Fe (629 mg, 11.24 mmol) were dissolved in EtOH (9 mL), a solution of NH₄Cl (596 mg, 11.24 mmol) in H₂O (3 mL) was added, and the mixed solution was reacted at 60° C. for 2 h under nitrogen atmosphere. After the starting materials were substantially consumed as detected by LCMS, the reaction solution was filtered, extracted with water (10 mL) and EA (15 mL) and subjected to liquid separation. The organic phase was concentrated by rotary evaporation to give a yellow solid 16 (600 mg, yield: 90%).

MS-ESI: m/z 594.3 [M+H]^{+.}

¹H NMR (400 MHz, DMSO-d₆) δ 7.29 (d, J=10.1 Hz, 1H), 7.18 (t, J=7.8 Hz, 1H), 7.08 (d, J=3.5 Hz, 1H), 6.89 (d, J=7.6 Hz, 1H), 6.86-6.80 (m, 2H), 6.75 (d, J=3.5 Hz, 1H), 6.24 (dd, J=10.1, 1.9 Hz, 1H), 6.05 (s, 1H), 5.74 (s, 1H), 5.43 (s, 1H), 4.92-4.85 (m, 1H), 4.49 (d, J=19.4 Hz, 1H), 4.22-4.13 (m, 2H), 4.06 (s, 2H), 2.71-2.52 (m, 2H), 2.49-2.40 (m, 1H), 2.40-2.31 (m, 1H), 2.20-2.07 (m, 1H), 2.05-1.95 (m, 1H), 1.88-1.79 (m, 1H), 1.71-1.57 (m, 3H), 1.50 (s, 3H), 1.44-1.29 (m, 1H), 0.85 (s, 3H).

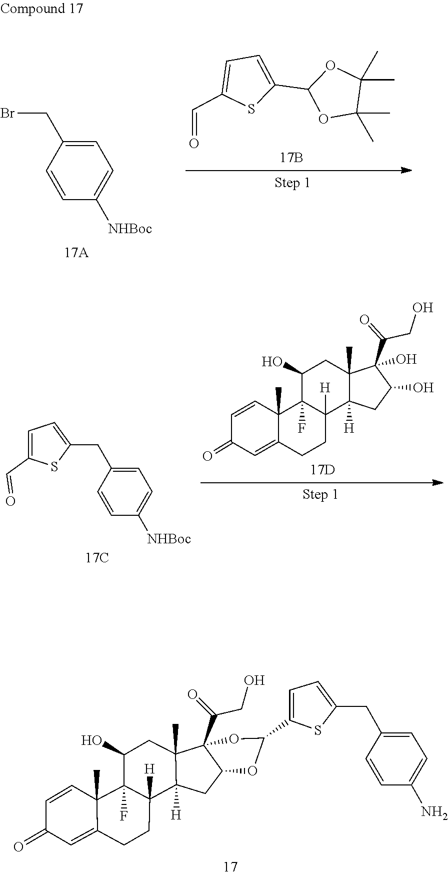

Step 1 Compound 17A (654 mg, 2.29 mmol), compound 17B (500 mg, 2.08 mmol) and Pd(PPh₃)₄ (120 mg, 104 μmol) were dissolved in DME (13.3 mL). The mixed solution was purged three times with nitrogen and stirred for 15 min. Sodium carbonate (1.52 g, 14.3 mmol) was dissolved in water (4.80 mL) and added to the reaction solution. The resulting solution was purged three times with nitrogen, heated to 90° C. and stirred for 16 h. After the starting materials were completely consumed as detected by LCMS, the mixture was cooled to room temperature and poured into water (15.0 mL). The resulting mixture was extracted with ethyl acetate (15.0 mL, 10.0 mL). The organic phases were combined, washed with a saturated aqueous sodium chloride solution (10.0 mL), dried over anhydrous sodium sulfate, filtered and concentrated to give a crude product, which was purified by reversed-phase preparative chromatography [water (NH₃H₂O+NH₄HCO3)-acetonitrile] to give a yellow solid, compound 17C (300 mg, yield: 45.4%). MS-ESI: m/z 262.1 [M+H-C4H₈]^{+.}

Step 2 Compound 17C (100 mg, 315 μmol) and compound 17D (124 mg, 315 μmol) were dissolved in acetonitrile (0.600 mL), and perchloric acid (361 mg, 2.52 mmol, 217 μL, purity: 70.0%) was added dropwise at 0° C. The mixed solution was gradually heated to 25° C. and stirred for 12 h. After there were 26% of starting materials left and 50% of the product as detected by LCMS, the reaction solution was quenched with a saturated aqueous sodium bicarbonate solution (6.00 mL) and extracted with ethyl acetate (6.00 mL, 3.00 mL, 3.00 mL). The organic phases were combined, washed with a saturated aqueous sodium chloride solution (5.00 mL), dried over anhydrous sodium sulfate, filtered and concentrated to give a crude product, which was purified by reversed-phase preparative chromatography [water (NH₄HCO3)-acetonitrile] to give a white solid, compound 17 (9.96 mg, yield: 5.30%). MS-ESI: m/z 594.1 [M+H]^{+.}

¹H NMR (400 MHz, DMSO-d₆) δ 7.29 (d, J=10.1 Hz, 1H), 7.03 (d, J=3.5 Hz, 1H), 6.84 (d, J=8.3 Hz, 2H), 6.66 (d, J=3.5 Hz, 1H), 6.49-6.41 (m, 2H), 6.25 (dd, J=10.1, 1.9 Hz, 1H), 6.08 (s, 1H), 5.73 (s, 1H), 5.46-5.40 (m, 1H), 5.11 (t, J=5.9 Hz, 1H), 4.91 (s, 2H), 4.89-4.85 (m, 1H), 4.49 (dd, J=19.5, 6.4 Hz, 1H), 4.22-4.12 (m, 2H), 3.88 (s, 2H), 3.32 (s, 1H), 2.66-2.59 (m, 1H), 2.41-2.34 (m, 1H), 2.21-2.09 (m, 1H), 2.04-1.96 (m, 1H), 1.87-1.78 (m, 1H), 1.68-1.57 (m, 3H), 1.49 (s, 3H), 1.43-1.31 (m, 1H), 0.84 (s, 3H).

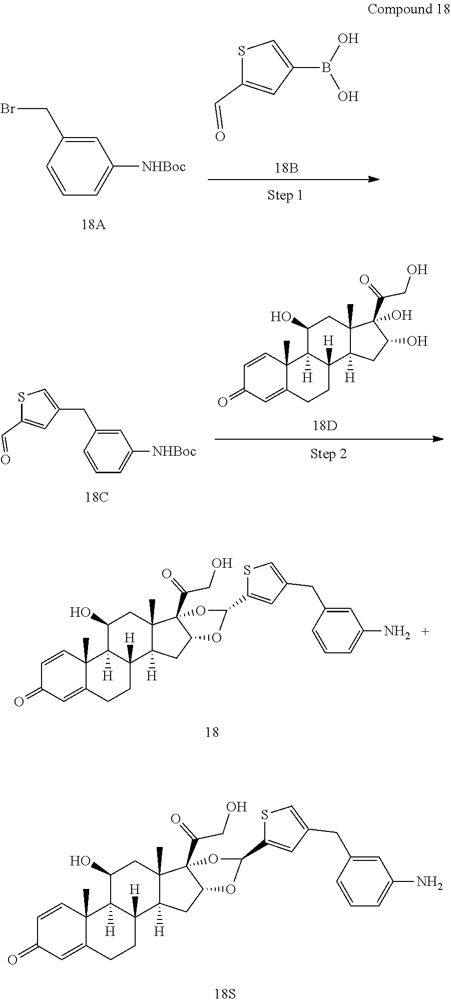

Step 1 Compound 18A (1.01 g, 3.53 mmol) and compound 18B (0.50 g, 3.21 mmol) were dissolved in THF (15.0 mL). Sodium carbonate (1.36 g, 12.8 mmol) was dissolved in water (1.50 mL), and the resulting solution was added to the reaction solution. The mixed solution was purged three times with nitrogen and stirred at room temperature for 15 min. Pd(PPh₃)₄ (59.1 mg, 160 μmol) was added to the reaction solution, and the resulting solution was purged three times with nitrogen, heated to 90° C. and stirred for 16 h. After the starting materials were completely consumed as detected by LCMS, the mixture was cooled to room temperature and poured into water (15.0 mL). The resulting mixture was extracted with ethyl acetate (15.0 mL, 10.0 mL). The organic phases were combined, washed with a saturated aqueous sodium chloride solution (10.0 mL), dried over anhydrous sodium sulfate, filtered and concentrated to give a crude product, which was purified by reversed-phase preparative chromatography [water (NH₄HCO₃)-acetonitrile] to give a white solid 18C (450 mg, yield: 44.2%). MS-ESI: m/z 262.1 [M+H-C₄H₈]^{+.}

Step 2 Two reactions were set up in parallel. Compound 18C (0.20 g, 630 μmol) and compound 18D (237 mg, 630 μmol) were dissolved in acetonitrile (2.00 mL), anhydrous magnesium sulfate (379 mg, 3.15 mmol) was added, and the mixed solution was cooled to -10° C. TfOH (540 mg, 2.52 mmol, 317 μL) was added dropwise, and the resulting solution was slowly heated to 20° C. and stirred for 12 h. After there was 82% of the product generated as detected by LCMS, the two reaction solutions were combined, quenched with a saturated aqueous sodium bicarbonate solution (8.00 mL) and extracted with ethyl acetate (10.0 mL, 8.00 mL, 6.00 mL). The organic phases were combined, washed with a saturated aqueous sodium chloride solution (6.00 mL), dried over anhydrous sodium sulfate, filtered and concentrated to give a crude product, which was purified by reversed-phase preparative chromatography [water (NH₄HCO₃)-acetonitrile] to give a white solid 18 (128 mg, yield: 17.7%) and compound 18S (26.3 mg, yield: 3.63%).

18:

MS-ESI: m/z 576.1 [M+H]^{+.}

¹H NMR (400 MHz, DMSO-d₆) δ 7.32 (d, J=10.1 Hz, 1H), 7.18 (s, 1H), 7.03 (d, J=1.5 Hz, 1H), 6.90 (dd, J=8.6, 7.4 Hz, 1H), 6.40-6.31 (m, 3H), 6.18 (dd, J=10.0, 1.9 Hz, 1H), 5.96 (s, 1H), 5.79 (s, 1H), 5.06 (t, J=5.9 Hz, 1H), 4.95 (s, 2H), 4.89 (d, J=4.8 Hz, 1H), 4.81 (d, J=3.4 Hz, 1H), 4.49 (dd, J=19.4, 6.3 Hz, 1H), 4.33-4.29 (m, 1H), 4.16 (dd, J=19.5, 5.6 Hz, 1H), 3.68 (s, 2H), 2.62-2.54 (m, 1H), 2.37-2.31 (m, 1H), 2.19-2.03 (m, 2H), 1.78-1.59 (m, 5H), 1.40 (s, 3H), 1.06-0.94 (m, 1H), 0.90 (dd, J=10.7, 3.4 Hz, 1H), 0.85 (s, 3H).

### 18S:

MS-ESI: m/z 576.1 [M+H]^{+.}

¹H NMR (400 MHz, DMSO-d₆) δ 7.30 (d, J=10.1 Hz, 1H), 7.19 (s, 1H), 6.96-6.85 (m, 2H), 6.39-6.28 (m, 4H), 6.16 (d, J=10.2 Hz, 1H), 5.93 (s, 1H), 5.21 (d, J=7.2 Hz, 1H), 5.07-5.00 (m, 1H), 4.95 (s, 2H), 4.81-4.73 (m, 1H), 4.43-4.32 (m, 1H), 4.32-4.24 (m, 1H), 4.11-3.99 (m, 1H), 3.67 (s, 2H), 2.35-2.31 (m, 1H), 2.06-1.95 (m, 2H), 1.86-1.78 (m, 2H), 1.76-1.64 (m, 4H), 1.38 (s, 3H), 1.21-1.12 (m, 1H), 1.06-0.99 (m, 1H), 0.86 (s, 3H).

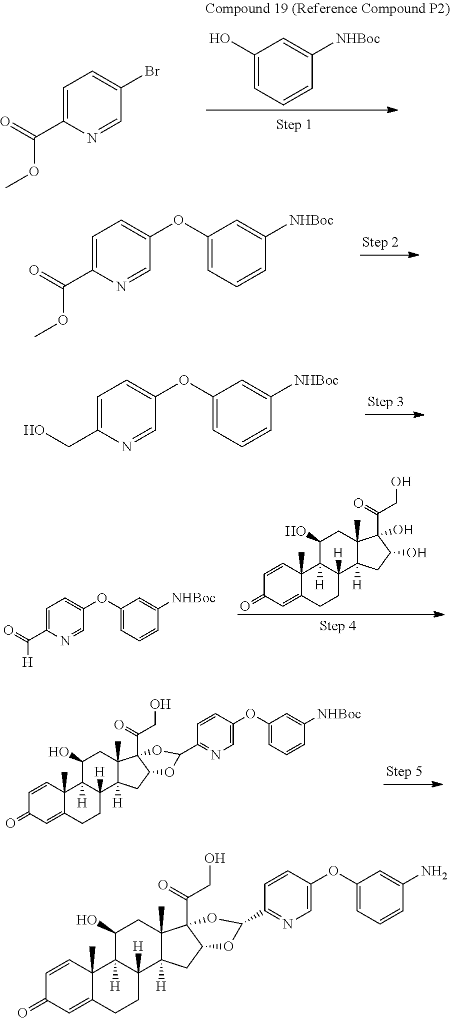

### Step 1: Synthesis of methyl 5-(3-((tert-butoxycarbonyl)amino)phenoxy)picolinate

Compound tert-butyl (3-hydroxyphenyl)carbamate (2.5 g, 12.0 mmol, 1 eq) and compound methyl 5-bromopicolinate (2.84 g, 13.2 mmol, 1.1 eq) were added to a three-necked flask and dissolved in dry DMF (50 mL). Cuprous iodide (0.25 g, 1.32 mmol, 0.1 eq) and L-proline (0.35 g, 1.32 mmol, 0.1 eq) were added, and the mixed solution was heated to 100° C. and reacted overnight under nitrogen atmosphere. After the reaction was completed as detected by TLC (PE:EA=20:1), 1 N hydrochloric acid was added to quench the reaction, and the reaction solution was extracted with dichloromethane, washed three times with 1 N hydrochloric acid, concentrated by rotary evaporation and purified by silica gel column chromatography (PE:EA=15:1) to give a white solid (4.13 g, yield: 91%).

### Step 2: Synthesis of tert-butyl (3-(((6-(hydroxymethyl)pyridin-3-yl)oxy)phenyl)carbamate

The compound methyl 5-(3-((tert-butoxycarbonyl)amino)phenoxy)picolinate (1.5 g, 4.36 mmol) was dissolved in THF (10 mL), and the resulting solution was purged with nitrogen and cooled to −20° C. Lithium aluminum hydride (333 mg, 8.72 mmol) was added slowly in batches. After 30 min, the reaction was completed as detected by TLC (PE/EA=3/1), and the reaction solution was slowly added to 1 N hydrochloric acid (30 mL) to quench the reaction. The mixed solution was extracted with ethyl acetate (20 mL), and the organic phase was washed with a saturated sodium chloride solution (30 mL), dried over anhydrous sodium sulfate and concentrated under reduced pressure to give a light yellow oil product (1.2 g, yield: 87%), which was used directly in the next step.

### Step 3: Synthesis of tert-butyl (3-(((6-formylpyridin-3-yl)oxy)phenyl)carbamate

tert-Butyl (3-(((6-(hydroxymethyl)pyridin-3-yl)oxy)phenyl)carbamate (1.1 g, 3.48 mmol) was added to dichloromethane (20 mL), and the mixed solution was cooled to 0° C. Dess-Martin periodinane (1,1,1-triacetoxy-1,1-dihydro-1,2-benzotriazol-3(1H)-one) (3.61 g, 8.7 mmol) was added. After the mixed solution was reacted for 3 h, the reaction was completed as detected by TLC (petroleum ether/ethyl acetate=1/1), the reaction solution was diluted with dichloromethane (30 mL), and a saturated NaCO3 solution (50 mL) was added for washing. The organic phase was washed with saturated NaCl, dried over anhydrous Na₂SO₄ and purified by column chromatography (petroleum ether: ethyl acetate=20:1) to give a light yellow solid (553 mg, yield: 51%).

Step 4: Synthesis of tert-butyl (3-((6-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,l1a,12,12a,12b-dodecahydro-1H-naphtho[2′,1′:4,5]indeno[1,2-d][1,3]dioxol-10-yl)pyridin-3-yloxy)phenyl)carbamate

The compound tert-butyl (3-(((6-formylpyridin-3-yl)oxy)phenyl)carbamate (300 mg, 0.96 mmol), (8S,9S,10R,11S,13S,14S,16R,17S)-11,16,17-trihydroxy-17-(2-hydroxyacetyl)-10,13-dimethyl-6,7,8,9,10,11,12,13,14,15,16,17-dodecahydro-3H-cyclopenta[a]phenanthrylanthracen-3-one (361 mg, 0.96 mmol) and MgSO₄ (600 mg) were added to a 25 mL three-necked flask, acetonitrile (5 mL) was added for dissolution, and the mixed solution was stirred for 10 min. TfOH (307 mg, 2.04 mmol) was added. After 2 h, the reaction was completed as detected by TLC (PE/EA=1/1), the reaction solution was added to EA (40 mL), and the resulting solution was washed twice with water (50 mL×2), washed with a saturated NaCO₃ solution (50 mL) and saturated NaCl, and dried over Na₂SO₄ to give a light yellow solid (402 mg, yield: 63%).

### Step 6: Synthesis of (6aR,6bS,7S,8aS,8bS,10R,11aR,12aS,12bS)-10-(5-(3-aminophenoxy)pyridin-2-yl)-7-hydroxy-8b-(2-hydroxyacetyl)-6a,8a-dimethyl-1,2,6a,6b,7,8,8a,8b,11a,12,12a,12b-dodecahydro-4H-naphtho[2′,1′:4,5]indeno[1,2-d][1,3]dioxol-4-one

The compound tert-butyl (3-((6-((6aR,6bS,7S,8aS,8bS,11aR,12aS,12bS)-7-hydroxy-8b-(2-hydroxyacetyl)-6a,8a-dimethyl-4-oxo-2,4,6a,6b,7,8,8a,8b,11a,12,12a,12b-dodecahydro-1H-naphtho[2′,1′:4,5]indeno[1,2-d][1,3]dioxol-10-yl)pyridin-3-yloxy)phenyl)carbamate (210 mg, 0.31 mmol) was dissolved in dry dichloromethane (10 mL). Trifluoroacetic acid (1 mL) was added dropwise, and the mixed solution was cooled to 5° C. and stirred for 2 h. After the reaction was completed as detected by TLC (PE:EA=1:1), the reaction solution was diluted with DCM and concentrated by rotary evaporation to remove the solvent. The crude product was purified by preparative HPLC to give a white solid (51.4 mg, yield: 29%). LC-MS [M+H]=573.0.

¹H NMR (400 MHz, DMSO) δ 8.31 (d, J=2.4 Hz, 1H), 7.64 (d, J=8.4 Hz, 1H), 7.45-7.55 (m, 1H), 7.33 (d, J=10 Hz, 1H), 7.53 (t, J=8.0 Hz, 1H), 6.40-6.50 (m, 1H), 6.20-6.30 (m, 3H), 5.94 (s, 1H), 5.30-5.40 (m, 3H), (s, 2H), 4.87 (d, J=3.6 Hz, 1H), 4.30-4.40 (m, 2H), 4.00-4.20 (m, 3H), 2.30-2.40 (m, 2H), 1.90-2.30 (m, 7H), 1.60-1.70 (m, 1H), 1.47 (s, 3H), 1.41 (s, 3H), 1.20-1.30 (m, 1H), 0.90-1.00 (m, 3H).

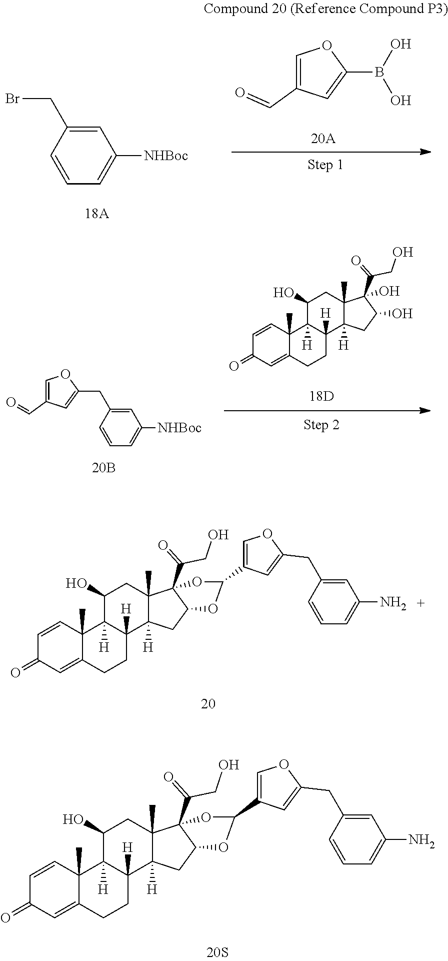

Step 1 Compound 18A (112 mg, 393 μmol) and compound 20A (50 mg, 359 μmol) were dissolved in DMF (1 mL). Potassium carbonate (98.8 mg, 714 μmol) was added to the reaction solution. The resulting solution was purged three times with nitrogen and stirred for 15 min. Pd(PPh₃)₄ (13.2 mg, 35.7 μmol) was added to the reaction solution, and the resulting solution was purged three times with nitrogen, heated to 90° C. and stirred for 16 h. After the starting materials were completely consumed as detected by LCMS, the mixture was cooled to room temperature and poured into water (1 mL). The resulting mixture was extracted with ethyl acetate (1 mL×3). The organic phases were combined, washed with saturated brine (1 mL), dried over anhydrous sodium sulfate, filtered and concentrated to give a crude product, which was purified by reversed-phase preparative chromatography [water (NH₄HCO₃)-acetonitrile] to give a white solid, compound 20B (45 mg, yield: 41.7%). MS-ESI: m/z 246.2 [M+H-C4H₈]^{+.}

Step 2 Compound 20B (50.0 mg, 165 μmol) and compound 18D (62.4 mg, 165 μmol) were dissolved in acetonitrile (0.5 mL), anhydrous magnesium sulfate (99.8 mg, 830 μmol) was added, and TfOH (99.6 mg, 663 μmol, 58.5 μL) was added dropwise at −10° C. The mixed solution was gradually heated to 20° C. and stirred for 16 h. After there were 35% of the product as detected by LCMS, the reaction solution was quenched with a saturated aqueous sodium bicarbonate solution (10.0 mL) and extracted with ethyl acetate (10.0 mL, 8.00 mL, 6.00 mL). The organic phases were combined, washed with a saturated aqueous sodium chloride solution (5.00 mL), dried over anhydrous sodium sulfate, filtered and concentrated to give a crude product, which was purified by reversed-phase preparative chromatography [water (TFA)-acetonitrile] to give a white solid, compound 20 (3 mg, yield: 3.23%) and compound 20S (3 mg, yield: 3.23%).

20:

MS-ESI: m/z 560.1 [M+H]^{+.}

¹H NMR (400 MHz, Methanol-d₄) δ 7.53-7.26 (m, 2H), 7.26-7.04 (m, 3H), 6.53-6.43 (m, 1H), 6.28 (d, J=10.2 Hz, 1H), 6.13 (s, 1H), 6.02 (s, 1H), 5.57 (s, 1H), 5.01 (d, J=5.3 Hz, 1H), 4.56 (d, J=19.2 Hz, 1H), 4.38 (s, 1H), 4.27 (d, J=19.5 Hz, 1H), 4.00 (s, 2H), 2.74-2.59 (m, 1H), 2.44-2.30 (m, 1H), 2.30-2.18 (m, 1H), 2.18-2.06 (m, 1H), 1.97-1.83 (m, 2H), 1.83-1.65 (m, 3H), 1.49 (s, 3H), 1.10-0.89 (m, 5H).

### 20S:

MS-ESI: m/z 560.1 [M+H]^{+.}

¹H NMR (400 MHz, Methanol-d₄) δ 7.48-7.40 (m, 2H), 7.35-7.28 (m, 1H), 7.25-7.16 (m, 2H), 6.30 (d, J=3.2 Hz, 1H), 6.25 (dd, J=10.1, 1.9 Hz, 1H), 6.11-6.06 (m, 2H), 5.99 (s, 1H), 5.24 (t, J=3.4 Hz, 1H), 4.40 (q, J=3.2 Hz, 1H), 4.17-4.05 (m, 2H), 3.99 (s, 2H), 2.65 (td, J=13.7, 5.6 Hz, 1H), 2.41-2.32 (m, 1H), 2.28-2.07 (m, 2H), 1.95 (dd, J=13.7, 3.5 Hz, 1H), 1.84-1.75 (m, 2H), 1.73-1.60 (m, 2H), 1.48 (s, 3H), 1.17 (qd, J=13.0, 4.8 Hz, 1H), 1.07 (dd, J=11.2, 3.5 Hz, 1H), 0.97 (s, 3H).

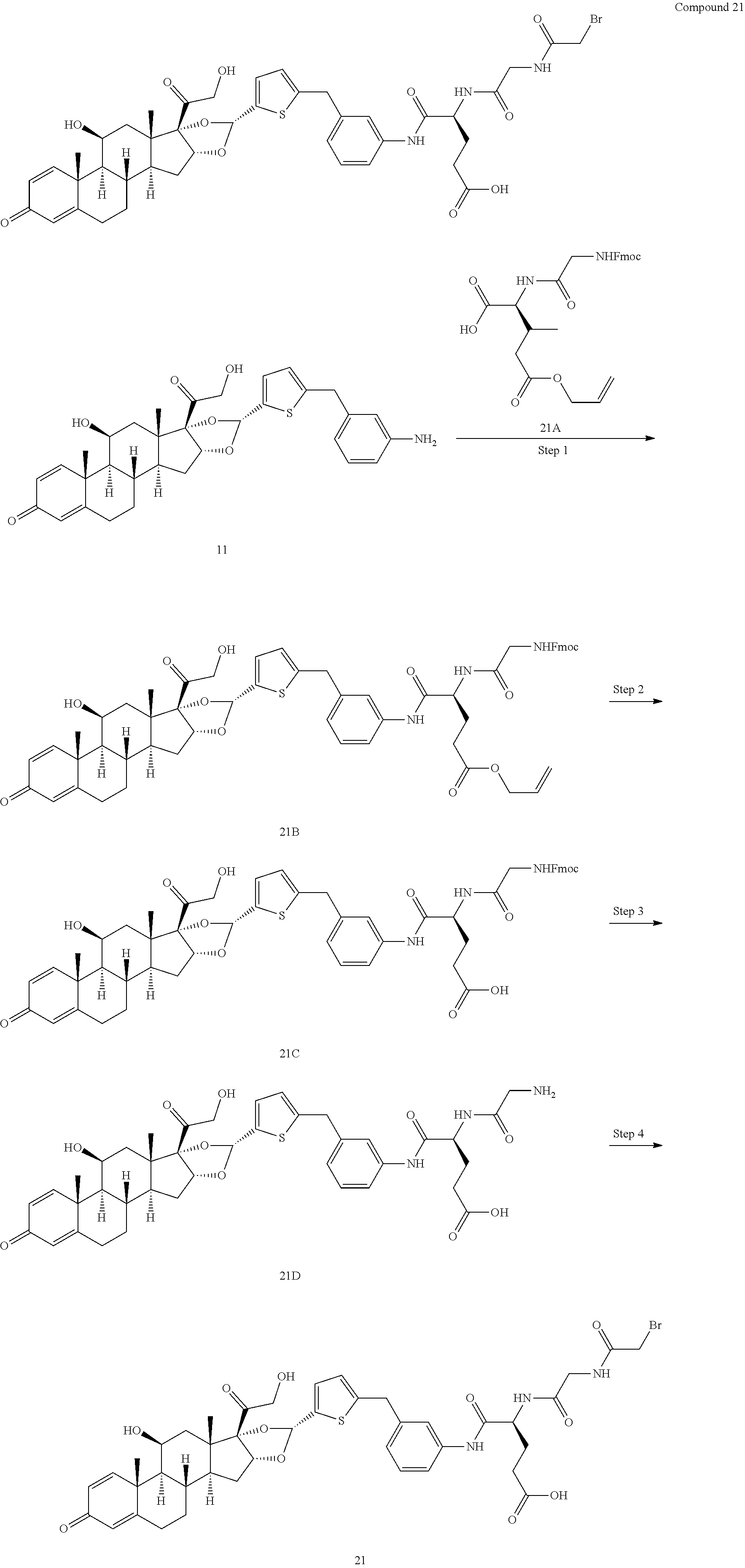

Step 1 Compound 11 (2 g, 3.48 mmol), 21A (1.63 g, 3.48 mmol) and HATU (1.59 g, 4.18 mmol) were added to a 100 mL three-necked flask, DMF (30 mL) was added for dissolution, and 2,6-lutidine (1.12 g, 10.44 mmol) was slowly added. After the starting materials were completely consumed as detected by LCMS, the reaction solution was slowly added dropwise to water (300 mL) and filtered to give a product, which was dried by oil pump under vacuum to give a white solid 21B (3.5 g, yield: 98%). MS-ESI: m/z 1024.4 [M+H]^{+.}

Step 2 Compound 19B (1.0 g, 1.0 mmol, 1.0 eq) and Pd(PPh₃)₄(392 mg, 0.339 mmol, 0.35 eq) were added to a 50 mL three-necked flask, and DCM (30 mL) was added for dissolution. Under nitrogen atmosphere, NMMP (868 mg, 8.6 mmol, 8.8 eq) was slowly added. After the mixed solution was reacted for 0.5 h, the reaction was completed as detected by LCMS, mixed with silica gel, concentrated and purified by column chromatography (DCM in MeOH from 0% to 15%) to give a yellow solid 21C (0.88 g, yield: 92%). MS-ESI: m/z 984.4 [M+H]^{+.}

Step 3 Compound 21C (700 mg, 0.71 mmol) was dissolved in DMF (10 mL) and DEA (2 mL), and the mixed solution was reacted at room temperature for 15 min. After the reaction was completed as detected by LCMS, the reaction solution was purified by reversed-phase column chromatography (ACN in water from 20% to 50%) to give a yellow solid 21D (300 mg, yield: 55%). MS-ESI: m/z 762.3 [M+H]^{+.}

Step 4 Compound 21D (100 mg, 0.13 mmol) was dissolved in THF (5 mL) and water (0.5 mL), and 2,6-lutidine (139 mg, 1.3 mmol) was added at room temperature. Bromoacetyl bromide (130 mg, 0.65 mmol) was dissolved in THF (1 mL), and the resulting solution was slowly added to the reaction solution. The mixed solution was reacted at room temperature for 15 min. After the reaction was completed as detected by LCMS, the reaction solution was purified by prep-HPLC (ACN in 0.1% TFA) to give a white solid 21 (19.4 mg, yield: 17%).

MS-ESI: m/z 882.2 [M+H]^{+.}

¹H NMR (400 MHz, DMSO-d₆) δ 12.14 (brs, 1H), 9.92 (s, 1H), 8.52 (t, J=5.6 Hz, 1H), 8.25 (d, J=7.8 Hz, 1H), 7.51-7.42 (m, 2H), 7.31 (d, J=10.1 Hz, 1H), 7.23-7.14 (m, 1H), 7.08 (d, J=3.5 Hz, 1H), 6.96-6.89 (m, 1H), 6.73 (d, J=3.5 Hz, 1H), 6.18 (dd, J=10.1, 1.9 Hz, 1H), 5.97 (s, 1H), 5.73 (s, 1H), 4.88 (d, J=4.8 Hz, 1H), 4.79 (s, 1H), 4.49 (d, J=19.5 Hz, 1H), 4.44-4.35 (m, 1H), 4.29 (s, 1H), 4.15 (d, J=19.5 Hz, 1H), 4.07 (s, 2H), 3.94 (s, 2H), 3.86-3.75 (m, 2H), 2.59-2.52 (m, 1H), 2.38-2.17 (m, 3H), 2.17-1.91 (m, 3H), 1.89-1.78 (m, 1H), 1.78-1.54 (m, 5H), 1.39 (s, 3H), 1.01-0.91 (m, 1H), 0.88 (dd, J=11.2, 3.5 Hz, 1H), 0.84 (s, 3H).

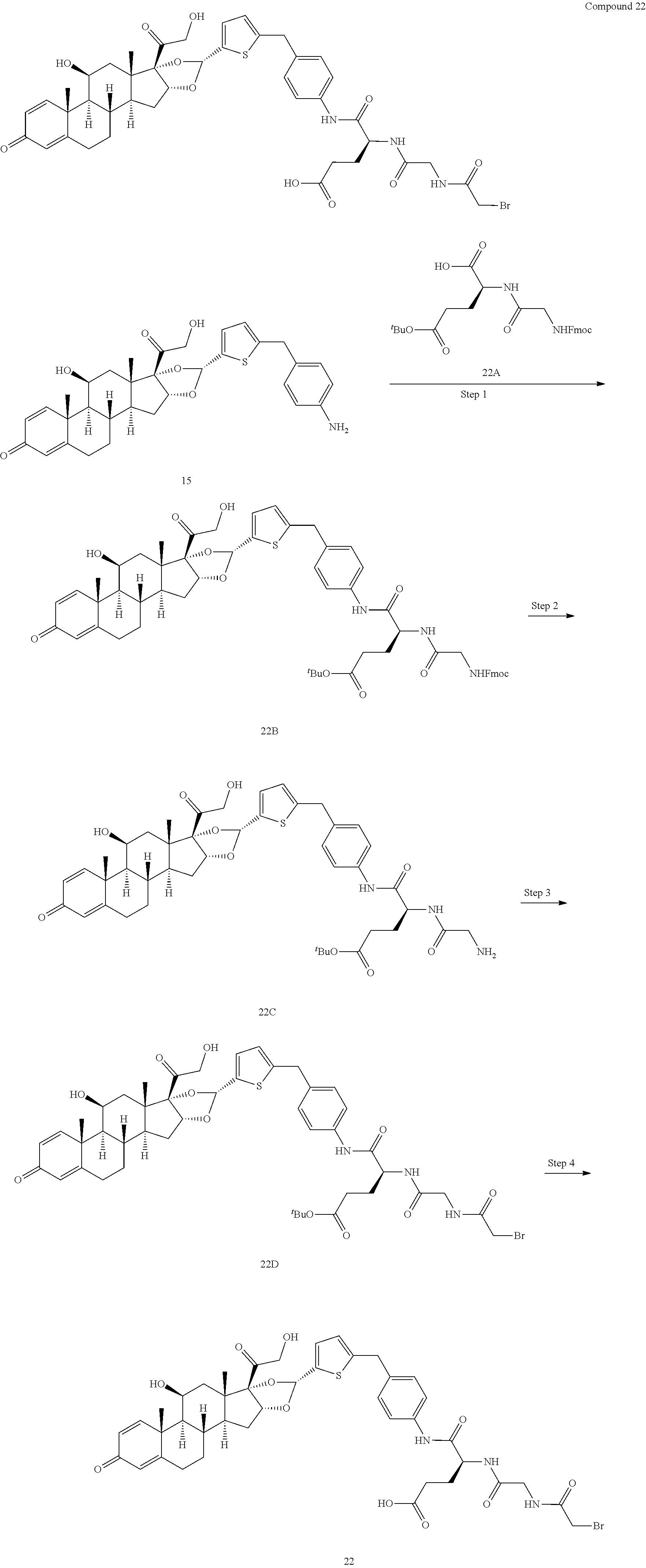

Step 1 Compound 15 (350 mg, 0.6 mmol) and compound 22A (289 mg, 0.6 mmol) were added to a 25 mL three-necked flask, and DMF (8 mL) was added. Under nitrogen atmosphere, 2,6-lutidine (196 mg, 1.8 mmol) was added in an ice-water bath, and the mixed solution was stirred for 10 min. HATU (301 mg, 0.79 mmol) was added dropwise to DMF solution (1 mL). After addition, the resulting solution was heated to room temperature and stirred for 1 h. After the reaction was completed as detected by LCMS, the reaction solution was slowly added dropwise to water (100 mL), a large amount of solid was precipitated, and the mixture was stirred for 30 min and filtered. The solid was lyophilized to give a white solid 22B (525 mg, yield: 83%). MS-ESI: m/z 1040.4 [M+H]^{+.}

Step 2 Compound 22B (200 mg, 0.192 mmol) and DCM (4 mL) were added to a 25 mL single-neck flask, and the mixed solution was stirred for complete dissolution under nitrogen atmosphere. DEA (0.57 mL) was added dropwise, and after addition, the resulting solution was heated to room temperature and reacted for 4 h. After the reaction was completed as detected by LCMS, the reaction solution was concentrated to dryness and purified by reversed-phase preparative chromatography to give a white solid 22C (70 mg, yield: 44%). MS-ESI: m/z 818.4 [M+H]⁺.

Step 3 Compound 22C (70 mg, 0.086 mmol) was dissolved in THF (5 mL) and water (0.5 mL), 2,6-lutidine (46 mg, 0.43 mmol) was added in an ice-water bath, and the resulting solution was stirred for 5 min. Bromoacetyl bromide (86 mg, 0.43 mmol) was added, and after addition, the mixed solution was reacted in an ice-water bath for 10 min. After the reaction was completed as detected by LCMS, the reaction solution was added to water, the pH was adjusted to 1-2 with TFA, and the product was extracted with EA (20 mL). The organic phase was washed with water (10 mL×2), dried over anhydrous sodium sulfate, concentrated to a small volume and directly used in the next step with negligible yield of crude product. MS-ESI: m/z 938.3 [M+H]^{+.}

Step 4 Under nitrogen atmosphere, the organic phase from the previous step (3 mL) was added to TFA (1.5 mL) at room temperature. The resulting solution was stirred at room temperature for 4 h. After the reaction was completed as detected by LCMS, the reaction solution was directly purified by reversed-phase preparative chromatography and lyophilized to give a white solid 22 (6.5 mg, two-step yield: 9%).

MS-ESI: m/z 882.2 [M+H]^{+.}

¹H NMR (400 MHz, DMSO-d₆) δ 12.14 (brs, 1H), 9.91 (s, 1H), 8.52 (t, J=5.6 Hz, 1H), 8.27 (d, J=7.9 Hz, 1H), 7.52 (d, J=8.6 Hz, 2H), 7.32 (d, J=10.1 Hz, 1H), 7.15 (d, J=8.6 Hz, 2H), 7.07 (s, 1H), 6.70 (d, J=3.5 Hz, 1H), 6.19 (dd, J=10.1, 1.9 Hz, 1H), 5.99 (s, 1H), 5.72 (s, 1H), 4.88 (d, J=4.7 Hz, 1H), 4.80 (s, 1H), 4.49 (d, J=19.5 Hz, 1H), 4.45-4.37 (m, 1H), 4.30 (s, 1H), 4.20-4.11 (m, 1H), 4.11-3.98 (m, 2H), 3.94 (s, 2H), 3.81 (dd, J=5.7, 1.6 Hz, 2H), 2.60-2.53 (m, 1H), 2.38-2.24 (m, 3H), 2.17-1.93 (m, 3H), 1.89-1.77 (m, 1H), 1.77-1.70 (m, 2H), 1.70-1.57 (m, 3H), 1.39 (s, 3H), 1.05-0.92 (m, 1H), 0.91 (dd, J=11.1, 3.4 Hz, 1H), 0.84 (s, 3H).

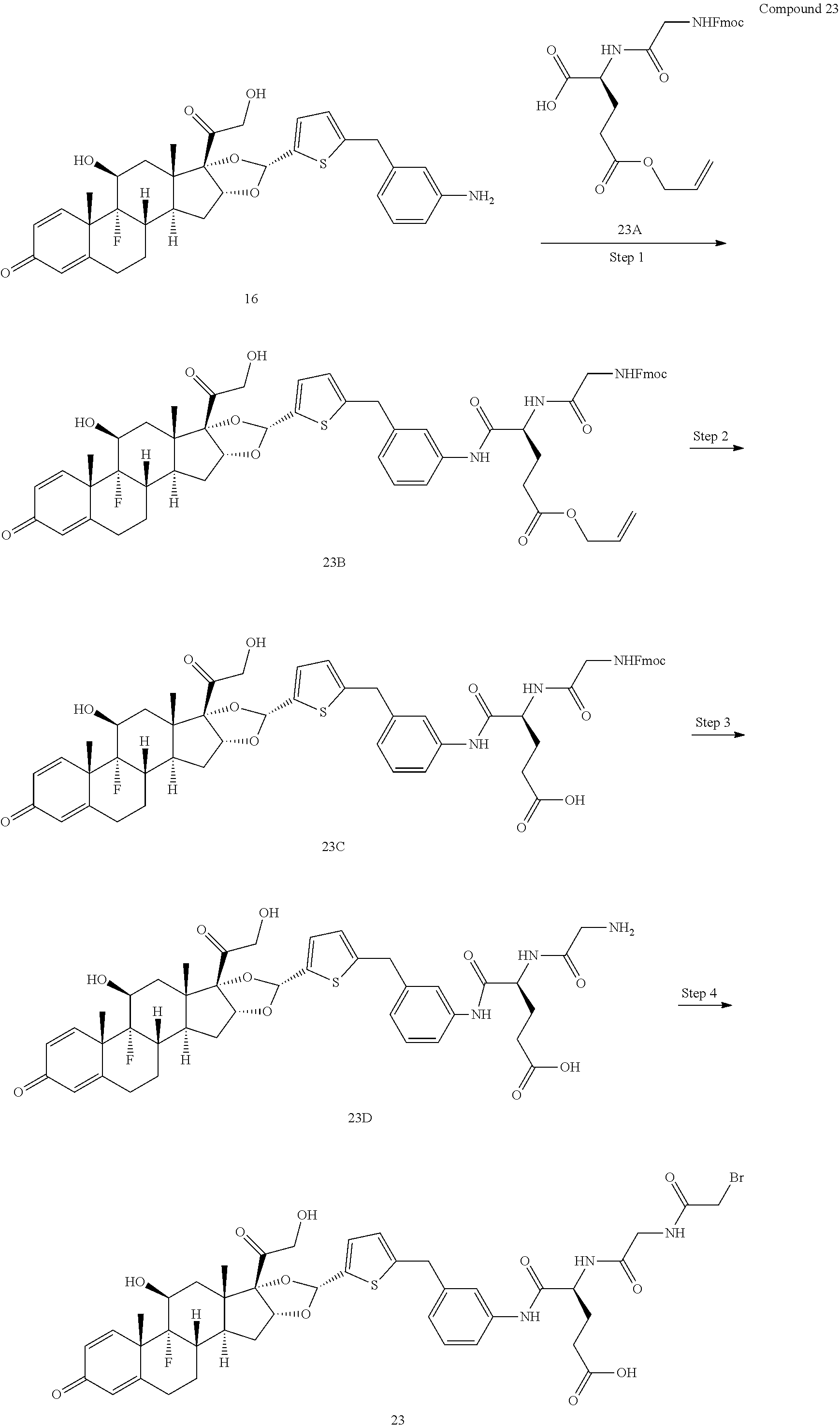

Step 1 Compound 16 (540 mg, 0.91 mmol), compound 23A (353 mg, 0.76 mmol) and 2,6-lutidine (121 mg, 1.14 mmol) were added to a three-necked flask (25 mL), DMF (4 mL) was added for dissolution, and HATU (316 mg, 0.833 mmol) was added. The mixed solution was stirred for 17 h. After the reaction was completed as detected by LCMS, the reaction solution was slowly added to water (80 mL), and a large amount of yellow solid was precipitated. The resulting mixture was filtered, and the filter cake was washed with water (80 mL) and lyophilized to give a yellow solid 23B (700 mg, yield: 88%). MS-ESI: m/z 1042.4 [M+H]^{+.}

Step 2 Compound 23B (700 mg, 0.67 mmol), N-methylmorpholine (679 mg, 6.7 mmol) and Pd(PPh₃)₄ (310 mg, 0.27 mmol) were added to a 25 mL three-necked flask, and THF (5 mL) was added for dissolution. The mixed solution was purged with nitrogen, cooled to 0° C. and reacted for 2 h. After the reaction was completed as detected by LCMS, the reaction solution was directly mixed with silica gel and purified by normal phase column chromatography (DCM/MeOH=80/20) to give a yellow powder 23C (450 mg, yield: 66%). MS-ESI: m/z 1002.3 [M+H]^{+.}

Step 3 Compound 23C (450 mg) was dissolved in DCM (3 mL), diethylamine (1.5 mL) was added, and the mixed solution was stirred at room temperature for 2 h. After the reaction was completed as detected by LCMS, the reaction solution was added dropwise to PE (60 mL), and a large amount of yellow solid was precipitated. After the resulting mixture was left to stand for 5 min, the reaction solution was filtered to give a yellow powder (200 mg), which was purified by reversed-phase preparative chromatography to give a white solid 23D (15 mg, yield: 4%). MS-ESI: m/z 780.3 [M+H]⁺.

Step 4 Compound 23D (15 mg, 0.019 mmol) was dissolved in THF (0.8 mL) and H₂O (0.2 mL). The resulting solution was cooled to 5° C. in an ice-water bath, and 2,6-lutidine (20.5 mg, 0.19 mmol) and bromoacetyl bromide (15.8 mg, 0.07 mmol) were added. The mixed solution was stirred for 5 min. After the reaction was completed as detected by LCMS, the reaction solution was purified by reversed-phase preparative chromatography to give a white powder 23 (6 mg, yield: 34%). MS-ESI: m/z 900.2 [M+H]^{+.}

¹H NMR (400 MHz, DMSO-d₆) δ 9.93 (s, 1H), 8.55-8.48 (m, 1H), 8.24 (d, J=7.8 Hz, 1H), 7.49 (d, J=8.2 Hz, 1H), 7.44 (s, 1H), 7.29 (d, J=10.1 Hz, 1H), 7.23-7.17 (m, 1H), 7.09-7.06 (m, 1H), 6.97-6.90 (m, 1H), 6.72 (d, J=3.5 Hz, 1H), 6.24 (dd, J=10.1, 1.9 Hz, 1H), 6.06 (s, 1H), 5.74 (s, 1H), 5.44-5.39 (m, 1H), 4.91-4.87 (m, 1H), 4.50 (d, J=19.4 Hz, 1H), 4.42-4.36 (m, 1H), 4.17 (d, J=19.5 Hz, 3H), 4.07 (s, 2H), 3.94 (s, 2H), 3.83-3.78 (m, 2H), 2.40-2.09 (m, 2H), 2.05-1.75 (m, 3H), 1.69-1.57 (m, 3H), 1.49 (s, 3H), 1.45-1.28 (m, 1H), 0.84 (s, 3H).

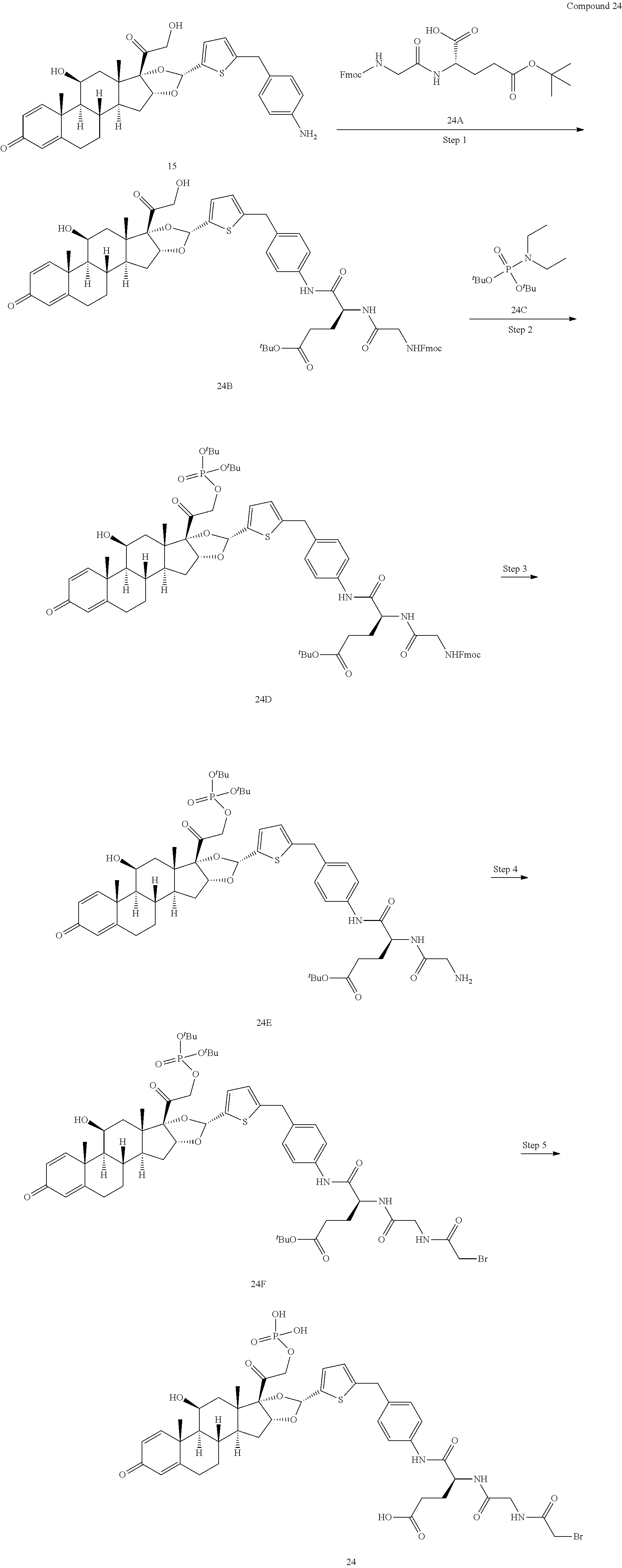

Step 1 Compound 24A (4.53 g, 9.38 mmol) was dissolved in pyridine (36.0 mL) and DMF (36.0 mL), EDCt (3.60 g, 18.8 mmol) was added, and compound 15 (4.53 g, 9.38 mmol) was added to the reaction solution at 0° C. The resulting solution returned to room temperature and stirred for 1 h. After the starting materials were completely consumed as detected by LCMS, the mixture was poured into water (118 mL). The resulting mixture was diluted with ethyl acetate (118 mL) and extracted with ethyl acetate (100 m, 50.0 m). The organic phases were combined, washed with a saturated aqueous sodium chloride solution (30.0 mL), dried over anhydrous sodium sulfate, filtered and concentrated to give a yellow solid 24B (crude, 6.50 g, with negligible yield of crude product). MS-ESI: m/z 1040.4 [M+H]^{+.}

Step 2 Compound 24B (6.30 g, 6.06 mmol), tetrazole (4.24 g, 60.6 mmol, 5.37 mL) and compound 24C (9.06 g, 36.3 mmol) were dissolved in DMF (44.0 mL). The mixed solution was stirred at 20° C. for 2.5 h, and the internal temperature was reduced to 0° C. H₂O₂ (3.78 g, 33.3 mmol, 3.20 mL, 30% purity) was added to the reaction solution at 0° C., and the resulting solution returned to room temperature and stirred for 1 h. After the starting materials were completely consumed as detected by LCMS, the mixture was poured into a saturated aqueous sodium sulfite solution (44.0 mL). The resulting mixture was diluted with water (88.0 mL) and extracted with ethyl acetate (100 mL, 50.0 mL, 30.0 mL). The organic phases were combined, washed with a saturated aqueous sodium chloride solution (20.0 mL), dried over anhydrous sodium sulfate, filtered and concentrated to give a yellow solid 24D (crude, 7.46 g, with negligible yield of crude product). MS-ESI: m/z 1232.5 [M+H]^{+.}

Step 3 Compound 24D (7.16 g, 5.81 mmol) and piperidine (4.20 g, 49.4 mmol, 4.88 mL) were dissolved in acetonitrile (40.0 mL), and the mixed solution was stirred at 20° C. for 1 h. After the starting materials were completely consumed as detected by LCMS, the reaction solution was concentrated under reduced pressure to remove acetonitrile. The residue after concentration was slurried with petroleum ether for 2 h and filtered, and the filter cake was dried under vacuum to give a yellow solid 24E (6.37 g, yield: 77.7%), which was confirmed by LCMS (LCMS_ET54964-22-p1b1). MS-ESI: m/z 1010.5 [M+H]^{+.}

Step 4 Bromoacetic acid (1.61 g, 11.6 mmol, 837 μL) and EEDQ (2.87 g, 11.6 mmol) were dissolved in DMF (41.0 mL), the mixed solution was stirred at 20° C. for 1 h, and compound 24E (5.87 g, 5.81 mmol) was added to the reaction solution at 20° C. The resulting solution was reacted at 20° C. for 2.5 h. After the starting materials were completely consumed as detected by LCMS, the reaction solution was diluted with dichloromethane (220 mL), washed twice with 1 N aqueous hydrobromic acid solution (160 mL×2), washed once with a saturated aqueous sodium bicarbonate solution (110 mL), washed once with a saturated aqueous sodium chloride solution (50.0 mL), dried over anhydrous sodium sulfate, filtered and concentrated to give a yellow oily liquid 24F (crude, 6.57 g, with negligible yield of crude product). MS-ESI: m/z 1130.3 [M+H]^{+.}

Step 5 Compound 24F (6.57 g, 5.81 mmol) was dissolved in dichloromethane (50.0 mL), and TFA (25.0 mL) was added to the reaction solution at 20° C. The resulting solution was stirred for 1 h. After the starting materials were completely consumed as detected by LCMS, the reaction solution was concentrated under reduced pressure to give a crude product, which was purified by reversed-phase preparative chromatography [water (NH₄HCO₃)-acetonitrile] to give a yellow solid 24 (355 mg, yield: 6.36%). MS-ESI: m/z 961.9 [M+H]^{+.}

¹H NMR (400 MHz, DMSO-d₆) δ 11.65 (brs, 1H), 9.93 (s, 1H), 8.54 (t, J=5.6 Hz, 1H), 8.32-8.24 (m, 1H), 7.52 (d, J=8.5 Hz, 2H), 7.32 (d, J=10.1 Hz, 1H), 7.15 (d, J=8.6 Hz, 2H), 7.06 (d, J=3.6 Hz, 1H), 6.70 (d, J=3.5 Hz, 1H), 6.19 (dd, J=10.1, 1.9 Hz, 1H), 5.99 (s, 1H), 5.81 (s, 1H), 4.94-4.84 (m, 2H), 4.56 (dd, J=18.2, 7.9 Hz, 1H), 4.47-4.37 (m, 1H), 4.34-4.28 (m, 1H), 4.09-3.97 (m, 2H), 3.94 (s, 2H), 3.87-3.75 (m, 2H), 2.59-2.52 (m, 1H), 2.39-2.18 (m, 3H), 2.18-2.06 (m, 1H), 2.06-1.92 (m, 2H), 1.90-1.79 (m, 1H), 1.75 (s, 2H), 1.71-1.54 (m, 3H), 1.39 (s, 3H), 0.99-0.88 (m, 2H), 0.86 (s, 3H).

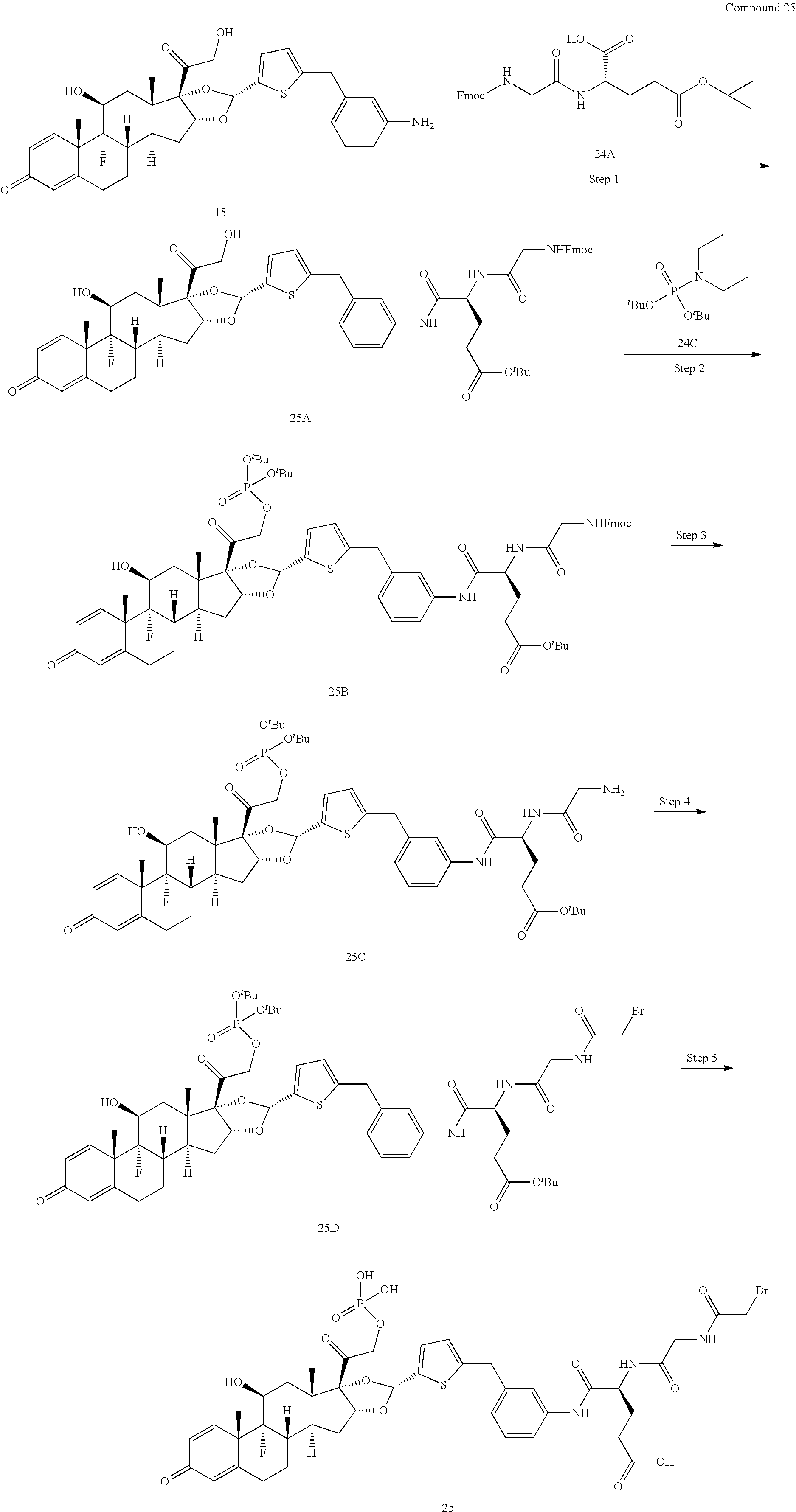

Step 1 Compound 24A (6.29 g, 13.0 mmol) was dissolved in pyridine (50.0 mL) and DMF (50.0 mL), EDCI (4.99 g, 26.0 mmol) was added to the reaction solution, and compound 16 (5.00 g, 8.68 mmol) was added to the reaction solution at 0° C. The resulting solution returned to 10° C. to 20° C. and stirred for 12 h. After the starting materials were completely consumed as detected by LCMS, the reaction solution was poured into water (150 mL), and the resulting mixture was extracted with ethyl acetate (150 m, 100 m, 50.0 mL). The organic phases were combined, washed with a saturated aqueous sodium chloride solution (30.0 mL), dried over anhydrous sodium sulfate, filtered and concentrated to give a yellow solid 25A (crude, 9.03 g, with negligible yield of crude product). MS-ES m/z 1058.4 [M+H]^{+.}

Step 2 Compound 25A (9.03 g, 8.68 mmol), tetrazole (4.40 g, 62.8 mmol) and compound 24C (13.0 g, 52.1 mmol) were dissolved in DMF (66.0 mL). The mixed solution was stirred at 20° C. for 2.5 h, and the internal temperature was reduced to 0° C. H₂O₂ (3.93 g, 34.7 mmol, 3.33 mL, 30% purity) was added to the reaction solution at 0° C., and the resulting solution returned to 10° C. to 20° C. and stirred for 1 h. After the starting materials were completely consumed as detected by LCMS, the mixture was poured into a saturated aqueous sodium sulfite solution (66.0 mL). The resulting mixture was diluted with water (132 mL) and extracted with ethyl acetate (200 mL, 100 mL). The organic phases were combined, washed with a saturated sodium chloride solution (50.0 mL), dried over anhydrous sodium sulfate, filtered and concentrated to give a yellow solid 25B (crude, 10.7 g, with negligible yield of crude product). MS-ESI: m/z 1250.5 [M+H]⁺.

Step 3 Compound 25B (10.7 g, 8.68 mmol) and piperidine (6.28 g, 73.8 mmol, 7.29 mL) were dissolved in acetonitrile (60.0 mL), and the mixed solution was stirred at 20° C. for 1 h. After the starting materials were completely consumed as detected by LCMS, the reaction solution was concentrated under reduced pressure to remove acetonitrile. The residue after concentration was slurried with petroleum ether (100 mL) for 2 h and filtered, and the filter cake was dried under vacuum to give a yellow solid 25C (8.77 g, yield: 83.8%). MS-ESI: m/z 1028.4 [M+H]^{+.}

Step 4 Bromoacetic acid (2.48 g, 17.8 mmol) and EEDQ (4.41 g, 17.8 mmol) were dissolved in DMF (63.0 mL), and the resulting solution was stirred at 20° C. for 1 h. Compound 25C (9.00 g, 8.91 mmol) was added to the reaction solution at 20° C., and the resulting solution was reacted at 20° C. for 2.5 h. After the starting materials were completely consumed as detected by LCMS, the reaction solution was diluted with dichloromethane (220 mL), washed twice with 1 N hydrobromic acid (160 mL×2), washed once with a saturated aqueous sodium bicarbonate solution (110 mL), washed once with a saturated aqueous sodium chloride solution (50.0 mL) and dried over anhydrous sodium sulfate. The organic phase was concentrated to dryness to give a yellow oily liquid 25D (crude, 10.1 g, with negligible yield of crude product).

MS-ESI: m/z 1148.4 [M+H]^{+.}

Step 5 Compound 25D (10.1 g, 8.91 mmol) was dissolved in dichloromethane (80.0 mL), and TFA (40.0 mL) was added to the reaction solution at 20° C. The resulting solution was stirred for 1 h. After there were 54.7% of the product as detected by LCMS, the reaction solution was concentrated under reduced pressure to give a crude product, which was purified by reversed-phase preparative chromatography [water (TFA)-acetonitrile] to give a yellow solid 25 (500 mg, yield: 8.58%, purity: 93.4%).

MS-ESI: m/z 980.0 [M+H]^{+.}

¹H NMR (400 MHz, DMSO-d₆) δ 11.66 (s, 2H), 9.93 (s, 1H), 8.51 (t, J=5.7 Hz, 1H), 8.24 (d, J=7.8 Hz, 1H), 7.50 (d, J=8.3 Hz, 1H), 7.46-7.40 (m, 1H), 7.30 (d, J=10.2 Hz, 1H), 7.20 (t, J=7.8 Hz, 1H), 7.07 (d, J=3.5 Hz, 1H), 6.93 (d, J=7.6 Hz, 1H), 6.73 (d, J=3.5 Hz, 1H), 6.25 (dd, J=10.1, 1.9 Hz, 1H), 6.06 (s, 1H), 5.83 (s, 1H), 5.51 (d, J=4.5 Hz, 1H), 4.96-4.84 (m, 2H), 4.57 (dd, J=18.2, 8.1 Hz, 1H), 4.44-4.35 (m, 1H), 4.23-4.16 (m, 1H), 4.07 (s, 2H), 3.94 (s, 2H), 3.88-3.73 (m, 2H), 2.70-2.54 (m, 2H), 2.40-2.32 (m, 1H), 2.32-2.22 (m, 2H), 2.22-2.09 (m, 1H), 2.06-1.91 (m, 2H), 1.90-1.76 (m, 3H), 1.71-1.57 (m, 3H), 1.49 (s, 4H), 1.44-1.29 (m, 1H), 0.87 (s, 3H).

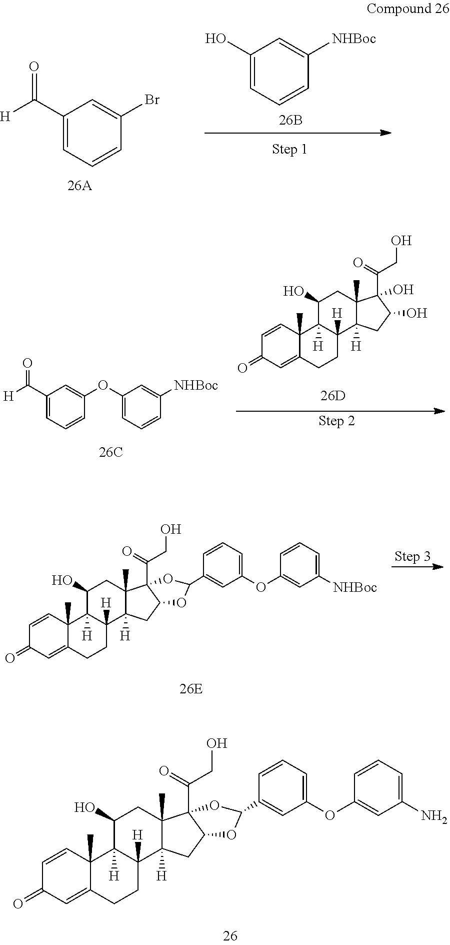

Step 1 Compound tert-butyl (3-hydroxyphenyl)carbamate 26B (3 g, 14.3 mmol, 1 eq) and compound 3-bromobenzaldehyde 26A (2.9 g, 15.7 mmol, 1.1 eq) were added to a three-necked flask and dissolved in dry DMF (50 mL). Cuprous iodide (0.27 g, 1.43 mmol, 0.1 eq) and L-proline (0.38 g, 1.43 mmol, 0.1 eq) were added, and the mixed solution was heated to 100° C. and reacted overnight under nitrogen atmosphere. After the reaction was completed as detected by TLC (PE:EA=20:1), 1 N hydrochloric acid was added to quench the reaction, and the reaction solution was extracted with dichloromethane, washed three times with 1 N hydrochloric acid, concentrated by rotary evaporation and purified by silica gel column chromatography (PE:EA=15:1) to give a white solid 26C (3.5 g, yield: 78.4%).

Step 2 Compound 26C (282 mg, 0.9 mmol), 26D (340 mg, 0.9 mmol) and MgSO₄ (600 mg) were added to a 25 mL three-necked flask, acetonitrile (5 mL) was added for dissolution, the mixed solution was stirred for 10 min, and then TfOH (407 mg, 2.7 mmol) was added. After 2 h, the reaction was completed as detected by TLC (PE/EA=1/1), the reaction solution was added to EA (40 mL), and the resulting solution was washed twice with water (50 mL×2), washed with a saturated NaCO₃ solution (50 mL) and saturated NaCl, and dried over Na₂SO₄ to give a light yellow solid 26E (447 mg, yield: 72%).

Step 3 Compound 26E (344 mg, 0.5 mmol) was dissolved in dry dichloromethane (10 mL), trifluoroacetic acid (1 mL) was added dropwise, and the mixed solution was cooled to 5° C. and stirred for 2 h. After the reaction was completed as detected by TLC (PE:EA=1:1), the reaction solution was diluted with DCM and concentrated by rotary evaporation to remove the solvent. The crude product was purified by preparative HPLC to give a white solid 26 (212 mg, yield: 36%). LC-MS 572.3 [M+H]^{+.}

¹H NMR (400 MHz, DMSO) δ 7.46-7.31 (m, 2H), 7.21 (d, J=7.6 Hz, 1H), 7.13-6.97 (m, 3H), 6.48 (d, J=7.7 Hz, 1H), 6.43-6.31 (m, 2H), 6.23 (dd, J=10.1, 1.7 Hz, 1H), 6.03 (s, 1H), 5.45 (s, 1H), 4.93 (d, J=4.8 Hz, 1H), 4.52 (d, J=19.5 Hz, 2H), 4.31-4.16 (m, 4H), 2.40-2.30 (m, 1H), 2.15-1.95 (m, 2H), 1.85-1.48 (m, 5H), 1.41 (s, 3H), 0.98-0.73 (m, 5H).

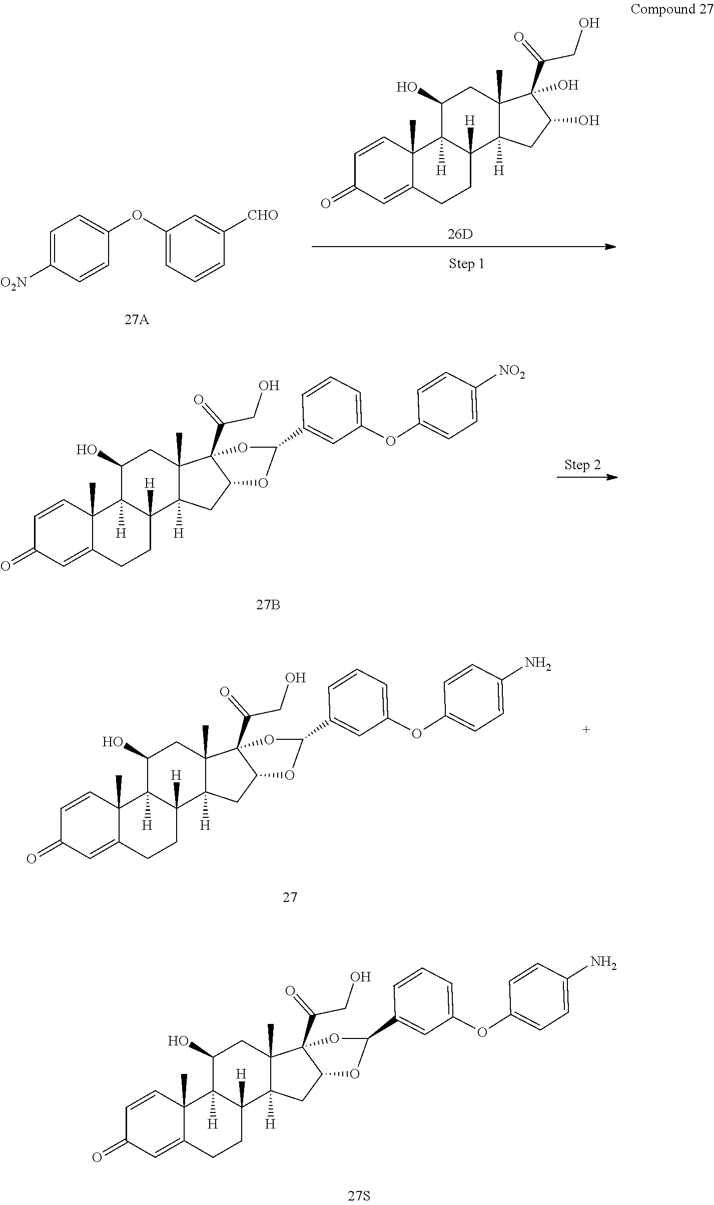

Step 1 27A (100 mg, 0.41 mmol), 26D (155 mg, 0.41 mmol) and magnesium sulfate (99 mg, 0.82 mmol) were added to anhydrous acetonitrile (3 mL), the mixed solution was cooled to 0° C., and trifluoromethanesulfonic acid (185 mg, 1.23 mmol) was slowly added dropwise. After addition, the resulting solution was stirred at 0° C. for 3 h. After the starting materials were completely consumed as detected by TLC (PE/EA=1/2), the reaction solution was filtered to remove magnesium sulfate, water (10 mL) and DCM (20 mL) were added to the filtrate, and liquid separation was performed. The organic phase was washed with saturated brine, dried over anhydrous sodium sulfate, filtered and concentrated to give a light yellow waxy solid 27B (110 mg, yield: 44%). MS-ESI: m/z 602.3 [M+H]^{+.}

Step 2 27B (100 mg, 0.17 mmol) and iron powder (93 mg, 1.66 mmol) were added to ethanol (3 mL). Ammonium chloride (89 mg, 1.66 mmol) was dissolved in water (1 mL), and the resulting solution was added dropwise to the system. Under nitrogen atmosphere, the system was heated to 60° C. and stirred for 3 h. After the starting materials were completely consumed as detected by LCMS, the reaction solution was filtered through celite, concentrated by rotary evaporation under reduced pressure and purified by preparative chromatography to give a white powder 27 (20 mg, yield: 21%) and compound 27S (10 mg, yield: 11%).

### Compound 27

MS-ESI: m/z 572.3 [M+H]^{+.}

¹H NMR (400 MHz, DMSO) δ7.51 (t, J=8.0 Hz, 1H), 7.33 (d, J=10.4 Hz, 1H), 7.20-7.30 (m, 1H), 7.00-7.15 (m, 6H), 6.19 (dd, J=10, 2.0 Hz, 1H), 5.99 (s, 1H), 5.48 (s, 1H), 4.90-5.00 (m, 1H), 4.80-4.90 (m, 1H), 4.53 (d, J=19.2 Hz, 1H), 4.31 (s, 1H), 4.25 (d, J=19.2 Hz, 1H), 2.30-2.40 (m, 1H), 1.90-2.20 (m, 2H), 1.60-1.80 (m, 5H), 1.43 (s, 3H), 0.85-1.05 (m, 5H).

### Compound 27S

MS-ESI: m/z 572.3 [M+H]^{+.}

¹H NMR (400 MHz, DMSO) δ 7.40 (t, J=7.9 Hz, 1H), 7.33 (d, J=10.1 Hz, 1H), 7.13 (d, J=7.3 Hz, 2H), 7.03 (dd, J=19.8, 8.0 Hz, 4H), 6.87 (s, 1H), 6.19 (dd, J=10.1, 1.8 Hz, 1H), 6.12 (s, 1H), 5.96 (s, 1H), 5.28 (d, J=6.6 Hz, 1H), 4.90-4.75 (m, 1H), 4.27-4.35 (m, 1H), 4.21 (d, J=19.2 Hz, 1H), 4.03 (d, J=19.2 Hz, 1H), 2.62-2.55 (m, 1H), 2.38-2.27 (m, 1H), 2.15-1.95 (m, 2H), 1.91-1.66 (m, 5H), 1.41 (s, 3H), 1.28-1.10 (m, 1H), 1.10-1.00 (m, 1H), 0.89 (s, 3H).

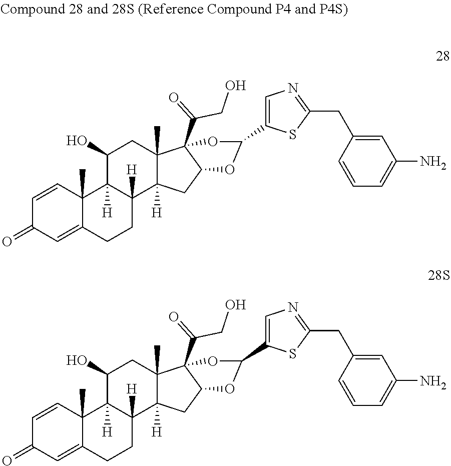

It was synthesized according to the reference patent WO2021216913A1.

### ADC Preparation Schemes and Examples

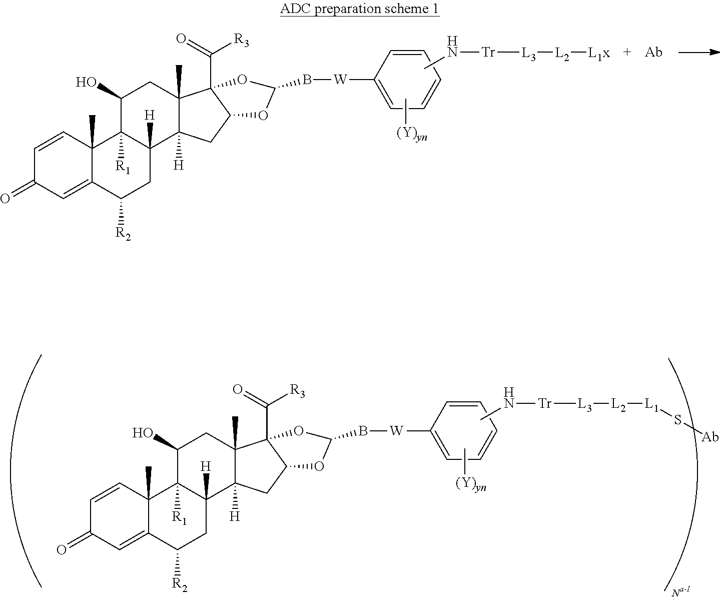

The ligand Ab could be reacted with a compound of any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least one free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C), formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab;
Y, yn, R₁, R₂, R₃, B, W, Tr, L₁, L₂ and L₃ may be defined as Y, yn, R₁, R₂, R₃, B, W, Tr, L₁, L₂ and L₃ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; L₁ may represent the structure after coupling with L₁ₓ coupled with sulfhydryl;
L₁ₓ may comprise a group selected from the group consisting of the following:

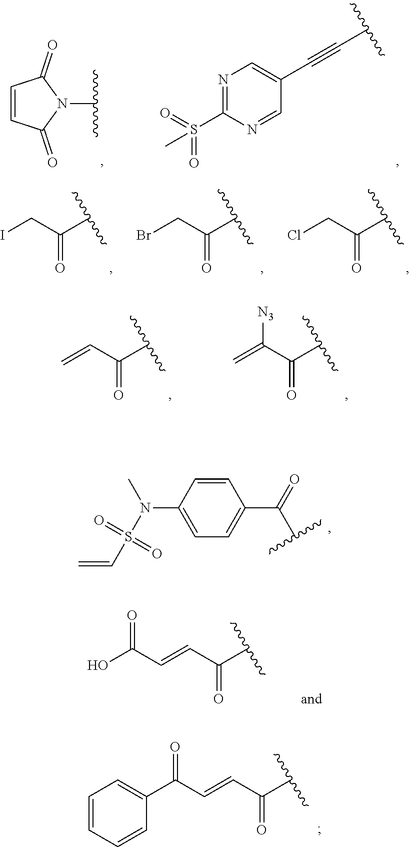

the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above.

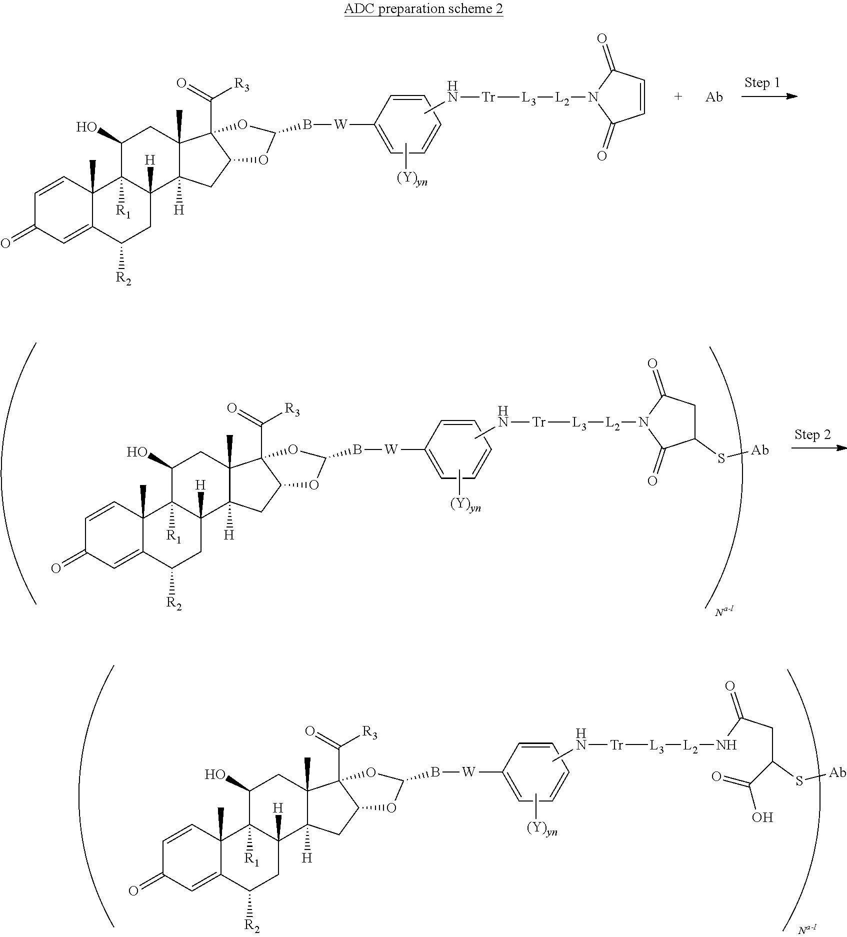

Step 1: the ligand Ab could be reacted with a compound of formula (I-D), formula (I-a1-D), formula (I-a2-D), formula (I-a4-C), formula (I-a4-D), formula (II-D), formula (II-a1-D), formula (II-a2-D), formula (II-a4-C), formula (II-a4-D), formula (II-b1-D), formula (II-b2-D), formula (II-b3-D) or formula (II-b4-D) of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least one free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab; Step 2: the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application was incubated in an alkaline buffer at a selected temperature for a selected time to obtain another compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application;
Y, yn, R₁, R₂, R₃, B, W, Tr, L₂ and L₃ may be defined as Y, yn, R₁, R₂, R₃, B, W, Tr, L₂ and L₃ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively;
the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above; the selected temperature may be 5° C. to 60° C.; the incubation time may be 0 h to 72 h.

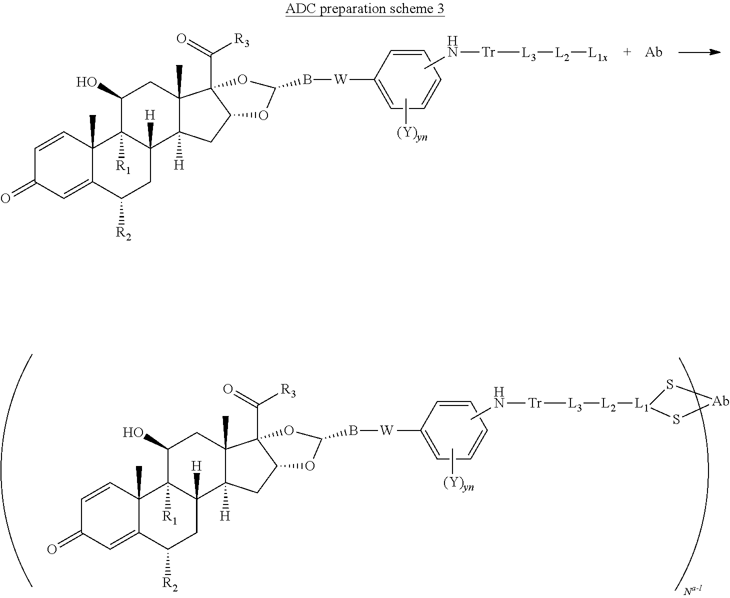

The ligand Ab was reacted with a compound of any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) having a group capable of being coupled with two sulfhydryl of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least two free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab;
Y, yn, R₁, R₂, R₃, B, W, Tr, L₁, L₂ and L₃ may be defined as Y, yn, R₁, R₂, R₃, B, W, Tr, L₁, L₂ and L³ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; L₁ represents the structure after coupling with L₁ₓ coupled with two sulfhydryl;
the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) having a group capable of being coupled with two sulfhydryl may comprise an L₁ group selected from the group consisting of the following:

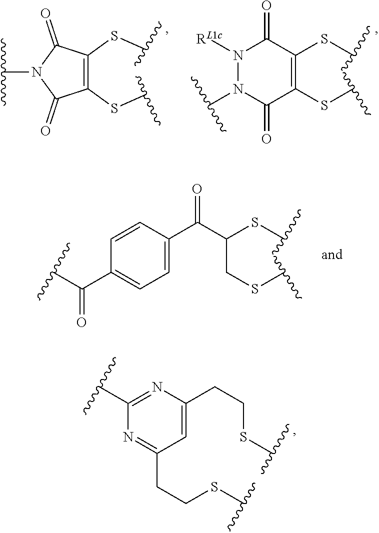

wherein each of R^{L1a}, R^{L1b} and R^{L1C} is independently selected from the group consisting of the following: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H, alkyl, alkenyl, alkynyl, alcyl, heterocyclyl, aryl and heteroaryl;
the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above.

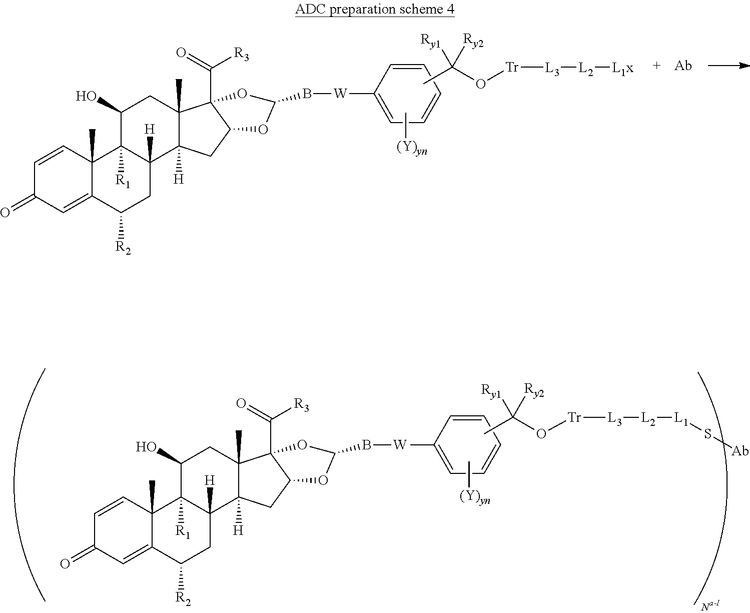

The ligand Ab could be reacted with a compound of any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least one free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C), formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab;
Y, yn, R₁, R₂, R₃, B, W, R_{y1}, R_{y2}, Tr, L₁, L₂ and L₃ may be defined as Y, yn, R₁, R₂, R₃, B, W, R_{y1}, R_{y2}, Tr, L₁, L₂ and L₃ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; L₁ may represent the structure after coupling with L₁ₓ coupled with sulfhydryl;
L₁ₓ may comprise a group selected from the group consisting of the following:

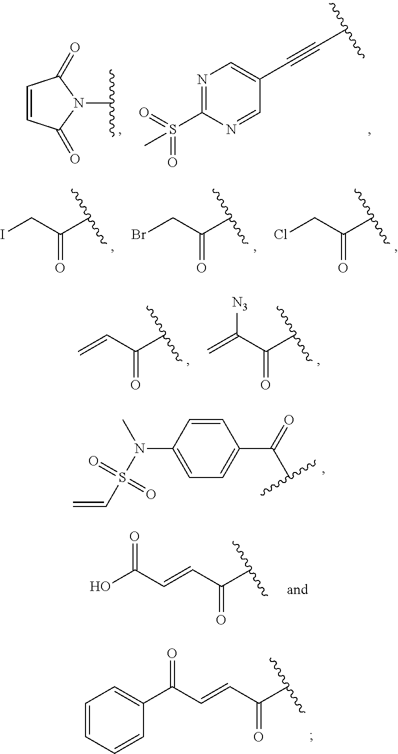

the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above.

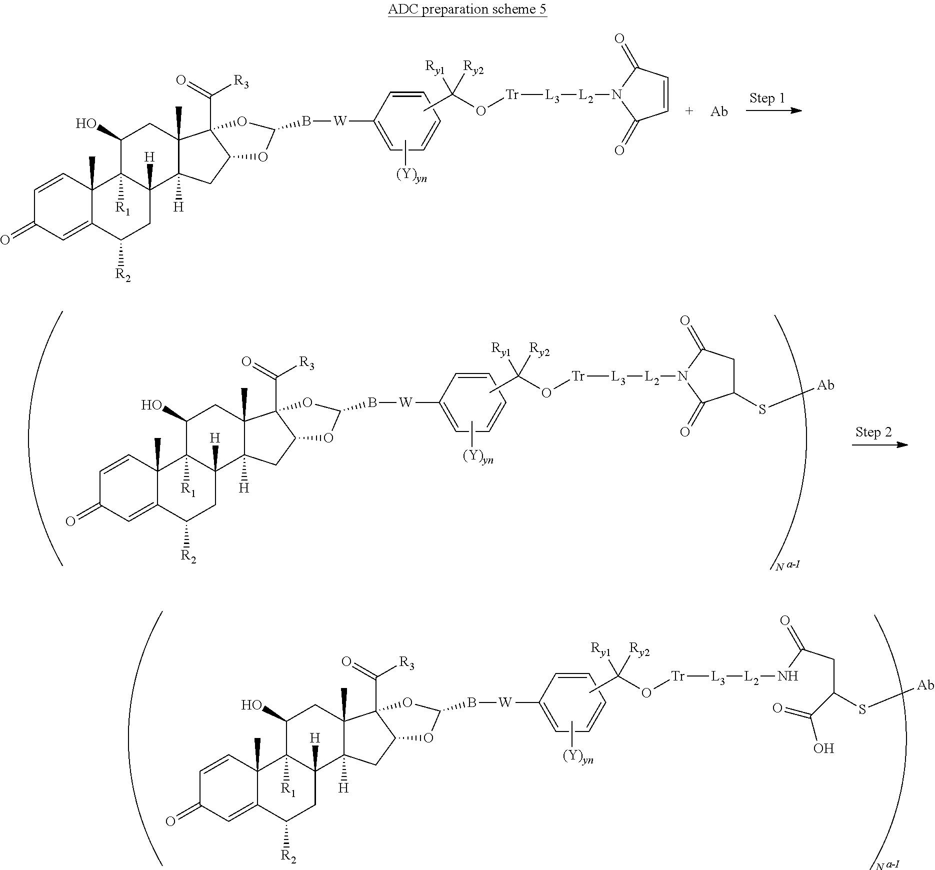

Step 1: the ligand Ab could be reacted with a compound of formula (I-D), formula (I-a1-D), formula (I-a2-D), formula (I-a-3D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least one free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab;

Step 2: the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application was incubated in an alkaline buffer at a selected temperature for a selected time to obtain another compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application;
Y, yn, R₁, R₂, R₃, B, W, R_{y1}, R_{y2}, Tr, L₂ and L₃ may be defined as Y, yn, R₁, R₂, R₃, B, W, Ry₁, R_{y2}, Tr, L₂ and L₃ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively;
the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above; the selected temperature may be 5° C. to 60° C.; the incubation time may be 0 h to 72 h.

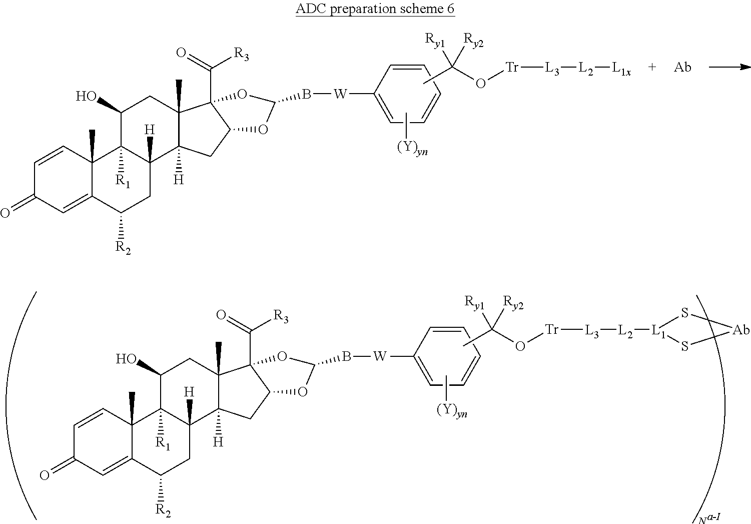

The ligand Ab was reacted with a compound of any one of formula (I-D), formula (I-a1-D), formula (I-a2-D), formula (I-a3-D), formula (I-a4-D), formula (II-D), formula (II-a1-D), formula (II-a2-D), formula (II-a3-D), formula (II-a4-D), formula (II-b1-D), formula (II-b2-D), formula (II-b3-D) or formula (II-b4-D) having a group capable of being coupled with two sulfhydryl of the present application in an acidic, neutral or alkaline buffer to obtain a compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C);
wherein Ab is a ligand containing at least two free sulfhydryl (—SH), wherein the free sulfhydryl may be obtained by reducing the ligand by using a reducing agent; the reducing agent includes, but is not limited to, tris(2-carboxyethyl)phosphine, mercaptoethanol, dithiothreitol, cysteine, reduced glutathione, and the like; particularly, disulfide bonds (—S—S—) between ligand chains may be reduced to form free sulfhydryl; wherein the S atom in the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) may be derived from sulfhydryl in Ab;
Y, yn, R₁, R₂, R₃, B, W, R_{y1}, R_{y2}, Tr, L₁, L₂ and L₃ may be defined as Y, yn, R₁, R₂, R₃, B, W, R_{y1}, R_{y2}, Tr, L₁, L₂ and L₃ in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; Ab and N^{a-1} may be defined as Ab and N^{a-1} in the compound of any one of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) of the present application, respectively; L₁ represents the structure after coupling with L₁ₓ coupled with two sulfhydryl;
the compound of formula (I-C), formula (I-a1-C), formula (I-a2-C), formula (I-a3-C), formula (I-a4-C), formula (II-C), formula (II-a1-C), formula (II-a2-C), formula (II-a3-C), formula (II-a4-C), formula (II-b1-C), formula (II-b2-C), formula (II-b3-C) or formula (II-b4-C) having a group capable of being coupled with two sulfhydryl may comprise an L₁ group selected from the group consisting of the following:

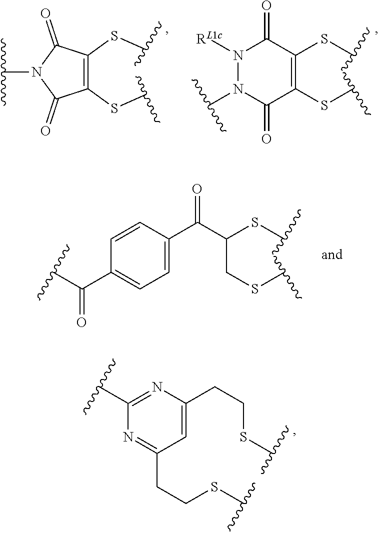

wherein each of R^{L1a}, R^{L1b} and R^{L1c} is independently selected from the group consisting of the following: hydrogen, protium, deuterium, tritium, halogen, —NO₂, —CN, —OH, —SH, —NH₂, —C(O)H, —CO₂H, —C(O)C(O)H, —C(O)CH₂C(O)H, —S(O)H, —S(O)₂H, —C(O)NH₂, —SO₂NH₂, —OC(O)H, —N(H)SO₂H, alkyl, alkenyl, alkynyl, alcyl, heterocyclyl, aryl and heteroaryl;
the buffer is selected from the following buffers with a pH of 2 to 12: citric acid-sodium citrate buffer, phosphoric acid-sodium phosphate buffer, phosphoric acid-potassium phosphate buffer, sodium dihydrogen phosphate-disodium hydrogen phosphate buffer, potassium dihydrogen phosphate-dipotassium hydrogen phosphate buffer, succinic acid-sodium succinate buffer, acetic acid-sodium acetate buffer, boric acid-borax buffer, boric acid-potassium borate buffer, borax-sodium hydroxide buffer, histidine-hydrochloric acid buffer, glycine-sodium hydroxide buffer, arginine-hydrochloric acid buffer, sodium bicarbonate-sodium carbonate buffer, potassium bicarbonate-potassium carbonate buffer, Tris-hydrochloric acid buffer, aqueous ammonia-ammonium chloride buffer, barbiturate sodium-hydrochloric acid buffer, borax-sodium carbonate buffer, boric acid-potassium chloride buffer, and a combination of two or more of the above.

### Example 2

In the present application, a 20 mM aqueous histidine buffer (pH=5.5) was obtained by adjusting the pH of a 20 mM aqueous histidine solution to 5.5 with HOAc. Stock solution 1: 10 mM EDTA buffer, pH=4.7; stock solution 2: 800 mM aqueous sodium citrate buffer, pH=8.4; stock solution 3: 500 mM aqueous urea solution; stock solution 4: 10 mM aqueous tris(2-carboxyethyl)phosphine solution.

The protein purity of the ADC in the present application was detected by SEC method, and the parameters are shown below.

TABLE 1

| Instrument | Agilent Technologies 1260 | |
|---|---|---|
| Chromatography | TOSON-G3000SWXL, 300*7.8 mm, 5 μm | |
| column | | |
| Column | 22° | C. |
| temperature | | |
| Wavelength | 280 | nm |
| Injection | 30~50 | μg |
| volume | | |
| Mobile phase | 0.2M K2HPO4/KH2PO4, 0.25M KCl, | |
| | 15% (v/v) 2-propanol, pH 7.0 | |
| Gradient | Time (min) | Flow rate (mL/min) |
| | 0.0 | 0.75 |
| | 18.0 | 0.75 |

The DAR value of the ADC in the present application can be detected by LC-MS method. The experimental procedure was as follows: to 40.0 μg of ADC sample solution were added 75.0 μL 8.0 mol/L guanidine hydrochloride (Gdn-HCl), 5.0 μL 1.0 mol/L Tris-HCl, and 2.0 μL 1 mol/L DTT. The solution was diluted to 100.0 μL with ultrapure water, mixed well and incubated at 22° C. for 30 min. Drug/antibody ratio (DAR) was then analyzed using LC-MS. The LC parameters are shown below.

TABLE 2

| Instrument | Agilent Technologies 1260 | |
|---|---|---|
| Reversed-phase | Agilent, PLRP-S 2.1*50 mm, 8 μm | |
| chromatography | | |
| column | | |
| Column | 80° | C. |
| temperature | | |
| Sample | 2~8° | C. |
| temperature | | |
| Detection | 280 | nm |
| wavelength | | |
| Injection | 10.0 | μL |

| volume | | | | |
|---|---|---|---|---|
| Mobile phase | Mobile phase A: 0.025% TFA and 0.1% FA in | | | |
| | ultrapure water | | | |
| | Mobile phase B: 0.025% TFA and 0.1% FA in | | | |
| | ACN | | | |
| | Time | | | Flow rate |
| Gradient | (min) | % A | % B | (mL/min) |
| | 0.0 | 75 | 25 | 0.5 |
| | 0.0 | 75 | 25 | |
| | 0.7 | 66 | 34 | |
| | 5.0 | 55 | 45 | |
| | 6.0 | 10 | 90 | |
| | 7.0 | 10 | 90 | |
| | 7.1 | 75 | 25 | |
| | 10.0 | 75 | 25 | |

The MS parameters are shown below.

TABLE 3

| | Agilent Technologies 6224 TOF | |
|---|---|---|
| Instrument | LC/MS | |
| Ionization mode | Positive | |
| Gas temperature | 350° | C. |
| Air flow | 13 | L/min |
| Atomizer pressure | 45 | psig |
| Capillary voltage | 5000 | V |
| Crushing voltage | 250 | V |
| Voltage of taper hole | 65 | V |
| Peak-to-peak value of | 750 | V |
| radio frequency voltage on | | |
| octupole | | |
| Acquisition mode | MS (seg) | |
| Mass range | 500-8000 | m/z |
| Acquisition rate | 1 | spectra/s |
| Acquisition time | 1.4-7.0 | min |

The DAR value of the ADC in the present application can also be detected by HIC method, and the parameters are shown below.

TABLE 4

| Chromatography | | | |
|---|---|---|---|
| column | TSKgel Butyl-NPR, 4.6 mm *3.5 cm, 2.5 μm | | |
| Column | 25° C. | | |
| temperature | | | |
| Detection | 280 nm | | |
| wavelength | | | |
| Sample load | 8 μL | | |
| Mobile phase | Mobile phase A: 1.5M (NH₄)₂SO₄, | | |
| | 0.05M K₂HPO₄•3H₂O (pH = 7.0) | | |
| | Mobile phase B: 21.3 mM KH₂PO₄, | | |
| | 28.6 mM K₂HPO₄•3H₂O, 25% | | |
| | (V/V) IPA (pH = 7.0) | | |
| Gradient | Time (min) | % B | Flow rate (mL/min) |
| | 0.0 | 0.0 | 0.6 |
| | 2.0 | 0.0 | |
| | 15.0 | 100.0 | |
| | 16.0 | 100.0 | |
| | 17.0 | 0.0 | |
| | 20.0 | 0.0 | |

Murine anti-TNFα mIgG2a 8c11: the light chain amino acid sequence is set forth in SEQ ID NO: 31 and the heavy chain amino acid sequence is set forth in SEQ ID NO: 32, prepared with reference to WO2015191783A2.

Antibody adalimumab (Humira): the light chain amino acid sequence is set forth in SEQ ID NO: 9 and the heavy chain amino acid sequence is set forth in SEQ ID NO: 10, prepared with reference to U.S. Pat. No. 6,090,382A.

Antibody BIIB059: the light chain amino acid sequence is set forth in SEQ ID NO: 42 and the heavy chain amino acid sequence is set forth in SEQ ID NO: 37, prepared with reference to CN105452295B.

Antibody iscalimab: the light chain amino acid sequence is set forth in SEQ ID NO: 19 and the heavy chain amino acid sequence is set forth in SEQ ID NO: 20, prepared with reference to CN105949314B.

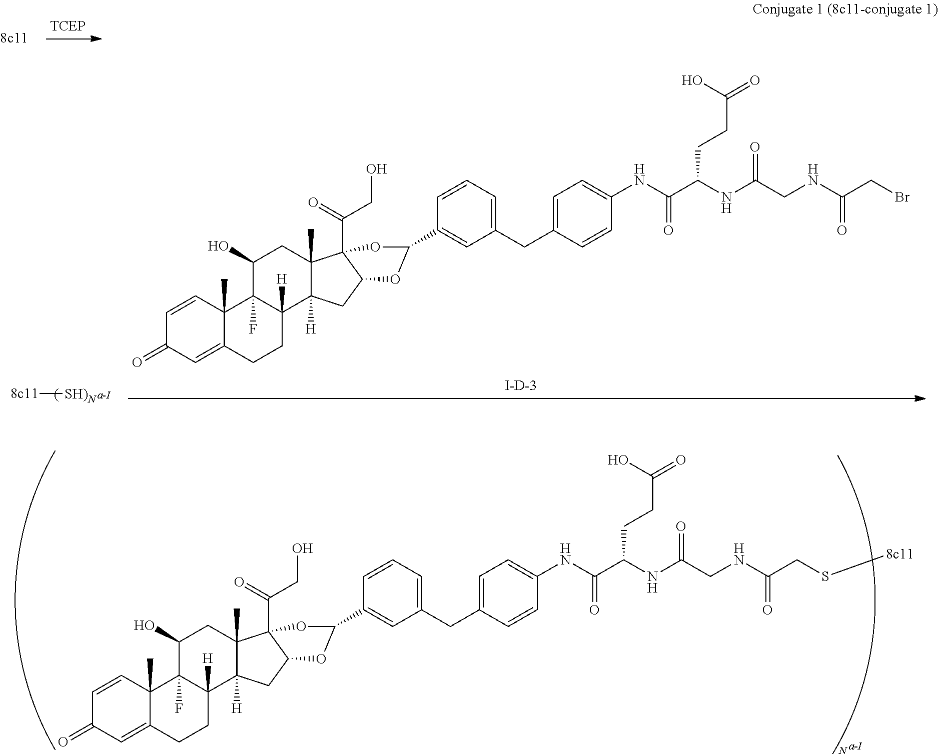

To an aqueous PBS buffer (0.05 M aqueous PBS buffer at pH=6.5; 7.5 mL, 9.96 mg/mL, 0.505 nmol) of antibody 8c11 was added the prepared aqueous solution of tris(2-carboxyethyl)phosphine (10 mM, 0.162 mL, 1.616 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 12.0 mL of the solution was taken out and used in the next step.

Compound I-D-3 (7.26 mg, 8.11 nmol) was dissolved in 0.10 mL of DMA, the resulting solution was added to the above solution (12.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate 1 (2.313 mg/mL, 23.465 mL).

N^{a-1} was found to be 4.20 as detected by Reverse Phase.

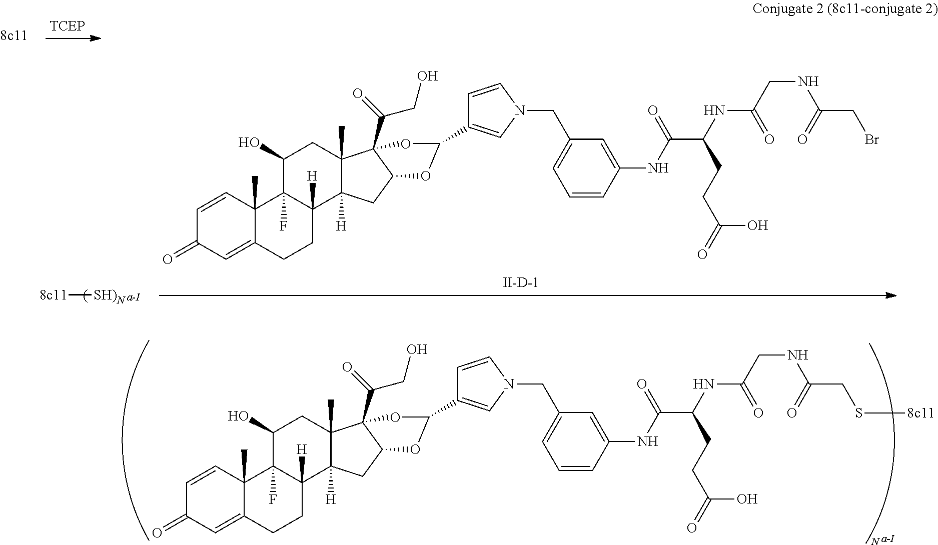

To an aqueous PBS buffer (0.05 M aqueous PBS buffer at pH=6.5; 2.5 mL, 9.96 mg/mL, 0.168 nmol) of antibody 8c11 was added the prepared aqueous solution of tris(2-carboxyethyl)phosphine (10 mM, 0.054 mL, 0.54 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 2.0 mL of the solution was taken out and used in the next step.

Compound II-D-1 (1.67 mg, 2.02 nmol) was dissolved in 0.10 mL of DMA, the resulting solution was added to the above solution (2.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate 2 (1.8 mg/mL, 4.1 mL).

N^{a-1} was found to be 2.58 as detected by LC-MS.

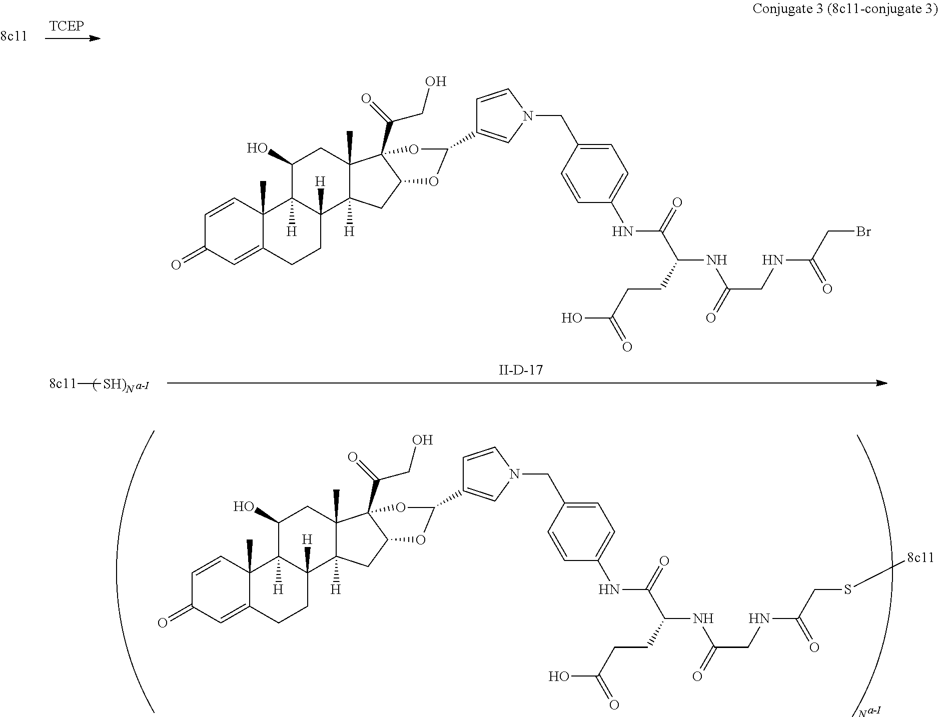

To an aqueous PBS buffer (0.05 M aqueous PBS buffer at pH=6.5; 2.5 mL, 9.96 mg/mL, 0.168 nmol) of antibody 8c11 was added the prepared aqueous solution of tris(2-carboxyethyl)phosphine (10 mM, 0.054 mL, 0.54 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 2.0 mL of the solution was taken out and used in the next step.

Compound II-D-17 (1.67 mg, 2.02 nmol) was dissolved in 0.10 mL of DMA, the resulting solution was added to the above solution (2.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate 6 (1.5 mg/mL, 4.2 mL).

N^{a-1} was found to be 3.22 as detected by LC-MS.

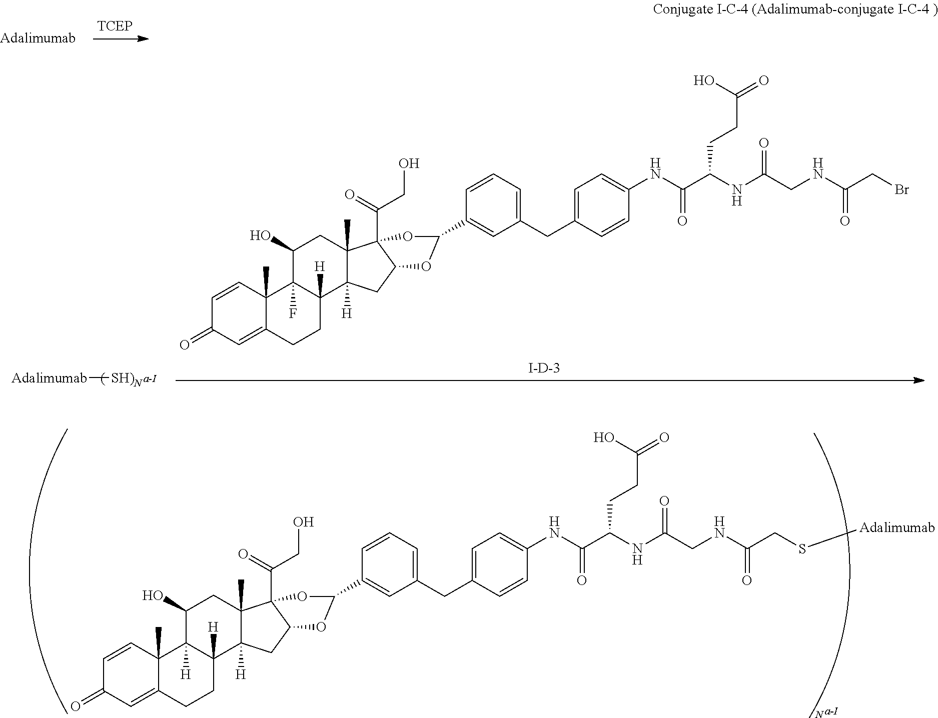

To an aqueous PBS buffer (0.05 M aqueous PBS buffer at pH=6.5; 2.5 mL, 9.96 mg/mL, 0.168 nmol) of antibody adalimumab was added the prepared aqueous solution of tris(2-carboxyethyl)phosphine (10 mM, 0.054 mL, 0.54 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 2.0 mL of the solution was taken out and used in the next step.

Compound I-D-3 (1.67 mg, 2.02 nmol) was dissolved in 0.10 mL of DMA, the resulting solution was added to the above solution (2.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate I-C-4 (1.6 mg/mL, 4.6 mL).

N^{a-1} could be detected by LC-MS.

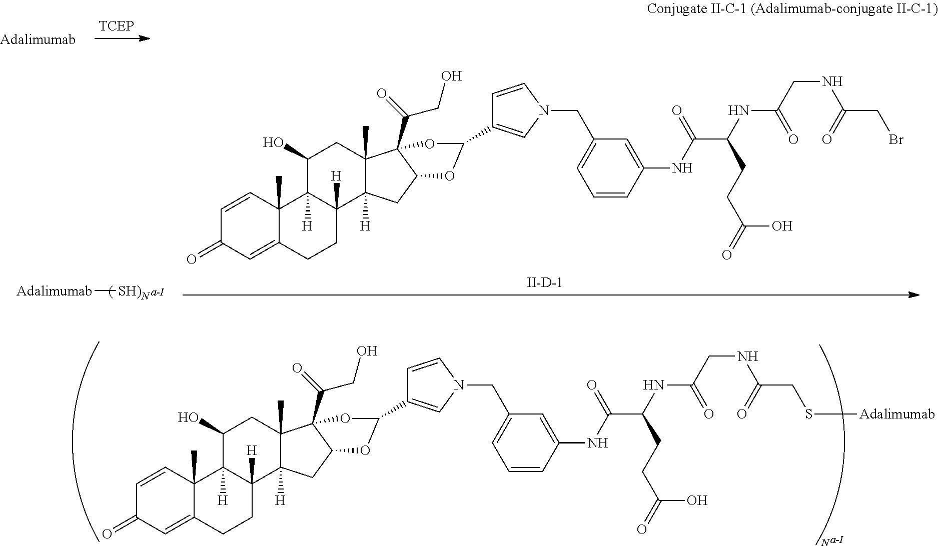

To an aqueous PBS buffer (0.05 M aqueous PBS buffer at pH=6.5; 2.5 mL, 9.96 mg/mL, 0.168 nmol) of antibody adalimumab was added the prepared aqueous solution of tris(2-carboxyethyl)phosphine (10 mM, 0.054 mL, 0.54 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 2.0 mL of the solution was taken out and used in the next step.

Compound II-D-1 (1.67 mg, 2.02 nmol) was dissolved in 0.10 mL of DMA, the resulting solution was added to the above solution (2.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate II-C-1 (1.7 mg/mL, 3.2 mL). N^{a-1} could be detected by LC-MS.

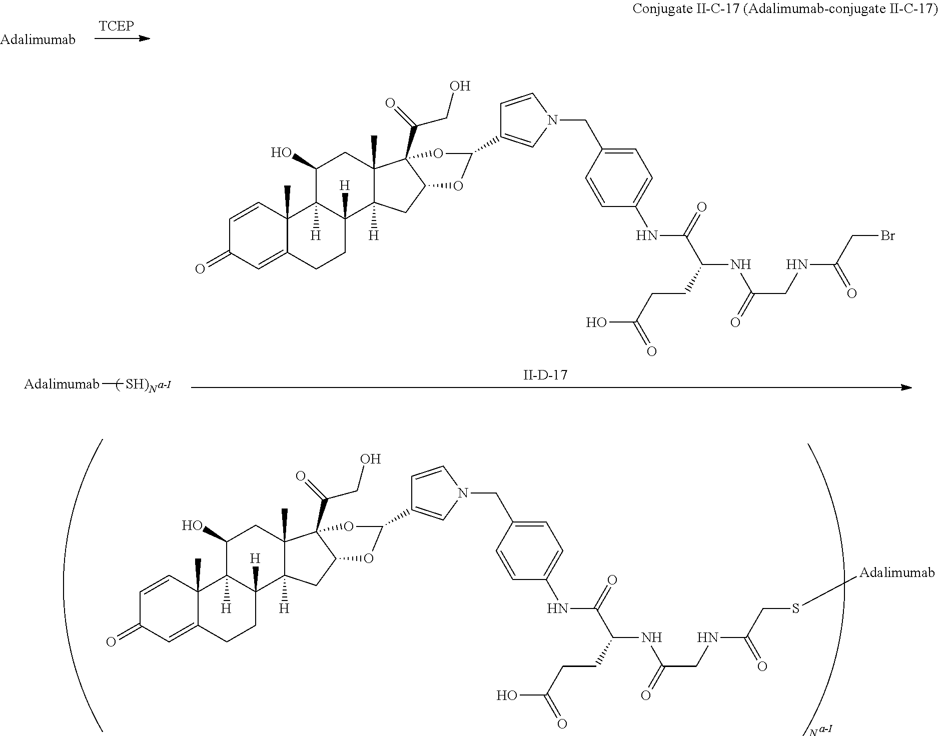

To an aqueous PBS buffer (0.05 M aqueous PBS buffer at pH=6.5; 2.5 mL, 9.96 mg/mL, 0.168 nmol) of antibody adalimumab was added the prepared aqueous solution of tris(2-carboxyethyl)phosphine (10 mM, 0.054 mL, 0.54 nmol) at 37° C., and the reaction solution was placed in a water bath shaker, shaken and reacted at 37° C. for 3 h before the reaction was stopped; the reaction solution was cooled to 25° C. in a water bath, diluted to 5.0 mg/mL, and 2.0 mL of the solution was taken out and used in the next step.

Compound II-D-17 (1.67 mg, 2.02 nmol) was dissolved in 0.10 mL of DMA, the resulting solution was added to the above solution (2.0 mL), and the mixed solution was placed in the water bath shaker, shaken and reacted at 25° C. for 3 h before the reaction was stopped. The reaction solution was desalted and purified through Sephadex G25 gel column (eluent: 0.05 M aqueous histidine buffer at pH=5.5, containing 0.001 M EDTA) to give conjugate II-C-17 (1.6 mg/mL, 3.1 mL). N^{a-1} could be detected by LC-MS.

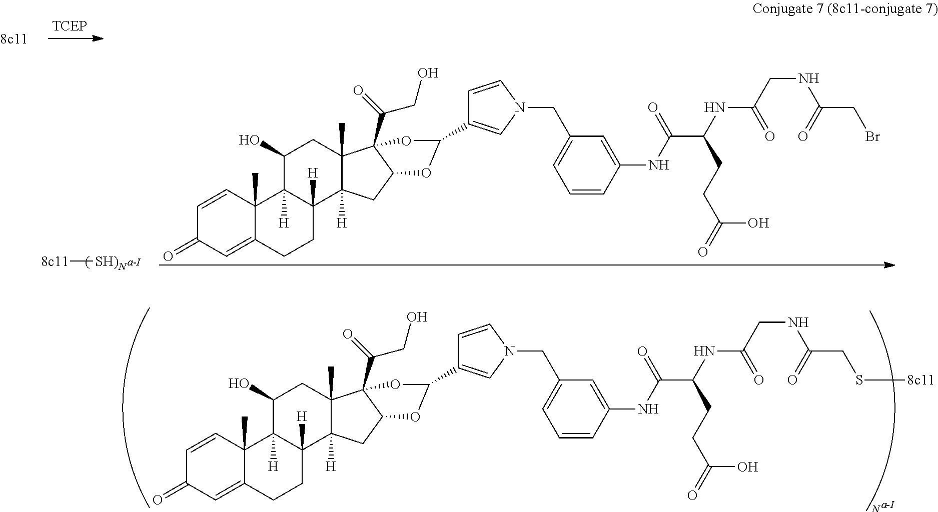

To an aqueous NaOAc buffer (0.05 M aqueous NaOAc buffer at pH=5.5; 1.14 mL, 10.93 mg/mL, 0.084 μmol) of antibody 8c11 was added the prepared aqueous solution of tris(2-carboxyethyl)phosphine (10 mM, 0.042 mL, 0.420 μmol) at 22° C., and the reaction solution was placed in a water bath shaker, shaken and reacted at 22° C. for 2 h before the reaction was stopped. Stock solution 1 and stock solution 2 were added such that the buffer system contained 0.29 mM EDTA and 11.53 mM sodium citrate.

Compound 12 (1.09 mg, 1.26 μmol) was dissolved in 0.11 mL of DMSO, the resulting solution was added to the above solution, and the mixed solution was placed in the water bath shaker, shaken and reacted at 4° C. for 1.3 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 7 (10 mg, 2.4 mg/mL, yield: 80%). N^{a-1} was found to be 3.72 as detected by RP.

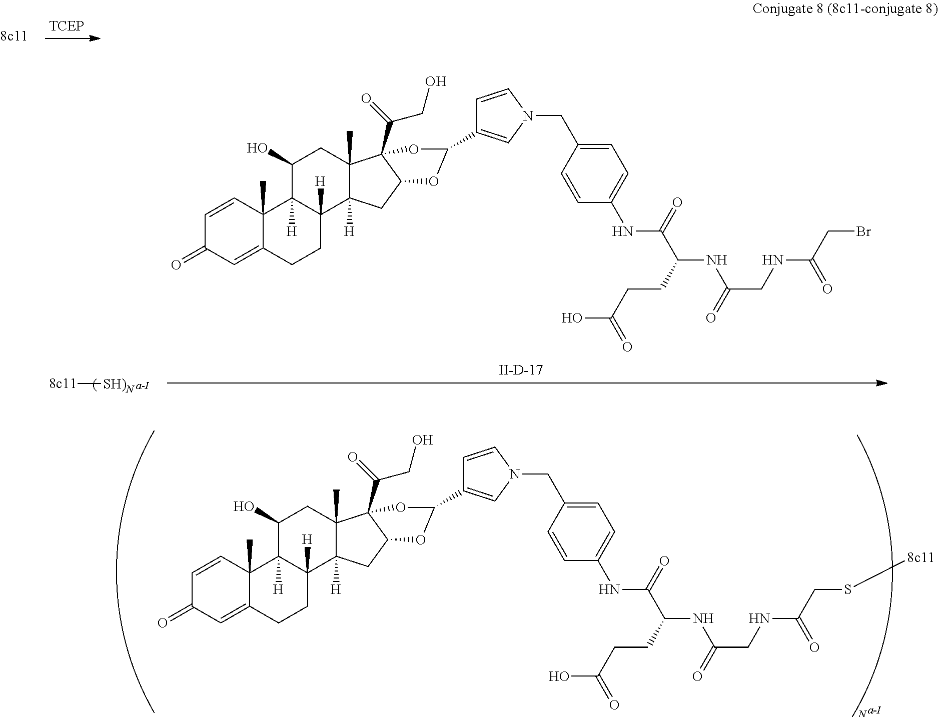

To an aqueous NaOAc buffer (0.05 M aqueous NaOAc buffer at pH=5.5; 1.14 mL, 10.93 mg/mL, 0.084 μmol) of antibody 8c11 was added the prepared aqueous solution of tris(2-carboxyethyl)phosphine (10 mM, 0.042 mL, 0.420 μmol) at 22° C., and the reaction solution was placed in a water bath shaker, shaken and reacted at 22° C. for 2 h before the reaction was stopped. Stock solution 1 and stock solution 2 were added such that the buffer system contained 0.29 mM EDTA and 11.53 mM sodium citrate.

Compound 13 (1.09 mg, 1.26 μmol) was dissolved in 0.11 mL of DMSO, the resulting solution was added to the above solution, and the mixed solution was placed in the water bath shaker, shaken and reacted at 4° C. for 1.3 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 8 (10 mg, 2.51 mg/mL, yield: 80%). N^{a-1} was found to be 1.84 as detected by RP.

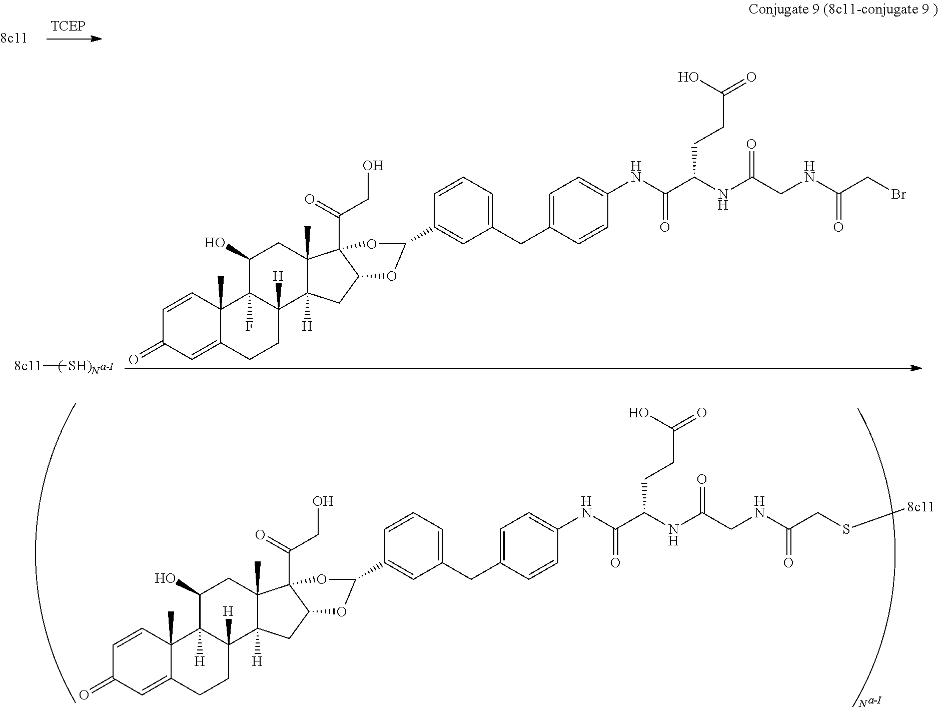

To an aqueous NaOAc buffer (0.05 M aqueous NaOAc buffer at pH=5.5; 18.24 mL, 3.4 mg/mL, 0.419 μmol) of antibody 8c11 was added the prepared aqueous solution of tris(2-carboxyethyl)phosphine (10 mM, 0.149 mL, 1.49 μmol) at 22° C., and the reaction solution was placed in a water bath shaker, shaken and reacted at 22° C. for 3 h before the reaction was stopped. Stock solution 1 and stock solution 2 were added such that the buffer system contained 0.29 mM EDTA and 11.53 mM sodium citrate.

Compound 14 (6.75 mg, 7.52 μmol) was dissolved in 0.68 mL of DMSO, the resulting solution was added to the above solution, and the mixed solution was placed in the water bath shaker, shaken and reacted at 22° C. for 9 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 9 (62 mg, 2.31 mg/mL, yield: 1000%). N^{a-1} was found to be 4.2 as detected by RP.

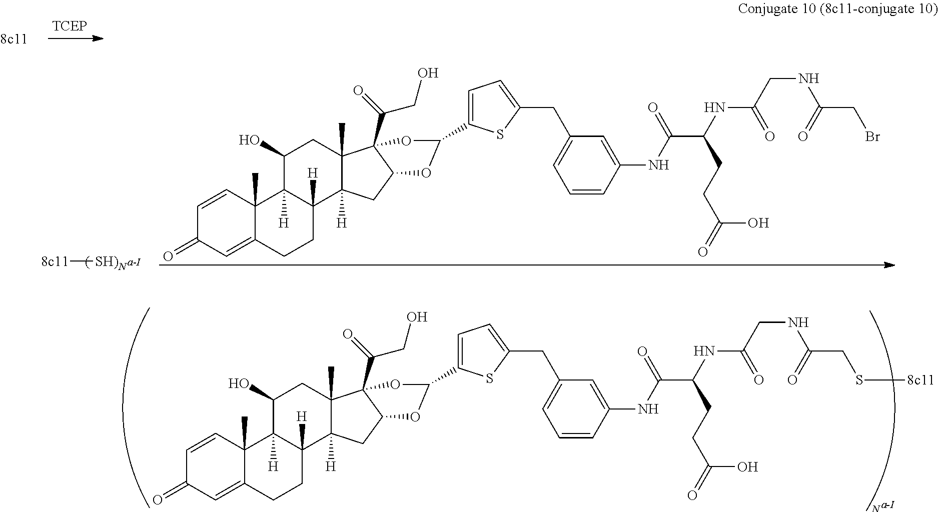

To an aqueous NaOAc buffer (0.05 M aqueous NaOAc buffer at pH=7.4, containing 0.29 mM EDTA and 11.53 mM sodium citrate; 2.94 mL, 3.4 mg/mL, 0.068 μmol) of antibody 8c11 were added the prepared aqueous solution of tris(2-carboxyethyl)phosphine (10 mM, 0.031 mL, 0.31 μmol) and a solution of compound 21 (0.9 mg, 1.02 μmol) in DMSO (0.09 mL) at 22° C., and the reaction solution was placed in a water bath shaker, shaken and reacted at 22° C. for 3 h before the reaction was stopped.

To an aqueous NaOAc buffer (0.05 M aqueous NaOAc buffer at pH=7.4, containing 0.29 mM EDTA and 11.53 mM sodium citrate; 7.35 mL, 3.4 mg/mL, 0.169 μmol) of antibody 8c 11 were added the prepared aqueous solution of tris(2-carboxyethyl)phosphine (10 mM, 0.076 mL, 0.76 μmol) and a solution of compound 21 (2.2 mg, 2.54 μmol) in DMSO (0.22 mL) at 22° C., and the reaction solution was placed in the water bath shaker, shaken and reacted at 22° C. for 3 h before the reaction was stopped.

The reaction solutions were combined. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 10 (33.1 mg, 2.41 mg/mL, yield: 94%).
N^{a-1} was found to be 4.79 as detected by RP.

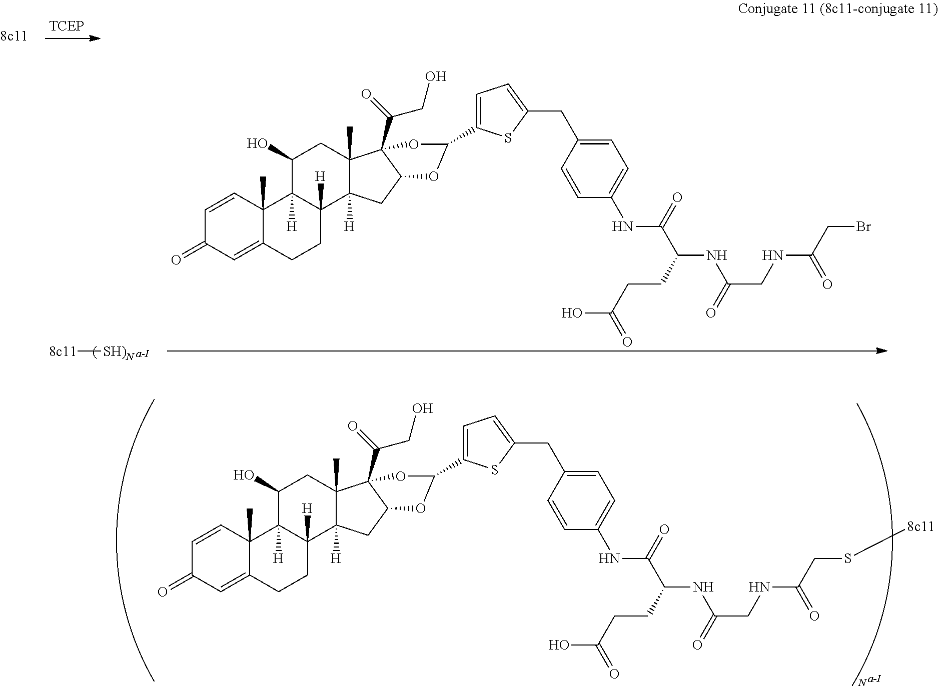

To an aqueous NaOAc buffer (0.05 M aqueous NaOAc buffer at pH=7.4, containing 0.29 mM EDTA and 11.53 mM sodium citrate; 10.3 mL, 3.4 mg/mL, 0.236 μmol) of antibody 8c11 were added the prepared aqueous solution of tris(2-carboxyethyl)phosphine (10 mM, 0.10 mL, 1.04 μmol) and a solution of compound 22 (3.1 mg, 3.540 μmol) in DMSO (0.31 mL) at 22° C., and the reaction solution was placed in the water bath shaker, shaken and reacted at 22° C. for 3 h before the reaction was stopped.

⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 11 (32.6 mg, 2.38 mg/mL, yield: 93%).
N^{a-1} was found to be 4.93 as detected by RP.

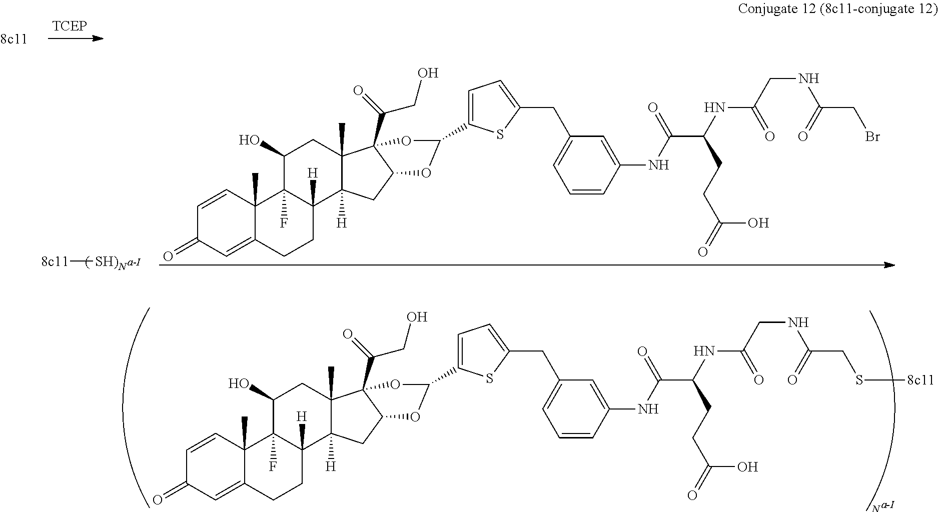

To an aqueous NaOAc buffer (0.05 M aqueous NaOAc buffer at pH=7.4, containing 0.29 mM EDTA and 11.53 mM sodium citrate; 6.47 mL, 3.4 mg/mL, 0.149 μmol) of antibody 8c11 were added the prepared aqueous solution of tris(2-carboxyethyl)phosphine (10 mM, 0.055 mL, 0.551 μmol) and a solution of compound 23 (2.01 mg, 2.235 μmol) in DMSO (0.20 mL) at 22° C., and the reaction solution was placed in the water bath shaker, shaken and reacted at 22° C. for 3 h before the reaction was stopped.

⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 12 (20 mg, 2.44 mg/mL, yield: 91%).
N^{a-1} was found to be 4.17 as detected by RP.

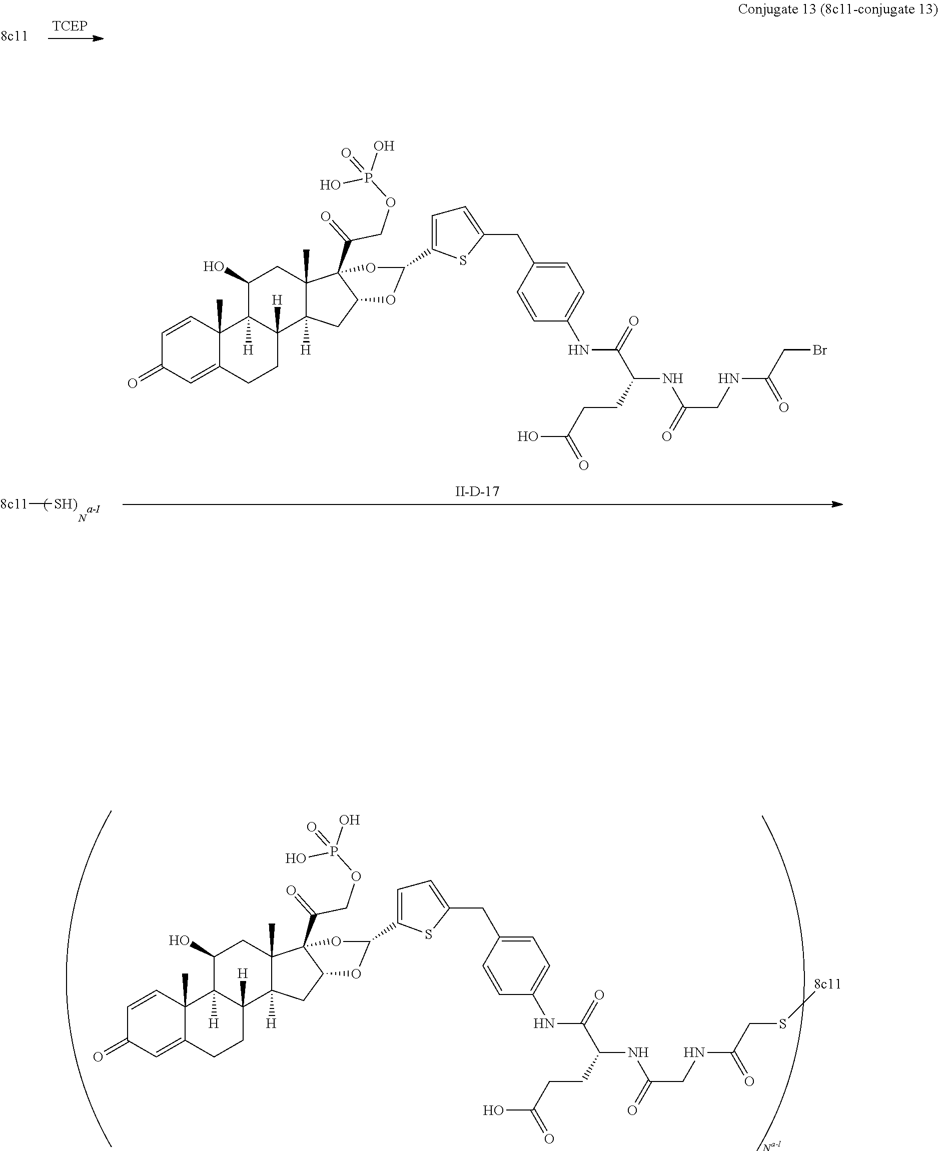

To an aqueous NaOAc buffer (0.05 M aqueous NaOAc buffer at pH=5.5, containing 0.51 mM EDTA and 20.35 mM sodium citrate; 6.0 mL, 6 mg/mL, 0.243 μmol) of antibody 8c11 were added the prepared aqueous solution of tris(2-carboxyethyl)phosphine (10 mM, 0.097 mL, 0.972 μmol) and a solution of compound 24 (3.51 mg, 3.645 μmol) in DMSO (0.35 mL) at 22° C., and the reaction solution was placed in the water bath shaker, shaken and reacted at 22° C. for 3 h before the reaction was stopped.

⅓ volumes of 800 mM aqueous sodium citrate buffer (pH=5.5) and 10% volumes of aqueous sucrose solution (60%) were added to the reaction solution, and the mixed solution was desalted and purified by using a Zeba desalting spin column (40K MWCO) (eluent: 0.05 M aqueous sodium citrate buffer at pH 6.0, containing 6% sucrose) to give conjugate 13 (29 mg, 4.69 mg/mL, yield: 80%).
N^{a-1} was found to be 4.31 as detected by RP.

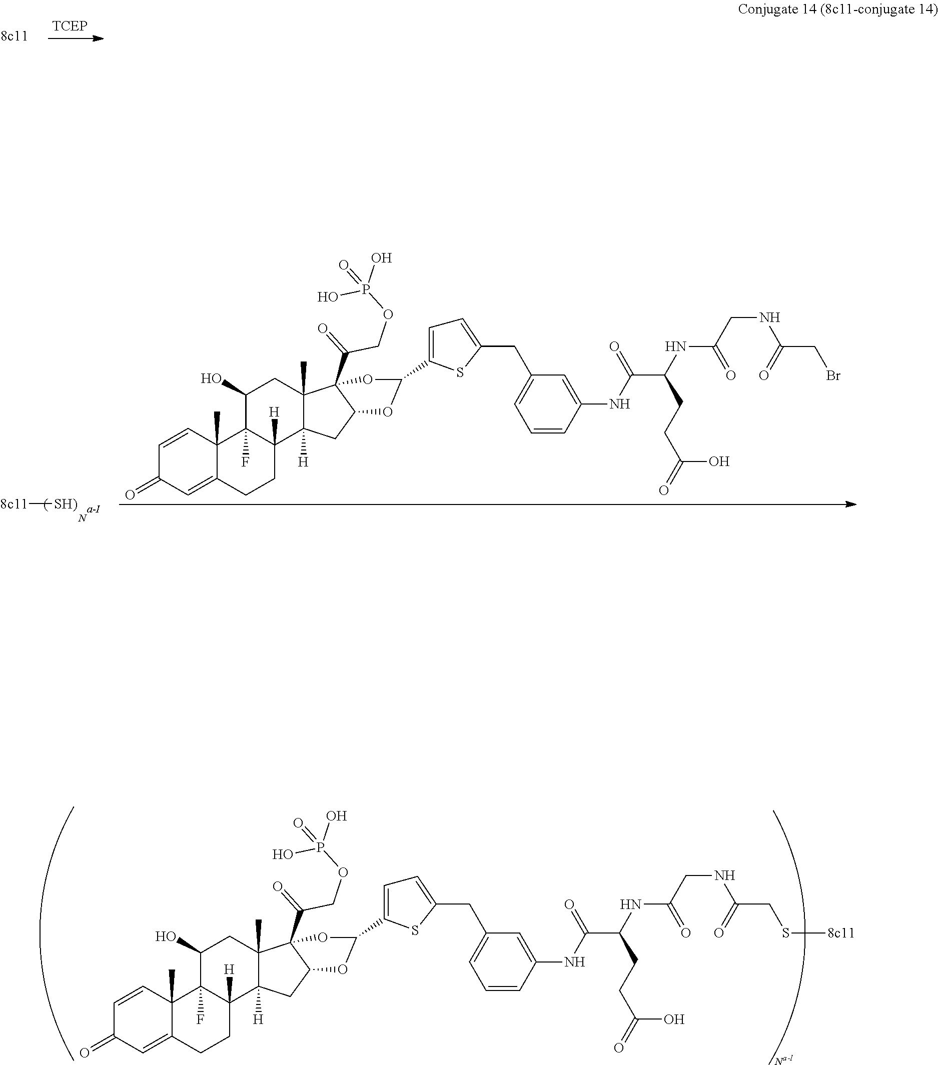

To an aqueous NaOAc buffer (0.05 M aqueous NaOAc buffer at pH=5.5, containing 0.51 mM EDTA and 20.35 mM sodium citrate; 6.0 mL, 6 mg/mL, 0.243 μmol) of antibody 8c11 were added the prepared aqueous solution of tris(2-carboxyethyl)phosphine (10 mM, 0.10 mL, 1.008 μmol) and a solution of compound 25 (3.57 mg, 3.645 μmol) in DMSO (0.36 mL) at 22° C., and the reaction solution was placed in the water bath shaker, shaken and reacted at 22° C. for 5 h before the reaction was stopped.

⅓ volumes of 800 mM aqueous sodium citrate buffer (pH=5.5) and 10% volumes of aqueous sucrose solution (60%) were added to the reaction solution, and the mixed solution was desalted and purified by using a Zeba desalting spin column (40K MWCO) (eluent: 0.05 M aqueous sodium citrate buffer at pH 6.0, containing 6% sucrose) to give conjugate 14 (29 mg, 3.09 mg/mL, yield: 80%).
N^{a-1} was found to be 4.29 as detected by RP.

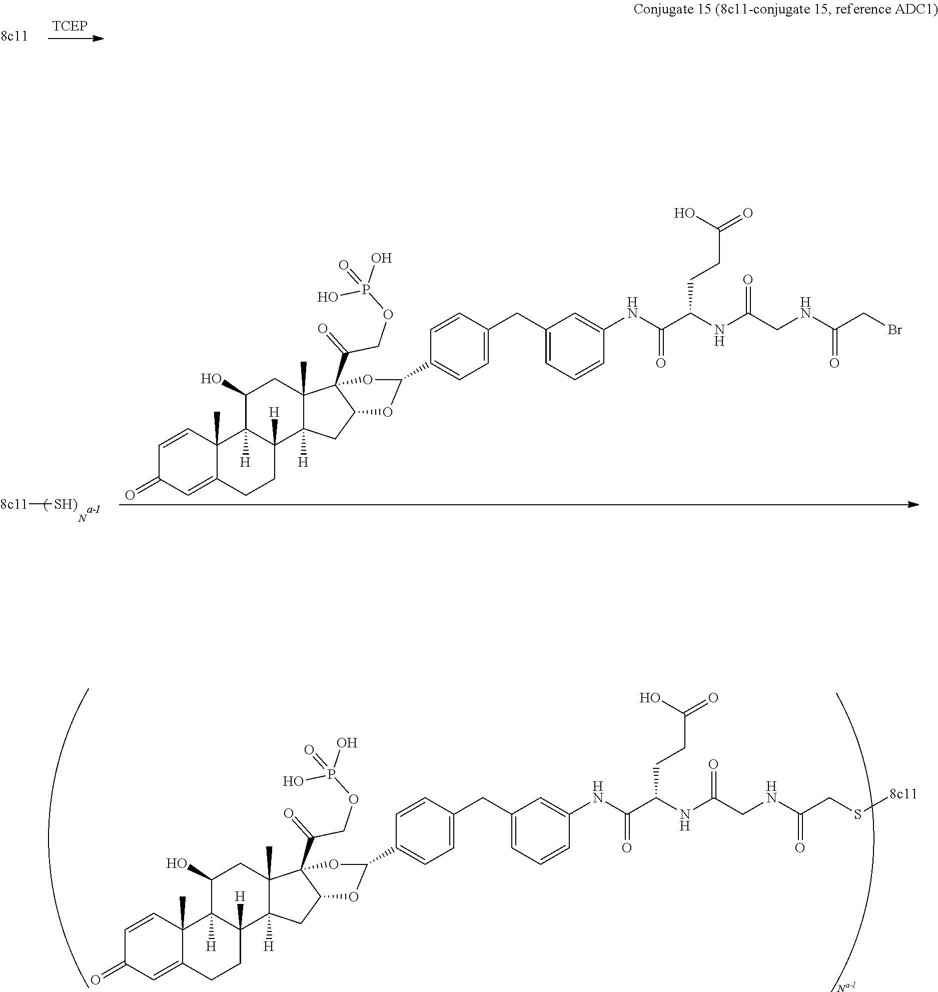

To an aqueous NaOAc buffer (0.05 M aqueous NaOAc buffer at pH=5.5, containing 0.51 mM EDTA and 20.35 mM sodium citrate; 11.0 mL, 6.0 mg/mL, 0.446 μmol) of antibody 8c11 were added the prepared aqueous solution of tris(2-carboxyethyl)phosphine (10 mM, 0.174 mL, 1.74 μmol) and a solution of compound 16 (6.40 mg, 6.69 μmol) in DMSO (0.64 mL) at 22° C., and the reaction solution was placed in the water bath shaker, shaken and reacted at 22° C. for 4.5 h before the reaction was stopped.

⅓ volumes of 800 mM aqueous sodium citrate buffer (pH=5.5) and 10% volumes of aqueous sucrose solution (60%) were added to the reaction solution, and the mixed solution was desalted and purified by using a Zeba desalting spin column (40K MWCO) (eluent: 0.05 M aqueous sodium citrate buffer at pH 6.0, containing 6% sucrose) to give conjugate 15 (54 mg, 3.18 mg/mL, yield: 82%).
N^{a-1} was found to be 4.69 as detected by RP.

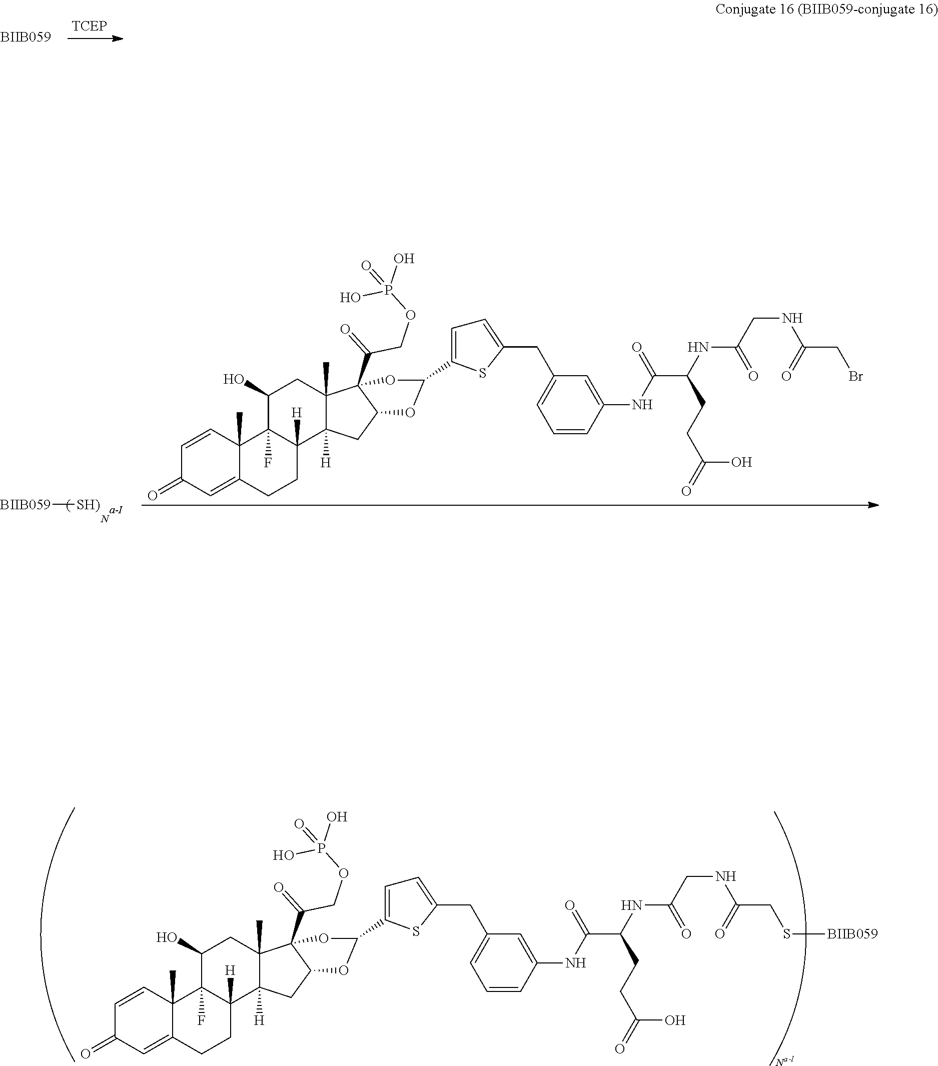

To an aqueous PB buffer (0.05 M aqueous PBS buffer at pH=7.4; 0.99 mL, 10.14 mg/mL, 0.068 μmol) of antibody BIIB059 was added the prepared aqueous solution of tris(2-carboxyethyl)phosphine (10 mM, 0.025 mL, 0.252 μmol) at 22° C., and the reaction solution was placed in a water bath shaker, shaken and reacted at 22° C. for 3 h before the reaction was stopped. Stock solution 1 and stock solution 2 were added such that the buffer system contained 19.7 mM sodium succinate and 0.39 mM EDTA.

Compound 25 (1.00 mg, 1.02 μmol) was dissolved in 0.10 mL of DMSO, the resulting solution was added to the above solution, and the mixed solution was placed in the water bath shaker, shaken and reacted at 22° C. for 2 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 16 (9.68 mg, 3.12 mg/mL, yield: 97%).
N^{a-1} was found to be 3.15 as detected by LC-MS.

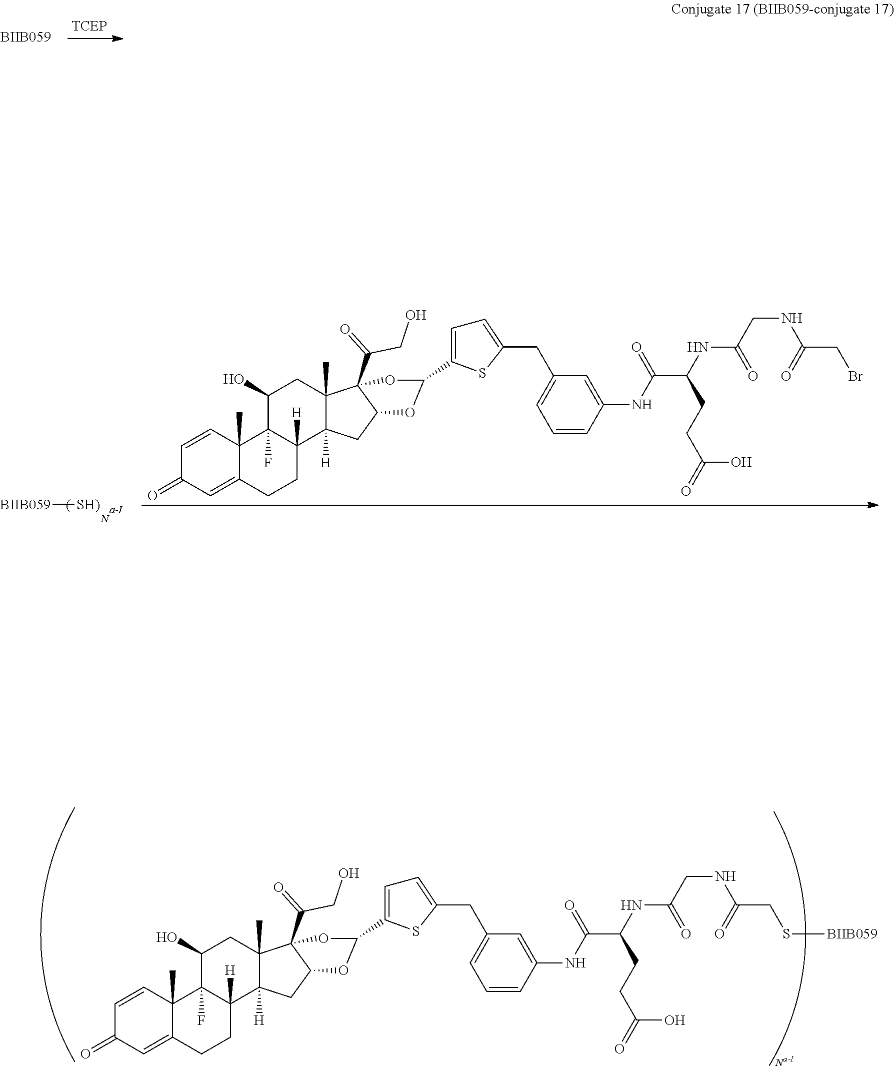

To an aqueous PB buffer (0.05 M aqueous PBS buffer at pH=7.4; 0.99 mL, 10.14 mg/mL, 0.068 μmol) of antibody BIIB059 was added the prepared aqueous solution of tris(2-carboxyethyl)phosphine (10 mM, 0.025 mL, 0.252 μmol) at 22° C., and the reaction solution was placed in a water bath shaker, shaken and reacted at 22° C. for 3 h before the reaction was stopped. Stock solution 1 and stock solution 2 were added such that the buffer system contained 19.7 mM sodium succinate and 0.39 mM EDTA.

Compound 23 (0.92 mg, 1.02 μmol) was dissolved in 0.09 mL of DMSO, the resulting solution was added to the above solution, and the mixed solution was placed in the water bath shaker, shaken and reacted at 22° C. for 2 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 17 (9.76 mg, 3.34 mg/mL, yield: 98%).
N^{a-1} was found to be 3.13 as detected by LC-MS.

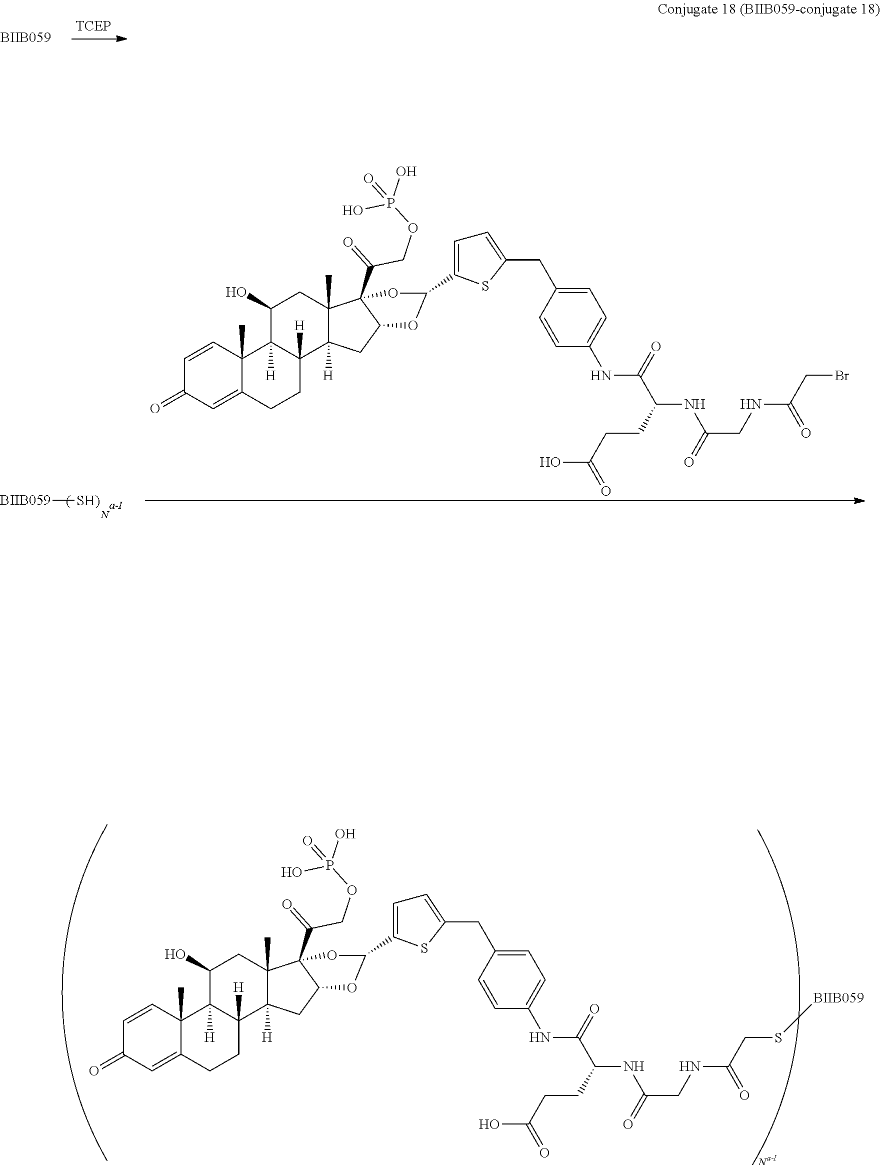

To an aqueous PB buffer (0.05 M aqueous PBS buffer at pH=7.4; 0.98 mL, 10.17 mg/mL, 0.068 μmol) of antibody BIIB059 was added the prepared aqueous solution of tris(2-carboxyethyl)phosphine (10 mM, 0.024 mL, 0.238 μmol) at 22° C., and the reaction solution was placed in a water bath shaker, shaken and reacted at 22° C. for 3 h before the reaction was stopped. Stock solution 1 and stock solution 2 were added such that the buffer system contained 19.7 mM sodium succinate and 0.39 mM EDTA.

Compound 24 (0.98 mg, 1.02 μmol) was dissolved in 0.10 mL of DMSO, the resulting solution was added to the above solution, and the mixed solution was placed in the water bath shaker, shaken and reacted at 22° C. for 2 h before the reaction was stopped. ⅓ volumes of 100 mM aqueous histidine buffer (pH=5.5) were added to the reaction solution, and the mixed solution was desalted and purified by using a Zeba desalting spin column (40K MWCO) (eluent: 0.02 M aqueous histidine buffer at pH 5.5) to give conjugate 18 (9.68 mg, 3.086 mg/mL, yield: 97%).
N^{a-1} was found to be 4.16 as detected by LC-MS.

### Example 3: Activity Assay of Glucocorticoid Receptor Reporter Gene GRE

### 1. Cell Culturing

HEK293 cells were cultured in DMEM+10% FBS medium in a carbon dioxide incubator humidified at 37° C. with 5% carbon dioxide.

### 2. Plasmid Transfection and Cell Plating

HEK293 cells were digested with trypsin, and the HEK293 cell resuspension was adjusted to 200,000 cell/mL in medium (96 well plate: 100 μL/well).

Transfection reagent mixtures were prepared according to Tables 5a and 5b below, mixed well and left to stand at room temperature for 20 min.

TABLE 5a

| Experimental transfection material | Volume (μL) |
|---|---|
| pGL5 plasmid (100 ng/μL) | 25 |
| pBIND-GR (100 ng/μL) | 25 |
| Opti-MEM medium | 250.0 |
| P3000 | 10.00 |
| Opti-MEM medium | 250.0 |
| Lipofectamine | 7.5 |

TABLE 5b

| Experimental transfection material | Volume (μL) |
|---|---|
| PLvx-EF1a-hBDCA2-CMV-hFcεRIγ- | 50 |
| IRES-puro (100 ng/μL) | |
| pGL5 plasmid (100 ng/μL) | 25 |
| pBIND-GR (100 ng/μL) | 25 |
| Opti-MEM medium | 250.0 |
| P3000 | 10.00 |
| Opti-MEM medium | 250.0 |
| Lipofectamine | 7.5 |

The prepared transfection mixtures were added to 10 mL of the cell density-adjusted resuspension, mixed well by turning the mixtures upside down, and seeded into a 96-well plate at a volume of 100 μL/well.

The 96-well plate was incubated in a humidified incubator at 37° C. with 5% carbon dioxide for 24 h.

### 3. Drug Treatment

210 μM of a solution of dexamethasone (purchased from the Sphsine) in DMSO was prepared and serially diluted at 3.3× to obtain 10 concentrations. 5 μL of the above dexamethasone in DMSO with each concentration was added into 45 μL of DMEM+10% FBS respectively and mixed evenly to obtain dexamethasone dilutions with 10 concentrations.

2.1 μM of solutions of the compounds of the present application or the compounds of the reference examples (diluted with culture medium) were prepared and serially diluted at 3.3× to obtain test compound dilutions with 10 concentrations.

5 μL of dexamethasone dilution or test compound dilution was added into each well (100 μL of cells had been added). 5 μL of 10% DMSO medium was added into Min wells, and 5 μL 21 μM dexamethasone (dissolved in medium, 10% DMSO) was added into Max wells.

The test plate was placed back into the incubator and incubated for 24 h.

### 4. Luciferase Assay

The test plate was detected using the Dual-Glo Luciferase Assay System kit from Promega, and the fluorescence signal of Firefly Luciferase and the fluorescence signal of Renilla Luciferase were read using Enspire, respectively.

### 5. Data Processing

The final measurement value was a normalized value “F/R” obtained by dividing the Firefly Luciferase fluorescent signal by the Renilla Luciferase fluorescent signal.

The data were copied and pasted into Excel, and the activation rate was obtained through an equation.

$$Rate of activation ( \% ) = \frac{Final measurement value - Min mean}{Max mean - Min mean} \times 100 \%$$

The data were imported into MS Excel and subjected to curve fitting by using XLFit excel add-in version 5.4.0.8 to obtain an EC₅₀ value.

$$Y = Bottom + ( Top - Bottom ) / ( 1 + ( EC 50 / X ) \hat{} HillSlope \qquad Formula$$

Y is the agonism and X is the concentration of the compound.

Table 6 shows that the compounds of the present application exhibited stronger glucocorticoid receptor agonistic activity.

TABLE 6
Measurement 1 of glucocorticoid receptor reporter gene GRE activity

| Compound ID | EC₅₀ (nM) |
|---|---|
| Dexamethasone | 1.77 |
| Compound 10 (Reference | >1000.00 |
| Compound P1) | |
| Compound 19 (Reference | >1000.00 |
| Compound P2) | |
| Compound 20 (Reference | >1000.00 |
| Compound P3) | |
| Compound 28 (Reference | 10.04 |
| Compound P4) | |
| Compound 28S (Reference | >1000.00 |
| Compound P4S) | |
| Compound 1 | 0.19 |
| Compound 2 | 0.15 |
| Compound 7 | 0.13 |
| Compound 8 | 0.07 |
| Compound 11 | 0.37 |
| Compound 15 | 0.63 |
| Compound 16 | 0.30 |
| Compound 17 | 0.33 |
| Compound 18 | 0.28 |

### Example 9: GR Luciferase Reporter Gene Elution Experiment

### Objective

The compounds of the present application were evaluated for their ability to continue activation upon binding to GR.

### Procedures

### 1. Cell Culture

HEK293 cells were cultured in DMEM+10% FBS medium in a carbon dioxide incubator humidified at 37° C. with 5% carbon dioxide.

### 2. Plasmid Transfection and Cell Plating

1) HEK293 cells were digested with trypsin, and the HEK293 cell resuspension was adjusted to 200,000 cell/mL in medium (96 well plate: 100 μL/well).
2) Transfection reagent mixtures were prepared according to the table below, mixed well and left to stand at room temperature for 20 min.

TABLE 7

| Experimental transfection material | Volume (μL) |
|---|---|
| pGL5 plasmid (100 ng/μL) | 25 |
| pBIND-GR (100 ng/μL) | 25 |
| Opti-MEM medium | 250.0 |
| P3000 | 10.00 |
| Opti-MEM medium | 250.0 |
| Lipofectamine | 7.5 |

3) The prepared transfection mixtures were added to 10 mL of the cell density-adjusted resuspension, mixed well by turning the mixtures upside down, and seeded into a 96-well plate at a volume of 100 μL/well.
4) The 96-well plate was incubated in a humidified incubator at 37° C. with 5% carbon dioxide for 24 h.

### 3. Drug Treatment

1) 210 μM dexamethasone in DMSO was prepared and serially diluted at 3× to obtain 10 concentrations. 5 μL of the above dexamethasone in DMSO with each concentration was added into 45 μL of DMEM+10% FBS respectively and mixed evenly to obtain dexamethasone dilutions with 10 concentrations.
2) A DMSO solution of the test compound at 210-fold final concentration was prepared and serially diluted at 3× to obtain dilutions of the test compound with 10 concentrations. 5 μL of the above DMSO solution with each concentration was added into 45 μL of DMEM+10% FBS respectively and mixed evenly to obtain dilutions with 10 concentrations.
3) 5 μL of dexamethasone dilution or test compound dilution was added into each well (100 μL of cells had been added). 5 μL of 10% DMSO medium was added into Min wells, and 5 μL 21 μM dexamethasone (dissolved in medium, 10% DMSO) was added into Max wells.
4) After the test plate was placed back into the incubator for incubation for a certain period of time (15 min/30 min/1 h/2 h/4 h), the supernatant was carefully and slowly removed with a pipette and added into the medium at 100 μL/well, then the supernatant was carefully and slowly removed again and finally added into the medium at 100 μL/well, followed by placement into the incubator for incubation for 24 h.

### 4. Luciferase Assay

The test plate was detected using the Dual-Glo Luciferase Assay System kit from Promega, and the fluorescence signal of Firefly Luciferase and the fluorescence signal of Renilla Luciferase were read using Enspire, respectively.

5. Data Processing
1) The final measurement value was a normalized value “F/R” obtained by dividing the Firefly Luciferase fluorescent signal by the Renilla Luciferase fluorescent signal.
2) The data were copied and pasted into Excel, and the activation rate was obtained through an equation.

$$Rate of activation ( \% ) = \frac{Final measurement value - Min mean}{Max mean - Min mean} \times 100 \%$$

3) The data were imported into MS Excel and subjected to curve fitting by using XLFit excel add-in version 5.4.0.8 to obtain an EC₅₀ value.

$$Y = Bottom + ( Top - Bottom ) / ( 1 + ( EC 50 / X ) \hat{} HillSlope \qquad Formula$$

Y is the agonism and X is the concentration of the compound.

### Experimental Results

The results in Table 8 show that the compounds of the present application bound tightly to the glucocorticoid receptor, were less likely to be eluted, and had a longer lasting activation of the glucocorticoid receptor.

TABLE 8
GR luciferase reporter gene elution experimental results

| | Elution time (Act %) | | | | |
|---|---|---|---|---|---|
| Compound No. | 15 min | 30 min | 1 h | 2 h | 4 h |
| Dexamethasone | 9.4 | 10.0 | 7.2 | 6.2 | 31.9 |
| Compound 16 | 32.9 | 29.2 | 28.7 | 23.8 | 32.2 |
| Compound 17 | 1.7 | 0.7 | 1.3 | 2.9 | 9.7 |

### Example 4: Inhibitory Effect of Compounds of the Present Invention on R848-Induced Release of IFNα and TNFα from Human Peripheral Blood Monocytes

### Objective

The compounds of the present application were detected for their inhibitory effect on the production of peripheral blood monocytes. The peripheral blood monocytes were treated in vitro with the ligand-drug conjugates at different concentrations, and IFNα and TNFα produced by the peripheral blood monocytes were quantitatively detected after a certain time. The compounds of the present invention were evaluated for in-vitro activity.

### Test Method

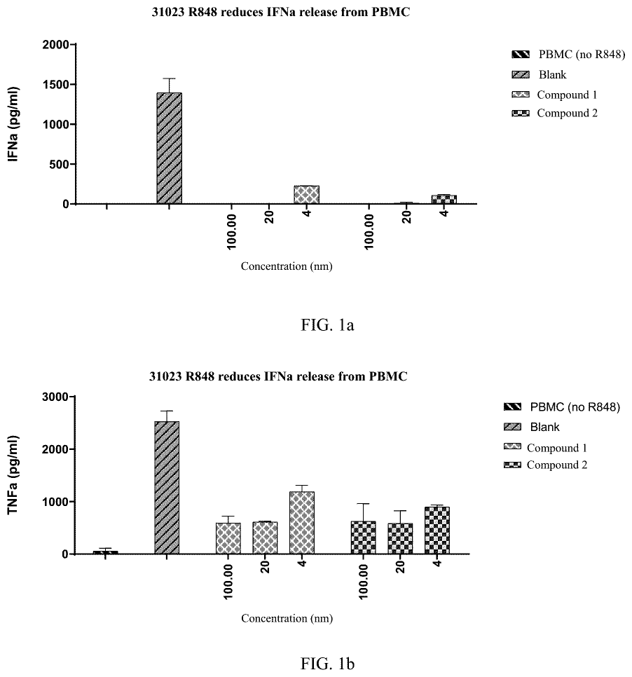

The cryopreserved human PBMC cells were resuspended in a complete medium and counted, and the cell suspension was adjusted to 2.5×10\hat{ }6 cells/mL with the culture medium. After the suspension was centrifuged to collect the cells the next day, the cells were resuspended using fresh culture medium. Test compounds were diluted to 4-fold final concentration in cell culture medium, 50 μL of the compounds were added into a 96-well cell plate, and 100 μL of diluted cells (800,000 cells/well) were added into each well of the 96-well plate containing the compounds, followed by addition of 50 μL R848 to a final concentration of 5 μM. The cell plate was incubated at 37° C. with 5% CO₂ for 24 h. The supernatant was collected, and the TNF-α and IFN-α concentration levels in the supernatant were detected using an ELISA kit. TNF-α concentration assay was performed using Dakewe TNF-α ELISA kit (Cat #1117202), 100 μL of the serially-diluted cytokine standard was added into each well, 100 μL of the above supernatant diluted 10-fold was added into each well, and 100 μL of 1× dilution buffer R was added into each blank control well. 50 μL of biotinylated antibody working solution was added into each well. After mixing well, the plate was covered with a plate-sealing membrane and incubated at room temperature for 3 h. The plate was washed for 3 times: the liquid in each well was discarded, and 300 μL of 1× washing buffer working solution was added into each well; after 1 minute of residence, the liquid in the wells was discarded. The procedure was repeated for 3 times. 100 μL of Streptavidin-HRP working solution was added into each well. The plate was covered with the plate-sealing membrane and incubated at room temperature for 20 min. The plate was washed for 3 times, 100 μL of TMB liquid was added into each well, and the plate was incubated at room temperature (18-25° C.) in the dark for 25 min. The reaction was stopped by adding 100 μL stop solution rapidly to each well. The reading at a wavelength of 450 nm was detected by an M2e instrument within 10 min after termination. The results are shown in FIGS. 1a and 1b.

IFN-α concentration assay was performed using Dakewe IFN-α ELISA kit (Cat #1110012), 100 μL of the serially-diluted cytokine standard was added into each well, 100 μL of the above supernatant diluted 2-fold was added into each well, and 100 μL of 1× dilution buffer R was added into each blank control well. 50 μL of biotinylated antibody working solution was added into each well. After mixing well, the plate was covered with a plate-sealing membrane and incubated at room temperature for 3 h. The plate was washed for 3 times: the liquid in each well was discarded, and 300 μL of 1× washing buffer working solution was added into each well; after 1 minute of residence, the liquid in the wells was discarded. The procedure was repeated for 3 times. 100 μL of Streptavidin-HRP working solution was added into each well. The plate was covered with the plate-sealing membrane and incubated at room temperature for 20 min. The plate was washed for 3 times, 100 μL of TMB liquid was added into each well, and the plate was incubated at room temperature (18-25° C.) in the dark for 25 min. The reaction was stopped by adding 100 μL stop solution rapidly to each well. The reading at a wavelength of 450 nm was detected by an M2e instrument within 10 min after termination.

The contents of TNF-α and IFN-α were calculated from the OD450 values (Mean±standard deviation).

The results are shown in Tables 9-1 and 9-2 and FIGS. 1a and 1b.

TABLE 9-1
Inhibition of compounds on IFN-α secretion from
PBMC under R848 stimulation

| Concentration of the | | | |
|---|---|---|---|
| compound | Compound 2 | Compound 1 | Blank |
| 100 nM | 0 ± 0 | 0 ± 0 | 1394.2 ± 180.7 |
| 20 nM | 11.1 ± 11.1 | 0 ± 0 | |
| 4 nM | 108 ± 11 | 228.3 ± 0.2 | |

TABLE 9-2
Inhibition of compounds on TNF-α secretion from
PBMC under R848 stimulation

| Concentration of | | | |
|---|---|---|---|
| the compound | Compound 2 | Compound 1 | Blank |
| 100 nM | 623.3 ± 238.4 | 592.8 ± 92.1 | 2526.6 ± 143.7 |
| 20 nM | 584.4 ± 171.1 | 611.9 ± 10.7 | |
| 4 nM | 898.3 ± 27.7 | 1187.6 ± 87.4 | |

The results show that the compounds of the present application could affect the ability of peripheral blood monocytes to produce TNF-α and IFN-α. The ligand-drug conjugates of the present application can inhibit inflammation, and can be used for preventing and/or treating diseases and/or conditions such as inflammation.

### Example 5: Assay of Biological Activity in Fluorescein Isothiocyanate (FITC)-Induced Delayed-Type IV Hypersensitivity Mouse Model

ADC was evaluated in an acute contact hypersensitivity model in which a delayed type hypersensitivity (DTH) response (T cell drive) was used to trigger acute skin inflammation by the application of sensitizers (fluorescein isothiocyanate, FITC). The potency of the ligand-drug conjugates (ADCs) of the present application was measured by their ability to reduce ear swelling. Before the experiment began, a specified number of 7-9 week-old female Balb/c mice were selected, random grouping was performed according to the weight of mice, with 10 mice in each group, and the average weight values among different groups were ensured to be similar so as to reduce the difference among the groups. The day of grouping the mice was defined as day 0 of the experiment. After random grouping, all mice were shaved off abdominal hair under mild anesthesia with 5% isoflurane, and mice in the disease-induced group were sensitized firstly by applying 100 μL of 2% fluorescein isothiocyanate (FITC) solution to their abdomen, while mice in the disease-controlled group were applied with 100 μL of vehicle control solution (acetone:DBP, volume ratio 1:1) to their abdomen. After the solvent solution and the 2% FITC solution were absorbed and the abdominal skin was dry, the mice were placed back into the cage box after they were awake. On day 1 of the experiment, the procedure described above was repeated for a second 2% FITC sensitization.

All mice were weighed on day 5 of the experiment. According to the experimental design, the mice in the solvent control group or each treatment group were respectively injected with the sterile PBS solution or the specified administration solution in the abdominal cavity. 1 h after administering, all mice were tested for left and right ear thickness using Mitutoya Caliper under mild anesthesia with 5% isoflurane. After ear thickness measurement, all mice in the disease-induced group were coated with 10 μL of 1% FTIC solution on the right ear and 10 μL of vehicle control solution (acetone:DBP, volume ratio 1:1) on the left ear.

On day 6 of the experiment, 24 h after 1% FTIC excitation, all mice were tested for left and right ear thickness using Mitutoya Caliper under mild anesthesia with 5% isoflurane and mouse ear thickness change was calculated according to the following formula: (R−L)−(R0−L0), wherein R0 and L0 are right and left ear thickness before 1% FITC excitation, respectively, and R and L are right and left ear thickness after 24 h of 1% FITC excitation, respectively.

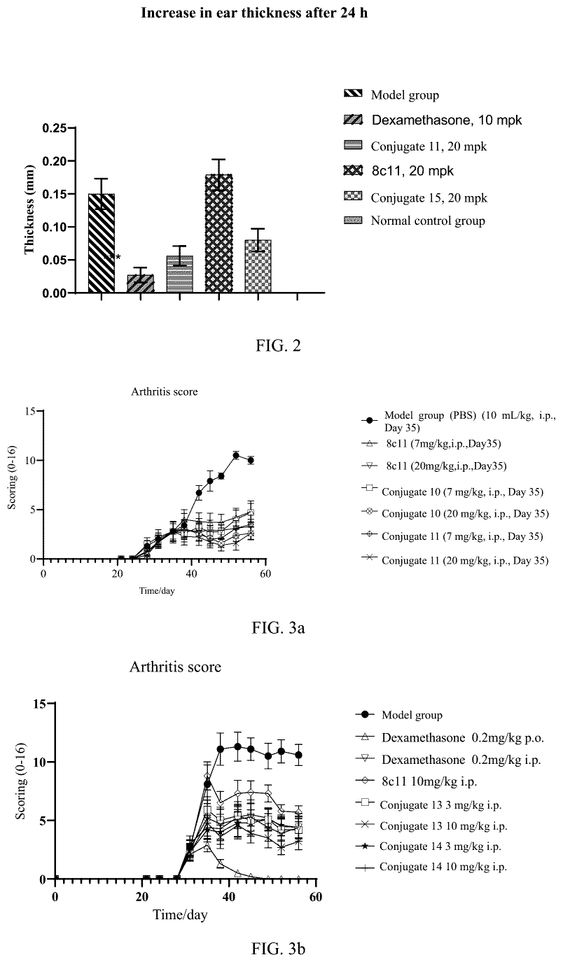

The results are shown in FIG. 2. The results show that conjugate 11, monoclonal antibody 8c11 and conjugate 15 (reference ADC-1) could inhibit fluorescein isothiocyanate (FITC)-induced delayed-type IV hypersensitivity and reduce mouse ear thickness, wherein the ear thickness is 0.056 mm, 0.179 mm and 0.08 mm, respectively; the inhibitory effect of conjugate 11 was significantly superior to that of monoclonal antibody 8c11 and conjugate 15 (reference ADC-1).

The ligand-drug conjugates (ADCs) of the present application had the ability to inhibit hypersensitivity, and the ligand-drug conjugates (ADCs) of the present application could be used for preventing and/or treating diseases and/or conditions of inflammation.

### Example 6: Assay of Biological Activity of Bovine Type II Collagen Mixed Adjuvant-Induced DBA/1 Mouse Arthritis Model

The potency of the ligand-drug conjugates (ADCs) of the present application was evaluated in a bovine collagen-induced arthritis (CIA) model.

Male DBA/1J mice were obtained from Jackson Labs. The mice were used at 6-8 weeks of age. All animals were given ad libitum access to food and water under a 12-hour light/dark cycle at constant temperature and humidity. Body weight and condition were monitored and if >20% weight loss was exhibited, the animals were euthanized.

Bovine collagen (Chondrex, Inc. Cat: 20021) was dissolved in 0.1 M acetic acid and stored overnight in a 2-8 C refrigerator at a collagen concentration of 8 mg/mL, and an equal volume of CFA (complete Freund's adjuvant, Difco, Laurence, KS) was added into the collagen solution and emulsified in a high speed homogenizer to make an emulsion. On Day 0, animals in need of immunization were anesthetized with 2%-5% isoflurane and male DBA/J mice were immunized once with a single subcutaneous injection of 50 μL collagen/CFA emulsion at the tail root of 2-3 cm from the body. Three weeks later, namely on Day 21, the tail root was immunized twice with the same volume of collagen emulsion by the same injection method. After the second immunization, 120 animals with an average score of about 2 for the limb arthritis were selected and randomly grouped. The animals were treated by a single intraperitoneal injection of test reagent according to the experimental design after grouping. For the dexamethasone (Sphsine) treatment group, the drug was administered the day after grouping until the end of the trial. The extremities of each group were observed twice weekly for the onset of arthritis from the time of the second immunization, and the observation was continued until the end of the trial.

Scoring according to the degree of lesion (redness and swelling) was performed on a scale of 0-4 points, with a maximum score of 4 for each limb and a total maximum score of 16 for each animal's limbs. The scoring criteria are as follows: 0 points, no redness and swelling; 1 point, 1-2 red and swollen interphalangeal joints; 2 points, 3-4 red and swollen interphalangeal joints; 3 points, more than 4 red and swollen interphalangeal joints; 4 points, severe redness and swelling in toes or fingers to ankle joints or wrist joints. For joint swelling, ankle thickness of hind paw of each animal was measured with a screw micrometer before one immunization. Since the second immunization, measurements were performed 2 times per week, and changes in ankle thickness before and after the immunization were compared. After the trial was completed, all animals were anesthetized with 2-5% isoflurane and then euthanized by exsanguination.

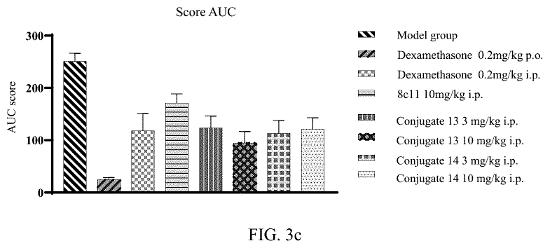

Score AUC: area under the curve for arthritis score. The results are shown in Tables 10-1 and 10-2 and FIGS. 3a-3c.

TABLE 10-1
Arthritis score

| | | Arthritis score | | | | | | | | | | |
|---|---|---|---|---|---|---|---|---|---|---|---|---|
| Group | | Day 21 | Day 24 | Day 28 | Day 31 | Day 35 | Day 38 | Day 42 | Day 45 | Day 49 | Day 52 | Day 56 |
| Model group (PBS) (10 | Mean | 0.00 | 0.00 | 1.30 | 2.00 | 2.80 | 3.40 | 6.70 | 7.90 | 8.40 | 10.50 | 10.00 |
| mL/kg, i.p., Day 35) | SEM | 0.00 | 0.00 | 0.56 | 0.63 | 0.95 | 1.21 | 0.75 | 1.05 | 0.31 | 0.40 | 0.39 |
| 8c11(7 mg/kg, i.p., Day 35) | Mean | 0.00 | 0.00 | 0.70 | 1.80 | 2.80 | 4.00 | 3.80 | 3.70 | 3.70 | 4.20 | 4.80 |
| | SEM | 0.00 | 0.00 | 0.40 | 0.61 | 1.02 | 1.01 | 0.88 | 0.94 | 0.82 | 0.93 | 1.09 |
| 8c11 (20 mg/kg, i.p., Day 35) | Mean | 0.00 | 0.00 | 0.70 | 1.70 | 2.70 | 2.90 | 2.80 | 2.90 | 2.90 | 3.10 | 3.30 |
| | SEM | 0.00 | 0.00 | 0.50 | 0.65 | 1.09 | 0.92 | 0.94 | 1.03 | 0.78 | 0.75 | 0.73 |
| Conjugate 10 (7 mg/kg, | Mean | 0.00 | 0.00 | 1.50 | 2.20 | 2.80 | 2.60 | 3.20 | 2.70 | 2.80 | 4.00 | 4.70 |
| i.p., Day 35) | SEM | 0.00 | 0.00 | 0.65 | 0.77 | 1.01 | 0.90 | 0.90 | 0.65 | 0.66 | 0.94 | 0.94 |
| Conjugate 10 (20 mg/kg, | Mean | 0.00 | 0.00 | 0.80 | 1.70 | 2.80 | 3.10 | 2.50 | 2.10 | 1.60 | 2.40 | 2.60 |
| i.p., Day 35) | SEM | 0.00 | 0.00 | 0.42 | 0.56 | 0.79 | 0.89 | 0.65 | 0.59 | 0.43 | 0.64 | 0.62 |
| Conjugate 11 (7 mg/kg, | Mean | 0.00 | 0.00 | 0.40 | 1.80 | 2.80 | 3.00 | 2.70 | 2.00 | 2.10 | 3.10 | 3.50 |
| i.p., Day 35) | SEM | 0.00 | 0.00 | 0.27 | 0.55 | 0.95 | 0.94 | 0.91 | 0.70 | 0.78 | 1.00 | 1.15 |
| Conjugate 11 (20 mg/kg, | Mean | 0.00 | 0.00 | 0.40 | 1.70 | 2.80 | 2.30 | 2.20 | 1.70 | 1.40 | 1.60 | 2.60 |
| i.p., Day 35) | SEM | 0.00 | 0.00 | 0.22 | 0.56 | 1.01 | 0.88 | 0.84 | 0.58 | 0.58 | 0.72 | 0.69 |

Mean: mean value, SEM: standard deviation.

TABLE 10-2
Arthritis score

| | | Arthritis score | | | | | | | | | | | | |
|---|---|---|---|---|---|---|---|---|---|---|---|---|---|---|
| Group | | D 0 | D 21 | D 24 | D 28 | D 31 | D 35 | Day 38 | Day 42 | Day 45 | Day 49 | Day 52 | Day 56 | AUC |
| Model group | Mean | 0.00 | 0.00 | 0.00 | 0.00 | 2.70 | 8.10 | 11.10 | 11.30 | 11.10 | 10.50 | 10.90 | 10.60 | 251.15 |
| | SEM | 0.00 | 0.00 | 0.00 | 0.00 | 0.65 | 1.66 | 1.38 | 1.24 | 0.97 | 1.09 | 0.99 | 0.91 | 15.04 |
| Dexamethasone | Mean | 0.00 | 0.00 | 0.00 | 0.00 | 2.10 | 2.90 | 1.30 | 0.50 | 0.20 | 0.00 | 0.00 | 0.00 | 24.50 |
| 0.2 mg/kg p.o. | SEM | 0.00 | 0.00 | 0.00 | 0.00 | 0.50 | 0.57 | 0.37 | 0.27 | 0.20 | 0.00 | 0.00 | 0.00 | 4.14 |
| 8c11 10 mg/kg | Mean | 0.00 | 0.00 | 0.00 | 0.00 | 2.50 | 8.80 | 6.50 | 7.30 | 7.40 | 7.30 | 5.80 | 5.70 | 171.00 |
| | SEM | 0.00 | 0.00 | 0.00 | 0.00 | 0.54 | 1.21 | 0.98 | 1.14 | 0.98 | 0.73 | 0.55 | 0.56 | 17.60 |
| Conjugate 13 3 | Mean | 0.00 | 0.00 | 0.00 | 0.00 | 2.30 | 5.90 | 5.00 | 5.40 | 5.00 | 5.10 | 4.20 | 4.20 | 123.55 |
| mg/kg | SEM | 0.00 | 0.00 | 0.00 | 0.00 | 0.50 | 1.13 | 0.97 | 0.98 | 0.92 | 0.99 | 0.99 | 0.70 | 22.63 |
| Conjugate 13 10 | Mean | 0.00 | 0.00 | 0.00 | 0.00 | 2.20 | 4.80 | 3.60 | 4.60 | 3.90 | 3.50 | 2.70 | 3.20 | 94.95 |
| mg/kg | SEM | 0.00 | 0.00 | 0.00 | 0.00 | 0.70 | 1.42 | 1.00 | 1.17 | 0.84 | 0.85 | 0.63 | 0.71 | 21.68 |
| Conjugate 14 3 | Mean | 0.00 | 0.00 | 0.00 | 0.00 | 2.30 | 4.20 | 4.00 | 4.80 | 4.80 | 5.10 | 4.60 | 4.40 | 113.10 |
| mg/kg | SEM | 0.00 | 0.00 | 0.00 | 0.00 | 0.65 | 1.04 | 1.04 | 1.16 | 1.13 | 0.94 | 0.88 | 0.90 | 24.42 |
| Conjugate 14 10 | Mean | 0.00 | 0.00 | 0.00 | 0.00 | 3.00 | 4.40 | 4.30 | 5.30 | 5.50 | 5.20 | 4.50 | 4.40 | 121.50 |
| mg/kg | SEM | 0.00 | 0.00 | 0.00 | 0.00 | 0.68 | 1.00 | 0.90 | 0.99 | 0.98 | 0.77 | 0.83 | 0.98 | 21.36 |

Mean: mean value, SEM: standard deviation.

The results show that the ligand-drug conjugates (ADCs) of the present application had the ability to reduce swelling of the hind paw of the mice, and could exhibit an extended duration of action of about 28 days, as compared to the blank agent, and had the ability to reduce the severity of arthritis and biological safety (e.g., blood safety).

### Example 7

### 7.1 Glucocorticoid Receptor Binding Assay

According to the manufacturer's protocol, small molecules were tested for glucocorticoid receptor (GR) binding using a Polarscreen T M glucocorticoid receptor assay kit, Red (ThermoFisher A 15898). Briefly, compounds were serially diluted in DMSO and transferred to an assay kit buffer at 1:10 dilution. The compounds were further diluted at 1: 5 in the assay kit buffer, and 10 μL of the diluted solutions were transferred to 384-well low-volume black-wall plates (Corning 4514). 5 μL of 4× Fluormone GS Red stock solution and 5 μL of 4× GR full-length stock solution were added to each well containing the test compound, and the plates were incubated at room temperature in the dark for 4 h. The fluorescence polarization (mP) was measured for each plate using an MD-5e multilable microplate reader, and the data were analyzed using four-parameter curve fitting to generate EC50 values.

### 7.2 Mineralocorticoid Receptor Cell Assay

According to the manufacturer's protocol, small molecules were tested for mineralocorticoid receptor (MR) agonist activity using a PathHunter® NHRPRO CHO-K1MR cell line (DiscoveRx, Cat. 93-0451C2). Briefly, cells were seeded into a 96 half area well plate (Costar, Cat. 3885) at 20,000 cells/well in a culture medium overnight at 37° C. The medium was removed and replaced with serially diluted small molecules in an assay medium (30 μL; 0.3% DMSO final concentration). The plate was incubated overnight at 37° C. The medium was removed and replaced with a detection reagent (DiscoveRx, Cat. 93-0001; 12 μL/well), and the plate was incubated at room temperature (RT) for 60 min. The luminescence was measured for each plate using an MD-5e multilable microplate reader, and the data were analyzed using four-parameter curve fitting to generate EC50 values.

### 7.3 Progesterone Receptor Binding Assay

Small molecules were tested for progesterone receptor (PR) binding using a modification scheme of a LanthaScreen® TR-FRET progesterone receptor coactivator assay (Thermofisher, Cat. A15903) in which the fluorescein-labeled coactivator peptide was replaced with Fluormone AL-Red (Thermofisher, Cat. PV4294) to improve assay signals. Briefly, compounds were serially diluted in DMSO and transferred to an assay buffer (Thermofisher, Cat. PV4301+5 mM DTT) at 1:10 dilution. 10 μL of the compounds were transferred to 96 half-area black well plates (Corning, Cat. 3694) in duplicate. 5 μL of PR-LBD protein (4 nM stock solution in the assay buffer; Thermofisher, Cat. P2899) was added to each well. In addition, 5 μL of a prepared mixture of Fluormone AL-Red (12 nM) and terbium-labeled anti-GST monoclonal antibody (mAb) (20 nM; Thermofisher, Cat. PV3550) in the assay buffer was also added to each well. The plates were incubated at room temperature (RT) for 2 h, and then the TR-FRET emission ratio was measured using an EnVision multilable microplate reader (Perkinelmer #2104-0010). The data were analyzed using four-parameter curve fitting to generate EC50 values.

### 7.4 Androgen Receptor Binding Assay

Small molecules were tested for androgen receptor (AR) binding using a modification scheme of a LanthaScreen® TR-FRET androgen receptor coactivator assay (Thermofisher, Cat. A15878) in which the fluorescein-labeled coactivator peptide was replaced with Fluormone AL-Red (Thermofisher, Cat. PV4294) to improve assay signals. Briefly, compounds were serially diluted in DMSO and transferred to an assay buffer (Thermofisher, Cat. PV4295+5 mM DTT) at 1:10 dilution. 10 μL of the compounds were transferred to 96 half-area black well plates (Corning, Cat. 3694) in duplicate. 5 μL of AR-LBD protein (5 nM stock solution in the assay buffer; Thermofisher, Cat. 3009) was added to each well. In addition, 5 μL of a prepared stock solution of Fluormone AL-Red (20 nM) and terbium-labeled anti-GST monoclonal antibody (mAb) (30 nM; Thermofisher, Cat. PV3550) in the assay buffer was also added to each well. The plates were incubated at room temperature (RT) for 6 h, and then the TR-FRET emission ratio was measured using an EnVision multilable microplate reader (Perkinelmer #2104-0010). The data were analyzed using four-parameter curve fitting to generate EC50 values.

### 7.5 Estrogen Receptor Binding Assay

Small molecules were tested for estrogen receptor (ER) a binding using a modification scheme of a LanthaScreen® TR-FRET estrogen receptor a coactivator assay (Thermofisher, Cat. A15885) in which the fluorescein-labeled coactivator peptide was replaced with Fluormone ES2 Green (Thermofisher, Cat. PV6045) to improve assay signals. Briefly, compounds were serially diluted in DMSO and transferred to an assay buffer (Thermofisher, Cat. PV4295+5 mM DTT) at 1:10 dilution. 10 μL of the compounds were transferred to 96 half-area black well plates (Corning, Cat. 3694) in duplicate. 5 μL of ER-LBD protein (5 nM stock solution in the assay buffer; Thermofisher, Cat. 4542) was added to each well. In addition, 5 μL of a prepared stock solution of Fluormone ES2Green (12 nM) and terbium-labeled anti-GST monoclonal antibody (mAb) (8 nM; Thermofisher, Cat. PV3550) in the assay buffer was also added to each well. The plates were incubated at room temperature (RT) for 4 h, and then the TR-FRET emission ratio was measured using an EnVision multilable microplate reader (Perkinelmer #2104-0010). The data were analyzed using four-parameter curve fitting to generate EC50 values.

The compounds of the present application had a relatively high affinity for glucocorticoid and relatively low affinities for mineralocorticoid receptors, progesterone receptors, androgen receptors and estrogen receptors.

### 7.6 GRE Reporter Gene Assay

Example HEK293 GRE cells were seeded into a 96-well tissue culture treated white plate (Costar: 3917) at 50,000 cells/well in 50 μL of assay medium (RPMI, 1% CSFBS, 1% L-glutamine, 1% sodium pyruvate and 1% MEAA). The compounds of the present application were serially four-fold diluted in 100% DMSO at an initial concentration of 100 μM. 2 μL of serially-diluted compounds were transferred into 248 μL of assay medium in a secondary dilution plate (1:125 dilution) such that the small-molecule compounds were further diluted in the assay medium. The cells were then treated with 25 μL of 1:125 diluted GR agonist compound or medium alone at a final initial concentration of 266.7 nM (1:3) and incubated at 37° C. with 5% CO₂ for 24 h. After 24 h of incubation, the cells were treated with 75 μL of Dual-Glo luciferase assay system (Promega-E2920) for 10 min and analyzed for luminescence using TopCount or MicroBeta2 (PerkinElmer). The results show that the compounds of the present application could effectively activate GRE reporter genes at a relatively low concentration.

### Example 8

### 8.1 GRE Activity Assay

### Generation of Human and Mouse Transmembrane TNF-α GRE Reporter Cell Lines

To generate the parental cell line, K562 cells were seeded at 500,000 cells per well into a 6-well culture dish (Costar: 3516) with 2 mL of complete growth medium (RPMI, 10% FBS, 1% L-glutamine, 1% sodium pyruvate and 1% MEMNEAA (an optional amino acid solution)) at 37° C. with 5% CO₂ within 24 h. The next day, 1.5 μg of pGL4.36 [Luc2P/MMTV/Hygro](Promega) and 3 μL of PLUS reagent (Invitrogen) were diluted in 244 μL of Opti-MEM (Gibco: 31985-070) and incubated at room temperature for 15 min. pGL4.36 [luc2P/MMTV/Hygro] vector contains the murine mammary tumor virus long terminal repeated sequence, which drives transcription of the luciferase reporter gene luc2P in response to activation of several nuclear receptors (e.g. glucocorticoid receptor and androgen receptor). After incubation, the diluted DNA solution was pre-incubated with 1:1 lipofectamine LTX solution (13.2 μL+256.8 μL of Opti-MEM) and incubated at room temperature for 25 min to form a DNA-lipofectamine LTX complex. After incubation, 500 μL of DNA-lipofectamine complex was added directly into the wells containing cells. The K562 cells were transfected at 37° C. with 5% CO₂ for 24 h. After incubation, the cells were washed with 3 mL of PBS and selected for two weeks with complete growth medium containing 125 μg/mL hygromycin B. “K562pGL4.36 [Luc2P/MMTV/Hygro]_pGL4.75 [hRLuc/CMV]” cells were generated.

To generate murine transmembrane TNF-α GRE reporter cells, the parent cells, K562pGL4.36 [Luc2P/MMTV/Hygro]_pGL4.75[hRLuc/CMV], were seeded at 500,000 cells per well into a 6-well culture dish containing 2 mL of complete growth medium (RPMI, 10% FBS, 1% L-glutamine, 1% sodium pyruvate and 1% MEMNEAA) at 37° C. with 5% CO₂ within 24 h. The next day, 3 μg of mFL_TNFα DNA encoding unlabeled mouse TNF and 3 μL of PLUS reagent (Invitrogen: 10964-021) were diluted in 244 μL of Opti-MEM (Gibco: 31985-070) and incubated at room temperature for 15 min. After incubation, the diluted DNA solution was pre-incubated with 1:1 lipofectamine LTX solution (13.2 μL+256.8 μL of Opti-MEM) and incubated at room temperature for 25 min to form a DNA-lipofectamine LTX complex. After incubation, 500 μL of DNA-lipofectamine complex was added directly into the wells containing cells. The parent K562pGL4.36[Luc2P/MMTV/Hygro]_pGL4.75[hRLuc/CMV] cells were transfected at 37° C. with 5% CO₂ for 24 h. After incubation, the cells were washed with 3 mL of PBS and selected for two weeks with complete growth medium containing 125 μg/mL hygromycin B (Invitrogen: 10687-010) and 250 μg/mL G418 (Gibco: 10131-027). “K562 mouse FL-TNFαGRE (pGL4.36[luc2P/MMTV/Hygro])” cells were generated.

To generate the human transmembrane TNF-α GRE reporter cell line, the parent cell K562pGL4.36[Luc2P/MMTV/Hygro]_pGL4.75[hRLuc/CMV] was transfected with the hTNF61-12C-MycpcDNA3.1(−) plasmid construct. The plasmid was pcDNA3.1 (Thermo Fisher, Cat. V79020), which encodes tace-resistant transmembrane TNF. (See PerezC et al., Cell 63(2): 251-8 (1990) which discusses tace-resistant transmembrane TNF). “K562 human TNF61-12GRE (pGL4.36[luc2P/MMTV/Hygro])” cells were generated. These cell lines were then used in the TNF-α reporter assay described in the examples below.

K562 parent GRE cells (“K562pGL4.36 [Luc2P/MMTV/Hygro]_pGL4.75[hRLuc/CMV]” cells), and GRE-mouse TNF-α cells (“K562 mouse FL-TNF-α GRE(pGL4.36[luc2P/MMTV/Hygro])” cells) or GRE-human TNF-α cells (“K562 human TNF61-12GRE (pGL4.36[luc2P/MMTV/Hygro])” cells) were seeded onto a 96-well plate at 50,000 cells per well. The ligand-drug conjugates (ADCs) of the present application were serially diluted at 3× in media and added into the above 96-well plate and incubated at 37° C. with 5% CO₂ for 48 h. Luminescence was analyzed after treatment with the luciferase assay system. The data were analyzed using four-parameter curve fitting to generate EC₅₀ values. The maximum % activation was normalized to 100 nM dexamethasone. The ligand-drug conjugates (ADCs) of the present application measured in-vitro activity in a mouse transmembrane TNFα GRE reporter assay; and the ligand-drug conjugates (ADCs) of the present application measured in-vitro activity in the human transmembrane TNFα GRE reporter assay.

The results show that the ligand-drug conjugates (ADCs) of the present application had the ability to affect the level of cellular GRE activation, and that the ligand-drug conjugates (ADCs) of the present application could affect the level of glucocorticoid-mediated signaling pathway activation.

### 8.2 Lipopolysaccharide (LPS) Induced Cytokine Release and Downstream Signal Detection

Primary human peripheral blood monocytes (PBMCs) were washed in 50 mL PBS, resuspended in FBS (fetal bovine serum) with 5% DMSO, divided in equal and cryopreserved in liquid nitrogen until use. The PBMCs were thawed, resuspended in cell culture medium (e.g., RPMI cell culture medium) with 2% FBS and 1% penicillin streptomycin, and seeded onto a 96-well plate. The cells were then incubated with different concentrations of ADCs at 37° C. with 5% CO₂ for 4 h. The cells were treated with a stimulant such as lipopolysaccharides (LPS) at a certain concentration for a certain time. The plate was then spun at 1000 rpm for 5 min, 100 μL of the supernatant culture medium was transferred directly into another 96-well plate, and the concentrations of cytokines such as IL-6 and IL-1β and phosphorylated STAT1 levels were analyzed. The results show that the ligand-drug conjugates (ADCs) of the present application had the ability to affect the release of cytokines, such as IL-6 and IL-1 β, from PBMC cells, and the ability to phosphorylate STAT1.

### 8.3 Assay of Biological Activity in Contact Hypersensitivity Model

ADC was evaluated in an acute contact hypersensitivity model in which a delayed type hypersensitivity (DTH) response (T cell drive) was used to trigger acute skin inflammation by the application of sensitizers (fluorescein isothiocyanate, FITC). The potency of the ligand-drug conjugates (ADCs) of the present application was measured by their ability to reduce ear swelling. Biomarkers, including, for example, steroids, corticosterone and procollagen type 1 N-terminal propeptide (P1NP) were detected to evaluate the effect of the ligand-drug conjugates (ADCs) of the present application on the hypothalamus-pituitary-adrenal (HPA) axis and bone turnover (bone resorption and bone formation).

### (1) Ear Swelling Detection

On day 0, mice were placed under general anesthesia and the abdomen was shaved. The mice were sensitized by intraperitoneal injection of 400 μL of FITC solution (1.5% solution in 1:1 acetone:DBp (dibutyl phthalate)) using a micropipette. After 6 days, the mice were administered (solvents for the control group or ligand-drug conjugates of the present application for the treatment group) 1 h prior to ear injection of FITC. For the ear injection, the mice were placed under general anesthesia and re-sensitized by injection of 20 μL of FITC in the right ear. After 24 h of re-sensitization, the ear thickness was measured with a vernier caliper under general anesthesia in the mice. The differences between sensitized ears and non-sensitized ears were calculated.

The results show that the ligand-drug conjugates (ADCs) of the present application had the ability to affect the skin swelling of the ears, and that the ligand-drug conjugates (ADCs) of the present application could be used for preventing and/or treating diseases and/or conditions of inflammation.

### (2) Quantitative Detection of Released Free Steroids and Endogenous Corticosterone as Well as Plasma P1NP was Performed.

72 h after ear sensitization, the mice were intraperitoneally injected with ACTH (adrenocorticotropic hormone) at 1 mpk (mg/kg) and bled at the end of 30 min after ACTH administration. Plasma was collected and analyzed for procollagen type 1 N-terminal propeptide (P1NP), corticosterone, free steroids and macromolecule levels.

Calibration curves of steroids were prepared in mouse plasma at various (e.g., 8) concentration levels with a final concentration of 0.03 nM to 0.1 μM. A calibration curve of corticosterone was prepared in 70 mg/mL bovine serum albumin solution in PBS buffer with a final corticosterone concentration ranging from 0.3 nM to 1 μM. A solution of 160 μL of MeCN (acetonitrile) containing 0.1% formic acid was added to 40 μL of the test plasma sample or calibration standard. The supernatant was diluted with distilled water, and 30 μL of the final sample solution was injected for LC/MS analysis.

After trypsin digestion, quantification of plasma procollagen type 1 N-terminal propeptide (P1NP) was performed on an LCMS platform. As a specific type I collagen precipitation indicator, P1NP levels reflected the status of osteoblast activity and bone formation. Plasma samples were partially precipitated and fully reduced by the addition of a MeCN/0.1 M ammonium bicarbonate/DTT (dithiothreitol) mixture. The supernatant was collected and alkylated by the addition of iodoacetic acid. The alkylated proteins were digested by trypsin, and the resulting tryptic peptides were analyzed by LC/MS. The results show that the ligand-drug conjugates (ADCs) of the present application had little effect on the free steroids and endogenous corticosterone as well as plasma P1NP levels, and the ligand-drug conjugates (ADCs) of the present application had biological safety.

### Example 9

### 9.1 Inhibitory Assay for IFNα Production in Human Peripheral Blood Monocytes

### Objective

The inhibitory effect of the ligand-drug conjugates (ADCs) of the present application on the production of IFNα by plasmacytoid dendritic cells. The plasmacytoid dendritic cells were treated in vitro with the ligand-drug conjugates at different concentrations, and IFNα produced by the plasmacytoid dendritic cells were quantitatively detected after a certain time. The ligand-drug conjugates were evaluated for in-vitro activity according to IC50.

### Test Method

1. Human peripheral blood monocytes were resuspended in RPMI complete medium, seeded at 0.5-1×10⁶ cells/well into a 96-well plate, to which the serially diluted ligand-drug conjugates of the present application were added. A stimulator CpG-A or R848 or a systemic lupus erythematosus SLE immune complex (Sm/RNP antigen and anti-RNP antibody were mixed at a certain ratio) with a fixed concentration was added into the system, and incubated at 37° C. overnight. The supernatant was collected, and the level of IFNα in the supernatant was detected using a kit. Dose-response data were fitted to an S-shaped curve using non-linear regression, and IC₅₀ values were calculated.

2. Plasmacytoid dendritic cells were isolated from the human peripheral blood monocytes using a plasmacytoid dendritic cell isolation kit (Miltenyi), suspended in RPMI complete medium, seeded at 0.5-2×10⁵ cells/well into a 96-well plate, to which the serially diluted ligand-drug conjugates of the present application were added. A stimulator CpG-A or R848 or a SLE immune complex (Sm/RNP antigen and anti-RNP antibody were mixed at a certain ratio) with a fixed concentration was added into the system, and incubated at 37° C. overnight. The supernatant was collected, and the level of IFNα in the supernatant was detected using a kit. Dose-response data were fitted to an S-shaped curve using non-linear regression, and IC₅₀ values were calculated.

The results show that the ligand-drug conjugates of the present application could affect the ability of plasmacytoid dendritic cells to produce IFNα. The ligand-drug conjugates of the present application can inhibit inflammation, and can be used for preventing and/or treating diseases and/or conditions such as inflammation.

### 9.2 Type I Interferon-Induced Response Element Signal Detection

HEK293 cells were constructed and transferred into pHTS-ISRE fluorescein reporter gene plasmid, and a reporter system of type I interferon response signals were constructed. The cells were cultured in a DMEM medium containing FBS and Geneticin with certain concentrations, and the serially-diluted ligand-drug conjugate and type I interferon IFN or recombinant type I interferon with certain concentrations induced by plasmacytoid dendritic cells were added for incubation. The cells were lysed, fluorescein intensity was determined, dose-response data were fitted to an S-shaped curve using non-linear regression, and IC₅₀ values were calculated. The results show that the ligand-drug conjugates of the present application could influence type I interferon-induced response element signals. The ligand-drug conjugates of the present application can inhibit inflammation, and can be used for preventing and/or treating diseases and/or conditions such as inflammation.

### 9.3 Cytokine Release Induced by CpG-A and the Like and Downstream Signal Molecule Detection

Primary human peripheral blood monocytes (PBMCs) were washed in 50 mL PBS, resuspended in FBS (fetal bovine serum) with 5% DMSO, divided in equal and cryopreserved in liquid nitrogen until use. The PBMCs were thawed, resuspended in cell culture medium (e.g., RPMI cell culture medium) with 2% FBS and 1% penicillin streptomycin, and seeded onto a 96-well plate. The cells were then incubated with different concentrations of the ligand-drug conjugates at 37° C. with 5% CO₂. The cells were treated with CpG-A or other stimulants at a certain concentration for a certain time. The plate was then spun at 1000 rpm for 5 min and 100 μL of the supernatant culture medium was transferred directly into another 96-well plate, and the levels of cytokines such as IL-6 and IL-10 were analyzed.

The ligand-drug conjugate with a certain concentration was added into human peripheral blood monocytes for culturing. Then type I interferon IFN or recombinant type I interferon with certain concentrations induced by plasmacytoid dendritic cells was added for incubation. The lysed cells were subjected to electrophoresis and western blot detection, and STAT1 phosphorylation levels were determined using anti-human STAT1 pTY701 antibody.

The results show that the ligand-drug conjugates (ADCs) of the present application had the ability to affect the release of cytokines from PBMC cells, and the ability to phosphorylate STAT1.

### 9.4 Evaluation of Drug Efficacy in Plasmacytoid Dendritic Cell Xenograft Mouse Model

### Objective

The ligand-drug conjugates (ADCs) of the present application act on a human plasmacytoid dendritic cell xenograft mouse model, and the in-vivo drug effect of the ligand-drug conjugates is evaluated by means of immunohistochemistry, gene transcription analysis and the like.

### Test Method

### 1. Imiquimod Induction Model

The backs of 4-8 week-old severely-immunodeficient mice (CB17/Icr-Prkdcscid/IcrIcoCrl, Charles River) were shaved. The 5% imiquimod cream was applied to the backs of the mice, and after 12 h, a second application was made. The mice were then injected intraperitoneally with a certain concentration of the ligand-drug conjugates of the present application or the control ligand-drug conjugate. After 12 h, 1-10×10⁵ human plasmacytoid dendritic cells were injected into the tail vein of the mice. After 12 h of incubation, the mice were euthanized and dorsal skin samples were collected for testing.

### 2. Bleomycin Induction Model

The backs of 4-8 week-old severely-immunodeficient mice (CB17/Icr-Prkdcscid/IcrIcoCrl, Charles River) were shaved. Bleomycin with a certain concentration was injected subcutaneously into a single site on the backs of the mice for three weeks every two days. 1-10×10⁵ human plasmacytoid dendritic cells were injected into the tail vein of the mice on Days 0, 7 and 14 of the first injection of bleomycin. The mice were injected intraperitoneally with the ligand-drug conjugates of the present application or the control ligand-drug conjugate with a certain concentration 24 h before the first injection of bleomycin, and the injection was performed once every 5 days.

### 3. Detection Method and Index

(1) In the kit, mouse skin cell RNA was extracted and subjected to reverse transcription to obtain cDNA, and the influence of the ligand-drug conjugate on the transcription of the type I IFN signaling pathway response gene was determined by utilizing Real-time PCR and taking a mouse sample treated with a control ligand-drug conjugate as a control.

(2) Mouse skin samples were fixed with formalin and embedded in paraffin, which was then sectioned at 5 μM for hematoxylin-eosin staining. The degree of fibrosis of the skin samples was identified by Masson staining. Immunohistochemical analysis was performed with the pSTAT1 Tyr701 antibody.

(3) After being processed, the mouse skin samples were analyzed by FACS, and the proportion of plasmacytoid dendritic cells in the mouse skin samples was determined according to the cell surface markers.

(4) The collagen content of the mouse skin samples was measured by a collagen analysis method after being processed.

The results show that the ligand-drug conjugates of the present application could affect (1) the ability of the type I IFN signaling pathway to respond to gene transcription, (2) the degree of fibrosis in a skin sample, (3) the ability of plasmacytoid dendritic cells to proliferate and/or (4) the collagen content of skin samples. The ligand-drug conjugate of the present application can inhibit inflammation in vivo, and can be used for preventing and/or treating diseases and/or conditions such as inflammation.

### Example 10

10.1 Cytokine Release Experiment for CD40L and LPS-Induced Dendritic Cells Derived from Human Peripheral Blood Monocytes

Fresh human peripheral blood monocytes (PBMCs) were prepared. Human monocytes were isolated using a human monocyte (CD14+) isolation kit. After washing, the cells were resuspended, added with human IL-4 (80 ng/mL) and human GM-CSF (100 ng/mL) and placed in an incubator and cultured for 5 days.

The cells were harvested, washed, added into a 96-well flat-bottom plate at 10⁵ cells/well, and stimulated with 0.1 ng/mL LPS for 2 h, and then the supernatant was washed off. A positive compound with a certain concentration was added and incubated for 2 h. 1 μg/mL Mega CD40L and 0.2 ng/mL LPS were added and incubated in an incubator at 37° C. with 5% CO₂ for 20 h.

The cell supernatant was collected by centrifugation, and the TNFα and IL-6 levels in the supernatant were detected using a kit. Dose-response data were fitted to an S-shaped curve using non-linear regression, and IC50 values were calculated.

The results show that the ligand-drug conjugates of the present application could inhibit cytokine release from dendritic cells derived from human peripheral blood monocyte. The ligand-drug conjugates of the present application can inhibit inflammation, and can be used for preventing and/or treating diseases and/or conditions such as inflammation.

### 10.2 Evaluation of Drug Efficacy in LPS-Induced Acute Inflammation Model

8-10 week-old CD40 humanized female mice were randomly grouped and injected intraperitoneally with a concentration of the ligand-drug conjugates of the present application or the control ligand-drug conjugate. After 2-16 h, the mice were injected intraperitoneally with LPS with a certain concentration. After 6 h, 100 μL of blood sample was collected, and the TNFα, IL-6 and IL-10 contents were measured using a kit. After another 18 h, the mice were euthanized, spleens were collected for single cell treatment, and the cells were stained with antibodies to determine the proportion of activated dendritic cells.

The results show that the ligand-drug conjugates of the present application could inhibit the release of cytokines in an LPS-induced acute inflammation mouse model and reduce the proportion of activated dendritic cells in the spleen. The ligand-drug conjugates of the present application can inhibit inflammation, and can be used for preventing and/or treating diseases and/or conditions such as inflammation.

### Example 11

### Objective

To investigate the pharmacokinetic properties of the drug in monkeys and observe the toxic manifestation of the animals after a single intravenous drip of the ligand-drug conjugates (ADCs) of the present application in monkeys.

### Test Method

Pharmacokinetics: after the single intravenous drip of the ligand-drug conjugates (ADCs) of the present application at difference doses in monkeys, blood samples were collected at a plurality of continuous time points, and the concentration of the drugs in the blood was detected by a specific detection method.

Toxicity study: after the single intravenous drip of the ligand-drug conjugates (ADCs) of the present application at difference doses in monkeys, the tolerance of animals and drug-related toxicity toxic manifestation were investigated in multiple aspects such as clinical observation, body weight and food intake, hematology, blood biochemistry, urine and gross anatomy.

The results show that: (1) after the ligand-drug conjugates (ADCs) of the present application were subjected to single intravenous drip on a monkey, the concentration of free drugs was very low, and the pharmacokinetic properties of a total antibody and the ligand-drug conjugates (ADCs) of the present application were similar, so that the ligand-drug conjugates (ADCs) of the present application were slowly released in the monkey, the coupling mode was stable, and the clinically planned administration frequency could be supported; and (2) after single intravenous drip of the ligand-drug conjugates (ADCs) of the present application in monkeys, animals had good tolerance and did not show serious or intolerable drug-related toxicity, which indicated that the ligand-drug conjugates (ADCs) of the present application were controllable in safety and could support further clinical application.

The foregoing detailed description is provided by way of illustration and example, and is not intended to limit the scope of the appended claims. Various modifications of the embodiments currently enumerated in the present application will be apparent to those of ordinary skill in the art and are intended to be within the scope of the appended claims and their equivalents.

What is claimed is:

1. A compound or a tautomer, a mesomer, a racemate, an enantiomer or a diastereoisomer thereof, or a mixture thereof, or a pharmaceutically acceptable salt thereof, wherein the compound comprises a structure shown as formula (II): 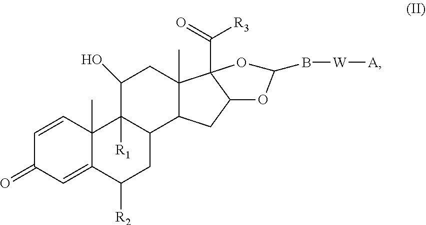
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and each Y is independently selected from the group consisting of: hydrogen, protium, deuterium, tritium, optionally substituted —OH, optionally substituted —CH₂—OH, optionally substituted —SH, optionally substituted —PH₂, optionally substituted —P(═O)H₂, and optionally substituted —NH₂.

2. A compound or a tautomer, a mesomer, a racemate, an enantiomer or a diastereoisomer thereof, or a mixture thereof, or a pharmaceutically acceptable salt thereof, wherein the compound comprises a structure shown as formula (II-a1) or formula (II-a2): 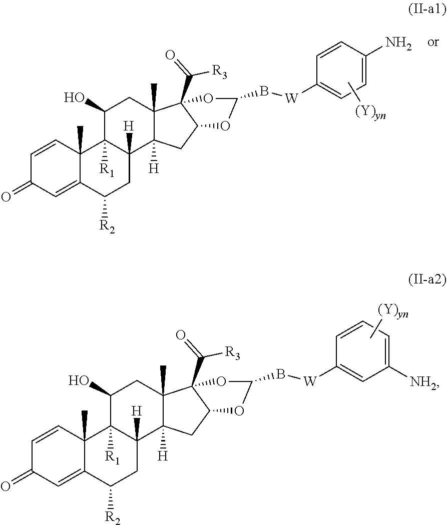
wherein R₁, R₂, R₃ and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4.

3. The compound or the tautomer, the mesomer, the racemate, the enantiomer or the diastereoisomer thereof, or the mixture thereof, or the pharmaceutically acceptable salt thereof according to claim 1 or 2, wherein
R₁ is selected from the group consisting of: hydrogen, protium, deuterium, tritium, halogen, and optionally substituted C₁-C₆ alkyl.

4. The compound or the tautomer, the mesomer, the racemate, the enantiomer or the diastereoisomer thereof, or the mixture thereof, or the pharmaceutically acceptable salt thereof according to claim 3, wherein
R₁ is selected from the group consisting of: hydrogen, F, Cl, Br, and optionally substituted methyl.

5. The compound or the tautomer, the mesomer, the racemate, the enantiomer or the diastereoisomer thereof, or the mixture thereof, or the pharmaceutically acceptable salt thereof according to claim 1 or 2, wherein
R₂ is selected from the group consisting of: hydrogen, protium, deuterium, tritium, halogen, and optionally substituted C₁-C₆ alkyl.

6. The compound or the tautomer, the mesomer, the racemate, the enantiomer or the diastereoisomer thereof, or the mixture thereof, or the pharmaceutically acceptable salt thereof according to claim 5, wherein
R₂ is selected from the group consisting of: hydrogen, F, Cl, Br, and optionally substituted methyl.

7. The compound or the tautomer, the mesomer, the racemate, the enantiomer or the diastereoisomer thereof, or the mixture thereof, or the pharmaceutically acceptable salt thereof according to claim 1 or 2, wherein
R₃ is selected from —CH₂OH, —SCH₂F,

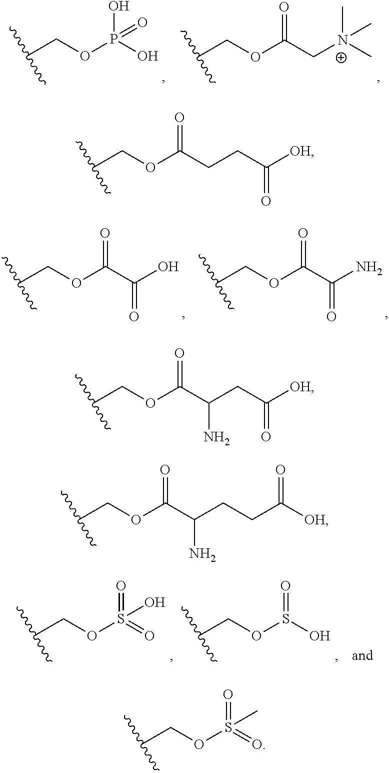

8. The compound or the tautomer, the mesomer, the racemate, the enantiomer or the diastereoisomer thereof, or the mixture thereof, or the pharmaceutically acceptable salt thereof according to any one of claims 1-7, wherein
B is selected from the group consisting of: optionally substituted pyrrolyl and optionally substituted thienyl.

9. The compound or the tautomer, the mesomer, the racemate, the enantiomer or the diastereoisomer thereof, or the mixture thereof, or the pharmaceutically acceptable salt thereof according to claim 8, wherein
B is selected from the group consisting of: optionally substituted 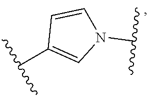 [SMILES: *c1ccn(*)c1]
optionally substituted 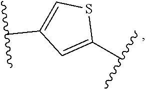 [SMILES: *c1csc(*)c1]
optionally substituted 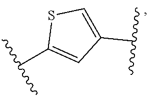 [SMILES: *c1csc(*)c1]
and optionally substituted

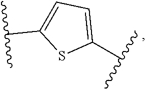 [SMILES: *c1ccc(*)s1]

10. The compound or the tautomer, the mesomer, the racemate, the enantiomer or the diastereoisomer thereof, or the mixture thereof, or the pharmaceutically acceptable salt thereof according to any one of claims 1-9, wherein
B is selected from the group consisting of optionally substituted 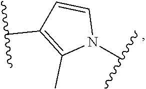 [SMILES: *c1ccn(*)c1C]
optionally substituted 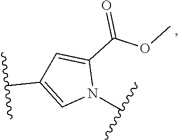 [SMILES: *c1cc(C(=O)OC)n(*)c1]
optionally substituted 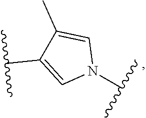 [SMILES: *c1cn(*)cc1C]
optionally substituted 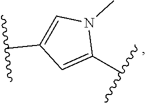 [SMILES: *c1cc(*)n(C)c1]
optionally substituted 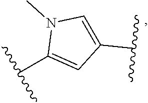 [SMILES: *c1cc(*)n(C)c1]
optionally substituted 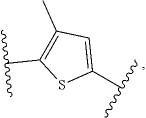 [SMILES: *c1cc(C)c(*)s1]
optionally substituted 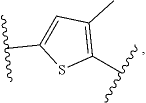 [SMILES: *c1cc(C)c(*)s1]
optionally substituted 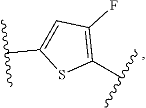 [SMILES: *c1cc(F)c(*)s1]
optionally substituted 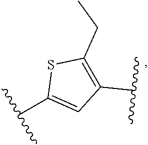 [SMILES: *c1cc(*)c(CC)s1]
optionally substituted 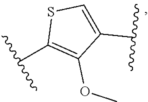 [SMILES: *c1csc(*)c1OC]
optionally substituted F 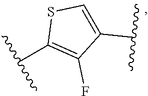 [SMILES: *c1csc(*)c1F]
optionally substituted 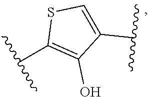 [SMILES: *c1csc(*)c1O]
optionally substituted 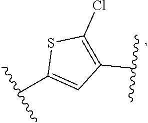 [SMILES: *c1cc(*)c(Cl)s1]
optionally substituted 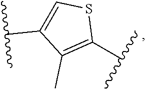 [SMILES: *c1csc(*)c1C]
and optionally substituted

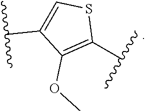 [SMILES: *c1csc(*)c1OC]

11. The compound or the tautomer, the mesomer, the racemate, the enantiomer or the diastereoisomer thereof, or the mixture thereof, or the pharmaceutically acceptable salt thereof according to any one of claims 1-10, wherein
W is absent, or W is selected from the group consisting of: —S(═O)—, —S(═O)₂—, —O—, optionally substituted —NH—, optionally substituted C₁-C₆ alkylene, optionally substituted C₂-C₆ alkenylene, and optionally substituted C₂-C₆ alkynylene, wherein, when W comprises a methylene unit, the methylene unit of W is each independently unreplaced, or the methylene unit of W is each independently replaced by a group selected from the group consisting of: —O—, —S—, optionally substituted —NH—, and optionally substituted alcylene.

12. The compound or the tautomer, the mesomer, the racemate, the enantiomer or the diastereoisomer thereof, or the mixture thereof, or the pharmaceutically acceptable salt thereof according to any one of claims 1-11, wherein
W is absent, or W is selected from the group consisting of: —S(═O)—, —S(═O)₂—, —O—, —S—, optionally substituted —NH—, optionally substituted —CH═CH—, —C≡C—, optionally substituted C₁-C₆ alkylene, optionally substituted —OCH₂—, optionally substituted —CH₂O—, optionally substituted —SCH₂—, optionally substituted —CH₂S—, —CO—, optionally substituted —NHC(═O)—, optionally substituted —COCH₂—, optionally substituted —CH₂NH—, optionally substituted —NHCH(CH₃)—, optionally substituted —NHCH₂—, optionally substituted —CH(CH₃)—, optionally substituted 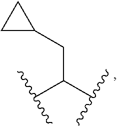 [SMILES: *C(*)CC1CC1]
optionally substituted 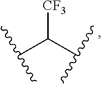 [SMILES: *C(*)C(F)(F)F]
optionally substituted 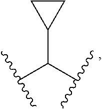 [SMILES: *C(*)C1CC1]
optionally substituted 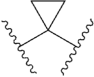 [SMILES: *C1(*)CC1]
and optionally substituted

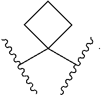 [SMILES: *C1(*)CCC1]

13. The compound or the tautomer, the mesomer, the racemate, the enantiomer or the diastereoisomer thereof, or the mixture thereof, or the pharmaceutically acceptable salt thereof according to any one of claims 1-12, wherein
W is absent or W is selected from the group consisting of the following structures that are optionally substituted:

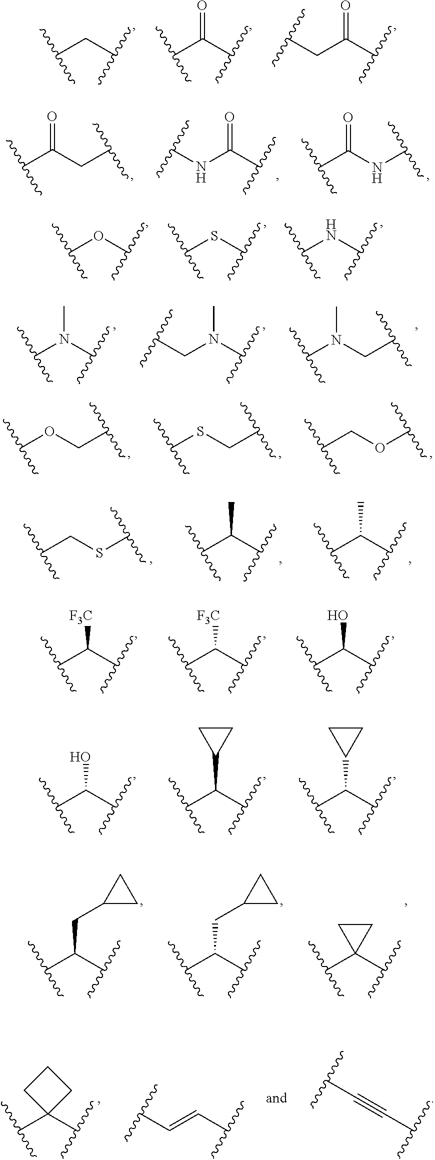

14. The compound or the tautomer, the mesomer, the racemate, the enantiomer or the diastereoisomer thereof, or the mixture thereof, or the pharmaceutically acceptable salt thereof according to any one of claims 1-13, wherein
W is selected from the group consisting of the following structures that are optionally substituted: optionally substituted —CH₂—, optionally substituted —CH(CH₃)—, and optionally substituted

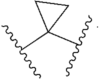 [SMILES: *C1(*)CC1]

15. The compound or the tautomer, the mesomer, the racemate, the enantiomer or the diastereoisomer thereof, or the mixture thereof, or the pharmaceutically acceptable salt thereof according to any one of claims 1-14, wherein
A is selected from the group consisting of: optionally substituted C₁-C₆ alkyl, optionally substituted C₃-C₁₀ alcyl, optionally substituted C₂-C₉ aliphatic heterocyclyl, optionally substituted C₆-C₁₀ aryl and optionally substituted C₁-C₉ heteroaryl.

16. The compound or the tautomer, the mesomer, the racemate, the enantiomer or the diastereoisomer thereof, or the mixture thereof, or the pharmaceutically acceptable salt thereof according to any one of claims 1-15, wherein
when A is optionally substituted phenyl, A is substituted with Rₐ₄, and Rₐ₄ is selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₄ comprises a methylene unit, the methylene unit of Rₐ₄ is each independently unreplaced, or the methylene unit of Rₐ₄ is each independently replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

17. The compound or the tautomer, the mesomer, the racemate, the enantiomer or the diastereoisomer thereof, or the mixture thereof, or the pharmaceutically acceptable salt thereof according to any one of claims 1-15, wherein
when A is optionally substituted C₅ heteroaryl, A is substituted with Rₐ₅, and Rₐ₅ is selected from the group consisting of: hydrogen, protium, deuterium, tritium, halogen, —NO₂, —CN, ═O, ═S, optionally substituted —S(═O)H, optionally substituted —S(═O)₂H, optionally substituted —C(═O)H, optionally substituted —OH, optionally substituted —SH, optionally substituted —PH₂, optionally substituted —P(═O)H₂, optionally substituted —NH₂, optionally substituted alkyl, optionally substituted alkenyl, optionally substituted alkynyl, optionally substituted alcyl, optionally substituted aliphatic heterocyclyl, optionally substituted aryl and optionally substituted heteroaryl; wherein, when Rₐ₅ comprises a methylene unit, the methylene unit of Rₐ₅ is each independently unreplaced, or the methylene unit of Rₐ₅ is each independently replaced by a group selected from the group consisting of: —S(═O)—, —S(═O)₂—, —O—, —S—, optionally substituted —PH—, optionally substituted —P(═O)H—, optionally substituted —NH—, optionally substituted alkylene, optionally substituted alkenylene, optionally substituted alkynylene, optionally substituted alcylene, optionally substituted aliphatic heterocyclylene, optionally substituted arylene and optionally substituted heteroarylene.

18. The compound or the tautomer, the mesomer, the racemate, the enantiomer or the diastereoisomer thereof, or the mixture thereof, or the pharmaceutically acceptable salt thereof according to claim 17, wherein
A is optionally substituted pyridinyl.

19. The compound or the tautomer, the mesomer, the racemate, the enantiomer or the diastereoisomer thereof, or the mixture thereof, or the pharmaceutically acceptable salt thereof according to any one of claims 1-18, wherein
A is selected from the group consisting of: optionally substituted —CH₂—Y, optionally substituted —(CH₂)₂—Y, optionally substituted —(CH₂)₃—Y, optionally substituted —CH₂—CH(—CH₃)—Y, optionally substituted —CH₂(—CH₂-cyclopropyl)-Y, optionally substituted cyclopropyl-Y, optionally substituted cyclobutyl-Y, optionally substituted cyclohexyl-Y, optionally substituted azetidinyl-Y, optionally substituted piperidinyl-Y, optionally substituted-phenyl-Y, optionally substituted-phenyl(—O—CH₃)—Y, optionally substituted-phenyl(—Cl)—Y, and optionally substituted-pyridinyl-Y.

20. The compound or the tautomer, the mesomer, the racemate, the enantiomer or the diastereoisomer thereof, or the mixture thereof, or the pharmaceutically acceptable salt thereof according to any one of claims 1-19, wherein
A is selected from the group consisting of: optionally substituted —CH₂—Y, optionally substituted —(CH₂)₂—Y, optionally substituted —(CH₂)₃—Y, optionally substituted —CH₂—CH(—CH₃)—Y,
optionally substituted —CH₂(—CH₂-cyclopropyl)-Y, optionally substituted 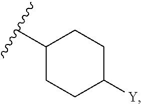
optionally substituted 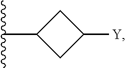
optionally substituted 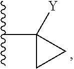
optionally substituted 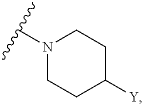
optionally substituted 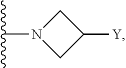
optionally substituted 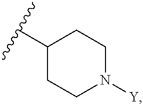
optionally substituted 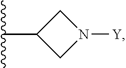
optionally substituted 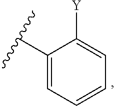
optionally substituted 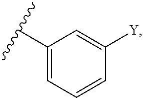
optionally substituted 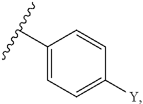
optionally substituted 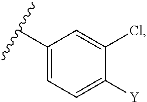
optionally substituted 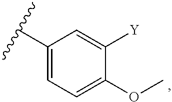
optionally substituted 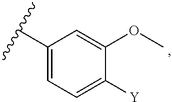
optionally substituted 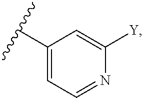
optionally substituted 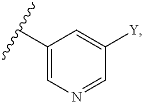
and optionally substituted

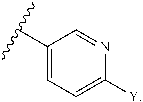

21. The compound or the tautomer, the mesomer, the racemate, the enantiomer or the diastereoisomer thereof, or the mixture thereof, or the pharmaceutically acceptable salt thereof according to any one of claims 1-20, wherein
A is selected from the group consisting of optionally substituted 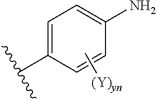
and optionally substituted 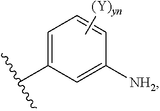
and yn is an integer from 0 to 4.

22. The compound or the tautomer, the mesomer, the racemate, the enantiomer or the diastereoisomer thereof, or the mixture thereof, or the pharmaceutically acceptable salt thereof according to any one of claims 1-21, wherein
each Y is independently selected from the group consisting of: hydrogen, optionally substituted —OH, optionally substituted —CH₂—OH, optionally substituted —SH, and optionally substituted —NH₂.

23. The compound or the tautomer, the mesomer, the racemate, the enantiomer or the diastereoisomer thereof, or the mixture thereof, or the pharmaceutically acceptable salt thereof according to any one of claims 1-21, wherein
each Y is independently selected from the group consisting of: optionally substituted hydroxy, optionally substituted alkylhydroxy, optionally substituted sulfhydryl, optionally substituted amino and optionally substituted C₁-C₆ alkyl.

24. The compound or the tautomer, the mesomer, the racemate, the enantiomer or the diastereoisomer thereof, or the mixture thereof, or the pharmaceutically acceptable salt thereof according to any one of claims 1-23, wherein the compound comprises a structure shown as formula (II-a1-1), (II-a1-2), (II-a1-3), (II-a2-1), (II-a2-2) or (II-a2-3): 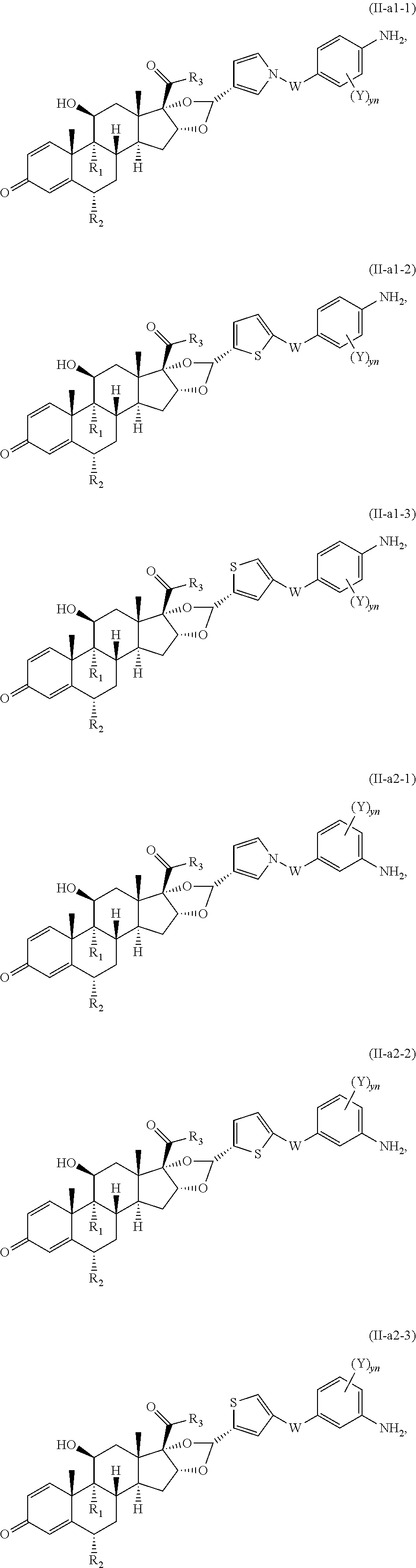
wherein
R₁ and R₂ are each independently selected from: hydrogen, protium, deuterium, tritium, halogen, and C₁-C₆ alkyl;
R₃ is selected from: —CH₂Cl, —CH₂SH, —CH₂OH, 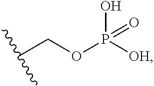 [SMILES: *COP(=O)(O)O]
—OCH₃, —OCH₂F, —OCH₂Cl, —OCH₂CN, —OCH₂CH₃, —SH, —SCH₂F, —SCH₂Cl, —SCH₂CF₃ and —SCH₂CN;
in the general formulas (II-a1-1) and (II-a2-1), Y is each independently selected from: hydrogen, halogen, hydroxy, sulfhydryl, amino, C₁-C₆ alkyl, and —OC₁-C₆ alkyl; in the general formulas (II-a1-2), (II-a1-3), (II-a2-2) and (II-a2-3), Y is each independently selected from: hydrogen, halogen, hydroxy, sulfhydryl, amino, C₁-C₆ alkyl, —OC₁-C₆ alkyl and —C₁-C₆ alkylhydroxy;
yn is selected from 0, 1 or 2;
W is selected from 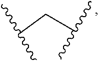 [SMILES: *C*]
—CH(CH₃)—and

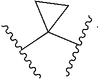 [SMILES: *C1(*)CC1]

25. A compound or a tautomer, a mesomer, a racemate, an enantiomer or a diastereoisomer thereof, or a mixture thereof, or a pharmaceutically acceptable salt thereof, wherein
the compound comprises a structure selected from the group consisting of:

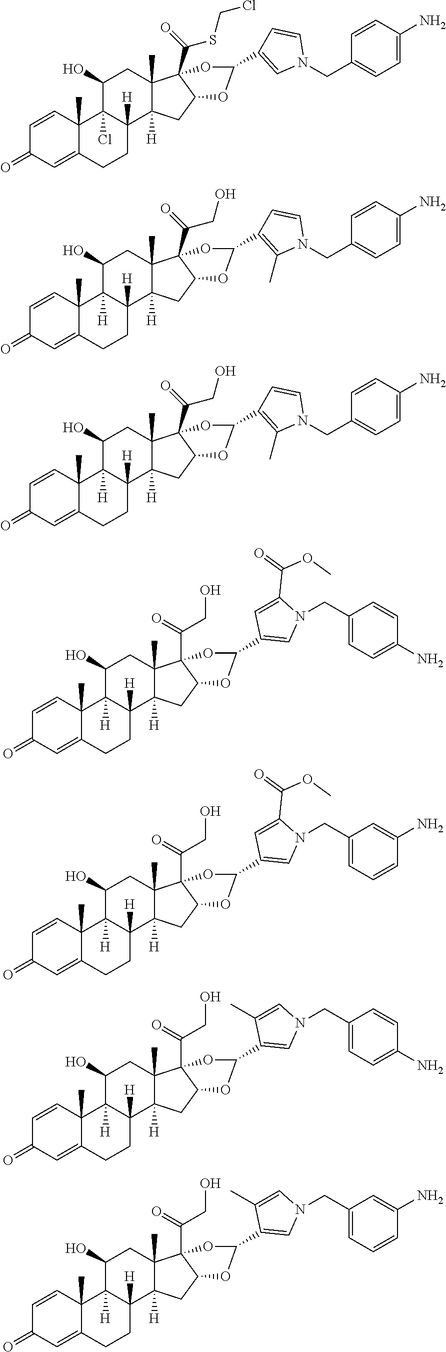

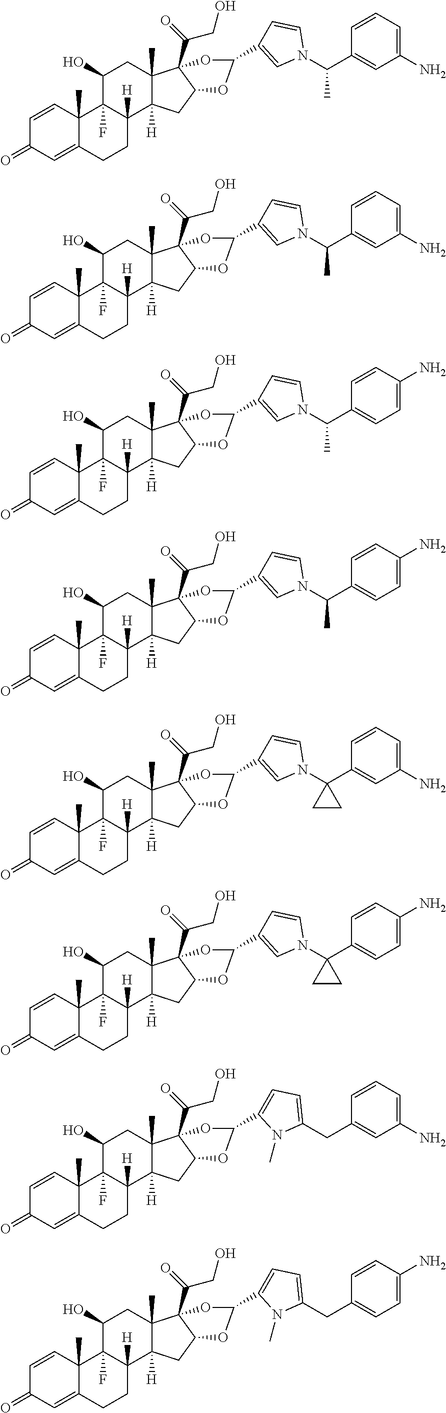

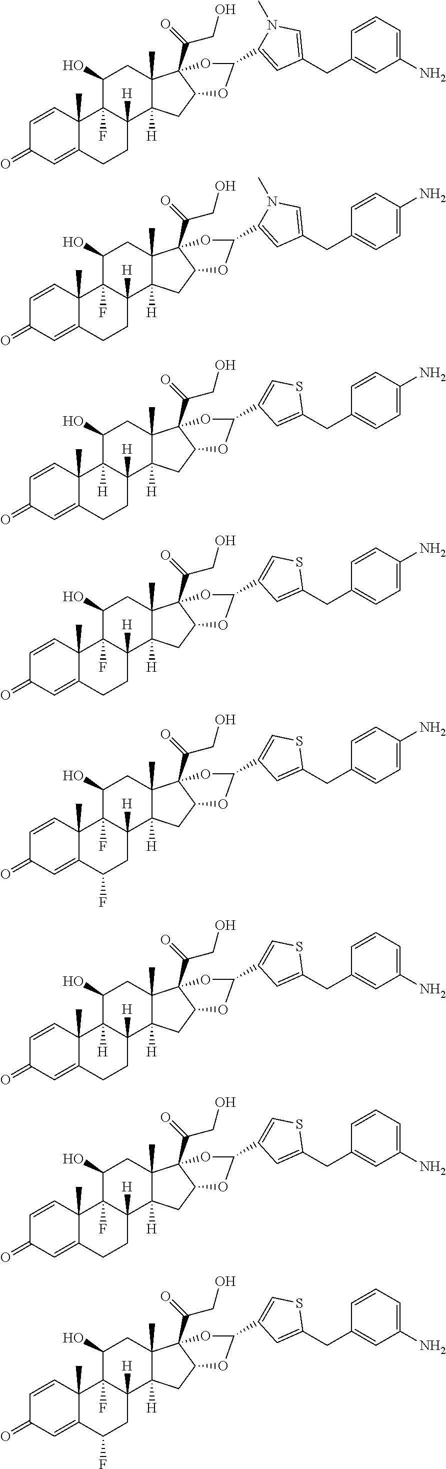

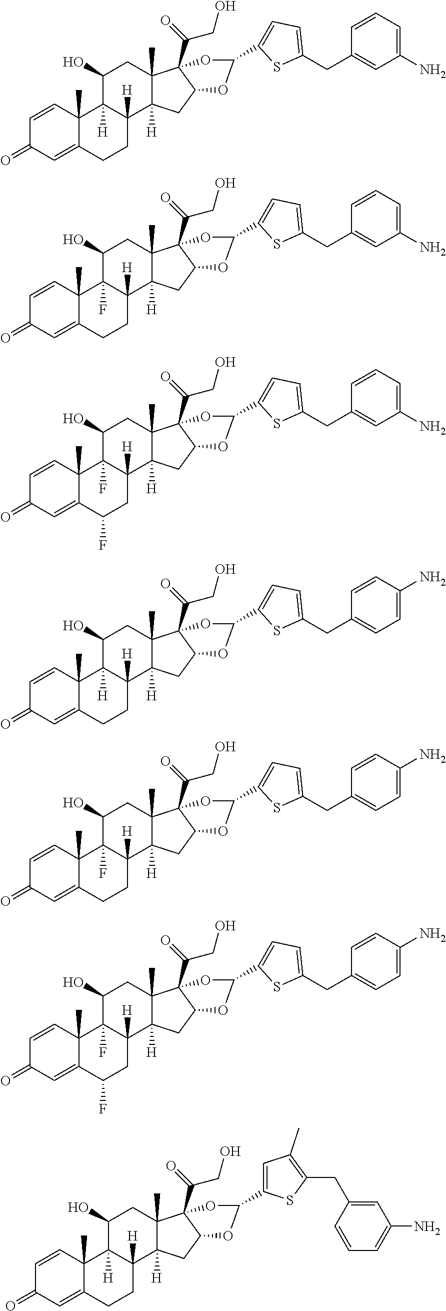

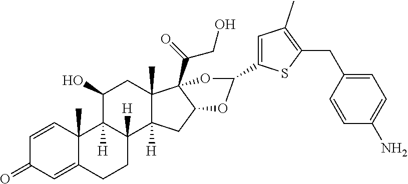

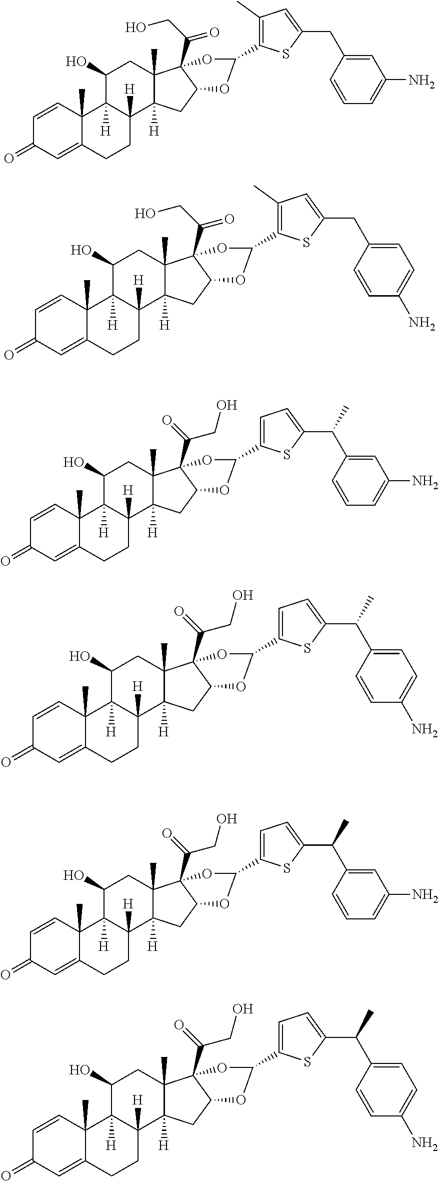

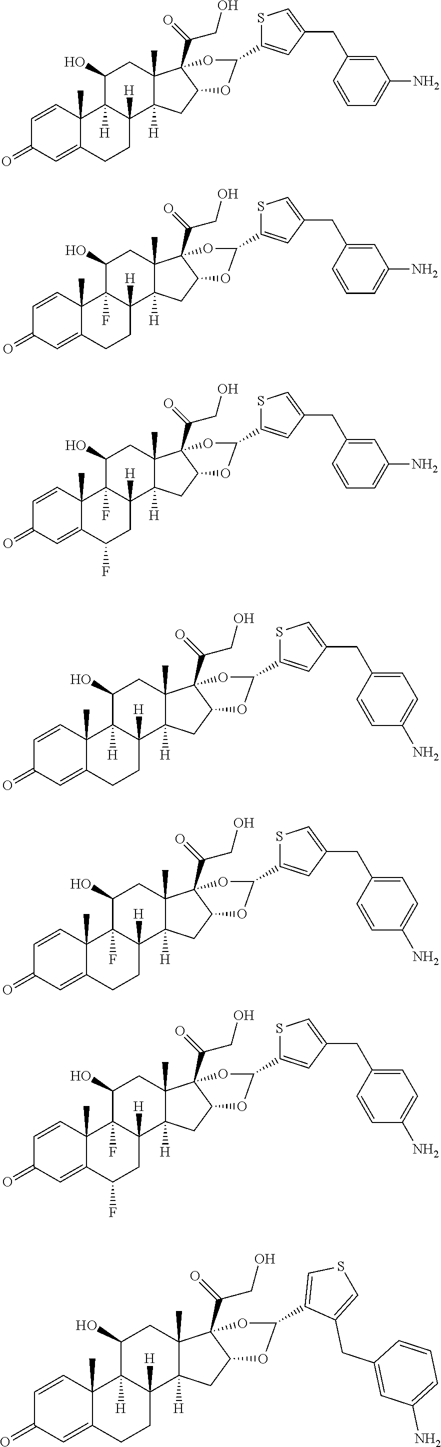

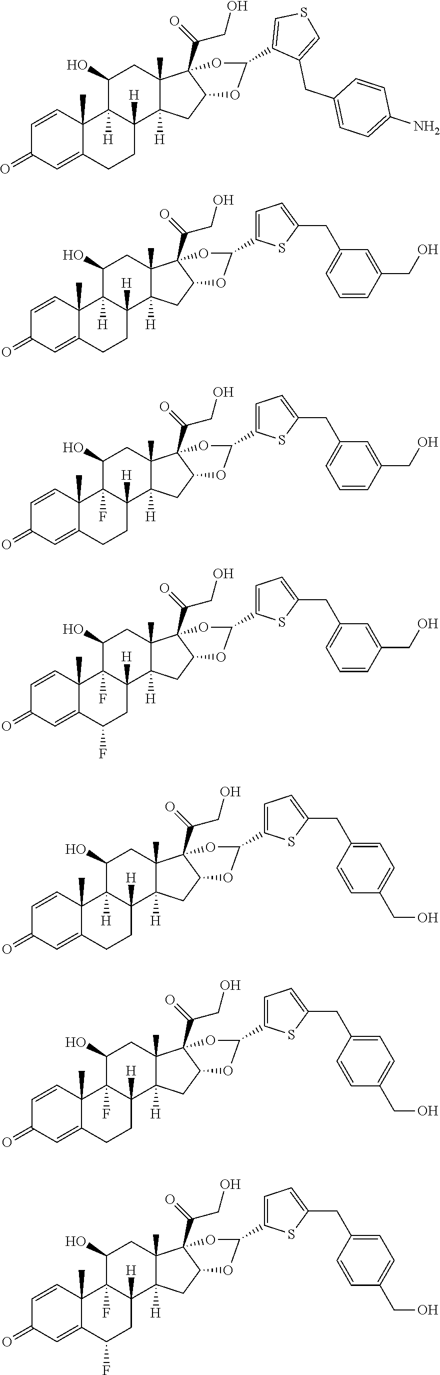

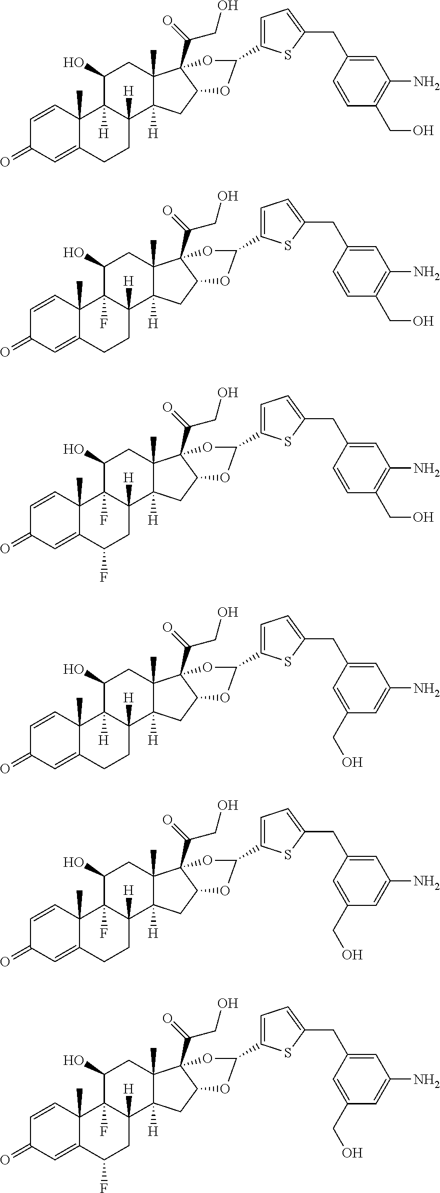

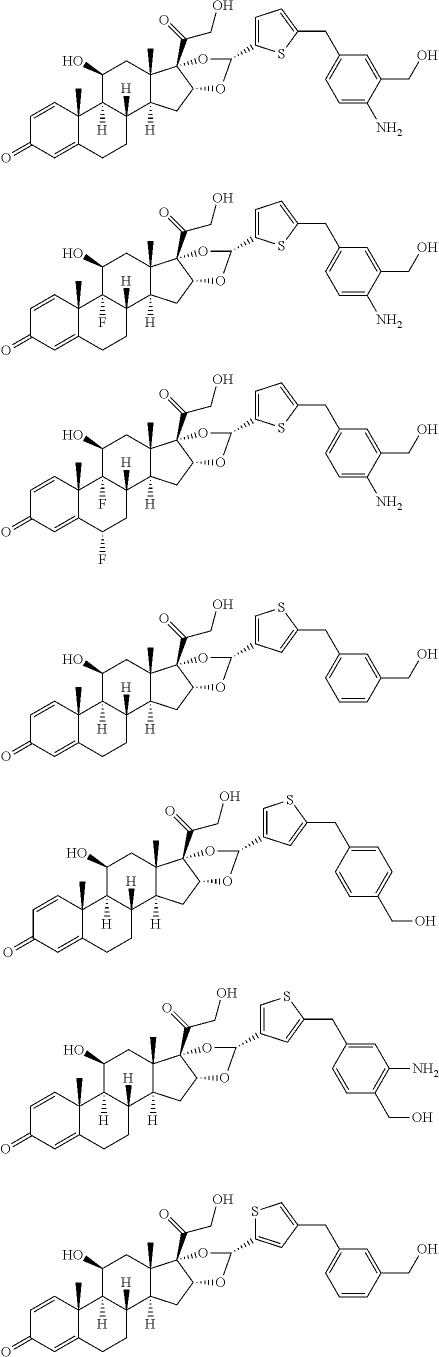

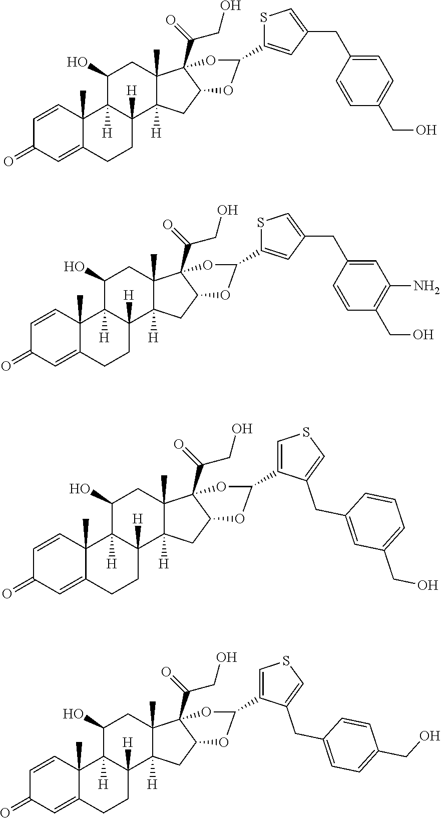

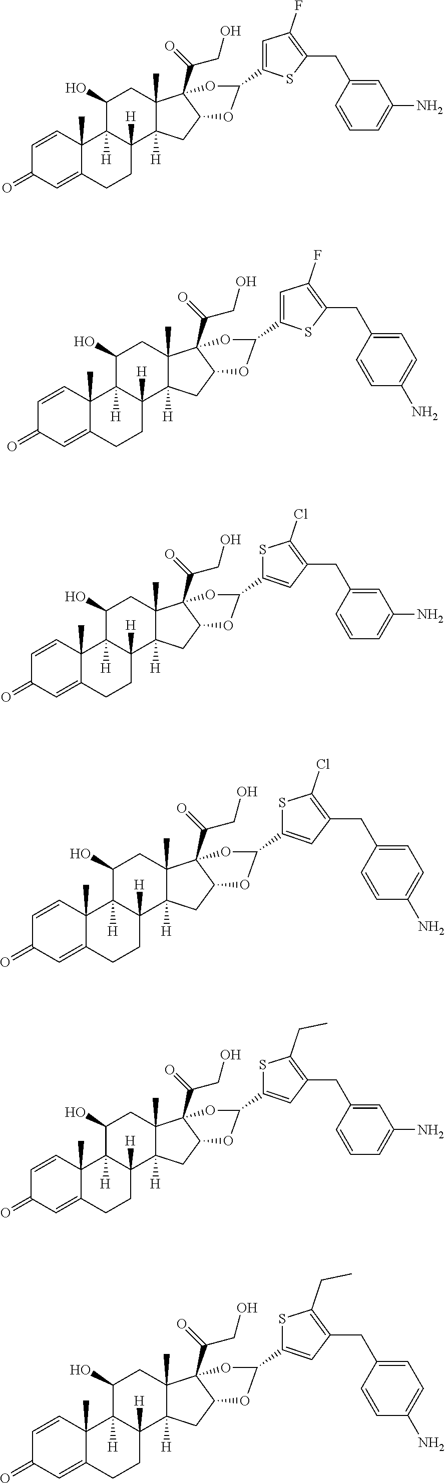

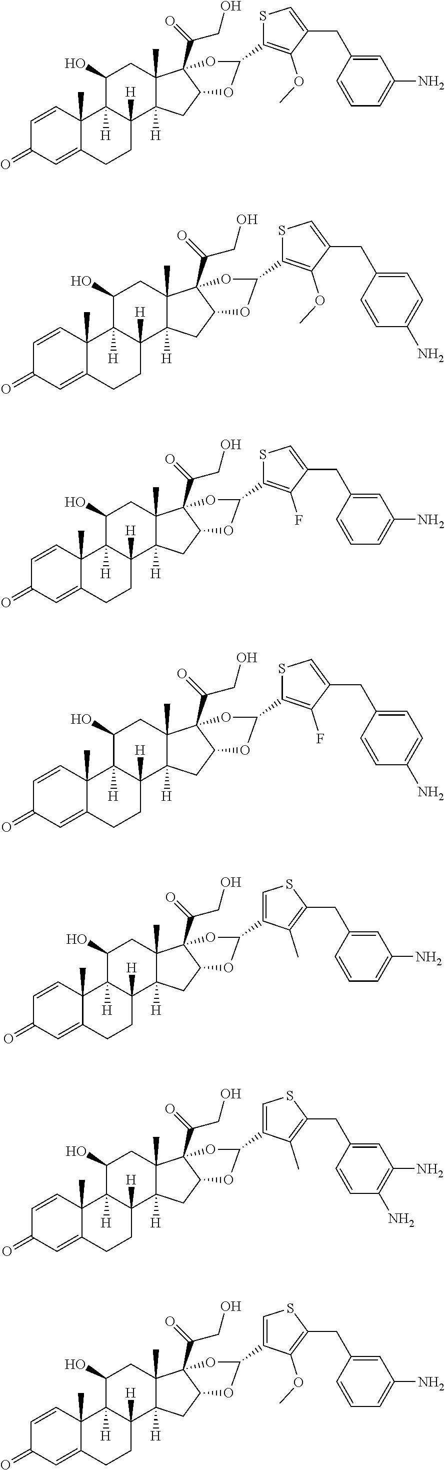

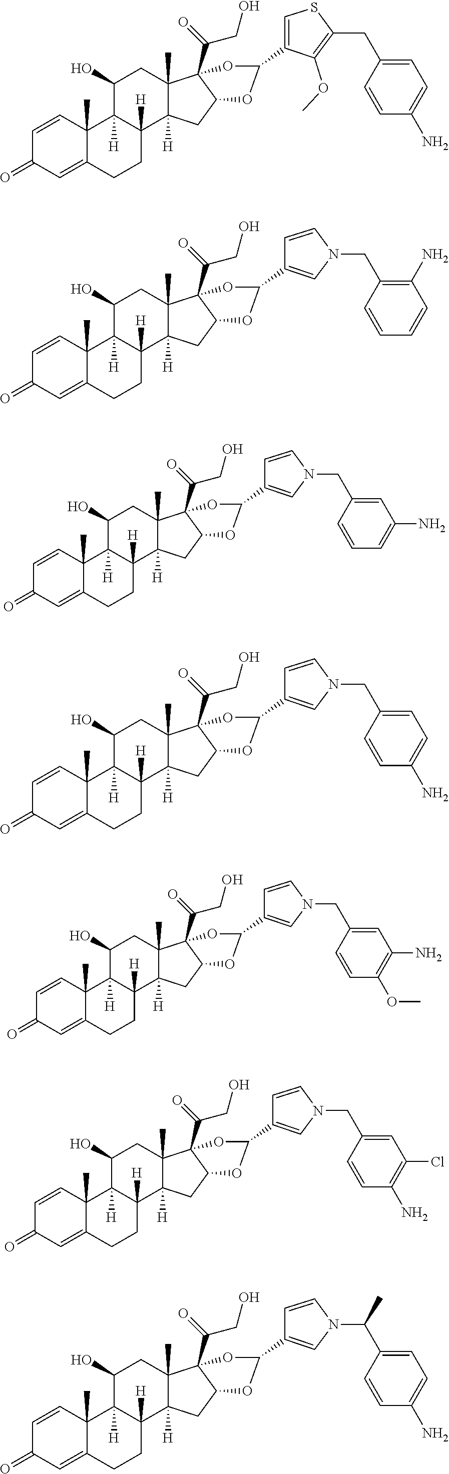

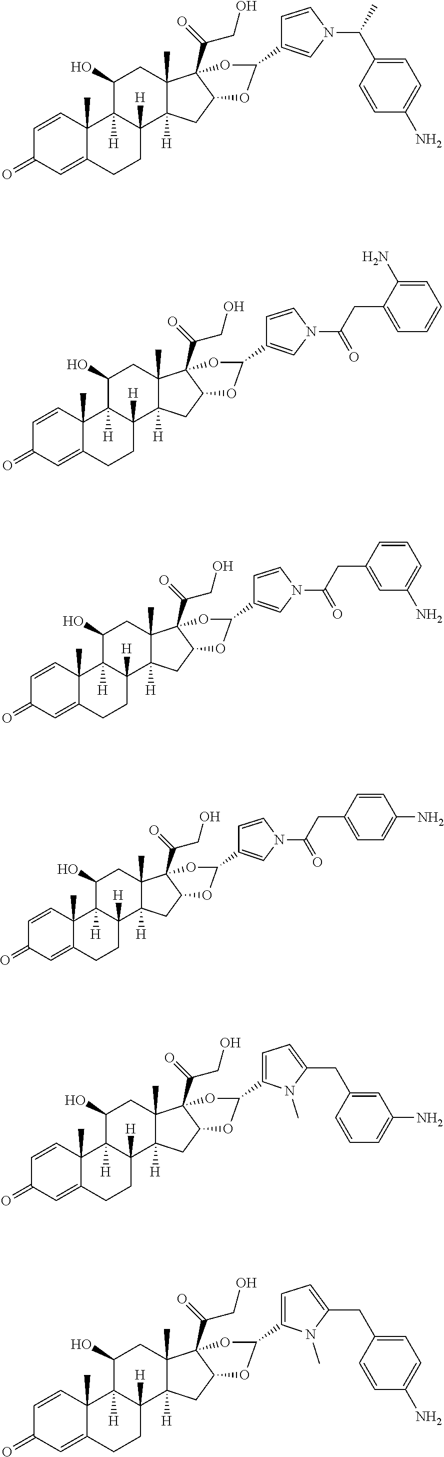

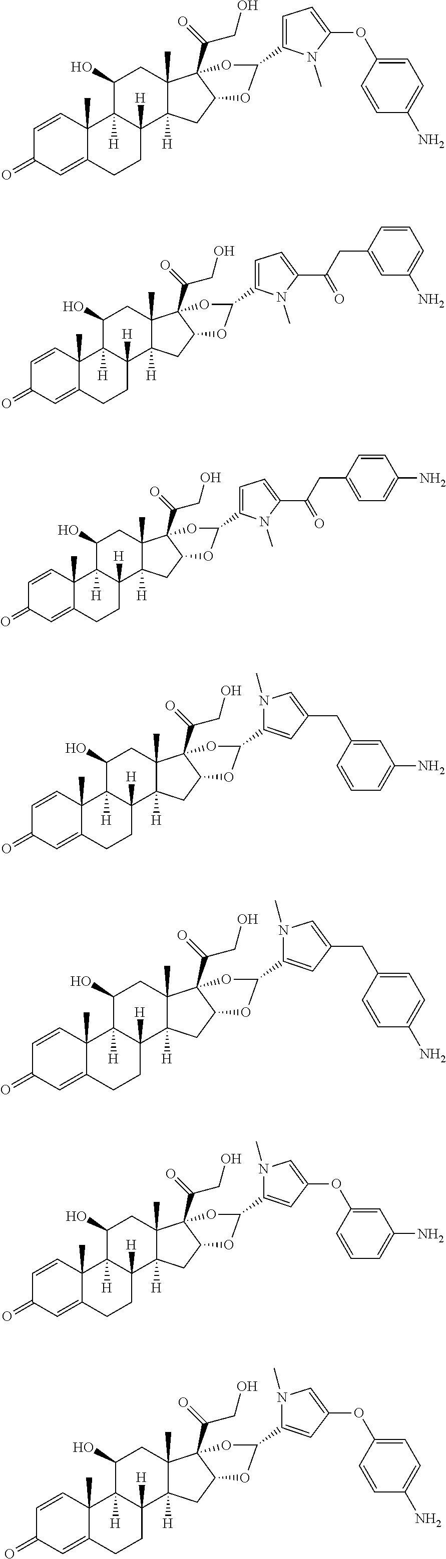

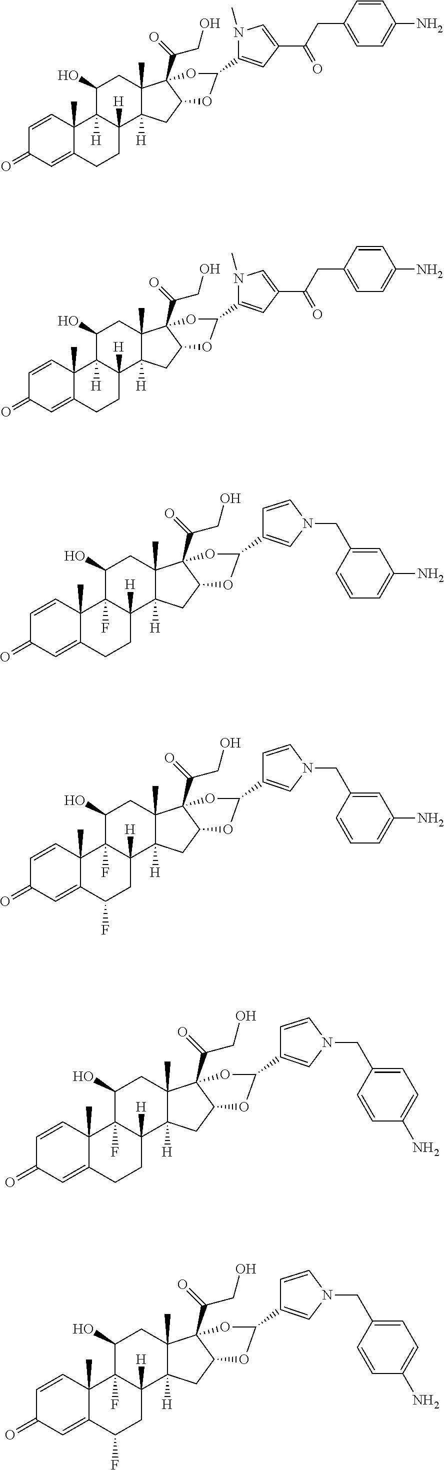

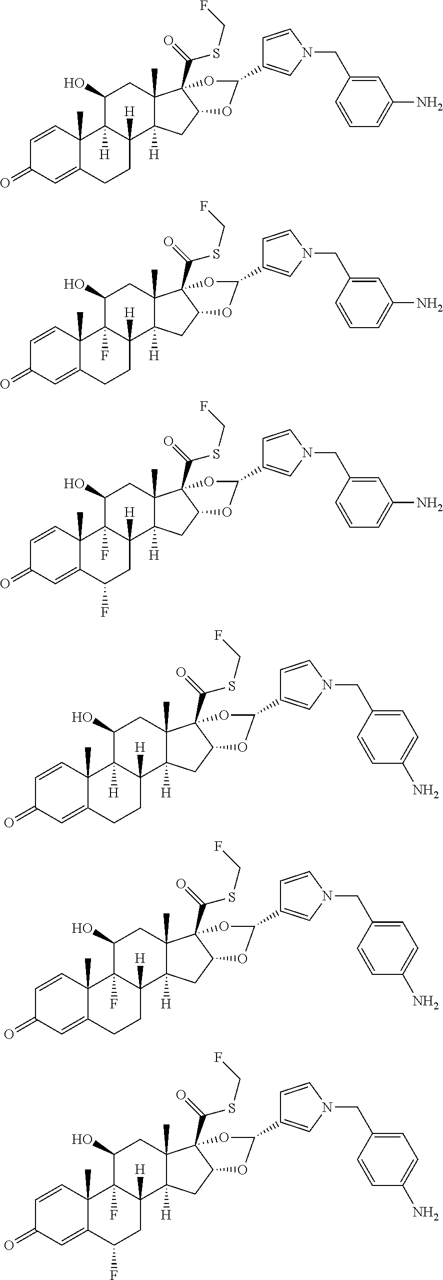

26. A compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

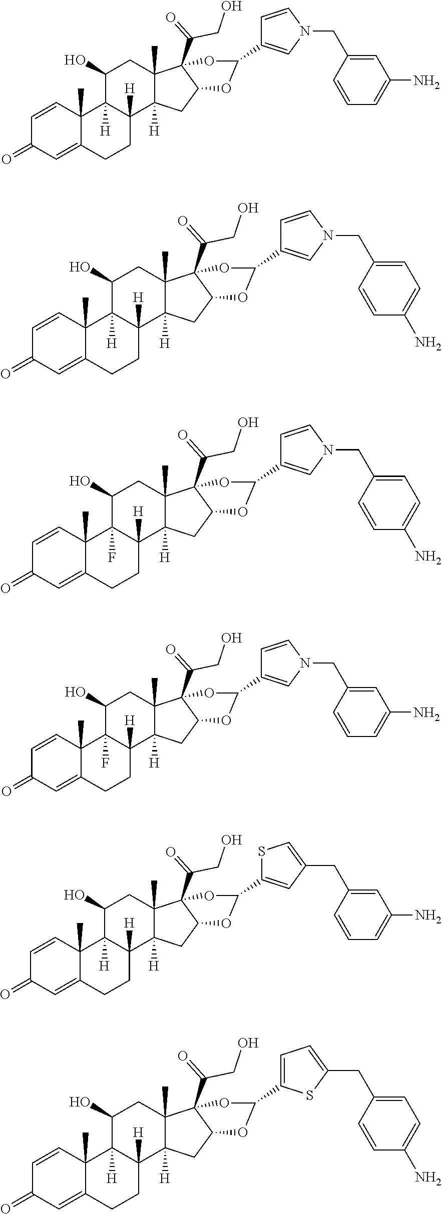

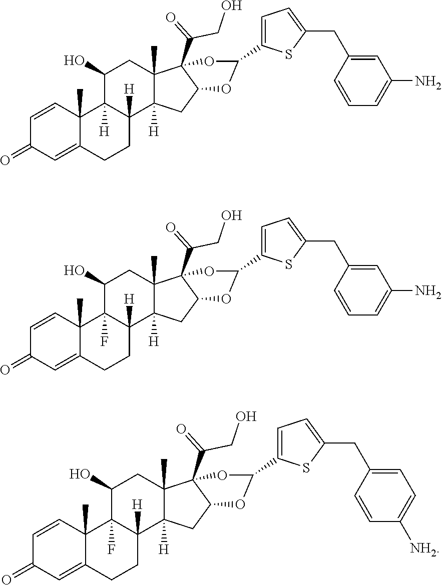

27. A conjugate, comprising the compound or the tautomer, the mesomer, the racemate, the enantiomer or the diastereoisomer thereof, or the mixture thereof, or the pharmaceutically acceptable salt thereof according to any one of claims 1-26.

28. The conjugate according to claim 27, comprising an antibody-drug conjugate.

29. A compound or a tautomer, a mesomer, a racemate, an enantiomer or a diastereoisomer thereof, or a mixture thereof, or a pharmaceutically acceptable salt thereof, wherein the compound comprises a structure shown as formula (II-A): 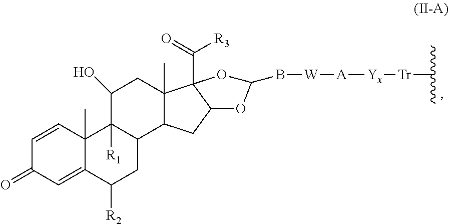
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; Tr is absent or is any group.

30. A compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-A), formula (II-a2-A), formula (II-a3-A) or formula (II-a4-A): 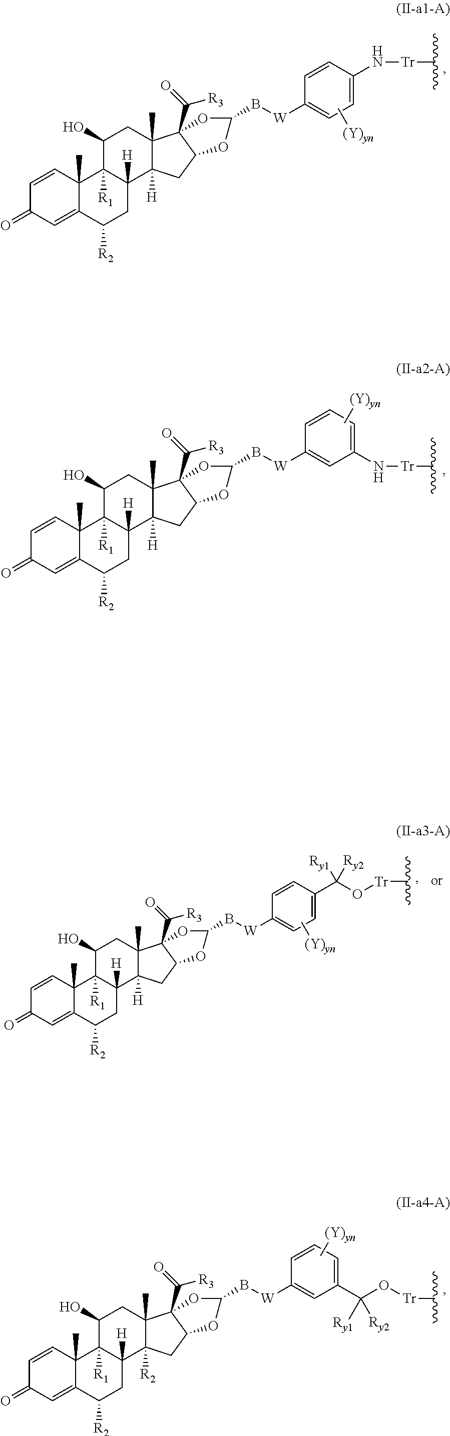
wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; Tr is absent or is any group.

31. A compound or a tautomer, a mesomer, a racemate, an enantiomer or a diastereoisomer thereof, or a mixture thereof, or a pharmaceutically acceptable salt thereof, wherein the compound comprises a structure shown as formula (II-B): 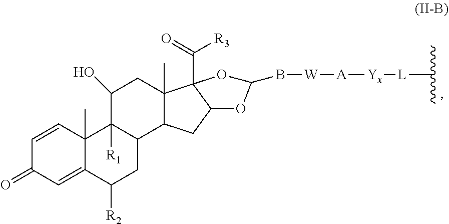
wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of —O—, optionally substituted —CH₂—O—, —S—, optionally substituted —PH—, optionally substituted —P(═O)H—, and optionally substituted —NH—; L is absent or is any group.

32. A compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-B), formula (II-a2-B), formula (II-a3-B) or formula (II-a4-B): 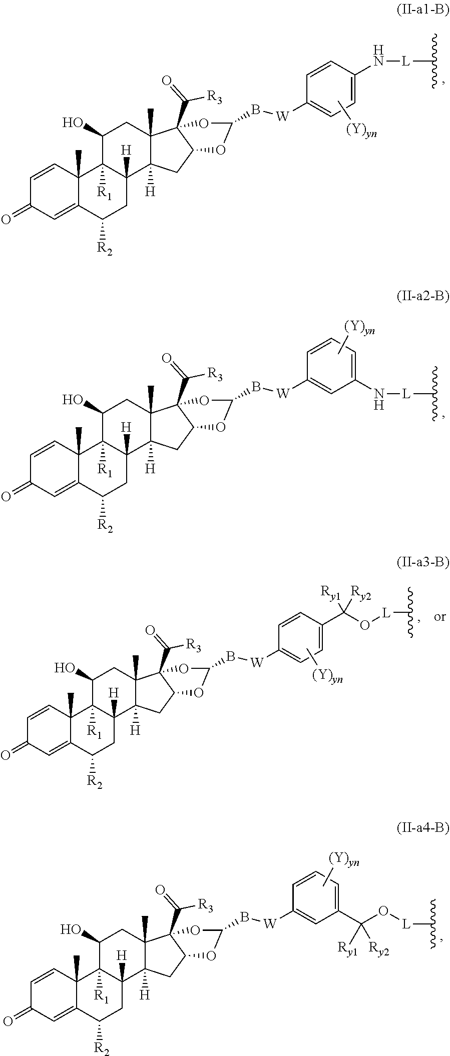
wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; L is absent or is any group.

33. A compound or a tautomer, a mesomer, a racemate, an enantiomer or a diastereoisomer thereof, or a mixture thereof, or a pharmaceutically acceptable salt thereof, wherein the compound comprises a structure shown as formula (II-C): 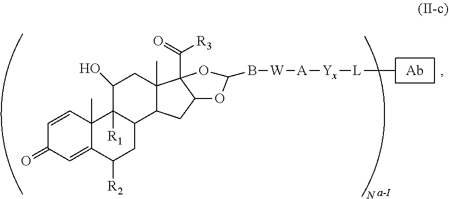
wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; L is absent or is any group; Ab is a ligand; N^{a-1} is a number equal to or greater than 0.

34. A compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-C), formula (II-a2-C), formula (II-a3-C or formula (II-a4-C): 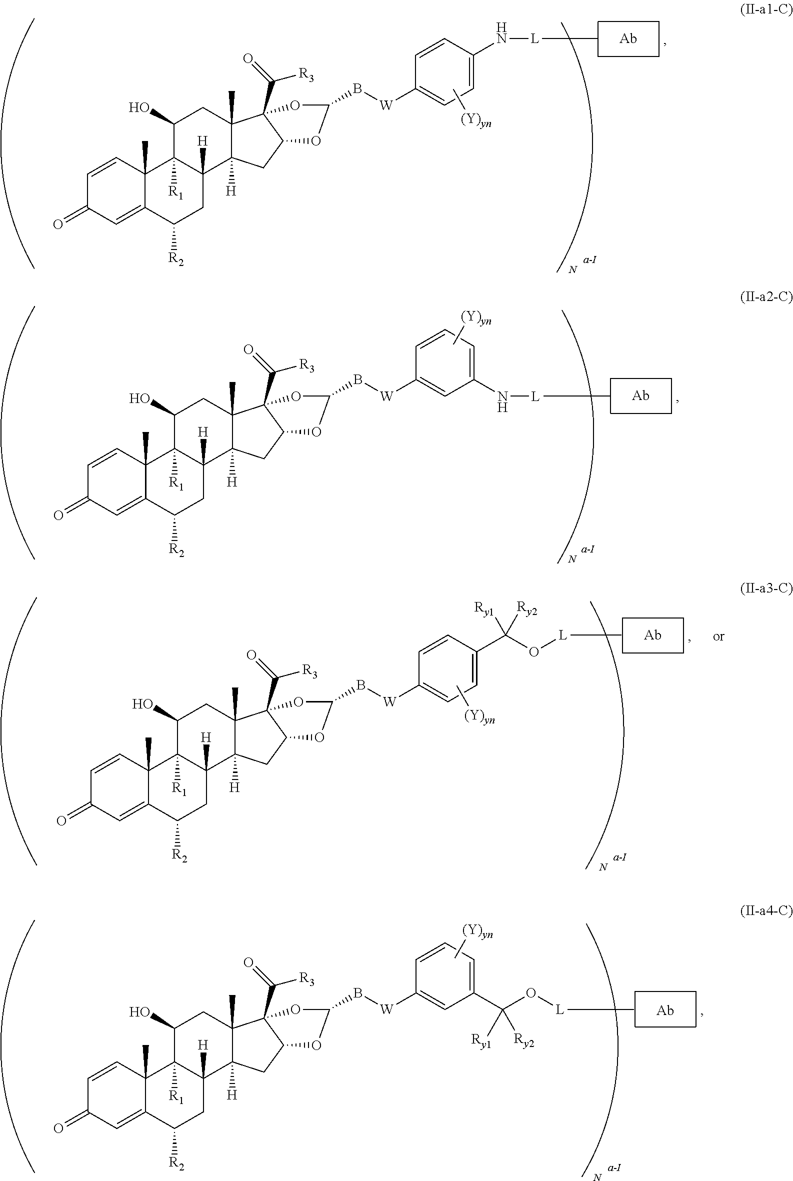
wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; L is absent or is any group; Ab is a ligand; N^{a-1} is a number equal to or greater than 0.

35. The compound or the tautomer, the mesomer, the racemate, the enantiomer or the diastereoisomer thereof, or the mixture thereof, or the pharmaceutically acceptable salt thereof according to any one of claims 31-34, wherein
L comprises Tr.

36. A compound or a tautomer, a mesomer, a racemate, an enantiomer or a diastereoisomer thereof, or a mixture thereof, or a pharmaceutically acceptable salt thereof, wherein the compound comprises a structure shown as formula (II-D): 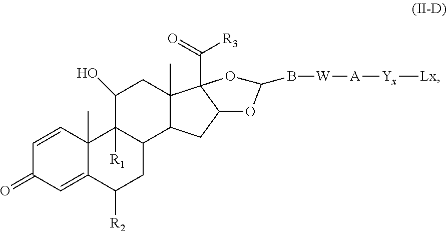
wherein
R₁, R₂ and R₃ are each independently any group;
B is selected from the group consisting of: optionally substituted alcylene, optionally substituted aliphatic heterocyclylene and optionally substituted C₂-C₄ heteroarylene;
W is absent or W is any group;
A is any group, wherein A is further substituted with one or more Y, and Y is any group; Yₓ is selected from the group consisting of: —O—, optionally substituted —CH₂—O—, —S—, optionally substituted —PH—, optionally substituted —P(═O)H—, and optionally substituted —NH—; Lx is any group.

37. A compound or a tautomer, a mesomer, a racemate, an enantiomer or a diastereoisomer thereof, or a mixture thereof, or a pharmaceutically acceptable salt thereof, wherein the compound comprises a structure shown as formula (II-a1-D), formula (II-a2-D), formula (II-a3-D) or formula (II-a4-D): 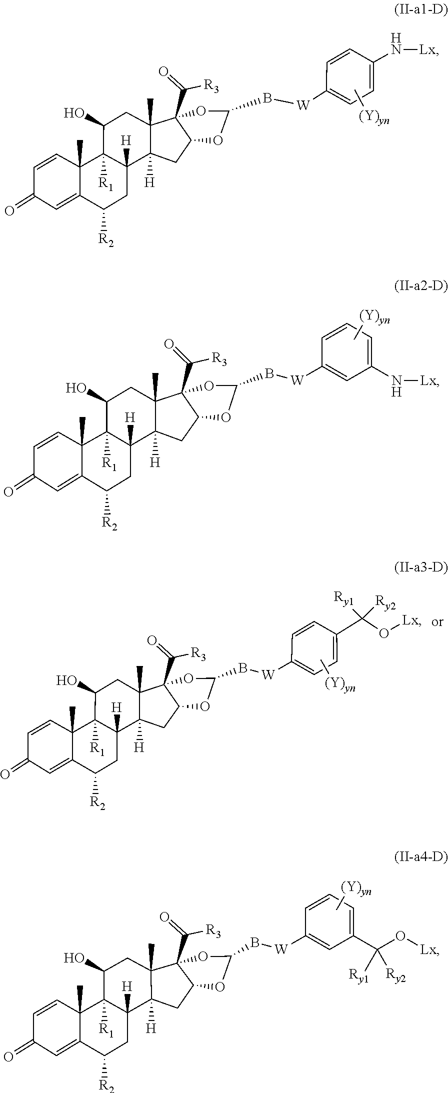
wherein
R₁, R₂, R₃, Ry₁, Ry₂, and Y are each independently any group;
B is selected from the group consisting of: optionally substituted alcylene, optionally substituted aliphatic heterocyclylene and optionally substituted C₂-C₄ heteroarylene; W is absent or W is any group; yn is an integer from 0 to 4; Lx is any group.

38. The compound or the tautomer, the mesomer, the racemate, the enantiomer or the diastereoisomer thereof, or the mixture thereof, or the pharmaceutically acceptable salt thereof according to any one of claims 36-37, wherein
Lx comprises Tr.

39. The compound or the tautomer, the mesomer, the racemate, the enantiomer or the diastereoisomer thereof, or the mixture thereof, or the pharmaceutically acceptable salt thereof according to any one of claims 29-30, 35 and 38, wherein
Tr comprises —(SP¹)ₙ₁—, wherein
each SP¹ is independently optionally substituted —CH₂—NH—, and
n1 is a number equal to or greater than 0.

40. The compound or the tautomer, the mesomer, the racemate, the enantiomer or the diastereoisomer thereof, or the mixture thereof, or the pharmaceutically acceptable salt thereof according to claim 39, wherein
each SP¹ is independently selected from the group consisting of: —CH₂—NH—, —CH(CH₃)—NH—, —C(CH₃)₂—NH—, —CH₂—N(CH₃)—, —CH(CH₃)—N(CH₃)— and —C(CH₃)₂—N(CH₃)—.

41. The compound or the tautomer, the mesomer, the racemate, the enantiomer or the diastereoisomer thereof, or the mixture thereof, or the pharmaceutically acceptable salt thereof according to any one of claims 29-38, wherein
R₁ can be selected from the group consisting of: hydrogen, halogen, and optionally substituted methyl;
R₂ can be selected from the group consisting of: hydrogen, halogen, and optionally substituted methyl;
R₃ can be selected from the group consisting of: optionally substituted —CH₂Cl, optionally substituted —CH₂SH, optionally substituted —CH₂OH, optionally substituted 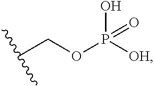 [SMILES: *COP(=O)(O)O]
optionally substituted 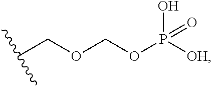 [SMILES: *COCOP(=O)(O)O]
optionally substituted 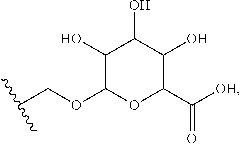 [SMILES: *COC1OC(C(=O)O)C(O)C(O)C1O]
optionally substituted 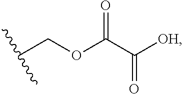 [SMILES: *COC(=O)C(=O)O]
optionally substituted 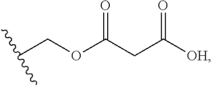 [SMILES: *COC(=O)CC(=O)O]
optionally substituted 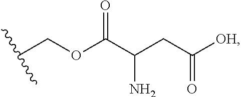 [SMILES: *COC(=O)C(N)CC(=O)O]
optionally substituted 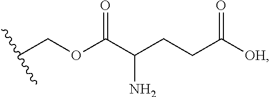 [SMILES: *COC(=O)C(N)CCC(=O)O]
optionally substituted 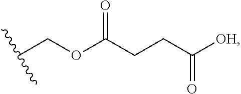 [SMILES: *COC(=O)CCC(=O)O]
optionally substituted 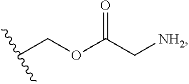 [SMILES: *COC(=O)CN]
optionally substituted 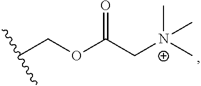 [SMILES: *COC(=O)C[N+](C)(C)C]
optionally substituted 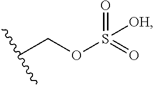 [SMILES: *COS(=O)(=O)O]
optionally substituted 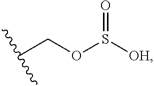 [SMILES: *COS(=O)O]
optionally substituted 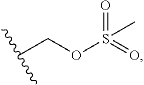 [SMILES: *COS(C)(=O)=O]
optionally substituted —OH, optionally substituted —OCH₃, optionally substituted —OCH₂F, optionally substituted —OCH₂Cl, optionally substituted —OCH₂CN, optionally substituted —OCH₂CH₃, optionally substituted sulfhydryl, optionally substituted —SCH₂F, optionally substituted —SCH₂Cl, optionally substituted —SCH₂CF₃ and optionally substituted —SCH₂CN;
B can be selected from the group consisting of: optionally substituted 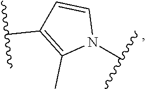 [SMILES: *c1ccn(*)c1C]
optionally substituted 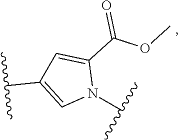 [SMILES: *c1cc(C(=O)OC)n(*)c1]
optionally substituted 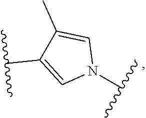 [SMILES: *c1cn(*)cc1C]
optionally substituted 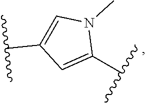 [SMILES: *c1cc(*)n(C)c1]
optionally substituted 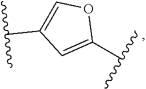 [SMILES: *c1coc(*)c1]
optionally substituted 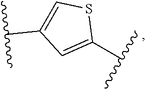 [SMILES: *c1csc(*)c1]
optionally substituted 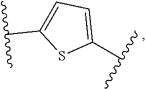 [SMILES: *c1ccc(*)s1]
optionally substituted 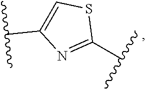 [SMILES: *c1csc(*)n1]
and optionally substituted 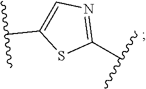 [SMILES: *c1cnc(*)s1]
W can be absent or W can be selected from the group consisting of the following structures that are optionally substituted: 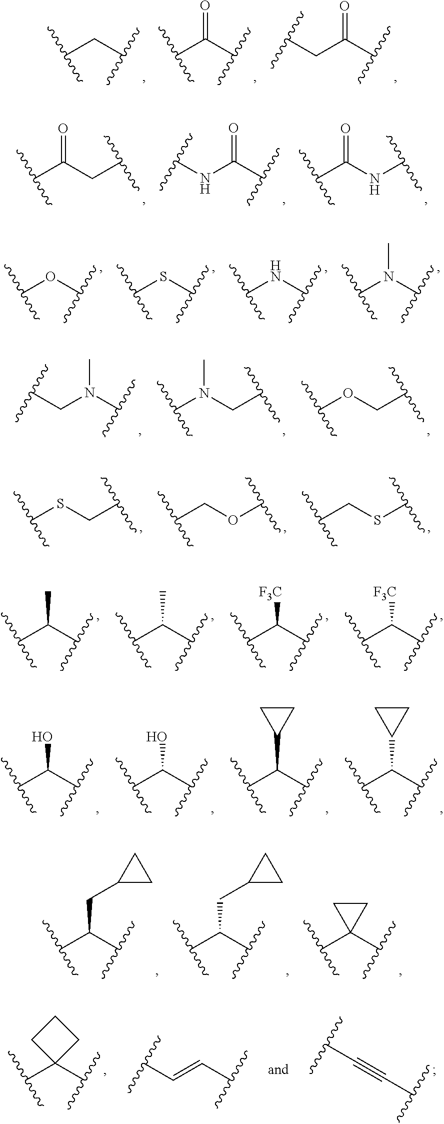
Y can be H, and yn can be a number greater than 0;
Tr can be absent or selected from the group consisting of: 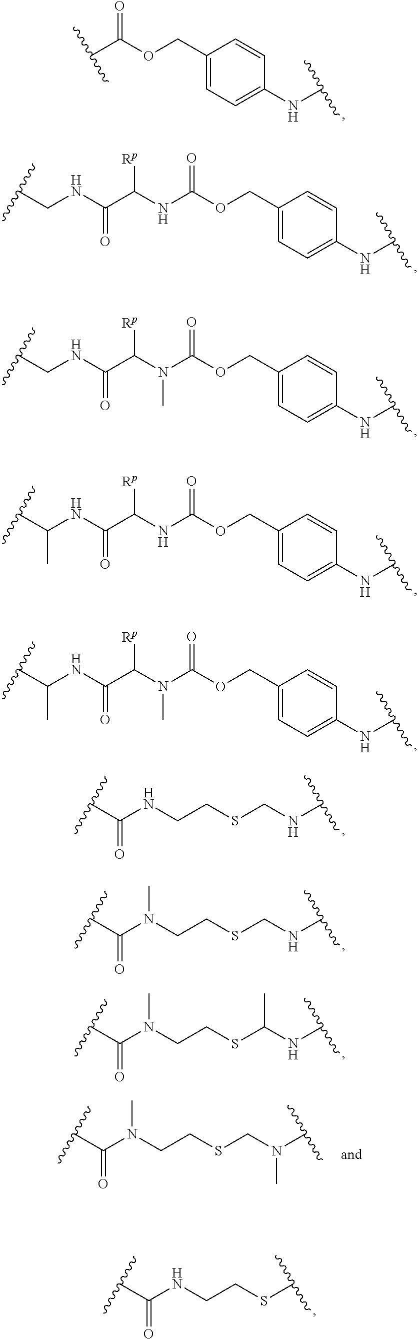
wherein R^{P} can be selected from the group consisting of: H, —CH₃, —CH—(CH₃)₂, —CH₂—CH(CH₃)₂, —CH(CH₃)—CH₂—CH₃, —CH₂—C₆H₅, —CsNH₆, —CH₂—C₆H₄—OH, —CH₂—COOH, —CH₂—CONH₂, —(CH₂)₂—COOH, —(CH₂)₄—NH₂, —(CH₂)₂—CONH₂, —(CH₂)₂—S—CH₃, —CH₂—OH, —CH(CH₃)—OH, —CH₂—SH, —C₃H₆, —CH₂—C₃H₃N, —(CH₂)₃—NHC(NH—)NH₂ and —(CH₂)₃—NHCONH2.

42. The compound or the tautomer, the mesomer, the racemate, the enantiomer or the diastereoisomer thereof, or the mixture thereof, or the pharmaceutically acceptable salt thereof according to any one of claims 29-38,
wherein,
R₁ can be selected from the group consisting of: hydrogen and halogen;
R₂ can be selected from the group consisting of: hydrogen and halogen;
R₃ can be selected from the group consisting of: —CH₂OH, —SCH₂-halogen, 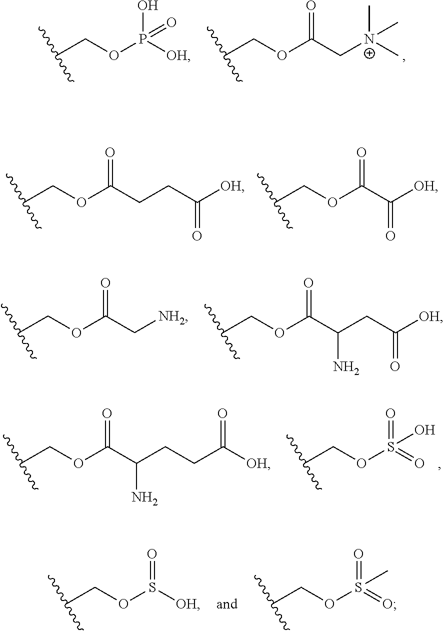
B can be selected from the group consisting of: optionally substituted 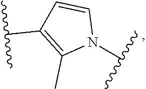 [SMILES: *c1ccn(*)c1C]
optionally substituted 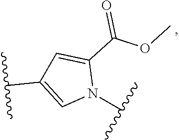 [SMILES: *c1cc(C(=O)OC)n(*)c1]
optionally substituted 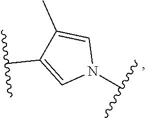 [SMILES: *c1cn(*)cc1C]
optionally substituted 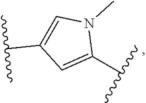 [SMILES: *c1cc(*)n(C)c1]
optionally substituted 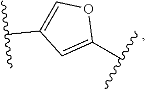 [SMILES: *c1coc(*)c1]
optionally substituted 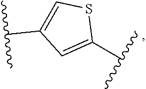 [SMILES: *c1csc(*)c1]
optionally substituted 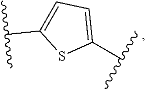 [SMILES: *c1ccc(*)s1]
optionally substituted 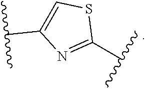 [SMILES: *c1csc(*)n1]
and optionally substituted 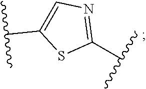 [SMILES: *c1cnc(*)s1]
W can be absent or W can be selected from the group consisting of the following structures that are optionally substituted: optionally substituted —CH₂—, optionally substituted —CH(CH₃)—, and optionally substituted 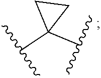 [SMILES: *C1(*)CC1]
Y is H, and yn can be a number greater than 0;
Tr is absent.

43. The compound or the tautomer, the mesomer, the racemate, the enantiomer or the diastereoisomer thereof, or the mixture thereof, or the pharmaceutically acceptable salt thereof according to any one of claims 36-38, wherein
Lx comprises L^{1x}, and L^{1x} is selected from the group consisting of: a group capable of coupling with amino, a group capable of coupling with sulfhydryl, and a click chemistry group.

44. The compound or the tautomer, the mesomer, the racemate, the enantiomer or the diastereoisomer thereof, or the mixture thereof, or the pharmaceutically acceptable salt thereof according to claim 43, wherein
L^{1x} is selected from the group consisting of: 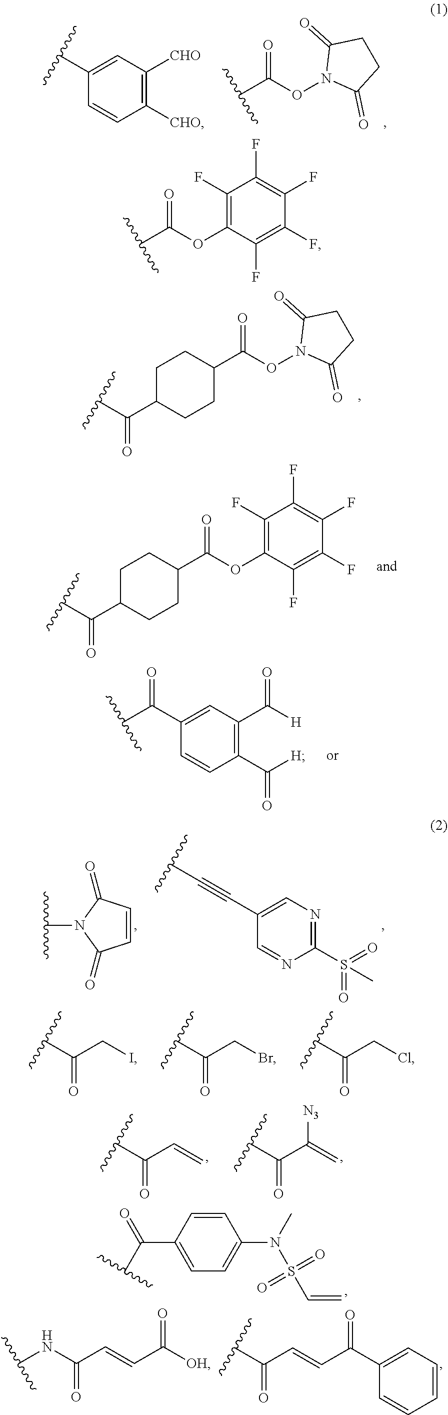 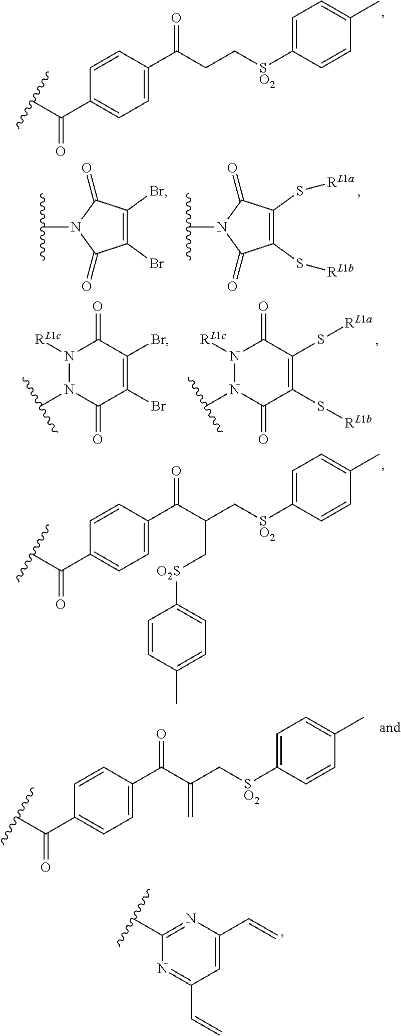
wherein each R^{L1a}, each R^{L1b} and each R^{L1c} are each independently selected from any group; or

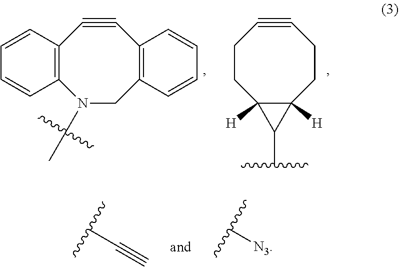

45. The compound or the tautomer, the mesomer, the racemate, the enantiomer or the diastereoisomer thereof, or the mixture thereof, or the pharmaceutically acceptable salt thereof according to any one of claims 31-35, wherein
L comprises L¹, and L¹ is selected from the group consisting of: a divalent or trivalent residue formed by participation of amino in coupling, a divalent or trivalent residue formed by participation of sulfhydryl in coupling, and a divalent or trivalent residue formed by click chemistry coupling.

46. The compound or the tautomer, the mesomer, the racemate, the enantiomer or the diastereoisomer thereof, or the mixture thereof, or the pharmaceutically acceptable salt thereof according to claim 45, wherein
L¹ is selected from the group consisting of: 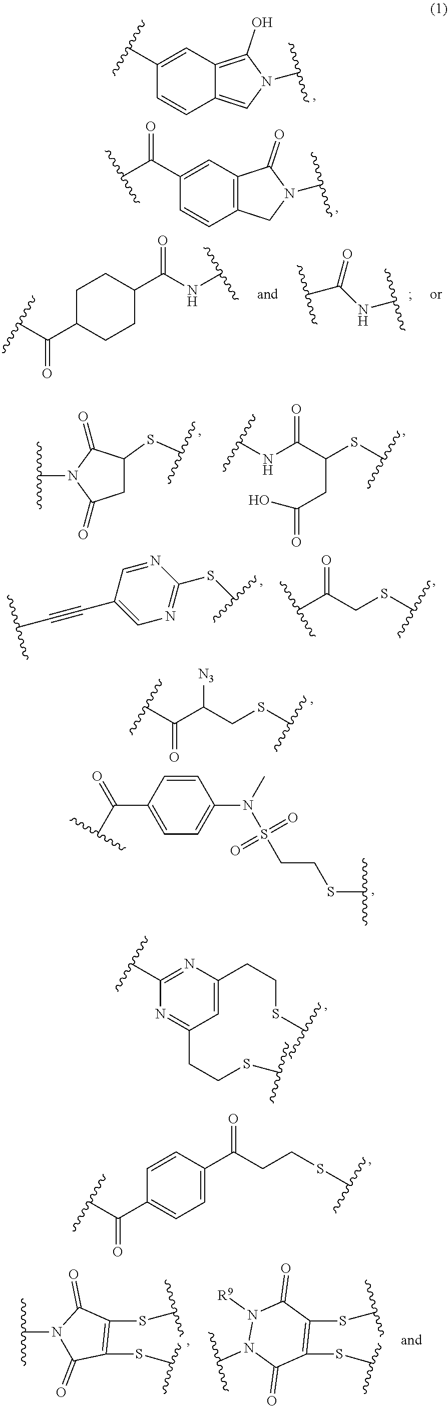 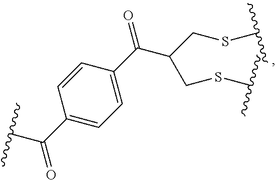
wherein R⁹ is selected from hydrogen and C₁-C₆ alkyl; or

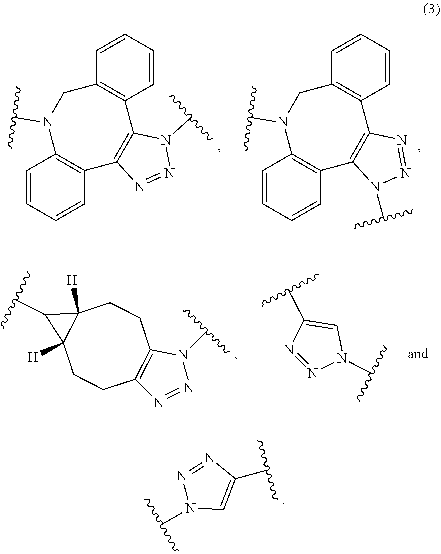 [SMILES: *N1Cc2ccccc2-c2c(nnn2*)-c2ccccc21 ; *N1Cc2ccccc2-c2nnn(*)c2-c2ccccc21 ; *C1[C@H]2CCc3nnn(*)c3CC[C@@H]12 ; *c1cn(*)nn1 ; *c1cn(*)nn1]

47. The compound or the tautomer, the mesomer, the racemate, the enantiomer or the diastereoisomer thereof, or the mixture thereof, or the pharmaceutically acceptable salt thereof according to any one of claims 31-46, wherein
L or Lx comprises L², and L² is any group.

48. The compound or the tautomer, the mesomer, the racemate, the enantiomer or the diastereoisomer thereof, or the mixture thereof, or the pharmaceutically acceptable salt thereof according to claim 47, wherein
L² comprises —X—, and X is optionally substituted —(CH₂)ₚ₁—,
wherein each methylene unit of X is unreplaced, or at least 1 methylene unit of X is each independently replaced by a group selected from any group, and p1 is a number equal to or greater than 0.

49. The compound or the tautomer, the mesomer, the racemate, the enantiomer or the diastereoisomer thereof, or the mixture thereof, or the pharmaceutically acceptable salt thereof according to claim 48, wherein
X is selected from the group consisting of: —C(O)—, —OC(O)—, —C(O)O—, —NR^{x}— and —S—C(R^{xa})(R^{xb})—CH₂—NH—C(O)—, wherein each R^{x}, each R^{xa} and each R^{xb} are each independently selected from the group consisting of: hydrogen and alkyl.

50. The compound or the tautomer, the mesomer, the racemate, the enantiomer or the diastereoisomer thereof, or the mixture thereof, or the pharmaceutically acceptable salt thereof according to any one of claims 47-49, wherein
L² further comprises -LB-, and
LB is 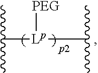
wherein L^{p} is a trivalent residue, PEG comprises a polyethylene glycol unit, and p2 is a number equal to or greater than 0.

51. The compound or the tautomer, the mesomer, the racemate, the enantiomer or the diastereoisomer thereof, or the mixture thereof, or the pharmaceutically acceptable salt thereof according to claim 50, wherein
p2 is selected from the group consisting of: 0, 1, 2, 3, 4 and 5.

52. The compound or the tautomer, the mesomer, the racemate, the enantiomer or the diastereoisomer thereof, or the mixture thereof, or the pharmaceutically acceptable salt thereof according to any one of claims 50-51, wherein
L^{p} is selected from the group consisting of: aspartic acid, glutamic acid, histidine, lysine, arginine, serine, cysteine, threonine, and tyrosine.

53. The compound or the tautomer, the mesomer, the racemate, the enantiomer or the diastereoisomer thereof, or the mixture thereof, or the pharmaceutically acceptable salt thereof according to any one of claims 50-52, wherein
PEG is selected from the group consisting of:

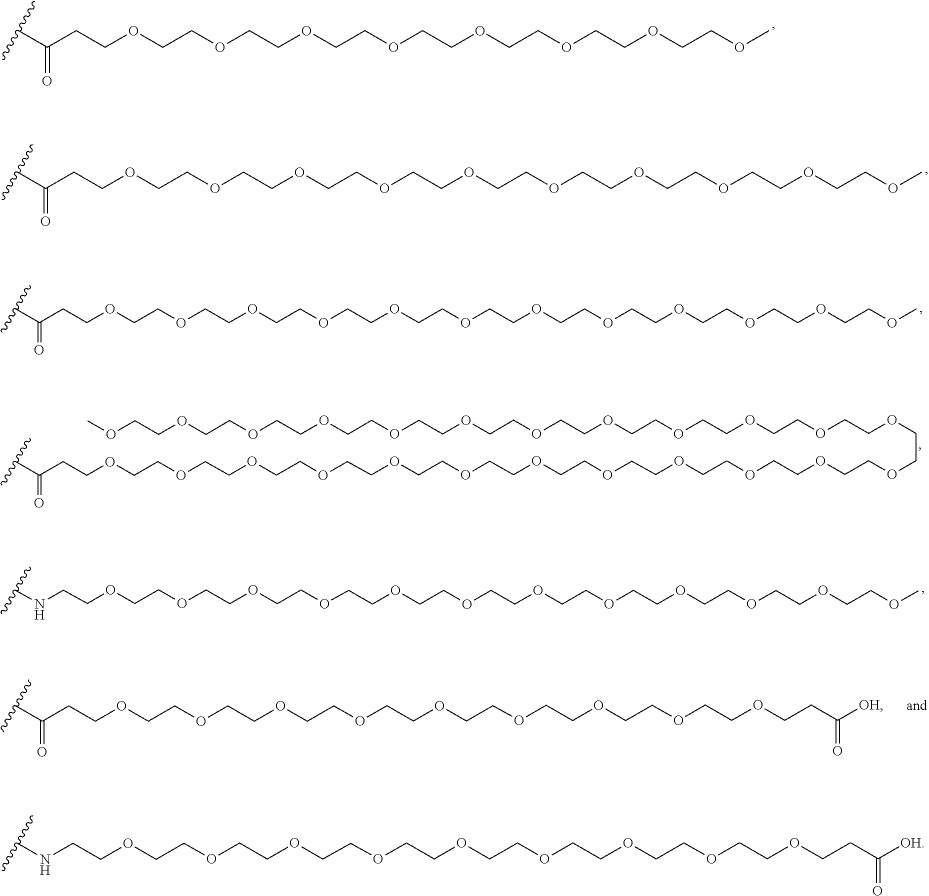

54. The compound or the tautomer, the mesomer, the racemate, the enantiomer or the diastereoisomer thereof, or the mixture thereof, or the pharmaceutically acceptable salt thereof according to any one of claims 50-53, wherein
LB is selected from the group consisting of:

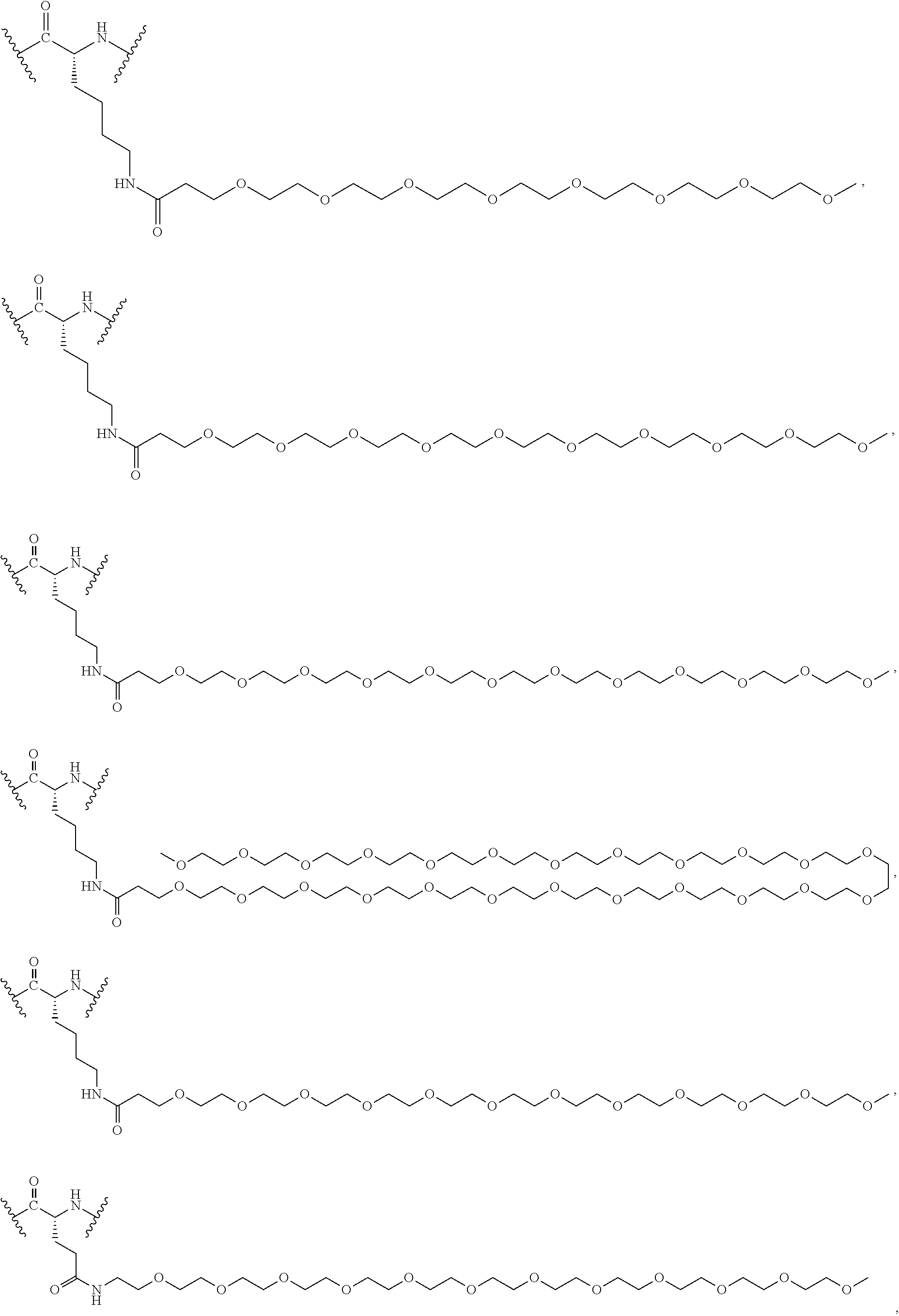 [SMILES: *N[C@H](CCCCNC(=O)CCOCCOCCOCCOCCOCCOCCOCCOC)C(*)=O ; *N[C@H](CCCCNC(=O)CCOCCOCCOCCOCCOCCOCCOCCOCCOCCOC)C(*)=O ; *N[C@H](CCCCNC(=O)CCOCCOCCOCCOCCOCCOCCOCCOCCOCCOCCOCCOC)C(*)=O ; *N[C@H](CCCCNC(=O)CCOCCOCCOCCOCCOCCOCCOCCOCCOCCOCCOCCOCCOCCOCCOCCOCCOCCOCCOCCOCCOCCOCCOCCOC)C(*)=O ; *N[C@H](CCCCNC(=O)CCOCCOCCOCCOCCOCCOCCOCCOCCOCCOCCOCCOC)C(*)=O ; *N[C@H](CCC(=O)NCCOCCOCCOCCOCCOCCOCCOCCOCCOCCOCCOCCOC)C(*)=O]

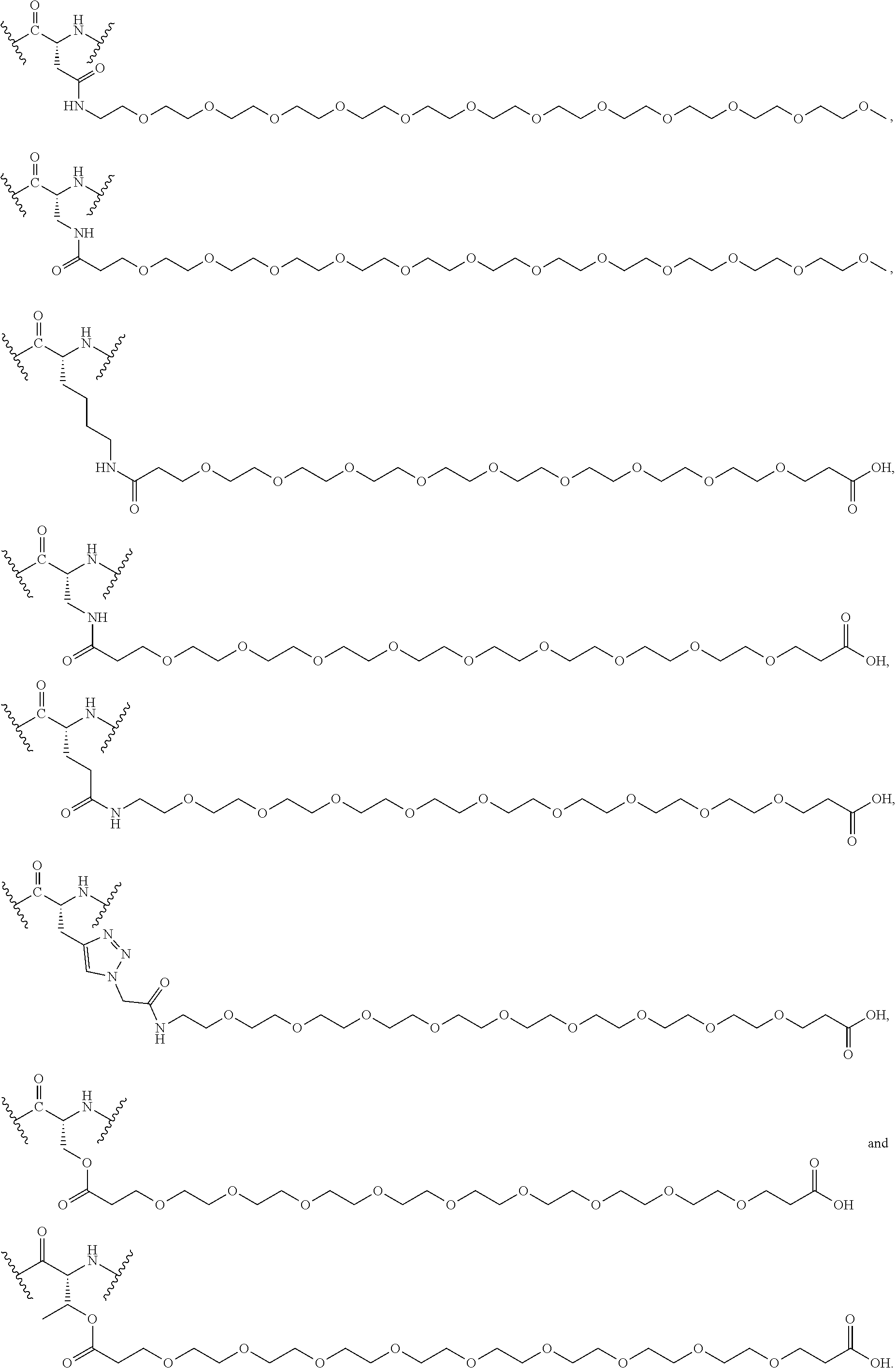

55. The compound or the tautomer, the mesomer, the racemate, the enantiomer or the diastereoisomer thereof, or the mixture thereof, or the pharmaceutically acceptable salt thereof according to any one of claims 47-54, wherein
L² further comprises -LY-, and
LY is —Oₚ₋₇—(CH₂CH₂O)ₚ₋₆—, wherein p-6 and p-7 are each independently at least 0.

56. The compound or the tautomer, the mesomer, the racemate, the enantiomer or the diastereoisomer thereof, or the mixture thereof, or the pharmaceutically acceptable salt thereof according to any one of claim 55, wherein
LY is selected from the group consisting of: —(CH₂CH₂O)₃—, —(CH₂CH₂O)₄—, —(CH₂CH₂O)₅—, —(CH₂CH₂O)₆—, —(CH₂CH₂O)₇— and —(CH₂CH₂O)₈—.

57. The compound or the tautomer, the mesomer, the racemate, the enantiomer or the diastereoisomer thereof, or the mixture thereof, or the pharmaceutically acceptable salt thereof according to any one of claims 47-56, wherein
L² further comprises —Z—, and
Z is optionally substituted —(CH₂)ₚ₋₈—,
wherein each methylene unit of Z is unreplaced, or at least 1 methylene unit of Z is each independently replaced by a group selected from any group, and p-8 is at least 0.

58. The compound or the tautomer, the mesomer, the racemate, the enantiomer or the diastereoisomer thereof, or the mixture thereof, or the pharmaceutically acceptable salt thereof according to claim 57, wherein
Z is selected from the group consisting of: optionally substituted —NH—, optionally substituted —(CH₂)₂—, optionally substituted —(CH₂)₅—, optionally substituted —C(O)—(CH₂)₂—, optionally substituted —C(O)—(CH₂)₄—, optionally substituted —C(O)—(CH₂)₅—, optionally substituted —(CH₂)₂—NH—C(O)—(CH₂)₂—, optionally substituted —(CH₂)₂—NH—C(O)—(CH₂)₂—C(O)—, optionally substituted —(CH₂)₂—NH— and optionally substituted —(CH₂)₂—NH—C(O)—O—CH₂—.

59. The compound or the tautomer, the mesomer, the racemate, the enantiomer or the diastereoisomer thereof, or the mixture thereof, or the pharmaceutically acceptable salt thereof according to any one of claims 47-58, wherein
L² is absent.

60. The compound or the tautomer, the mesomer, the racemate, the enantiomer or the diastereoisomer thereof, or the mixture thereof, or the pharmaceutically acceptable salt thereof according to any one of claims 47-58, wherein
L² is selected from the group consisting of:

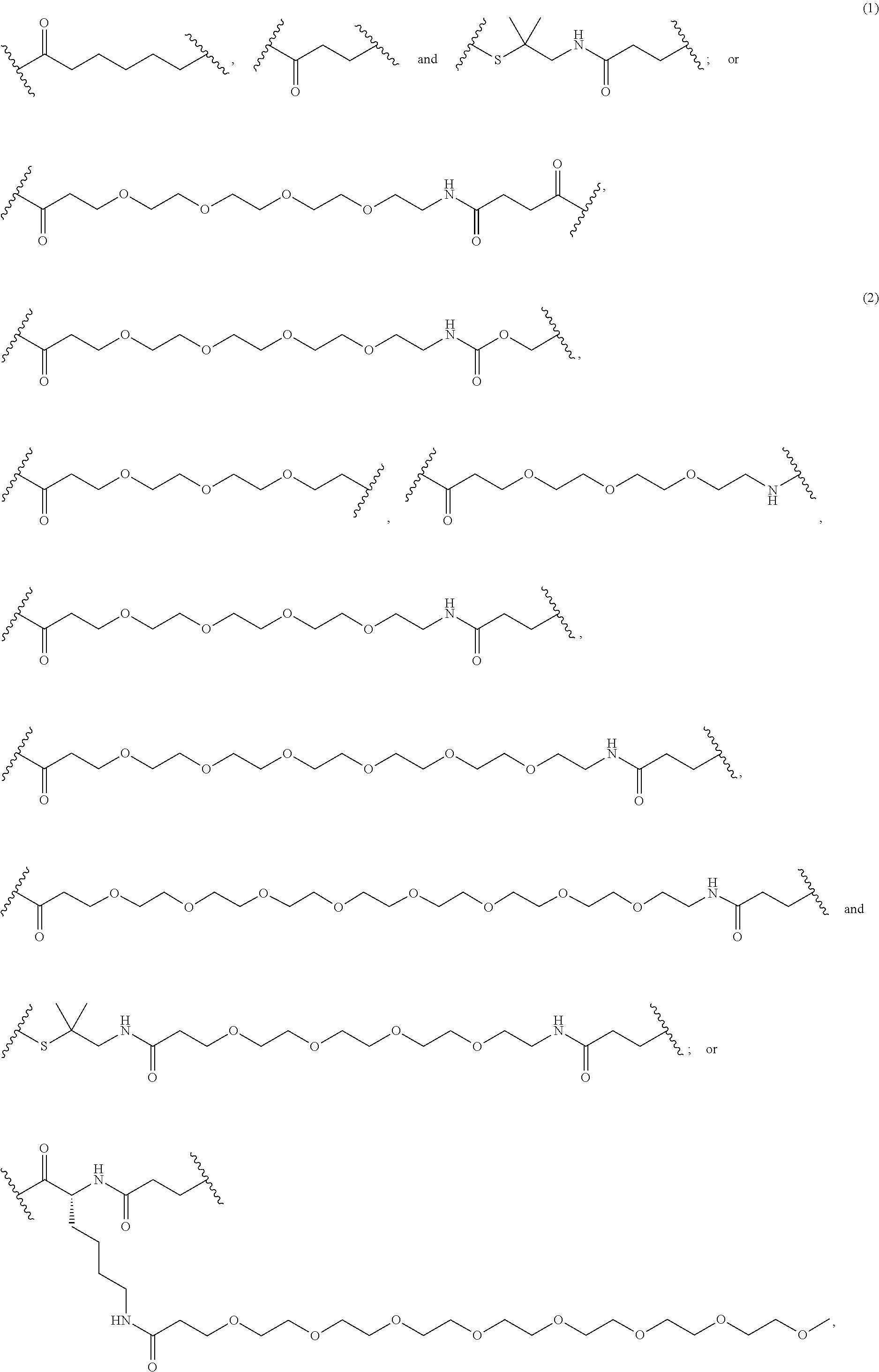

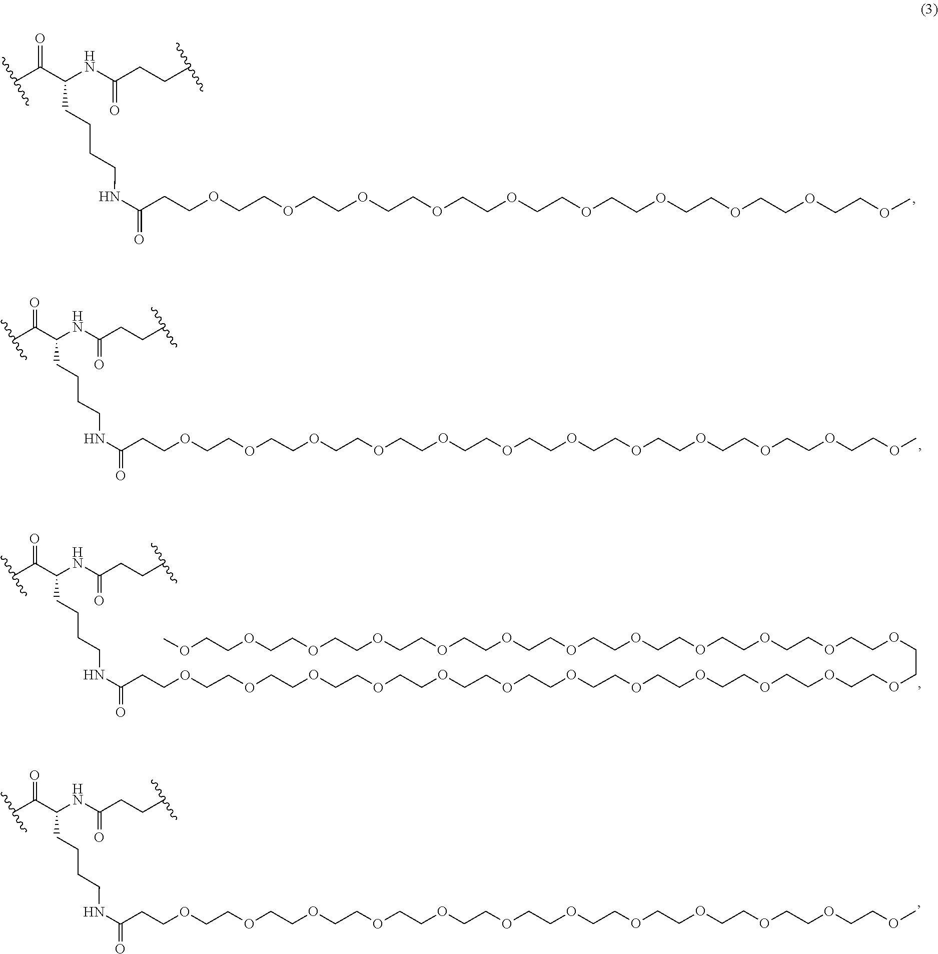 [SMILES: *CCC(=O)N[C@H](CCCCNC(=O)CCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOCCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]

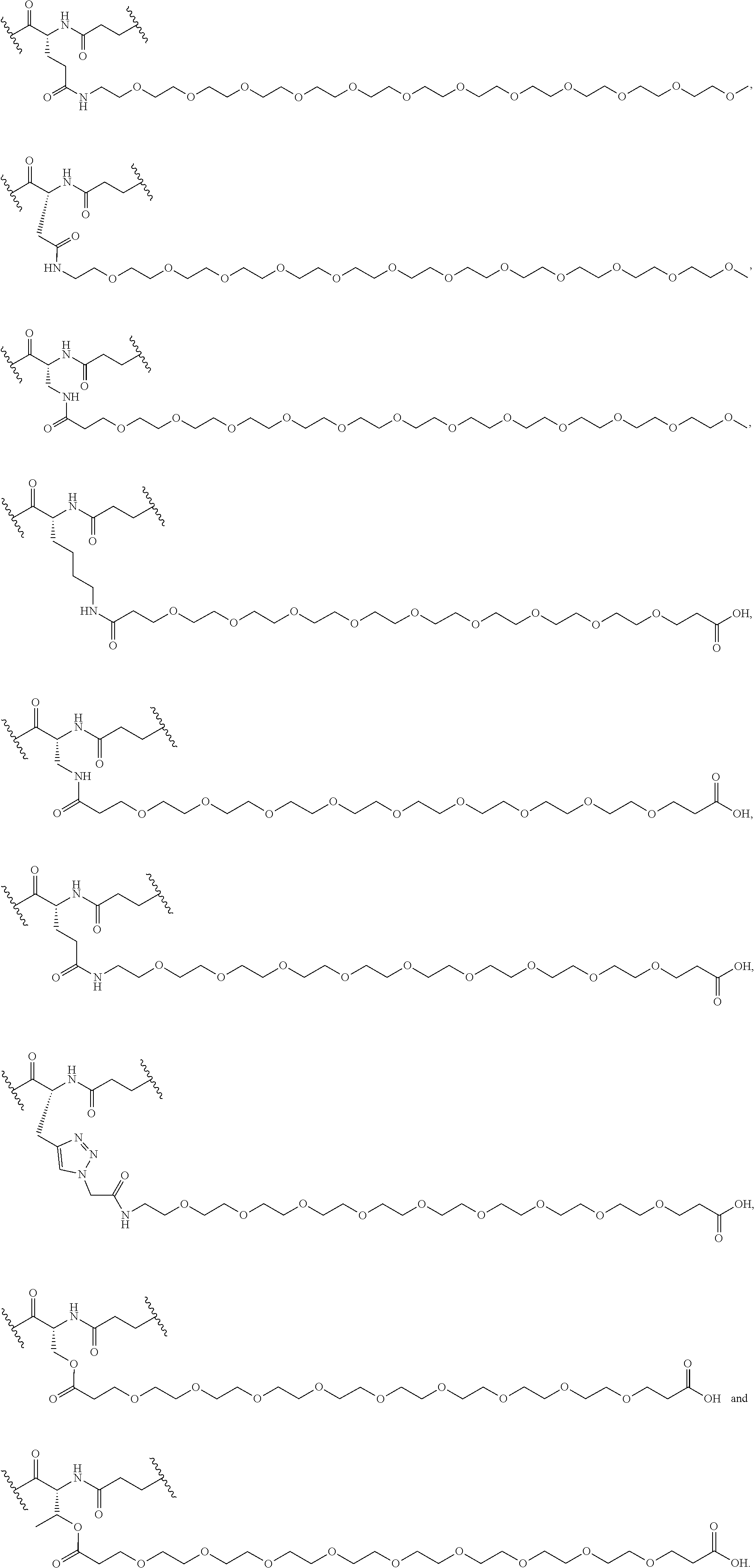

61. The compound or the tautomer, the mesomer, the racemate, the enantiomer or the diastereoisomer thereof, or the mixture thereof, or the pharmaceutically acceptable salt thereof according to any one of claims 31-60, wherein
L or Lx comprises L³, and L³ is a polypeptide residue.

62. The compound or the tautomer, the mesomer, the racemate, the enantiomer or the diastereoisomer thereof, or the mixture thereof, or the pharmaceutically acceptable salt thereof according to claim 61, wherein
L³ is selected from the group consisting of: glutamic acid-glycine (EG), glycine-glutamic acid (GE), glutamic acid-alanine (EA), alanine-glutamic acid (AE), valine-citrulline (VC), citrulline-valine (CV), valine-alanine (VA), alanine-valine (AV), alanine-alanine (AA), glutamic acid-alanine-glycine (EAG), glycine-alanine-glutamic acid (GAE), glutamic acid-glycine-glycine (EGG), glycine-glycine-glutamic acid (GGE), glycine-glutamic acid-glycine (GEG), alanine-alanine-glycine (AAG), glycine-alanine-alanine (GAA), alanine-alanine-alanine (AAA), valine-alanine-glycine (VAG), glycine-alanine-valine (GAV), valine-citrulline-glycine (VCG), glycine-citrulline-valine (GCV), valine-lysine-glycine (VKG), glycine-lysine-valine (GKV), glycine-glycine-phenylalanine-glycine (GGFG), glycine-phenylalanine-glycine-glycine (GFGG), glycine-glycine-glycine-glycine (GGGG), glycine-glutamic acid-glycine-glycine (GEGG), glycine-glycine-glutamic acid-glycine (GGEG), glycine-alanine-glutamic acid-glycine (GAEG) and glycine-glutamic acid-alanine-glycine (GEAG).

63. The compound or the tautomer, the mesomer, the racemate, the enantiomer or the diastereoisomer thereof, or the mixture thereof, or the pharmaceutically acceptable salt thereof according to any one of claims 61-62, wherein
L³ is selected from the group consisting of: alanine-alanine (AA), glycine-alanine-glutamic acid (GAE), glycine-glutamic acid-glycine (GEG), alanine-glutamic acid (AE), glutamic acid-glycine (EG), and glycine-glutamic acid (GE).

64. The compound or the tautomer, the mesomer, the racemate, the enantiomer or the diastereoisomer thereof, or the mixture thereof, or the pharmaceutically acceptable salt thereof according to any one of claims 31-63, wherein
L comprises -L³-L²-L¹- or -L³-L¹-;
preferably, L is selected from

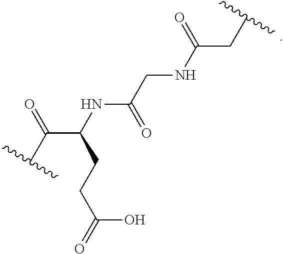 [SMILES: *CC(=O)NCC(=O)N[C@@H](CCC(=O)O)C(*)=O]

65. The compound or the tautomer, the mesomer, the racemate, the enantiomer or the diastereoisomer thereof, or the mixture thereof, or the pharmaceutically acceptable salt thereof according to any one of claims 36-63, wherein
Lx comprises -L³-L²-L^{1x} or -L³-L^{1x};
preferably, Lx is selected from

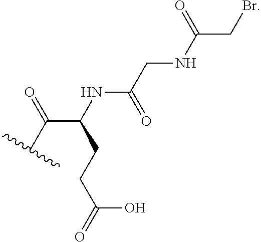 [SMILES: *C(=O)[C@H](CCC(=O)O)NC(=O)CNC(=O)CBr]

66. The compound or the tautomer, the mesomer, the racemate, the enantiomer or the diastereoisomer thereof, or the mixture thereof, or the pharmaceutically acceptable salt thereof according to any one of claims 33-35, wherein
Ab comprises an antibody or an antigen-binding fragment thereof.

67. The compound or the tautomer, the mesomer, the racemate, the enantiomer or the diastereoisomer thereof, or the mixture thereof, or the pharmaceutically acceptable salt thereof according to claim 66, wherein
the antibody is selected from the group consisting of: a murine antibody, a chimeric antibody, a humanized antibody and a fully humanized antibody.

68. The compound or the tautomer, the mesomer, the racemate, the enantiomer or the diastereoisomer thereof, or the mixture thereof, or the pharmaceutically acceptable salt thereof according to claim 66, wherein
the antigen-binding fragment is selected from the group consisting of: a Fab, a Fab′, a Fv fragment, a F(ab′)₂, a F(ab)₂, a scFv, a di-scFv, a VHH and a dAb.

69. The compound or the tautomer, the mesomer, the racemate, the enantiomer or the diastereoisomer thereof, or the mixture thereof, or the pharmaceutically acceptable salt thereof according to any one of claims 66-68, wherein
N^{a-1} is a number from about 0 to about 20.

70. The compound or the tautomer, the mesomer, the racemate, the enantiomer or the diastereoisomer thereof, or the mixture thereof, or the pharmaceutically acceptable salt thereof according to any one of claims 66-69, wherein
Ab targets a target selected from the group consisting of:
AXL, BAFFR, BCMA, BCR-list components, BDCA2, BDCA4, BTLA, BTNL2 BTNL3, BTNL8, BTNL9, C10orf54, CCR1, CCR3, CCR4, CCR5, CCR6, CCR7, CCR9, CCR10, CD11c, CD137, CD138, CD14, CD163, CD168, CD177, CD19, CD20, CD209, CD209L, CD22, CD226, CD248, CD25, CD27, CD274, CD276, CD28, CD30, CD300A, CD33, CD37, CD38, CD4, CD40, CD44, CD45, CD46, CD47, CD48, CD5, CD52, CD55, CD56, CD59, CD62E, CD68, CD69, CD70, CD74, CD79a, CD79b, CD8, CD80, CD86, CD90.2, CD96, CLEC12A, CLEC12B, CLEC7A, CLEC9A, CR1, CR3, CRTAM, CSF1R, CTLA4, CXCR1/2, CXCR4, CXCR5, DDR1, DDR2, DEC-205, DLL4, DR6, FAP, FCamR, FCMR, FcR's, Fire, GITR, HHLA2, HLA class II, HVEM, ICOSLG, IFNAR, IFNLR1, IL10R1, IL10R2, IL12R, IL13RA1, IL13RA2, IL15R, IL17RA, IL17RB, IL17RC, IL17RE, IL20R1, IL20R2, IL21R, IL22R1, IL22RA, IL23R, IL27R, IL29R, IL2Rg, IL31R, IL36R, IL3RA, IL4R, IL6R, IL5R, IL7R, IL9R, integrins, LAG3, LIFR, MAG/Siglec-4 (sialic acid-binding immunoglobulin-like lectin-4), MMR, MSR1, NCR3LG1, NKG2D, NKp30, NKp46, OX40 (CD134), PDCD1, PROKR1, PVR, PVRIG, PVRL2, PVRL3, RELT, SIGIRR, Siglec-1 (sialic acid-binding immunoglobulin-like lectin-1), Siglec-10, Siglec-5, Siglec-6, Siglec-7, Siglec-8, Siglec-9, SIRPA, SLAMF7, TACI, TCR-list components/assoc, PTCRA, TCRb, CD3z, CD3, TEK, TGFBR1, TGFBR2, TGFBR3, TIGIT, TLR2, TLR4, TNFα, TROY, TSLPR, TYRO, VLDLR, VSIG4, IL2R-y and VTCN1.

71. The compound or the tautomer, the mesomer, the racemate, the enantiomer or the diastereoisomer thereof, or the mixture thereof, or the pharmaceutically acceptable salt thereof according to any one of claims 66-70, wherein
Ab is selected from the group consisting of: an anti-TNFα antibody or an antigen-binding fragment thereof, an anti-CD40 antibody or an antigen-binding fragment thereof, an anti-BDCA2 antibody or an antigen-binding fragment thereof, and an anti-IFNAR1 antibody or an antigen-binding fragment thereof.

72. The compound or the tautomer, the mesomer, the racemate, the enantiomer or the diastereoisomer thereof, or the mixture thereof, or the pharmaceutically acceptable salt thereof according to any one of claims 66-71, wherein
the anti-TNFα antibody or the antigen-binding fragment thereof comprises a heavy chain variable region (VH) and a light chain variable region (VL) of an antibody, wherein the heavy chain variable region comprises a heavy chain variable region of adalimumab, infliximab, afelimomab or golimumab, and the light chain variable region comprises a light chain variable region of adalimumab, infliximab, afelimomab or golimumab.

73. The compound or the tautomer, the mesomer, the racemate, the enantiomer or the diastereoisomer thereof, or the mixture thereof, or the pharmaceutically acceptable salt thereof according to claim 72, wherein
the anti-TNFα antibody or the antigen-binding fragment thereof comprises a heavy chain and a light chain of an antibody, wherein the heavy chain comprises a heavy chain of adalimumab, infliximab, afelimomab or golimumab, and the light chain comprises a light chain of adalimumab, infliximab, afelimomab or golimumab.

74. The compound or the tautomer, the mesomer, the racemate, the enantiomer or the diastereoisomer thereof, or the mixture thereof, or the pharmaceutically acceptable salt thereof according to any one of claims 66-71, wherein
the anti-CD40 antibody or the antigen-binding fragment thereof comprises a heavy chain and a light chain of an antibody, wherein the heavy chain comprises a heavy chain of iscalimab (CFZ533), and the light chain comprises a light chain of iscalimab (CFZ533).

75. The compound or the tautomer, the mesomer, the racemate, the enantiomer or the diastereoisomer thereof, or the mixture thereof, or the pharmaceutically acceptable salt thereof according to any one of claims 66-71, wherein
the anti-IFNAR1 antibody or the antigen-binding fragment thereof comprises a heavy chain and a light chain of an antibody, wherein the heavy chain comprises a heavy chain of anifrolumab (MEDI-546), and the light chain comprises a light chain of anifrolumab (MEDI-546).

76. The compound or the tautomer, the mesomer, the racemate, the enantiomer or the diastereoisomer thereof, or the mixture thereof, or the pharmaceutically acceptable salt thereof according to any one of claims 66-71, wherein
the anti-BDCA2 antibody or the antigen-binding fragment thereof comprises a heavy chain and a light chain of an antibody, wherein the heavy chain comprises a heavy chain of litifilimab (BIIB059), and the light chain comprises a light chain of litifilimab (BIIB059).

77. A compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

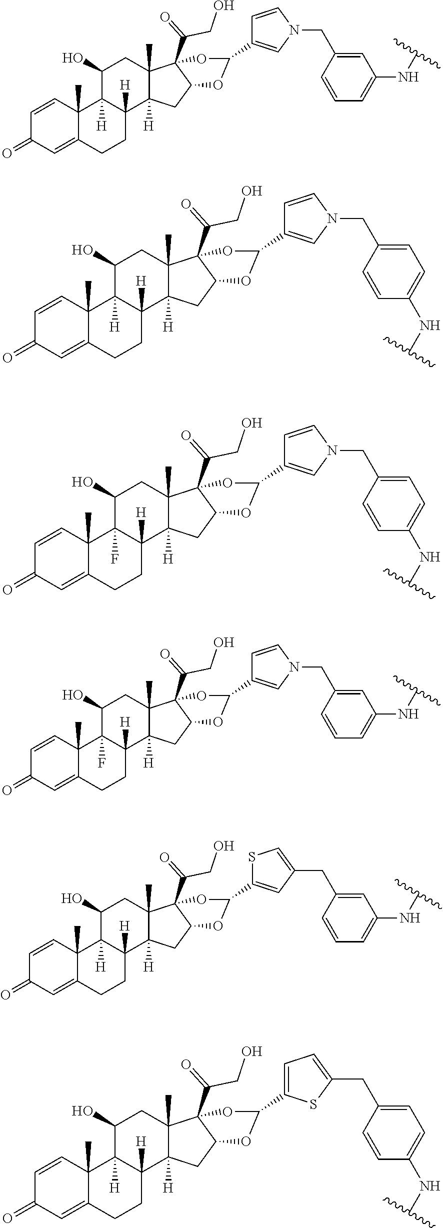

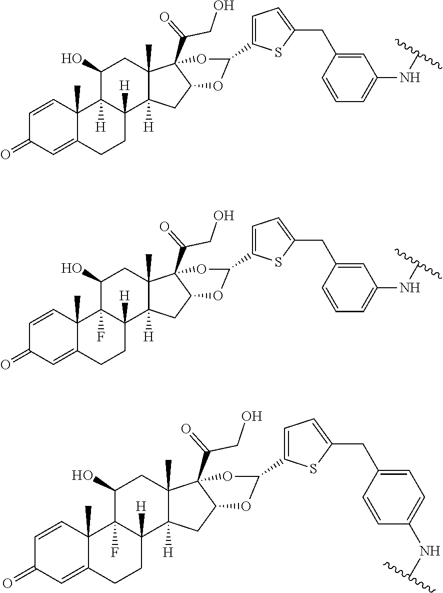

78. A compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

| No. | Structure |
|---|---|
| II- D-1 | 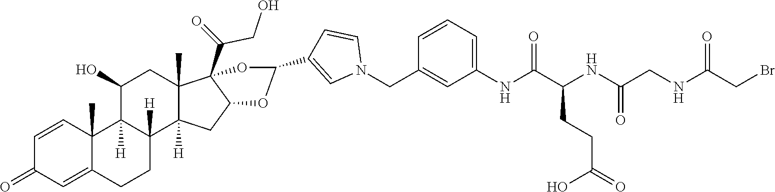 |
| II- D-2 | 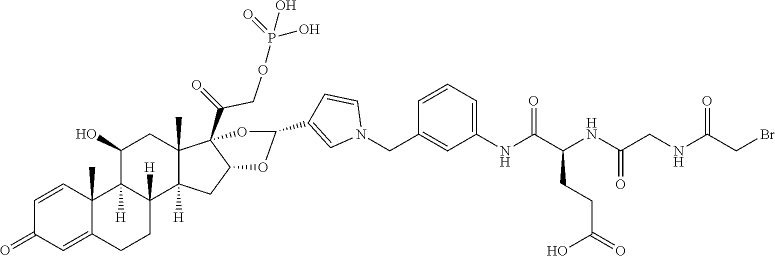 |
| II- D-7 | 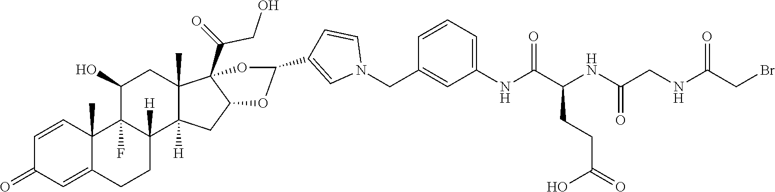 |
| II- D-8 | 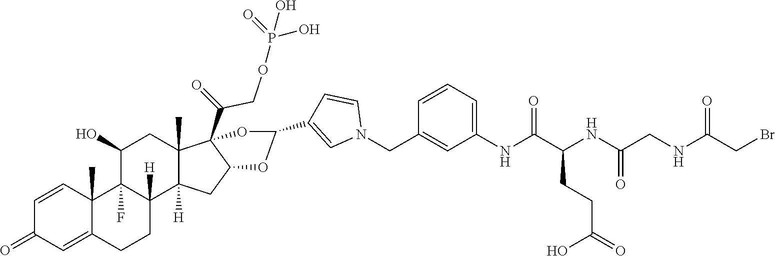 |
| II- D- 12 | 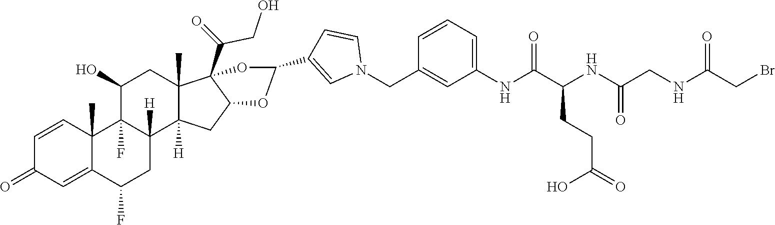 |
| II- D- 13 | 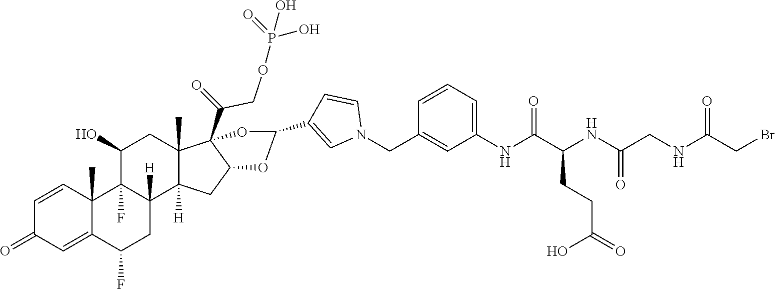 |
| II- D- 17 | 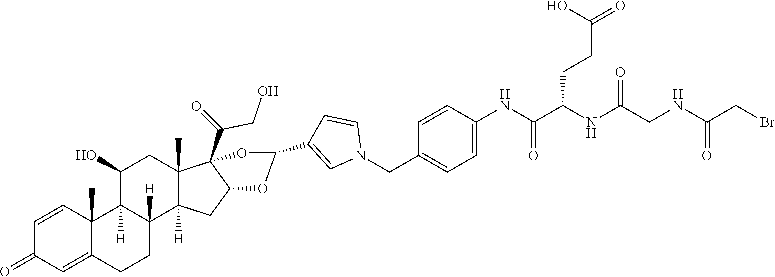 |
| II- D- 18 | 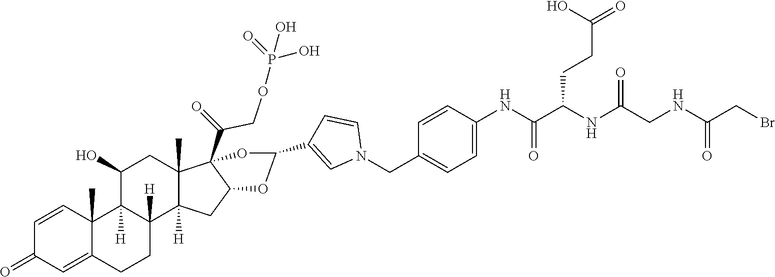 |
| II- D- 23 | 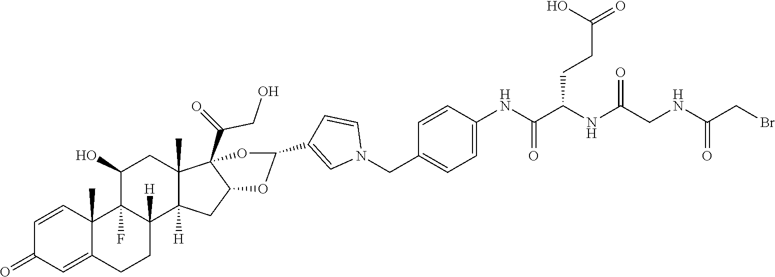 |
| II- D- 24 | 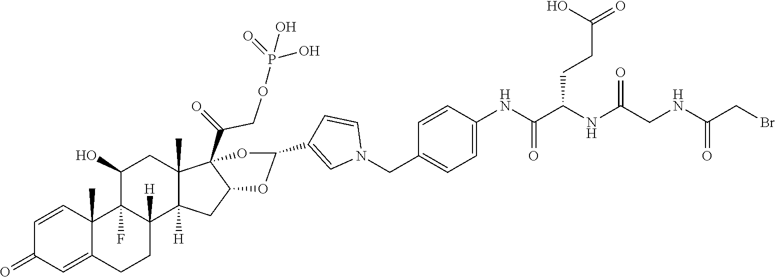 |
| II- D- 29 | 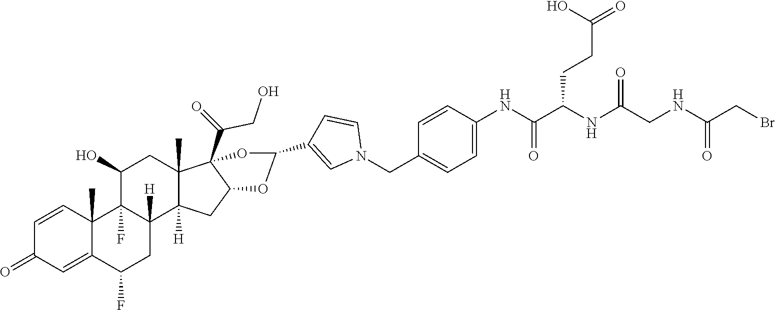 |
| II- D- 30 | 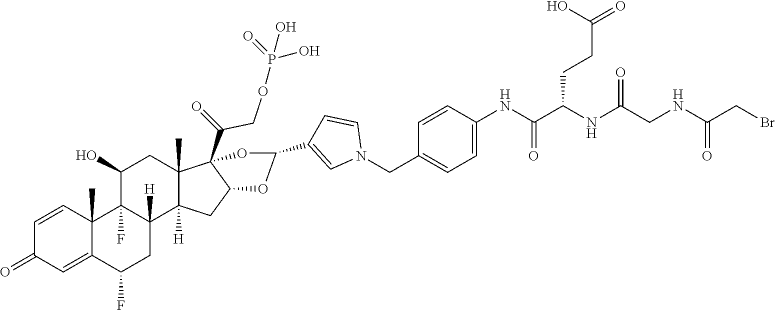 |
| II- D- 36 | 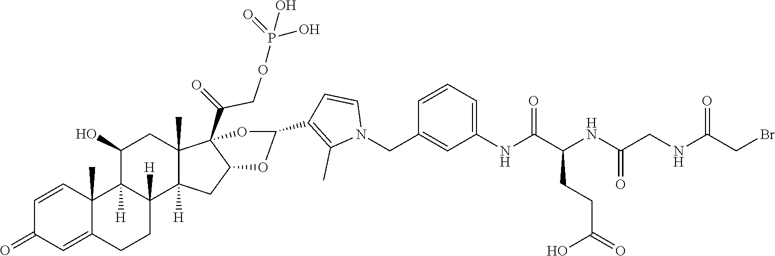 |
| II- D- 39 | 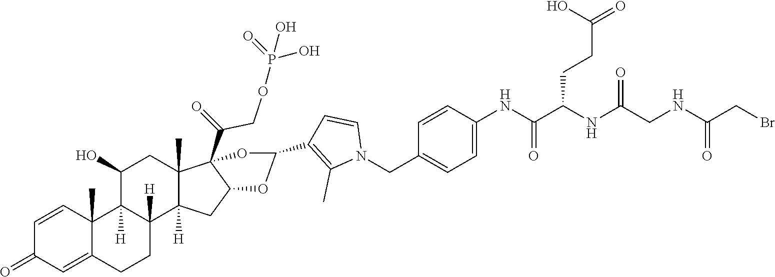 |
| II- D- 42 | 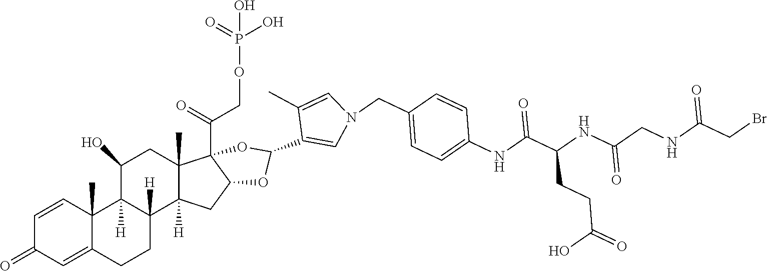 |
| II- D- 45 | 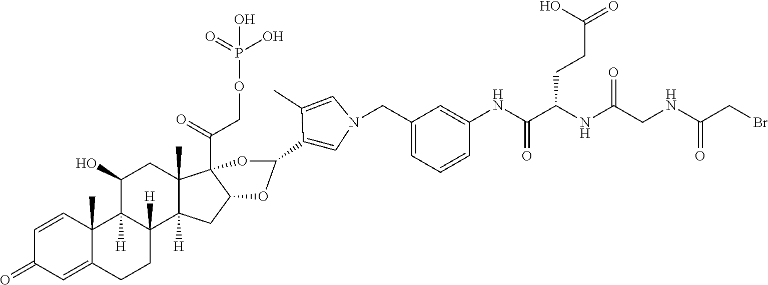 |
| II- D- 48 | 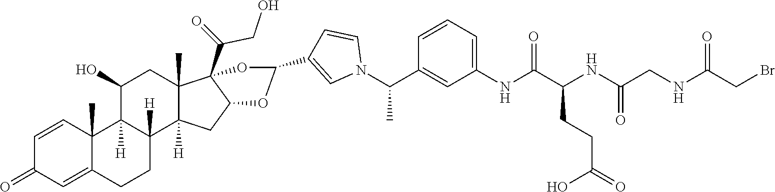 |
| II- D- 49 | 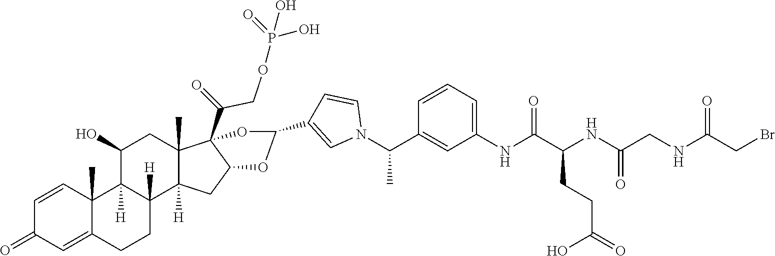 |
| II- D- 53 | 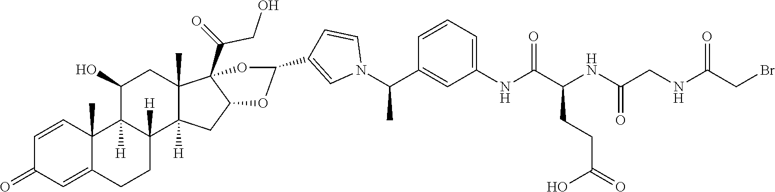 |
| II- D- 54 | 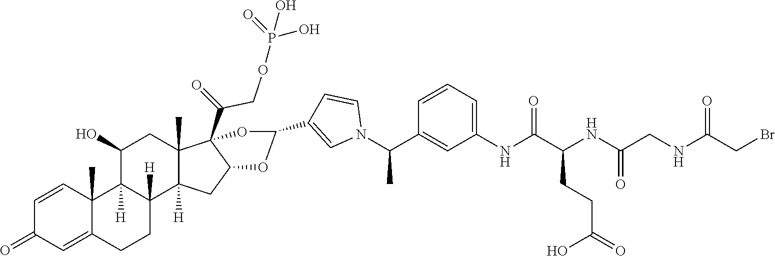 |
| II- D- 78 | 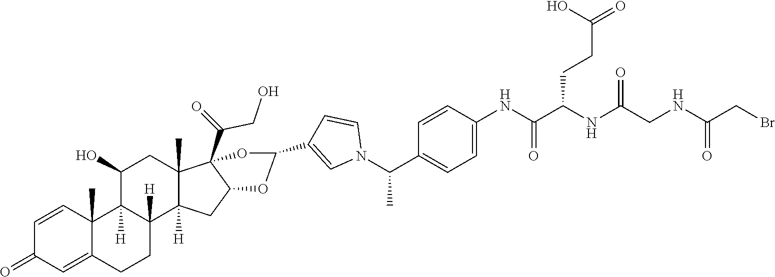 |
| II- D- 79 | 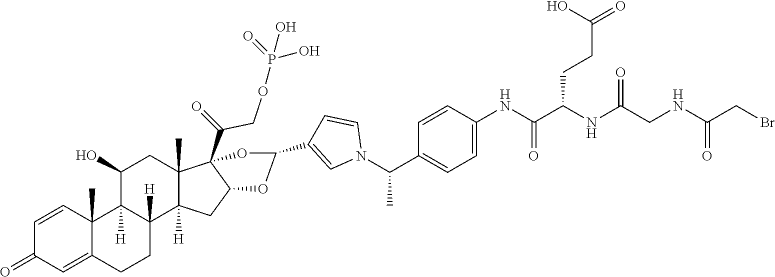 |
| II- D- 92 | 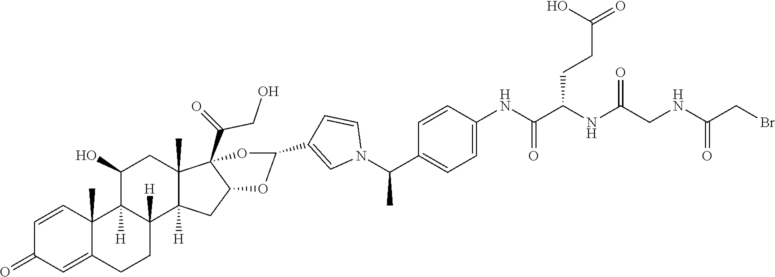 |
| II- D- 93 | 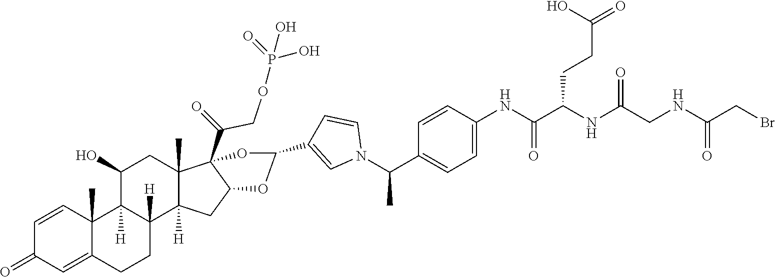 |
| II- D- 111 | 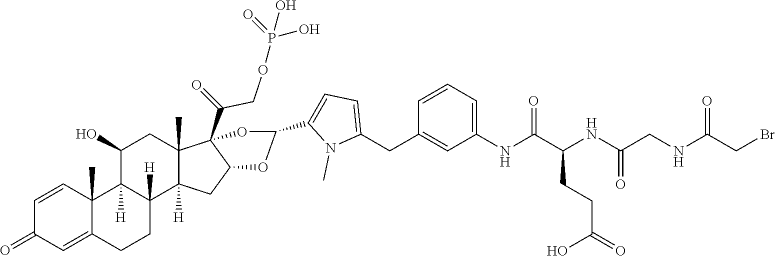 |
| II- D- 114 | 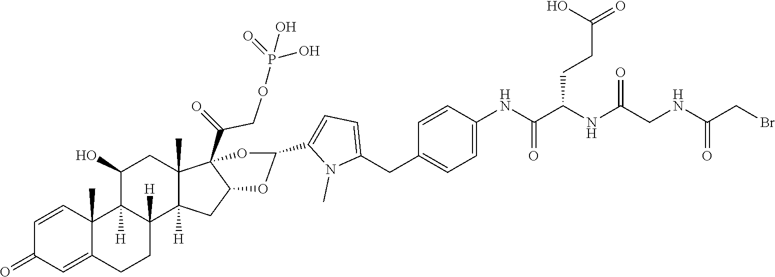 |
| II- D- 117 | 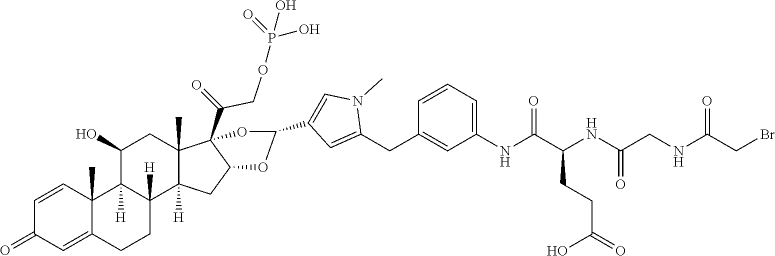 |
| II- D- 120 | 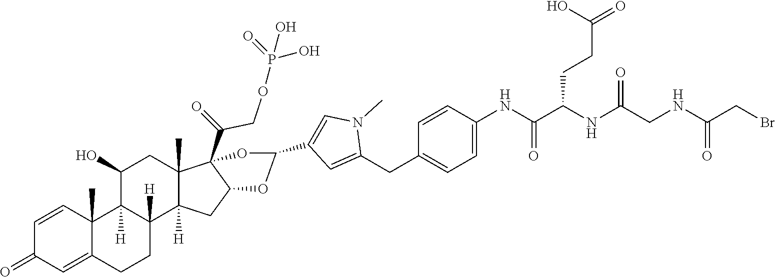 |
| II- D- 123 | 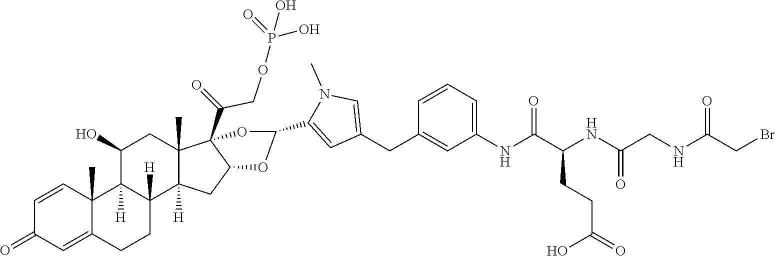 |
| II- D- 126 | 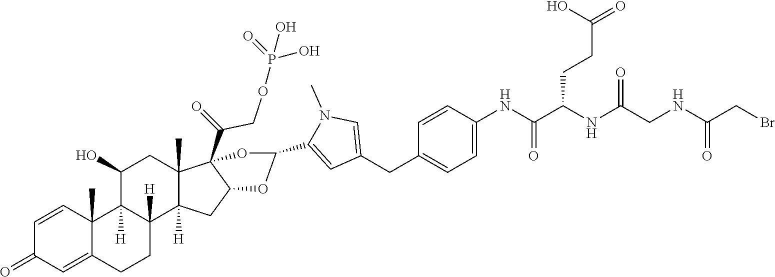 |
| II- D- 170 | 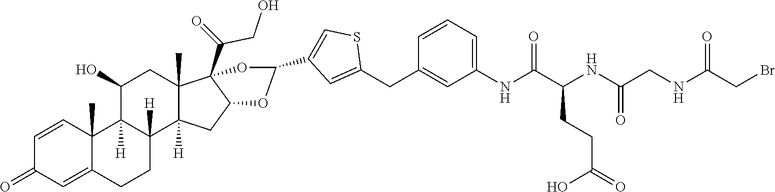 |
| II- D- 171 | 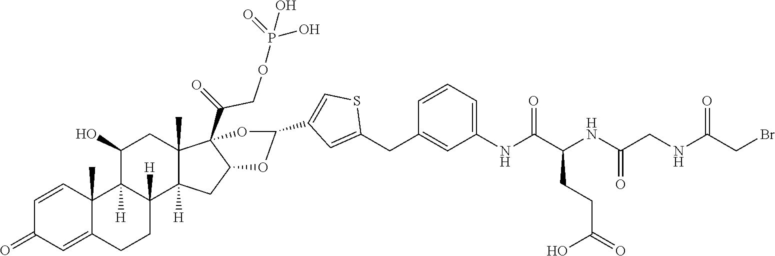 |
| II- D- 175 | 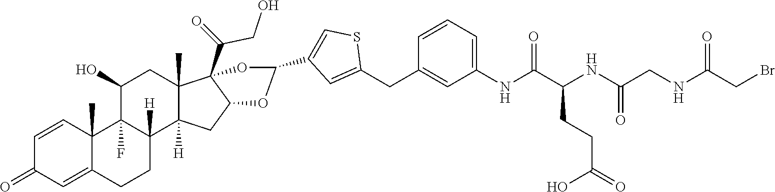 |
| II- D- 176 | 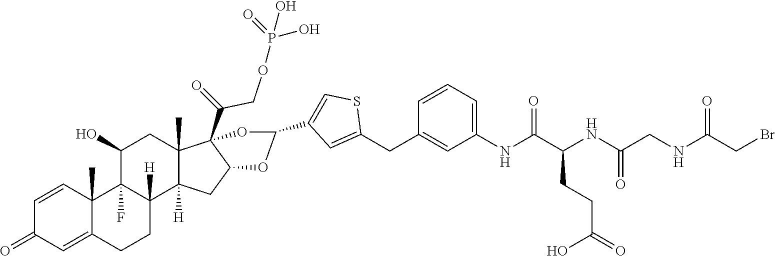 |
| II- D- 180 | 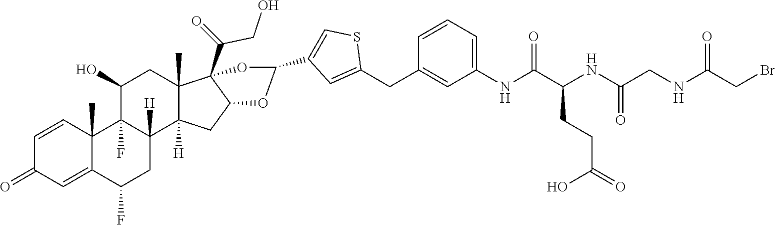 |
| II- D- 181 | 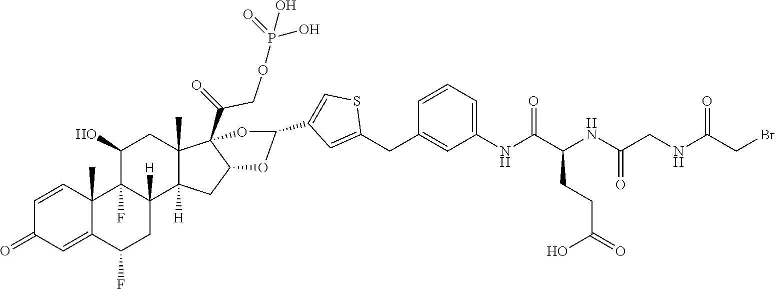 |
| II- D- 185 | 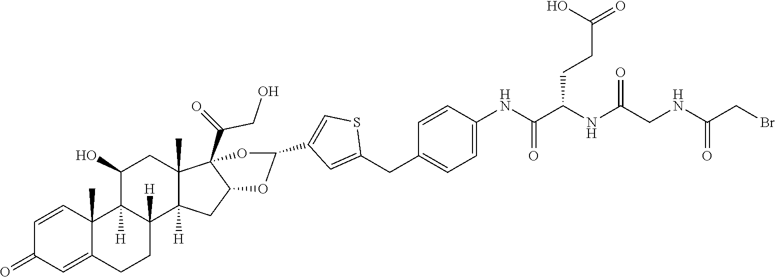 |
| II- D- 186 | 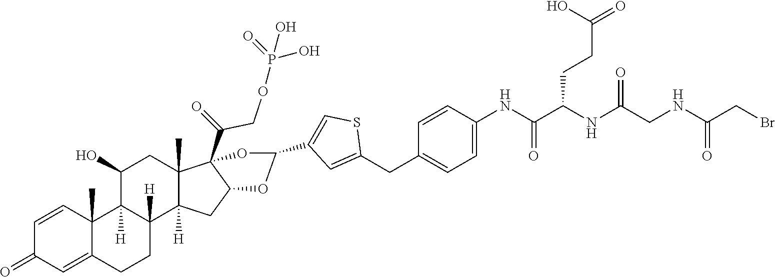 |
| II- D- 190 | 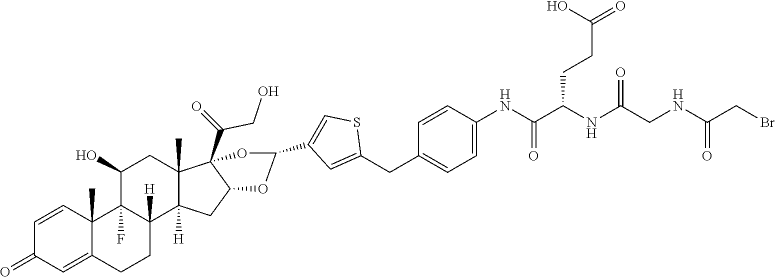 |
| II- D- 191 | 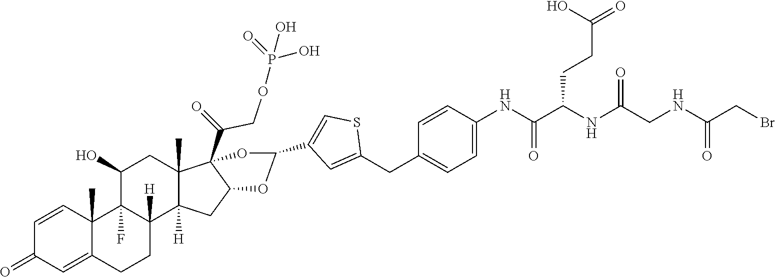 |
| II- D- 195 | 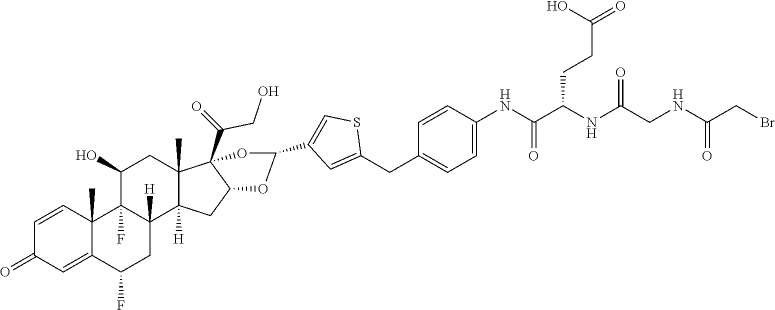 |
| II- D- 196 | 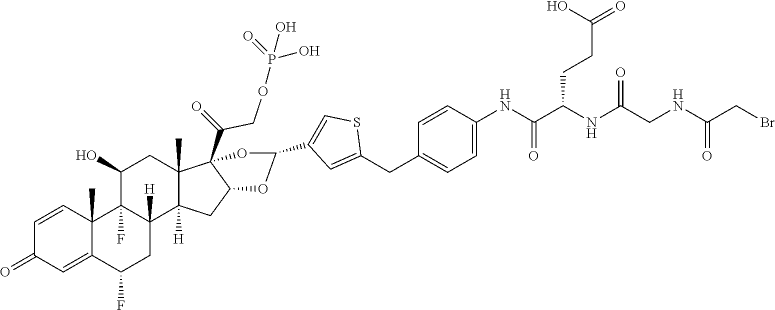 |
| II- D- 200 | 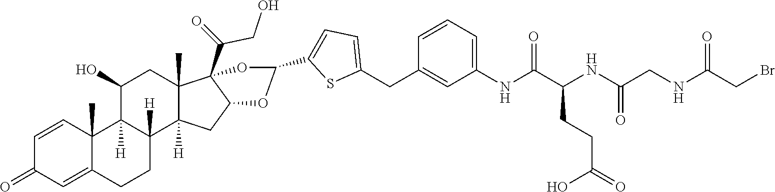 |
| II- D- 201 | 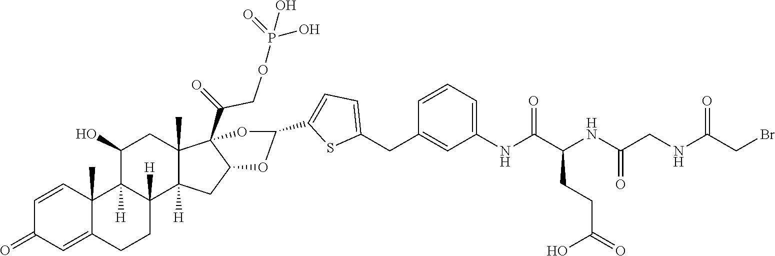 |
| II- D- 205 | 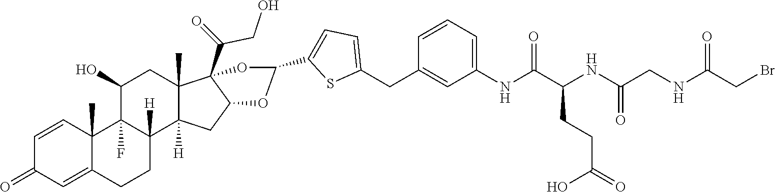 |
| II- D- 206 | 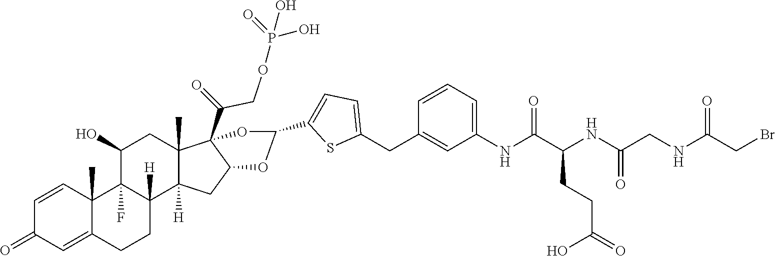 |
| II- D- 210 | 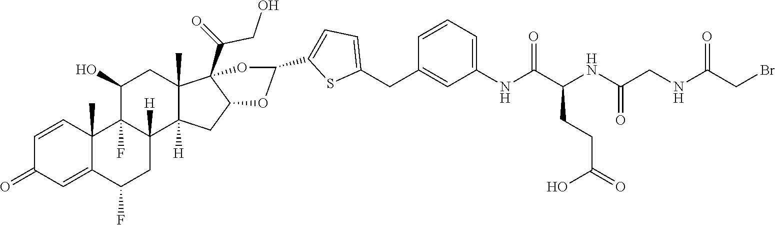 |
| II- D- 211 | 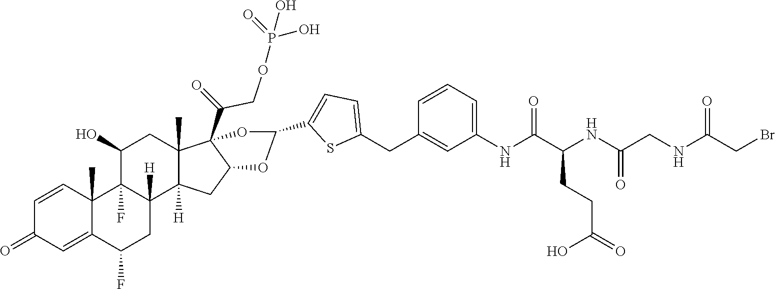 |
| II- D- 215 | 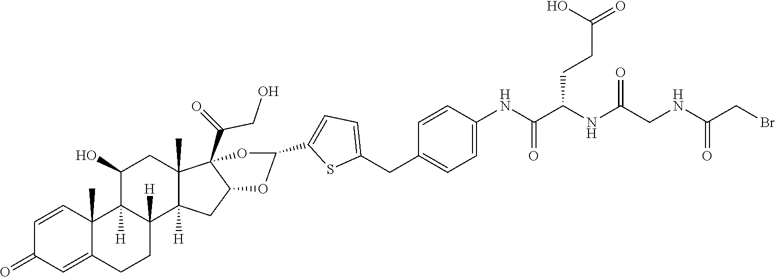 |
| II- D- 216 | 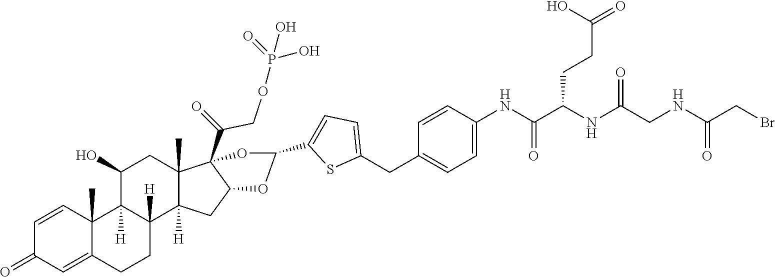 |
| II- D- 220 | 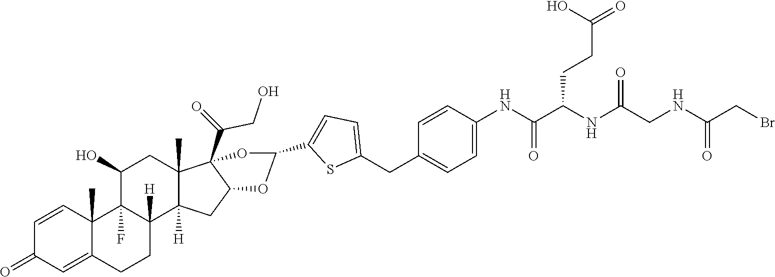 |
| II- D- 221 | 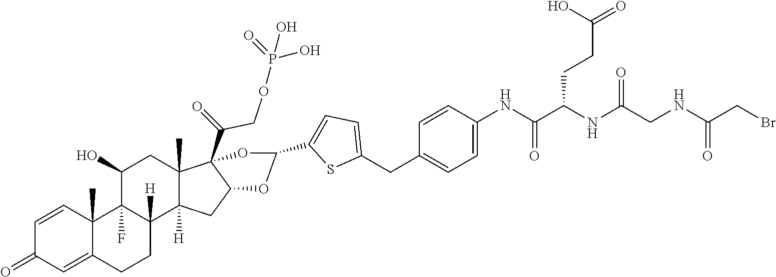 |
| II- D- 225 | 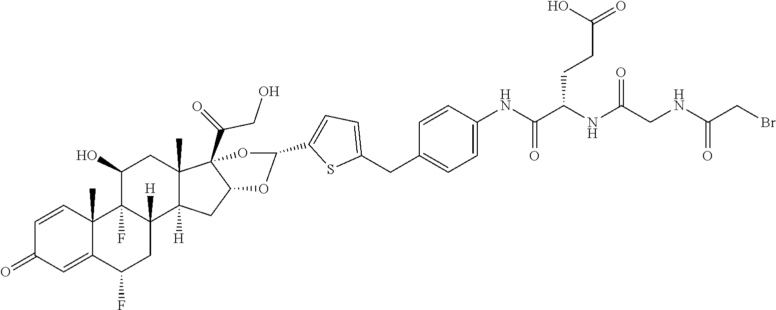 |
| II- D- 226 | 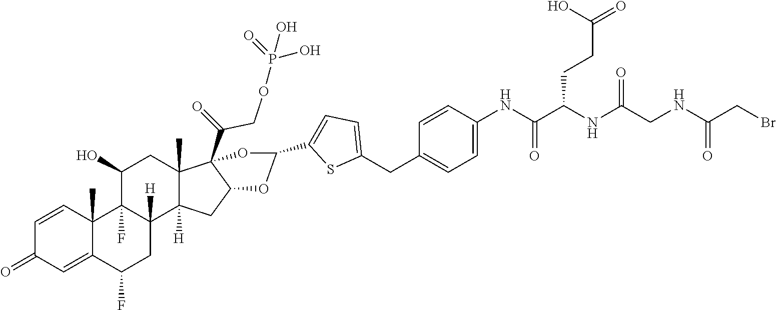 |
| II- D- 238 | 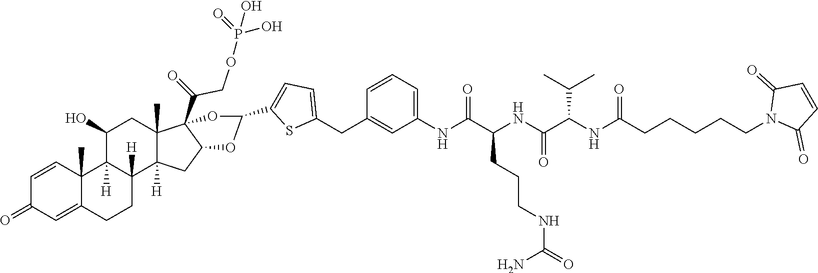 |
| II- D- 239 | 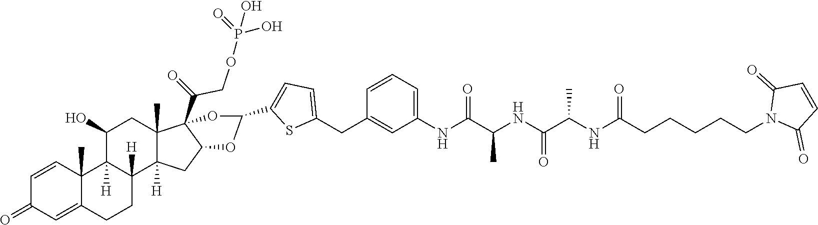 |
| II- D- 240 | 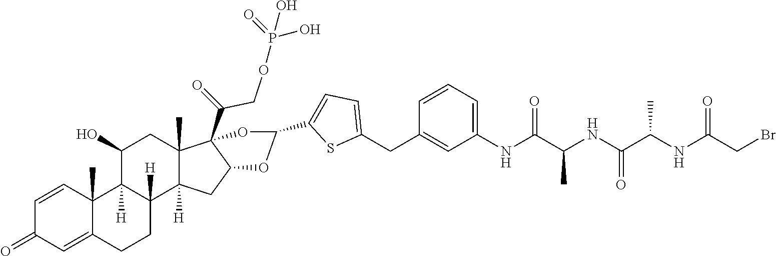 |
| II- D- 241 | 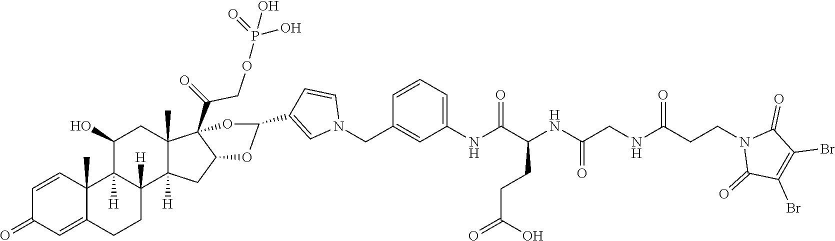 |
| II- D- 244 | 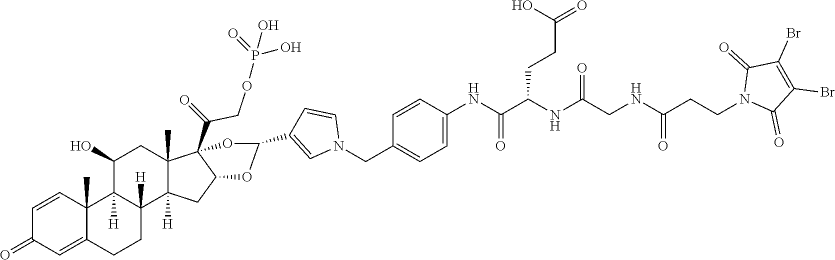 |
| II- D- 247 | 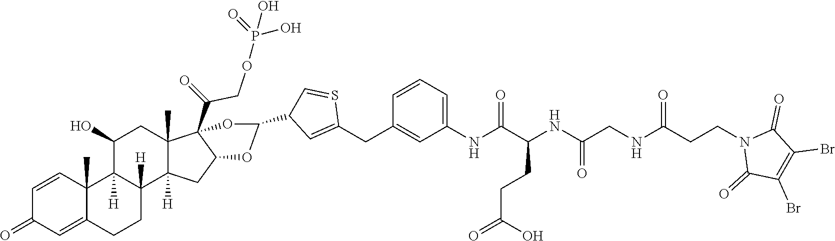 |
| II- D- 248 | 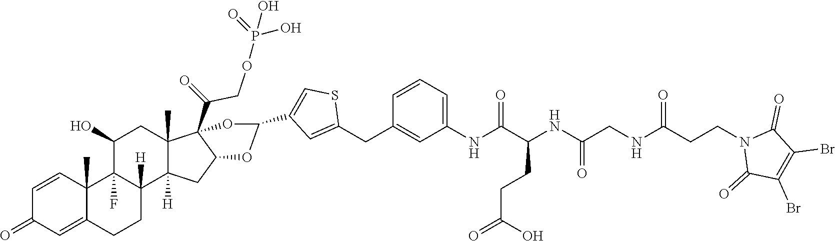 |
| II- D- 249 | 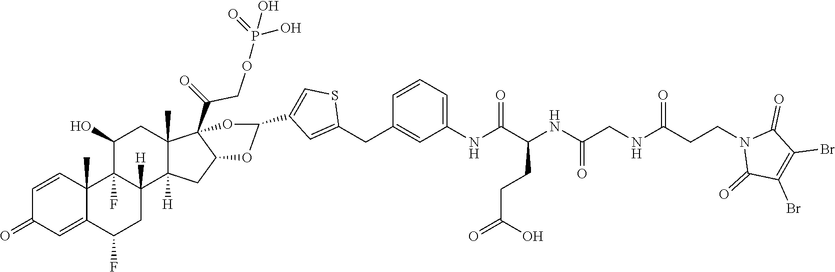 |
| II- D- 250 | 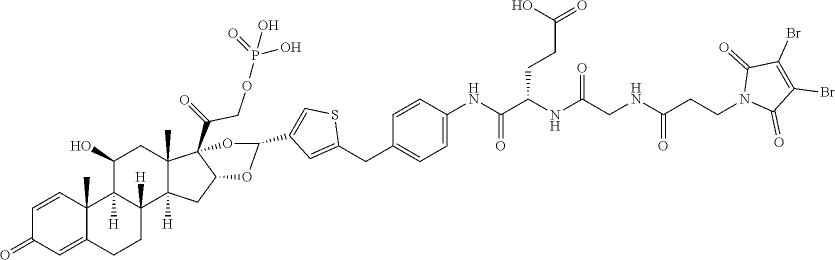 |
| II- D- 251 | 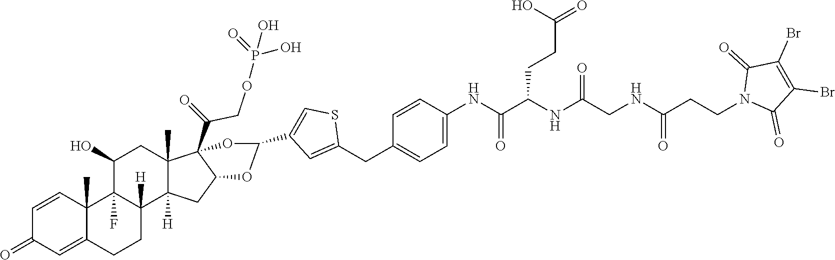 |
| II- D- 252 | 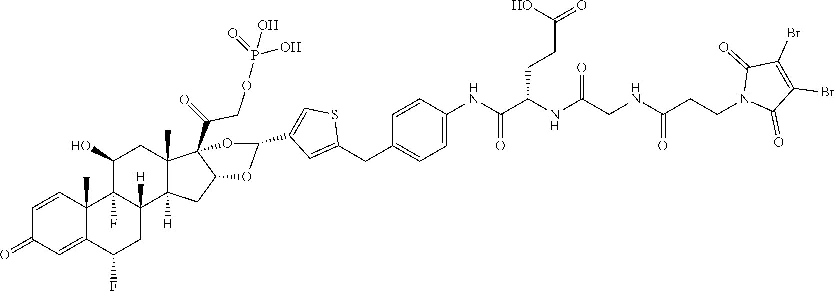 |
| II- D- 253 | 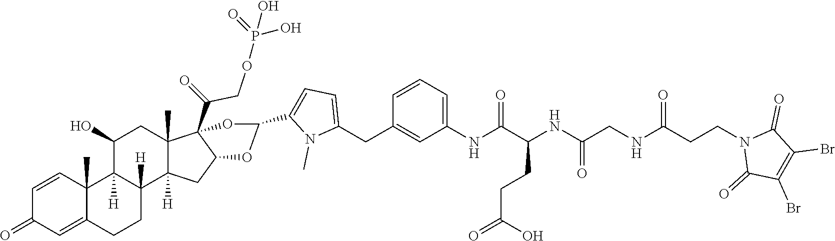 |
| II- D- 256 | 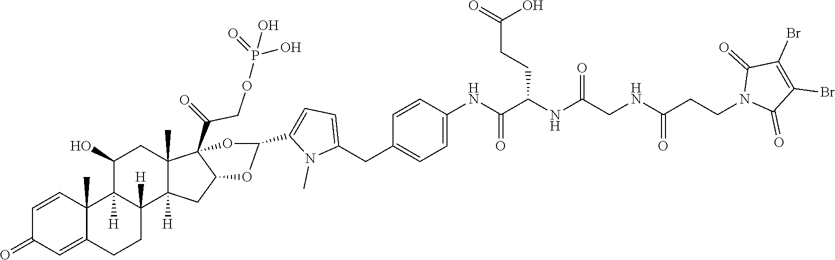 |
| II- D- 259 | 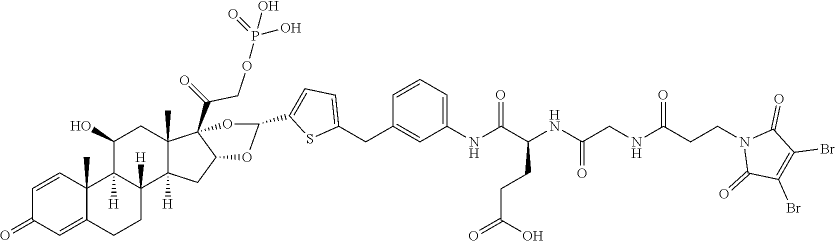 |
| II- D- 260 | 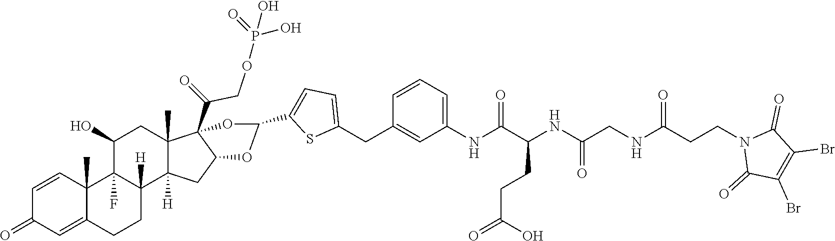 |
| II- D- 261 | 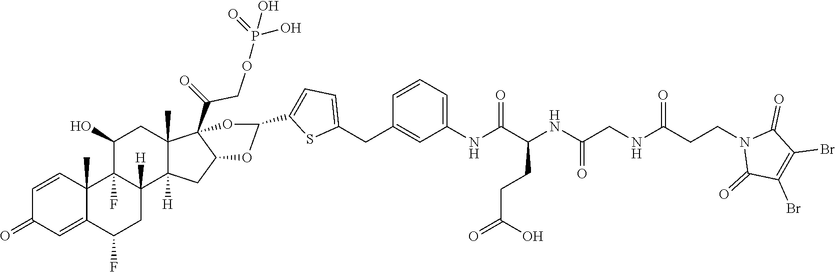 |
| II- D- 262 | 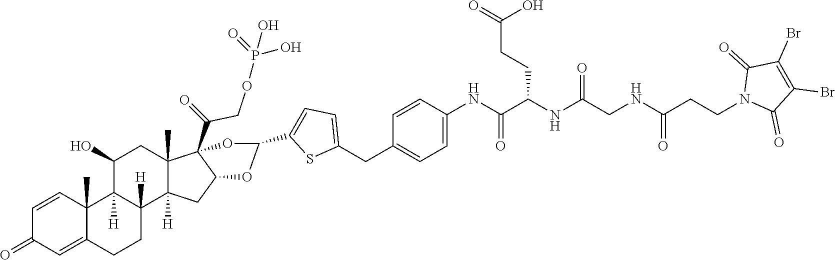 |
| II- D- 263 | 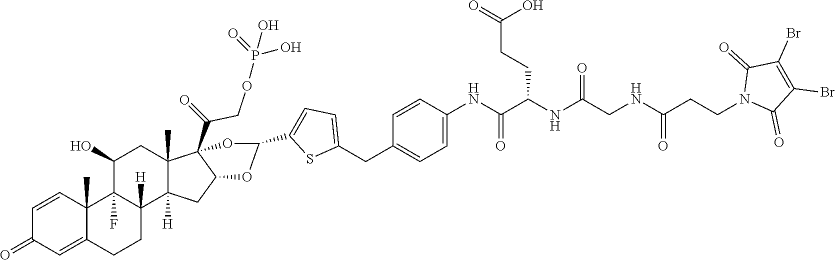 |
| II- D- 264 | 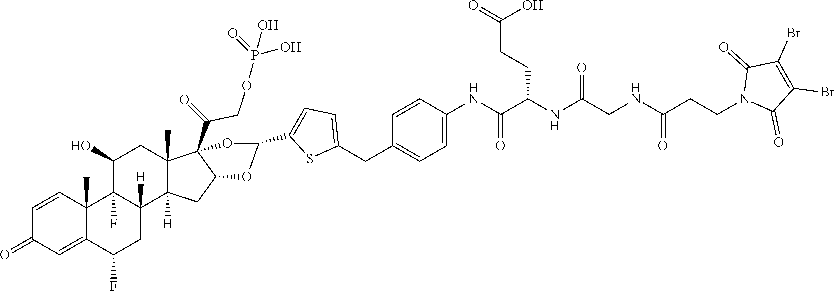 |
| II- D- 265 | 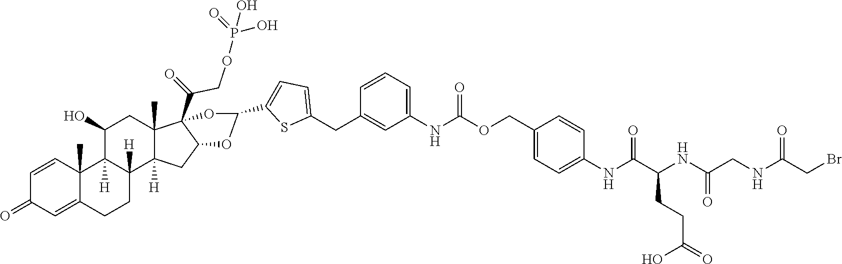 |
| II- D- 266 | 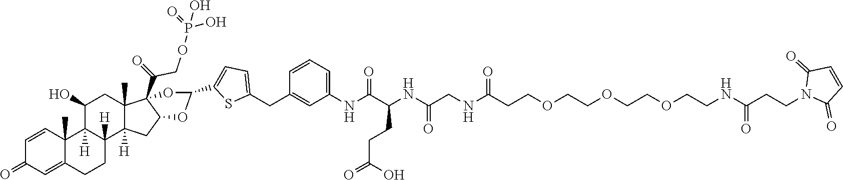 |
| II- D- 267 | 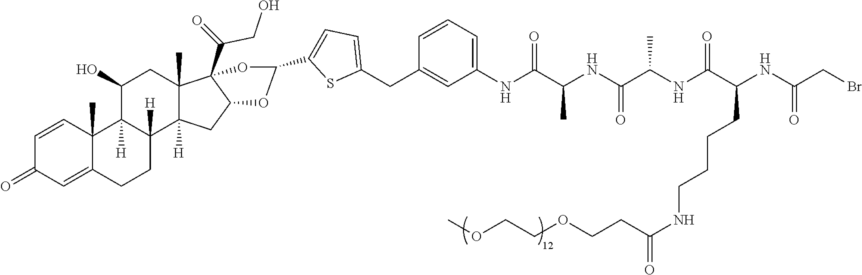 |
| II- D- 268 | 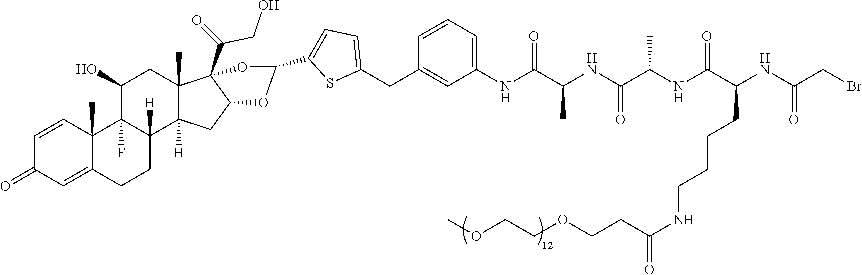 |
| II- D- 269 | 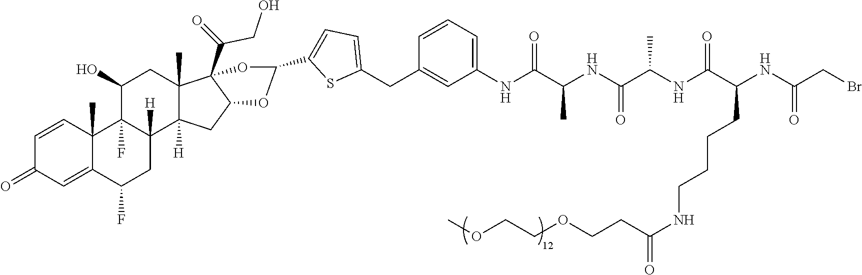 |
| II- D- 270 | 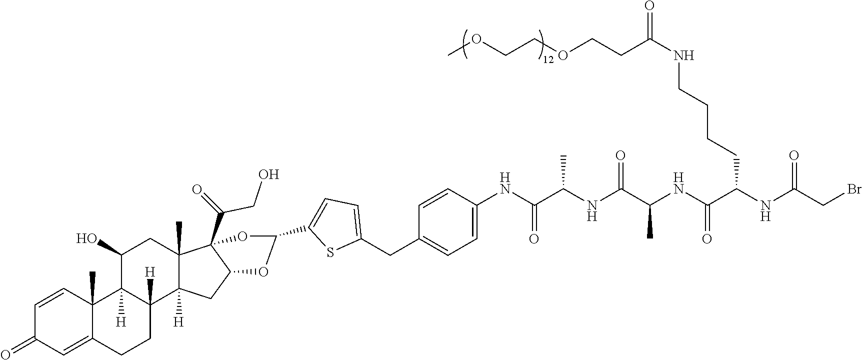 |
| II- D- 271 | 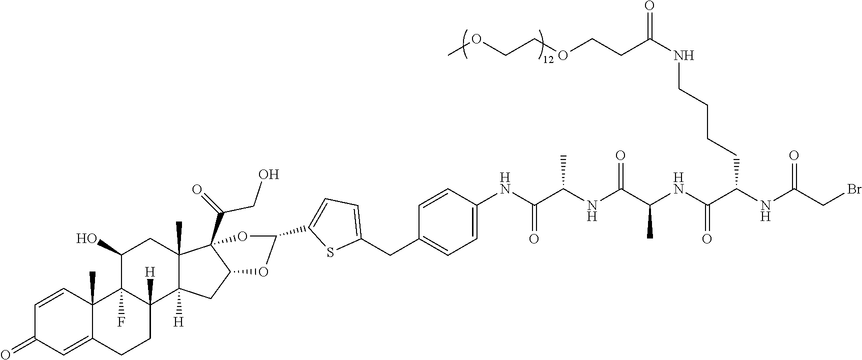 |
| II- D- 272 | 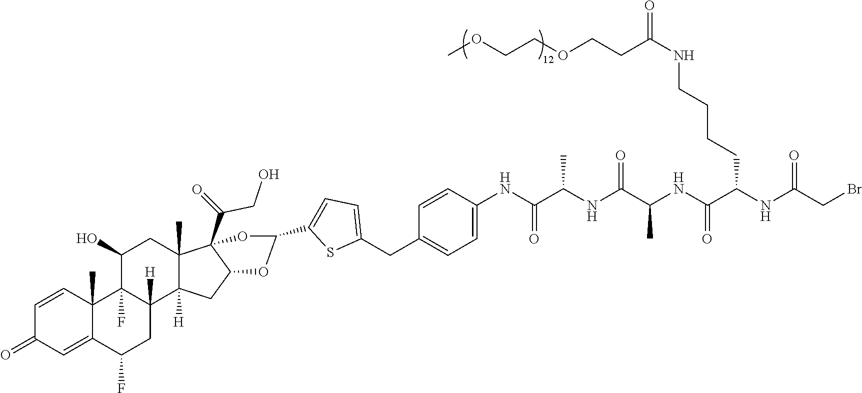 |
| II- D- 273 | 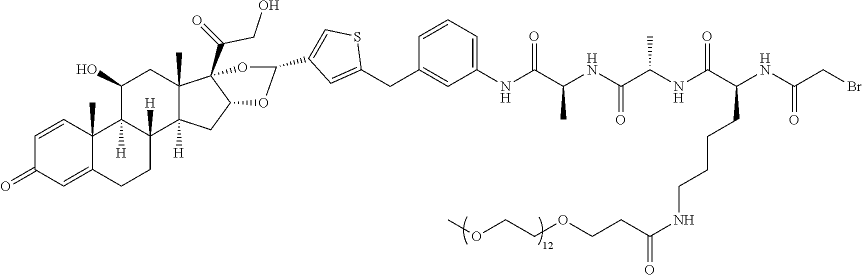 |
| II- D- 274 | 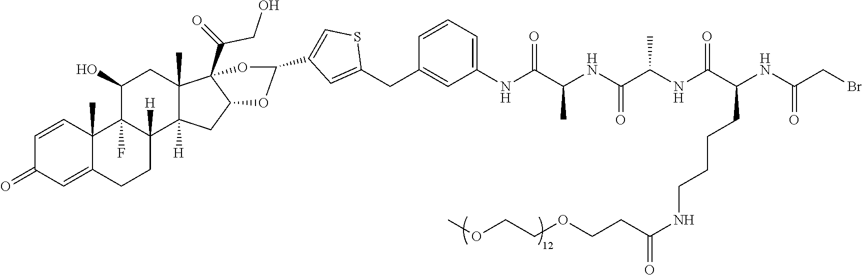 |
| II- D- 275 | 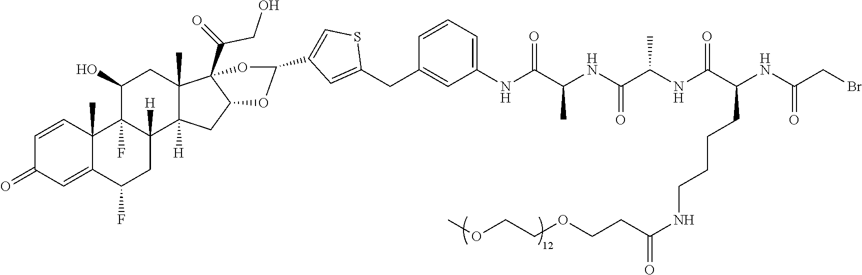 |
| II- D- 276 | 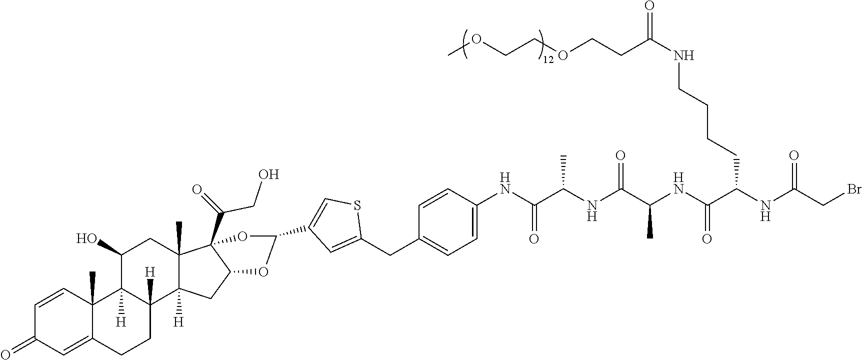 |
| II- D- 277 | 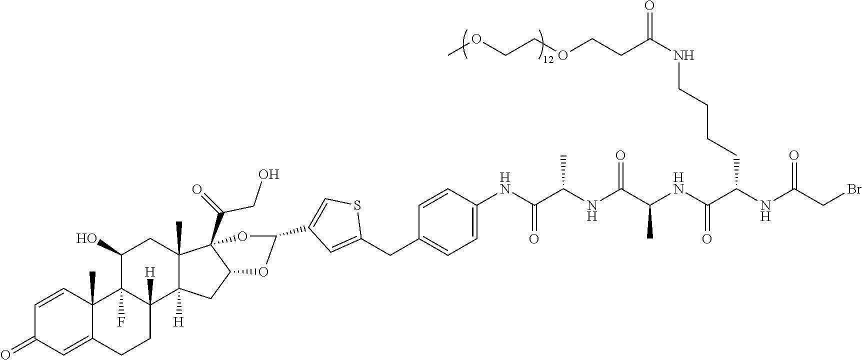 |
| II- D- 278 | 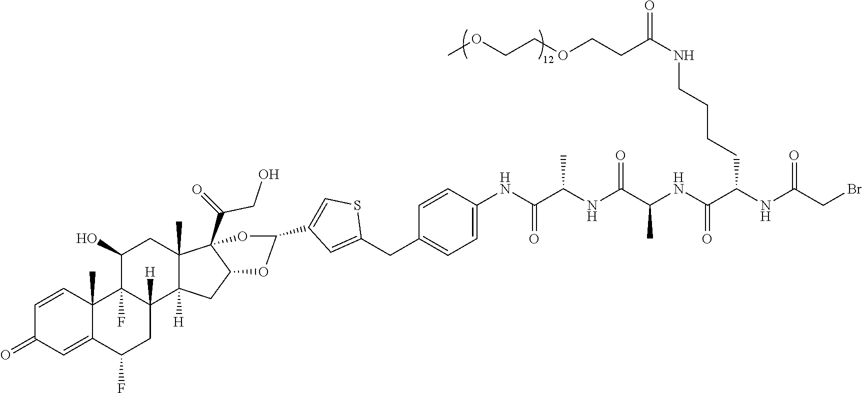 |
| II- D- 279 | 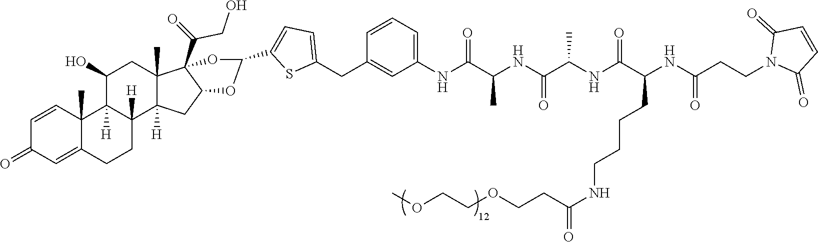 |
| II- D- 280 | 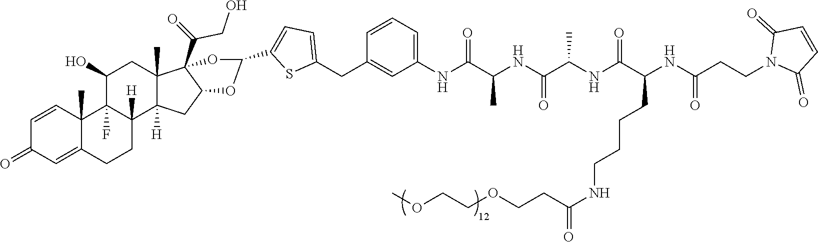 |
| II- D- 281 | 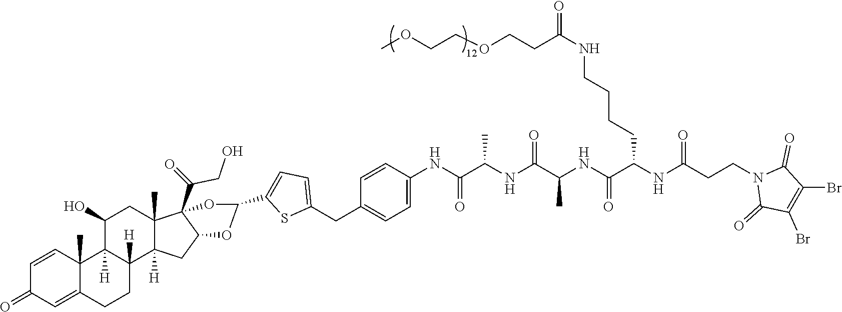 |
| II- D- 282 | 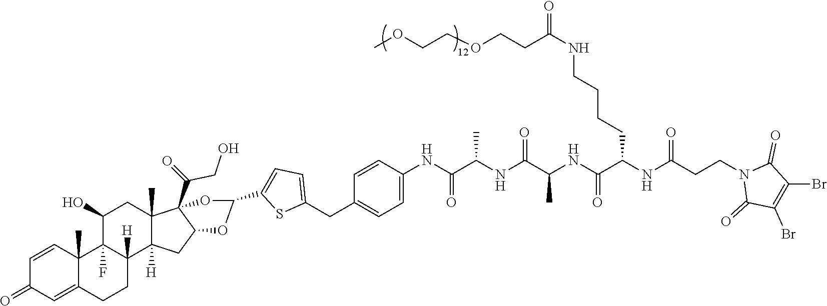 |
| II- D- 283 | 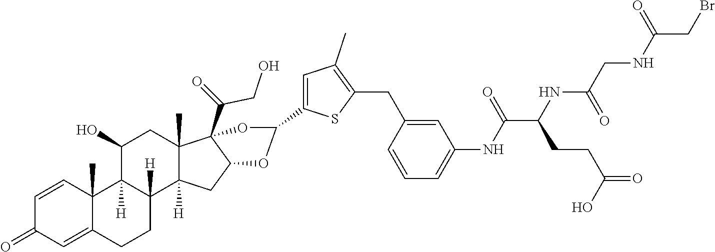 |
| II- D- 286 | 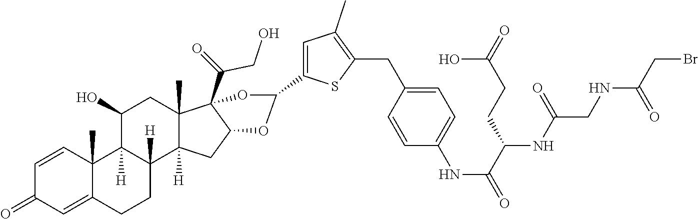 |
| II- D- 289 | 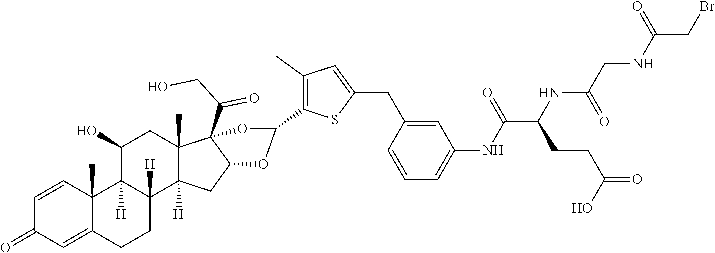 |
| II- D- 292 | 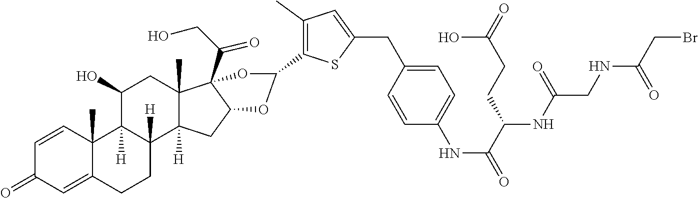 |
| II- D- 295 | 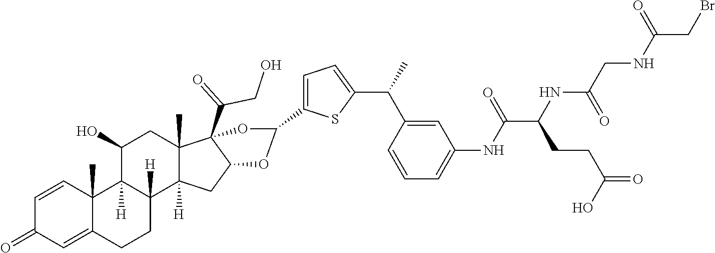 |
| II- D- 298 | 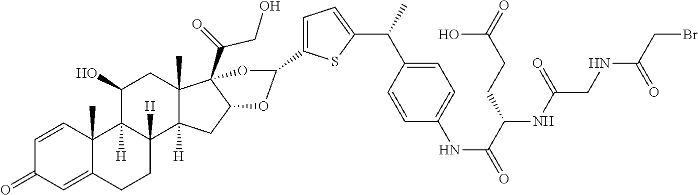 |
| II- D- 301 | 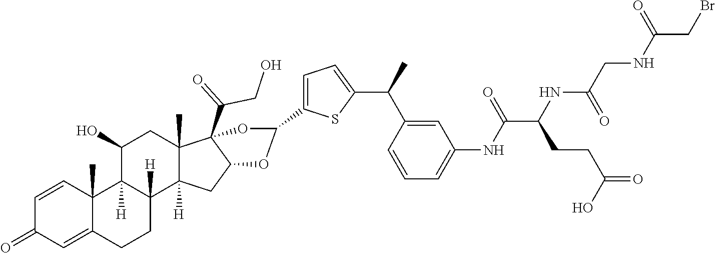 |
| II- D- 304 | 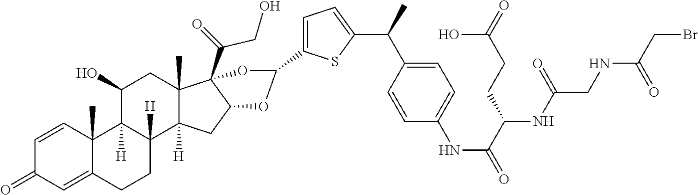 |
| II- D- 307 | 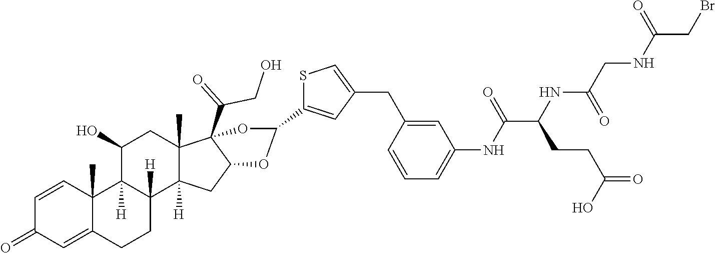 |
| II- D- 310 | 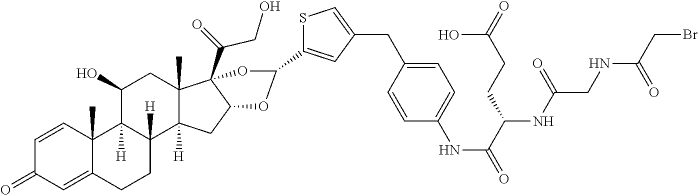 |
| II- D- 313 | 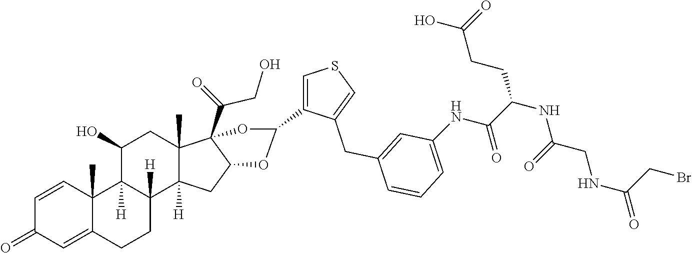 |
| II- D- 314 | 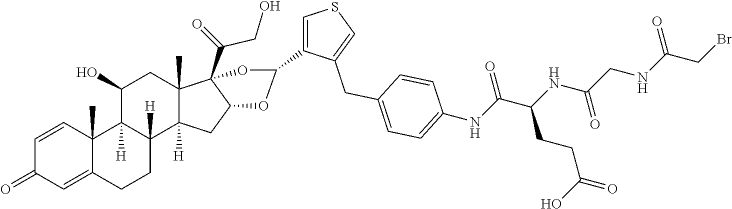 |
| II- D- 315 | 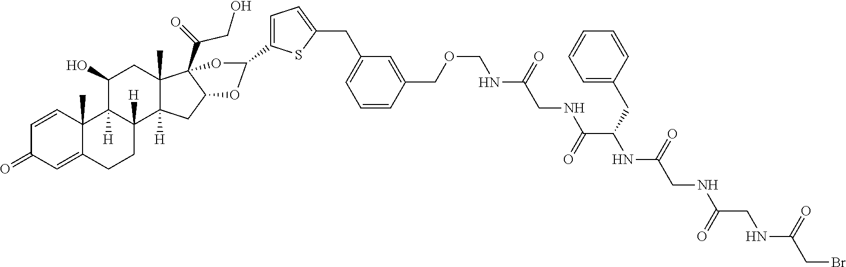 |
| II- D- 316 | 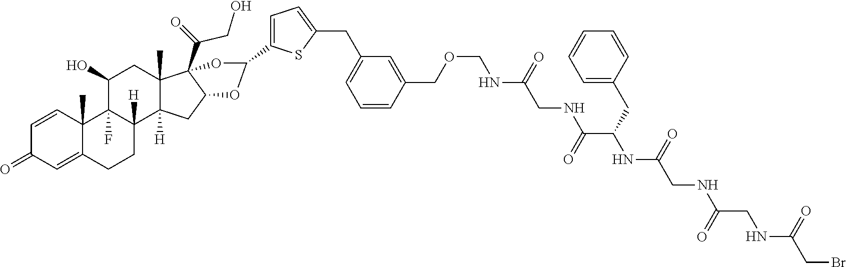 |
| II- D- 317 | 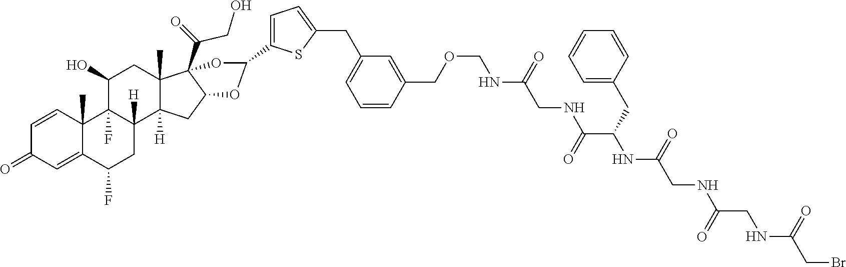 |
| II- D- 318 | 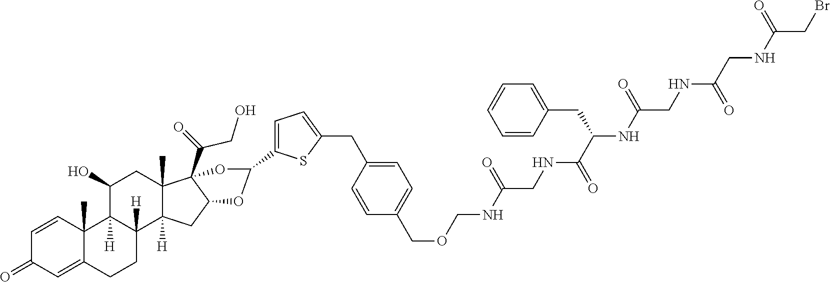 |
| II- D- 319 | 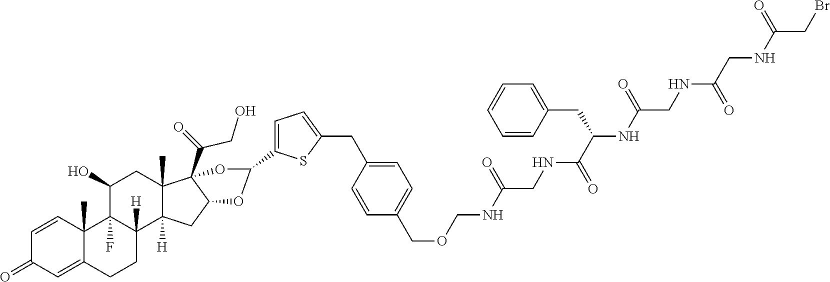 |
| II- D- 320 | 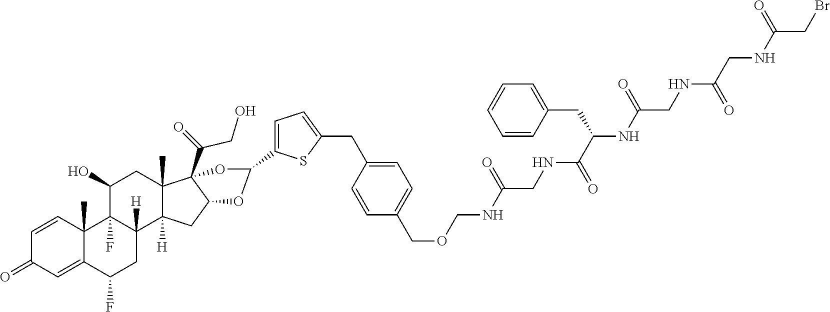 |
| II- D- 321 | 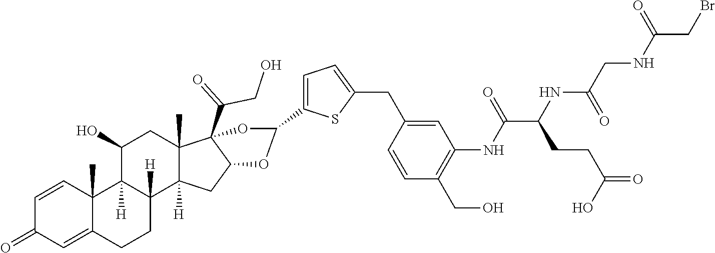 |
| II- D- 322 | 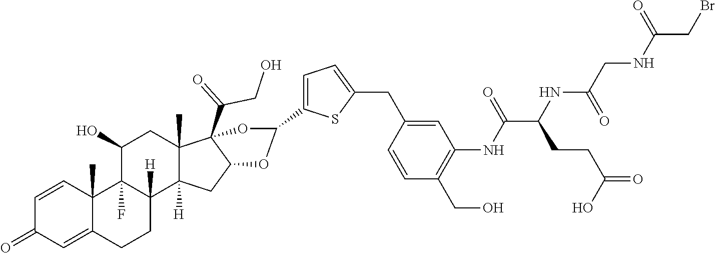 |
| II- D- 323 | 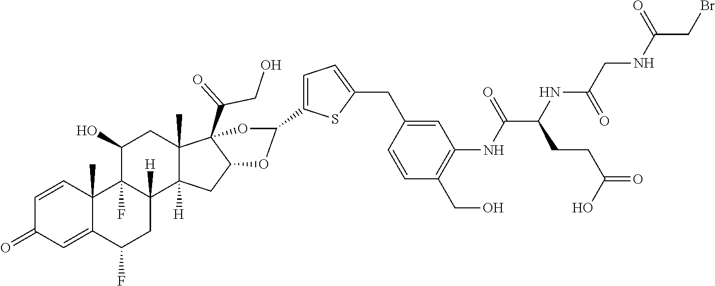 |
| II- D- 324 | 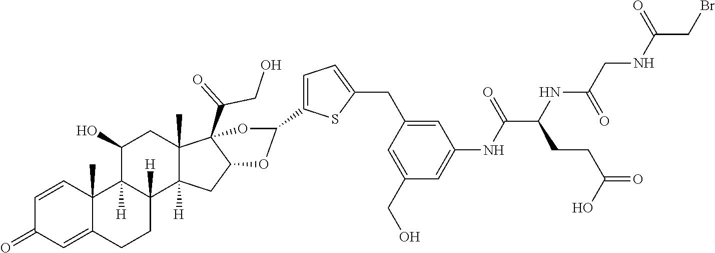 |
| II- D- 325 | 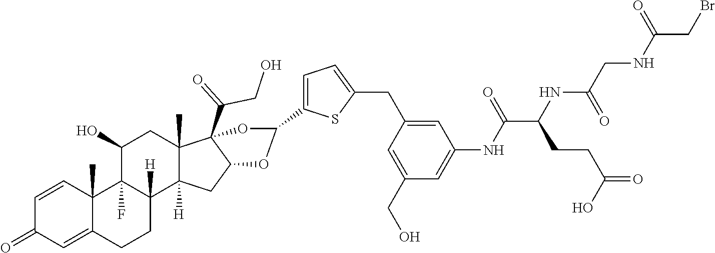 |
| II- D- 326 | 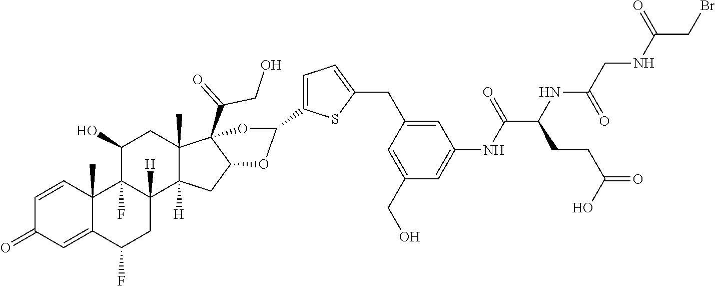 |
| II- D- 327 | 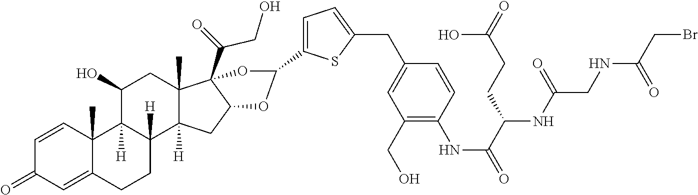 |
| II- D- 328 | 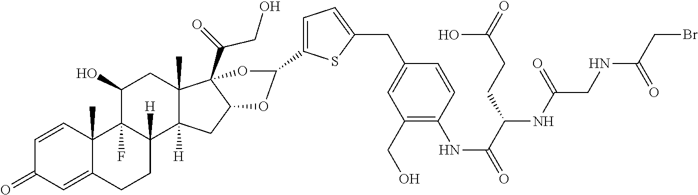 |
| II- D- 329 | 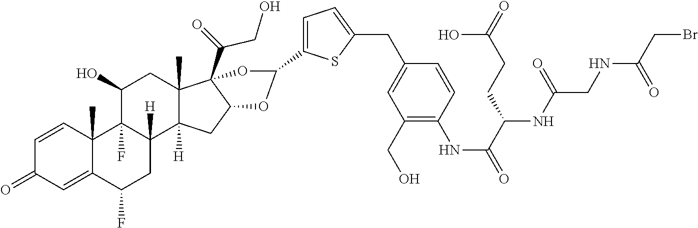 |
| II- D- 330 | 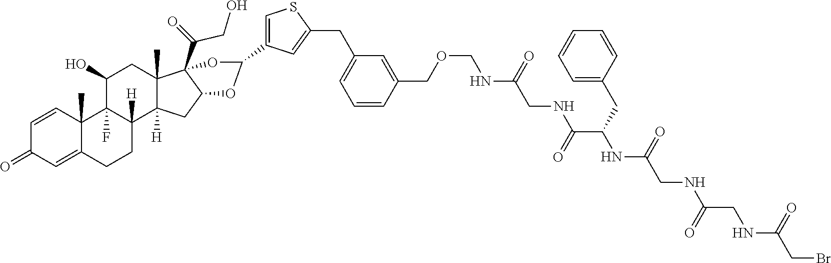 |
| II- D- 331 | 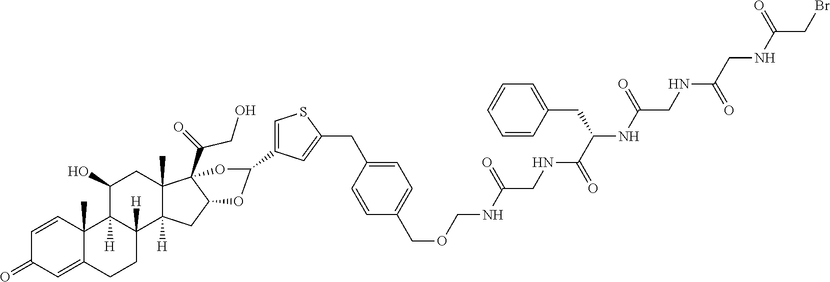 |
| II- D- 332 | 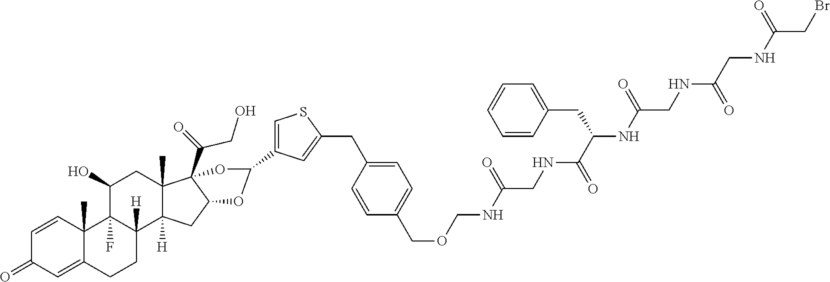 |
| II- D- 333 | 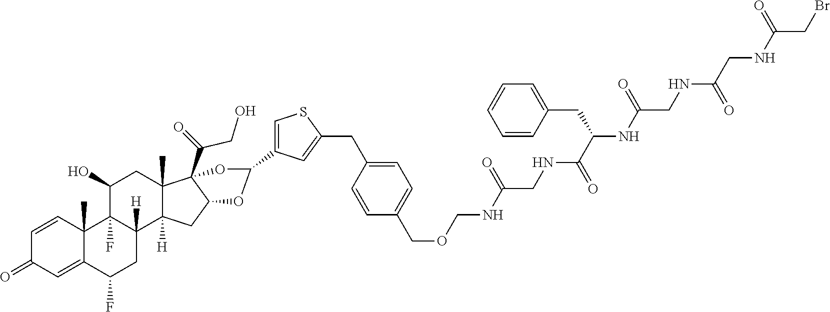 |
| II- D- 334 | 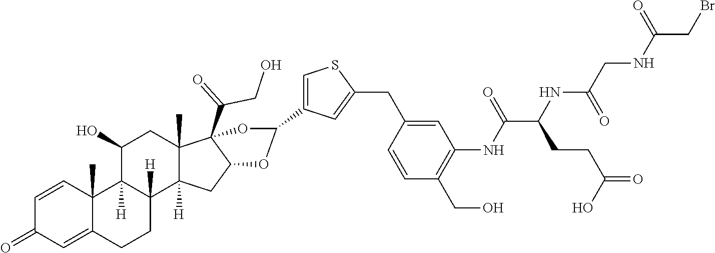 |
| II- D- 335 | 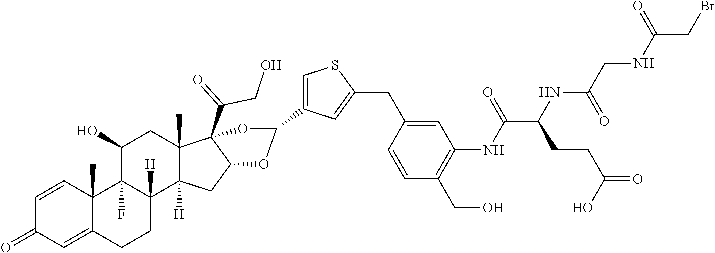 |
| II- D- 336 | 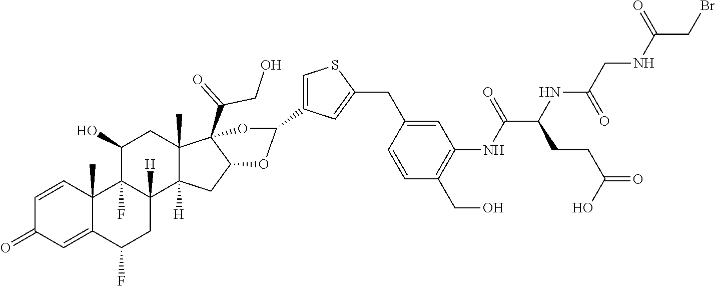 |
| II- D- 337 | 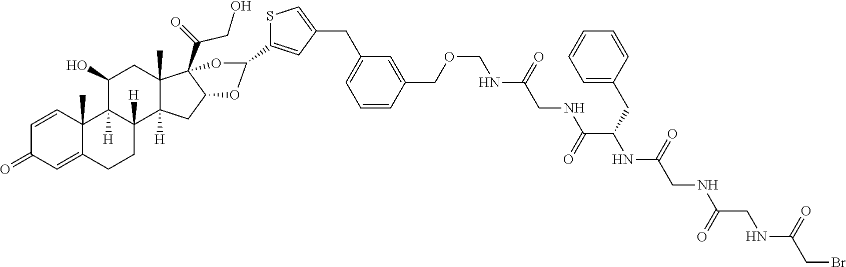 |
| II- D- 338 | 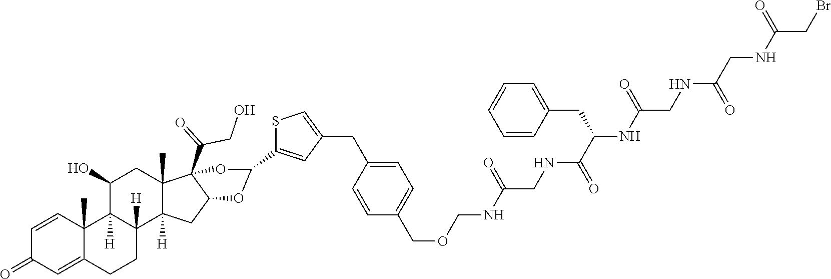 |
| II- D- 339 | 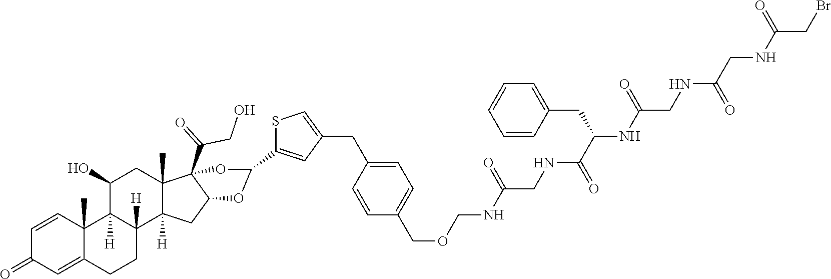 |
| II- D- 340 | 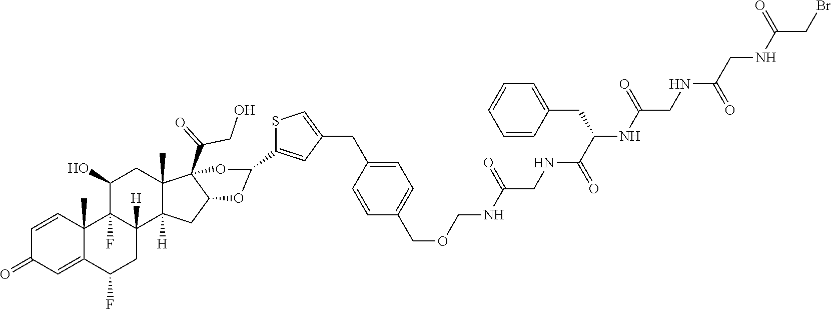 |
| II- D- 341 | 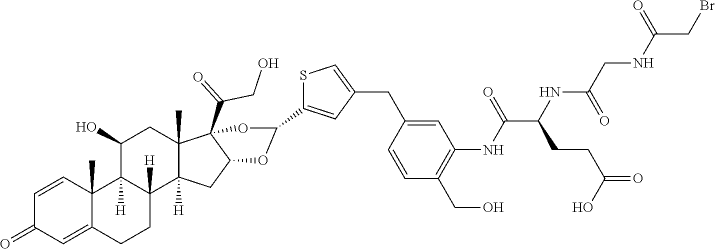 |
| II- D- 342 | 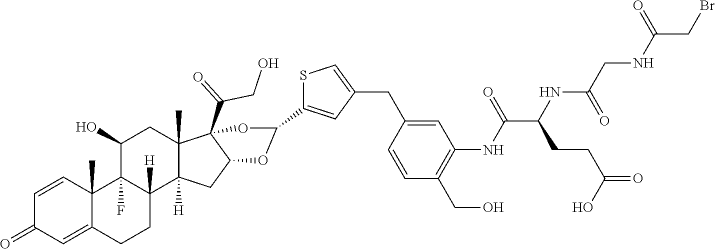 |
| II- D- 343 | 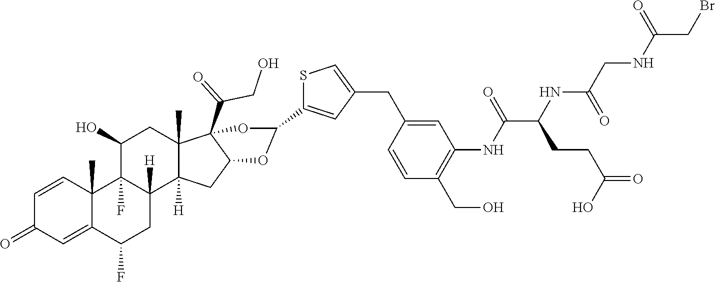 |
| II- D- 344 | 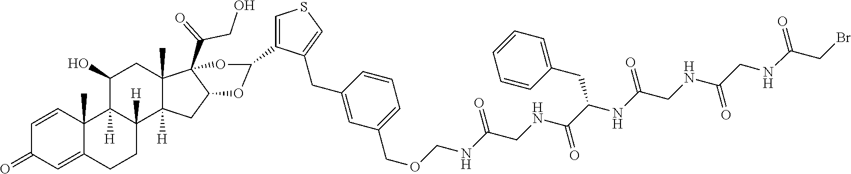 |
| II- D- 345 | 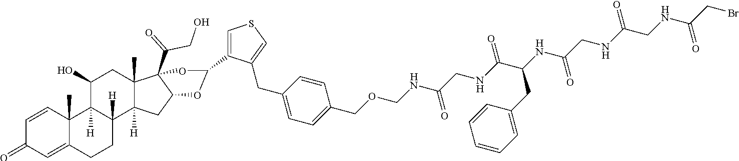 |
| II- D- 361 | 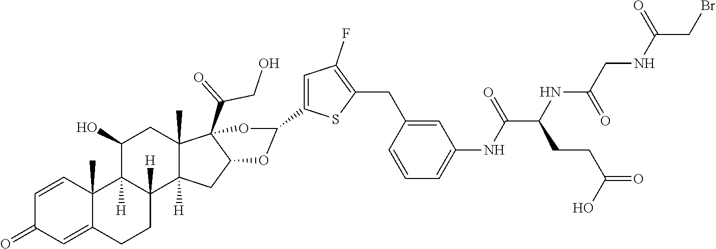 |
| II- D- 364 | 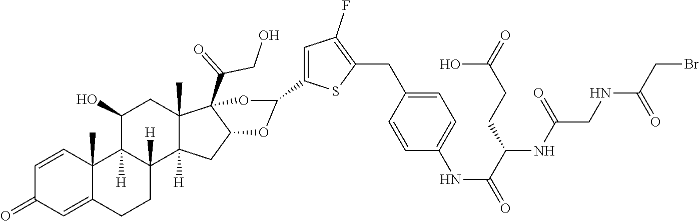 |
| II- D- 367 | 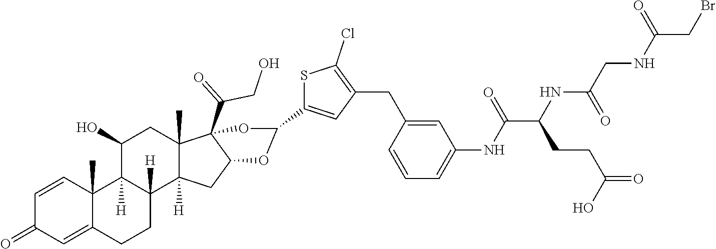 |
| II- D- 370 | 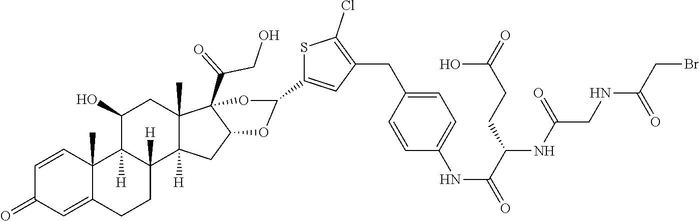 |
| II- D- 373 | 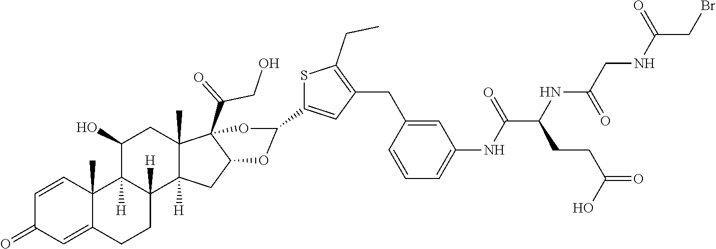 |
| II- D- 376 | 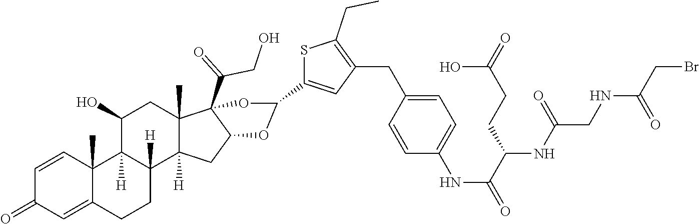 |
| II- D- 379 | 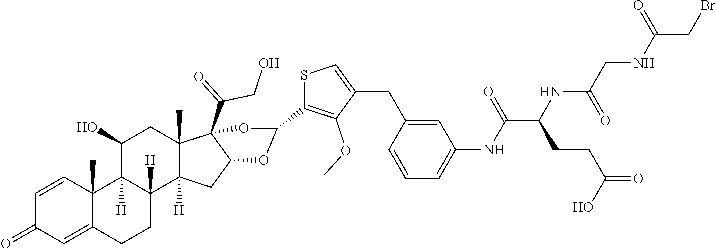 |
| II- D- 382 | 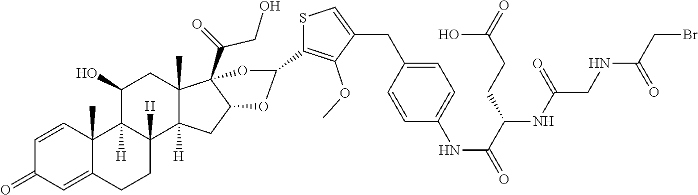 |
| II- D- 385 | 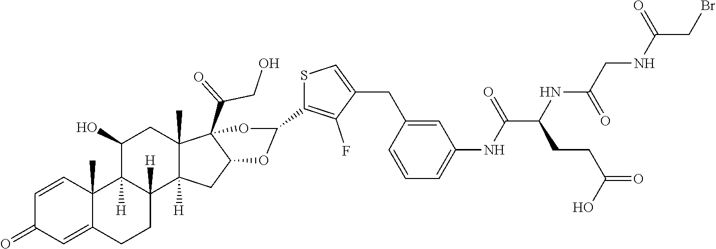 |
| II- D- 388 | 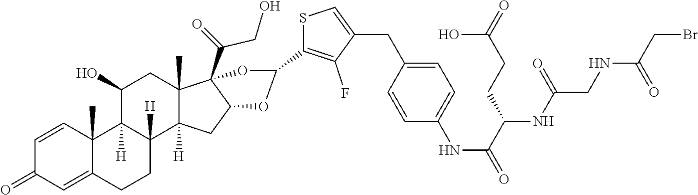 |
| II- D- 391 | 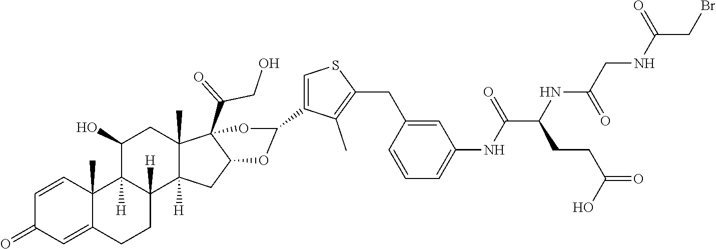 |
| II- D- 394 | 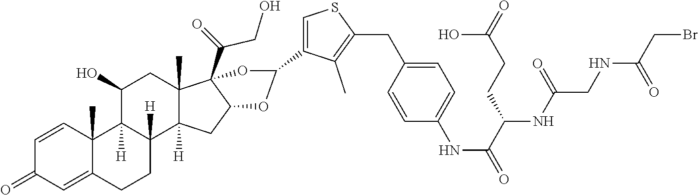 |
| II- D- 397 | 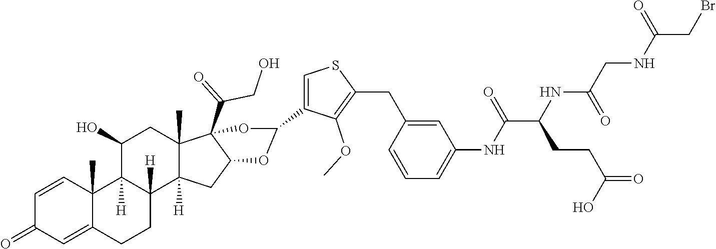 |
| II- D- 400 | 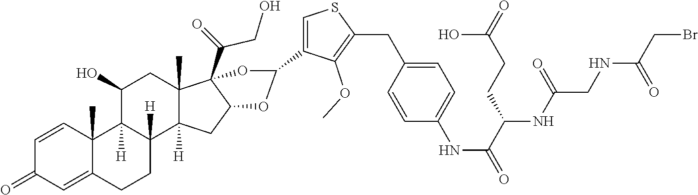 |
| II- D- 403 | 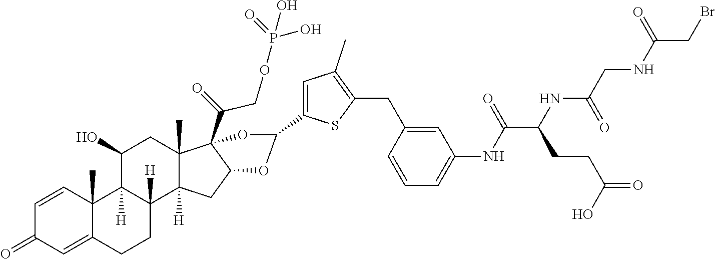 |
| II- D- 406 | 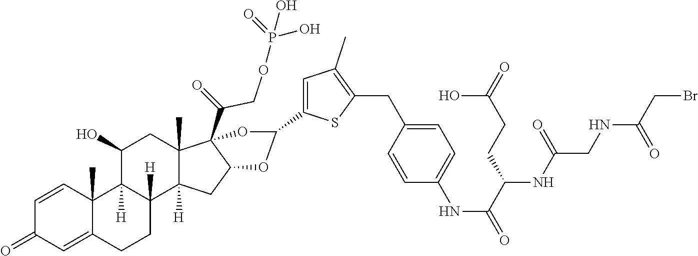 |
| II- D- 409 | 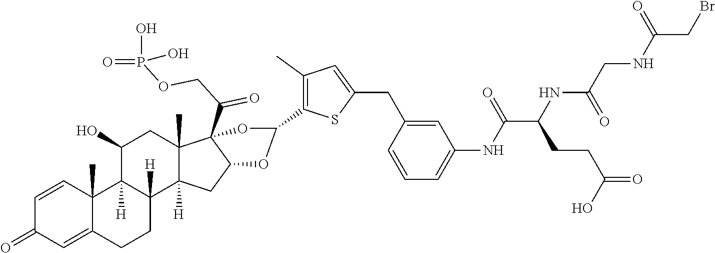 |
| II- D- 412 | 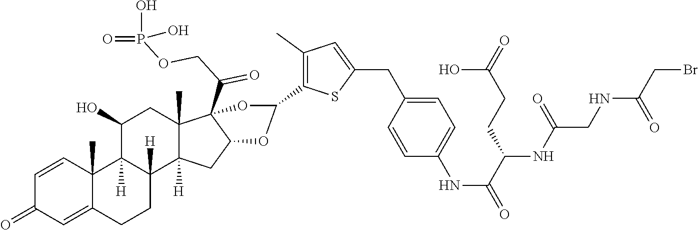 |
| II- D- 415 | 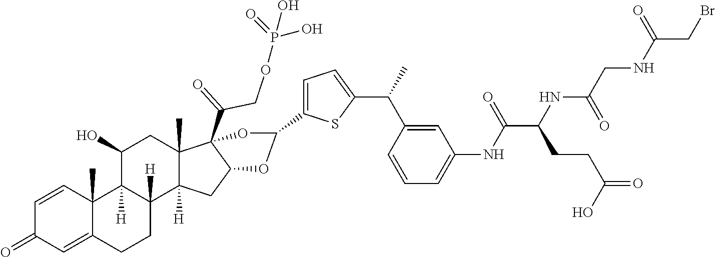 |
| II- D- 418 | 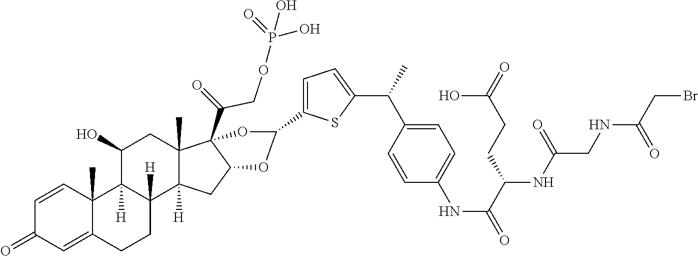 |
| II- D- 421 | 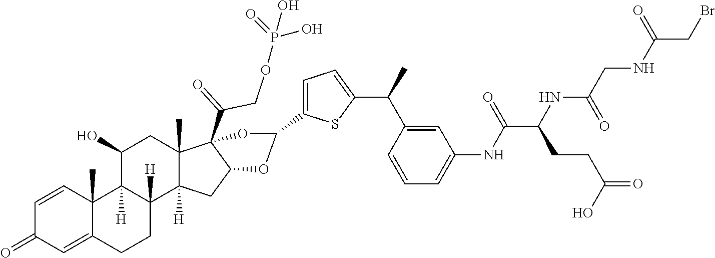 |
| II- D- 424 | 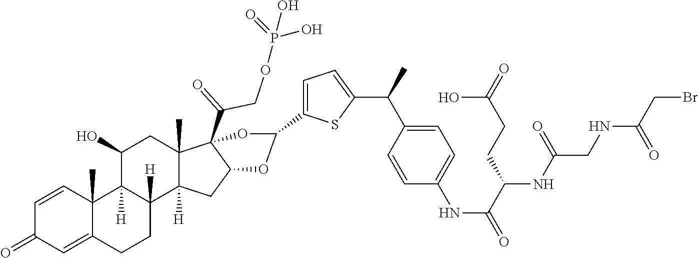 |
| II- D- 427 | 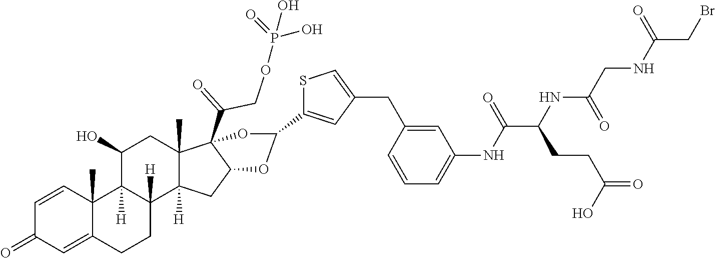 |
| II- D- 439 | 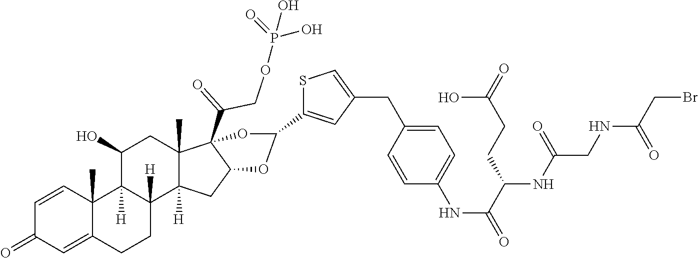 |
| II- D- 451 | 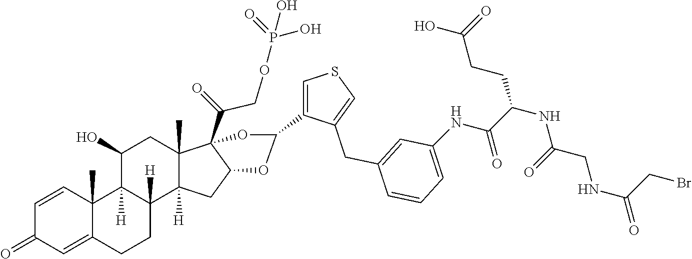 |
| II- D- 452 | 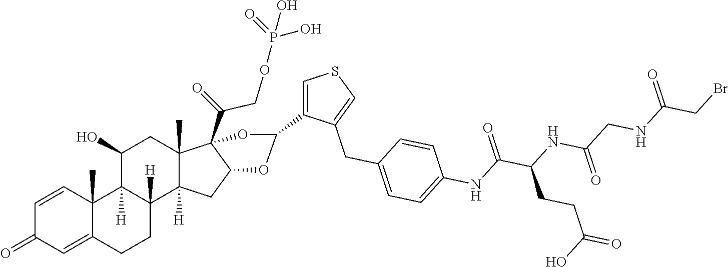 |
| II- D- 453 | 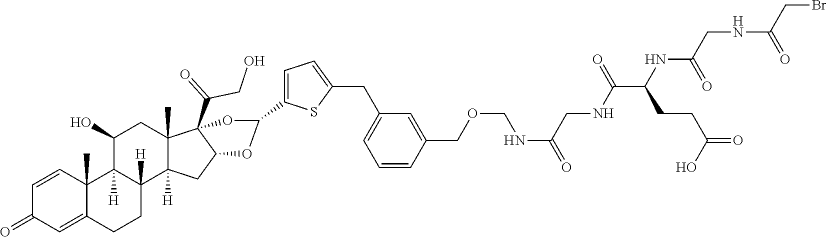 |
| II- D- 454 | 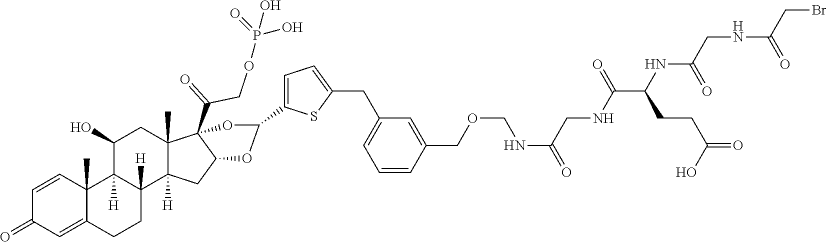 |
| II- D- 455 | 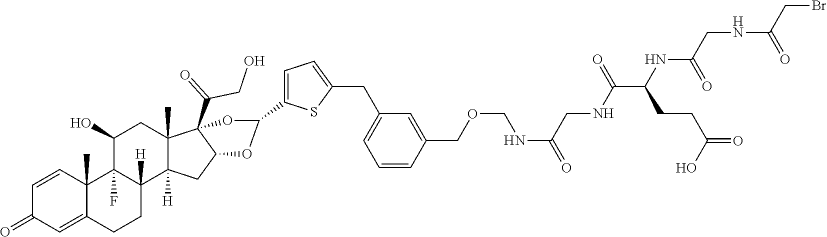 |
| II- D- 456 | 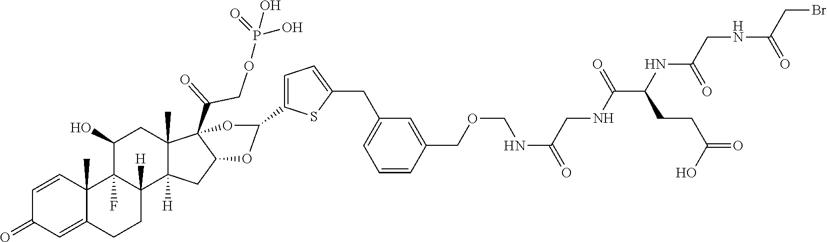 |
| II- D- 457 | 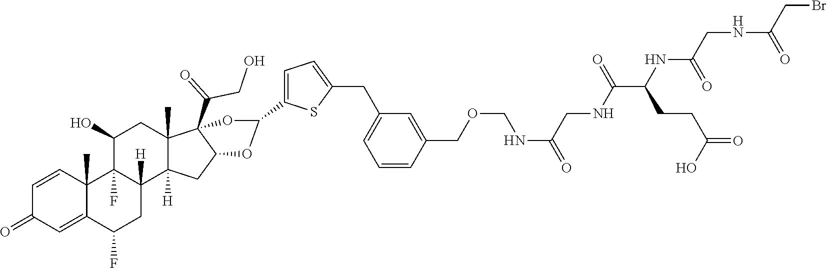 |
| II- D- 458 | 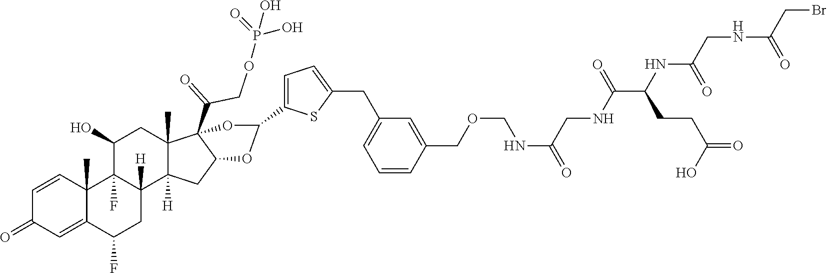 |
| II- D- 459 | 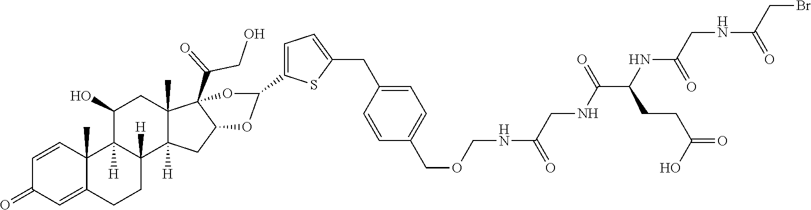 |
| II- D- 460 | 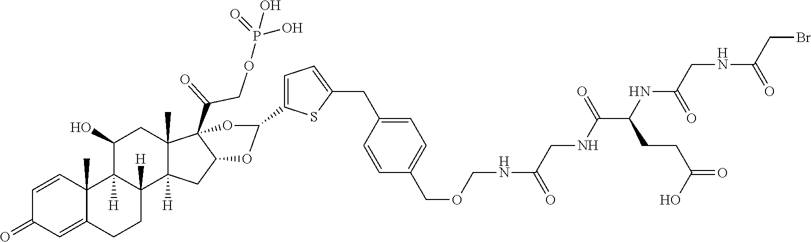 |
| II- D- 461 | 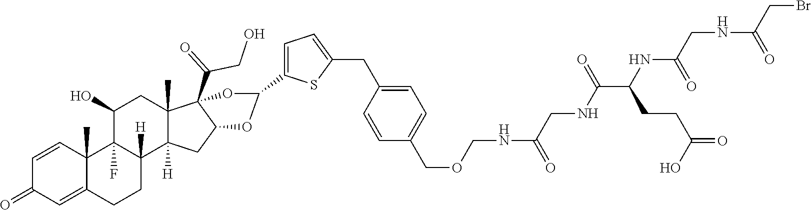 |
| II- D- 462 | 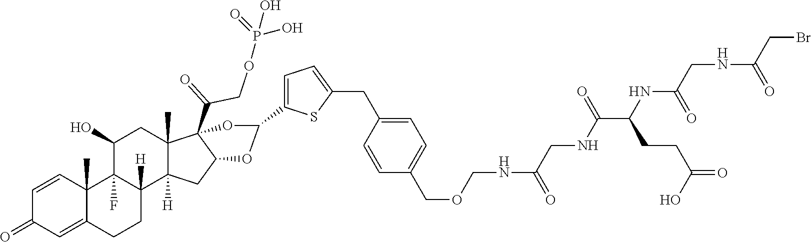 |
| II- D- 463 | 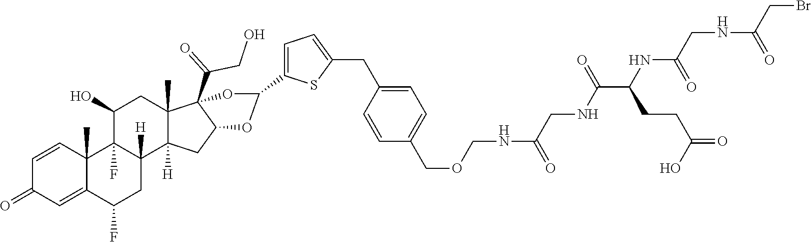 |
| II- D- 464 | 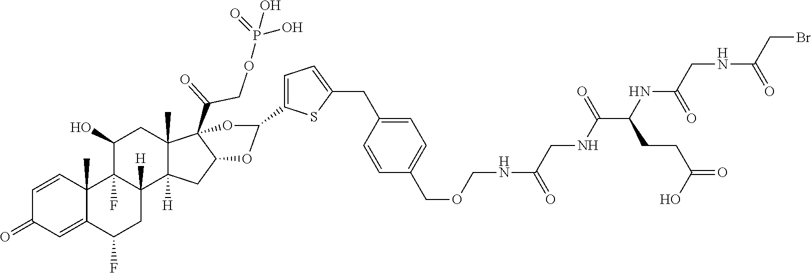 |
| II- D- 465 | 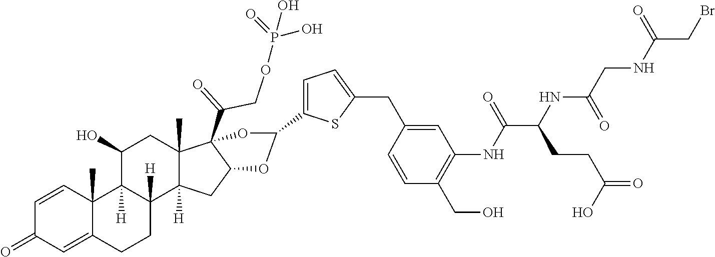 |
| II- D- 468 | 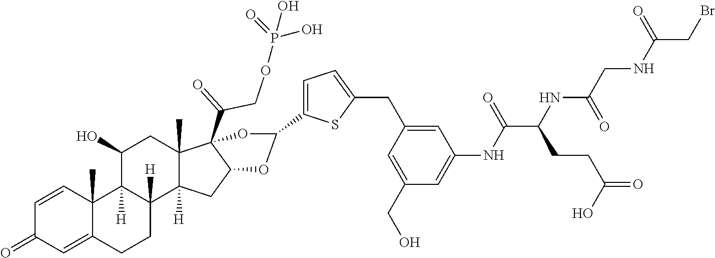 |
| II- D- 471 | 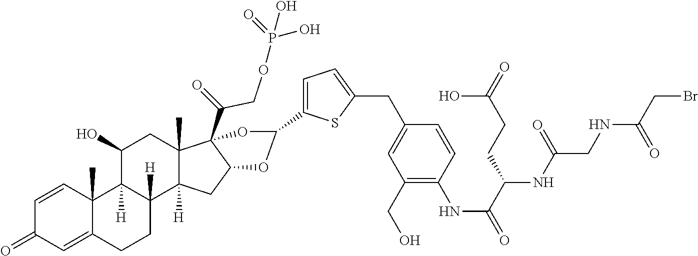 |
| II- D- 474 | 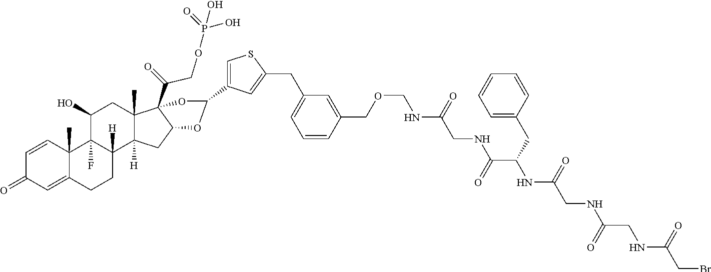 |
| II- D- 475 | 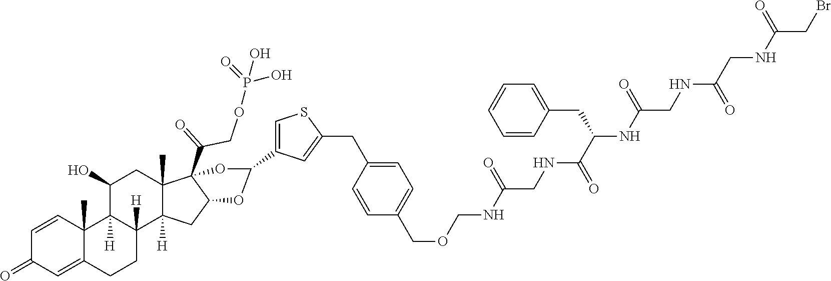 |
| II- D- 476 | 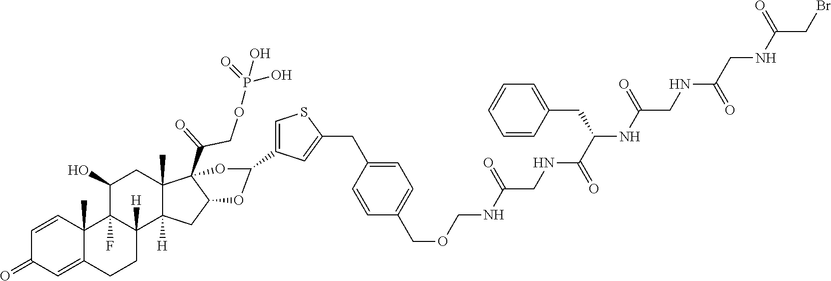 |
| II- D- 477 | 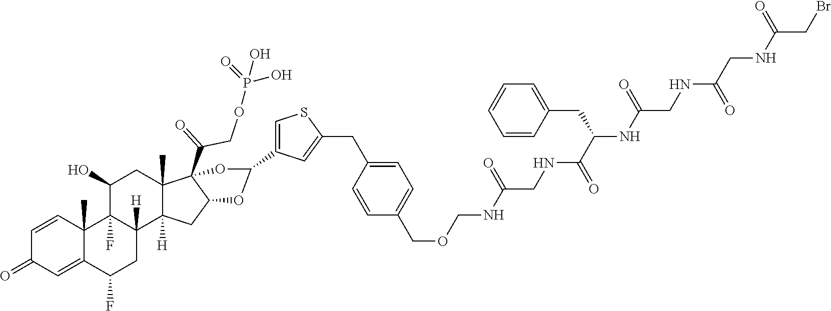 |
| II- D- 478 | 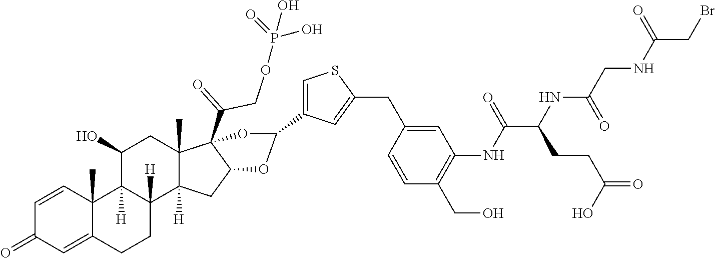 |
| II- D- 481 | 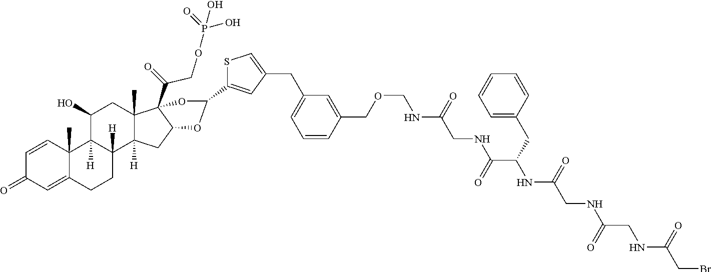 |
| II- D- 482 | 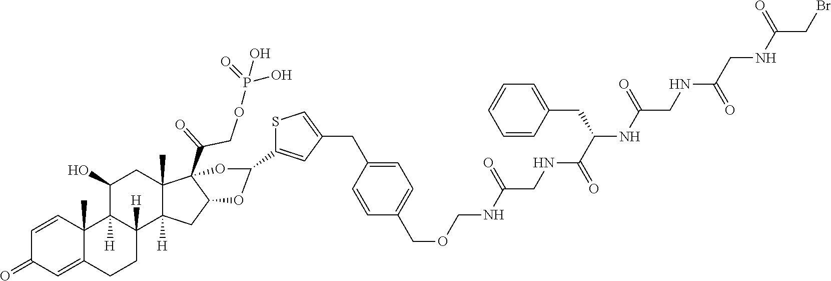 |
| II- D- 483 | 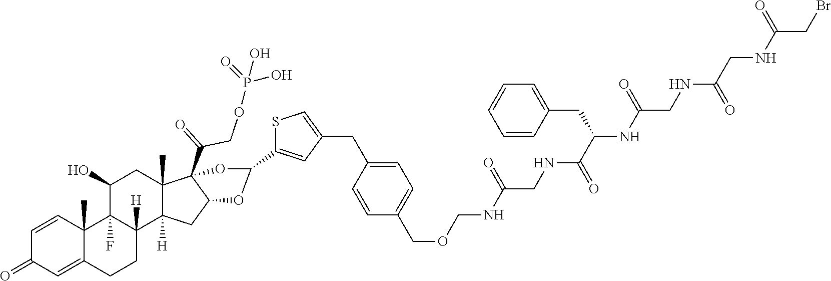 |
| II- D- 484 | 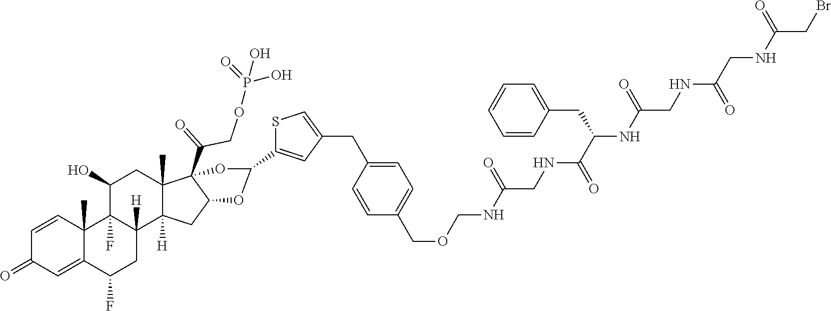 |
| II- D- 485 | 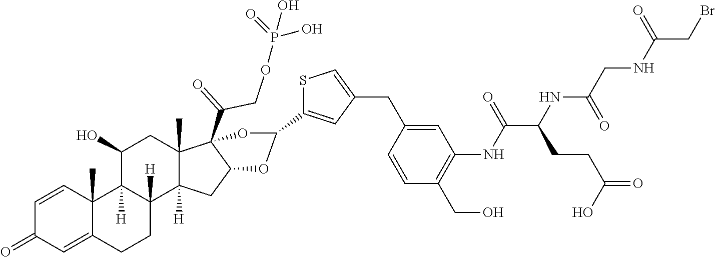 |
| II- D- 488 | 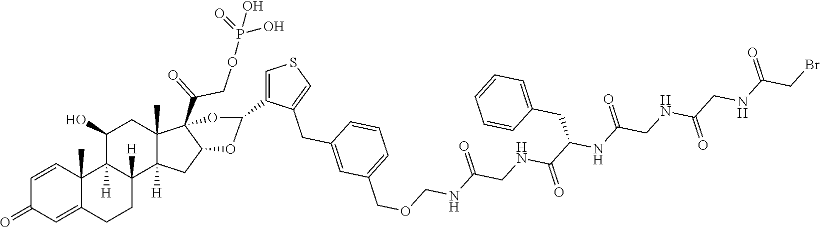 |
| II- D- 489 | 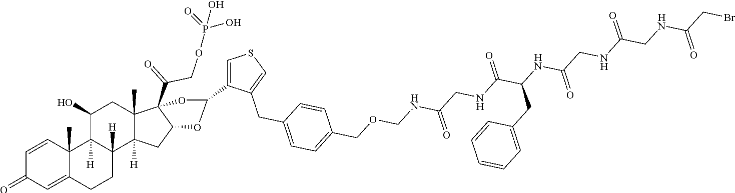 |
| II- D- 505 | 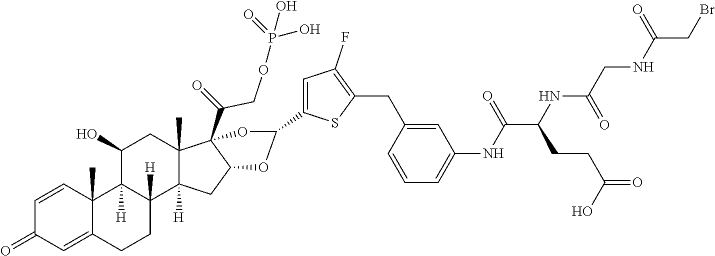 |
| II- D- 508 | 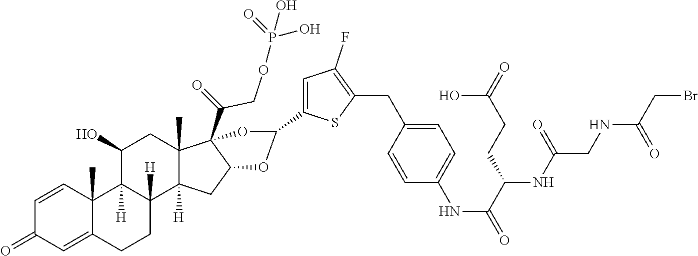 |
| II- D- 511 | 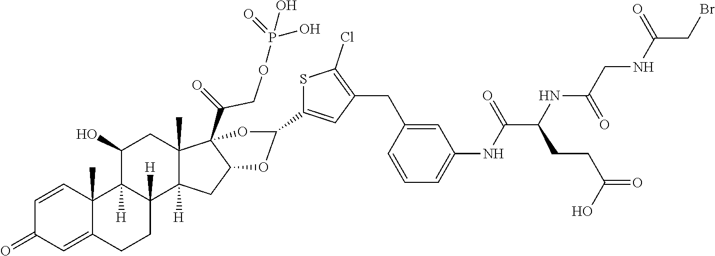 |
| II- D- 514 | 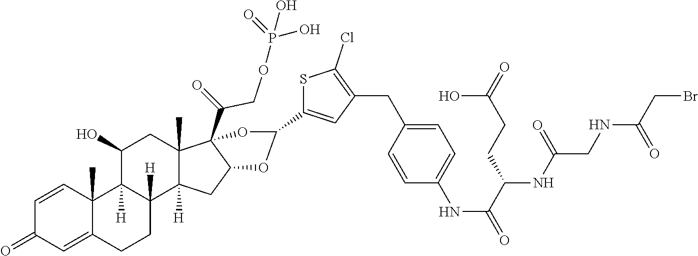 |
| II- D- 517 | 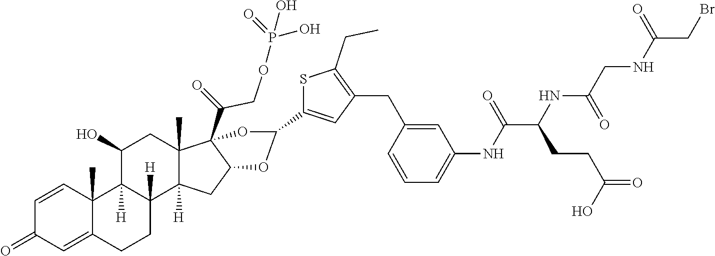 |
| II- D- 520 | 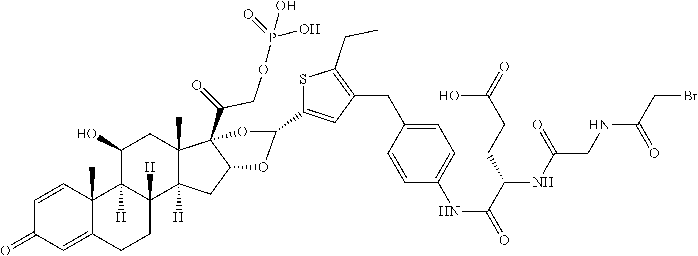 |
| II- D- 523 | 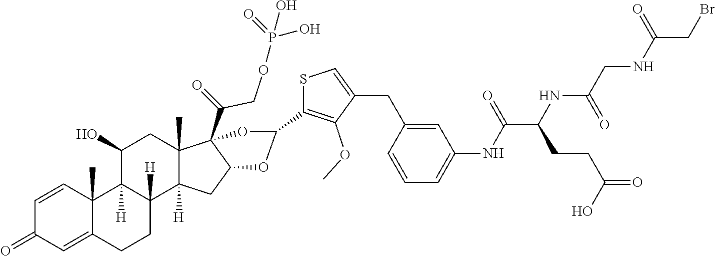 |
| II- D- 526 | 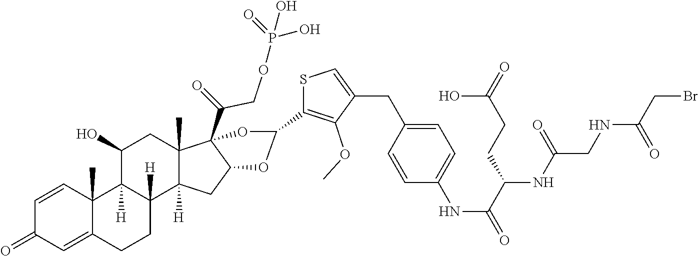 |
| II- D- 529 | 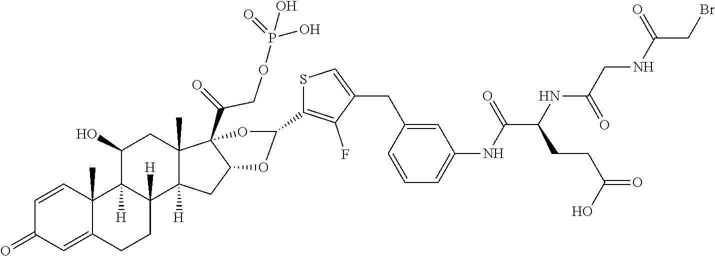 |
| II- D- 532 | 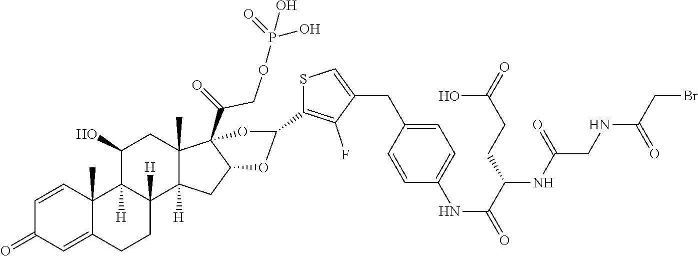 |
| II- D- 535 | 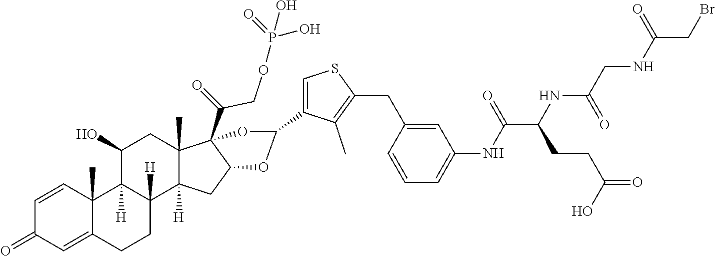 |
| II- D- 538 | 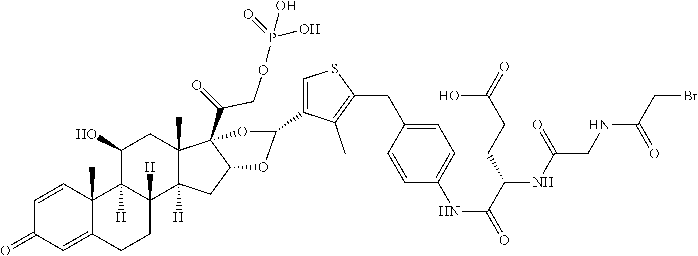 |
| II- D- 541 | 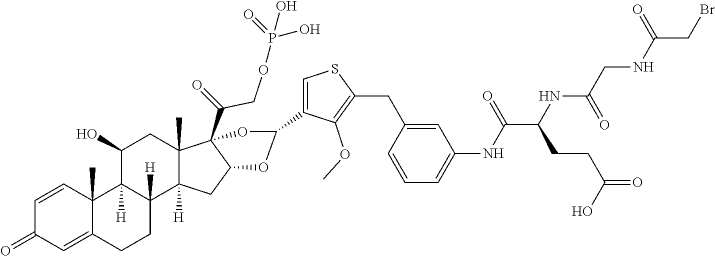 |
| II- D- 544 | 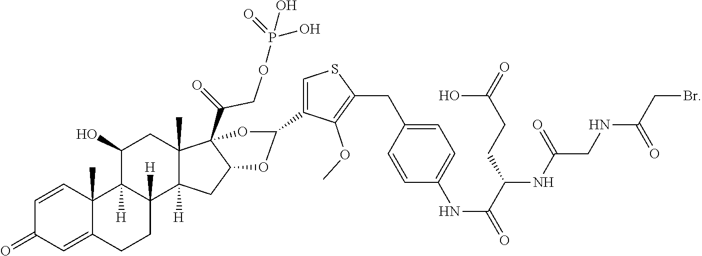 |

79. A compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

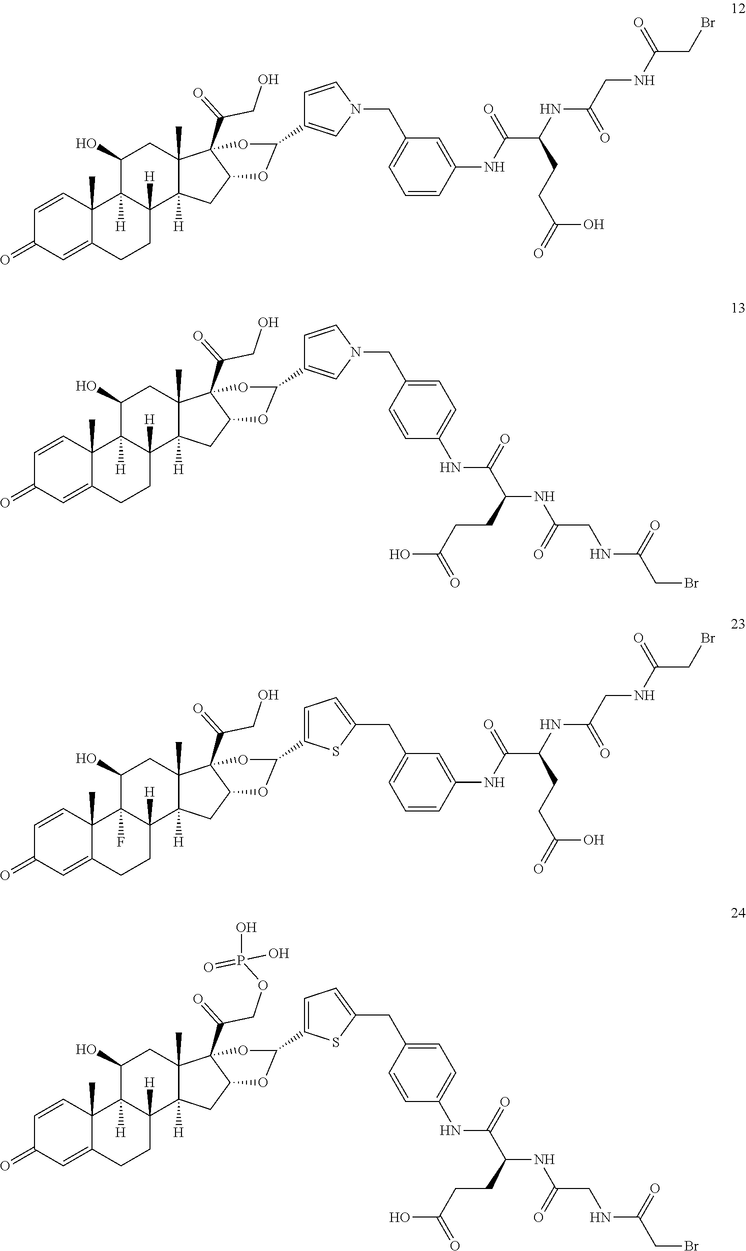

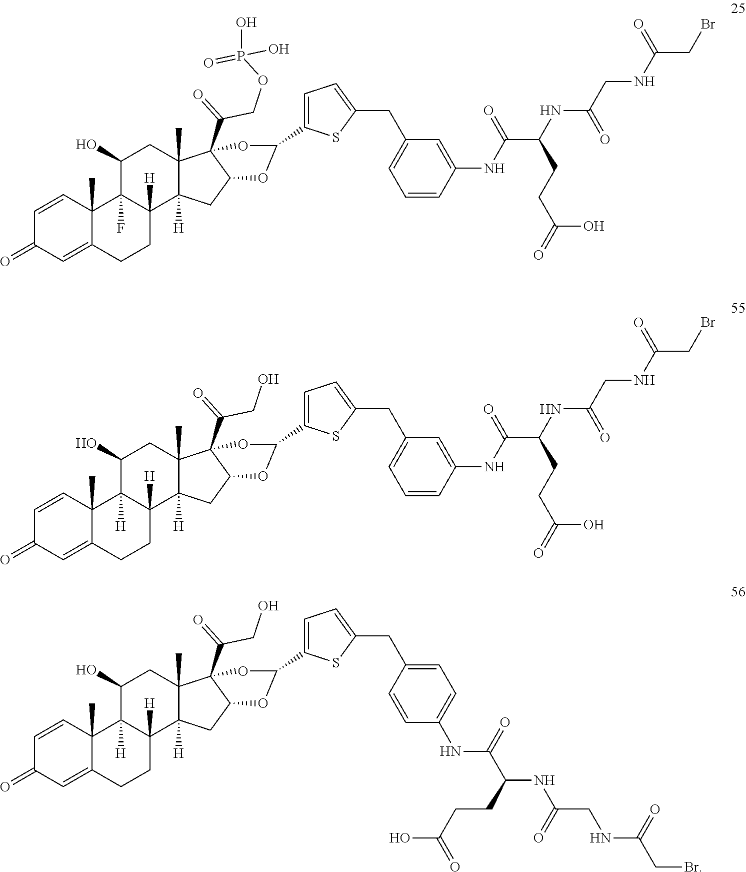

80. A compound or a tautomer, a mesomer, a racemate, an enantiomer or a diastereoisomer thereof, or a mixture thereof, or a pharmaceutically acceptable salt thereof, wherein the compound comprises a structure selected from the group consisting of:

| No. | Structure |
|---|---|
| II-C- 1 | 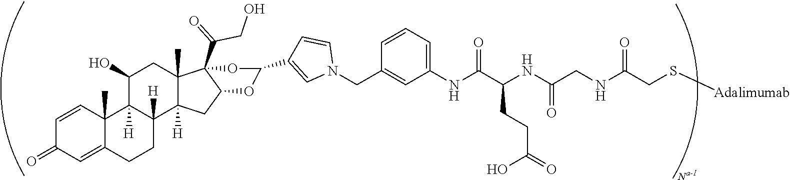 |
| II-C- 2 | 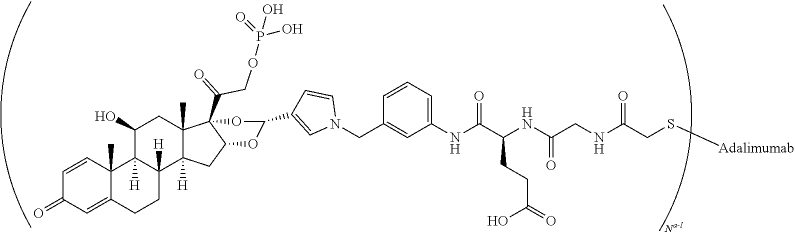 |
| II-C- 7 | 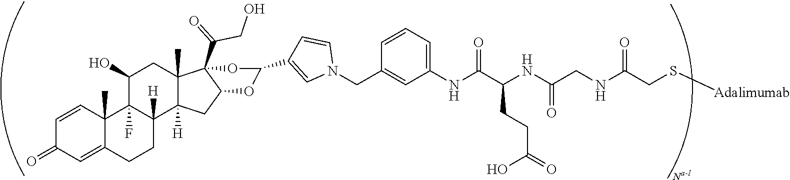 |
| II-C- 8 | 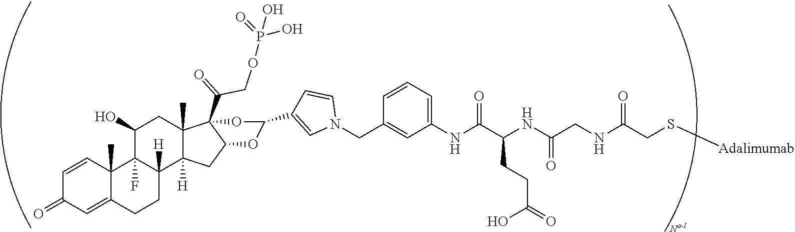 |
| II-C- 12 | 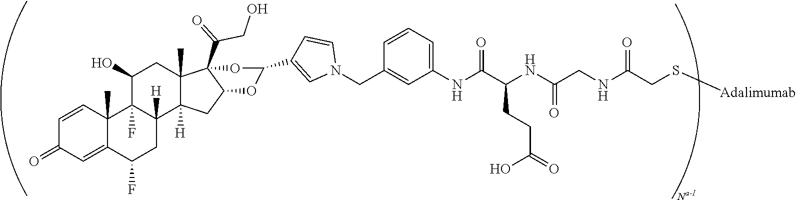 |
| II-C- 13 | 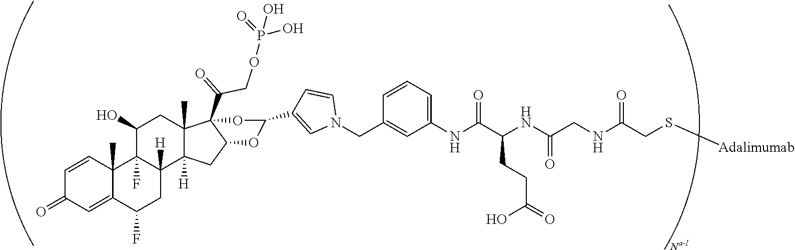 |
| II-C- 17 | 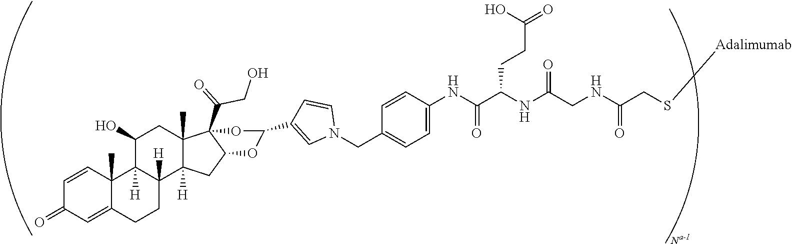 |
| II-C- 18 | 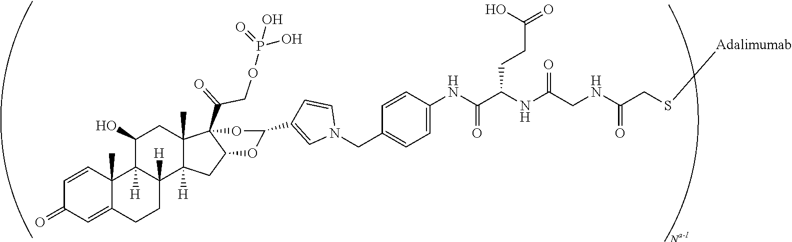 |
| II-C- 23 | 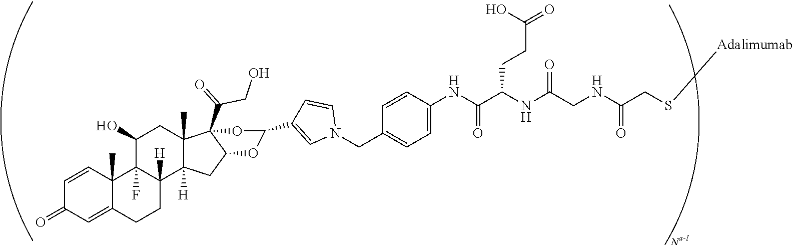 |
| II-C- 24 | 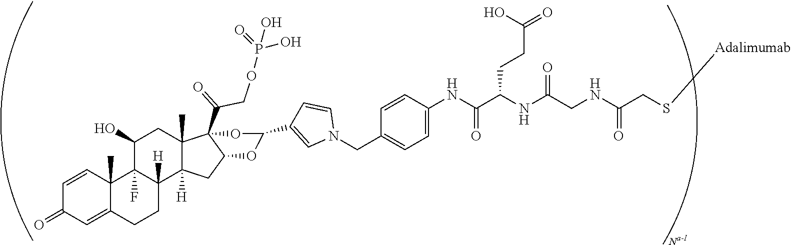 |
| II-C- 29 | 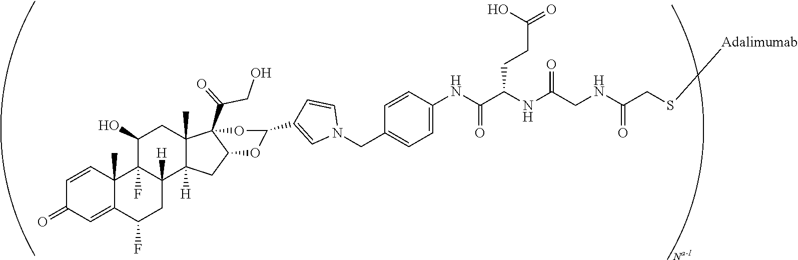 |
| II-C- 30 | 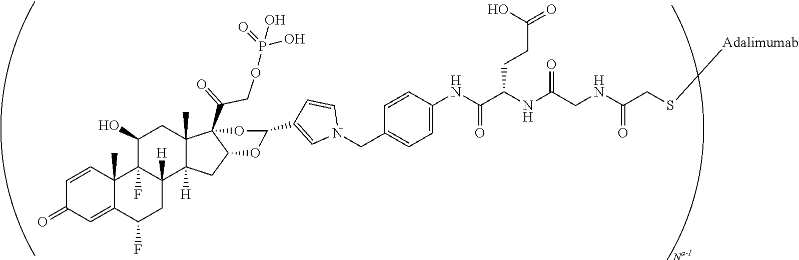 |
| II-C- 34 | 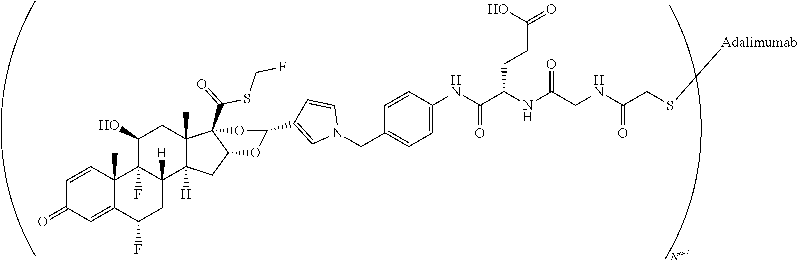 |
| II-C- 35 | 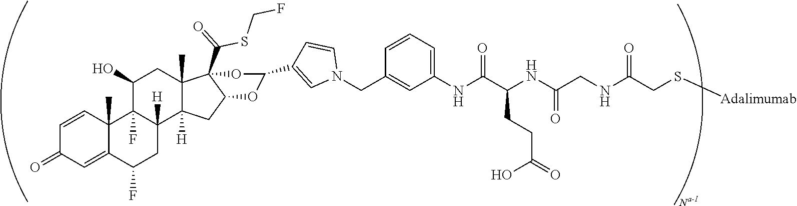 |
| II-C- 36 | 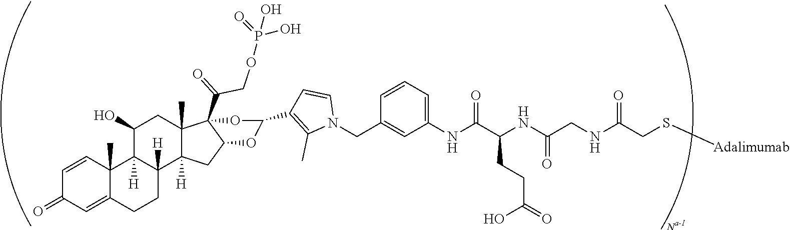 |
| II-C- 39 | 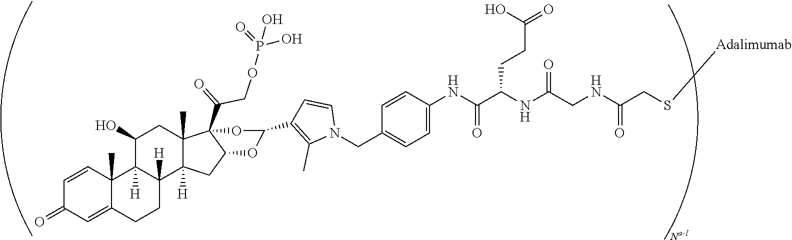 |
| II-C- 42 | 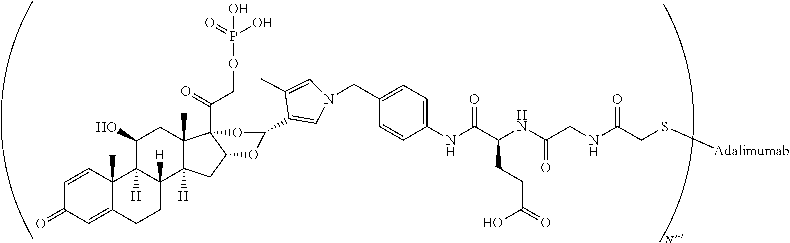 |
| II-C- 45 | 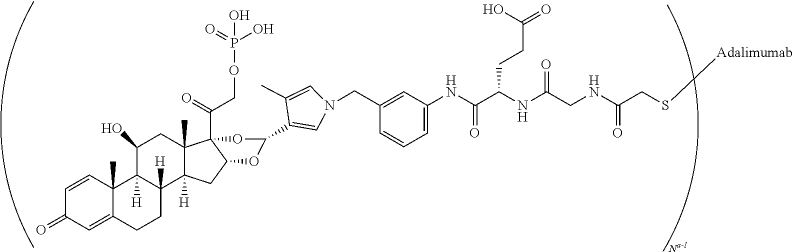 |
| II-C- 48 | 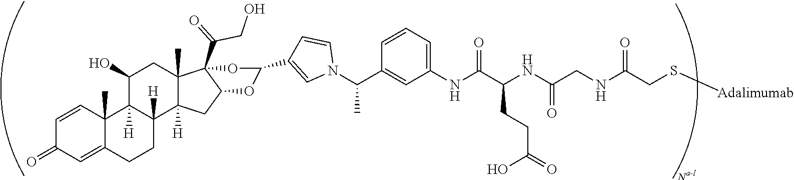 |
| II-C- 49 | 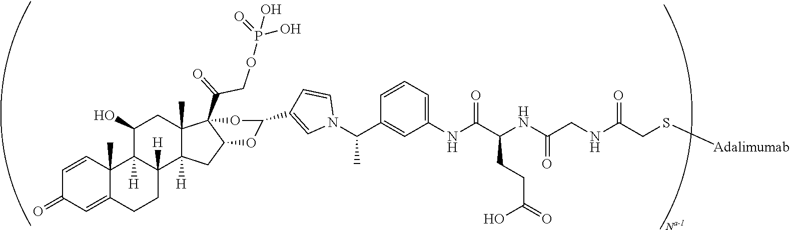 |
| II-C- 53 | 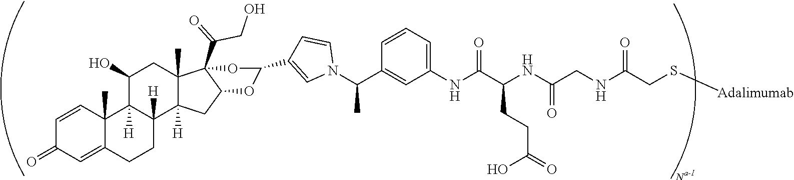 |
| II-C- 54 | 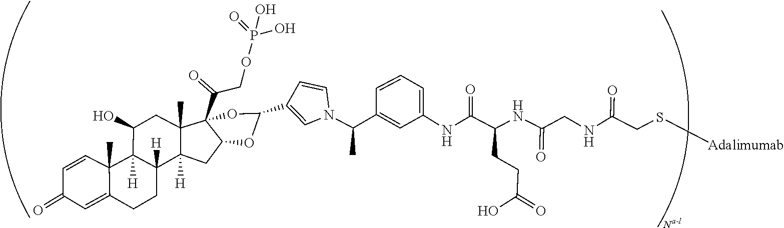 |
| II-C- 78 | 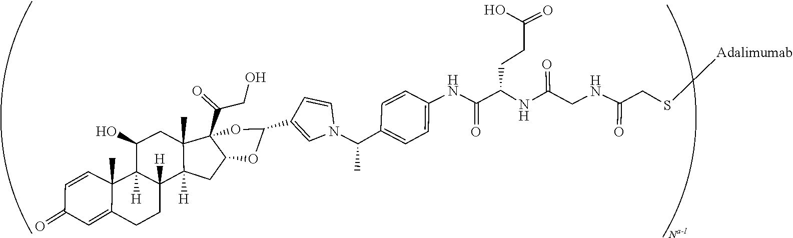 |
| II-C- 79 | 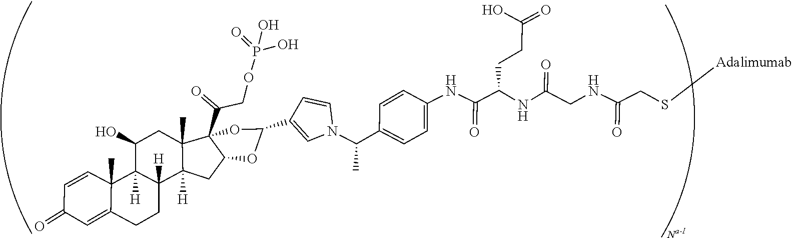 |
| II-C- 92 | 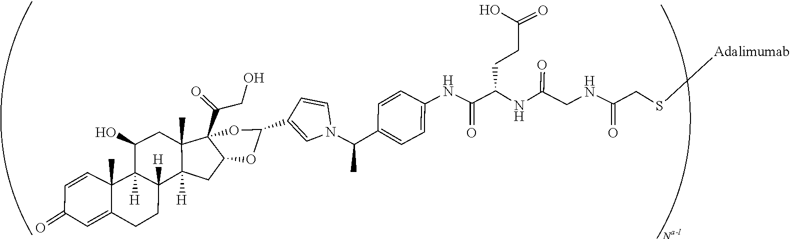 |
| II-C- 93 | 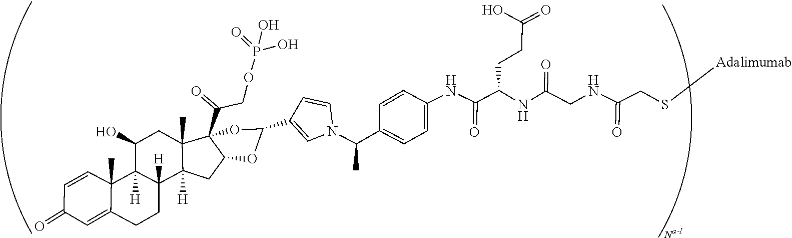 |
| II-C- 107 | 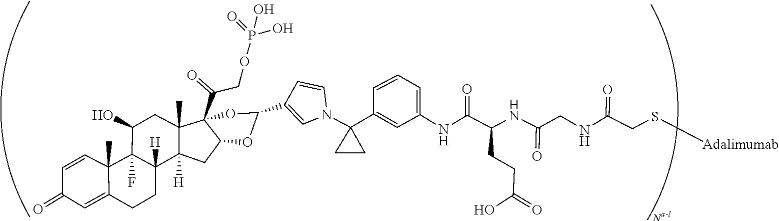 |
| II-C- 108 | 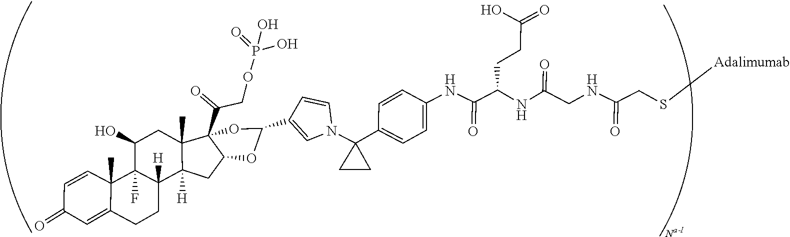 |
| II-C- 111 | 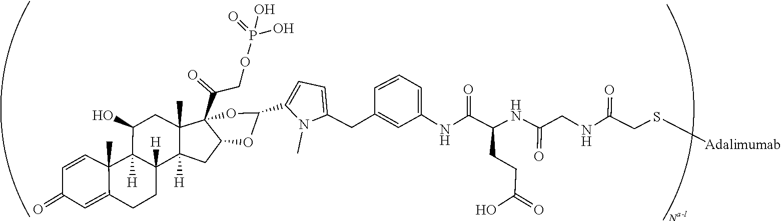 |
| II-C- 114 | 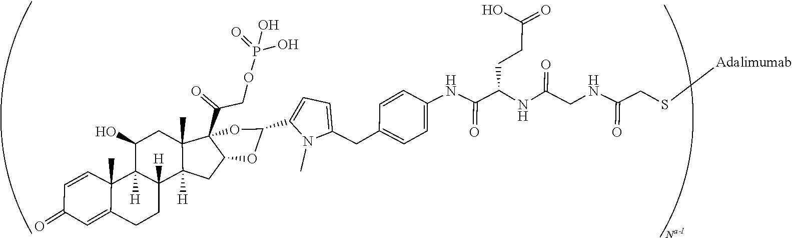 |
| II-C- 117 | 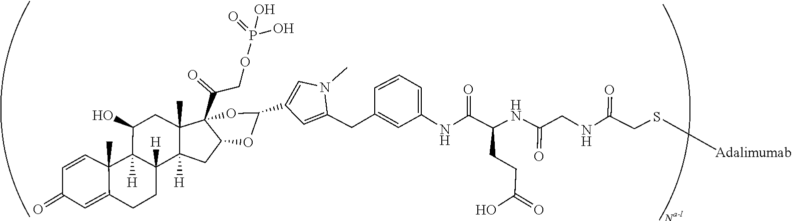 |
| II-C- 120 | 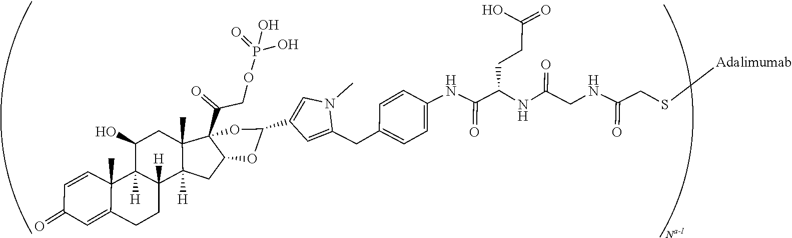 |
| II-C- 123 | 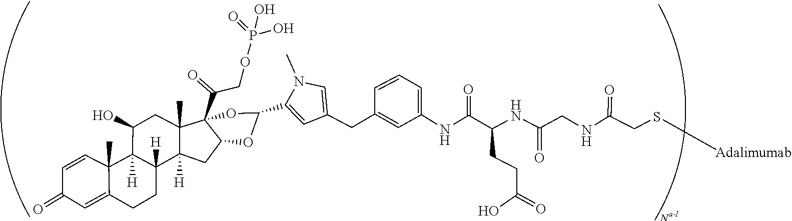 |
| II-C- 126 | 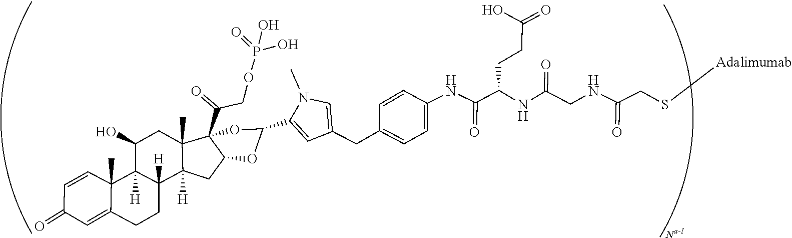 |
| II-C- 165 | 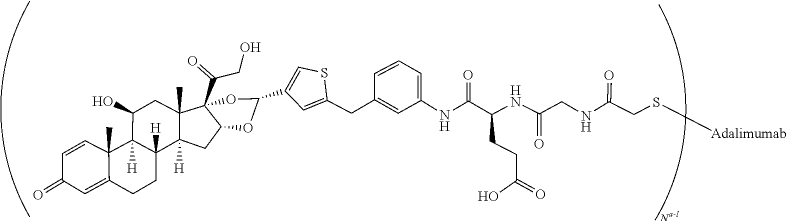 |
| II-C- 166 | 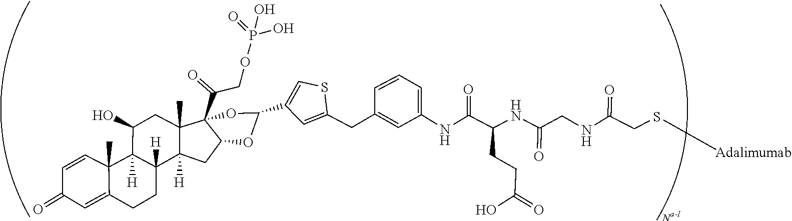 |
| II-C- 170 | 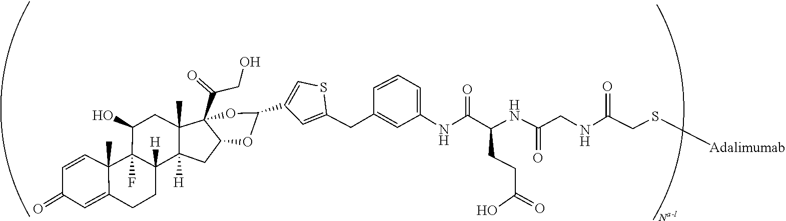 |
| II-C- 171 | 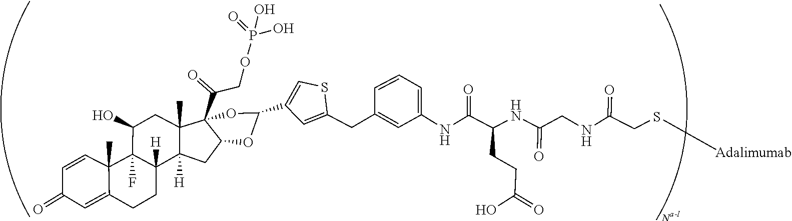 |
| II-C- 175 | 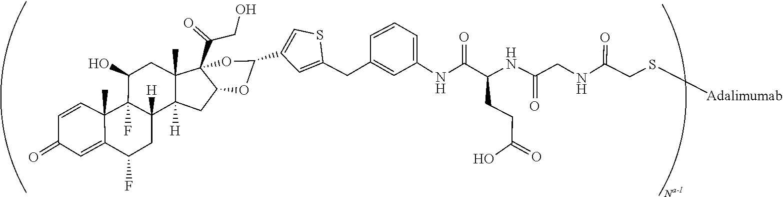 |
| II-C- 176 | 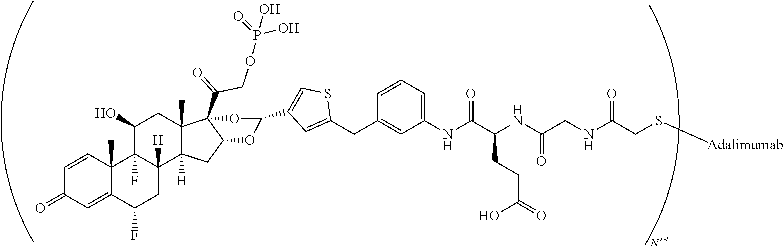 |
| II-C- 180 | 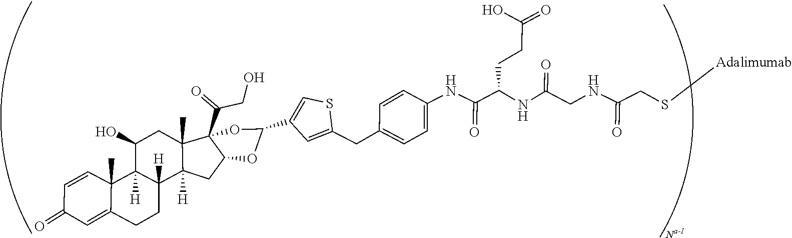 |
| II-C- 181 | 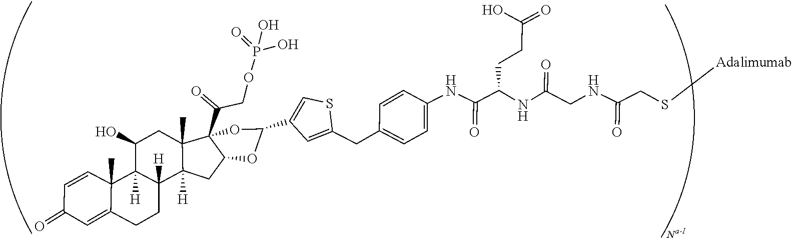 |
| II-C- 185 | 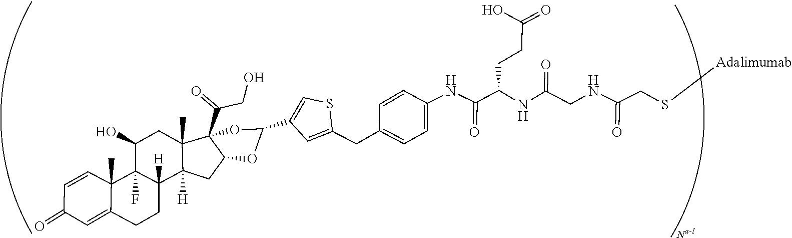 |
| II-C- 186 | 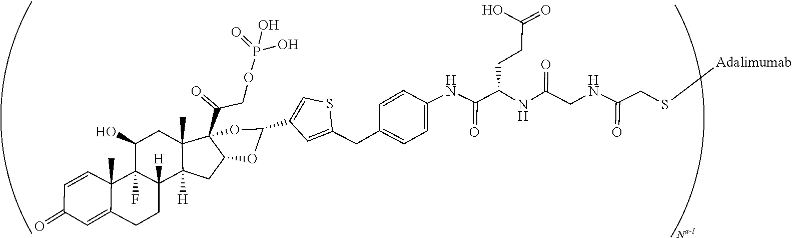 |
| II-C- 190 | 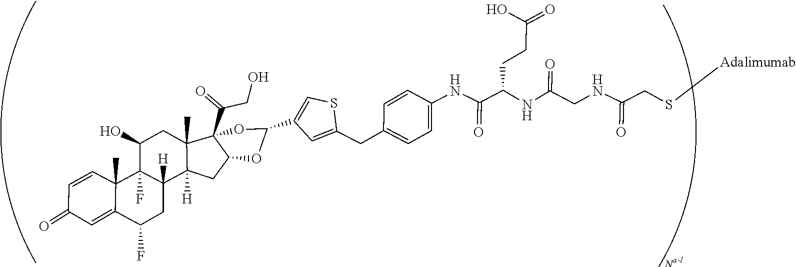 |
| II-C- 191 | 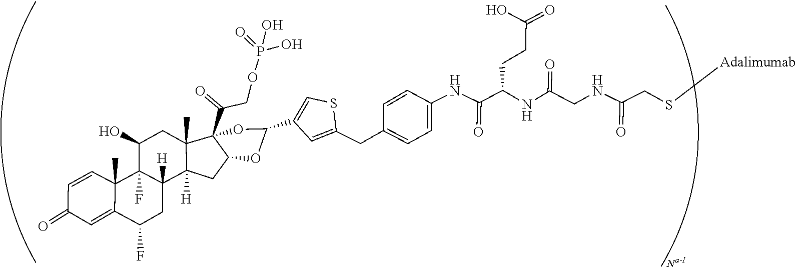 |
| II-C- 195 | 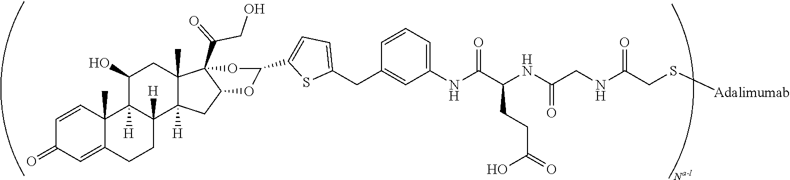 |
| II-C- 196 | 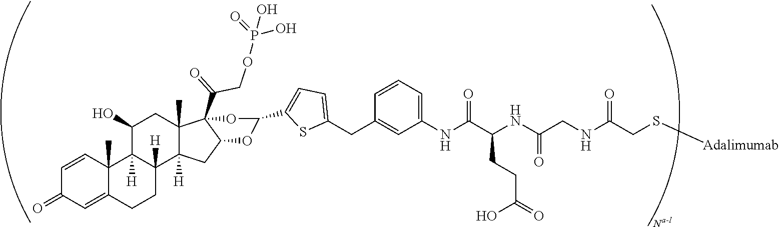 |
| II-C- 200 | 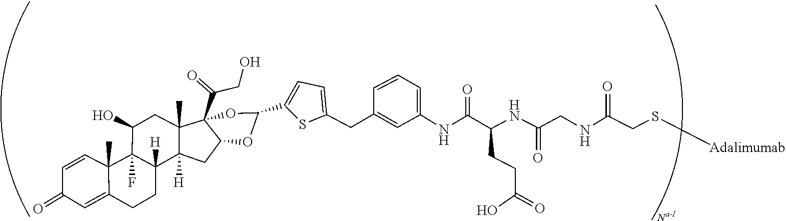 |
| II-C- 201 | 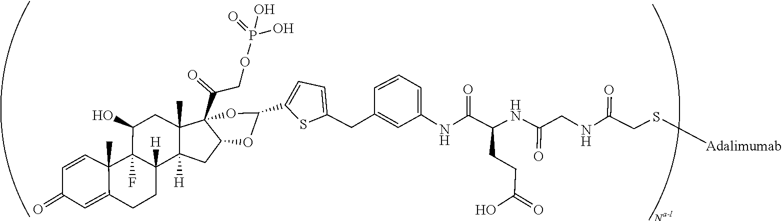 |
| II-C- 205 | 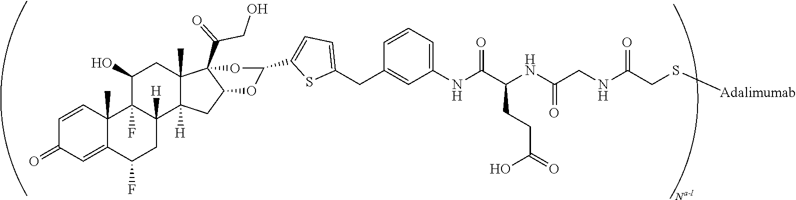 |
| II-C- 206 | 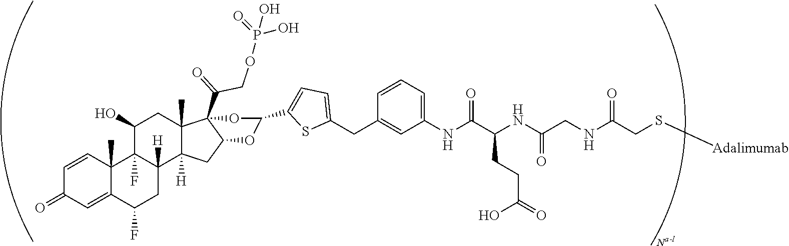 |
| II-C- 210 | 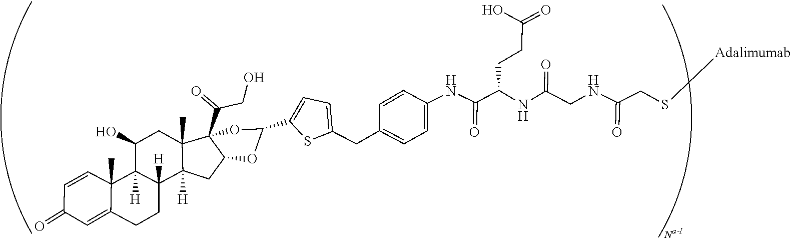 |
| II-C- 211 | 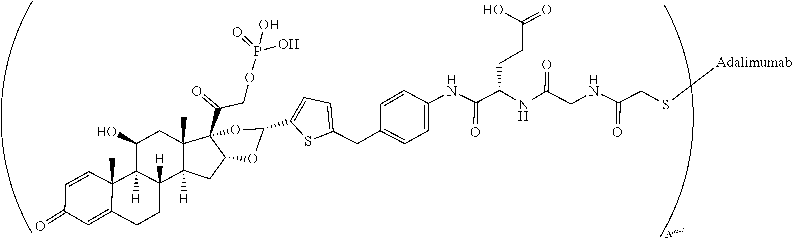 |
| II-C- 215 | 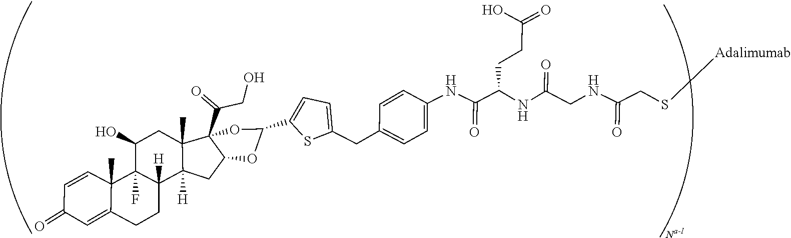 |
| II-C- 216 | 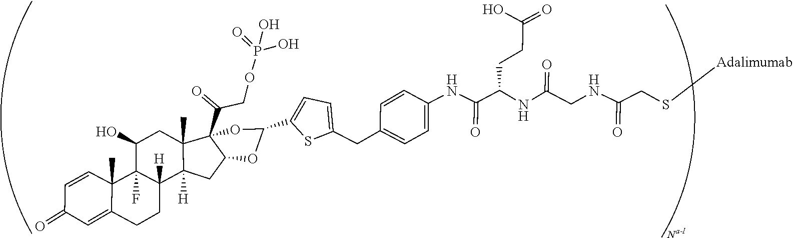 |
| II-C- 220 | 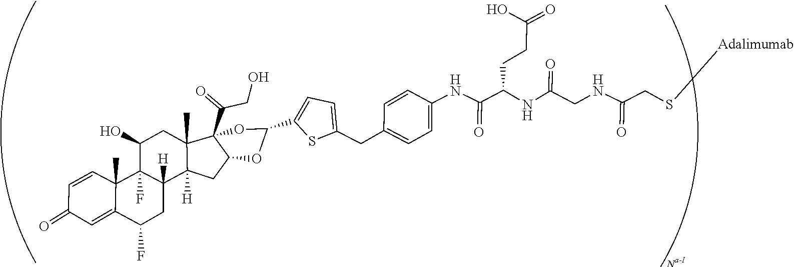 |
| II-C- 221 | 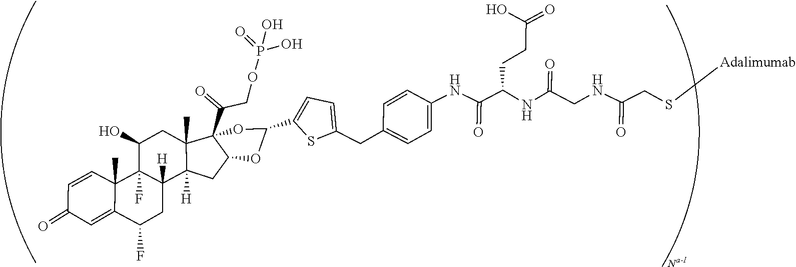 |
| II-C- 233 | 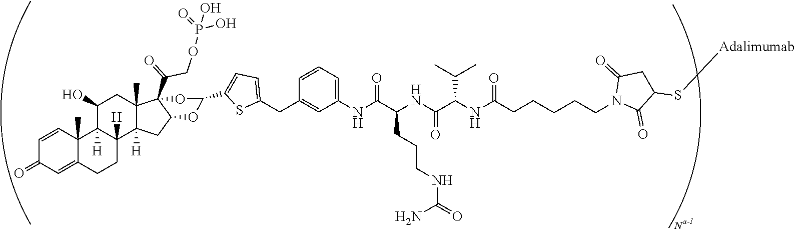 |
| II-C- 234 | 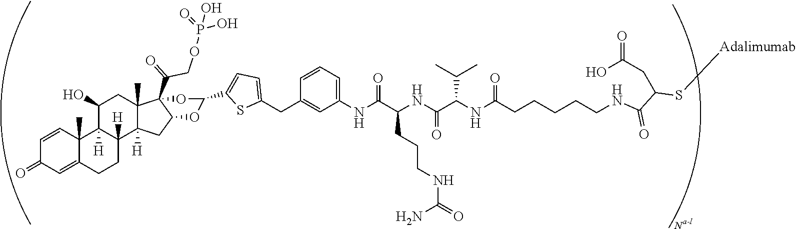 |
| II-C- 235 | 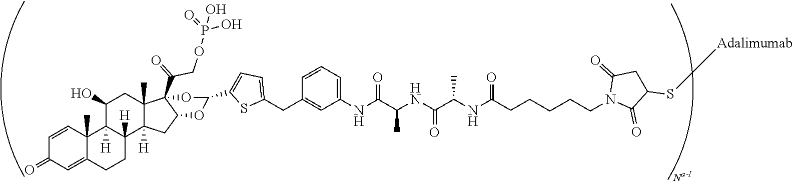 |
| II-C- 236 | 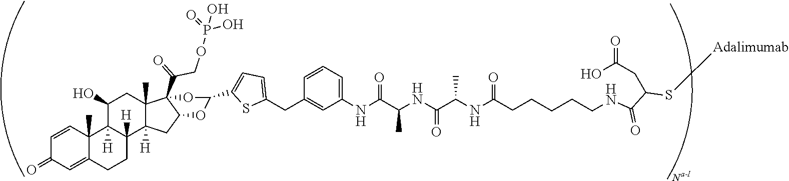 |
| II-C- 237 | 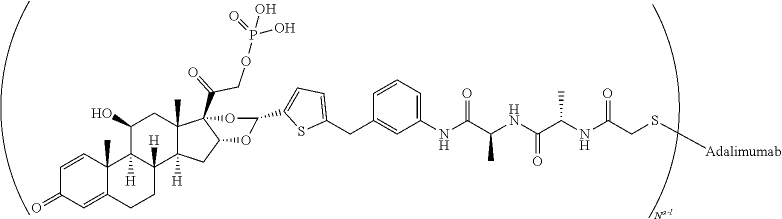 |
| II-C- 238 | 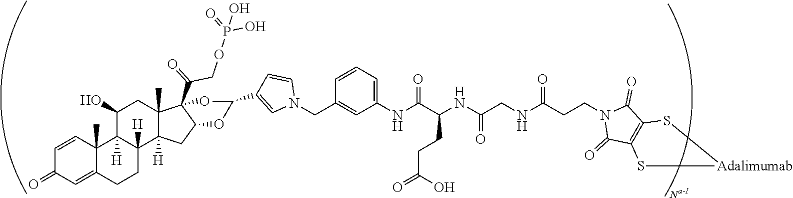 |
| II-C- 239 | 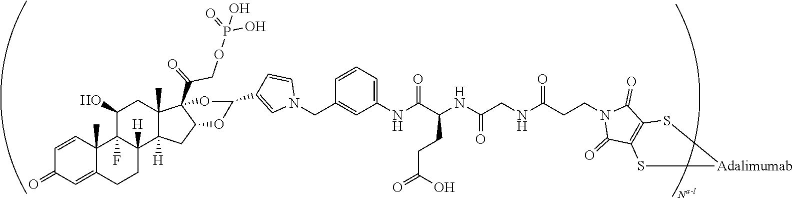 |
| II-C- 240 | 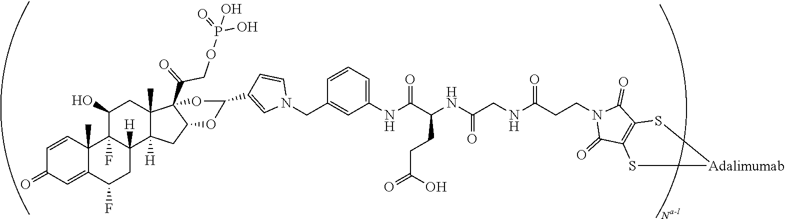 |
| II-C- 241 | 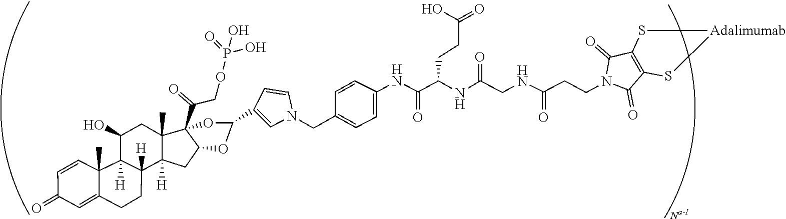 |
| II-C- 242 | 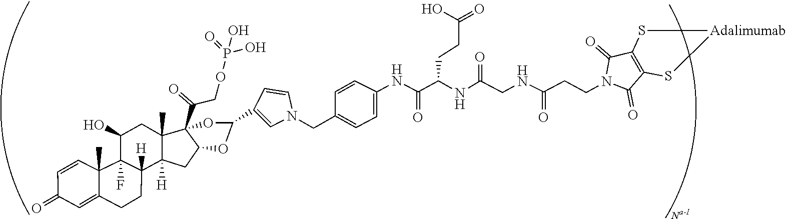 |
| II-C- 243 | 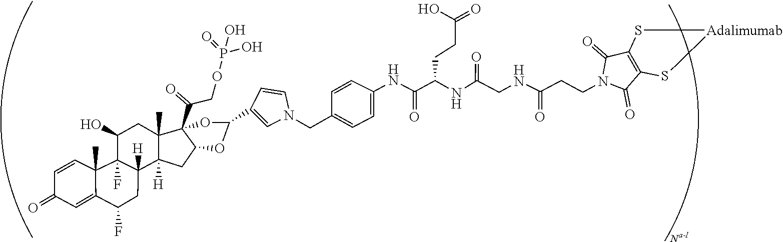 |
| II-C- 244 | 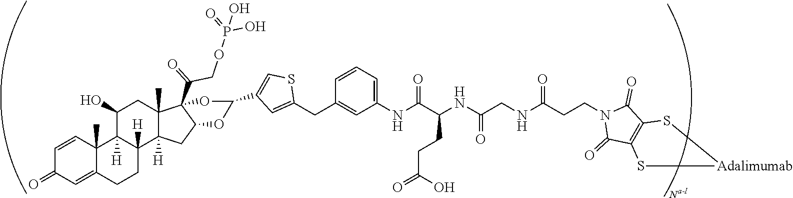 |
| II-C- 245 | 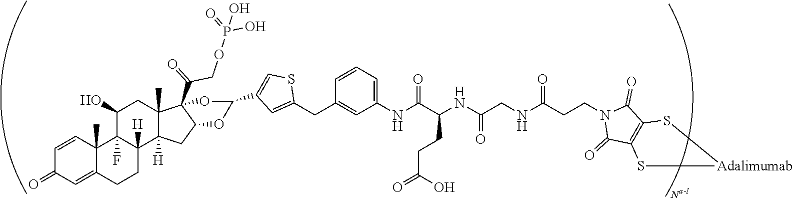 |
| II-C- 246 | 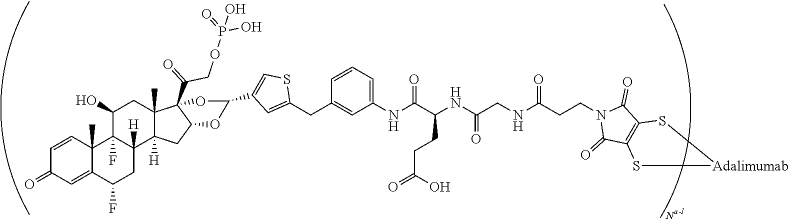 |
| II-C- 247 | 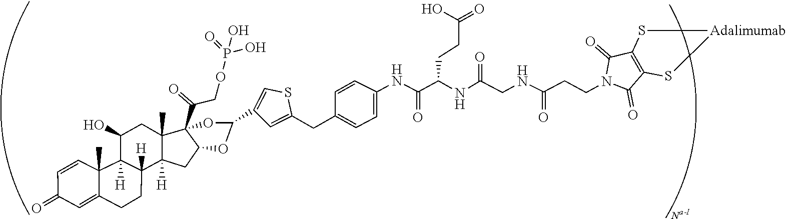 |
| II-C- 248 | 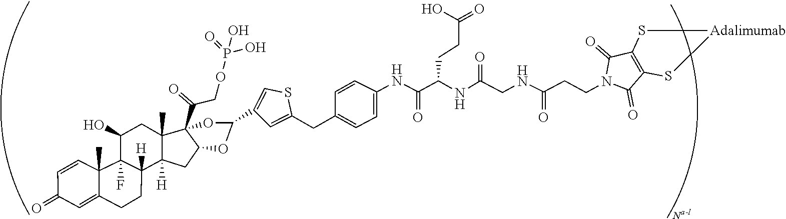 |
| II-C- 249 | 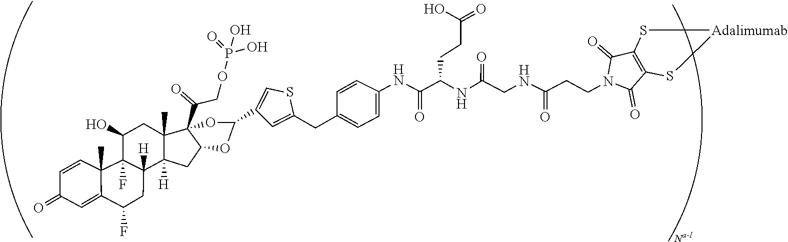 |
| II-C- 250 | 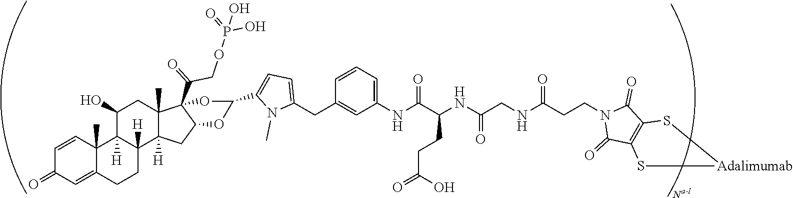 |
| II-C- 253 | 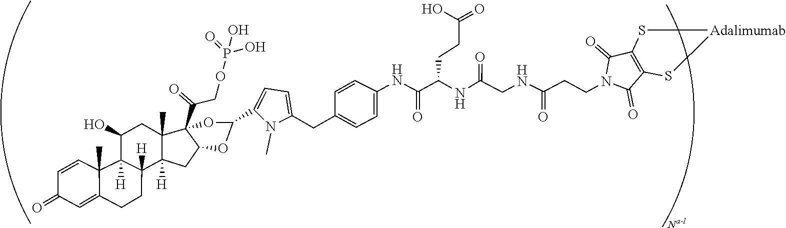 |
| II-C- 256 | 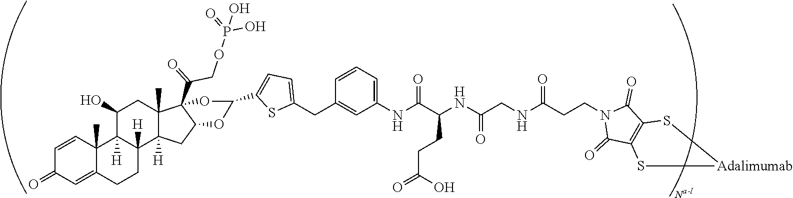 |
| II-C- 257 | 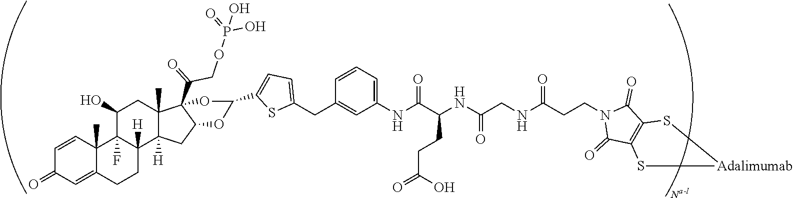 |
| II-C- 258 | 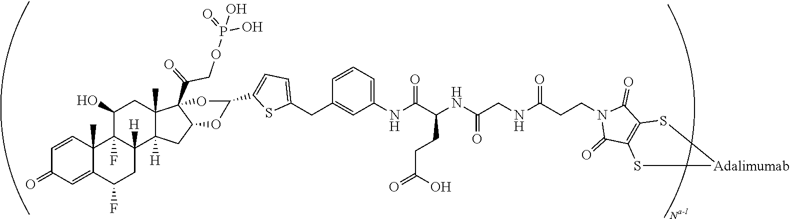 |
| II-C- 259 | 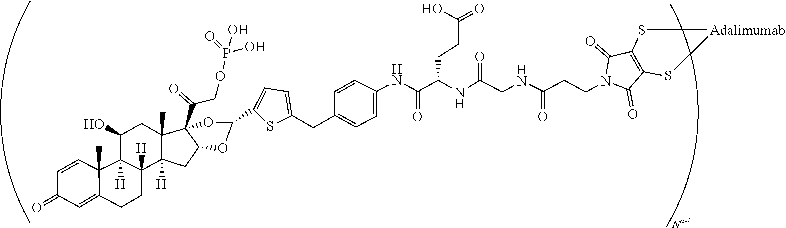 |
| II-C- 260 | 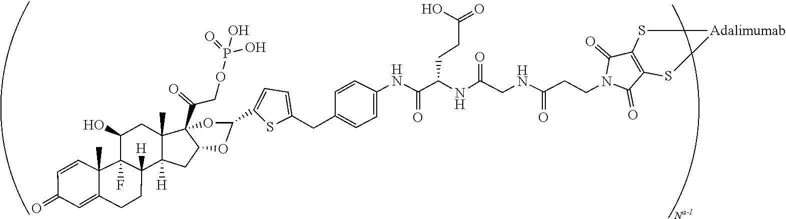 |
| II-C- 261 | 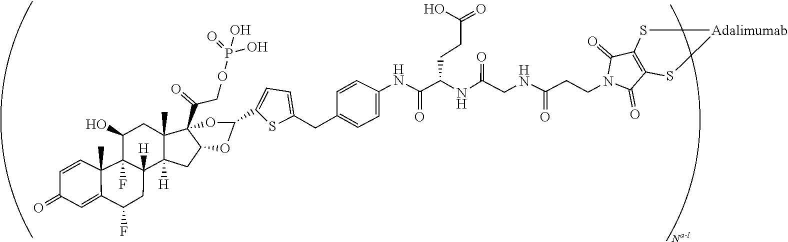 |
| II-C- 262 | 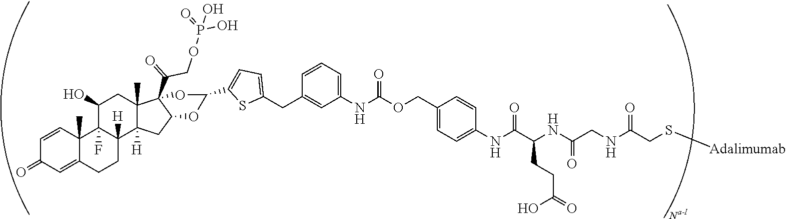 |
| II-C- 263 | 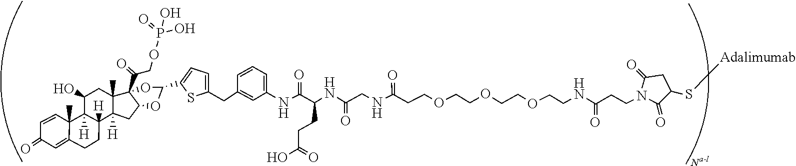 |
| II-C- 264 | 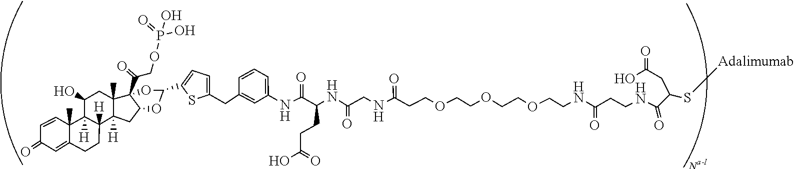 |
| II-C- 265 | 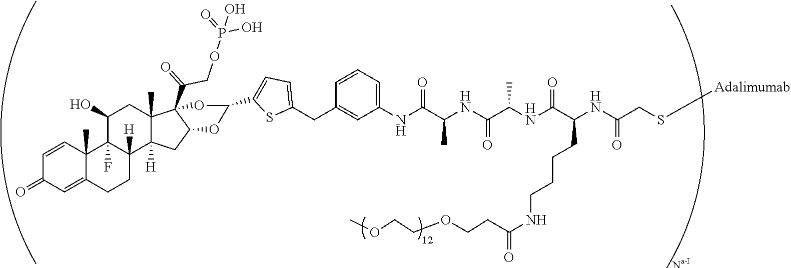 |
| II-C- 266 | 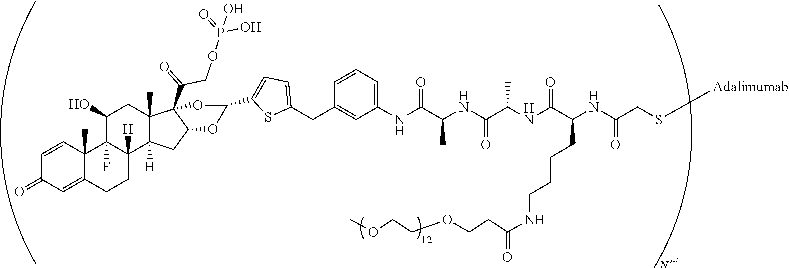 |
| II-C- 267 | 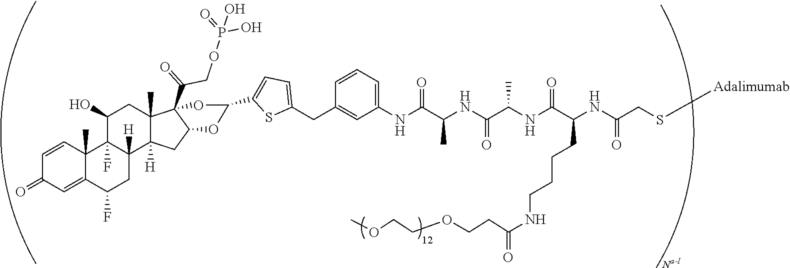 |
| II-C- 268 | 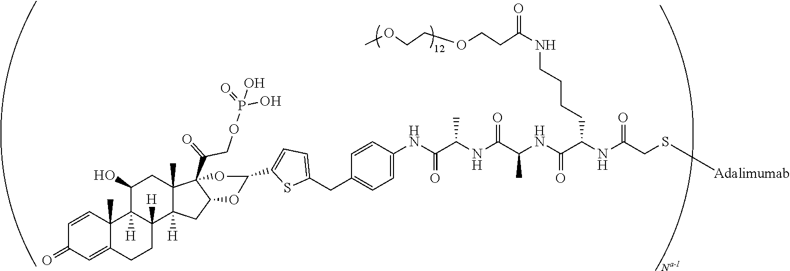 |
| II-C- 269 | 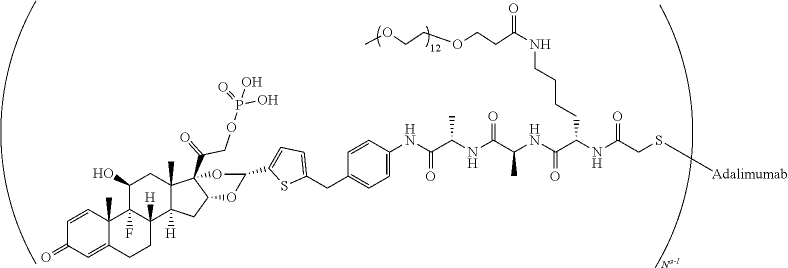 |
| II-C- 270 | 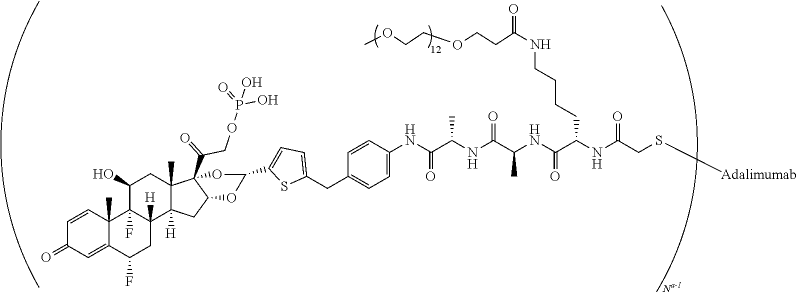 |
| II-C- 271 | 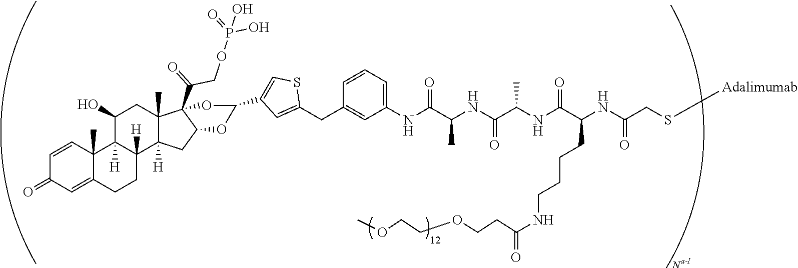 |
| II-C- 272 | 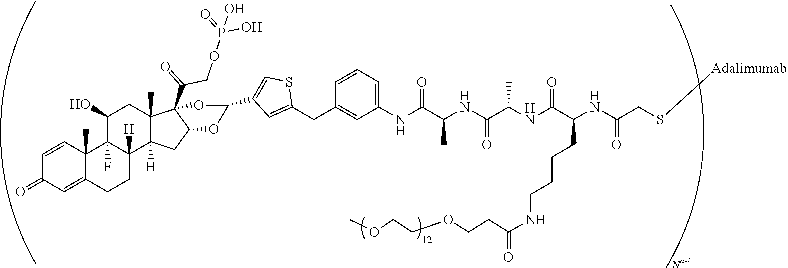 |
| II-C- 273 | 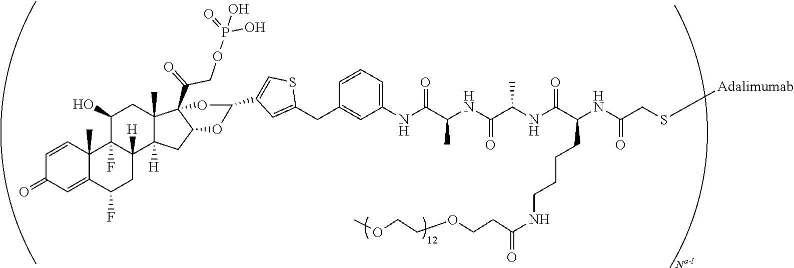 |
| II-C- 274 | 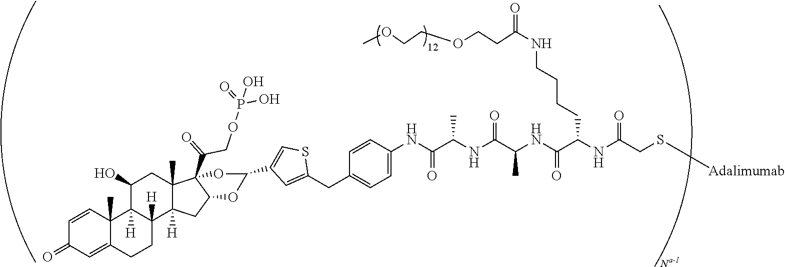 |
| II-C- 275 | 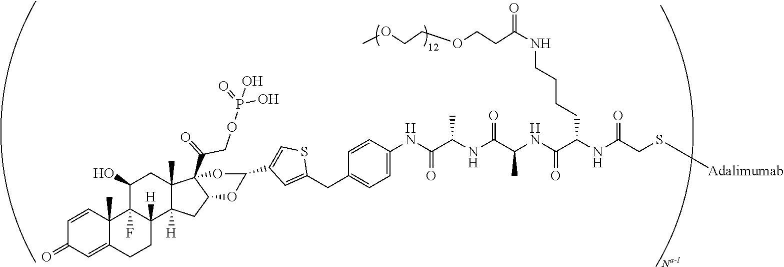 |
| II-C- 276 | 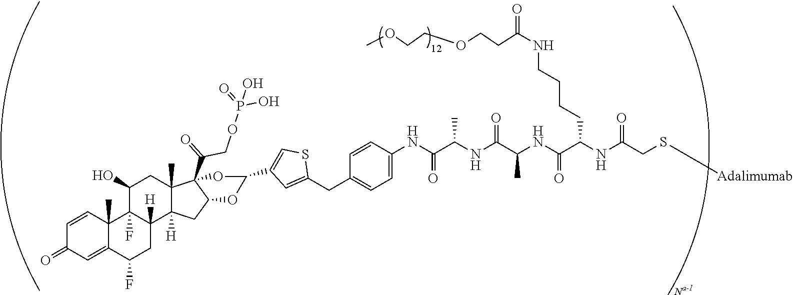 |
| II-C- 277 | 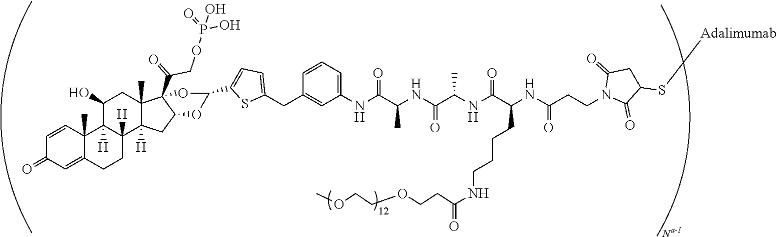 |
| II-C- 278 | 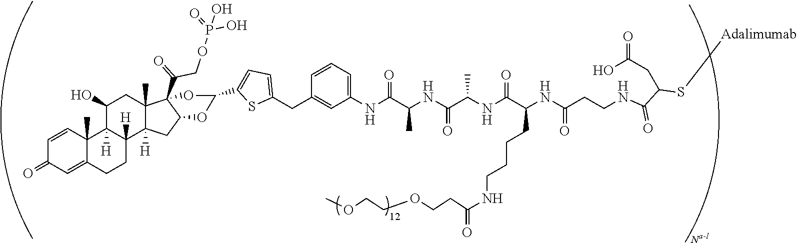 |
| II-C- 279 | 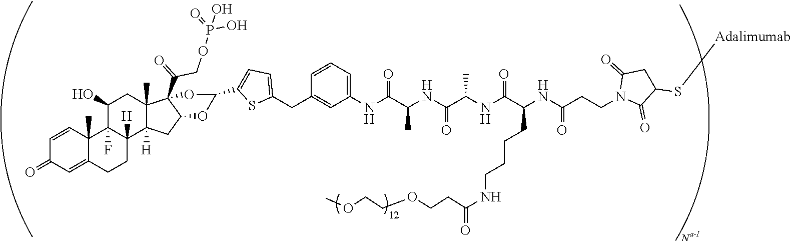 |
| II-C- 280 | 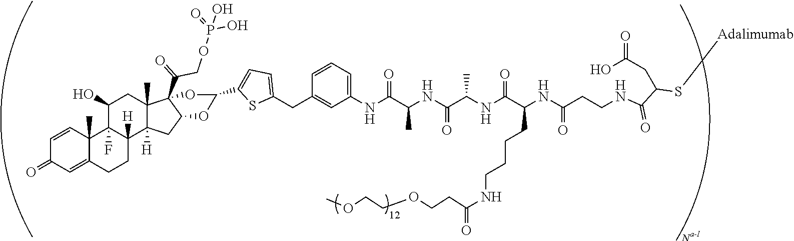 |
| II-C- 281 | 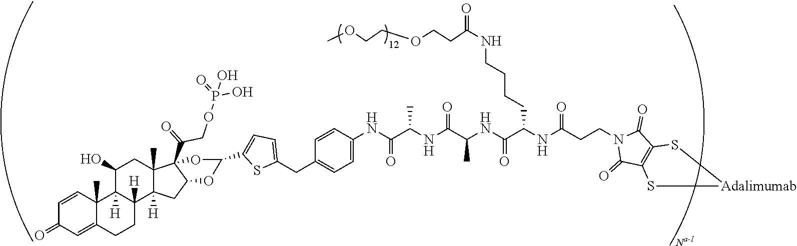 |
| II-C- 282 | 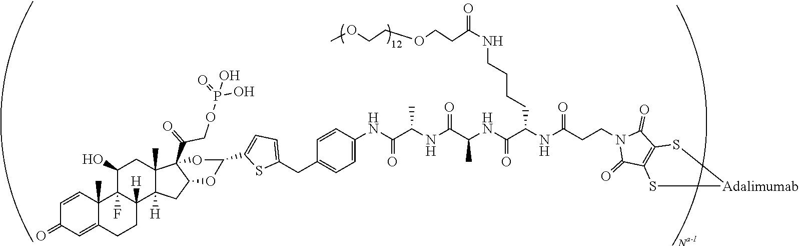 |
| II-C- 283 | 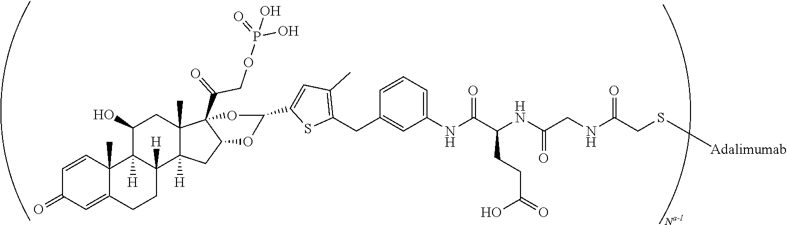 |
| II-C- 286 | 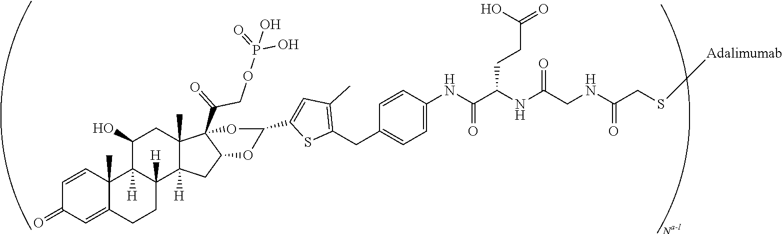 |
| II-C- 289 | 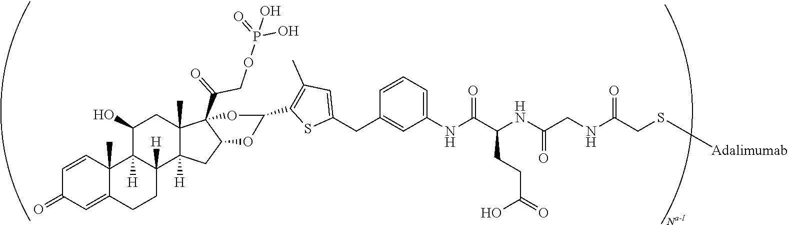 |
| II-C- 292 | 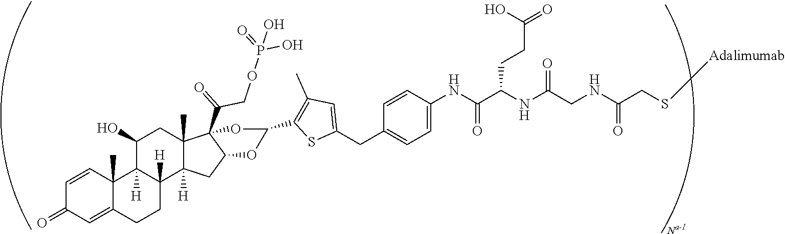 |
| II-C- 295 | 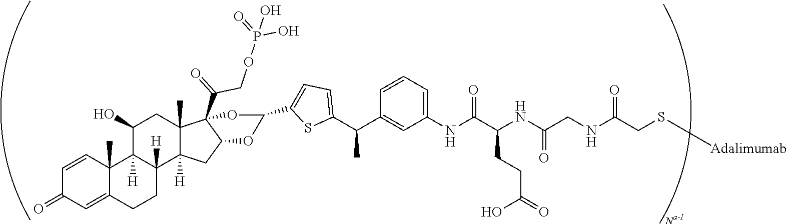 |
| II-C- 298 | 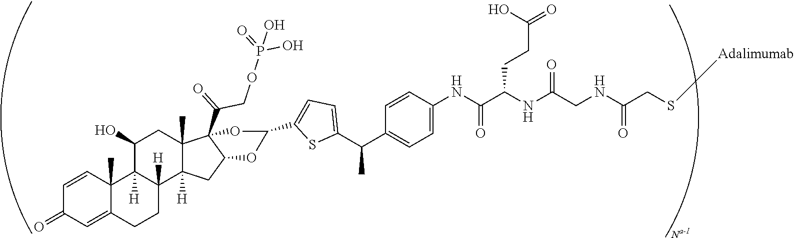 |
| II-C- 301 | 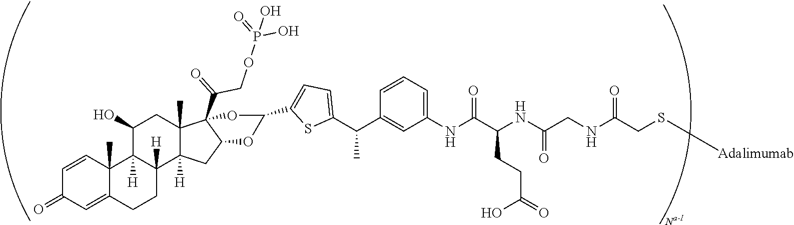 |
| II-C- 304 | 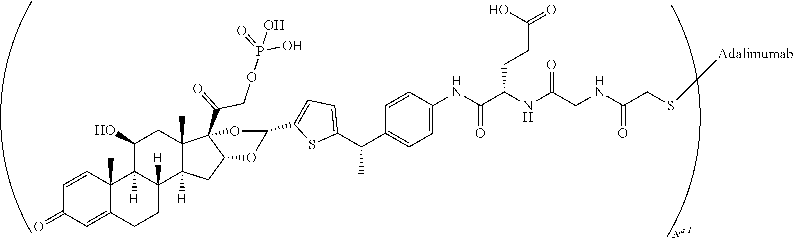 |
| II-C- 307 | 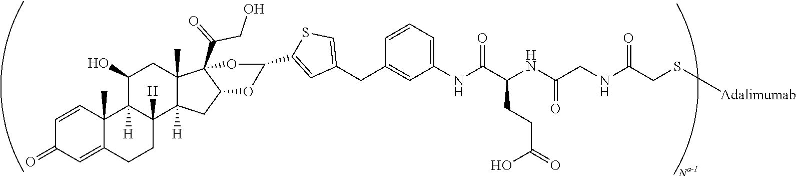 |
| II-C- 308 | 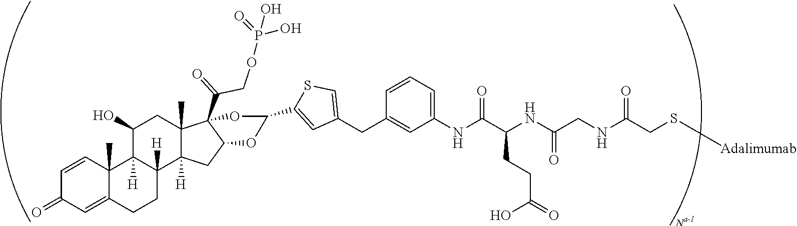 |
| II-C- 312 | 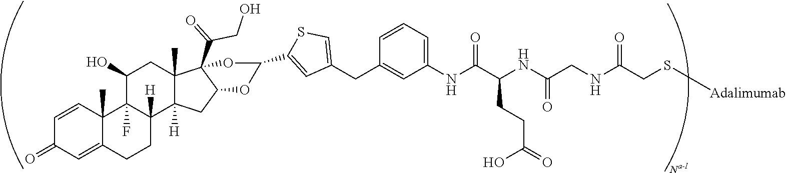 |
| II-C- 313 | 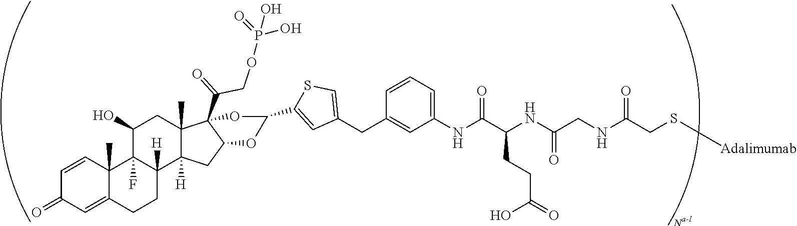 |
| II-C- 317 | 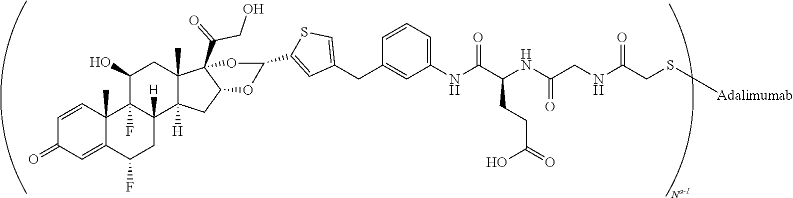 |
| II-C- 318 | 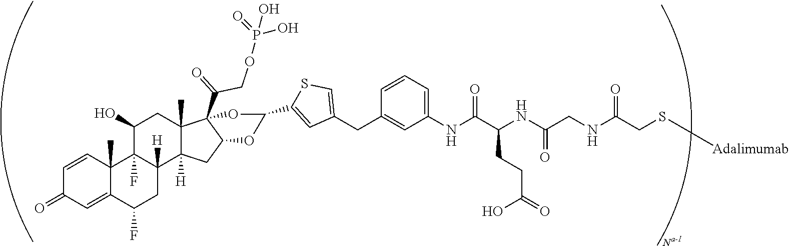 |
| II-C- 322 | 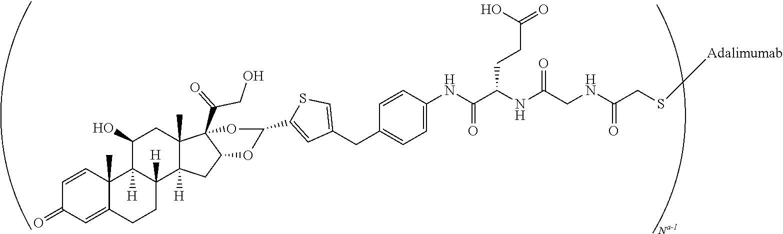 |
| II-C- 323 | 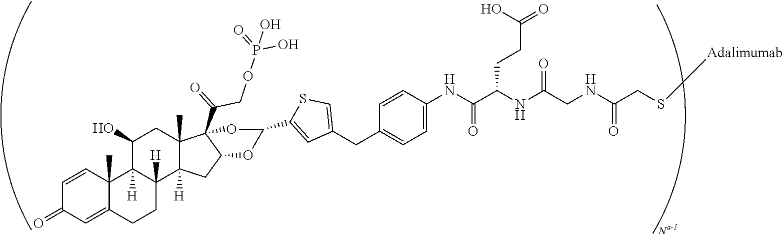 |
| II-C- 327 | 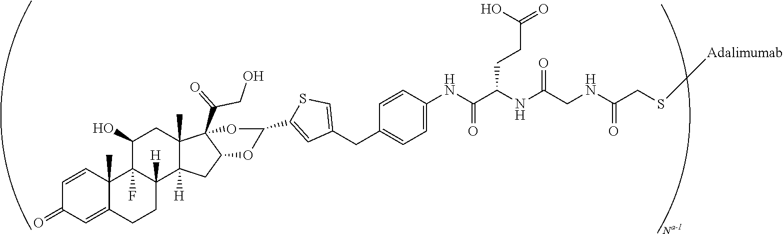 |
| II-C- 328 | 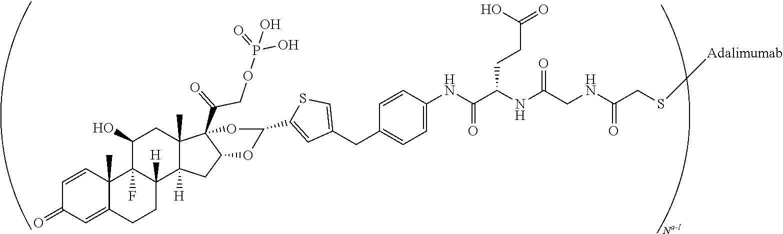 |
| II-C- 332 | 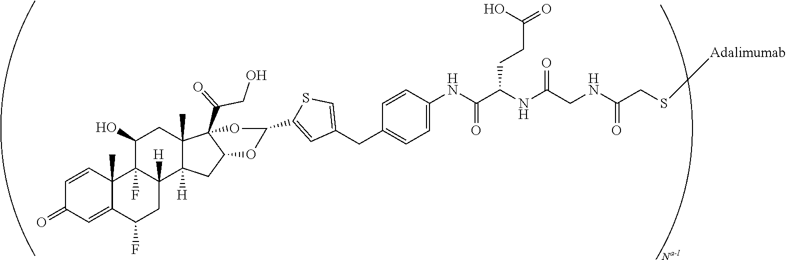 |
| II-C- 333 | 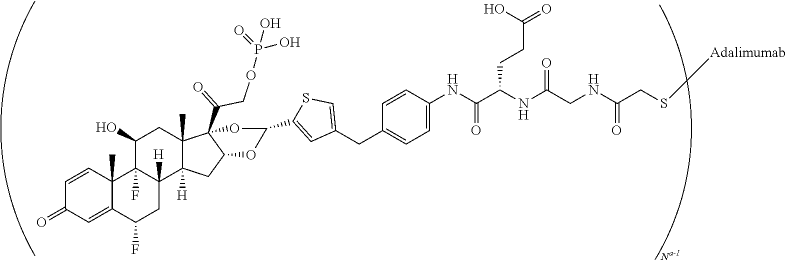 |
| II-C- 337 | 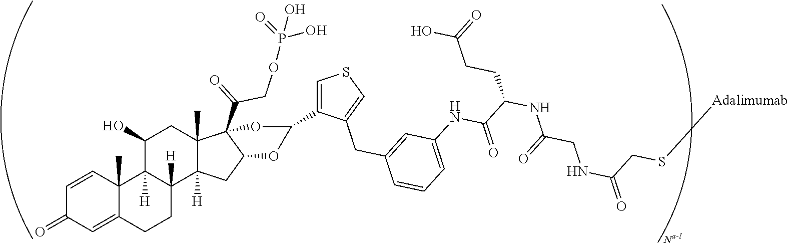 |
| II-C- 338 | 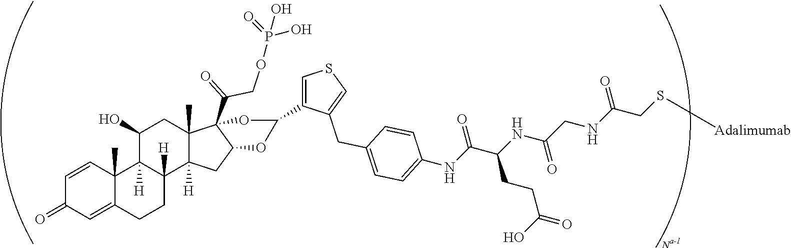 |
| II-C- 339 | 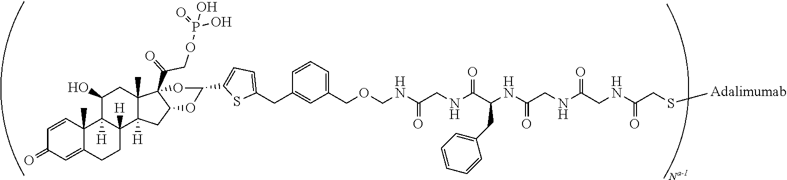 |
| II-C- 340 | 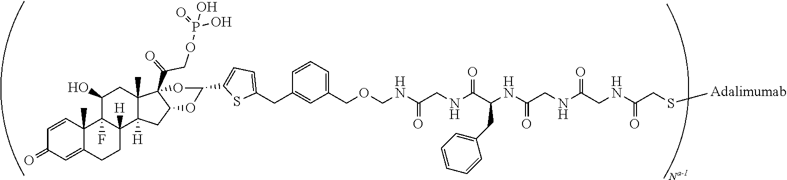 |
| II-C- 341 | 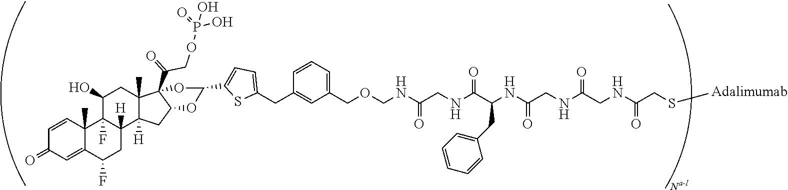 |
| II-C- 342 | 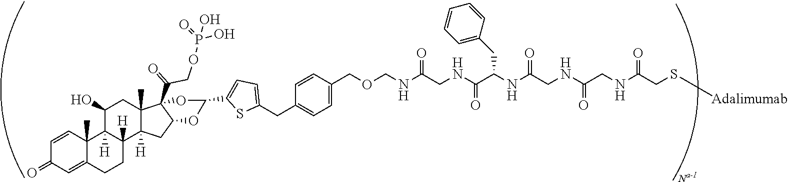 |
| II-C- 343 | 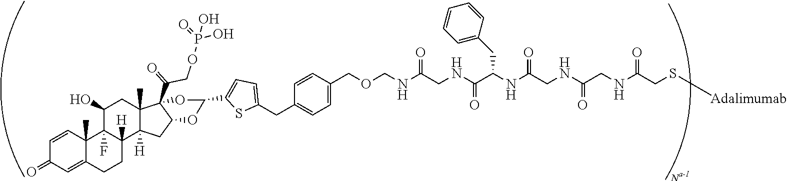 |
| II-C- 344 | 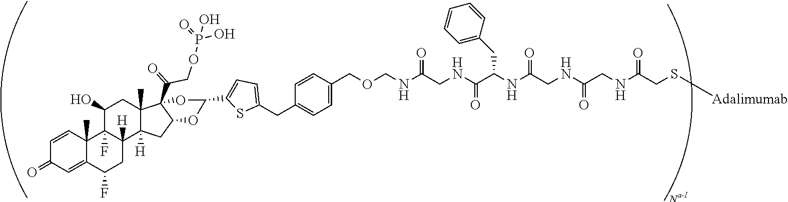 |
| II-C- 345 | 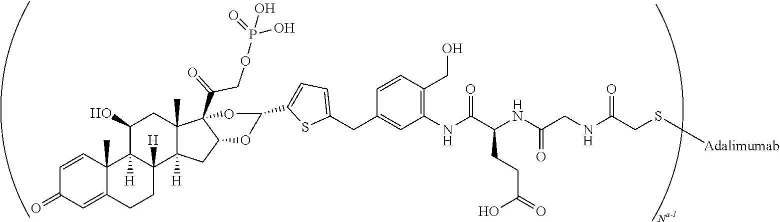 |
| II-C- 348 | 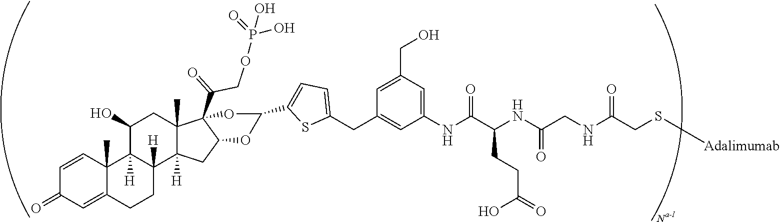 |
| II-C- 351 | 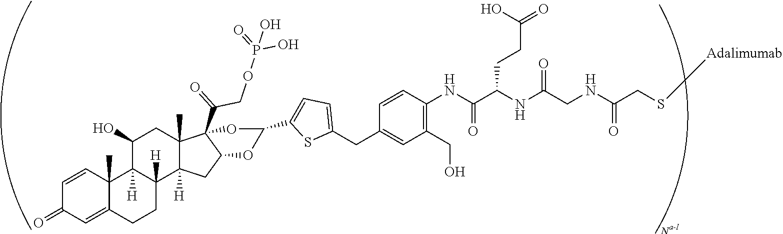 |
| II-C- 354 | 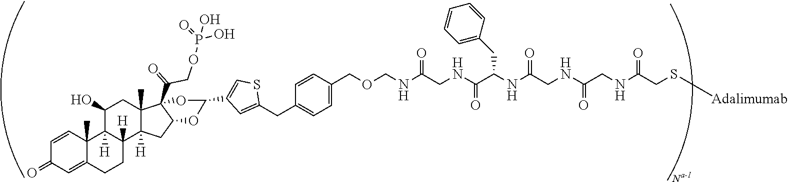 |
| II-C- 355 | 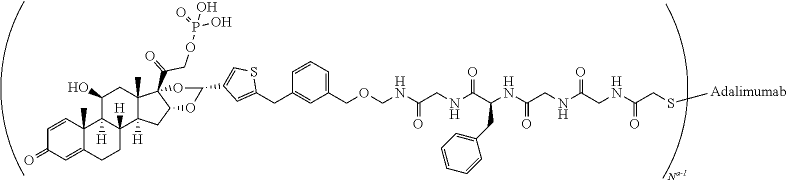 |
| II-C- 356 | 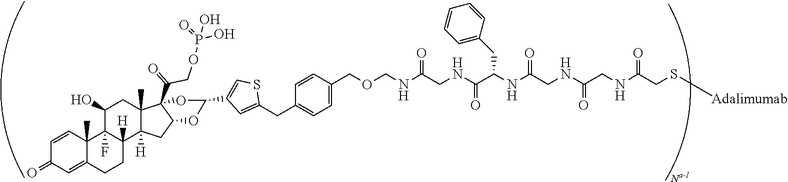 |
| II-C- 357 | 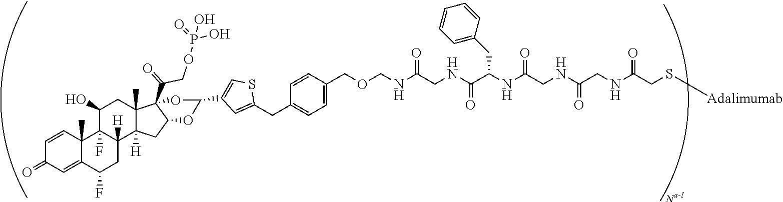 |
| II-C- 358 | 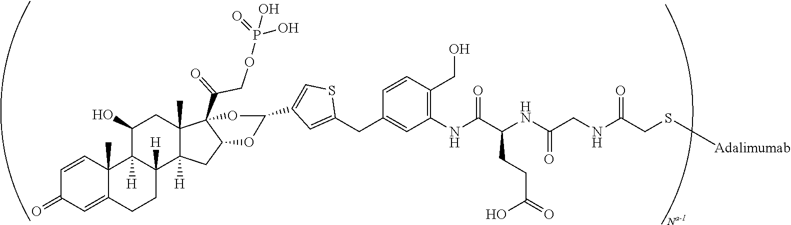 |
| II-C- 361 | 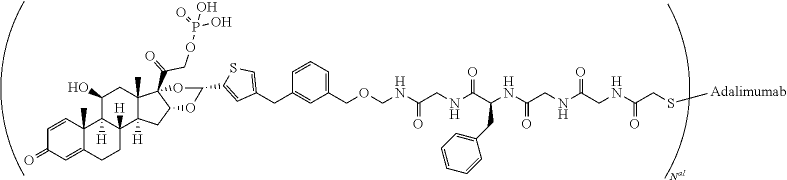 |
| II-C- 362 | 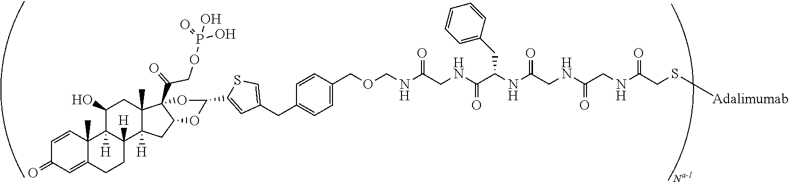 |
| II-C- 363 | 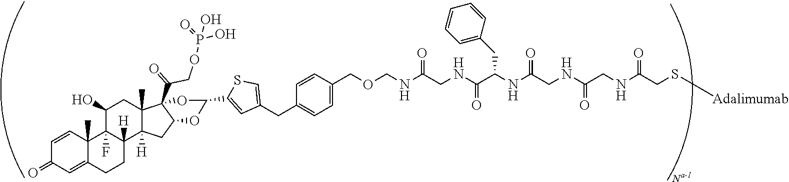 |
| II-C- 364 | 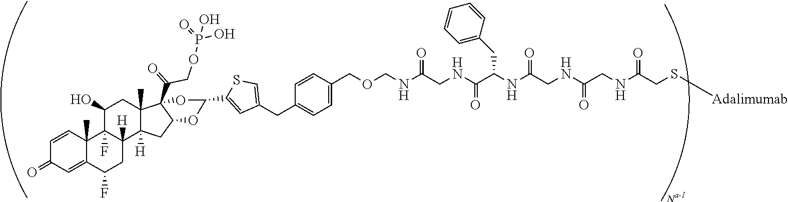 |
| II-C- 365 | 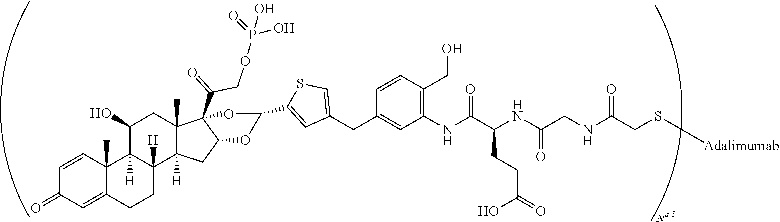 |
| II-C- 368 | 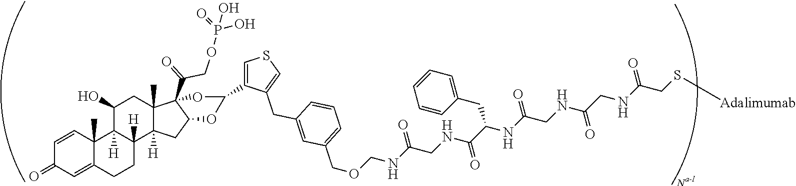 |
| II-C- 369 | 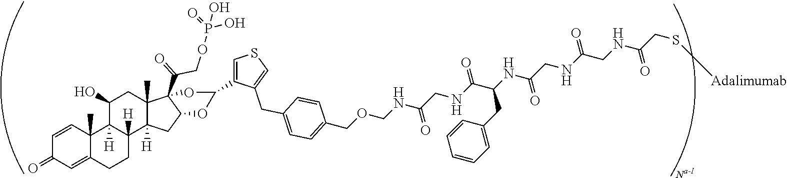 |
| II-C- 385 | 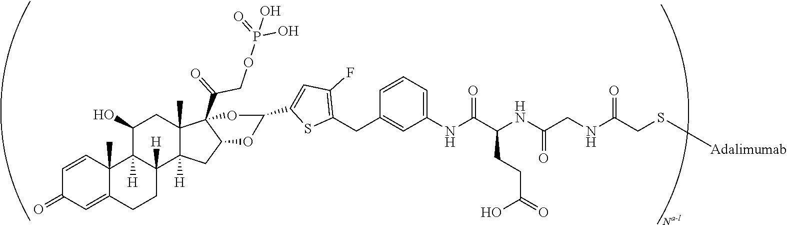 |
| II-C- 388 | 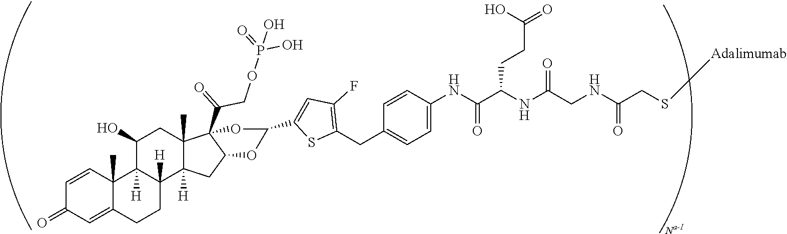 |
| II-C- 391 | 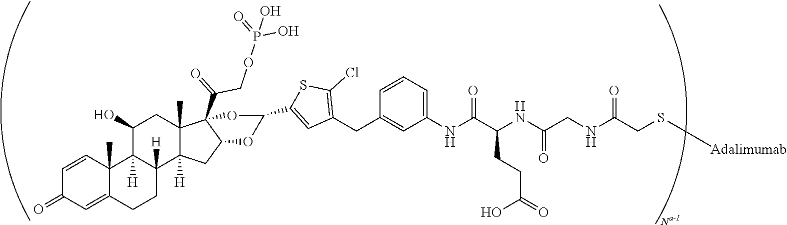 |
| II-C- 394 | 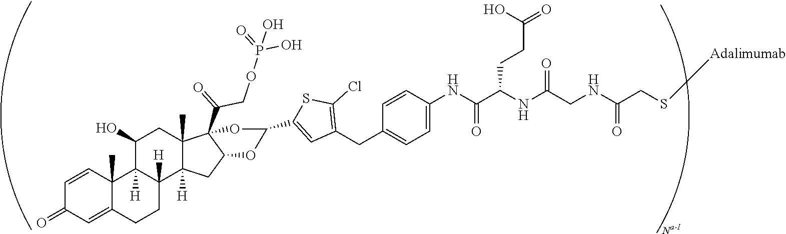 |
| II-C- 397 | 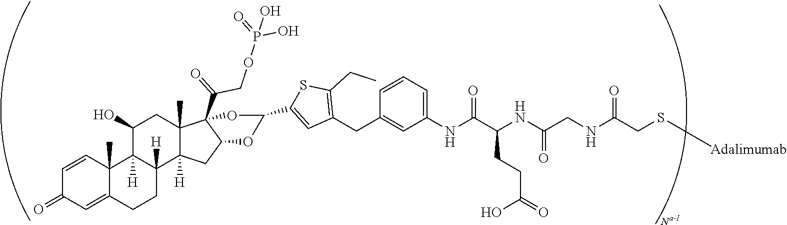 |
| II-C- 400 | 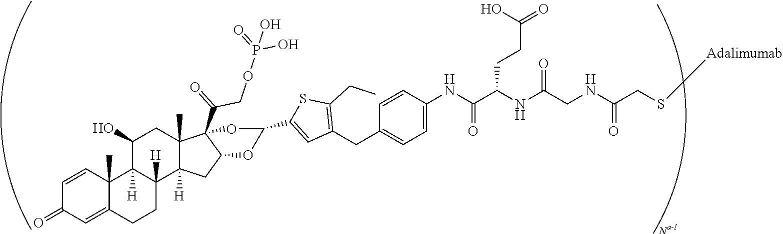 |
| II-C- 403 | 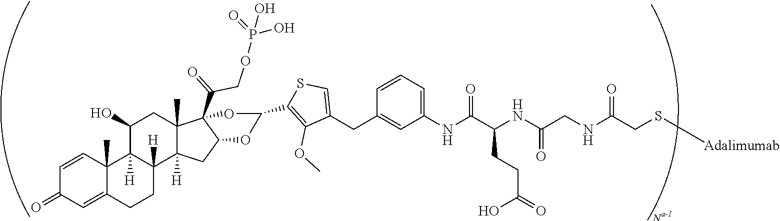 |
| II-C- 406 | 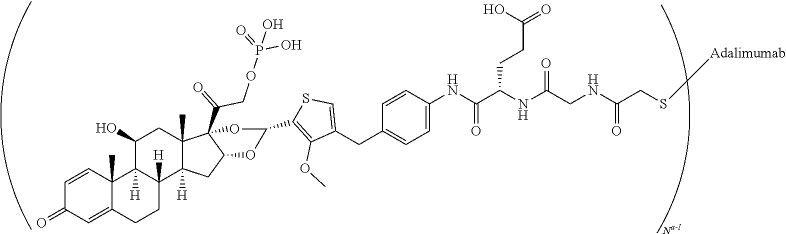 |
| II-C- 409 | 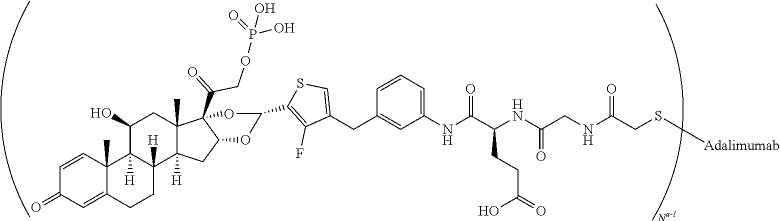 |
| II-C- 412 | 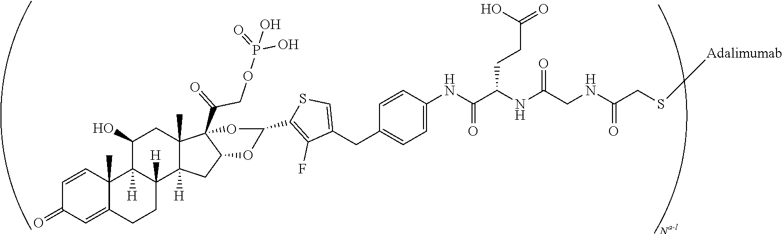 |
| II-C- 415 | 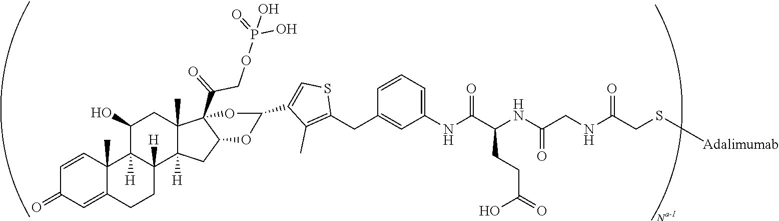 |
| II-C- 418 | 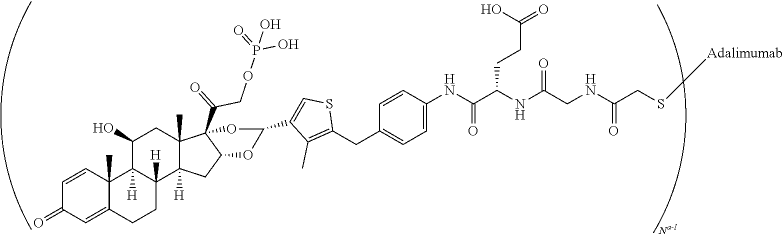 |
| II-C- 421 | 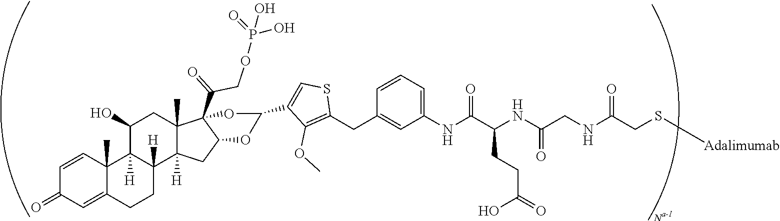 |
| II-C- 424 | 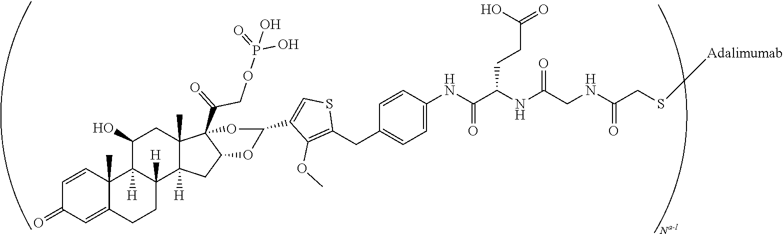 |
| II-C- 427 | 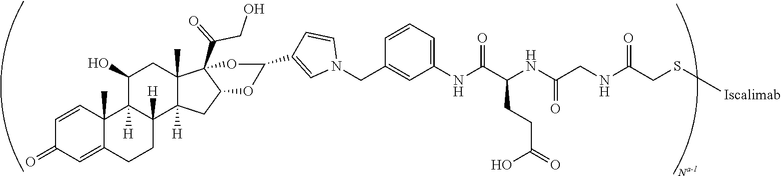 |
| II-C- 428 | 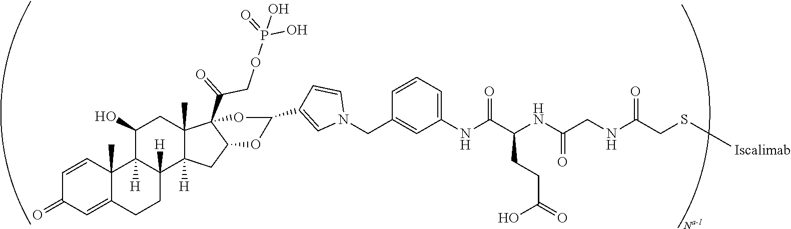 |
| II-C- 433 | 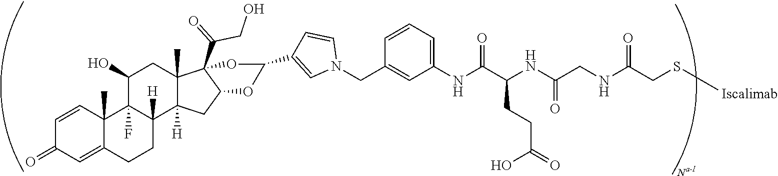 |
| II-C- 434 | 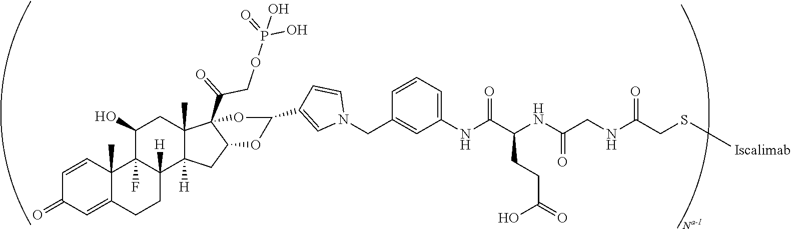 |
| II-C- 438 | 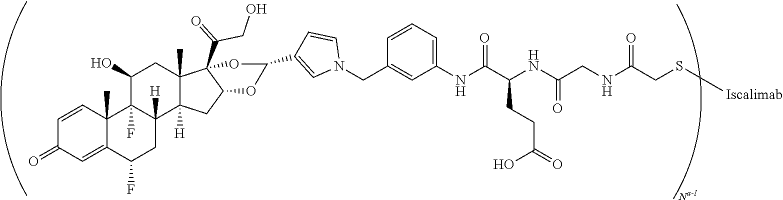 |
| II-C- 439 | 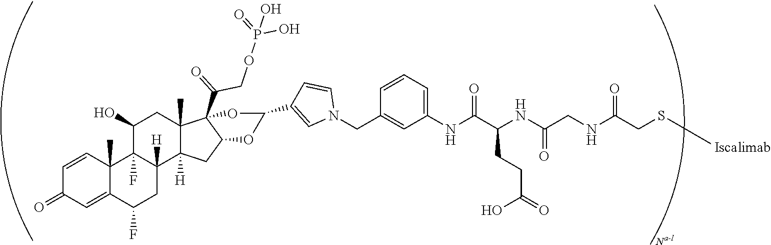 |
| II-C- 443 | 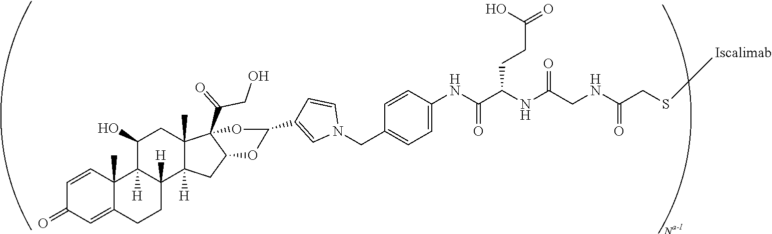 |
| II-C- 444 | 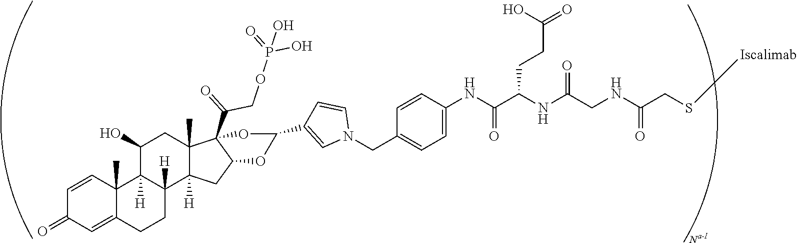 |
| II-C- 449 | 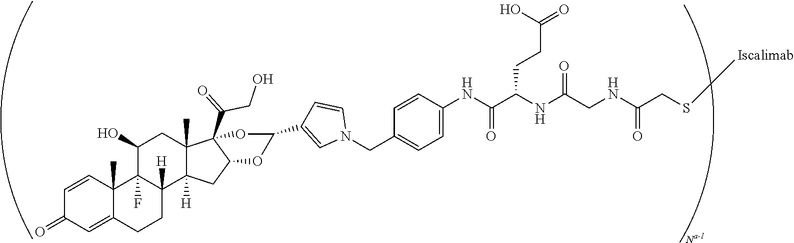 |
| II-C- 450 | 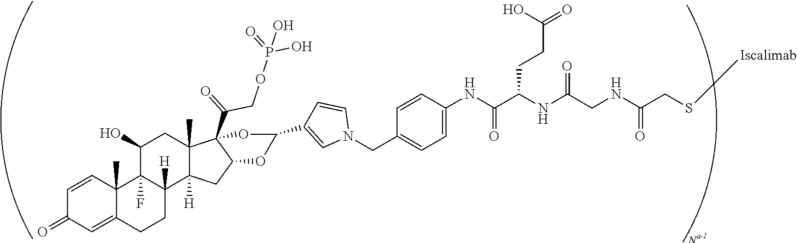 |
| II-C- 455 | 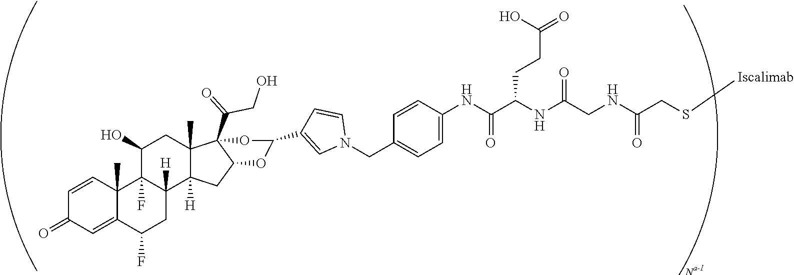 |
| II-C- 456 | 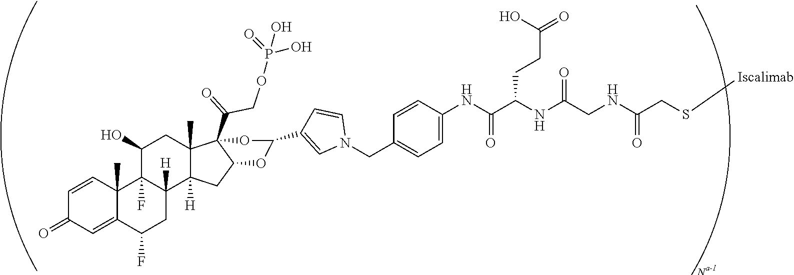 |
| II-C- 460 | 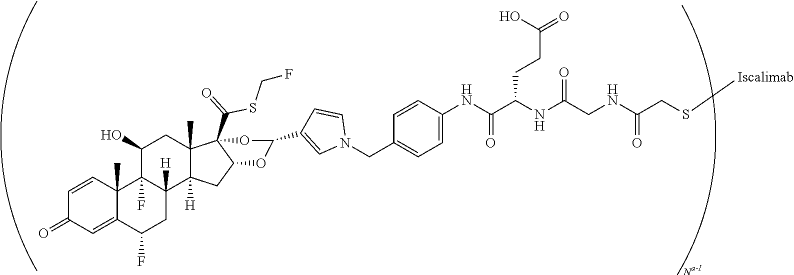 |
| II-C- 461 | 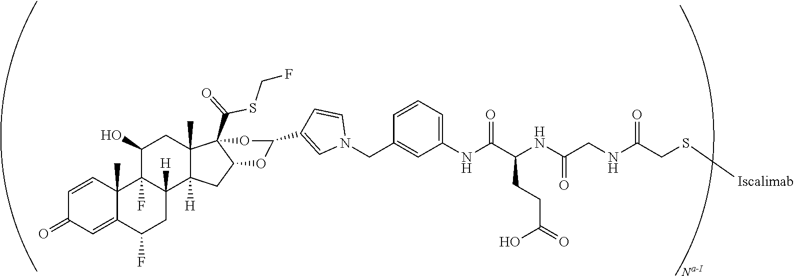 |
| II-C- 537 | 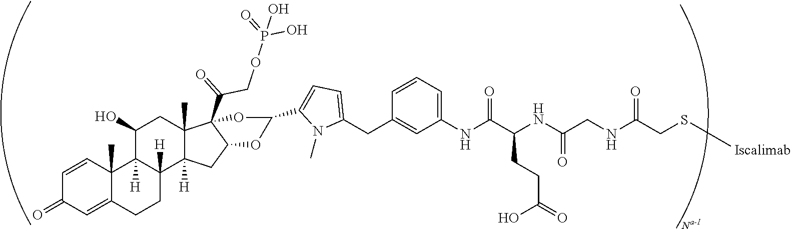 |
| II-C- 540 | 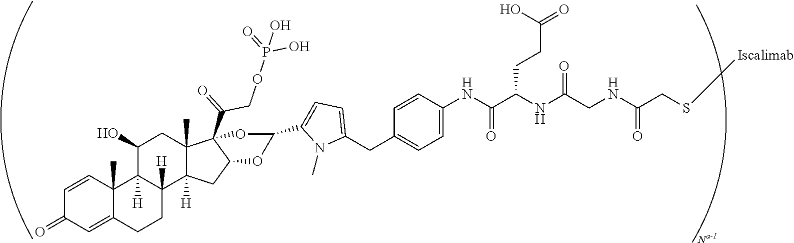 |
| II-C- 543 | 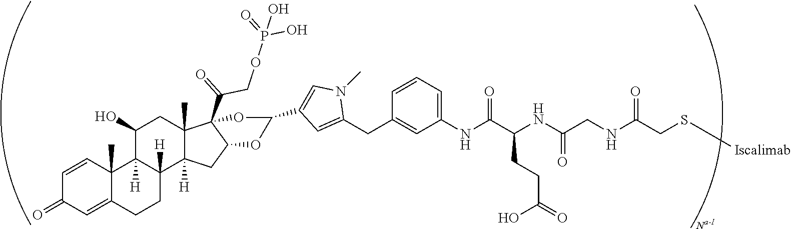 |
| II-C- 546 | 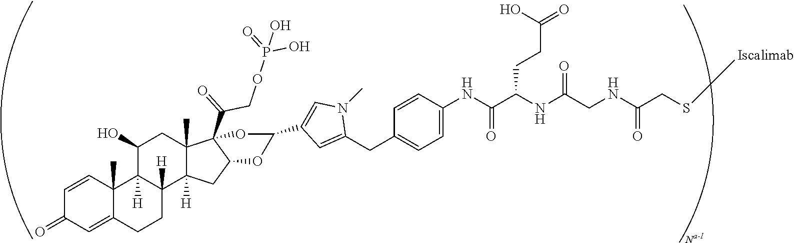 |
| II-C- 549 | 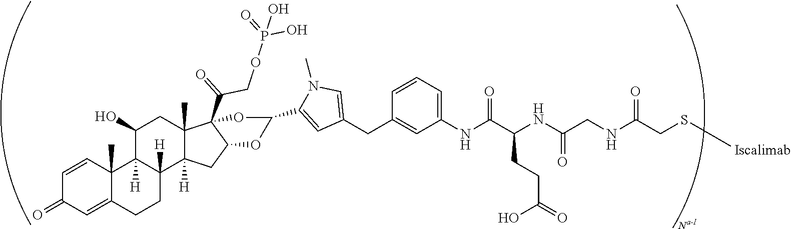 |
| II-C- 552 | 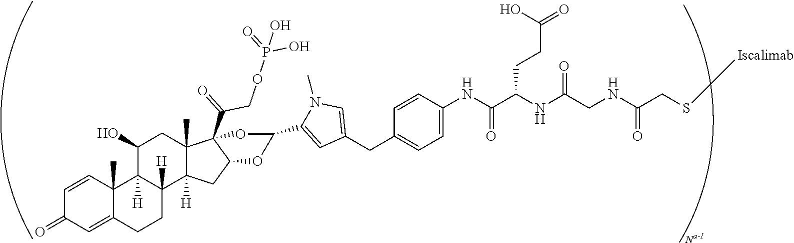 |
| II-C- 592 | 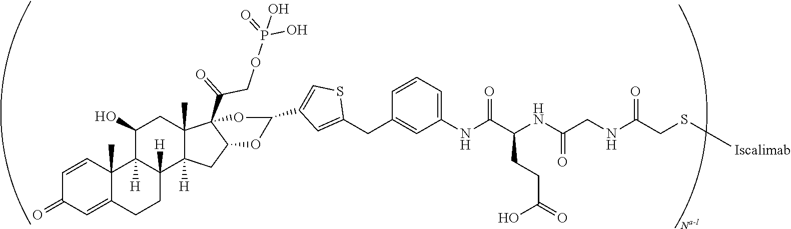 |
| II-C- 597 | 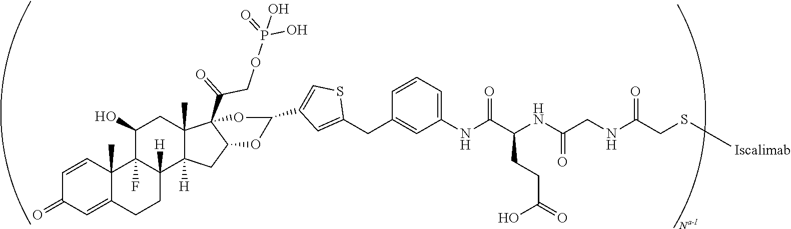 |
| II-C- 602 | 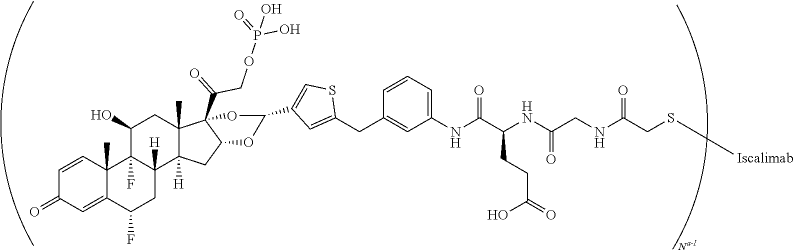 |
| II-C- 607 | 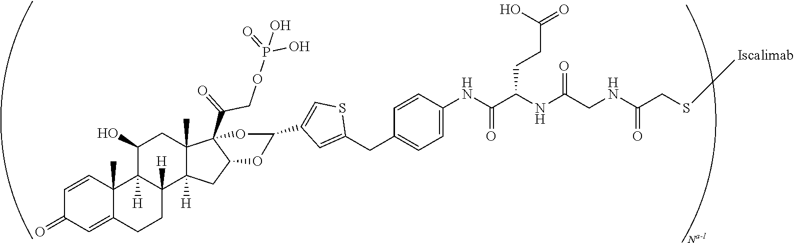 |
| II-C- 612 | 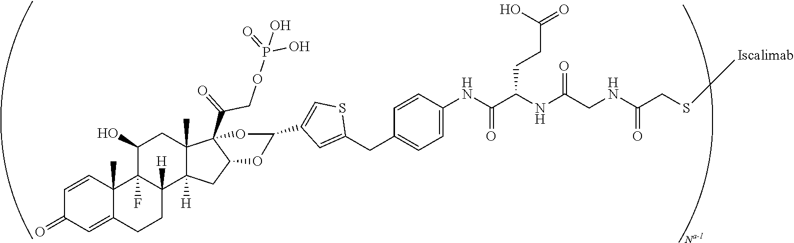 |
| II-C- 617 | 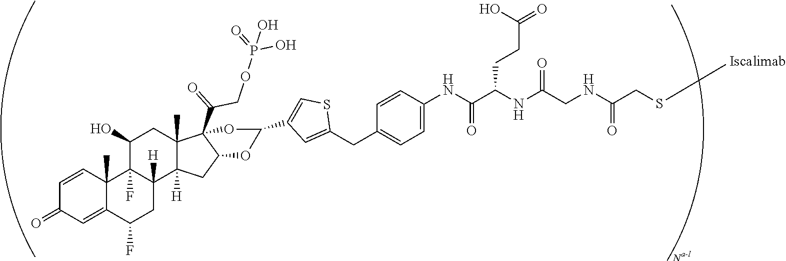 |
| II-C- 622 | 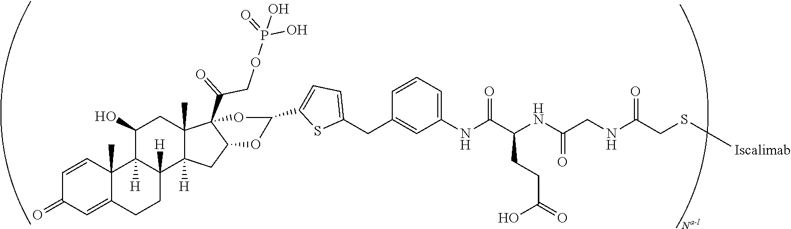 |
| II-C- 627 | 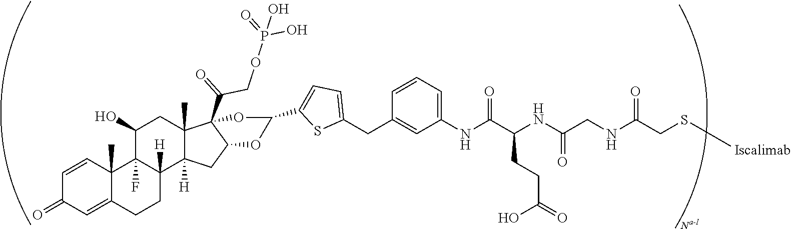 |
| II-C- 632 | 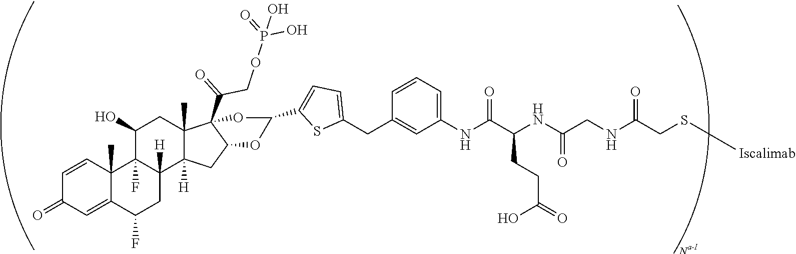 |
| II-C- 637 | 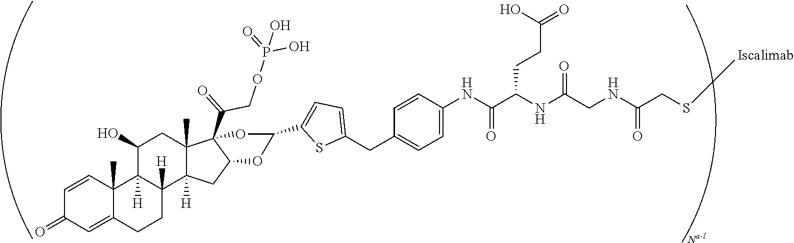 |
| II-C- 642 | 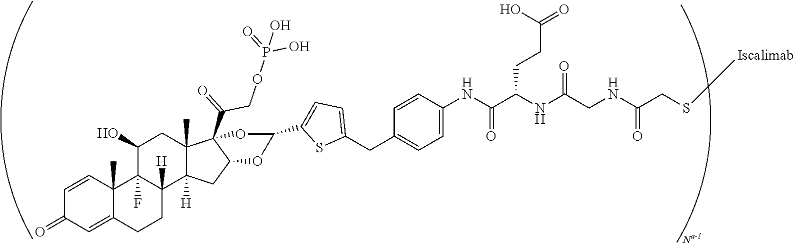 |
| II-C- 647 | 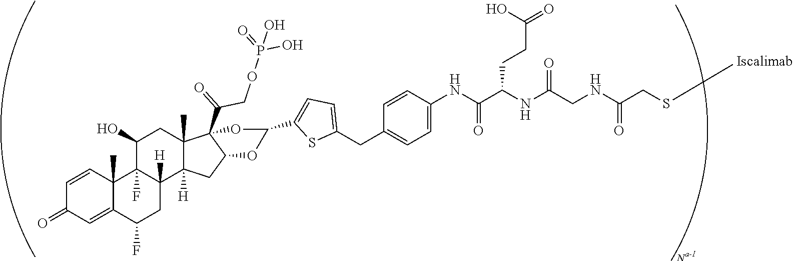 |
| II-C- 659 | 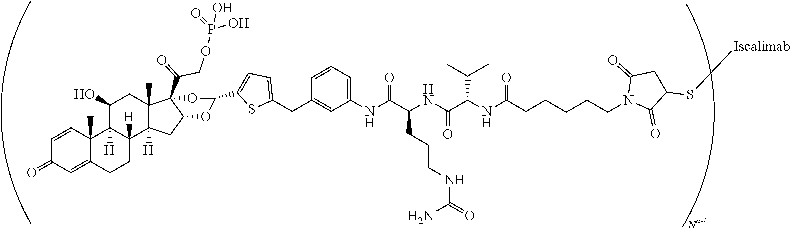 |
| II-C- 661 | 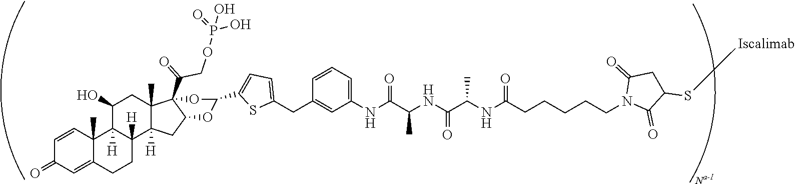 |
| II-C- 663 | 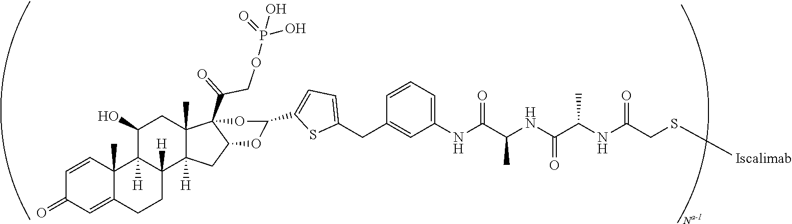 |
| II-C- 712 | 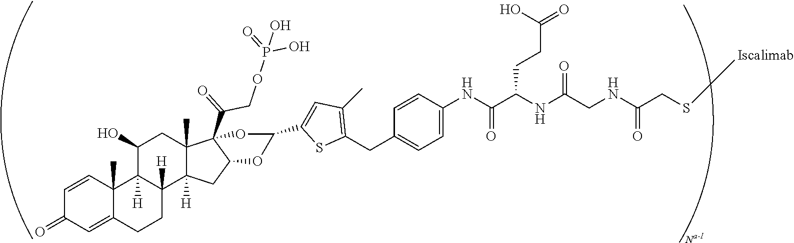 |
| II-C- 715 | 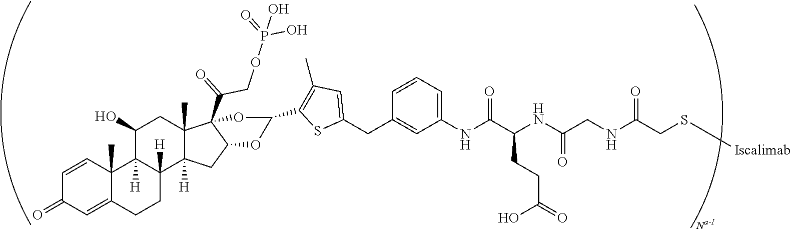 |
| II-C- 724 | 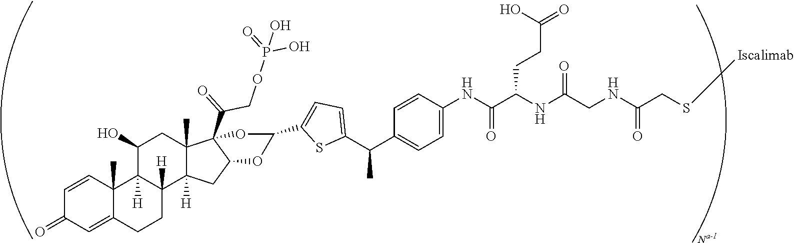 |
| II-C- 727 | 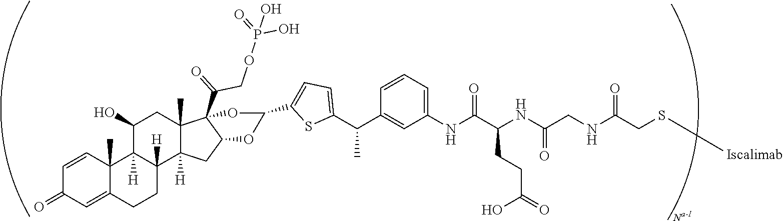 |
| II-C- 734 | 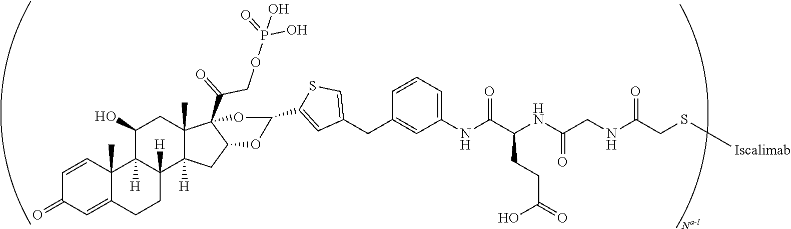 |
| II-C- 739 | 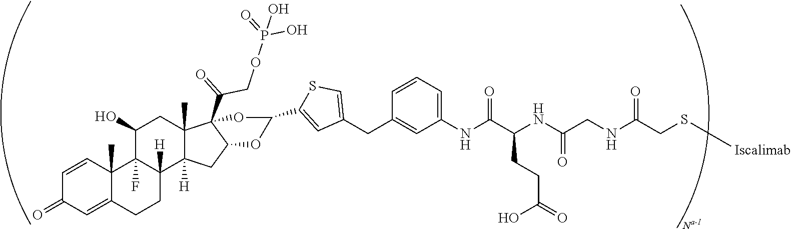 |
| II-C- 744 | 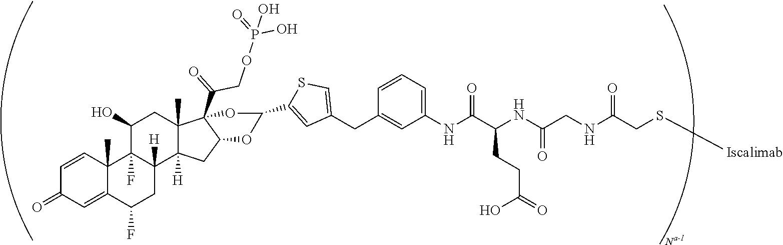 |
| II-C- 749 | 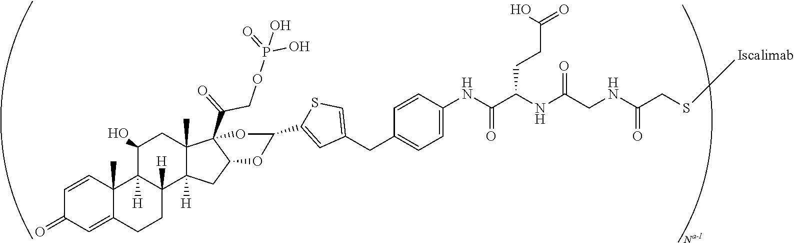 |
| II-C- 754 | 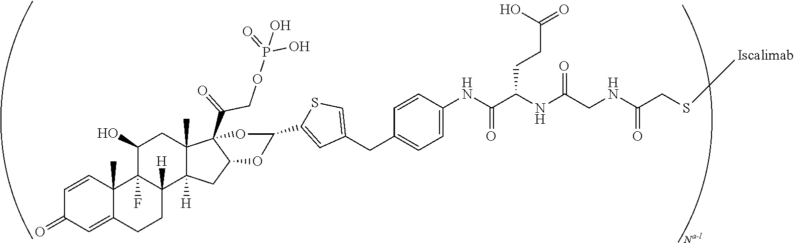 |
| II-C- 758 | 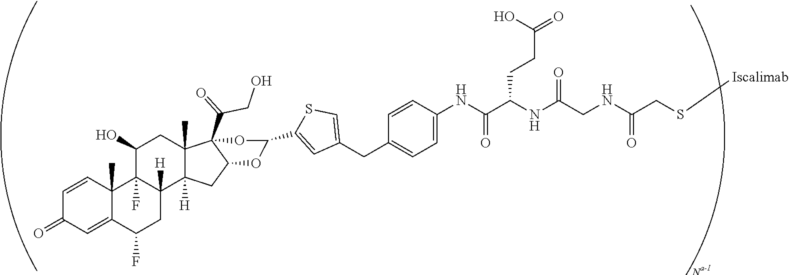 |
| II-C- 759 | 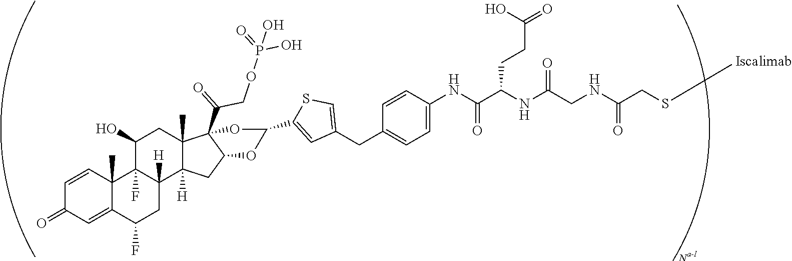 |
| II-C- 763 | 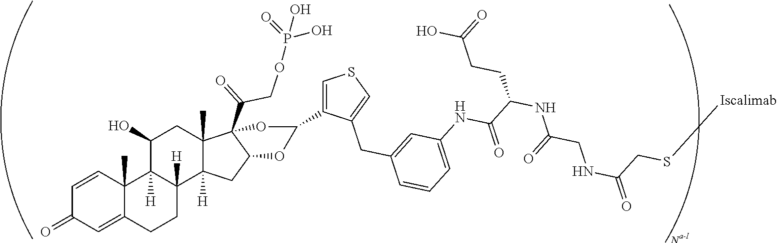 |
| II-C- 764 | 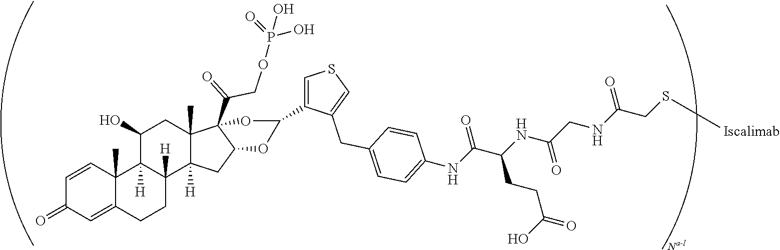 |
| II-C- 765 | 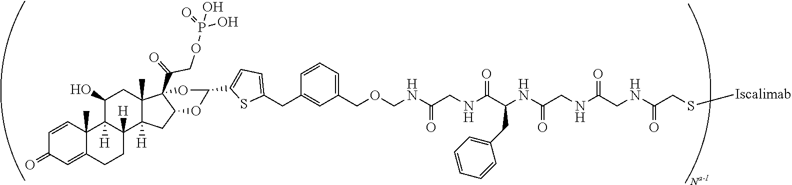 |
| II-C- 766 | 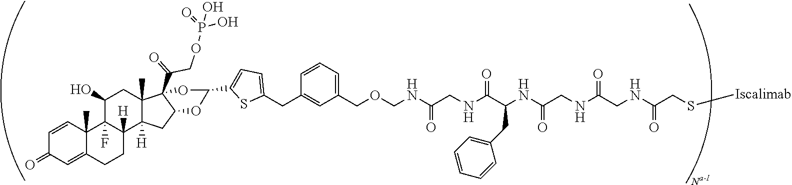 |
| II-C- 767 | 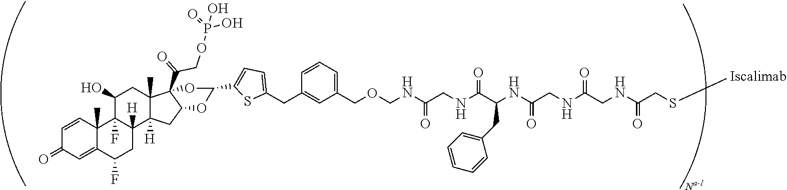 |
| II-C- 768 | 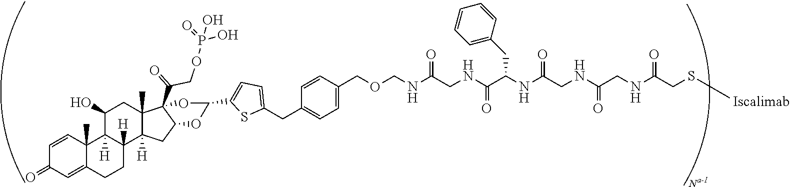 |
| II-C- 769 | 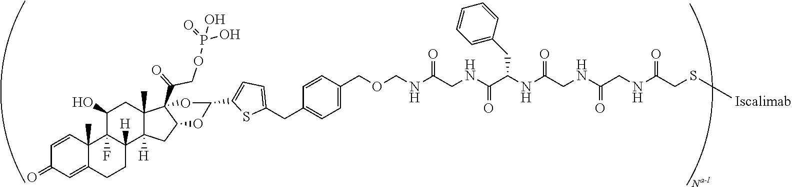 |
| II-C- 770 | 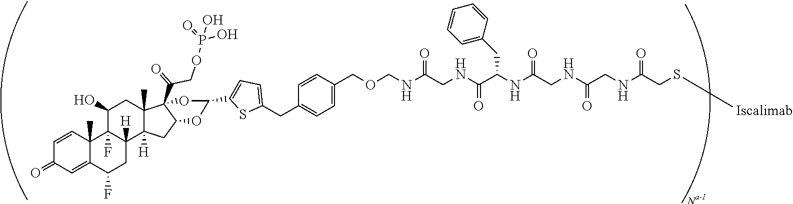 |
| II-C- 771 | 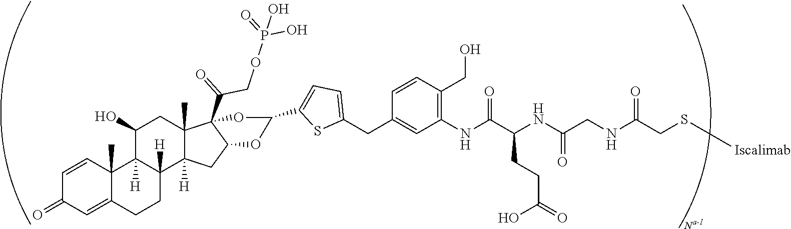 |
| II-C- 774 | 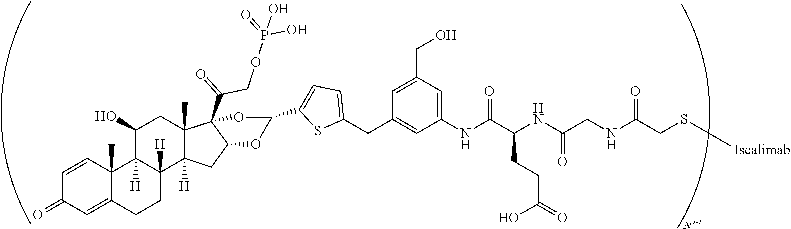 |
| II-C- 777 | 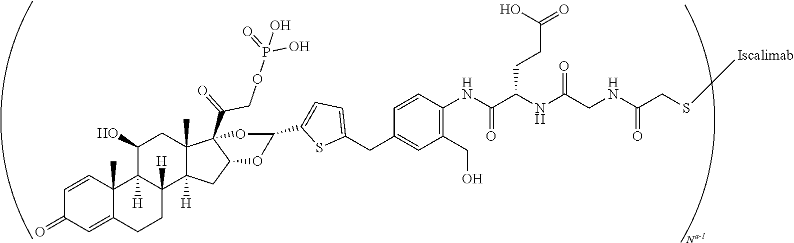 |
| II-C- 780 | 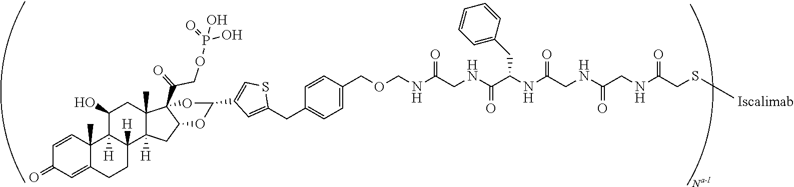 |
| II-C- 781 | 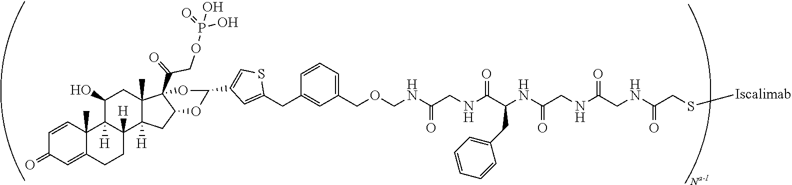 |
| II-C- 782 | 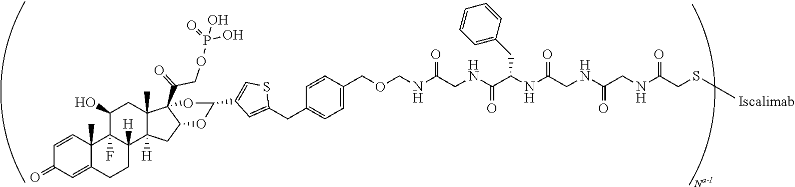 |
| II-C- 783 | 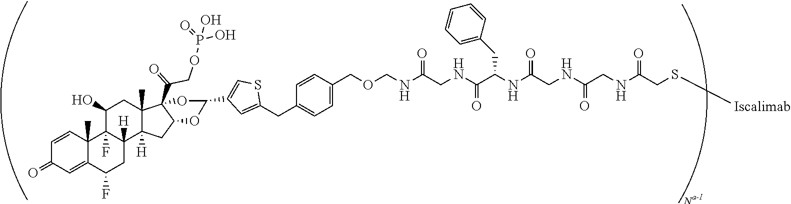 |
| II-C- 784 | 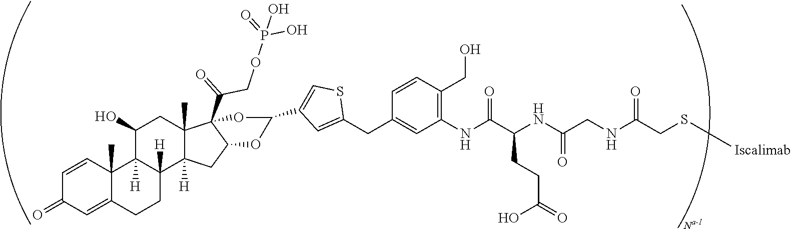 |
| II-C- 787 | 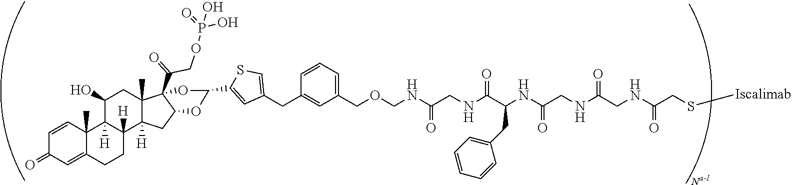 |
| II-C- 788 | 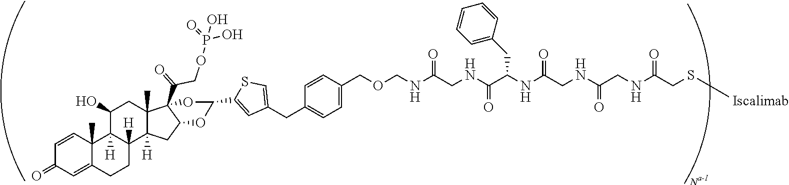 |
| II-C- 789 | 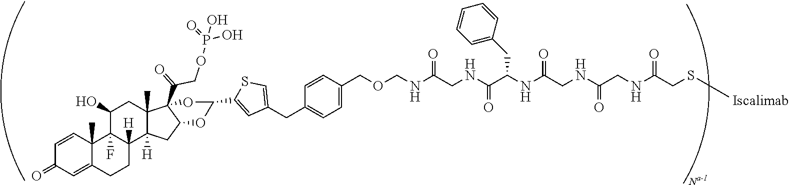 |
| II-C- 790 | 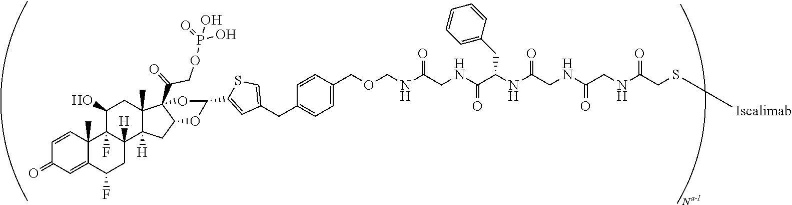 |
| II-C- 791 | 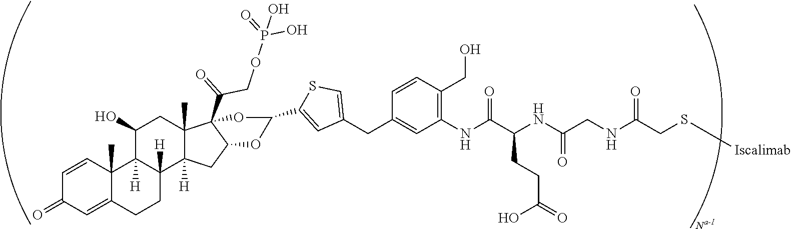 |
| II-C- 794 | 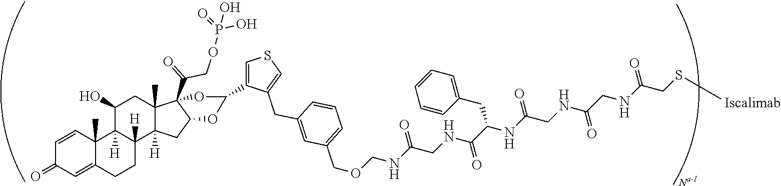 |
| II-C- 795 | 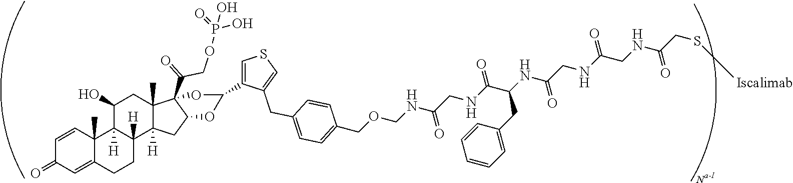 |
| II-C- 814 | 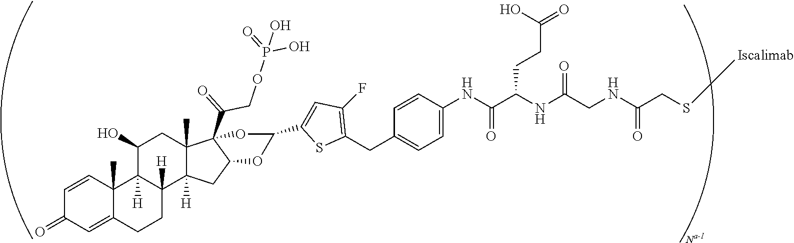 |
| II-C- 817 | 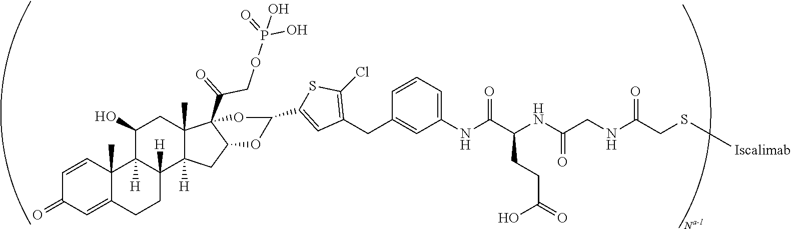 |
| II-C- 826 | 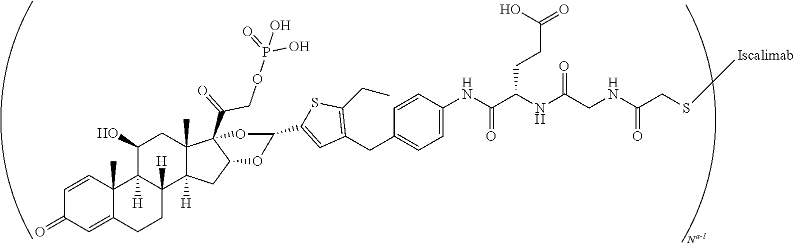 |
| II-C- 829 | 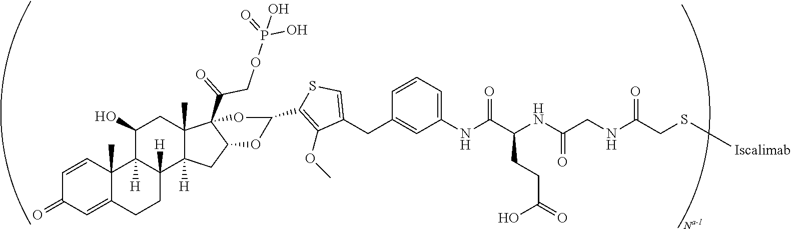 |
| II-C- 838 | 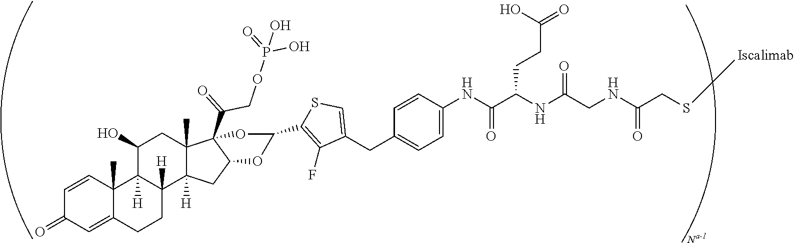 |
| II-C- 841 | 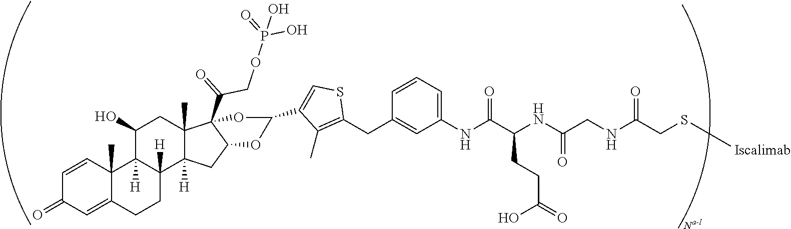 |
| II-C- 850 | 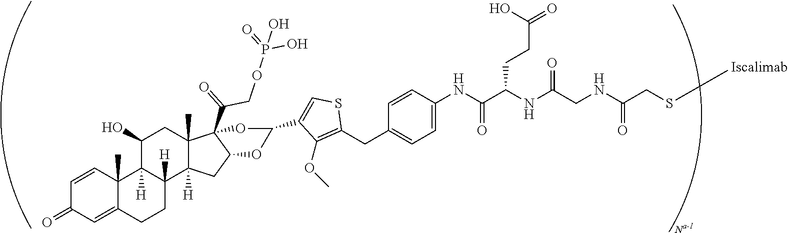 |
| II-C- 853 | 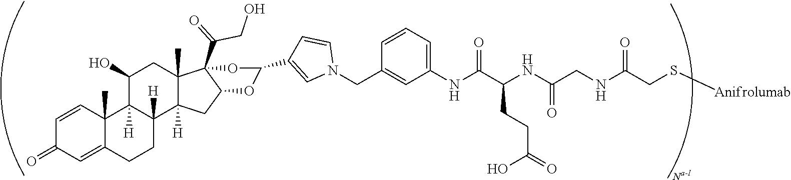 |
| II-C- 854 | 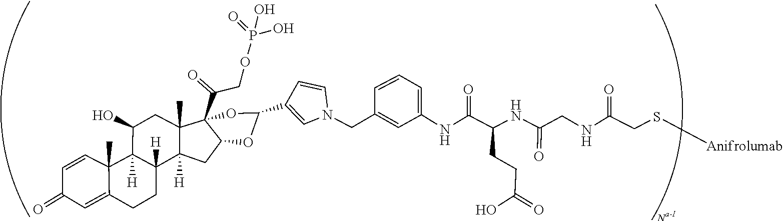 |
| II-C- 859 | 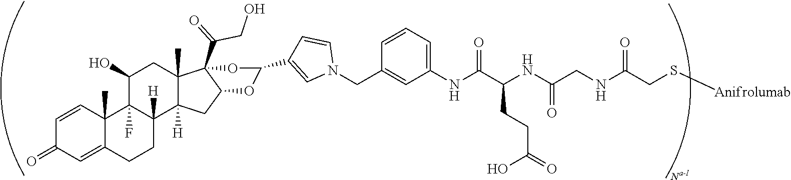 |
| II-C- 860 | 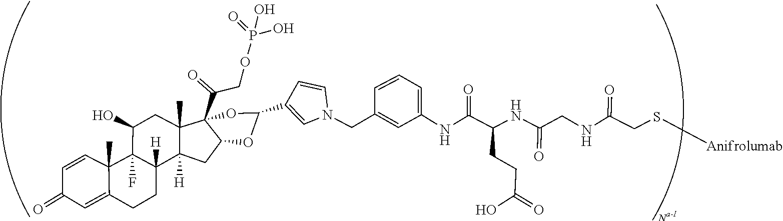 |
| II-C- 864 | 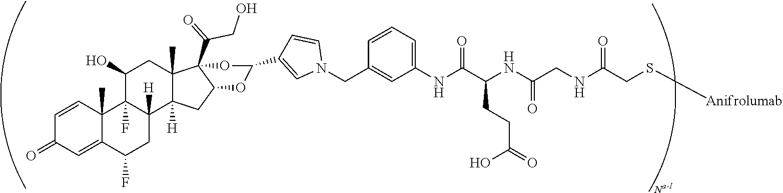 |
| II-C- 865 | 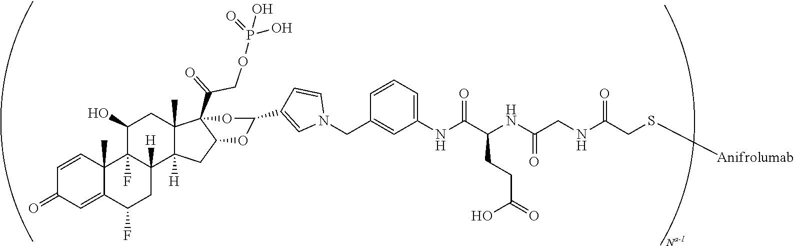 |
| II-C- 869 | 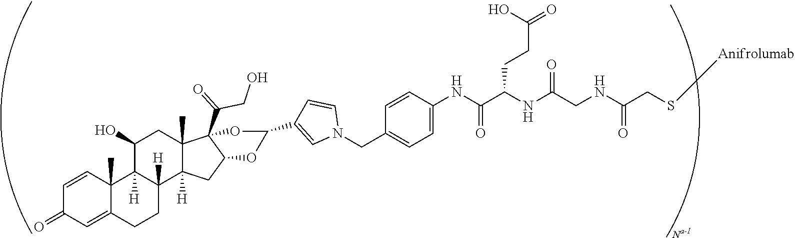 |
| II-C- 870 | 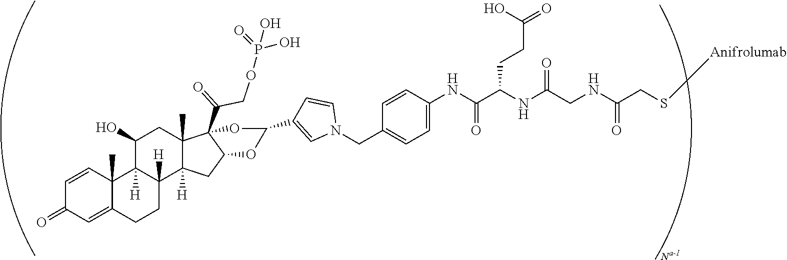 |
| II-C- 875 | 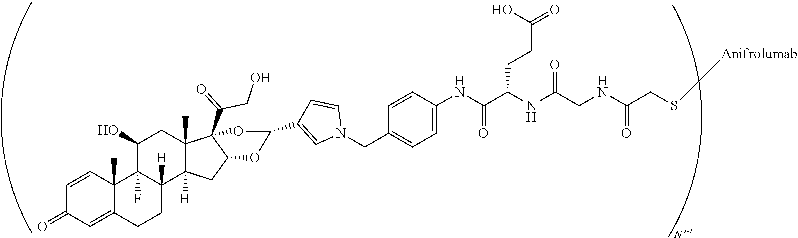 |
| II-C- 876 | 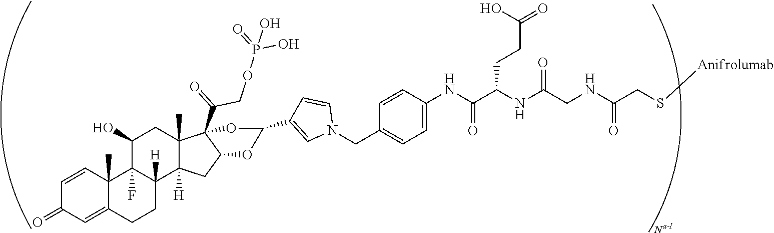 |
| II-C- 881 | 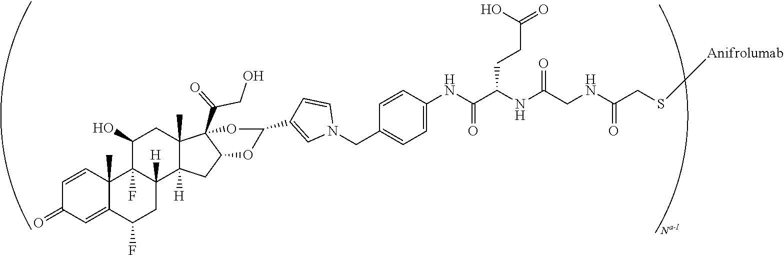 |
| II-C- 882 | 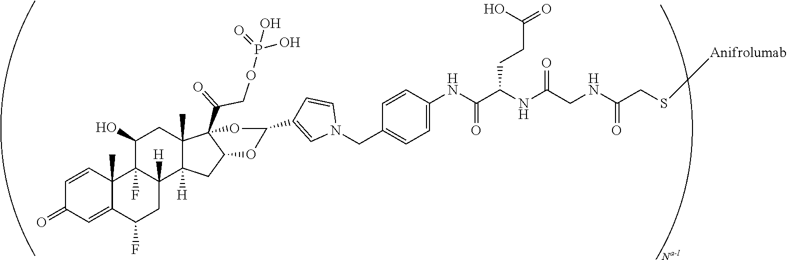 |
| II-C- 886 | 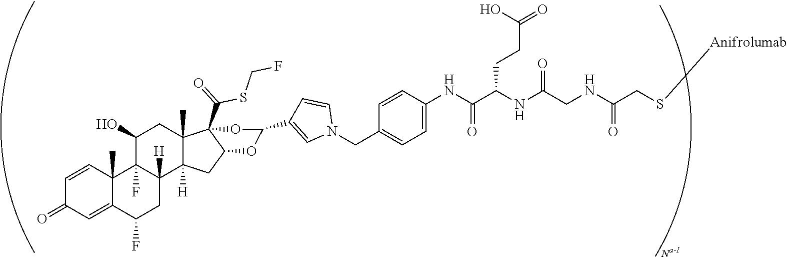 |
| II-C- 887 | 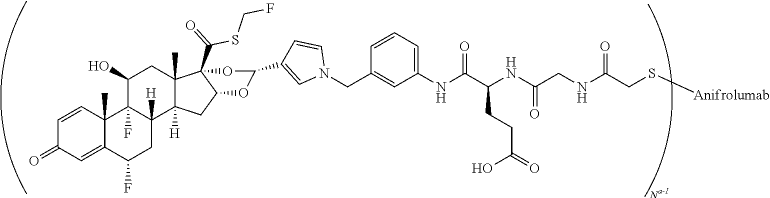 |
| II-C- 963 | 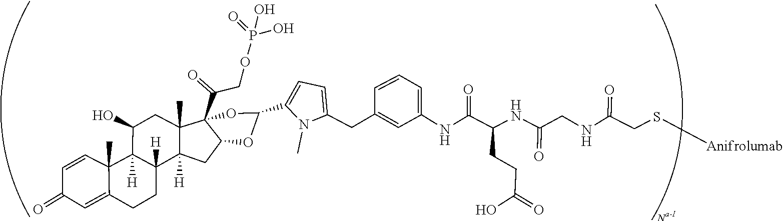 |
| II-C- 966 | 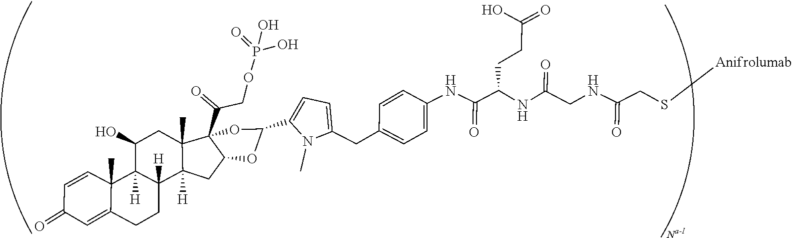 |
| II-C- 969 | 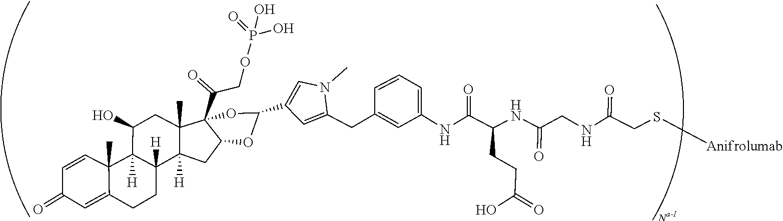 |
| II-C- 972 | 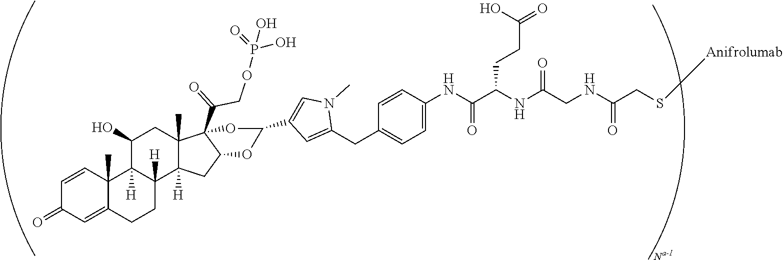 |
| II-C- 975 | 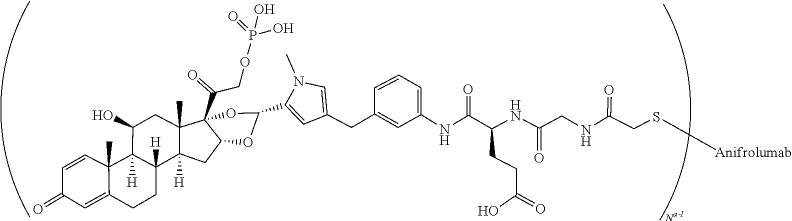 |
| II-C- 978 | 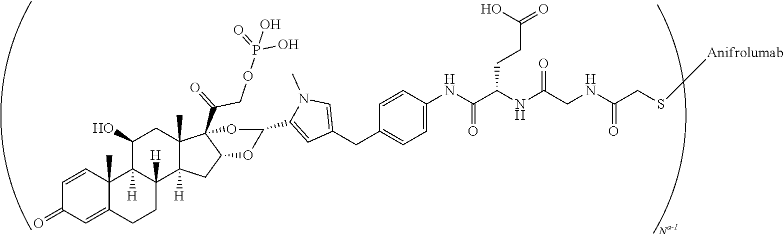 |
| II-C- 1017 | 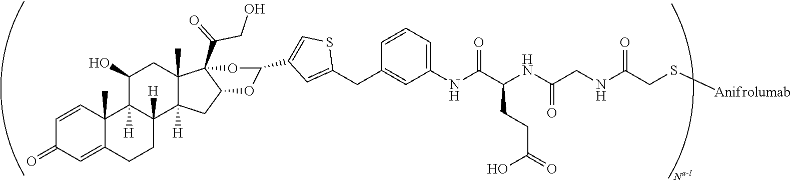 |
| II-C- 1018 | 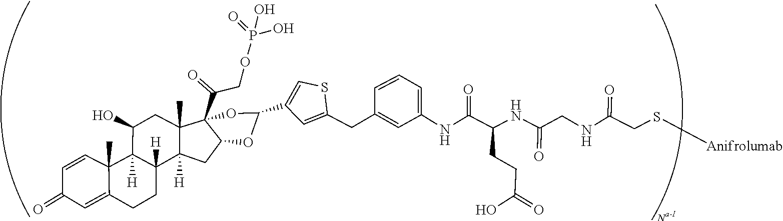 |
| II-C- 1022 | 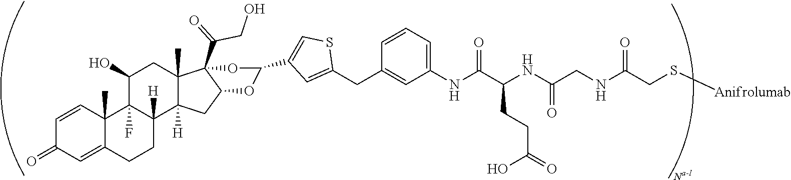 |
| II-C- 1023 | 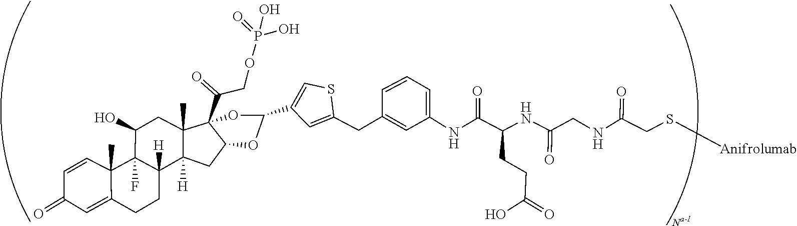 |
| II-C- 1027 | 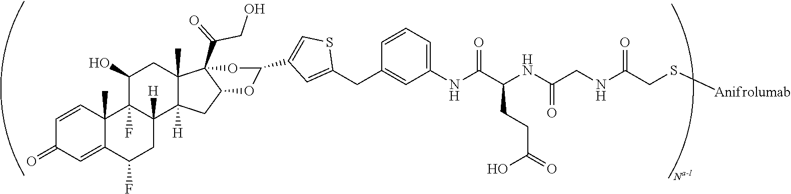 |
| II-C- 1028 | 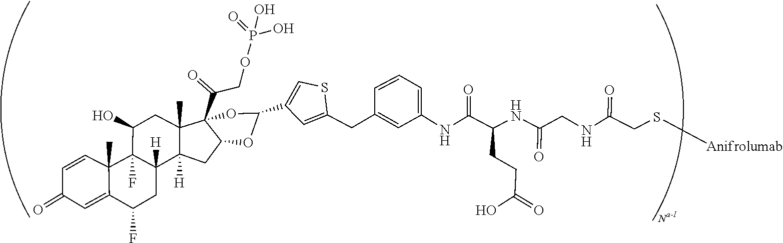 |
| II-C- 1032 | 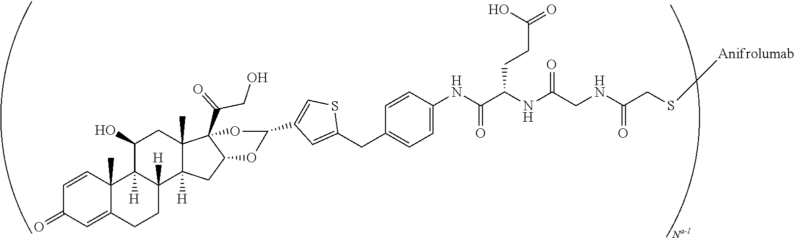 |
| II-C- 1033 | 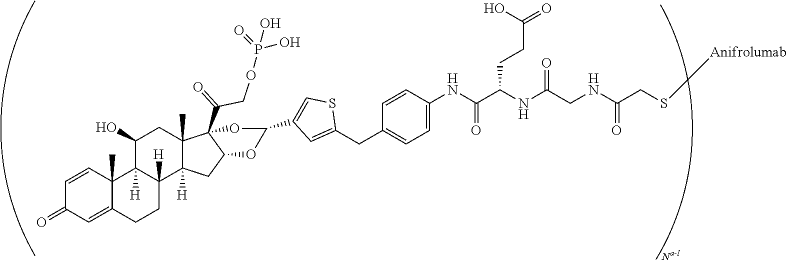 |
| II-C- 1037 | 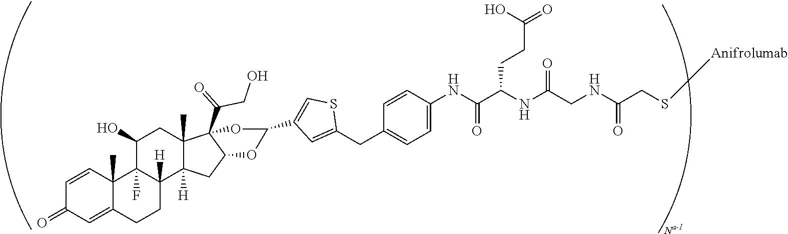 |
| II-C- 1038 | 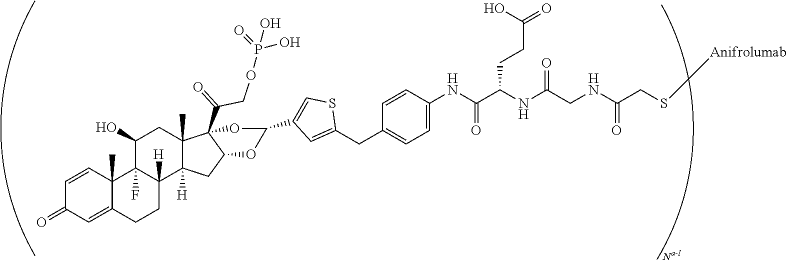 |
| II-C- 1042 | 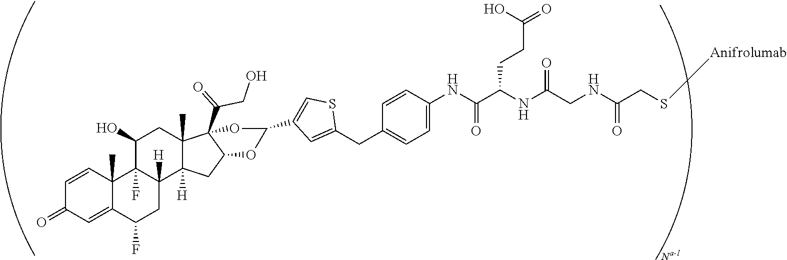 |
| II-C- 1043 | 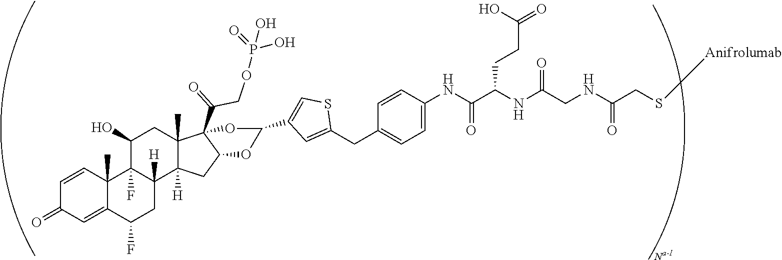 |
| II-C- 1047 | 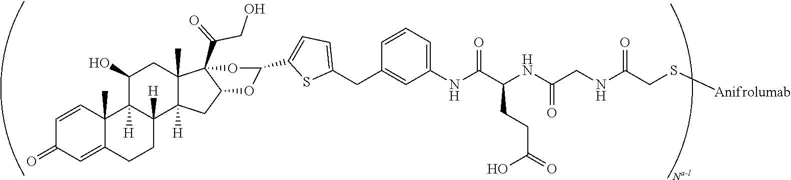 |
| II-C- 1048 | 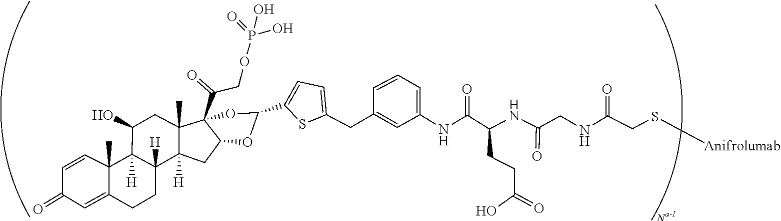 |
| II-C- 1052 | 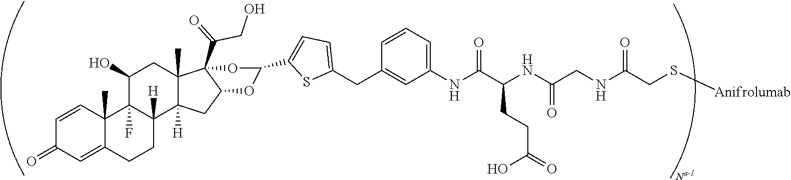 |
| II-C- 1053 | 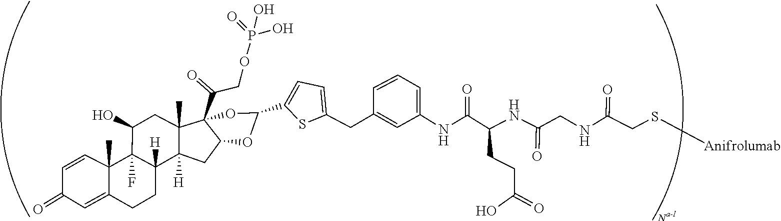 |
| II-C- 1057 | 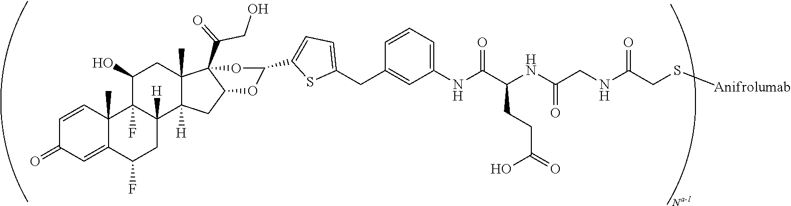 |
| II-C- 1058 | 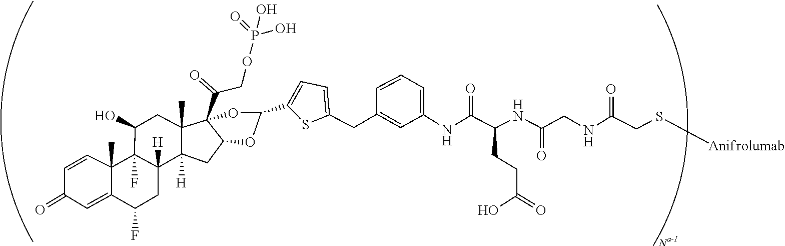 |
| II-C- 1062 | 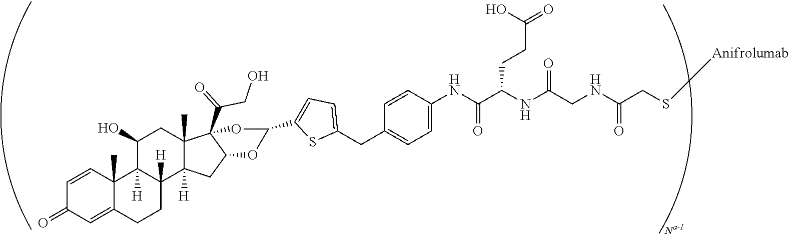 |
| II-C- 1063 | 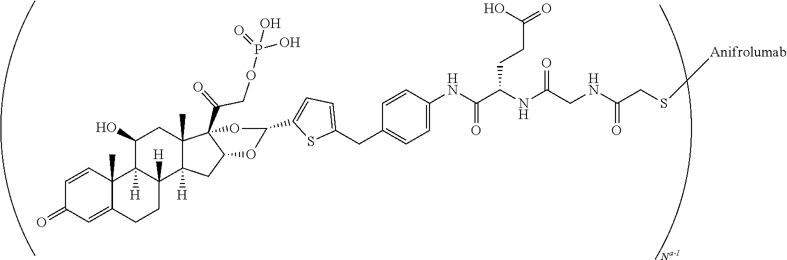 |
| II-C- 1067 | 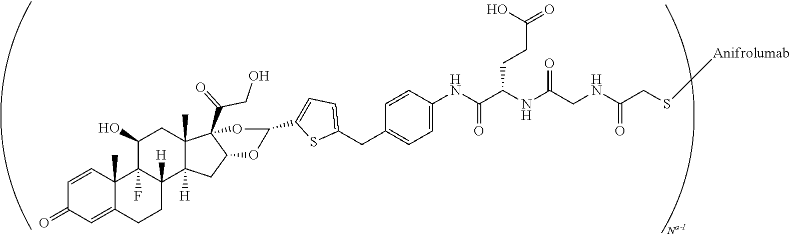 |
| II-C- 1068 | 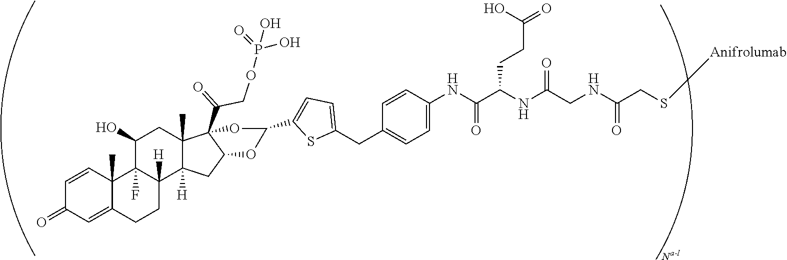 |
| II-C- 1072 | 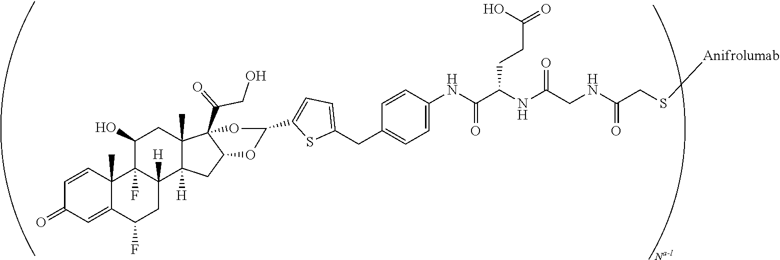 |
| II-C- 1073 | 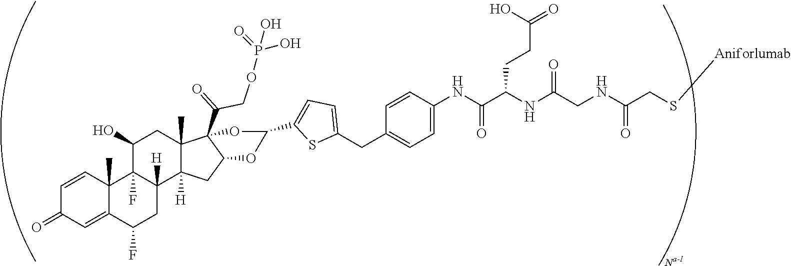 |
| II-C- 1085 | 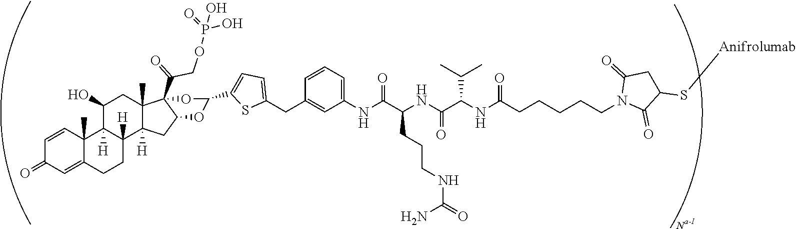 |
| II-C- 1086 | 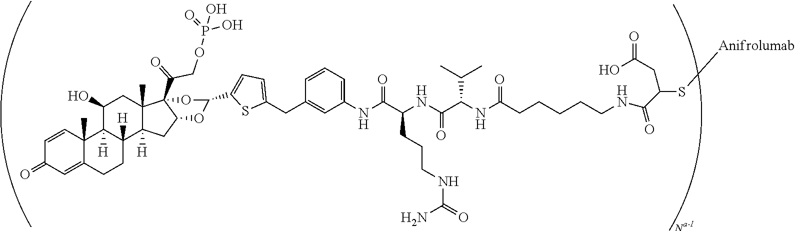 |
| II-C- 1087 | 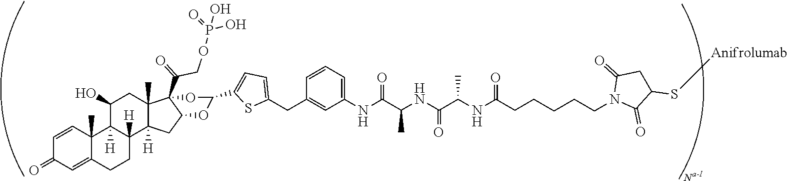 |
| II-C- 1088 | 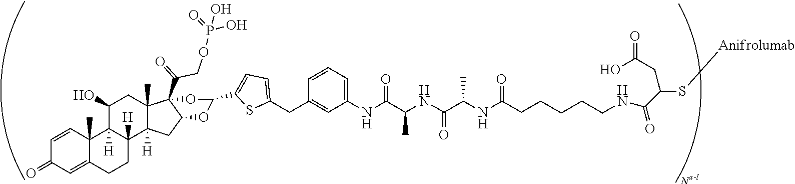 |
| II-C- 1089 | 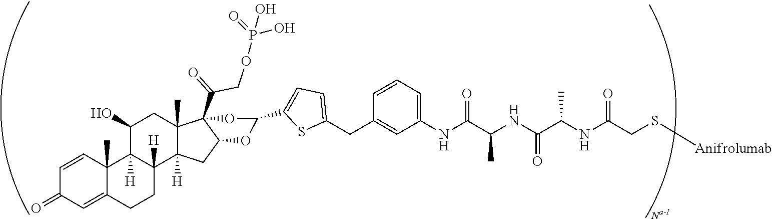 |
| II-C- 1135 | 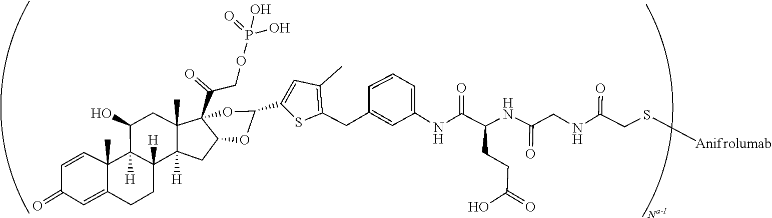 |
| II-C- 1138 | 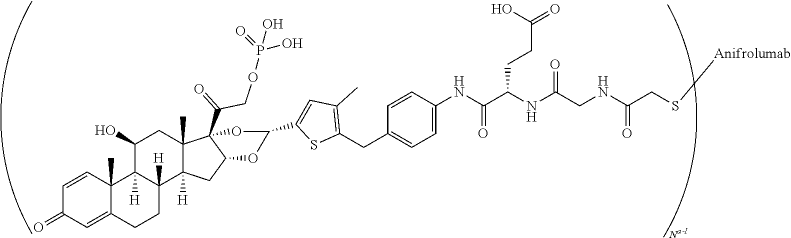 |
| II-C- 1141 | 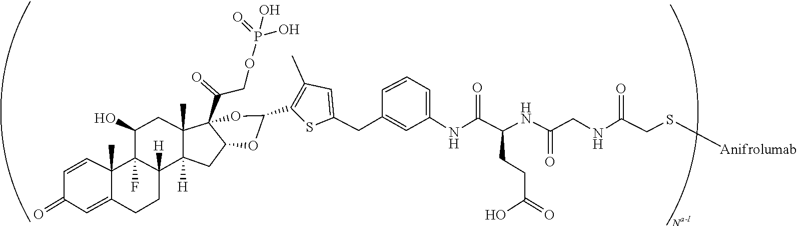 |
| II-C- 1144 | 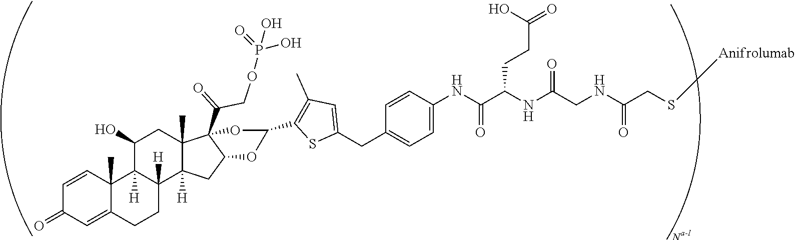 |
| II-C- 1147 | 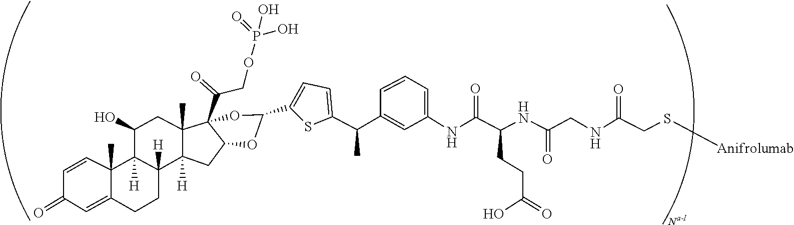 |
| II-C- 1150 | 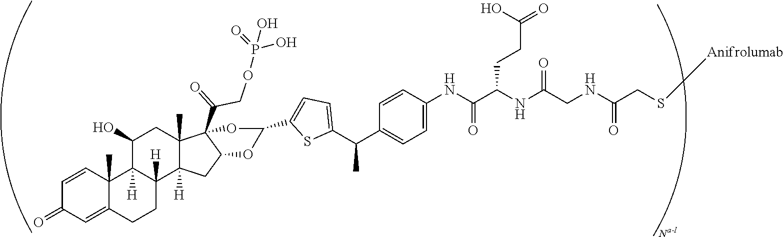 |
| II-C- 1153 | 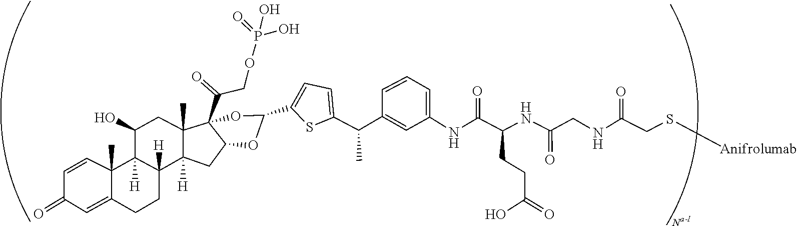 |
| II-C- 1156 | 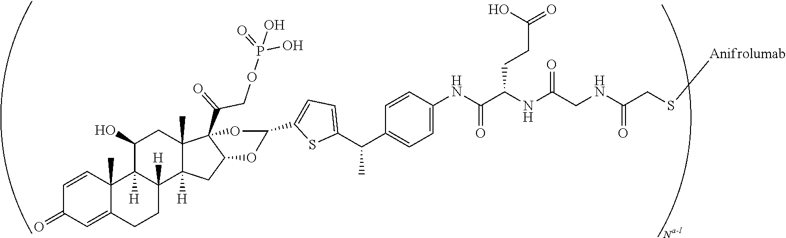 |
| II-C- 1159 | 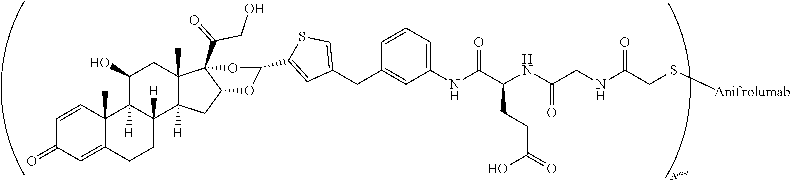 |
| II-C- 1160 | 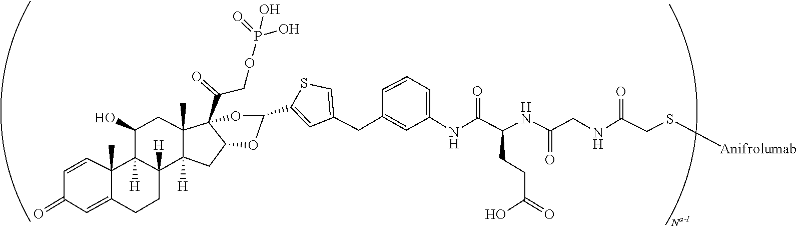 |
| II-C- 1174 | 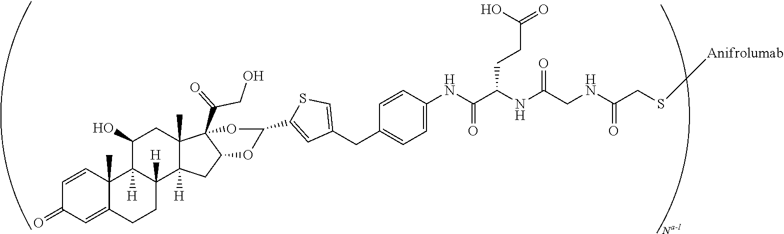 |
| II-C- 1175 | 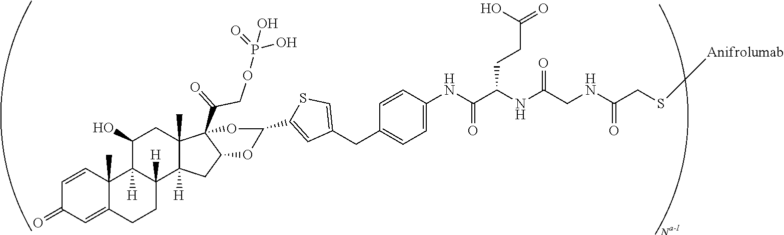 |
| II-C- 1189 | 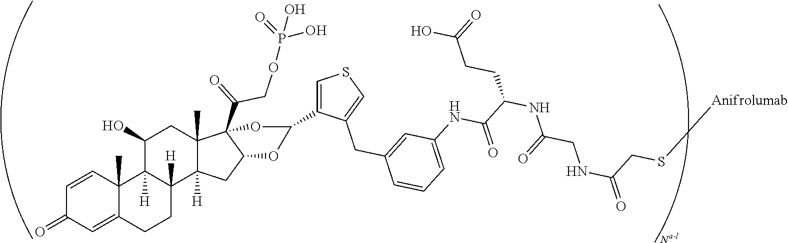 |
| II-C- 1190 | 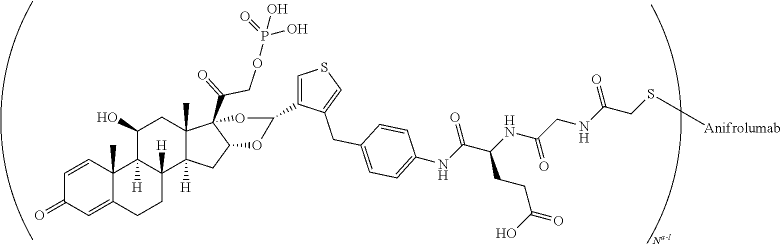 |
| II-C- 1191 | 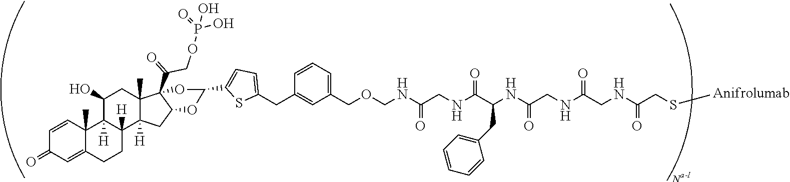 |
| II-C- 1192 | 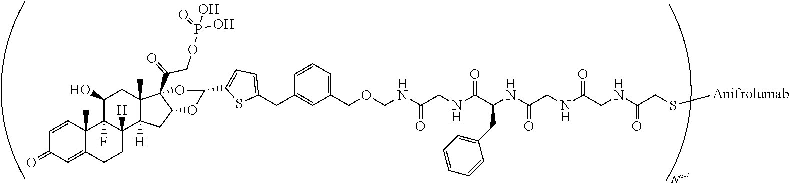 |
| II-C- 1193 | 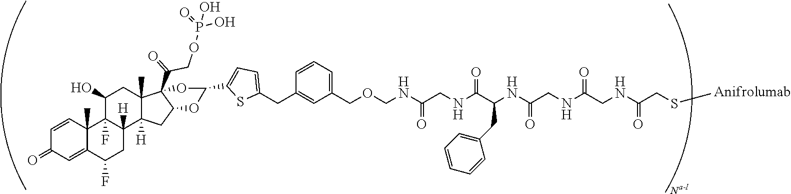 |
| II-C- 1194 | 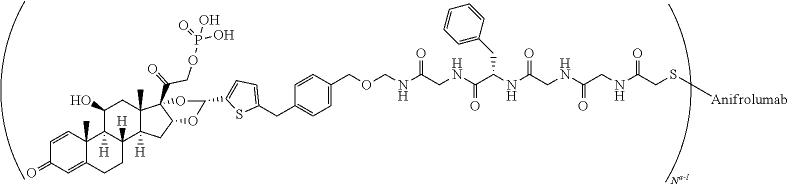 |
| II-C- 1195 | 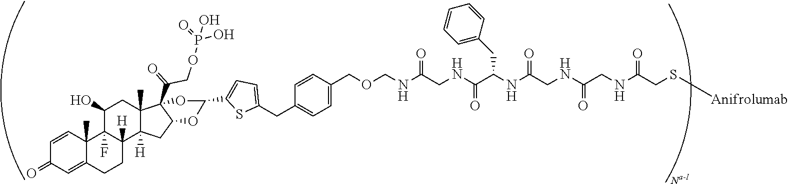 |
| II-C- 1196 | 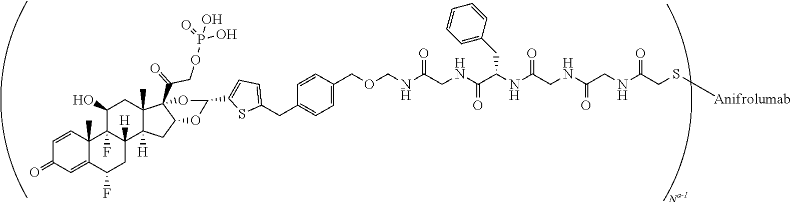 |
| II-C- 1197 | 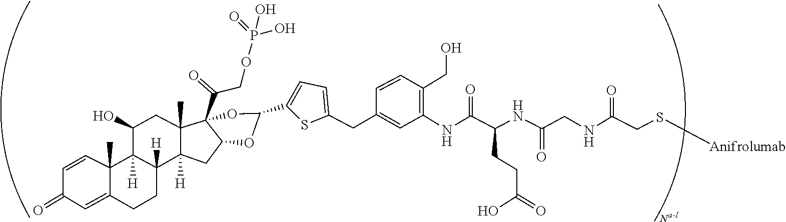 |
| II-C- 1200 | 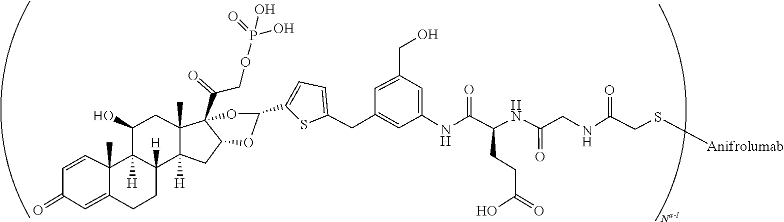 |
| II-C- 1203 | 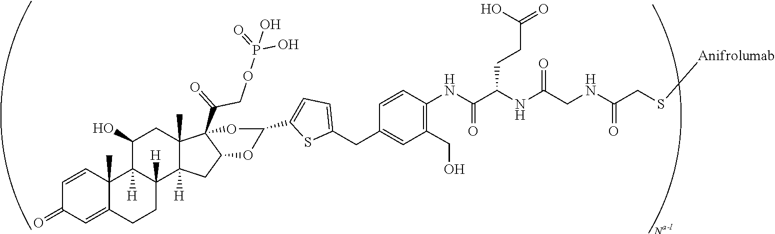 |
| II-C- 1206 | 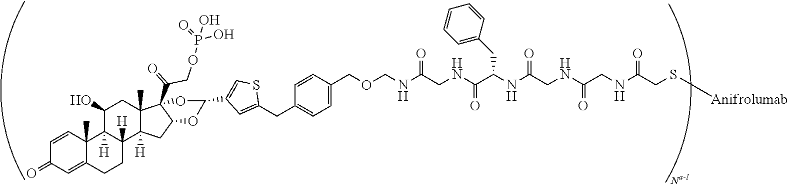 |
| II-C- 1207 | 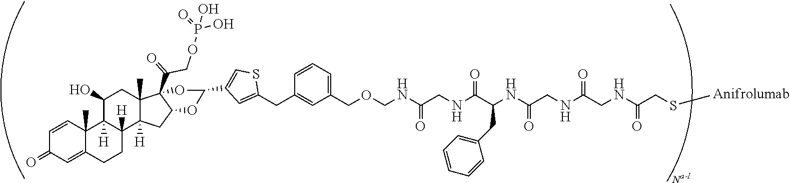 |
| II-C- 1208 | 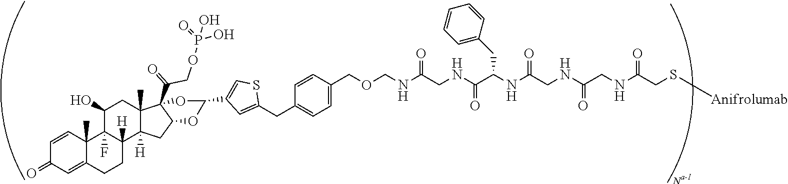 |
| II-C- 1209 | 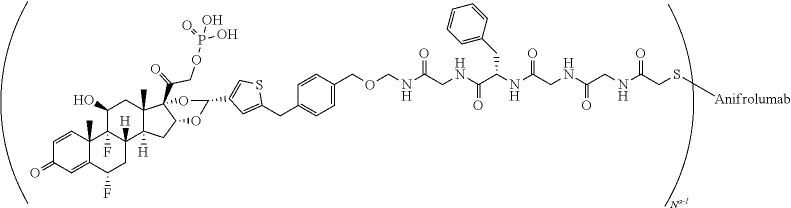 |
| II-C- 1210 | 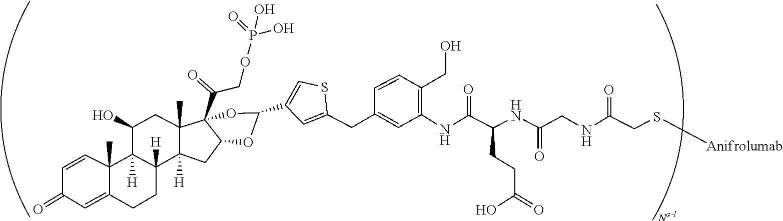 |
| II-C- 1213 | 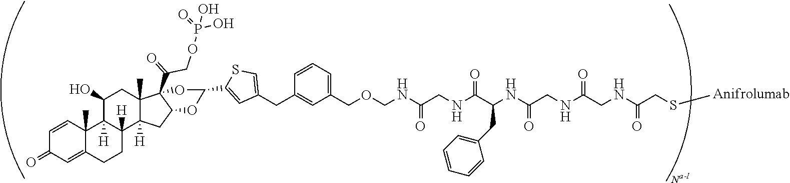 |
| II-C- 1214 | 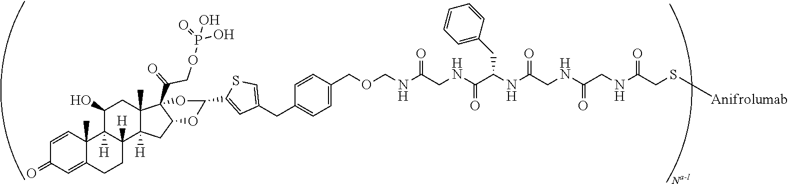 |
| II-C- 1215 | 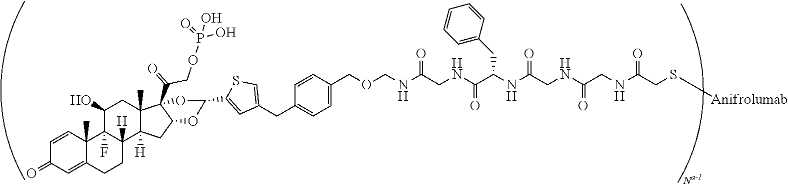 |
| II-C- 1216 | 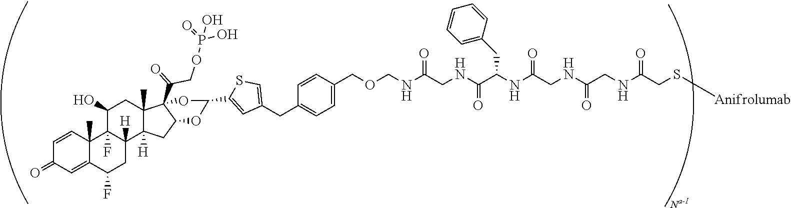 |
| II-C- 1217 | 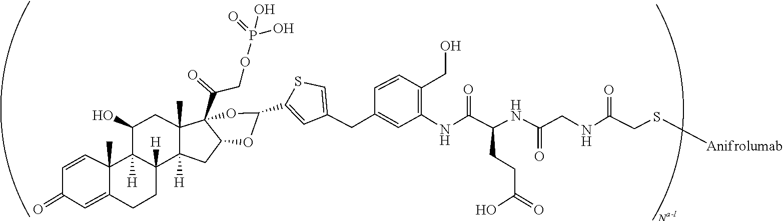 |
| II-C- 1220 | 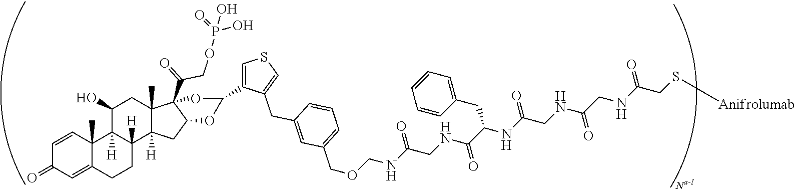 |
| II-C- 1221 | 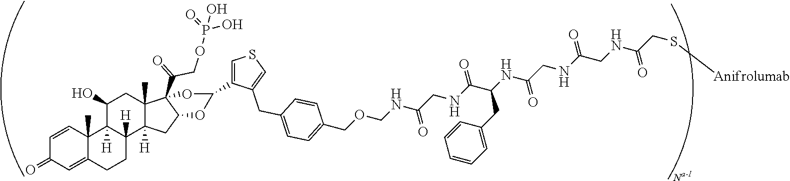 |

81. A compound or a tautomer, a mesomer, a racemate, an enantiomer or a diastereoisomer thereof, or a mixture thereof, or a pharmaceutically acceptable salt thereof, wherein the compound comprises formulas (II-a1-C-1), (II-a1-C-2), (II-a1-C-3), (II-a2-C-1), (II-a2-C-2), and (II-a2-C-3), 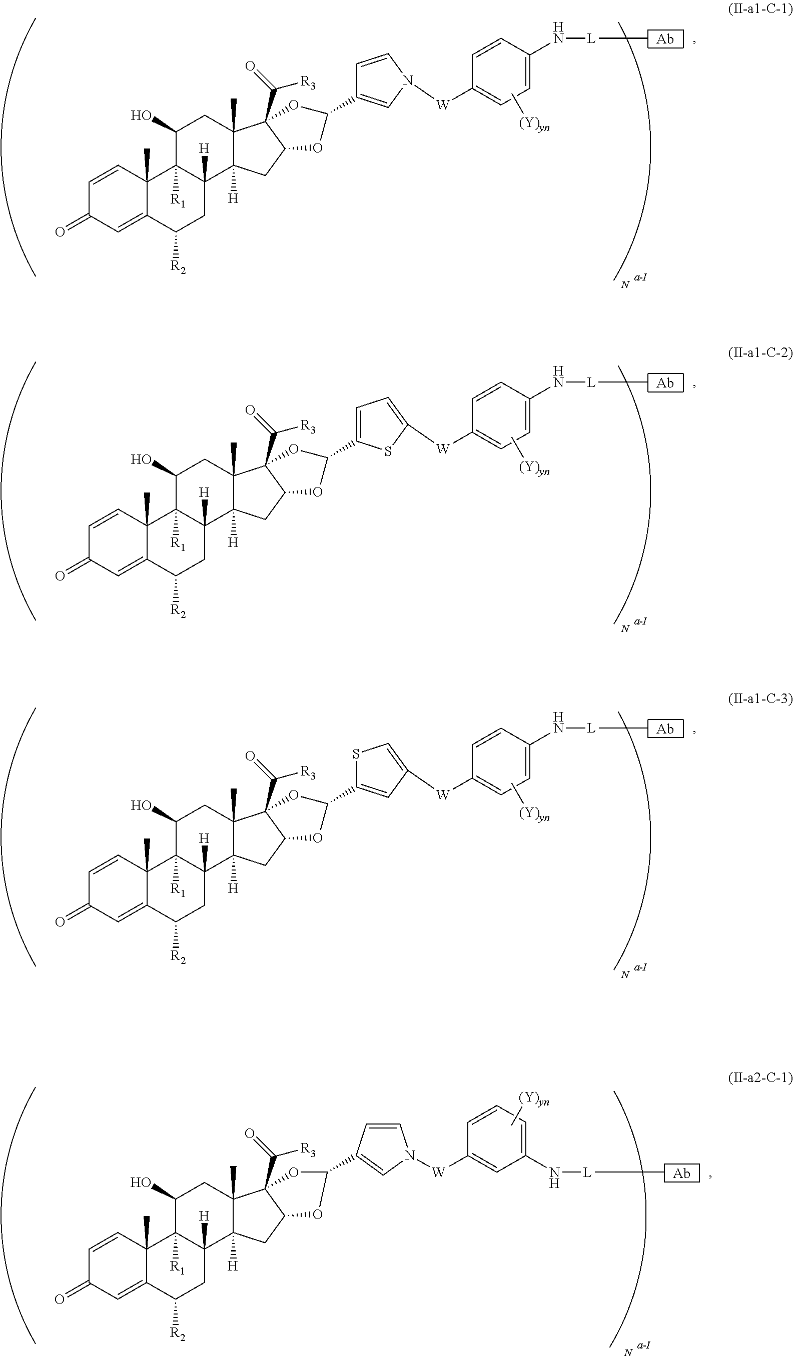 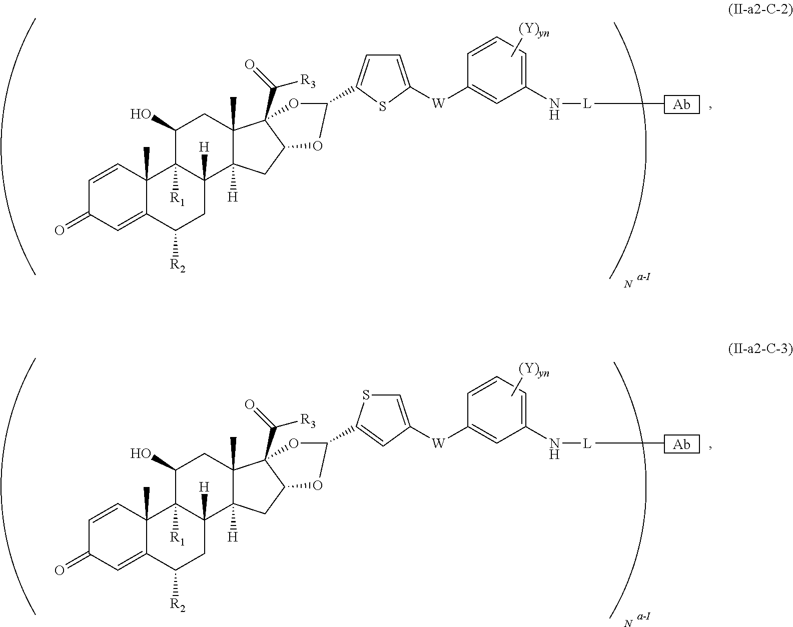
wherein,
R₁ and R₂ are each independently selected from: hydrogen, protium, deuterium, tritium, halogen, and C₁-C₆ alkyl;
R₃ is selected from: —CH₂Cl, —CH₂SH, —CH₂OH, 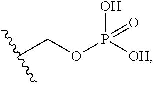 [SMILES: *COP(=O)(O)O]
—OCH₃, —OCH₂F, —OCH₂Cl, —OCH₂CN, —OCH₂CH₃, —SH, —SCH₂F, —SCH₂Cl, —SCH₂CF₃ and —SCH₂CN;
W is selected from 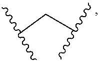 [SMILES: *C*]
—CH(CH₃)— and; 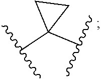 [SMILES: *C1(*)CC1]
N^{a}-1 is a number from about 0 to about 20;
in the formulas (II-a1-C-1) and (II-a2-C-1), Y is each independently selected from: hydrogen, halogen, hydroxy, sulfhydryl, amino, C₁-C₆ alkyl, and —OC₁-C₆ alkyl; in the formulas (II-a1-C-2), (II-a1-C-3), (II-a2-C-2) and (II-a2-C-2), Y is each independently selected from: hydrogen, halogen, hydroxy, sulfhydryl, amino, C₁-C₆ alkyl, —OC₁-C₆ alkyl and —C₁-C₆ alkylhydroxy;
yn is selected from 0, 1 or 2;
Ab is selected from an antibody or an antigen-binding fragment thereof;
L comprises -L³-L²-L¹-,
wherein L³ can be absent or selected from the group consisting of: glutamic acid-glycine (EG), alanine-alanine (AA), alanine-glutamic acid (AE), glycine-glutamic acid-glycine (GEG), glycine-alanine-glutamic acid (GAE), glycine-phenylalanine-glycine-glycine (GFGG), and citrulline-valine (CV),
L² can be absent or selected from the group consisting of: 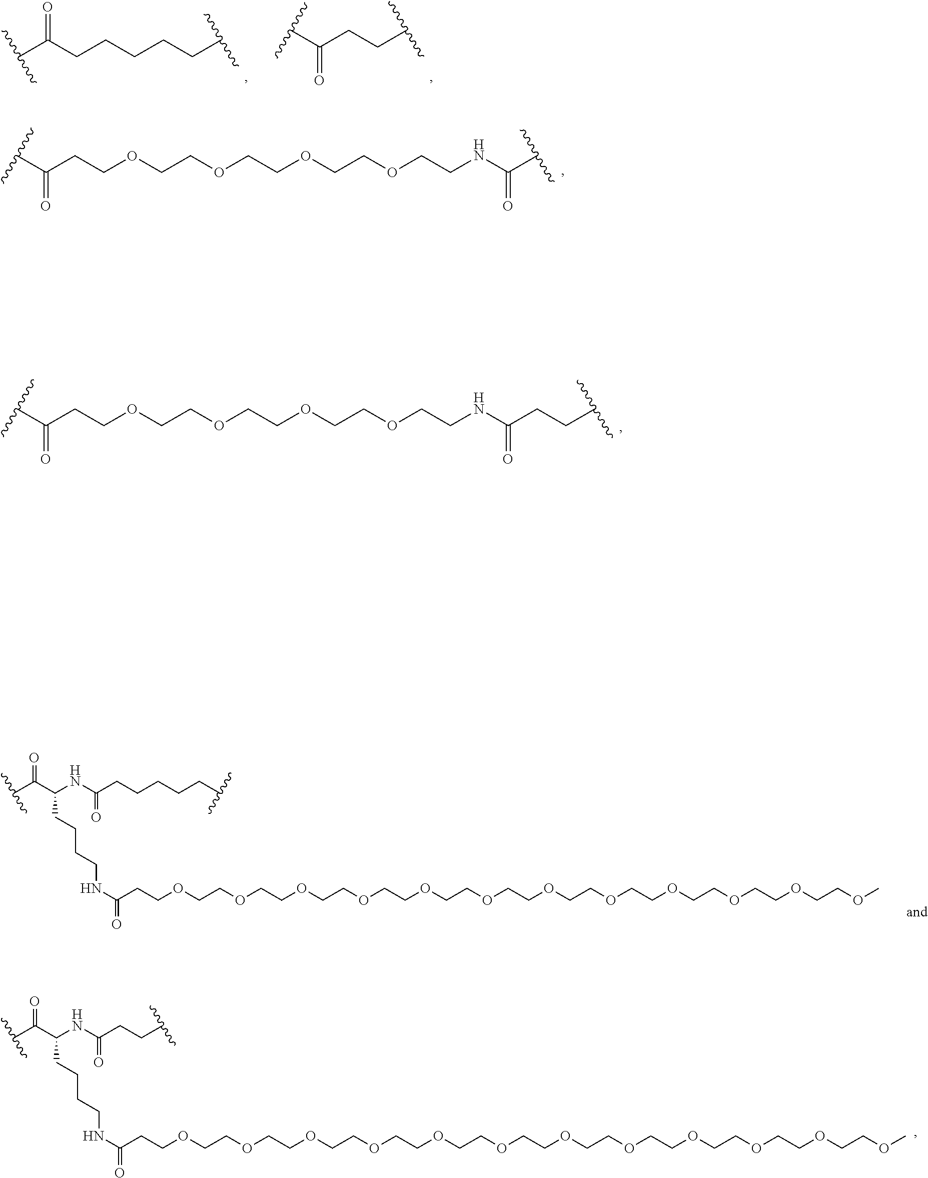 [SMILES: *CCCCCC(*)=O ; *CCC(*)=O ; *C(=O)CCOCCOCCOCCOCCNC(*)=O ; *CCC(=O)NCCOCCOCCOCCOCCC(*)=O ; *CCCCCC(=O)N[C@H](CCCCNC(=O)CCOCCOCCOCCOCCOCCOCCOCCOCCOCCOCCOCCOC)C(*)=O ; *CCC(=O)N[C@H](CCCCNC(=O)CCOCCOCCOCCOCCOCCOCCOCCOCCOCCOCCOCCOC)C(*)=O]
and
L¹ can be selected from the group consisting of: 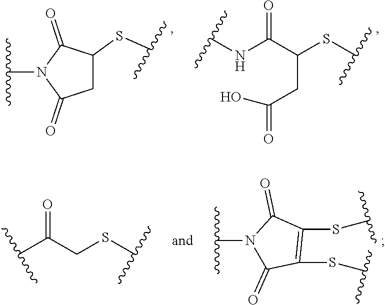
preferably L is

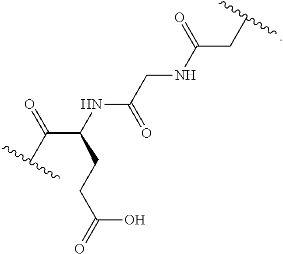 [SMILES: *CC(=O)NCC(=O)N[C@@H](CCC(=O)O)C(*)=O]

82. The compound or the tautomer, the mesomer, the racemate, the enantiomer or the diastereoisomer thereof, or the mixture thereof, or the pharmaceutically acceptable salt thereof according to claim 81, wherein the compound is selected from: 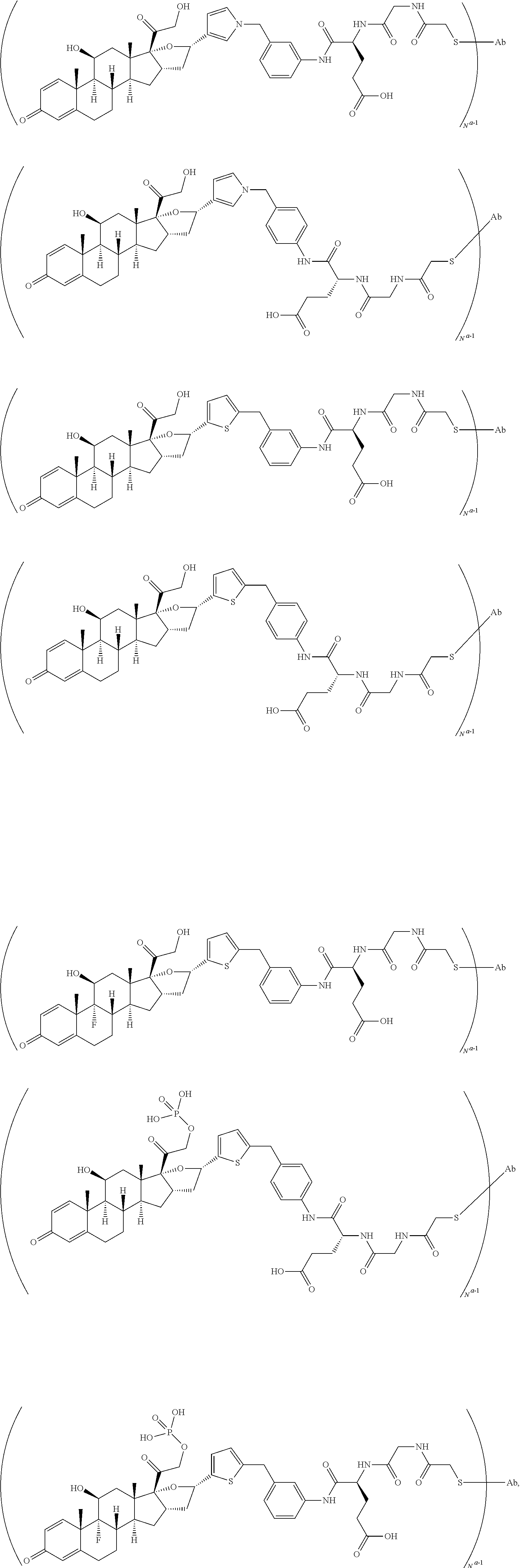
wherein N^{a-1} is a number from about 0 to about 20, and Ab is selected from an antibody or an antigen-binding fragment thereof.

83. The compound or the tautomer, the mesomer, the racemate, the enantiomer or the diastereoisomer thereof, or the mixture thereof, or the pharmaceutically acceptable salt thereof according to claim 82, wherein the compound is selected from: 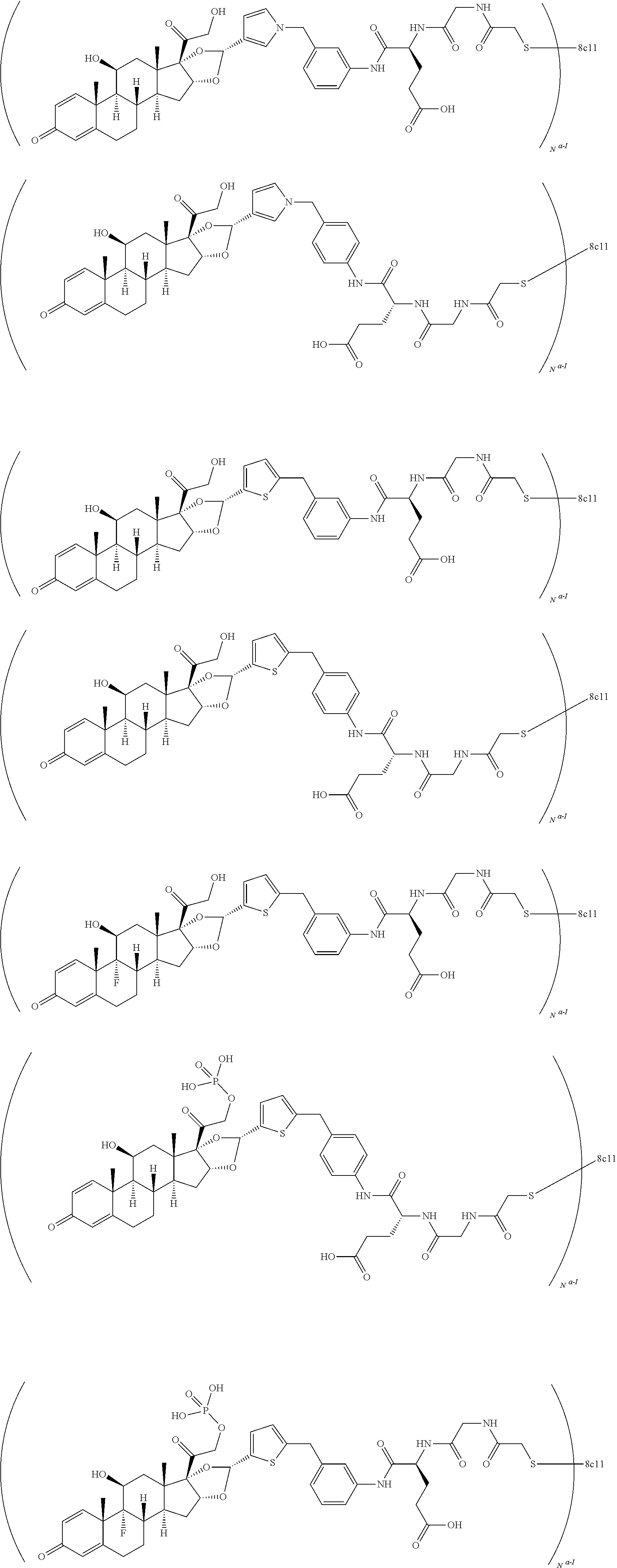 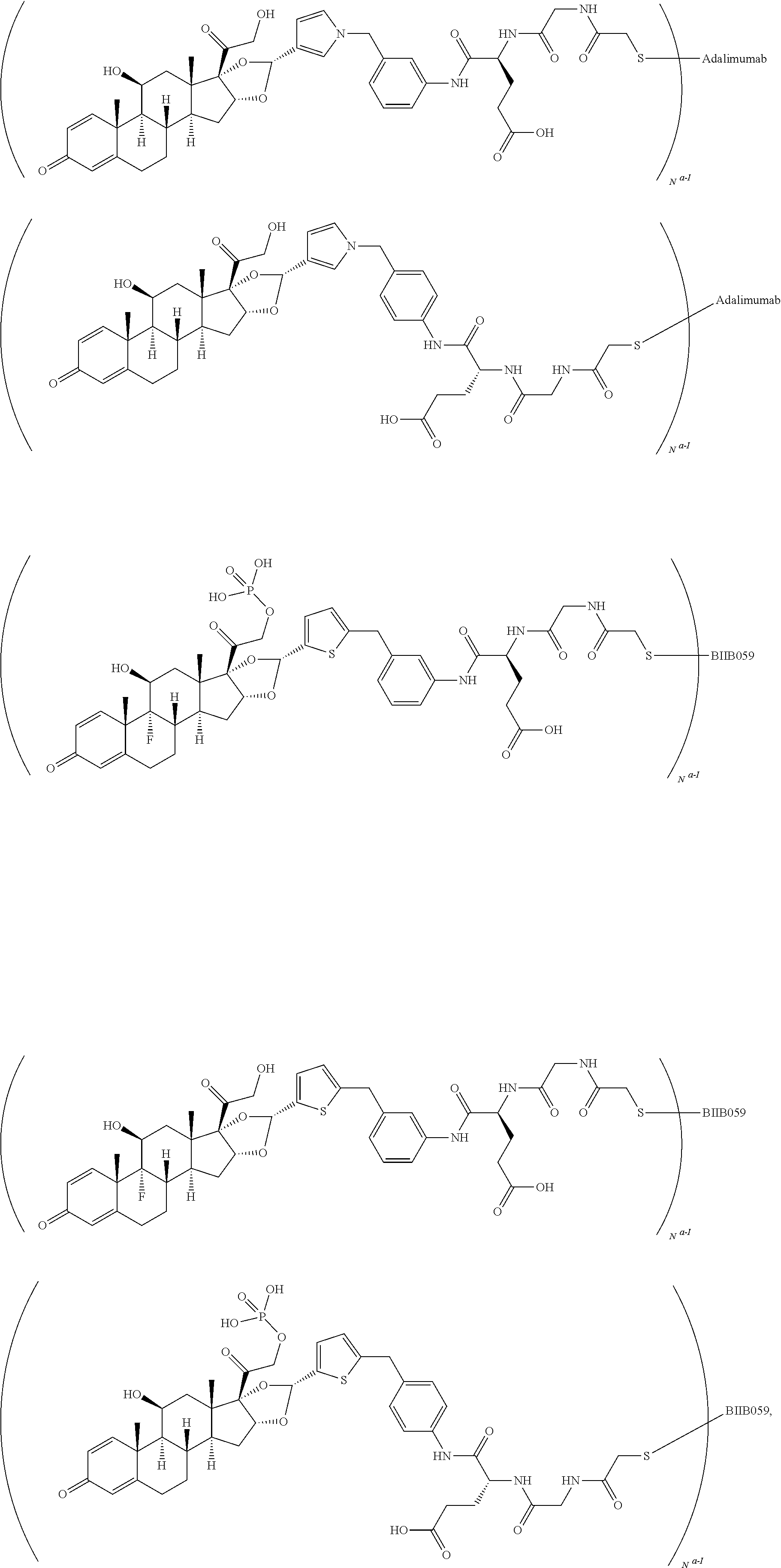
wherein N^{a-1} is a number from about 0 to about 20.

84. A pharmaceutical composition, comprising the compound or the tautomer, the mesomer, the racemate, the enantiomer or the diastereoisomer thereof, or the mixture thereof, or the pharmaceutically acceptable salt thereof according to any one of claims 1-26 and 28-83 and/or the conjugate according to any one of claims 27-28, and optionally a pharmaceutically acceptable carrier.

85. A method for influencing immune system functions, comprising administering to a subject the compound or the tautomer, the mesomer, the racemate, the enantiomer or the diastereoisomer thereof, or the mixture thereof, or the pharmaceutically acceptable salt thereof according to any one of claims 1-26 and 29-83, the conjugate according to any one of claims 27-28, and/or the pharmaceutical composition according to claim 84.

86. Use of the compound or the tautomer, the mesomer, the racemate, the enantiomer or the diastereoisomer thereof, or the mixture thereof, or the pharmaceutically acceptable salt thereof according to any one of claims 1-26 and 29-83, the conjugate according to any one of claims 27-28, and/or the pharmaceutical composition according to claim 84 in preparing a medicament for preventing and/or treating a disease and/or condition, wherein the disease and/or condition comprises diseases and/or conditions associated with glucocorticoid receptor signaling.

87. The use according to claim 86, wherein the disease and/or condition are selected from the group consisting of: proliferative diseases and/or conditions, metabolic diseases and/or conditions, inflammatory diseases and/or conditions, neurodegenerative diseases and/or conditions, systemic autoimmune diseases and/or conditions, blood system-related diseases and/or conditions, neuromuscular system-related diseases and/or conditions, digestive system-related diseases and/or conditions, urological system-related diseases and/or conditions, endocrine gland system-related diseases and/or conditions, cutaneous muscular system-related diseases and/or conditions, respiratory system-related diseases and/or conditions, rheumatoid arthritis, systemic lupus erythematosus, scleroderma, Sjogren's syndrome, ankylosing spondylitis, Wegener granulomatosis, systemic sclerosis, autoimmune hemolytic anemia, pernicious anemia, idiopathic thrombocytopenic purpura, idiopathic thrombocytopenia, vasculitis, multiple sclerosis, myasthenia gravis, Guillain-Barre syndrome, ulcerative colitis, Crohn's disease, autoimmune liver disease, atrophic gastritis, IgA nephropathy, primary nephrotic syndrome, autoimmune glomerulonephritis, Goodpasture's syndrome, lupus nephritis, type I diabetes, Grave's disease, Hashimoto thyroiditis, primary adrenocortical atrophy, chronic thyroiditis, psoriasis, pemphigus vulgaris, cutaneous lupus erythematosis, dermatomyositis, polymyalgia rheumatica, and asthma.


SEQUENCE LISTING (42 sequences)

SEQ ID NO: 1 - protein, 11 aa, synthetic construct
  REGION 1..11: note = Humira LCDR1
RASQGIRNYLA

SEQ ID NO: 2 - protein, 7 aa, synthetic construct
  REGION 1..7: note = Humira LCDR2
AASTLQS

SEQ ID NO: 3 - protein, 9 aa, synthetic construct
  REGION 1..9: note = Humira LCDR3
QRYNRAPYT

SEQ ID NO: 4 - protein, 5 aa, synthetic construct
  REGION 1..5: note = Humira HCDR1
DYAMH

SEQ ID NO: 5 - protein, 17 aa, synthetic construct
  REGION 1..17: note = Humira HCDR2
AITWNSGHIDYADSVEG

SEQ ID NO: 6 - protein, 14 aa, synthetic construct
  REGION 1..14: note = Humira HCDR3
AKVSYLSTASSLDY

SEQ ID NO: 7 - protein, 107 aa, synthetic construct [sequence omitted]

SEQ ID NO: 8 - protein, 121 aa, synthetic construct [sequence omitted]

SEQ ID NO: 9 - protein, 214 aa, synthetic construct
  REGION 1..214: note = Humira light chain
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 451 aa, synthetic construct
  REGION 1..451: note = Humira heavy chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 16 aa, synthetic construct
  REGION 1..16: note = Iscalimab LCDR1
RSSQSLLYSNGYNYLD

SEQ ID NO: 12 - protein, 7 aa, synthetic construct
  REGION 1..7: note = Iscalimab LCDR2
LGSNRAS

SEQ ID NO: 13 - protein, 9 aa, synthetic construct
  REGION 1..9: note = Iscalimab LCDR3
MQARQTPFT

SEQ ID NO: 14 - protein, 5 aa, synthetic construct
  REGION 1..5: note = Iscalimab HCDR1
SYGMH

SEQ ID NO: 15 - protein, 17 aa, synthetic construct
  REGION 1..17: note = Iscalimab HCDR2
VISYEESNRYHADSVKG

SEQ ID NO: 16 - protein, 11 aa, synthetic construct
  REGION 1..11: note = Iscalimab HCDR3
DGGIAAPGPDY

SEQ ID NO: 17 - protein, 120 aa, synthetic construct
  REGION 1..120: note = Iscalimab VH
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 18 - protein, 112 aa, synthetic construct
  REGION 1..112: note = Iscalimab VL
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 19 - protein, 219 aa, synthetic construct
  REGION 1..219: note = Iscalimab light chain
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 450 aa, synthetic construct
  REGION 1..450: note = Iscalimab heavy chain
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 12 aa, synthetic construct
  REGION 1..12: note = Anifrolumab LCDR1
RASQSVSSSFFA

SEQ ID NO: 22 - protein, 7 aa, synthetic construct
  REGION 1..7: note = Anifrolumab LCDR2
GASSRAT

SEQ ID NO: 23 - protein, 9 aa, synthetic construct
  REGION 1..9: note = Anifrolumab LCDR3
QQYDSSAIT

SEQ ID NO: 24 - protein, 5 aa, synthetic construct
  REGION 1..5: note = Anifrolumab HCDR1
NYWIA

SEQ ID NO: 25 - protein, 17 aa, synthetic construct
  REGION 1..17: note = Anifrolumab HCDR2
IIYPGDSDIRYSPSFQG

SEQ ID NO: 26 - protein, 8 aa, synthetic construct
  REGION 1..8: note = Anifrolumab HCDR3
HDIEGFDY

SEQ ID NO: 27 - protein, 117 aa, synthetic construct [sequence omitted]

SEQ ID NO: 28 - protein, 108 aa, synthetic construct [sequence omitted]

SEQ ID NO: 29 - protein, 215 aa, synthetic construct
  REGION 1..215: note = Anifrolumab light chain
EIVLTQSPGTLSLSPGERATLSCRASQSVSSSFFAWYQQKPGQAPRLLIYGASSRATGIPDRLSGSGSGTDFTLTITRLEPEDFAVYYCQQYDSSAITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 447 aa, synthetic construct
  REGION 1..447: note = Anifrolumab heavy chain
EVQLVQSGAEVKKPGESLKISCKGSGYIFTNYWIAWVRQMPGKGLESMGIIYPGDSDIRYSPSFQGQVTISADKSITTAYLQWSSLKASDTAMYYCARHDIEGFDYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 213 aa, synthetic construct
  REGION 1..213: note = anti-mice TNF mIgG2a 8C11light chain
QIVLSQSPAILSASPGEKVTMTCRASSSVSYMHWFQQKPGSSPKPWIYATSNLASGVPARFSGSGSGTSYSLTISRVEAEDAATYYCQQWSSSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 451 aa, synthetic construct
  REGION 1..451: note = anti-mice TNF mIgG2a 8C11 heavy chain
EFQLQQSGPELVKPGASVRISCKASGYSFTDYNMNWVKQSNGKSLEWVGVINPNYGSSTYNQKFKGKATLTVDQSSSTAYMQLNSLTSEDSAVYYCARKWGQLGRGFFDVWGTGTTVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 12 aa, synthetic construct
  REGION 1..12: note = BIIB059 HCDR3
DIYYNYGAWFAY

SEQ ID NO: 34 - protein, 7 aa, synthetic construct
  REGION 1..7: note = BIIB059 HCDR2
SPGDSFG

SEQ ID NO: 35 - protein, 7 aa, synthetic construct
  REGION 1..7: note = BIIB059 HCDR1
GFTFSTY

SEQ ID NO: 36 - protein, 122 aa, synthetic construct
  REGION 1..122: note = BIIB059 VH
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSS

SEQ ID NO: 37 - protein, 451 aa, synthetic construct
  REGION 1..451: note = BIIB059 heavy chain
DVQLVESGGGLVKPGGSLRLSCAASGFTFSTYTMSWVRQAPGKGLEWVATISPGDSFGYYYPDSVQGRFTISRDNAKNSLYLQMNSLRAEDTAVYYCTRDIYYNYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 9 aa, synthetic construct
  REGION 1..9: note = BIIB059 LCDR3
QQANEDPRT

SEQ ID NO: 39 - protein, 7 aa, synthetic construct
  REGION 1..7: note = BIIB059 LCDR2
AASTLES

SEQ ID NO: 40 - protein, 15 aa, synthetic construct
  REGION 1..15: note = BIIB059 LCDR1
KASQSVDYDGDSYMN

SEQ ID NO: 41 - protein, 111 aa, synthetic construct
  REGION 1..111: note = BIIB059 VL
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIK

SEQ ID NO: 42 - protein, 218 aa, synthetic construct
  REGION 1..218: note = BIIB059 light chain
DIQLTQSPSSLSASVGDRVTITCKASQSVDYDGDSYMNWYQQKPGKAPKLLIYAASTLESGVPSRFSGSGSGTDFTLTISSLQPEDFATYYCQQANEDPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC